(19) United States
(12) Patent Application Publication
(10) Pub. No.: US 2024/0081148 A1
(43) Pub. Date: Mar. 7, 2024

(54) ORGANIC LIGHT EMITTING DEVICE

(71) Applicant: LG CHEM, LTD., Seoul (KR)

(72) Inventors: Minjun KIM, Daejeon (KR); Dong Hoon LEE, Daejeon (KR); Sang Duk SUH, Daejeon (KR); Young Seok KIM, Daejeon (KR); Donghee KIM, Daejeon (KR); Joongsuk OH, Daejeon (KR); Da Jung LEE, Daejeon (KR)

(21) Appl. No.: 18/038,788

(22) PCT Filed: Apr. 27, 2022
(86) PCT No.: PCT/KR2022/006053

§ 371 Date: May 26, 2023

(30) Foreign Application Priority Data
Apr. 27, 2021 (KR) 10-2021-0054555
Apr. 27, 2022 (KR) 10-2022-0052253

(51) Int. Cl. H10K 85/60; C07D 307/77; C07D 307/91; C07D 405/04; C07D 405/10; C07D 405/12; C07D 405/14; C07D 409/12; C07D 409/14; C07F 7/08; C09K 11/06; H10K 85/40

(52) U.S. Cl. CPC H10K 85/6574; C07D 307/77; C07D 307/91; C07D 405/04; C07D 405/10; C07D 405/12; C07D 405/14; C07D 409/12; C07D 409/14; C07F 7/0812; C09K 11/06; H10K 85/40; H10K 85/615; H10K 85/626; H10K 85/633; H10K 85/636; H10K 85/654; H10K 85/6576; H10K 50/11

(57) ABSTRACT

Provided is an organic light-emitting device having improved driving voltage, efficiency and lifespan, the device comprising an anode, a cathode, and a light emitting layer including a light emitting layer that includes a compound of Chemical Formula 1 and a compound of Chemical Formula 2 between the anode and the cathode:

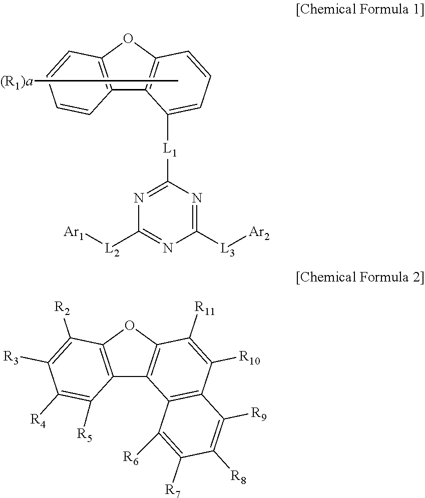

where Ar₁ and Ar₂ are each independently a substituted or unsubstituted C₆₋₆₀ aryl or C₂₋₆₀ heteroaryl containing at least one of N, O and S; R₁ is each independently hydrogen or deuterium; R₂ to R₆ and R₉ to R₁₁ are each independently hydrogen or deuterium; one of R₇ and R₈ is

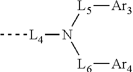

and the other is hydrogen or deuterium; Ar₃ and Ar₄ are each independently a substituted or unsubstituted C₆₋₆₀ aryl or C₂₋₆₀ heteroaryl containing at least one of N, O and S; and the other substituents are as defined in the specification.

### CROSS-REFERENCE TO RELATED APPLICATION(S)

This application is a National Stage Application of International Application No. PCT/KR2022/006053 filed on Apr. 27, 2022, which claims priority to and the benefit of Korean Patent Application No. 10-2021-0054555 filed on Apr. 27, 2021 and Korean Patent Application No. 10-2022-0052253 filed on Apr. 27, 2022 in the Korean Intellectual Property Office, the contents of which are incorporated herein by reference in their entirety.

### TECHNICAL FIELD

The present disclosure relates to an organic light emitting device having improved driving voltage, efficiency and lifetime.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuing need for the development of a new material for an organic material used in the organic light emitting device as described above.

### PRIOR ART LITERATURE

### Patent Literature

(Patent Literature 1) Korean Unexamined Patent Publication No. 10-2000-0051826

### DETAILED DESCRIPTION OF THE INVENTION

### Technical Problem

It is an object of the present disclosure to provide an organic light emitting device having improved driving voltage, efficiency and lifetime.

### Technical Solution

Provided herein is the following organic light emitting device:

An organic light emitting device including: an anode, a cathode, and a light emitting layer interposed between the anode and the cathode, wherein the light emitting layer includes a compound of

Chemical Formula 1 and a compound of Chemical Formula 2:

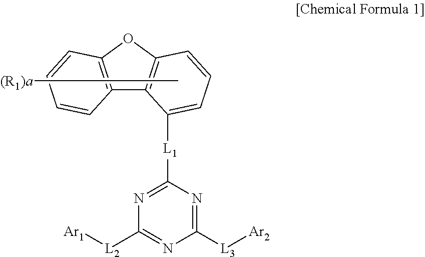

wherein in the Chemical Formula 1:
Ar₁ and Ar₂ are each independently a substituted or unsubstituted C₆₋₆₀ aryl, or a substituted or unsubstituted C₂₋₆₀ heteroaryl containing at least one heteroatom selected from the group consisting of N, O and S;
L₁ to L₃ are each independently a single bond or a substituted or unsubstituted C₆₋₆₀ arylene;
R₁ is each independently hydrogen or deuterium; and
a is an integer of 0 to 7;

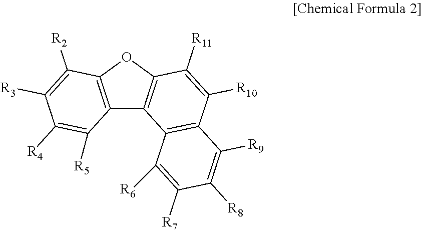

wherein in the Chemical Formula 2:
R₂ to R₆ and R₉ to R₁₁ are each independently hydrogen or deuterium;
any one of R₇ and R₈ is

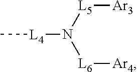

and the other is hydrogen or deuterium;
Ar₃ and Ar₄ are each independently a substituted or unsubstituted C₆₋₆₀ aryl, or a substituted or unsubstituted C₂₋₆₀ heteroaryl containing at least one heteroatom selected from the group consisting of N, O and S;
L₄ is a substituted or unsubstituted phenylene, a substituted or unsubstituted biphenyldiyl, or a substituted or unsubstituted naphthalenediyl; and
L₅ and L₆ are each independently a single bond, a substituted or unsubstituted C₆₋₆₀ arylene, or a substituted or unsubstituted C₂₋₆₀ heteroarylene containing at least one heteroatom selected from the group consisting of N, O and S.

### Advantageous Effects

The above-mentioned organic light emitting device includes the compound of Formula 1 and the compound of Chemical Formula 2 in the light emitting layer, and thus can improve the efficiency, achieve low driving voltage and/or improve lifetime characteristics in the organic light emitting device.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron injection and transport layer 9, and a cathode 4.

### DETAILED DESCRIPTION OF THE EMBODIMENTS

Hereinafter, embodiments of the present disclosure will be described in more detail to facilitate understanding of the invention.

As used herein, the notation

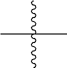

or [symbol] means a bond linked to another substituent group.

As used herein, the term “substituted or unsubstituted” means being unsubstituted or substituted with one or more substituents selected from the group consisting of deuterium, a halogen group, a nitrile group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heterocyclic group containing at least one of N, 0 and S atoms, or being unsubstituted or substituted with a substituent to which two or more substituents of the above-exemplified substituents are connected. For example, “a substituent in which two or more substituents are connected” can be a biphenyl group. Namely, a biphenyl group can be an aryl group, or it can be interpreted as a substituent in which two phenyl groups are connected.

In the present disclosure, the carbon number of a carbonyl group is not particularly limited, but is preferably 1 to 40. Specifically, the carbonyl group can be a compound having the following structural formulas, but is not limited thereto:

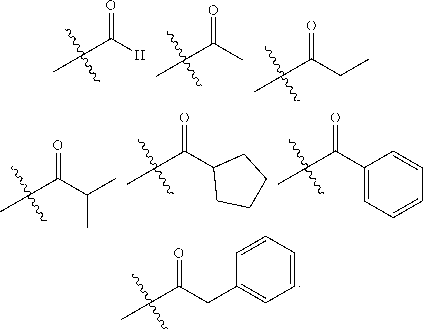

In the present disclosure, an ester group can have a structure in which oxygen of the ester group can be substituted by a straight-chain, branched-chain, or cyclic alkyl group having 1 to 25 carbon atoms, or an aryl group having 6 to 25 carbon atoms. Specifically, the ester group can be a compound having the following structural formulas, but is not limited thereto:

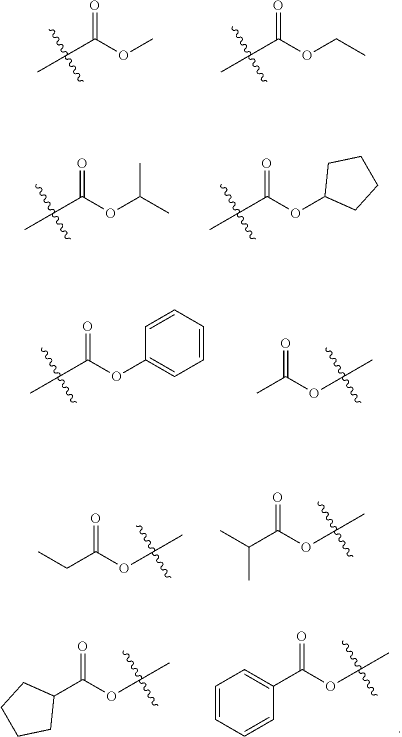

In the present disclosure, the carbon number of an imide group is not particularly limited, but is preferably 1 to 25. Specifically, the imide group can be a compound having the following structural formulas, but is not limited thereto:

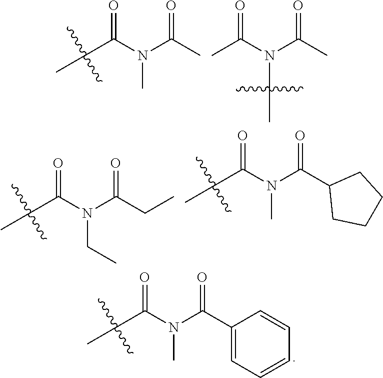

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group can be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The aryl group can be a phenyl group, a biphenyl group, a terphenyl group or the like as the monocyclic aryl group, but is not limited thereto. The polycyclic aryl group includes a naphthyl group, an anthracenyl group, a phenanthrenyl group, a pyrenyl group, a perylenyl group, a chrysenyl group, or the like, but is not limited thereto.

In the present disclosure, the fluorenyl group can be substituted, and two substituents can be linked with each other to form a spiro structure. In the case where the fluorenyl group is substituted,

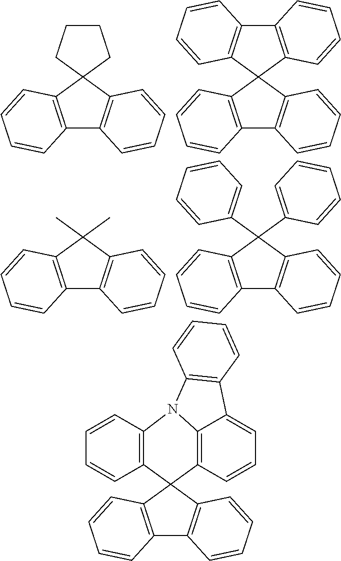

and the like can be formed. However, the structure is not limited thereto.

In the present disclosure, a heterocyclic group is a heterocyclic group containing one or more of O, N, Si and S as a heteroatom, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can be applied to the aforementioned description of the heterocyclic group. In the present disclosure, the alkenyl group in the aralkenyl group is the same as the aforementioned examples of the alkenyl group. In the present disclosure, the aforementioned description of the aryl group can be applied except that the arylene is a divalent group. In the present disclosure, the aforementioned description of the heterocyclic group can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heterocyclic group can be applied, except that the heterocycle is not a monovalent group but formed by combining two substituent groups.

In the present disclosure, the compound represented by ‘[structural formula]_{Dn}’ means a compound in which n hydrogens are substituted with deuterium among compounds having the corresponding ‘structural formula’.

Hereinafter, the present disclosure will be described in detail for each configuration.

Anode and Cathode

An anode and a cathode used in the present disclosure mean electrodes used in an organic light emitting device.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SNO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/AI or LiO₂/Al, and the like, but are not limited thereto.

Hole Injection Layer

The organic light emitting device according to the present

disclosure can further include a hole injection layer on the anode, if necessary.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and further is excellent in the ability to form a thin film. Further, it is preferable that a HOMO (highest occupied molecular orbital) of the hole injection material is between the work function of the anode material and a HOMO of a peripheral organic material layer.

Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive compound, and the like, but are not limited thereto.

Hole Transport Layer

The organic light emitting device according to the present disclosure can include a hole transport layer on the anode (or on the hole injection layer if the hole injection layer exists), if necessary.

The hole transport layer is a layer that can receive the holes from the anode or the hole injection layer and transport the holes to the light emitting layer, and the hole transport material is suitably a material having large mobility to the holes, which can receive holes from the anode or the hole injection layer and transfer the holes to the light emitting layer.

Specific examples thereof include an arylamine-based organic material, a conductive polymer, a block copolymer in which a conjugate portion and a non-conjugate portion are present together, and the like, but are not limited thereto.

Electron Blocking Layer

The electron blocking layer is a layer provided between the hole transport layer and the light emitting layer in order to prevent the electrons injected in the cathode from being transferred to the hole transport layer without being recombined in the light emitting layer, which can also be referred to as an electron inhibition layer or an electron stopping layer. The electron blocking layer is preferably a material having a smaller electron affinity than the electron transport layer.

Light Emitting Layer

The light emitting layer used in the present disclosure is a layer that can emit light in the visible light region by combining holes and electrons transported from the anode and the cathode. Generally, the light emitting layer includes a host material and a dopant material, and in the present disclosure, the compound of Chemical Formula 1 and the compound of Chemical Formula 2 are included as a host

Preferably, the compound of Chemical Formula 1 includes at least one deuterium substituent.

Preferably, Ar₁ and Ar₂ can be each independently a substituted or unsubstituted C₆₋₂₀ aryl, or a substituted or unsubstituted C₂₋₂₀ heteroaryl containing at least one selected from the group consisting of N, O and S.

More preferably, Ar₁ and Ar₂ can be each independently phenyl, triphenylsilyl phenyl, biphenylyl, terphenylyl, naphthyl, phenanthrenyl, dibenzofuranyl, or dibenzothiophenyl, and the hydrogens of Ar₁ and Ar₂ can be each independently unsubstituted or substituted with deuterium.

Most preferably, Ar₁ and Ar₂ can be each independently any one selected from the group consisting of:

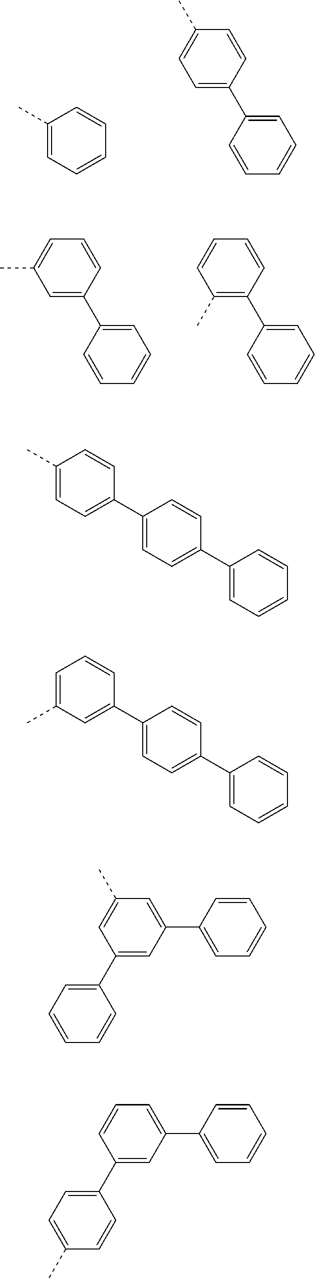

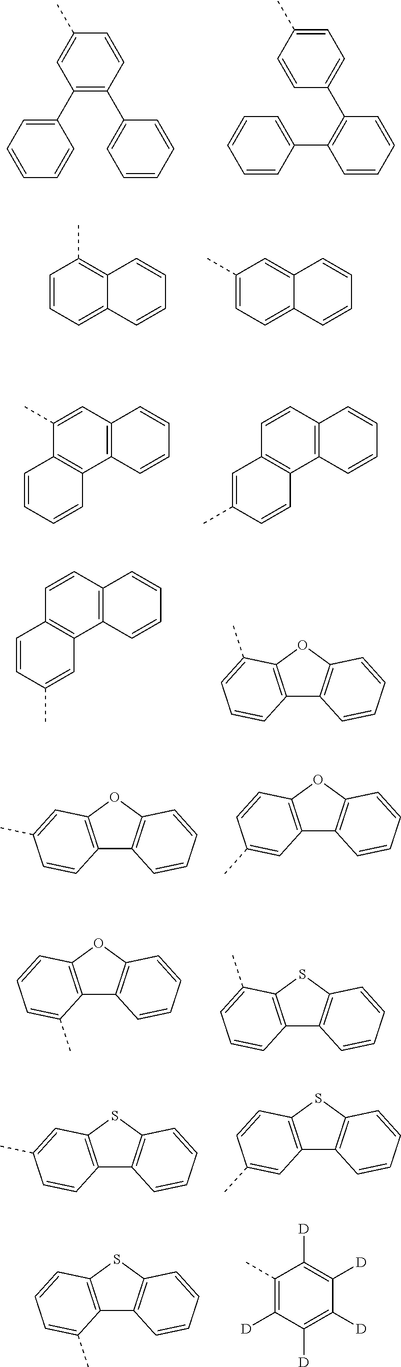

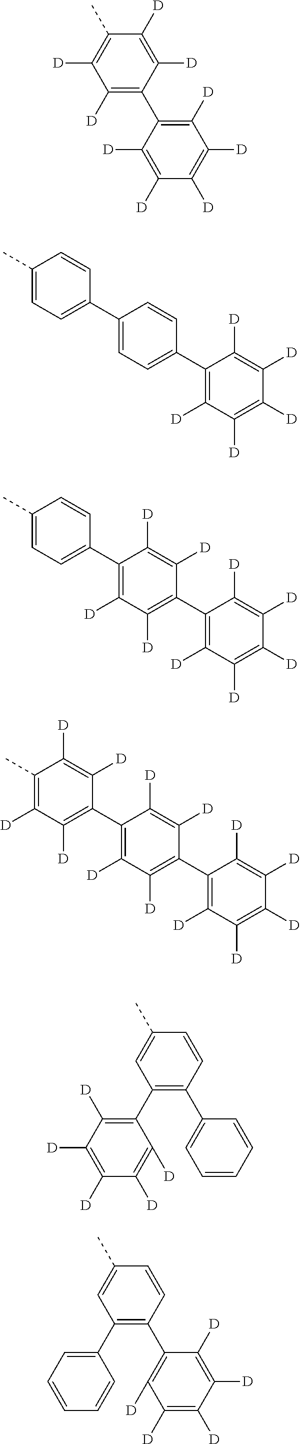

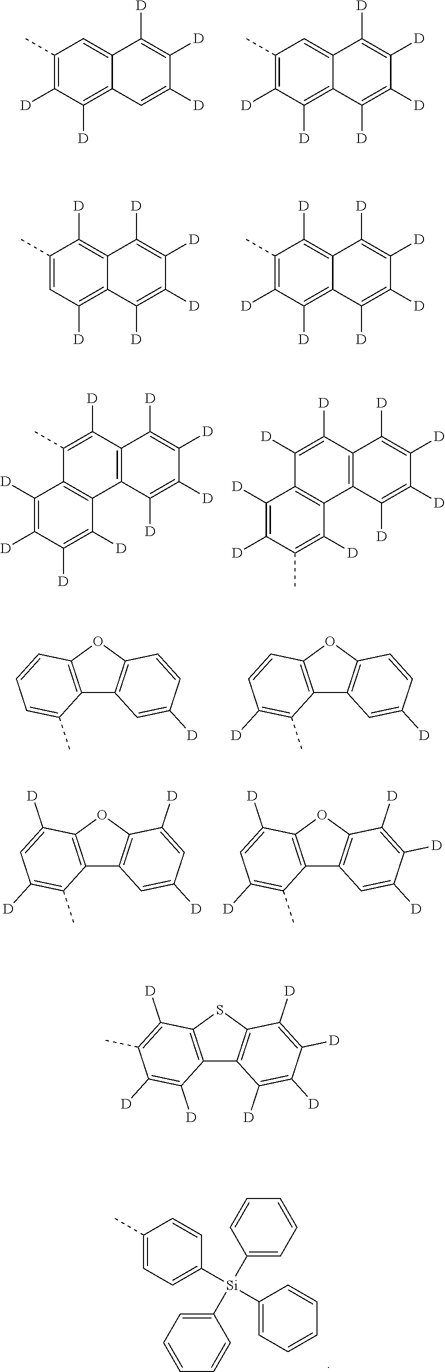

Preferably, L₁ to L₃ can be each independently a single bond or a substituted or unsubstituted C₆₋₂₀ arylene.

More preferably, L₁ to L₃ can be each independently a single bond, phenylene, biphenyldiyl, or naphthalenediyl, and the hydrogens of L₁ to L₃ can be each independently unsubstituted or substituted with deuterium.

More preferably, L₁ to L₃ can be each independently a single bond, or any one selected from the group consisting of:

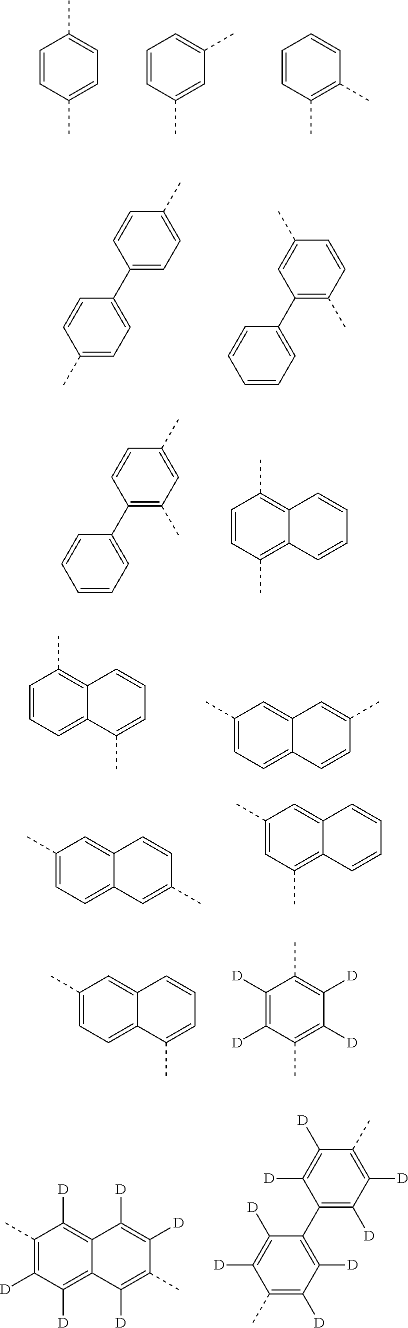

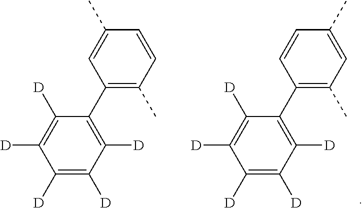

In this case, a represents the number of R₁, and when a is 2 or more, two or more R₁s can be the same or different from each other.

Preferably, a can be an integer of 1 to 7.

Representative examples of the first compound of Chemical Formula 1 are as follows:

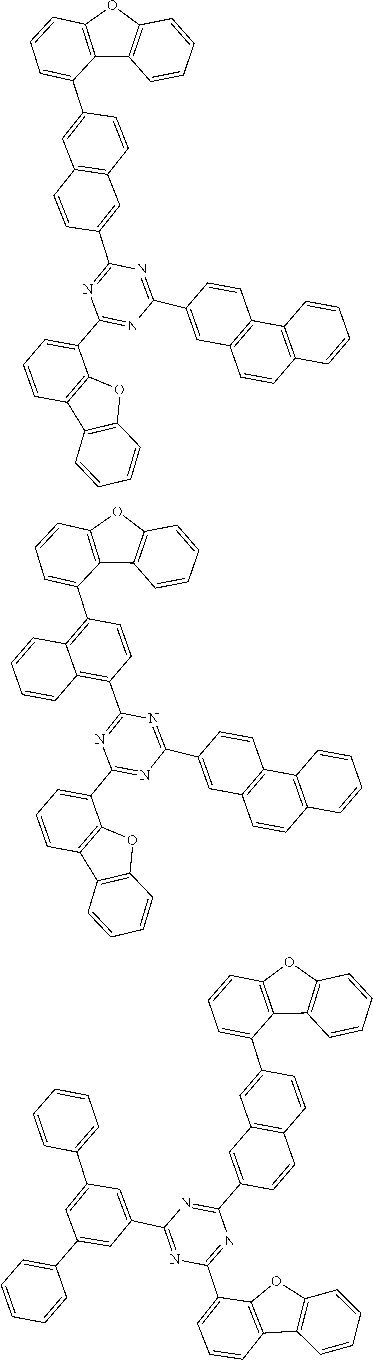

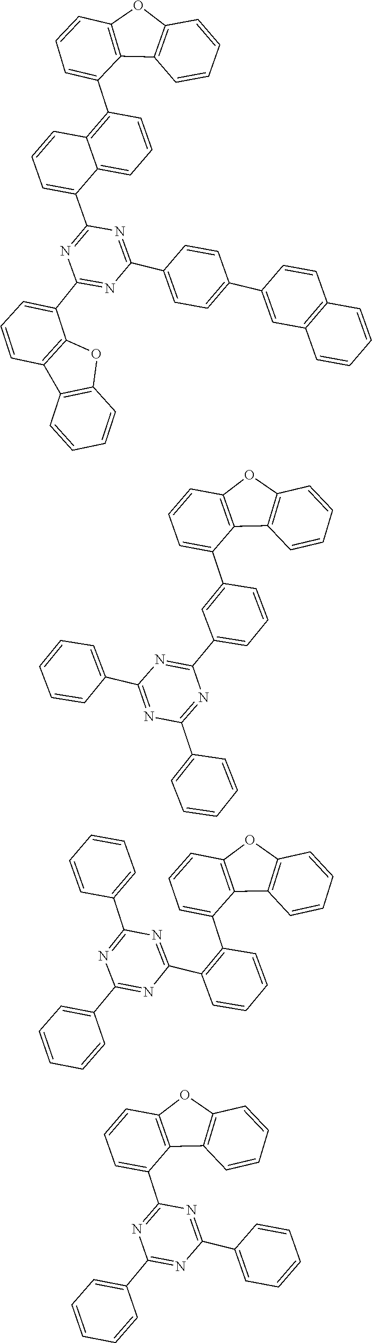

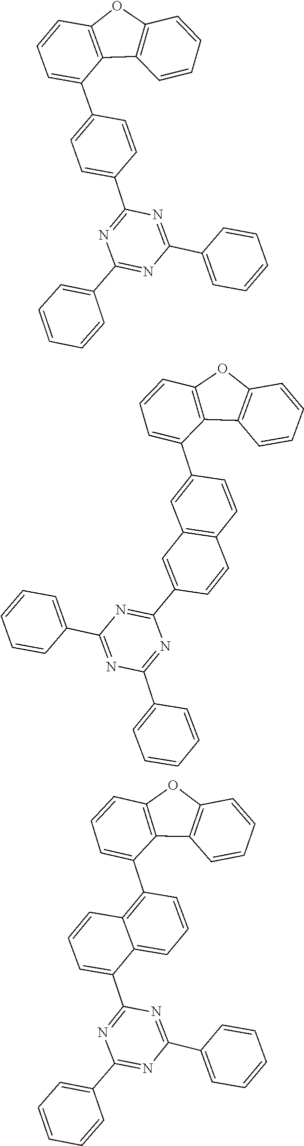

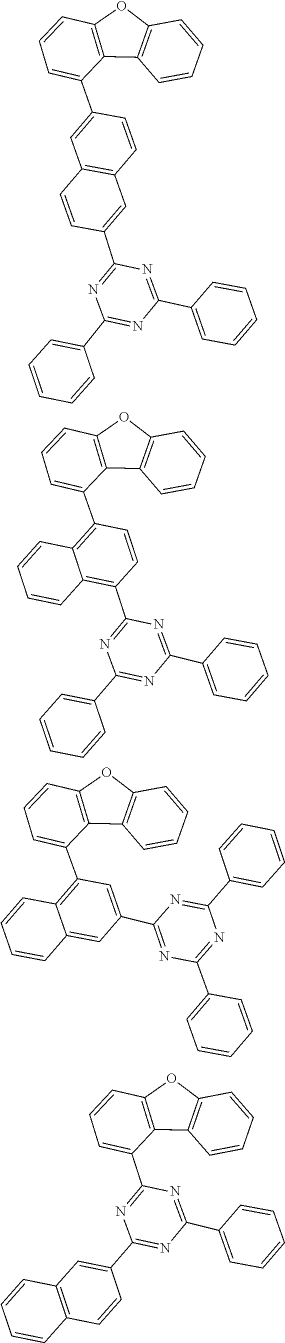

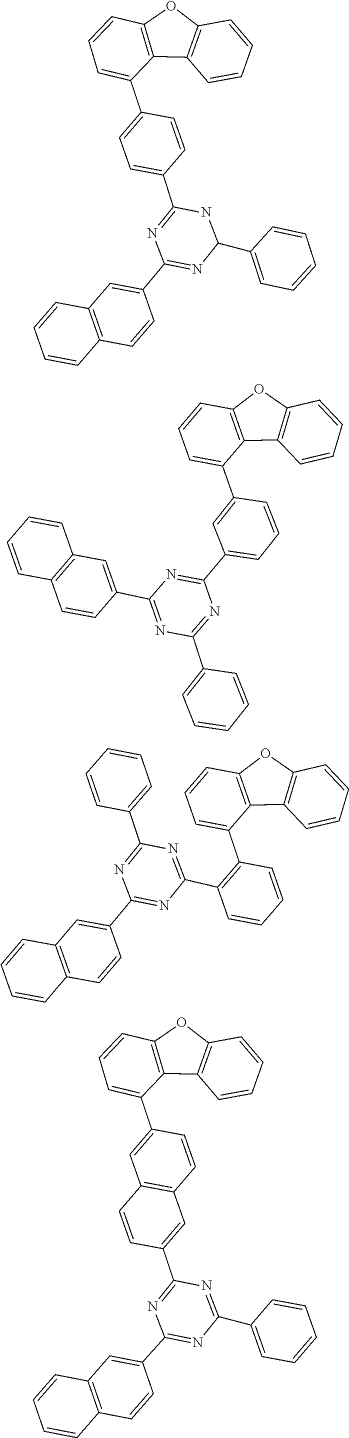

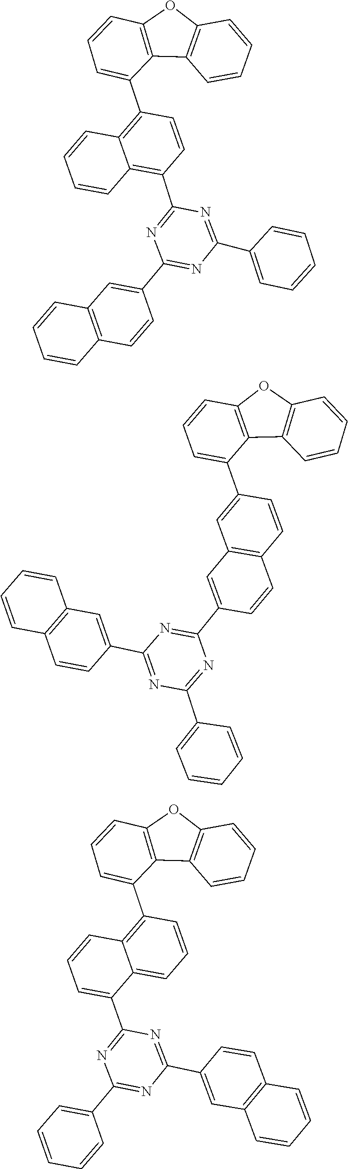

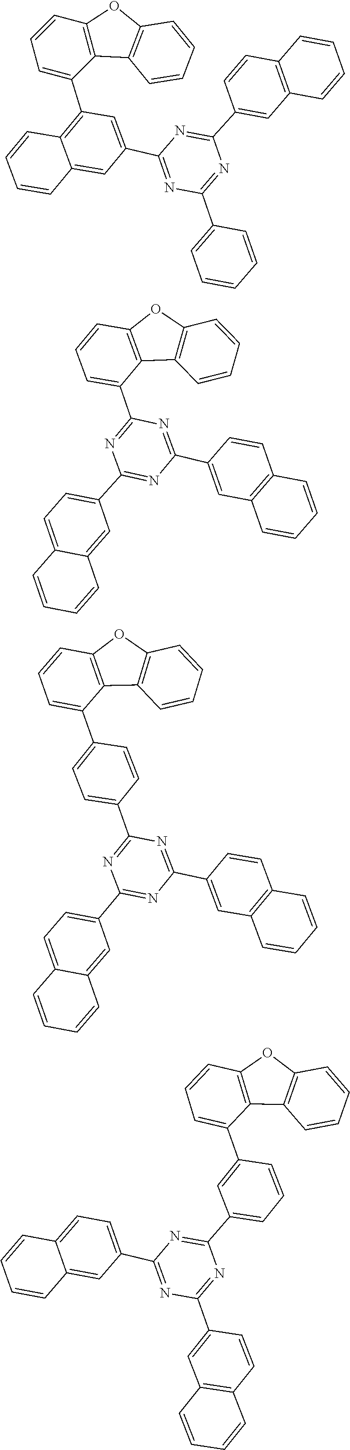

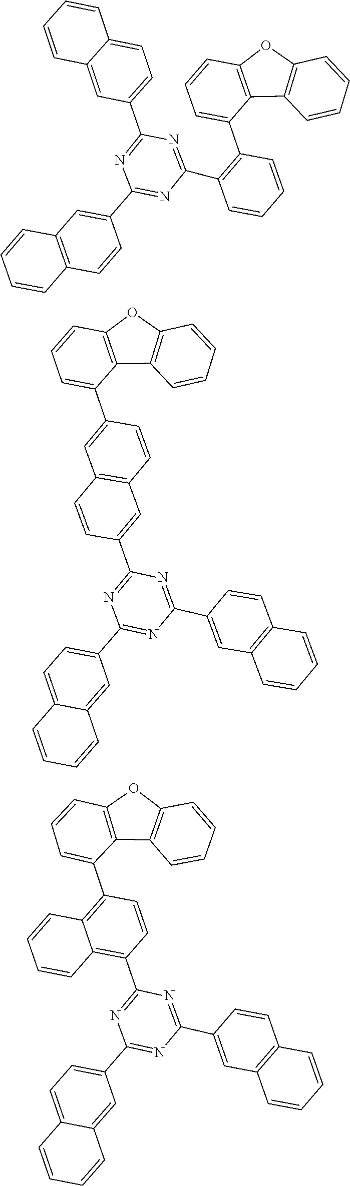

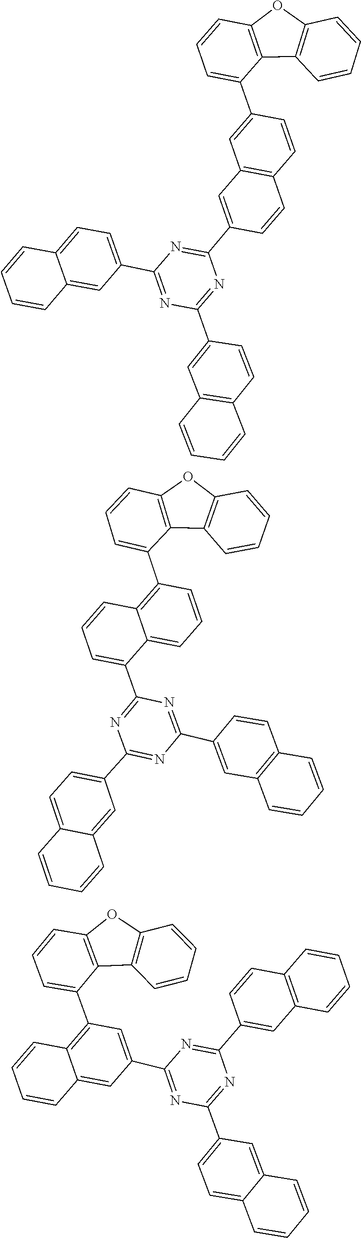

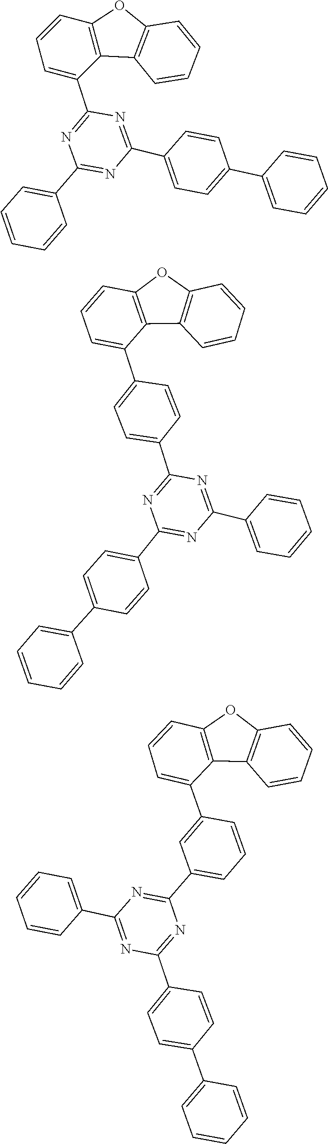 [SMILES: c1ccc(-c2ccc(-c3nc(-c4ccccc4)nc(-c4cccc5oc6ccccc6c45)n3)cc2)cc1 ; c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4cccc(-c5cccc6oc7ccccc7c56)c4)n3)cc2)cc1]

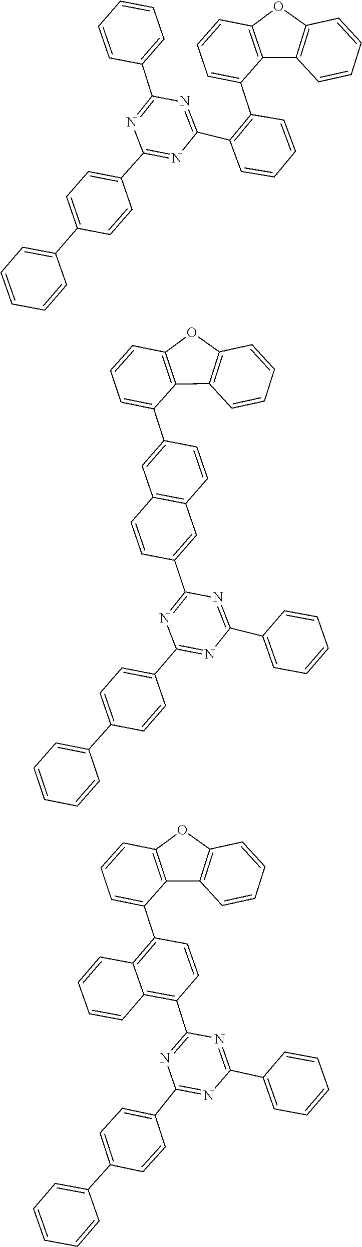 [SMILES: c1ccc(-c2ccc(-c3nc(-c4ccccc4)nc(-c4ccccc4-c4cccc5oc6ccccc6c45)n3)cc2)cc1 ; c1ccc(-c2ccc(-c3nc(-c4ccccc4)nc(-c4ccc5cc(-c6cccc7oc8ccccc8c67)ccc5c4)n3)cc2)cc1 ; c1ccc(-c2ccc(-c3nc(-c4ccccc4)nc(-c4ccc(-c5cccc6oc7ccccc7c56)c5ccccc45)n3)cc2)cc1]

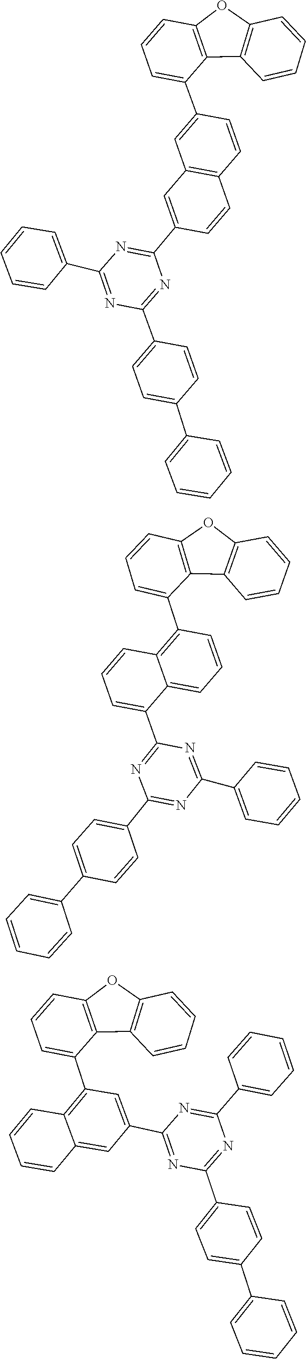

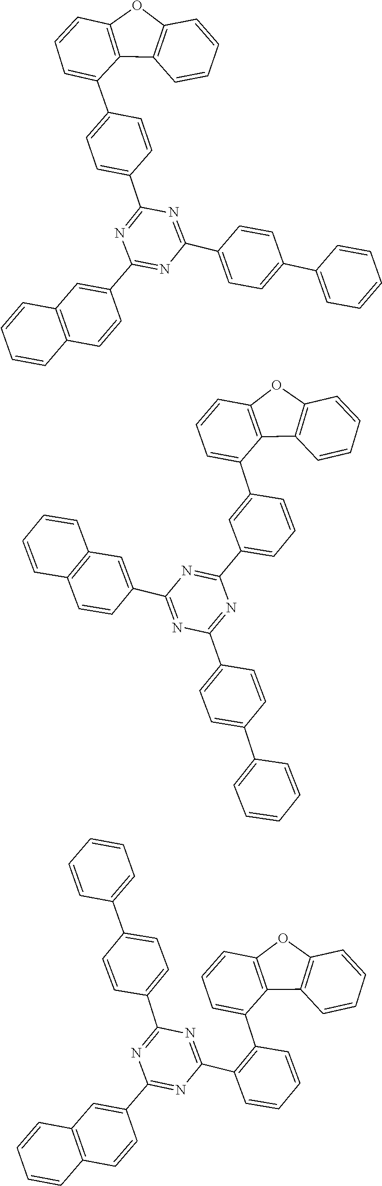 [SMILES: c1ccc(-c2ccc(-c3nc(-c4ccc(-c5cccc6oc7ccccc7c56)cc4)nc(-c4ccc5ccccc5c4)n3)cc2)cc1 ; c1ccc(-c2ccc(-c3nc(-c4cccc(-c5cccc6oc7ccccc7c56)c4)nc(-c4ccc5ccccc5c4)n3)cc2)cc1 ; c1ccc(-c2ccc(-c3nc(-c4ccc5ccccc5c4)nc(-c4ccccc4-c4cccc5oc6ccccc6c45)n3)cc2)cc1]

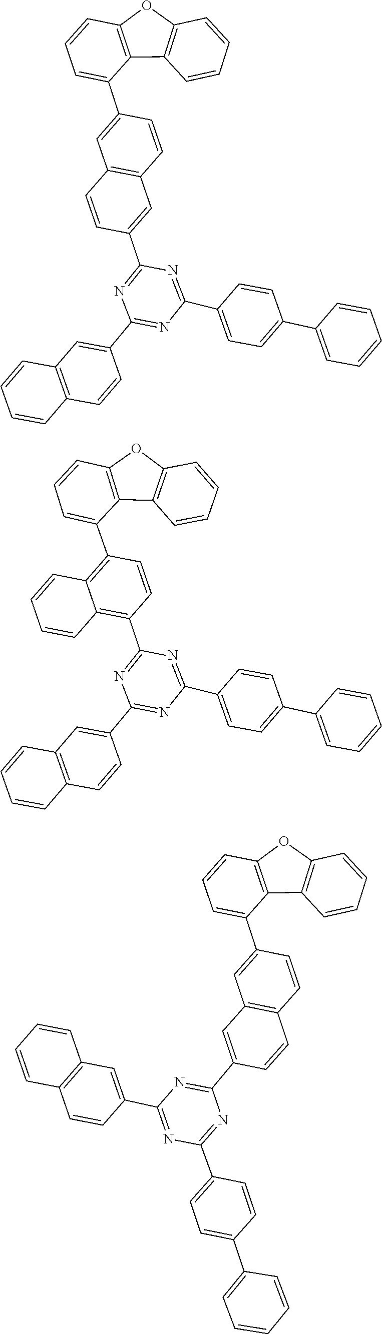 [SMILES: c1ccc(-c2ccc(-c3nc(-c4ccc5ccccc5c4)nc(-c4ccc5cc(-c6cccc7oc8ccccc8c67)ccc5c4)n3)cc2)cc1 ; c1ccc(-c2ccc(-c3nc(-c4ccc5ccccc5c4)nc(-c4ccc(-c5cccc6oc7ccccc7c56)c5ccccc45)n3)cc2)cc1 ; c1ccc(-c2ccc(-c3nc(-c4ccc5ccccc5c4)nc(-c4ccc5ccc(-c6cccc7oc8ccccc8c67)cc5c4)n3)cc2)cc1]

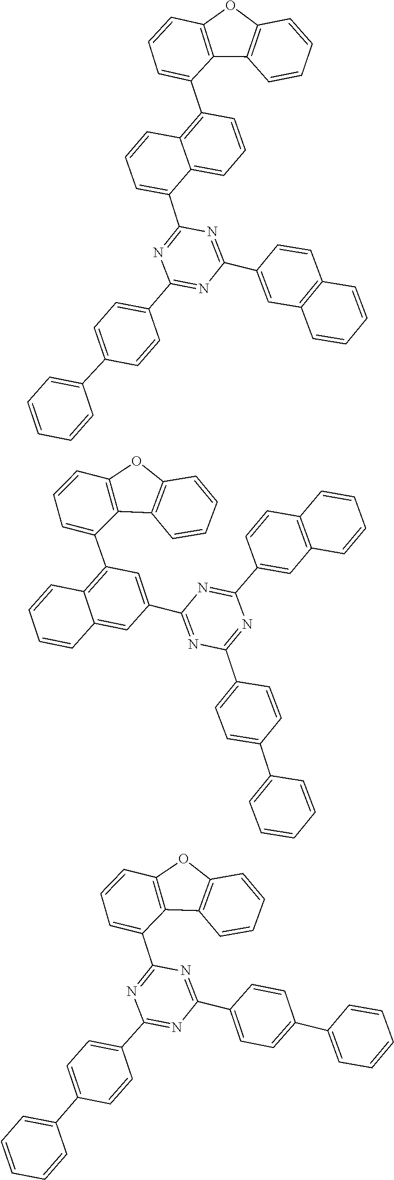

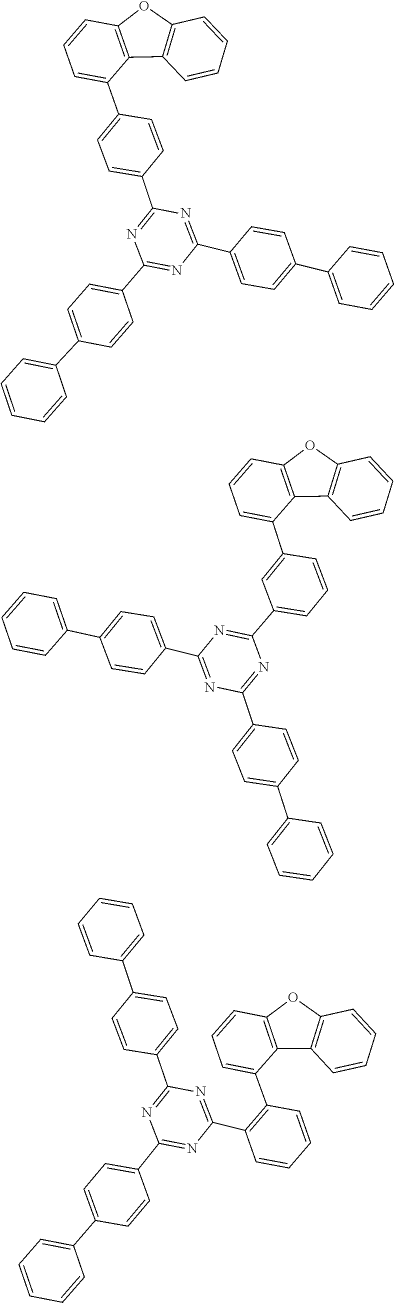 [SMILES: c1ccc(-c2ccc(-c3nc(-c4ccc(-c5ccccc5)cc4)nc(-c4ccc(-c5cccc6oc7ccccc7c56)cc4)n3)cc2)cc1 ; c1ccc(-c2ccc(-c3nc(-c4ccc(-c5ccccc5)cc4)nc(-c4cccc(-c5cccc6oc7ccccc7c56)c4)n3)cc2)cc1 ; c1ccc(-c2ccc(-c3nc(-c4ccc(-c5ccccc5)cc4)nc(-c4ccccc4-c4cccc5oc6ccccc6c45)n3)cc2)cc1]

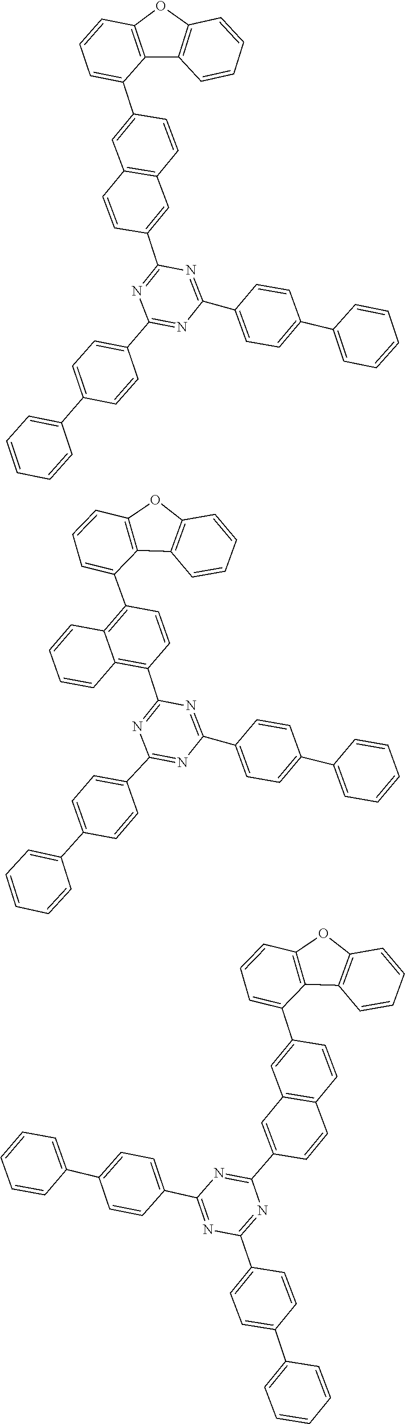 [SMILES: c1ccc(-c2ccc(-c3nc(-c4ccc(-c5ccccc5)cc4)nc(-c4ccc5cc(-c6cccc7oc8ccccc8c67)ccc5c4)n3)cc2)cc1 ; c1ccc(-c2ccc(-c3nc(-c4ccc(-c5ccccc5)cc4)nc(-c4ccc(-c5cccc6oc7ccccc7c56)c5ccccc45)n3)cc2)cc1 ; c1ccc(-c2ccc(-c3nc(-c4ccc(-c5ccccc5)cc4)nc(-c4ccc5ccc(-c6cccc7oc8ccccc8c67)cc5c4)n3)cc2)cc1]

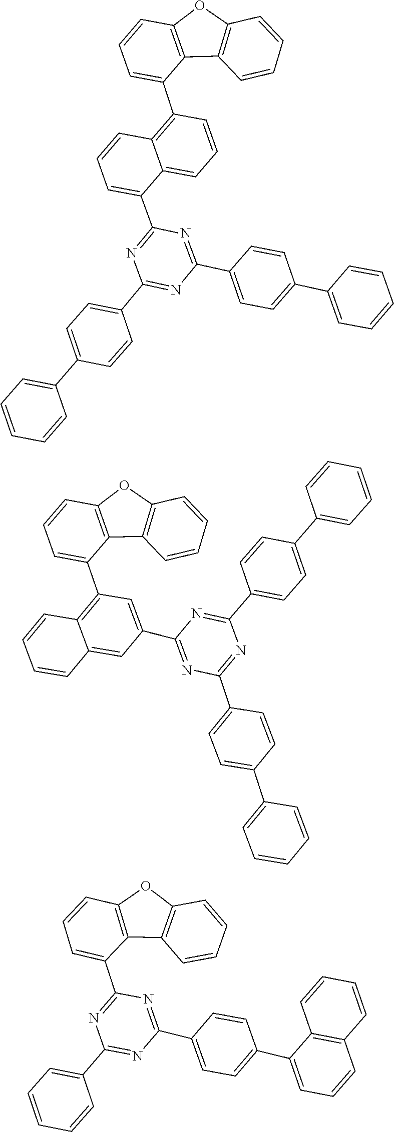 [SMILES: c1ccc(-c2ccc(-c3nc(-c4ccc(-c5ccccc5)cc4)nc(-c4cccc5c(-c6cccc7oc8ccccc8c67)cccc45)n3)cc2)cc1 ; c1ccc(-c2ccc(-c3nc(-c4ccc(-c5ccccc5)cc4)nc(-c4cc(-c5cccc6oc7ccccc7c56)c5ccccc5c4)n3)cc2)cc1 ; c1ccc(-c2nc(-c3ccc(-c4cccc5ccccc45)cc3)nc(-c3cccc4oc5ccccc5c34)n2)cc1]

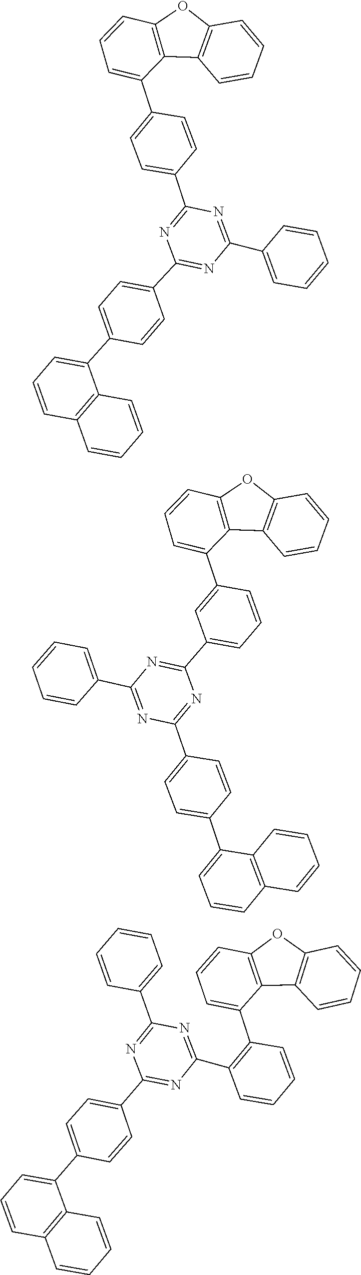

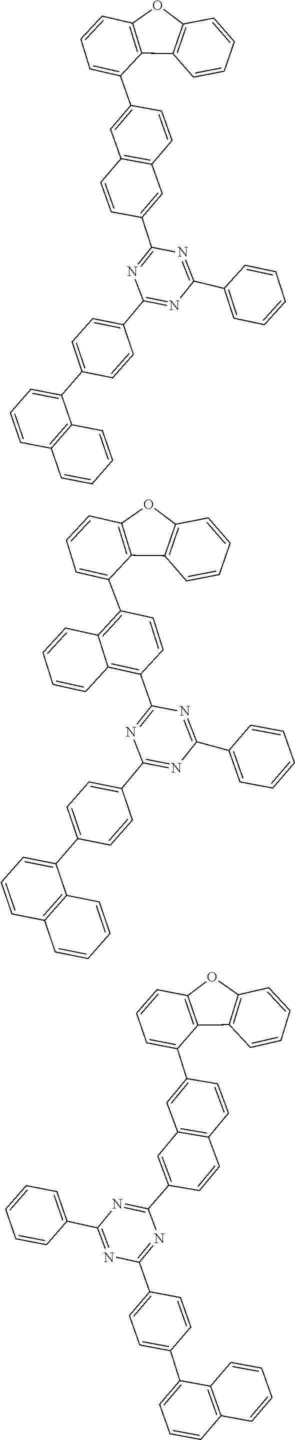

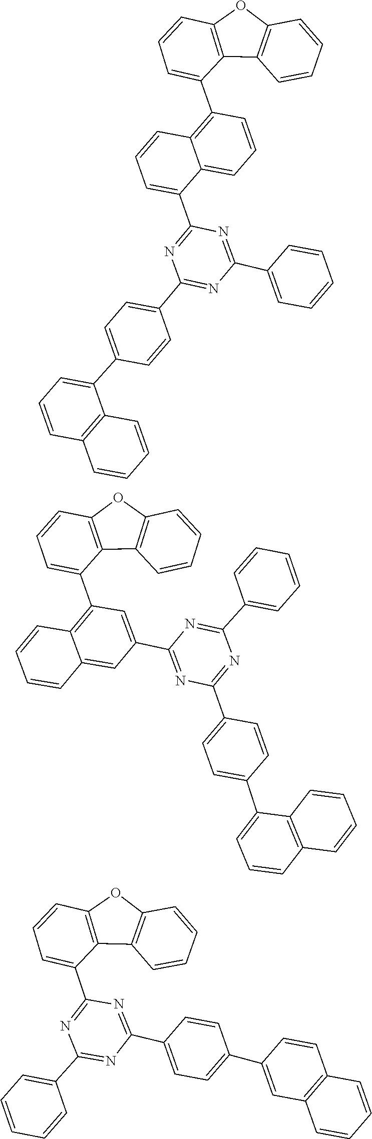

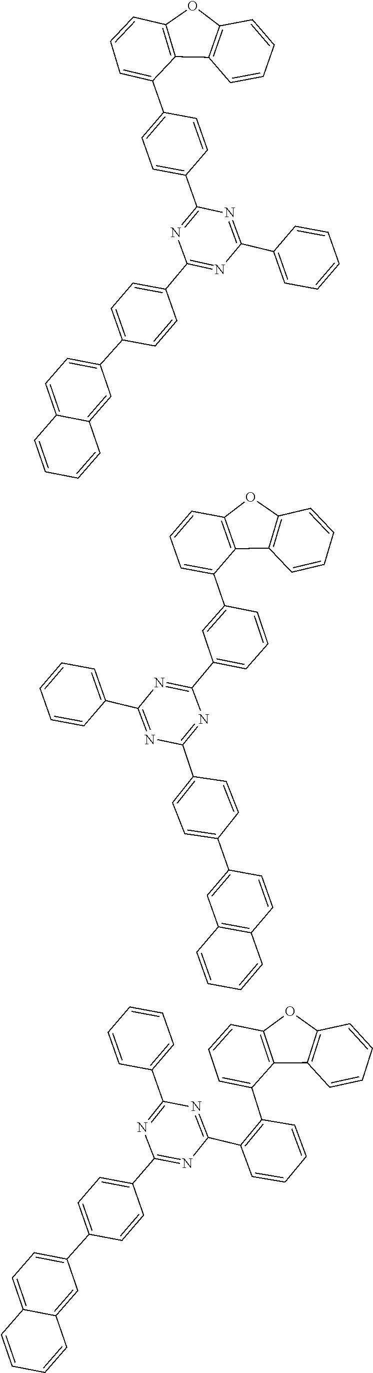

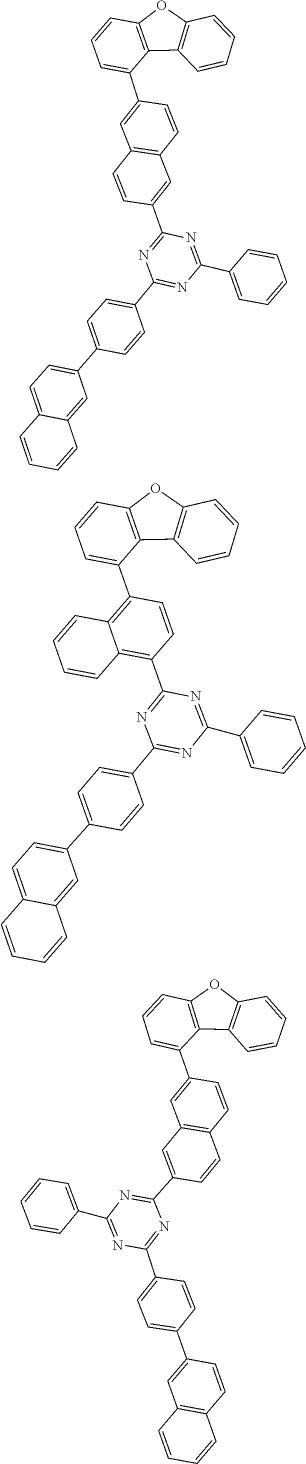 [SMILES: c1ccc(-c2nc(-c3ccc(-c4ccc5ccccc5c4)cc3)nc(-c3ccc4cc(-c5cccc6oc7ccccc7c56)ccc4c3)n2)cc1 ; c1ccc(-c2nc(-c3ccc(-c4ccc5ccccc5c4)cc3)nc(-c3ccc(-c4cccc5oc6ccccc6c45)c4ccccc34)n2)cc1 ; c1ccc(-c2nc(-c3ccc(-c4ccc5ccccc5c4)cc3)nc(-c3ccc4ccc(-c5cccc6oc7ccccc7c56)cc4c3)n2)cc1]

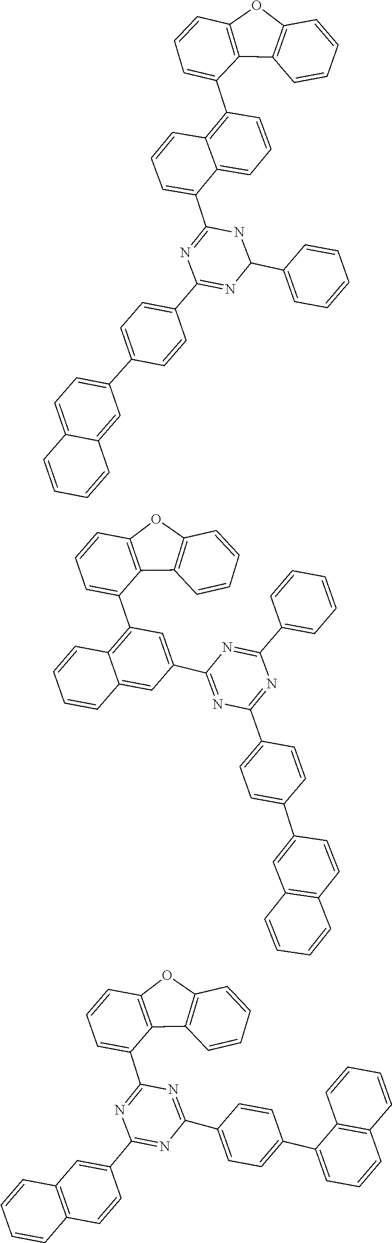

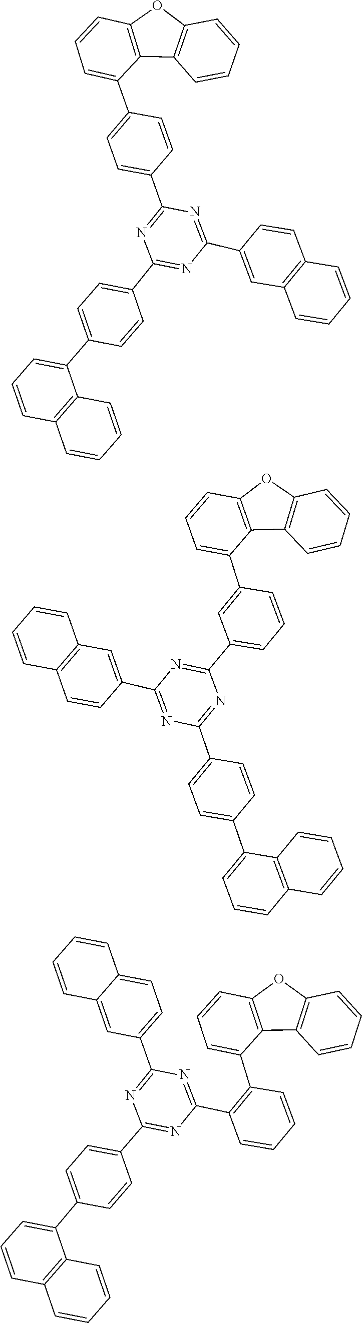 [SMILES: c1ccc2cc(-c3nc(-c4ccc(-c5cccc6ccccc56)cc4)nc(-c4ccc(-c5cccc6oc7ccccc7c56)cc4)n3)ccc2c1 ; c1cc(-c2nc(-c3ccc(-c4cccc5ccccc45)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4cccc5ccccc45)cc3)nc(-c3ccc4ccccc4c3)n2)c1]

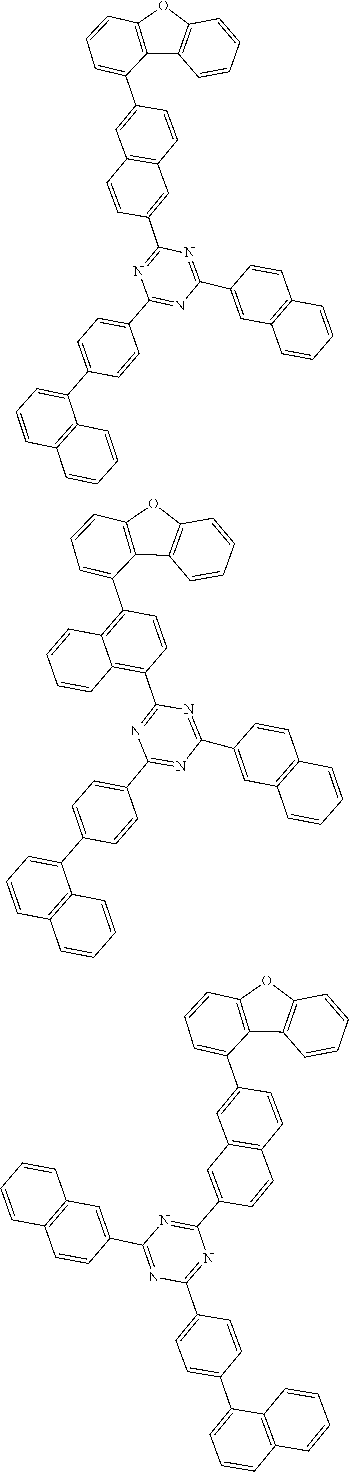

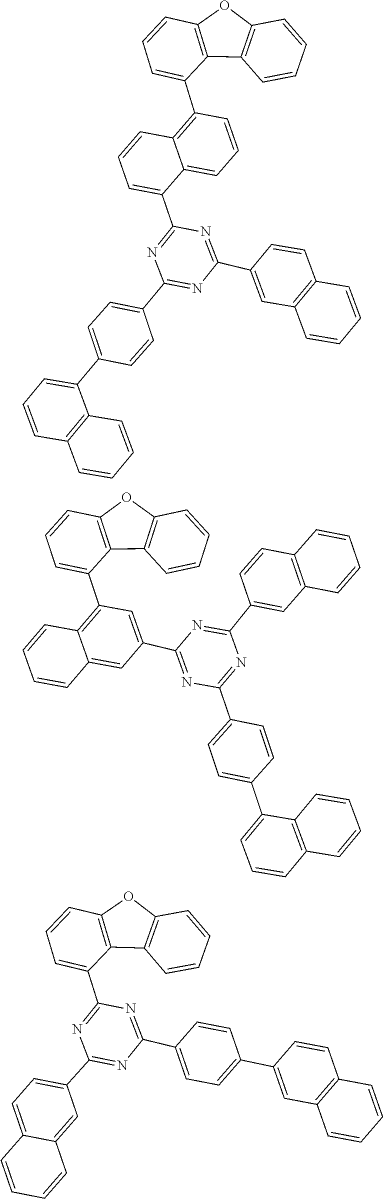

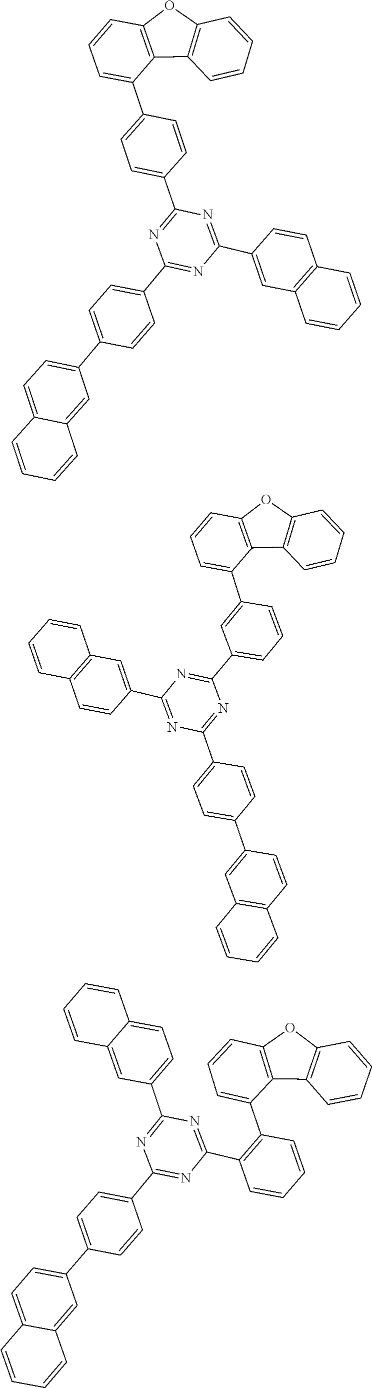 [SMILES: c1ccc2cc(-c3ccc(-c4nc(-c5ccc(-c6cccc7oc8ccccc8c67)cc5)nc(-c5ccc6ccccc6c5)n4)cc3)ccc2c1 ; c1cc(-c2nc(-c3ccc(-c4ccc5ccccc5c4)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4ccc5ccccc5c4)cc3)nc(-c3ccc4ccccc4c3)n2)c1]

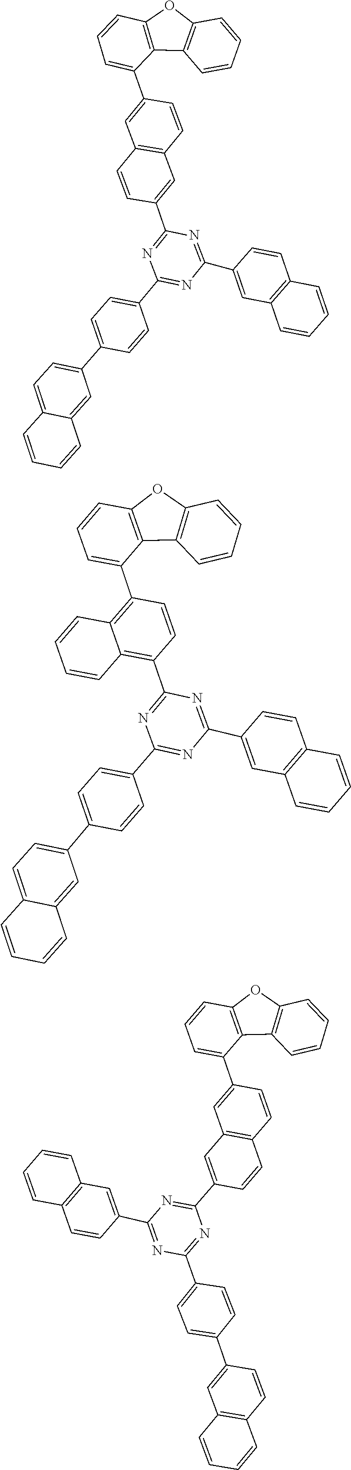 [SMILES: c1ccc2cc(-c3ccc(-c4nc(-c5ccc6ccccc6c5)nc(-c5ccc6cc(-c7cccc8oc9ccccc9c78)ccc6c5)n4)cc3)ccc2c1 ; c1ccc2cc(-c3ccc(-c4nc(-c5ccc6ccccc6c5)nc(-c5ccc(-c6cccc7oc8ccccc8c67)c6ccccc56)n4)cc3)ccc2c1 ; c1ccc2cc(-c3ccc(-c4nc(-c5ccc6ccccc6c5)nc(-c5ccc6ccc(-c7cccc8oc9ccccc9c78)cc6c5)n4)cc3)ccc2c1]

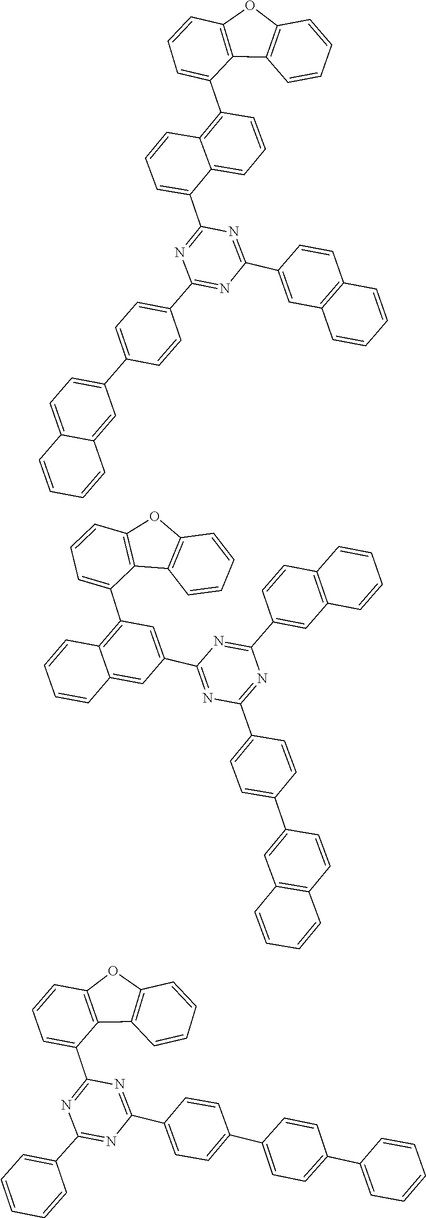

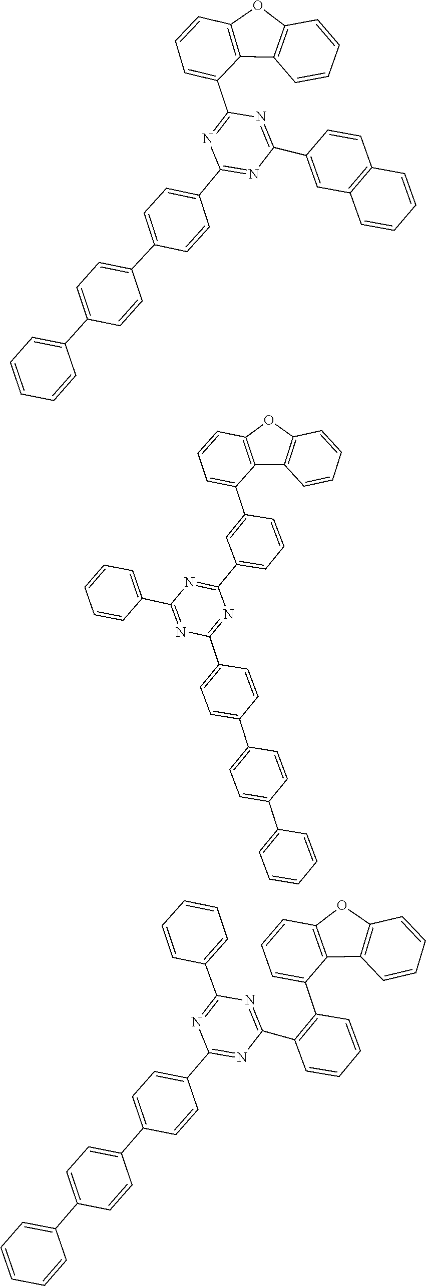

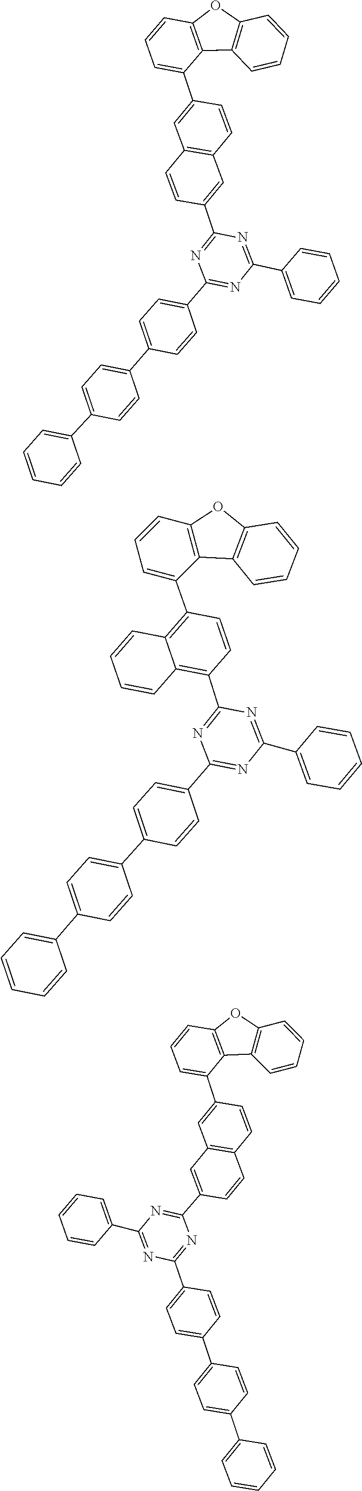 [SMILES: c1ccc(-c2ccc(-c3ccc(-c4nc(-c5ccccc5)nc(-c5ccc6cc(-c7cccc8oc9ccccc9c78)ccc6c5)n4)cc3)cc2)cc1 ; c1ccc(-c2ccc(-c3ccc(-c4nc(-c5ccccc5)nc(-c5ccc(-c6cccc7oc8ccccc8c67)c6ccccc56)n4)cc3)cc2)cc1 ; c1ccc(-c2ccc(-c3ccc(-c4nc(-c5ccccc5)nc(-c5ccc6ccc(-c7cccc8oc9ccccc9c78)cc6c5)n4)cc3)cc2)cc1]

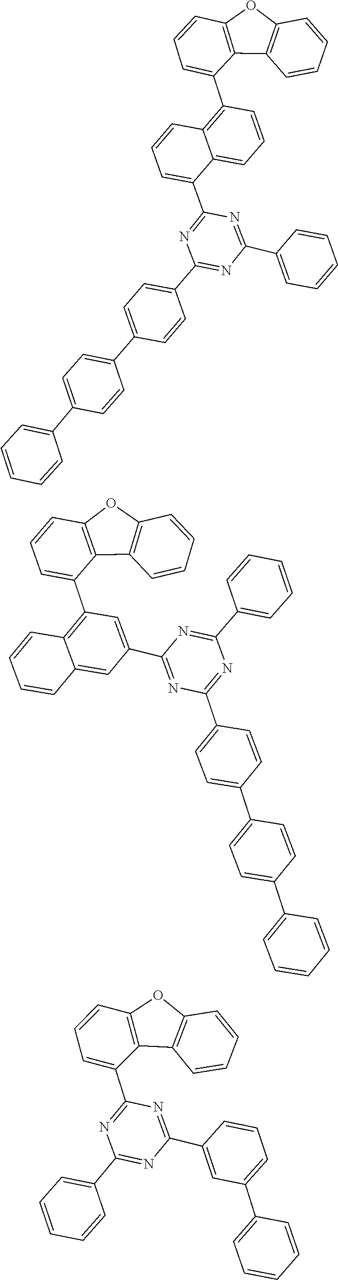

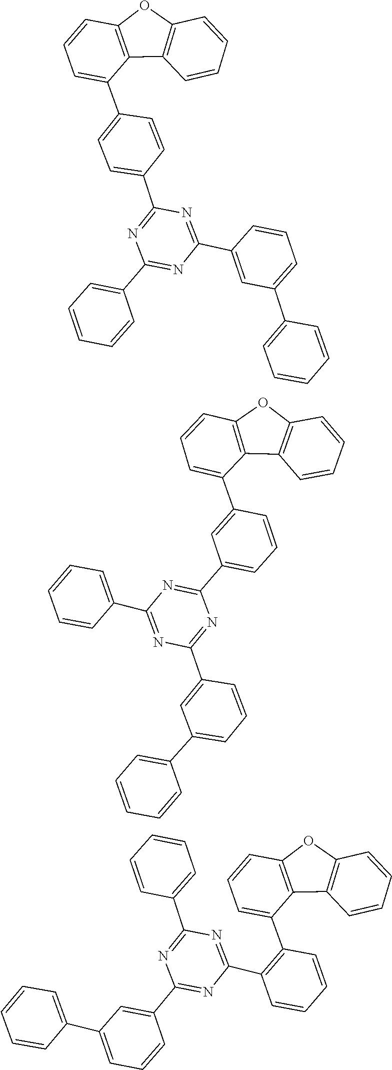

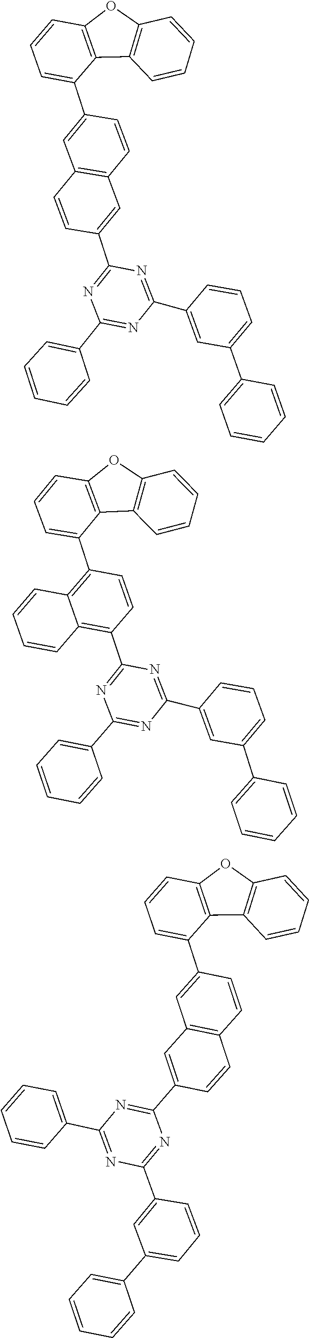

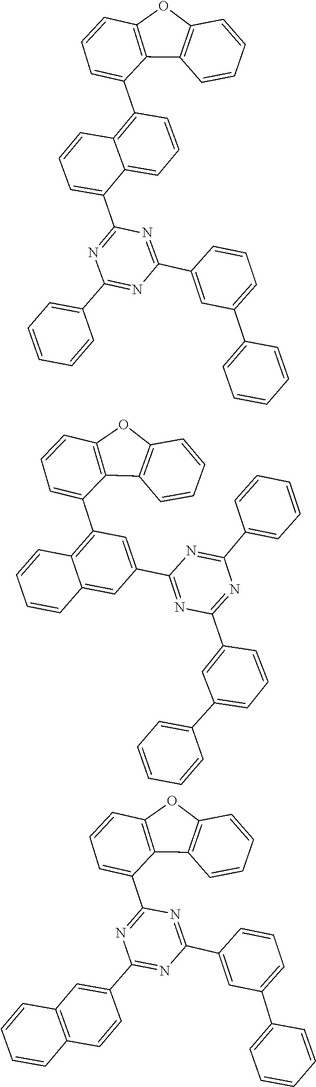

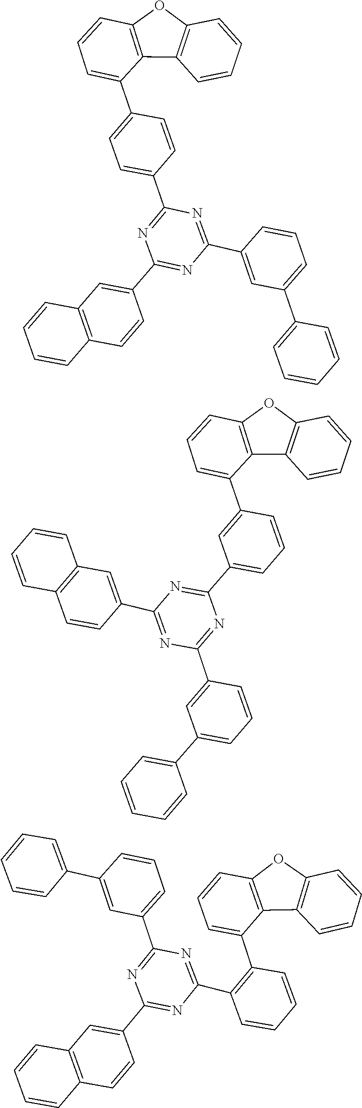

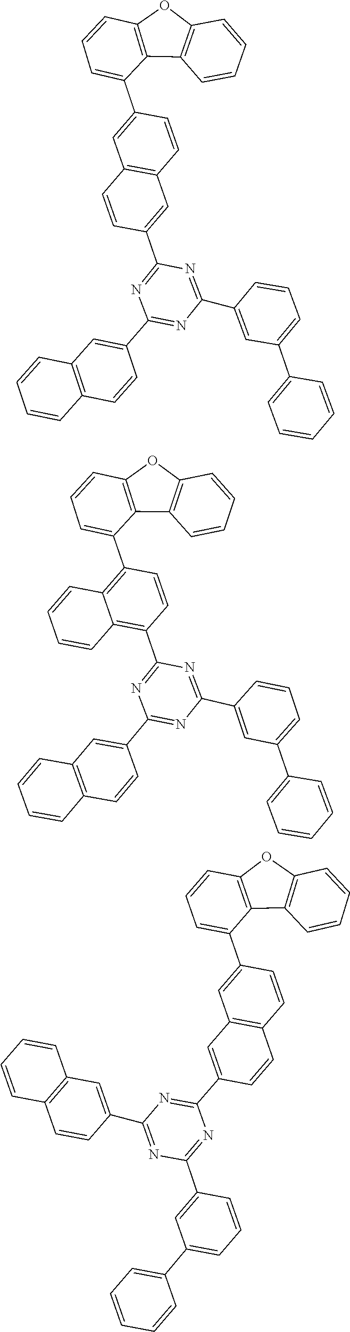

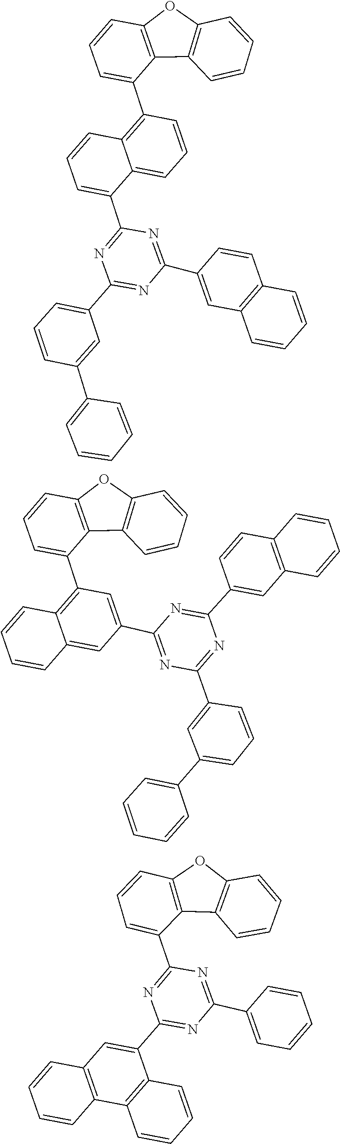

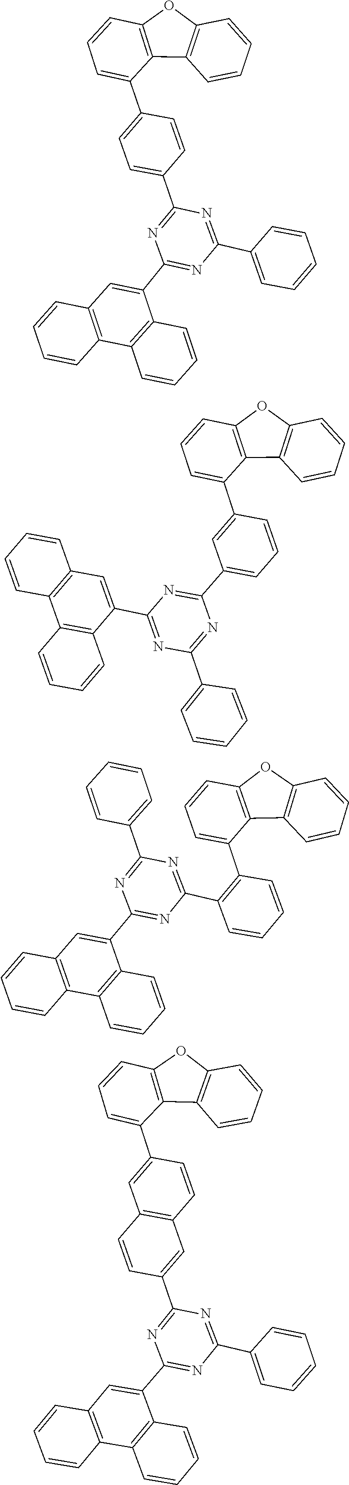

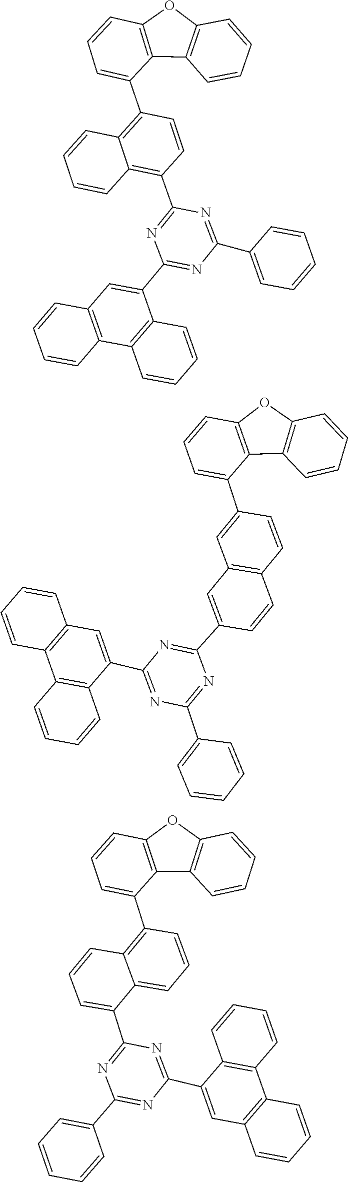

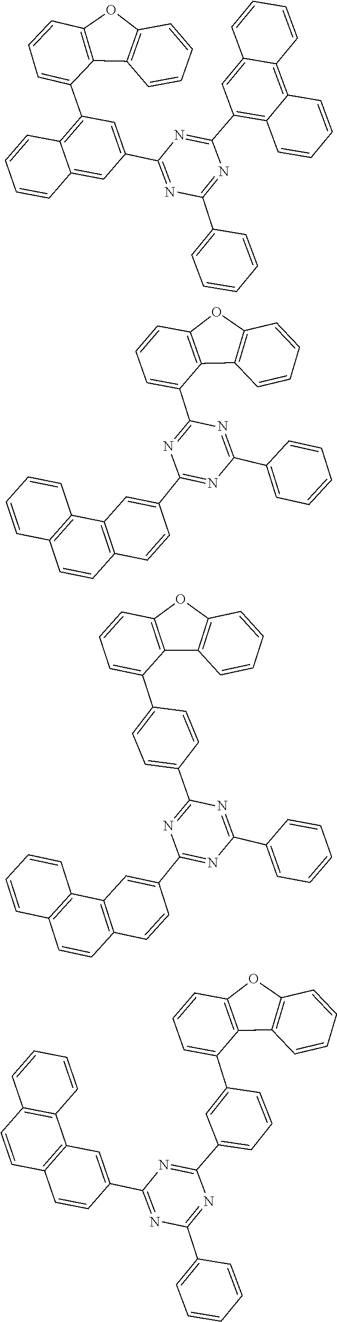

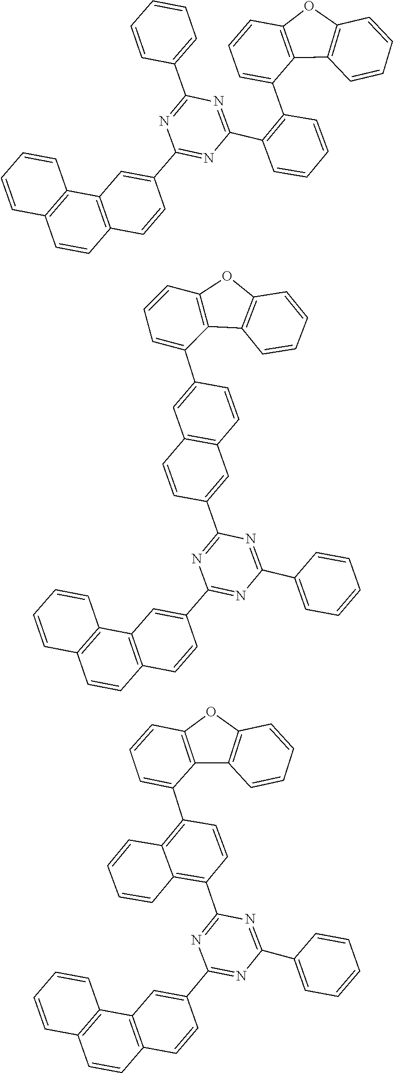

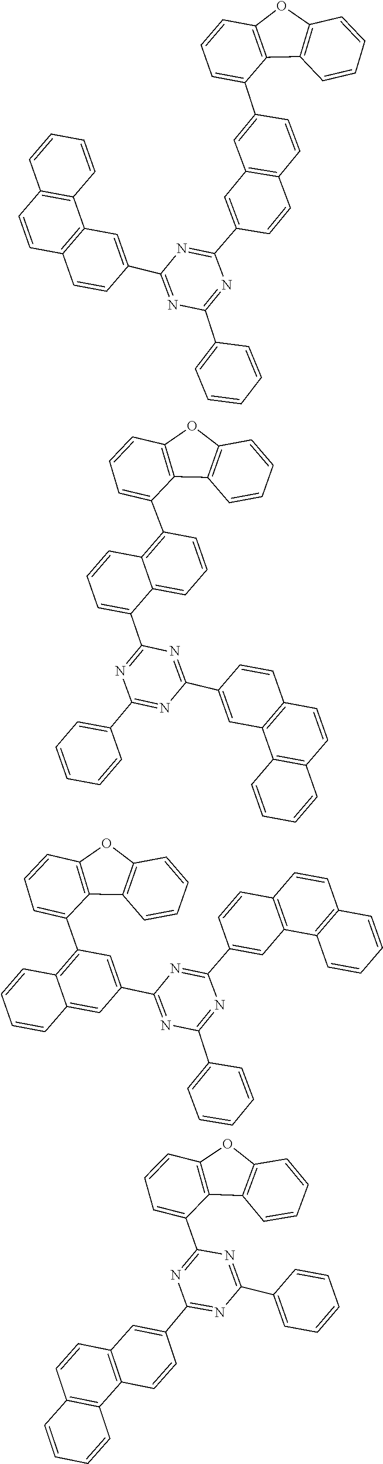

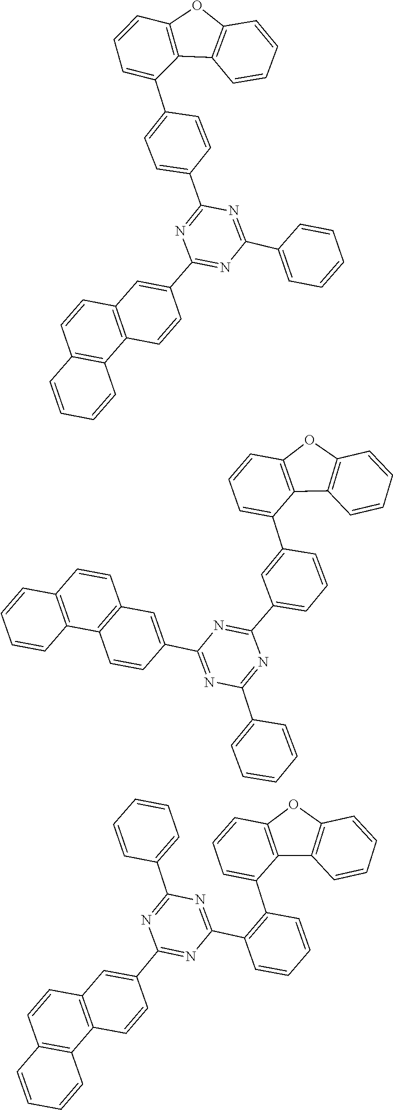

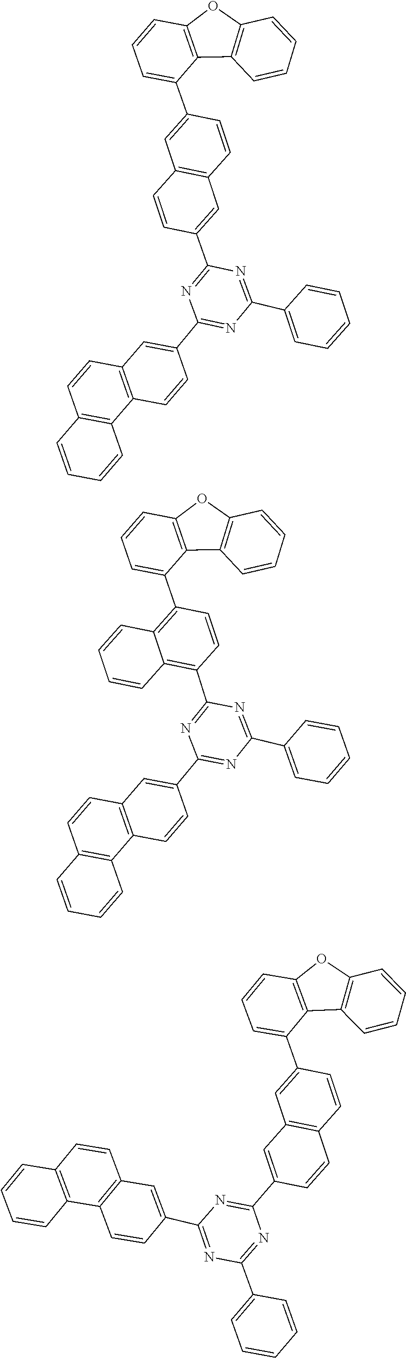 [SMILES: c1ccc(-c2nc(-c3ccc4cc(-c5cccc6oc7ccccc7c56)ccc4c3)nc(-c3ccc4c(ccc5ccccc54)c3)n2)cc1 ; c1ccc(-c2nc(-c3ccc4c(ccc5ccccc54)c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(ccc5ccccc54)c3)n2)cc1]

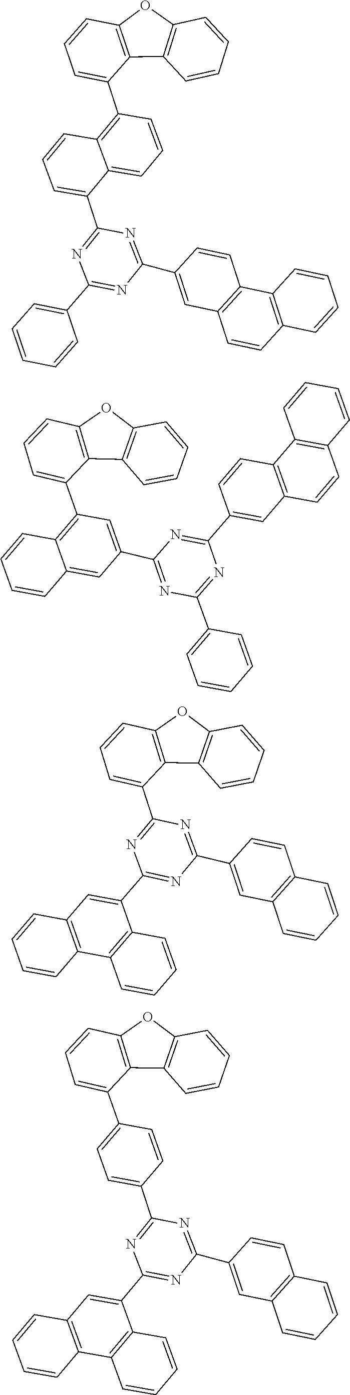

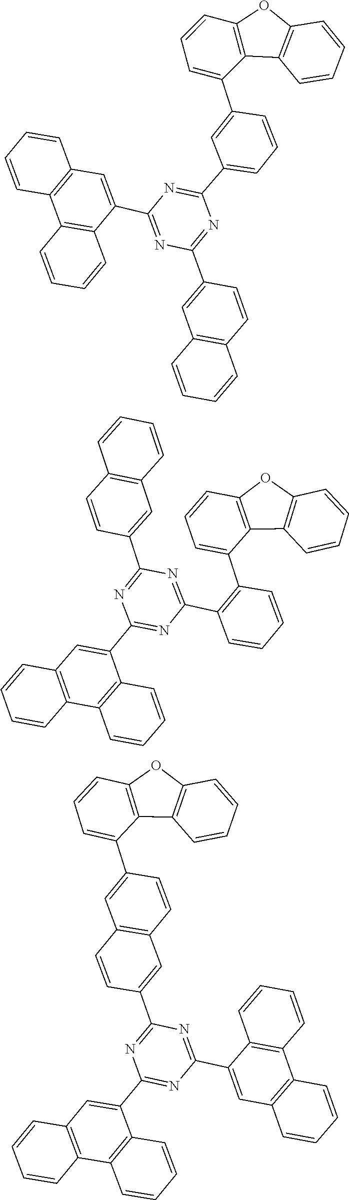

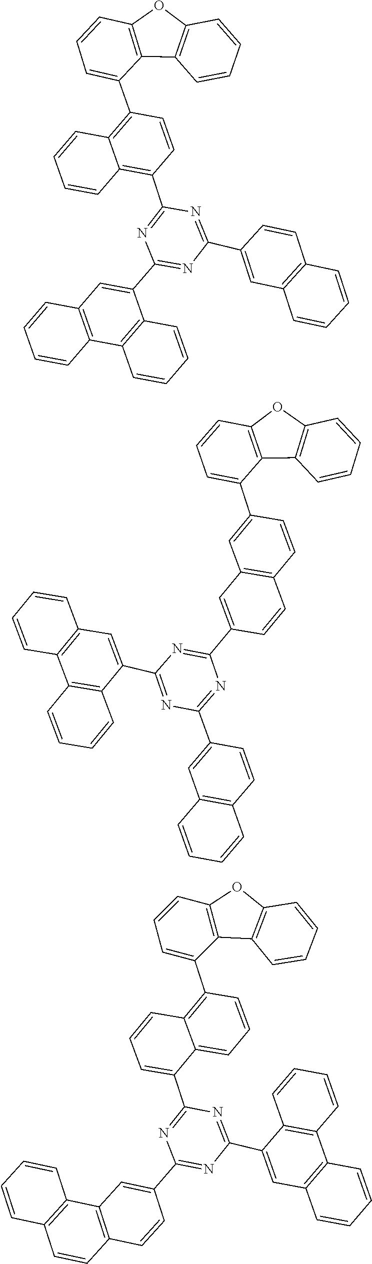

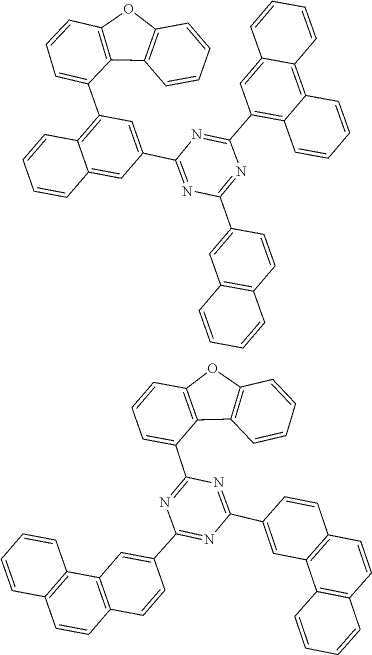

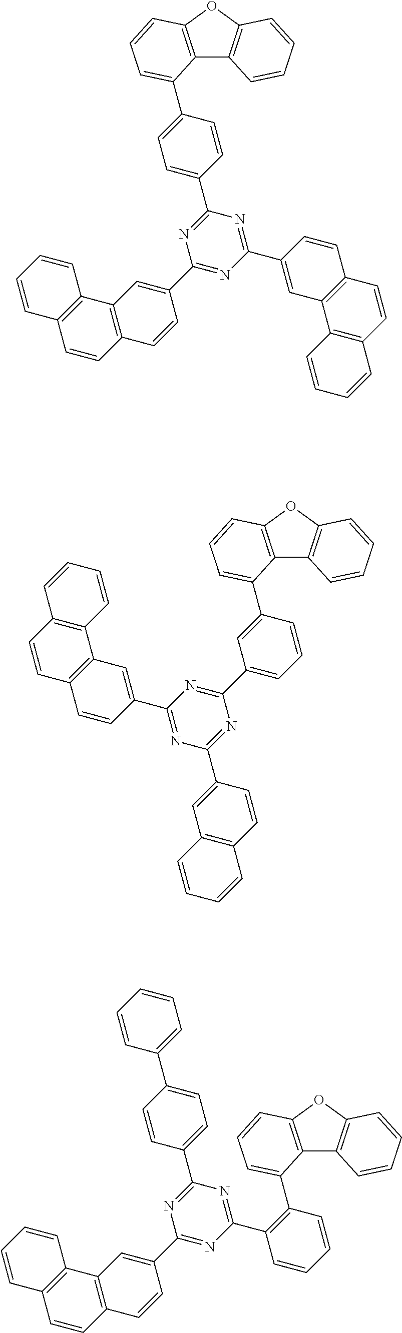 [SMILES: c1ccc2c(c1)ccc1ccc(-c3nc(-c4ccc(-c5cccc6oc7ccccc7c56)cc4)nc(-c4ccc5ccc6ccccc6c5c4)n3)cc12 ; c1cc(-c2nc(-c3ccc4ccccc4c3)nc(-c3ccc4ccc5ccccc5c4c3)n2)cc(-c2cccc3oc4ccccc4c23)c1 ; c1ccc(-c2ccc(-c3nc(-c4ccc5ccc6ccccc6c5c4)nc(-c4ccccc4-c4cccc5oc6ccccc6c45)n3)cc2)cc1]

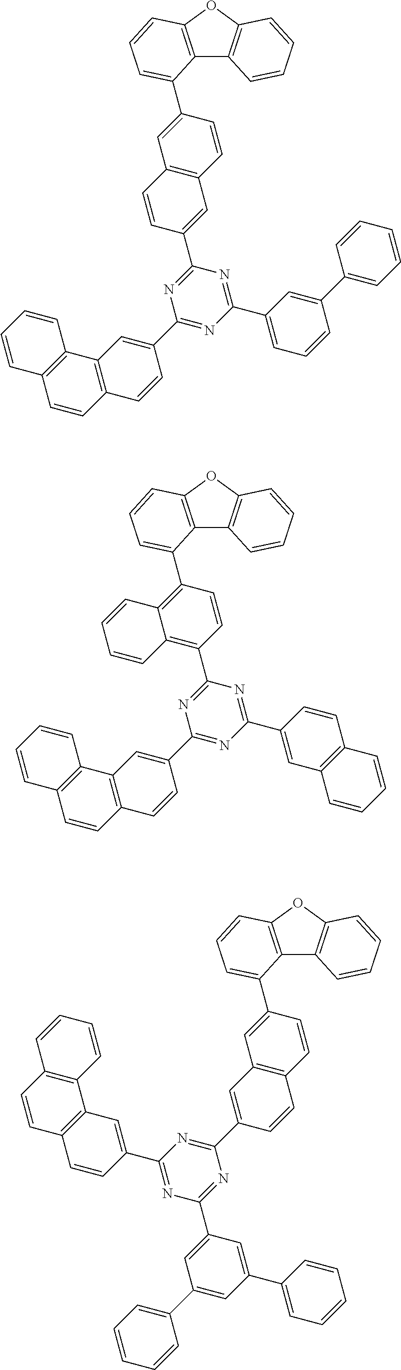 [SMILES: c1ccc(-c2cccc(-c3nc(-c4ccc5cc(-c6cccc7oc8ccccc8c67)ccc5c4)nc(-c4ccc5ccc6ccccc6c5c4)n3)c2)cc1 ; c1ccc2cc(-c3nc(-c4ccc5ccc6ccccc6c5c4)nc(-c4ccc(-c5cccc6oc7ccccc7c56)c5ccccc45)n3)ccc2c1 ; c1ccc(-c2cc(-c3ccccc3)cc(-c3nc(-c4ccc5ccc(-c6cccc7oc8ccccc8c67)cc5c4)nc(-c4ccc5ccc6ccccc6c5c4)n3)c2)cc1]

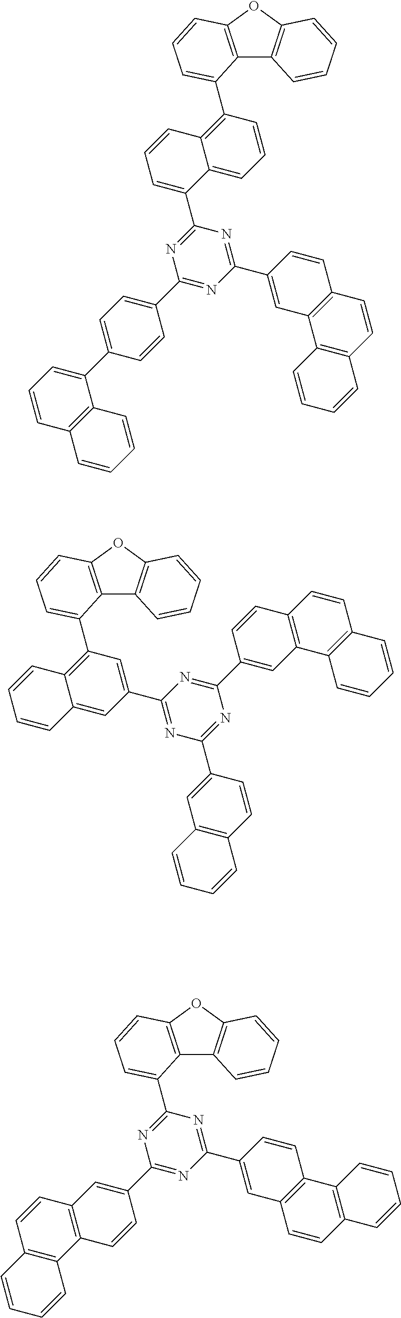 [SMILES: c1ccc2c(-c3ccc(-c4nc(-c5ccc6ccc7ccccc7c6c5)nc(-c5cccc6c(-c7cccc8oc9ccccc9c78)cccc56)n4)cc3)cccc2c1 ; c1ccc2cc(-c3nc(-c4cc(-c5cccc6oc7ccccc7c56)c5ccccc5c4)nc(-c4ccc5ccc6ccccc6c5c4)n3)ccc2c1 ; c1ccc2c(c1)ccc1cc(-c3nc(-c4ccc5c(ccc6ccccc65)c4)nc(-c4cccc5oc6ccccc6c45)n3)ccc12]

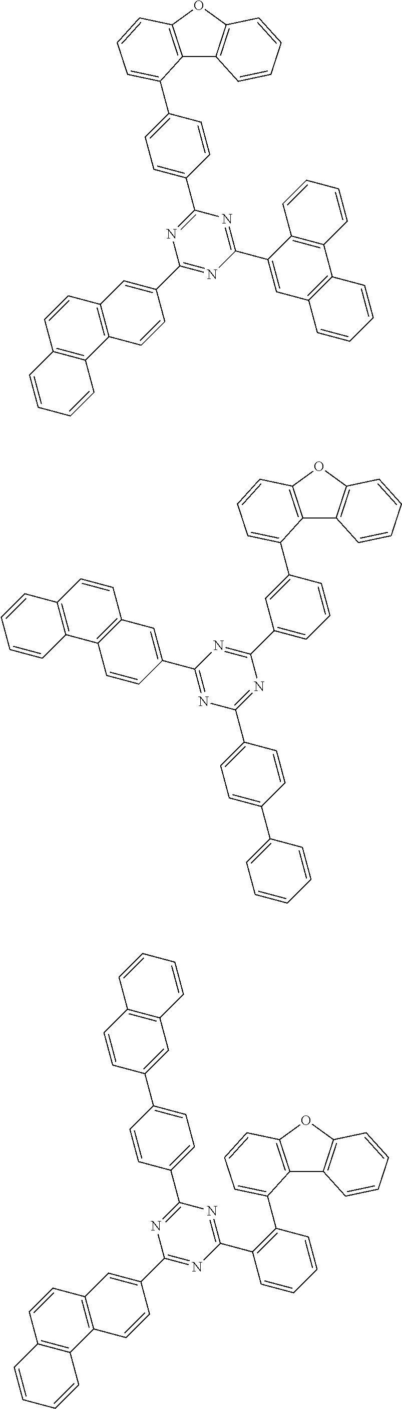 [SMILES: c1ccc2c(c1)ccc1cc(-c3nc(-c4ccc(-c5cccc6oc7ccccc7c56)cc4)nc(-c4cc5ccccc5c5ccccc45)n3)ccc12 ; c1ccc(-c2ccc(-c3nc(-c4cccc(-c5cccc6oc7ccccc7c56)c4)nc(-c4ccc5c(ccc6ccccc65)c4)n3)cc2)cc1 ; c1ccc(-c2cccc3oc4ccccc4c23)c(-c2nc(-c3ccc(-c4ccc5ccccc5c4)cc3)nc(-c3ccc4c(ccc5ccccc54)c3)n2)c1]

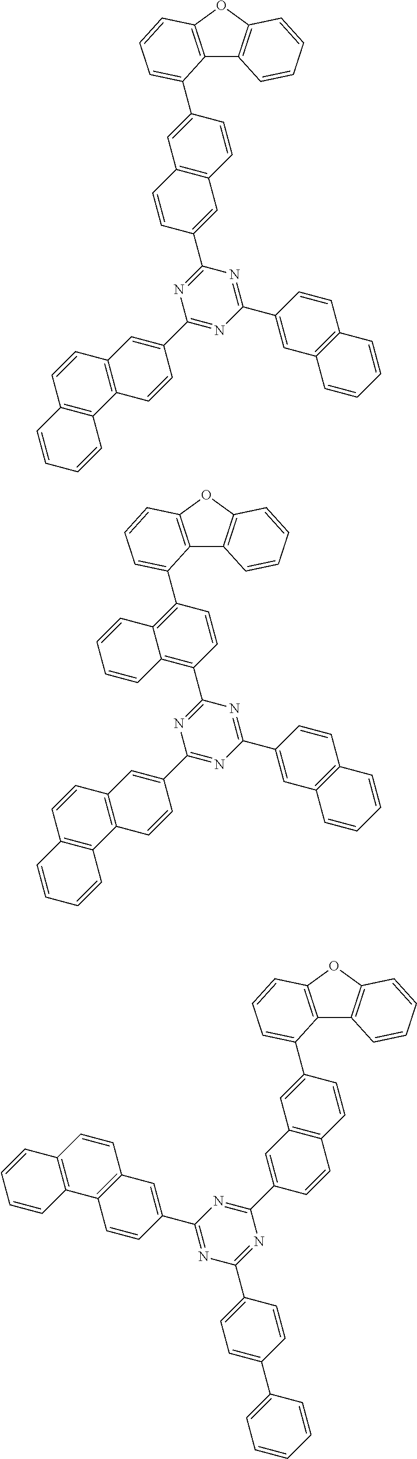 [SMILES: c1ccc2cc(-c3nc(-c4ccc5cc(-c6cccc7oc8ccccc8c67)ccc5c4)nc(-c4ccc5c(ccc6ccccc65)c4)n3)ccc2c1 ; c1ccc2cc(-c3nc(-c4ccc5c(ccc6ccccc65)c4)nc(-c4ccc(-c5cccc6oc7ccccc7c56)c5ccccc45)n3)ccc2c1 ; c1ccc(-c2ccc(-c3nc(-c4ccc5ccc(-c6cccc7oc8ccccc8c67)cc5c4)nc(-c4ccc5c(ccc6ccccc65)c4)n3)cc2)cc1]

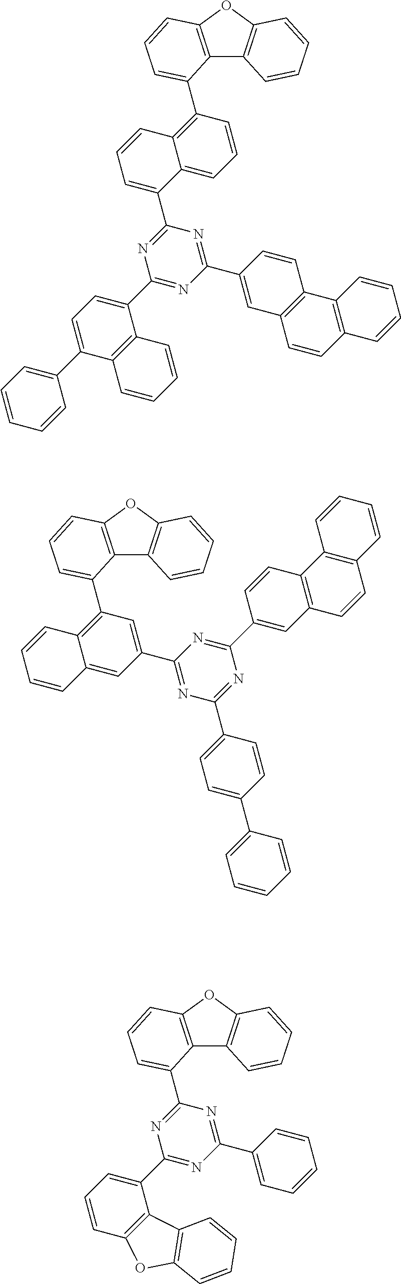 [SMILES: c1ccc(-c2ccc(-c3nc(-c4ccc5c(ccc6ccccc65)c4)nc(-c4cccc5c(-c6cccc7oc8ccccc8c67)cccc45)n3)c3ccccc23)cc1 ; c1ccc(-c2ccc(-c3nc(-c4cc(-c5cccc6oc7ccccc7c56)c5ccccc5c4)nc(-c4ccc5c(ccc6ccccc65)c4)n3)cc2)cc1 ; c1ccc(-c2nc(-c3cccc4oc5ccccc5c34)nc(-c3cccc4oc5ccccc5c34)n2)cc1]

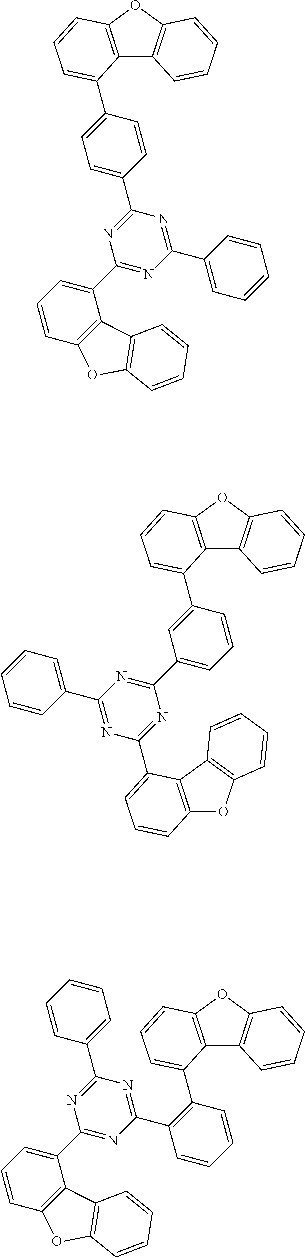 [SMILES: c1ccc(-c2nc(-c3ccc(-c4cccc5oc6ccccc6c45)cc3)nc(-c3cccc4oc5ccccc5c34)n2)cc1 ; c1ccc(-c2nc(-c3cccc(-c4cccc5oc6ccccc6c45)c3)nc(-c3cccc4oc5ccccc5c34)n2)cc1 ; c1ccc(-c2nc(-c3ccccc3-c3cccc4oc5ccccc5c34)nc(-c3cccc4oc5ccccc5c34)n2)cc1]

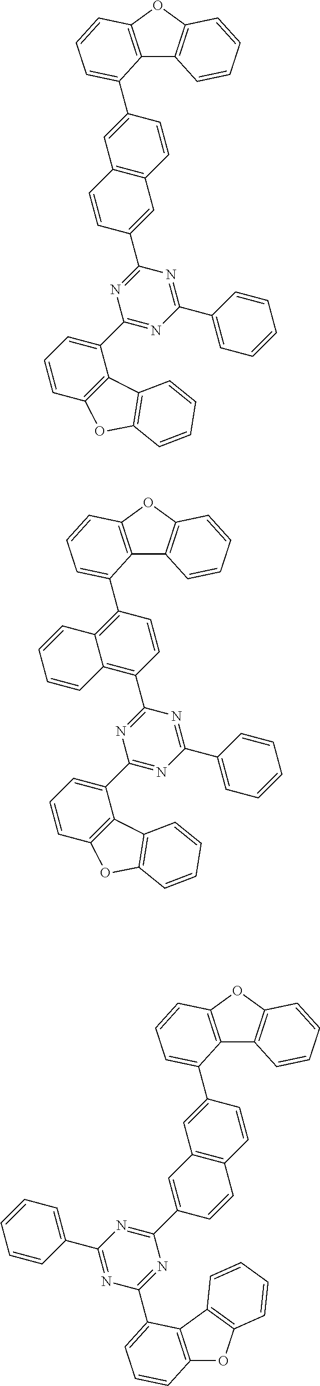 [SMILES: c1ccc(-c2nc(-c3ccc4cc(-c5cccc6oc7ccccc7c56)ccc4c3)nc(-c3cccc4oc5ccccc5c34)n2)cc1 ; c1ccc(-c2nc(-c3ccc(-c4cccc5oc6ccccc6c45)c4ccccc34)nc(-c3cccc4oc5ccccc5c34)n2)cc1 ; c1ccc(-c2nc(-c3ccc4ccc(-c5cccc6oc7ccccc7c56)cc4c3)nc(-c3cccc4oc5ccccc5c34)n2)cc1]

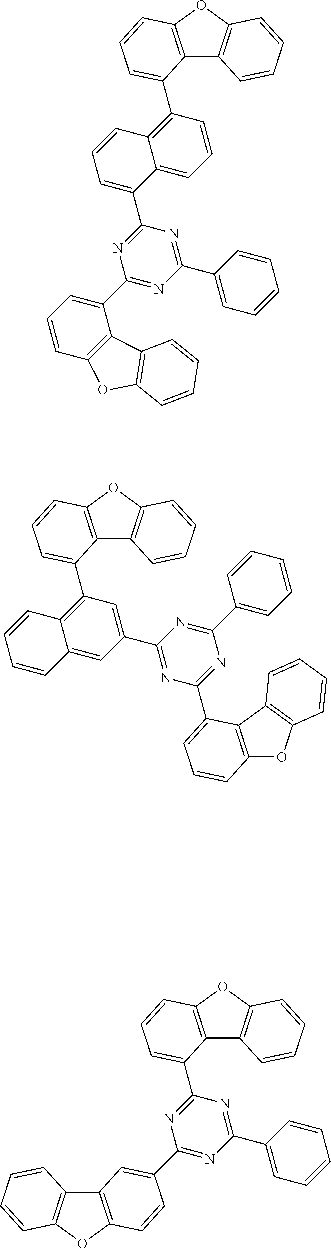 [SMILES: c1ccc(-c2nc(-c3cccc4c(-c5cccc6oc7ccccc7c56)cccc34)nc(-c3cccc4oc5ccccc5c34)n2)cc1 ; c1ccc(-c2nc(-c3cc(-c4cccc5oc6ccccc6c45)c4ccccc4c3)nc(-c3cccc4oc5ccccc5c34)n2)cc1 ; c1ccc(-c2nc(-c3ccc4oc5ccccc5c4c3)nc(-c3cccc4oc5ccccc5c34)n2)cc1]

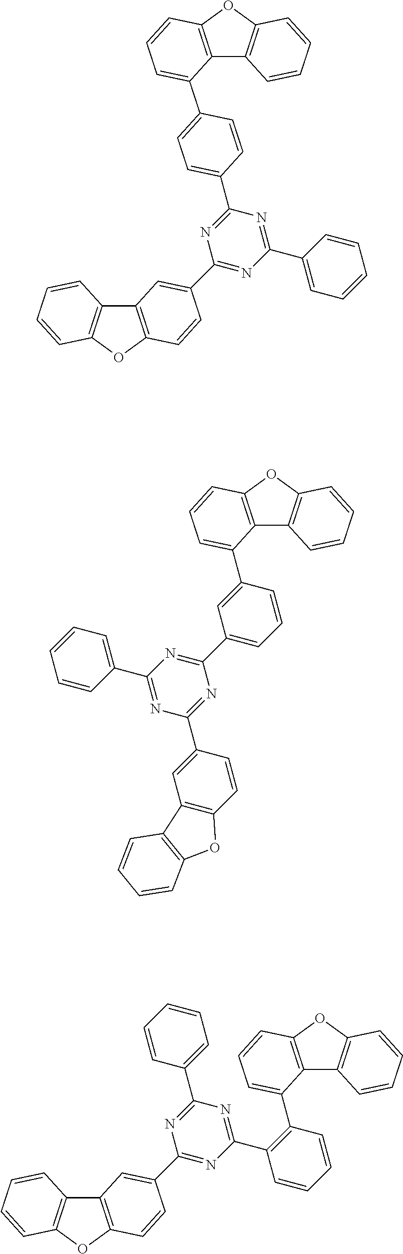 [SMILES: c1ccc(-c2nc(-c3ccc(-c4cccc5oc6ccccc6c45)cc3)nc(-c3ccc4oc5ccccc5c4c3)n2)cc1 ; c1ccc(-c2nc(-c3cccc(-c4cccc5oc6ccccc6c45)c3)nc(-c3ccc4oc5ccccc5c4c3)n2)cc1 ; c1ccc(-c2nc(-c3ccc4oc5ccccc5c4c3)nc(-c3ccccc3-c3cccc4oc5ccccc5c34)n2)cc1]

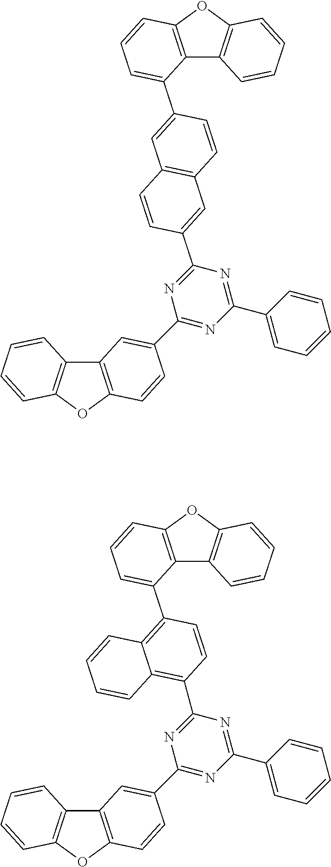 [SMILES: c1ccc(-c2nc(-c3ccc4cc(-c5cccc6oc7ccccc7c56)ccc4c3)nc(-c3ccc4oc5ccccc5c4c3)n2)cc1 ; c1ccc(-c2nc(-c3ccc4oc5ccccc5c4c3)nc(-c3ccc(-c4cccc5oc6ccccc6c45)c4ccccc34)n2)cc1]

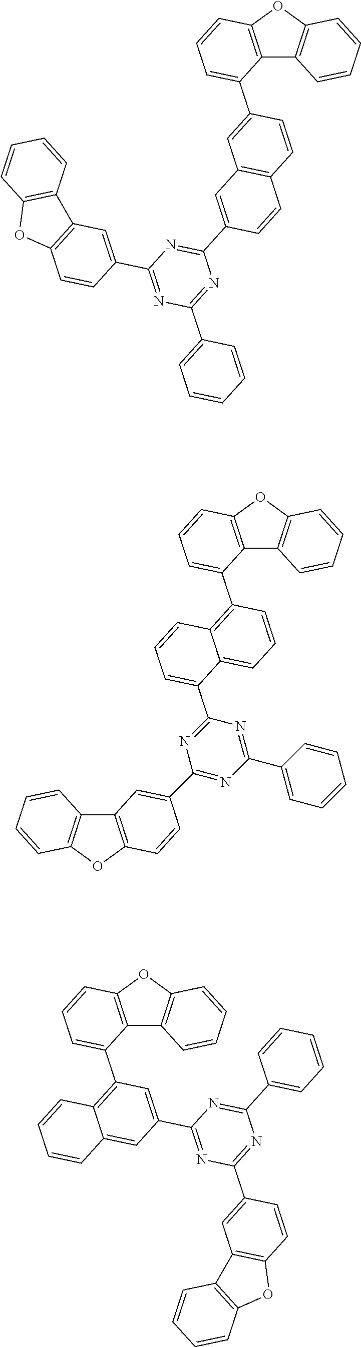 [SMILES: c1ccc(-c2nc(-c3ccc4ccc(-c5cccc6oc7ccccc7c56)cc4c3)nc(-c3ccc4oc5ccccc5c4c3)n2)cc1 ; c1ccc(-c2nc(-c3ccc4oc5ccccc5c4c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4oc5ccccc5c4c3)n2)cc1]

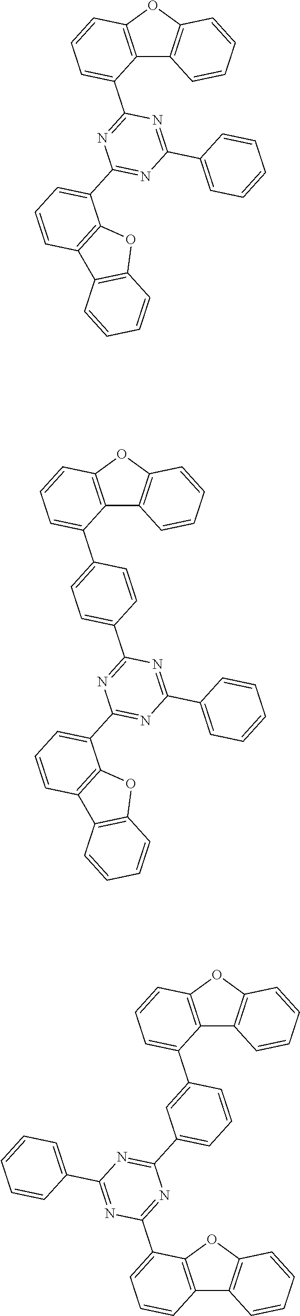 [SMILES: c1ccc(-c2nc(-c3cccc4c3oc3ccccc34)nc(-c3cccc4oc5ccccc5c34)n2)cc1 ; c1ccc(-c2nc(-c3ccc(-c4cccc5oc6ccccc6c45)cc3)nc(-c3cccc4c3oc3ccccc34)n2)cc1 ; c1ccc(-c2nc(-c3cccc(-c4cccc5oc6ccccc6c45)c3)nc(-c3cccc4c3oc3ccccc34)n2)cc1]

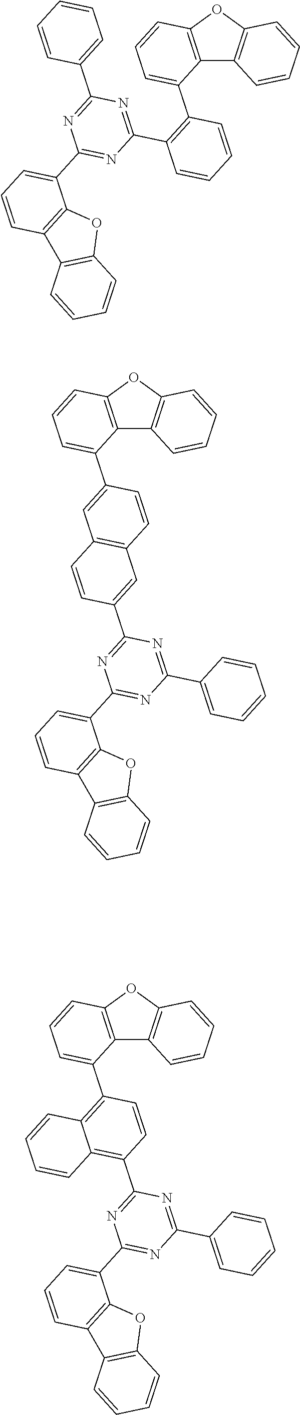 [SMILES: c1ccc(-c2nc(-c3ccccc3-c3cccc4oc5ccccc5c34)nc(-c3cccc4c3oc3ccccc34)n2)cc1 ; c1ccc(-c2nc(-c3ccc4cc(-c5cccc6oc7ccccc7c56)ccc4c3)nc(-c3cccc4c3oc3ccccc34)n2)cc1 ; c1ccc(-c2nc(-c3ccc(-c4cccc5oc6ccccc6c45)c4ccccc34)nc(-c3cccc4c3oc3ccccc34)n2)cc1]

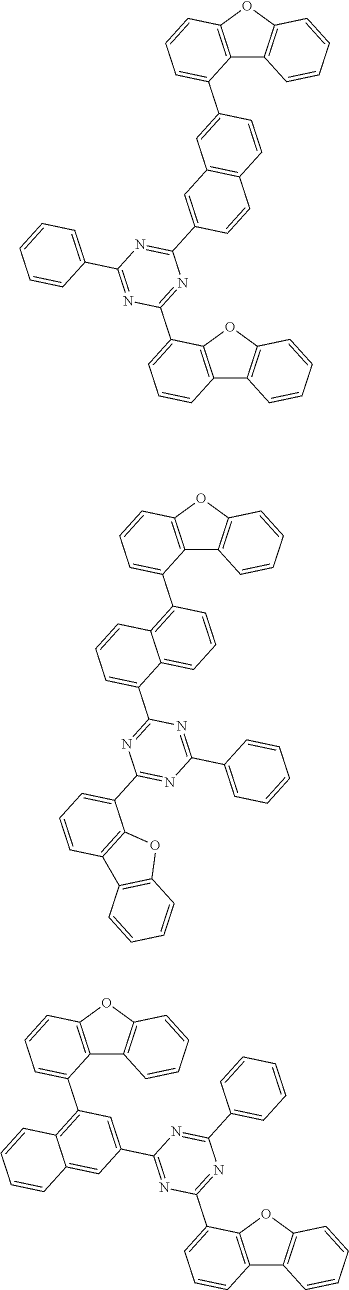 [SMILES: c1ccc(-c2nc(-c3ccc4ccc(-c5cccc6oc7ccccc7c56)cc4c3)nc(-c3cccc4c3oc3ccccc34)n2)cc1 ; c1ccc(-c2nc(-c3cccc4c(-c5cccc6oc7ccccc7c56)cccc34)nc(-c3cccc4c3oc3ccccc34)n2)cc1 ; c1ccc(-c2nc(-c3cc(-c4cccc5oc6ccccc6c45)c4ccccc4c3)nc(-c3cccc4c3oc3ccccc34)n2)cc1]

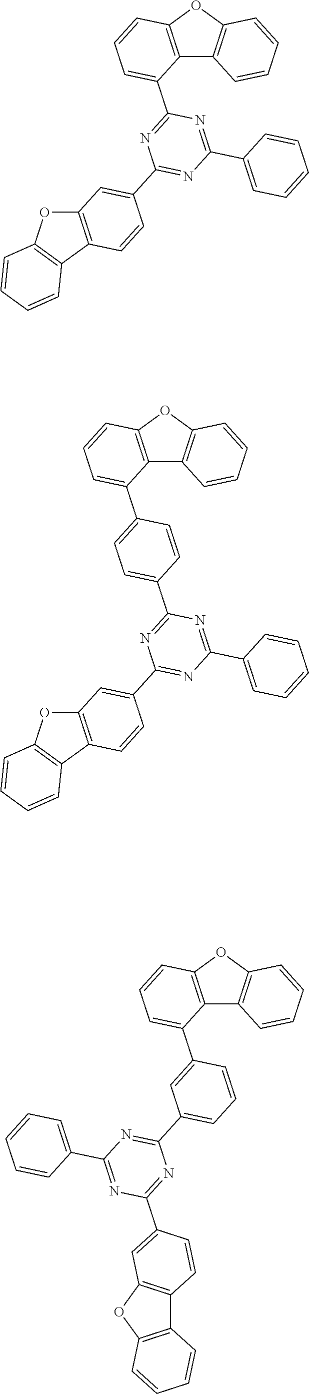 [SMILES: c1ccc(-c2nc(-c3ccc4c(c3)oc3ccccc34)nc(-c3cccc4oc5ccccc5c34)n2)cc1 ; c1ccc(-c2nc(-c3ccc(-c4cccc5oc6ccccc6c45)cc3)nc(-c3ccc4c(c3)oc3ccccc34)n2)cc1 ; c1ccc(-c2nc(-c3cccc(-c4cccc5oc6ccccc6c45)c3)nc(-c3ccc4c(c3)oc3ccccc34)n2)cc1]

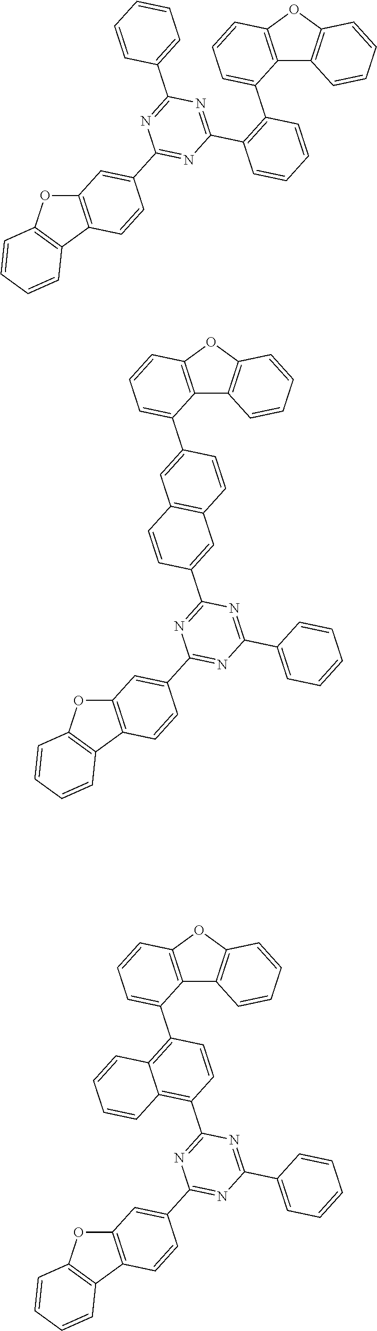 [SMILES: c1ccc(-c2nc(-c3ccc4c(c3)oc3ccccc34)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(c3)oc3ccccc34)n2)cc1 ; c1ccc(-c2nc(-c3ccc4c(c3)oc3ccccc34)nc(-c3ccc(-c4cccc5oc6ccccc6c45)c4ccccc34)n2)cc1]

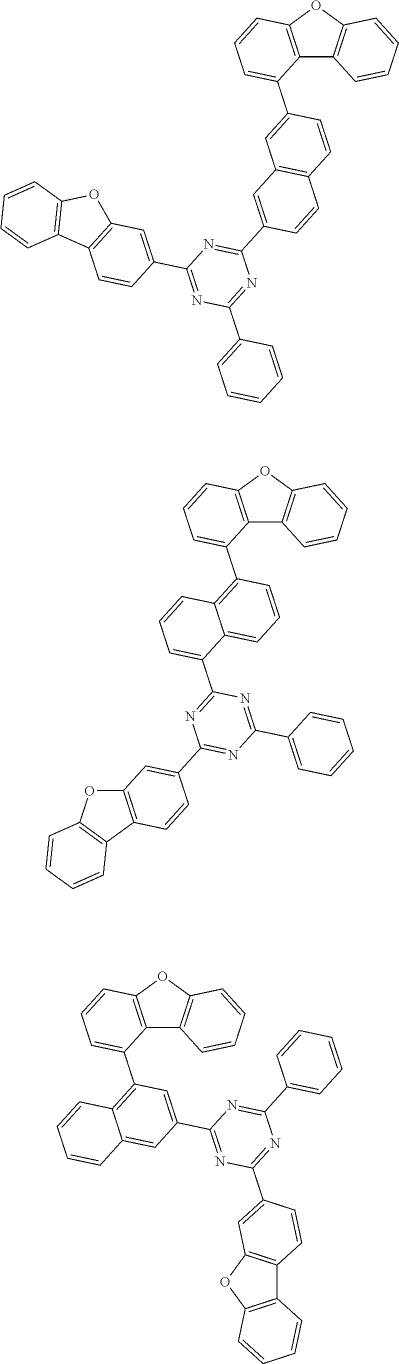 [SMILES: c1ccc(-c2nc(-c3ccc4ccc(-c5cccc6oc7ccccc7c56)cc4c3)nc(-c3ccc4c(c3)oc3ccccc34)n2)cc1 ; c1ccc(-c2nc(-c3ccc4c(c3)oc3ccccc34)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4c(c3)oc3ccccc34)n2)cc1]

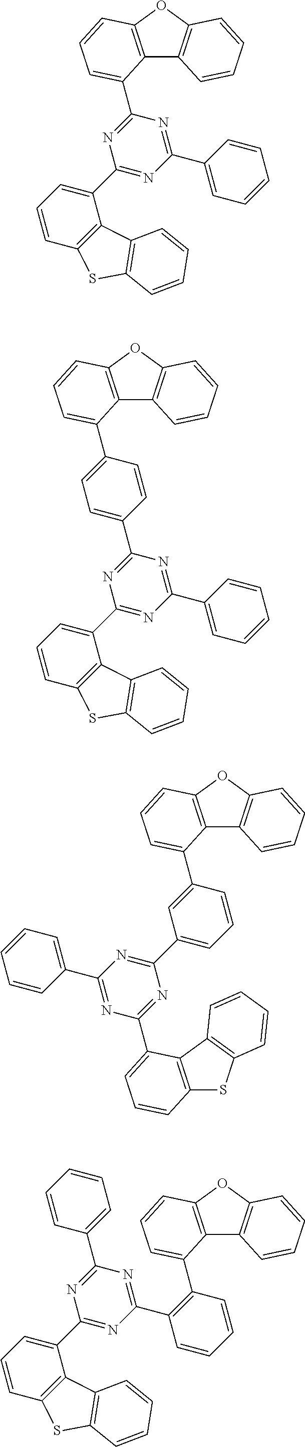 [SMILES: c1ccc(-c2nc(-c3cccc4oc5ccccc5c34)nc(-c3cccc4sc5ccccc5c34)n2)cc1 ; c1ccc(-c2nc(-c3ccc(-c4cccc5oc6ccccc6c45)cc3)nc(-c3cccc4sc5ccccc5c34)n2)cc1 ; c1ccc(-c2nc(-c3cccc(-c4cccc5oc6ccccc6c45)c3)nc(-c3cccc4sc5ccccc5c34)n2)cc1 ; c1ccc(-c2nc(-c3ccccc3-c3cccc4oc5ccccc5c34)nc(-c3cccc4sc5ccccc5c34)n2)cc1]

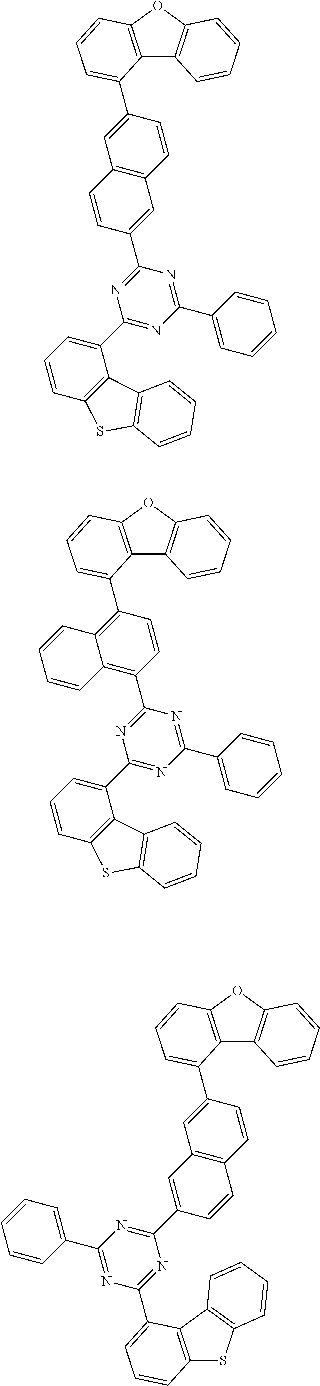 [SMILES: c1ccc(-c2nc(-c3ccc4cc(-c5cccc6oc7ccccc7c56)ccc4c3)nc(-c3cccc4sc5ccccc5c34)n2)cc1 ; c1ccc(-c2nc(-c3ccc(-c4cccc5oc6ccccc6c45)c4ccccc34)nc(-c3cccc4sc5ccccc5c34)n2)cc1 ; c1ccc(-c2nc(-c3ccc4ccc(-c5cccc6oc7ccccc7c56)cc4c3)nc(-c3cccc4sc5ccccc5c34)n2)cc1]

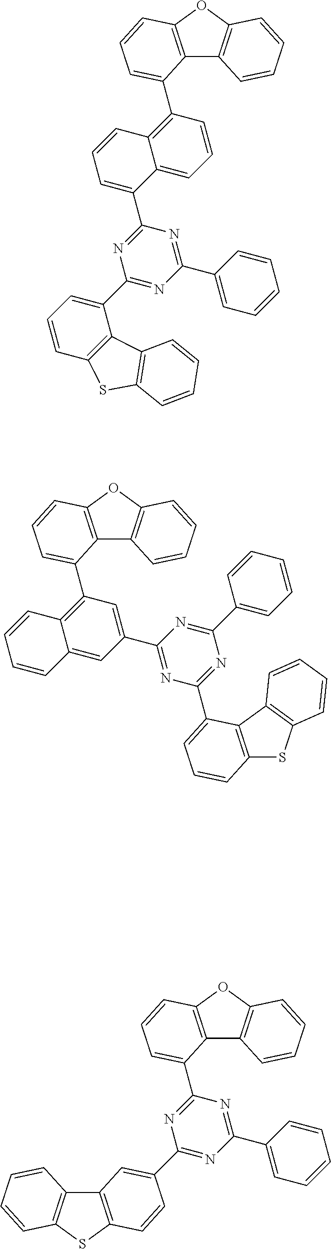 [SMILES: c1ccc(-c2nc(-c3cccc4c(-c5cccc6oc7ccccc7c56)cccc34)nc(-c3cccc4sc5ccccc5c34)n2)cc1 ; c1ccc(-c2nc(-c3cc(-c4cccc5oc6ccccc6c45)c4ccccc4c3)nc(-c3cccc4sc5ccccc5c34)n2)cc1 ; c1ccc(-c2nc(-c3ccc4sc5ccccc5c4c3)nc(-c3cccc4oc5ccccc5c34)n2)cc1]

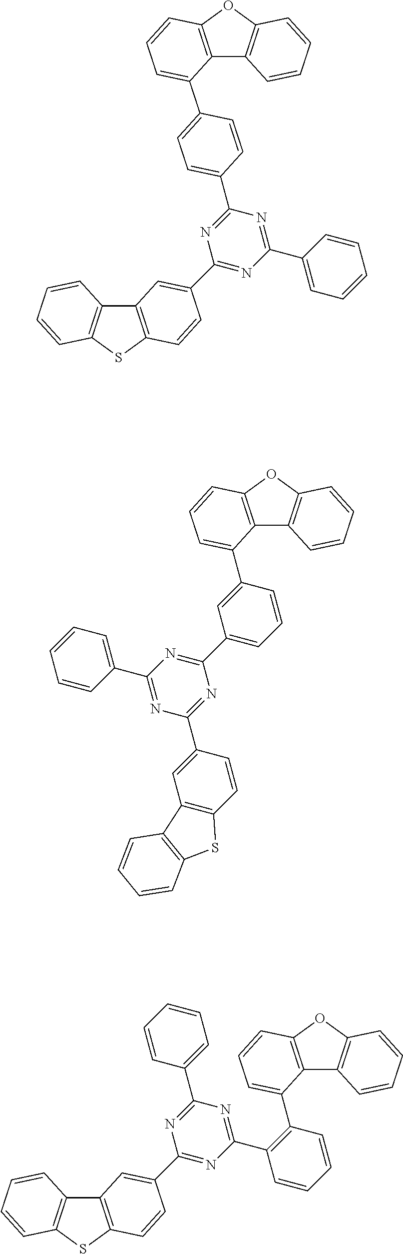 [SMILES: c1ccc(-c2nc(-c3ccc(-c4cccc5oc6ccccc6c45)cc3)nc(-c3ccc4sc5ccccc5c4c3)n2)cc1 ; c1ccc(-c2nc(-c3cccc(-c4cccc5oc6ccccc6c45)c3)nc(-c3ccc4sc5ccccc5c4c3)n2)cc1 ; c1ccc(-c2nc(-c3ccc4sc5ccccc5c4c3)nc(-c3ccccc3-c3cccc4oc5ccccc5c34)n2)cc1]

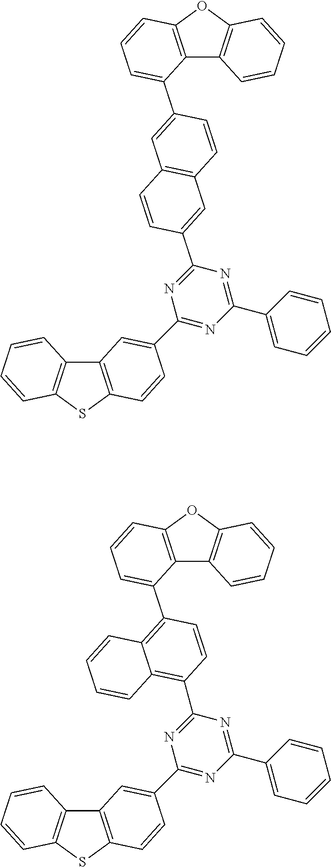 [SMILES: c1ccc(-c2nc(-c3ccc4cc(-c5cccc6oc7ccccc7c56)ccc4c3)nc(-c3ccc4sc5ccccc5c4c3)n2)cc1 ; c1ccc(-c2nc(-c3ccc4sc5ccccc5c4c3)nc(-c3ccc(-c4cccc5oc6ccccc6c45)c4ccccc34)n2)cc1]

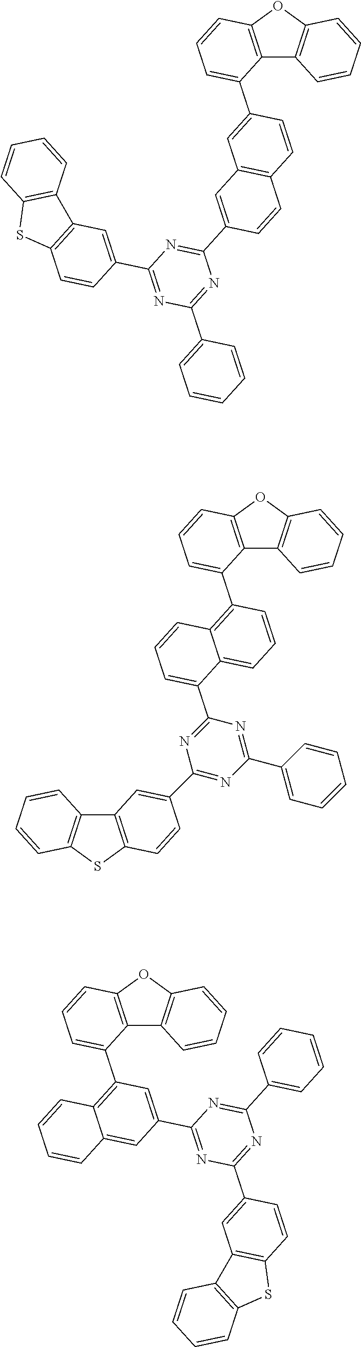 [SMILES: c1ccc(-c2nc(-c3ccc4ccc(-c5cccc6oc7ccccc7c56)cc4c3)nc(-c3ccc4sc5ccccc5c4c3)n2)cc1 ; c1ccc(-c2nc(-c3ccc4sc5ccccc5c4c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4sc5ccccc5c4c3)n2)cc1]

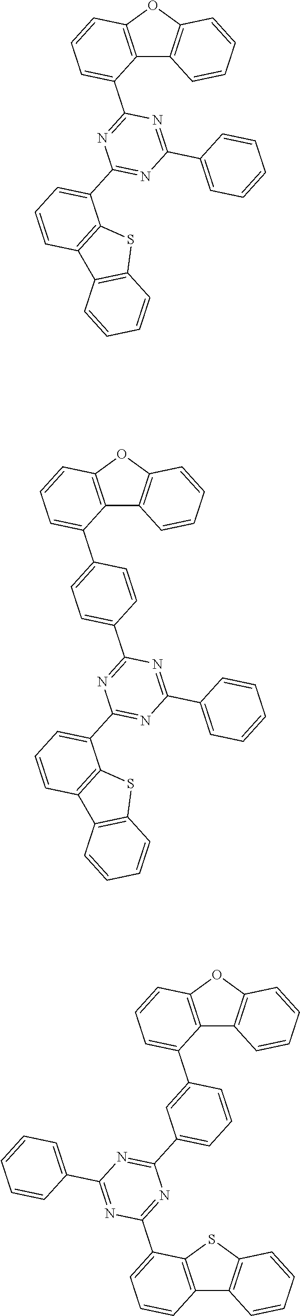 [SMILES: c1ccc(-c2nc(-c3cccc4c3sc3ccccc34)nc(-c3cccc4oc5ccccc5c34)n2)cc1 ; c1ccc(-c2nc(-c3ccc(-c4cccc5oc6ccccc6c45)cc3)nc(-c3cccc4c3sc3ccccc34)n2)cc1 ; c1ccc(-c2nc(-c3cccc(-c4cccc5oc6ccccc6c45)c3)nc(-c3cccc4c3sc3ccccc34)n2)cc1]

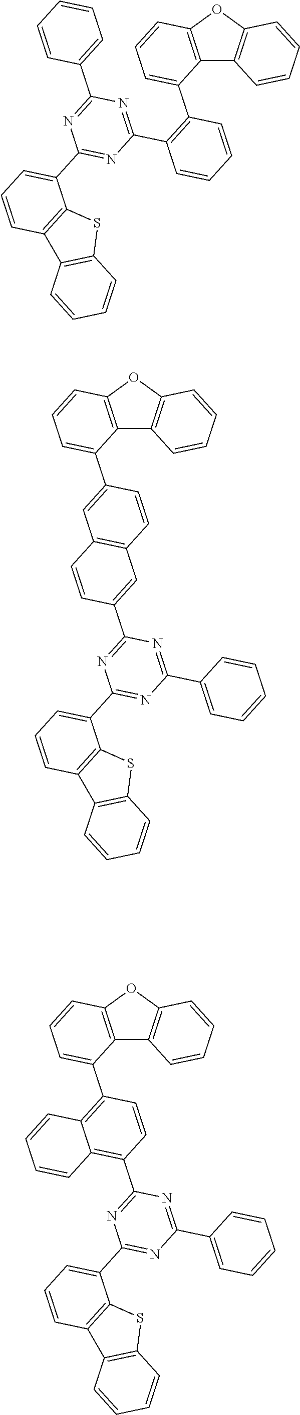 [SMILES: c1ccc(-c2nc(-c3ccccc3-c3cccc4oc5ccccc5c34)nc(-c3cccc4c3sc3ccccc34)n2)cc1 ; c1ccc(-c2nc(-c3ccc4cc(-c5cccc6oc7ccccc7c56)ccc4c3)nc(-c3cccc4c3sc3ccccc34)n2)cc1 ; c1ccc(-c2nc(-c3ccc(-c4cccc5oc6ccccc6c45)c4ccccc34)nc(-c3cccc4c3sc3ccccc34)n2)cc1]

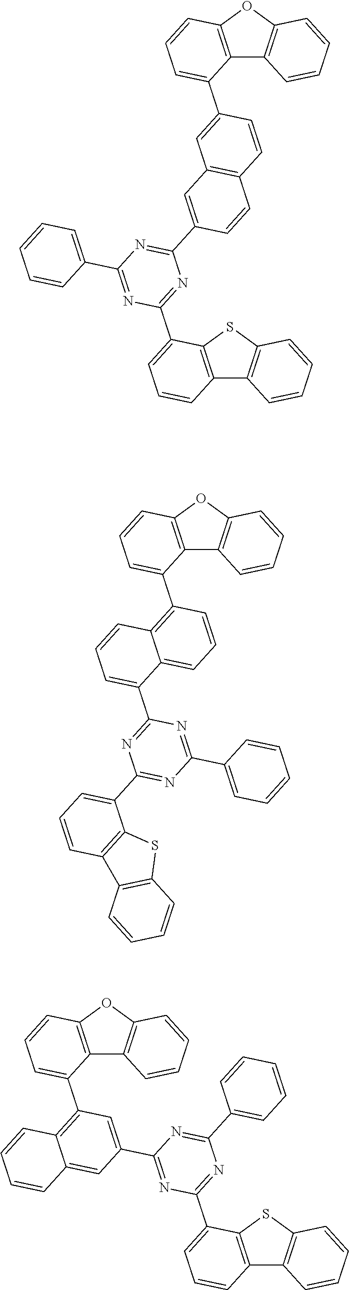 [SMILES: c1ccc(-c2nc(-c3ccc4ccc(-c5cccc6oc7ccccc7c56)cc4c3)nc(-c3cccc4c3sc3ccccc34)n2)cc1 ; c1ccc(-c2nc(-c3cccc4c(-c5cccc6oc7ccccc7c56)cccc34)nc(-c3cccc4c3sc3ccccc34)n2)cc1 ; c1ccc(-c2nc(-c3cc(-c4cccc5oc6ccccc6c45)c4ccccc4c3)nc(-c3cccc4c3sc3ccccc34)n2)cc1]

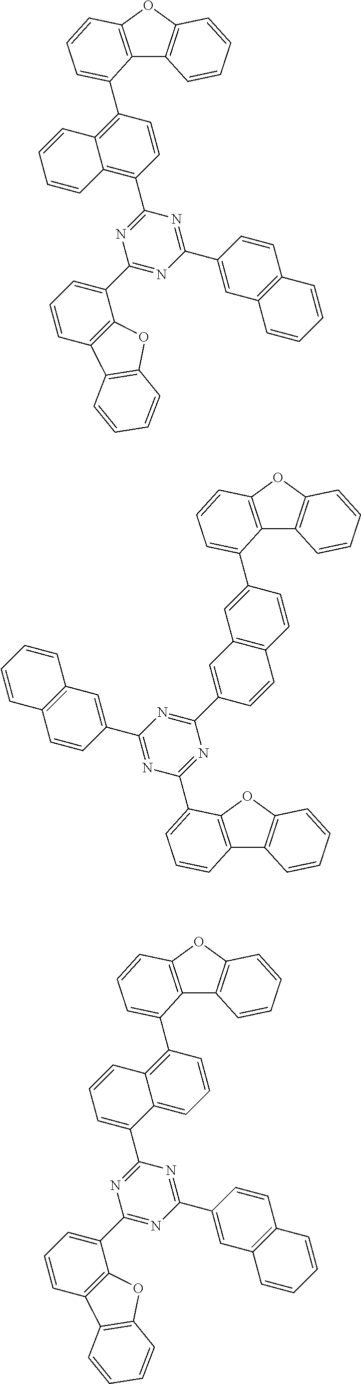 [SMILES: c1ccc2cc(-c3nc(-c4ccc(-c5cccc6oc7ccccc7c56)c5ccccc45)nc(-c4cccc5c4oc4ccccc45)n3)ccc2c1 ; c1ccc2cc(-c3nc(-c4ccc5ccc(-c6cccc7oc8ccccc8c67)cc5c4)nc(-c4cccc5c4oc4ccccc45)n3)ccc2c1 ; c1ccc2cc(-c3nc(-c4cccc5c(-c6cccc7oc8ccccc8c67)cccc45)nc(-c4cccc5c4oc4ccccc45)n3)ccc2c1]

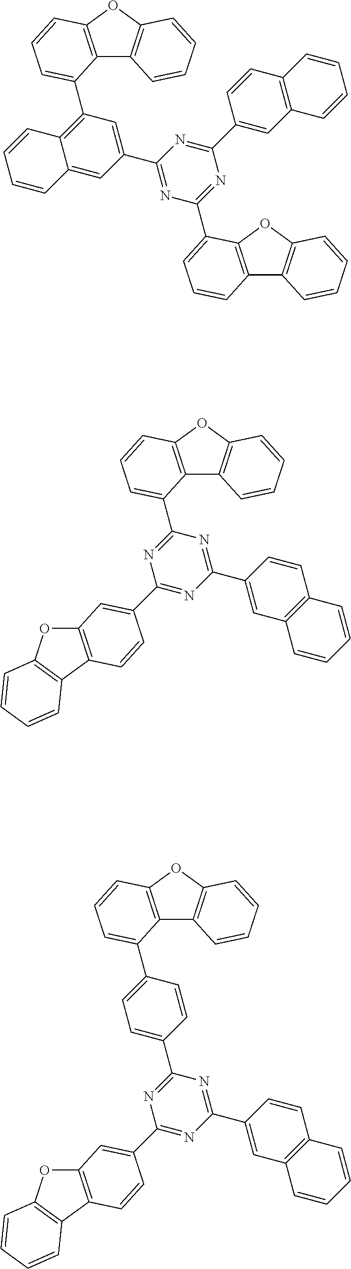 [SMILES: c1ccc2cc(-c3nc(-c4cc(-c5cccc6oc7ccccc7c56)c5ccccc5c4)nc(-c4cccc5c4oc4ccccc45)n3)ccc2c1 ; c1ccc2cc(-c3nc(-c4ccc5c(c4)oc4ccccc45)nc(-c4cccc5oc6ccccc6c45)n3)ccc2c1 ; c1ccc2cc(-c3nc(-c4ccc(-c5cccc6oc7ccccc7c56)cc4)nc(-c4ccc5c(c4)oc4ccccc45)n3)ccc2c1]

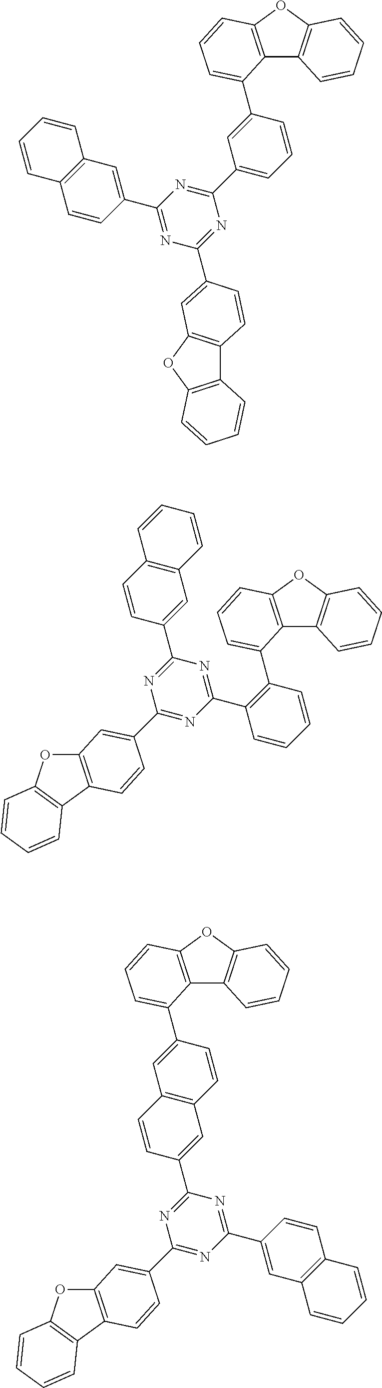 [SMILES: c1cc(-c2nc(-c3ccc4ccccc4c3)nc(-c3ccc4c(c3)oc3ccccc34)n2)cc(-c2cccc3oc4ccccc4c23)c1 ; c1ccc(-c2cccc3oc4ccccc4c23)c(-c2nc(-c3ccc4ccccc4c3)nc(-c3ccc4c(c3)oc3ccccc34)n2)c1 ; c1ccc2cc(-c3nc(-c4ccc5cc(-c6cccc7oc8ccccc8c67)ccc5c4)nc(-c4ccc5c(c4)oc4ccccc45)n3)ccc2c1]

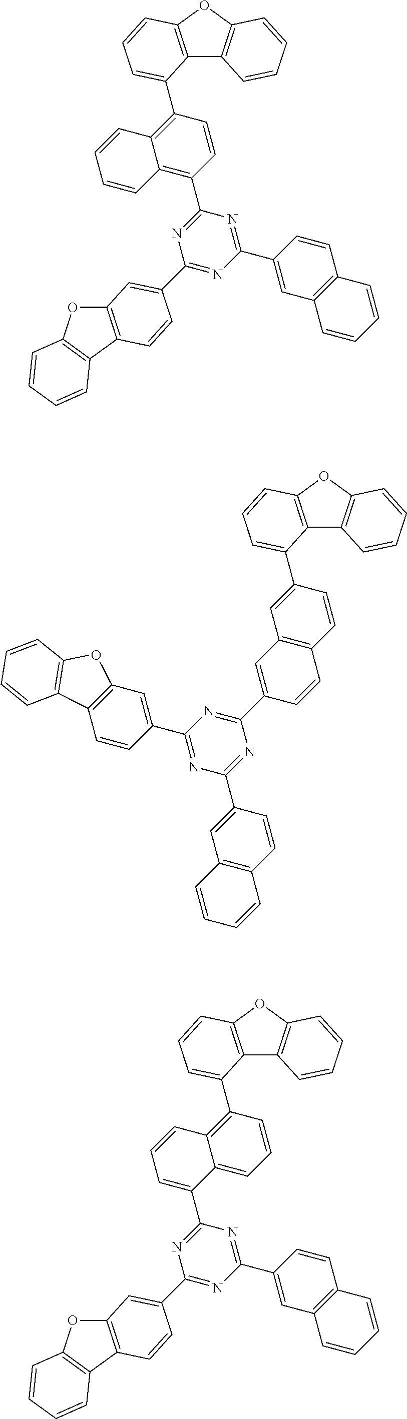 [SMILES: c1ccc2cc(-c3nc(-c4ccc5c(c4)oc4ccccc45)nc(-c4ccc(-c5cccc6oc7ccccc7c56)c5ccccc45)n3)ccc2c1 ; c1ccc2cc(-c3nc(-c4ccc5ccc(-c6cccc7oc8ccccc8c67)cc5c4)nc(-c4ccc5c(c4)oc4ccccc45)n3)ccc2c1 ; c1ccc2cc(-c3nc(-c4ccc5c(c4)oc4ccccc45)nc(-c4cccc5c(-c6cccc7oc8ccccc8c67)cccc45)n3)ccc2c1]

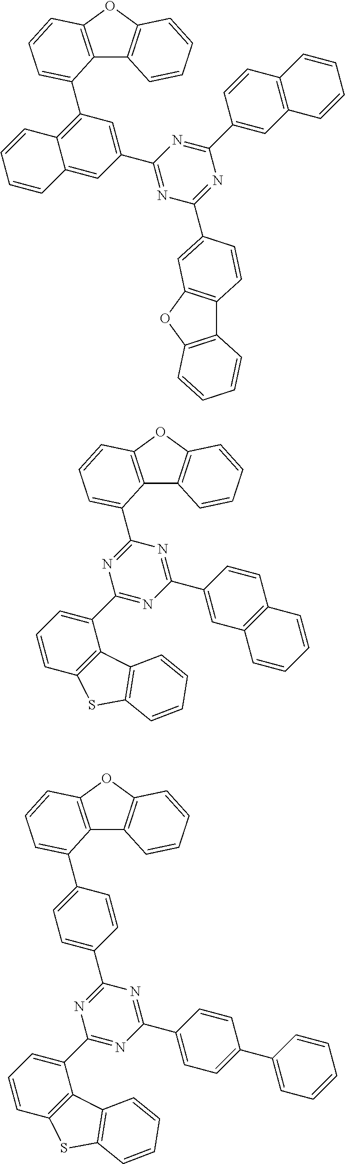 [SMILES: c1ccc2cc(-c3nc(-c4cc(-c5cccc6oc7ccccc7c56)c5ccccc5c4)nc(-c4ccc5c(c4)oc4ccccc45)n3)ccc2c1 ; c1ccc2cc(-c3nc(-c4cccc5oc6ccccc6c45)nc(-c4cccc5sc6ccccc6c45)n3)ccc2c1 ; c1ccc(-c2ccc(-c3nc(-c4ccc(-c5cccc6oc7ccccc7c56)cc4)nc(-c4cccc5sc6ccccc6c45)n3)cc2)cc1]

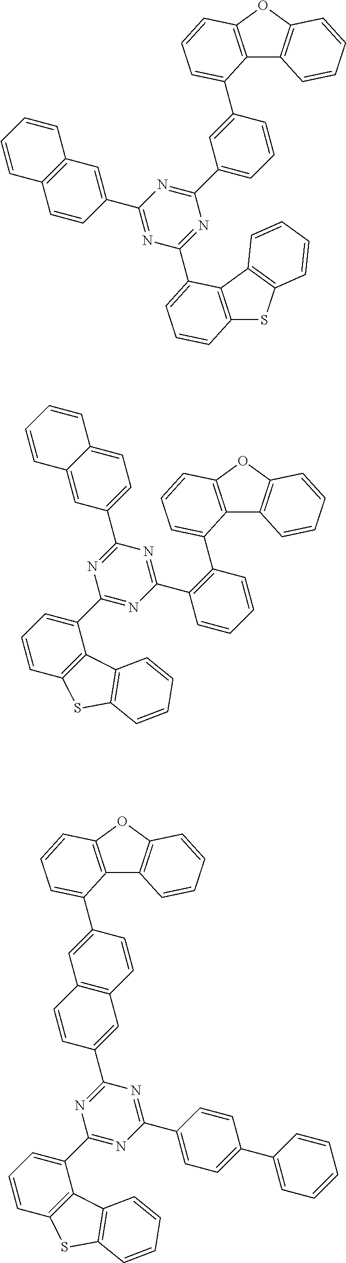 [SMILES: c1cc(-c2nc(-c3ccc4ccccc4c3)nc(-c3cccc4sc5ccccc5c34)n2)cc(-c2cccc3oc4ccccc4c23)c1 ; c1ccc(-c2cccc3oc4ccccc4c23)c(-c2nc(-c3ccc4ccccc4c3)nc(-c3cccc4sc5ccccc5c34)n2)c1 ; c1ccc(-c2ccc(-c3nc(-c4ccc5cc(-c6cccc7oc8ccccc8c67)ccc5c4)nc(-c4cccc5sc6ccccc6c45)n3)cc2)cc1]

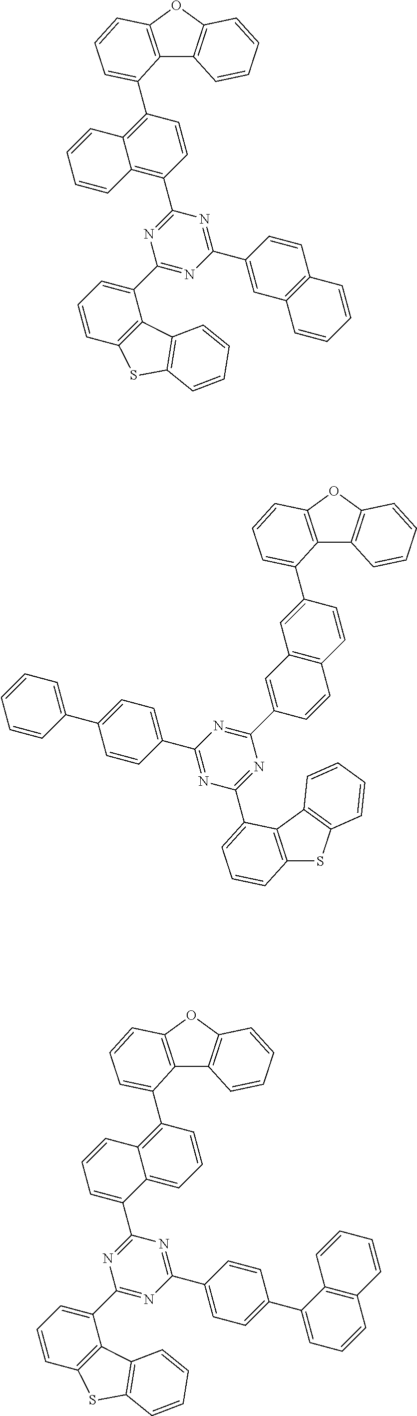 [SMILES: c1ccc2cc(-c3nc(-c4ccc(-c5cccc6oc7ccccc7c56)c5ccccc45)nc(-c4cccc5sc6ccccc6c45)n3)ccc2c1 ; c1ccc(-c2ccc(-c3nc(-c4ccc5ccc(-c6cccc7oc8ccccc8c67)cc5c4)nc(-c4cccc5sc6ccccc6c45)n3)cc2)cc1 ; c1ccc2c(-c3ccc(-c4nc(-c5cccc6c(-c7cccc8oc9ccccc9c78)cccc56)nc(-c5cccc6sc7ccccc7c56)n4)cc3)cccc2c1]

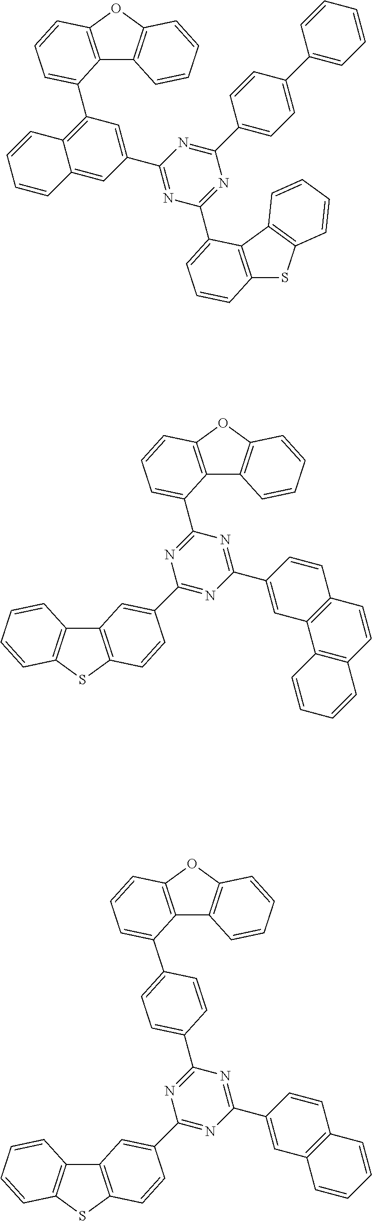 [SMILES: c1ccc(-c2ccc(-c3nc(-c4cc(-c5cccc6oc7ccccc7c56)c5ccccc5c4)nc(-c4cccc5sc6ccccc6c45)n3)cc2)cc1 ; c1ccc2c(c1)ccc1ccc(-c3nc(-c4ccc5sc6ccccc6c5c4)nc(-c4cccc5oc6ccccc6c45)n3)cc12 ; c1ccc2cc(-c3nc(-c4ccc(-c5cccc6oc7ccccc7c56)cc4)nc(-c4ccc5sc6ccccc6c5c4)n3)ccc2c1]

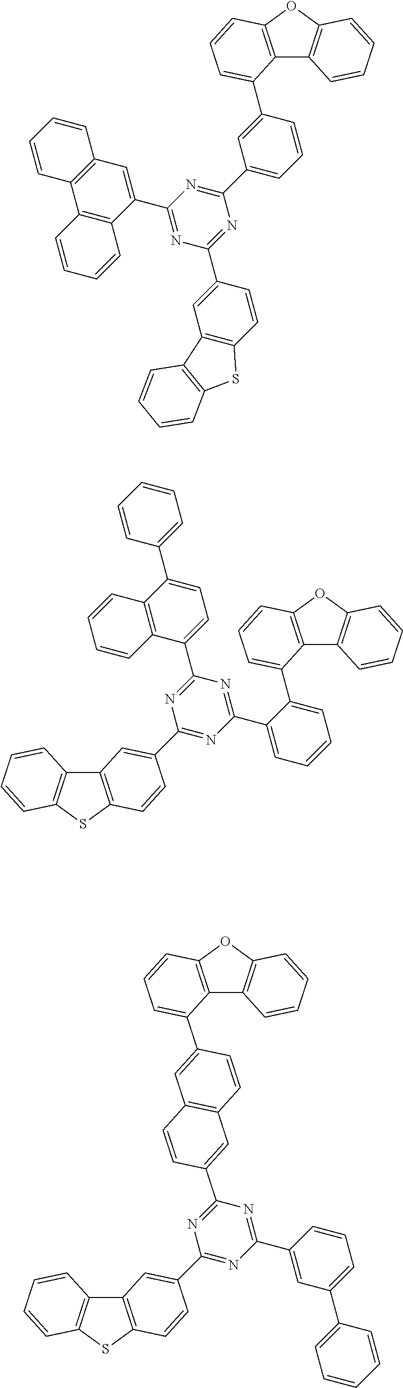 [SMILES: c1cc(-c2nc(-c3ccc4sc5ccccc5c4c3)nc(-c3cc4ccccc4c4ccccc34)n2)cc(-c2cccc3oc4ccccc4c23)c1 ; c1ccc(-c2ccc(-c3nc(-c4ccc5sc6ccccc6c5c4)nc(-c4ccccc4-c4cccc5oc6ccccc6c45)n3)c3ccccc23)cc1 ; c1ccc(-c2cccc(-c3nc(-c4ccc5cc(-c6cccc7oc8ccccc8c67)ccc5c4)nc(-c4ccc5sc6ccccc6c5c4)n3)c2)cc1]

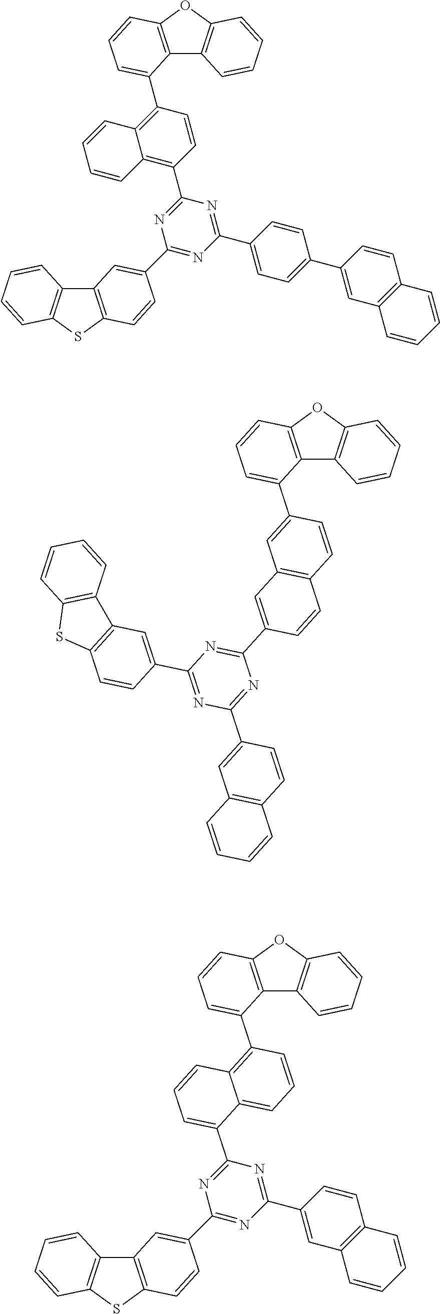 [SMILES: c1ccc2cc(-c3ccc(-c4nc(-c5ccc6sc7ccccc7c6c5)nc(-c5ccc(-c6cccc7oc8ccccc8c67)c6ccccc56)n4)cc3)ccc2c1 ; c1ccc2cc(-c3nc(-c4ccc5ccc(-c6cccc7oc8ccccc8c67)cc5c4)nc(-c4ccc5sc6ccccc6c5c4)n3)ccc2c1 ; c1ccc2cc(-c3nc(-c4ccc5sc6ccccc6c5c4)nc(-c4cccc5c(-c6cccc7oc8ccccc8c67)cccc45)n3)ccc2c1]

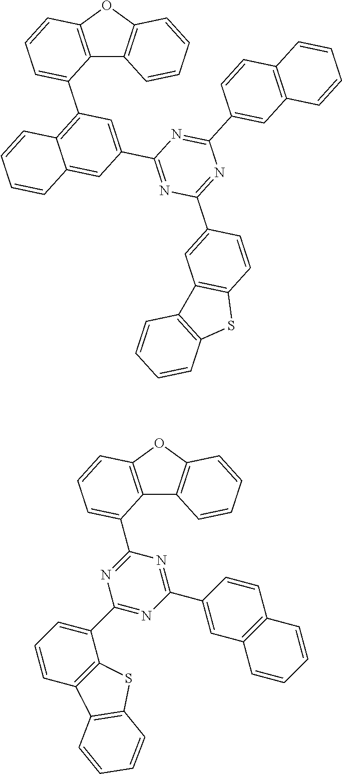 [SMILES: c1ccc2cc(-c3nc(-c4cc(-c5cccc6oc7ccccc7c56)c5ccccc5c4)nc(-c4ccc5sc6ccccc6c5c4)n3)ccc2c1 ; c1ccc2cc(-c3nc(-c4cccc5c4sc4ccccc45)nc(-c4cccc5oc6ccccc6c45)n3)ccc2c1]

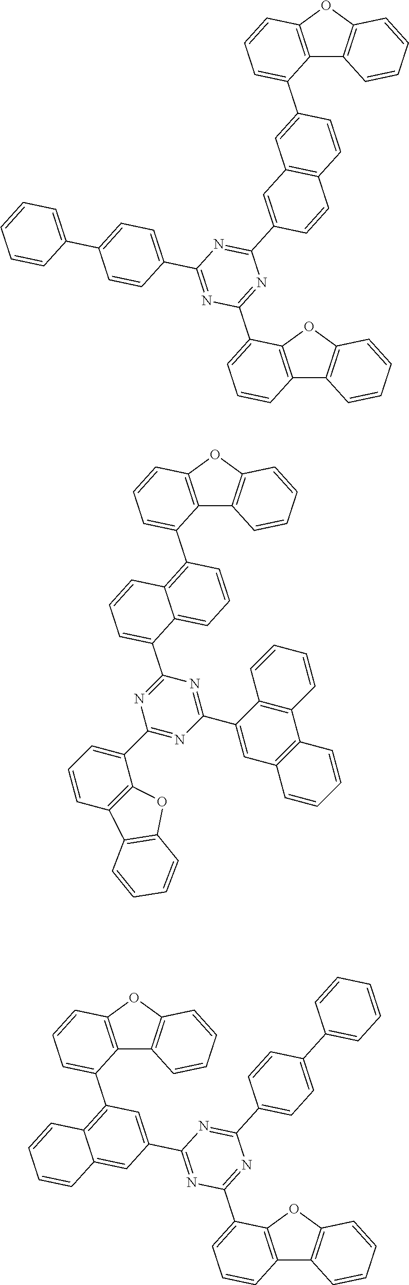 [SMILES: c1ccc(-c2ccc(-c3nc(-c4ccc5ccc(-c6cccc7oc8ccccc8c67)cc5c4)nc(-c4cccc5c4oc4ccccc45)n3)cc2)cc1 ; c1ccc2c(c1)cc(-c1nc(-c3cccc4c(-c5cccc6oc7ccccc7c56)cccc34)nc(-c3cccc4c3oc3ccccc34)n1)c1ccccc12 ; c1ccc(-c2ccc(-c3nc(-c4cc(-c5cccc6oc7ccccc7c56)c5ccccc5c4)nc(-c4cccc5c4oc4ccccc45)n3)cc2)cc1]

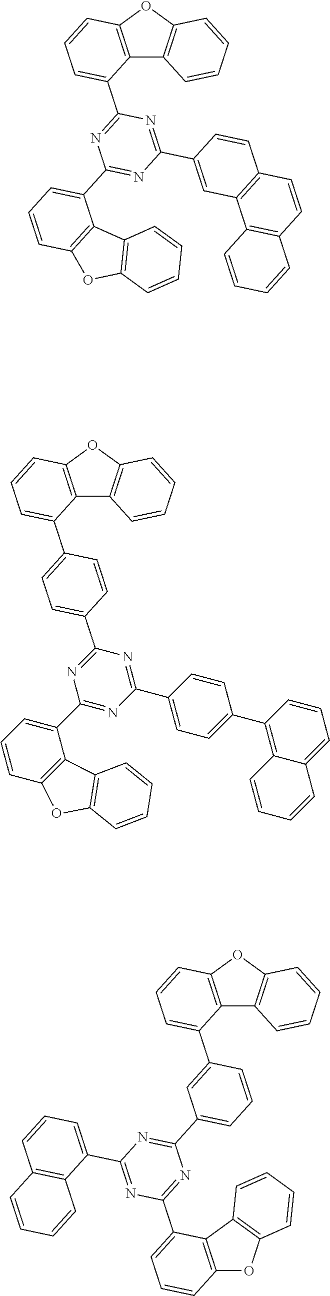 [SMILES: c1ccc2c(c1)ccc1ccc(-c3nc(-c4cccc5oc6ccccc6c45)nc(-c4cccc5oc6ccccc6c45)n3)cc12 ; c1ccc2c(-c3ccc(-c4nc(-c5ccc(-c6cccc7oc8ccccc8c67)cc5)nc(-c5cccc6oc7ccccc7c56)n4)cc3)cccc2c1 ; c1cc(-c2nc(-c3cccc4ccccc34)nc(-c3cccc4oc5ccccc5c34)n2)cc(-c2cccc3oc4ccccc4c23)c1]

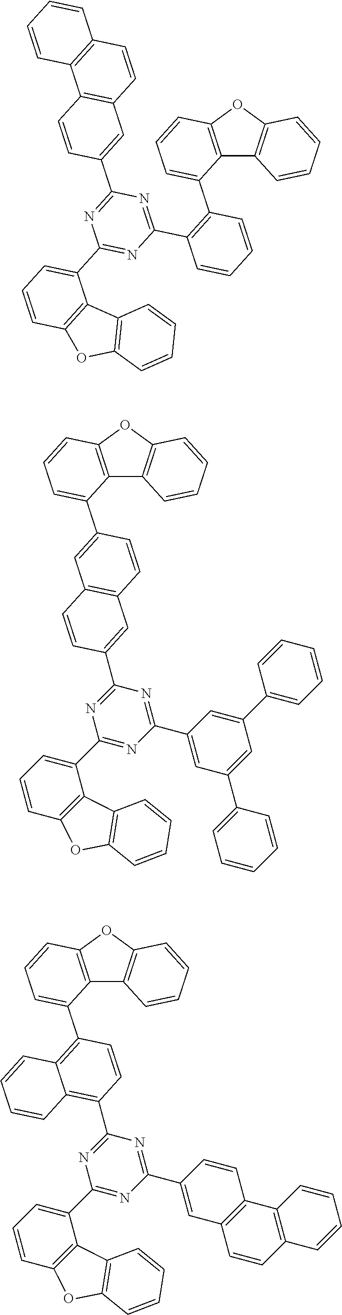 [SMILES: c1ccc(-c2cccc3oc4ccccc4c23)c(-c2nc(-c3ccc4c(ccc5ccccc54)c3)nc(-c3cccc4oc5ccccc5c34)n2)c1 ; c1ccc(-c2cc(-c3ccccc3)cc(-c3nc(-c4ccc5cc(-c6cccc7oc8ccccc8c67)ccc5c4)nc(-c4cccc5oc6ccccc6c45)n3)c2)cc1 ; c1ccc2c(c1)ccc1cc(-c3nc(-c4ccc(-c5cccc6oc7ccccc7c56)c5ccccc45)nc(-c4cccc5oc6ccccc6c45)n3)ccc12]

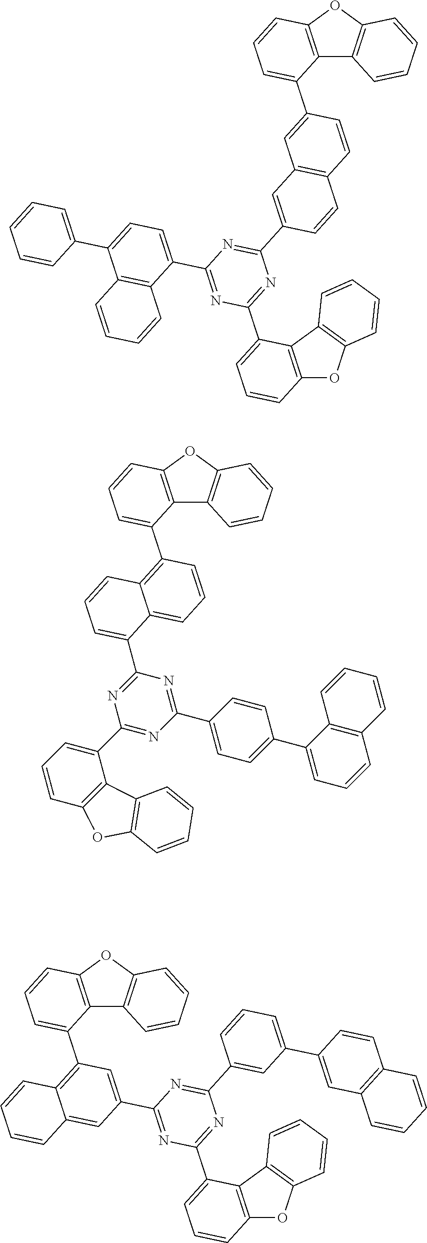 [SMILES: c1ccc(-c2ccc(-c3nc(-c4ccc5ccc(-c6cccc7oc8ccccc8c67)cc5c4)nc(-c4cccc5oc6ccccc6c45)n3)c3ccccc23)cc1 ; c1ccc2c(-c3ccc(-c4nc(-c5cccc6c(-c7cccc8oc9ccccc9c78)cccc56)nc(-c5cccc6oc7ccccc7c56)n4)cc3)cccc2c1 ; c1cc(-c2ccc3ccccc3c2)cc(-c2nc(-c3cc(-c4cccc5oc6ccccc6c45)c4ccccc4c3)nc(-c3cccc4oc5ccccc5c34)n2)c1]

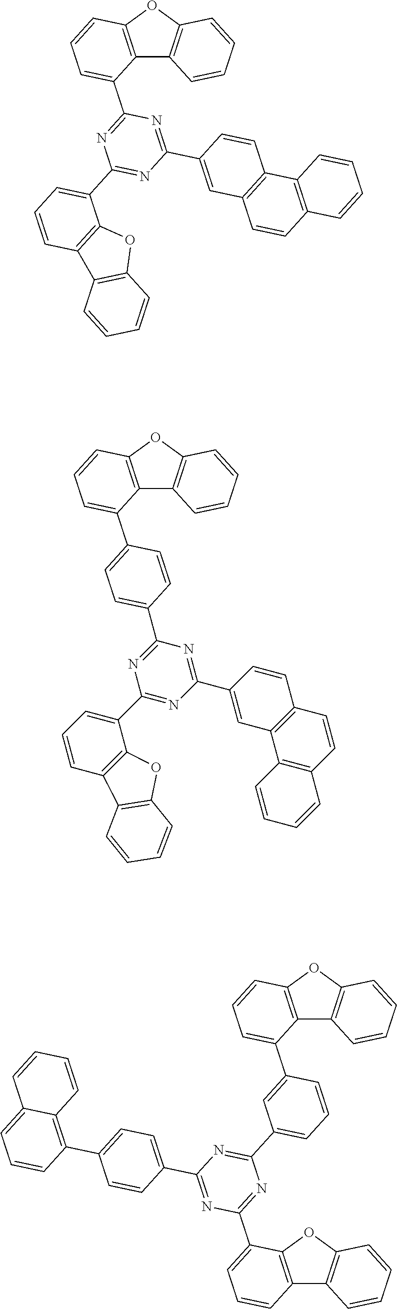 [SMILES: c1ccc2c(c1)ccc1cc(-c3nc(-c4cccc5c4oc4ccccc45)nc(-c4cccc5oc6ccccc6c45)n3)ccc12 ; c1ccc2c(c1)ccc1ccc(-c3nc(-c4ccc(-c5cccc6oc7ccccc7c56)cc4)nc(-c4cccc5c4oc4ccccc45)n3)cc12 ; c1cc(-c2nc(-c3ccc(-c4cccc5ccccc45)cc3)nc(-c3cccc4c3oc3ccccc34)n2)cc(-c2cccc3oc4ccccc4c23)c1]

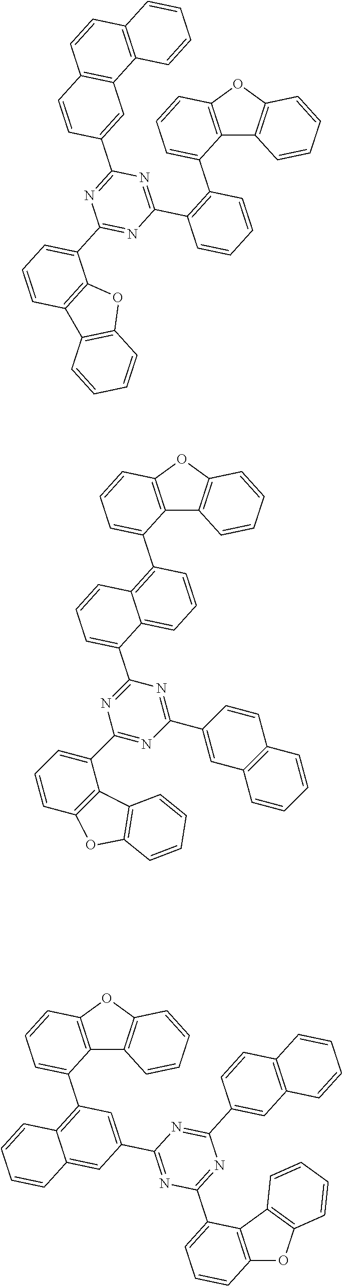 [SMILES: c1ccc(-c2cccc3oc4ccccc4c23)c(-c2nc(-c3ccc4ccc5ccccc5c4c3)nc(-c3cccc4c3oc3ccccc34)n2)c1 ; c1ccc2cc(-c3nc(-c4cccc5c(-c6cccc7oc8ccccc8c67)cccc45)nc(-c4cccc5oc6ccccc6c45)n3)ccc2c1 ; c1ccc2cc(-c3nc(-c4cc(-c5cccc6oc7ccccc7c56)c5ccccc5c4)nc(-c4cccc5oc6ccccc6c45)n3)ccc2c1]

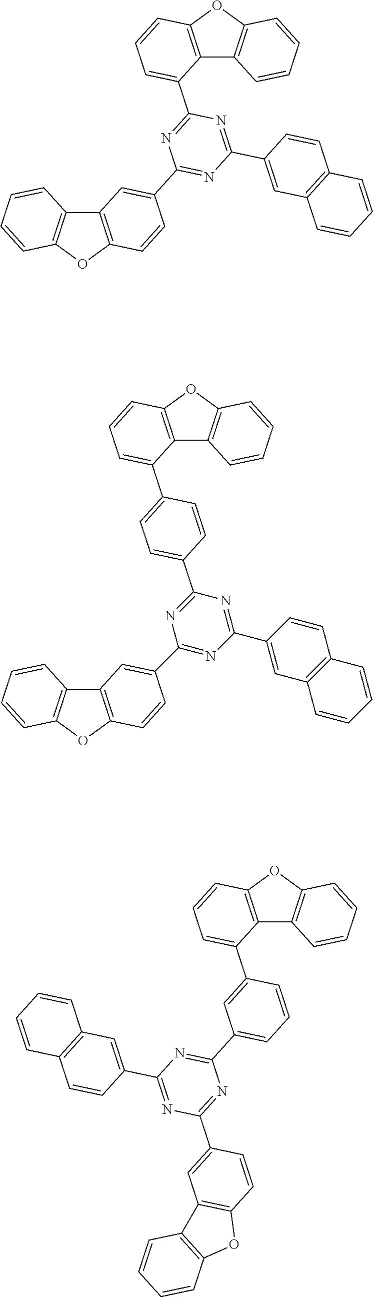 [SMILES: c1ccc2cc(-c3nc(-c4ccc5oc6ccccc6c5c4)nc(-c4cccc5oc6ccccc6c45)n3)ccc2c1 ; c1ccc2cc(-c3nc(-c4ccc(-c5cccc6oc7ccccc7c56)cc4)nc(-c4ccc5oc6ccccc6c5c4)n3)ccc2c1 ; c1cc(-c2nc(-c3ccc4ccccc4c3)nc(-c3ccc4oc5ccccc5c4c3)n2)cc(-c2cccc3oc4ccccc4c23)c1]

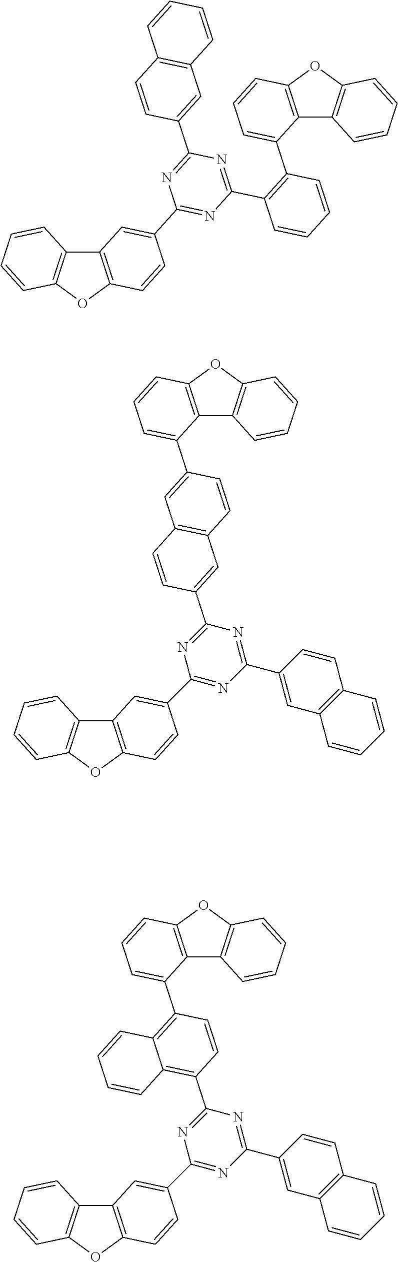 [SMILES: c1ccc(-c2cccc3oc4ccccc4c23)c(-c2nc(-c3ccc4ccccc4c3)nc(-c3ccc4oc5ccccc5c4c3)n2)c1 ; c1ccc2cc(-c3nc(-c4ccc5cc(-c6cccc7oc8ccccc8c67)ccc5c4)nc(-c4ccc5oc6ccccc6c5c4)n3)ccc2c1 ; c1ccc2cc(-c3nc(-c4ccc5oc6ccccc6c5c4)nc(-c4ccc(-c5cccc6oc7ccccc7c56)c5ccccc45)n3)ccc2c1]

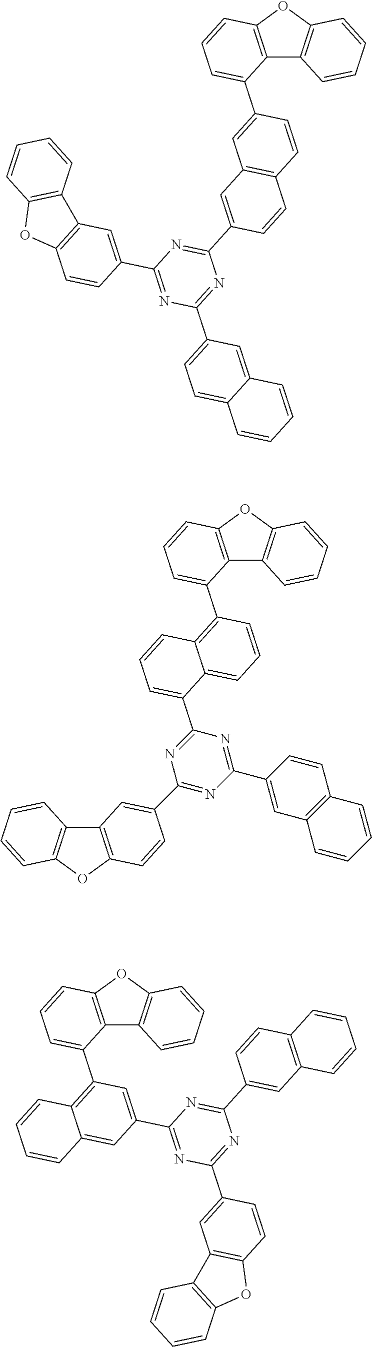 [SMILES: c1ccc2cc(-c3nc(-c4ccc5ccc(-c6cccc7oc8ccccc8c67)cc5c4)nc(-c4ccc5oc6ccccc6c5c4)n3)ccc2c1 ; c1ccc2cc(-c3nc(-c4ccc5oc6ccccc6c5c4)nc(-c4cccc5c(-c6cccc7oc8ccccc8c67)cccc45)n3)ccc2c1 ; c1ccc2cc(-c3nc(-c4cc(-c5cccc6oc7ccccc7c56)c5ccccc5c4)nc(-c4ccc5oc6ccccc6c5c4)n3)ccc2c1]

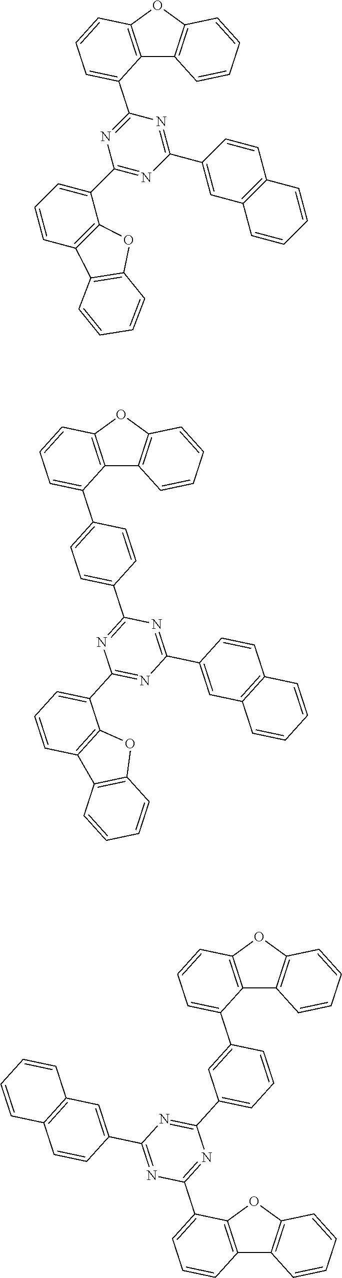 [SMILES: c1ccc2cc(-c3nc(-c4cccc5c4oc4ccccc45)nc(-c4cccc5oc6ccccc6c45)n3)ccc2c1 ; c1ccc2cc(-c3nc(-c4ccc(-c5cccc6oc7ccccc7c56)cc4)nc(-c4cccc5c4oc4ccccc45)n3)ccc2c1 ; c1cc(-c2nc(-c3ccc4ccccc4c3)nc(-c3cccc4c3oc3ccccc34)n2)cc(-c2cccc3oc4ccccc4c23)c1]

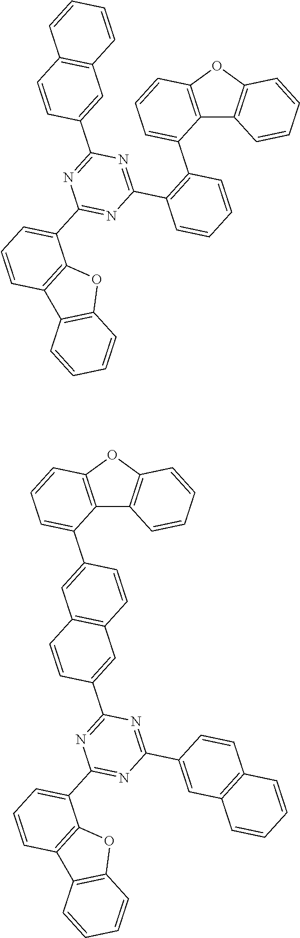 [SMILES: c1ccc(-c2cccc3oc4ccccc4c23)c(-c2nc(-c3ccc4ccccc4c3)nc(-c3cccc4c3oc3ccccc34)n2)c1 ; c1ccc2cc(-c3nc(-c4ccc5cc(-c6cccc7oc8ccccc8c67)ccc5c4)nc(-c4cccc5c4oc4ccccc45)n3)ccc2c1]

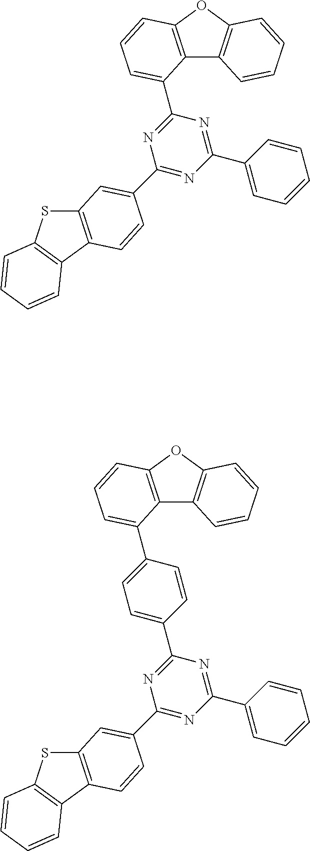 [SMILES: c1ccc(-c2nc(-c3ccc4c(c3)sc3ccccc34)nc(-c3cccc4oc5ccccc5c34)n2)cc1 ; c1ccc(-c2nc(-c3ccc(-c4cccc5oc6ccccc6c45)cc3)nc(-c3ccc4c(c3)sc3ccccc34)n2)cc1]

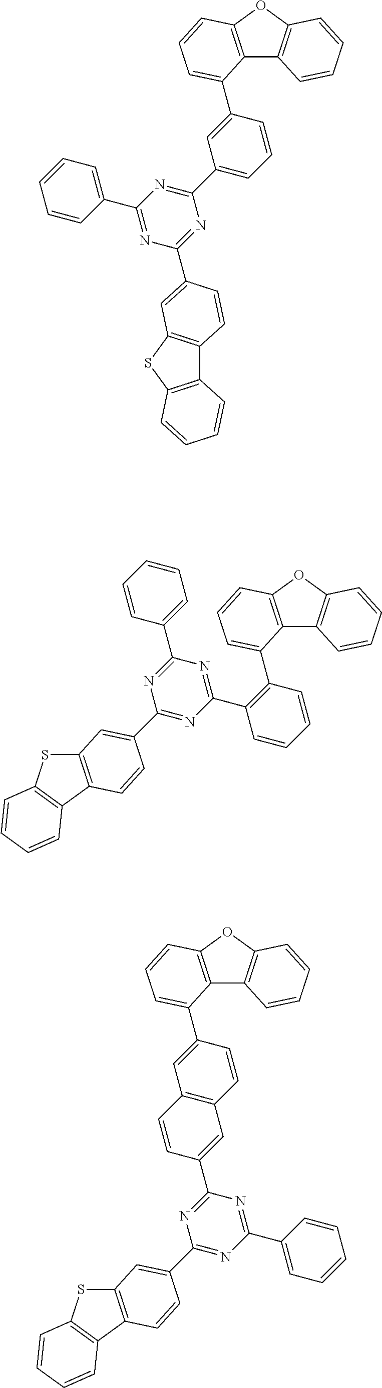 [SMILES: c1ccc(-c2nc(-c3cccc(-c4cccc5oc6ccccc6c45)c3)nc(-c3ccc4c(c3)sc3ccccc34)n2)cc1 ; c1ccc(-c2nc(-c3ccc4c(c3)sc3ccccc34)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(c3)sc3ccccc34)n2)cc1]

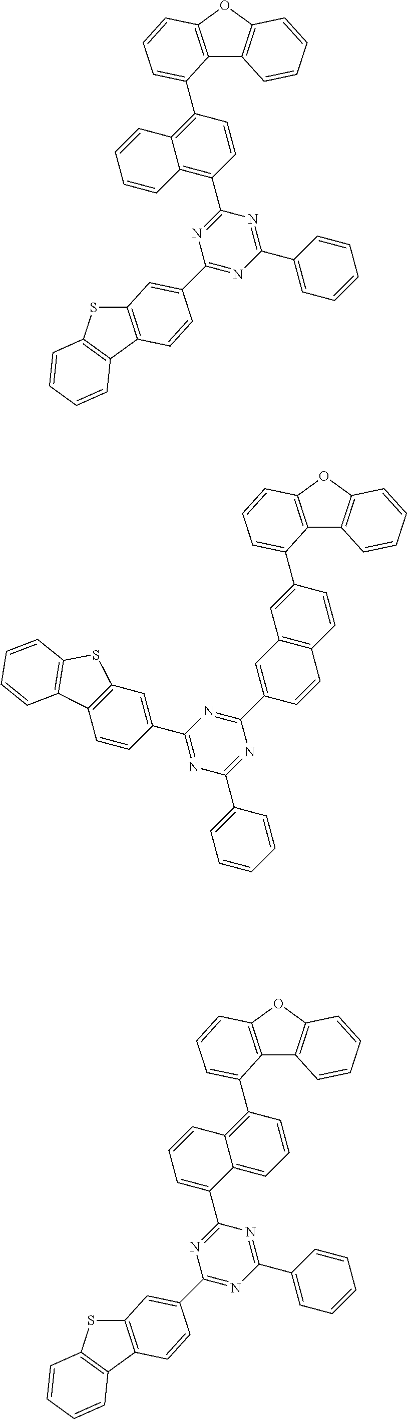 [SMILES: c1ccc(-c2nc(-c3ccc4c(c3)sc3ccccc34)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(c3)sc3ccccc34)n2)cc1 ; c1ccc(-c2nc(-c3ccc4c(c3)sc3ccccc34)nc(-c3cccc4c(-c5cccc6oc7ccccc7c56)cccc34)n2)cc1]

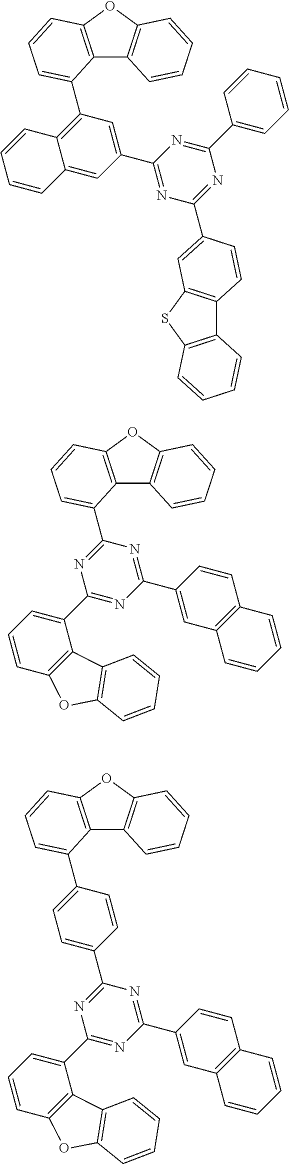

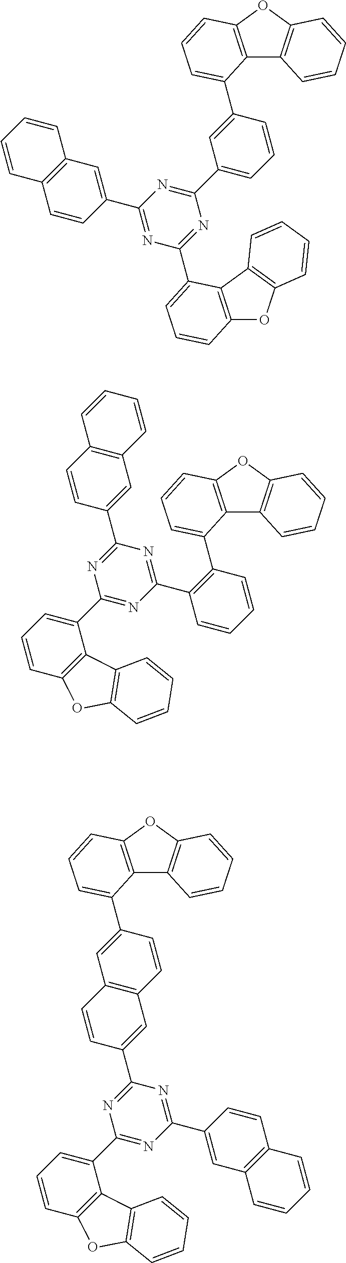 [SMILES: c1cc(-c2nc(-c3ccc4ccccc4c3)nc(-c3cccc4oc5ccccc5c34)n2)cc(-c2cccc3oc4ccccc4c23)c1 ; c1ccc(-c2cccc3oc4ccccc4c23)c(-c2nc(-c3ccc4ccccc4c3)nc(-c3cccc4oc5ccccc5c34)n2)c1 ; c1ccc2cc(-c3nc(-c4ccc5cc(-c6cccc7oc8ccccc8c67)ccc5c4)nc(-c4cccc5oc6ccccc6c45)n3)ccc2c1]

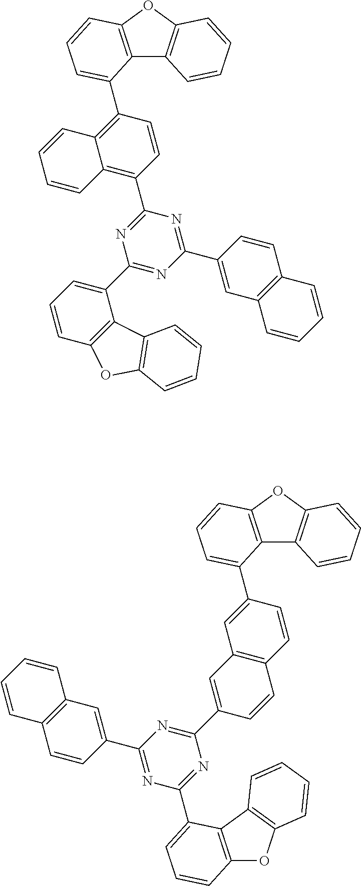 [SMILES: c1ccc2cc(-c3nc(-c4ccc(-c5cccc6oc7ccccc7c56)c5ccccc45)nc(-c4cccc5oc6ccccc6c45)n3)ccc2c1 ; c1ccc2cc(-c3nc(-c4ccc5ccc(-c6cccc7oc8ccccc8c67)cc5c4)nc(-c4cccc5oc6ccccc6c45)n3)ccc2c1]

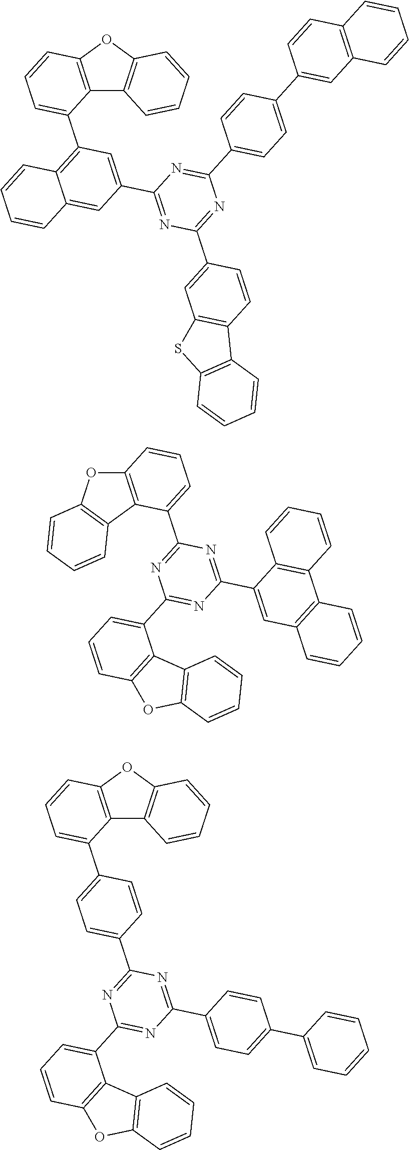 [SMILES: c1ccc2cc(-c3ccc(-c4nc(-c5cc(-c6cccc7oc8ccccc8c67)c6ccccc6c5)nc(-c5ccc6c(c5)sc5ccccc56)n4)cc3)ccc2c1 ; c1ccc2c(c1)cc(-c1nc(-c3cccc4oc5ccccc5c34)nc(-c3cccc4oc5ccccc5c34)n1)c1ccccc12 ; c1ccc(-c2ccc(-c3nc(-c4ccc(-c5cccc6oc7ccccc7c56)cc4)nc(-c4cccc5oc6ccccc6c45)n3)cc2)cc1]

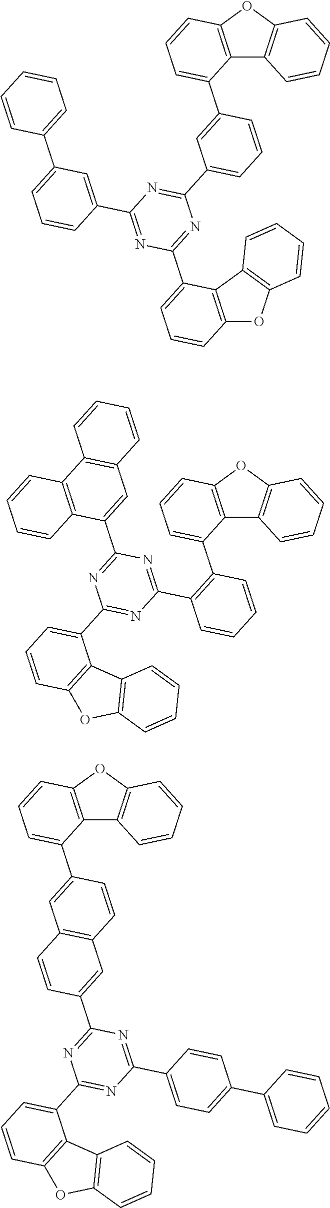 [SMILES: c1ccc(-c2cccc(-c3nc(-c4cccc(-c5cccc6oc7ccccc7c56)c4)nc(-c4cccc5oc6ccccc6c45)n3)c2)cc1 ; c1ccc(-c2cccc3oc4ccccc4c23)c(-c2nc(-c3cc4ccccc4c4ccccc34)nc(-c3cccc4oc5ccccc5c34)n2)c1 ; c1ccc(-c2ccc(-c3nc(-c4ccc5cc(-c6cccc7oc8ccccc8c67)ccc5c4)nc(-c4cccc5oc6ccccc6c45)n3)cc2)cc1]

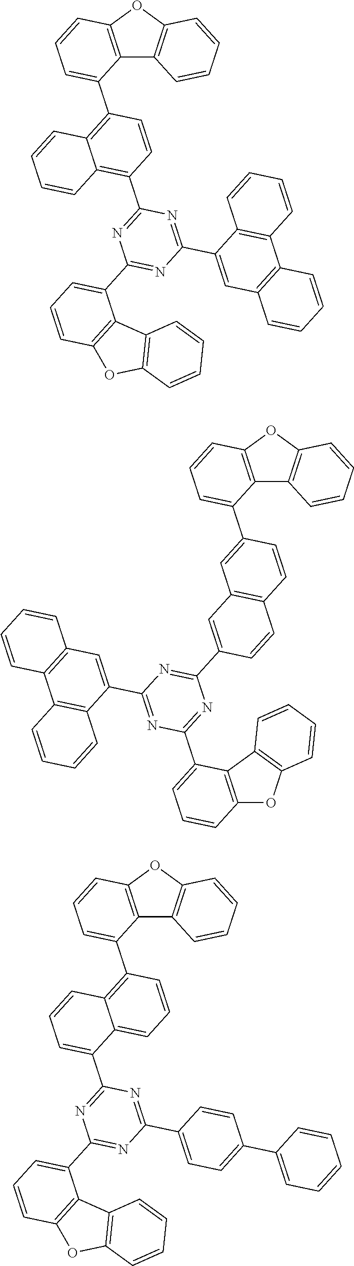 [SMILES: c1ccc2c(c1)cc(-c1nc(-c3ccc(-c4cccc5oc6ccccc6c45)c4ccccc34)nc(-c3cccc4oc5ccccc5c34)n1)c1ccccc12 ; c1ccc2c(c1)cc(-c1nc(-c3ccc4ccc(-c5cccc6oc7ccccc7c56)cc4c3)nc(-c3cccc4oc5ccccc5c34)n1)c1ccccc12 ; c1ccc(-c2ccc(-c3nc(-c4cccc5c(-c6cccc7oc8ccccc8c67)cccc45)nc(-c4cccc5oc6ccccc6c45)n3)cc2)cc1]

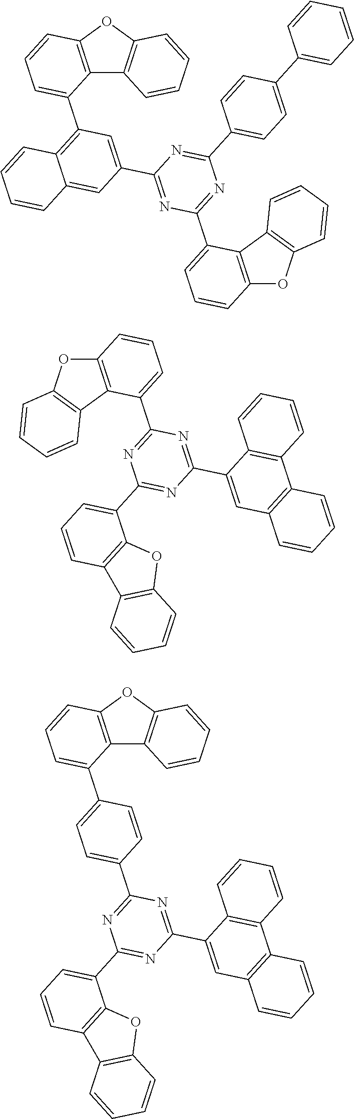

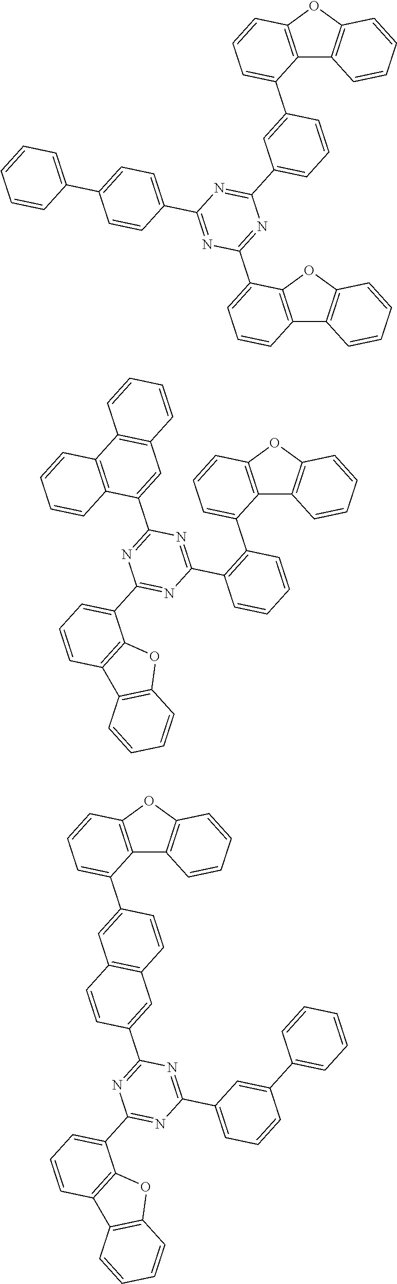 [SMILES: c1ccc(-c2ccc(-c3nc(-c4cccc(-c5cccc6oc7ccccc7c56)c4)nc(-c4cccc5c4oc4ccccc45)n3)cc2)cc1 ; c1ccc(-c2cccc3oc4ccccc4c23)c(-c2nc(-c3cc4ccccc4c4ccccc34)nc(-c3cccc4c3oc3ccccc34)n2)c1 ; c1ccc(-c2cccc(-c3nc(-c4ccc5cc(-c6cccc7oc8ccccc8c67)ccc5c4)nc(-c4cccc5c4oc4ccccc45)n3)c2)cc1]

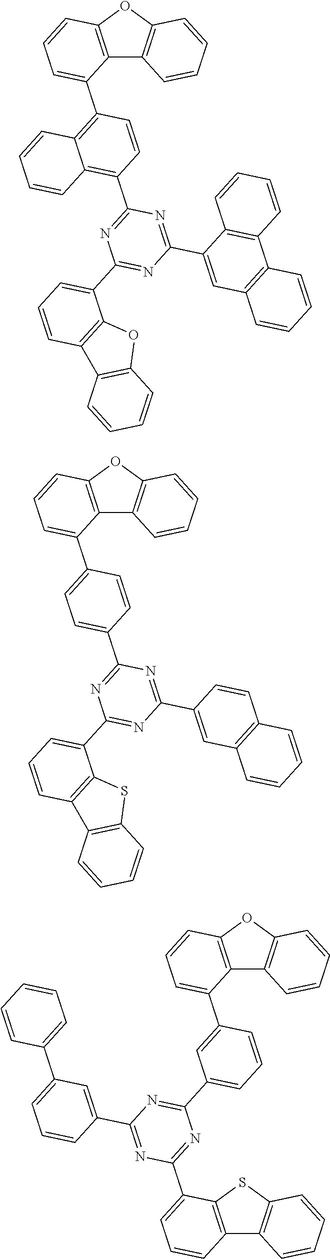

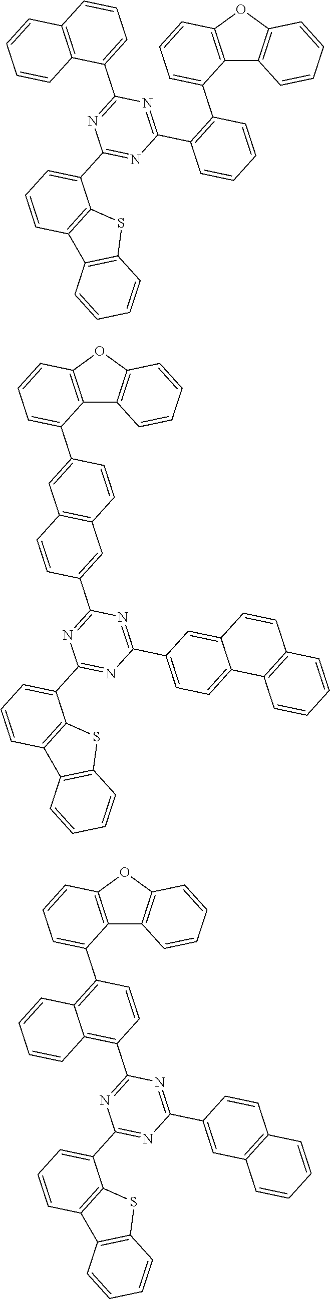

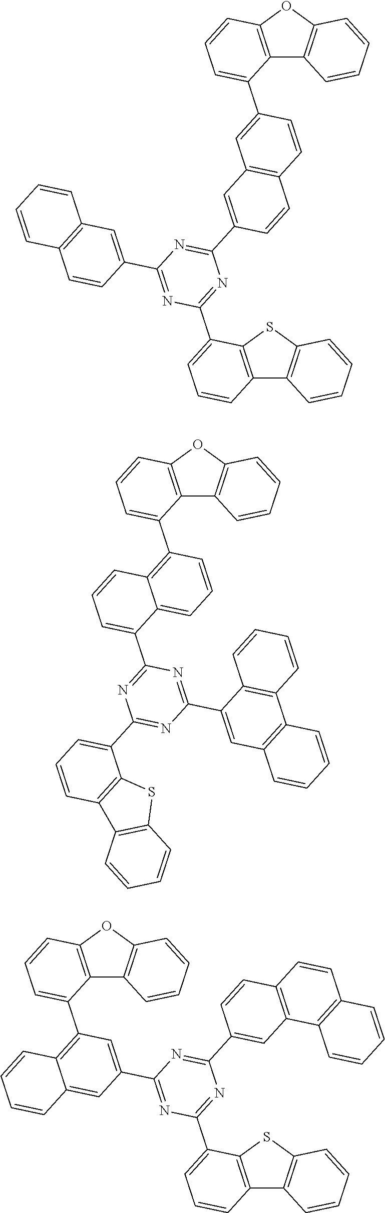

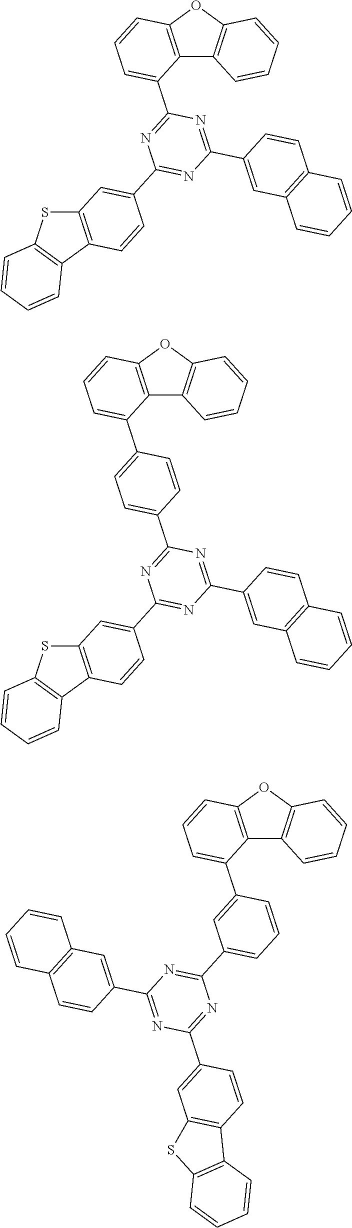 [SMILES: c1ccc2cc(-c3nc(-c4ccc5c(c4)sc4ccccc45)nc(-c4cccc5oc6ccccc6c45)n3)ccc2c1 ; c1ccc2cc(-c3nc(-c4ccc(-c5cccc6oc7ccccc7c56)cc4)nc(-c4ccc5c(c4)sc4ccccc45)n3)ccc2c1 ; c1cc(-c2nc(-c3ccc4ccccc4c3)nc(-c3ccc4c(c3)sc3ccccc34)n2)cc(-c2cccc3oc4ccccc4c23)c1]

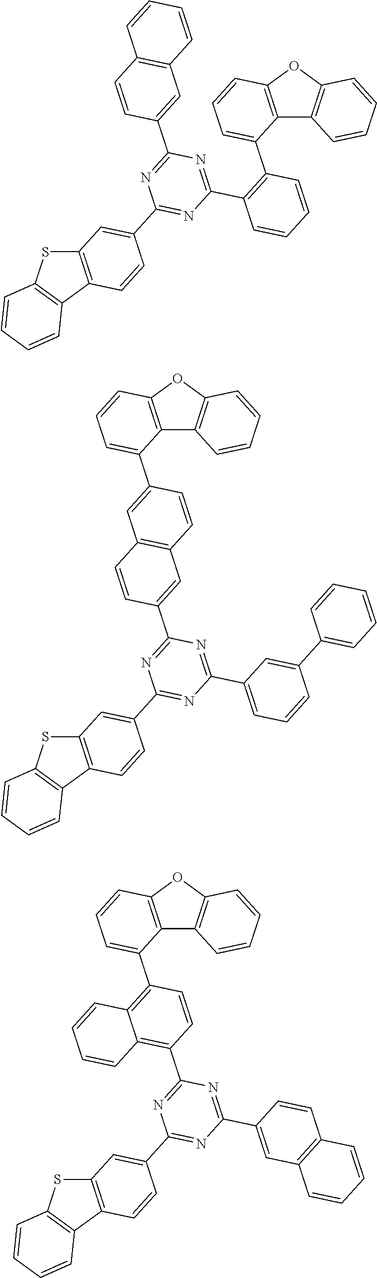 [SMILES: c1ccc(-c2cccc3oc4ccccc4c23)c(-c2nc(-c3ccc4ccccc4c3)nc(-c3ccc4c(c3)sc3ccccc34)n2)c1 ; c1ccc(-c2cccc(-c3nc(-c4ccc5cc(-c6cccc7oc8ccccc8c67)ccc5c4)nc(-c4ccc5c(c4)sc4ccccc45)n3)c2)cc1 ; c1ccc2cc(-c3nc(-c4ccc5c(c4)sc4ccccc45)nc(-c4ccc(-c5cccc6oc7ccccc7c56)c5ccccc45)n3)ccc2c1]

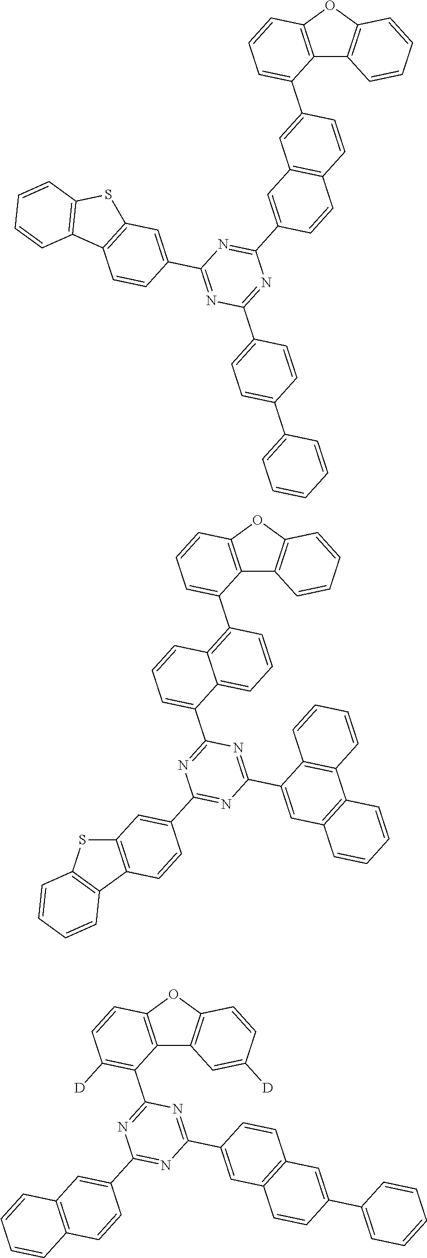

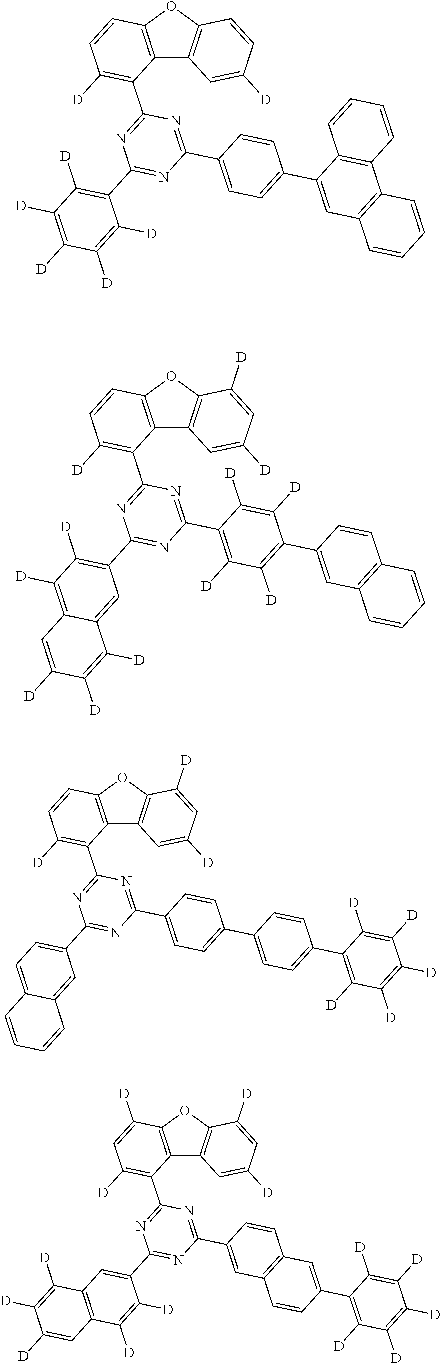

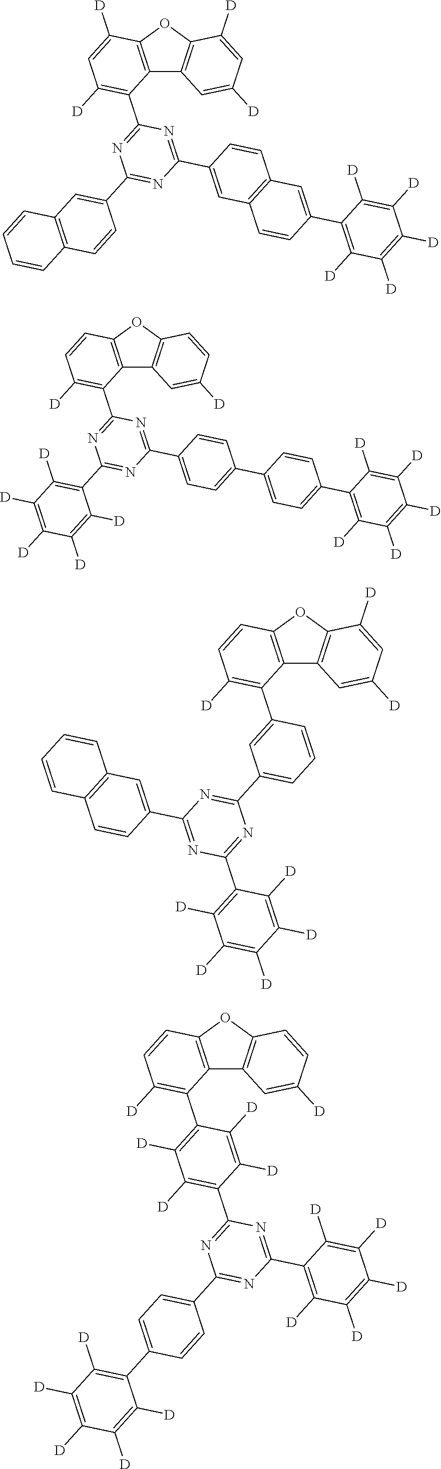

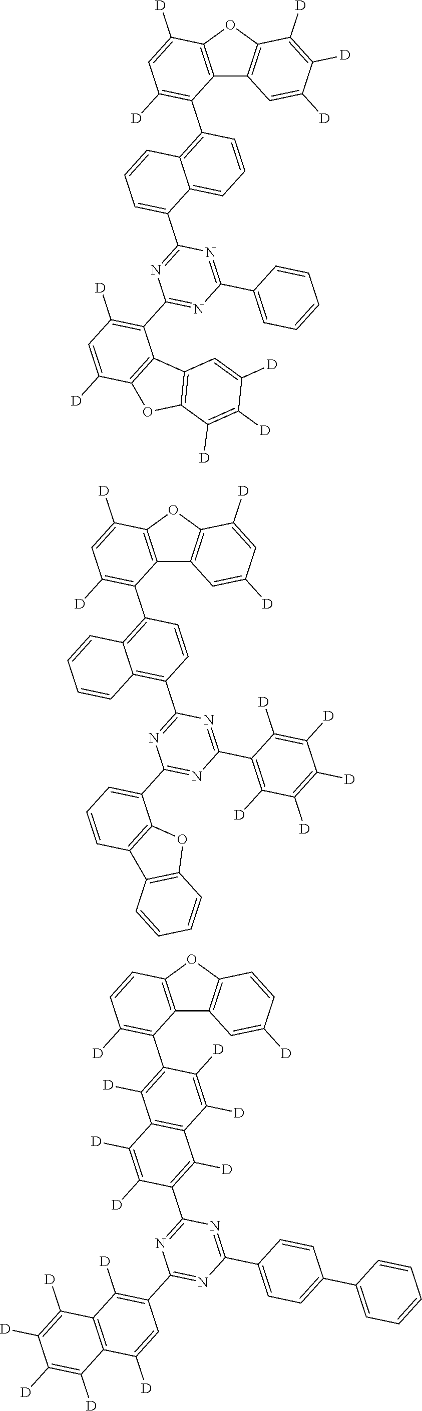

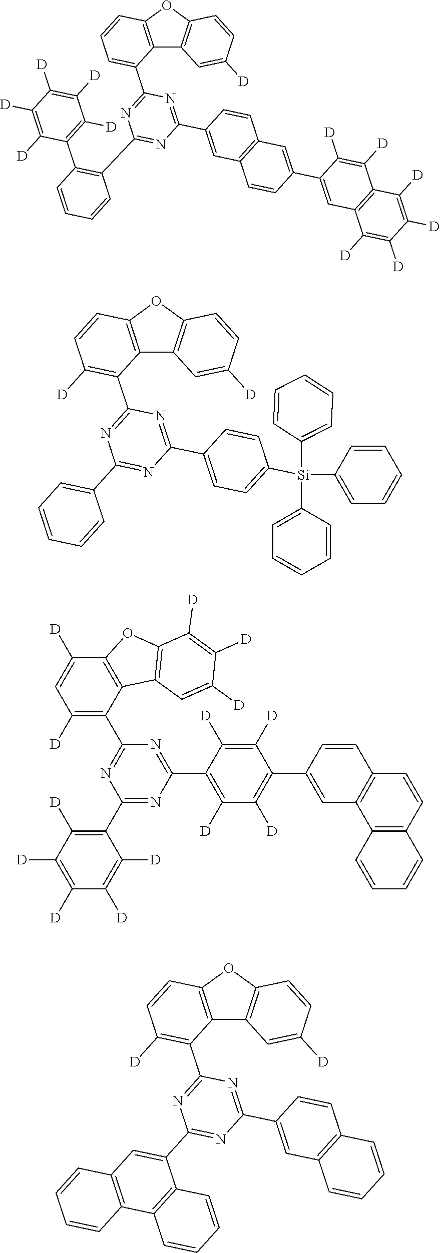 [SMILES: [2H]c1ccc2oc3cccc(-c4nc(-c5ccc6cc(-c7cc8c([2H])c([2H])c([2H])c([2H])c8c([2H])c7[2H])ccc6c5)nc(-c5ccccc5-c5c([2H])c([2H])c([2H])c([2H])c5[2H])n4)c3c2c1 ; [2H]c1ccc2oc3ccc([2H])c(-c4nc(-c5ccccc5)nc(-c5ccc([Si](c6ccccc6)(c6ccccc6)c6ccccc6)cc5)n4)c3c2c1 ; [2H]c1cc2c(oc3c([2H])cc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c32)c([2H])c1[2H] ; [2H]c1ccc2oc3ccc([2H])c(-c4nc(-c5ccc6ccccc6c5)nc(-c5cc6ccccc6c6ccccc56)n4)c3c2c1]

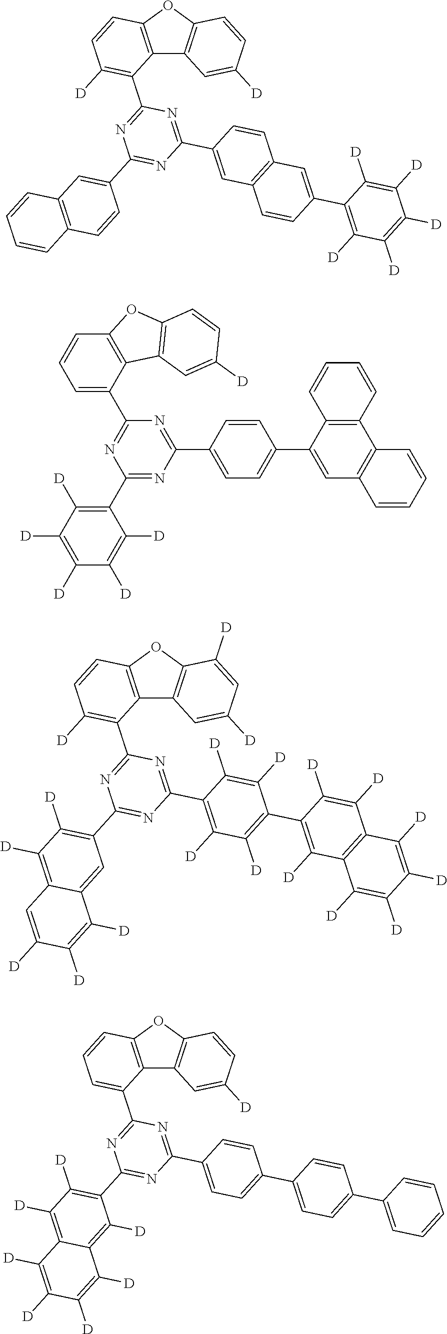

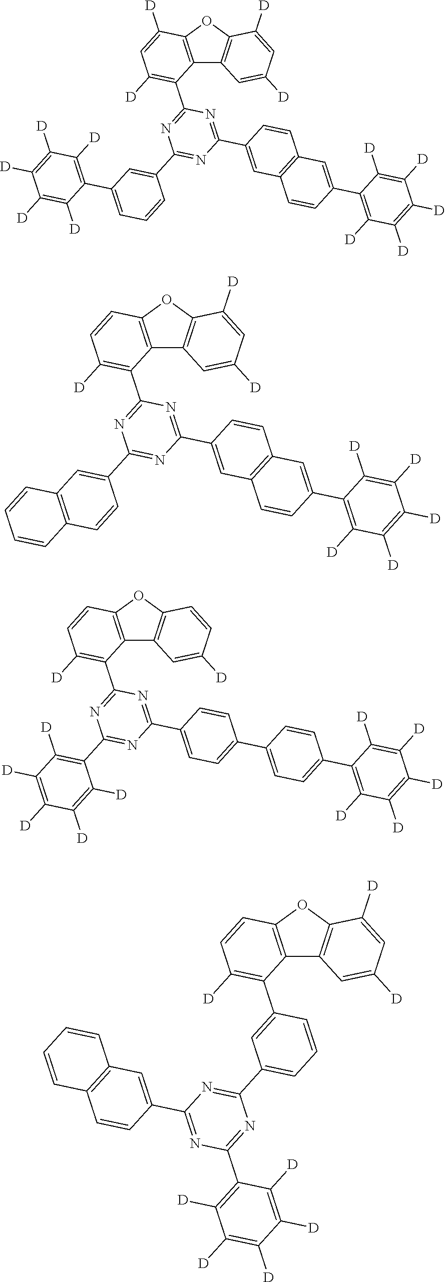

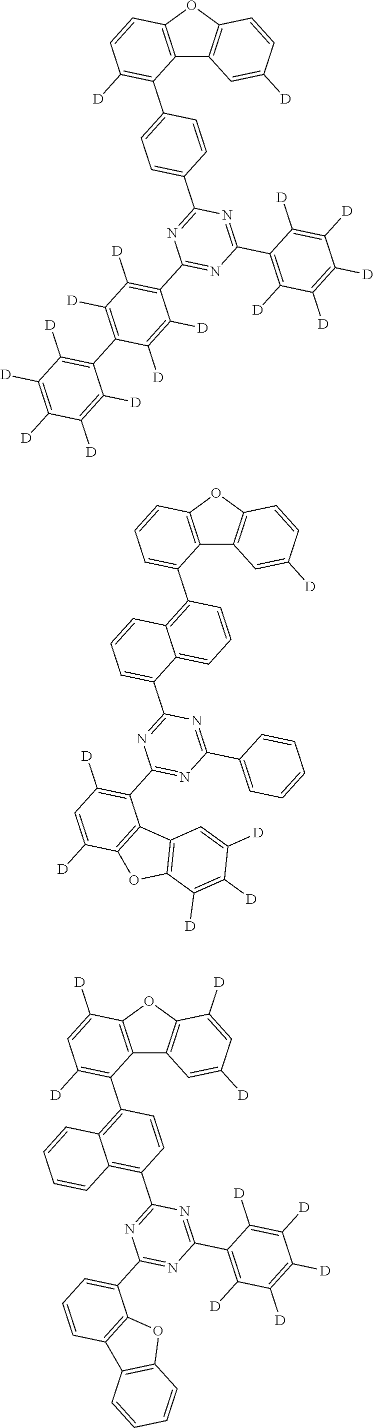 [SMILES: [2H]c1ccc2oc3ccc([2H])c(-c4ccc(-c5nc(-c6c([2H])c([2H])c([2H])c([2H])c6[2H])nc(-c6c([2H])c([2H])c(-c7c([2H])c([2H])c([2H])c([2H])c7[2H])c([2H])c6[2H])n5)cc4)c3c2c1 ; [2H]c1ccc2oc3cccc(-c4cccc5c(-c6nc(-c7ccccc7)nc(-c7c([2H])cc([2H])c8oc9c([2H])c([2H])c([2H])cc9c78)n6)cccc45)c3c2c1 ; [2H]c1cc([2H])c2oc3c([2H])cc([2H])c(-c4ccc(-c5nc(-c6c([2H])c([2H])c([2H])c([2H])c6[2H])nc(-c6cccc7c6oc6ccccc67)n5)c5ccccc45)c3c2c1]

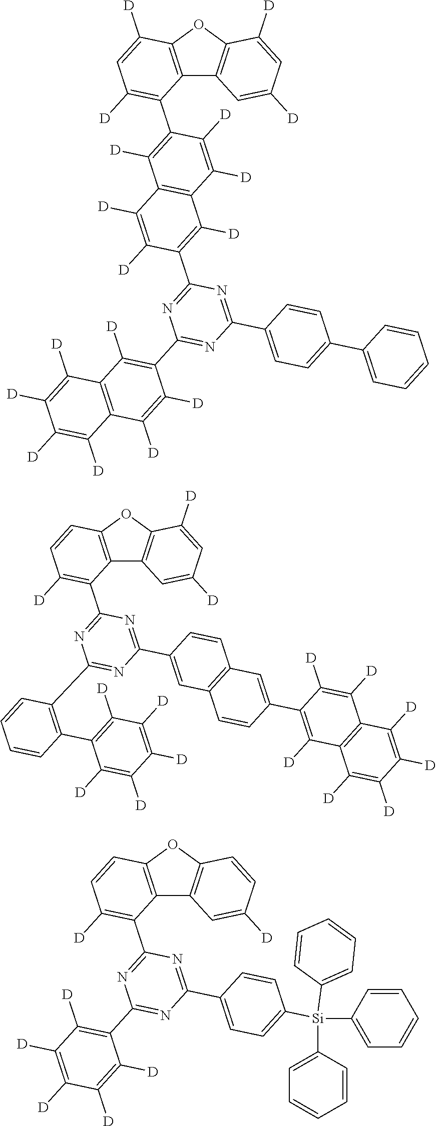 [SMILES: [2H]c1cc([2H])c2oc3c([2H])cc([2H])c(-c4c([2H])c([2H])c5c([2H])c(-c6nc(-c7ccc(-c8ccccc8)cc7)nc(-c7c([2H])c([2H])c8c([2H])c([2H])c([2H])c([2H])c8c7[2H])n6)c([2H])c([2H])c5c4[2H])c3c2c1 ; [2H]c1cc([2H])c2oc3ccc([2H])c(-c4nc(-c5ccc6cc(-c7c([2H])c([2H])c8c([2H])c([2H])c([2H])c([2H])c8c7[2H])ccc6c5)nc(-c5ccccc5-c5c([2H])c([2H])c([2H])c([2H])c5[2H])n4)c3c2c1 ; [2H]c1ccc2oc3ccc([2H])c(-c4nc(-c5ccc([Si](c6ccccc6)(c6ccccc6)c6ccccc6)cc5)nc(-c5c([2H])c([2H])c([2H])c([2H])c5[2H])n4)c3c2c1]

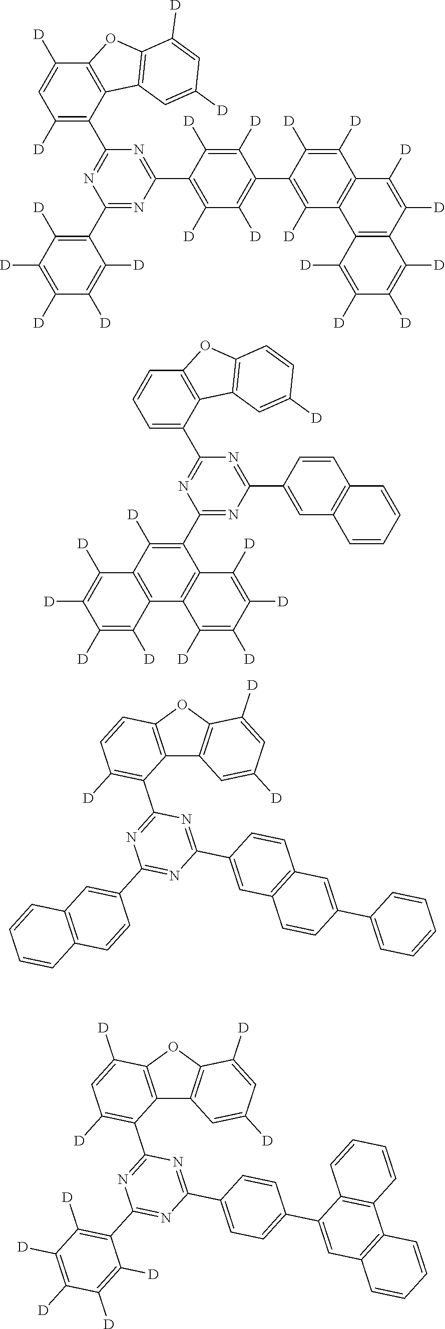

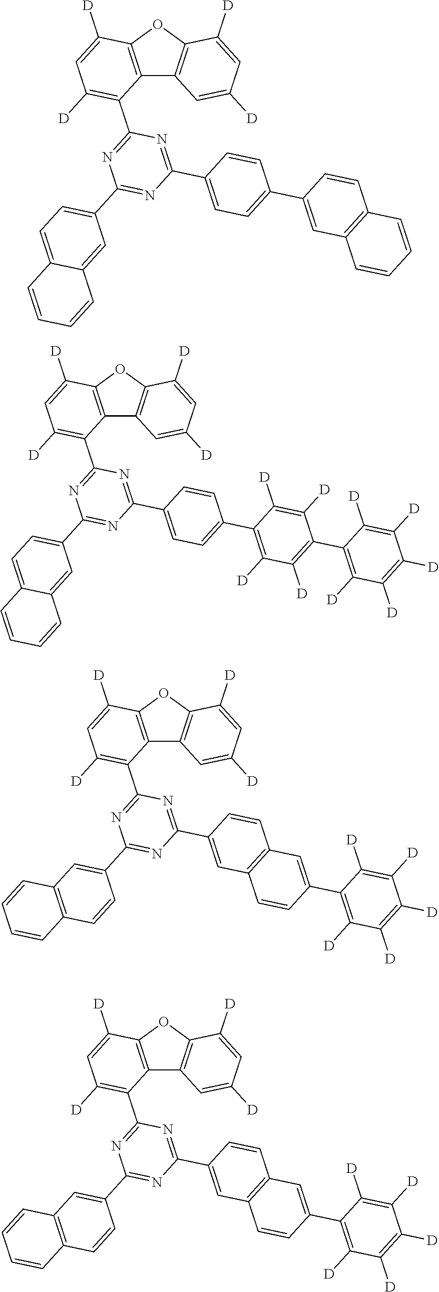

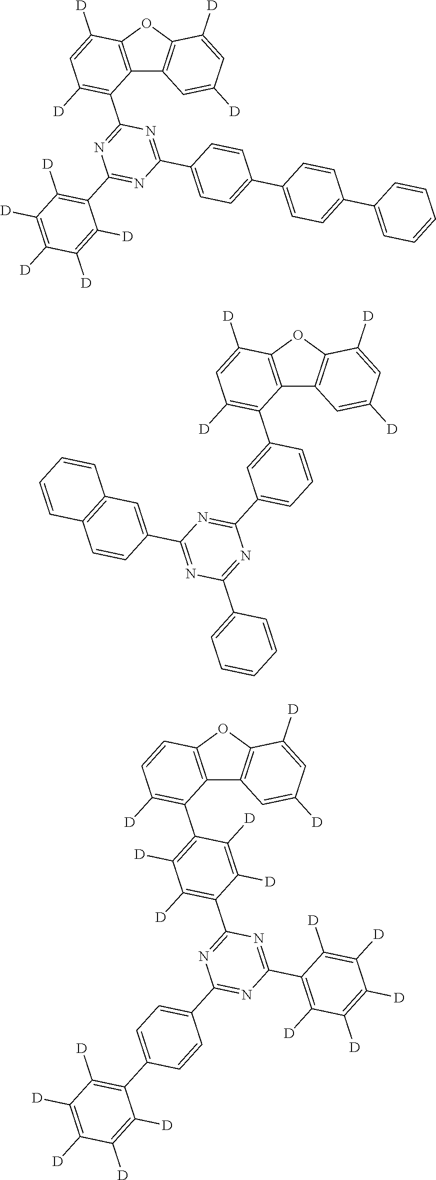 [SMILES: [2H]c1cc([2H])c2oc3c([2H])cc([2H])c(-c4nc(-c5ccc(-c6ccc(-c7ccccc7)cc6)cc5)nc(-c5c([2H])c([2H])c([2H])c([2H])c5[2H])n4)c3c2c1 ; [2H]c1cc([2H])c2oc3c([2H])cc([2H])c(-c4cccc(-c5nc(-c6ccccc6)nc(-c6ccc7ccccc7c6)n5)c4)c3c2c1 ; [2H]c1cc([2H])c2oc3ccc([2H])c(-c4c([2H])c([2H])c(-c5nc(-c6ccc(-c7c([2H])c([2H])c([2H])c([2H])c7[2H])cc6)nc(-c6c([2H])c([2H])c([2H])c([2H])c6[2H])n5)c([2H])c4[2H])c3c2c1]

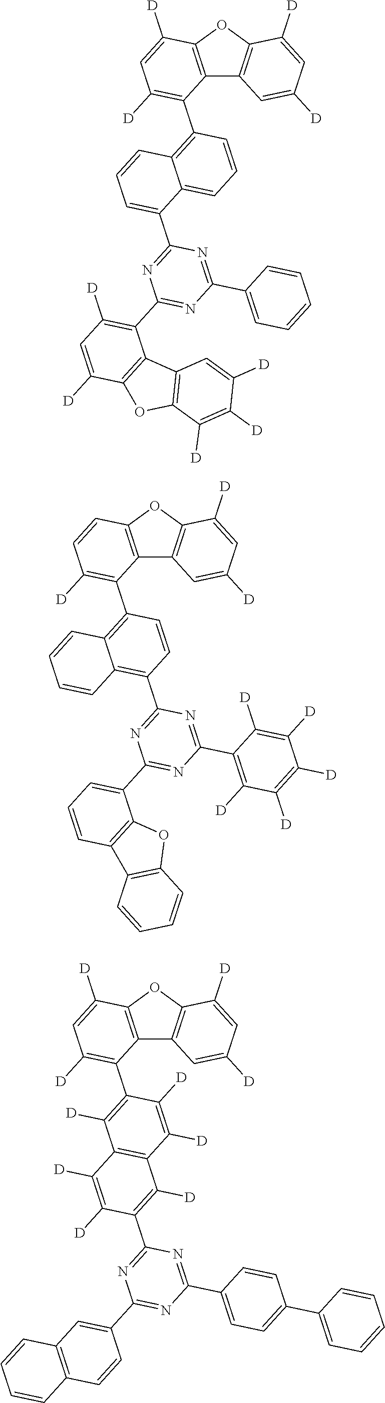

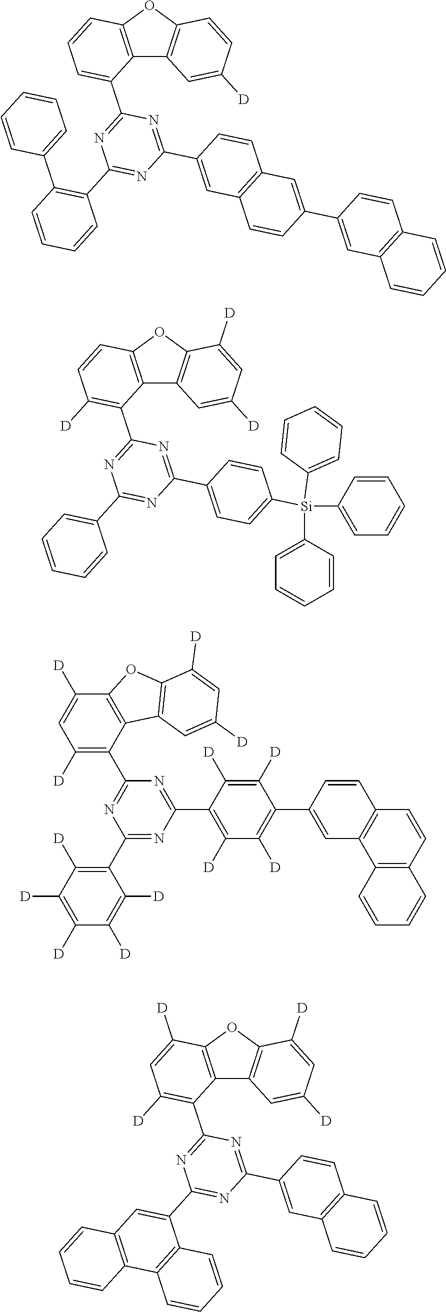 [SMILES: [2H]c1ccc2oc3cccc(-c4nc(-c5ccc6cc(-c7ccc8ccccc8c7)ccc6c5)nc(-c5ccccc5-c5ccccc5)n4)c3c2c1 ; [2H]c1cc([2H])c2oc3ccc([2H])c(-c4nc(-c5ccccc5)nc(-c5ccc([Si](c6ccccc6)(c6ccccc6)c6ccccc6)cc5)n4)c3c2c1 ; [2H]c1cc([2H])c2oc3c([2H])cc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c3c2c1 ; [2H]c1cc([2H])c2oc3c([2H])cc([2H])c(-c4nc(-c5ccc6ccccc6c5)nc(-c5cc6ccccc6c6ccccc56)n4)c3c2c1]

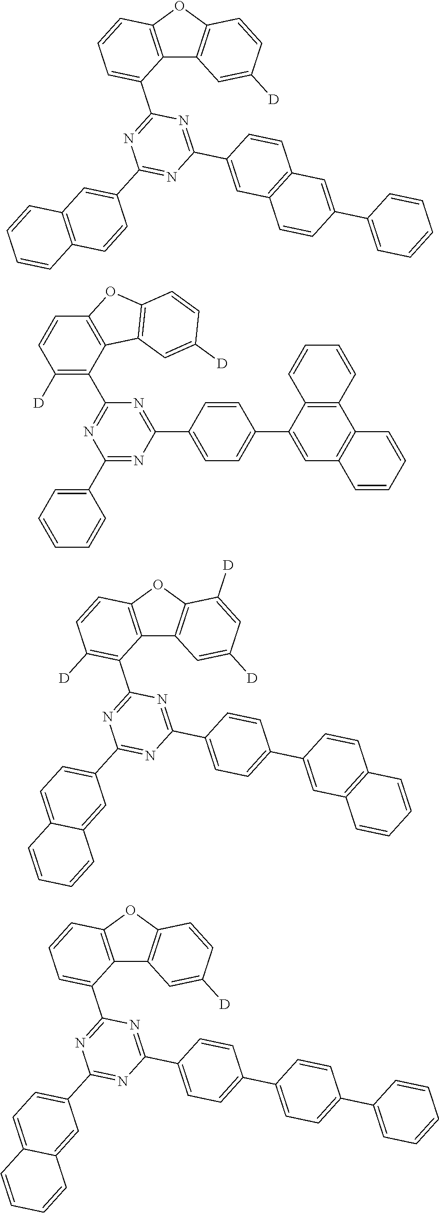

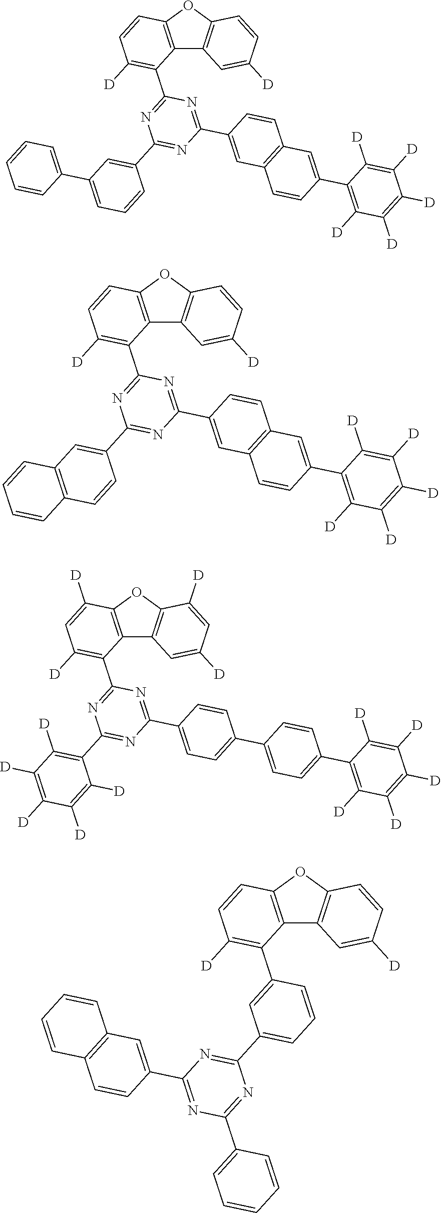 [SMILES: [2H]c1ccc2oc3ccc([2H])c(-c4nc(-c5cccc(-c6ccccc6)c5)nc(-c5ccc6cc(-c7c([2H])c([2H])c([2H])c([2H])c7[2H])ccc6c5)n4)c3c2c1 ; [2H]c1ccc2oc3ccc([2H])c(-c4nc(-c5ccc6ccccc6c5)nc(-c5ccc6cc(-c7c([2H])c([2H])c([2H])c([2H])c7[2H])ccc6c5)n4)c3c2c1 ; [2H]c1cc([2H])c2oc3c([2H])cc([2H])c(-c4nc(-c5ccc(-c6ccc(-c7c([2H])c([2H])c([2H])c([2H])c7[2H])cc6)cc5)nc(-c5c([2H])c([2H])c([2H])c([2H])c5[2H])n4)c3c2c1 ; [2H]c1ccc2oc3ccc([2H])c(-c4cccc(-c5nc(-c6ccccc6)nc(-c6ccc7ccccc7c6)n5)c4)c3c2c1]

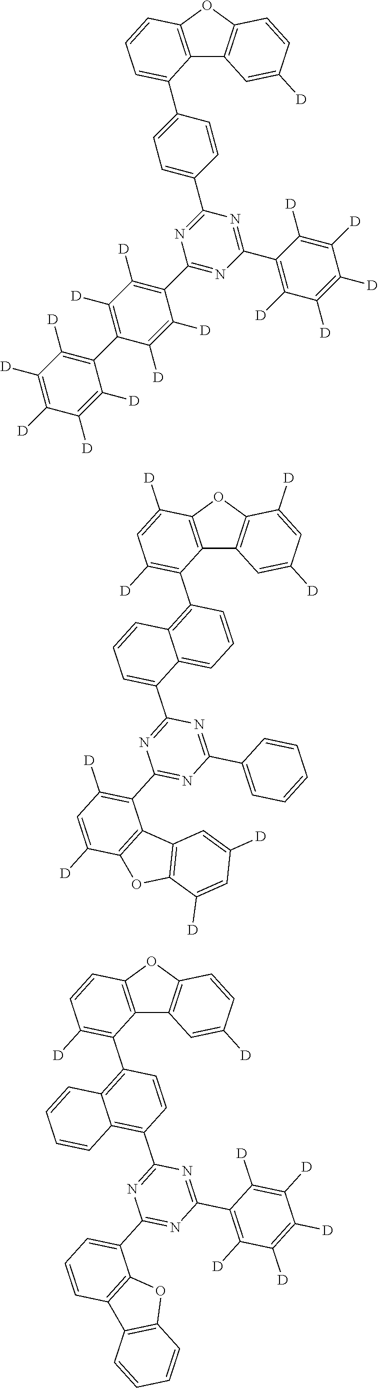 [SMILES: [2H]c1ccc2oc3cccc(-c4ccc(-c5nc(-c6c([2H])c([2H])c([2H])c([2H])c6[2H])nc(-c6c([2H])c([2H])c(-c7c([2H])c([2H])c([2H])c([2H])c7[2H])c([2H])c6[2H])n5)cc4)c3c2c1 ; [2H]c1cc([2H])c2oc3c([2H])cc([2H])c(-c4nc(-c5ccccc5)nc(-c5cccc6c(-c7c([2H])cc([2H])c8oc9c([2H])cc([2H])cc9c78)cccc56)n4)c3c2c1 ; [2H]c1ccc2oc3ccc([2H])c(-c4ccc(-c5nc(-c6c([2H])c([2H])c([2H])c([2H])c6[2H])nc(-c6cccc7c6oc6ccccc67)n5)c5ccccc45)c3c2c1]

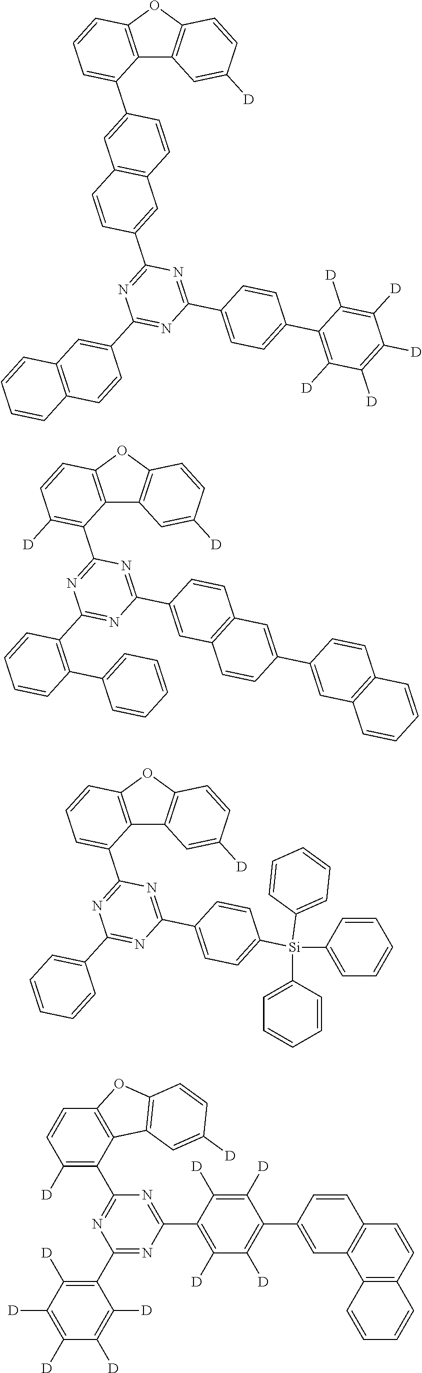 [SMILES: [2H]c1ccc2oc3cccc(-c4ccc5cc(-c6nc(-c7ccc(-c8c([2H])c([2H])c([2H])c([2H])c8[2H])cc7)nc(-c7ccc8ccccc8c7)n6)ccc5c4)c3c2c1 ; [2H]c1ccc2oc3ccc([2H])c(-c4nc(-c5ccc6cc(-c7ccc8ccccc8c7)ccc6c5)nc(-c5ccccc5-c5ccccc5)n4)c3c2c1 ; [2H]c1ccc2oc3cccc(-c4nc(-c5ccccc5)nc(-c5ccc([Si](c6ccccc6)(c6ccccc6)c6ccccc6)cc5)n4)c3c2c1 ; [2H]c1ccc2oc3ccc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c3c2c1]

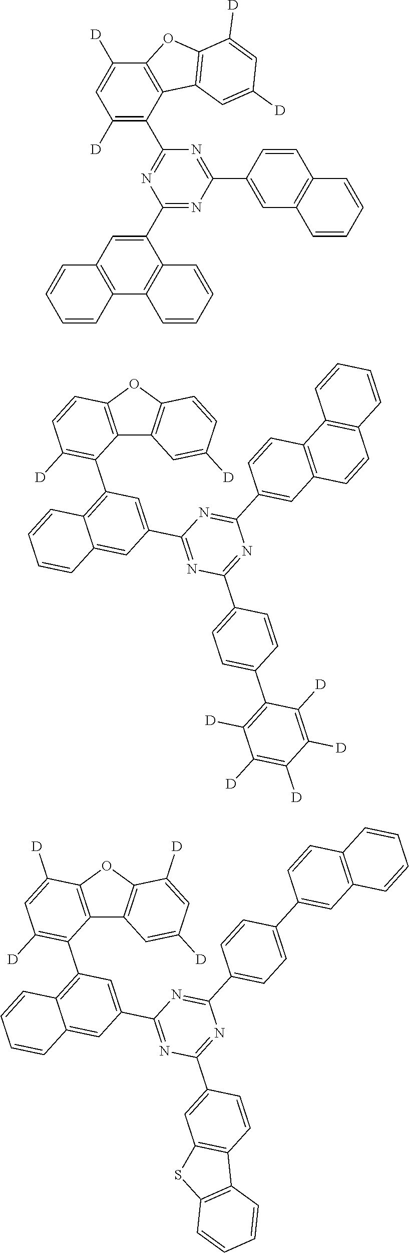 [SMILES: [2H]c1cc([2H])c2oc3c([2H])cc([2H])c(-c4nc(-c5ccc6ccccc6c5)nc(-c5cc6ccccc6c6ccccc56)n4)c3c2c1 ; [2H]c1ccc2oc3ccc([2H])c(-c4cc(-c5nc(-c6ccc(-c7c([2H])c([2H])c([2H])c([2H])c7[2H])cc6)nc(-c6ccc7c(ccc8ccccc87)c6)n5)cc5ccccc45)c3c2c1 ; [2H]c1cc([2H])c2oc3c([2H])cc([2H])c(-c4cc(-c5nc(-c6ccc(-c7ccc8ccccc8c7)cc6)nc(-c6ccc7c(c6)sc6ccccc67)n5)cc5ccccc45)c3c2c1]

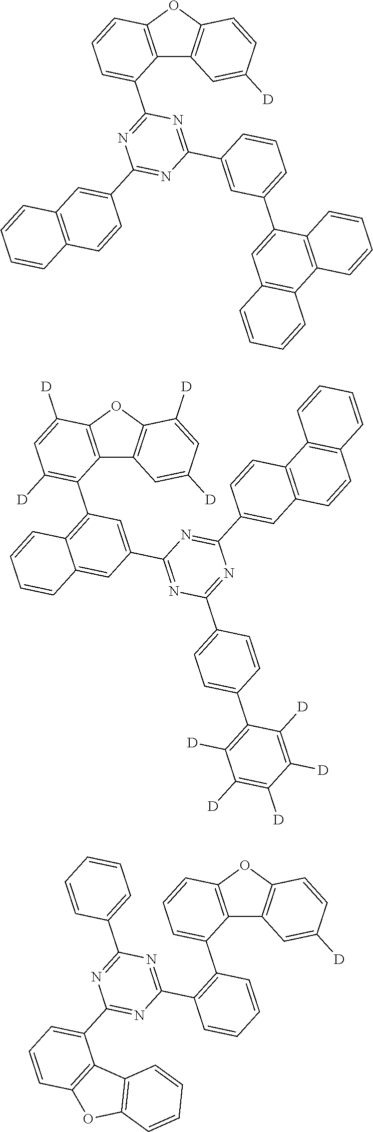 [SMILES: [2H]c1ccc2oc3cccc(-c4nc(-c5cccc(-c6cc7ccccc7c7ccccc67)c5)nc(-c5ccc6ccccc6c5)n4)c3c2c1 ; [2H]c1cc([2H])c2oc3c([2H])cc([2H])c(-c4cc(-c5nc(-c6ccc(-c7c([2H])c([2H])c([2H])c([2H])c7[2H])cc6)nc(-c6ccc7c(ccc8ccccc87)c6)n5)cc5ccccc45)c3c2c1 ; [2H]c1ccc2oc3cccc(-c4ccccc4-c4nc(-c5ccccc5)nc(-c5cccc6oc7ccccc7c56)n4)c3c2c1]

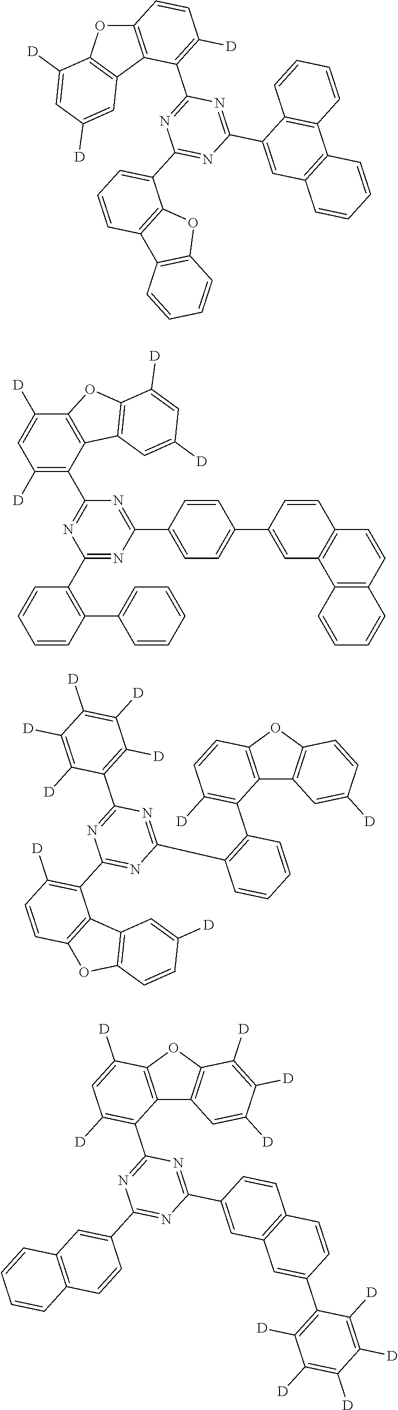

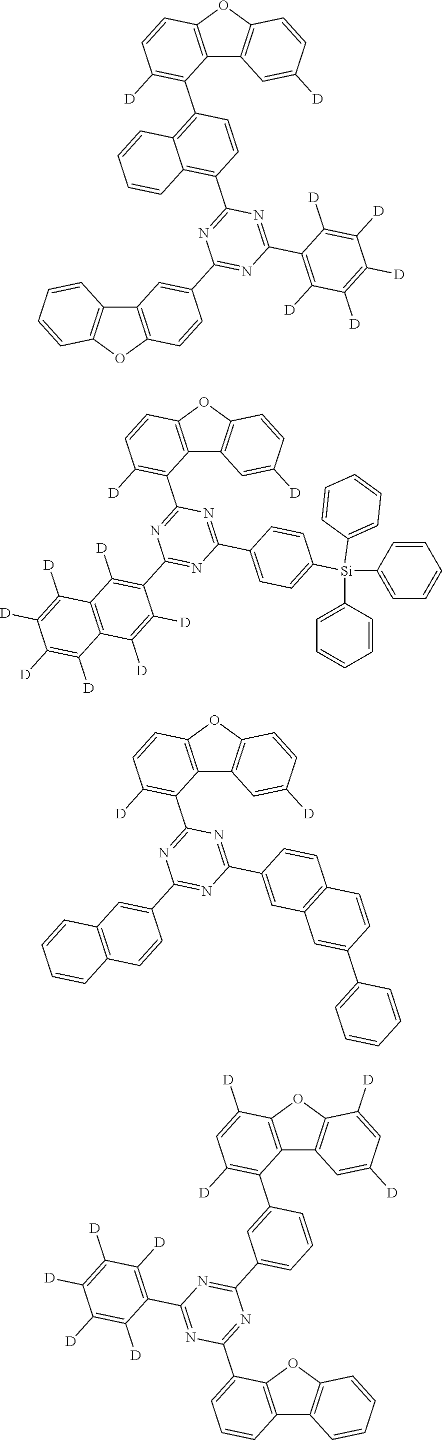

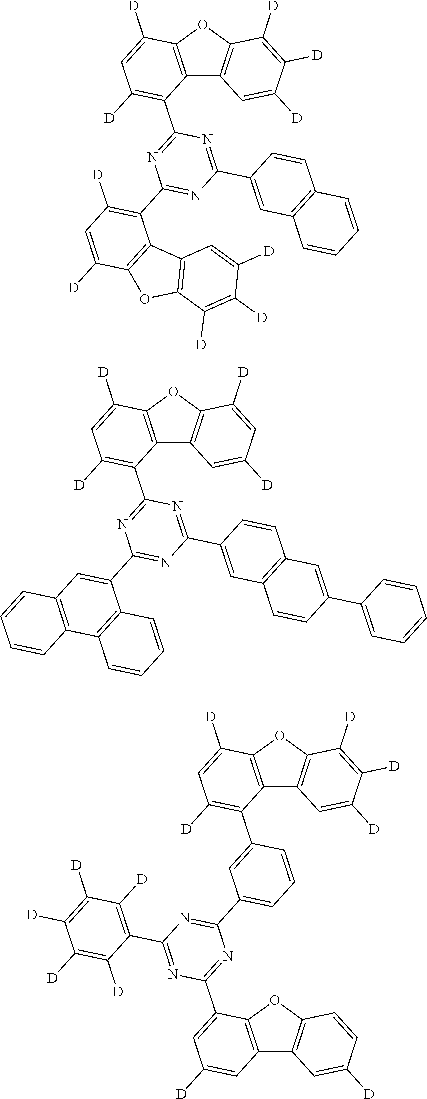 [SMILES: [2H]c1cc2c(oc3c([2H])cc([2H])c(-c4nc(-c5ccc6ccccc6c5)nc(-c5c([2H])cc([2H])c6oc7c([2H])c([2H])c([2H])cc7c56)n4)c32)c([2H])c1[2H] ; [2H]c1cc([2H])c2oc3c([2H])cc([2H])c(-c4nc(-c5ccc6cc(-c7ccccc7)ccc6c5)nc(-c5cc6ccccc6c6ccccc56)n4)c3c2c1 ; [2H]c1ccc2oc3c(-c4nc(-c5cccc(-c6c([2H])cc([2H])c7oc8c([2H])c([2H])c([2H])cc8c67)c5)nc(-c5c([2H])c([2H])c([2H])c([2H])c5[2H])n4)cc([2H])cc3c2c1]

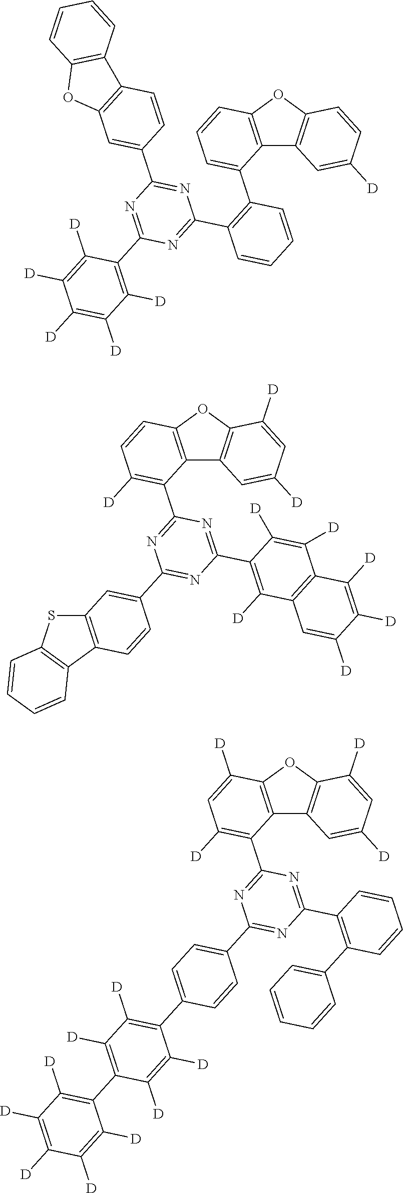 [SMILES: [2H]c1ccc2oc3cccc(-c4ccccc4-c4nc(-c5ccc6c(c5)oc5ccccc56)nc(-c5c([2H])c([2H])c([2H])c([2H])c5[2H])n4)c3c2c1 ; [2H]c1cc([2H])c2oc3ccc([2H])c(-c4nc(-c5ccc6c(c5)sc5ccccc56)nc(-c5c([2H])c([2H])c6c([2H])c([2H])c([2H])cc6c5[2H])n4)c3c2c1 ; [2H]c1cc([2H])c2oc3c([2H])cc([2H])c(-c4nc(-c5ccc(-c6c([2H])c([2H])c(-c7c([2H])c([2H])c([2H])c([2H])c7[2H])c([2H])c6[2H])cc5)nc(-c5ccccc5-c5ccccc5)n4)c3c2c1]

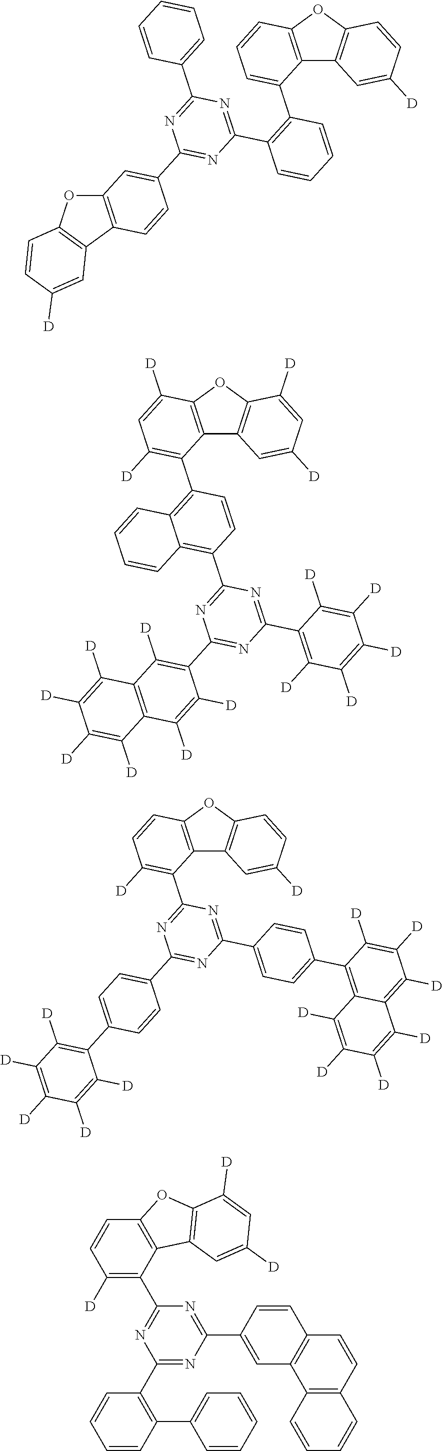

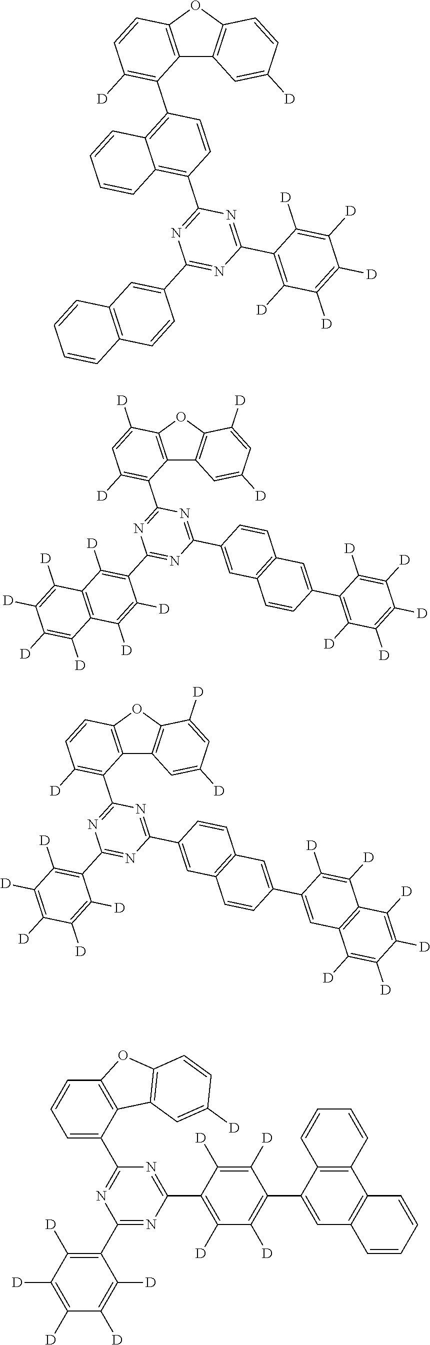

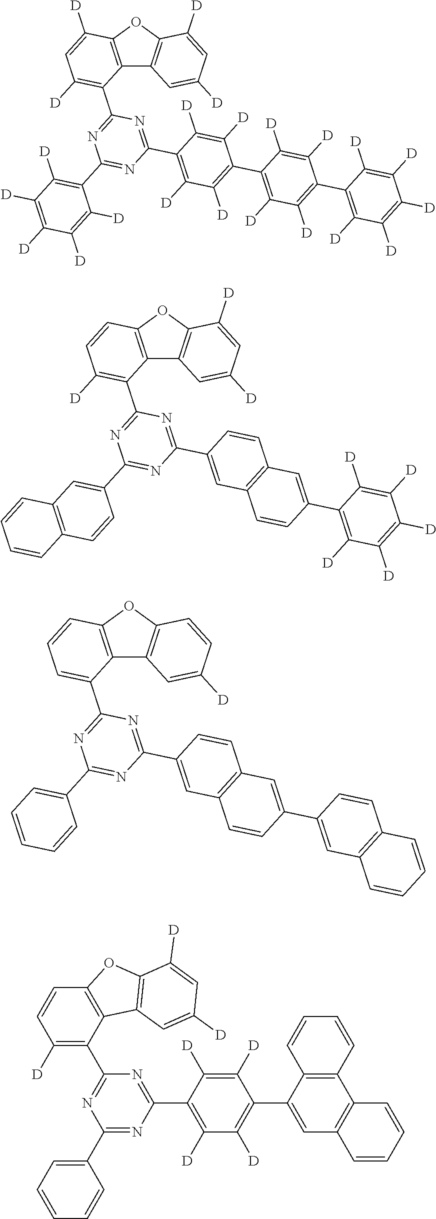

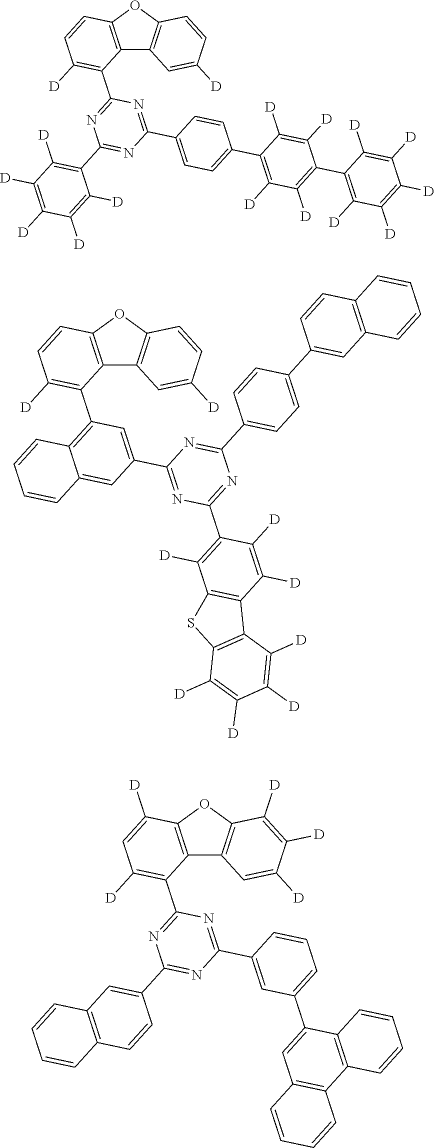

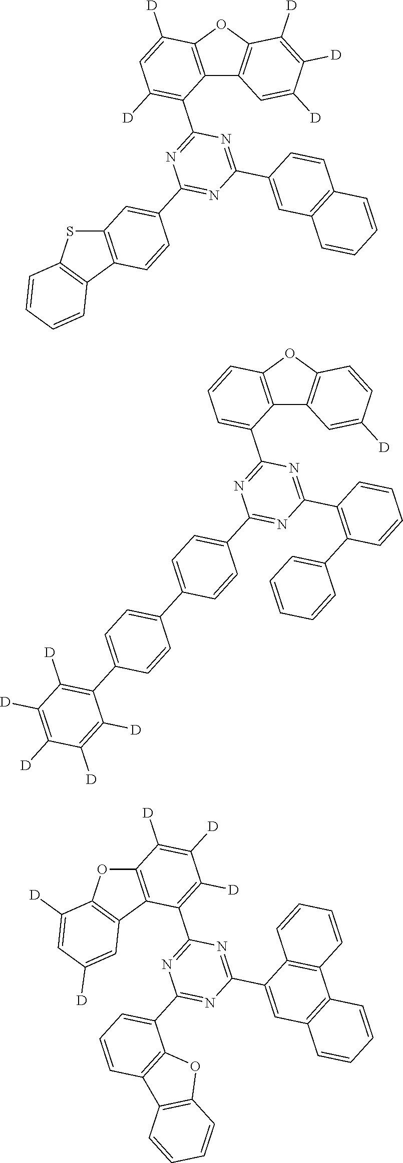 [SMILES: [2H]c1cc2c(oc3c([2H])cc([2H])c(-c4nc(-c5ccc6ccccc6c5)nc(-c5ccc6c(c5)sc5ccccc56)n4)c32)c([2H])c1[2H] ; [2H]c1ccc2oc3cccc(-c4nc(-c5ccc(-c6ccc(-c7c([2H])c([2H])c([2H])c([2H])c7[2H])cc6)cc5)nc(-c5ccccc5-c5ccccc5)n4)c3c2c1 ; [2H]c1cc([2H])c2oc3c([2H])c([2H])c([2H])c(-c4nc(-c5cc6ccccc6c6ccccc56)nc(-c5cccc6c5oc5ccccc56)n4)c3c2c1]

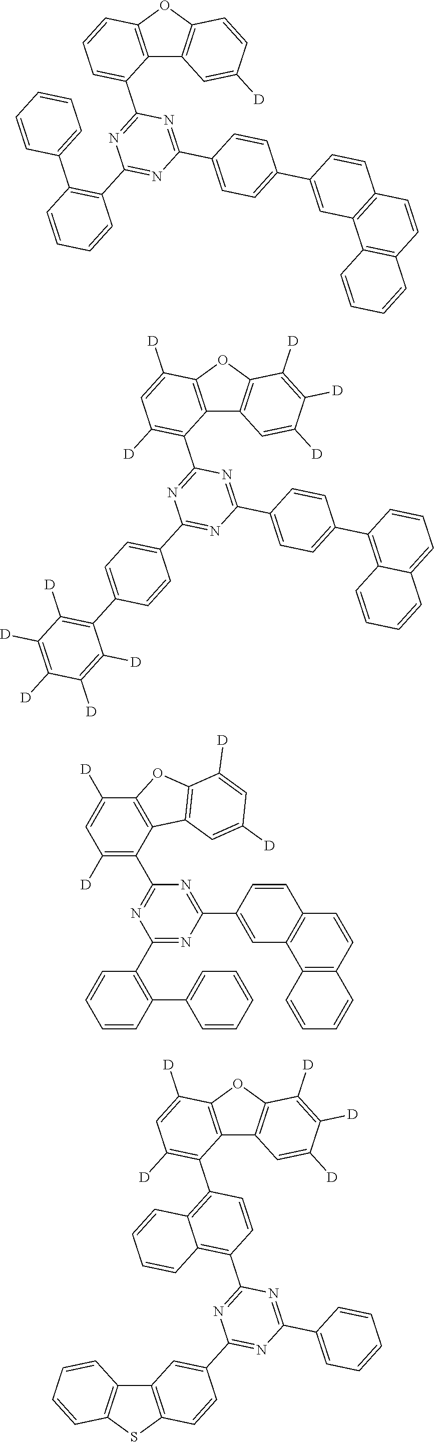

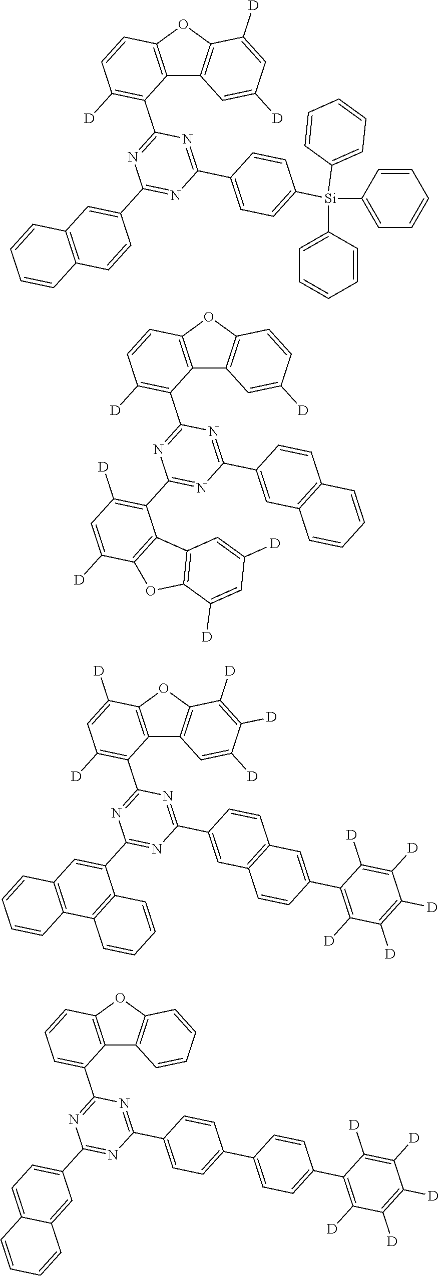

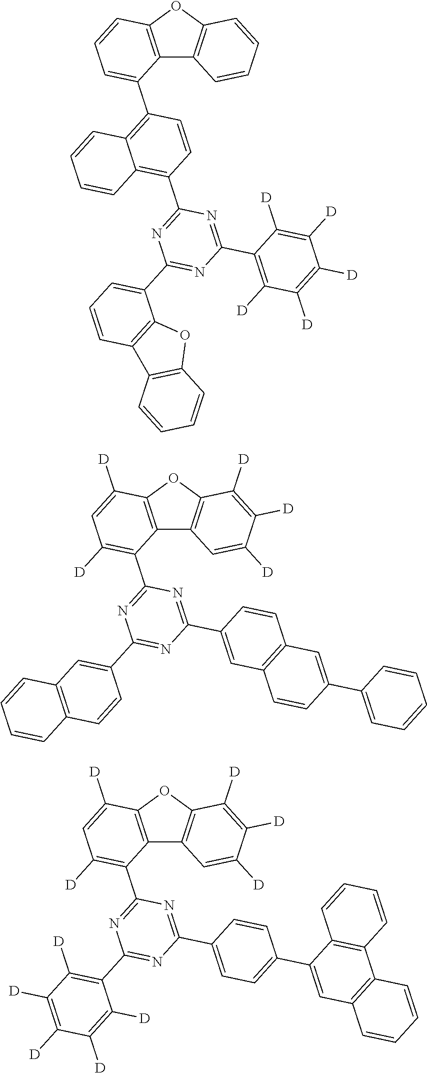 [SMILES: [2H]c1c([2H])c([2H])c(-c2nc(-c3ccc(-c4cccc5oc6ccccc6c45)c4ccccc34)nc(-c3cccc4c3oc3ccccc34)n2)c([2H])c1[2H] ; [2H]c1cc2c(oc3c([2H])cc([2H])c(-c4nc(-c5ccc6ccccc6c5)nc(-c5ccc6cc(-c7ccccc7)ccc6c5)n4)c32)c([2H])c1[2H] ; [2H]c1cc2c(oc3c([2H])cc([2H])c(-c4nc(-c5ccc(-c6cc7ccccc7c7ccccc67)cc5)nc(-c5c([2H])c([2H])c([2H])c([2H])c5[2H])n4)c32)c([2H])c1[2H]]

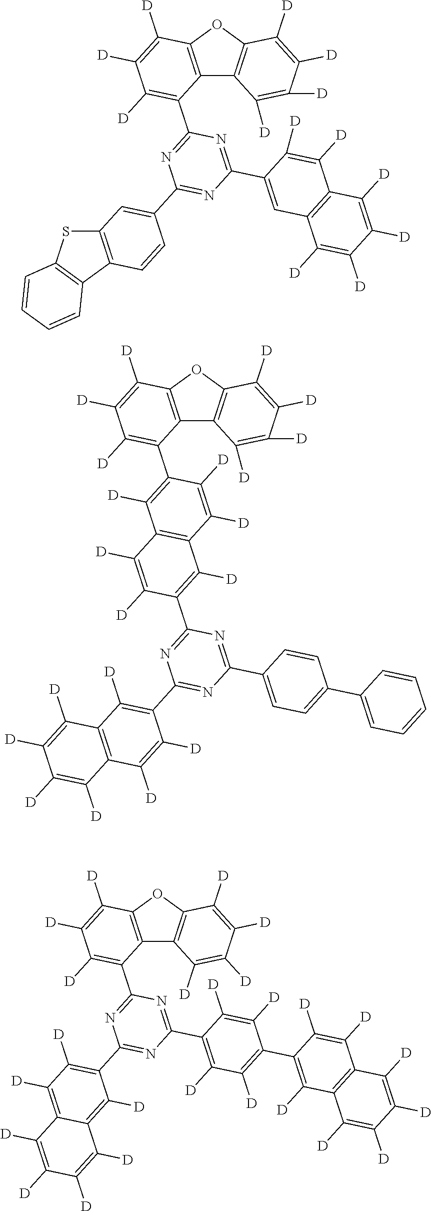

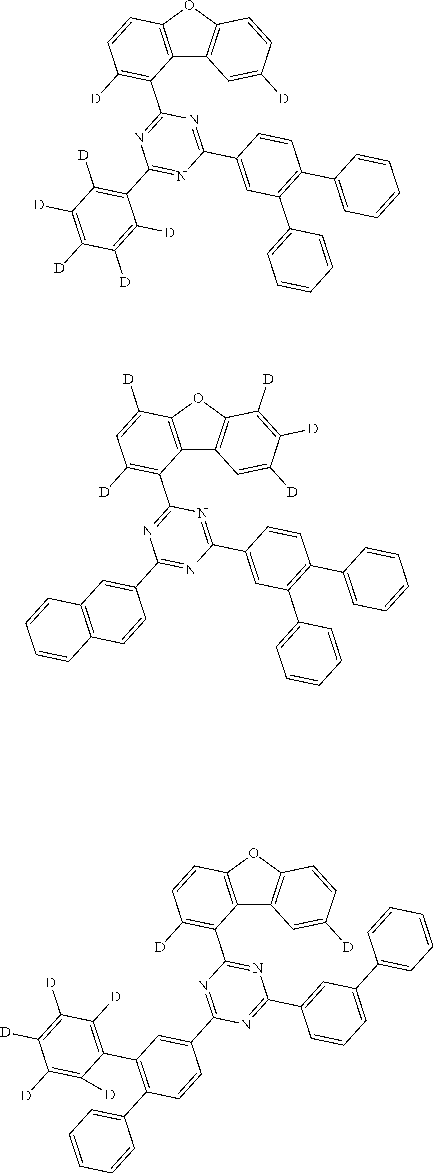 [SMILES: [2H]c1ccc2oc3ccc([2H])c(-c4nc(-c5ccc(-c6ccccc6)c(-c6ccccc6)c5)nc(-c5c([2H])c([2H])c([2H])c([2H])c5[2H])n4)c3c2c1 ; [2H]c1cc2c(oc3c([2H])cc([2H])c(-c4nc(-c5ccc(-c6ccccc6)c(-c6ccccc6)c5)nc(-c5ccc6ccccc6c5)n4)c32)c([2H])c1[2H] ; [2H]c1ccc2oc3ccc([2H])c(-c4nc(-c5cccc(-c6ccccc6)c5)nc(-c5ccc(-c6ccccc6)c(-c6c([2H])c([2H])c([2H])c([2H])c6[2H])c5)n4)c3c2c1]

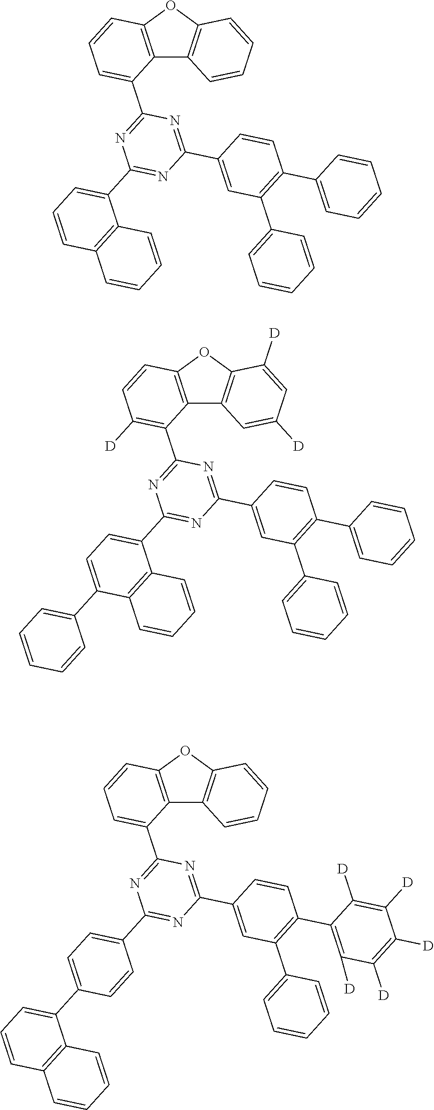

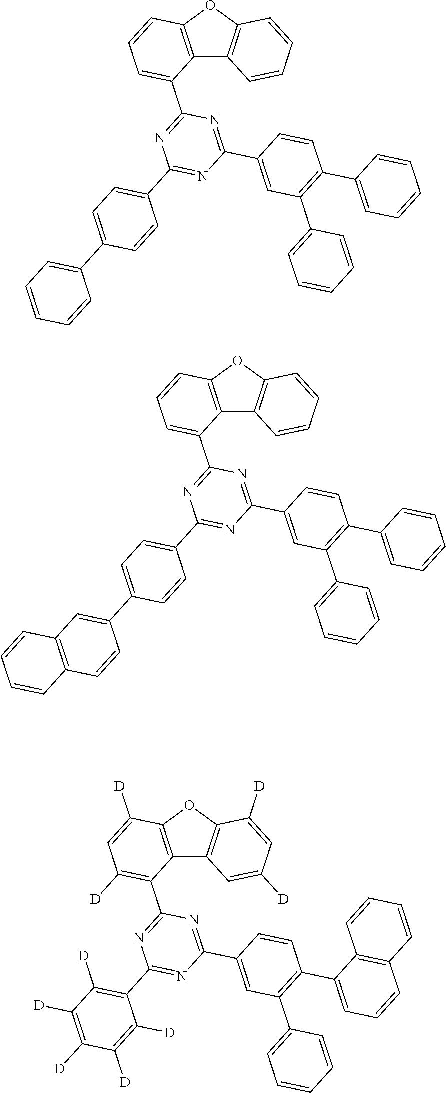 [SMILES: c1ccc(-c2ccc(-c3nc(-c4ccc(-c5ccccc5)c(-c5ccccc5)c4)nc(-c4cccc5oc6ccccc6c45)n3)cc2)cc1 ; c1ccc(-c2ccc(-c3nc(-c4ccc(-c5ccc6ccccc6c5)cc4)nc(-c4cccc5oc6ccccc6c45)n3)cc2-c2ccccc2)cc1 ; [2H]c1cc([2H])c2oc3c([2H])cc([2H])c(-c4nc(-c5ccc(-c6cccc7ccccc67)c(-c6ccccc6)c5)nc(-c5c([2H])c([2H])c([2H])c([2H])c5[2H])n4)c3c2c1]

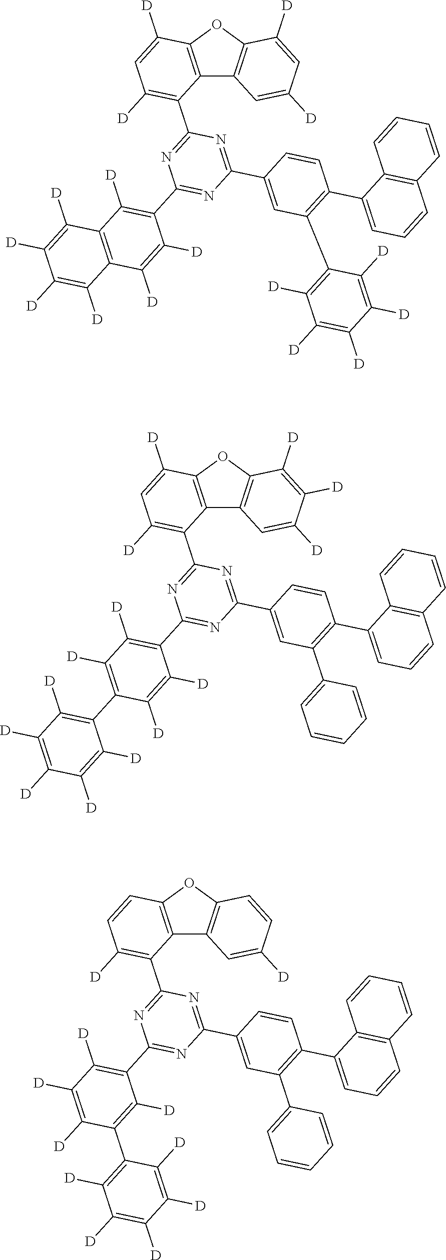

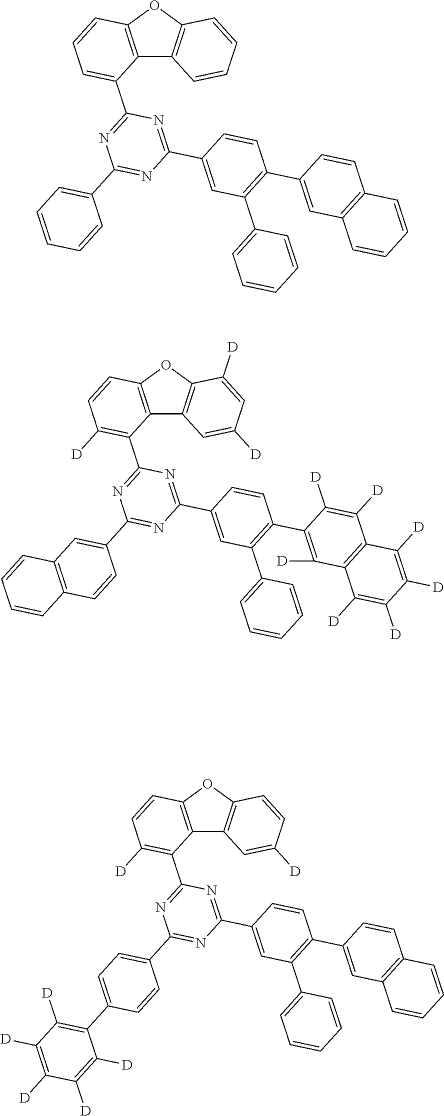 [SMILES: c1ccc(-c2nc(-c3ccc(-c4ccc5ccccc5c4)c(-c4ccccc4)c3)nc(-c3cccc4oc5ccccc5c34)n2)cc1 ; [2H]c1cc([2H])c2oc3ccc([2H])c(-c4nc(-c5ccc(-c6c([2H])c([2H])c7c([2H])c([2H])c([2H])c([2H])c7c6[2H])c(-c6ccccc6)c5)nc(-c5ccc6ccccc6c5)n4)c3c2c1 ; [2H]c1ccc2oc3ccc([2H])c(-c4nc(-c5ccc(-c6c([2H])c([2H])c([2H])c([2H])c6[2H])cc5)nc(-c5ccc(-c6ccc7ccccc7c6)c(-c6ccccc6)c5)n4)c3c2c1]

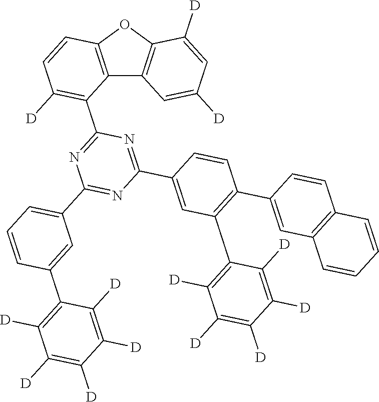 [SMILES: [2H]c1cc([2H])c2oc3ccc([2H])c(-c4nc(-c5cccc(-c6c([2H])c([2H])c([2H])c([2H])c6[2H])c5)nc(-c5ccc(-c6ccc7ccccc7c6)c(-c6c([2H])c([2H])c([2H])c([2H])c6[2H])c5)n4)c3c2c1]

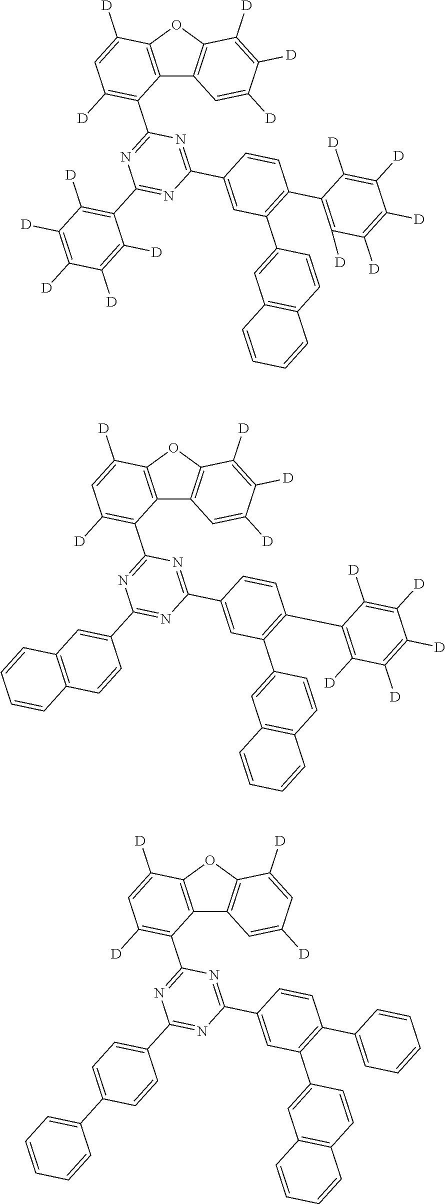 [SMILES: [2H]c1cc2c(oc3c([2H])cc([2H])c(-c4nc(-c5ccc(-c6c([2H])c([2H])c([2H])c([2H])c6[2H])c(-c6ccc7ccccc7c6)c5)nc(-c5c([2H])c([2H])c([2H])c([2H])c5[2H])n4)c32)c([2H])c1[2H] ; [2H]c1cc2c(oc3c([2H])cc([2H])c(-c4nc(-c5ccc(-c6c([2H])c([2H])c([2H])c([2H])c6[2H])c(-c6ccc7ccccc7c6)c5)nc(-c5ccc6ccccc6c5)n4)c32)c([2H])c1[2H] ; [2H]c1cc([2H])c2oc3c([2H])cc([2H])c(-c4nc(-c5ccc(-c6ccccc6)cc5)nc(-c5ccc(-c6ccccc6)c(-c6ccc7ccccc7c6)c5)n4)c3c2c1]

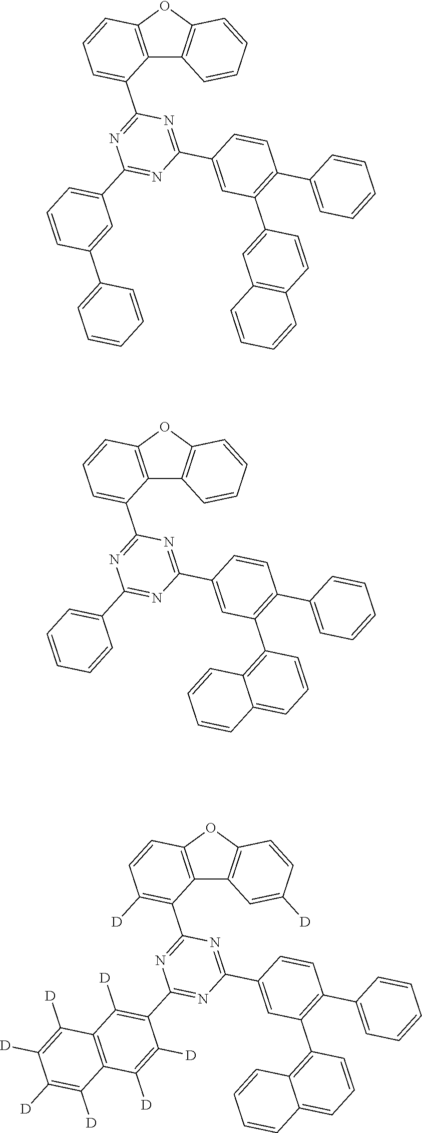 [SMILES: c1ccc(-c2cccc(-c3nc(-c4ccc(-c5ccccc5)c(-c5ccc6ccccc6c5)c4)nc(-c4cccc5oc6ccccc6c45)n3)c2)cc1 ; c1ccc(-c2nc(-c3ccc(-c4ccccc4)c(-c4cccc5ccccc45)c3)nc(-c3cccc4oc5ccccc5c34)n2)cc1 ; [2H]c1ccc2oc3ccc([2H])c(-c4nc(-c5ccc(-c6ccccc6)c(-c6cccc7ccccc67)c5)nc(-c5c([2H])c([2H])c6c([2H])c([2H])c([2H])c([2H])c6c5[2H])n4)c3c2c1]

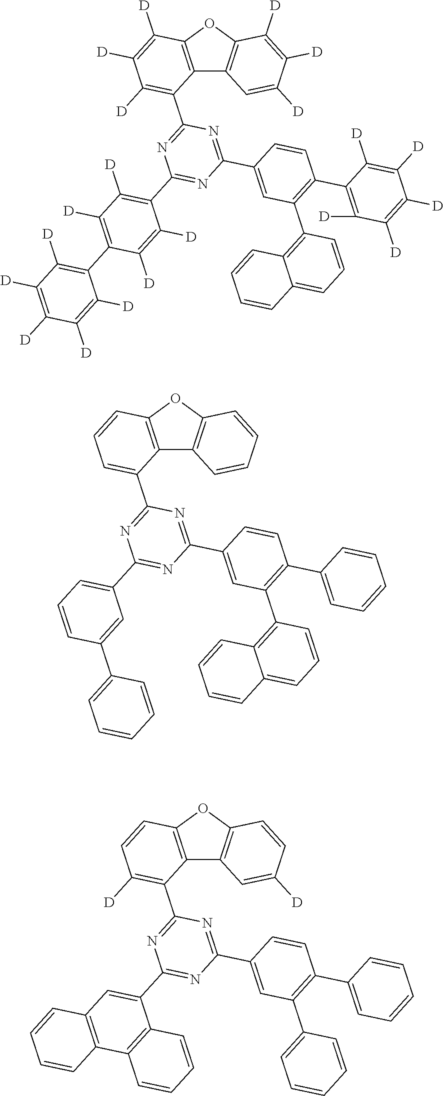 [SMILES: [2H]c1cc2c(oc3c([2H])c([2H])c([2H])c(-c4nc(-c5ccc(-c6c([2H])c([2H])c([2H])c([2H])c6[2H])c(-c6cccc7ccccc67)c5)nc(-c5c([2H])c([2H])c(-c6c([2H])c([2H])c([2H])c([2H])c6[2H])c([2H])c5[2H])n4)c32)c([2H])c1[2H] ; c1ccc(-c2cccc(-c3nc(-c4ccc(-c5ccccc5)c(-c5cccc6ccccc56)c4)nc(-c4cccc5oc6ccccc6c45)n3)c2)cc1 ; [2H]c1ccc2oc3ccc([2H])c(-c4nc(-c5ccc(-c6ccccc6)c(-c6ccccc6)c5)nc(-c5cc6ccccc6c6ccccc56)n4)c3c2c1]

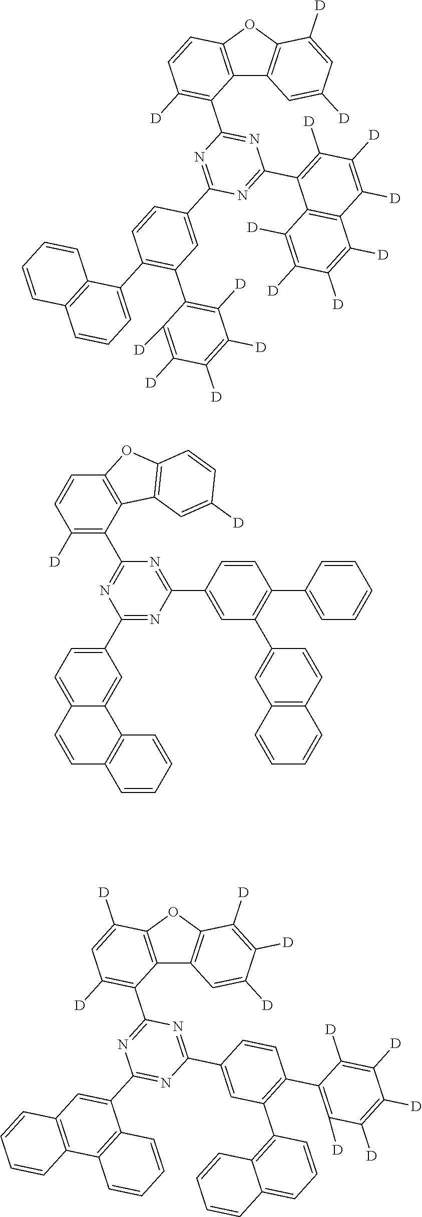 [SMILES: [2H]c1cc([2H])c2oc3ccc([2H])c(-c4nc(-c5ccc(-c6cccc7ccccc67)c(-c6c([2H])c([2H])c([2H])c([2H])c6[2H])c5)nc(-c5c([2H])c([2H])c([2H])c6c([2H])c([2H])c([2H])c([2H])c56)n4)c3c2c1 ; [2H]c1ccc2oc3ccc([2H])c(-c4nc(-c5ccc(-c6ccccc6)c(-c6ccc7ccccc7c6)c5)nc(-c5ccc6ccc7ccccc7c6c5)n4)c3c2c1 ; [2H]c1cc2c(oc3c([2H])cc([2H])c(-c4nc(-c5ccc(-c6c([2H])c([2H])c([2H])c([2H])c6[2H])c(-c6cccc7ccccc67)c5)nc(-c5cc6ccccc6c6ccccc56)n4)c32)c([2H])c1[2H]]

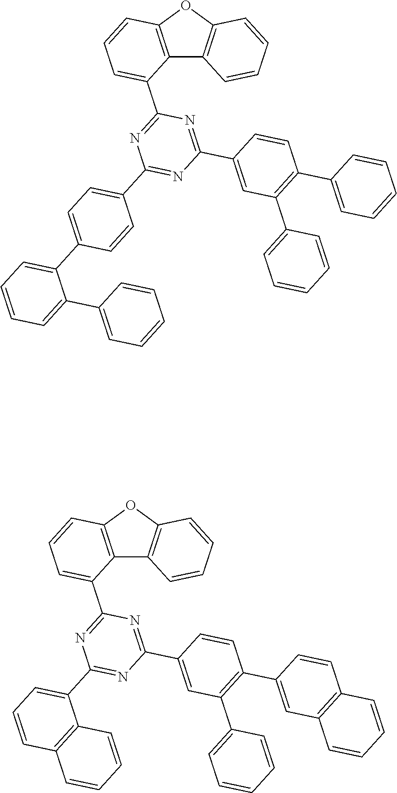 [SMILES: c1ccc(-c2ccccc2-c2ccc(-c3nc(-c4ccc(-c5ccccc5)c(-c5ccccc5)c4)nc(-c4cccc5oc6ccccc6c45)n3)cc2)cc1 ; c1ccc(-c2cc(-c3nc(-c4cccc5ccccc45)nc(-c4cccc5oc6ccccc6c45)n3)ccc2-c2ccc3ccccc3c2)cc1]

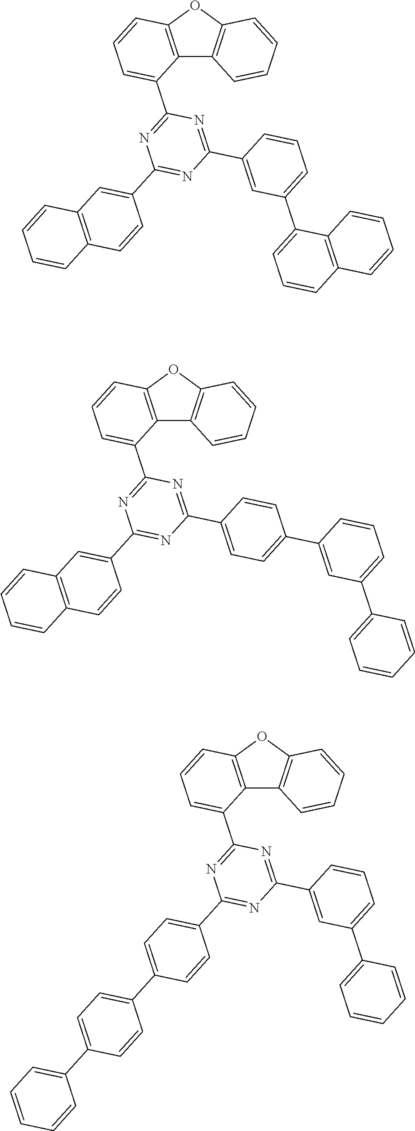 [SMILES: c1cc(-c2nc(-c3ccc4ccccc4c3)nc(-c3cccc4oc5ccccc5c34)n2)cc(-c2cccc3ccccc23)c1 ; c1ccc(-c2cccc(-c3ccc(-c4nc(-c5ccc6ccccc6c5)nc(-c5cccc6oc7ccccc7c56)n4)cc3)c2)cc1 ; c1ccc(-c2ccc(-c3ccc(-c4nc(-c5cccc(-c6ccccc6)c5)nc(-c5cccc6oc7ccccc7c56)n4)cc3)cc2)cc1]

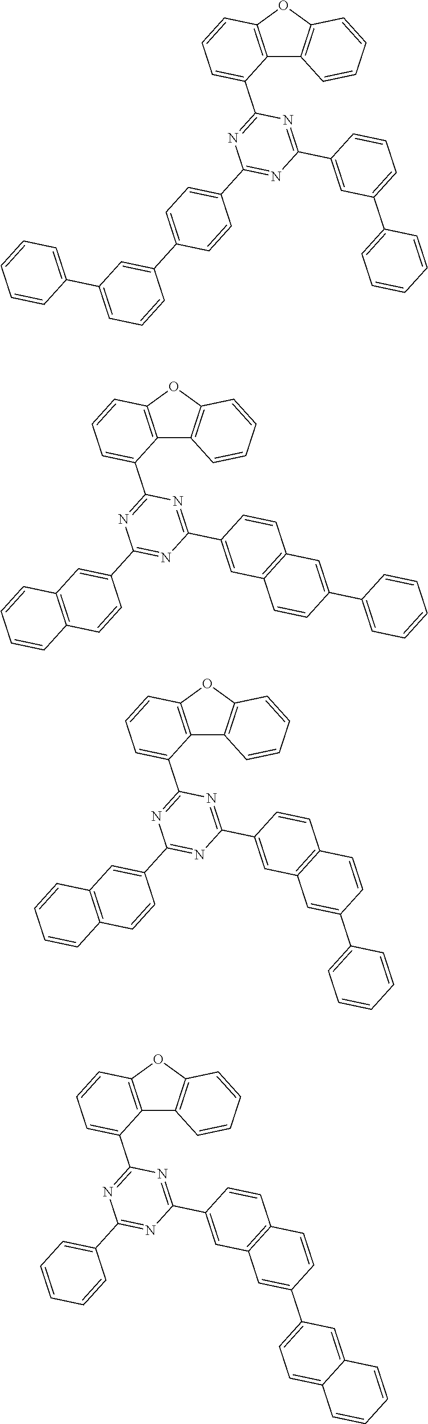 [SMILES: c1ccc(-c2cccc(-c3ccc(-c4nc(-c5cccc(-c6ccccc6)c5)nc(-c5cccc6oc7ccccc7c56)n4)cc3)c2)cc1 ; c1ccc(-c2ccc3cc(-c4nc(-c5ccc6ccccc6c5)nc(-c5cccc6oc7ccccc7c56)n4)ccc3c2)cc1 ; c1ccc(-c2ccc3ccc(-c4nc(-c5ccc6ccccc6c5)nc(-c5cccc6oc7ccccc7c56)n4)cc3c2)cc1 ; c1ccc(-c2nc(-c3ccc4ccc(-c5ccc6ccccc6c5)cc4c3)nc(-c3cccc4oc5ccccc5c34)n2)cc1]

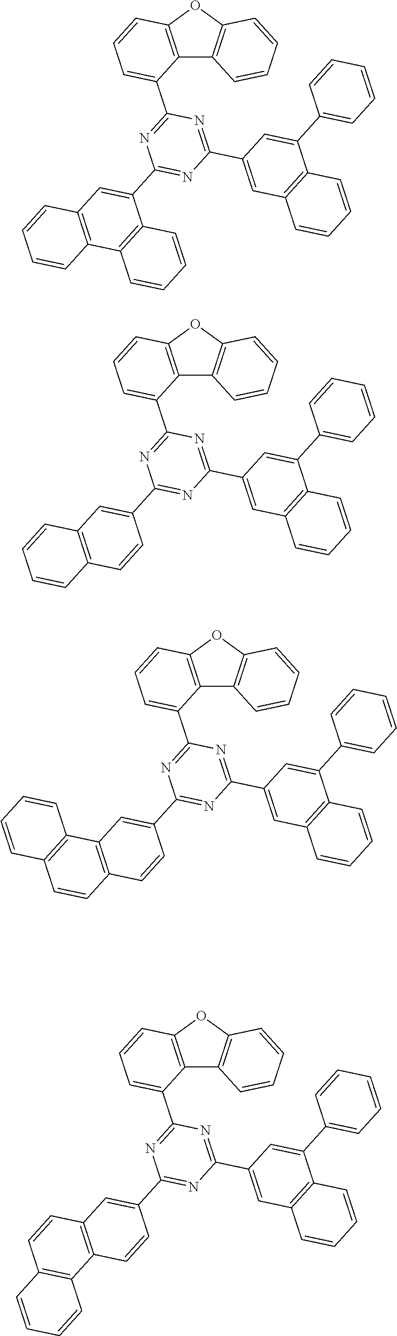 [SMILES: c1ccc(-c2cc(-c3nc(-c4cc5ccccc5c5ccccc45)nc(-c4cccc5oc6ccccc6c45)n3)cc3ccccc23)cc1 ; c1ccc(-c2cc(-c3nc(-c4ccc5ccccc5c4)nc(-c4cccc5oc6ccccc6c45)n3)cc3ccccc23)cc1 ; c1ccc(-c2cc(-c3nc(-c4ccc5ccc6ccccc6c5c4)nc(-c4cccc5oc6ccccc6c45)n3)cc3ccccc23)cc1 ; c1ccc(-c2cc(-c3nc(-c4ccc5c(ccc6ccccc65)c4)nc(-c4cccc5oc6ccccc6c45)n3)cc3ccccc23)cc1]

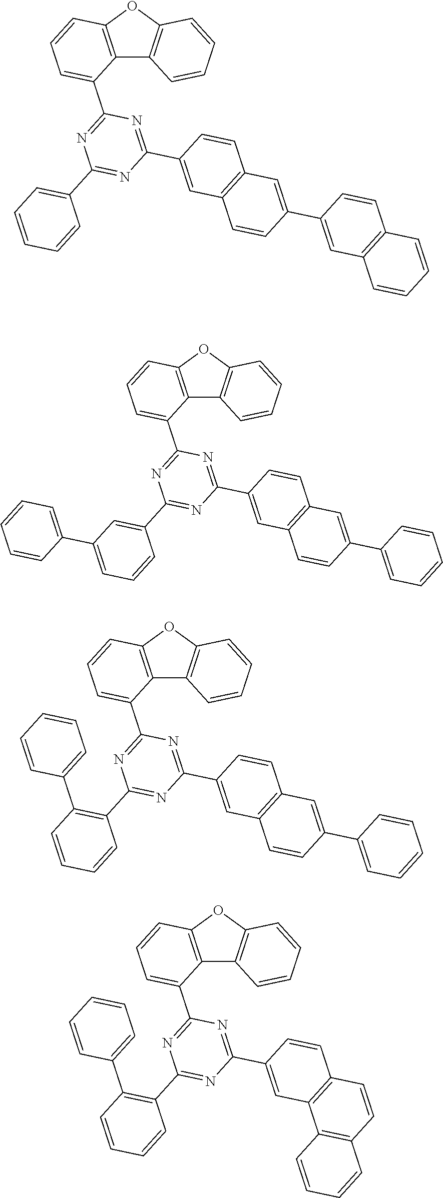 [SMILES: c1ccc(-c2nc(-c3ccc4cc(-c5ccc6ccccc6c5)ccc4c3)nc(-c3cccc4oc5ccccc5c34)n2)cc1 ; c1ccc(-c2cccc(-c3nc(-c4ccc5cc(-c6ccccc6)ccc5c4)nc(-c4cccc5oc6ccccc6c45)n3)c2)cc1 ; c1ccc(-c2ccc3cc(-c4nc(-c5ccccc5-c5ccccc5)nc(-c5cccc6oc7ccccc7c56)n4)ccc3c2)cc1 ; c1ccc(-c2ccccc2-c2nc(-c3ccc4ccc5ccccc5c4c3)nc(-c3cccc4oc5ccccc5c34)n2)cc1]

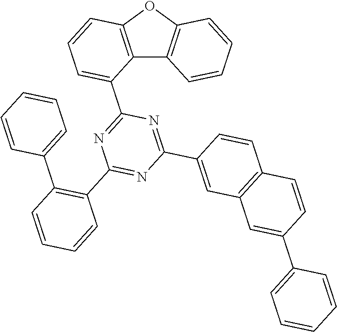 [SMILES: c1ccc(-c2ccc3ccc(-c4nc(-c5ccccc5-c5ccccc5)nc(-c5cccc6oc7ccccc7c56)n4)cc3c2)cc1]

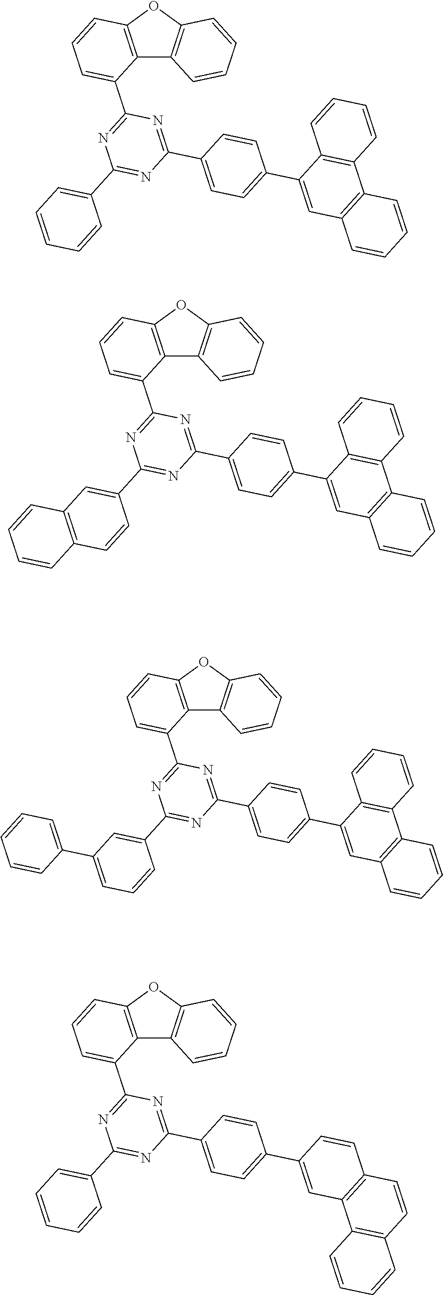 [SMILES: c1ccc(-c2nc(-c3ccc(-c4cc5ccccc5c5ccccc45)cc3)nc(-c3cccc4oc5ccccc5c34)n2)cc1 ; c1ccc2cc(-c3nc(-c4ccc(-c5cc6ccccc6c6ccccc56)cc4)nc(-c4cccc5oc6ccccc6c45)n3)ccc2c1 ; c1ccc(-c2cccc(-c3nc(-c4ccc(-c5cc6ccccc6c6ccccc56)cc4)nc(-c4cccc5oc6ccccc6c45)n3)c2)cc1 ; c1ccc(-c2nc(-c3ccc(-c4ccc5ccc6ccccc6c5c4)cc3)nc(-c3cccc4oc5ccccc5c34)n2)cc1]

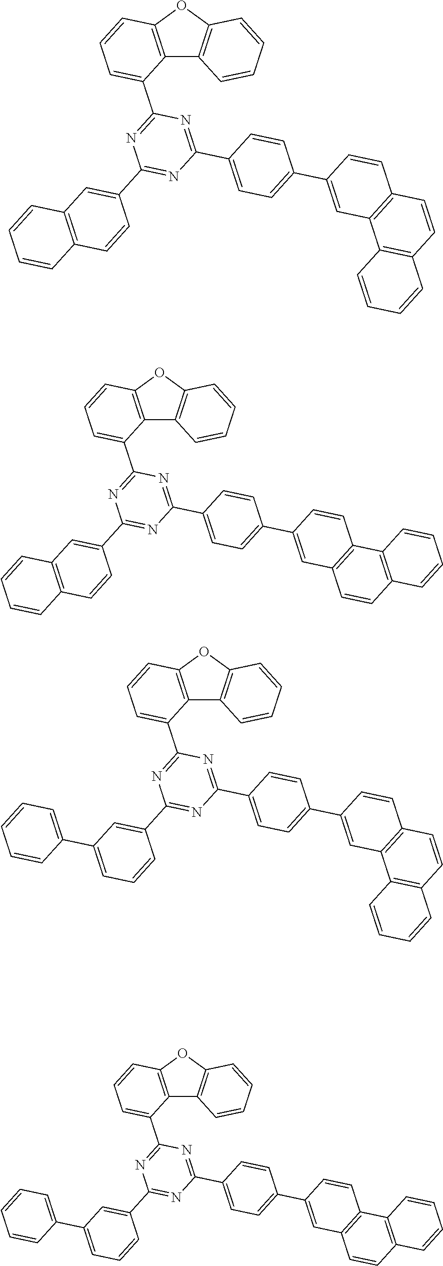 [SMILES: c1ccc2cc(-c3nc(-c4ccc(-c5ccc6ccc7ccccc7c6c5)cc4)nc(-c4cccc5oc6ccccc6c45)n3)ccc2c1 ; c1ccc2cc(-c3nc(-c4ccc(-c5ccc6c(ccc7ccccc76)c5)cc4)nc(-c4cccc5oc6ccccc6c45)n3)ccc2c1 ; c1ccc(-c2cccc(-c3nc(-c4ccc(-c5ccc6ccc7ccccc7c6c5)cc4)nc(-c4cccc5oc6ccccc6c45)n3)c2)cc1 ; c1ccc(-c2cccc(-c3nc(-c4ccc(-c5ccc6c(ccc7ccccc76)c5)cc4)nc(-c4cccc5oc6ccccc6c45)n3)c2)cc1]

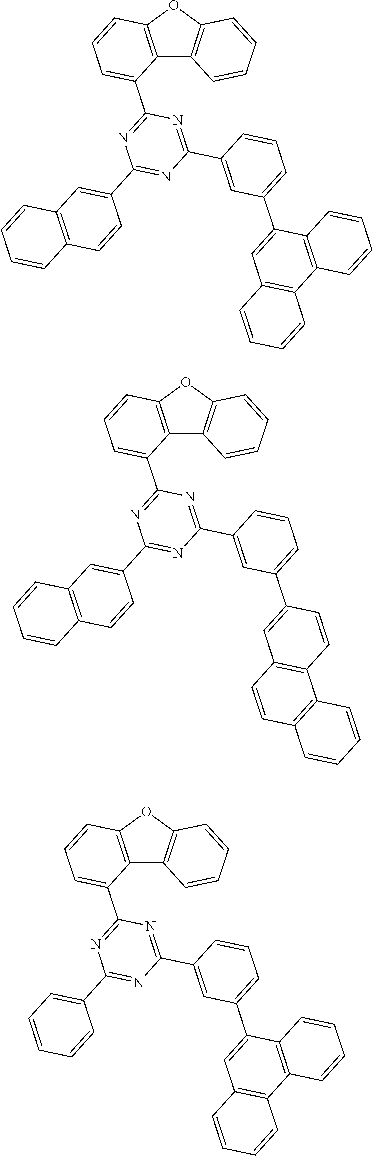 [SMILES: c1cc(-c2nc(-c3ccc4ccccc4c3)nc(-c3cccc4oc5ccccc5c34)n2)cc(-c2cc3ccccc3c3ccccc23)c1 ; c1cc(-c2ccc3c(ccc4ccccc43)c2)cc(-c2nc(-c3ccc4ccccc4c3)nc(-c3cccc4oc5ccccc5c34)n2)c1 ; c1ccc(-c2nc(-c3cccc(-c4cc5ccccc5c5ccccc45)c3)nc(-c3cccc4oc5ccccc5c34)n2)cc1]

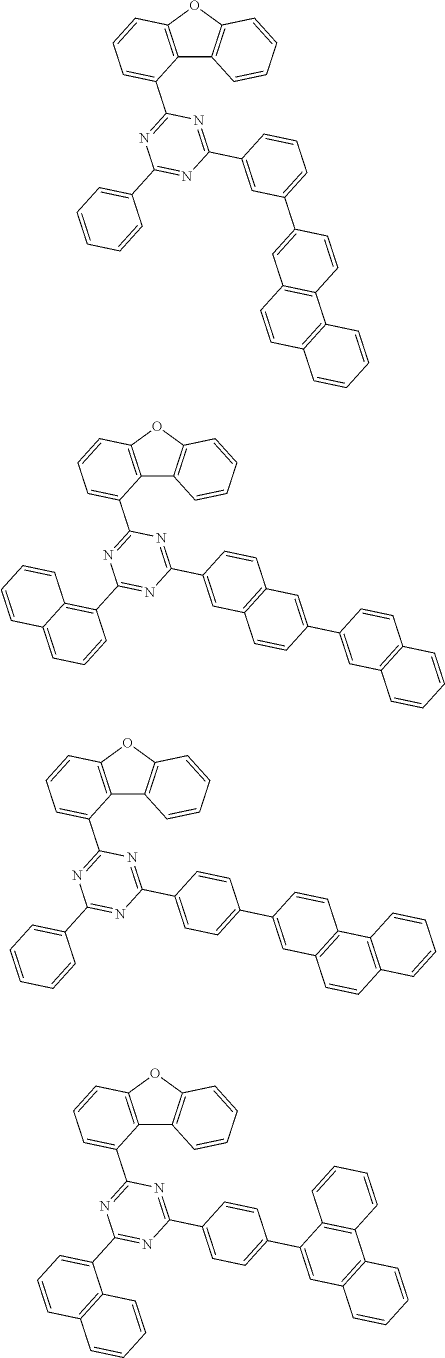 [SMILES: c1ccc(-c2nc(-c3cccc(-c4ccc5c(ccc6ccccc65)c4)c3)nc(-c3cccc4oc5ccccc5c34)n2)cc1 ; c1ccc2cc(-c3ccc4cc(-c5nc(-c6cccc7ccccc67)nc(-c6cccc7oc8ccccc8c67)n5)ccc4c3)ccc2c1 ; c1ccc(-c2nc(-c3ccc(-c4ccc5c(ccc6ccccc65)c4)cc3)nc(-c3cccc4oc5ccccc5c34)n2)cc1 ; c1ccc2c(-c3nc(-c4ccc(-c5cc6ccccc6c6ccccc56)cc4)nc(-c4cccc5oc6ccccc6c45)n3)cccc2c1]

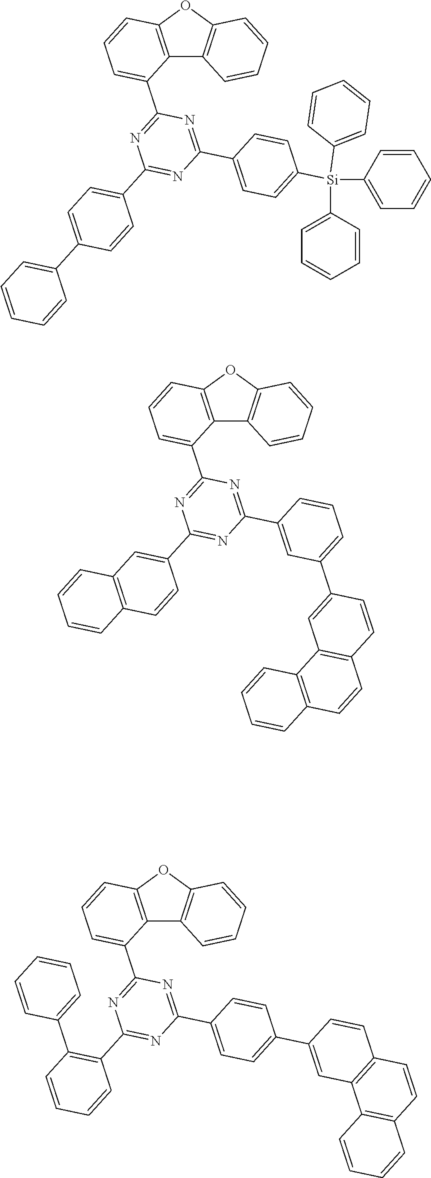 [SMILES: c1ccc(-c2ccc(-c3nc(-c4ccc([Si](c5ccccc5)(c5ccccc5)c5ccccc5)cc4)nc(-c4cccc5oc6ccccc6c45)n3)cc2)cc1 ; c1cc(-c2ccc3ccc4ccccc4c3c2)cc(-c2nc(-c3ccc4ccccc4c3)nc(-c3cccc4oc5ccccc5c34)n2)c1 ; c1ccc(-c2ccccc2-c2nc(-c3ccc(-c4ccc5ccc6ccccc6c5c4)cc3)nc(-c3cccc4oc5ccccc5c34)n2)cc1]

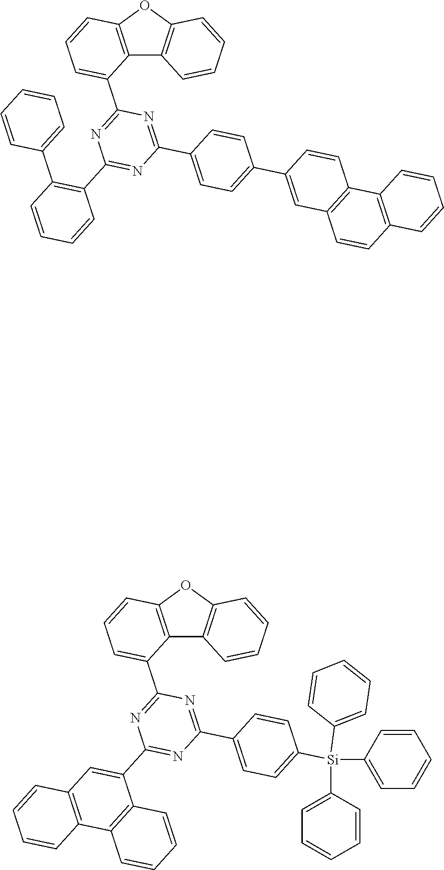 [SMILES: c1ccc(-c2ccccc2-c2nc(-c3ccc(-c4ccc5c(ccc6ccccc65)c4)cc3)nc(-c3cccc4oc5ccccc5c34)n2)cc1 ; c1ccc([Si](c2ccccc2)(c2ccccc2)c2ccc(-c3nc(-c4cc5ccccc5c5ccccc45)nc(-c4cccc5oc6ccccc6c45)n3)cc2)cc1]

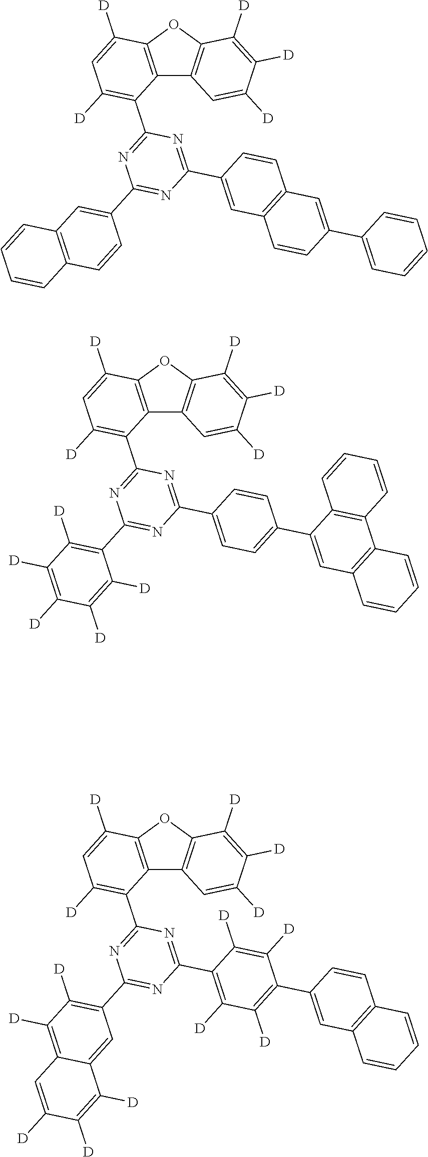 [SMILES: [2H]c1cc2c(oc3c([2H])cc([2H])c(-c4nc(-c5ccc6ccccc6c5)nc(-c5ccc6cc(-c7ccccc7)ccc6c5)n4)c32)c([2H])c1[2H] ; [2H]c1cc2c(oc3c([2H])cc([2H])c(-c4nc(-c5ccc(-c6cc7ccccc7c7ccccc67)cc5)nc(-c5c([2H])c([2H])c([2H])c([2H])c5[2H])n4)c32)c([2H])c1[2H] ; [2H]c1cc2c([2H])c([2H])c(-c3nc(-c4c([2H])c([2H])c(-c5ccc6ccccc6c5)c([2H])c4[2H])nc(-c4c([2H])cc([2H])c5oc6c([2H])c([2H])c([2H])cc6c45)n3)cc2c([2H])c1[2H]]

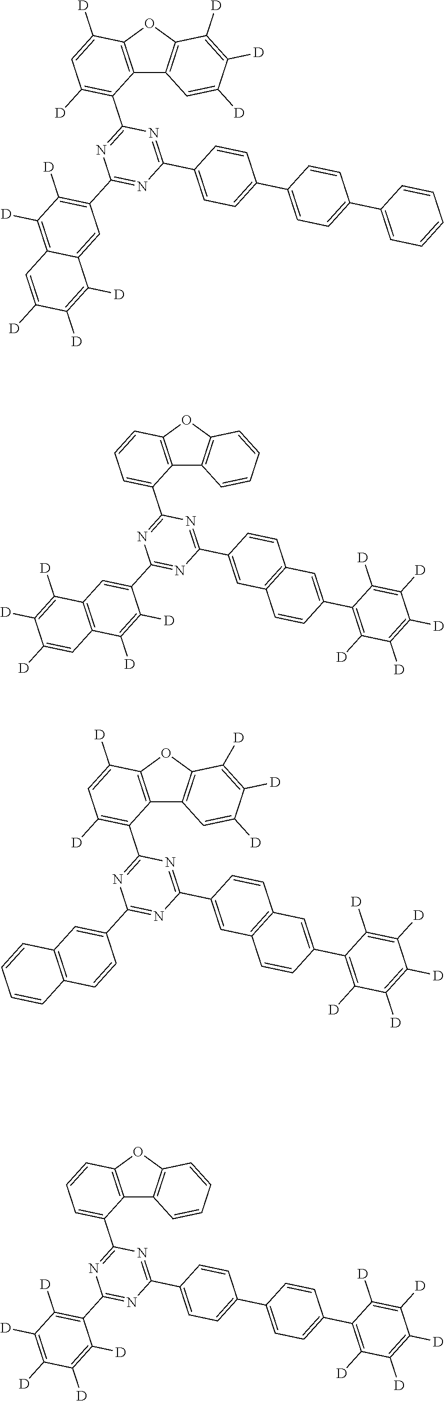

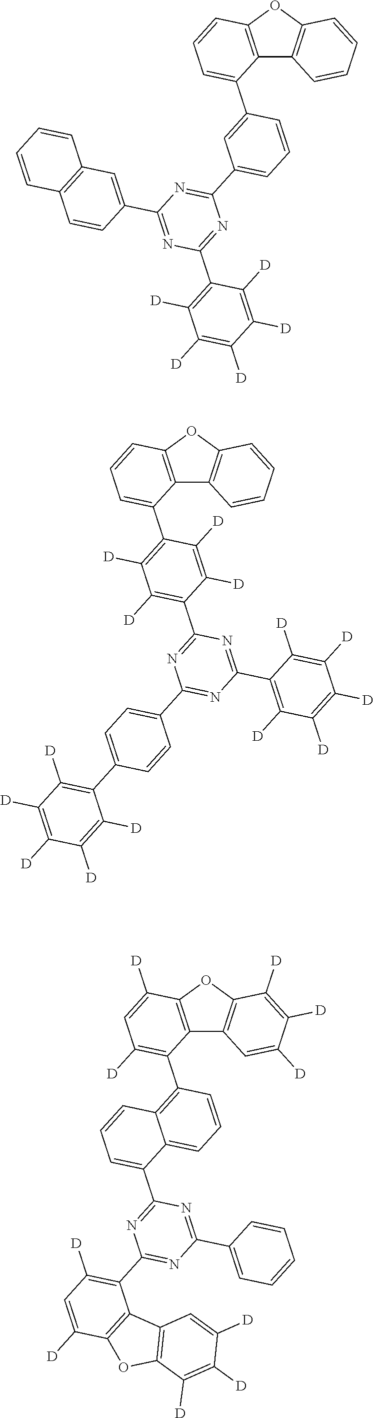 [SMILES: [2H]c1c([2H])c([2H])c(-c2nc(-c3cccc(-c4cccc5oc6ccccc6c45)c3)nc(-c3ccc4ccccc4c3)n2)c([2H])c1[2H] ; [2H]c1c([2H])c([2H])c(-c2ccc(-c3nc(-c4c([2H])c([2H])c([2H])c([2H])c4[2H])nc(-c4c([2H])c([2H])c(-c5cccc6oc7ccccc7c56)c([2H])c4[2H])n3)cc2)c([2H])c1[2H] ; [2H]c1cc2c(oc3c([2H])cc([2H])c(-c4nc(-c5ccccc5)nc(-c5cccc6c(-c7c([2H])cc([2H])c8oc9c([2H])c([2H])c([2H])cc9c78)cccc56)n4)c32)c([2H])c1[2H]]

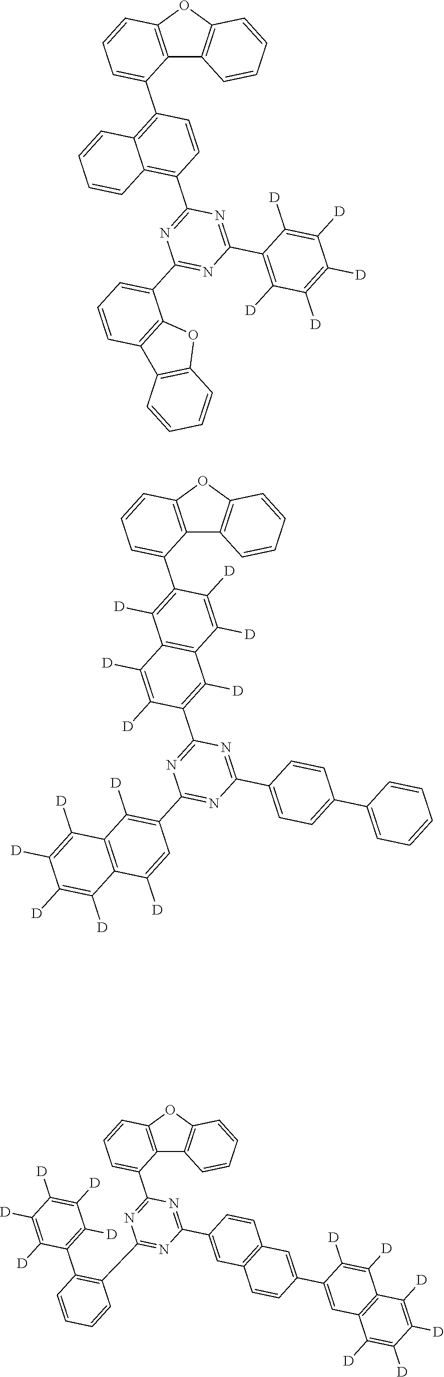 [SMILES: [2H]c1c([2H])c([2H])c(-c2nc(-c3ccc(-c4cccc5oc6ccccc6c45)c4ccccc34)nc(-c3cccc4c3oc3ccccc34)n2)c([2H])c1[2H] ; [2H]c1c([2H])c([2H])c2c([2H])c(-c3nc(-c4ccc(-c5ccccc5)cc4)nc(-c4c([2H])c([2H])c5c([2H])c(-c6cccc7oc8ccccc8c67)c([2H])c([2H])c5c4[2H])n3)cc([2H])c2c1[2H] ; [2H]c1c([2H])c([2H])c(-c2ccccc2-c2nc(-c3ccc4cc(-c5cc6c([2H])c([2H])c([2H])c([2H])c6c([2H])c5[2H])ccc4c3)nc(-c3cccc4oc5ccccc5c34)n2)c([2H])c1[2H]]

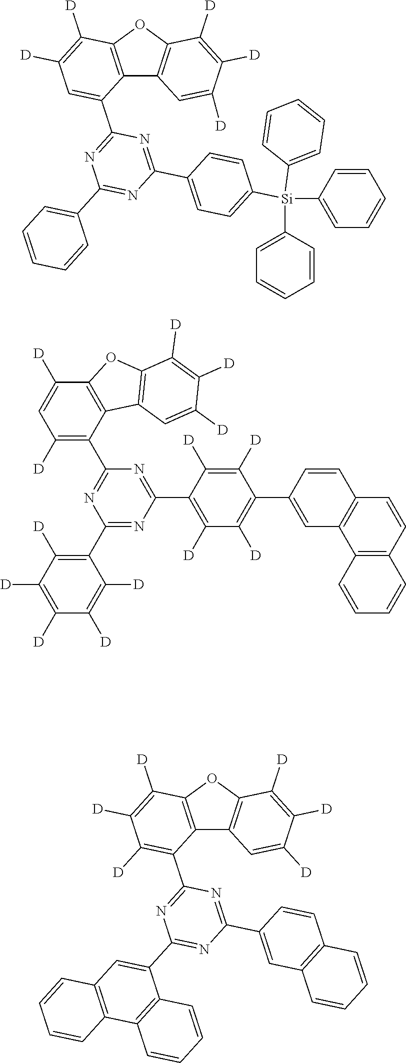 [SMILES: [2H]c1cc2c(oc3c([2H])c([2H])cc(-c4nc(-c5ccccc5)nc(-c5ccc([Si](c6ccccc6)(c6ccccc6)c6ccccc6)cc5)n4)c32)c([2H])c1[2H] ; [2H]c1cc2c(oc3c([2H])cc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c32)c([2H])c1[2H] ; [2H]c1cc2c(oc3c([2H])c([2H])c([2H])c(-c4nc(-c5ccc6ccccc6c5)nc(-c5cc6ccccc6c6ccccc56)n4)c32)c([2H])c1[2H]]

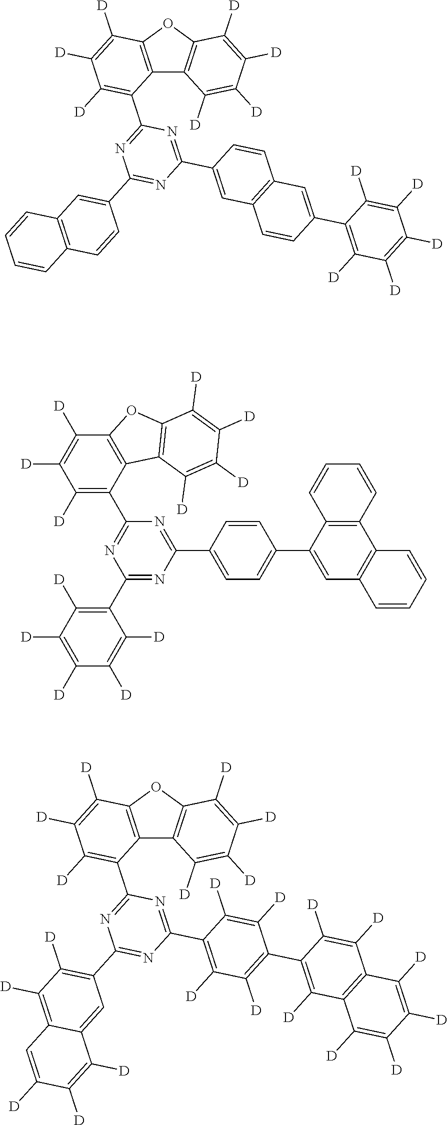 [SMILES: [2H]c1c([2H])c([2H])c(-c2ccc3cc(-c4nc(-c5ccc6ccccc6c5)nc(-c5c([2H])c([2H])c([2H])c6oc7c([2H])c([2H])c([2H])c([2H])c7c56)n4)ccc3c2)c([2H])c1[2H] ; [2H]c1c([2H])c([2H])c(-c2nc(-c3ccc(-c4cc5ccccc5c5ccccc45)cc3)nc(-c3c([2H])c([2H])c([2H])c4oc5c([2H])c([2H])c([2H])c([2H])c5c34)n2)c([2H])c1[2H] ; [2H]c1cc2c([2H])c([2H])c(-c3nc(-c4c([2H])c([2H])c(-c5c([2H])c([2H])c6c([2H])c([2H])c([2H])c([2H])c6c5[2H])c([2H])c4[2H])nc(-c4c([2H])c([2H])c([2H])c5oc6c([2H])c([2H])c([2H])c([2H])c6c45)n3)cc2c([2H])c1[2H]]

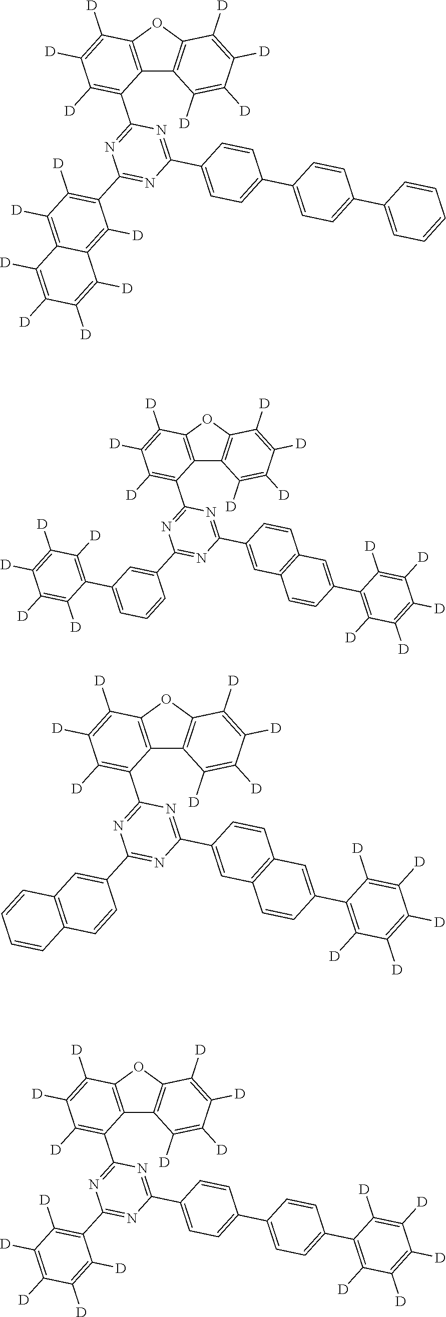

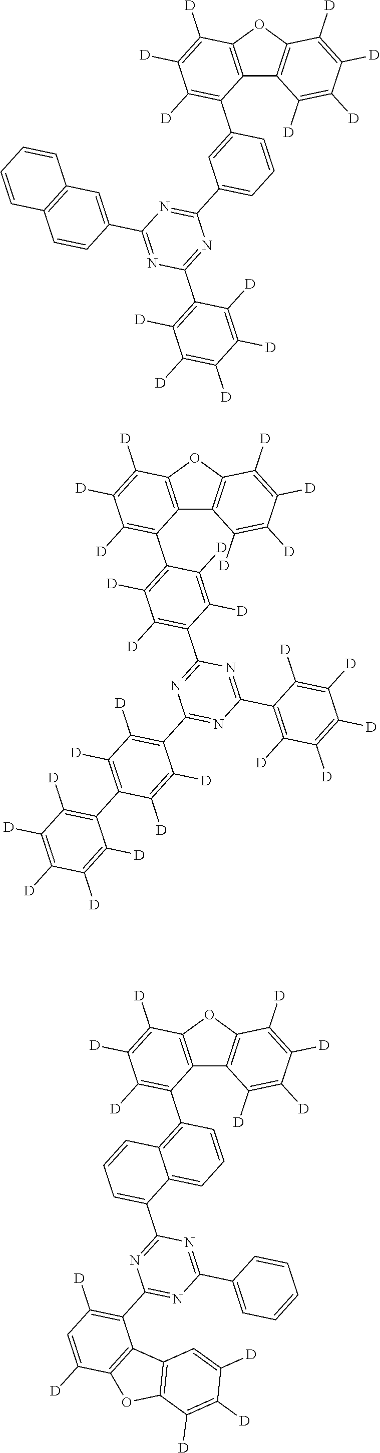

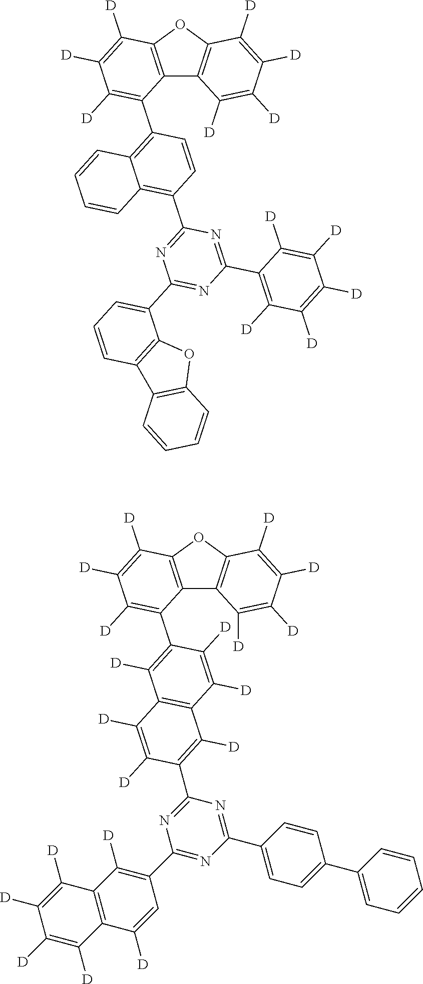 [SMILES: [2H]c1c([2H])c([2H])c(-c2nc(-c3ccc(-c4c([2H])c([2H])c([2H])c5oc6c([2H])c([2H])c([2H])c([2H])c6c45)c4ccccc34)nc(-c3cccc4c3oc3ccccc34)n2)c([2H])c1[2H] ; [2H]c1c([2H])c([2H])c2c(oc3c([2H])c([2H])c([2H])c(-c4c([2H])c([2H])c5c([2H])c(-c6nc(-c7ccc(-c8ccccc8)cc7)nc(-c7cc([2H])c8c([2H])c([2H])c([2H])c([2H])c8c7[2H])n6)c([2H])c([2H])c5c4[2H])c32)c1[2H]]

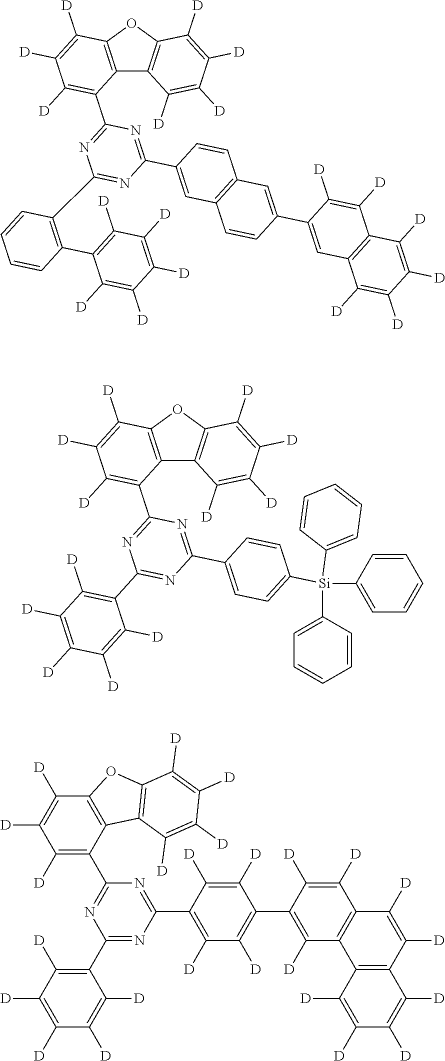

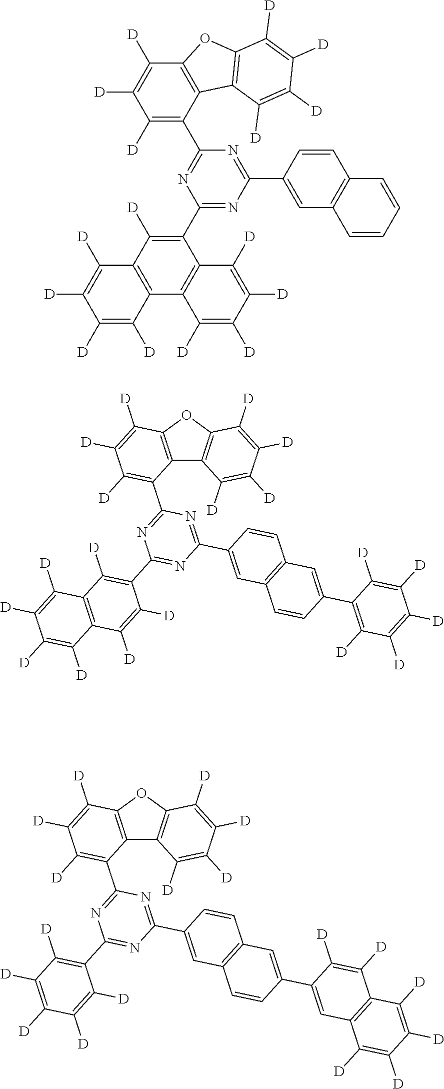

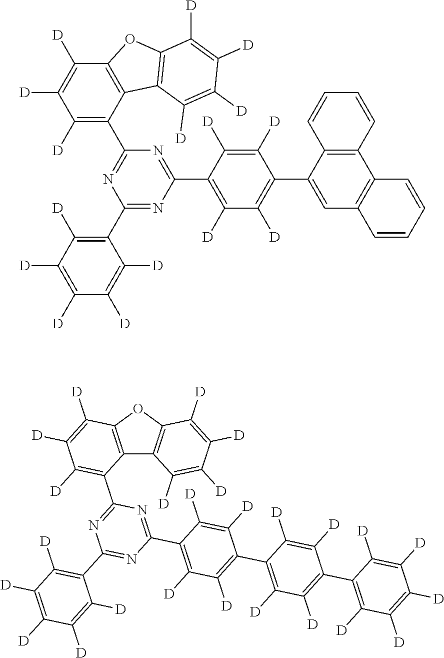 [SMILES: [2H]c1c([2H])c([2H])c(-c2nc(-c3c([2H])c([2H])c(-c4cc5ccccc5c5ccccc45)c([2H])c3[2H])nc(-c3c([2H])c([2H])c([2H])c4oc5c([2H])c([2H])c([2H])c([2H])c5c34)n2)c([2H])c1[2H] ; [2H]c1c([2H])c([2H])c(-c2nc(-c3c([2H])c([2H])c(-c4c([2H])c([2H])c(-c5c([2H])c([2H])c([2H])c([2H])c5[2H])c([2H])c4[2H])c([2H])c3[2H])nc(-c3c([2H])c([2H])c([2H])c4oc5c([2H])c([2H])c([2H])c([2H])c5c34)n2)c([2H])c1[2H]]

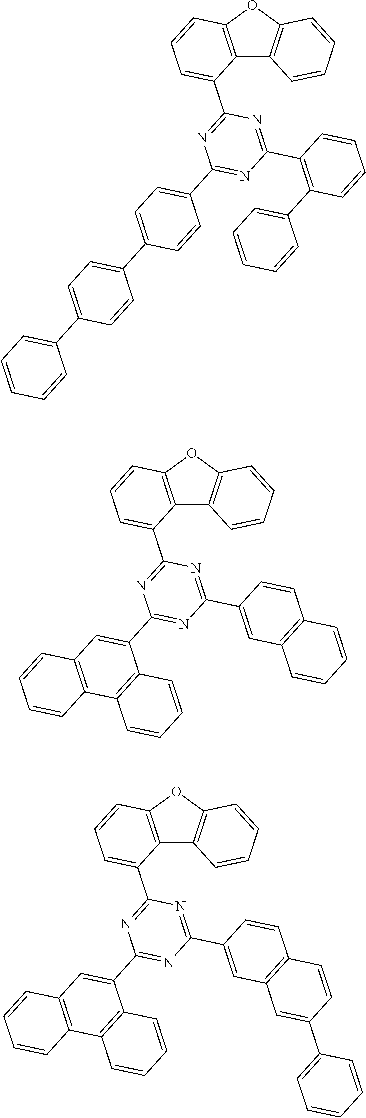 [SMILES: c1ccc(-c2ccc(-c3ccc(-c4nc(-c5ccccc5-c5ccccc5)nc(-c5cccc6oc7ccccc7c56)n4)cc3)cc2)cc1 ; c1ccc2cc(-c3nc(-c4cc5ccccc5c5ccccc45)nc(-c4cccc5oc6ccccc6c45)n3)ccc2c1 ; c1ccc(-c2ccc3ccc(-c4nc(-c5cc6ccccc6c6ccccc56)nc(-c5cccc6oc7ccccc7c56)n4)cc3c2)cc1]

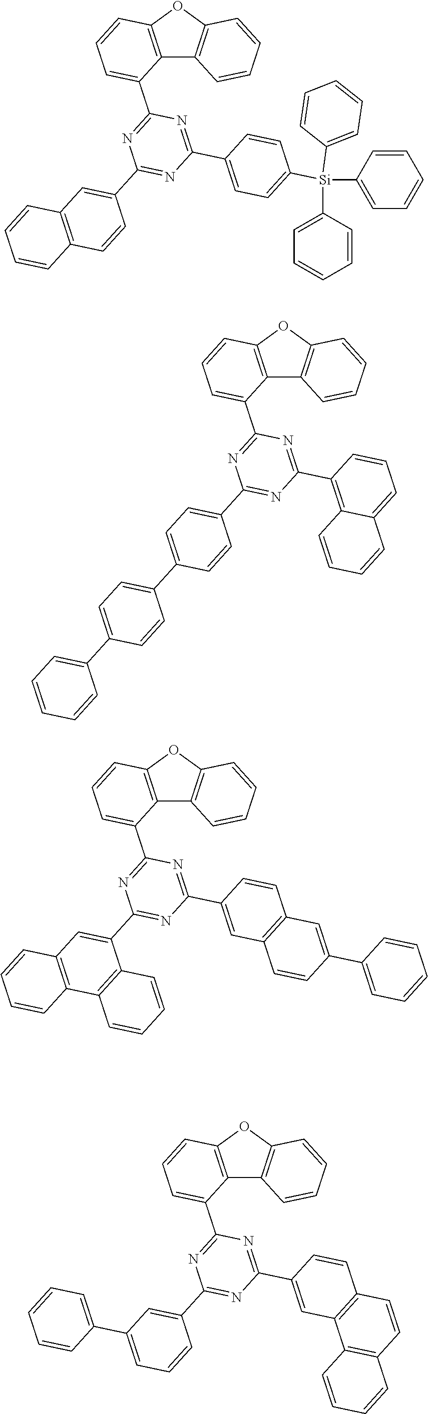 [SMILES: c1ccc([Si](c2ccccc2)(c2ccccc2)c2ccc(-c3nc(-c4ccc5ccccc5c4)nc(-c4cccc5oc6ccccc6c45)n3)cc2)cc1 ; c1ccc(-c2ccc(-c3ccc(-c4nc(-c5cccc6ccccc56)nc(-c5cccc6oc7ccccc7c56)n4)cc3)cc2)cc1 ; c1ccc(-c2ccc3cc(-c4nc(-c5cc6ccccc6c6ccccc56)nc(-c5cccc6oc7ccccc7c56)n4)ccc3c2)cc1 ; c1ccc(-c2cccc(-c3nc(-c4ccc5ccc6ccccc6c5c4)nc(-c4cccc5oc6ccccc6c45)n3)c2)cc1]

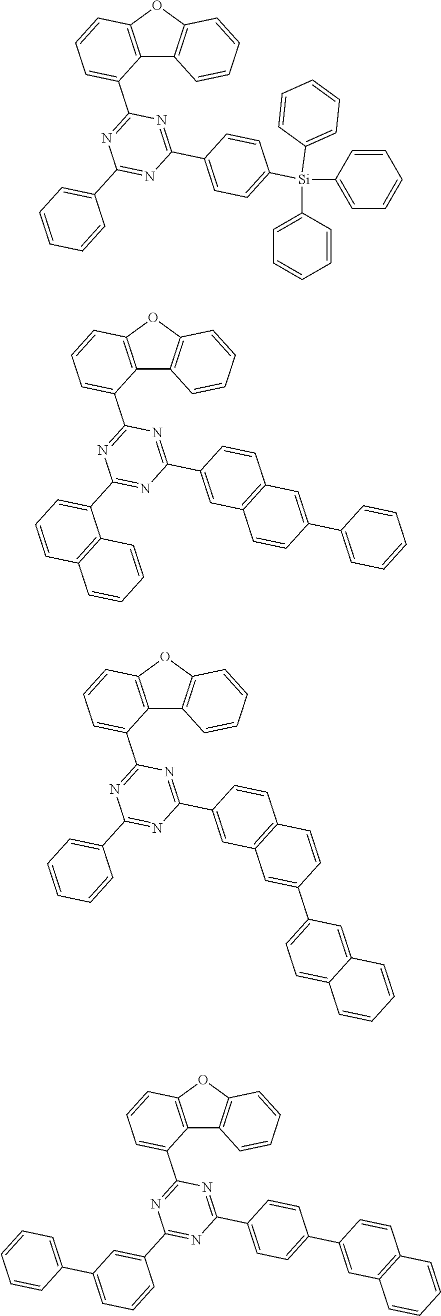 [SMILES: c1ccc(-c2nc(-c3ccc([Si](c4ccccc4)(c4ccccc4)c4ccccc4)cc3)nc(-c3cccc4oc5ccccc5c34)n2)cc1 ; c1ccc(-c2ccc3cc(-c4nc(-c5cccc6ccccc56)nc(-c5cccc6oc7ccccc7c56)n4)ccc3c2)cc1 ; c1ccc(-c2nc(-c3ccc4ccc(-c5ccc6ccccc6c5)cc4c3)nc(-c3cccc4oc5ccccc5c34)n2)cc1 ; c1ccc(-c2cccc(-c3nc(-c4ccc(-c5ccc6ccccc6c5)cc4)nc(-c4cccc5oc6ccccc6c45)n3)c2)cc1]

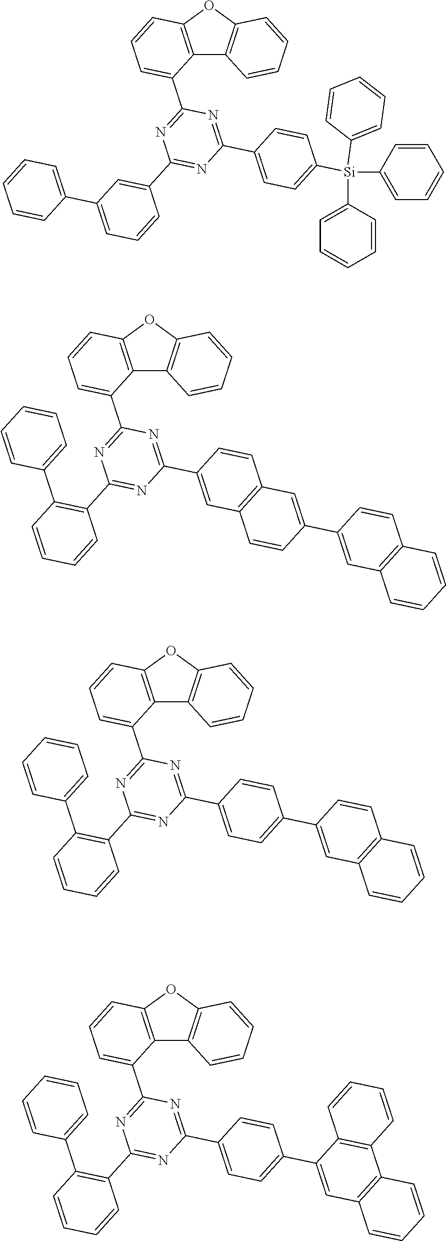 [SMILES: c1ccc(-c2cccc(-c3nc(-c4ccc([Si](c5ccccc5)(c5ccccc5)c5ccccc5)cc4)nc(-c4cccc5oc6ccccc6c45)n3)c2)cc1 ; c1ccc(-c2ccccc2-c2nc(-c3ccc4cc(-c5ccc6ccccc6c5)ccc4c3)nc(-c3cccc4oc5ccccc5c34)n2)cc1 ; c1ccc(-c2ccccc2-c2nc(-c3ccc(-c4ccc5ccccc5c4)cc3)nc(-c3cccc4oc5ccccc5c34)n2)cc1 ; c1ccc(-c2ccccc2-c2nc(-c3ccc(-c4cc5ccccc5c5ccccc45)cc3)nc(-c3cccc4oc5ccccc5c34)n2)cc1]

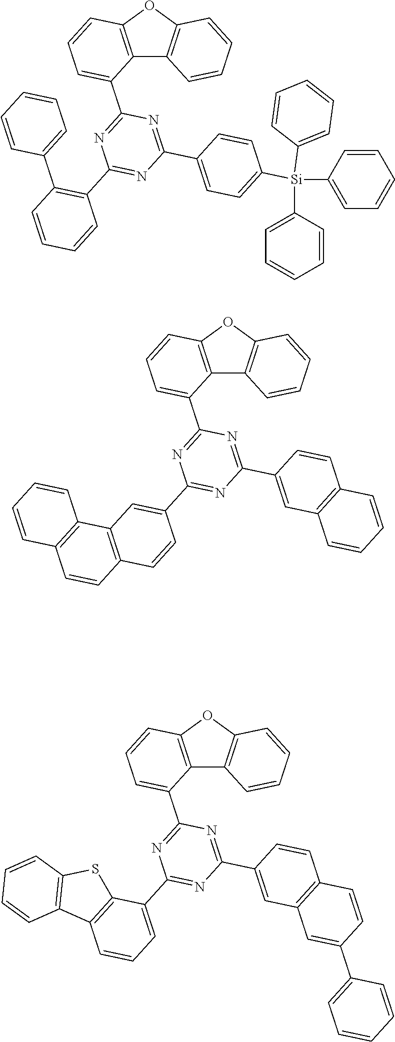 [SMILES: c1ccc(-c2ccccc2-c2nc(-c3ccc([Si](c4ccccc4)(c4ccccc4)c4ccccc4)cc3)nc(-c3cccc4oc5ccccc5c34)n2)cc1 ; c1ccc2cc(-c3nc(-c4ccc5ccc6ccccc6c5c4)nc(-c4cccc5oc6ccccc6c45)n3)ccc2c1 ; c1ccc(-c2ccc3ccc(-c4nc(-c5cccc6c5sc5ccccc56)nc(-c5cccc6oc7ccccc7c56)n4)cc3c2)cc1]

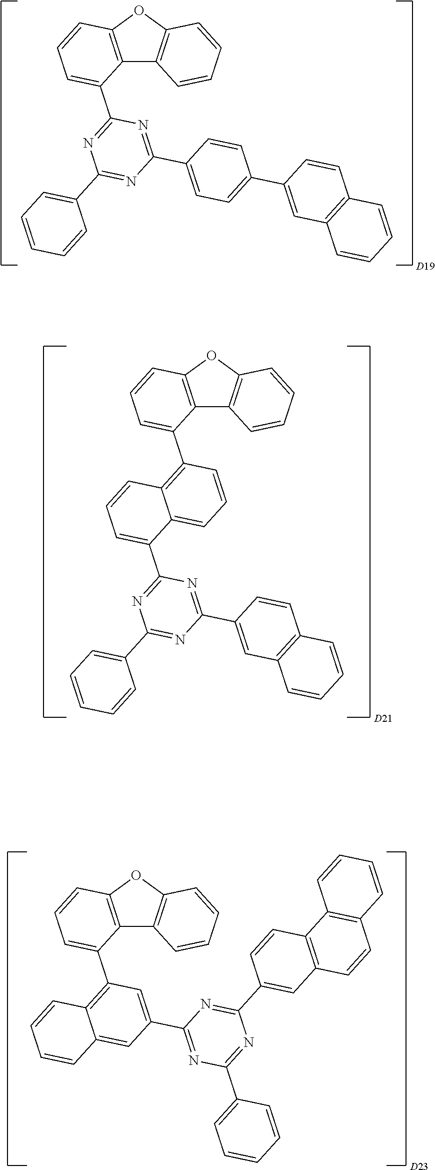

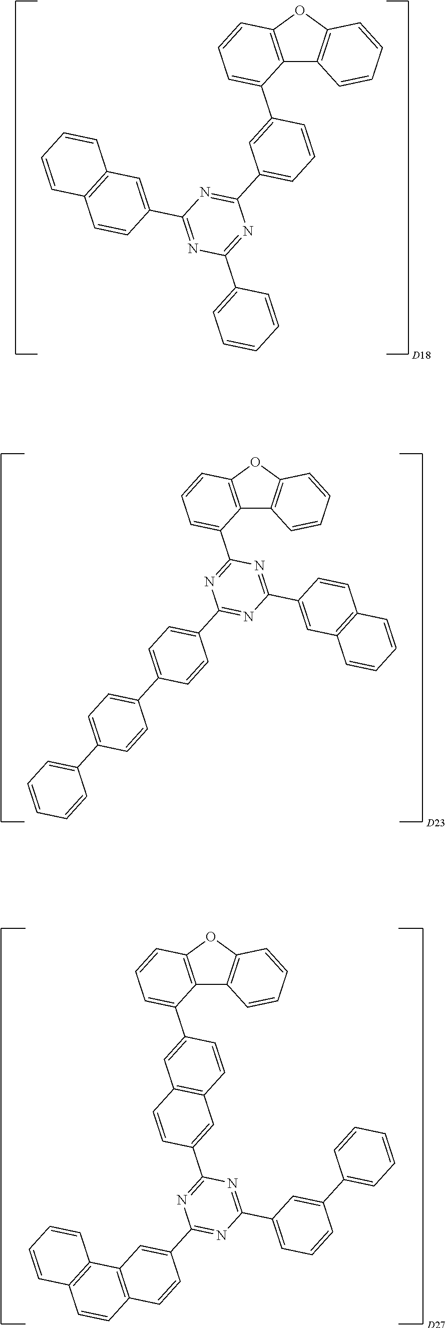

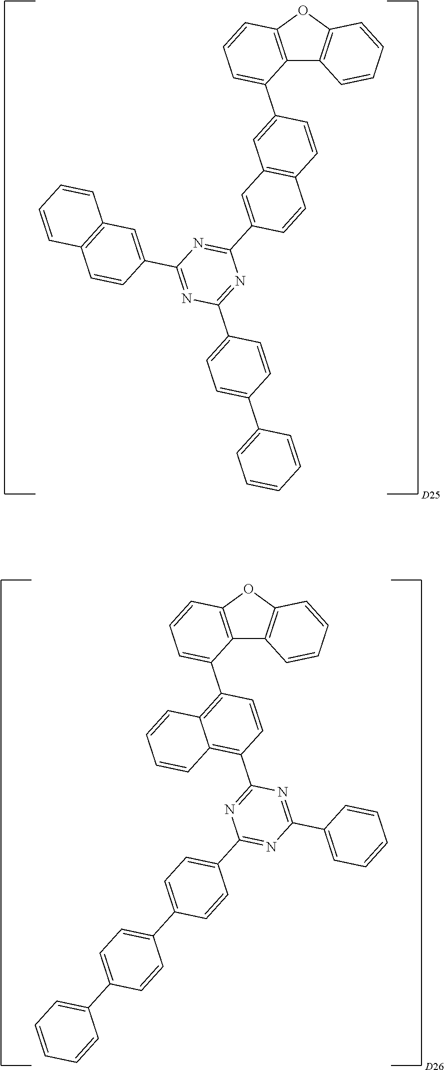

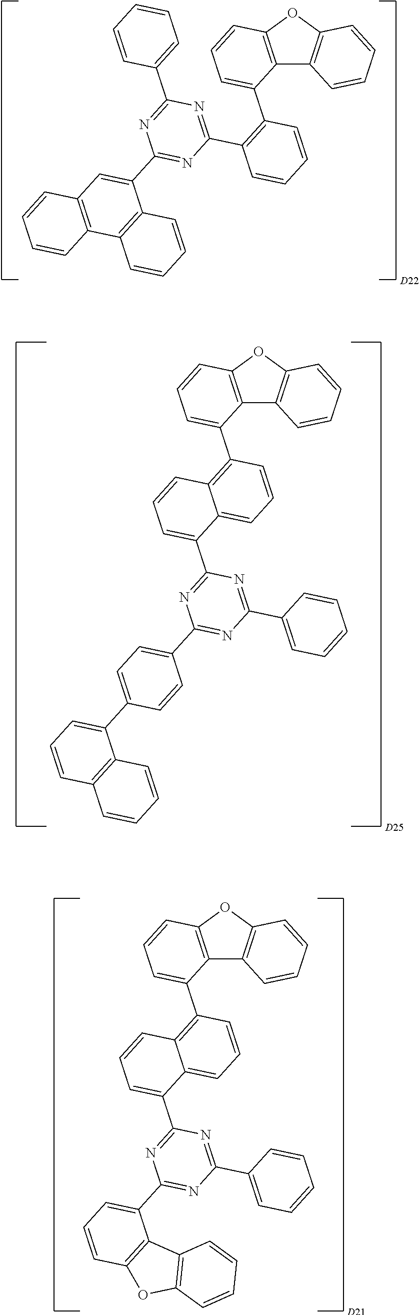

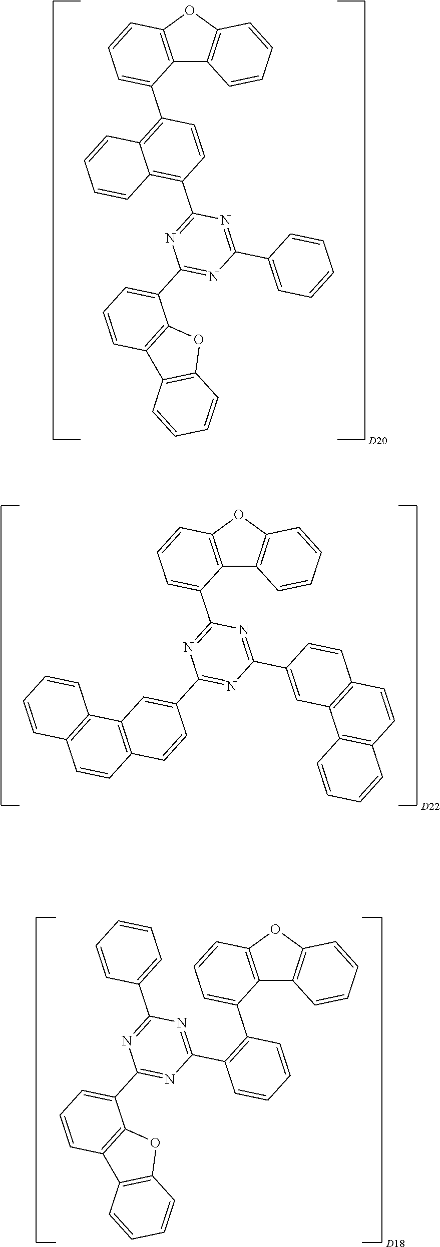

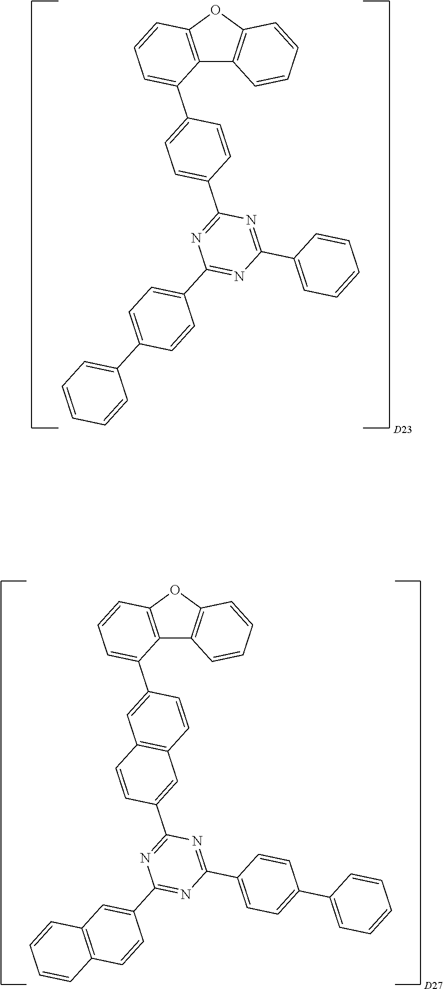

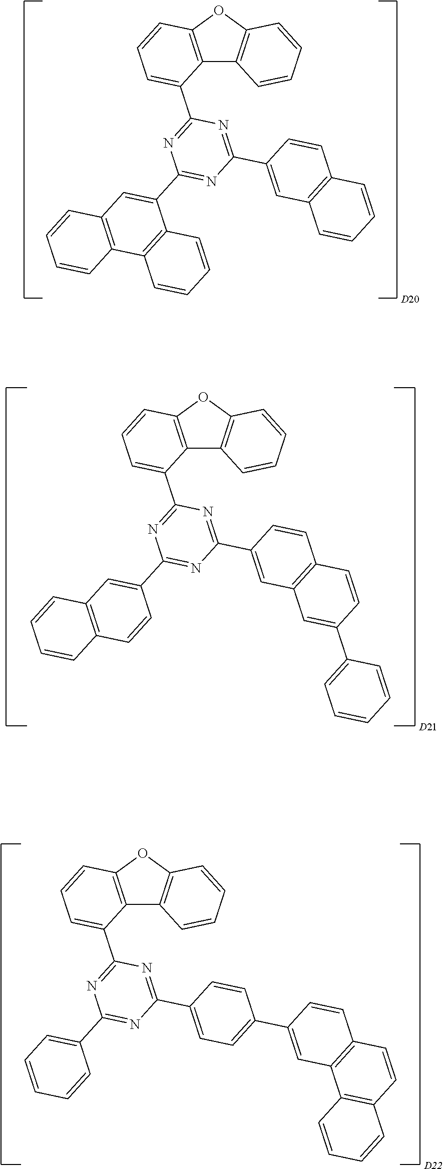

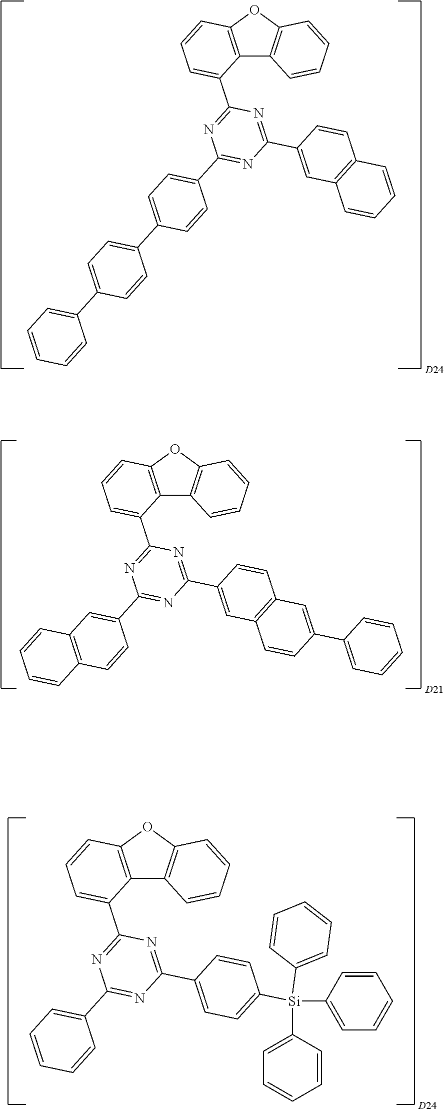

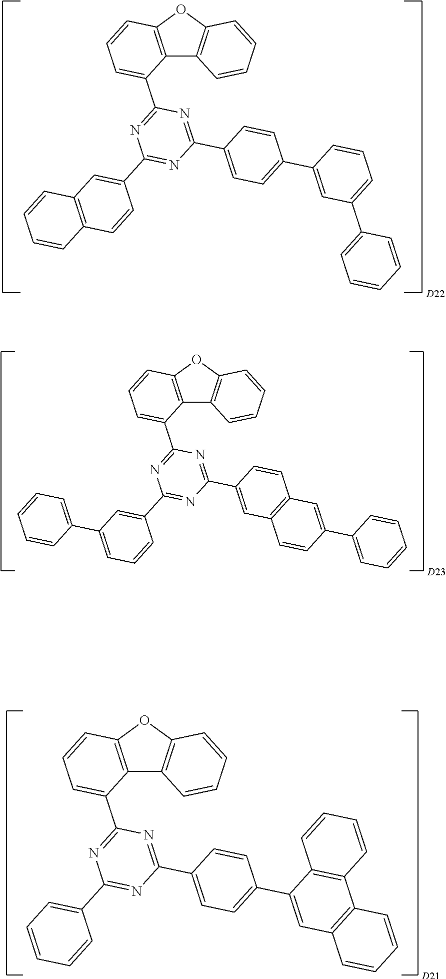

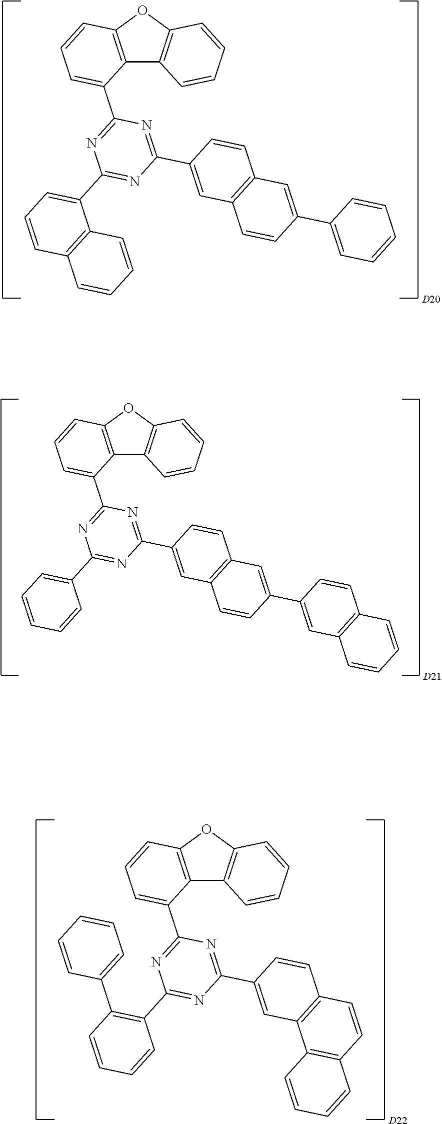

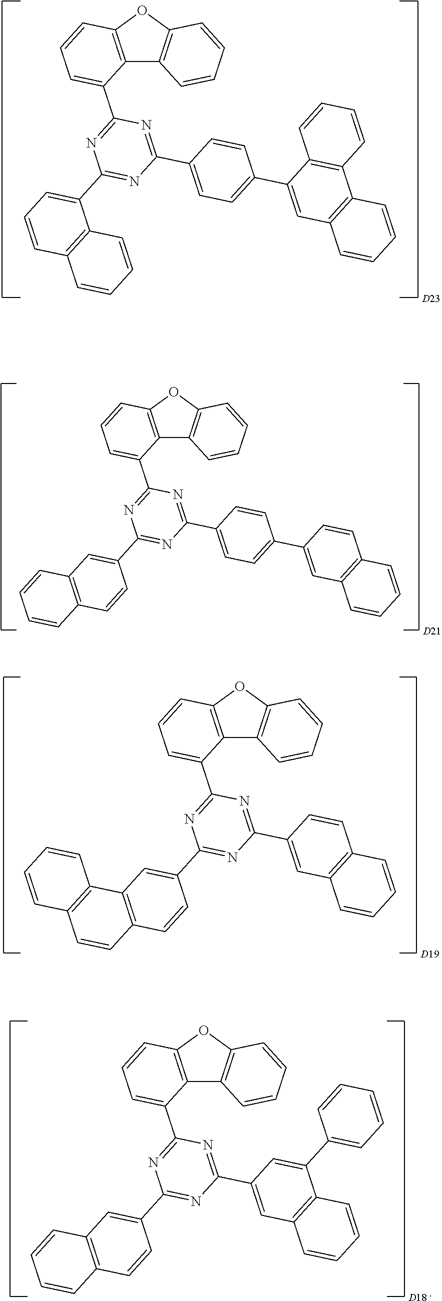

The compound of Chemical Formula 1 can be prepared by a preparation method as shown in the following Reaction Scheme 1 as an example, and other remaining compounds can be prepared in a similar manner.

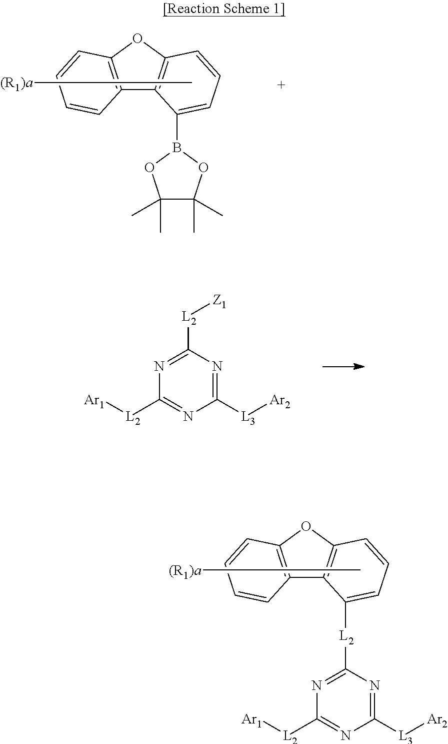

in Reaction Scheme 1, Ar₁ and Are, L₁ to L₃, R₁ and a are as defined in Chemical Formula 2, and Z₁ is halogen, preferably Z₁ is chloro or bromo.

Reaction Scheme 1 is a Suzuki coupling reaction, which is preferably carried out in the presence of a palladium catalyst and a base, and a reactive group for the Suzuki coupling reaction can be modified as known in the art. The above preparation method can be further embodied in Preparation Examples described hereinafter.

Preferably, the compound of Chemical Formula 2 can be any one of the following Chemical Formula 2-1 and Chemical Formula 2-2:

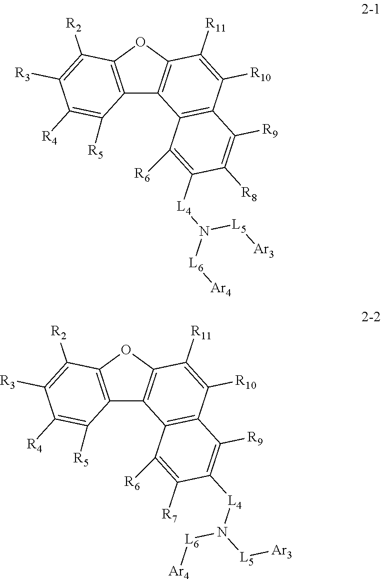

in Chemical Formula 2-1 and Chemical Formula 2-2,
R₂ to R₁₁, Ar₃, Ar₄ and L₄ to L₆ are as defined in Chemical Formula 2.

Preferably, Ar₃ and Ar₄ are each independently a substituted or unsubstituted C₆₋₂₀ aryl, or a substituted or unsubstituted C₂₋₂₀ heteroaryl containing at least one selected from the group consisting of N, O and S.

More preferably, Ar₃ and Ar₄ can be each independently phenyl, triphenylsilyl phenyl, biphenylyl, terphenylyl, naphthyl, phenyl naphthyl, phenanthrenyl, dibenzofuranyl, dibenzothiophenyl, phenyl carbazolyl, or dimethylfluorenyl, and the hydrogens of Ar₃ and Ar₄ can be each independently unsubstituted or substituted with deuterium.

Preferably, Ar₃ and Ara can be each independently any one selected from the group consisting of:

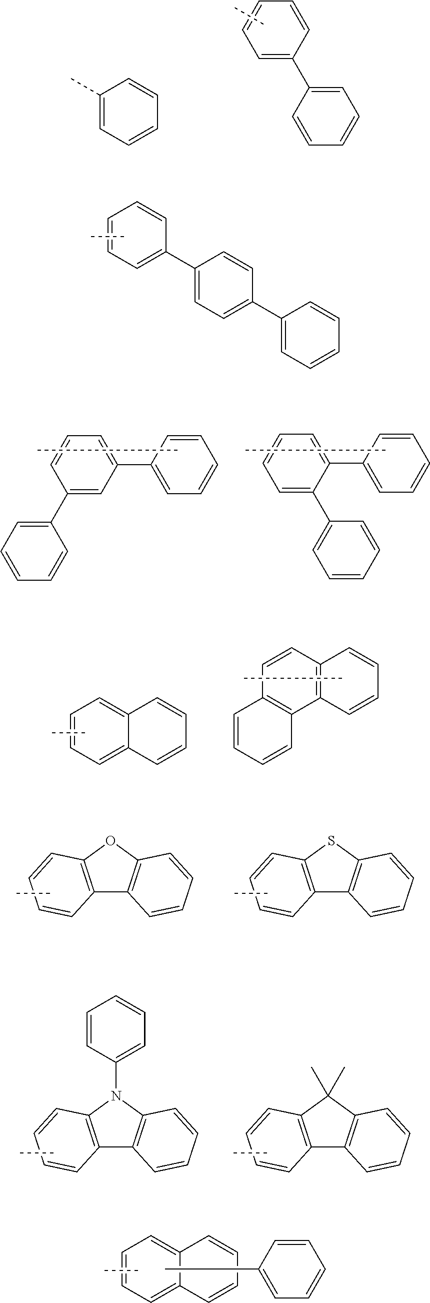

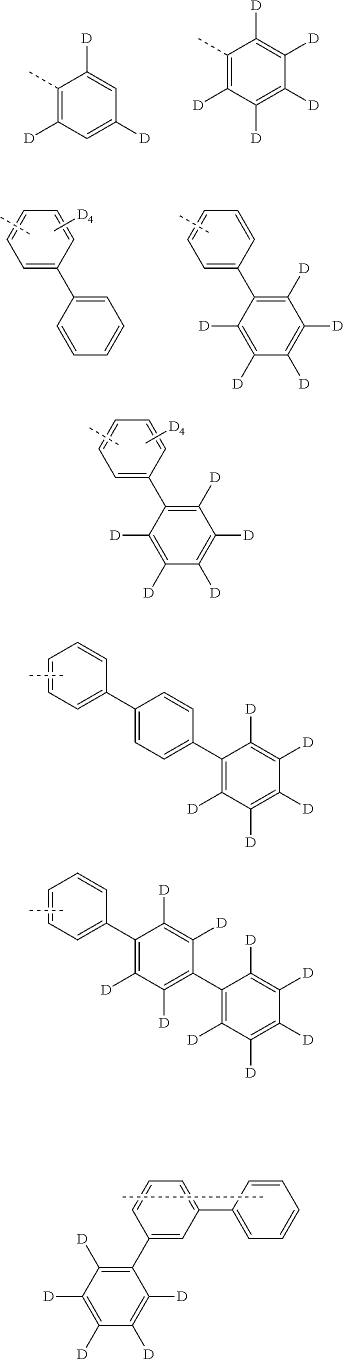

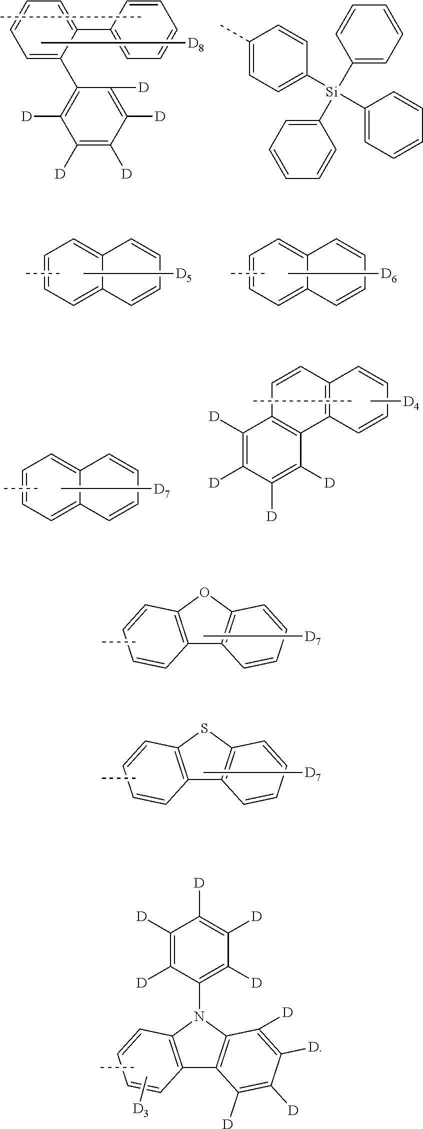

Preferably, L₄ is phenylene, biphenyldiyl, or naphthalenediyl, provided that the phenylene, biphenyldiyl and naphthalenediyl can each be unsubstituted or substituted with deuterium or a C₆₋₆₀ aryl.

More preferably, L₄ can be phenylene, biphenyldiyl, biphenyldiyl substituted with phenyl, or naphthalenediyl; and the hydrogens of L₄ can be each independently unsubstituted or substituted with deuterium,

Preferably, L₄ can be any one selected from the group consisting of:

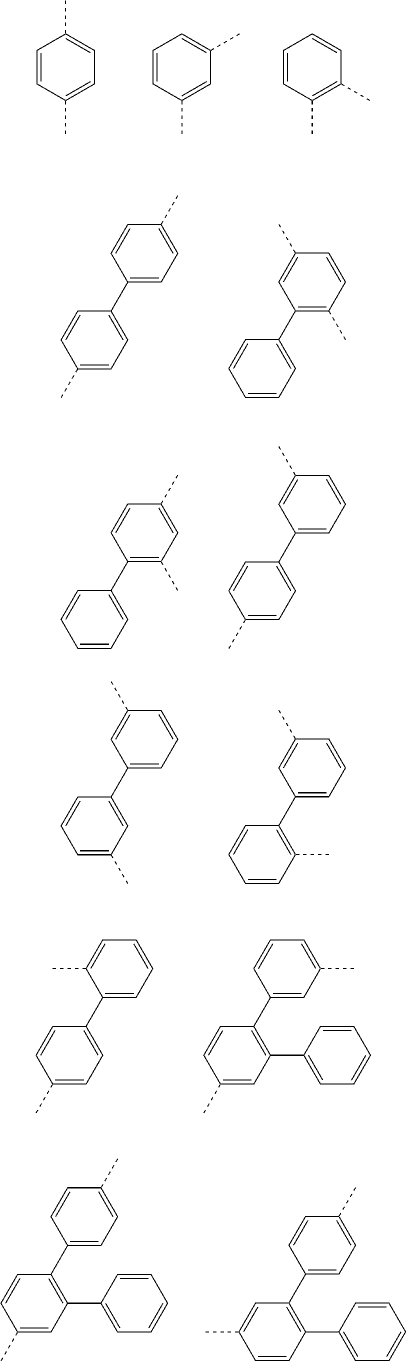

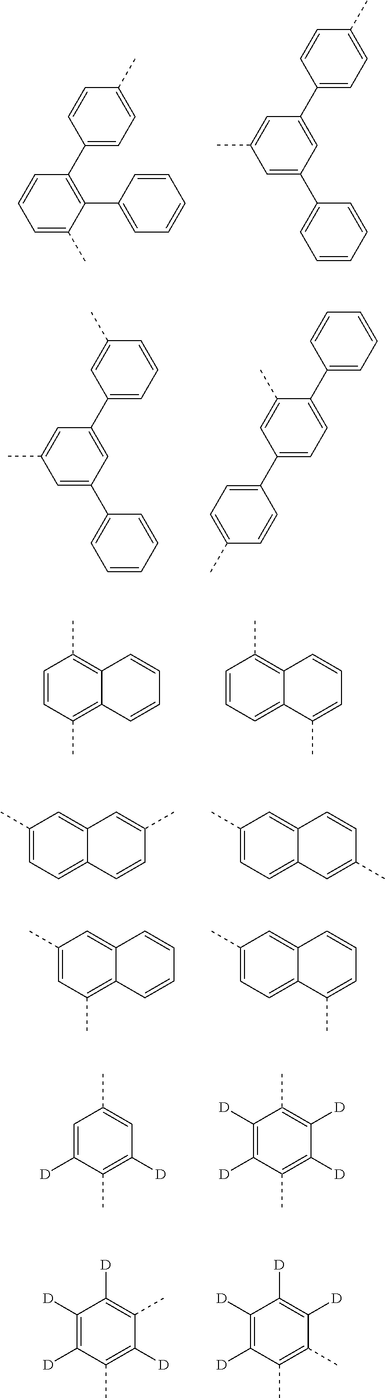

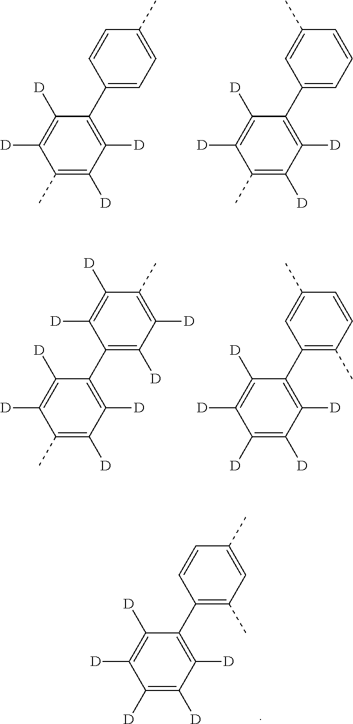

Preferably, L₅ and L₆ are each independently a single bond, a substituted or unsubstituted C₆₋₂₀ arylene, or a substituted or unsubstituted C₂₋₂₀ heteroarylene containing at least one selected from the group consisting of N, O and S.

More preferably, L₅ and L₆ can be each independently a single bond, phenylene, biphenyldiyl, naphthalenediyl, or carbazolediyl, and the hydrogens of L₅ and L₆ can be each independently unsubstituted or substituted with deuterium.

Representative examples of the compound of Chemical Formula 2 are as follows:

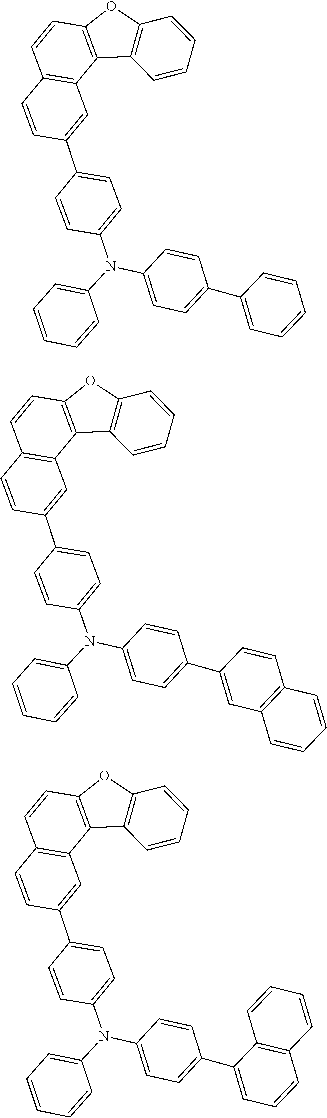

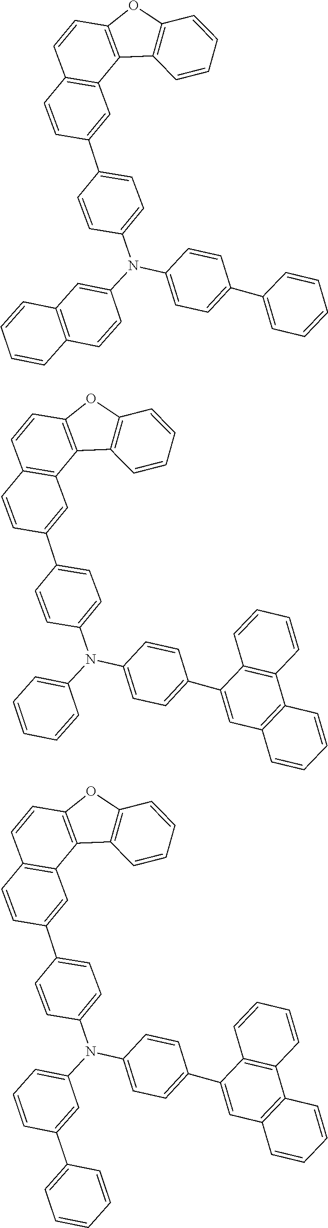 [SMILES: c1ccc(-c2ccc(N(c3ccc(-c4ccc5ccc6oc7ccccc7c6c5c4)cc3)c3ccc4ccccc4c3)cc2)cc1 ; c1ccc(N(c2ccc(-c3ccc4ccc5oc6ccccc6c5c4c3)cc2)c2ccc(-c3cc4ccccc4c4ccccc34)cc2)cc1 ; c1ccc(-c2cccc(N(c3ccc(-c4ccc5ccc6oc7ccccc7c6c5c4)cc3)c3ccc(-c4cc5ccccc5c5ccccc45)cc3)c2)cc1]

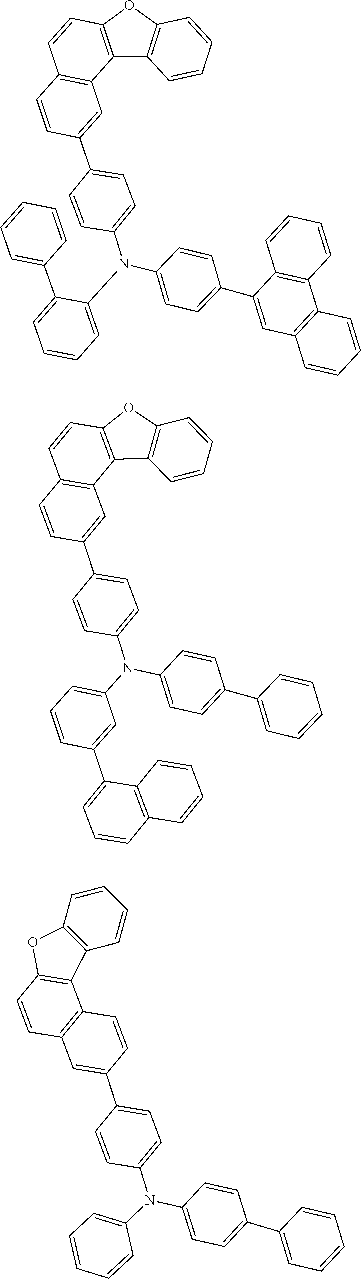 [SMILES: c1ccc(-c2ccccc2N(c2ccc(-c3ccc4ccc5oc6ccccc6c5c4c3)cc2)c2ccc(-c3cc4ccccc4c4ccccc34)cc2)cc1 ; c1ccc(-c2ccc(N(c3ccc(-c4ccc5ccc6oc7ccccc7c6c5c4)cc3)c3cccc(-c4cccc5ccccc45)c3)cc2)cc1 ; c1ccc(-c2ccc(N(c3ccccc3)c3ccc(-c4ccc5c(ccc6oc7ccccc7c65)c4)cc3)cc2)cc1]

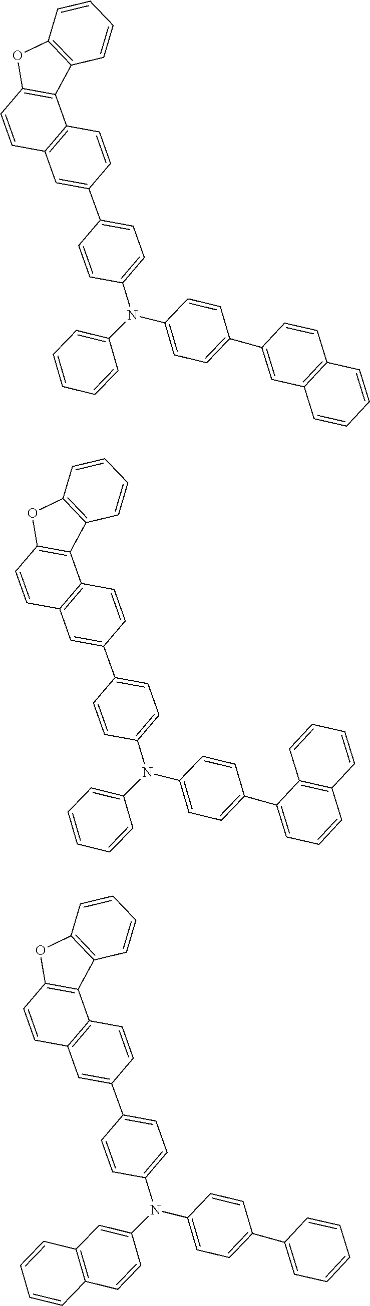

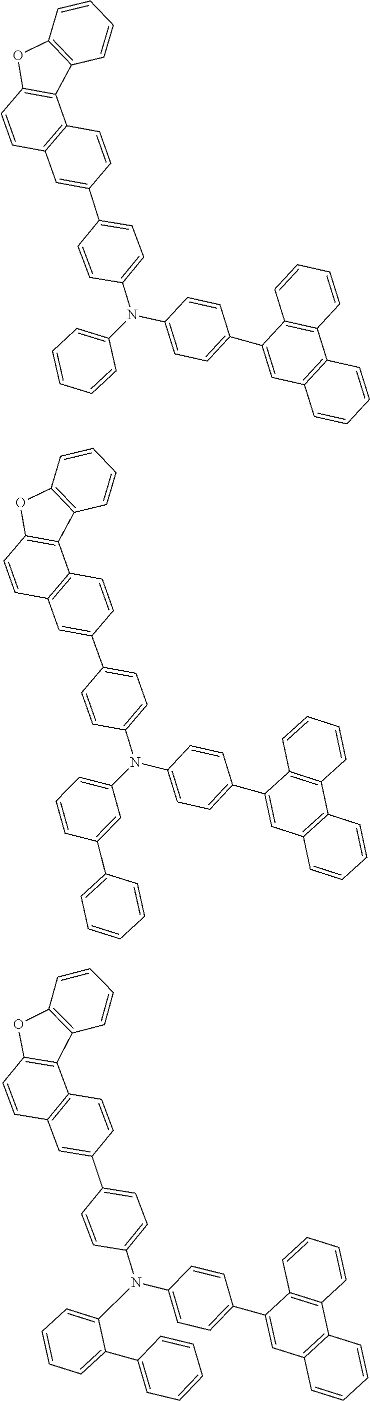

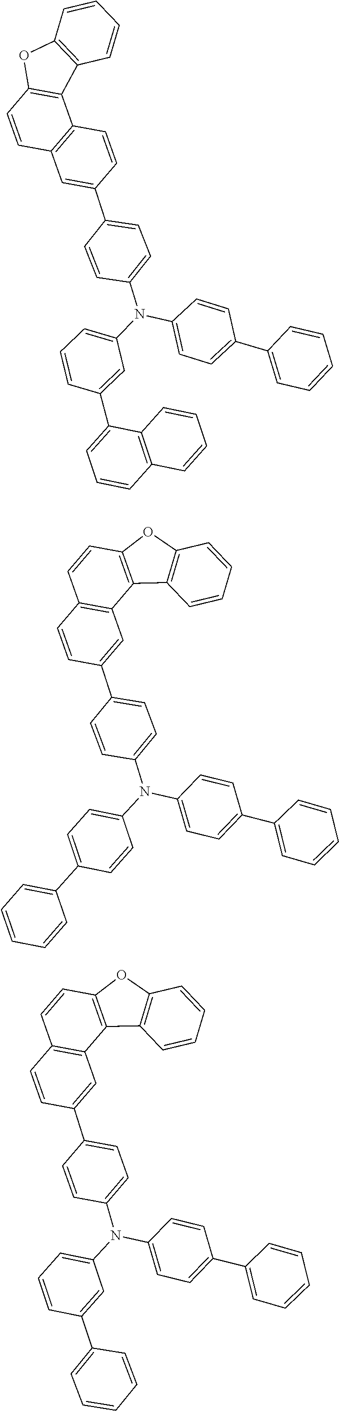

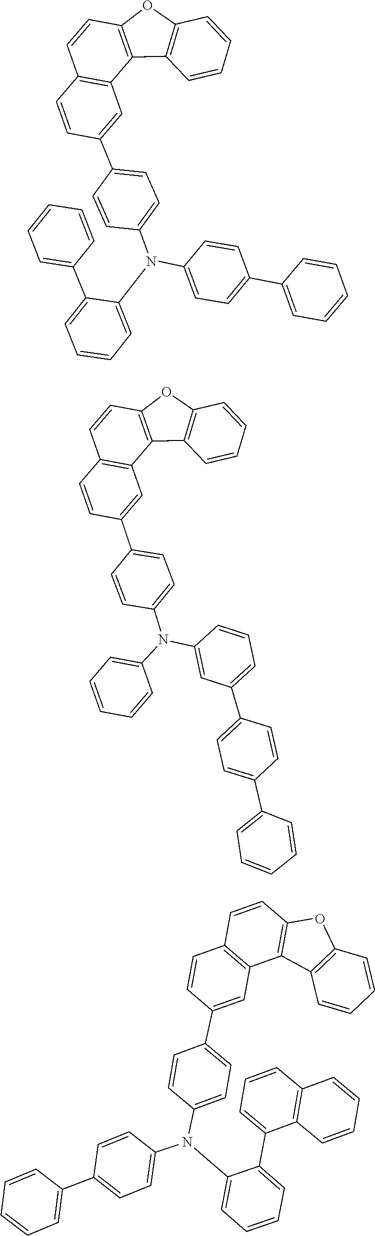

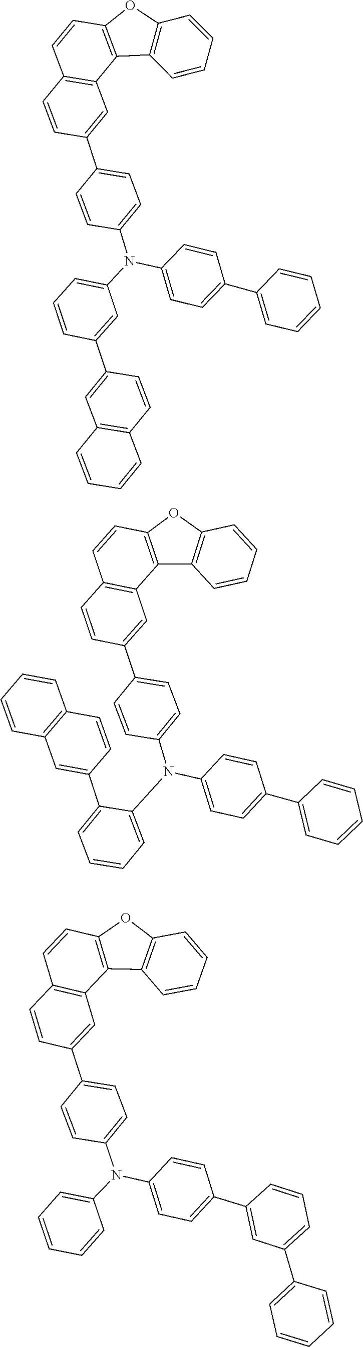

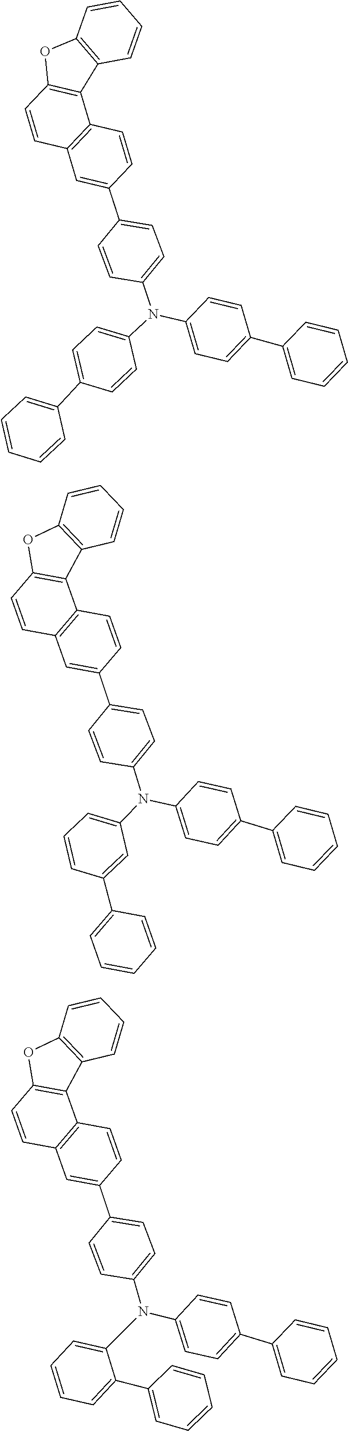

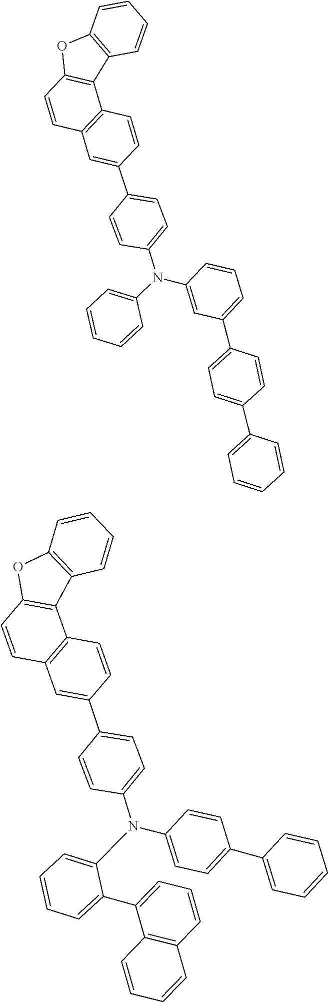 [SMILES: c1ccc(-c2ccc(-c3cccc(N(c4ccccc4)c4ccc(-c5ccc6c(ccc7oc8ccccc8c76)c5)cc4)c3)cc2)cc1 ; c1ccc(-c2ccc(N(c3ccc(-c4ccc5c(ccc6oc7ccccc7c65)c4)cc3)c3ccccc3-c3cccc4ccccc34)cc2)cc1]

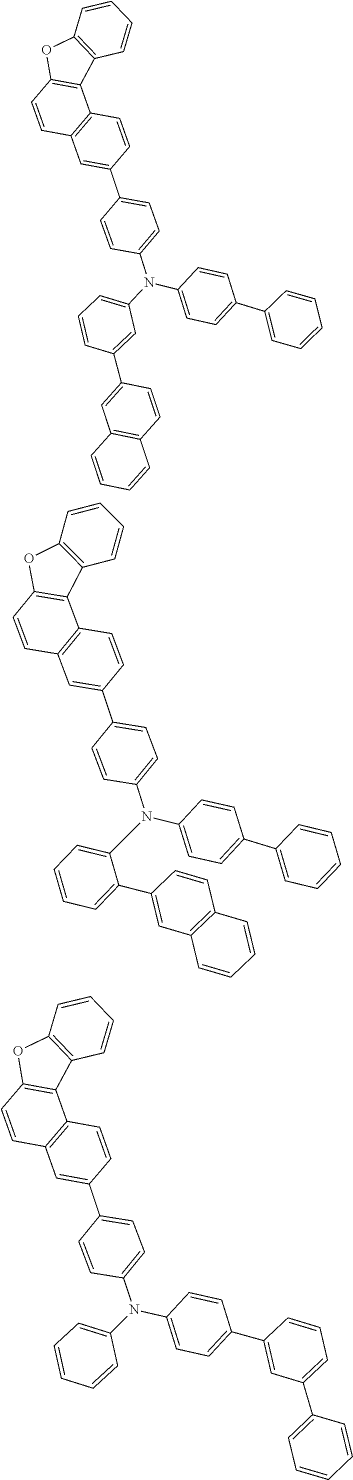

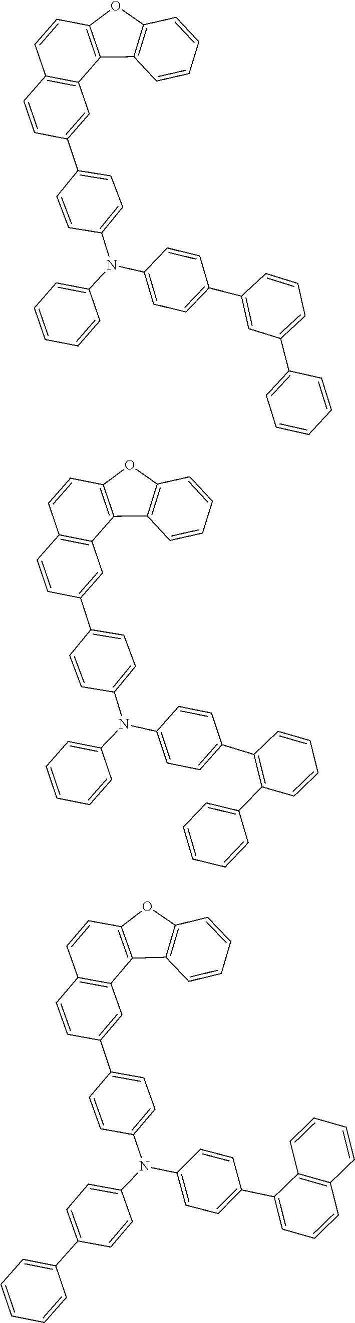 [SMILES: c1ccc(-c2cccc(-c3ccc(N(c4ccccc4)c4ccc(-c5ccc6ccc7oc8ccccc8c7c6c5)cc4)cc3)c2)cc1 ; c1ccc(-c2ccccc2-c2ccc(N(c3ccccc3)c3ccc(-c4ccc5ccc6oc7ccccc7c6c5c4)cc3)cc2)cc1 ; c1ccc(-c2ccc(N(c3ccc(-c4ccc5ccc6oc7ccccc7c6c5c4)cc3)c3ccc(-c4cccc5ccccc45)cc3)cc2)cc1]

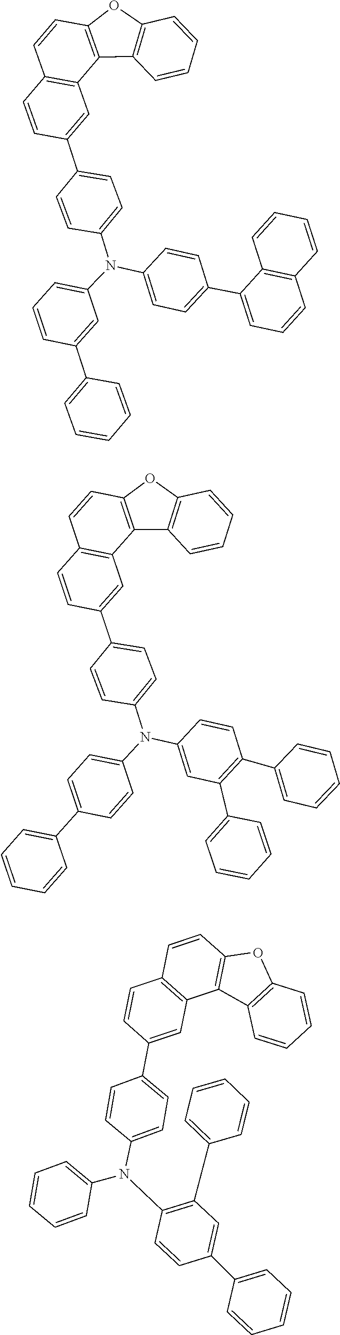 [SMILES: c1ccc(-c2cccc(N(c3ccc(-c4ccc5ccc6oc7ccccc7c6c5c4)cc3)c3ccc(-c4cccc5ccccc45)cc3)c2)cc1 ; c1ccc(-c2ccc(N(c3ccc(-c4ccc5ccc6oc7ccccc7c6c5c4)cc3)c3ccc(-c4ccccc4)c(-c4ccccc4)c3)cc2)cc1 ; c1ccc(-c2ccc(N(c3ccccc3)c3ccc(-c4ccc5ccc6oc7ccccc7c6c5c4)cc3)c(-c3ccccc3)c2)cc1]

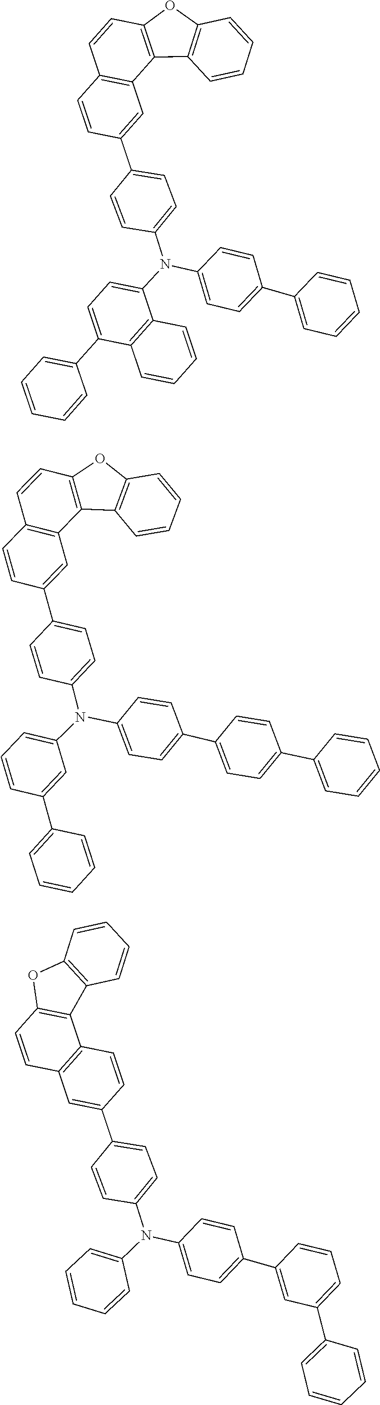

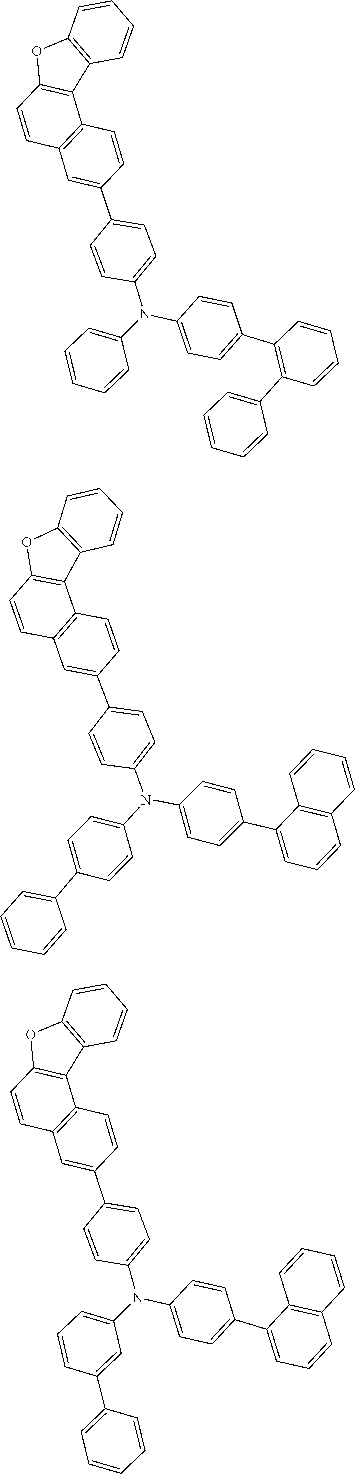

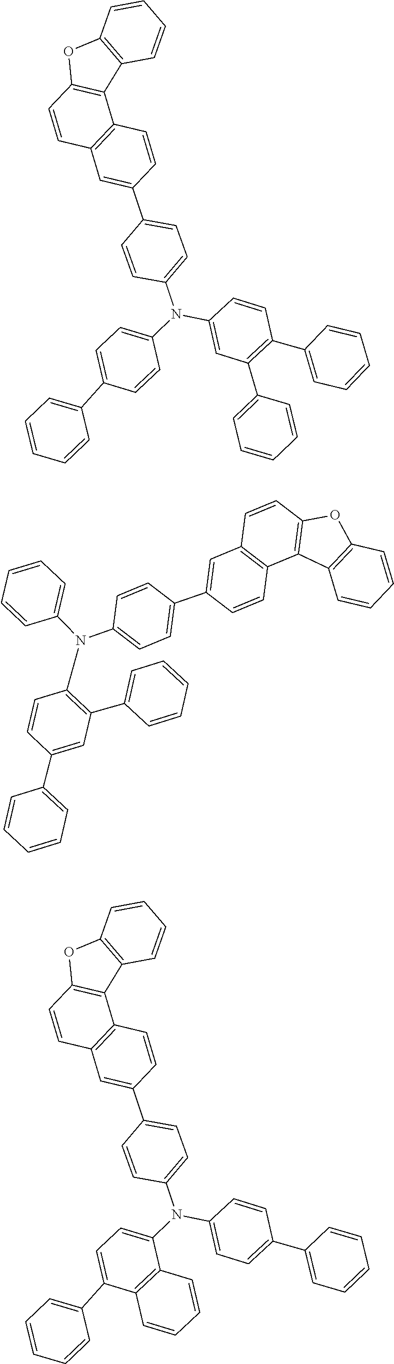

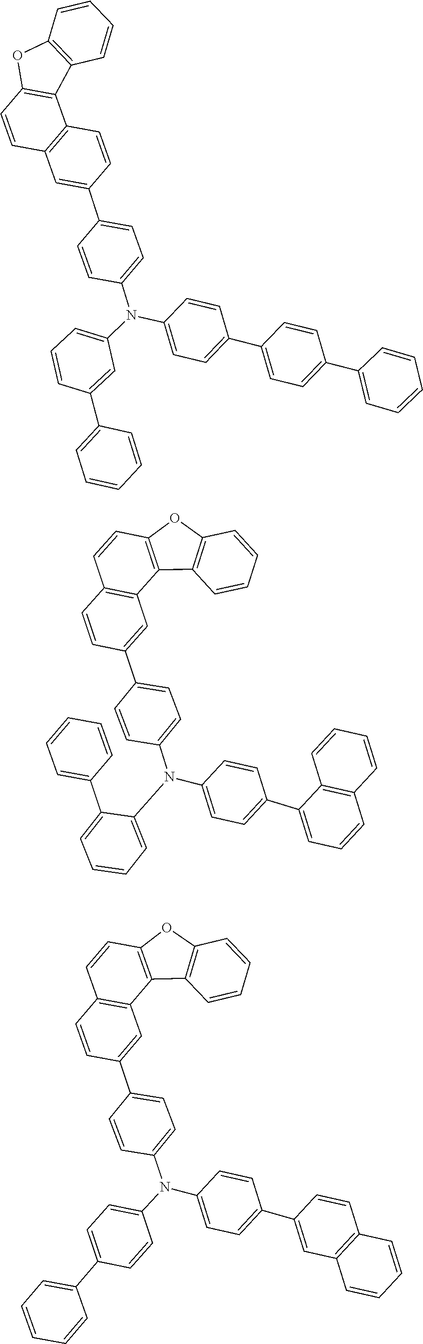

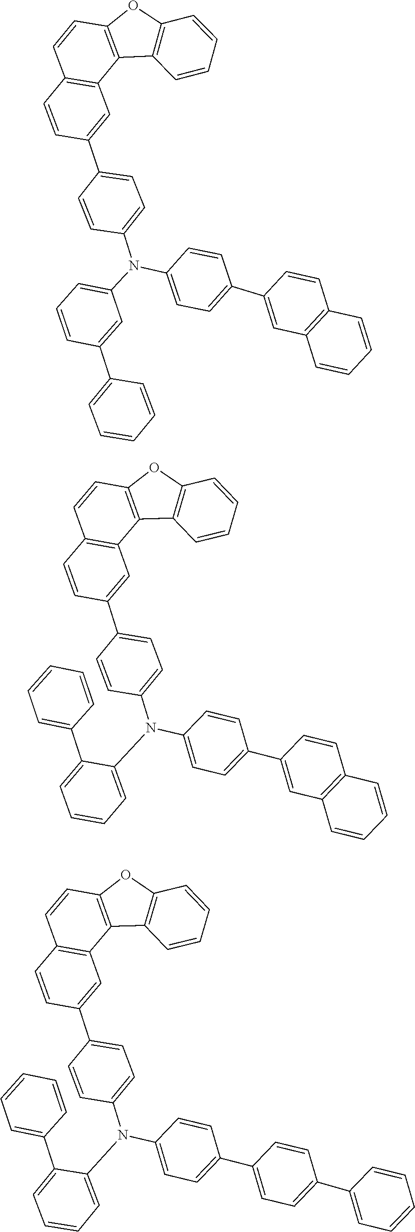

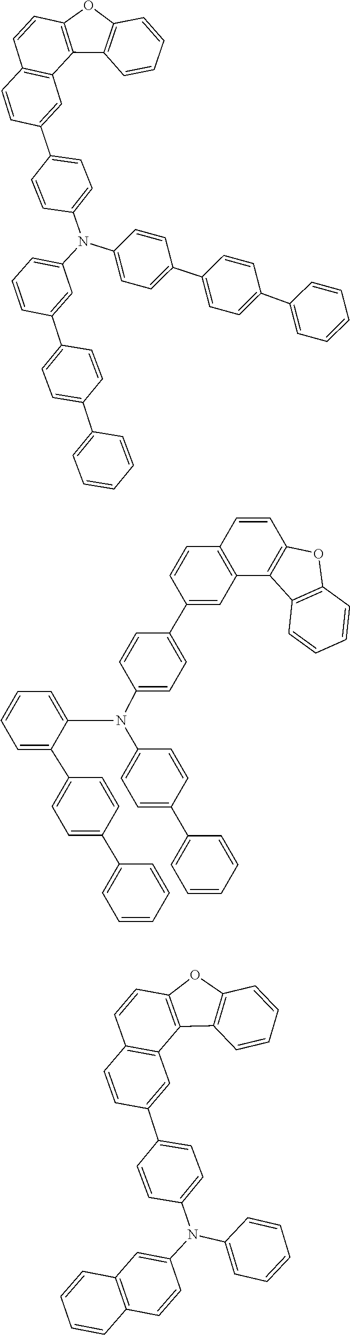 [SMILES: c1ccc(-c2ccc(-c3ccc(N(c4ccc(-c5ccc6ccc7oc8ccccc8c7c6c5)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4ccc5ccc6oc7ccccc7c6c5c4)cc3)cc2)cc1 ; c1ccc(N(c2ccc(-c3ccc4ccc5oc6ccccc6c5c4c3)cc2)c2ccc3ccccc3c2)cc1]

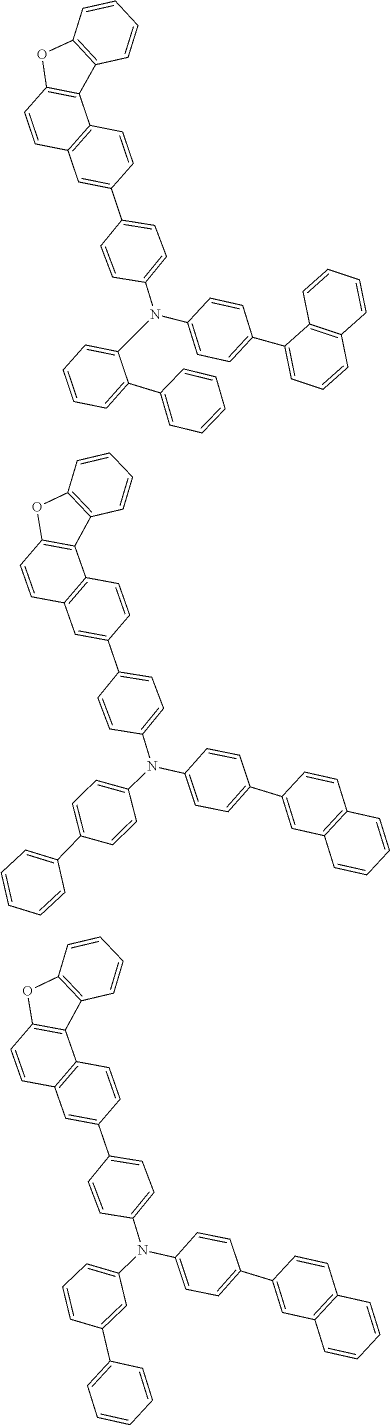

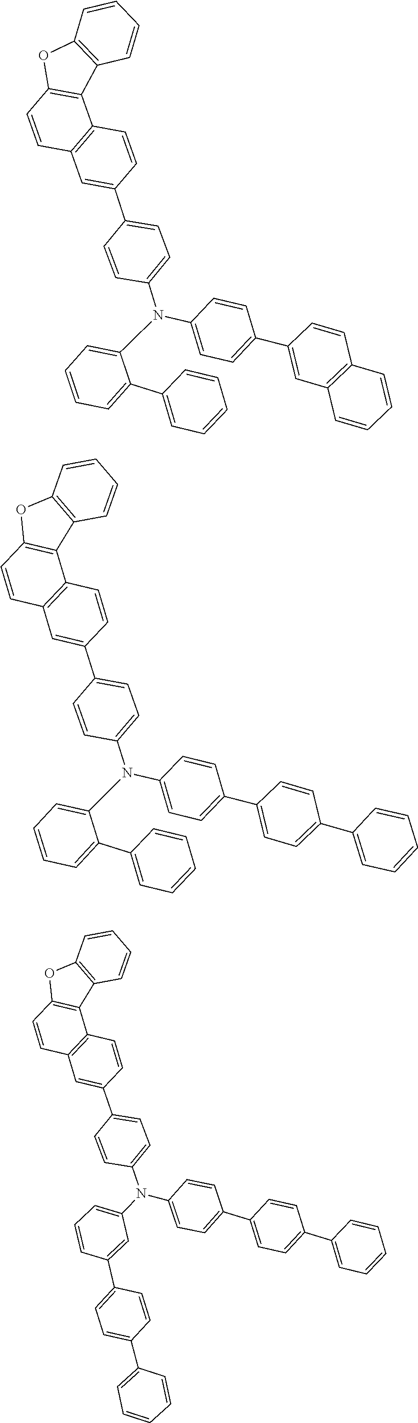 [SMILES: c1ccc(-c2ccccc2N(c2ccc(-c3ccc4ccccc4c3)cc2)c2ccc(-c3ccc4c(ccc5oc6ccccc6c54)c3)cc2)cc1 ; c1ccc(-c2ccc(-c3ccc(N(c4ccc(-c5ccc6c(ccc7oc8ccccc8c76)c5)cc4)c4ccccc4-c4ccccc4)cc3)cc2)cc1 ; c1ccc(-c2ccc(-c3ccc(N(c4ccc(-c5ccc6c(ccc7oc8ccccc8c76)c5)cc4)c4cccc(-c5ccc(-c6ccccc6)cc5)c4)cc3)cc2)cc1]

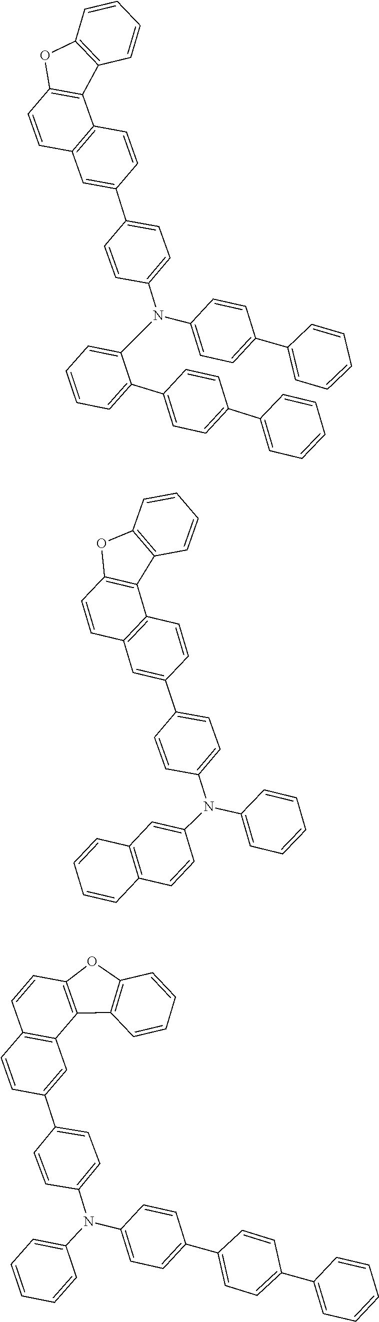 [SMILES: c1ccc(-c2ccc(-c3ccccc3N(c3ccc(-c4ccccc4)cc3)c3ccc(-c4ccc5c(ccc6oc7ccccc7c65)c4)cc3)cc2)cc1 ; c1ccc(N(c2ccc(-c3ccc4c(ccc5oc6ccccc6c54)c3)cc2)c2ccc3ccccc3c2)cc1 ; c1ccc(-c2ccc(-c3ccc(N(c4ccccc4)c4ccc(-c5ccc6ccc7oc8ccccc8c7c6c5)cc4)cc3)cc2)cc1]

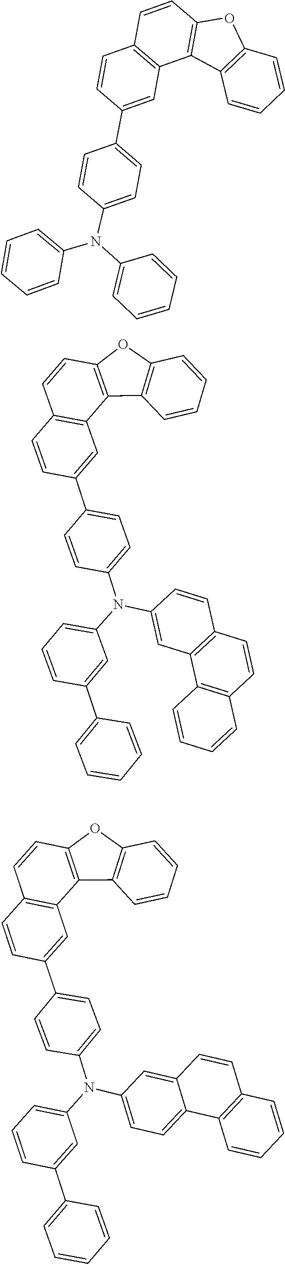

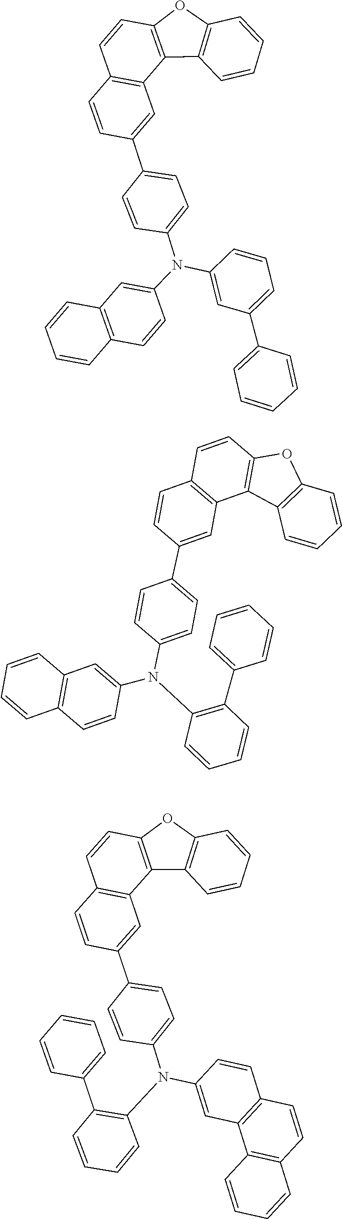

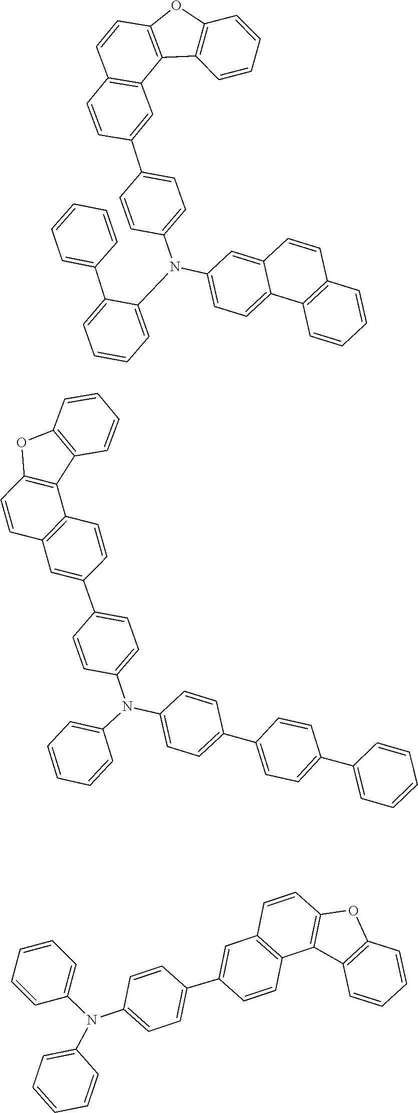

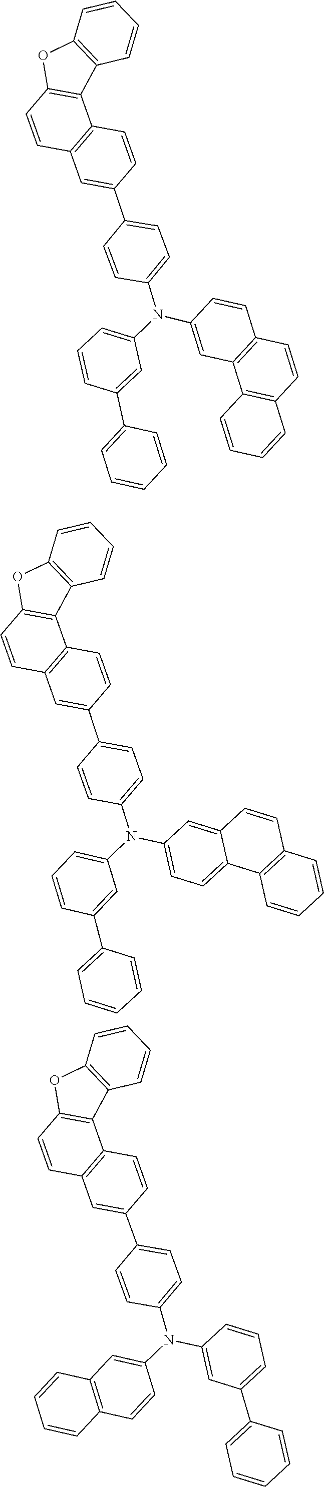

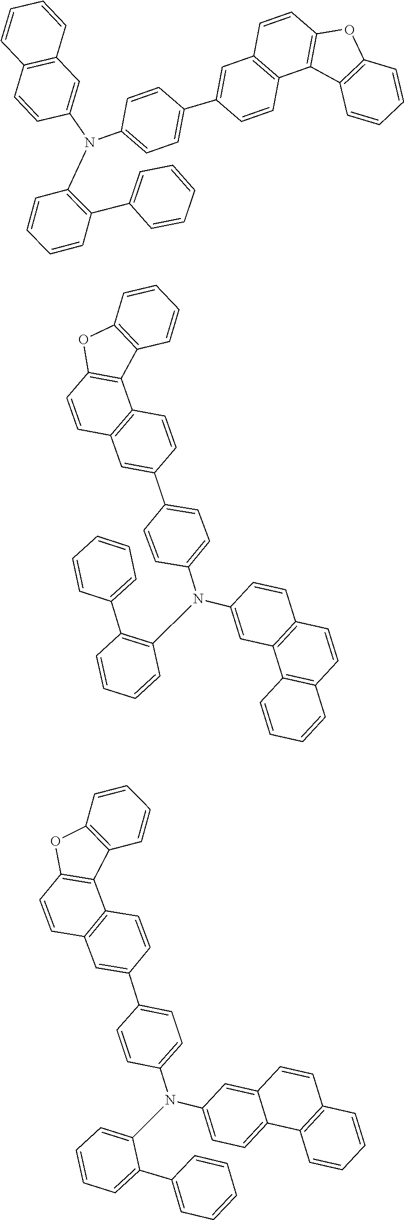 [SMILES: c1ccc(-c2ccccc2N(c2ccc(-c3ccc4c(ccc5oc6ccccc6c54)c3)cc2)c2ccc3ccccc3c2)cc1 ; c1ccc(-c2ccccc2N(c2ccc(-c3ccc4c(ccc5oc6ccccc6c54)c3)cc2)c2ccc3ccc4ccccc4c3c2)cc1 ; c1ccc(-c2ccccc2N(c2ccc(-c3ccc4c(ccc5oc6ccccc6c54)c3)cc2)c2ccc3c(ccc4ccccc43)c2)cc1]

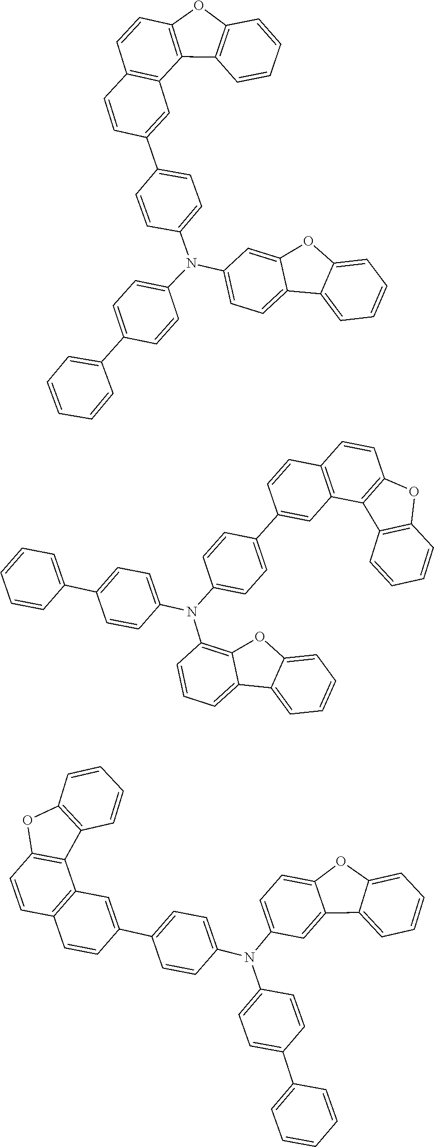 [SMILES: c1ccc(-c2ccc(N(c3ccc(-c4ccc5ccc6oc7ccccc7c6c5c4)cc3)c3ccc4c(c3)oc3ccccc34)cc2)cc1 ; c1ccc(-c2ccc(N(c3ccc(-c4ccc5ccc6oc7ccccc7c6c5c4)cc3)c3cccc4c3oc3ccccc34)cc2)cc1 ; c1ccc(-c2ccc(N(c3ccc(-c4ccc5ccc6oc7ccccc7c6c5c4)cc3)c3ccc4oc5ccccc5c4c3)cc2)cc1]

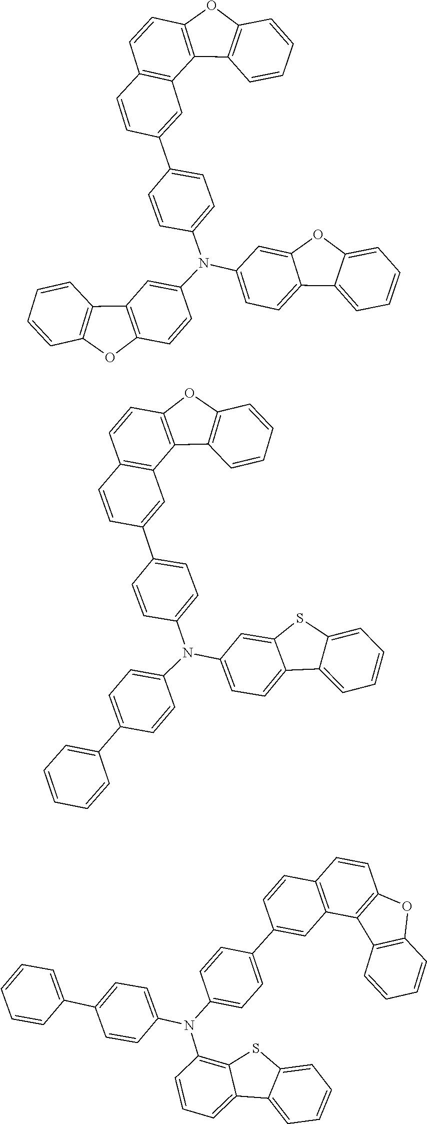 [SMILES: c1ccc2c(c1)oc1cc(N(c3ccc(-c4ccc5ccc6oc7ccccc7c6c5c4)cc3)c3ccc4oc5ccccc5c4c3)ccc12 ; c1ccc(-c2ccc(N(c3ccc(-c4ccc5ccc6oc7ccccc7c6c5c4)cc3)c3ccc4c(c3)sc3ccccc34)cc2)cc1 ; c1ccc(-c2ccc(N(c3ccc(-c4ccc5ccc6oc7ccccc7c6c5c4)cc3)c3cccc4c3sc3ccccc34)cc2)cc1]

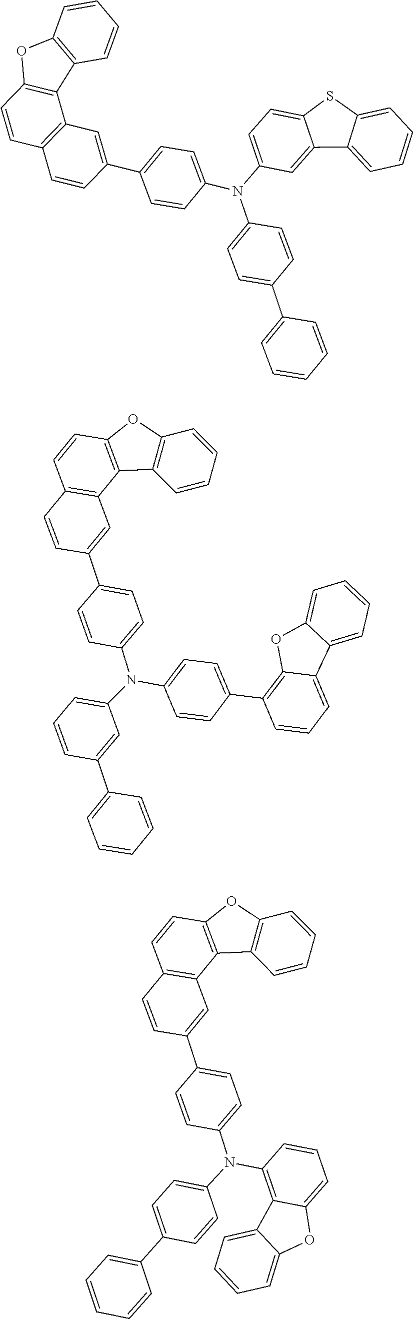 [SMILES: c1ccc(-c2ccc(N(c3ccc(-c4ccc5ccc6oc7ccccc7c6c5c4)cc3)c3ccc4sc5ccccc5c4c3)cc2)cc1 ; c1ccc(-c2cccc(N(c3ccc(-c4ccc5ccc6oc7ccccc7c6c5c4)cc3)c3ccc(-c4cccc5c4oc4ccccc45)cc3)c2)cc1 ; c1ccc(-c2ccc(N(c3ccc(-c4ccc5ccc6oc7ccccc7c6c5c4)cc3)c3cccc4oc5ccccc5c34)cc2)cc1]

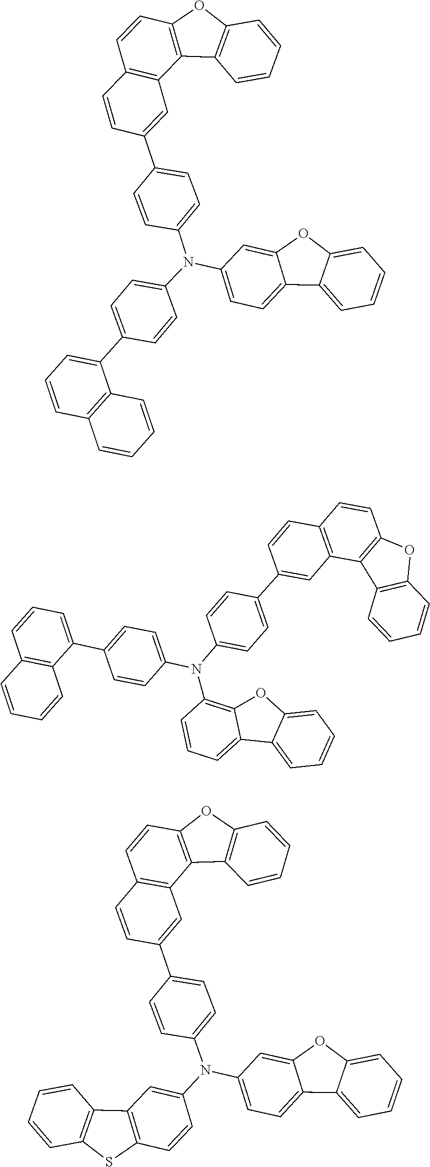 [SMILES: c1ccc2c(-c3ccc(N(c4ccc(-c5ccc6ccc7oc8ccccc8c7c6c5)cc4)c4ccc5c(c4)oc4ccccc45)cc3)cccc2c1 ; c1ccc2c(-c3ccc(N(c4ccc(-c5ccc6ccc7oc8ccccc8c7c6c5)cc4)c4cccc5c4oc4ccccc45)cc3)cccc2c1 ; c1ccc2c(c1)oc1cc(N(c3ccc(-c4ccc5ccc6oc7ccccc7c6c5c4)cc3)c3ccc4sc5ccccc5c4c3)ccc12]

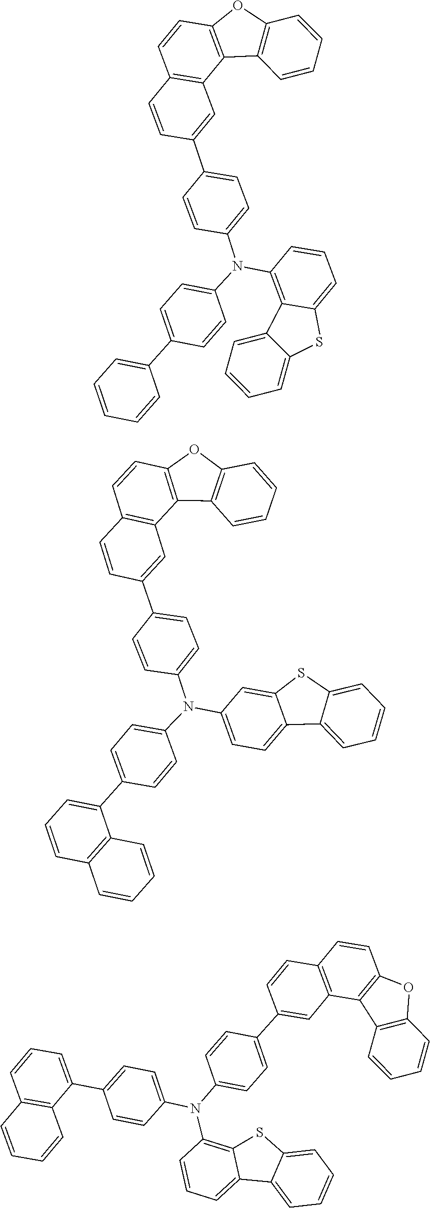

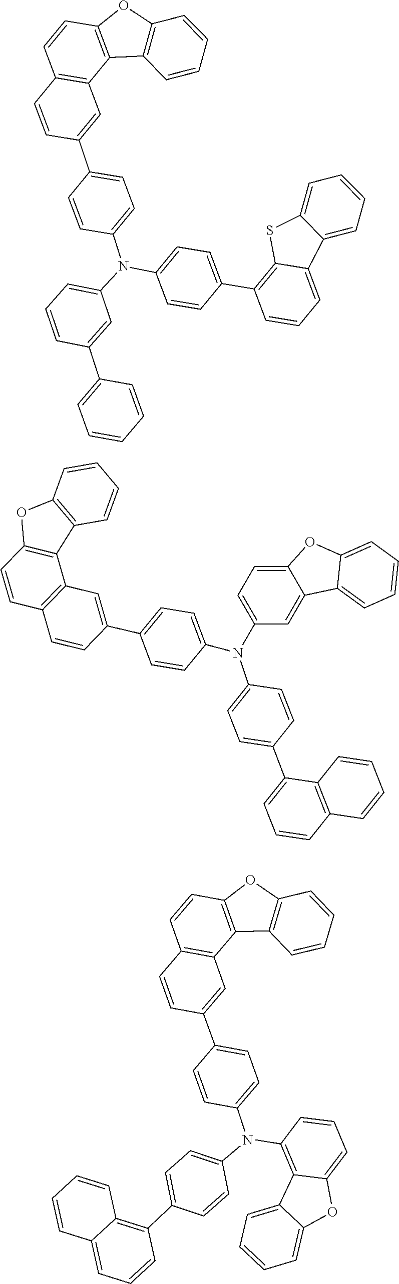

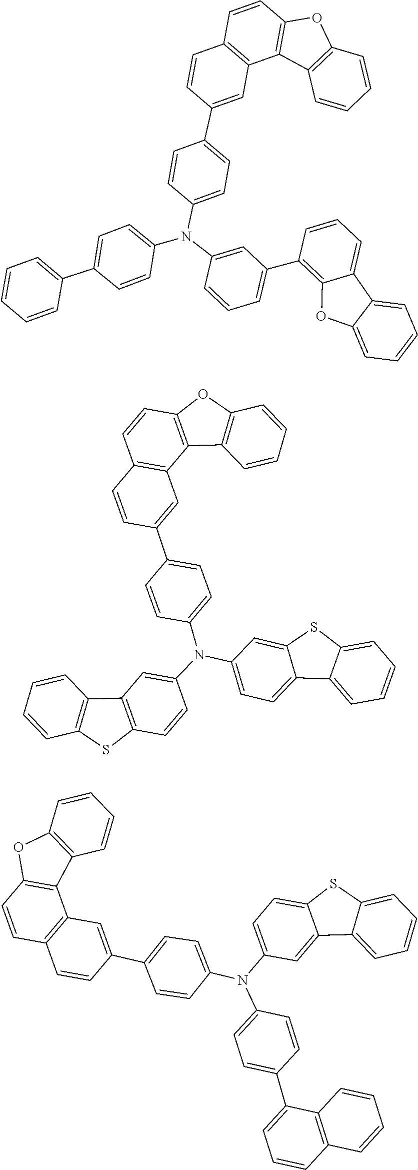

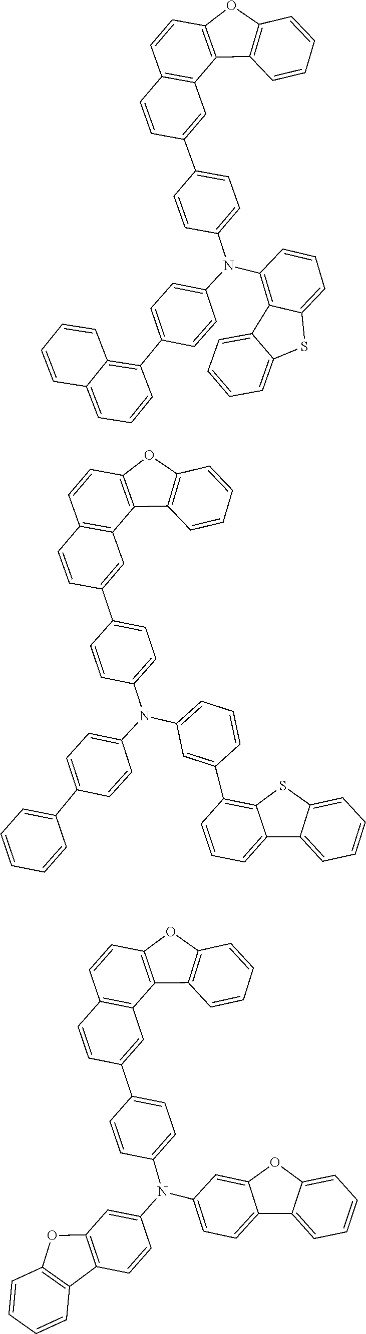

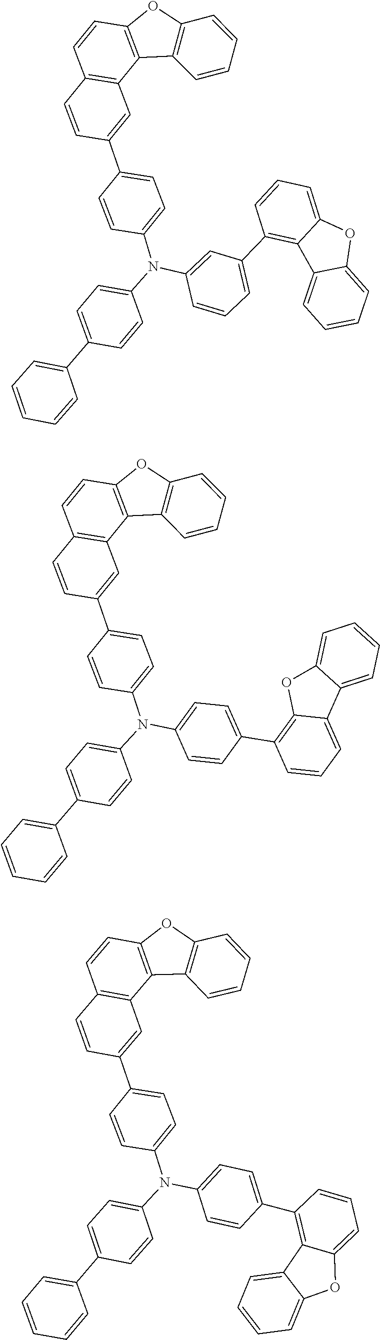 [SMILES: c1ccc(-c2ccc(N(c3ccc(-c4ccc5ccc6oc7ccccc7c6c5c4)cc3)c3cccc(-c4cccc5oc6ccccc6c45)c3)cc2)cc1 ; c1ccc(-c2ccc(N(c3ccc(-c4ccc5ccc6oc7ccccc7c6c5c4)cc3)c3ccc(-c4cccc5c4oc4ccccc45)cc3)cc2)cc1 ; c1ccc(-c2ccc(N(c3ccc(-c4ccc5ccc6oc7ccccc7c6c5c4)cc3)c3ccc(-c4cccc5oc6ccccc6c45)cc3)cc2)cc1]

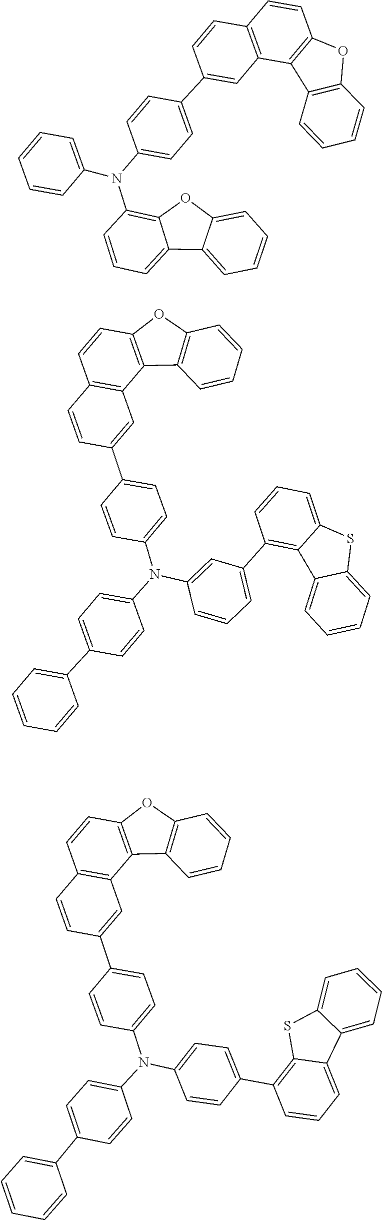

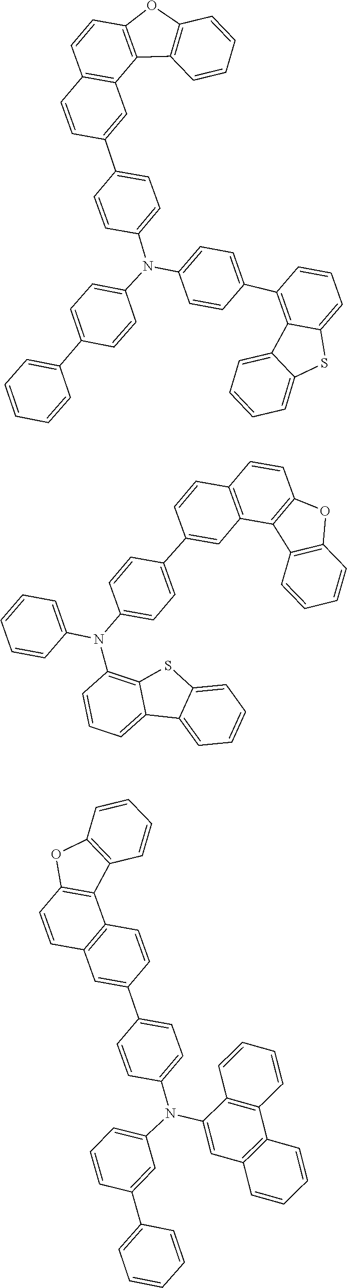

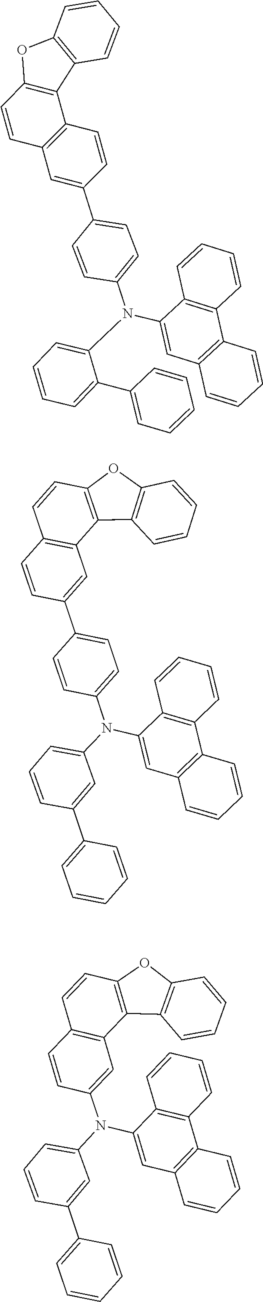 [SMILES: c1ccc(-c2ccccc2N(c2ccc(-c3ccc4c(ccc5oc6ccccc6c54)c3)cc2)c2cc3ccccc3c3ccccc23)cc1 ; c1ccc(-c2cccc(N(c3ccc(-c4ccc5ccc6oc7ccccc7c6c5c4)cc3)c3cc4ccccc4c4ccccc34)c2)cc1 ; c1ccc(-c2cccc(N(c3ccc4ccc5oc6ccccc6c5c4c3)c3cc4ccccc4c4ccccc34)c2)cc1]

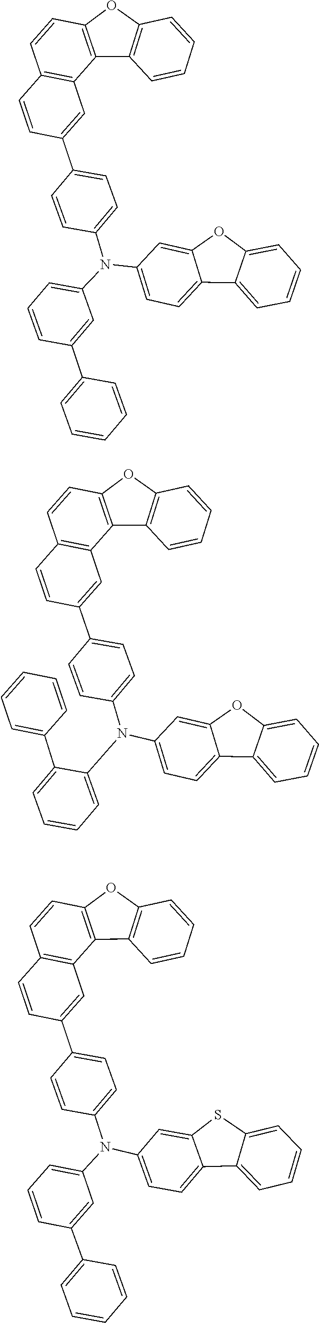

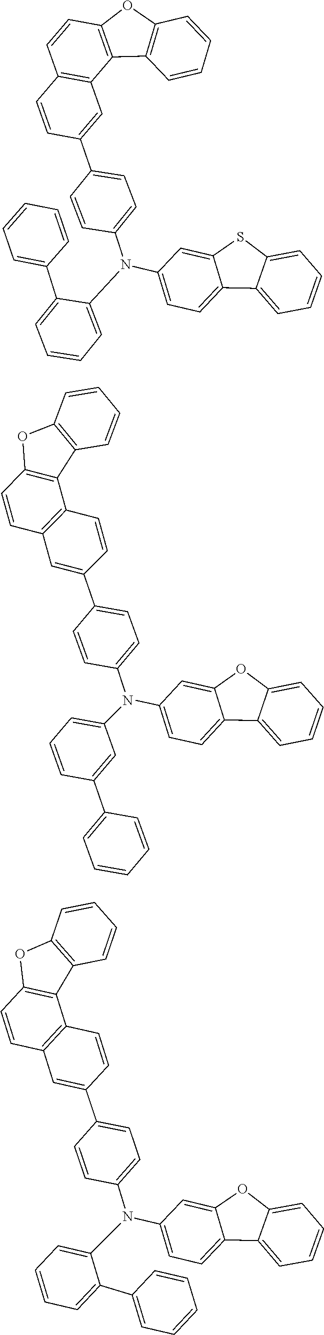

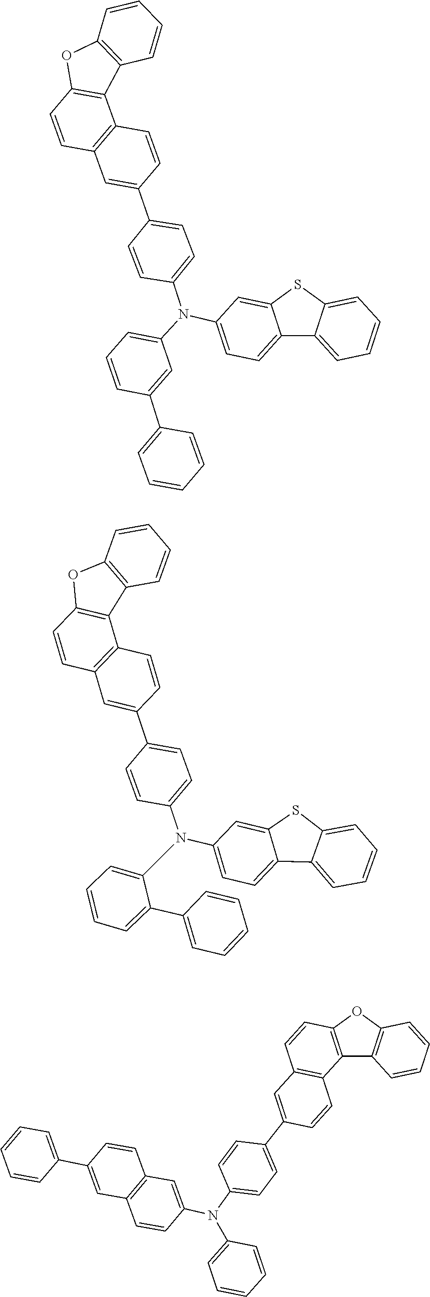

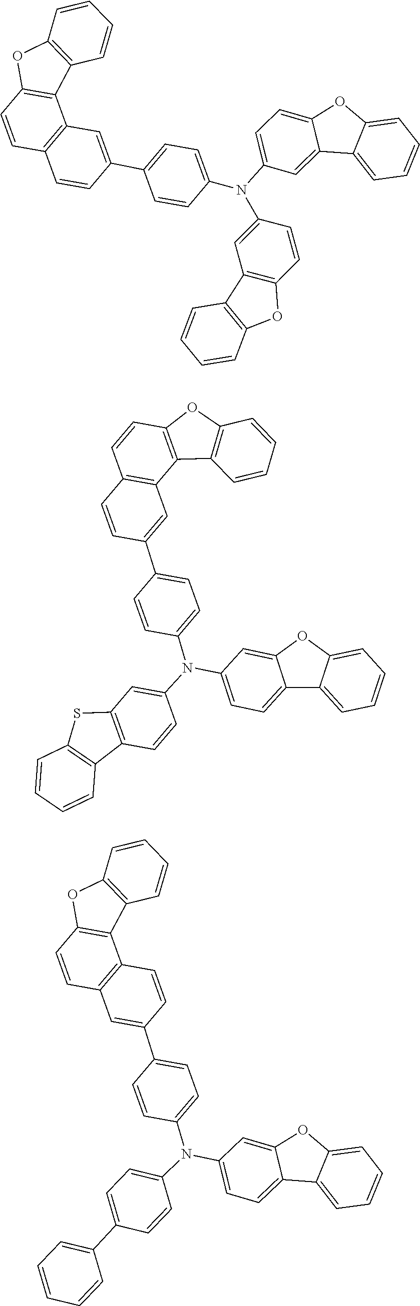

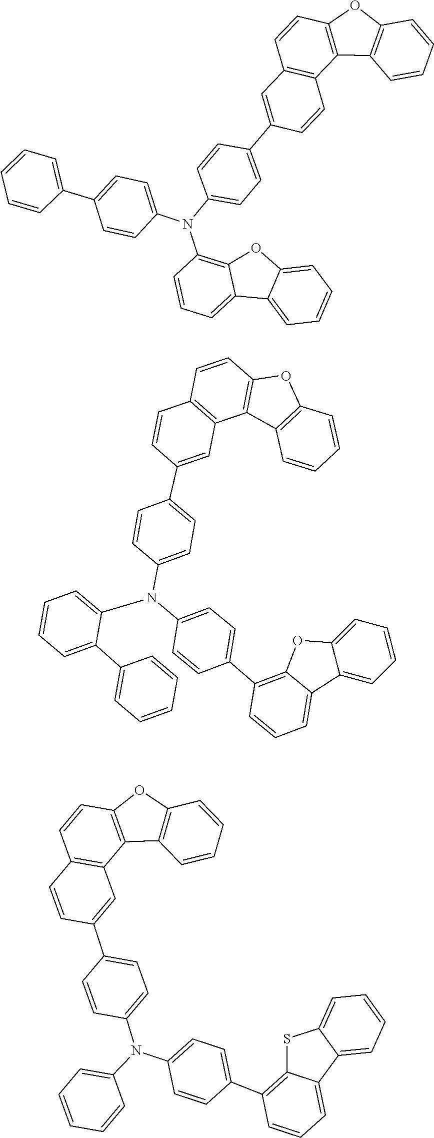

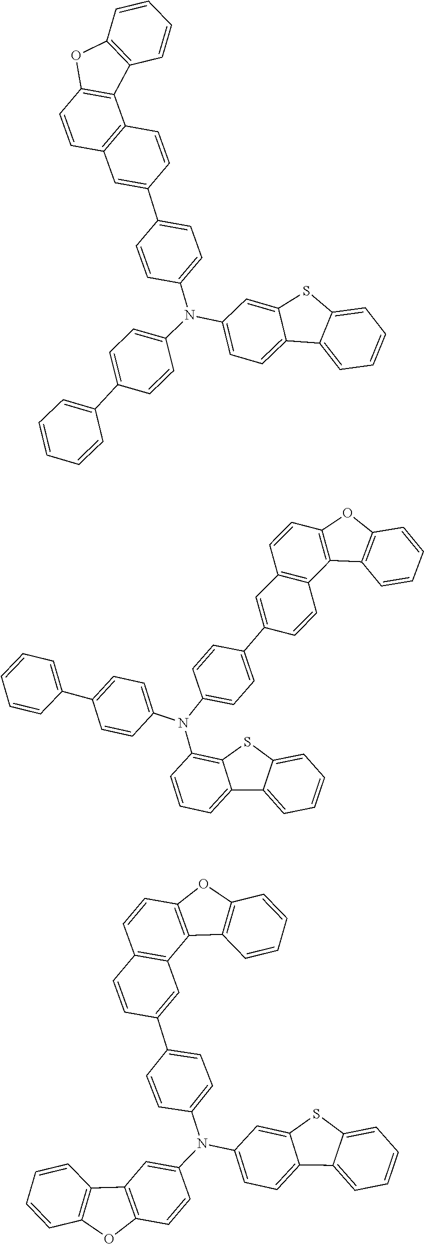 [SMILES: c1ccc(-c2ccc(N(c3ccc(-c4ccc5c(ccc6oc7ccccc7c65)c4)cc3)c3ccc4c(c3)sc3ccccc34)cc2)cc1 ; c1ccc(-c2ccc(N(c3ccc(-c4ccc5c(ccc6oc7ccccc7c65)c4)cc3)c3cccc4c3sc3ccccc34)cc2)cc1 ; c1ccc2c(c1)oc1ccc(N(c3ccc(-c4ccc5ccc6oc7ccccc7c6c5c4)cc3)c3ccc4c(c3)sc3ccccc34)cc12]

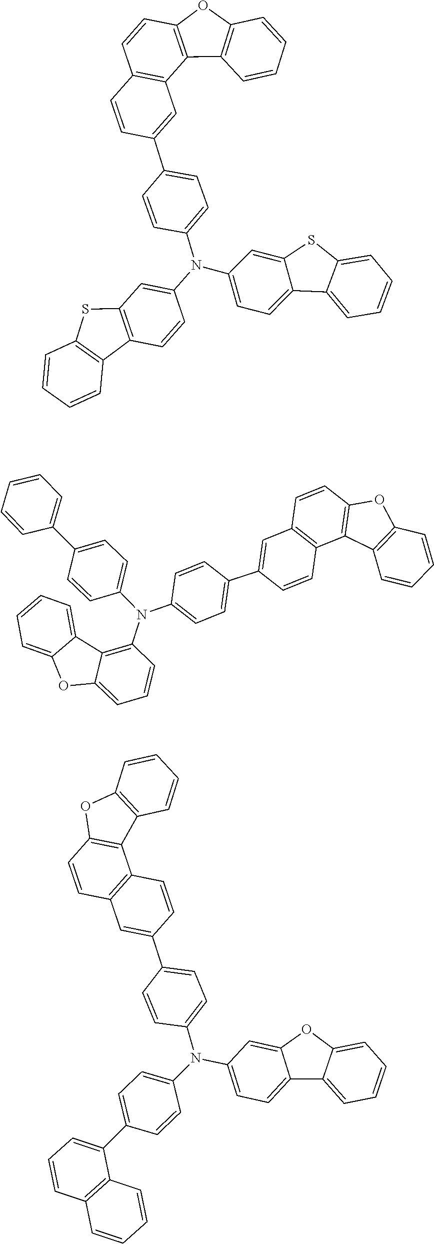 [SMILES: c1ccc2c(c1)oc1ccc3ccc(-c4ccc(N(c5ccc6c(c5)sc5ccccc56)c5ccc6c(c5)sc5ccccc56)cc4)cc3c12 ; c1ccc(-c2ccc(N(c3ccc(-c4ccc5c(ccc6oc7ccccc7c65)c4)cc3)c3cccc4oc5ccccc5c34)cc2)cc1 ; c1ccc2c(-c3ccc(N(c4ccc(-c5ccc6c(ccc7oc8ccccc8c76)c5)cc4)c4ccc5c(c4)oc4ccccc45)cc3)cccc2c1]

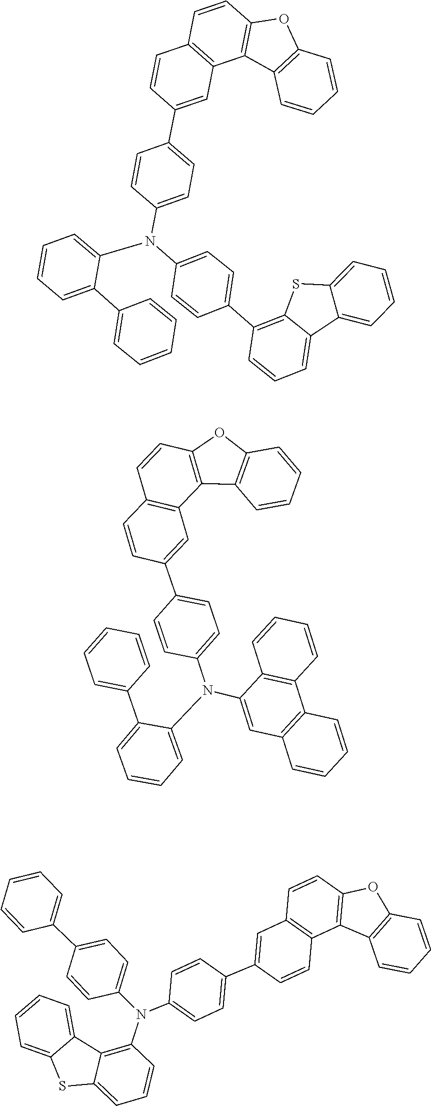 [SMILES: c1ccc(-c2ccccc2N(c2ccc(-c3ccc4ccc5oc6ccccc6c5c4c3)cc2)c2ccc(-c3cccc4c3sc3ccccc34)cc2)cc1 ; c1ccc(-c2ccccc2N(c2ccc(-c3ccc4ccc5oc6ccccc6c5c4c3)cc2)c2cc3ccccc3c3ccccc23)cc1 ; c1ccc(-c2ccc(N(c3ccc(-c4ccc5c(ccc6oc7ccccc7c65)c4)cc3)c3cccc4sc5ccccc5c34)cc2)cc1]

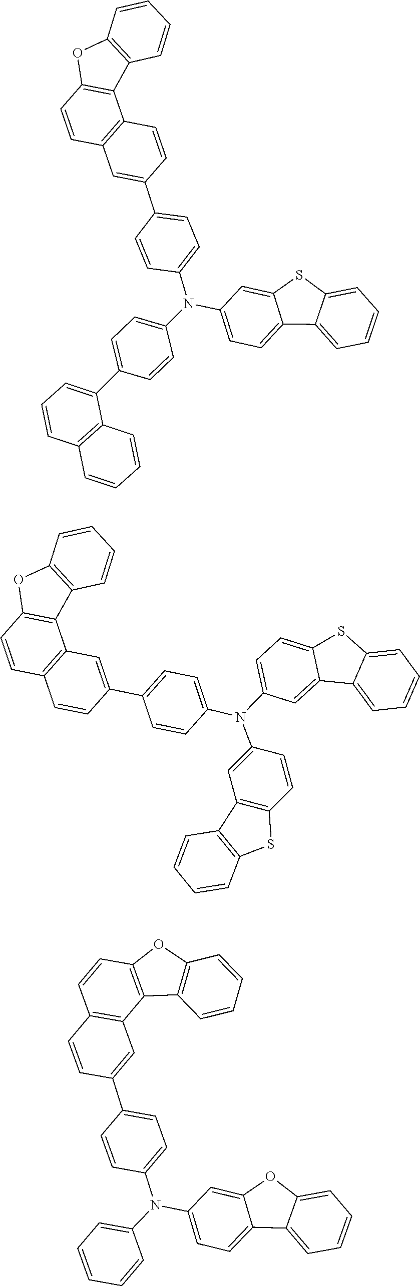

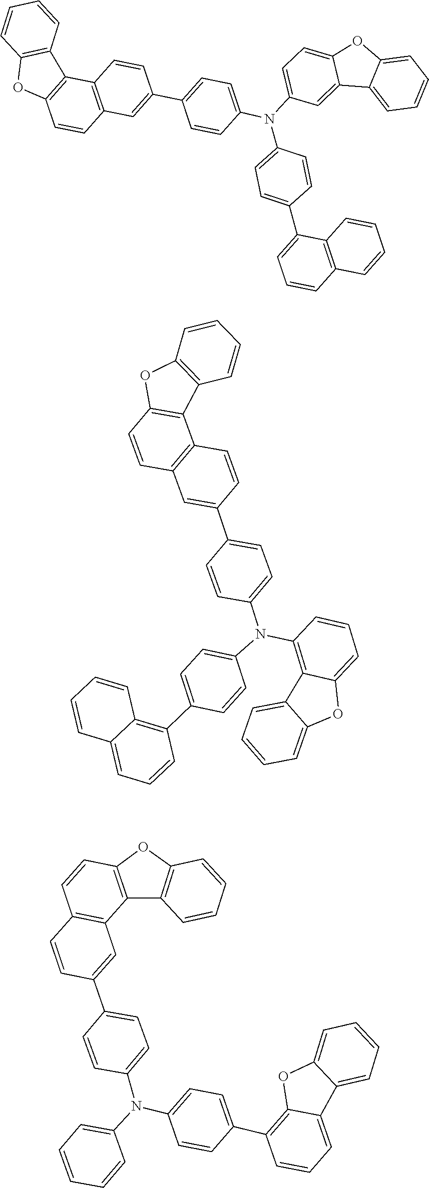 [SMILES: c1ccc2c(-c3ccc(N(c4ccc(-c5ccc6c(ccc7oc8ccccc8c76)c5)cc4)c4ccc5oc6ccccc6c5c4)cc3)cccc2c1 ; c1ccc2c(-c3ccc(N(c4ccc(-c5ccc6c(ccc7oc8ccccc8c76)c5)cc4)c4cccc5oc6ccccc6c45)cc3)cccc2c1 ; c1ccc(N(c2ccc(-c3ccc4ccc5oc6ccccc6c5c4c3)cc2)c2ccc(-c3cccc4c3oc3ccccc34)cc2)cc1]

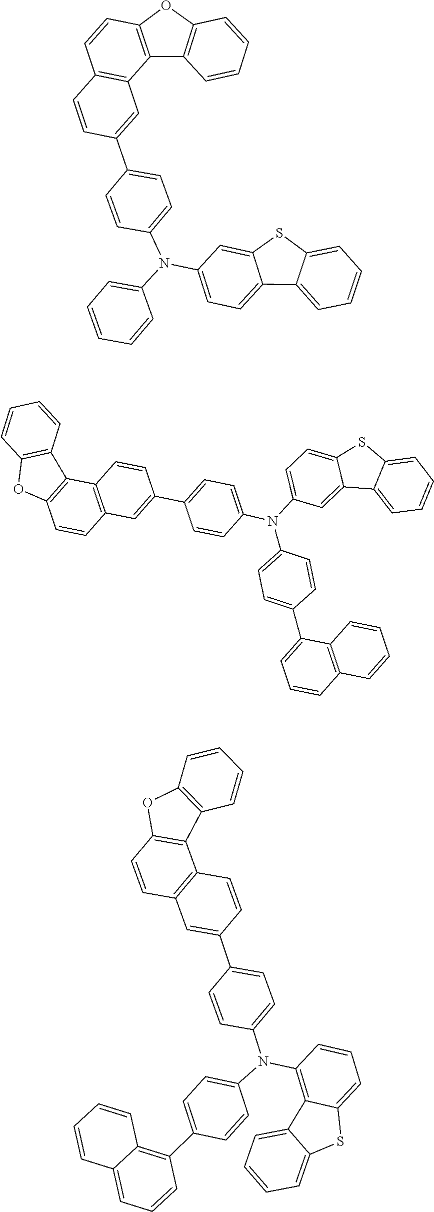 [SMILES: c1ccc(N(c2ccc(-c3ccc4ccc5oc6ccccc6c5c4c3)cc2)c2ccc3c(c2)sc2ccccc23)cc1 ; c1ccc2c(-c3ccc(N(c4ccc(-c5ccc6c(ccc7oc8ccccc8c76)c5)cc4)c4ccc5sc6ccccc6c5c4)cc3)cccc2c1 ; c1ccc2c(-c3ccc(N(c4ccc(-c5ccc6c(ccc7oc8ccccc8c76)c5)cc4)c4cccc5sc6ccccc6c45)cc3)cccc2c1]

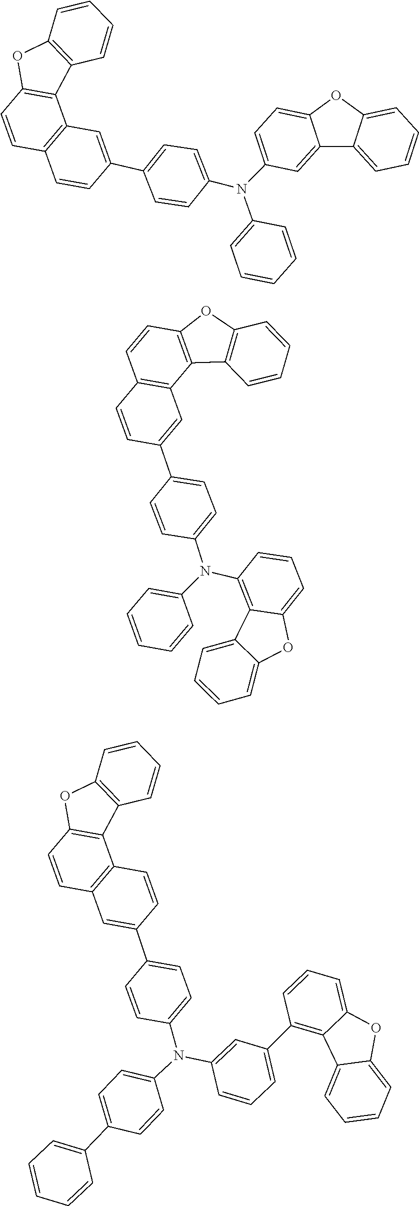

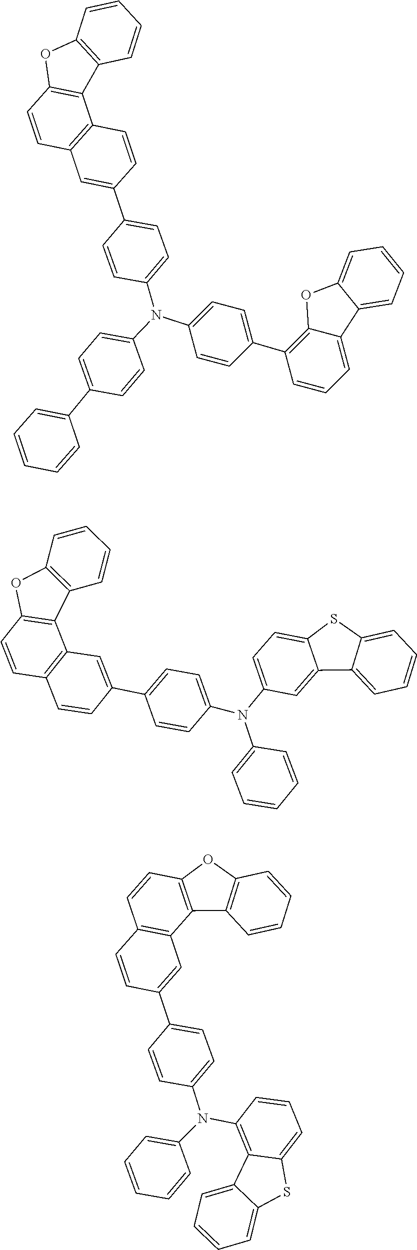 [SMILES: c1ccc(-c2ccc(N(c3ccc(-c4ccc5c(ccc6oc7ccccc7c65)c4)cc3)c3ccc(-c4cccc5c4oc4ccccc45)cc3)cc2)cc1 ; c1ccc(N(c2ccc(-c3ccc4ccc5oc6ccccc6c5c4c3)cc2)c2ccc3sc4ccccc4c3c2)cc1 ; c1ccc(N(c2ccc(-c3ccc4ccc5oc6ccccc6c5c4c3)cc2)c2cccc3sc4ccccc4c23)cc1]

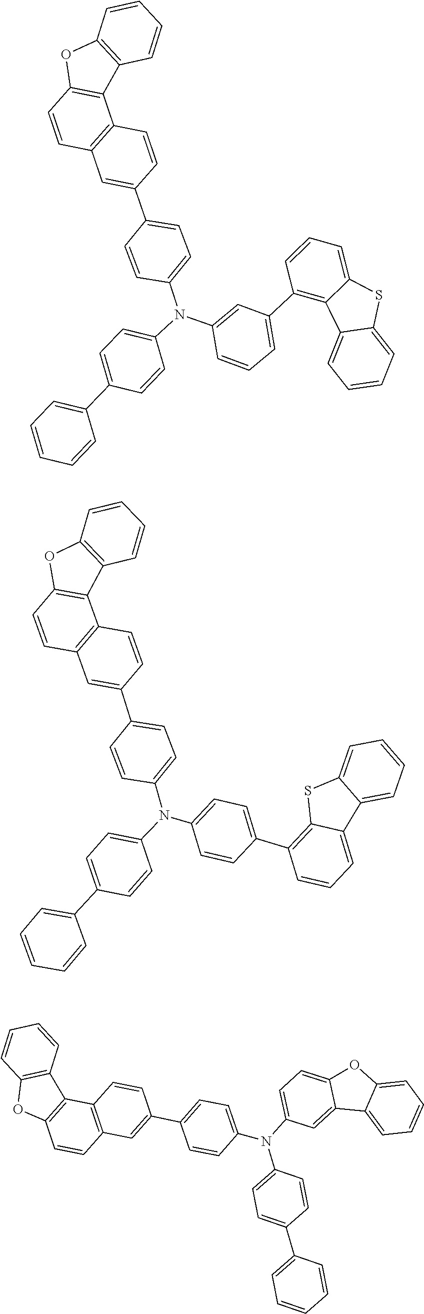 [SMILES: c1ccc(-c2ccc(N(c3ccc(-c4ccc5c(ccc6oc7ccccc7c65)c4)cc3)c3cccc(-c4cccc5sc6ccccc6c45)c3)cc2)cc1 ; c1ccc(-c2ccc(N(c3ccc(-c4ccc5c(ccc6oc7ccccc7c65)c4)cc3)c3ccc(-c4cccc5c4sc4ccccc45)cc3)cc2)cc1 ; c1ccc(-c2ccc(N(c3ccc(-c4ccc5c(ccc6oc7ccccc7c65)c4)cc3)c3ccc4oc5ccccc5c4c3)cc2)cc1]

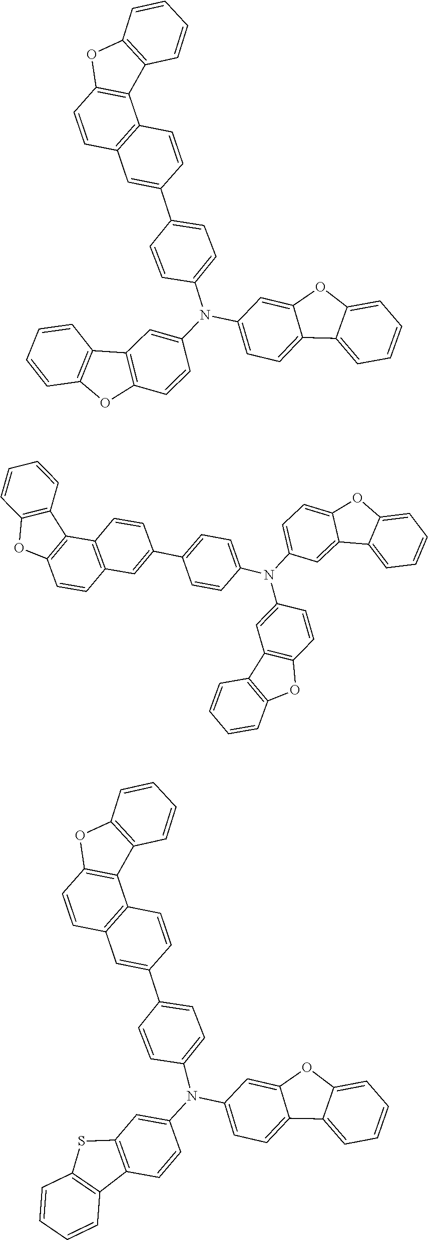 [SMILES: c1ccc2c(c1)oc1cc(N(c3ccc(-c4ccc5c(ccc6oc7ccccc7c65)c4)cc3)c3ccc4oc5ccccc5c4c3)ccc12 ; c1ccc2c(c1)oc1ccc(N(c3ccc(-c4ccc5c(ccc6oc7ccccc7c65)c4)cc3)c3ccc4oc5ccccc5c4c3)cc12 ; c1ccc2c(c1)oc1cc(N(c3ccc(-c4ccc5c(ccc6oc7ccccc7c65)c4)cc3)c3ccc4c(c3)sc3ccccc34)ccc12]

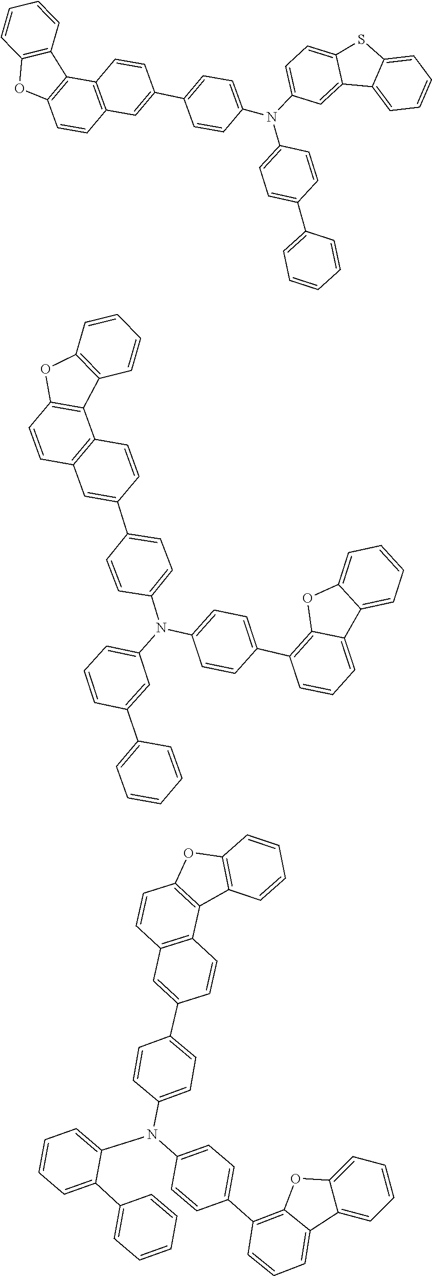 [SMILES: c1ccc(-c2ccc(N(c3ccc(-c4ccc5c(ccc6oc7ccccc7c65)c4)cc3)c3ccc4sc5ccccc5c4c3)cc2)cc1 ; c1ccc(-c2cccc(N(c3ccc(-c4ccc5c(ccc6oc7ccccc7c65)c4)cc3)c3ccc(-c4cccc5c4oc4ccccc45)cc3)c2)cc1 ; c1ccc(-c2ccccc2N(c2ccc(-c3ccc4c(ccc5oc6ccccc6c54)c3)cc2)c2ccc(-c3cccc4c3oc3ccccc34)cc2)cc1]

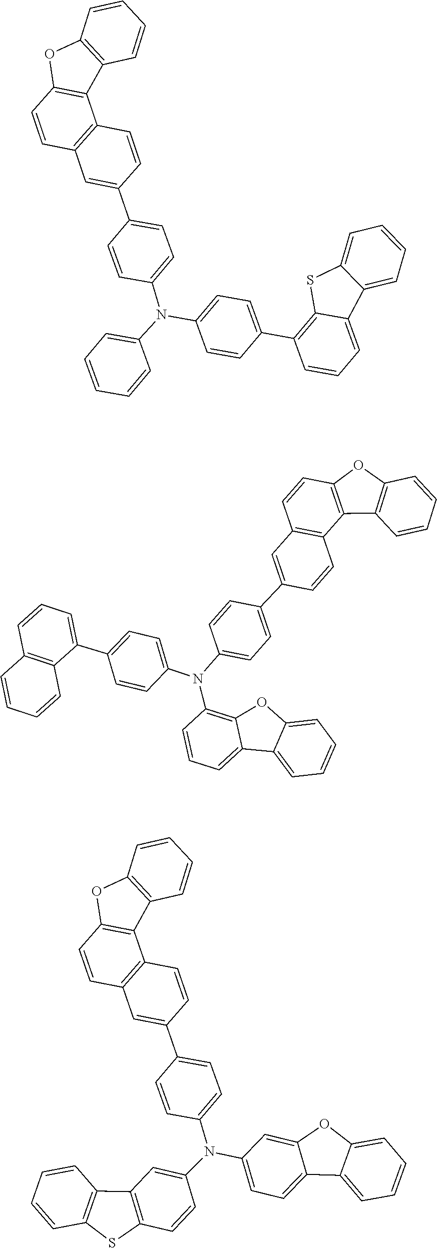 [SMILES: c1ccc(N(c2ccc(-c3ccc4c(ccc5oc6ccccc6c54)c3)cc2)c2ccc(-c3cccc4c3sc3ccccc34)cc2)cc1 ; c1ccc2c(-c3ccc(N(c4ccc(-c5ccc6c(ccc7oc8ccccc8c76)c5)cc4)c4cccc5c4oc4ccccc45)cc3)cccc2c1 ; c1ccc2c(c1)oc1cc(N(c3ccc(-c4ccc5c(ccc6oc7ccccc7c65)c4)cc3)c3ccc4sc5ccccc5c4c3)ccc12]

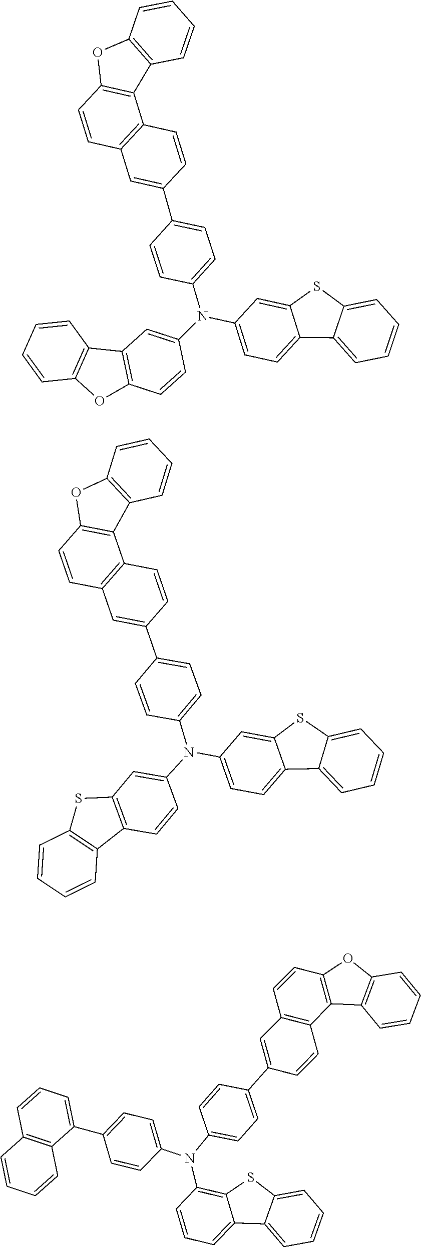

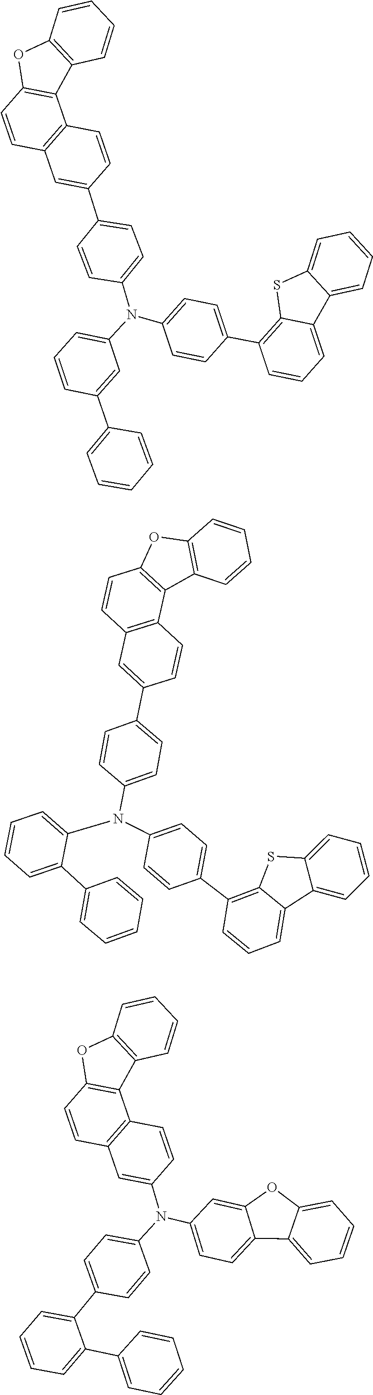 [SMILES: c1ccc(-c2cccc(N(c3ccc(-c4ccc5c(ccc6oc7ccccc7c65)c4)cc3)c3ccc(-c4cccc5c4sc4ccccc45)cc3)c2)cc1 ; c1ccc(-c2ccccc2N(c2ccc(-c3ccc4c(ccc5oc6ccccc6c54)c3)cc2)c2ccc(-c3cccc4c3sc3ccccc34)cc2)cc1 ; c1ccc(-c2ccccc2-c2ccc(N(c3ccc4c(ccc5oc6ccccc6c54)c3)c3ccc4c(c3)oc3ccccc34)cc2)cc1]

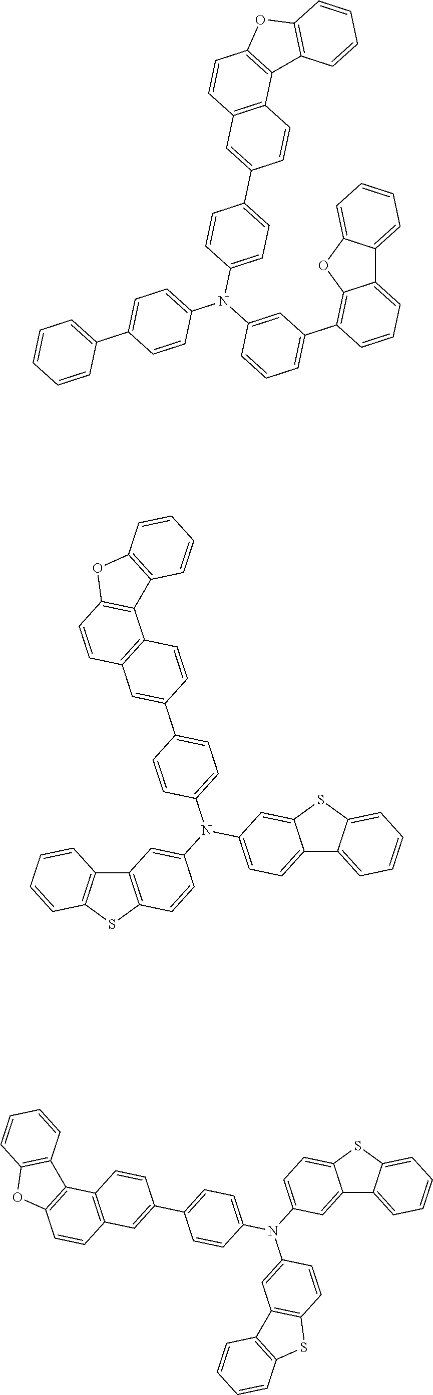 [SMILES: c1ccc(-c2ccc(N(c3ccc(-c4ccc5c(ccc6oc7ccccc7c65)c4)cc3)c3cccc(-c4cccc5c4oc4ccccc45)c3)cc2)cc1 ; c1ccc2c(c1)oc1ccc3cc(-c4ccc(N(c5ccc6c(c5)sc5ccccc56)c5ccc6sc7ccccc7c6c5)cc4)ccc3c12 ; c1ccc2c(c1)oc1ccc3cc(-c4ccc(N(c5ccc6sc7ccccc7c6c5)c5ccc6sc7ccccc7c6c5)cc4)ccc3c12]

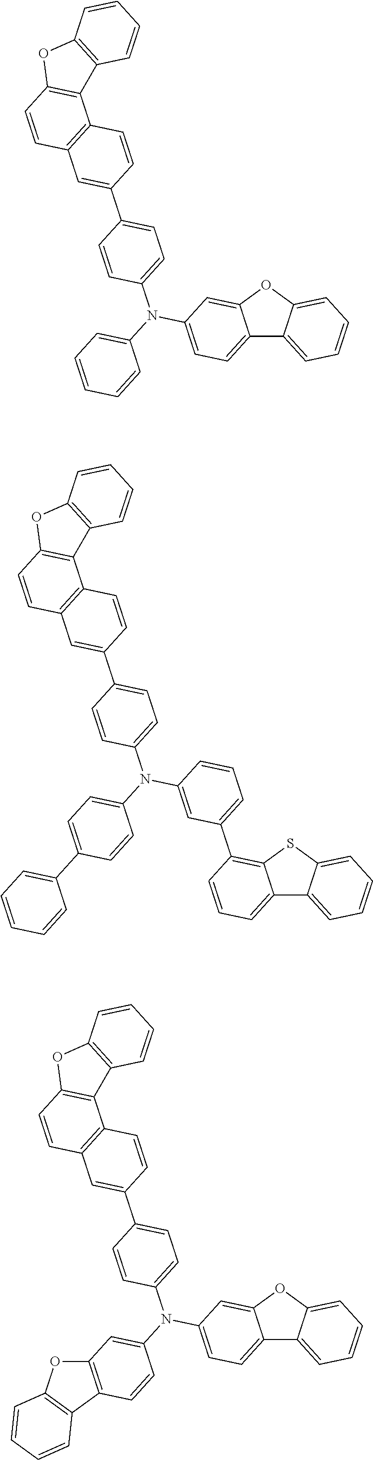 [SMILES: c1ccc(N(c2ccc(-c3ccc4c(ccc5oc6ccccc6c54)c3)cc2)c2ccc3c(c2)oc2ccccc23)cc1 ; c1ccc(-c2ccc(N(c3ccc(-c4ccc5c(ccc6oc7ccccc7c65)c4)cc3)c3cccc(-c4cccc5c4sc4ccccc45)c3)cc2)cc1 ; c1ccc2c(c1)oc1cc(N(c3ccc(-c4ccc5c(ccc6oc7ccccc7c65)c4)cc3)c3ccc4c(c3)oc3ccccc34)ccc12]

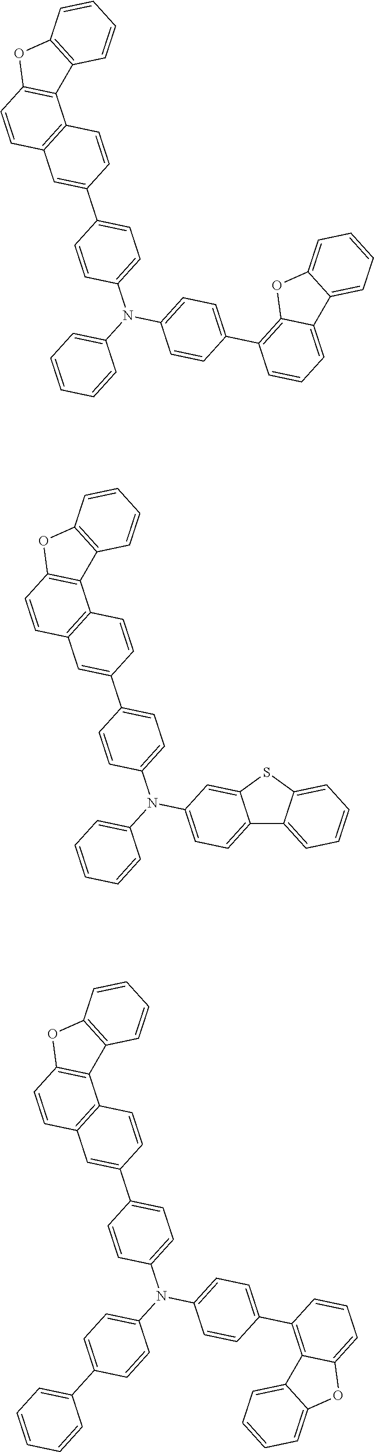 [SMILES: c1ccc(N(c2ccc(-c3ccc4c(ccc5oc6ccccc6c54)c3)cc2)c2ccc(-c3cccc4c3oc3ccccc34)cc2)cc1 ; c1ccc(N(c2ccc(-c3ccc4c(ccc5oc6ccccc6c54)c3)cc2)c2ccc3c(c2)sc2ccccc23)cc1 ; c1ccc(-c2ccc(N(c3ccc(-c4ccc5c(ccc6oc7ccccc7c65)c4)cc3)c3ccc(-c4cccc5oc6ccccc6c45)cc3)cc2)cc1]

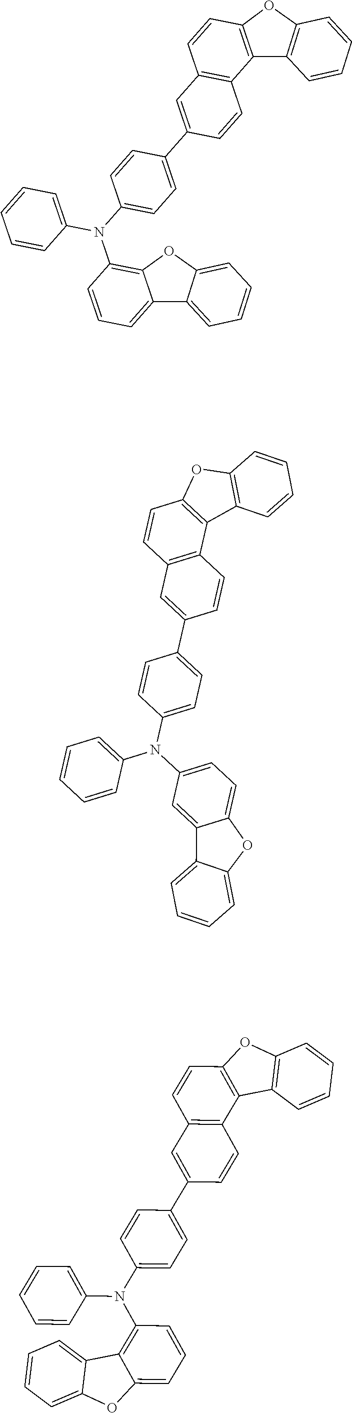 [SMILES: c1ccc(N(c2ccc(-c3ccc4c(ccc5oc6ccccc6c54)c3)cc2)c2cccc3c2oc2ccccc23)cc1 ; c1ccc(N(c2ccc(-c3ccc4c(ccc5oc6ccccc6c54)c3)cc2)c2ccc3oc4ccccc4c3c2)cc1 ; c1ccc(N(c2ccc(-c3ccc4c(ccc5oc6ccccc6c54)c3)cc2)c2cccc3oc4ccccc4c23)cc1]

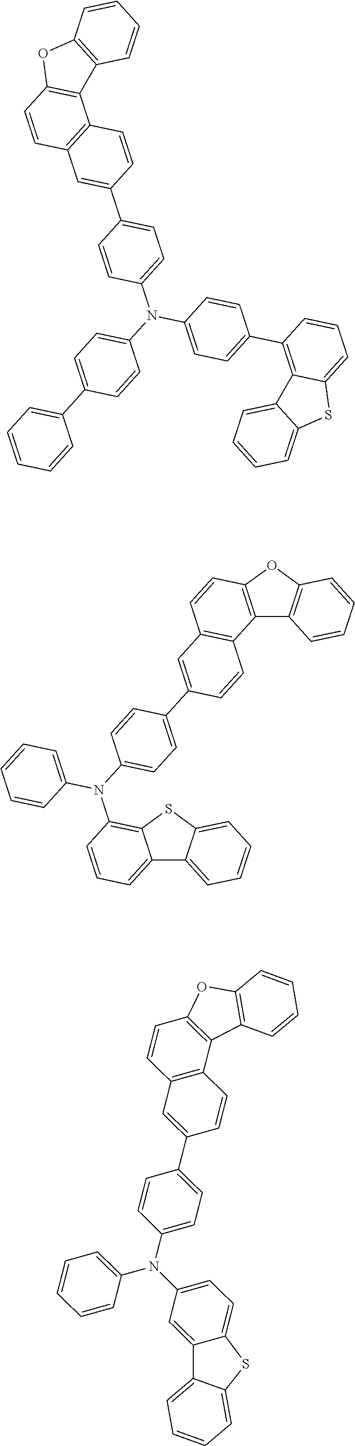 [SMILES: c1ccc(-c2ccc(N(c3ccc(-c4ccc5c(ccc6oc7ccccc7c65)c4)cc3)c3ccc(-c4cccc5sc6ccccc6c45)cc3)cc2)cc1 ; c1ccc(N(c2ccc(-c3ccc4c(ccc5oc6ccccc6c54)c3)cc2)c2cccc3c2sc2ccccc23)cc1 ; c1ccc(N(c2ccc(-c3ccc4c(ccc5oc6ccccc6c54)c3)cc2)c2ccc3sc4ccccc4c3c2)cc1]

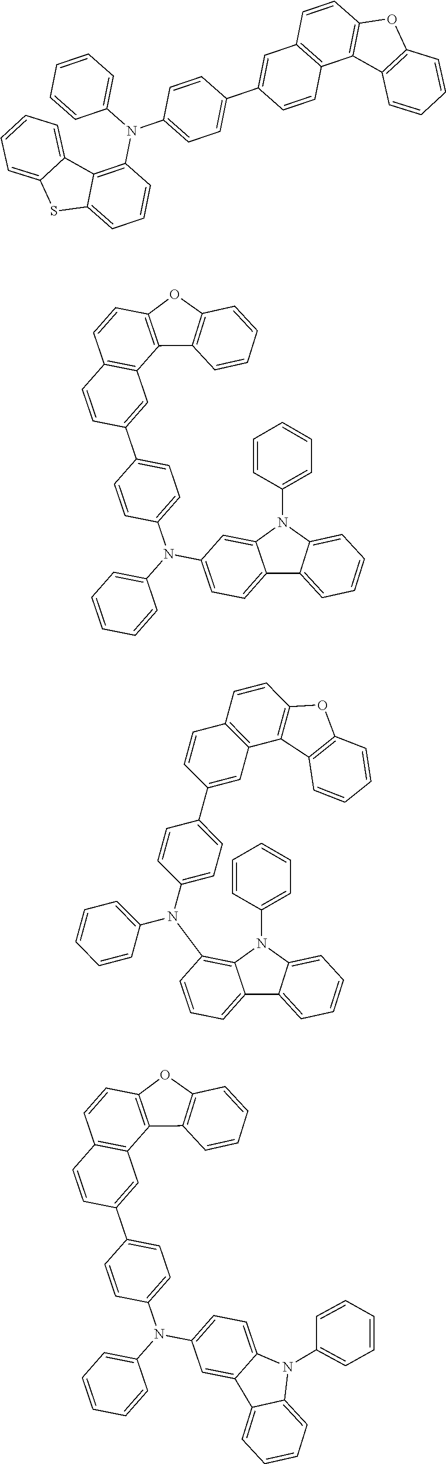 [SMILES: c1ccc(N(c2ccc(-c3ccc4c(ccc5oc6ccccc6c54)c3)cc2)c2cccc3sc4ccccc4c23)cc1 ; c1ccc(N(c2ccc(-c3ccc4ccc5oc6ccccc6c5c4c3)cc2)c2ccc3c4ccccc4n(-c4ccccc4)c3c2)cc1 ; c1ccc(N(c2ccc(-c3ccc4ccc5oc6ccccc6c5c4c3)cc2)c2cccc3c4ccccc4n(-c4ccccc4)c23)cc1 ; c1ccc(N(c2ccc(-c3ccc4ccc5oc6ccccc6c5c4c3)cc2)c2ccc3c(c2)c2ccccc2n3-c2ccccc2)cc1]

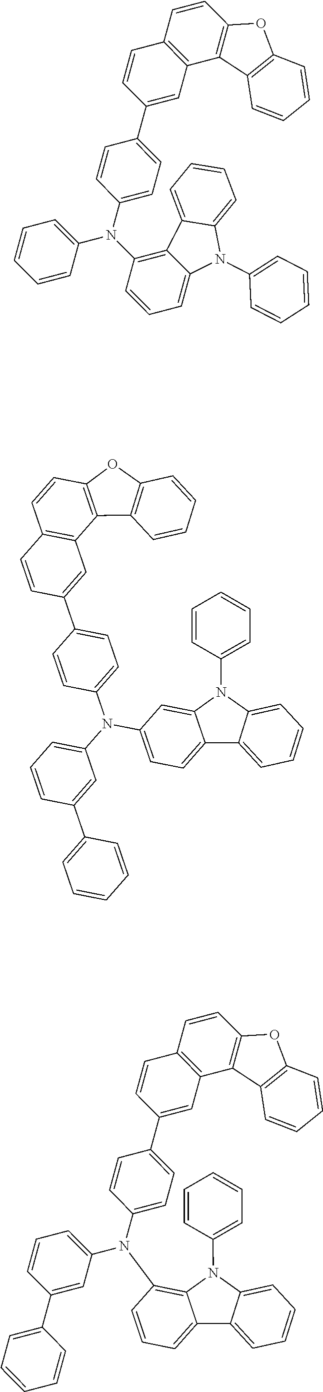 [SMILES: c1ccc(N(c2ccc(-c3ccc4ccc5oc6ccccc6c5c4c3)cc2)c2cccc3c2c2ccccc2n3-c2ccccc2)cc1 ; c1ccc(-c2cccc(N(c3ccc(-c4ccc5ccc6oc7ccccc7c6c5c4)cc3)c3ccc4c5ccccc5n(-c5ccccc5)c4c3)c2)cc1 ; c1ccc(-c2cccc(N(c3ccc(-c4ccc5ccc6oc7ccccc7c6c5c4)cc3)c3cccc4c5ccccc5n(-c5ccccc5)c34)c2)cc1]

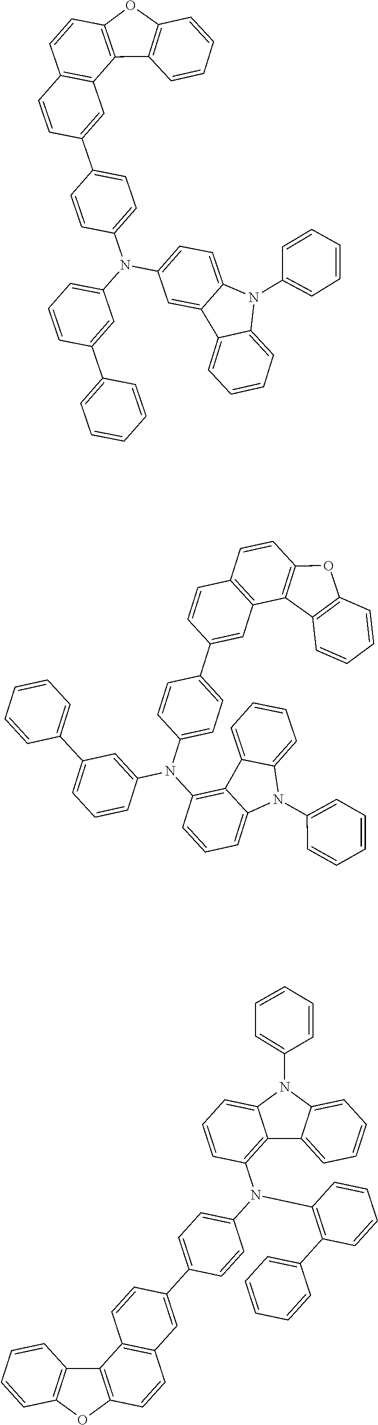 [SMILES: c1ccc(-c2cccc(N(c3ccc(-c4ccc5ccc6oc7ccccc7c6c5c4)cc3)c3ccc4c(c3)c3ccccc3n4-c3ccccc3)c2)cc1 ; c1ccc(-c2cccc(N(c3ccc(-c4ccc5ccc6oc7ccccc7c6c5c4)cc3)c3cccc4c3c3ccccc3n4-c3ccccc3)c2)cc1 ; c1ccc(-c2ccccc2N(c2ccc(-c3ccc4c(ccc5oc6ccccc6c54)c3)cc2)c2cccc3c2c2ccccc2n3-c2ccccc2)cc1]

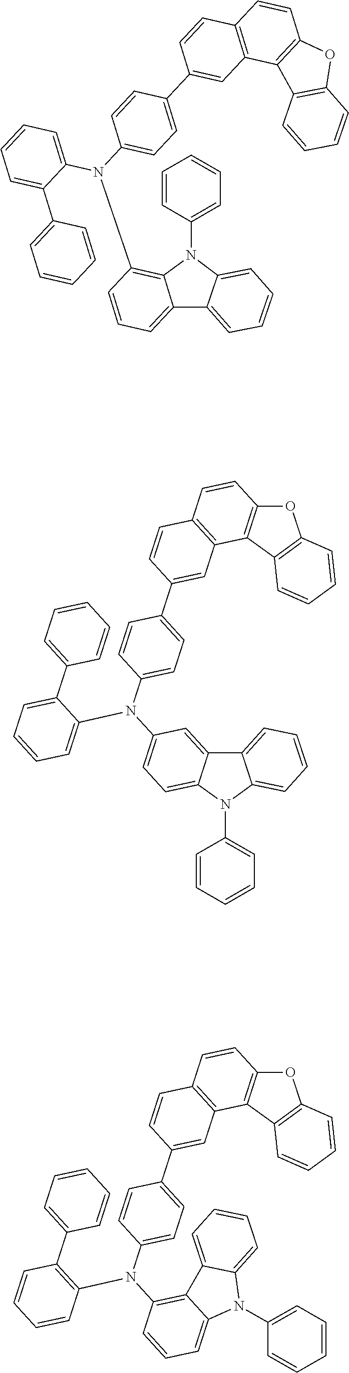 [SMILES: c1ccc(-c2ccccc2N(c2ccc(-c3ccc4ccc5oc6ccccc6c5c4c3)cc2)c2cccc3c4ccccc4n(-c4ccccc4)c23)cc1 ; c1ccc(-c2ccccc2N(c2ccc(-c3ccc4ccc5oc6ccccc6c5c4c3)cc2)c2ccc3c(c2)c2ccccc2n3-c2ccccc2)cc1 ; c1ccc(-c2ccccc2N(c2ccc(-c3ccc4ccc5oc6ccccc6c5c4c3)cc2)c2cccc3c2c2ccccc2n3-c2ccccc2)cc1]

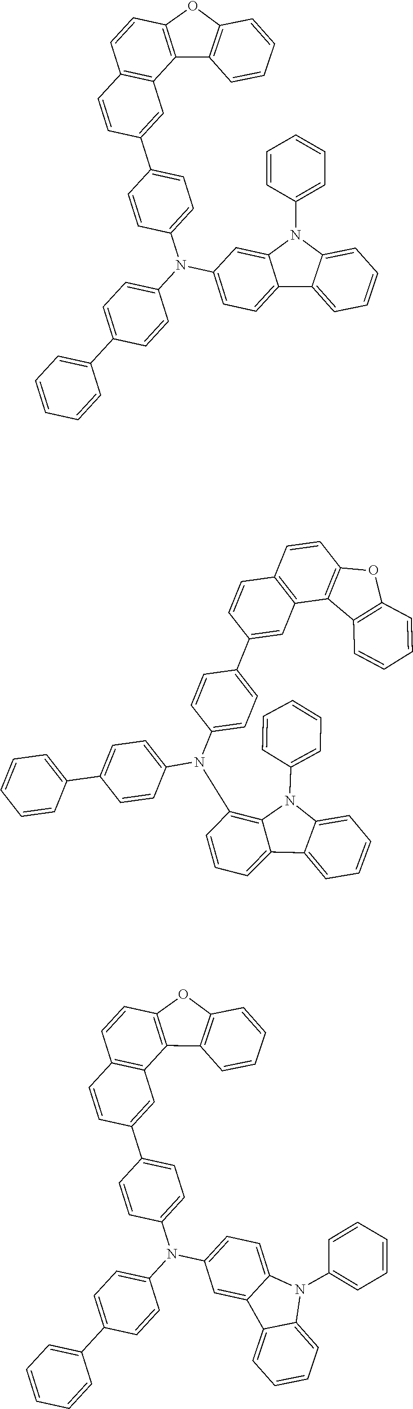 [SMILES: c1ccc(-c2ccc(N(c3ccc(-c4ccc5ccc6oc7ccccc7c6c5c4)cc3)c3ccc4c5ccccc5n(-c5ccccc5)c4c3)cc2)cc1 ; c1ccc(-c2ccc(N(c3ccc(-c4ccc5ccc6oc7ccccc7c6c5c4)cc3)c3cccc4c5ccccc5n(-c5ccccc5)c34)cc2)cc1 ; c1ccc(-c2ccc(N(c3ccc(-c4ccc5ccc6oc7ccccc7c6c5c4)cc3)c3ccc4c(c3)c3ccccc3n4-c3ccccc3)cc2)cc1]

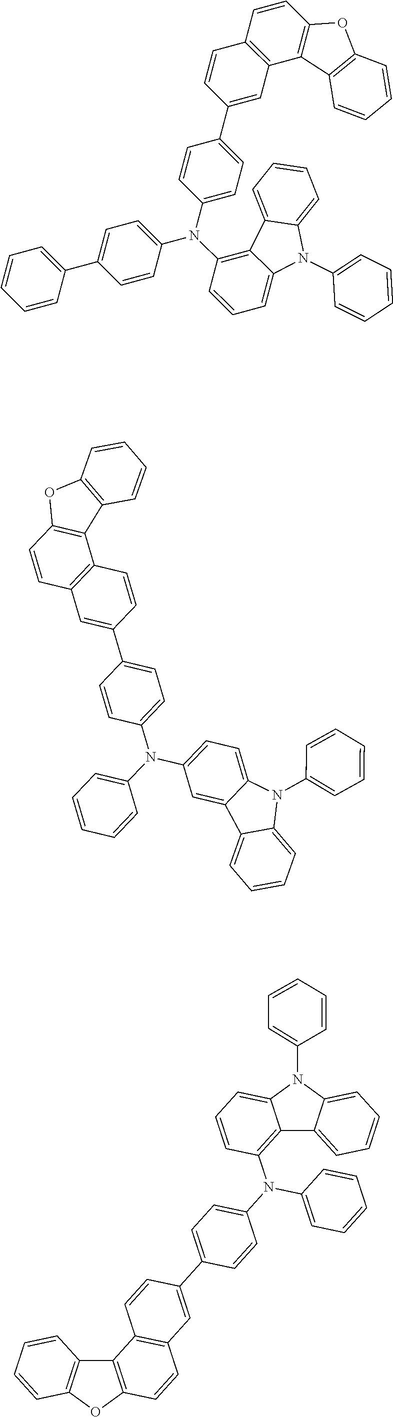 [SMILES: c1ccc(-c2ccc(N(c3ccc(-c4ccc5ccc6oc7ccccc7c6c5c4)cc3)c3cccc4c3c3ccccc3n4-c3ccccc3)cc2)cc1 ; c1ccc(N(c2ccc(-c3ccc4c(ccc5oc6ccccc6c54)c3)cc2)c2ccc3c(c2)c2ccccc2n3-c2ccccc2)cc1 ; c1ccc(N(c2ccc(-c3ccc4c(ccc5oc6ccccc6c54)c3)cc2)c2cccc3c2c2ccccc2n3-c2ccccc2)cc1]

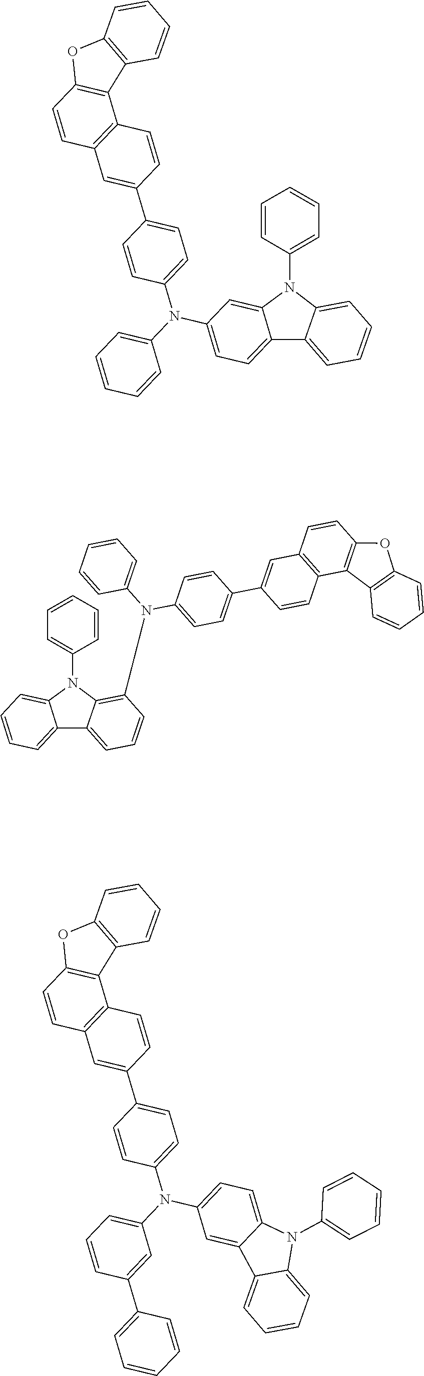 [SMILES: c1ccc(N(c2ccc(-c3ccc4c(ccc5oc6ccccc6c54)c3)cc2)c2ccc3c4ccccc4n(-c4ccccc4)c3c2)cc1 ; c1ccc(N(c2ccc(-c3ccc4c(ccc5oc6ccccc6c54)c3)cc2)c2cccc3c4ccccc4n(-c4ccccc4)c23)cc1 ; c1ccc(-c2cccc(N(c3ccc(-c4ccc5c(ccc6oc7ccccc7c65)c4)cc3)c3ccc4c(c3)c3ccccc3n4-c3ccccc3)c2)cc1]

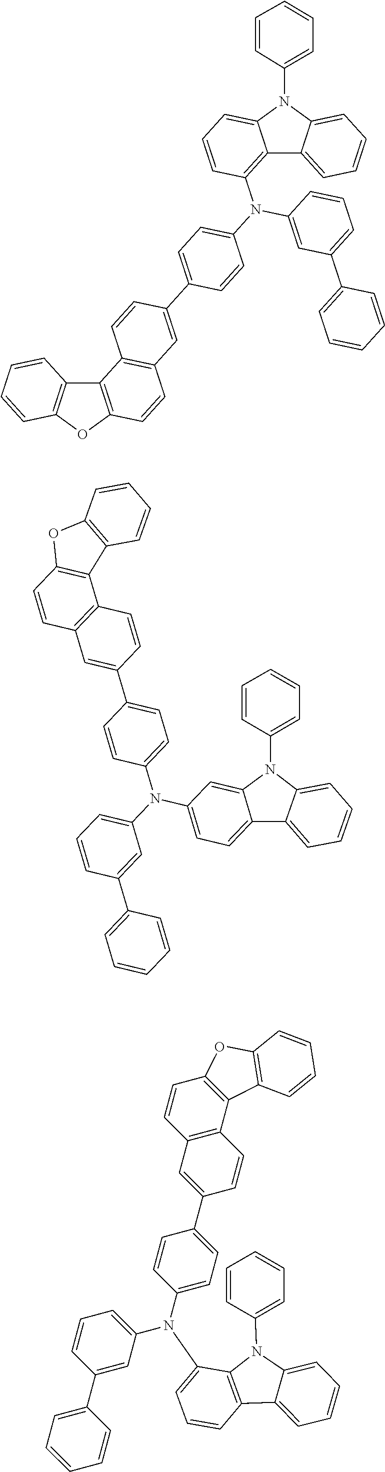 [SMILES: c1ccc(-c2cccc(N(c3ccc(-c4ccc5c(ccc6oc7ccccc7c65)c4)cc3)c3cccc4c3c3ccccc3n4-c3ccccc3)c2)cc1 ; c1ccc(-c2cccc(N(c3ccc(-c4ccc5c(ccc6oc7ccccc7c65)c4)cc3)c3ccc4c5ccccc5n(-c5ccccc5)c4c3)c2)cc1 ; c1ccc(-c2cccc(N(c3ccc(-c4ccc5c(ccc6oc7ccccc7c65)c4)cc3)c3cccc4c5ccccc5n(-c5ccccc5)c34)c2)cc1]

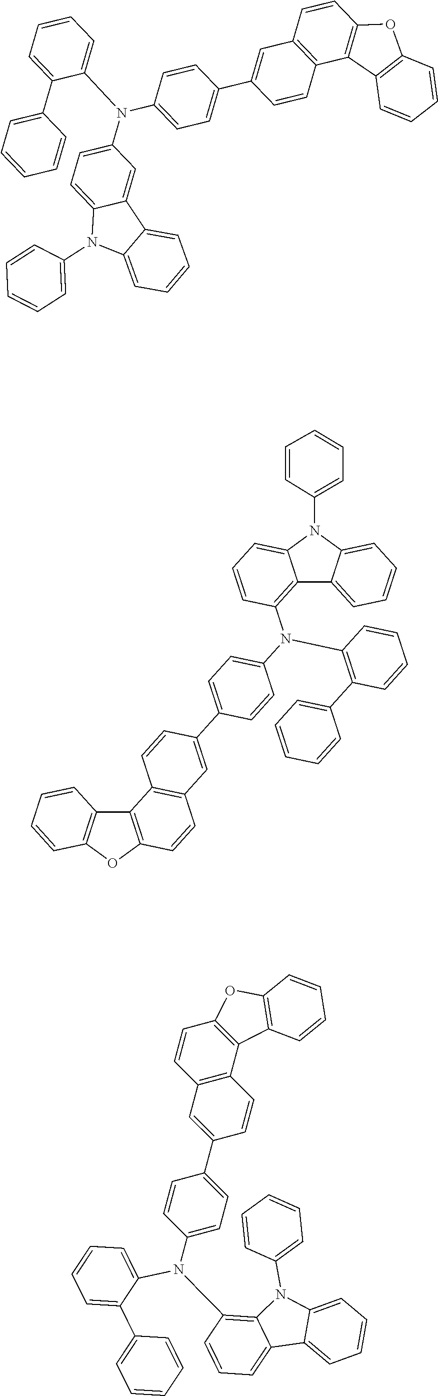 [SMILES: c1ccc(-c2ccccc2N(c2ccc(-c3ccc4c(ccc5oc6ccccc6c54)c3)cc2)c2ccc3c(c2)c2ccccc2n3-c2ccccc2)cc1 ; c1ccc(-c2ccccc2N(c2ccc(-c3ccc4c(ccc5oc6ccccc6c54)c3)cc2)c2cccc3c2c2ccccc2n3-c2ccccc2)cc1 ; c1ccc(-c2ccccc2N(c2ccc(-c3ccc4c(ccc5oc6ccccc6c54)c3)cc2)c2cccc3c4ccccc4n(-c4ccccc4)c23)cc1]

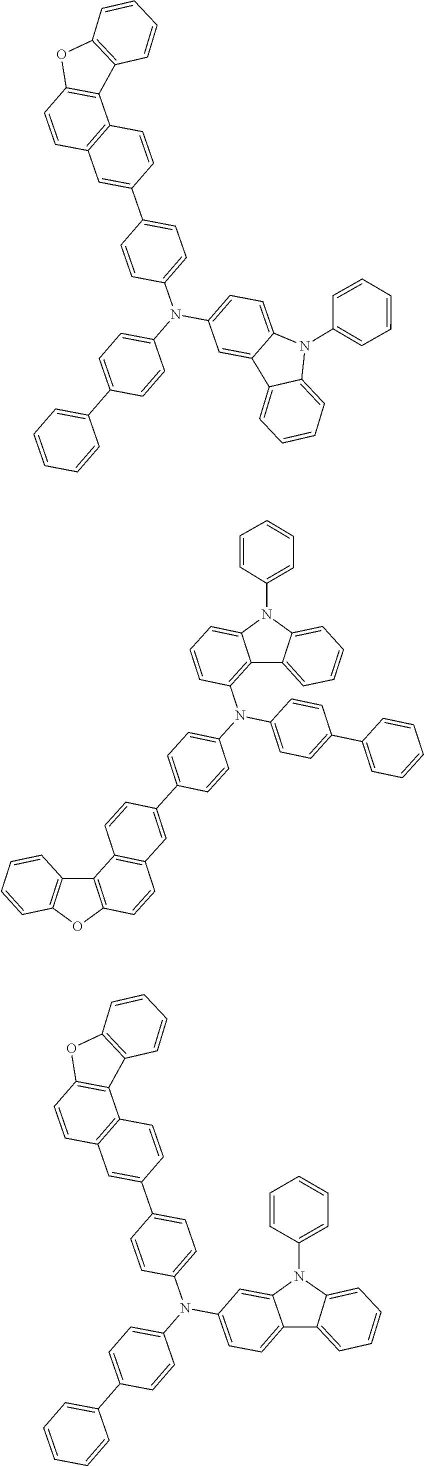 [SMILES: c1ccc(-c2ccc(N(c3ccc(-c4ccc5c(ccc6oc7ccccc7c65)c4)cc3)c3ccc4c(c3)c3ccccc3n4-c3ccccc3)cc2)cc1 ; c1ccc(-c2ccc(N(c3ccc(-c4ccc5c(ccc6oc7ccccc7c65)c4)cc3)c3cccc4c3c3ccccc3n4-c3ccccc3)cc2)cc1 ; c1ccc(-c2ccc(N(c3ccc(-c4ccc5c(ccc6oc7ccccc7c65)c4)cc3)c3ccc4c5ccccc5n(-c5ccccc5)c4c3)cc2)cc1]

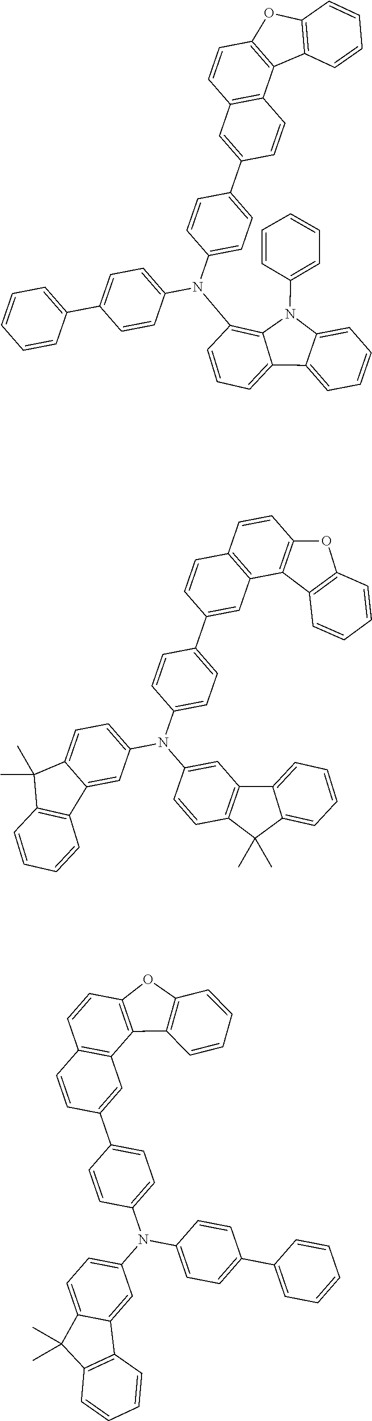 [SMILES: c1ccc(-c2ccc(N(c3ccc(-c4ccc5c(ccc6oc7ccccc7c65)c4)cc3)c3cccc4c5ccccc5n(-c5ccccc5)c34)cc2)cc1 ; CC1(C)c2ccccc2-c2cc(N(c3ccc(-c4ccc5ccc6oc7ccccc7c6c5c4)cc3)c3ccc4c(c3)-c3ccccc3C4(C)C)ccc21 ; CC1(C)c2ccccc2-c2cc(N(c3ccc(-c4ccccc4)cc3)c3ccc(-c4ccc5ccc6oc7ccccc7c6c5c4)cc3)ccc21]

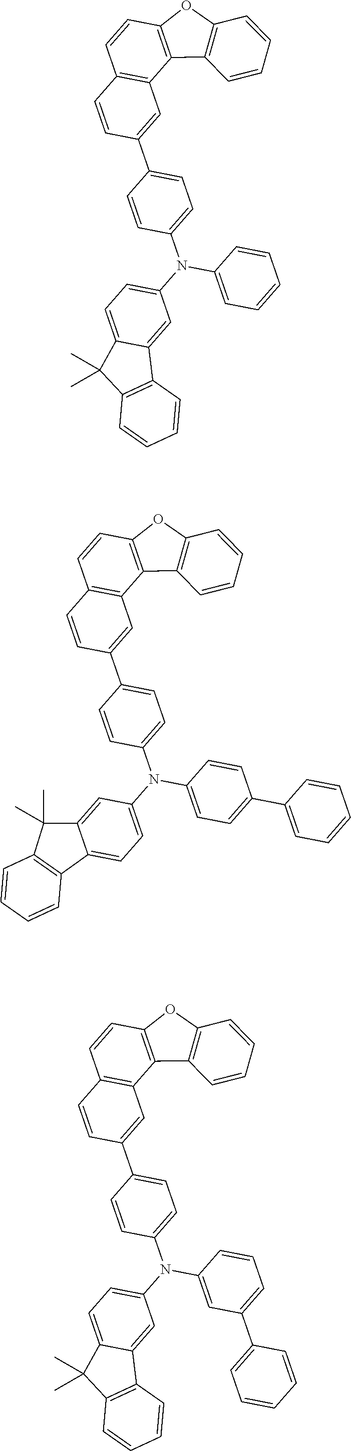 [SMILES: CC1(C)c2ccccc2-c2cc(N(c3ccccc3)c3ccc(-c4ccc5ccc6oc7ccccc7c6c5c4)cc3)ccc21 ; CC1(C)c2ccccc2-c2ccc(N(c3ccc(-c4ccccc4)cc3)c3ccc(-c4ccc5ccc6oc7ccccc7c6c5c4)cc3)cc21 ; CC1(C)c2ccccc2-c2cc(N(c3ccc(-c4ccc5ccc6oc7ccccc7c6c5c4)cc3)c3cccc(-c4ccccc4)c3)ccc21]

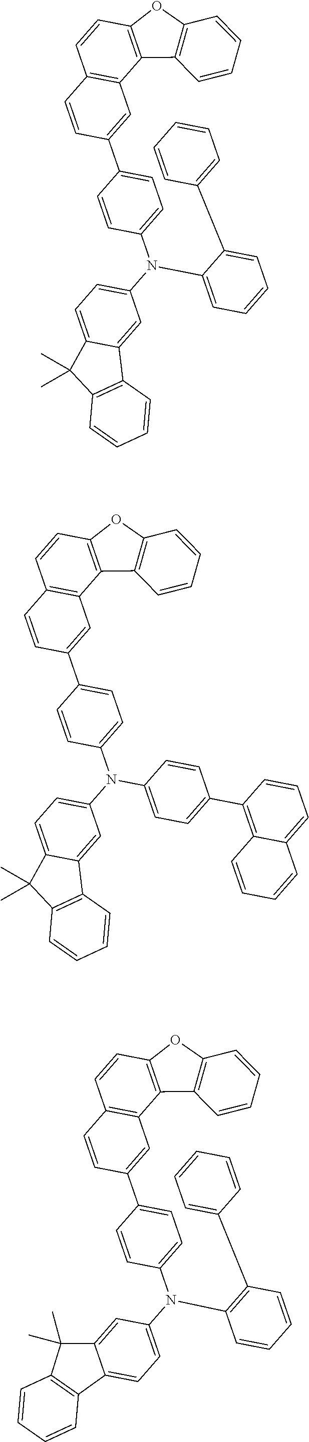 [SMILES: CC1(C)c2ccccc2-c2cc(N(c3ccc(-c4ccc5ccc6oc7ccccc7c6c5c4)cc3)c3ccccc3-c3ccccc3)ccc21 ; CC1(C)c2ccccc2-c2cc(N(c3ccc(-c4ccc5ccc6oc7ccccc7c6c5c4)cc3)c3ccc(-c4cccc5ccccc45)cc3)ccc21 ; CC1(C)c2ccccc2-c2ccc(N(c3ccc(-c4ccc5ccc6oc7ccccc7c6c5c4)cc3)c3ccccc3-c3ccccc3)cc21]

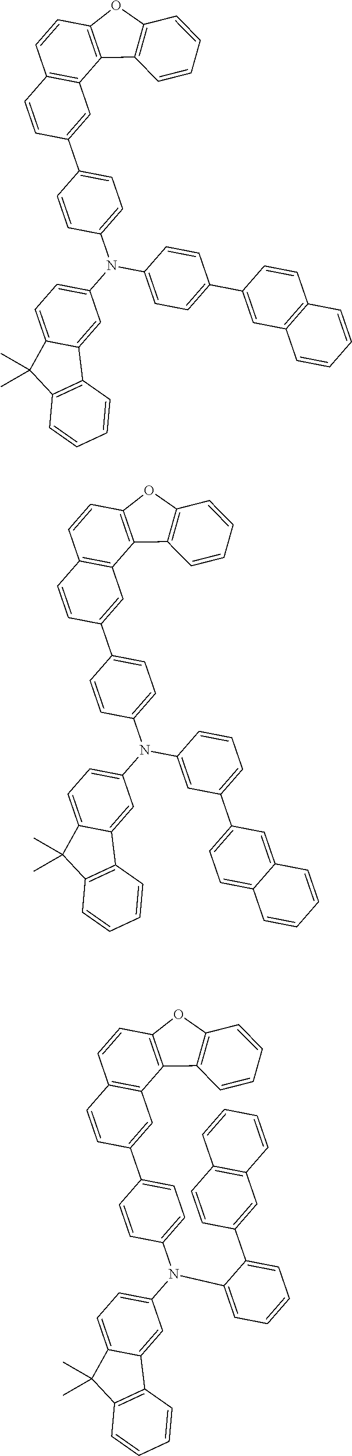 [SMILES: CC1(C)c2ccccc2-c2cc(N(c3ccc(-c4ccc5ccccc5c4)cc3)c3ccc(-c4ccc5ccc6oc7ccccc7c6c5c4)cc3)ccc21 ; CC1(C)c2ccccc2-c2cc(N(c3ccc(-c4ccc5ccc6oc7ccccc7c6c5c4)cc3)c3cccc(-c4ccc5ccccc5c4)c3)ccc21 ; CC1(C)c2ccccc2-c2cc(N(c3ccc(-c4ccc5ccc6oc7ccccc7c6c5c4)cc3)c3ccccc3-c3ccc4ccccc4c3)ccc21]

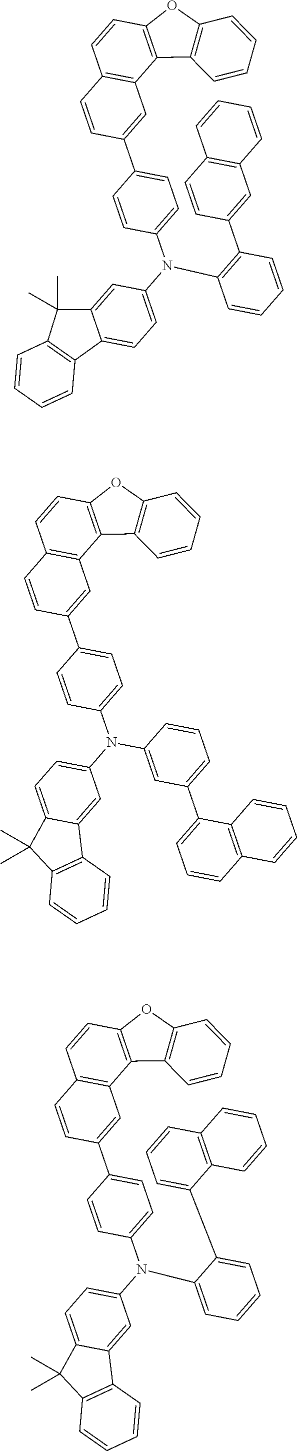 [SMILES: CC1(C)c2ccccc2-c2ccc(N(c3ccc(-c4ccc5ccc6oc7ccccc7c6c5c4)cc3)c3ccccc3-c3ccc4ccccc4c3)cc21 ; CC1(C)c2ccccc2-c2cc(N(c3ccc(-c4ccc5ccc6oc7ccccc7c6c5c4)cc3)c3cccc(-c4cccc5ccccc45)c3)ccc21 ; CC1(C)c2ccccc2-c2cc(N(c3ccc(-c4ccc5ccc6oc7ccccc7c6c5c4)cc3)c3ccccc3-c3cccc4ccccc34)ccc21]

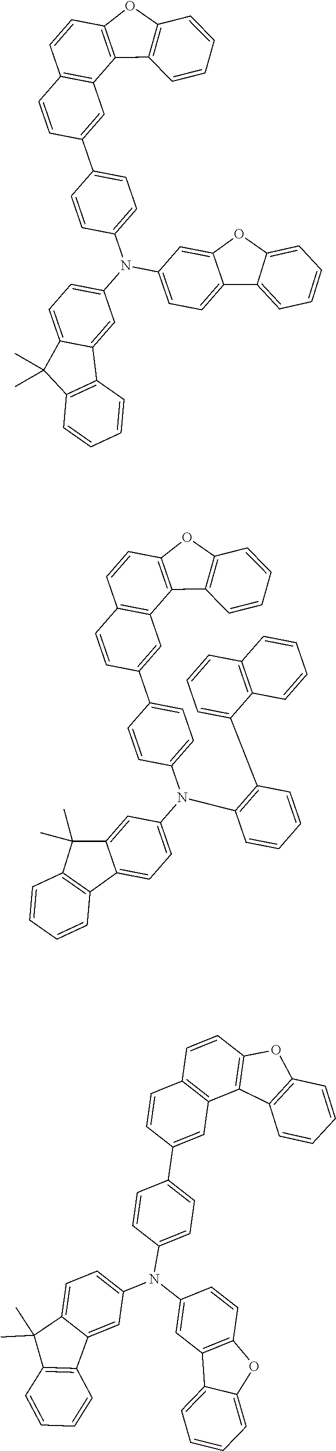 [SMILES: CC1(C)c2ccccc2-c2cc(N(c3ccc(-c4ccc5ccc6oc7ccccc7c6c5c4)cc3)c3ccc4c(c3)oc3ccccc34)ccc21 ; CC1(C)c2ccccc2-c2ccc(N(c3ccc(-c4ccc5ccc6oc7ccccc7c6c5c4)cc3)c3ccccc3-c3cccc4ccccc34)cc21 ; CC1(C)c2ccccc2-c2cc(N(c3ccc(-c4ccc5ccc6oc7ccccc7c6c5c4)cc3)c3ccc4oc5ccccc5c4c3)ccc21]

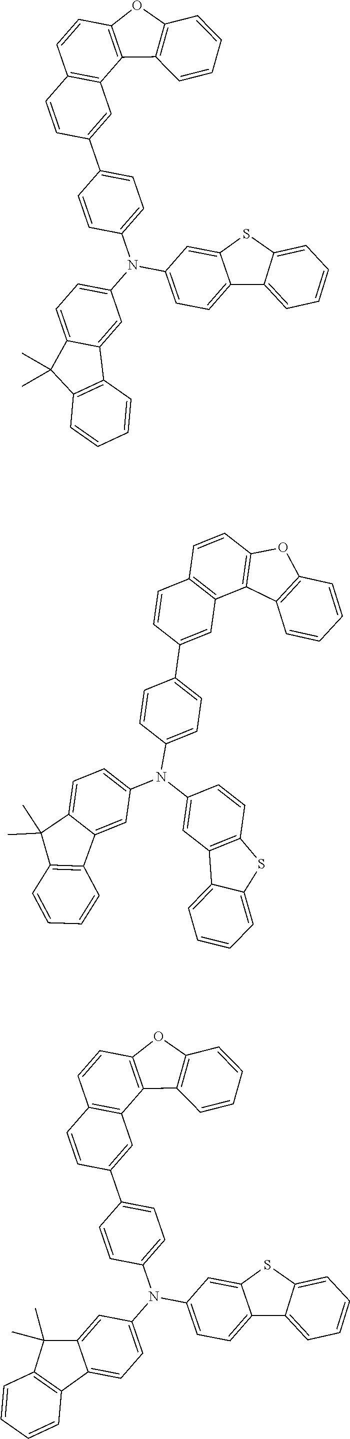 [SMILES: CC1(C)c2ccccc2-c2cc(N(c3ccc(-c4ccc5ccc6oc7ccccc7c6c5c4)cc3)c3ccc4c(c3)sc3ccccc34)ccc21 ; CC1(C)c2ccccc2-c2cc(N(c3ccc(-c4ccc5ccc6oc7ccccc7c6c5c4)cc3)c3ccc4sc5ccccc5c4c3)ccc21 ; CC1(C)c2ccccc2-c2ccc(N(c3ccc(-c4ccc5ccc6oc7ccccc7c6c5c4)cc3)c3ccc4c(c3)sc3ccccc34)cc21]

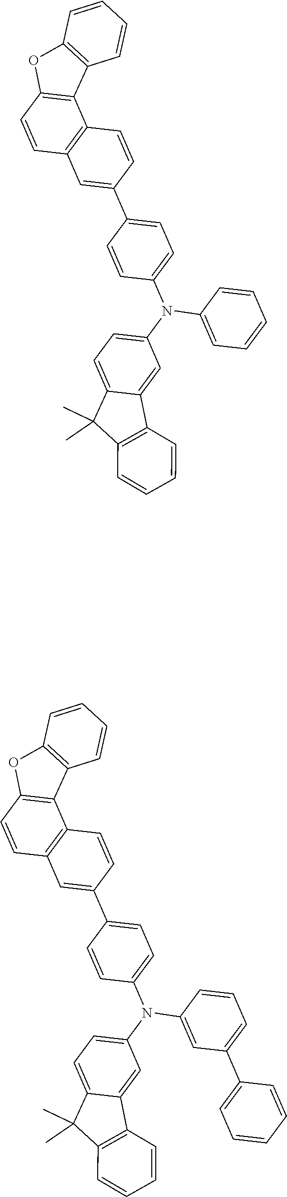 [SMILES: CC1(C)c2ccccc2-c2cc(N(c3ccccc3)c3ccc(-c4ccc5c(ccc6oc7ccccc7c65)c4)cc3)ccc21 ; CC1(C)c2ccccc2-c2cc(N(c3ccc(-c4ccc5c(ccc6oc7ccccc7c65)c4)cc3)c3cccc(-c4ccccc4)c3)ccc21]

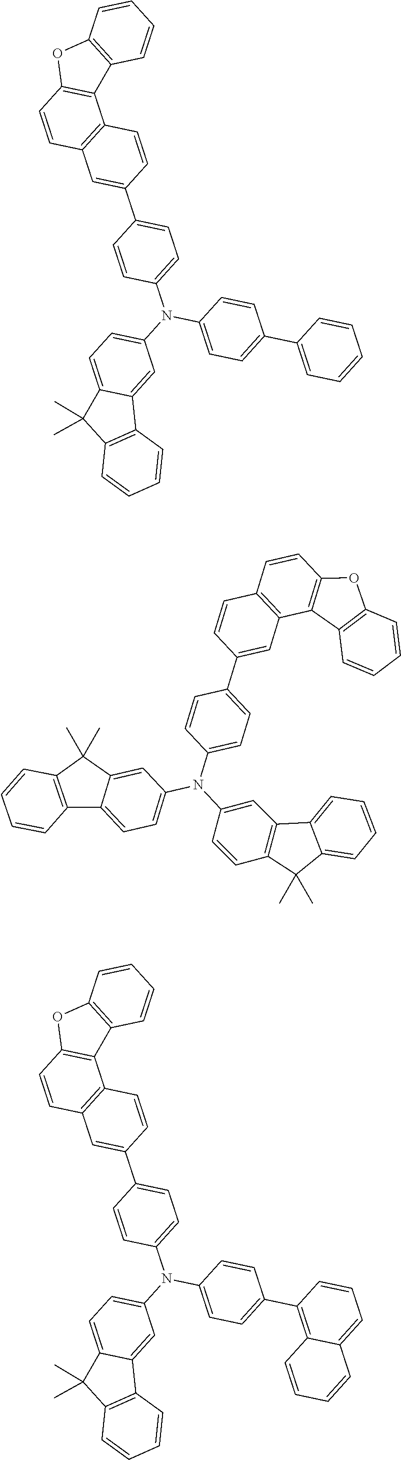 [SMILES: CC1(C)c2ccccc2-c2cc(N(c3ccc(-c4ccccc4)cc3)c3ccc(-c4ccc5c(ccc6oc7ccccc7c65)c4)cc3)ccc21 ; CC1(C)c2ccccc2-c2cc(N(c3ccc(-c4ccc5ccc6oc7ccccc7c6c5c4)cc3)c3ccc4c(c3)C(C)(C)c3ccccc3-4)ccc21 ; CC1(C)c2ccccc2-c2cc(N(c3ccc(-c4ccc5c(ccc6oc7ccccc7c65)c4)cc3)c3ccc(-c4cccc5ccccc45)cc3)ccc21]

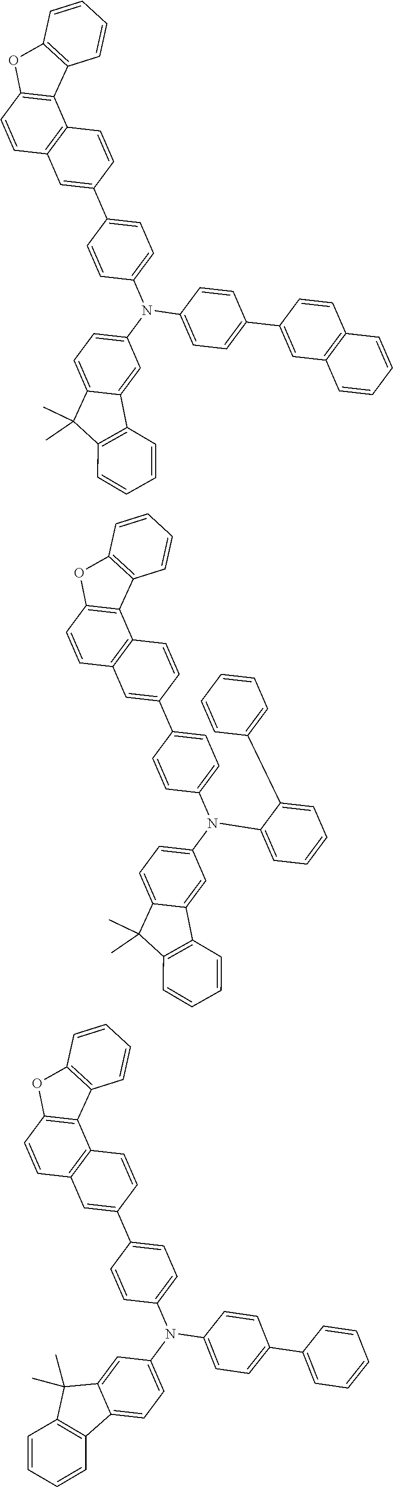

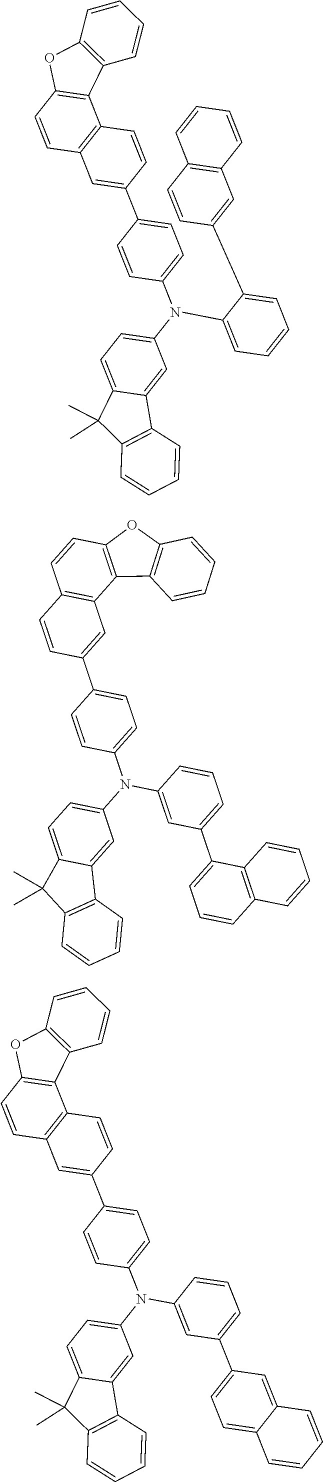

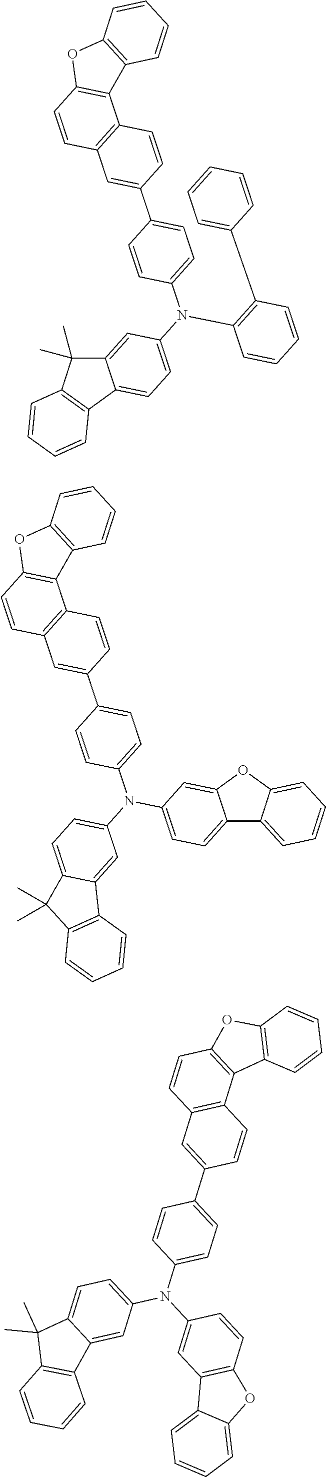

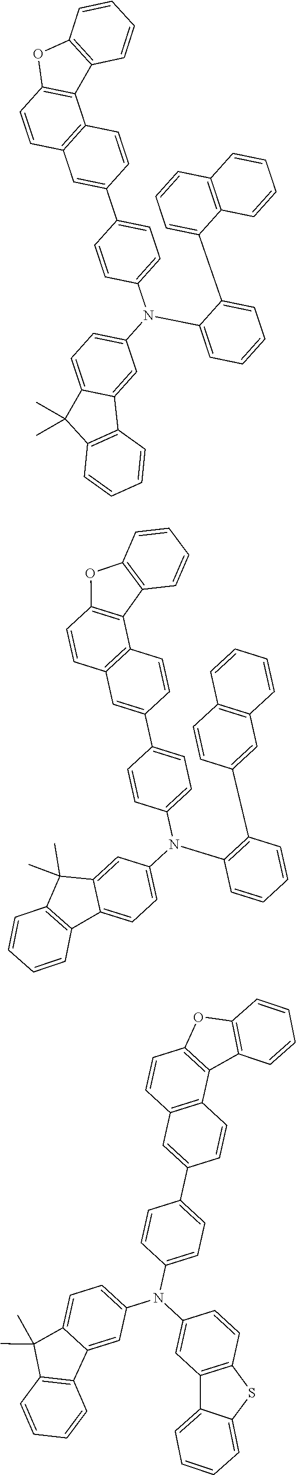

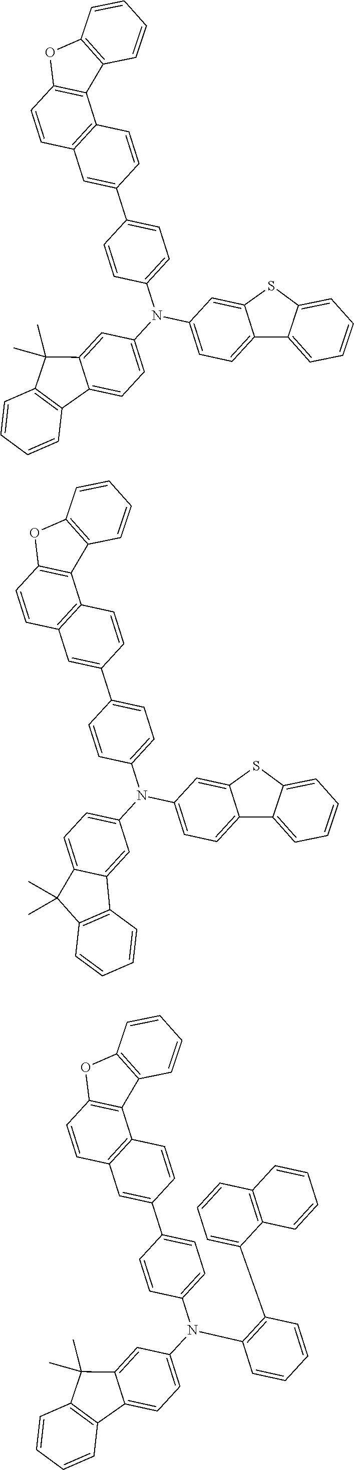 [SMILES: CC1(C)c2ccccc2-c2ccc(N(c3ccc(-c4ccc5c(ccc6oc7ccccc7c65)c4)cc3)c3ccc4c(c3)sc3ccccc34)cc21 ; CC1(C)c2ccccc2-c2cc(N(c3ccc(-c4ccc5c(ccc6oc7ccccc7c65)c4)cc3)c3ccc4c(c3)sc3ccccc34)ccc21 ; CC1(C)c2ccccc2-c2ccc(N(c3ccc(-c4ccc5c(ccc6oc7ccccc7c65)c4)cc3)c3ccccc3-c3cccc4ccccc34)cc21]

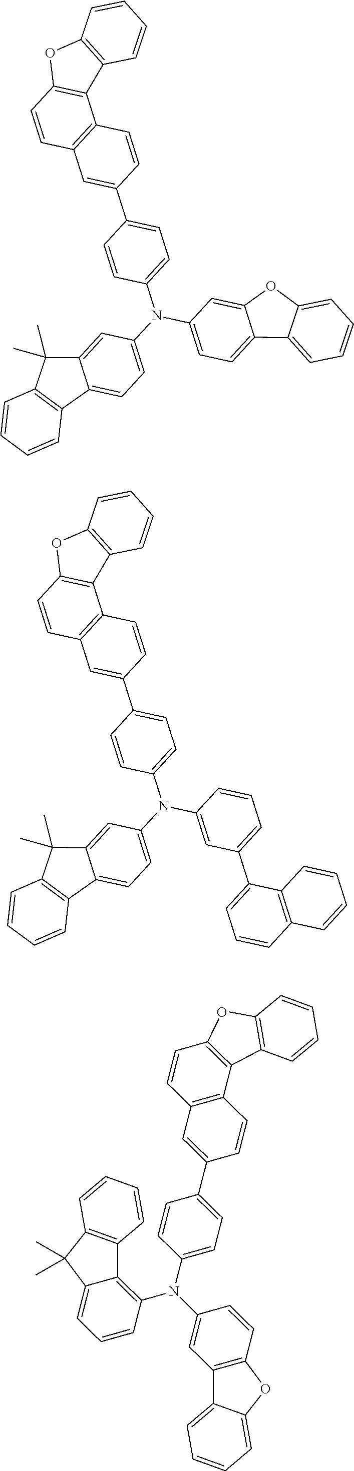 [SMILES: CC1(C)c2ccccc2-c2ccc(N(c3ccc(-c4ccc5c(ccc6oc7ccccc7c65)c4)cc3)c3ccc4c(c3)oc3ccccc34)cc21 ; CC1(C)c2ccccc2-c2ccc(N(c3ccc(-c4ccc5c(ccc6oc7ccccc7c65)c4)cc3)c3cccc(-c4cccc5ccccc45)c3)cc21 ; CC1(C)c2ccccc2-c2c(N(c3ccc(-c4ccc5c(ccc6oc7ccccc7c65)c4)cc3)c3ccc4oc5ccccc5c4c3)cccc21]

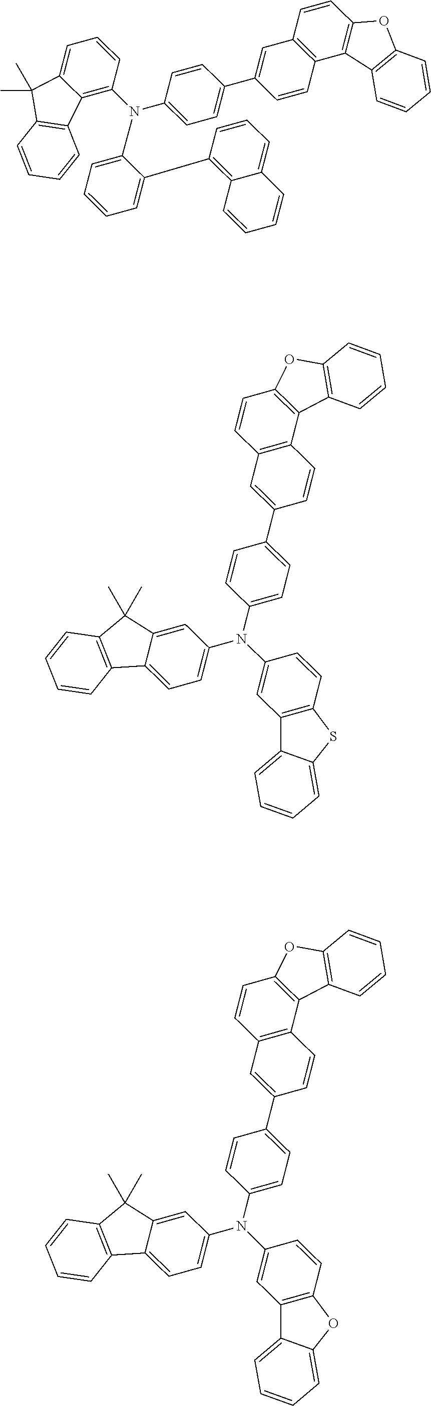 [SMILES: CC1(C)c2ccccc2-c2c(N(c3ccc(-c4ccc5c(ccc6oc7ccccc7c65)c4)cc3)c3ccccc3-c3cccc4ccccc34)cccc21 ; CC1(C)c2ccccc2-c2ccc(N(c3ccc(-c4ccc5c(ccc6oc7ccccc7c65)c4)cc3)c3ccc4sc5ccccc5c4c3)cc21 ; CC1(C)c2ccccc2-c2ccc(N(c3ccc(-c4ccc5c(ccc6oc7ccccc7c65)c4)cc3)c3ccc4oc5ccccc5c4c3)cc21]

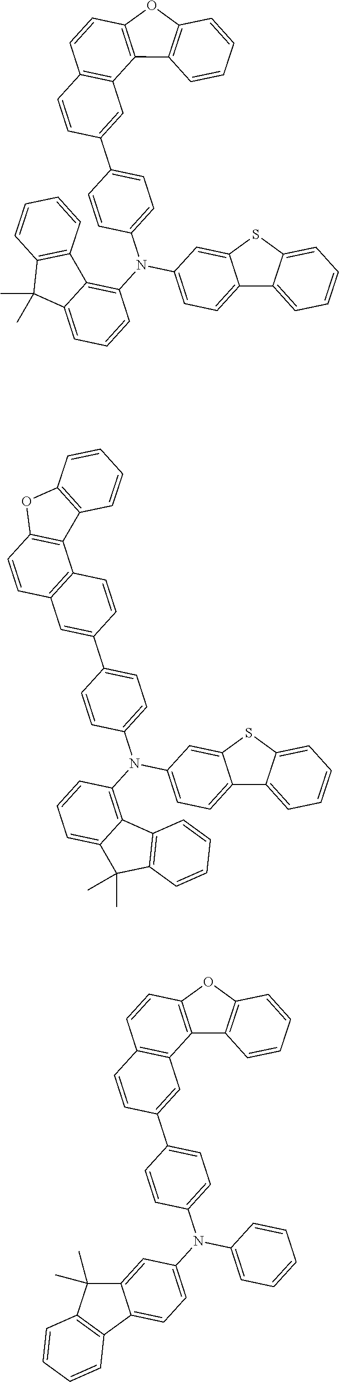 [SMILES: CC1(C)c2ccccc2-c2c(N(c3ccc(-c4ccc5ccc6oc7ccccc7c6c5c4)cc3)c3ccc4c(c3)sc3ccccc34)cccc21 ; CC1(C)c2ccccc2-c2c(N(c3ccc(-c4ccc5c(ccc6oc7ccccc7c65)c4)cc3)c3ccc4c(c3)sc3ccccc34)cccc21 ; CC1(C)c2ccccc2-c2ccc(N(c3ccccc3)c3ccc(-c4ccc5ccc6oc7ccccc7c6c5c4)cc3)cc21]

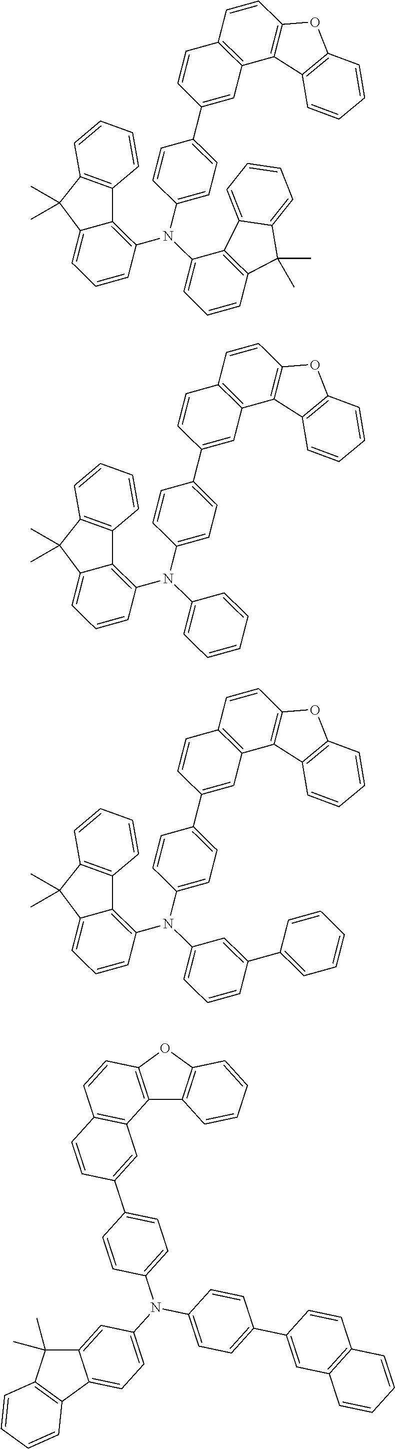

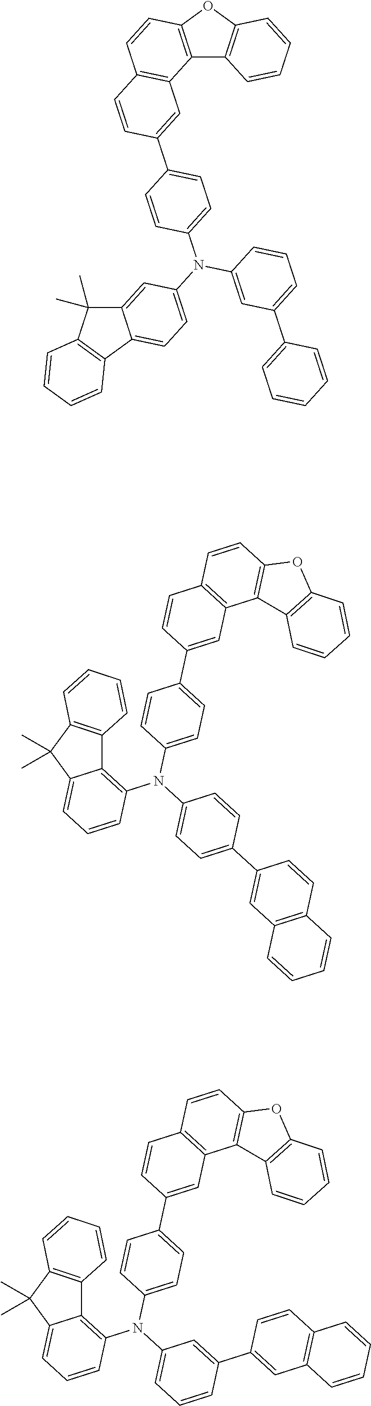 [SMILES: CC1(C)c2ccccc2-c2ccc(N(c3ccc(-c4ccc5ccc6oc7ccccc7c6c5c4)cc3)c3cccc(-c4ccccc4)c3)cc21 ; CC1(C)c2ccccc2-c2c(N(c3ccc(-c4ccc5ccccc5c4)cc3)c3ccc(-c4ccc5ccc6oc7ccccc7c6c5c4)cc3)cccc21 ; CC1(C)c2ccccc2-c2c(N(c3ccc(-c4ccc5ccc6oc7ccccc7c6c5c4)cc3)c3cccc(-c4ccc5ccccc5c4)c3)cccc21]

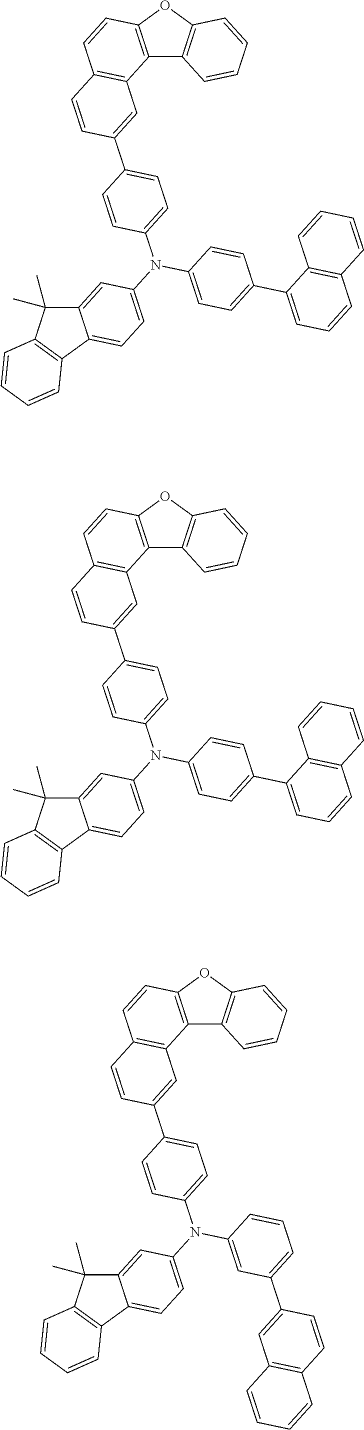 [SMILES: CC1(C)c2ccccc2-c2ccc(N(c3ccc(-c4ccc5ccc6oc7ccccc7c6c5c4)cc3)c3ccc(-c4cccc5ccccc45)cc3)cc21 ; CC1(C)c2ccccc2-c2ccc(N(c3ccc(-c4ccc5ccc6oc7ccccc7c6c5c4)cc3)c3ccc(-c4cccc5ccccc45)cc3)cc21 ; CC1(C)c2ccccc2-c2ccc(N(c3ccc(-c4ccc5ccc6oc7ccccc7c6c5c4)cc3)c3cccc(-c4ccc5ccccc5c4)c3)cc21]

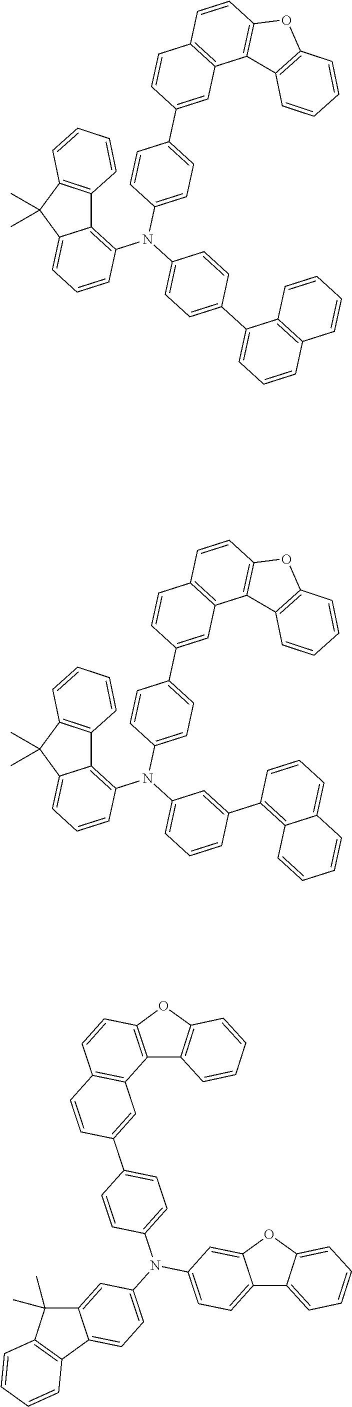 [SMILES: CC1(C)c2ccccc2-c2c(N(c3ccc(-c4ccc5ccc6oc7ccccc7c6c5c4)cc3)c3ccc(-c4cccc5ccccc45)cc3)cccc21 ; CC1(C)c2ccccc2-c2c(N(c3ccc(-c4ccc5ccc6oc7ccccc7c6c5c4)cc3)c3cccc(-c4cccc5ccccc45)c3)cccc21 ; CC1(C)c2ccccc2-c2ccc(N(c3ccc(-c4ccc5ccc6oc7ccccc7c6c5c4)cc3)c3ccc4c(c3)oc3ccccc34)cc21]

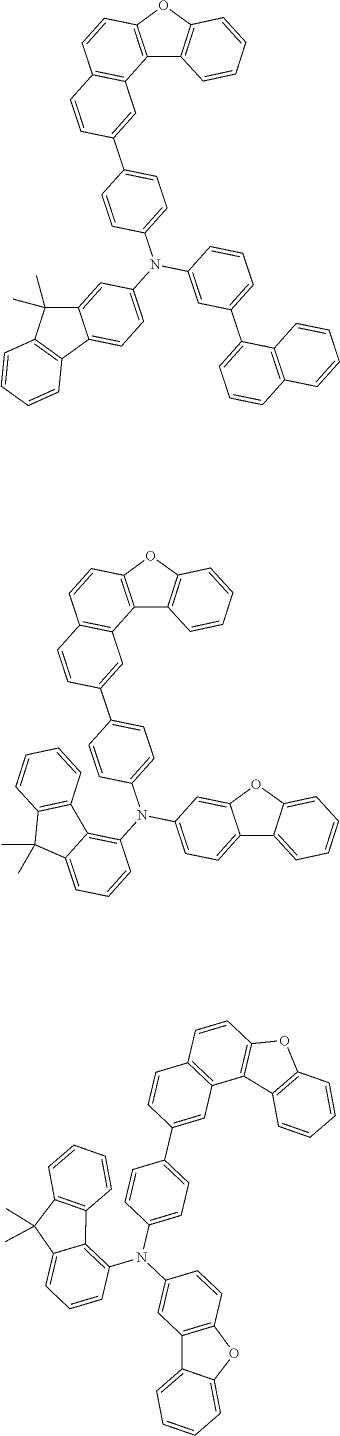 [SMILES: CC1(C)c2ccccc2-c2ccc(N(c3ccc(-c4ccc5ccc6oc7ccccc7c6c5c4)cc3)c3cccc(-c4cccc5ccccc45)c3)cc21 ; CC1(C)c2ccccc2-c2c(N(c3ccc(-c4ccc5ccc6oc7ccccc7c6c5c4)cc3)c3ccc4c(c3)oc3ccccc34)cccc21 ; CC1(C)c2ccccc2-c2c(N(c3ccc(-c4ccc5ccc6oc7ccccc7c6c5c4)cc3)c3ccc4oc5ccccc5c4c3)cccc21]

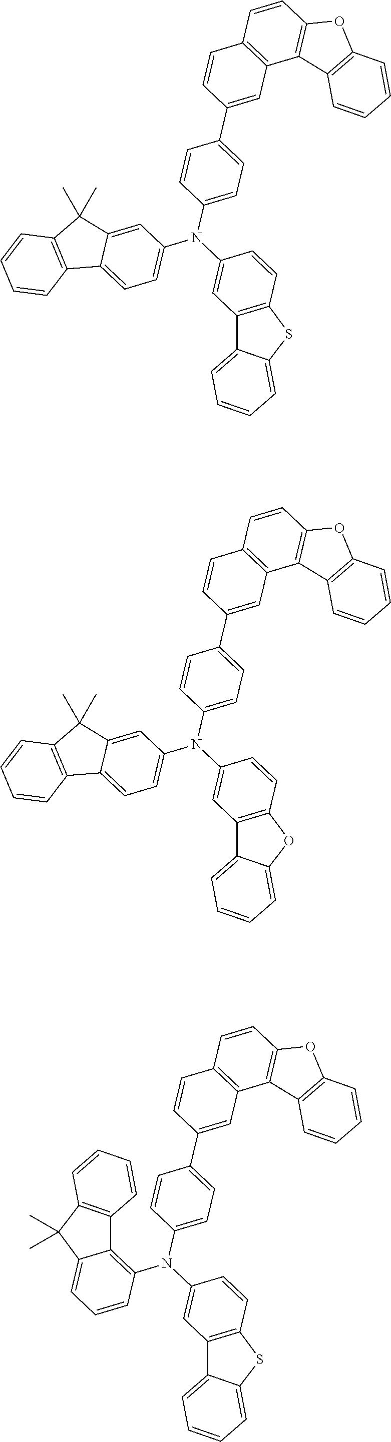 [SMILES: CC1(C)c2ccccc2-c2ccc(N(c3ccc(-c4ccc5ccc6oc7ccccc7c6c5c4)cc3)c3ccc4sc5ccccc5c4c3)cc21 ; CC1(C)c2ccccc2-c2ccc(N(c3ccc(-c4ccc5ccc6oc7ccccc7c6c5c4)cc3)c3ccc4oc5ccccc5c4c3)cc21 ; CC1(C)c2ccccc2-c2c(N(c3ccc(-c4ccc5ccc6oc7ccccc7c6c5c4)cc3)c3ccc4sc5ccccc5c4c3)cccc21]

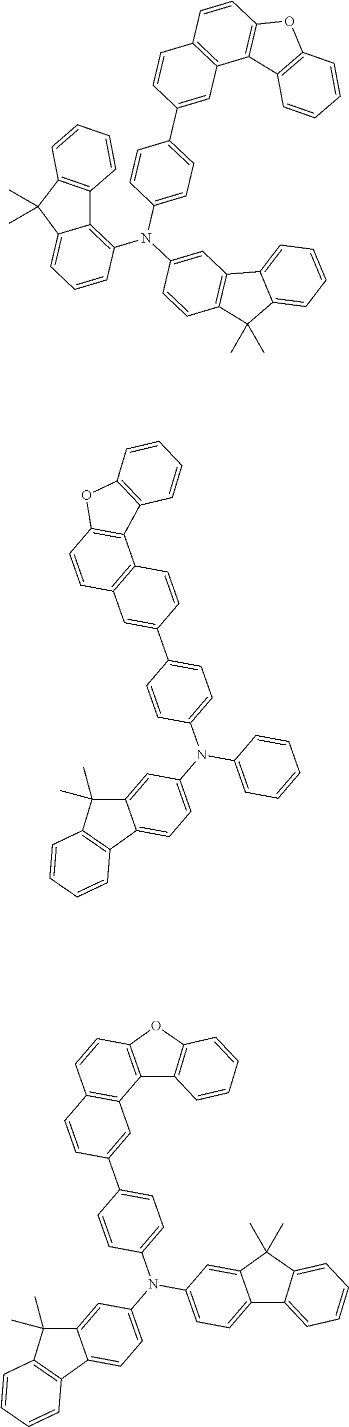 [SMILES: CC1(C)c2ccccc2-c2cc(N(c3ccc(-c4ccc5ccc6oc7ccccc7c6c5c4)cc3)c3cccc4c3-c3ccccc3C4(C)C)ccc21 ; CC1(C)c2ccccc2-c2ccc(N(c3ccccc3)c3ccc(-c4ccc5c(ccc6oc7ccccc7c65)c4)cc3)cc21 ; CC1(C)c2ccccc2-c2ccc(N(c3ccc(-c4ccc5ccc6oc7ccccc7c6c5c4)cc3)c3ccc4c(c3)C(C)(C)c3ccccc3-4)cc21]

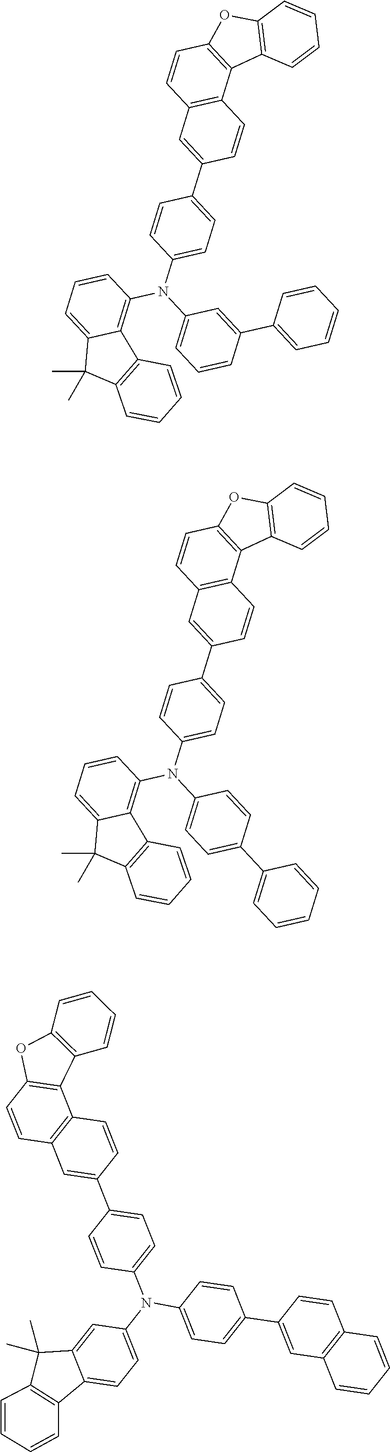 [SMILES: CC1(C)c2ccccc2-c2c(N(c3ccc(-c4ccc5c(ccc6oc7ccccc7c65)c4)cc3)c3cccc(-c4ccccc4)c3)cccc21 ; CC1(C)c2ccccc2-c2c(N(c3ccc(-c4ccccc4)cc3)c3ccc(-c4ccc5c(ccc6oc7ccccc7c65)c4)cc3)cccc21 ; CC1(C)c2ccccc2-c2ccc(N(c3ccc(-c4ccc5ccccc5c4)cc3)c3ccc(-c4ccc5c(ccc6oc7ccccc7c65)c4)cc3)cc21]

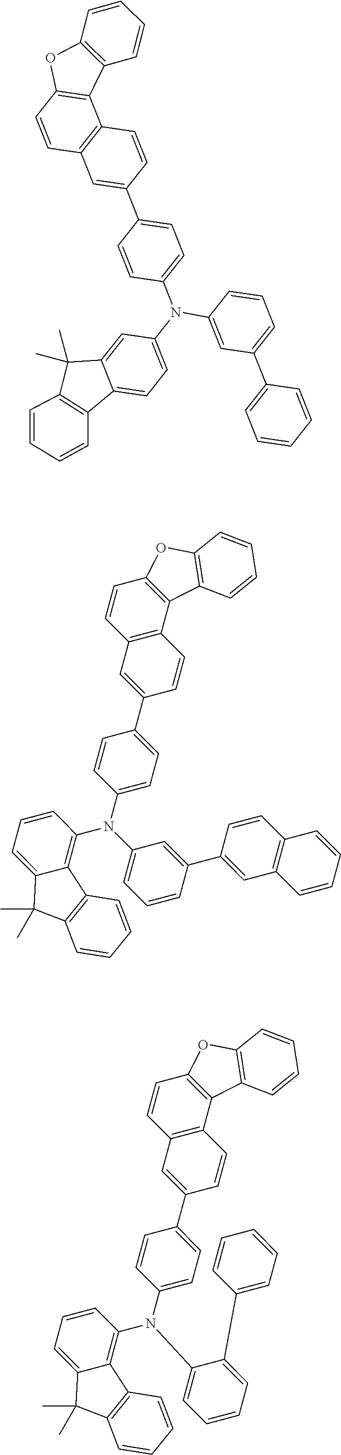 [SMILES: CC1(C)c2ccccc2-c2ccc(N(c3ccc(-c4ccc5c(ccc6oc7ccccc7c65)c4)cc3)c3cccc(-c4ccccc4)c3)cc21 ; CC1(C)c2ccccc2-c2c(N(c3ccc(-c4ccc5c(ccc6oc7ccccc7c65)c4)cc3)c3cccc(-c4ccc5ccccc5c4)c3)cccc21 ; CC1(C)c2ccccc2-c2c(N(c3ccc(-c4ccc5c(ccc6oc7ccccc7c65)c4)cc3)c3ccccc3-c3ccccc3)cccc21]

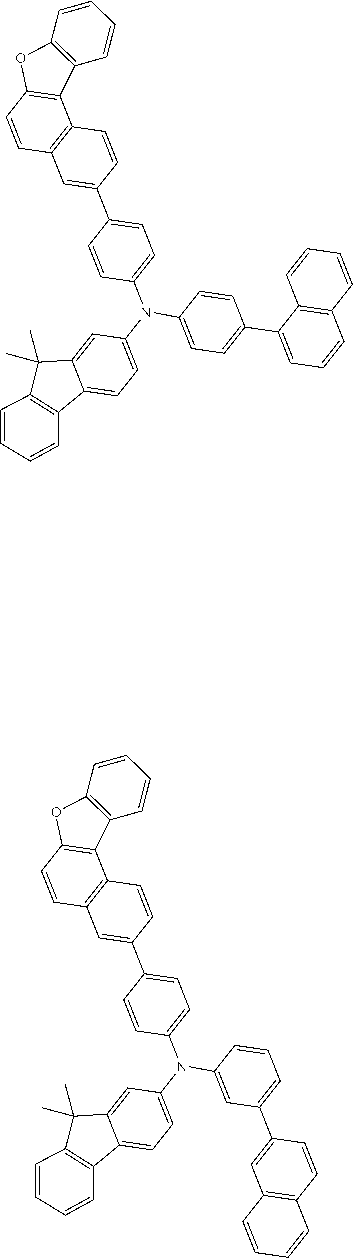 [SMILES: CC1(C)c2ccccc2-c2ccc(N(c3ccc(-c4ccc5c(ccc6oc7ccccc7c65)c4)cc3)c3ccc(-c4cccc5ccccc45)cc3)cc21 ; CC1(C)c2ccccc2-c2ccc(N(c3ccc(-c4ccc5c(ccc6oc7ccccc7c65)c4)cc3)c3cccc(-c4ccc5ccccc5c4)c3)cc21]

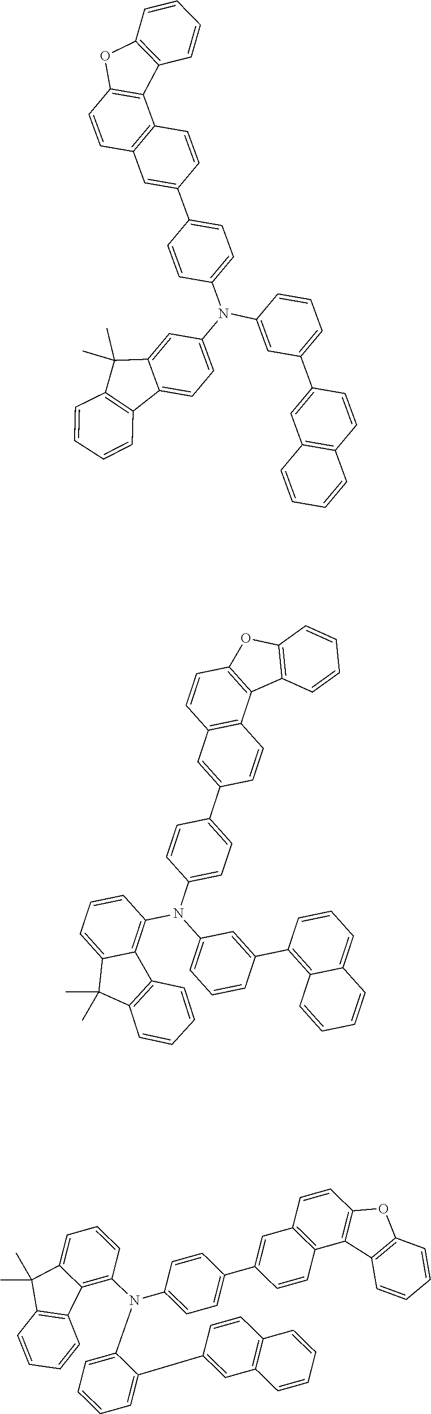 [SMILES: CC1(C)c2ccccc2-c2ccc(N(c3ccc(-c4ccc5c(ccc6oc7ccccc7c65)c4)cc3)c3cccc(-c4ccc5ccccc5c4)c3)cc21 ; CC1(C)c2ccccc2-c2c(N(c3ccc(-c4ccc5c(ccc6oc7ccccc7c65)c4)cc3)c3cccc(-c4cccc5ccccc45)c3)cccc21 ; CC1(C)c2ccccc2-c2c(N(c3ccc(-c4ccc5c(ccc6oc7ccccc7c65)c4)cc3)c3ccccc3-c3ccc4ccccc4c3)cccc21]

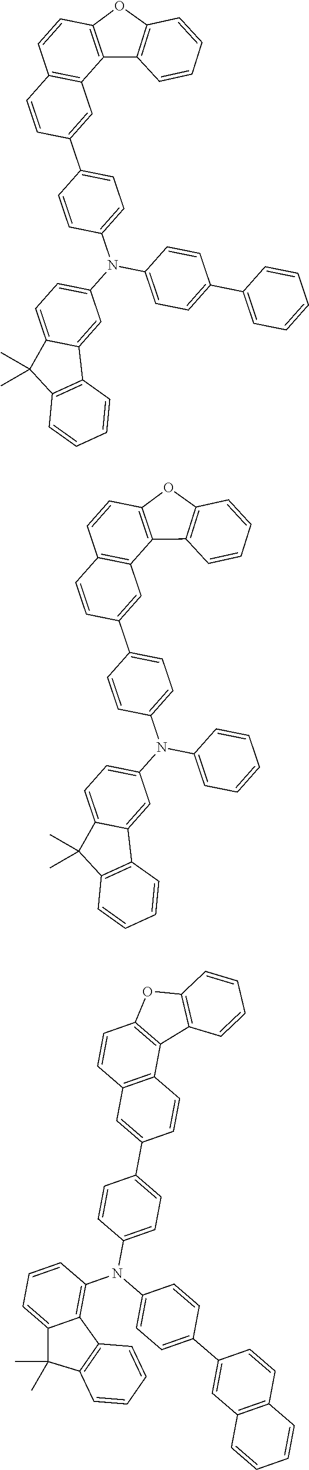 [SMILES: CC1(C)c2ccccc2-c2cc(N(c3ccc(-c4ccccc4)cc3)c3ccc(-c4ccc5ccc6oc7ccccc7c6c5c4)cc3)ccc21 ; CC1(C)c2ccccc2-c2cc(N(c3ccccc3)c3ccc(-c4ccc5ccc6oc7ccccc7c6c5c4)cc3)ccc21 ; CC1(C)c2ccccc2-c2c(N(c3ccc(-c4ccc5ccccc5c4)cc3)c3ccc(-c4ccc5c(ccc6oc7ccccc7c65)c4)cc3)cccc21]

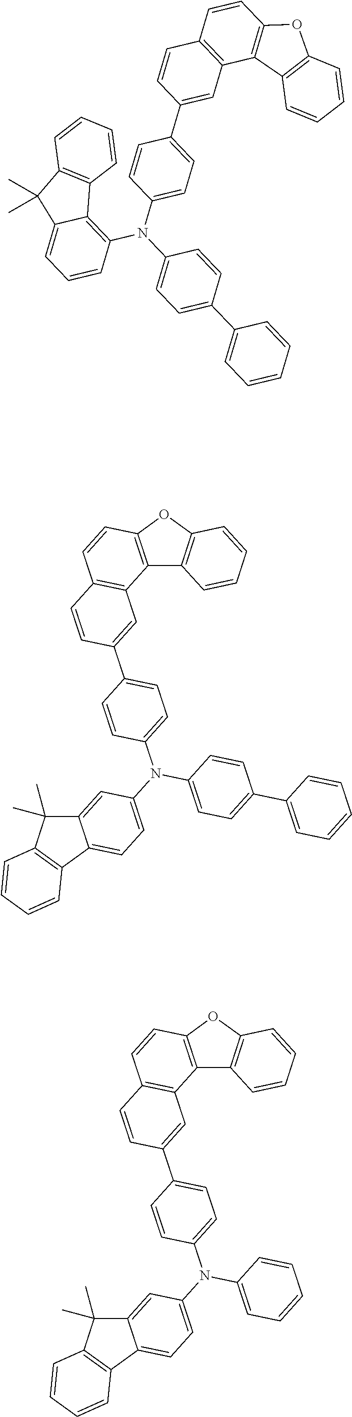 [SMILES: CC1(C)c2ccccc2-c2c(N(c3ccc(-c4ccccc4)cc3)c3ccc(-c4ccc5ccc6oc7ccccc7c6c5c4)cc3)cccc21 ; CC1(C)c2ccccc2-c2ccc(N(c3ccc(-c4ccccc4)cc3)c3ccc(-c4ccc5ccc6oc7ccccc7c6c5c4)cc3)cc21 ; CC1(C)c2ccccc2-c2ccc(N(c3ccccc3)c3ccc(-c4ccc5ccc6oc7ccccc7c6c5c4)cc3)cc21]

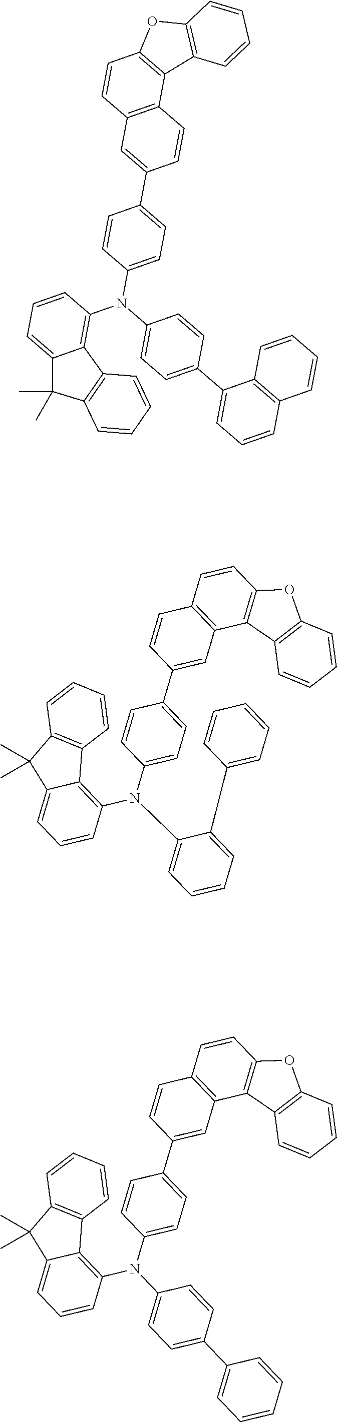 [SMILES: CC1(C)c2ccccc2-c2c(N(c3ccc(-c4ccc5c(ccc6oc7ccccc7c65)c4)cc3)c3ccc(-c4cccc5ccccc45)cc3)cccc21 ; CC1(C)c2ccccc2-c2c(N(c3ccc(-c4ccc5ccc6oc7ccccc7c6c5c4)cc3)c3ccccc3-c3ccccc3)cccc21 ; CC1(C)c2ccccc2-c2c(N(c3ccc(-c4ccccc4)cc3)c3ccc(-c4ccc5ccc6oc7ccccc7c6c5c4)cc3)cccc21]

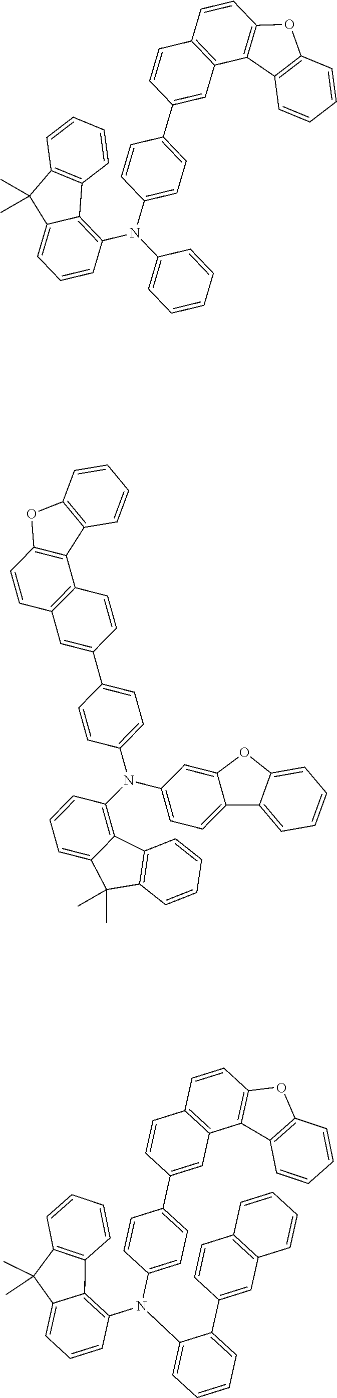 [SMILES: CC1(C)c2ccccc2-c2c(N(c3ccccc3)c3ccc(-c4ccc5ccc6oc7ccccc7c6c5c4)cc3)cccc21 ; CC1(C)c2ccccc2-c2c(N(c3ccc(-c4ccc5c(ccc6oc7ccccc7c65)c4)cc3)c3ccc4c(c3)oc3ccccc34)cccc21 ; CC1(C)c2ccccc2-c2c(N(c3ccc(-c4ccc5ccc6oc7ccccc7c6c5c4)cc3)c3ccccc3-c3ccc4ccccc4c3)cccc21]

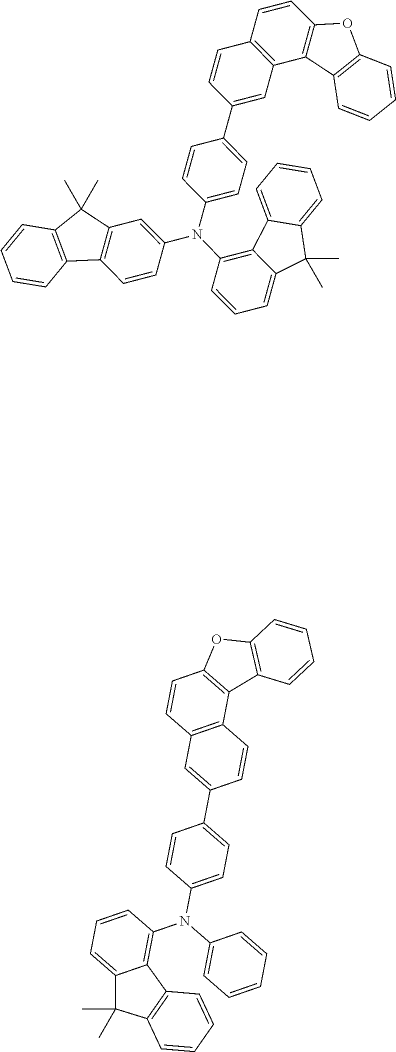 [SMILES: CC1(C)c2ccccc2-c2ccc(N(c3ccc(-c4ccc5ccc6oc7ccccc7c6c5c4)cc3)c3cccc4c3-c3ccccc3C4(C)C)cc21 ; CC1(C)c2ccccc2-c2c(N(c3ccccc3)c3ccc(-c4ccc5c(ccc6oc7ccccc7c65)c4)cc3)cccc21]

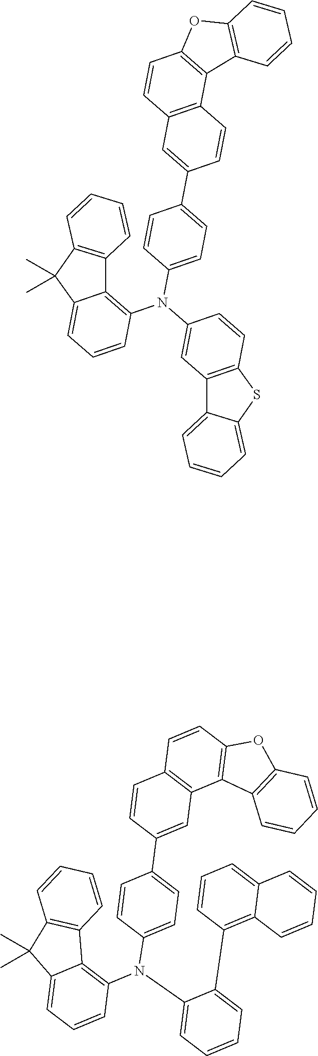 [SMILES: CC1(C)c2ccccc2-c2c(N(c3ccc(-c4ccc5c(ccc6oc7ccccc7c65)c4)cc3)c3ccc4sc5ccccc5c4c3)cccc21 ; CC1(C)c2ccccc2-c2c(N(c3ccc(-c4ccc5ccc6oc7ccccc7c6c5c4)cc3)c3ccccc3-c3cccc4ccccc34)cccc21]

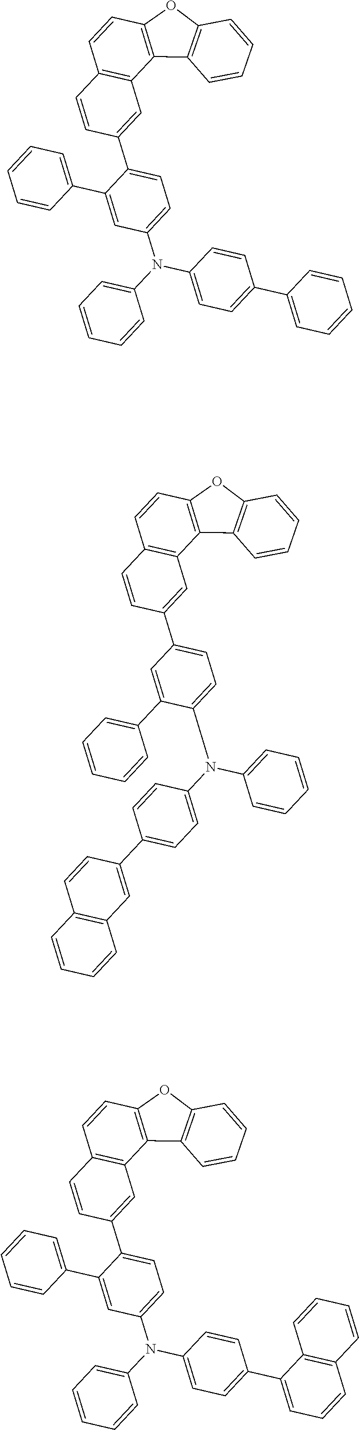 [SMILES: c1ccc(-c2ccc(N(c3ccccc3)c3ccc(-c4ccc5ccc6oc7ccccc7c6c5c4)c(-c4ccccc4)c3)cc2)cc1 ; c1ccc(-c2cc(-c3ccc4ccc5oc6ccccc6c5c4c3)ccc2N(c2ccccc2)c2ccc(-c3ccc4ccccc4c3)cc2)cc1 ; c1ccc(-c2cc(N(c3ccccc3)c3ccc(-c4cccc5ccccc45)cc3)ccc2-c2ccc3ccc4oc5ccccc5c4c3c2)cc1]

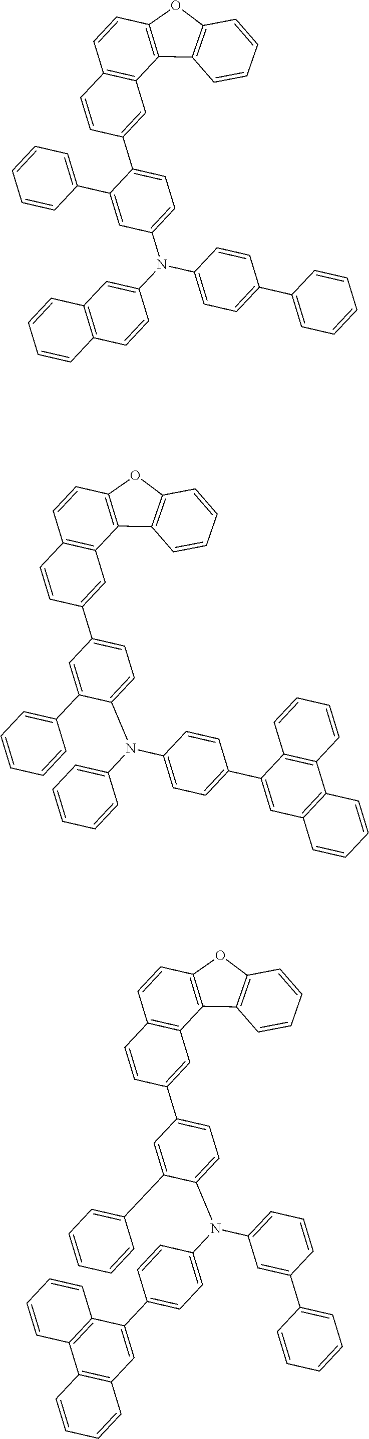 [SMILES: c1ccc(-c2ccc(N(c3ccc(-c4ccc5ccc6oc7ccccc7c6c5c4)c(-c4ccccc4)c3)c3ccc4ccccc4c3)cc2)cc1 ; c1ccc(-c2cc(-c3ccc4ccc5oc6ccccc6c5c4c3)ccc2N(c2ccccc2)c2ccc(-c3cc4ccccc4c4ccccc34)cc2)cc1 ; c1ccc(-c2cccc(N(c3ccc(-c4cc5ccccc5c5ccccc45)cc3)c3ccc(-c4ccc5ccc6oc7ccccc7c6c5c4)cc3-c3ccccc3)c2)cc1]

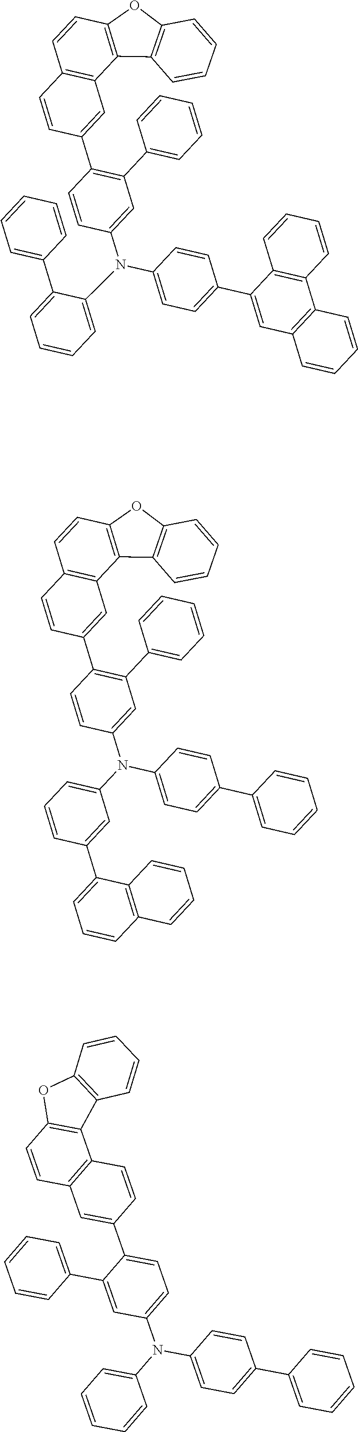 [SMILES: c1ccc(-c2cc(N(c3ccc(-c4cc5ccccc5c5ccccc45)cc3)c3ccccc3-c3ccccc3)ccc2-c2ccc3ccc4oc5ccccc5c4c3c2)cc1 ; c1ccc(-c2ccc(N(c3cccc(-c4cccc5ccccc45)c3)c3ccc(-c4ccc5ccc6oc7ccccc7c6c5c4)c(-c4ccccc4)c3)cc2)cc1 ; c1ccc(-c2ccc(N(c3ccccc3)c3ccc(-c4ccc5c(ccc6oc7ccccc7c65)c4)c(-c4ccccc4)c3)cc2)cc1]

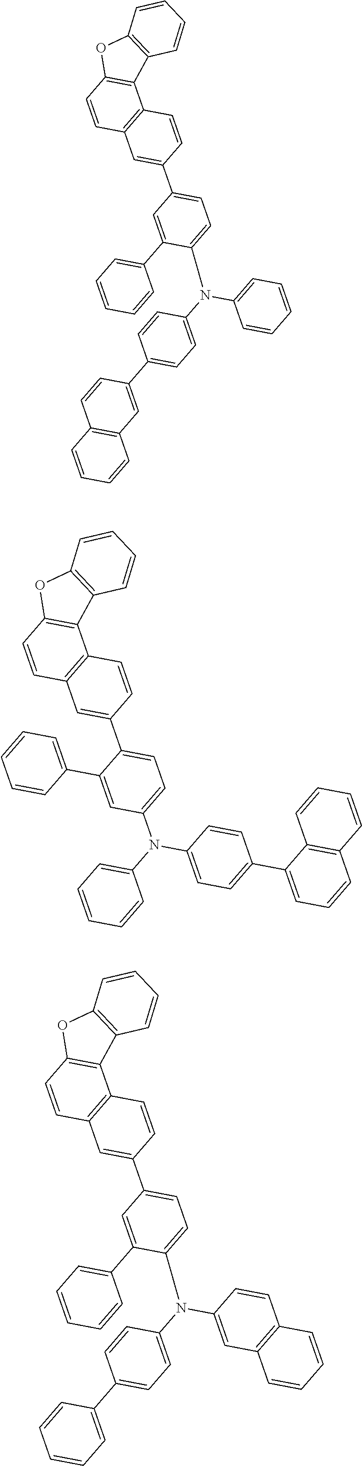

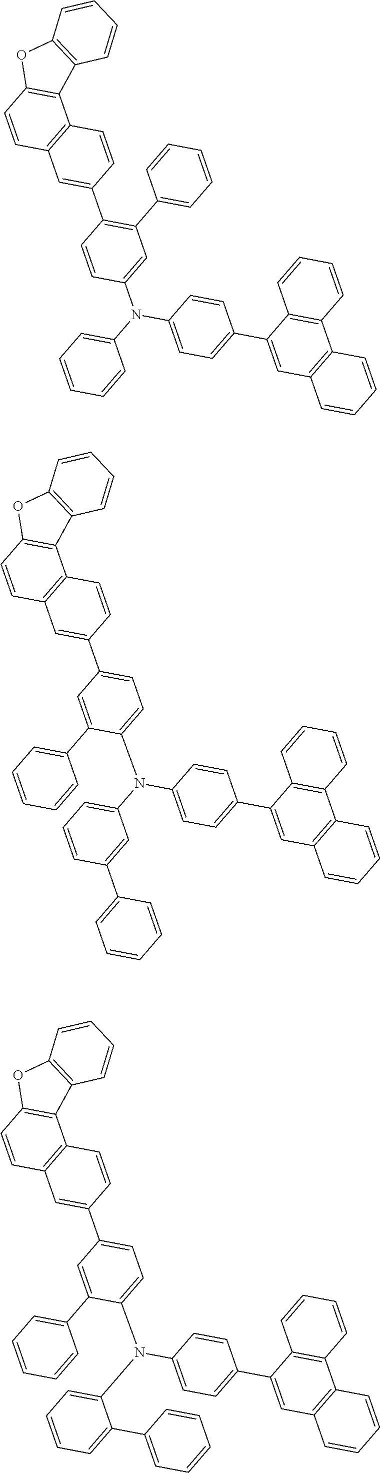 [SMILES: c1ccc(-c2cc(N(c3ccccc3)c3ccc(-c4cc5ccccc5c5ccccc45)cc3)ccc2-c2ccc3c(ccc4oc5ccccc5c43)c2)cc1 ; c1ccc(-c2cccc(N(c3ccc(-c4cc5ccccc5c5ccccc45)cc3)c3ccc(-c4ccc5c(ccc6oc7ccccc7c65)c4)cc3-c3ccccc3)c2)cc1 ; c1ccc(-c2ccccc2N(c2ccc(-c3cc4ccccc4c4ccccc34)cc2)c2ccc(-c3ccc4c(ccc5oc6ccccc6c54)c3)cc2-c2ccccc2)cc1]

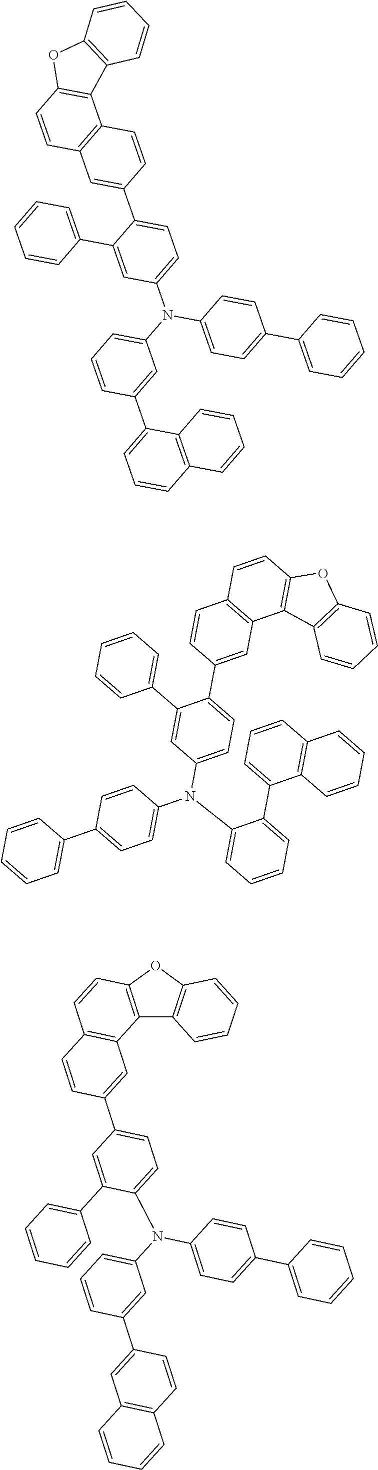 [SMILES: c1ccc(-c2ccc(N(c3cccc(-c4cccc5ccccc45)c3)c3ccc(-c4ccc5c(ccc6oc7ccccc7c65)c4)c(-c4ccccc4)c3)cc2)cc1 ; c1ccc(-c2ccc(N(c3ccc(-c4ccc5ccc6oc7ccccc7c6c5c4)c(-c4ccccc4)c3)c3ccccc3-c3cccc4ccccc34)cc2)cc1 ; c1ccc(-c2ccc(N(c3cccc(-c4ccc5ccccc5c4)c3)c3ccc(-c4ccc5ccc6oc7ccccc7c6c5c4)cc3-c3ccccc3)cc2)cc1]

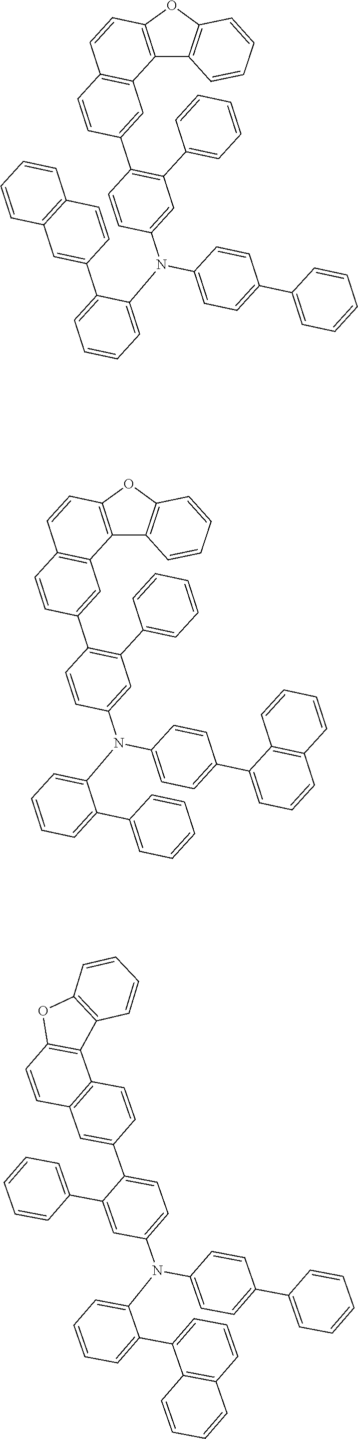 [SMILES: c1ccc(-c2ccc(N(c3ccc(-c4ccc5ccc6oc7ccccc7c6c5c4)c(-c4ccccc4)c3)c3ccccc3-c3ccc4ccccc4c3)cc2)cc1 ; c1ccc(-c2cc(N(c3ccc(-c4cccc5ccccc45)cc3)c3ccccc3-c3ccccc3)ccc2-c2ccc3ccc4oc5ccccc5c4c3c2)cc1 ; c1ccc(-c2ccc(N(c3ccc(-c4ccc5c(ccc6oc7ccccc7c65)c4)c(-c4ccccc4)c3)c3ccccc3-c3cccc4ccccc34)cc2)cc1]

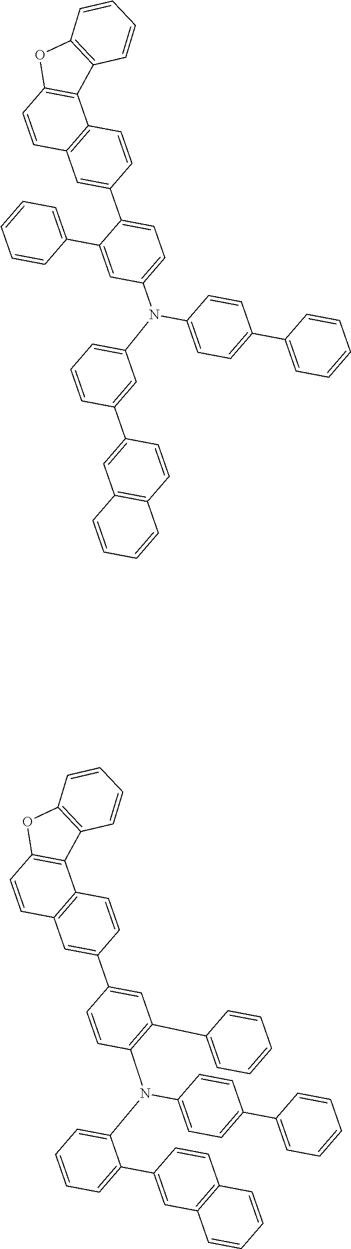 [SMILES: c1ccc(-c2ccc(N(c3cccc(-c4ccc5ccccc5c4)c3)c3ccc(-c4ccc5c(ccc6oc7ccccc7c65)c4)c(-c4ccccc4)c3)cc2)cc1 ; c1ccc(-c2ccc(N(c3ccccc3-c3ccc4ccccc4c3)c3ccc(-c4ccc5c(ccc6oc7ccccc7c65)c4)cc3-c3ccccc3)cc2)cc1]

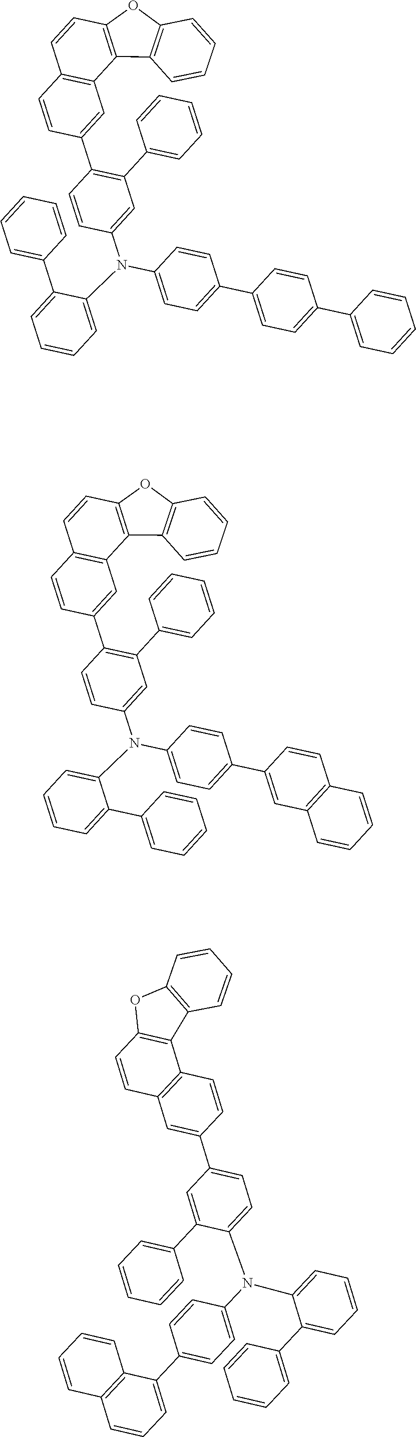 [SMILES: c1ccc(-c2ccc(-c3ccc(N(c4ccc(-c5ccc6ccc7oc8ccccc8c7c6c5)c(-c5ccccc5)c4)c4ccccc4-c4ccccc4)cc3)cc2)cc1 ; c1ccc(-c2cc(N(c3ccc(-c4ccc5ccccc5c4)cc3)c3ccccc3-c3ccccc3)ccc2-c2ccc3ccc4oc5ccccc5c4c3c2)cc1 ; c1ccc(-c2ccccc2N(c2ccc(-c3cccc4ccccc34)cc2)c2ccc(-c3ccc4c(ccc5oc6ccccc6c54)c3)cc2-c2ccccc2)cc1]

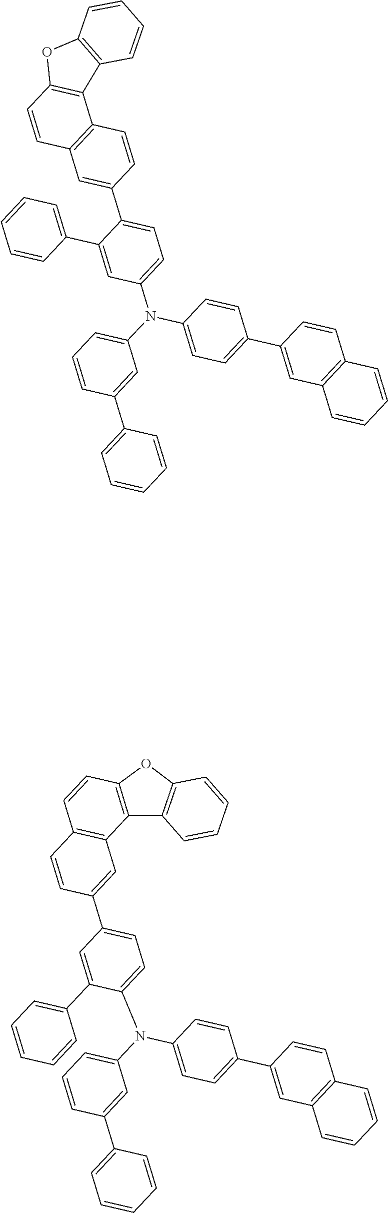 [SMILES: c1ccc(-c2cccc(N(c3ccc(-c4ccc5ccccc5c4)cc3)c3ccc(-c4ccc5c(ccc6oc7ccccc7c65)c4)c(-c4ccccc4)c3)c2)cc1 ; c1ccc(-c2cccc(N(c3ccc(-c4ccc5ccccc5c4)cc3)c3ccc(-c4ccc5ccc6oc7ccccc7c6c5c4)cc3-c3ccccc3)c2)cc1]

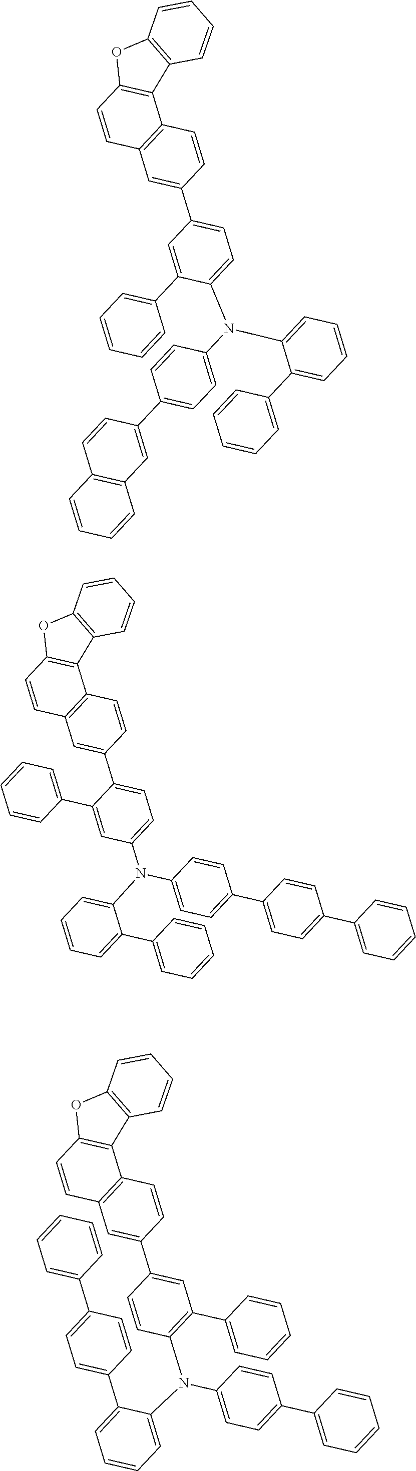

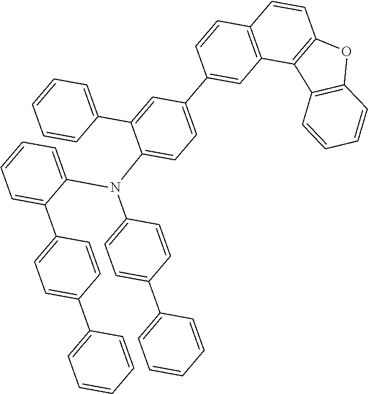 [SMILES: c1ccc(-c2ccc(-c3ccccc3N(c3ccc(-c4ccccc4)cc3)c3ccc(-c4ccc5ccc6oc7ccccc7c6c5c4)cc3-c3ccccc3)cc2)cc1]

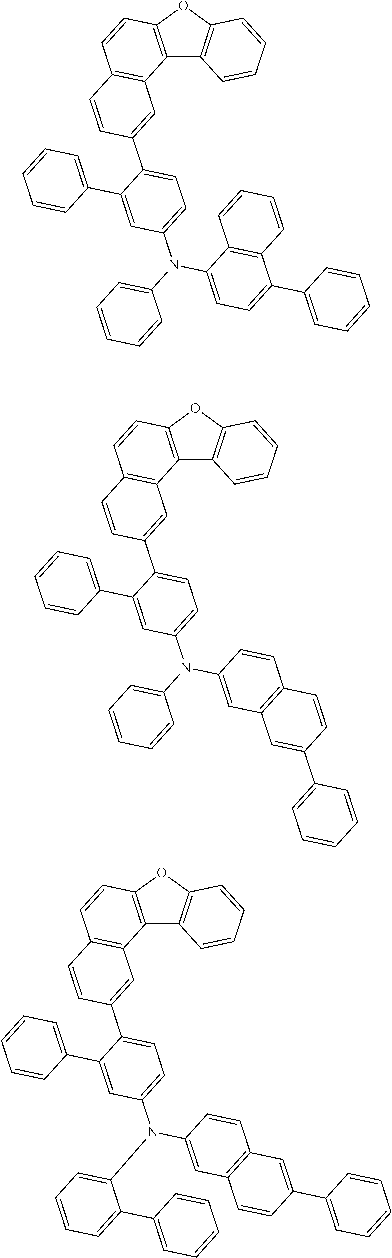 [SMILES: c1ccc(-c2cc(N(c3ccccc3)c3ccc(-c4ccccc4)c4ccccc34)ccc2-c2ccc3ccc4oc5ccccc5c4c3c2)cc1 ; c1ccc(-c2ccc3ccc(N(c4ccccc4)c4ccc(-c5ccc6ccc7oc8ccccc8c7c6c5)c(-c5ccccc5)c4)cc3c2)cc1 ; c1ccc(-c2ccc3cc(N(c4ccc(-c5ccc6ccc7oc8ccccc8c7c6c5)c(-c5ccccc5)c4)c4ccccc4-c4ccccc4)ccc3c2)cc1]

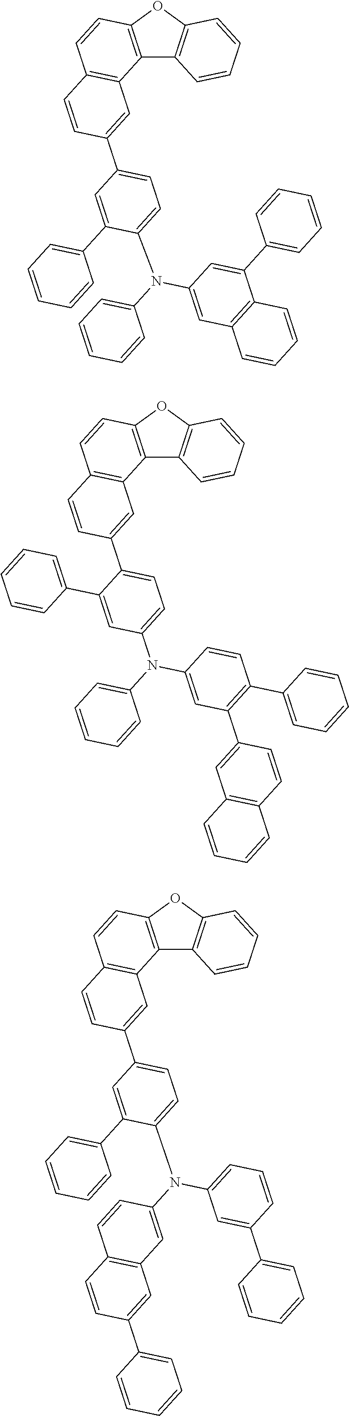 [SMILES: c1ccc(-c2cc(-c3ccc4ccc5oc6ccccc6c5c4c3)ccc2N(c2ccccc2)c2cc(-c3ccccc3)c3ccccc3c2)cc1 ; c1ccc(-c2ccc(N(c3ccccc3)c3ccc(-c4ccc5ccc6oc7ccccc7c6c5c4)c(-c4ccccc4)c3)cc2-c2ccc3ccccc3c2)cc1 ; c1ccc(-c2cccc(N(c3ccc4ccc(-c5ccccc5)cc4c3)c3ccc(-c4ccc5ccc6oc7ccccc7c6c5c4)cc3-c3ccccc3)c2)cc1]

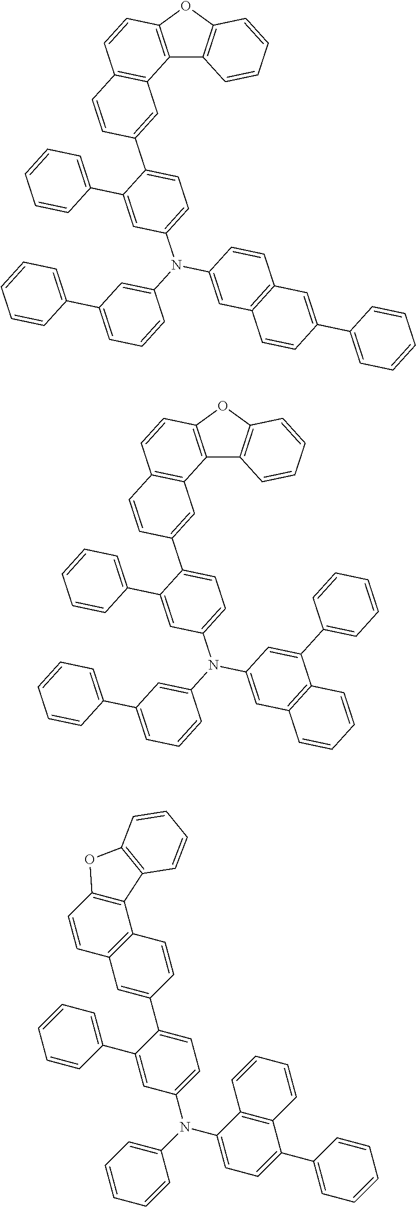 [SMILES: c1ccc(-c2cccc(N(c3ccc(-c4ccc5ccc6oc7ccccc7c6c5c4)c(-c4ccccc4)c3)c3ccc4cc(-c5ccccc5)ccc4c3)c2)cc1 ; c1ccc(-c2cccc(N(c3ccc(-c4ccc5ccc6oc7ccccc7c6c5c4)c(-c4ccccc4)c3)c3cc(-c4ccccc4)c4ccccc4c3)c2)cc1 ; c1ccc(-c2cc(N(c3ccccc3)c3ccc(-c4ccccc4)c4ccccc34)ccc2-c2ccc3c(ccc4oc5ccccc5c43)c2)cc1]

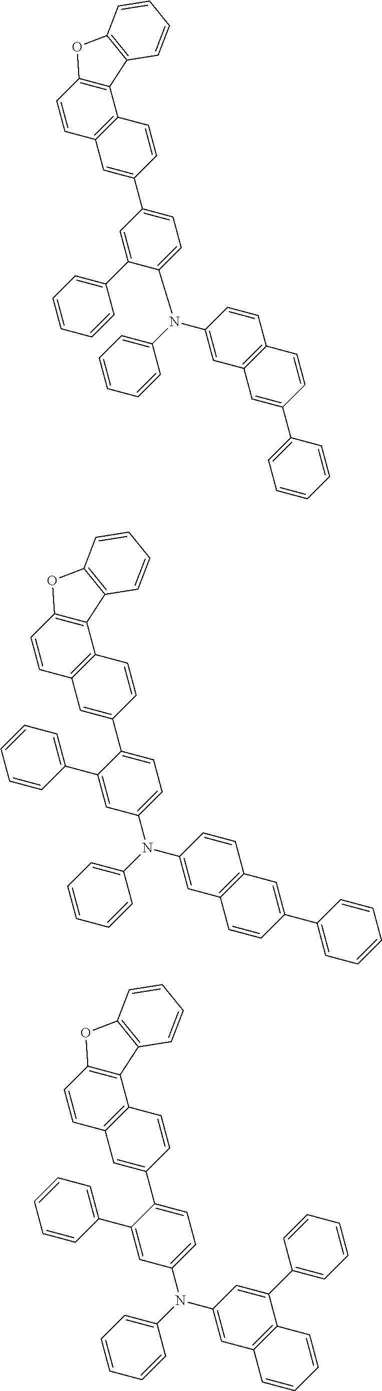 [SMILES: c1ccc(-c2ccc3ccc(N(c4ccccc4)c4ccc(-c5ccc6c(ccc7oc8ccccc8c76)c5)cc4-c4ccccc4)cc3c2)cc1 ; c1ccc(-c2ccc3cc(N(c4ccccc4)c4ccc(-c5ccc6c(ccc7oc8ccccc8c76)c5)c(-c5ccccc5)c4)ccc3c2)cc1 ; c1ccc(-c2cc(N(c3ccccc3)c3cc(-c4ccccc4)c4ccccc4c3)ccc2-c2ccc3c(ccc4oc5ccccc5c43)c2)cc1]

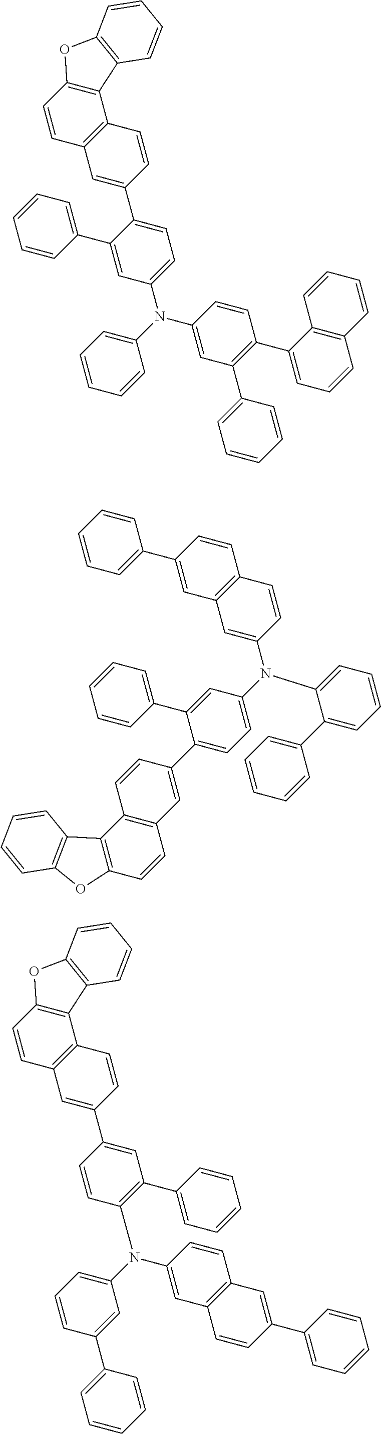

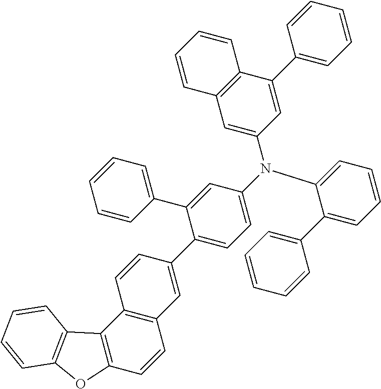 [SMILES: c1ccc(-c2cc(N(c3cc(-c4ccccc4)c4ccccc4c3)c3ccccc3-c3ccccc3)ccc2-c2ccc3c(ccc4oc5ccccc5c43)c2)cc1]

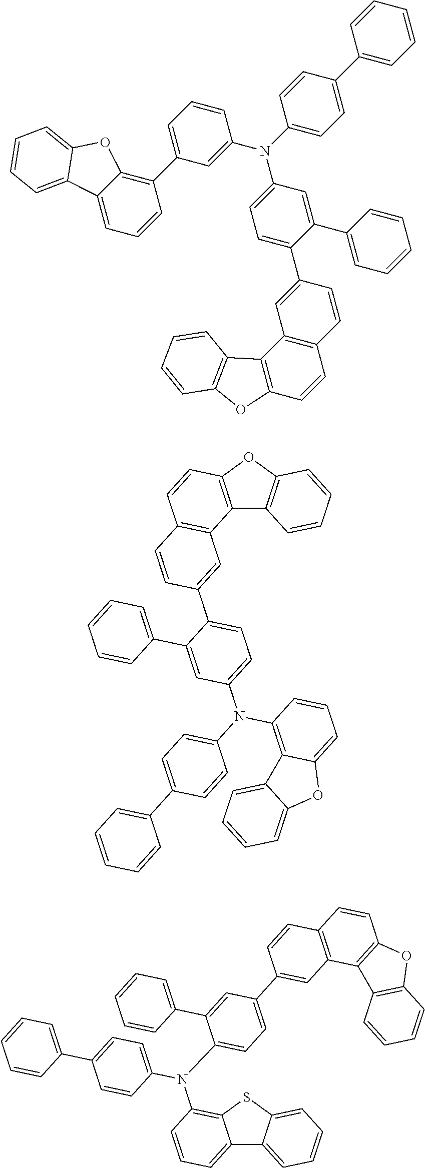 [SMILES: c1ccc(-c2ccc(N(c3cccc(-c4cccc5c4oc4ccccc45)c3)c3ccc(-c4ccc5ccc6oc7ccccc7c6c5c4)c(-c4ccccc4)c3)cc2)cc1 ; c1ccc(-c2ccc(N(c3ccc(-c4ccc5ccc6oc7ccccc7c6c5c4)c(-c4ccccc4)c3)c3cccc4oc5ccccc5c34)cc2)cc1 ; c1ccc(-c2ccc(N(c3ccc(-c4ccc5ccc6oc7ccccc7c6c5c4)cc3-c3ccccc3)c3cccc4c3sc3ccccc34)cc2)cc1]

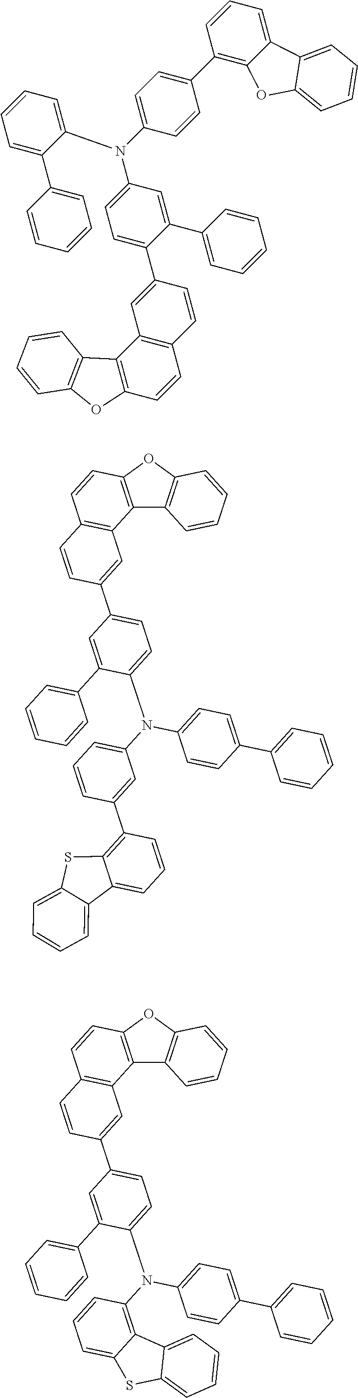 [SMILES: c1ccc(-c2cc(N(c3ccc(-c4cccc5c4oc4ccccc45)cc3)c3ccccc3-c3ccccc3)ccc2-c2ccc3ccc4oc5ccccc5c4c3c2)cc1 ; c1ccc(-c2ccc(N(c3cccc(-c4cccc5c4sc4ccccc45)c3)c3ccc(-c4ccc5ccc6oc7ccccc7c6c5c4)cc3-c3ccccc3)cc2)cc1 ; c1ccc(-c2ccc(N(c3ccc(-c4ccc5ccc6oc7ccccc7c6c5c4)cc3-c3ccccc3)c3cccc4sc5ccccc5c34)cc2)cc1]

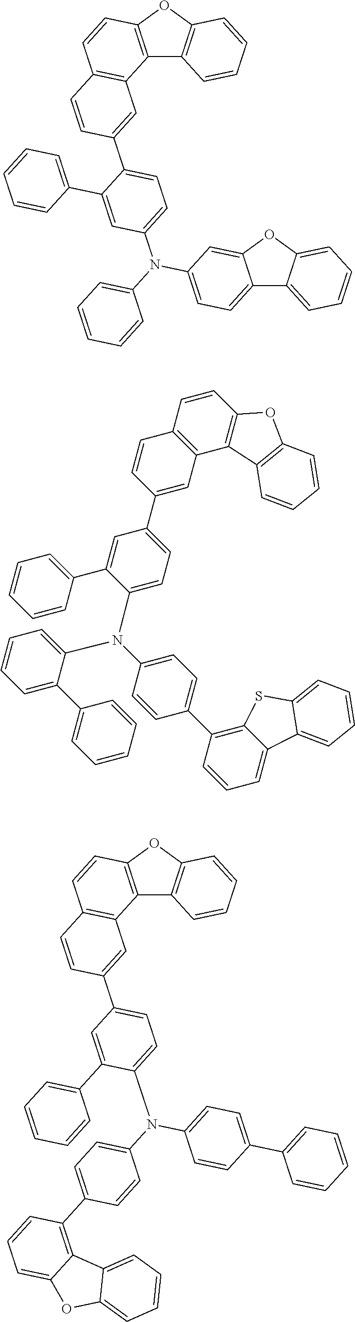 [SMILES: c1ccc(-c2cc(N(c3ccccc3)c3ccc4c(c3)oc3ccccc34)ccc2-c2ccc3ccc4oc5ccccc5c4c3c2)cc1 ; c1ccc(-c2ccccc2N(c2ccc(-c3cccc4c3sc3ccccc34)cc2)c2ccc(-c3ccc4ccc5oc6ccccc6c5c4c3)cc2-c2ccccc2)cc1 ; c1ccc(-c2ccc(N(c3ccc(-c4cccc5oc6ccccc6c45)cc3)c3ccc(-c4ccc5ccc6oc7ccccc7c6c5c4)cc3-c3ccccc3)cc2)cc1]

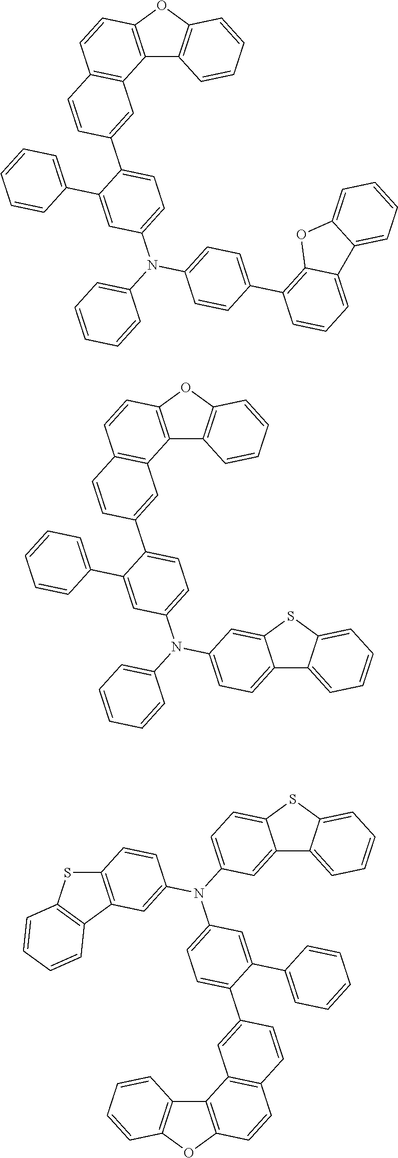 [SMILES: c1ccc(-c2cc(N(c3ccccc3)c3ccc(-c4cccc5c4oc4ccccc45)cc3)ccc2-c2ccc3ccc4oc5ccccc5c4c3c2)cc1 ; c1ccc(-c2cc(N(c3ccccc3)c3ccc4c(c3)sc3ccccc34)ccc2-c2ccc3ccc4oc5ccccc5c4c3c2)cc1 ; c1ccc(-c2cc(N(c3ccc4sc5ccccc5c4c3)c3ccc4sc5ccccc5c4c3)ccc2-c2ccc3ccc4oc5ccccc5c4c3c2)cc1]

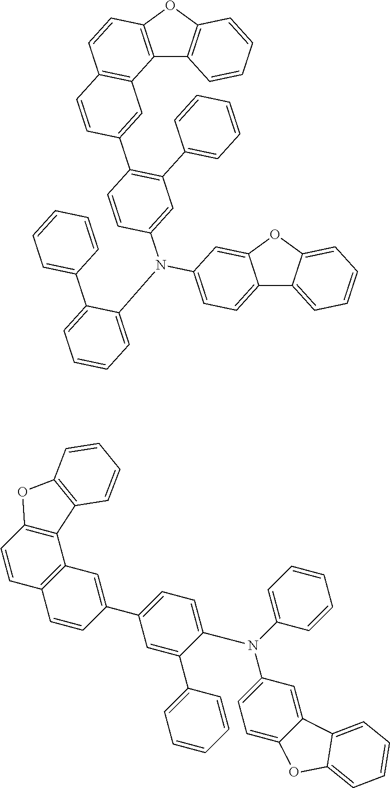 [SMILES: c1ccc(-c2cc(N(c3ccc4c(c3)oc3ccccc34)c3ccccc3-c3ccccc3)ccc2-c2ccc3ccc4oc5ccccc5c4c3c2)cc1 ; c1ccc(-c2cc(-c3ccc4ccc5oc6ccccc6c5c4c3)ccc2N(c2ccccc2)c2ccc3oc4ccccc4c3c2)cc1]

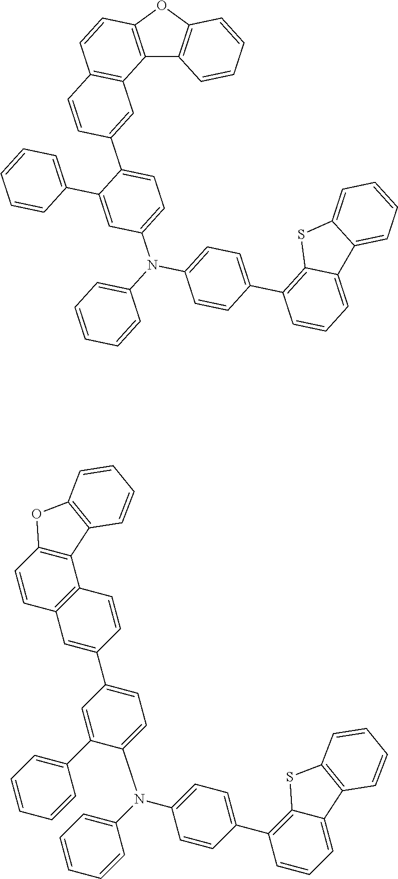 [SMILES: c1ccc(-c2cc(N(c3ccccc3)c3ccc(-c4cccc5c4sc4ccccc45)cc3)ccc2-c2ccc3ccc4oc5ccccc5c4c3c2)cc1 ; c1ccc(-c2cc(-c3ccc4c(ccc5oc6ccccc6c54)c3)ccc2N(c2ccccc2)c2ccc(-c3cccc4c3sc3ccccc34)cc2)cc1]

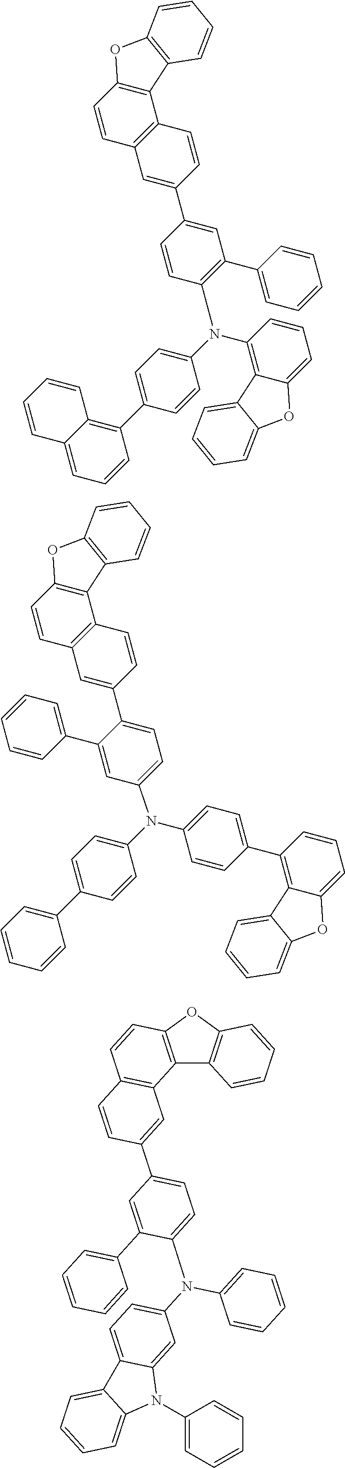

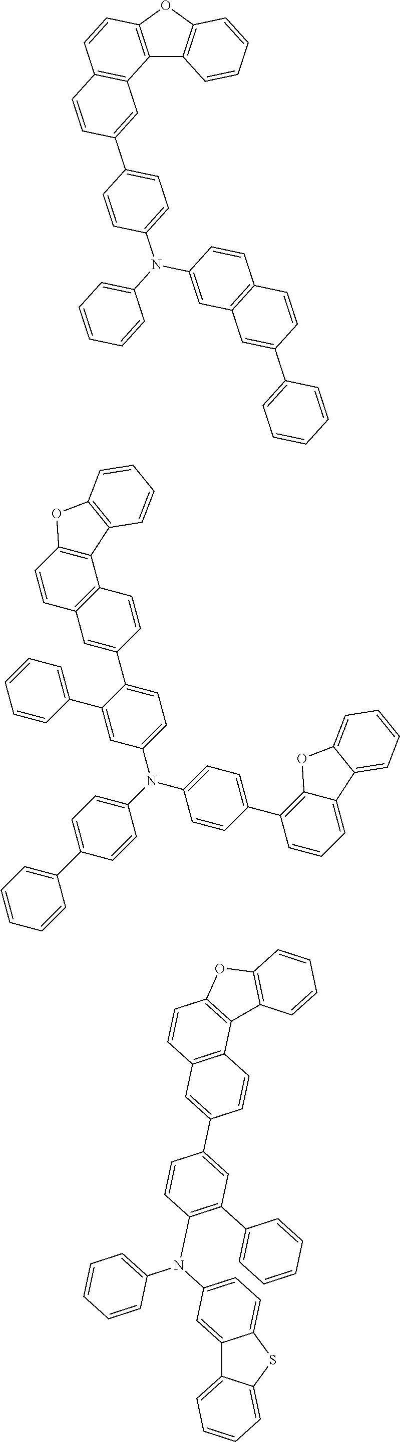 [SMILES: c1ccc(-c2ccc3ccc(N(c4ccccc4)c4ccc(-c5ccc6ccc7oc8ccccc8c7c6c5)cc4)cc3c2)cc1 ; c1ccc(-c2ccc(N(c3ccc(-c4cccc5c4oc4ccccc45)cc3)c3ccc(-c4ccc5c(ccc6oc7ccccc7c65)c4)c(-c4ccccc4)c3)cc2)cc1 ; c1ccc(-c2cc(-c3ccc4c(ccc5oc6ccccc6c54)c3)ccc2N(c2ccccc2)c2ccc3sc4ccccc4c3c2)cc1]

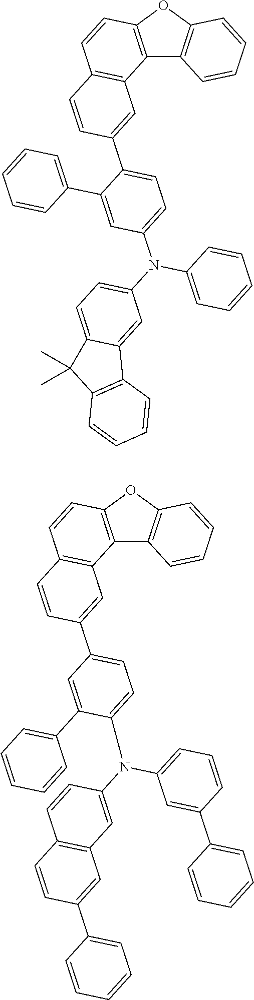 [SMILES: CC1(C)c2ccccc2-c2cc(N(c3ccccc3)c3ccc(-c4ccc5ccc6oc7ccccc7c6c5c4)c(-c4ccccc4)c3)ccc21 ; c1ccc(-c2cccc(N(c3ccc4ccc(-c5ccccc5)cc4c3)c3ccc(-c4ccc5ccc6oc7ccccc7c6c5c4)cc3-c3ccccc3)c2)cc1]

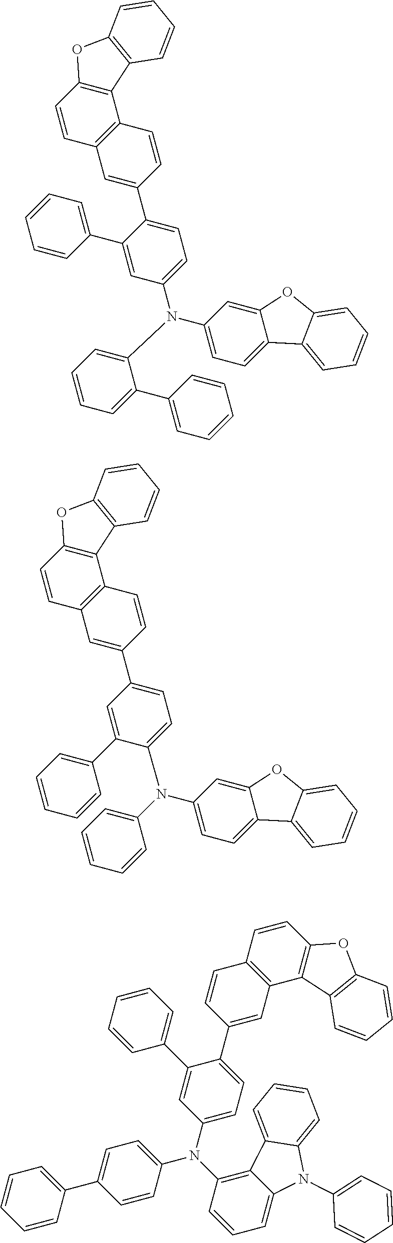 [SMILES: c1ccc(-c2cc(N(c3ccc4c(c3)oc3ccccc34)c3ccccc3-c3ccccc3)ccc2-c2ccc3c(ccc4oc5ccccc5c43)c2)cc1 ; c1ccc(-c2cc(-c3ccc4c(ccc5oc6ccccc6c54)c3)ccc2N(c2ccccc2)c2ccc3c(c2)oc2ccccc23)cc1 ; c1ccc(-c2ccc(N(c3ccc(-c4ccc5ccc6oc7ccccc7c6c5c4)c(-c4ccccc4)c3)c3cccc4c3c3ccccc3n4-c3ccccc3)cc2)cc1]

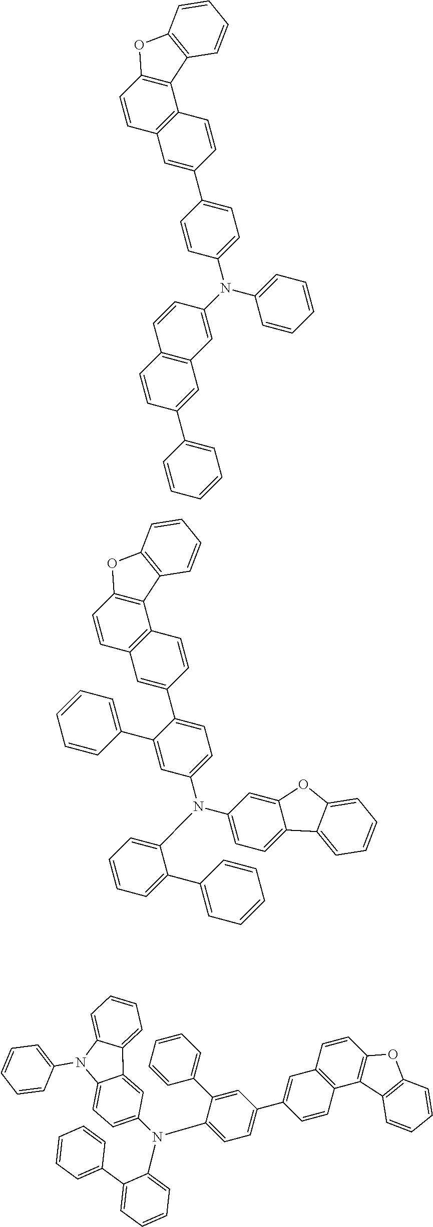

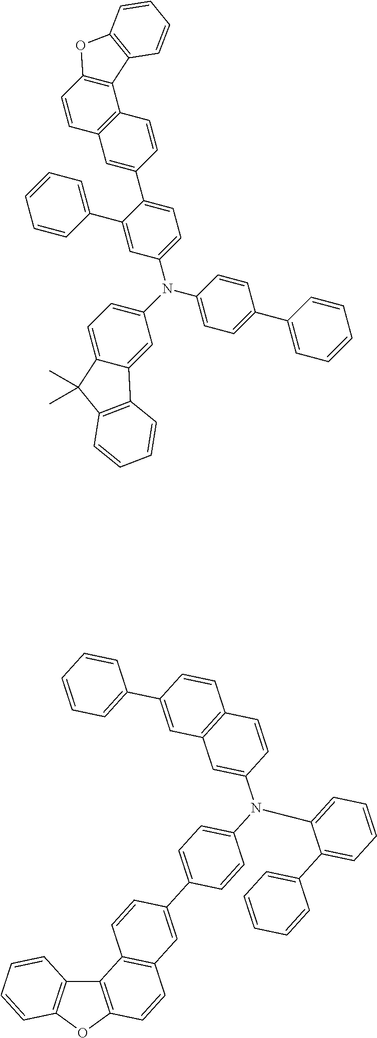 [SMILES: CC1(C)c2ccccc2-c2cc(N(c3ccc(-c4ccccc4)cc3)c3ccc(-c4ccc5c(ccc6oc7ccccc7c65)c4)c(-c4ccccc4)c3)ccc21 ; c1ccc(-c2ccc3ccc(N(c4ccc(-c5ccc6c(ccc7oc8ccccc8c76)c5)cc4)c4ccccc4-c4ccccc4)cc3c2)cc1]

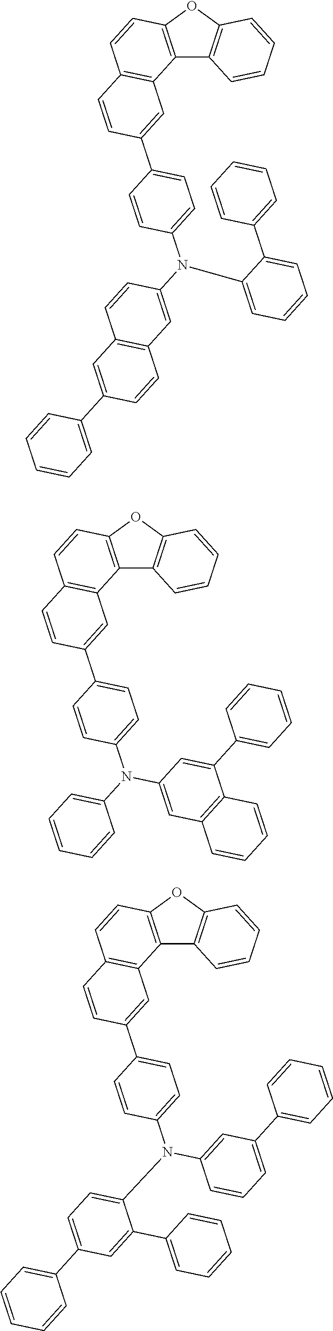

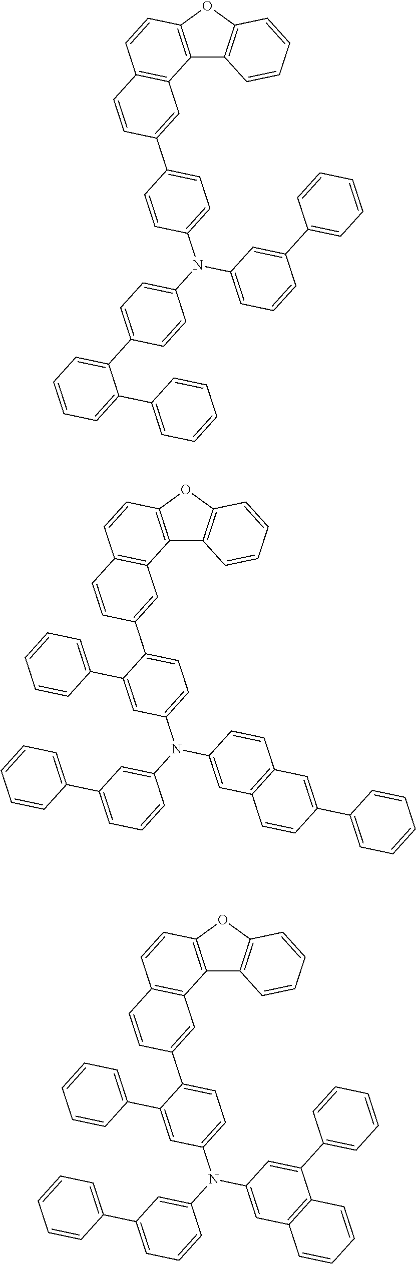 [SMILES: c1ccc(-c2cccc(N(c3ccc(-c4ccc5ccc6oc7ccccc7c6c5c4)cc3)c3ccc(-c4ccccc4-c4ccccc4)cc3)c2)cc1 ; c1ccc(-c2cccc(N(c3ccc(-c4ccc5ccc6oc7ccccc7c6c5c4)c(-c4ccccc4)c3)c3ccc4cc(-c5ccccc5)ccc4c3)c2)cc1 ; c1ccc(-c2cccc(N(c3ccc(-c4ccc5ccc6oc7ccccc7c6c5c4)c(-c4ccccc4)c3)c3cc(-c4ccccc4)c4ccccc4c3)c2)cc1]

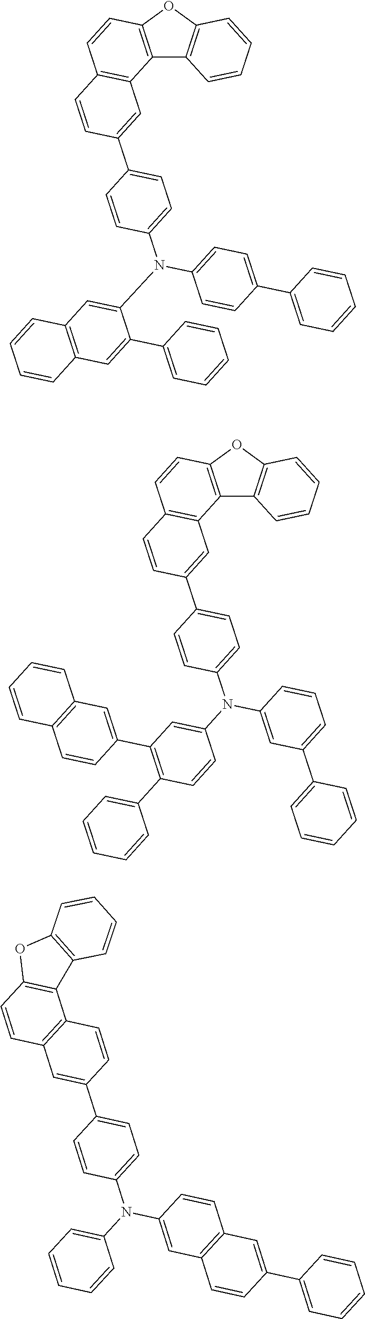

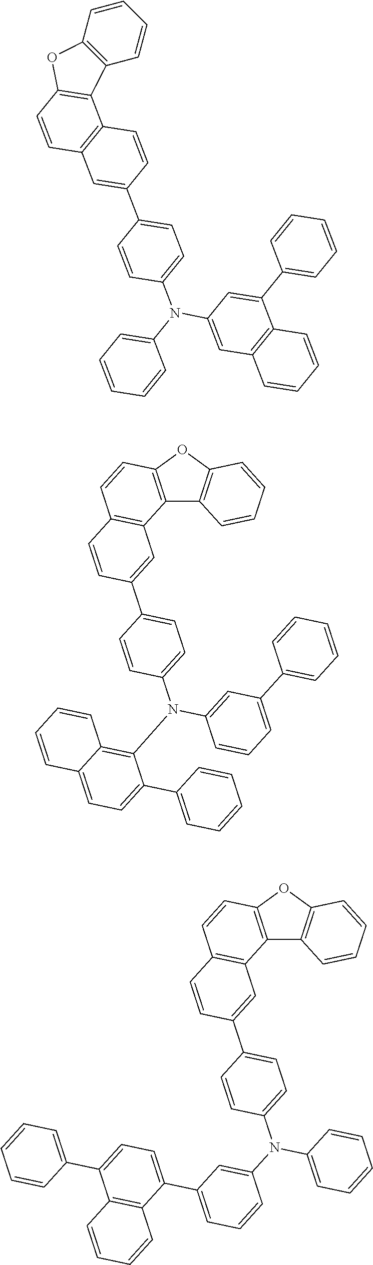 [SMILES: c1ccc(-c2cc(N(c3ccccc3)c3ccc(-c4ccc5c(ccc6oc7ccccc7c65)c4)cc3)cc3ccccc23)cc1 ; c1ccc(-c2cccc(N(c3ccc(-c4ccc5ccc6oc7ccccc7c6c5c4)cc3)c3c(-c4ccccc4)ccc4ccccc34)c2)cc1 ; c1ccc(-c2ccc(-c3cccc(N(c4ccccc4)c4ccc(-c5ccc6ccc7oc8ccccc8c7c6c5)cc4)c3)c3ccccc23)cc1]

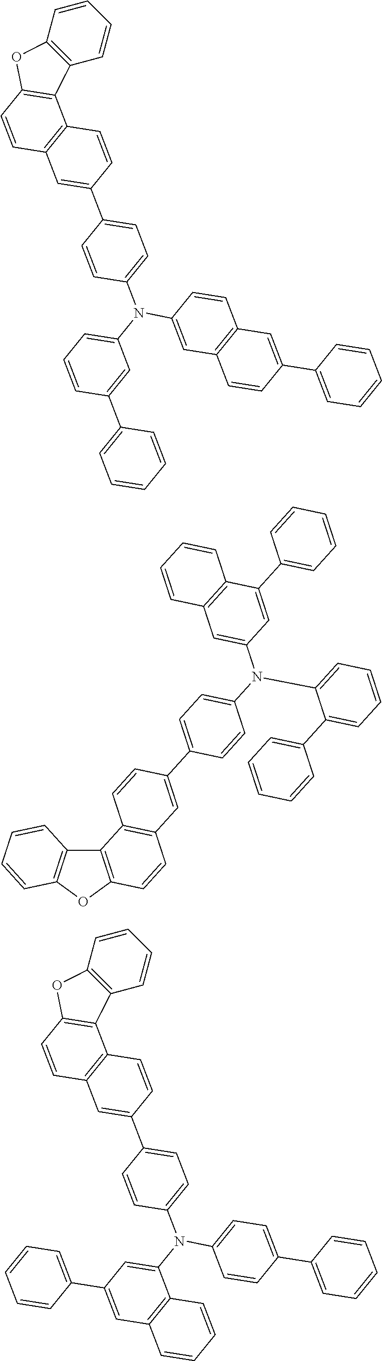

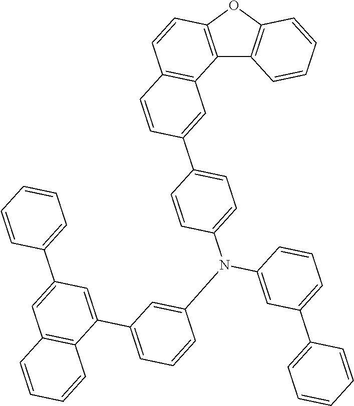 [SMILES: c1ccc(-c2cccc(N(c3ccc(-c4ccc5ccc6oc7ccccc7c6c5c4)cc3)c3cccc(-c4cc(-c5ccccc5)cc5ccccc45)c3)c2)cc1]

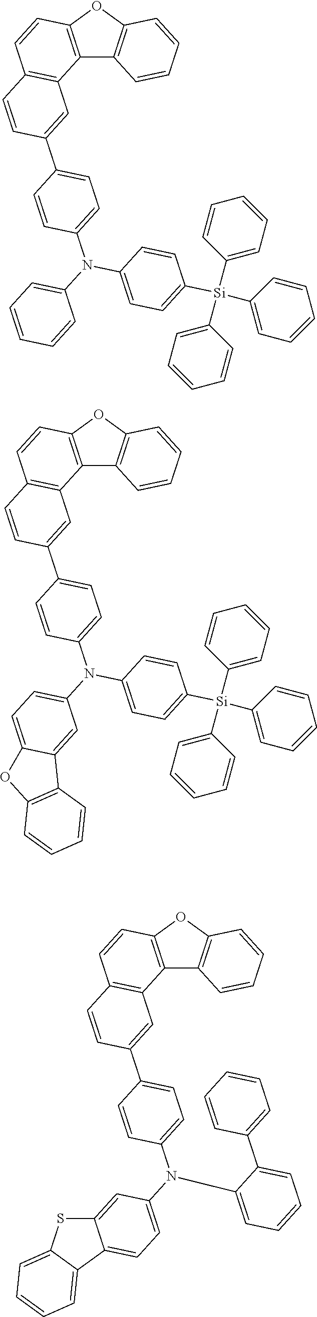 [SMILES: c1ccc(N(c2ccc(-c3ccc4ccc5oc6ccccc6c5c4c3)cc2)c2ccc([Si](c3ccccc3)(c3ccccc3)c3ccccc3)cc2)cc1 ; c1ccc([Si](c2ccccc2)(c2ccccc2)c2ccc(N(c3ccc(-c4ccc5ccc6oc7ccccc7c6c5c4)cc3)c3ccc4oc5ccccc5c4c3)cc2)cc1 ; c1ccc(-c2ccccc2N(c2ccc(-c3ccc4ccc5oc6ccccc6c5c4c3)cc2)c2ccc3c(c2)sc2ccccc23)cc1]

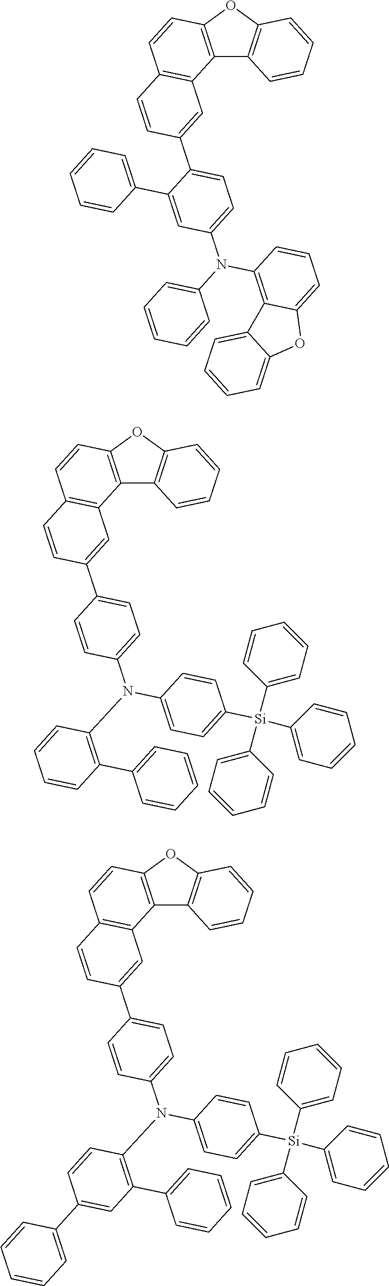

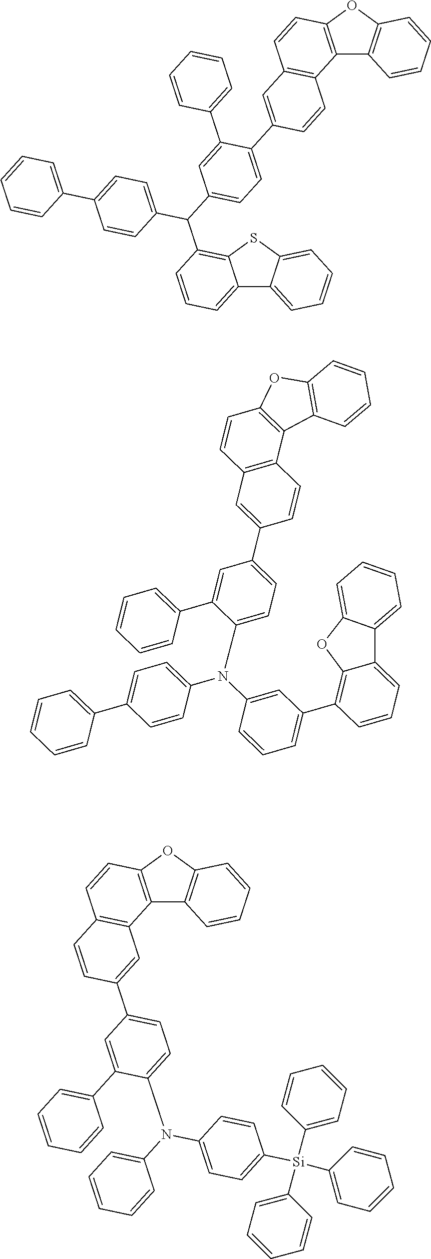 [SMILES: c1ccc(-c2ccc(C(c3ccc(-c4ccc5c(ccc6oc7ccccc7c65)c4)c(-c4ccccc4)c3)c3cccc4c3sc3ccccc34)cc2)cc1 ; c1ccc(-c2ccc(N(c3cccc(-c4cccc5c4oc4ccccc45)c3)c3ccc(-c4ccc5c(ccc6oc7ccccc7c65)c4)cc3-c3ccccc3)cc2)cc1 ; c1ccc(-c2cc(-c3ccc4ccc5oc6ccccc6c5c4c3)ccc2N(c2ccccc2)c2ccc([Si](c3ccccc3)(c3ccccc3)c3ccccc3)cc2)cc1]

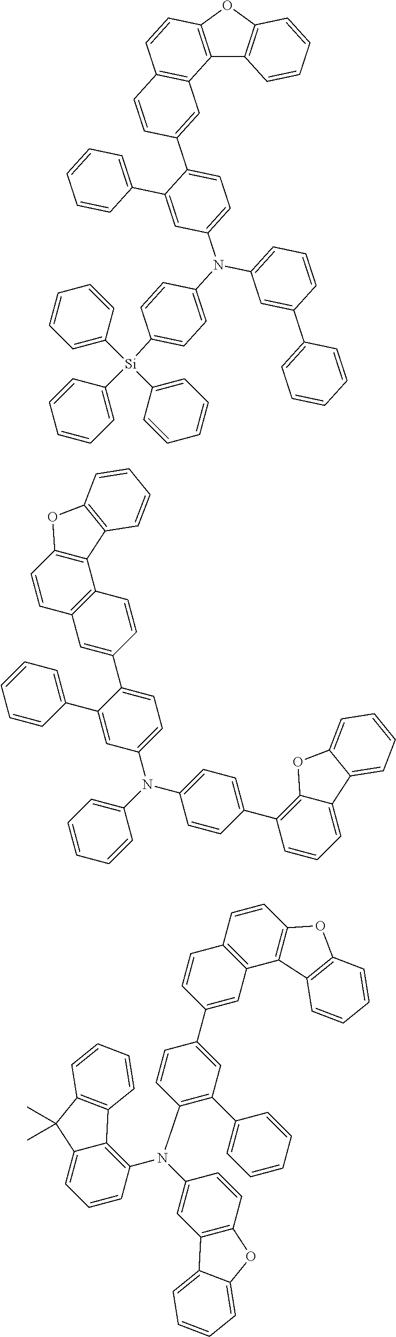

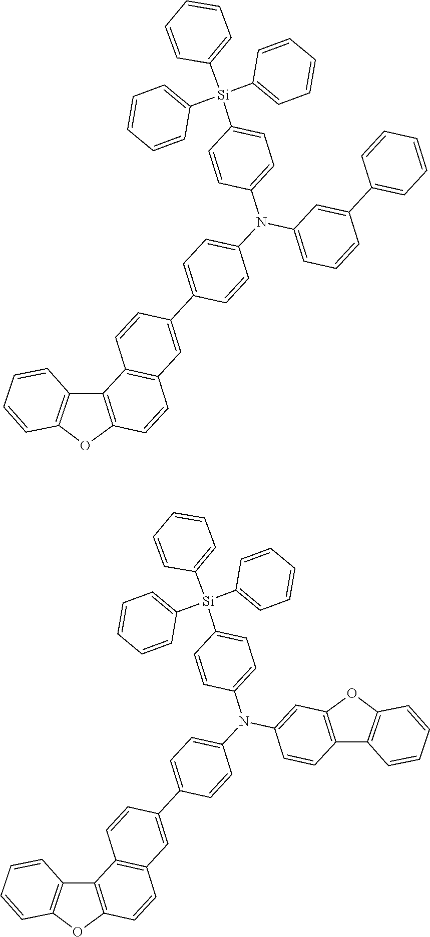 [SMILES: c1ccc(-c2cccc(N(c3ccc(-c4ccc5c(ccc6oc7ccccc7c65)c4)cc3)c3ccc([Si](c4ccccc4)(c4ccccc4)c4ccccc4)cc3)c2)cc1 ; c1ccc([Si](c2ccccc2)(c2ccccc2)c2ccc(N(c3ccc(-c4ccc5c(ccc6oc7ccccc7c65)c4)cc3)c3ccc4c(c3)oc3ccccc34)cc2)cc1]

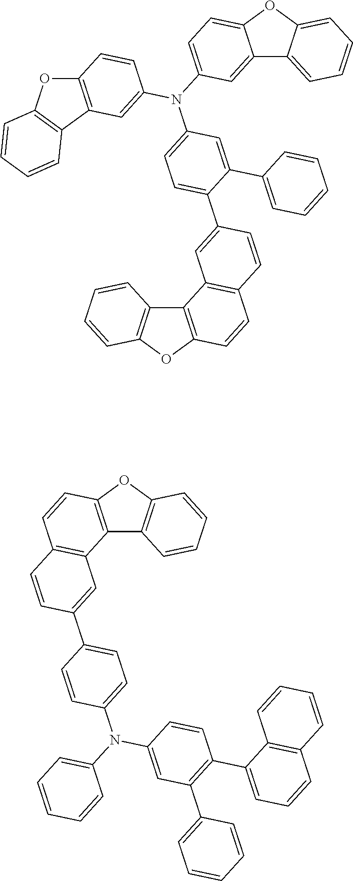 [SMILES: c1ccc(-c2cc(N(c3ccc4oc5ccccc5c4c3)c3ccc4oc5ccccc5c4c3)ccc2-c2ccc3ccc4oc5ccccc5c4c3c2)cc1 ; c1ccc(-c2cc(N(c3ccccc3)c3ccc(-c4ccc5ccc6oc7ccccc7c6c5c4)cc3)ccc2-c2cccc3ccccc23)cc1]

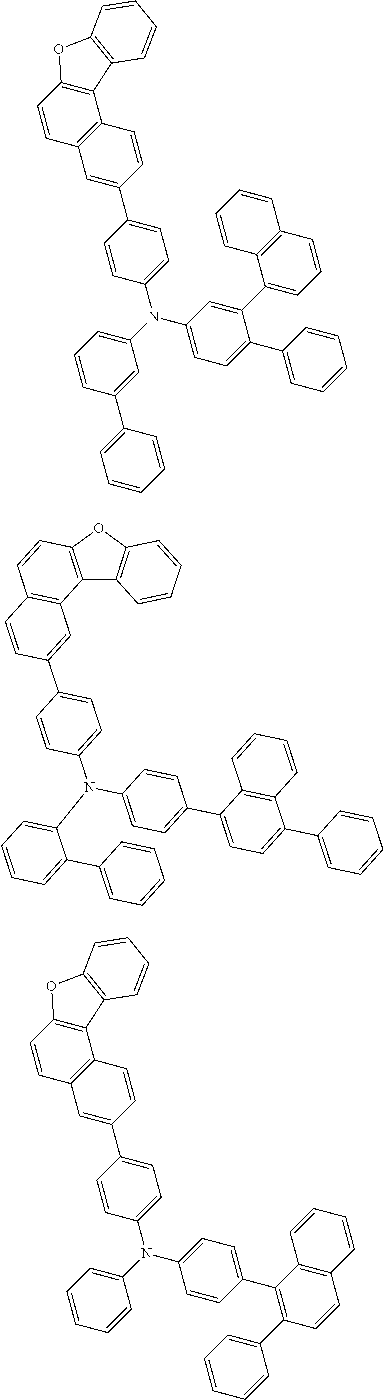

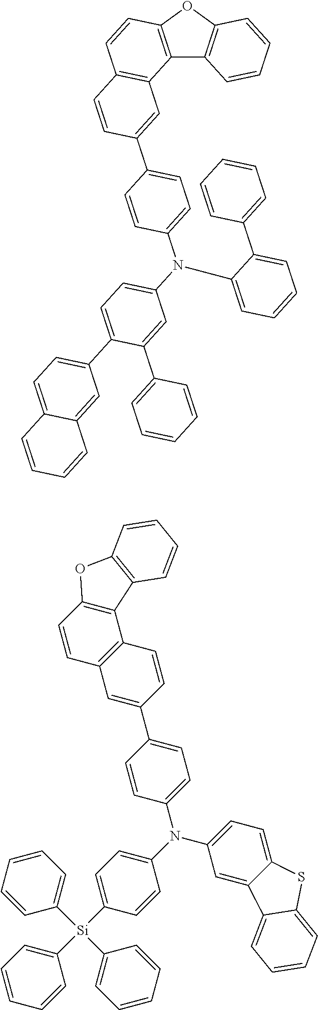 [SMILES: c1ccc(-c2cc(N(c3ccc(-c4ccc5ccc6oc7ccccc7c6c5c4)cc3)c3ccccc3-c3ccccc3)ccc2-c2ccc3ccccc3c2)cc1 ; c1ccc([Si](c2ccccc2)(c2ccccc2)c2ccc(N(c3ccc(-c4ccc5c(ccc6oc7ccccc7c65)c4)cc3)c3ccc4sc5ccccc5c4c3)cc2)cc1]

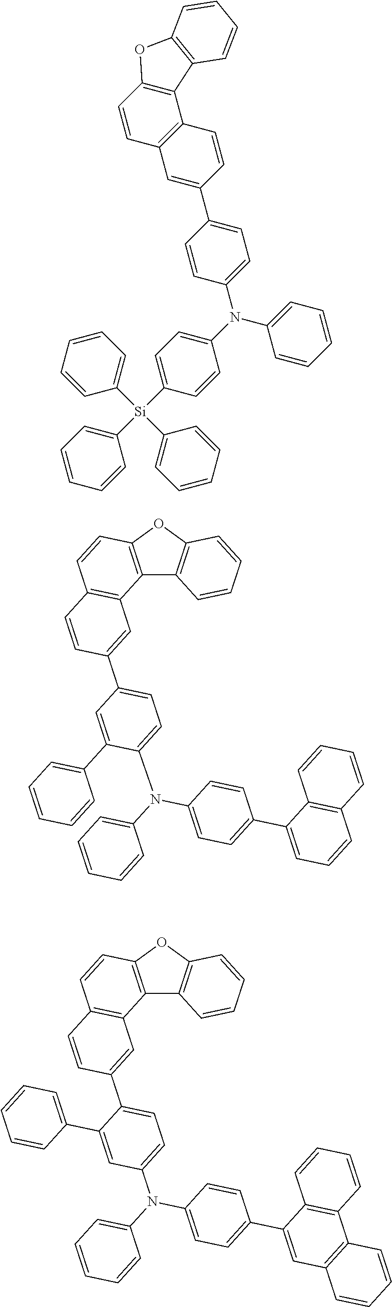

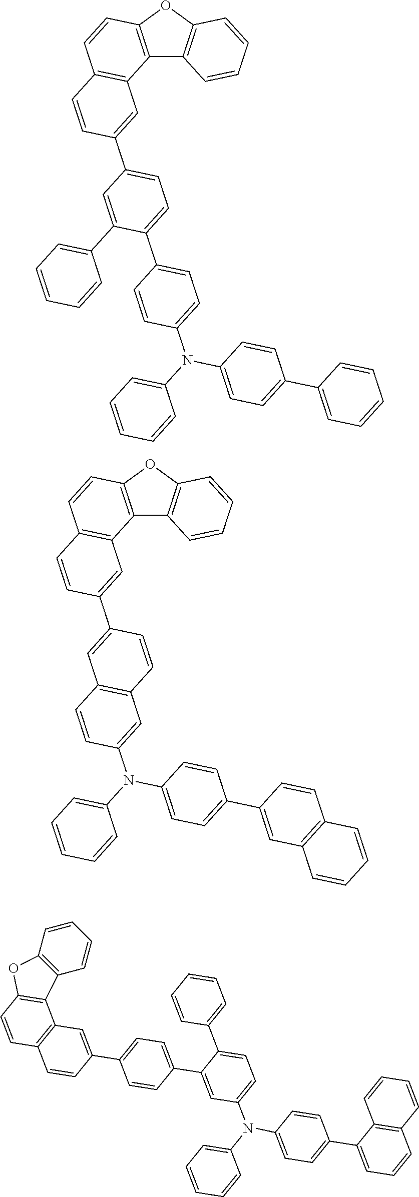

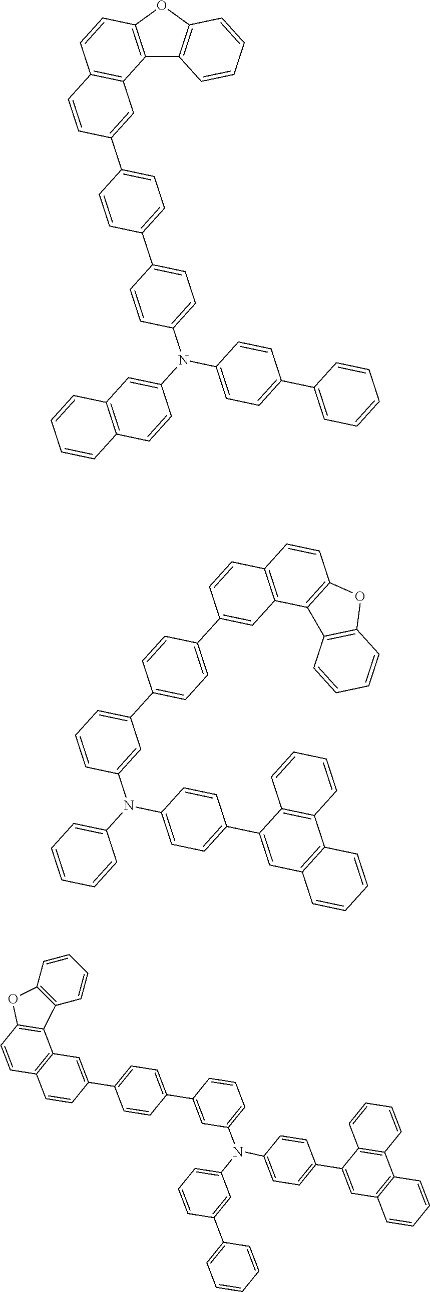 [SMILES: c1ccc(-c2ccc(N(c3ccc(-c4ccc(-c5ccc6ccc7oc8ccccc8c7c6c5)cc4)cc3)c3ccc4ccccc4c3)cc2)cc1 ; c1ccc(N(c2ccc(-c3cc4ccccc4c4ccccc34)cc2)c2cccc(-c3ccc(-c4ccc5ccc6oc7ccccc7c6c5c4)cc3)c2)cc1 ; c1ccc(-c2cccc(N(c3ccc(-c4cc5ccccc5c5ccccc45)cc3)c3cccc(-c4ccc(-c5ccc6ccc7oc8ccccc8c7c6c5)cc4)c3)c2)cc1]

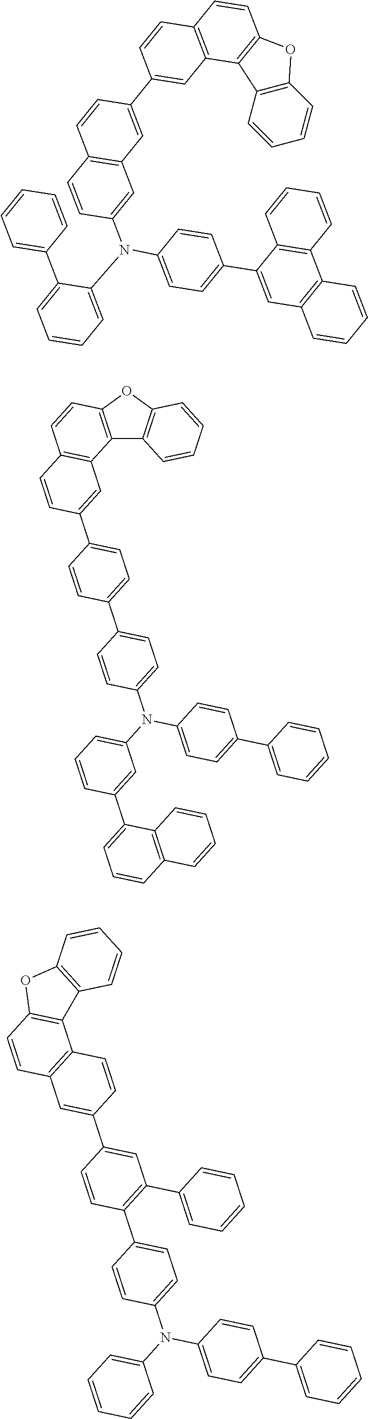 [SMILES: c1ccc(-c2ccccc2N(c2ccc(-c3cc4ccccc4c4ccccc34)cc2)c2ccc3ccc(-c4ccc5ccc6oc7ccccc7c6c5c4)cc3c2)cc1 ; c1ccc(-c2ccc(N(c3ccc(-c4ccc(-c5ccc6ccc7oc8ccccc8c7c6c5)cc4)cc3)c3cccc(-c4cccc5ccccc45)c3)cc2)cc1 ; c1ccc(-c2ccc(N(c3ccccc3)c3ccc(-c4ccc(-c5ccc6c(ccc7oc8ccccc8c76)c5)cc4-c4ccccc4)cc3)cc2)cc1]

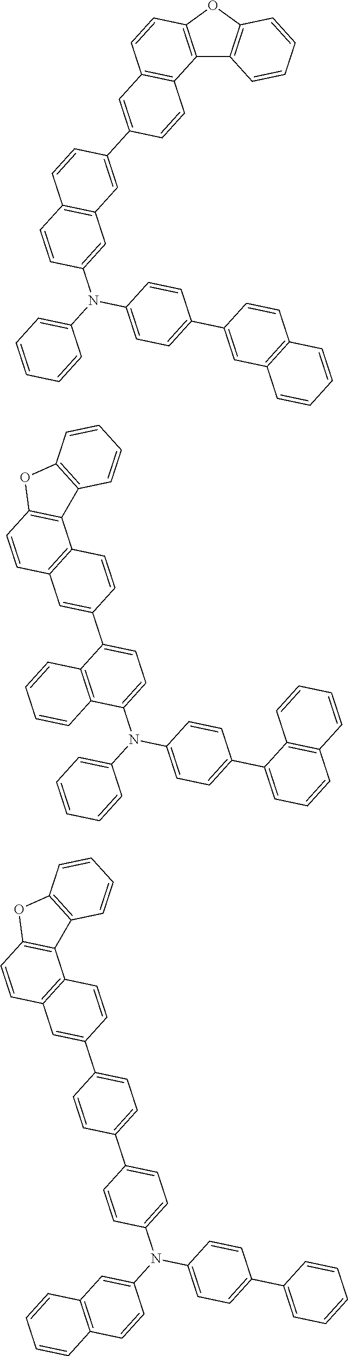

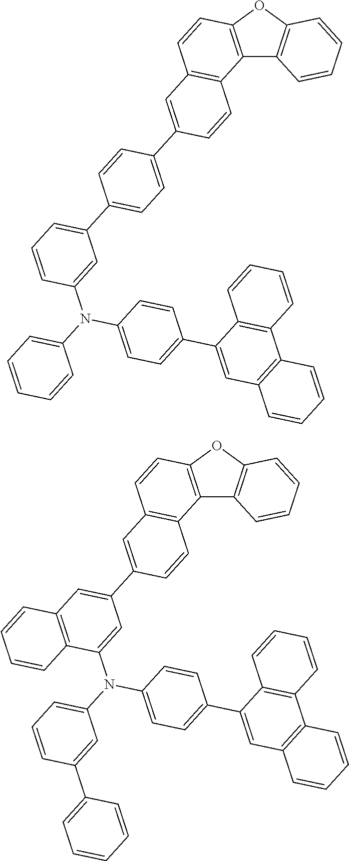

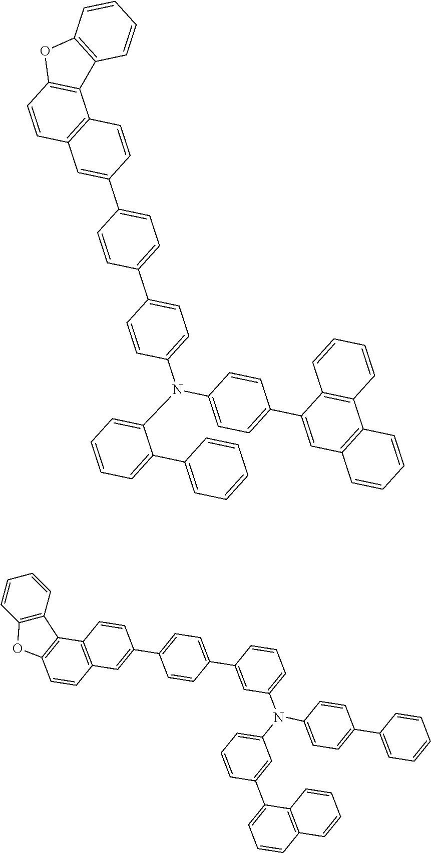 [SMILES: c1ccc(-c2ccccc2N(c2ccc(-c3ccc(-c4ccc5c(ccc6oc7ccccc7c65)c4)cc3)cc2)c2ccc(-c3cc4ccccc4c4ccccc34)cc2)cc1 ; c1ccc(-c2ccc(N(c3cccc(-c4ccc(-c5ccc6c(ccc7oc8ccccc8c76)c5)cc4)c3)c3cccc(-c4cccc5ccccc45)c3)cc2)cc1]

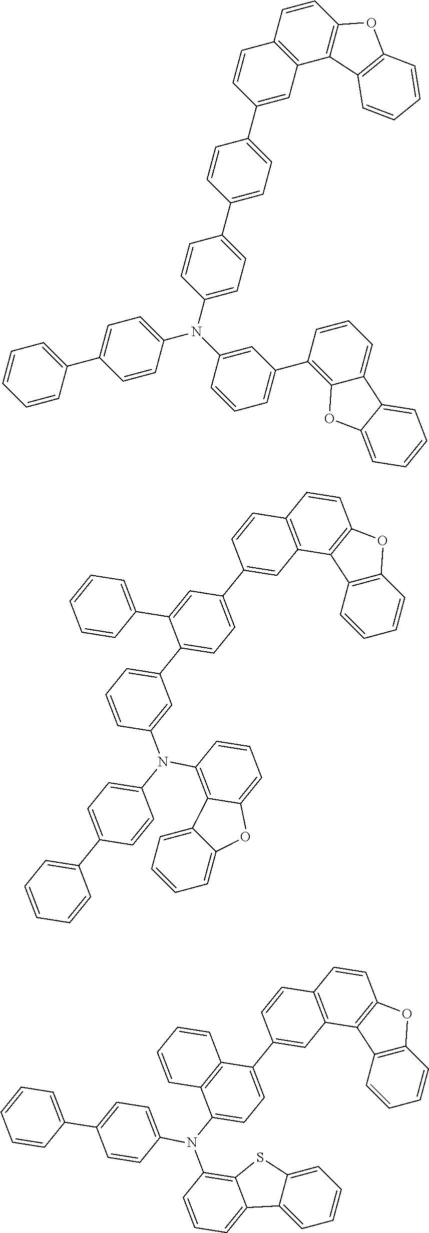 [SMILES: c1ccc(-c2ccc(N(c3ccc(-c4ccc(-c5ccc6ccc7oc8ccccc8c7c6c5)cc4)cc3)c3cccc(-c4cccc5c4oc4ccccc45)c3)cc2)cc1 ; c1ccc(-c2ccc(N(c3cccc(-c4ccc(-c5ccc6ccc7oc8ccccc8c7c6c5)cc4-c4ccccc4)c3)c3cccc4oc5ccccc5c34)cc2)cc1 ; c1ccc(-c2ccc(N(c3ccc(-c4ccc5ccc6oc7ccccc7c6c5c4)c4ccccc34)c3cccc4c3sc3ccccc34)cc2)cc1]

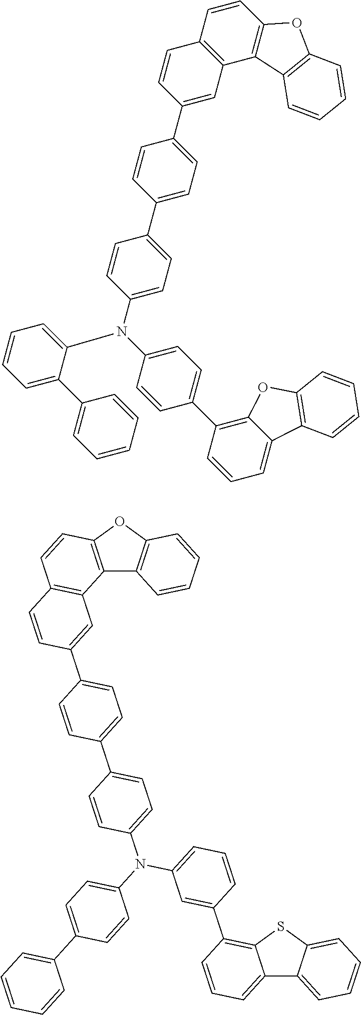 [SMILES: c1ccc(-c2ccccc2N(c2ccc(-c3ccc(-c4ccc5ccc6oc7ccccc7c6c5c4)cc3)cc2)c2ccc(-c3cccc4c3oc3ccccc34)cc2)cc1 ; c1ccc(-c2ccc(N(c3ccc(-c4ccc(-c5ccc6ccc7oc8ccccc8c7c6c5)cc4)cc3)c3cccc(-c4cccc5c4sc4ccccc45)c3)cc2)cc1]

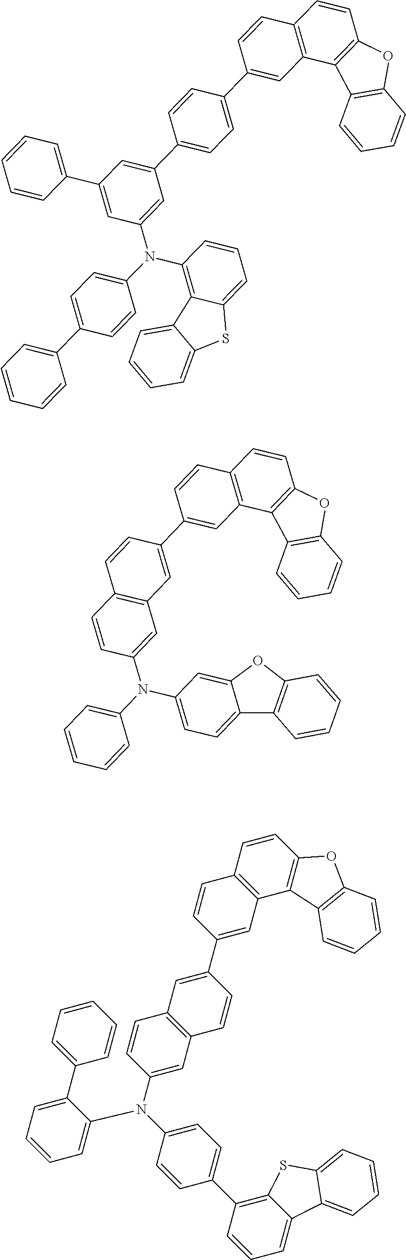 [SMILES: c1ccc(-c2ccc(N(c3cc(-c4ccccc4)cc(-c4ccc(-c5ccc6ccc7oc8ccccc8c7c6c5)cc4)c3)c3cccc4sc5ccccc5c34)cc2)cc1 ; c1ccc(N(c2ccc3ccc(-c4ccc5ccc6oc7ccccc7c6c5c4)cc3c2)c2ccc3c(c2)oc2ccccc23)cc1 ; c1ccc(-c2ccccc2N(c2ccc(-c3cccc4c3sc3ccccc34)cc2)c2ccc3cc(-c4ccc5ccc6oc7ccccc7c6c5c4)ccc3c2)cc1]

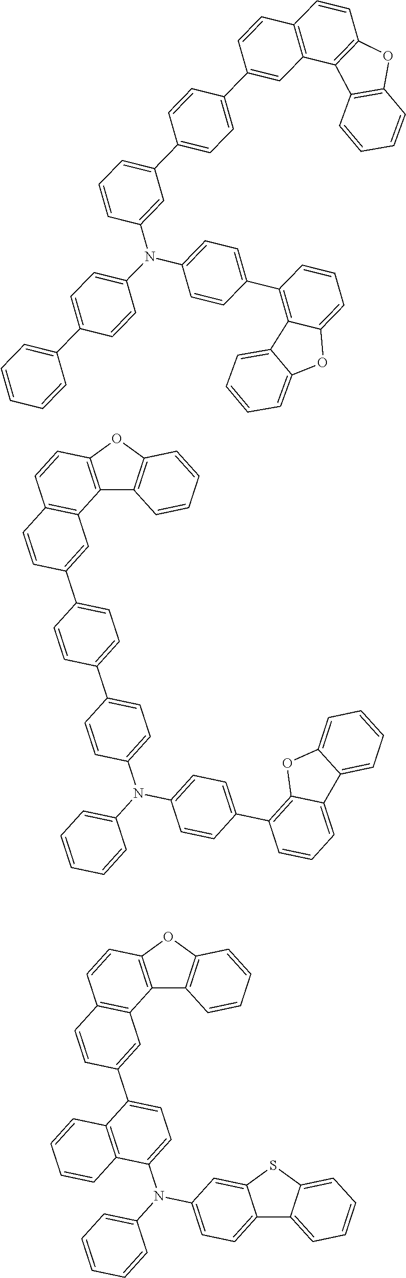

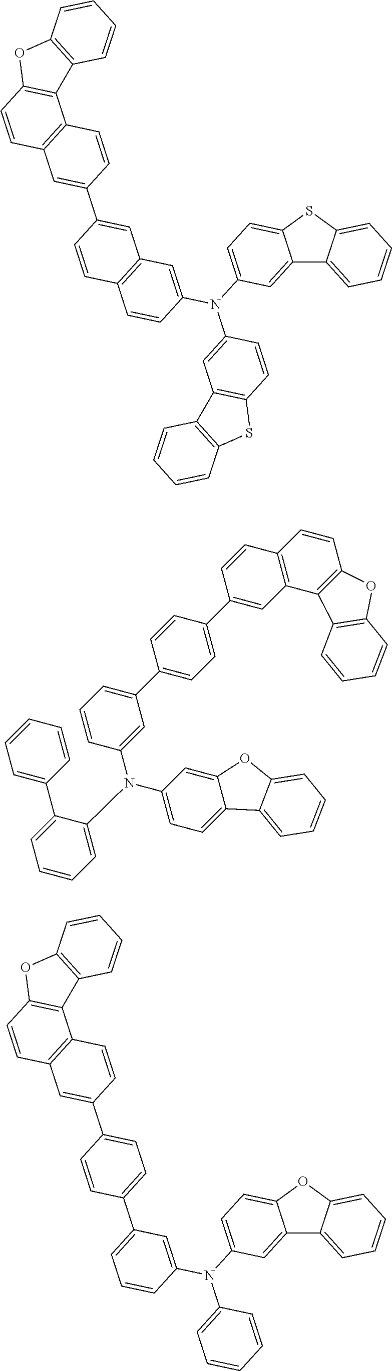

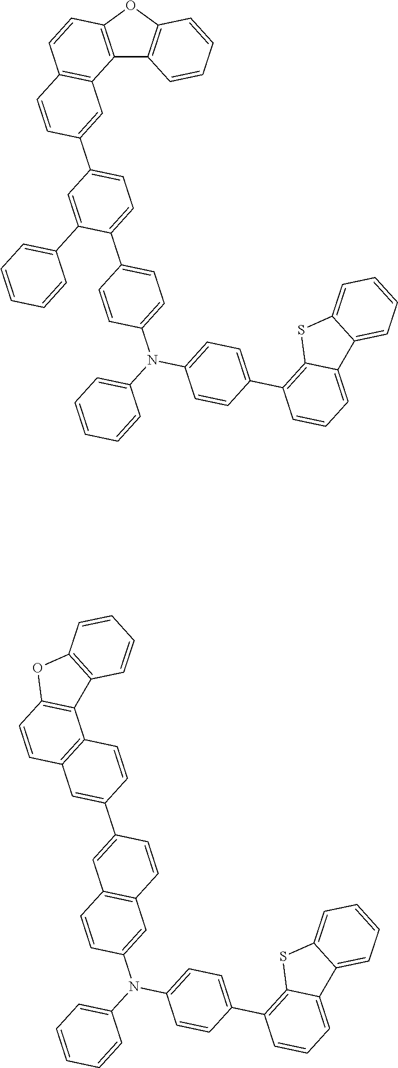 [SMILES: c1ccc(-c2cc(-c3ccc4ccc5oc6ccccc6c5c4c3)ccc2-c2ccc(N(c3ccccc3)c3ccc(-c4cccc5c4sc4ccccc45)cc3)cc2)cc1 ; c1ccc(N(c2ccc(-c3cccc4c3sc3ccccc34)cc2)c2ccc3cc(-c4ccc5c(ccc6oc7ccccc7c65)c4)ccc3c2)cc1]

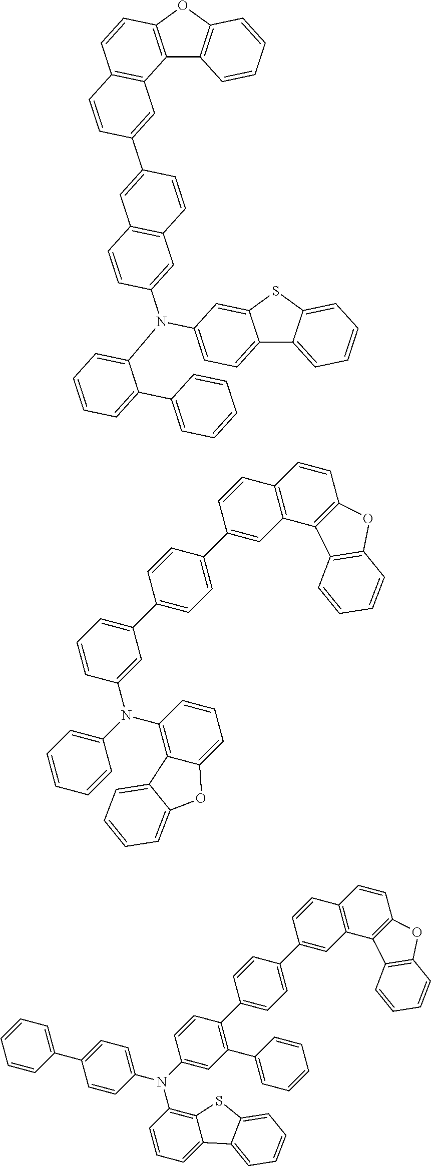 [SMILES: c1ccc(-c2ccccc2N(c2ccc3cc(-c4ccc5ccc6oc7ccccc7c6c5c4)ccc3c2)c2ccc3c(c2)sc2ccccc23)cc1 ; c1ccc(N(c2cccc(-c3ccc(-c4ccc5ccc6oc7ccccc7c6c5c4)cc3)c2)c2cccc3oc4ccccc4c23)cc1 ; c1ccc(-c2ccc(N(c3ccc(-c4ccc(-c5ccc6ccc7oc8ccccc8c7c6c5)cc4)c(-c4ccccc4)c3)c3cccc4c3sc3ccccc34)cc2)cc1]

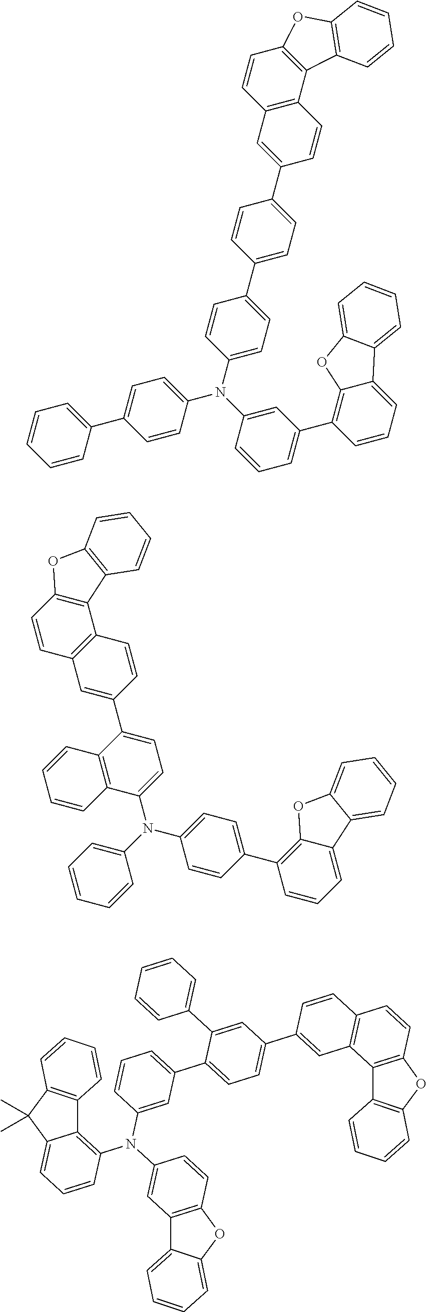 [SMILES: c1ccc(-c2ccc(N(c3ccc(-c4ccc(-c5ccc6c(ccc7oc8ccccc8c76)c5)cc4)cc3)c3cccc(-c4cccc5c4oc4ccccc45)c3)cc2)cc1 ; c1ccc(N(c2ccc(-c3cccc4c3oc3ccccc34)cc2)c2ccc(-c3ccc4c(ccc5oc6ccccc6c54)c3)c3ccccc23)cc1 ; CC1(C)c2ccccc2-c2c(N(c3cccc(-c4ccc(-c5ccc6ccc7oc8ccccc8c7c6c5)cc4-c4ccccc4)c3)c3ccc4oc5ccccc5c4c3)cccc21]

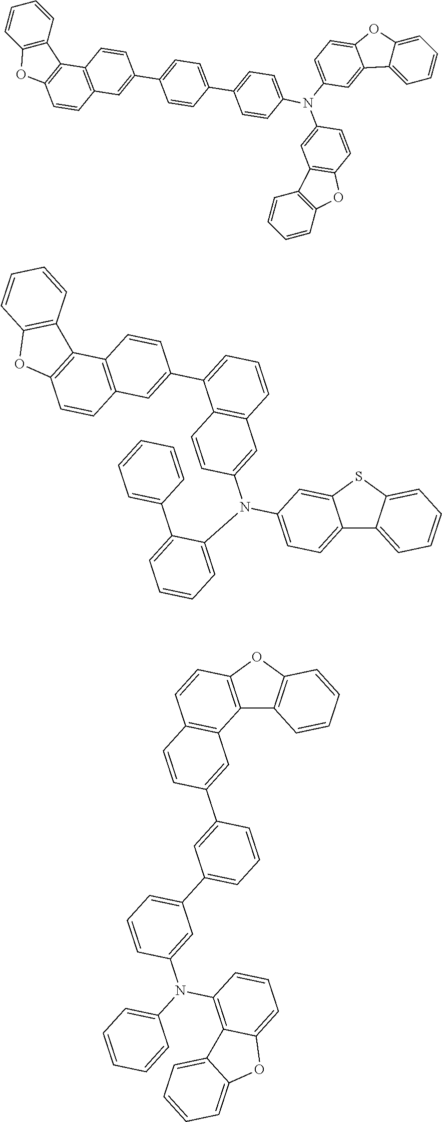 [SMILES: c1ccc2c(c1)oc1ccc(N(c3ccc(-c4ccc(-c5ccc6c(ccc7oc8ccccc8c76)c5)cc4)cc3)c3ccc4oc5ccccc5c4c3)cc12 ; c1ccc(-c2ccccc2N(c2ccc3c(-c4ccc5c(ccc6oc7ccccc7c65)c4)cccc3c2)c2ccc3c(c2)sc2ccccc23)cc1 ; c1ccc(N(c2cccc(-c3cccc(-c4ccc5ccc6oc7ccccc7c6c5c4)c3)c2)c2cccc3oc4ccccc4c23)cc1]

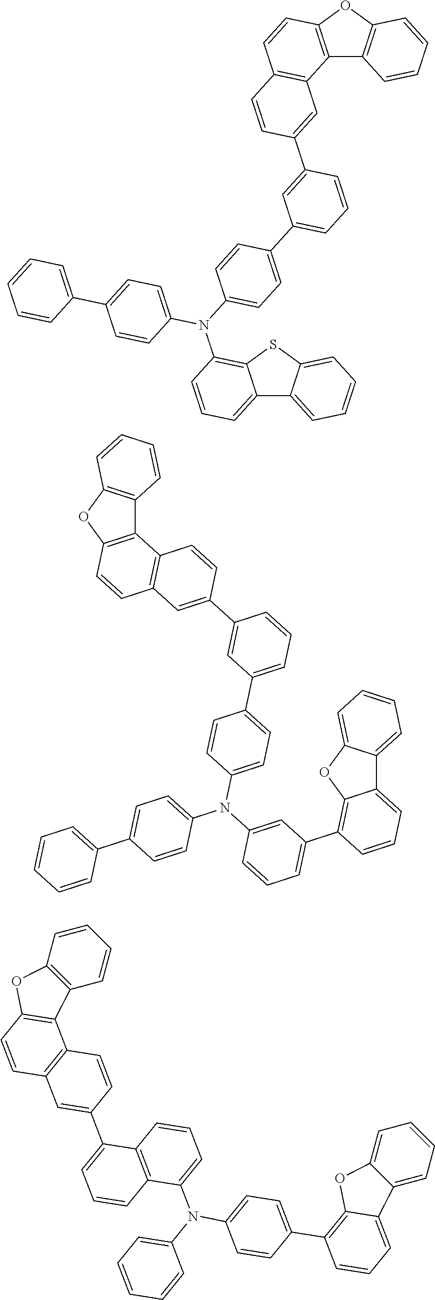

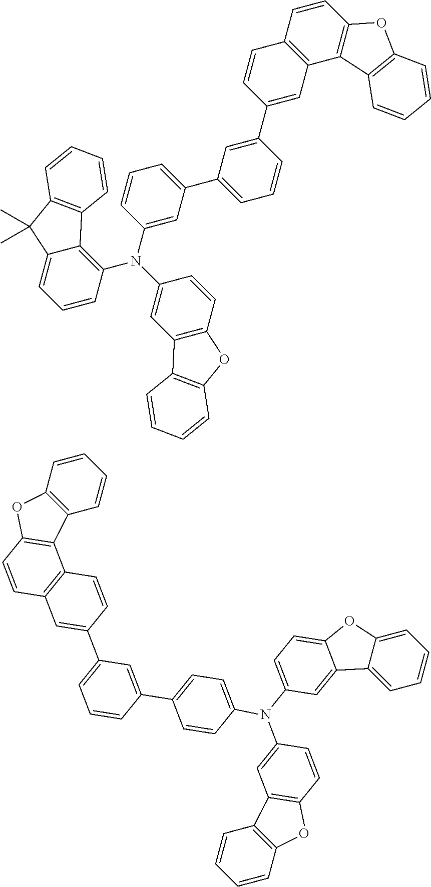 [SMILES: CC1(C)c2ccccc2-c2c(N(c3cccc(-c4cccc(-c5ccc6ccc7oc8ccccc8c7c6c5)c4)c3)c3ccc4oc5ccccc5c4c3)cccc21 ; c1cc(-c2ccc(N(c3ccc4oc5ccccc5c4c3)c3ccc4oc5ccccc5c4c3)cc2)cc(-c2ccc3c(ccc4oc5ccccc5c43)c2)c1]

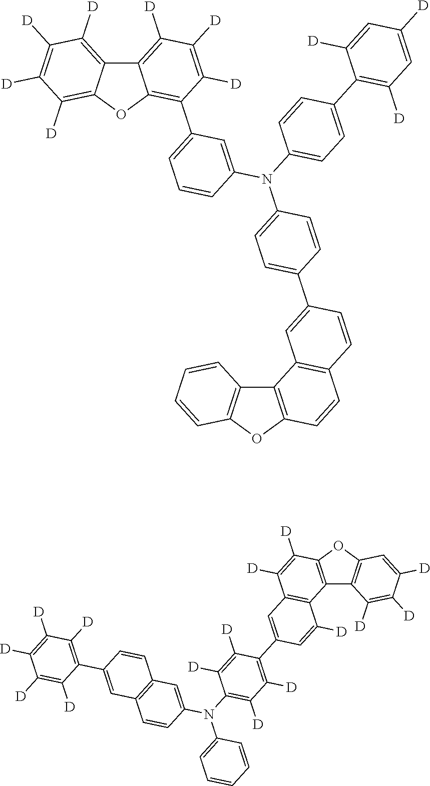 [SMILES: [2H]c1cc([2H])c(-c2ccc(N(c3ccc(-c4ccc5ccc6oc7ccccc7c6c5c4)cc3)c3cccc(-c4c([2H])c([2H])c([2H])c5c4oc4c([2H])c([2H])c([2H])c([2H])c45)c3)cc2)c([2H])c1 ; [2H]c1cc2oc3c([2H])c([2H])c4cc(-c5c([2H])c([2H])c(N(c6ccccc6)c6ccc7cc(-c8c([2H])c([2H])c([2H])c([2H])c8[2H])ccc7c6)c([2H])c5[2H])cc([2H])c4c3c2c([2H])c1[2H]]

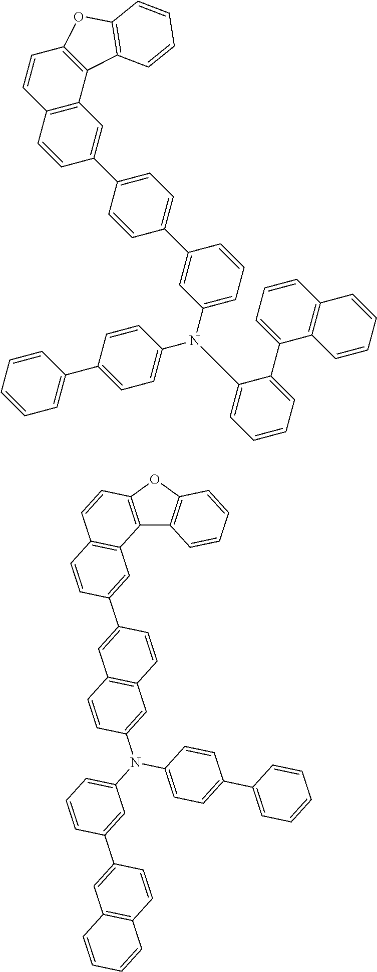 [SMILES: c1ccc(-c2ccc(N(c3cccc(-c4ccc(-c5ccc6ccc7oc8ccccc8c7c6c5)cc4)c3)c3ccccc3-c3cccc4ccccc34)cc2)cc1 ; c1ccc(-c2ccc(N(c3cccc(-c4ccc5ccccc5c4)c3)c3ccc4cc(-c5ccc6ccc7oc8ccccc8c7c6c5)ccc4c3)cc2)cc1]

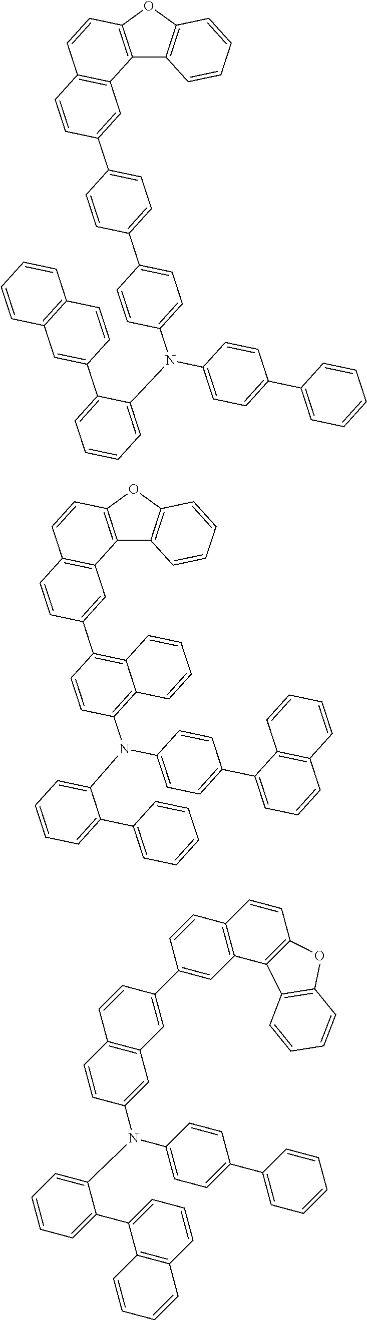

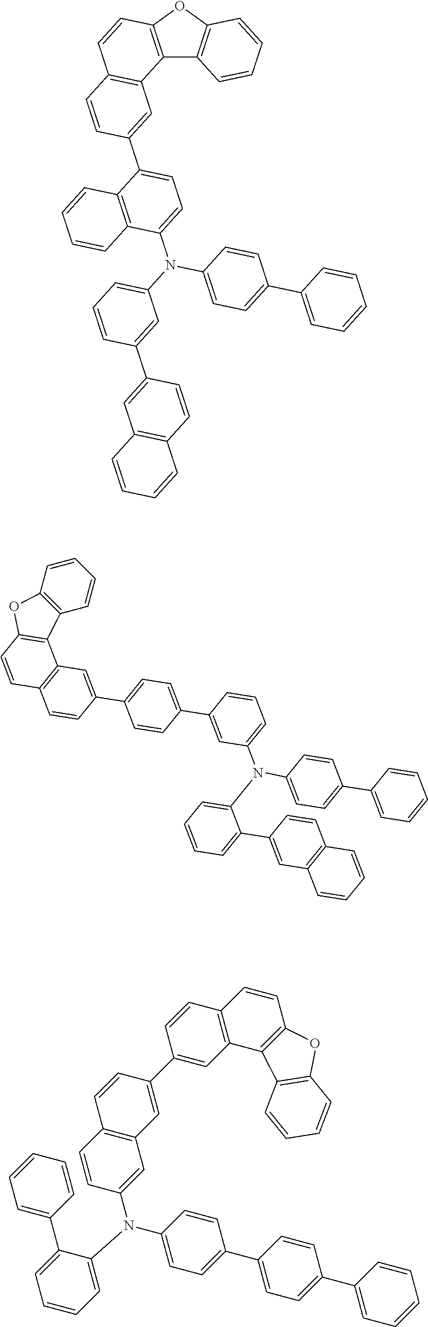 [SMILES: c1ccc(-c2ccc(N(c3cccc(-c4ccc5ccccc5c4)c3)c3ccc(-c4ccc5ccc6oc7ccccc7c6c5c4)c4ccccc34)cc2)cc1 ; c1ccc(-c2ccc(N(c3cccc(-c4ccc(-c5ccc6ccc7oc8ccccc8c7c6c5)cc4)c3)c3ccccc3-c3ccc4ccccc4c3)cc2)cc1 ; c1ccc(-c2ccc(-c3ccc(N(c4ccc5ccc(-c6ccc7ccc8oc9ccccc9c8c7c6)cc5c4)c4ccccc4-c4ccccc4)cc3)cc2)cc1]

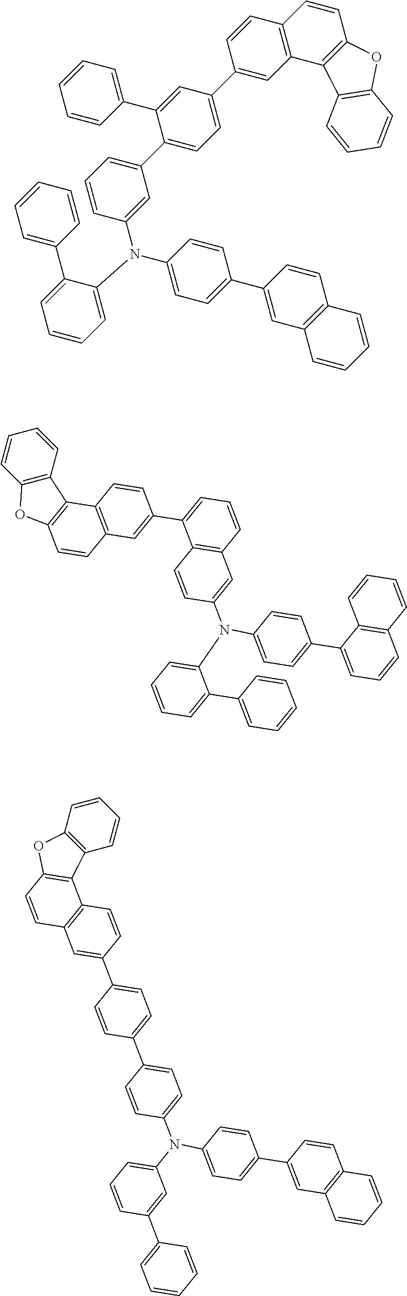 [SMILES: c1ccc(-c2cc(-c3ccc4ccc5oc6ccccc6c5c4c3)ccc2-c2cccc(N(c3ccc(-c4ccc5ccccc5c4)cc3)c3ccccc3-c3ccccc3)c2)cc1 ; c1ccc(-c2ccccc2N(c2ccc(-c3cccc4ccccc34)cc2)c2ccc3c(-c4ccc5c(ccc6oc7ccccc7c65)c4)cccc3c2)cc1 ; c1ccc(-c2cccc(N(c3ccc(-c4ccc(-c5ccc6c(ccc7oc8ccccc8c76)c5)cc4)cc3)c3ccc(-c4ccc5ccccc5c4)cc3)c2)cc1]

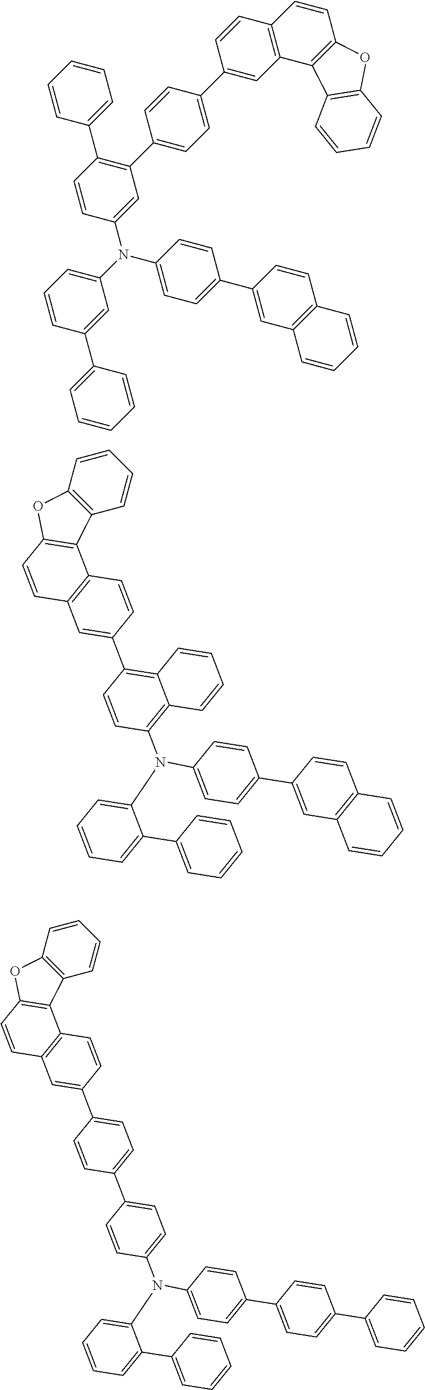

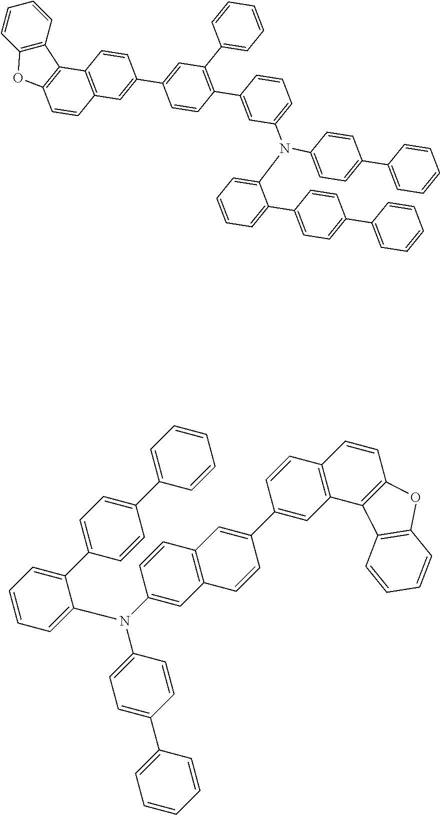 [SMILES: c1ccc(-c2ccc(-c3ccccc3N(c3ccc(-c4ccccc4)cc3)c3cccc(-c4ccc(-c5ccc6c(ccc7oc8ccccc8c76)c5)cc4-c4ccccc4)c3)cc2)cc1 ; c1ccc(-c2ccc(-c3ccccc3N(c3ccc(-c4ccccc4)cc3)c3ccc4cc(-c5ccc6ccc7oc8ccccc8c7c6c5)ccc4c3)cc2)cc1]

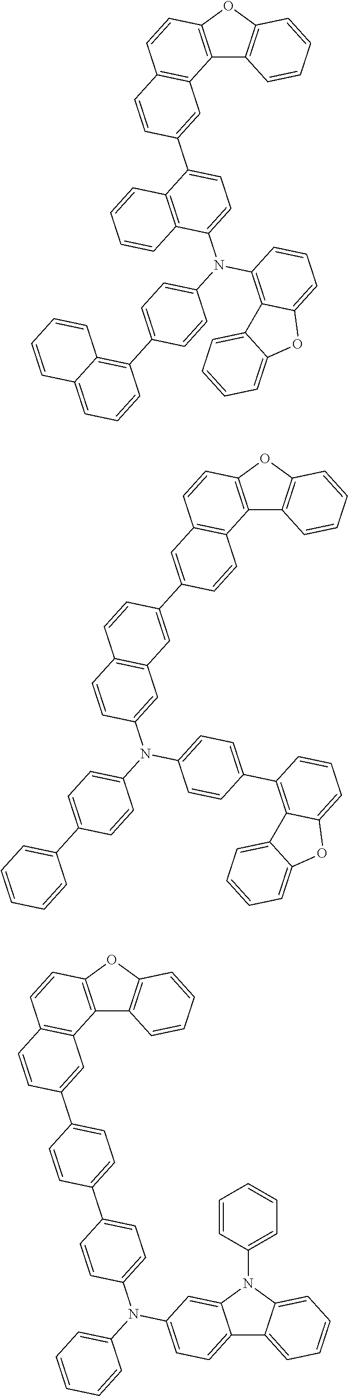 [SMILES: c1ccc2c(-c3ccc(N(c4ccc(-c5ccc6ccc7oc8ccccc8c7c6c5)c5ccccc45)c4cccc5oc6ccccc6c45)cc3)cccc2c1 ; c1ccc(-c2ccc(N(c3ccc(-c4cccc5oc6ccccc6c45)cc3)c3ccc4ccc(-c5ccc6c(ccc7oc8ccccc8c76)c5)cc4c3)cc2)cc1 ; c1ccc(N(c2ccc(-c3ccc(-c4ccc5ccc6oc7ccccc7c6c5c4)cc3)cc2)c2ccc3c4ccccc4n(-c4ccccc4)c3c2)cc1]

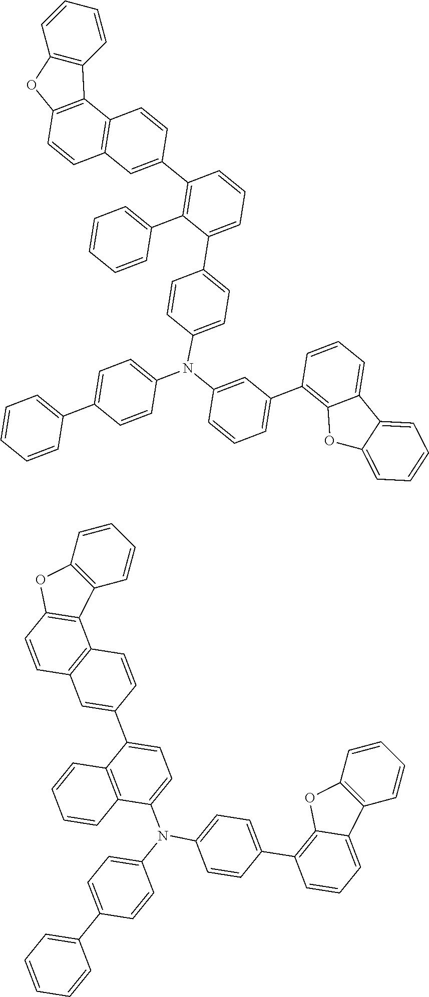 [SMILES: c1ccc(-c2ccc(N(c3ccc(-c4cccc(-c5ccc6c(ccc7oc8ccccc8c76)c5)c4-c4ccccc4)cc3)c3cccc(-c4cccc5c4oc4ccccc45)c3)cc2)cc1 ; c1ccc(-c2ccc(N(c3ccc(-c4cccc5c4oc4ccccc45)cc3)c3ccc(-c4ccc5c(ccc6oc7ccccc7c65)c4)c4ccccc34)cc2)cc1]

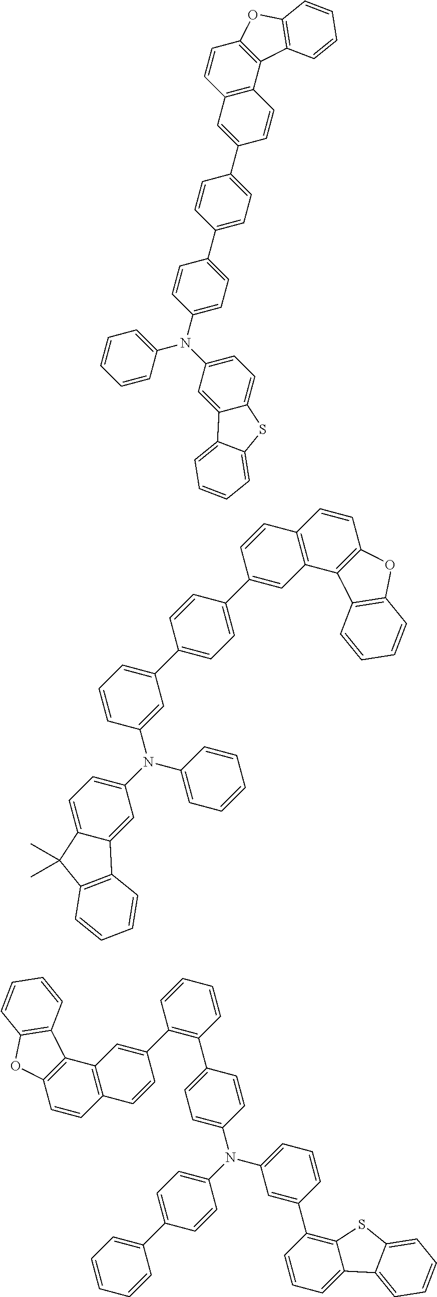 [SMILES: c1ccc(N(c2ccc(-c3ccc(-c4ccc5c(ccc6oc7ccccc7c65)c4)cc3)cc2)c2ccc3sc4ccccc4c3c2)cc1 ; CC1(C)c2ccccc2-c2cc(N(c3ccccc3)c3cccc(-c4ccc(-c5ccc6ccc7oc8ccccc8c7c6c5)cc4)c3)ccc21 ; c1ccc(-c2ccc(N(c3ccc(-c4ccccc4-c4ccc5ccc6oc7ccccc7c6c5c4)cc3)c3cccc(-c4cccc5c4sc4ccccc45)c3)cc2)cc1]

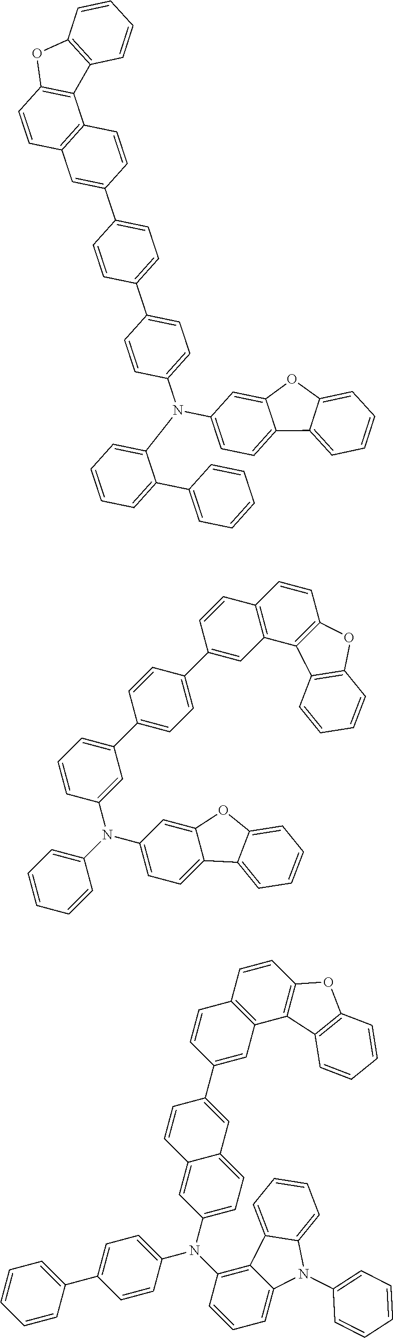 [SMILES: c1ccc(-c2ccccc2N(c2ccc(-c3ccc(-c4ccc5c(ccc6oc7ccccc7c65)c4)cc3)cc2)c2ccc3c(c2)oc2ccccc23)cc1 ; c1ccc(N(c2cccc(-c3ccc(-c4ccc5ccc6oc7ccccc7c6c5c4)cc3)c2)c2ccc3c(c2)oc2ccccc23)cc1 ; c1ccc(-c2ccc(N(c3ccc4cc(-c5ccc6ccc7oc8ccccc8c7c6c5)ccc4c3)c3cccc4c3c3ccccc3n4-c3ccccc3)cc2)cc1]

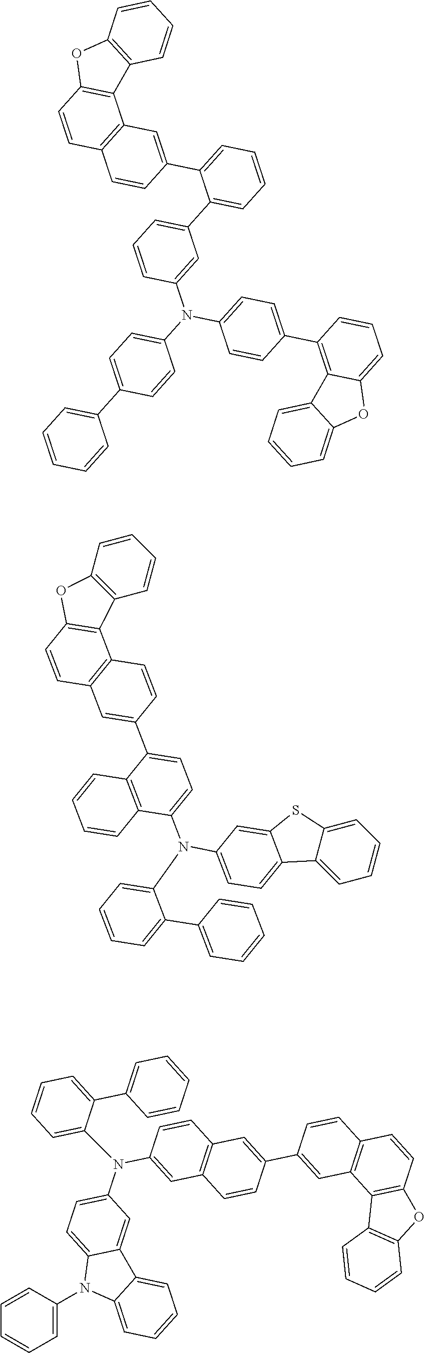 [SMILES: c1ccc(-c2ccc(N(c3ccc(-c4cccc5oc6ccccc6c45)cc3)c3cccc(-c4ccccc4-c4ccc5ccc6oc7ccccc7c6c5c4)c3)cc2)cc1 ; c1ccc(-c2ccccc2N(c2ccc3c(c2)sc2ccccc23)c2ccc(-c3ccc4c(ccc5oc6ccccc6c54)c3)c3ccccc23)cc1 ; c1ccc(-c2ccccc2N(c2ccc3cc(-c4ccc5ccc6oc7ccccc7c6c5c4)ccc3c2)c2ccc3c(c2)c2ccccc2n3-c2ccccc2)cc1]

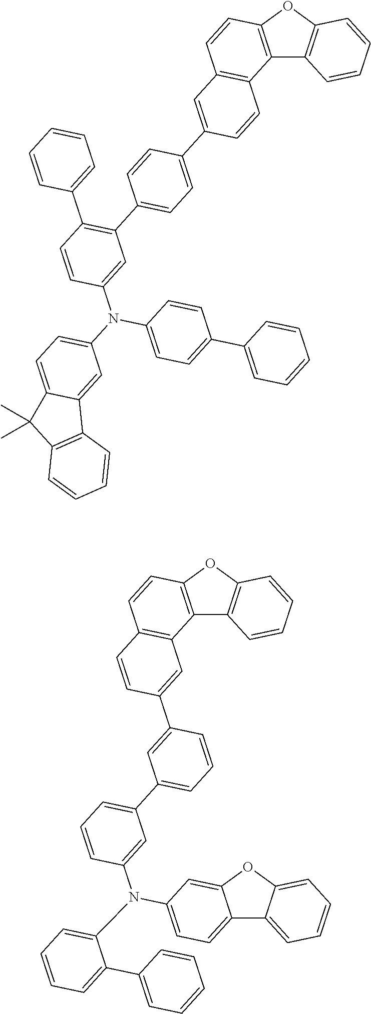 [SMILES: CC1(C)c2ccccc2-c2cc(N(c3ccc(-c4ccccc4)cc3)c3ccc(-c4ccccc4)c(-c4ccc(-c5ccc6c(ccc7oc8ccccc8c76)c5)cc4)c3)ccc21 ; c1ccc(-c2ccccc2N(c2cccc(-c3cccc(-c4ccc5ccc6oc7ccccc7c6c5c4)c3)c2)c2ccc3c(c2)oc2ccccc23)cc1]

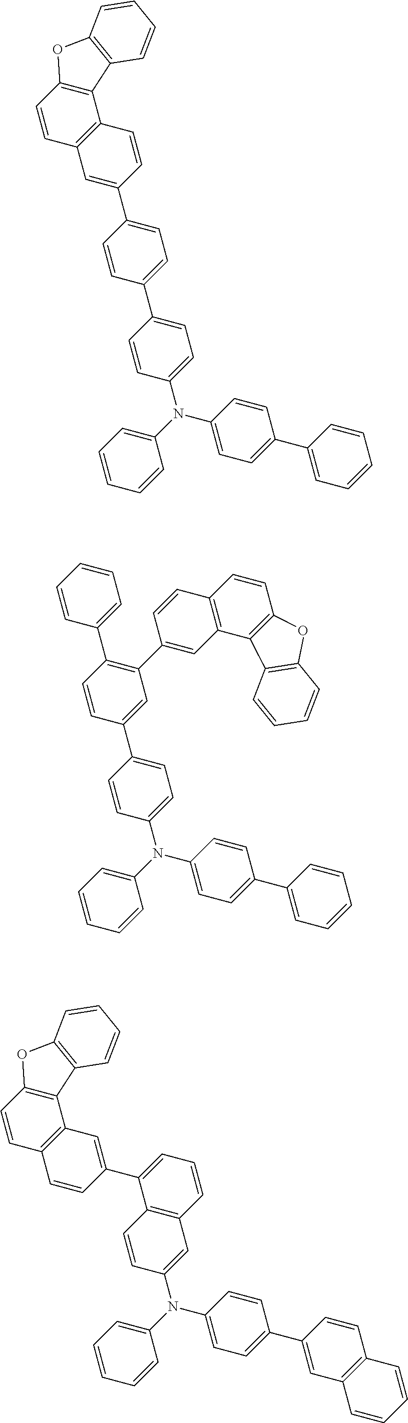 [SMILES: c1ccc(-c2ccc(N(c3ccccc3)c3ccc(-c4ccc(-c5ccc6c(ccc7oc8ccccc8c76)c5)cc4)cc3)cc2)cc1 ; c1ccc(-c2ccc(N(c3ccccc3)c3ccc(-c4ccc(-c5ccccc5)c(-c5ccc6ccc7oc8ccccc8c7c6c5)c4)cc3)cc2)cc1 ; c1ccc(N(c2ccc(-c3ccc4ccccc4c3)cc2)c2ccc3c(-c4ccc5ccc6oc7ccccc7c6c5c4)cccc3c2)cc1]

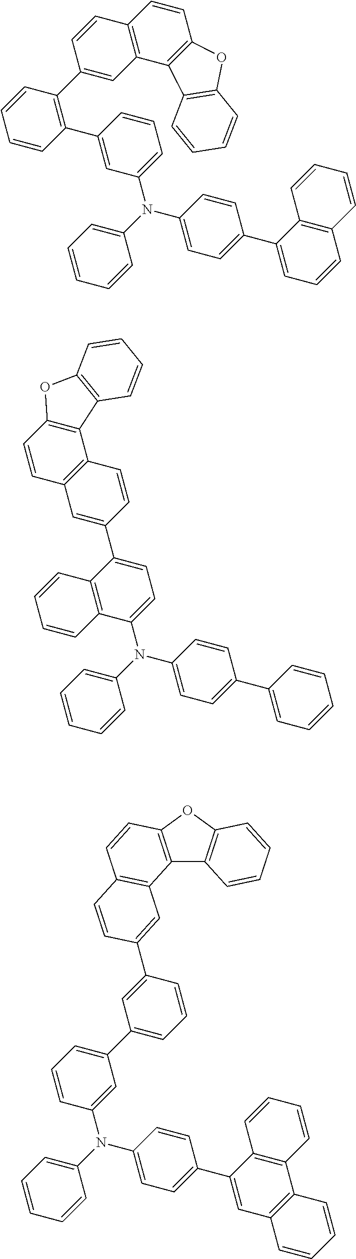 [SMILES: c1ccc(N(c2ccc(-c3cccc4ccccc34)cc2)c2cccc(-c3ccccc3-c3ccc4ccc5oc6ccccc6c5c4c3)c2)cc1 ; c1ccc(-c2ccc(N(c3ccccc3)c3ccc(-c4ccc5c(ccc6oc7ccccc7c65)c4)c4ccccc34)cc2)cc1 ; c1ccc(N(c2ccc(-c3cc4ccccc4c4ccccc34)cc2)c2cccc(-c3cccc(-c4ccc5ccc6oc7ccccc7c6c5c4)c3)c2)cc1]

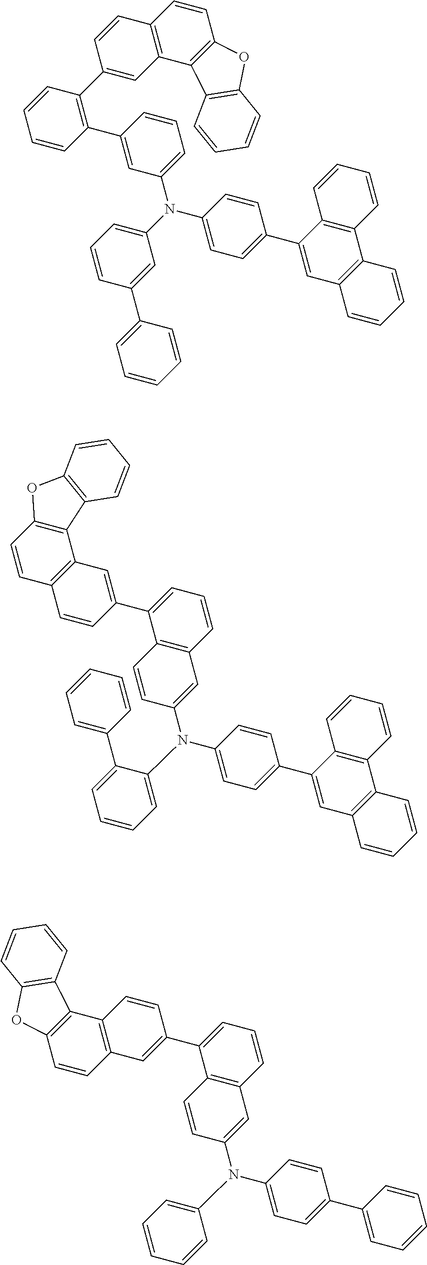 [SMILES: c1ccc(-c2cccc(N(c3ccc(-c4cc5ccccc5c5ccccc45)cc3)c3cccc(-c4ccccc4-c4ccc5ccc6oc7ccccc7c6c5c4)c3)c2)cc1 ; c1ccc(-c2ccccc2N(c2ccc(-c3cc4ccccc4c4ccccc34)cc2)c2ccc3c(-c4ccc5ccc6oc7ccccc7c6c5c4)cccc3c2)cc1 ; c1ccc(-c2ccc(N(c3ccccc3)c3ccc4c(-c5ccc6c(ccc7oc8ccccc8c76)c5)cccc4c3)cc2)cc1]

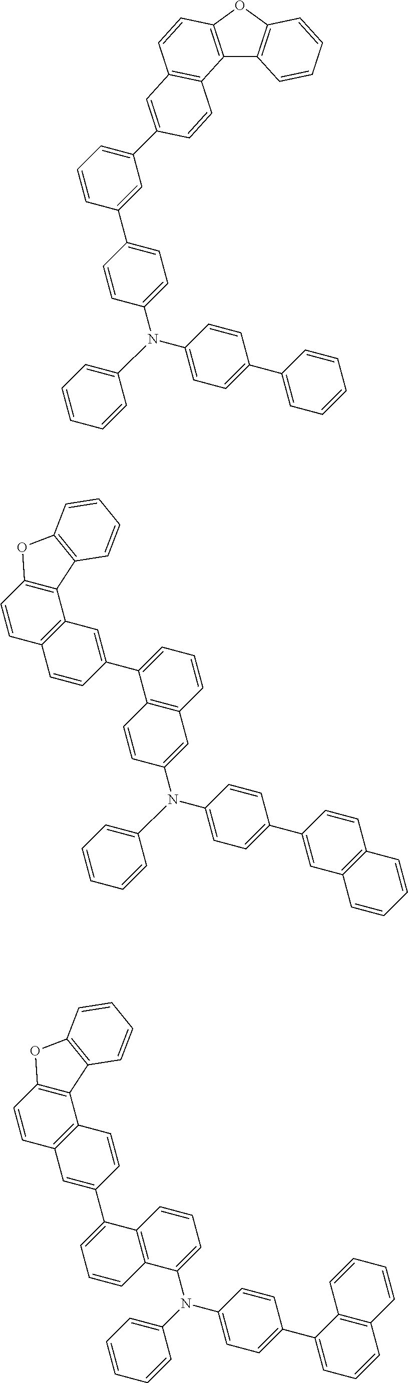 [SMILES: c1ccc(-c2ccc(N(c3ccccc3)c3ccc(-c4cccc(-c5ccc6c(ccc7oc8ccccc8c76)c5)c4)cc3)cc2)cc1 ; c1ccc(N(c2ccc(-c3ccc4ccccc4c3)cc2)c2ccc3c(-c4ccc5ccc6oc7ccccc7c6c5c4)cccc3c2)cc1 ; c1ccc(N(c2ccc(-c3cccc4ccccc34)cc2)c2cccc3c(-c4ccc5c(ccc6oc7ccccc7c65)c4)cccc23)cc1]

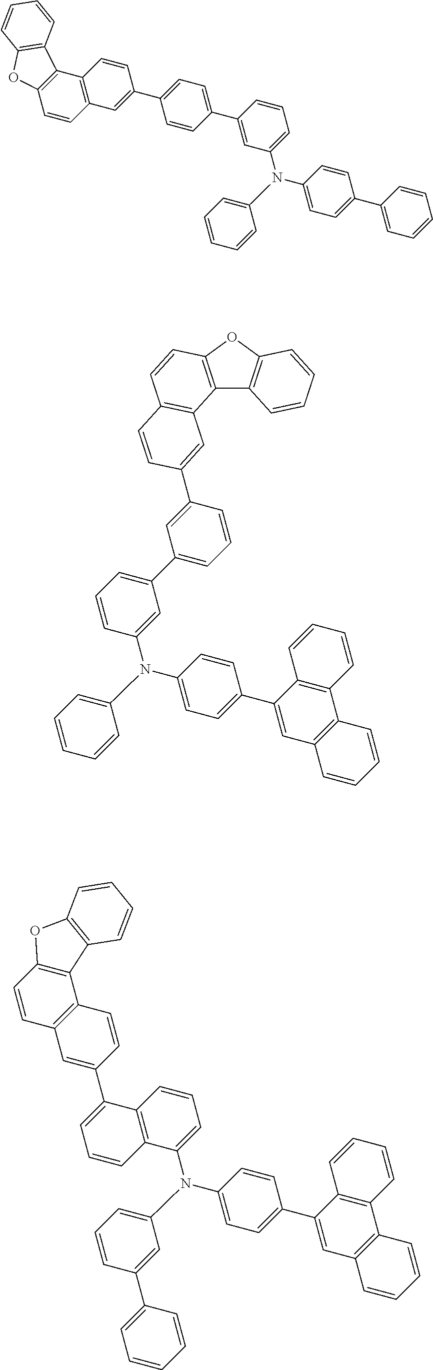 [SMILES: c1ccc(-c2ccc(N(c3ccccc3)c3cccc(-c4ccc(-c5ccc6c(ccc7oc8ccccc8c76)c5)cc4)c3)cc2)cc1 ; c1ccc(N(c2ccc(-c3cc4ccccc4c4ccccc34)cc2)c2cccc(-c3cccc(-c4ccc5ccc6oc7ccccc7c6c5c4)c3)c2)cc1 ; c1ccc(-c2cccc(N(c3ccc(-c4cc5ccccc5c5ccccc45)cc3)c3cccc4c(-c5ccc6c(ccc7oc8ccccc8c76)c5)cccc34)c2)cc1]

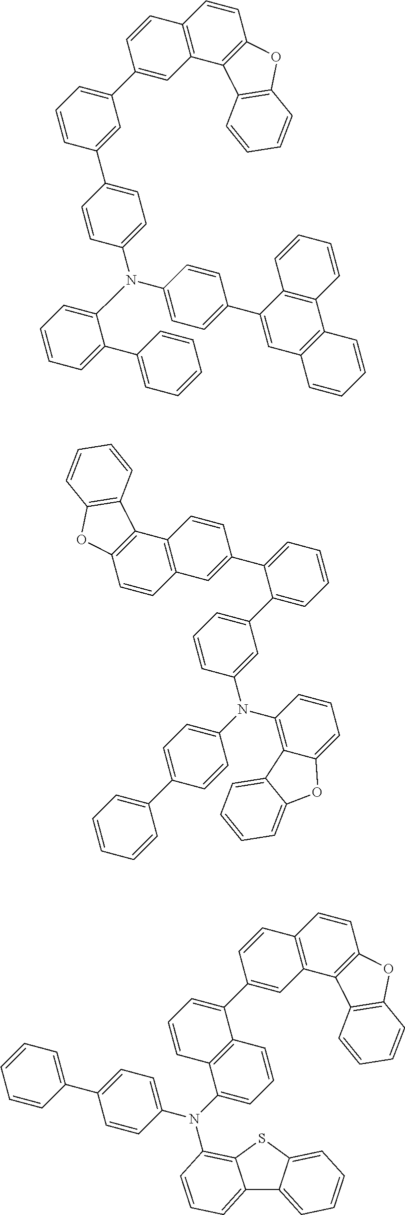 [SMILES: c1ccc(-c2ccccc2N(c2ccc(-c3cccc(-c4ccc5ccc6oc7ccccc7c6c5c4)c3)cc2)c2ccc(-c3cc4ccccc4c4ccccc34)cc2)cc1 ; c1ccc(-c2ccc(N(c3cccc(-c4ccccc4-c4ccc5c(ccc6oc7ccccc7c65)c4)c3)c3cccc4oc5ccccc5c34)cc2)cc1 ; c1ccc(-c2ccc(N(c3cccc4c(-c5ccc6ccc7oc8ccccc8c7c6c5)cccc34)c3cccc4c3sc3ccccc34)cc2)cc1]

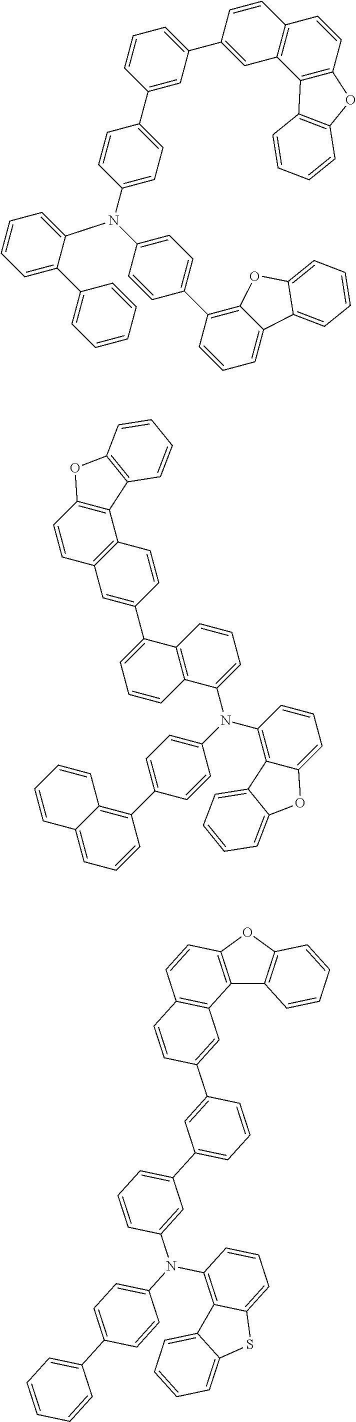 [SMILES: c1ccc(-c2ccccc2N(c2ccc(-c3cccc(-c4ccc5ccc6oc7ccccc7c6c5c4)c3)cc2)c2ccc(-c3cccc4c3oc3ccccc34)cc2)cc1 ; c1ccc2c(-c3ccc(N(c4cccc5c(-c6ccc7c(ccc8oc9ccccc9c87)c6)cccc45)c4cccc5oc6ccccc6c45)cc3)cccc2c1 ; c1ccc(-c2ccc(N(c3cccc(-c4cccc(-c5ccc6ccc7oc8ccccc8c7c6c5)c4)c3)c3cccc4sc5ccccc5c34)cc2)cc1]

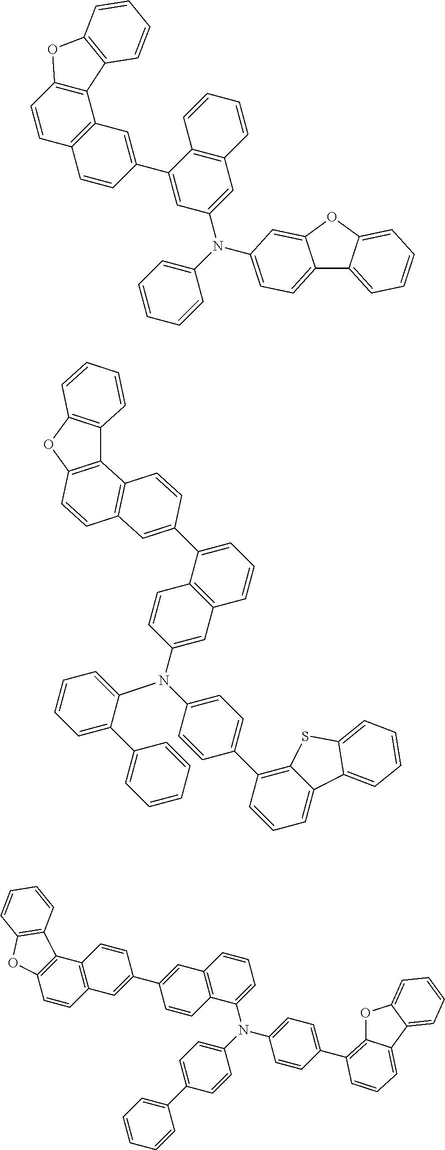 [SMILES: c1ccc(N(c2cc(-c3ccc4ccc5oc6ccccc6c5c4c3)c3ccccc3c2)c2ccc3c(c2)oc2ccccc23)cc1 ; c1ccc(-c2ccccc2N(c2ccc(-c3cccc4c3sc3ccccc34)cc2)c2ccc3c(-c4ccc5c(ccc6oc7ccccc7c65)c4)cccc3c2)cc1 ; c1ccc(-c2ccc(N(c3ccc(-c4cccc5c4oc4ccccc45)cc3)c3cccc4cc(-c5ccc6c(ccc7oc8ccccc8c76)c5)ccc34)cc2)cc1]

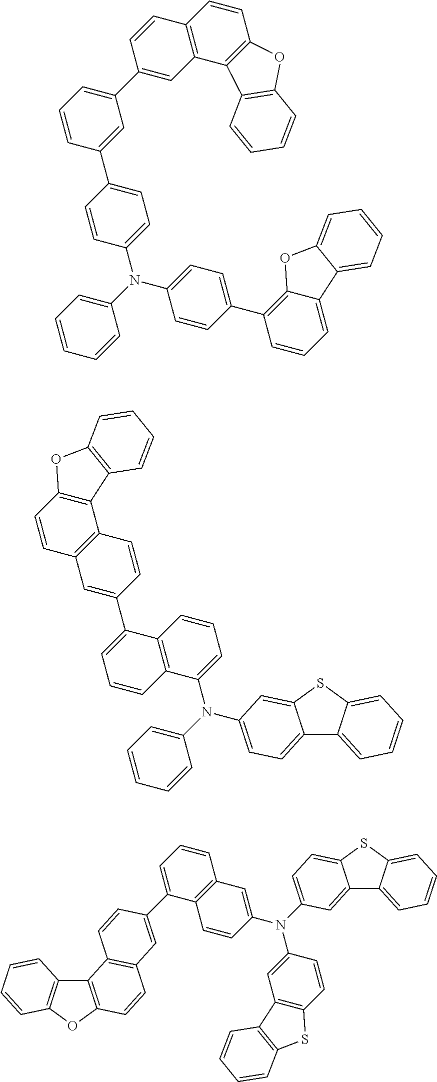 [SMILES: c1ccc(N(c2ccc(-c3cccc(-c4ccc5ccc6oc7ccccc7c6c5c4)c3)cc2)c2ccc(-c3cccc4c3oc3ccccc34)cc2)cc1 ; c1ccc(N(c2ccc3c(c2)sc2ccccc23)c2cccc3c(-c4ccc5c(ccc6oc7ccccc7c65)c4)cccc23)cc1 ; c1cc(-c2ccc3c(ccc4oc5ccccc5c43)c2)c2ccc(N(c3ccc4sc5ccccc5c4c3)c3ccc4sc5ccccc5c4c3)cc2c1]

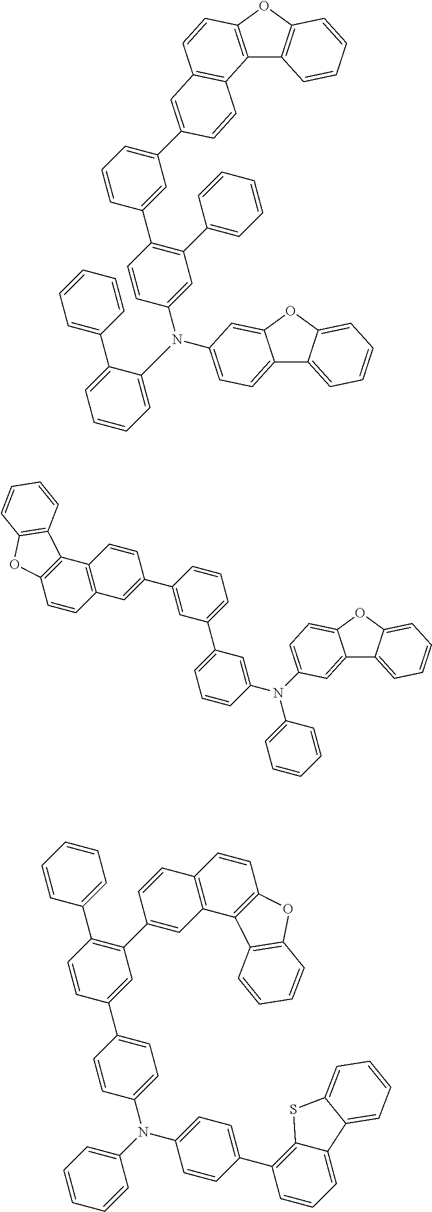 [SMILES: c1ccc(-c2cc(N(c3ccc4c(c3)oc3ccccc34)c3ccccc3-c3ccccc3)ccc2-c2cccc(-c3ccc4c(ccc5oc6ccccc6c54)c3)c2)cc1 ; c1ccc(N(c2cccc(-c3cccc(-c4ccc5c(ccc6oc7ccccc7c65)c4)c3)c2)c2ccc3oc4ccccc4c3c2)cc1 ; c1ccc(-c2ccc(-c3ccc(N(c4ccccc4)c4ccc(-c5cccc6c5sc5ccccc56)cc4)cc3)cc2-c2ccc3ccc4oc5ccccc5c4c3c2)cc1]

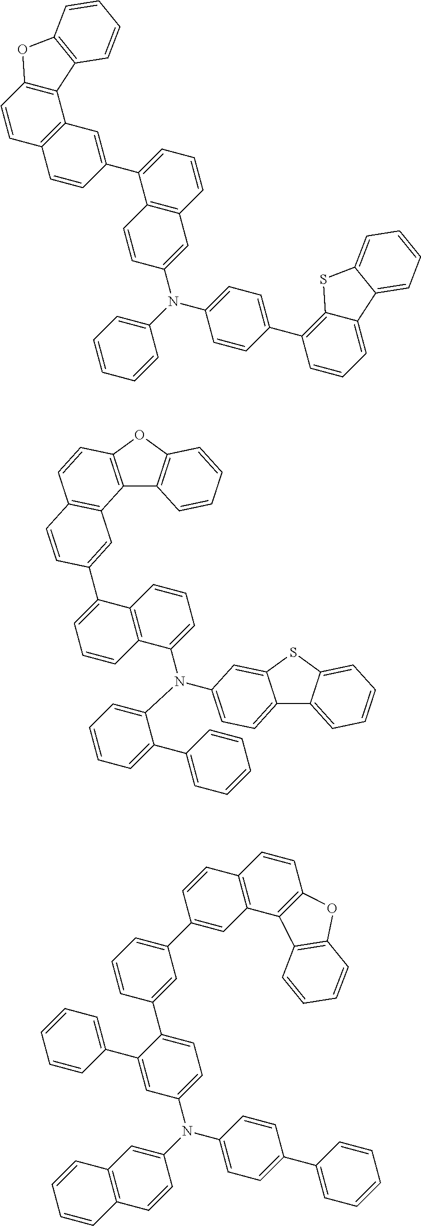 [SMILES: c1ccc(N(c2ccc(-c3cccc4c3sc3ccccc34)cc2)c2ccc3c(-c4ccc5ccc6oc7ccccc7c6c5c4)cccc3c2)cc1 ; c1ccc(-c2ccccc2N(c2ccc3c(c2)sc2ccccc23)c2cccc3c(-c4ccc5ccc6oc7ccccc7c6c5c4)cccc23)cc1 ; c1ccc(-c2ccc(N(c3ccc(-c4cccc(-c5ccc6ccc7oc8ccccc8c7c6c5)c4)c(-c4ccccc4)c3)c3ccc4ccccc4c3)cc2)cc1]

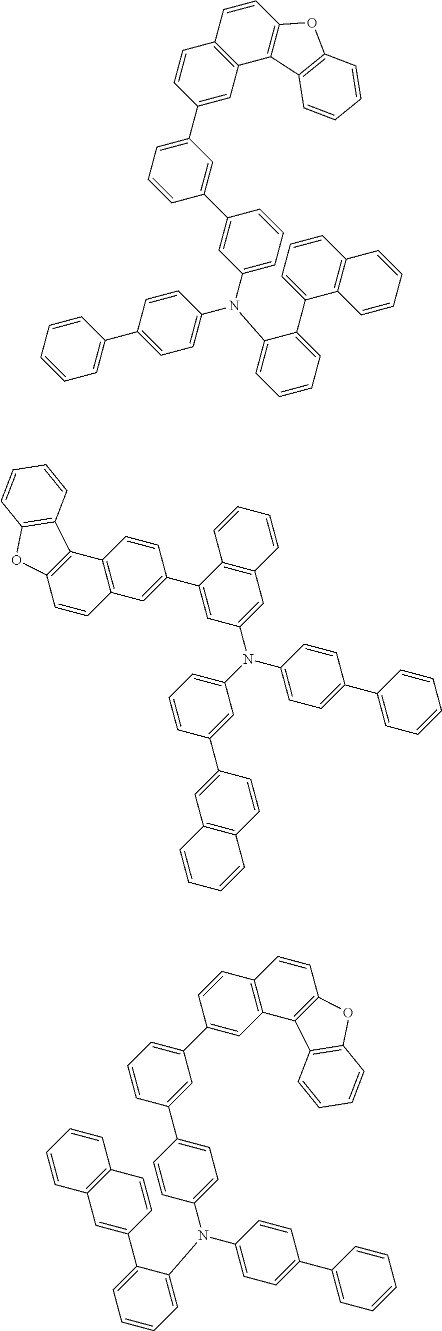 [SMILES: c1ccc(-c2ccc(N(c3cccc(-c4cccc(-c5ccc6ccc7oc8ccccc8c7c6c5)c4)c3)c3ccccc3-c3cccc4ccccc34)cc2)cc1 ; c1ccc(-c2ccc(N(c3cccc(-c4ccc5ccccc5c4)c3)c3cc(-c4ccc5c(ccc6oc7ccccc7c65)c4)c4ccccc4c3)cc2)cc1 ; c1ccc(-c2ccc(N(c3ccc(-c4cccc(-c5ccc6ccc7oc8ccccc8c7c6c5)c4)cc3)c3ccccc3-c3ccc4ccccc4c3)cc2)cc1]

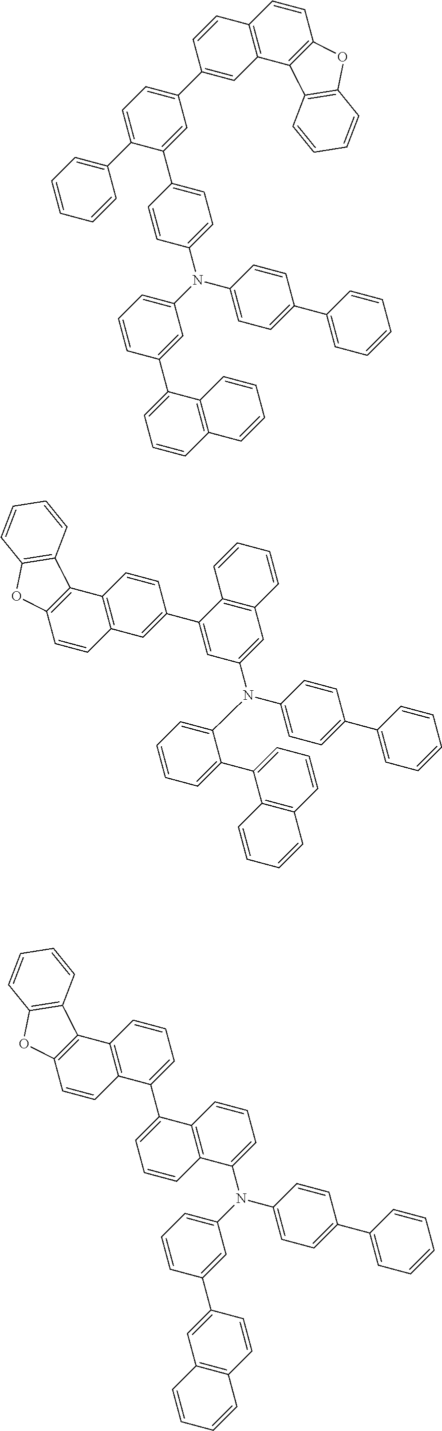 [SMILES: c1ccc(-c2ccc(N(c3ccc(-c4cc(-c5ccc6ccc7oc8ccccc8c7c6c5)ccc4-c4ccccc4)cc3)c3cccc(-c4cccc5ccccc45)c3)cc2)cc1 ; c1ccc(-c2ccc(N(c3cc(-c4ccc5c(ccc6oc7ccccc7c65)c4)c4ccccc4c3)c3ccccc3-c3cccc4ccccc34)cc2)cc1 ; c1ccc(-c2ccc(N(c3cccc(-c4ccc5ccccc5c4)c3)c3cccc4c(-c5cccc6c5ccc5oc7ccccc7c56)cccc34)cc2)cc1]

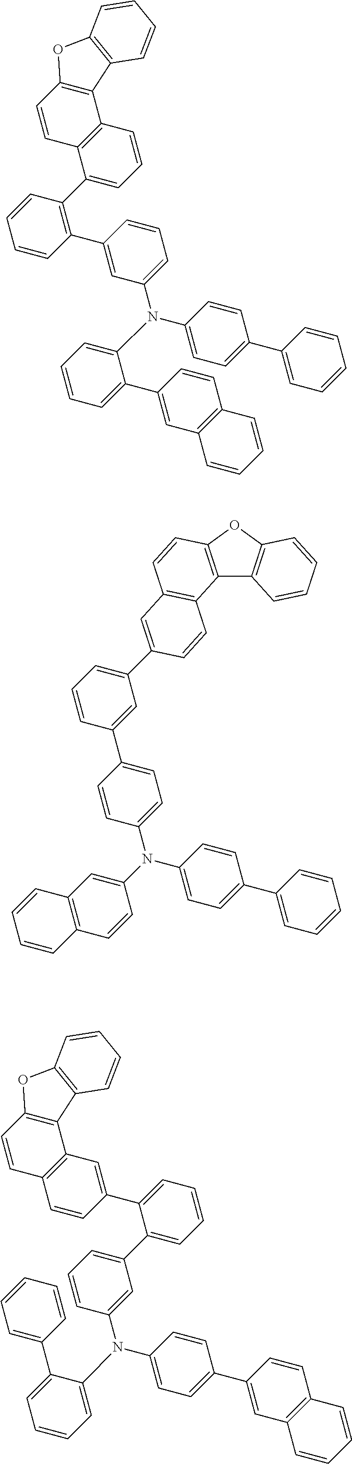 [SMILES: c1ccc(-c2ccc(N(c3cccc(-c4ccccc4-c4cccc5c4ccc4oc6ccccc6c45)c3)c3ccccc3-c3ccc4ccccc4c3)cc2)cc1 ; c1ccc(-c2ccc(N(c3ccc(-c4cccc(-c5ccc6c(ccc7oc8ccccc8c76)c5)c4)cc3)c3ccc4ccccc4c3)cc2)cc1 ; c1ccc(-c2ccccc2N(c2ccc(-c3ccc4ccccc4c3)cc2)c2cccc(-c3ccccc3-c3ccc4ccc5oc6ccccc6c5c4c3)c2)cc1]

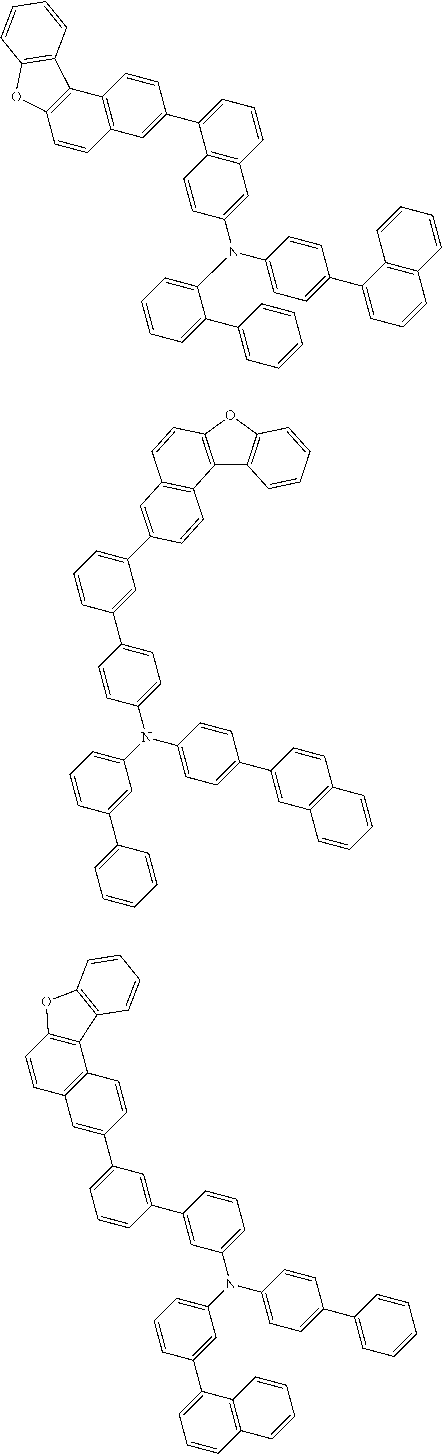 [SMILES: c1ccc(-c2ccccc2N(c2ccc(-c3cccc4ccccc34)cc2)c2ccc3c(-c4ccc5c(ccc6oc7ccccc7c65)c4)cccc3c2)cc1 ; c1ccc(-c2cccc(N(c3ccc(-c4cccc(-c5ccc6c(ccc7oc8ccccc8c76)c5)c4)cc3)c3ccc(-c4ccc5ccccc5c4)cc3)c2)cc1 ; c1ccc(-c2ccc(N(c3cccc(-c4cccc(-c5ccc6c(ccc7oc8ccccc8c76)c5)c4)c3)c3cccc(-c4cccc5ccccc45)c3)cc2)cc1]

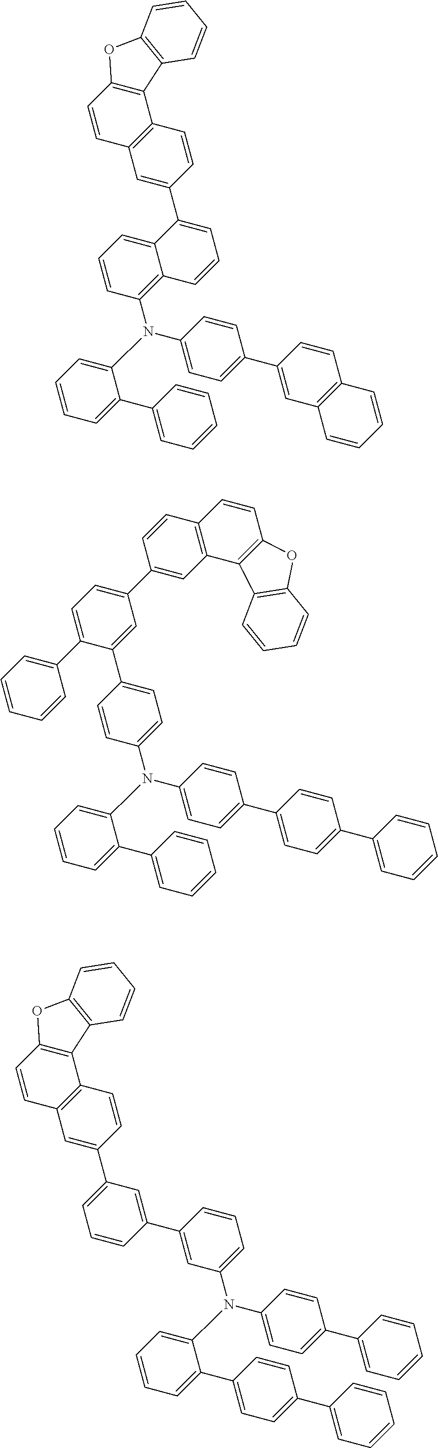 [SMILES: c1ccc(-c2ccccc2N(c2ccc(-c3ccc4ccccc4c3)cc2)c2cccc3c(-c4ccc5c(ccc6oc7ccccc7c65)c4)cccc23)cc1 ; c1ccc(-c2ccc(-c3ccc(N(c4ccc(-c5cc(-c6ccc7ccc8oc9ccccc9c8c7c6)ccc5-c5ccccc5)cc4)c4ccccc4-c4ccccc4)cc3)cc2)cc1 ; c1ccc(-c2ccc(-c3ccccc3N(c3ccc(-c4ccccc4)cc3)c3cccc(-c4cccc(-c5ccc6c(ccc7oc8ccccc8c76)c5)c4)c3)cc2)cc1]

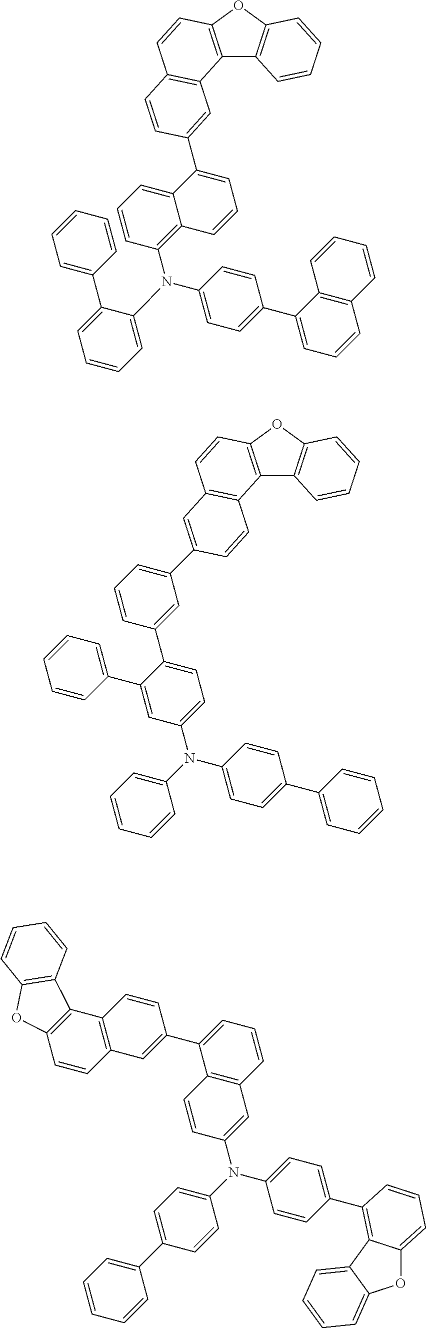 [SMILES: c1ccc(-c2ccccc2N(c2ccc(-c3cccc4ccccc34)cc2)c2cccc3c(-c4ccc5ccc6oc7ccccc7c6c5c4)cccc23)cc1 ; c1ccc(-c2ccc(N(c3ccccc3)c3ccc(-c4cccc(-c5ccc6c(ccc7oc8ccccc8c76)c5)c4)c(-c4ccccc4)c3)cc2)cc1 ; c1ccc(-c2ccc(N(c3ccc(-c4cccc5oc6ccccc6c45)cc3)c3ccc4c(-c5ccc6c(ccc7oc8ccccc8c76)c5)cccc4c3)cc2)cc1]

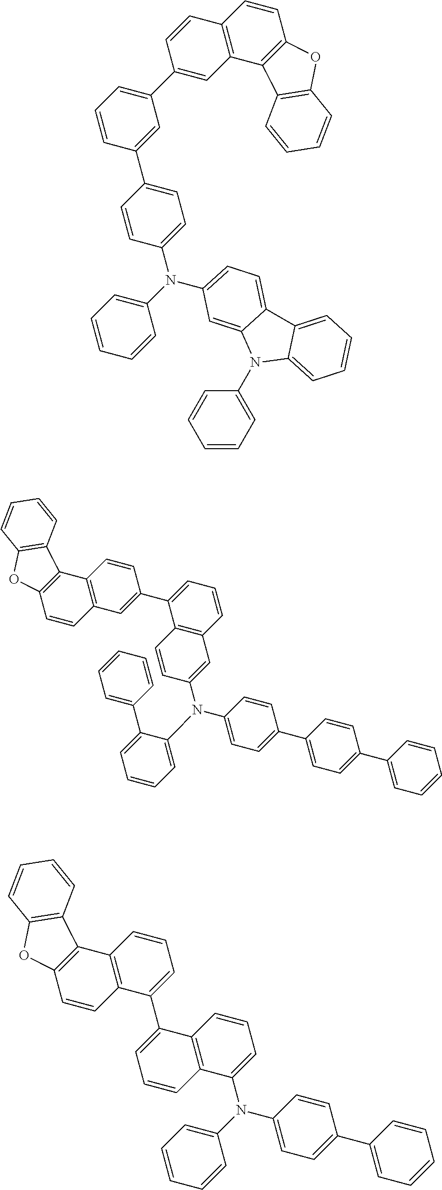 [SMILES: c1ccc(N(c2ccc(-c3cccc(-c4ccc5ccc6oc7ccccc7c6c5c4)c3)cc2)c2ccc3c4ccccc4n(-c4ccccc4)c3c2)cc1 ; c1ccc(-c2ccc(-c3ccc(N(c4ccc5c(-c6ccc7c(ccc8oc9ccccc9c87)c6)cccc5c4)c4ccccc4-c4ccccc4)cc3)cc2)cc1 ; c1ccc(-c2ccc(N(c3ccccc3)c3cccc4c(-c5cccc6c5ccc5oc7ccccc7c56)cccc34)cc2)cc1]

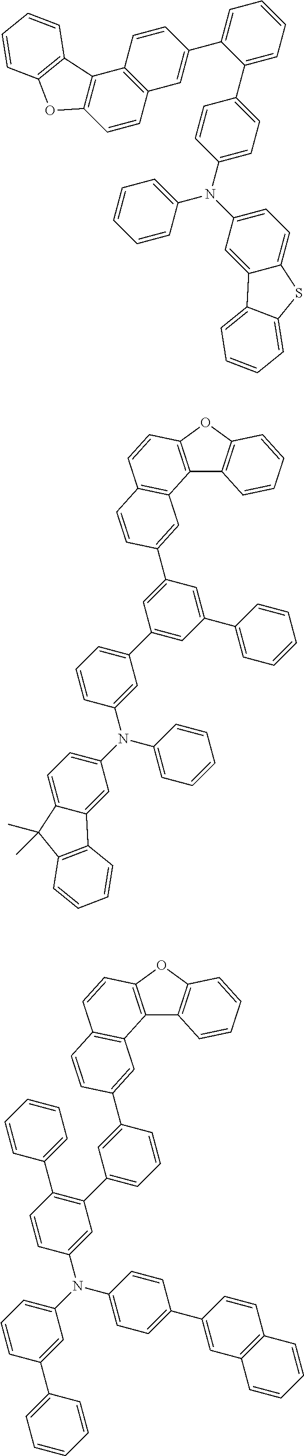 [SMILES: c1ccc(N(c2ccc(-c3ccccc3-c3ccc4c(ccc5oc6ccccc6c54)c3)cc2)c2ccc3sc4ccccc4c3c2)cc1 ; CC1(C)c2ccccc2-c2cc(N(c3ccccc3)c3cccc(-c4cc(-c5ccccc5)cc(-c5ccc6ccc7oc8ccccc8c7c6c5)c4)c3)ccc21 ; c1ccc(-c2cccc(N(c3ccc(-c4ccc5ccccc5c4)cc3)c3ccc(-c4ccccc4)c(-c4cccc(-c5ccc6ccc7oc8ccccc8c7c6c5)c4)c3)c2)cc1]

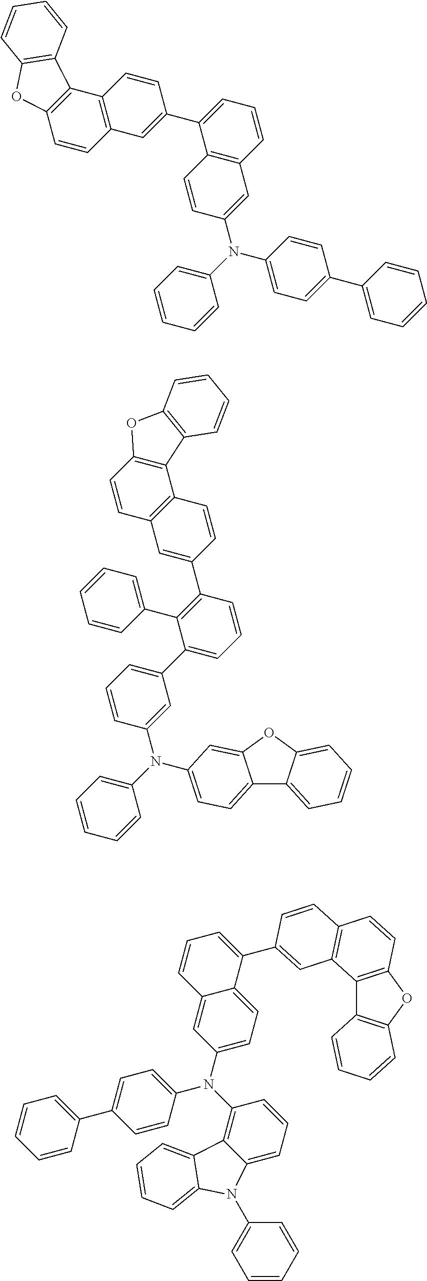

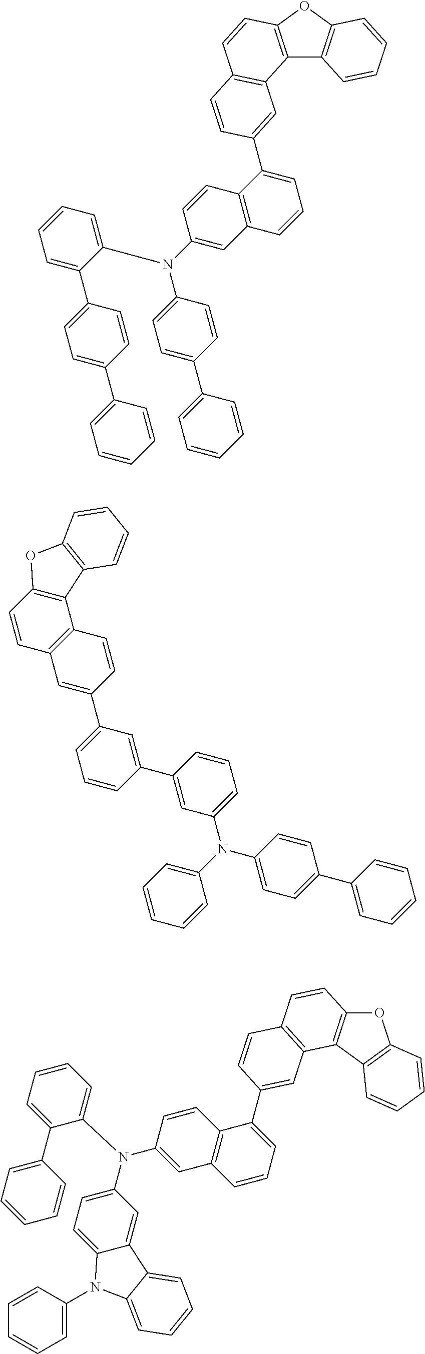 [SMILES: c1ccc(-c2ccc(-c3ccccc3N(c3ccc(-c4ccccc4)cc3)c3ccc4c(-c5ccc6ccc7oc8ccccc8c7c6c5)cccc4c3)cc2)cc1 ; c1ccc(-c2ccc(N(c3ccccc3)c3cccc(-c4cccc(-c5ccc6c(ccc7oc8ccccc8c76)c5)c4)c3)cc2)cc1 ; c1ccc(-c2ccccc2N(c2ccc3c(-c4ccc5ccc6oc7ccccc7c6c5c4)cccc3c2)c2ccc3c(c2)c2ccccc2n3-c2ccccc2)cc1]

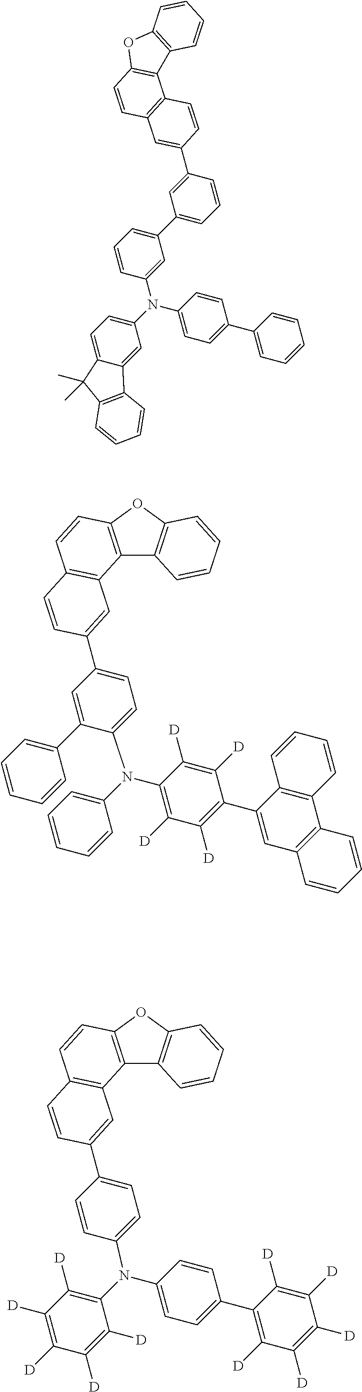 [SMILES: CC1(C)c2ccccc2-c2cc(N(c3ccc(-c4ccccc4)cc3)c3cccc(-c4cccc(-c5ccc6c(ccc7oc8ccccc8c76)c5)c4)c3)ccc21 ; [2H]c1c([2H])c(N(c2ccccc2)c2ccc(-c3ccc4ccc5oc6ccccc6c5c4c3)cc2-c2ccccc2)c([2H])c([2H])c1-c1cc2ccccc2c2ccccc12 ; [2H]c1c([2H])c([2H])c(-c2ccc(N(c3ccc(-c4ccc5ccc6oc7ccccc7c6c5c4)cc3)c3c([2H])c([2H])c([2H])c([2H])c3[2H])cc2)c([2H])c1[2H]]

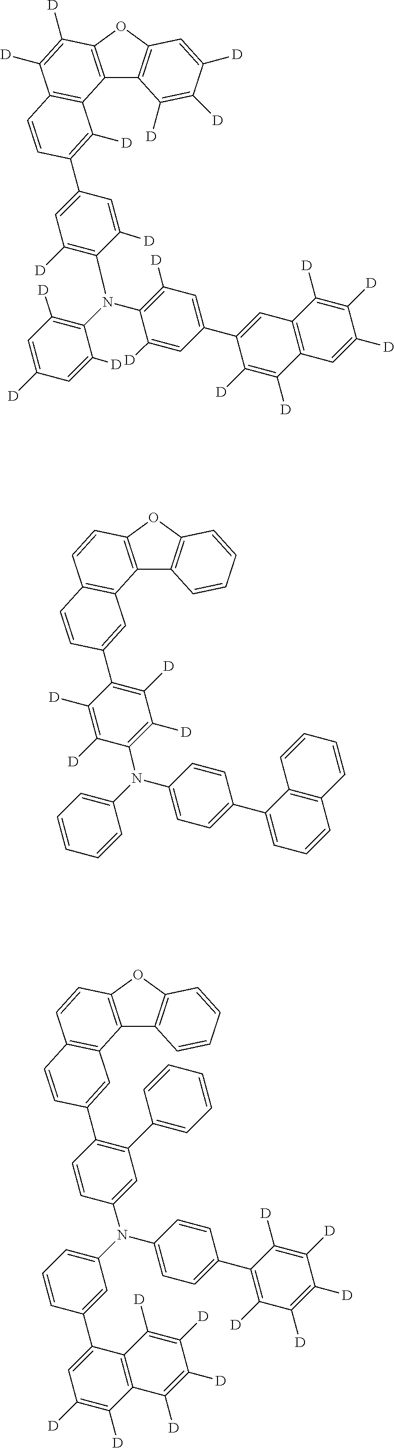 [SMILES: [2H]c1cc([2H])c(N(c2c([2H])cc(-c3cc4c([2H])c([2H])c([2H])cc4c([2H])c3[2H])cc2[2H])c2c([2H])cc(-c3ccc4c([2H])c([2H])c5oc6cc([2H])c([2H])c([2H])c6c5c4c3[2H])cc2[2H])c([2H])c1 ; [2H]c1c([2H])c(N(c2ccccc2)c2ccc(-c3cccc4ccccc34)cc2)c([2H])c([2H])c1-c1ccc2ccc3oc4ccccc4c3c2c1 ; [2H]c1cc(-c2cccc(N(c3ccc(-c4c([2H])c([2H])c([2H])c([2H])c4[2H])cc3)c3ccc(-c4ccc5ccc6oc7ccccc7c6c5c4)c(-c4ccccc4)c3)c2)c2c([2H])c([2H])c([2H])c([2H])c2c1[2H]]

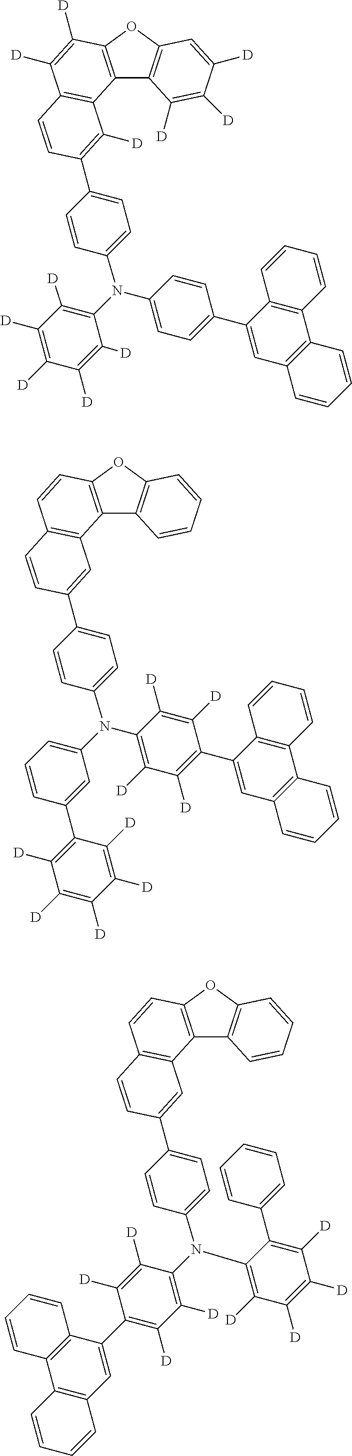 [SMILES: [2H]c1cc2oc3c([2H])c([2H])c4ccc(-c5ccc(N(c6ccc(-c7cc8ccccc8c8ccccc78)cc6)c6c([2H])c([2H])c([2H])c([2H])c6[2H])cc5)c([2H])c4c3c2c([2H])c1[2H] ; [2H]c1c([2H])c([2H])c(-c2cccc(N(c3ccc(-c4ccc5ccc6oc7ccccc7c6c5c4)cc3)c3c([2H])c([2H])c(-c4cc5ccccc5c5ccccc45)c([2H])c3[2H])c2)c([2H])c1[2H] ; [2H]c1c([2H])c([2H])c(N(c2ccc(-c3ccc4ccc5oc6ccccc6c5c4c3)cc2)c2c([2H])c([2H])c(-c3cc4ccccc4c4ccccc34)c([2H])c2[2H])c(-c2ccccc2)c1[2H]]

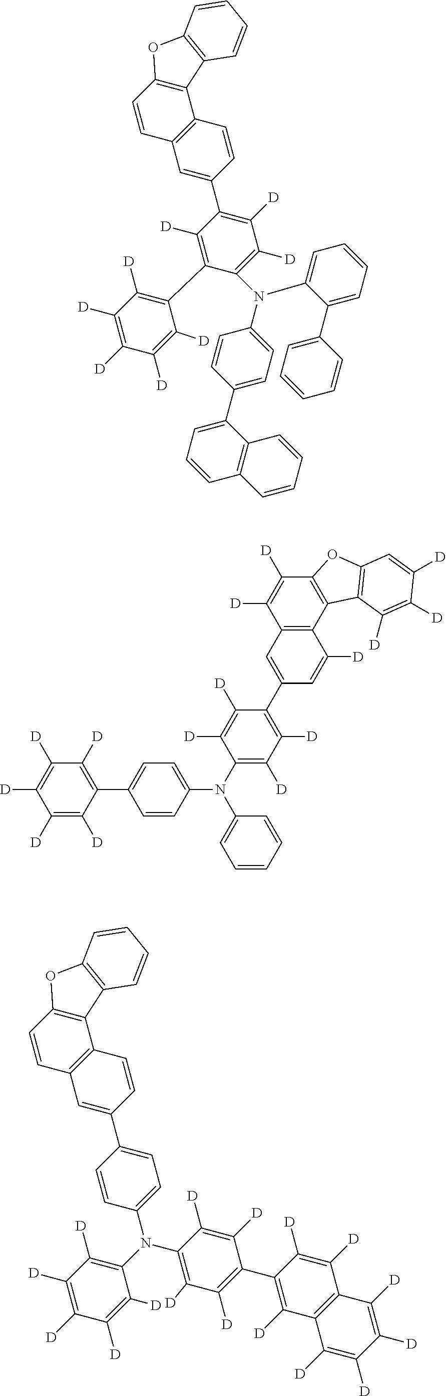 [SMILES: [2H]c1c([2H])c([2H])c(-c2c([2H])c(-c3ccc4c(ccc5oc6ccccc6c54)c3)c([2H])c([2H])c2N(c2ccc(-c3cccc4ccccc34)cc2)c2ccccc2-c2ccccc2)c([2H])c1[2H] ; [2H]c1cc2oc3c([2H])c([2H])c4cc(-c5c([2H])c([2H])c(N(c6ccccc6)c6ccc(-c7c([2H])c([2H])c([2H])c([2H])c7[2H])cc6)c([2H])c5[2H])cc([2H])c4c3c2c([2H])c1[2H] ; [2H]c1c([2H])c([2H])c(N(c2ccc(-c3ccc4c(ccc5oc6ccccc6c54)c3)cc2)c2c([2H])c([2H])c(-c3c([2H])c([2H])c4c([2H])c([2H])c([2H])c([2H])c4c3[2H])c([2H])c2[2H])c([2H])c1[2H]]

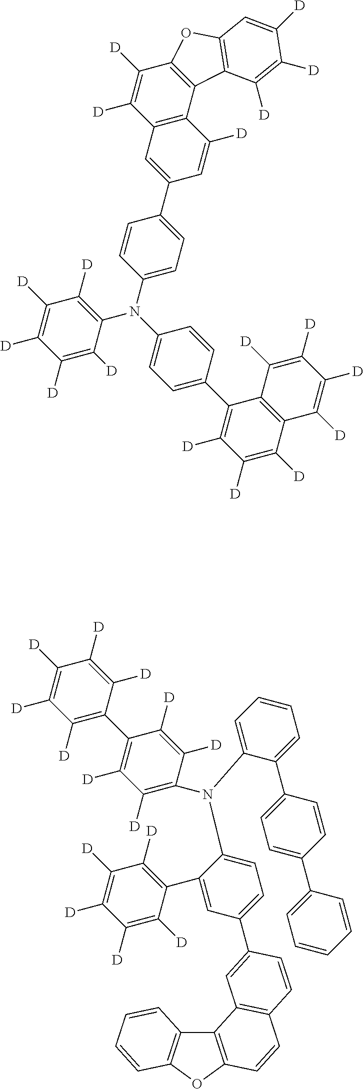 [SMILES: [2H]c1cc2oc3c([2H])c([2H])c4cc(-c5ccc(N(c6ccc(-c7c([2H])c([2H])c([2H])c8c([2H])c([2H])c([2H])c([2H])c78)cc6)c6c([2H])c([2H])c([2H])c([2H])c6[2H])cc5)cc([2H])c4c3c2c([2H])c1[2H] ; [2H]c1c([2H])c([2H])c(-c2cc(-c3ccc4ccc5oc6ccccc6c5c4c3)ccc2N(c2ccccc2-c2ccc(-c3ccccc3)cc2)c2c([2H])c([2H])c(-c3c([2H])c([2H])c([2H])c([2H])c3[2H])c([2H])c2[2H])c([2H])c1[2H]]

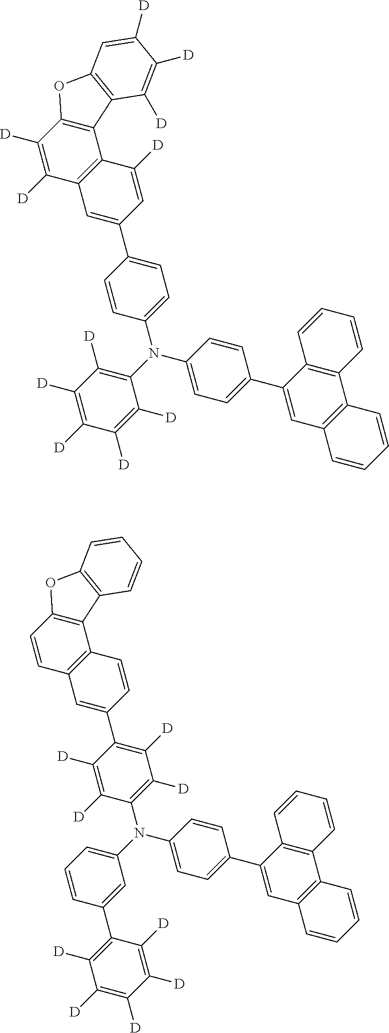 [SMILES: [2H]c1cc2oc3c([2H])c([2H])c4cc(-c5ccc(N(c6ccc(-c7cc8ccccc8c8ccccc78)cc6)c6c([2H])c([2H])c([2H])c([2H])c6[2H])cc5)cc([2H])c4c3c2c([2H])c1[2H] ; [2H]c1c([2H])c([2H])c(-c2cccc(N(c3ccc(-c4cc5ccccc5c5ccccc45)cc3)c3c([2H])c([2H])c(-c4ccc5c(ccc6oc7ccccc7c65)c4)c([2H])c3[2H])c2)c([2H])c1[2H]]

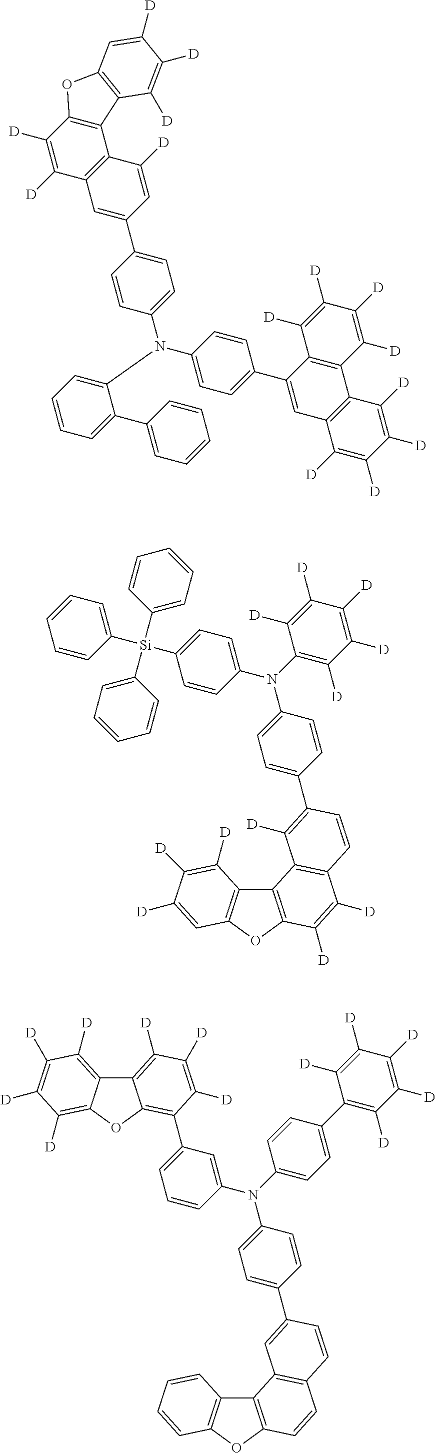 [SMILES: [2H]c1cc2oc3c([2H])c([2H])c4cc(-c5ccc(N(c6ccc(-c7cc8c([2H])c([2H])c([2H])c([2H])c8c8c([2H])c([2H])c([2H])c([2H])c78)cc6)c6ccccc6-c6ccccc6)cc5)cc([2H])c4c3c2c([2H])c1[2H] ; [2H]c1cc2oc3c([2H])c([2H])c4ccc(-c5ccc(N(c6ccc([Si](c7ccccc7)(c7ccccc7)c7ccccc7)cc6)c6c([2H])c([2H])c([2H])c([2H])c6[2H])cc5)c([2H])c4c3c2c([2H])c1[2H] ; [2H]c1c([2H])c([2H])c(-c2ccc(N(c3ccc(-c4ccc5ccc6oc7ccccc7c6c5c4)cc3)c3cccc(-c4c([2H])c([2H])c([2H])c5c4oc4c([2H])c([2H])c([2H])c([2H])c45)c3)cc2)c([2H])c1[2H]]

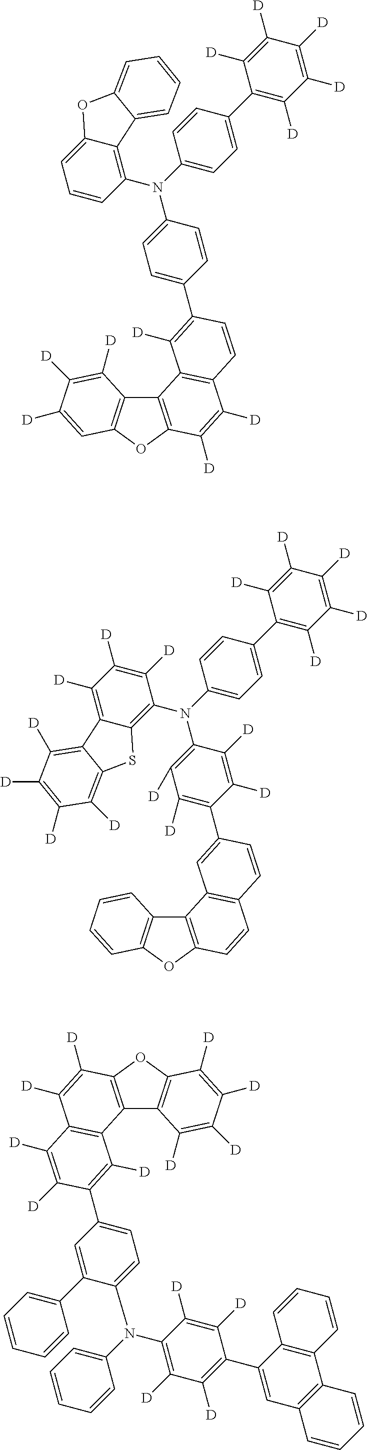 [SMILES: [2H]c1cc2oc3c([2H])c([2H])c4ccc(-c5ccc(N(c6ccc(-c7c([2H])c([2H])c([2H])c([2H])c7[2H])cc6)c6cccc7oc8ccccc8c67)cc5)c([2H])c4c3c2c([2H])c1[2H] ; [2H]c1c([2H])c([2H])c(-c2ccc(N(c3c([2H])c([2H])c(-c4ccc5ccc6oc7ccccc7c6c5c4)c([2H])c3[2H])c3c([2H])c([2H])c([2H])c4c3sc3c([2H])c([2H])c([2H])c([2H])c34)cc2)c([2H])c1[2H] ; [2H]c1c([2H])c(N(c2ccccc2)c2ccc(-c3c([2H])c([2H])c4c([2H])c([2H])c5oc6c([2H])c([2H])c([2H])c([2H])c6c5c4c3[2H])cc2-c2ccccc2)c([2H])c([2H])c1-c1cc2ccccc2c2ccccc12]

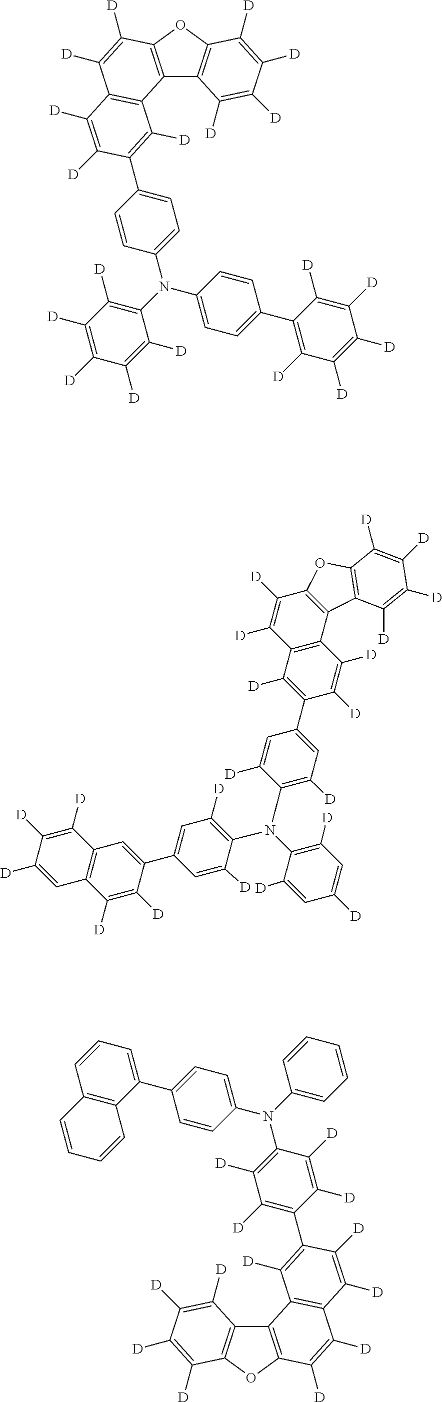

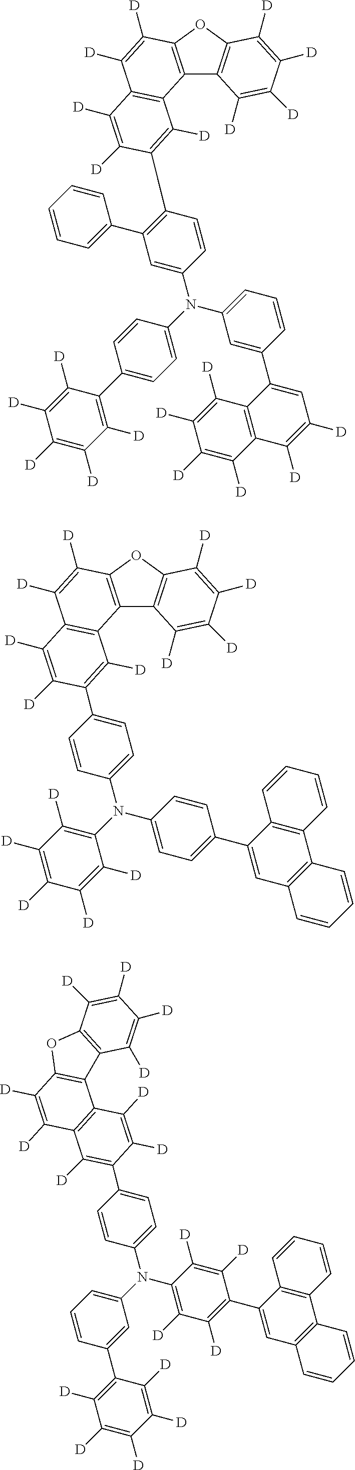

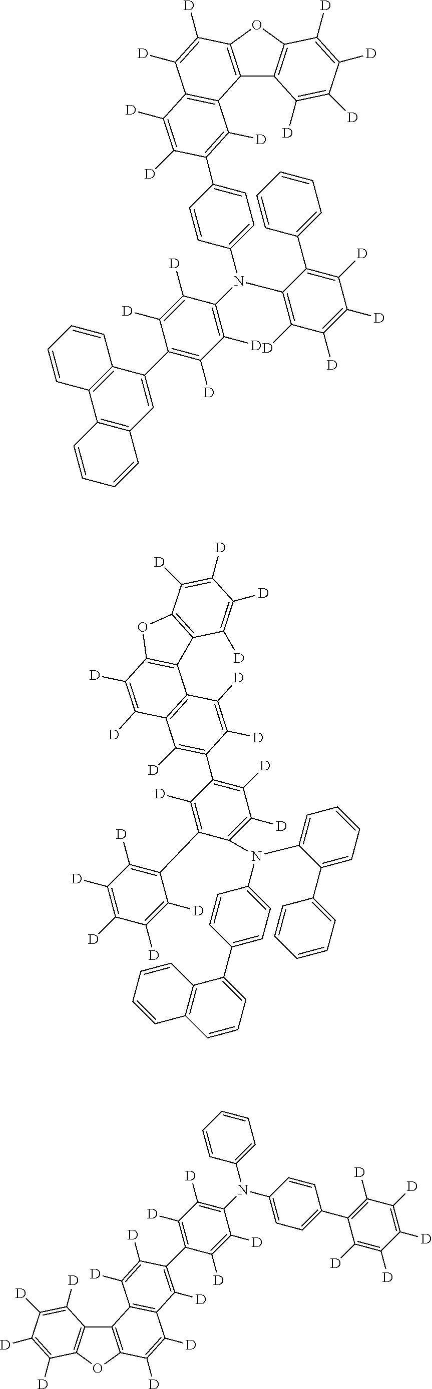

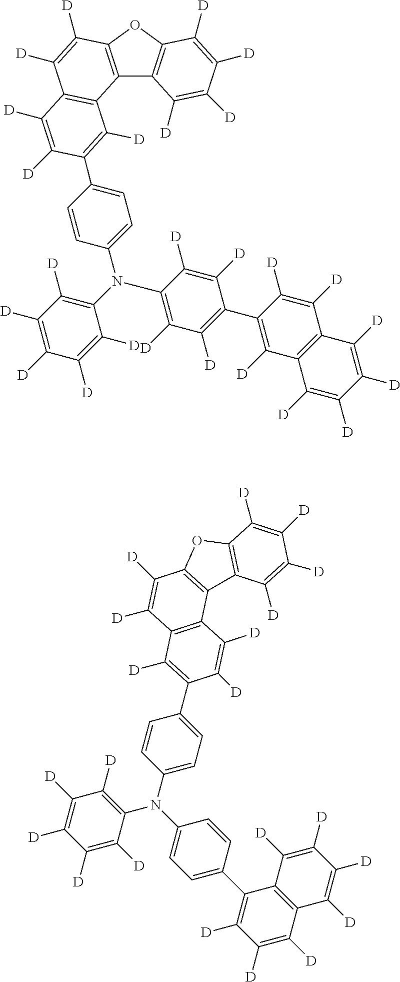 [SMILES: [2H]c1c([2H])c([2H])c(N(c2ccc(-c3c([2H])c([2H])c4c([2H])c([2H])c5oc6c([2H])c([2H])c([2H])c([2H])c6c5c4c3[2H])cc2)c2c([2H])c([2H])c(-c3c([2H])c([2H])c4c([2H])c([2H])c([2H])c([2H])c4c3[2H])c([2H])c2[2H])c([2H])c1[2H] ; [2H]c1c([2H])c([2H])c(N(c2ccc(-c3c([2H])c([2H])c4c(c3[2H])c([2H])c([2H])c3oc5c([2H])c([2H])c([2H])c([2H])c5c34)cc2)c2ccc(-c3c([2H])c([2H])c([2H])c4c([2H])c([2H])c([2H])c([2H])c34)cc2)c([2H])c1[2H]]

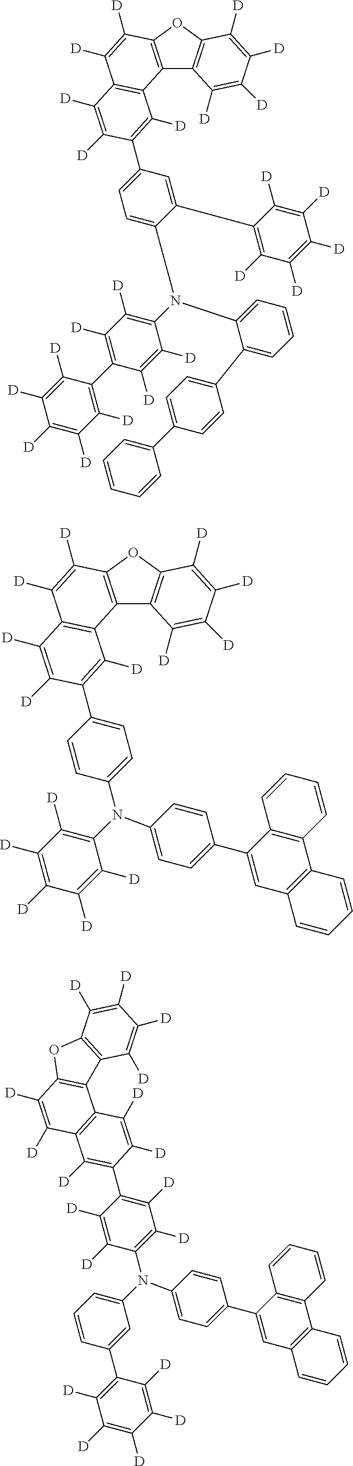

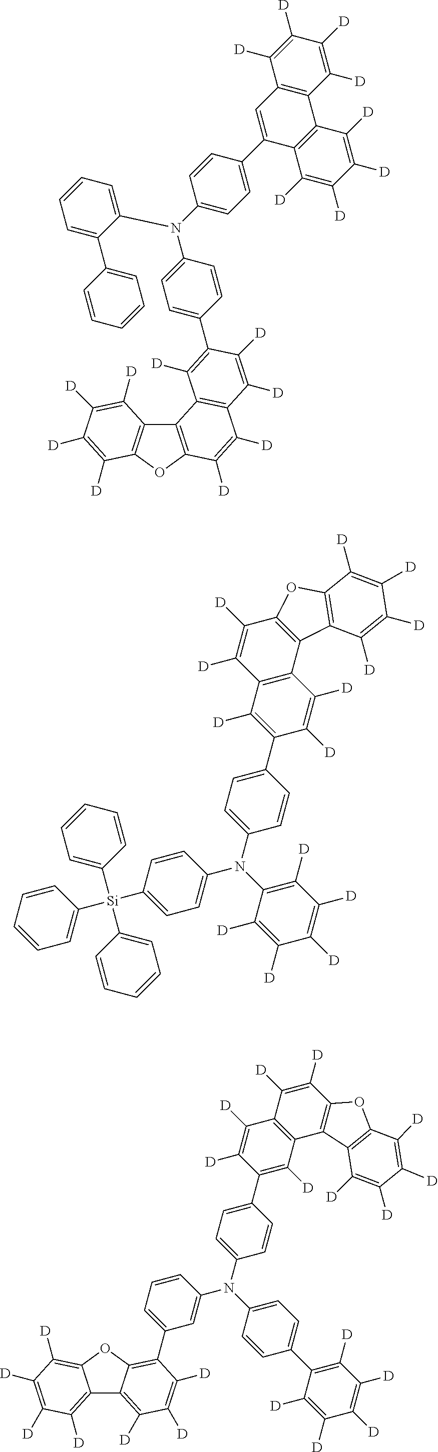

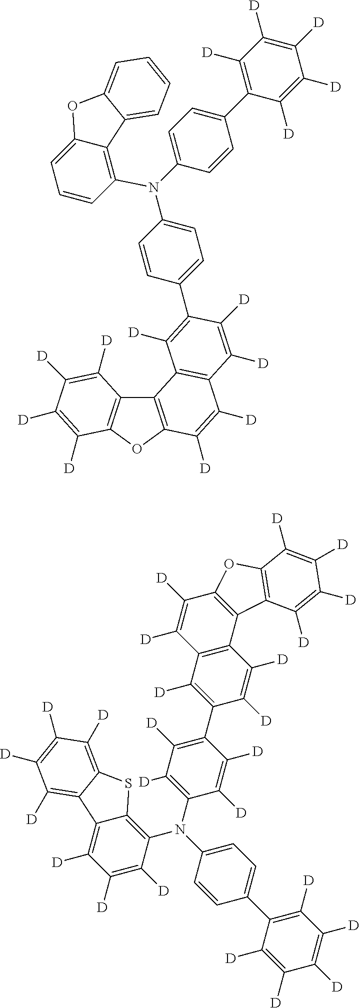 [SMILES: [2H]c1c([2H])c([2H])c(-c2ccc(N(c3ccc(-c4c([2H])c([2H])c5c([2H])c([2H])c6oc7c([2H])c([2H])c([2H])c([2H])c7c6c5c4[2H])cc3)c3cccc4oc5ccccc5c34)cc2)c([2H])c1[2H] ; [2H]c1c([2H])c([2H])c(-c2ccc(N(c3c([2H])c([2H])c(-c4c([2H])c([2H])c5c(c4[2H])c([2H])c([2H])c4oc6c([2H])c([2H])c([2H])c([2H])c6c45)c([2H])c3[2H])c3c([2H])c([2H])c([2H])c4c3sc3c([2H])c([2H])c([2H])c([2H])c34)cc2)c([2H])c1[2H]]

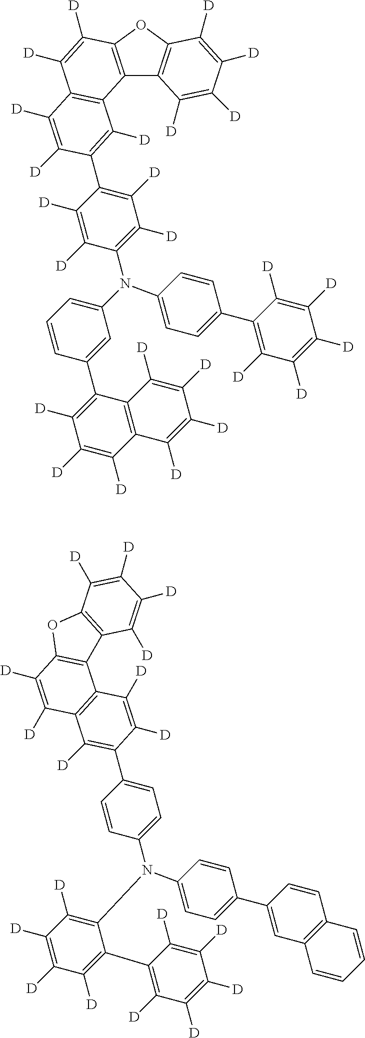 [SMILES: [2H]c1c([2H])c([2H])c(-c2ccc(N(c3cccc(-c4c([2H])c([2H])c([2H])c5c([2H])c([2H])c([2H])c([2H])c45)c3)c3c([2H])c([2H])c(-c4c([2H])c([2H])c5c([2H])c([2H])c6oc7c([2H])c([2H])c([2H])c([2H])c7c6c5c4[2H])c([2H])c3[2H])cc2)c([2H])c1[2H] ; [2H]c1c([2H])c([2H])c(-c2c([2H])c([2H])c([2H])c([2H])c2N(c2ccc(-c3ccc4ccccc4c3)cc2)c2ccc(-c3c([2H])c([2H])c4c(c3[2H])c([2H])c([2H])c3oc5c([2H])c([2H])c([2H])c([2H])c5c34)cc2)c([2H])c1[2H]]

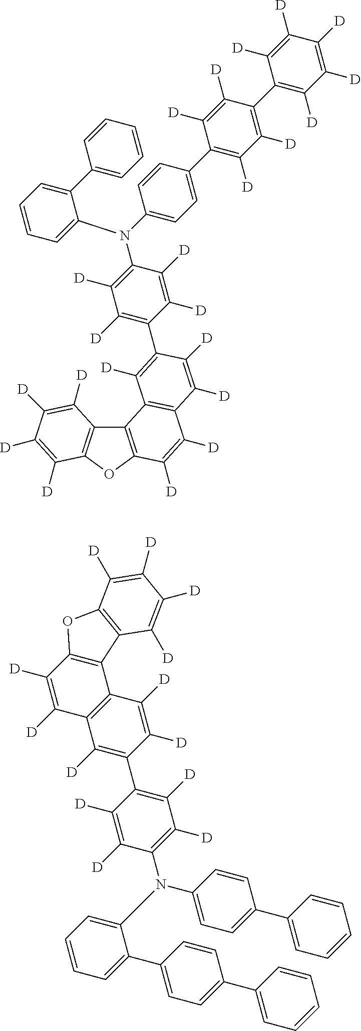 [SMILES: [2H]c1c([2H])c([2H])c(-c2c([2H])c([2H])c(-c3ccc(N(c4ccccc4-c4ccccc4)c4c([2H])c([2H])c(-c5c([2H])c([2H])c6c([2H])c([2H])c7oc8c([2H])c([2H])c([2H])c([2H])c8c7c6c5[2H])c([2H])c4[2H])cc3)c([2H])c2[2H])c([2H])c1[2H] ; [2H]c1c([2H])c(N(c2ccc(-c3ccccc3)cc2)c2ccccc2-c2ccc(-c3ccccc3)cc2)c([2H])c([2H])c1-c1c([2H])c([2H])c2c(c1[2H])c([2H])c([2H])c1oc3c([2H])c([2H])c([2H])c([2H])c3c12]

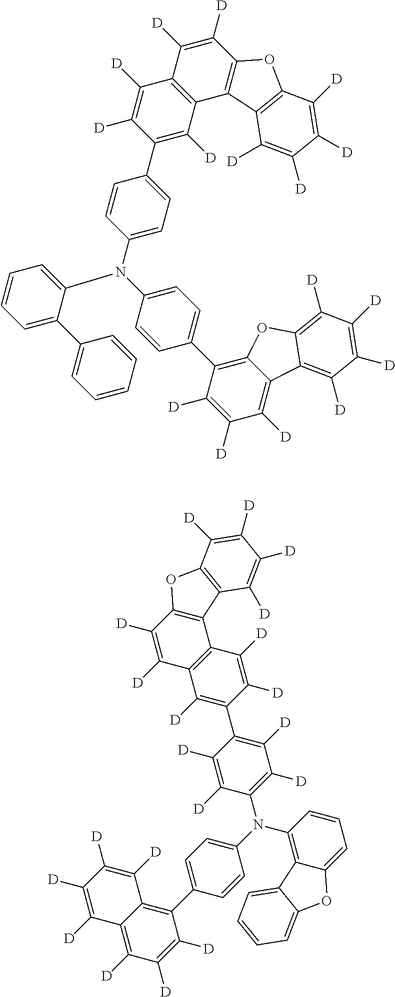 [SMILES: [2H]c1c([2H])c([2H])c2c(oc3c(-c4ccc(N(c5ccc(-c6c([2H])c([2H])c7c([2H])c([2H])c8oc9c([2H])c([2H])c([2H])c([2H])c9c8c7c6[2H])cc5)c5ccccc5-c5ccccc5)cc4)c([2H])c([2H])c([2H])c32)c1[2H] ; [2H]c1c([2H])c(N(c2ccc(-c3c([2H])c([2H])c([2H])c4c([2H])c([2H])c([2H])c([2H])c34)cc2)c2cccc3oc4ccccc4c23)c([2H])c([2H])c1-c1c([2H])c([2H])c2c(c1[2H])c([2H])c([2H])c1oc3c([2H])c([2H])c([2H])c([2H])c3c12]

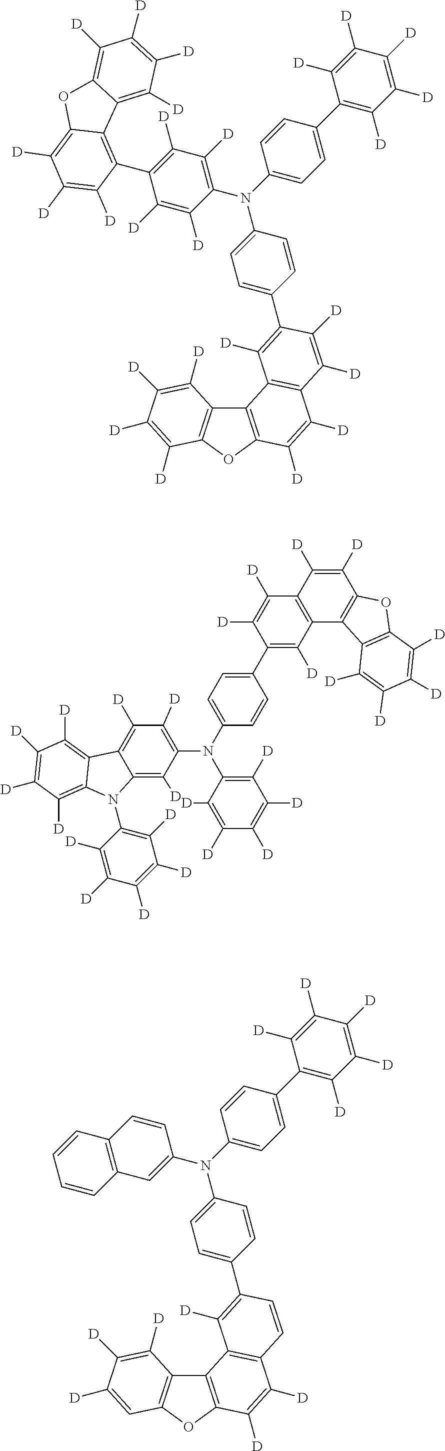

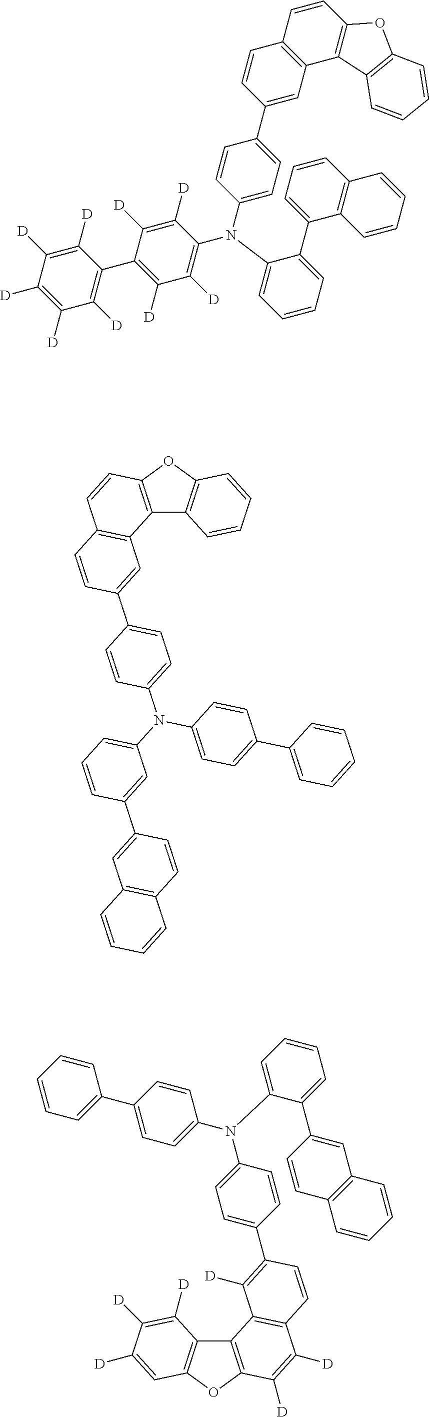 [SMILES: [2H]c1c([2H])c([2H])c(-c2c([2H])c([2H])c(N(c3ccc(-c4ccc5ccc6oc7ccccc7c6c5c4)cc3)c3ccccc3-c3cccc4ccccc34)c([2H])c2[2H])c([2H])c1[2H] ; c1ccc(-c2ccc(N(c3ccc(-c4ccc5ccc6oc7ccccc7c6c5c4)cc3)c3cccc(-c4ccc5ccccc5c4)c3)cc2)cc1 ; [2H]c1cc2oc3c([2H])c([2H])c4ccc(-c5ccc(N(c6ccc(-c7ccccc7)cc6)c6ccccc6-c6ccc7ccccc7c6)cc5)c([2H])c4c3c2c([2H])c1[2H]]

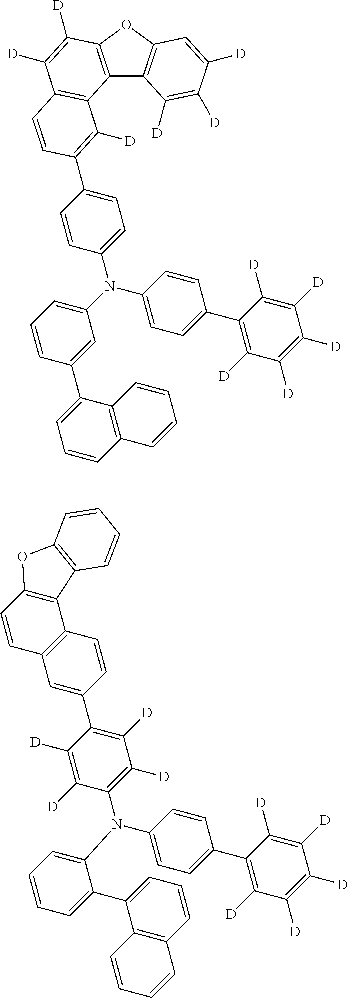

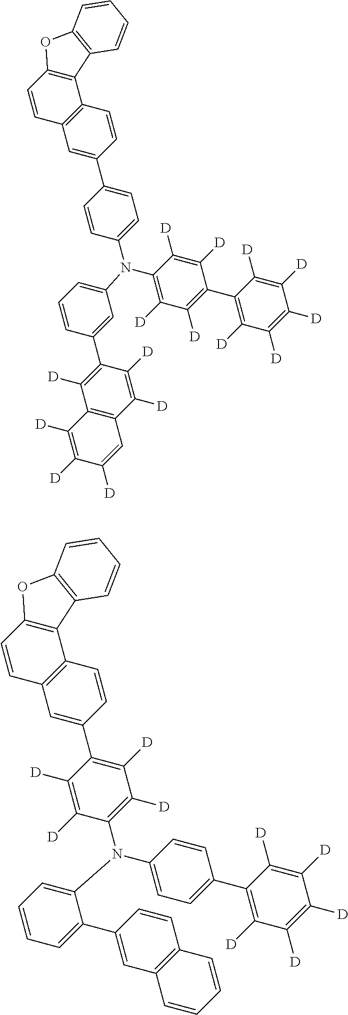 [SMILES: [2H]c1cc2c([2H])c([2H])c(-c3cccc(N(c4ccc(-c5ccc6c(ccc7oc8ccccc8c76)c5)cc4)c4c([2H])c([2H])c(-c5c([2H])c([2H])c([2H])c([2H])c5[2H])c([2H])c4[2H])c3)c([2H])c2c([2H])c1[2H] ; [2H]c1c([2H])c([2H])c(-c2ccc(N(c3ccccc3-c3ccc4ccccc4c3)c3c([2H])c([2H])c(-c4ccc5c(ccc6oc7ccccc7c65)c4)c([2H])c3[2H])cc2)c([2H])c1[2H]]

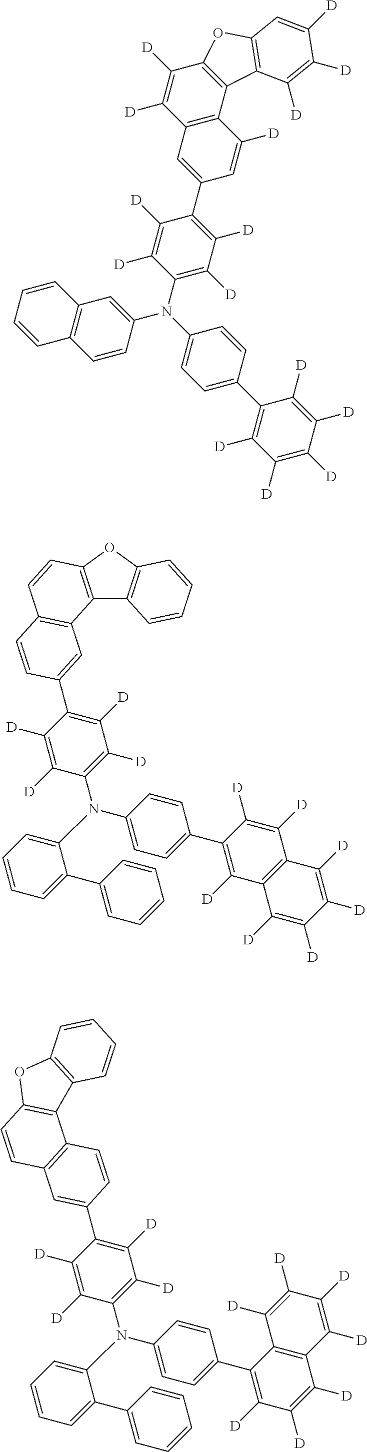 [SMILES: [2H]c1cc2oc3c([2H])c([2H])c4cc(-c5c([2H])c([2H])c(N(c6ccc(-c7c([2H])c([2H])c([2H])c([2H])c7[2H])cc6)c6ccc7ccccc7c6)c([2H])c5[2H])cc([2H])c4c3c2c([2H])c1[2H] ; [2H]c1c([2H])c(N(c2ccc(-c3c([2H])c([2H])c4c([2H])c([2H])c([2H])c([2H])c4c3[2H])cc2)c2ccccc2-c2ccccc2)c([2H])c([2H])c1-c1ccc2ccc3oc4ccccc4c3c2c1 ; [2H]c1c([2H])c(N(c2ccc(-c3c([2H])c([2H])c([2H])c4c([2H])c([2H])c([2H])c([2H])c34)cc2)c2ccccc2-c2ccccc2)c([2H])c([2H])c1-c1ccc2c(ccc3oc4ccccc4c32)c1]

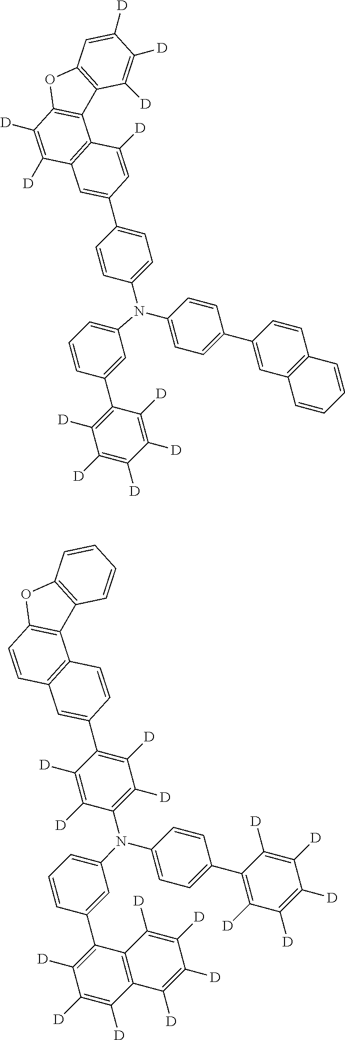 [SMILES: [2H]c1cc2oc3c([2H])c([2H])c4cc(-c5ccc(N(c6ccc(-c7ccc8ccccc8c7)cc6)c6cccc(-c7c([2H])c([2H])c([2H])c([2H])c7[2H])c6)cc5)cc([2H])c4c3c2c([2H])c1[2H] ; [2H]c1c([2H])c([2H])c(-c2ccc(N(c3cccc(-c4c([2H])c([2H])c([2H])c5c([2H])c([2H])c([2H])c([2H])c45)c3)c3c([2H])c([2H])c(-c4ccc5c(ccc6oc7ccccc7c65)c4)c([2H])c3[2H])cc2)c([2H])c1[2H]]

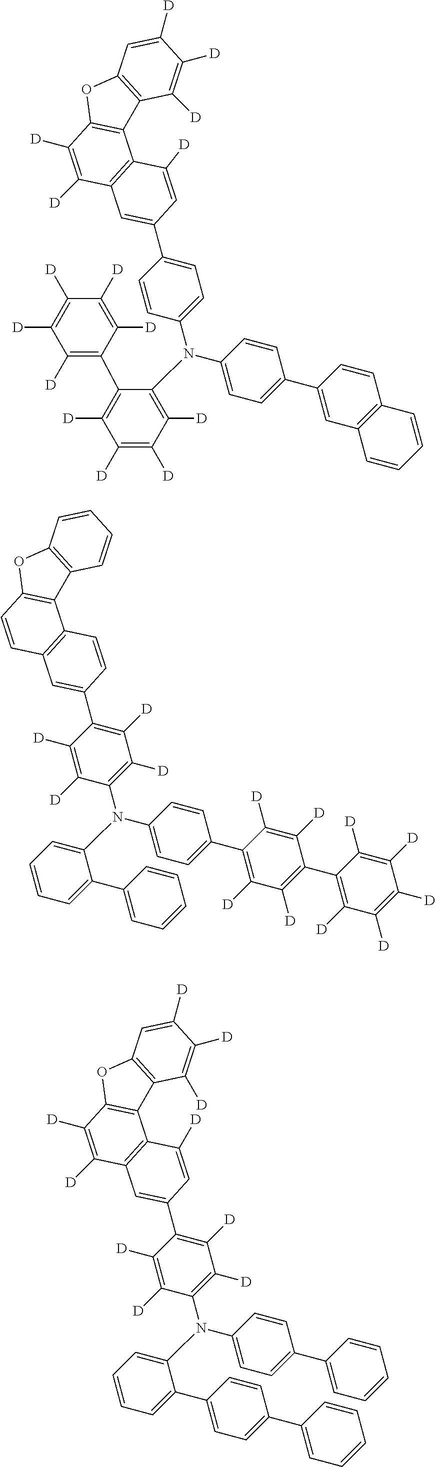

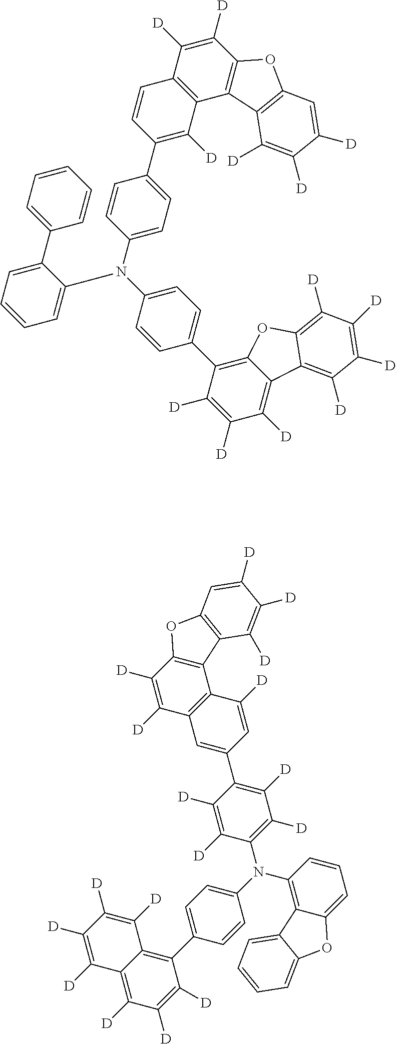 [SMILES: [2H]c1cc2oc3c([2H])c([2H])c4ccc(-c5ccc(N(c6ccc(-c7c([2H])c([2H])c([2H])c8c7oc7c([2H])c([2H])c([2H])c([2H])c78)cc6)c6ccccc6-c6ccccc6)cc5)c([2H])c4c3c2c([2H])c1[2H] ; [2H]c1cc2oc3c([2H])c([2H])c4cc(-c5c([2H])c([2H])c(N(c6ccc(-c7c([2H])c([2H])c([2H])c8c([2H])c([2H])c([2H])c([2H])c78)cc6)c6cccc7oc8ccccc8c67)c([2H])c5[2H])cc([2H])c4c3c2c([2H])c1[2H]]

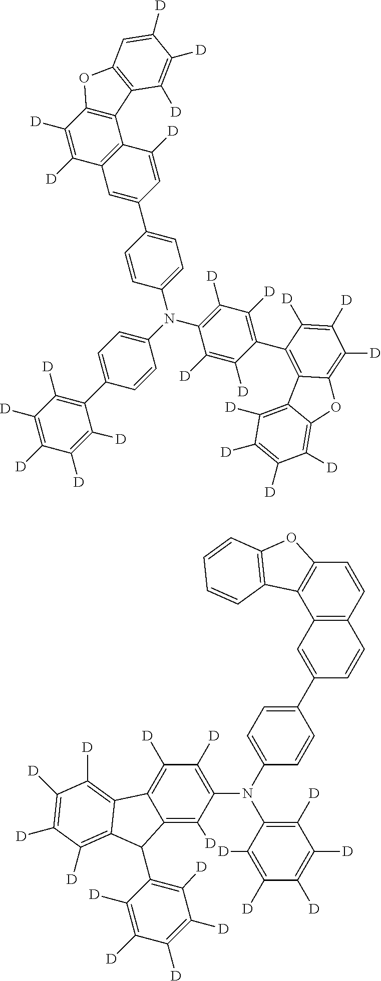 [SMILES: [2H]c1cc2oc3c([2H])c([2H])c4cc(-c5ccc(N(c6ccc(-c7c([2H])c([2H])c([2H])c([2H])c7[2H])cc6)c6c([2H])c([2H])c(-c7c([2H])c([2H])c([2H])c8oc9c([2H])c([2H])c([2H])c([2H])c9c78)c([2H])c6[2H])cc5)cc([2H])c4c3c2c([2H])c1[2H] ; [2H]c1c([2H])c([2H])c(C2c3c([2H])c([2H])c([2H])c([2H])c3-c3c([2H])c([2H])c(N(c4ccc(-c5ccc6ccc7oc8ccccc8c7c6c5)cc4)c4c([2H])c([2H])c([2H])c([2H])c4[2H])c([2H])c32)c([2H])c1[2H]]

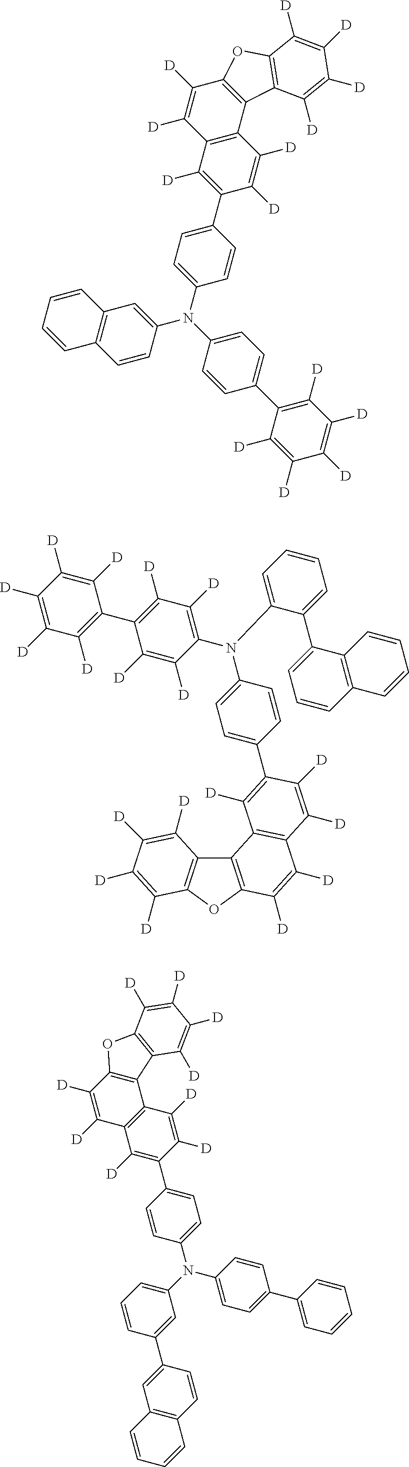 [SMILES: [2H]c1c([2H])c([2H])c(-c2ccc(N(c3ccc(-c4c([2H])c([2H])c5c(c4[2H])c([2H])c([2H])c4oc6c([2H])c([2H])c([2H])c([2H])c6c45)cc3)c3ccc4ccccc4c3)cc2)c([2H])c1[2H] ; [2H]c1c([2H])c([2H])c(-c2c([2H])c([2H])c(N(c3ccc(-c4c([2H])c([2H])c5c([2H])c([2H])c6oc7c([2H])c([2H])c([2H])c([2H])c7c6c5c4[2H])cc3)c3ccccc3-c3cccc4ccccc34)c([2H])c2[2H])c([2H])c1[2H] ; [2H]c1c([2H])c([2H])c2c(oc3c([2H])c([2H])c4c([2H])c(-c5ccc(N(c6ccc(-c7ccccc7)cc6)c6cccc(-c7ccc8ccccc8c7)c6)cc5)c([2H])c([2H])c4c32)c1[2H]]

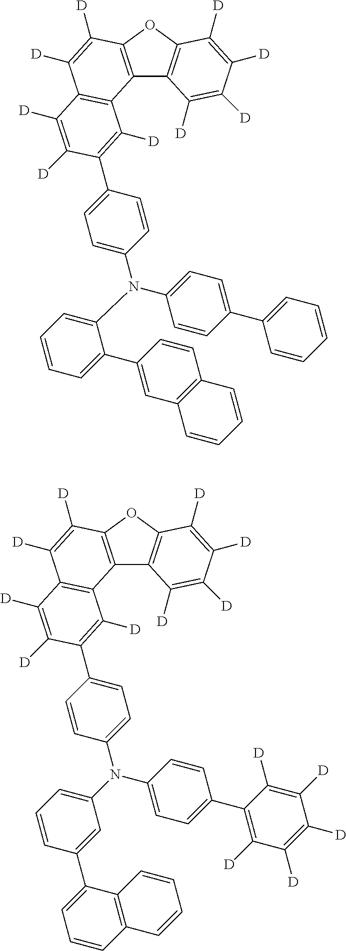 [SMILES: [2H]c1c([2H])c([2H])c2c(oc3c([2H])c([2H])c4c([2H])c([2H])c(-c5ccc(N(c6ccc(-c7ccccc7)cc6)c6ccccc6-c6ccc7ccccc7c6)cc5)c([2H])c4c32)c1[2H] ; [2H]c1c([2H])c([2H])c(-c2ccc(N(c3ccc(-c4c([2H])c([2H])c5c([2H])c([2H])c6oc7c([2H])c([2H])c([2H])c([2H])c7c6c5c4[2H])cc3)c3cccc(-c4cccc5ccccc45)c3)cc2)c([2H])c1[2H]]

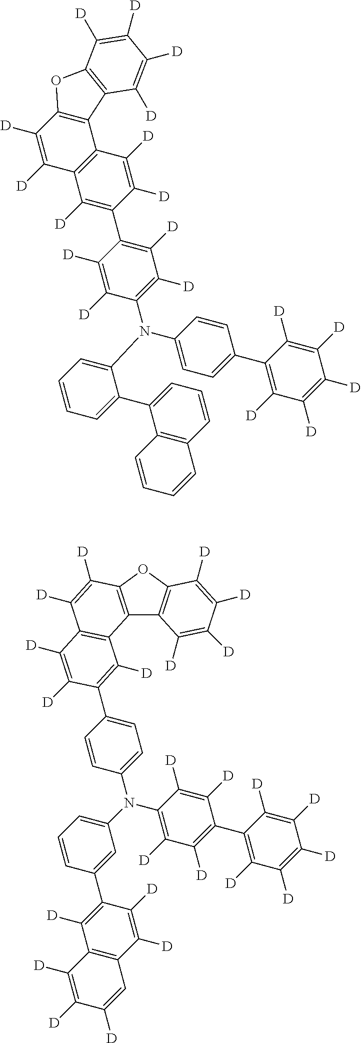 [SMILES: [2H]c1c([2H])c([2H])c(-c2ccc(N(c3ccccc3-c3cccc4ccccc34)c3c([2H])c([2H])c(-c4c([2H])c([2H])c5c(c4[2H])c([2H])c([2H])c4oc6c([2H])c([2H])c([2H])c([2H])c6c45)c([2H])c3[2H])cc2)c([2H])c1[2H] ; [2H]c1cc2c([2H])c([2H])c(-c3cccc(N(c4ccc(-c5c([2H])c([2H])c6c([2H])c([2H])c7oc8c([2H])c([2H])c([2H])c([2H])c8c7c6c5[2H])cc4)c4c([2H])c([2H])c(-c5c([2H])c([2H])c([2H])c([2H])c5[2H])c([2H])c4[2H])c3)c([2H])c2c([2H])c1[2H]]

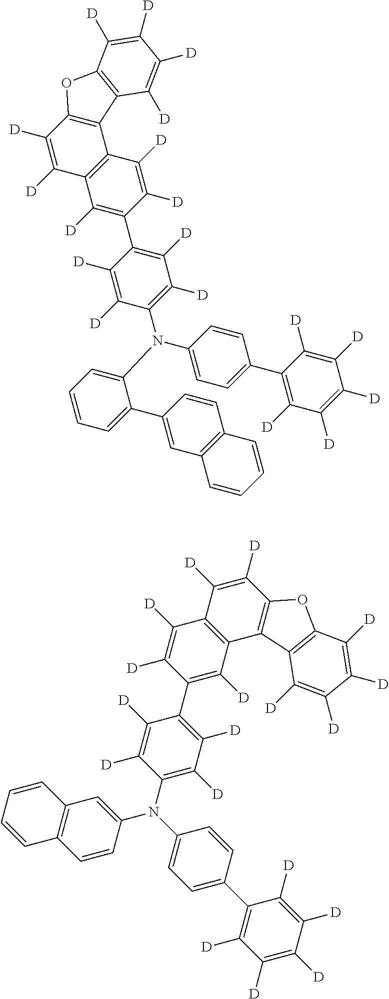 [SMILES: [2H]c1c([2H])c([2H])c(-c2ccc(N(c3ccccc3-c3ccc4ccccc4c3)c3c([2H])c([2H])c(-c4c([2H])c([2H])c5c(c4[2H])c([2H])c([2H])c4oc6c([2H])c([2H])c([2H])c([2H])c6c45)c([2H])c3[2H])cc2)c([2H])c1[2H] ; [2H]c1c([2H])c([2H])c(-c2ccc(N(c3ccc4ccccc4c3)c3c([2H])c([2H])c(-c4c([2H])c([2H])c5c([2H])c([2H])c6oc7c([2H])c([2H])c([2H])c([2H])c7c6c5c4[2H])c([2H])c3[2H])cc2)c([2H])c1[2H]]

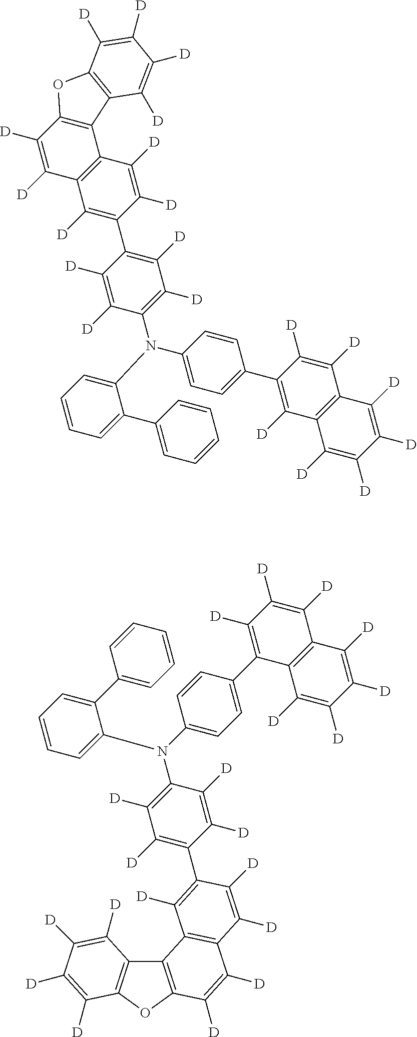 [SMILES: [2H]c1c([2H])c(N(c2ccc(-c3c([2H])c([2H])c4c([2H])c([2H])c([2H])c([2H])c4c3[2H])cc2)c2ccccc2-c2ccccc2)c([2H])c([2H])c1-c1c([2H])c([2H])c2c(c1[2H])c([2H])c([2H])c1oc3c([2H])c([2H])c([2H])c([2H])c3c12 ; [2H]c1c([2H])c(N(c2ccc(-c3c([2H])c([2H])c([2H])c4c([2H])c([2H])c([2H])c([2H])c34)cc2)c2ccccc2-c2ccccc2)c([2H])c([2H])c1-c1c([2H])c([2H])c2c([2H])c([2H])c3oc4c([2H])c([2H])c([2H])c([2H])c4c3c2c1[2H]]

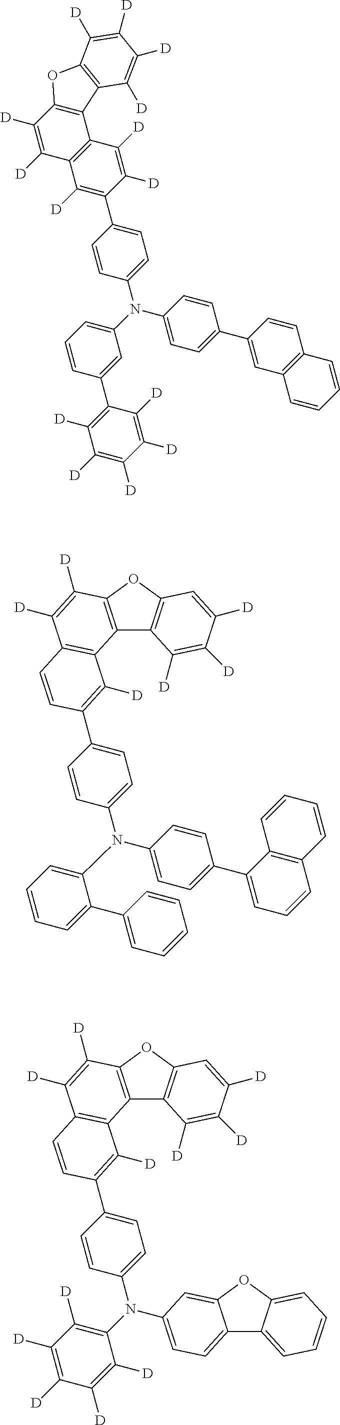 [SMILES: [2H]c1c([2H])c([2H])c(-c2cccc(N(c3ccc(-c4ccc5ccccc5c4)cc3)c3ccc(-c4c([2H])c([2H])c5c(c4[2H])c([2H])c([2H])c4oc6c([2H])c([2H])c([2H])c([2H])c6c45)cc3)c2)c([2H])c1[2H] ; [2H]c1cc2oc3c([2H])c([2H])c4ccc(-c5ccc(N(c6ccc(-c7cccc8ccccc78)cc6)c6ccccc6-c6ccccc6)cc5)c([2H])c4c3c2c([2H])c1[2H] ; [2H]c1cc2oc3c([2H])c([2H])c4ccc(-c5ccc(N(c6ccc7c(c6)oc6ccccc67)c6c([2H])c([2H])c([2H])c([2H])c6[2H])cc5)c([2H])c4c3c2c([2H])c1[2H]]

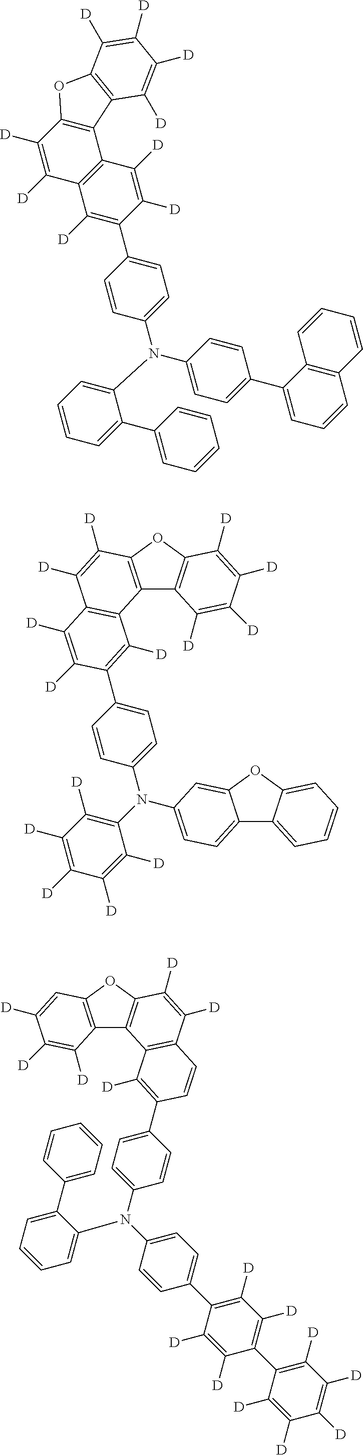 [SMILES: [2H]c1c([2H])c([2H])c2c(oc3c([2H])c([2H])c4c([2H])c(-c5ccc(N(c6ccc(-c7cccc8ccccc78)cc6)c6ccccc6-c6ccccc6)cc5)c([2H])c([2H])c4c32)c1[2H] ; [2H]c1c([2H])c([2H])c(N(c2ccc(-c3c([2H])c([2H])c4c([2H])c([2H])c5oc6c([2H])c([2H])c([2H])c([2H])c6c5c4c3[2H])cc2)c2ccc3c(c2)oc2ccccc23)c([2H])c1[2H] ; [2H]c1cc2oc3c([2H])c([2H])c4ccc(-c5ccc(N(c6ccc(-c7c([2H])c([2H])c(-c8c([2H])c([2H])c([2H])c([2H])c8[2H])c([2H])c7[2H])cc6)c6ccccc6-c6ccccc6)cc5)c([2H])c4c3c2c([2H])c1[2H]]

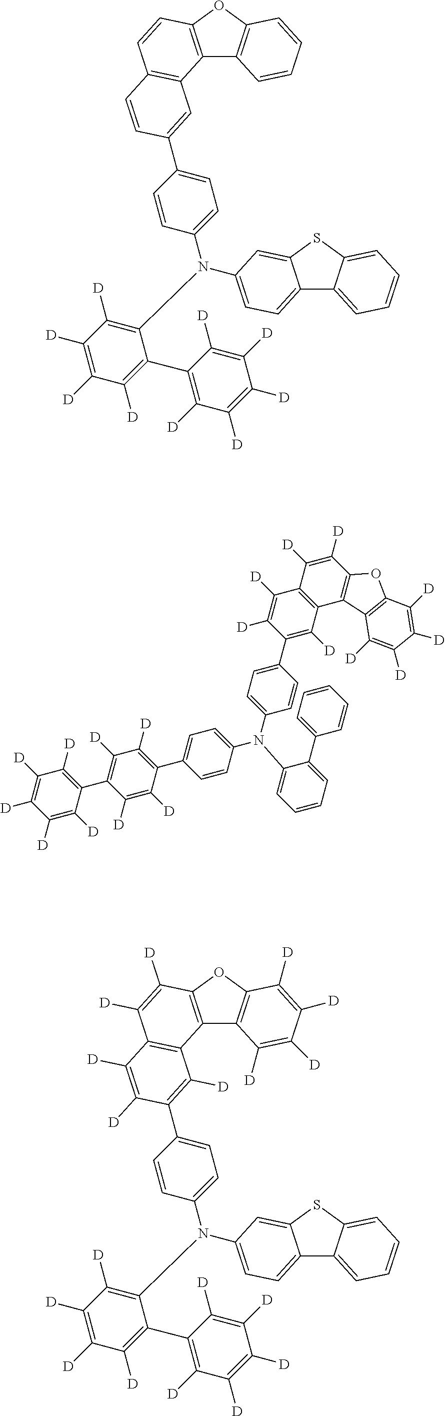

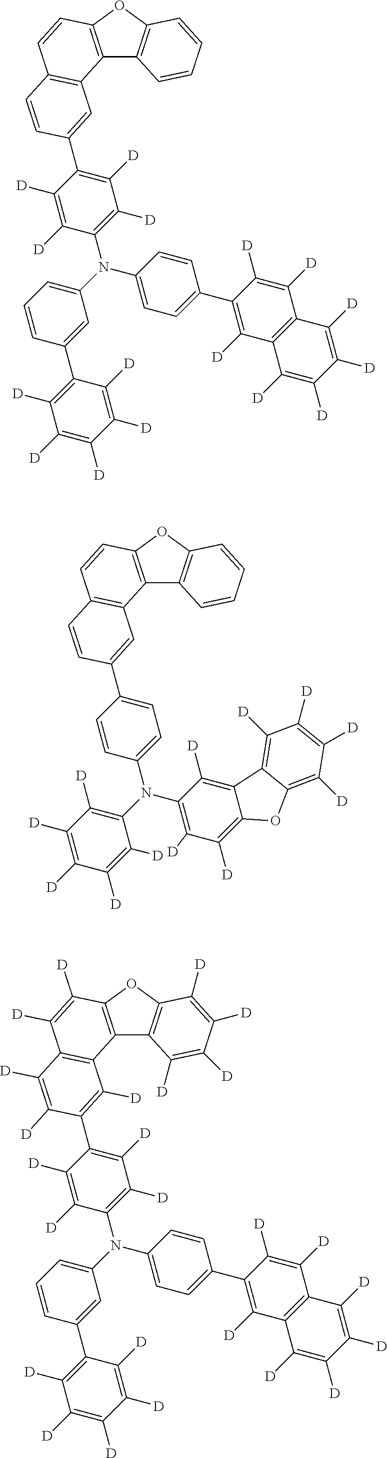

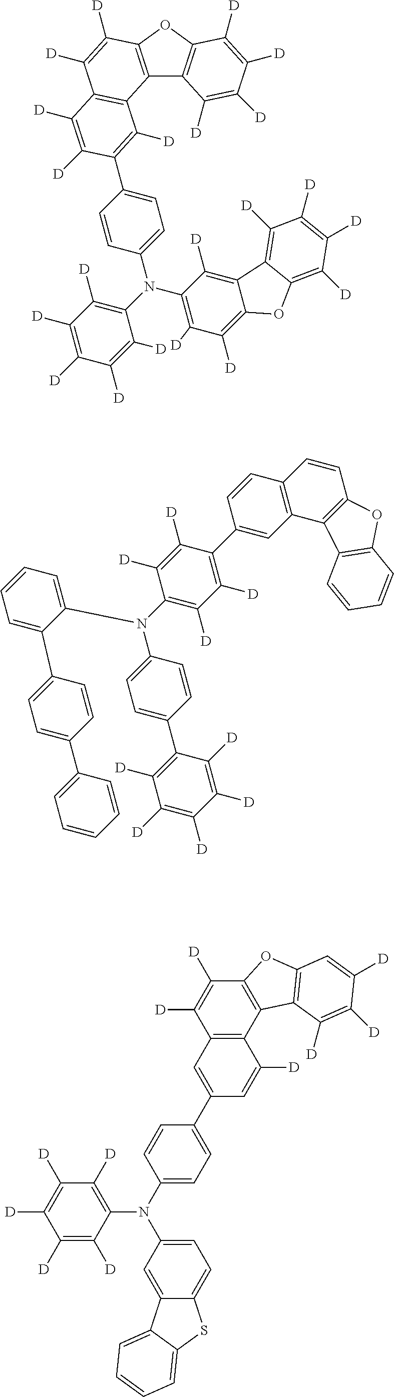 [SMILES: [2H]c1c([2H])c([2H])c(N(c2ccc(-c3c([2H])c([2H])c4c([2H])c([2H])c5oc6c([2H])c([2H])c([2H])c([2H])c6c5c4c3[2H])cc2)c2c([2H])c([2H])c3oc4c([2H])c([2H])c([2H])c([2H])c4c3c2[2H])c([2H])c1[2H] ; [2H]c1c([2H])c([2H])c(-c2ccc(N(c3ccccc3-c3ccc(-c4ccccc4)cc3)c3c([2H])c([2H])c(-c4ccc5ccc6oc7ccccc7c6c5c4)c([2H])c3[2H])cc2)c([2H])c1[2H] ; [2H]c1cc2oc3c([2H])c([2H])c4cc(-c5ccc(N(c6ccc7sc8ccccc8c7c6)c6c([2H])c([2H])c([2H])c([2H])c6[2H])cc5)cc([2H])c4c3c2c([2H])c1[2H]]

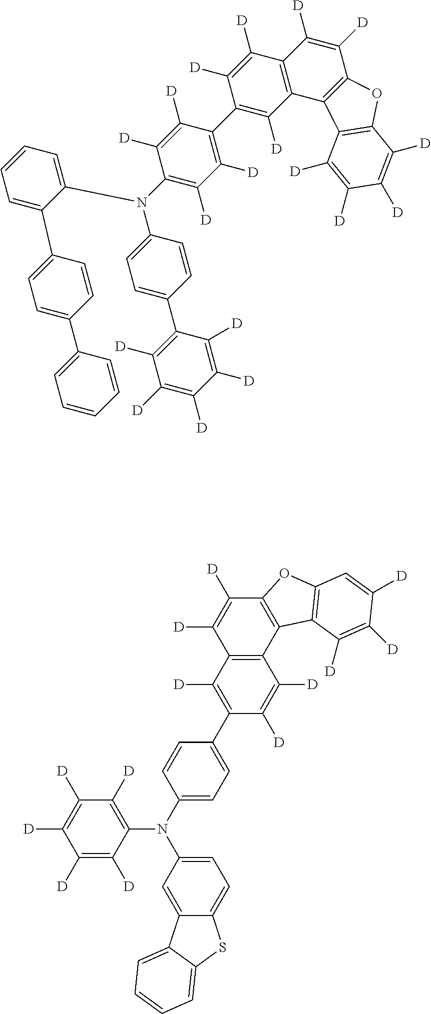 [SMILES: [2H]c1c([2H])c([2H])c(-c2ccc(N(c3ccccc3-c3ccc(-c4ccccc4)cc3)c3c([2H])c([2H])c(-c4c([2H])c([2H])c5c([2H])c([2H])c6oc7c([2H])c([2H])c([2H])c([2H])c7c6c5c4[2H])c([2H])c3[2H])cc2)c([2H])c1[2H] ; [2H]c1cc2oc3c([2H])c([2H])c4c([2H])c(-c5ccc(N(c6ccc7sc8ccccc8c7c6)c6c([2H])c([2H])c([2H])c([2H])c6[2H])cc5)c([2H])c([2H])c4c3c2c([2H])c1[2H]]

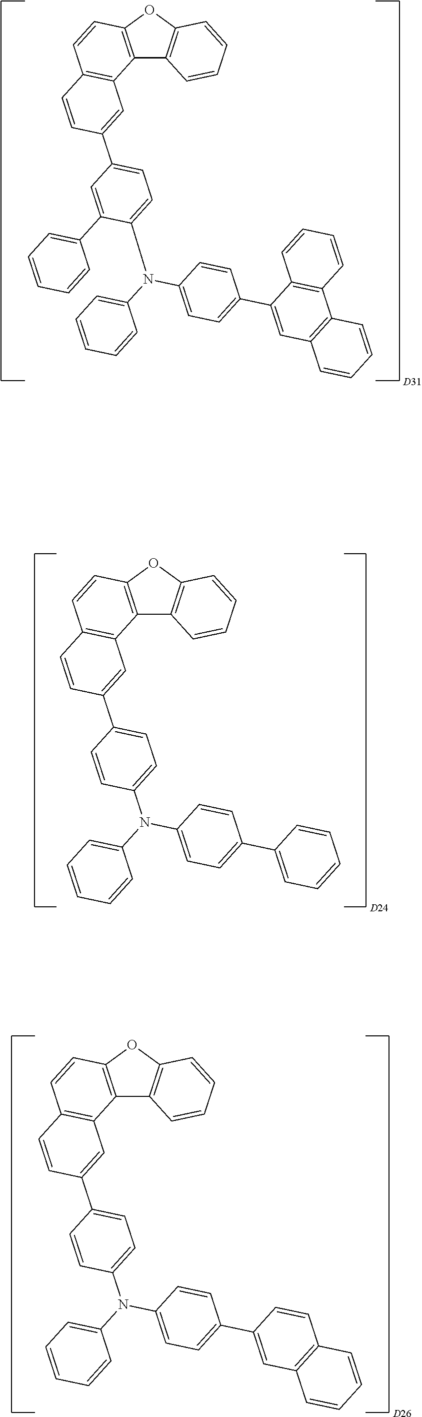

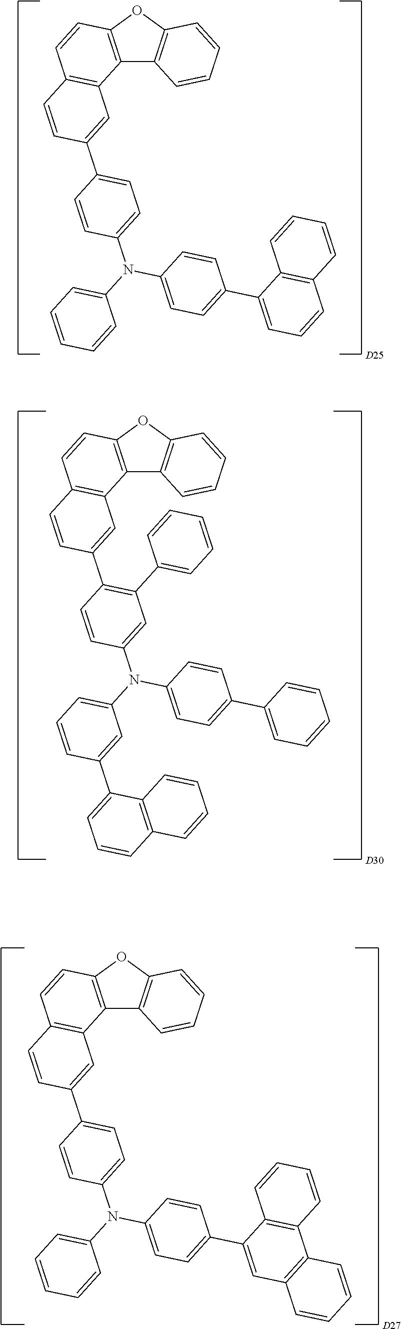

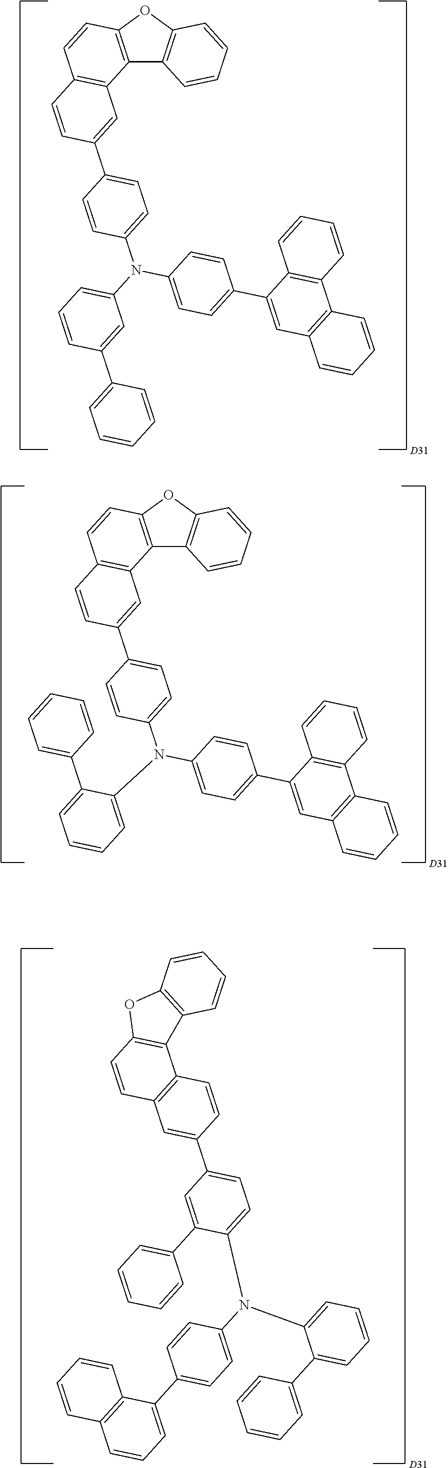

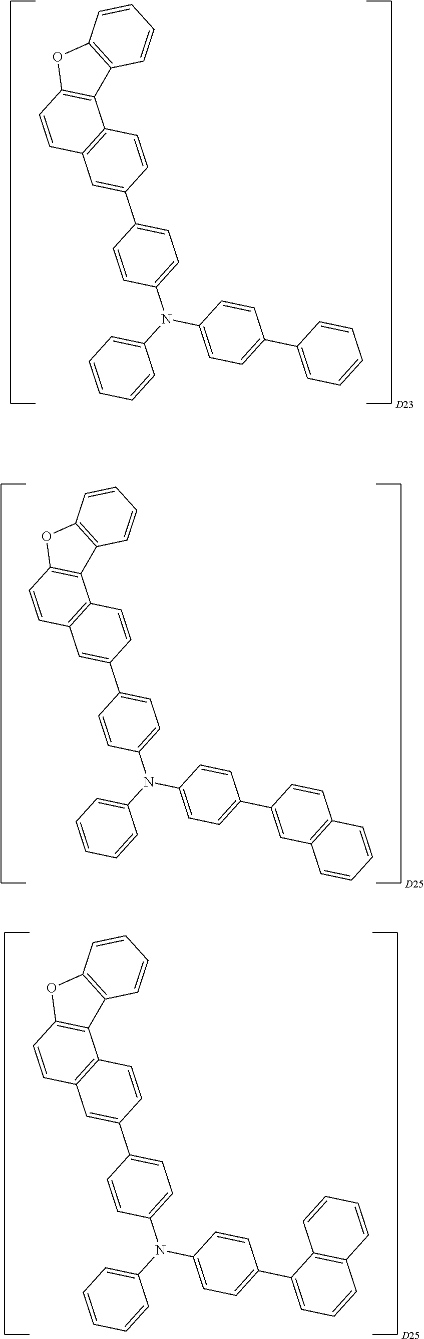

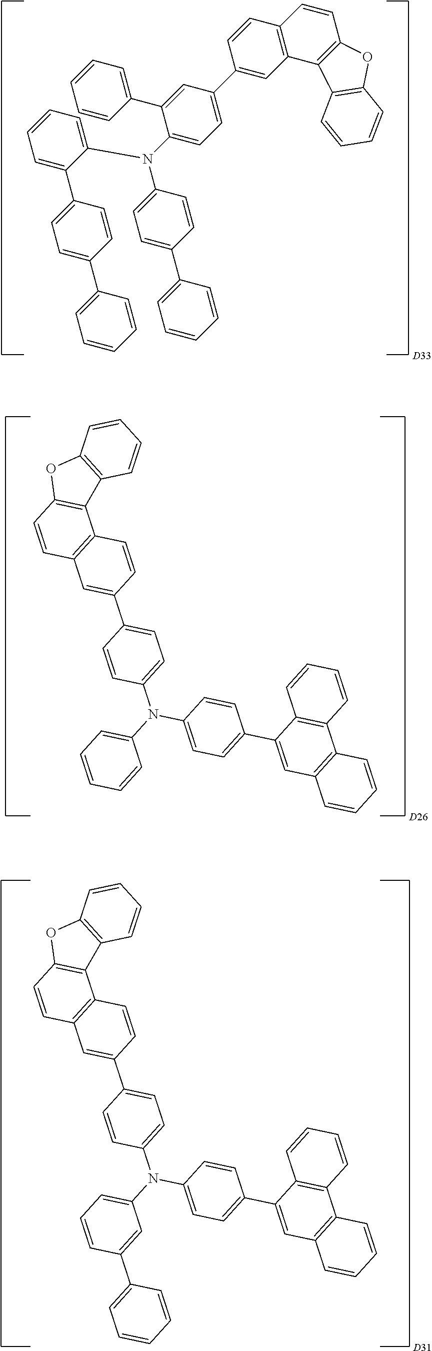

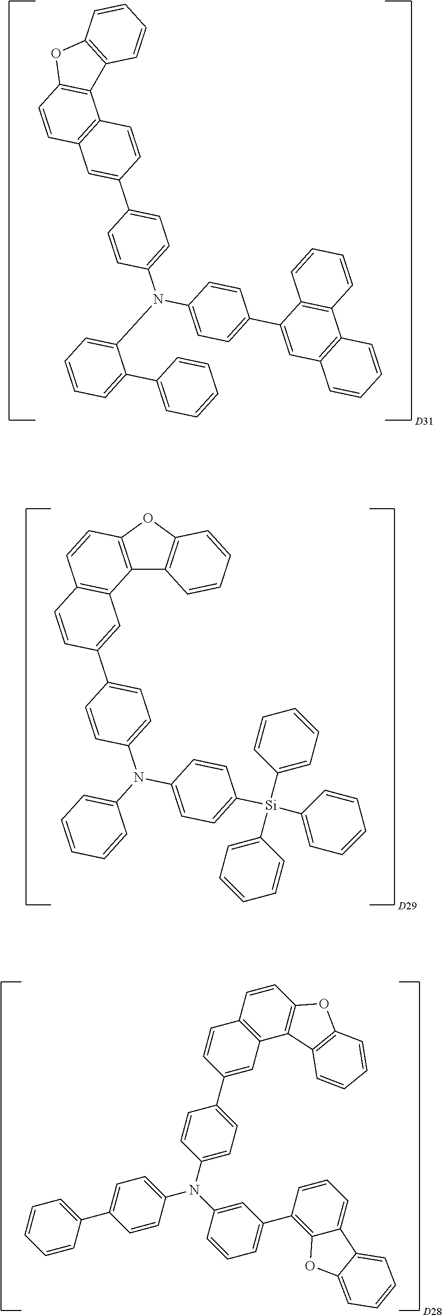

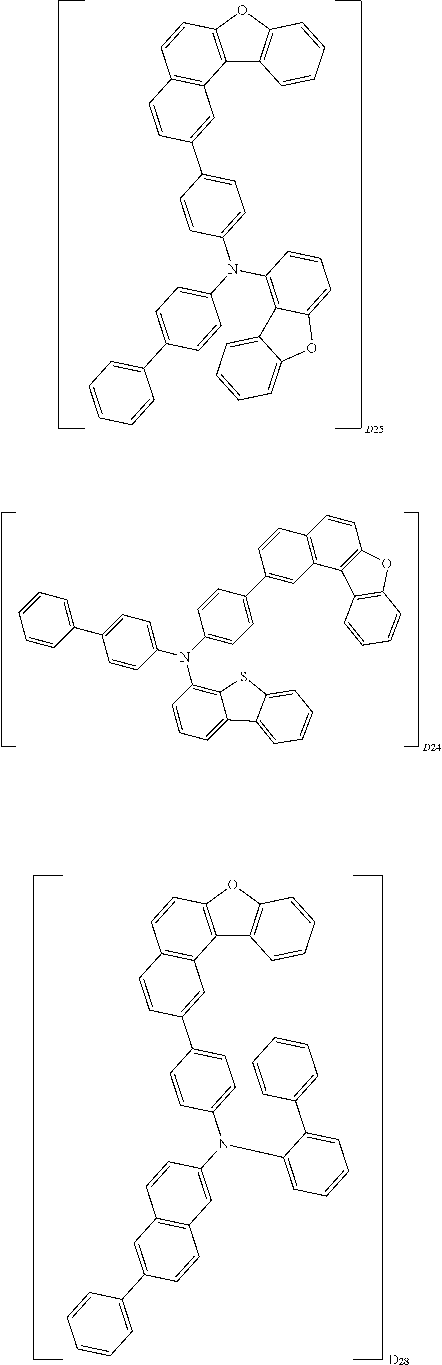

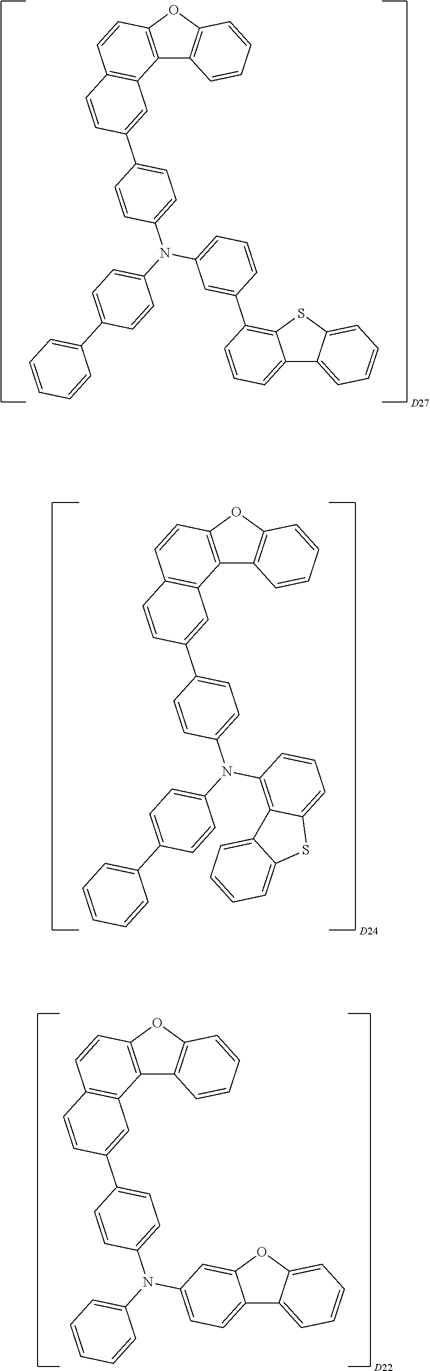

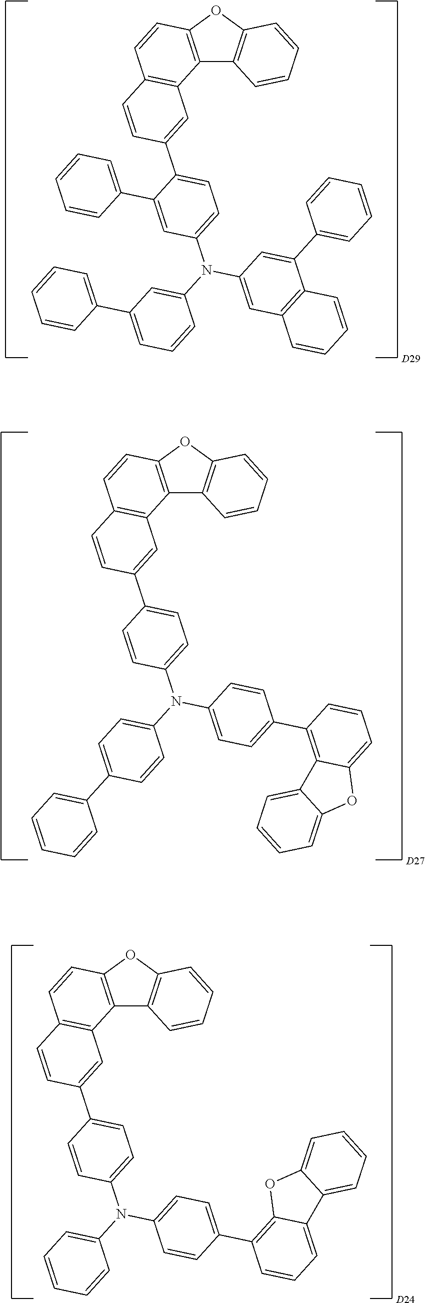

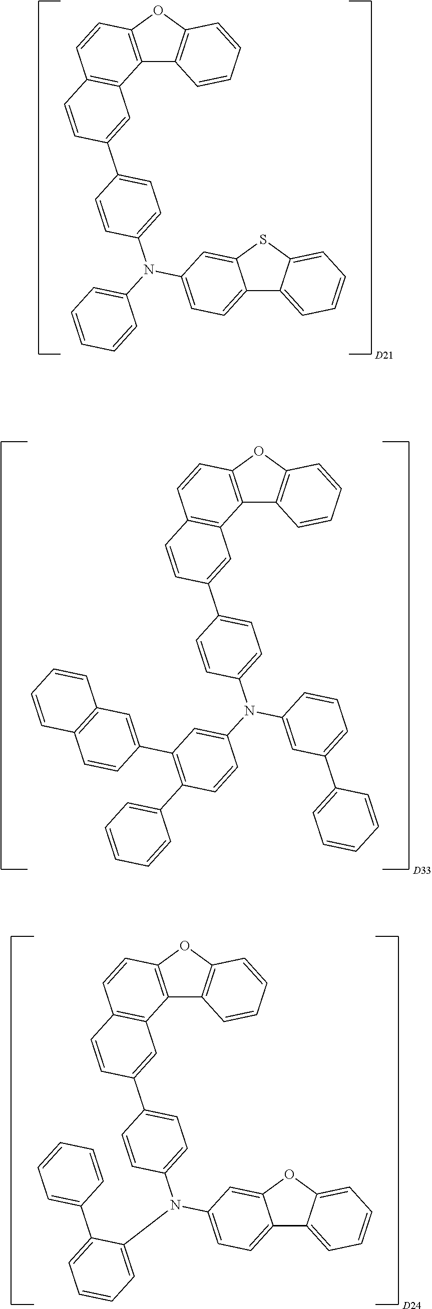

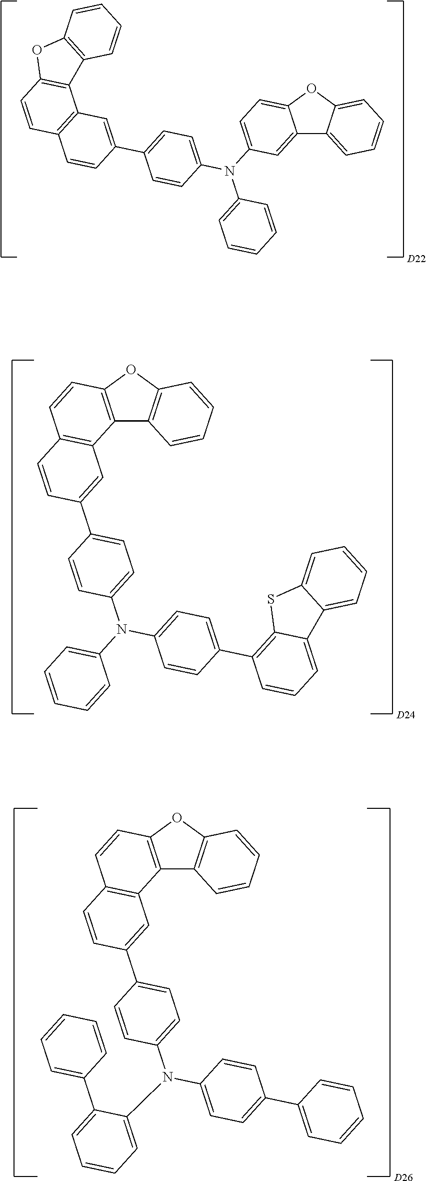

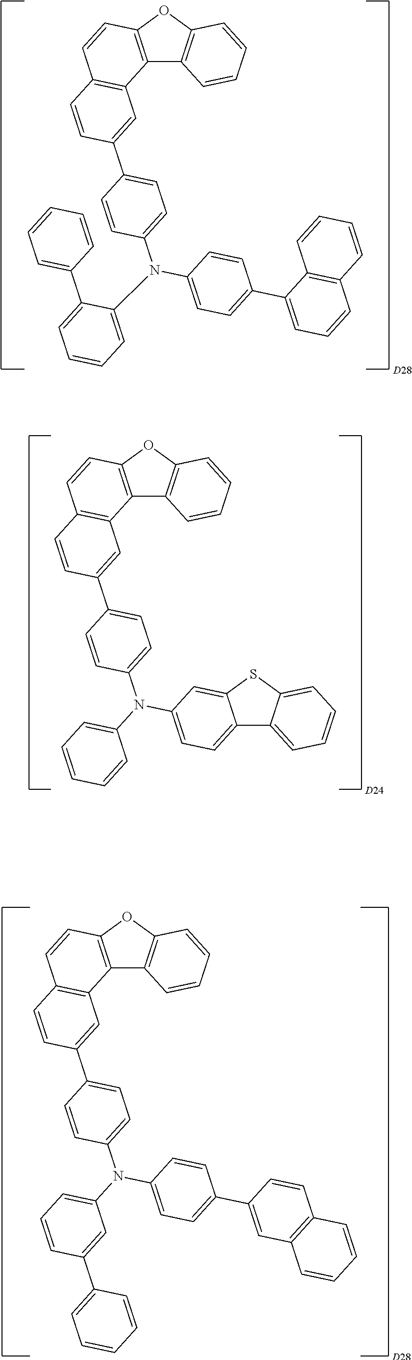

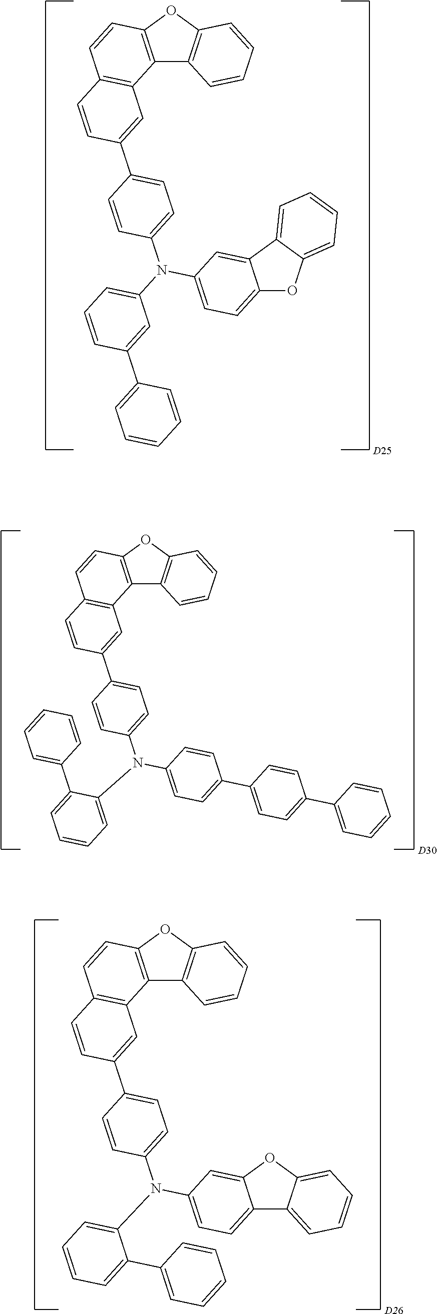

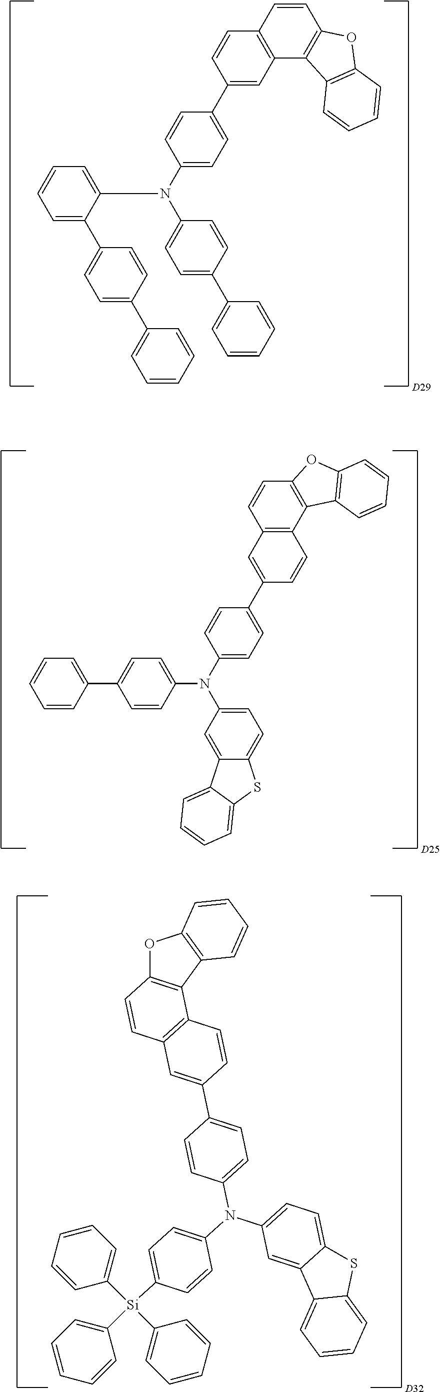

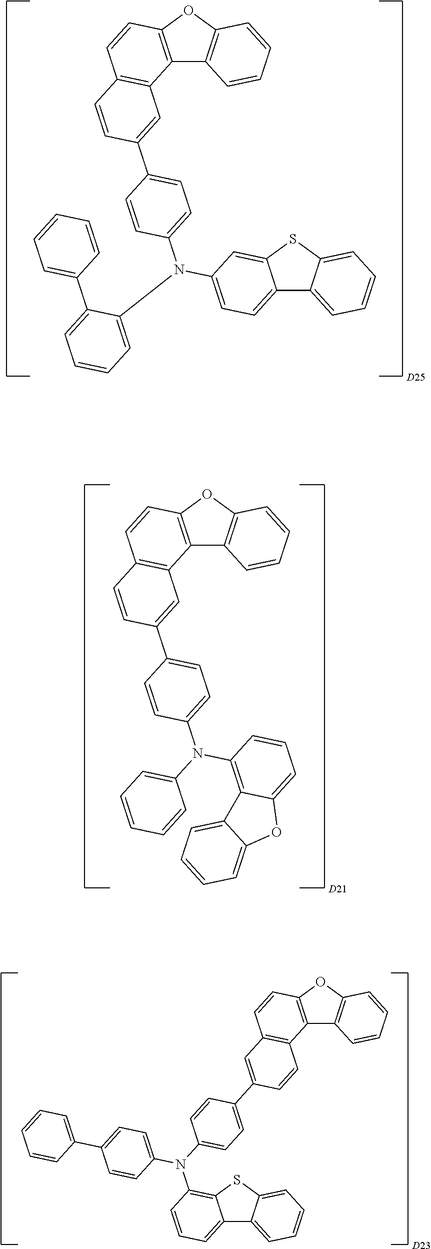

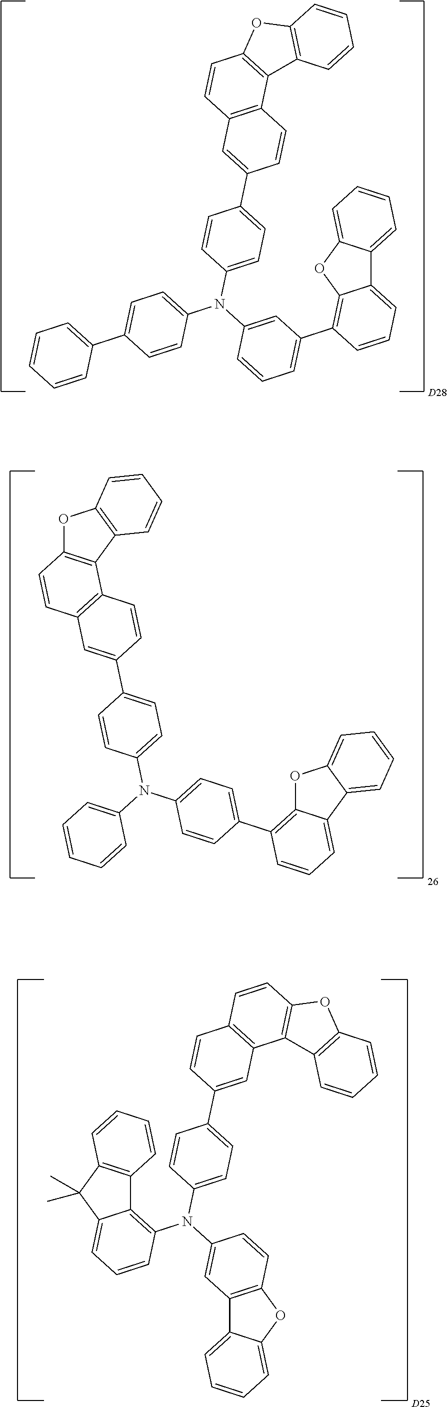

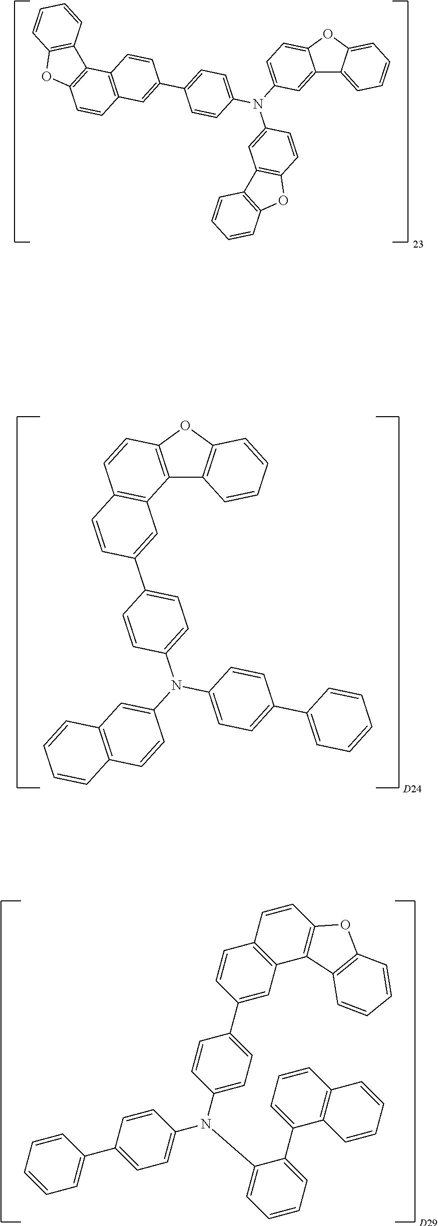

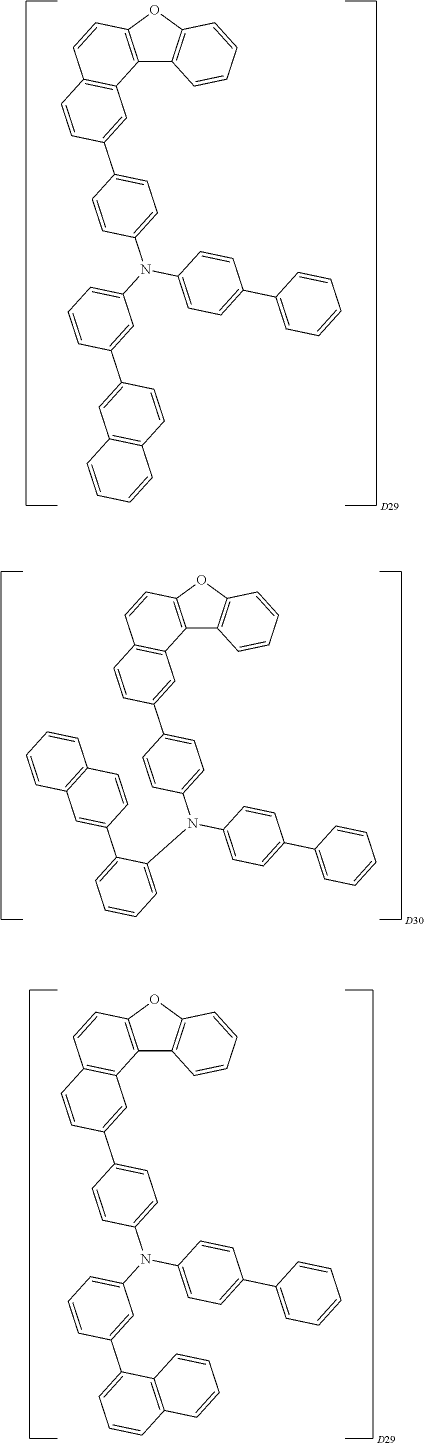

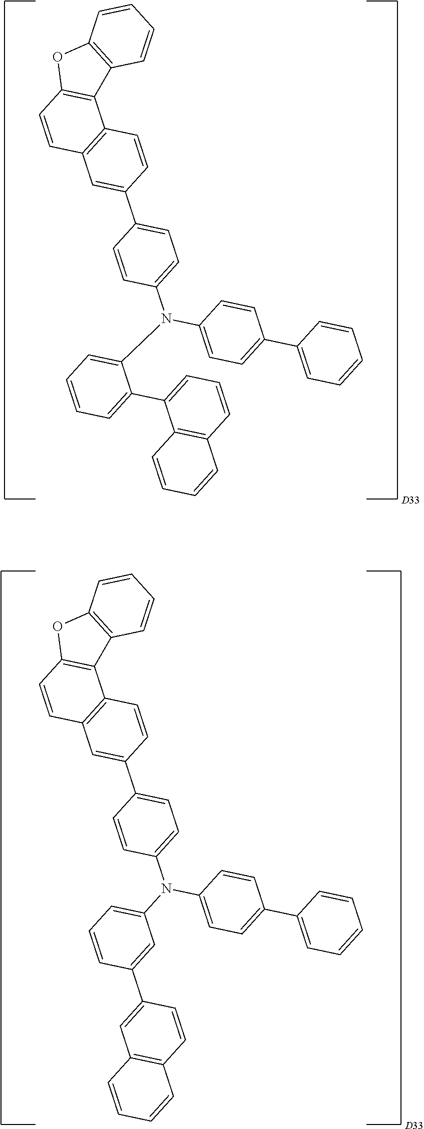

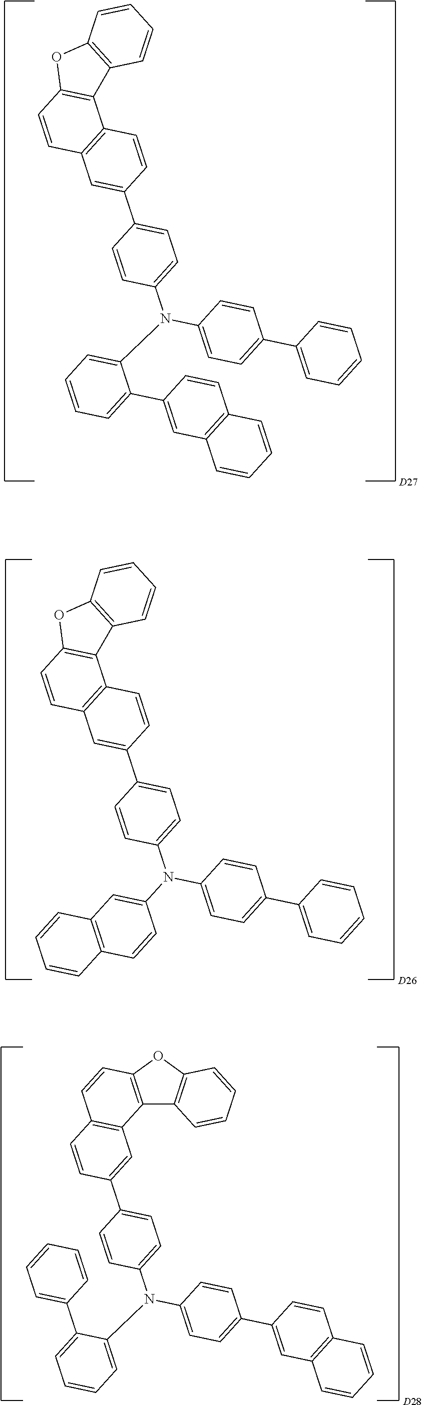

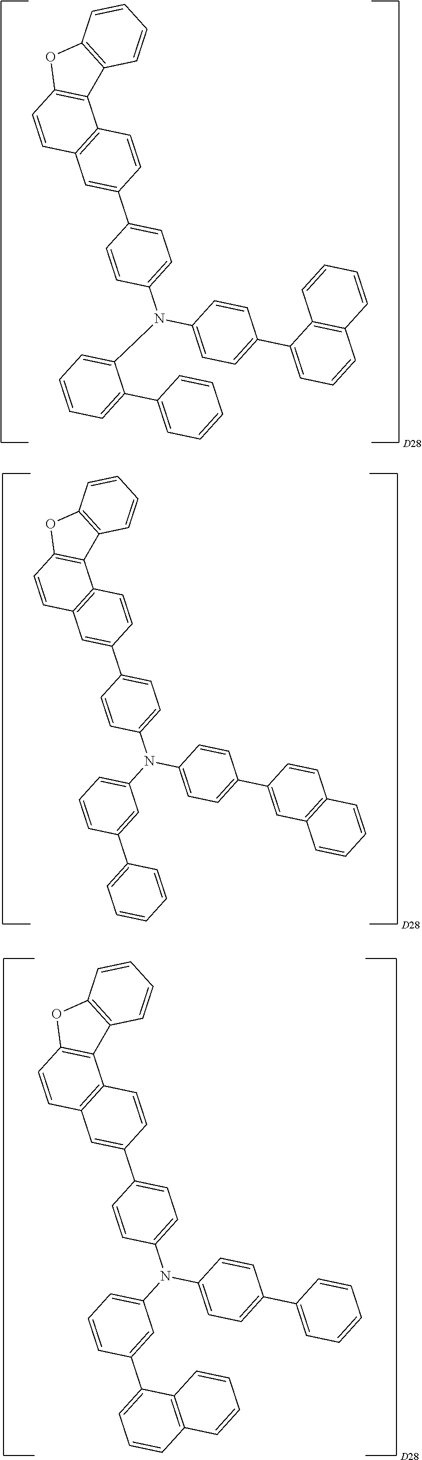

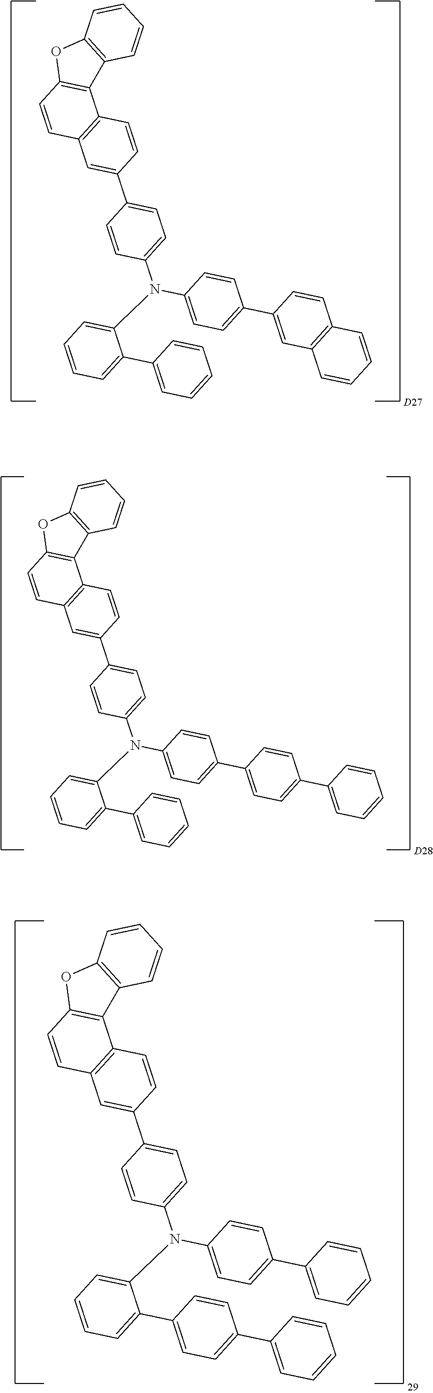

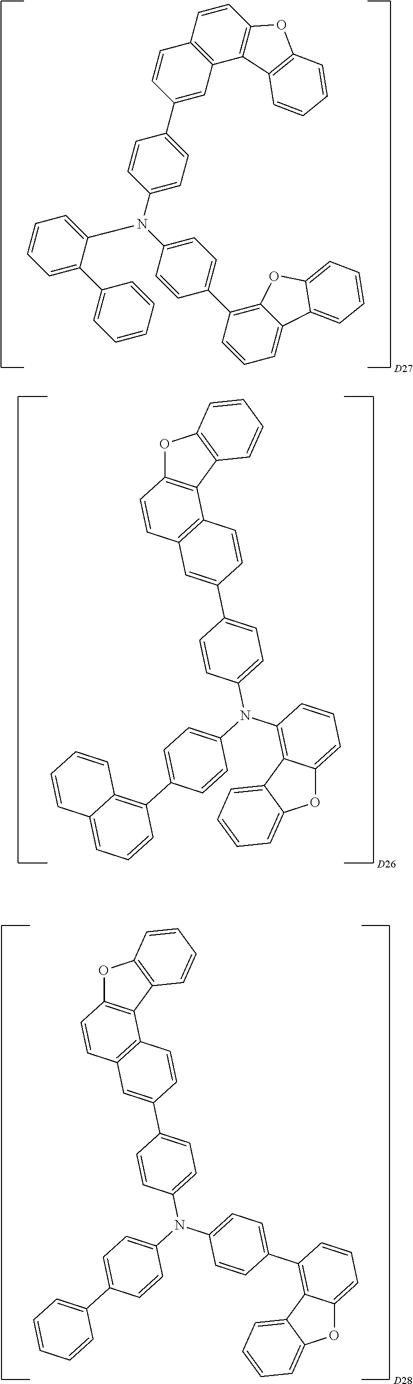

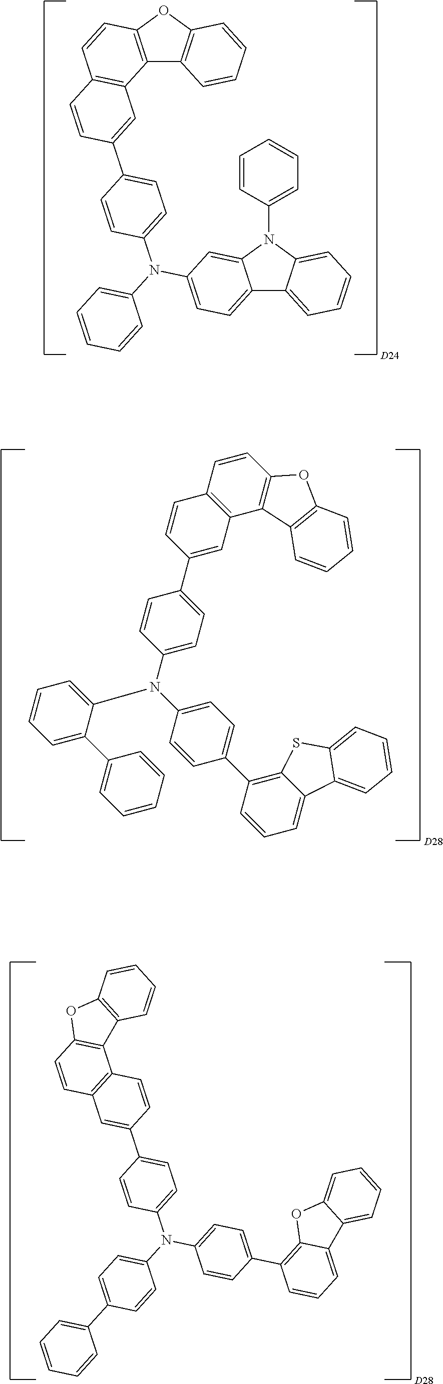

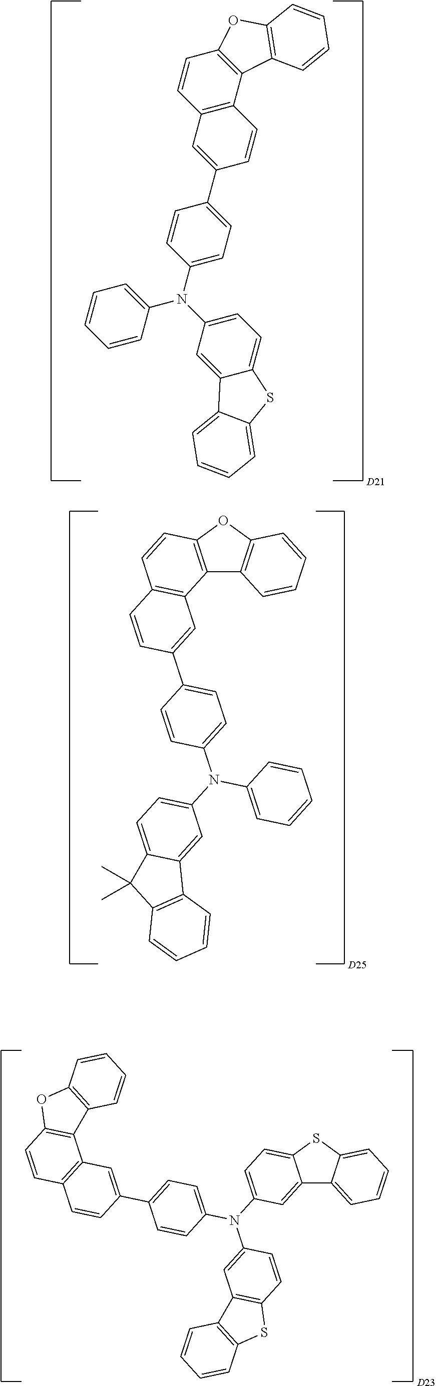

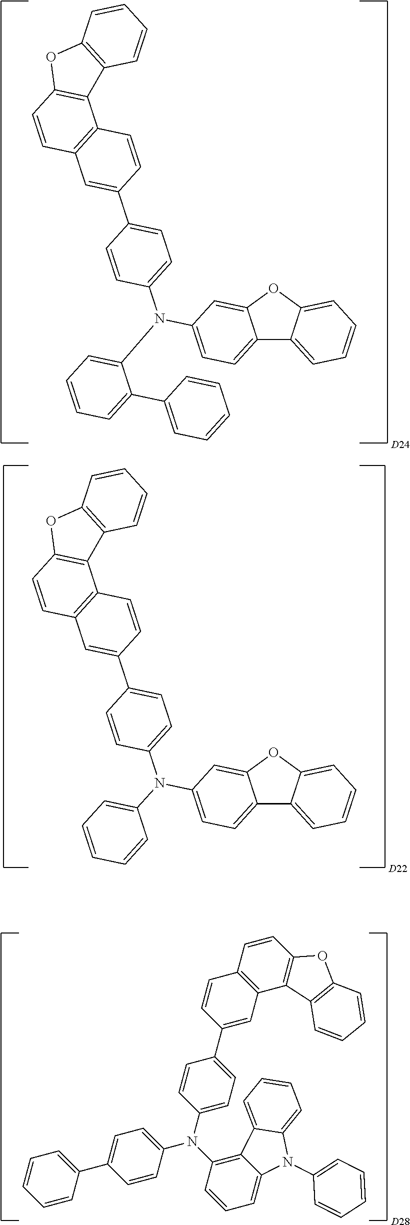

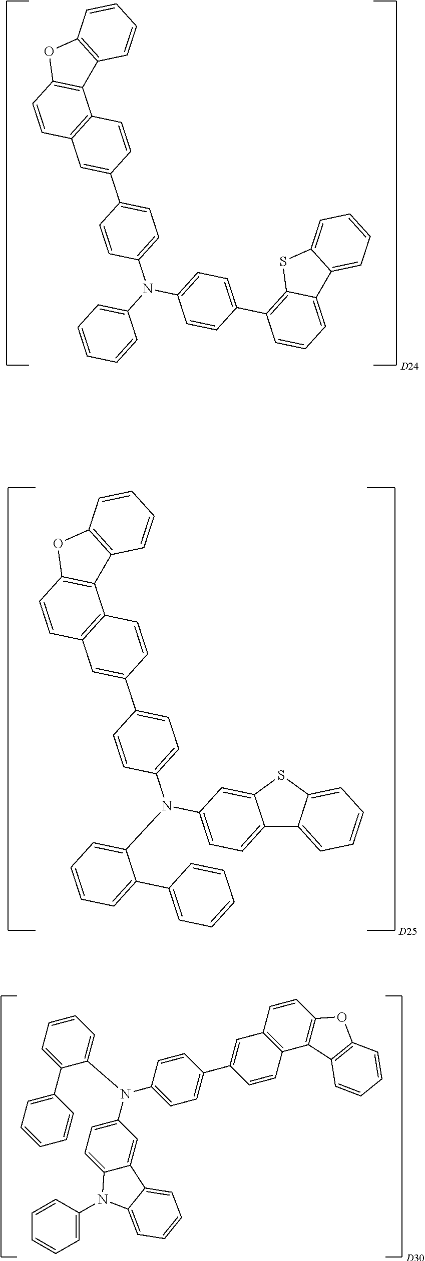

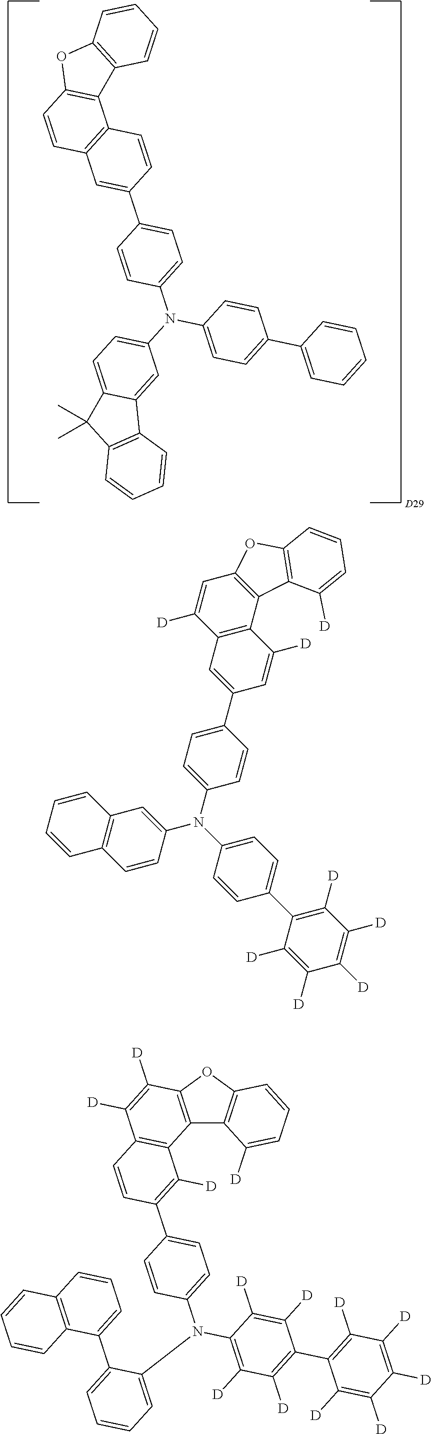

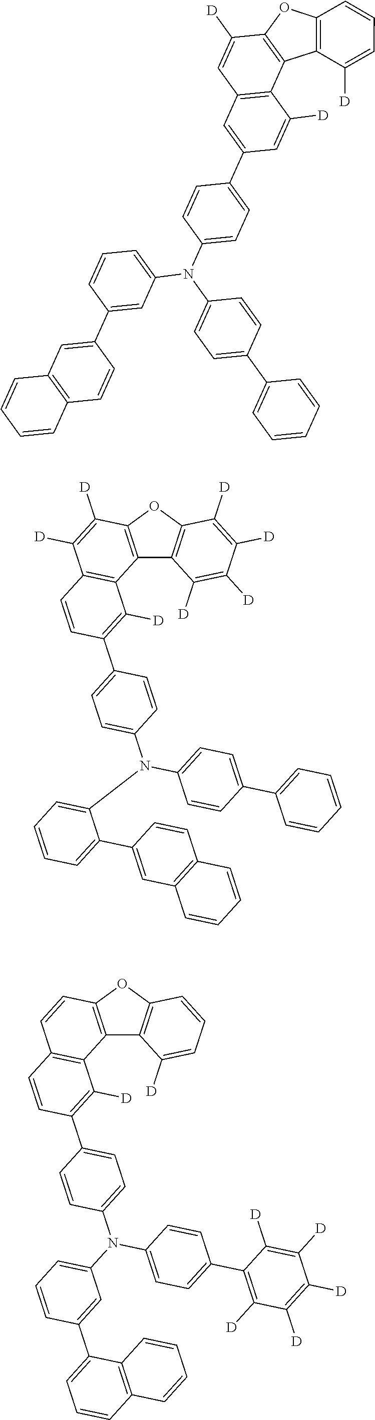 [SMILES: [2H]c1cc2cc(-c3ccc(N(c4ccc(-c5ccccc5)cc4)c4cccc(-c5ccc6ccccc6c5)c4)cc3)cc([2H])c2c2c1oc1cccc([2H])c12 ; [2H]c1c([2H])c([2H])c2c(oc3c([2H])c([2H])c4ccc(-c5ccc(N(c6ccc(-c7ccccc7)cc6)c6ccccc6-c6ccc7ccccc7c6)cc5)c([2H])c4c32)c1[2H] ; [2H]c1c([2H])c([2H])c(-c2ccc(N(c3ccc(-c4ccc5ccc6oc7cccc([2H])c7c6c5c4[2H])cc3)c3cccc(-c4cccc5ccccc45)c3)cc2)c([2H])c1[2H]]

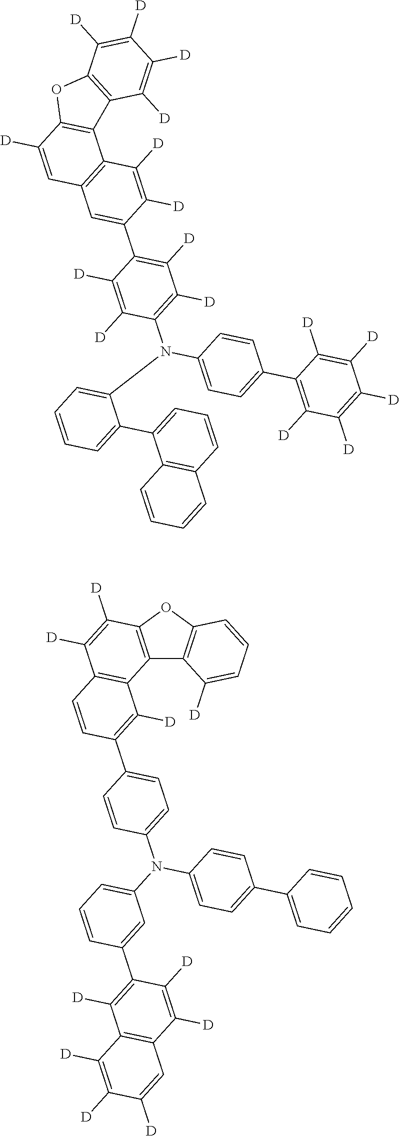 [SMILES: [2H]c1c([2H])c([2H])c(-c2ccc(N(c3ccccc3-c3cccc4ccccc34)c3c([2H])c([2H])c(-c4cc5cc([2H])c6oc7c([2H])c([2H])c([2H])c([2H])c7c6c5c([2H])c4[2H])c([2H])c3[2H])cc2)c([2H])c1[2H] ; [2H]c1cc2c([2H])c([2H])c(-c3cccc(N(c4ccc(-c5ccccc5)cc4)c4ccc(-c5ccc6c([2H])c([2H])c7oc8cccc([2H])c8c7c6c5[2H])cc4)c3)c([2H])c2c([2H])c1[2H]]

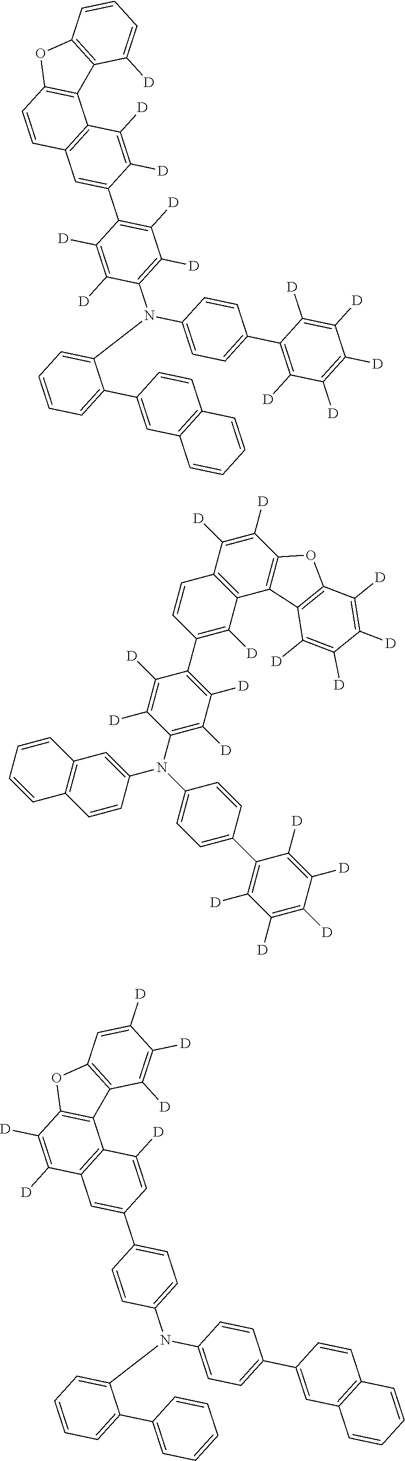 [SMILES: [2H]c1c([2H])c([2H])c(-c2ccc(N(c3ccccc3-c3ccc4ccccc4c3)c3c([2H])c([2H])c(-c4cc5ccc6oc7cccc([2H])c7c6c5c([2H])c4[2H])c([2H])c3[2H])cc2)c([2H])c1[2H] ; [2H]c1c([2H])c([2H])c(-c2ccc(N(c3ccc4ccccc4c3)c3c([2H])c([2H])c(-c4ccc5c([2H])c([2H])c6oc7c([2H])c([2H])c([2H])c([2H])c7c6c5c4[2H])c([2H])c3[2H])cc2)c([2H])c1[2H] ; [2H]c1cc2oc3c([2H])c([2H])c4cc(-c5ccc(N(c6ccc(-c7ccc8ccccc8c7)cc6)c6ccccc6-c6ccccc6)cc5)cc([2H])c4c3c2c([2H])c1[2H]]

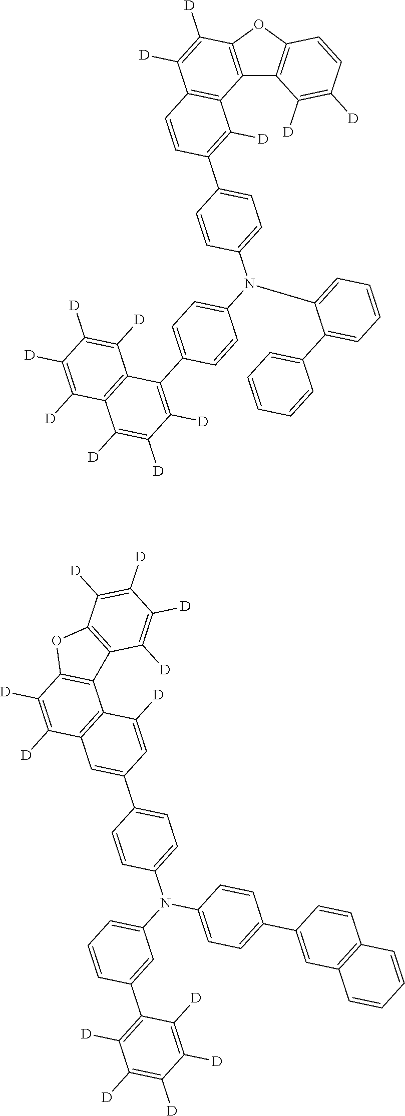 [SMILES: [2H]c1ccc2oc3c([2H])c([2H])c4ccc(-c5ccc(N(c6ccc(-c7c([2H])c([2H])c([2H])c8c([2H])c([2H])c([2H])c([2H])c78)cc6)c6ccccc6-c6ccccc6)cc5)c([2H])c4c3c2c1[2H] ; [2H]c1c([2H])c([2H])c(-c2cccc(N(c3ccc(-c4ccc5ccccc5c4)cc3)c3ccc(-c4cc([2H])c5c(c4)c([2H])c([2H])c4oc6c([2H])c([2H])c([2H])c([2H])c6c45)cc3)c2)c([2H])c1[2H]]

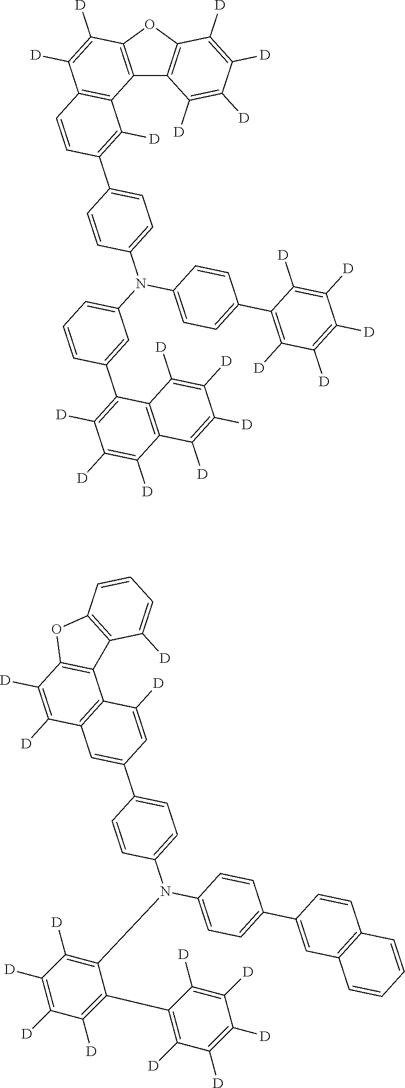 [SMILES: [2H]c1c([2H])c([2H])c(-c2ccc(N(c3ccc(-c4ccc5c([2H])c([2H])c6oc7c([2H])c([2H])c([2H])c([2H])c7c6c5c4[2H])cc3)c3cccc(-c4c([2H])c([2H])c([2H])c5c([2H])c([2H])c([2H])c([2H])c45)c3)cc2)c([2H])c1[2H] ; [2H]c1c([2H])c([2H])c(-c2c([2H])c([2H])c([2H])c([2H])c2N(c2ccc(-c3ccc4ccccc4c3)cc2)c2ccc(-c3cc([2H])c4c(c3)c([2H])c([2H])c3oc5cccc([2H])c5c34)cc2)c([2H])c1[2H]]

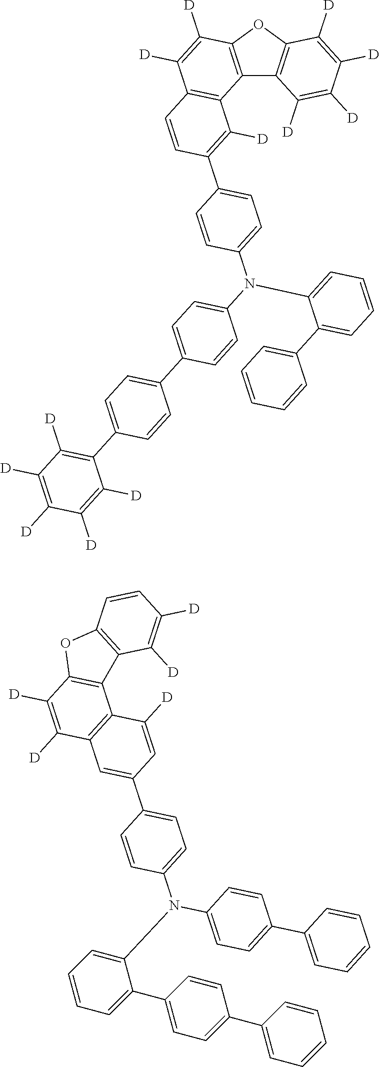 [SMILES: [2H]c1c([2H])c([2H])c(-c2ccc(-c3ccc(N(c4ccc(-c5ccc6c([2H])c([2H])c7oc8c([2H])c([2H])c([2H])c([2H])c8c7c6c5[2H])cc4)c4ccccc4-c4ccccc4)cc3)cc2)c([2H])c1[2H] ; [2H]c1ccc2oc3c([2H])c([2H])c4cc(-c5ccc(N(c6ccc(-c7ccccc7)cc6)c6ccccc6-c6ccc(-c7ccccc7)cc6)cc5)cc([2H])c4c3c2c1[2H]]

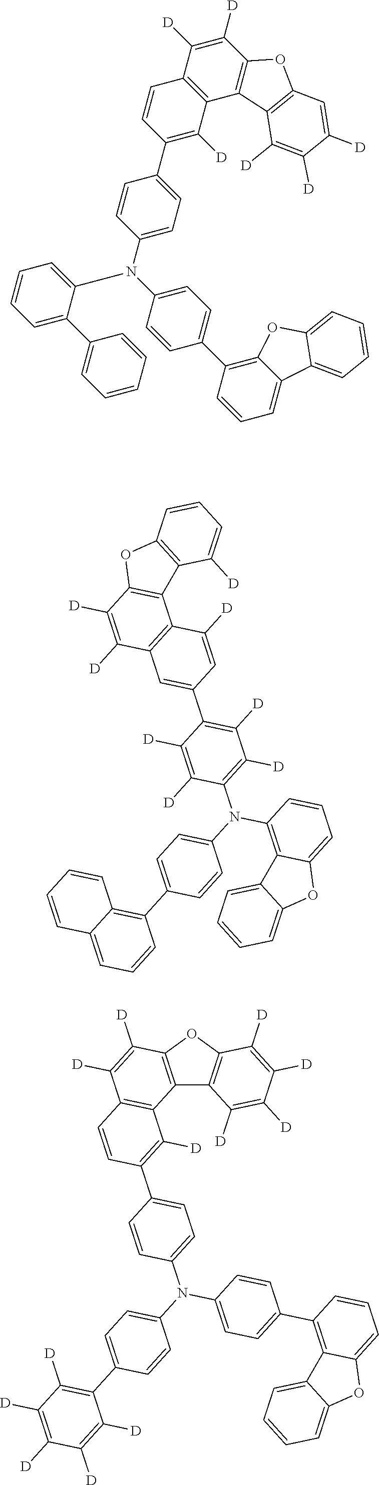

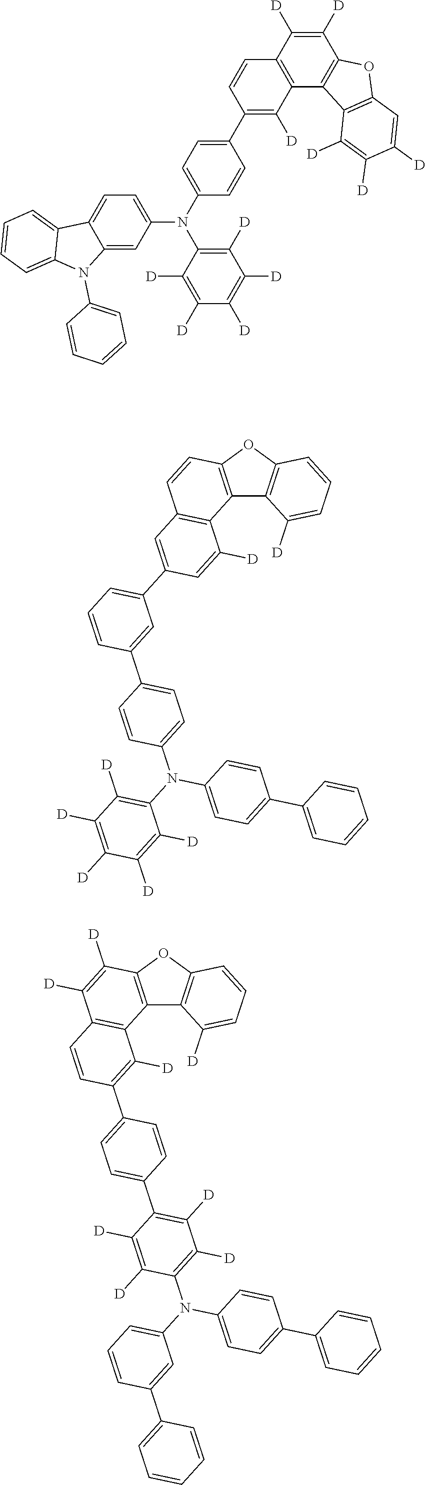 [SMILES: [2H]c1cc2oc3c([2H])c([2H])c4ccc(-c5ccc(N(c6ccc7c8ccccc8n(-c8ccccc8)c7c6)c6c([2H])c([2H])c([2H])c([2H])c6[2H])cc5)c([2H])c4c3c2c([2H])c1[2H] ; [2H]c1c([2H])c([2H])c(N(c2ccc(-c3ccccc3)cc2)c2ccc(-c3cccc(-c4cc([2H])c5c(ccc6oc7cccc([2H])c7c65)c4)c3)cc2)c([2H])c1[2H] ; [2H]c1c([2H])c(N(c2ccc(-c3ccccc3)cc2)c2cccc(-c3ccccc3)c2)c([2H])c([2H])c1-c1ccc(-c2ccc3c([2H])c([2H])c4oc5cccc([2H])c5c4c3c2[2H])cc1]

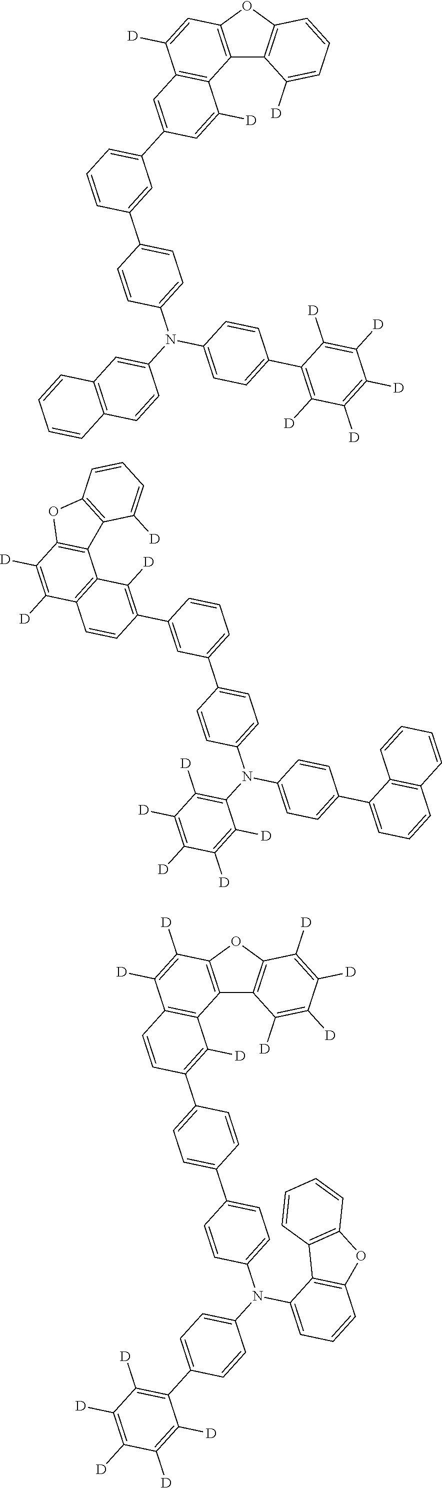

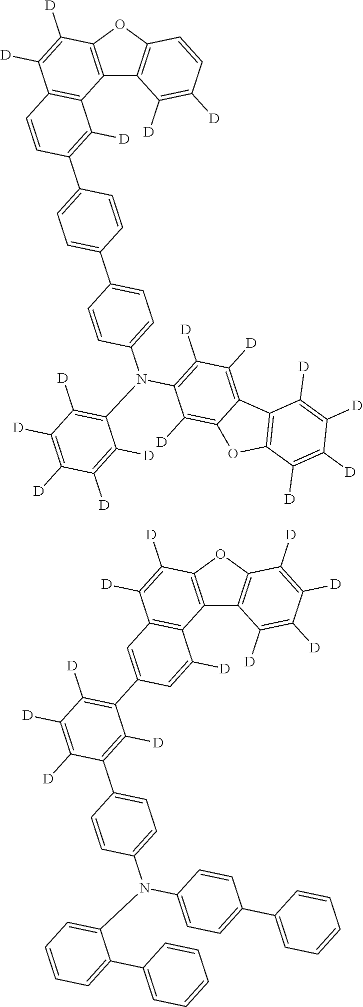

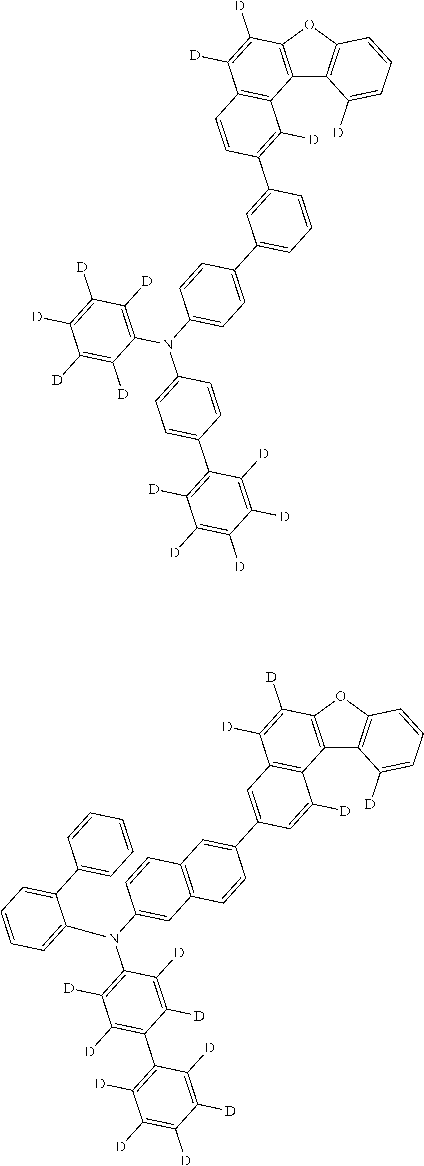 [SMILES: [2H]c1c([2H])c([2H])c(-c2ccc(N(c3ccc(-c4cccc(-c5ccc6c([2H])c([2H])c7oc8cccc([2H])c8c7c6c5[2H])c4)cc3)c3c([2H])c([2H])c([2H])c([2H])c3[2H])cc2)c([2H])c1[2H] ; [2H]c1c([2H])c([2H])c(-c2c([2H])c([2H])c(N(c3ccc4cc(-c5cc([2H])c6c(c5)c([2H])c([2H])c5oc7cccc([2H])c7c56)ccc4c3)c3ccccc3-c3ccccc3)c([2H])c2[2H])c([2H])c1[2H]]

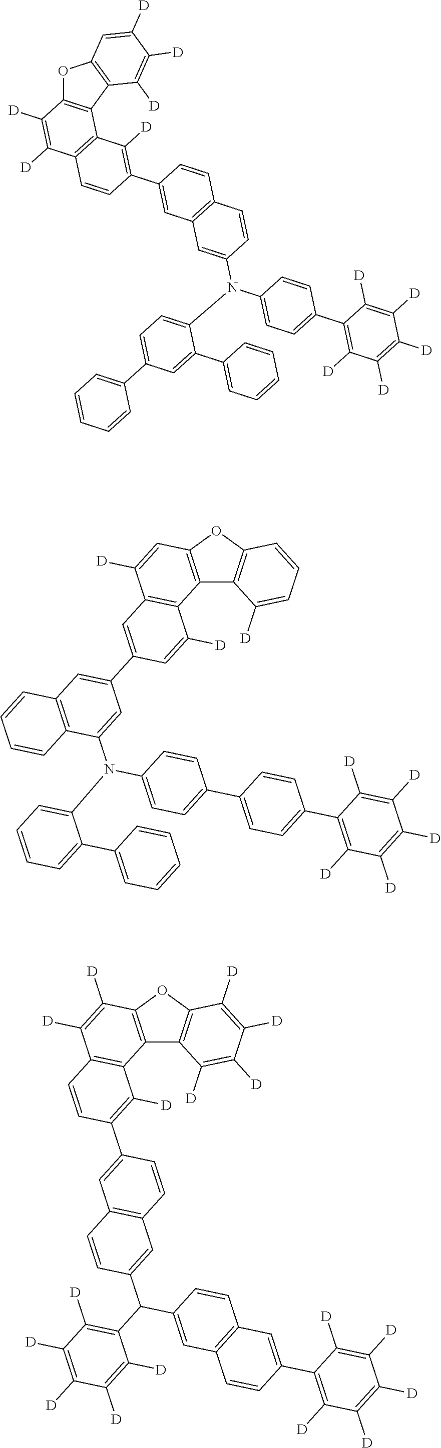

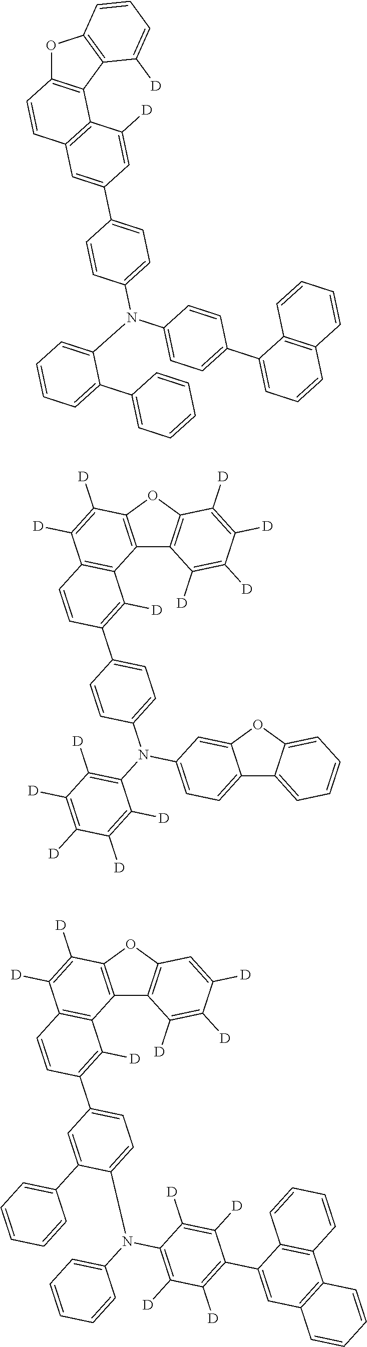

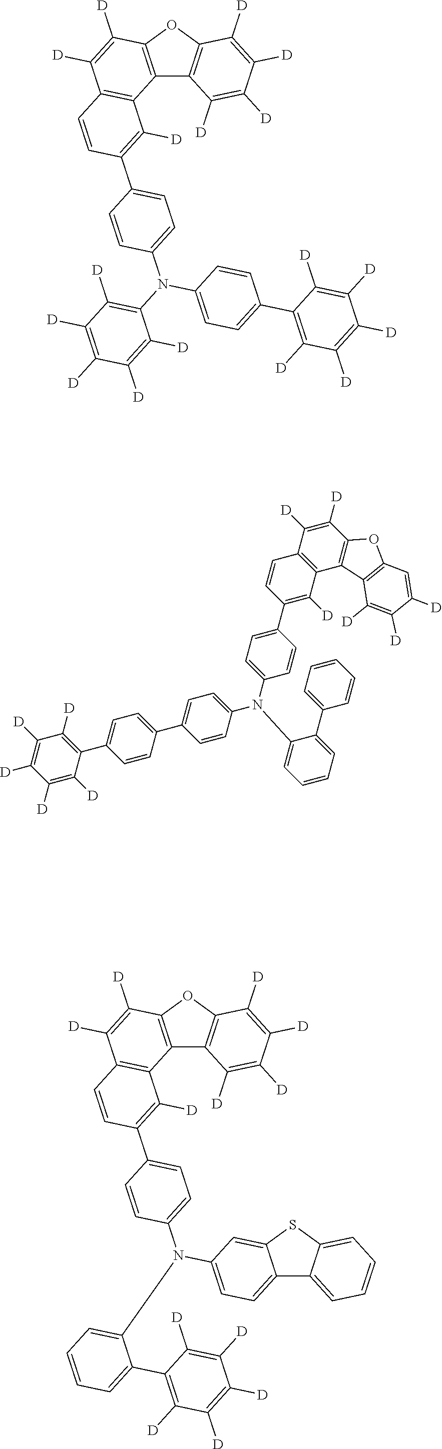

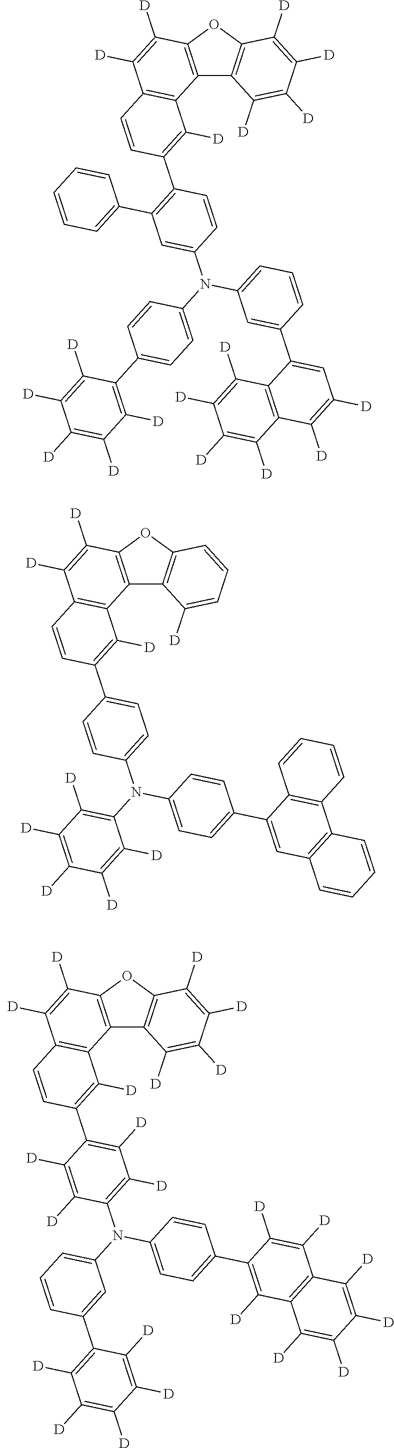

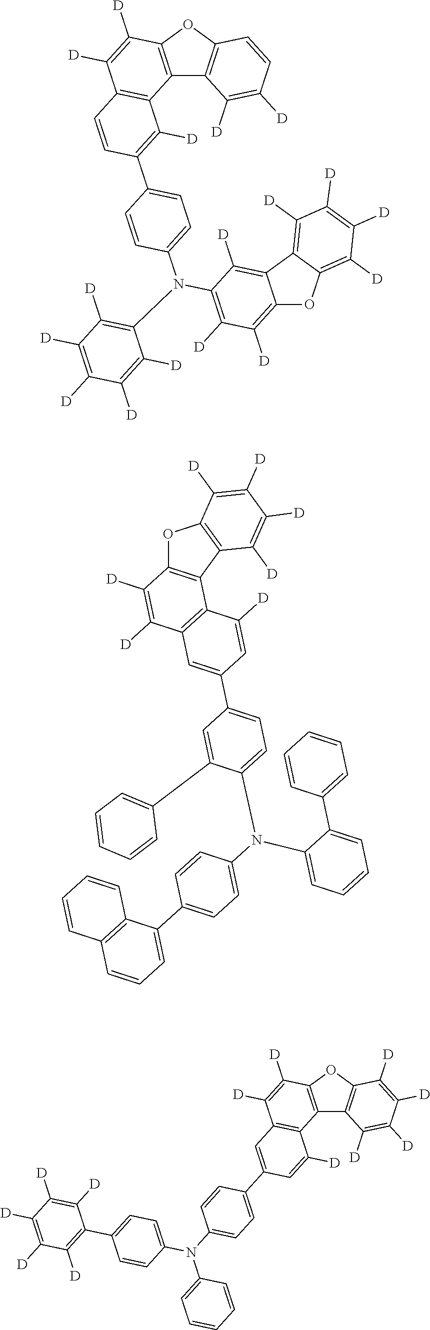

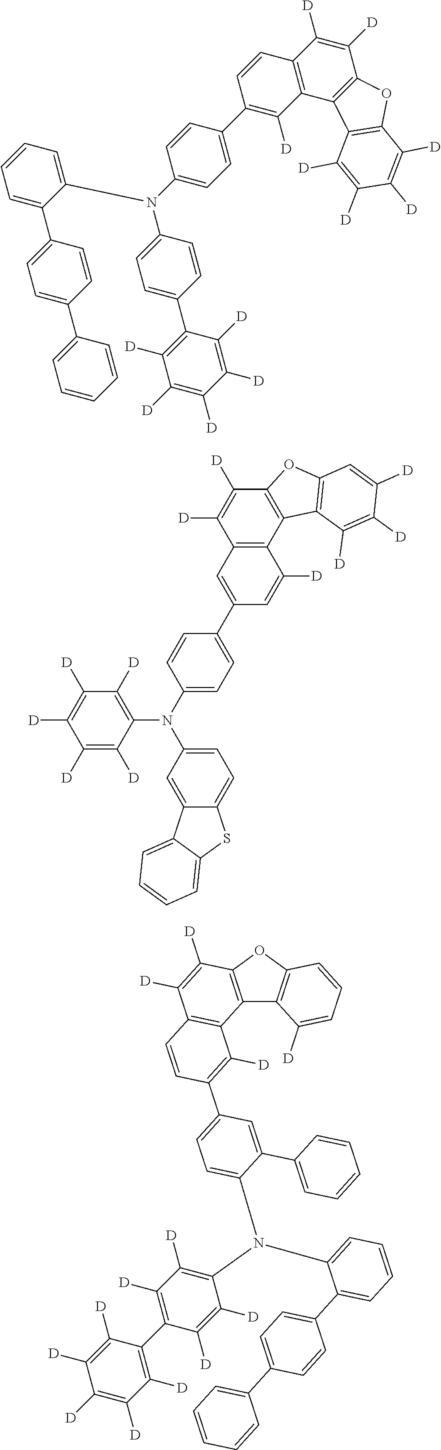

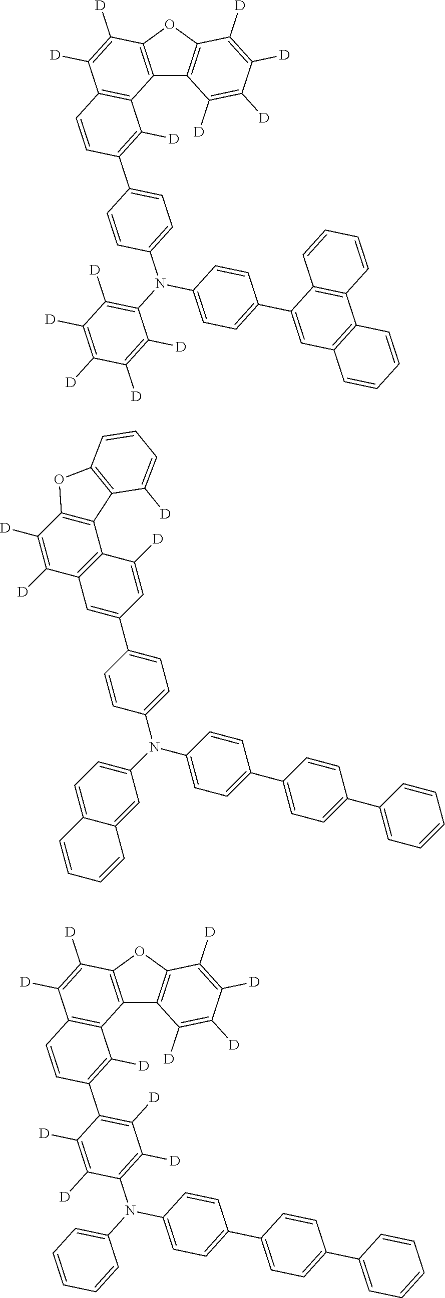

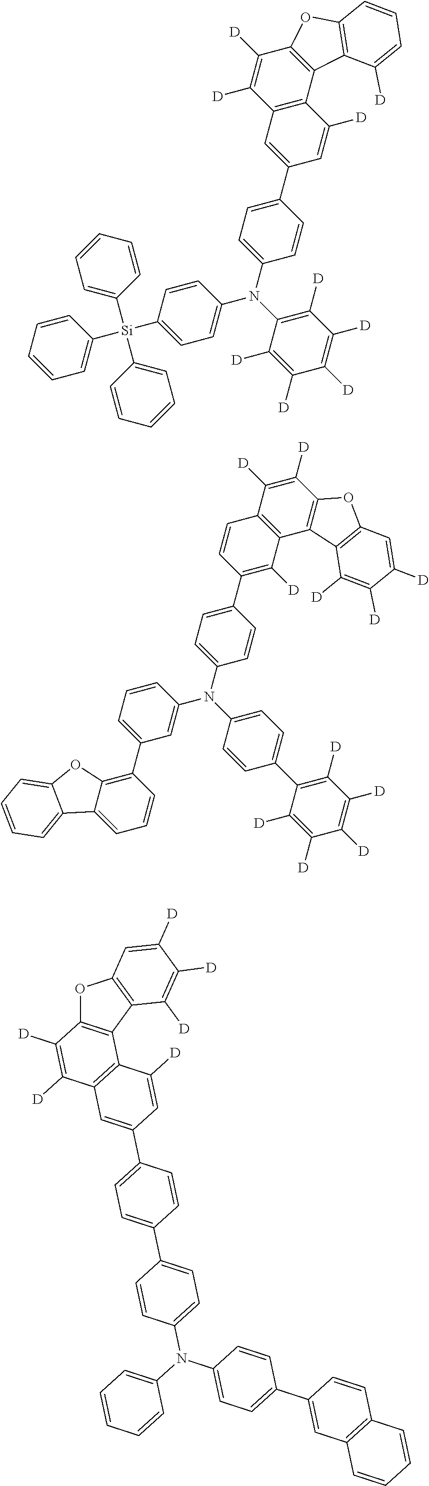

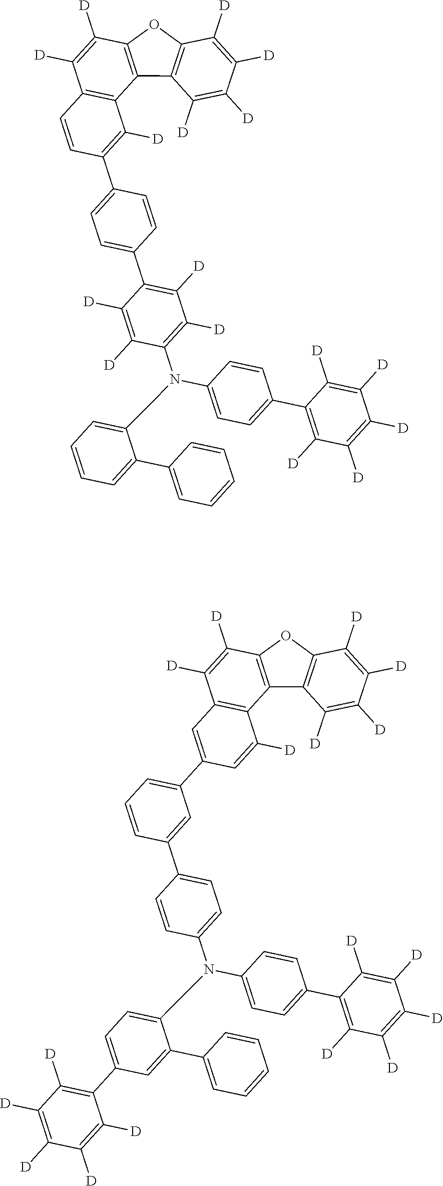 [SMILES: [2H]c1c([2H])c([2H])c(-c2ccc(N(c3ccccc3-c3ccccc3)c3c([2H])c([2H])c(-c4ccc(-c5ccc6c([2H])c([2H])c7oc8c([2H])c([2H])c([2H])c([2H])c8c7c6c5[2H])cc4)c([2H])c3[2H])cc2)c([2H])c1[2H] ; [2H]c1c([2H])c([2H])c(-c2ccc(N(c3ccc(-c4cccc(-c5cc([2H])c6c(c5)c([2H])c([2H])c5oc7c([2H])c([2H])c([2H])c([2H])c7c56)c4)cc3)c3ccc(-c4c([2H])c([2H])c([2H])c([2H])c4[2H])cc3-c3ccccc3)cc2)c([2H])c1[2H]]

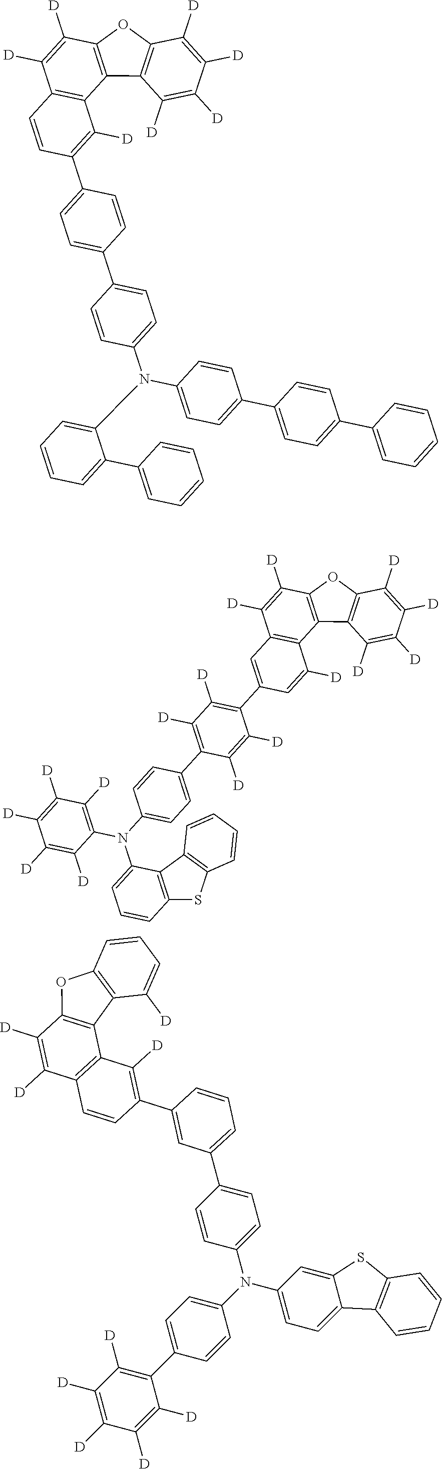

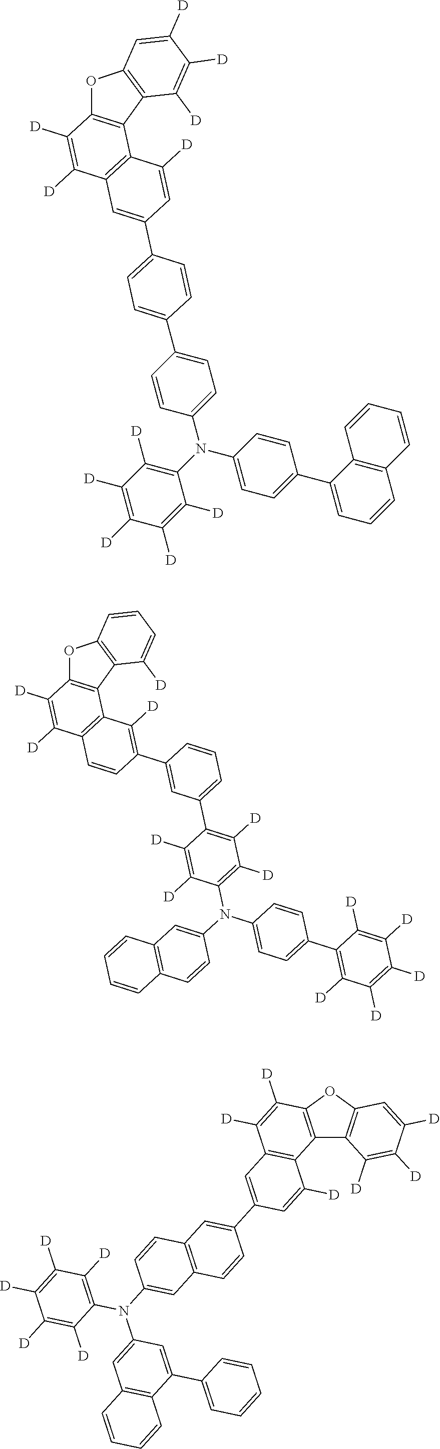 [SMILES: [2H]c1cc2oc3c([2H])c([2H])c4cc(-c5ccc(-c6ccc(N(c7ccc(-c8cccc9ccccc89)cc7)c7c([2H])c([2H])c([2H])c([2H])c7[2H])cc6)cc5)cc([2H])c4c3c2c([2H])c1[2H] ; [2H]c1c([2H])c([2H])c(-c2ccc(N(c3ccc4ccccc4c3)c3c([2H])c([2H])c(-c4cccc(-c5ccc6c([2H])c([2H])c7oc8cccc([2H])c8c7c6c5[2H])c4)c([2H])c3[2H])cc2)c([2H])c1[2H] ; [2H]c1cc2oc3c([2H])c([2H])c4cc(-c5ccc6cc(N(c7cc(-c8ccccc8)c8ccccc8c7)c7c([2H])c([2H])c([2H])c([2H])c7[2H])ccc6c5)cc([2H])c4c3c2c([2H])c1[2H]]

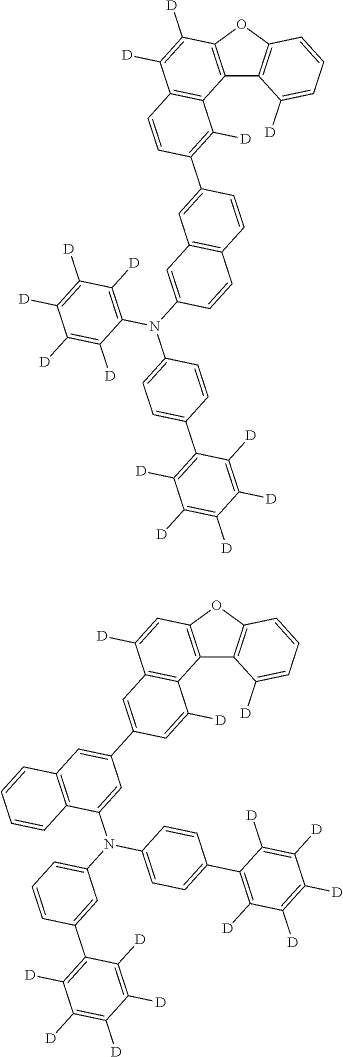 [SMILES: [2H]c1c([2H])c([2H])c(-c2ccc(N(c3ccc4ccc(-c5ccc6c([2H])c([2H])c7oc8cccc([2H])c8c7c6c5[2H])cc4c3)c3c([2H])c([2H])c([2H])c([2H])c3[2H])cc2)c([2H])c1[2H] ; [2H]c1c([2H])c([2H])c(-c2ccc(N(c3cccc(-c4c([2H])c([2H])c([2H])c([2H])c4[2H])c3)c3cc(-c4cc([2H])c5c(c4)c([2H])cc4oc6cccc([2H])c6c45)cc4ccccc34)cc2)c([2H])c1[2H]]

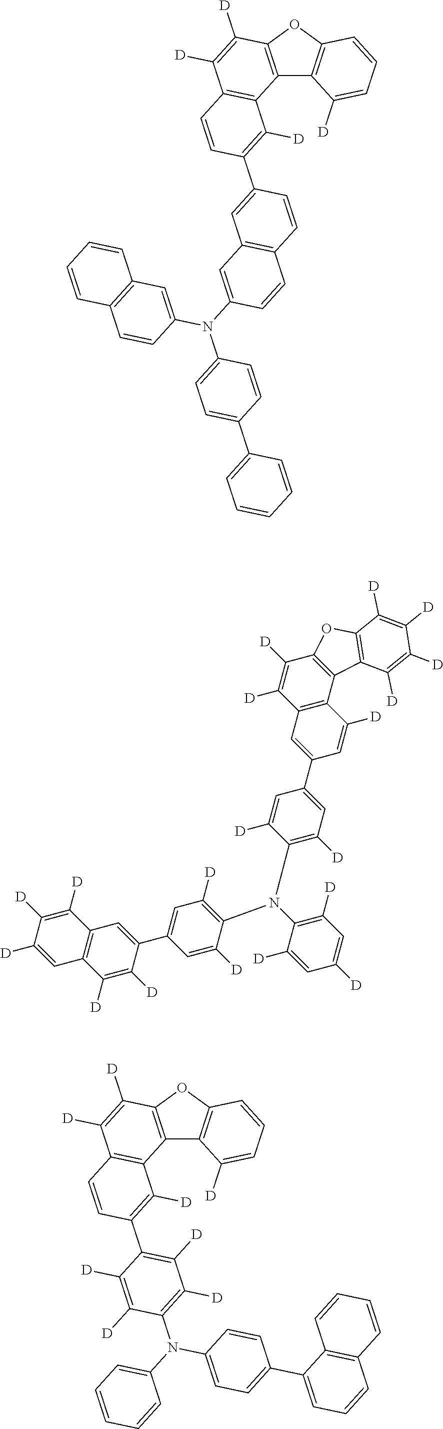 [SMILES: [2H]c1c([2H])c2oc3cccc([2H])c3c2c2c([2H])c(-c3ccc4ccc(N(c5ccc(-c6ccccc6)cc5)c5ccc6ccccc6c5)cc4c3)ccc12 ; [2H]c1cc([2H])c(N(c2c([2H])cc(-c3cc([2H])c4c(c3)c([2H])c([2H])c3oc5c([2H])c([2H])c([2H])c([2H])c5c34)cc2[2H])c2c([2H])cc(-c3cc4c([2H])c([2H])c([2H])cc4c([2H])c3[2H])cc2[2H])c([2H])c1 ; [2H]c1c([2H])c(N(c2ccccc2)c2ccc(-c3cccc4ccccc34)cc2)c([2H])c([2H])c1-c1ccc2c([2H])c([2H])c3oc4cccc([2H])c4c3c2c1[2H]]

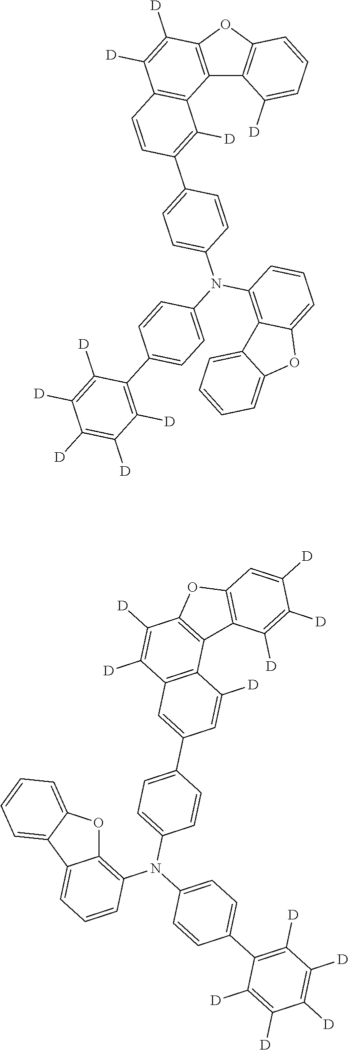 [SMILES: [2H]c1c([2H])c([2H])c(-c2ccc(N(c3ccc(-c4ccc5c([2H])c([2H])c6oc7cccc([2H])c7c6c5c4[2H])cc3)c3cccc4oc5ccccc5c34)cc2)c([2H])c1[2H] ; [2H]c1cc2oc3c([2H])c([2H])c4cc(-c5ccc(N(c6ccc(-c7c([2H])c([2H])c([2H])c([2H])c7[2H])cc6)c6cccc7c6oc6ccccc67)cc5)cc([2H])c4c3c2c([2H])c1[2H]]

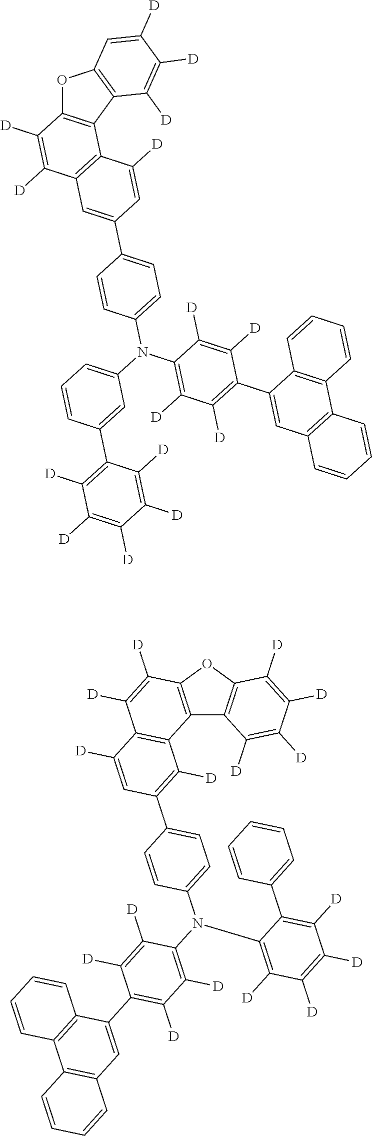 [SMILES: [2H]c1cc2oc3c([2H])c([2H])c4cc(-c5ccc(N(c6cccc(-c7c([2H])c([2H])c([2H])c([2H])c7[2H])c6)c6c([2H])c([2H])c(-c7cc8ccccc8c8ccccc78)c([2H])c6[2H])cc5)cc([2H])c4c3c2c([2H])c1[2H] ; [2H]c1c([2H])c([2H])c(N(c2ccc(-c3cc([2H])c4c([2H])c([2H])c5oc6c([2H])c([2H])c([2H])c([2H])c6c5c4c3[2H])cc2)c2c([2H])c([2H])c(-c3cc4ccccc4c4ccccc34)c([2H])c2[2H])c(-c2ccccc2)c1[2H]]

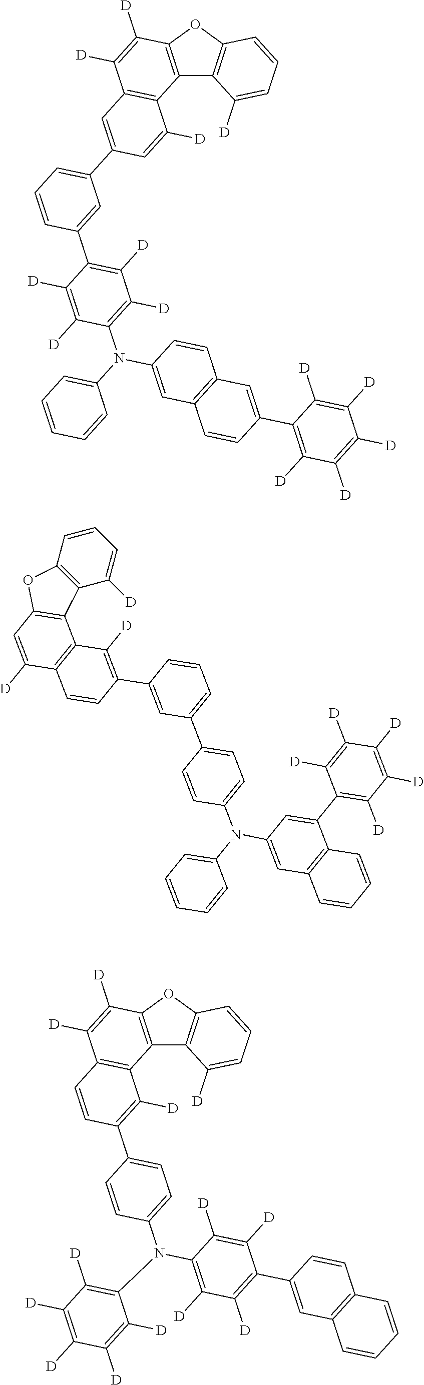 [SMILES: [2H]c1c([2H])c([2H])c(-c2ccc3cc(N(c4ccccc4)c4c([2H])c([2H])c(-c5cccc(-c6cc([2H])c7c(c6)c([2H])c([2H])c6oc8cccc([2H])c8c67)c5)c([2H])c4[2H])ccc3c2)c([2H])c1[2H] ; [2H]c1c([2H])c([2H])c(-c2cc(N(c3ccccc3)c3ccc(-c4cccc(-c5ccc6c([2H])cc7oc8cccc([2H])c8c7c6c5[2H])c4)cc3)cc3ccccc23)c([2H])c1[2H] ; [2H]c1c([2H])c([2H])c(N(c2ccc(-c3ccc4c([2H])c([2H])c5oc6cccc([2H])c6c5c4c3[2H])cc2)c2c([2H])c([2H])c(-c3ccc4ccccc4c3)c([2H])c2[2H])c([2H])c1[2H]]

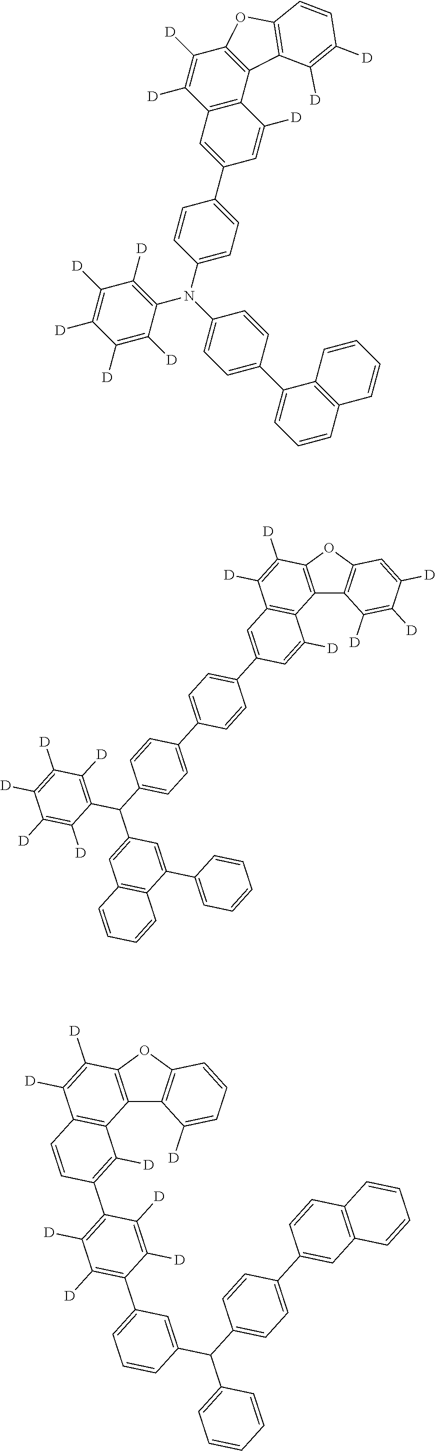 [SMILES: [2H]c1ccc2oc3c([2H])c([2H])c4cc(-c5ccc(N(c6ccc(-c7cccc8ccccc78)cc6)c6c([2H])c([2H])c([2H])c([2H])c6[2H])cc5)cc([2H])c4c3c2c1[2H] ; [2H]c1cc2oc3c([2H])c([2H])c4cc(-c5ccc(-c6ccc(C(c7cc(-c8ccccc8)c8ccccc8c7)c7c([2H])c([2H])c([2H])c([2H])c7[2H])cc6)cc5)cc([2H])c4c3c2c([2H])c1[2H] ; [2H]c1c([2H])c(-c2ccc3c([2H])c([2H])c4oc5cccc([2H])c5c4c3c2[2H])c([2H])c([2H])c1-c1cccc(C(c2ccccc2)c2ccc(-c3ccc4ccccc4c3)cc2)c1]

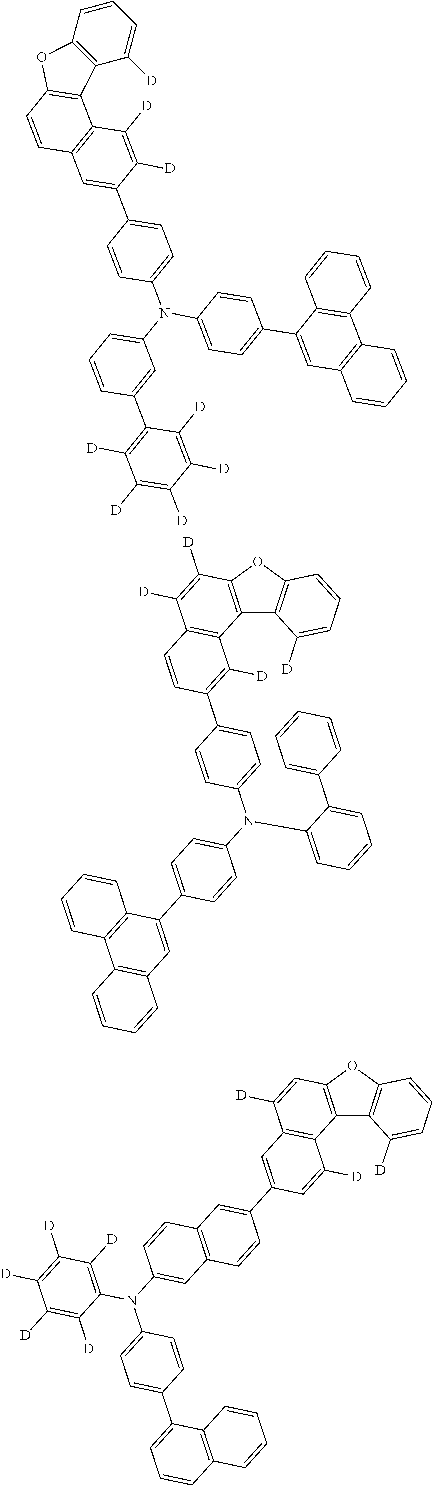

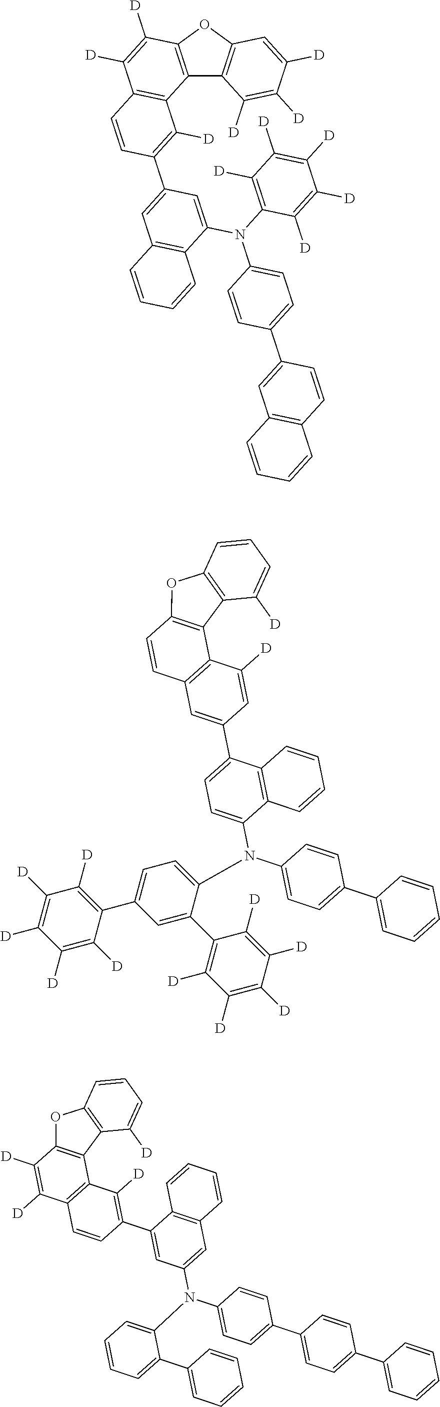 [SMILES: [2H]c1cc2oc3c([2H])c([2H])c4ccc(-c5cc(N(c6ccc(-c7ccc8ccccc8c7)cc6)c6c([2H])c([2H])c([2H])c([2H])c6[2H])c6ccccc6c5)c([2H])c4c3c2c([2H])c1[2H] ; [2H]c1c([2H])c([2H])c(-c2ccc(N(c3ccc(-c4ccccc4)cc3)c3ccc(-c4cc([2H])c5c(ccc6oc7cccc([2H])c7c65)c4)c4ccccc34)c(-c3c([2H])c([2H])c([2H])c([2H])c3[2H])c2)c([2H])c1[2H] ; [2H]c1c([2H])c2oc3cccc([2H])c3c2c2c([2H])c(-c3cc(N(c4ccc(-c5ccc(-c6ccccc6)cc5)cc4)c4ccccc4-c4ccccc4)cc4ccccc34)ccc12]

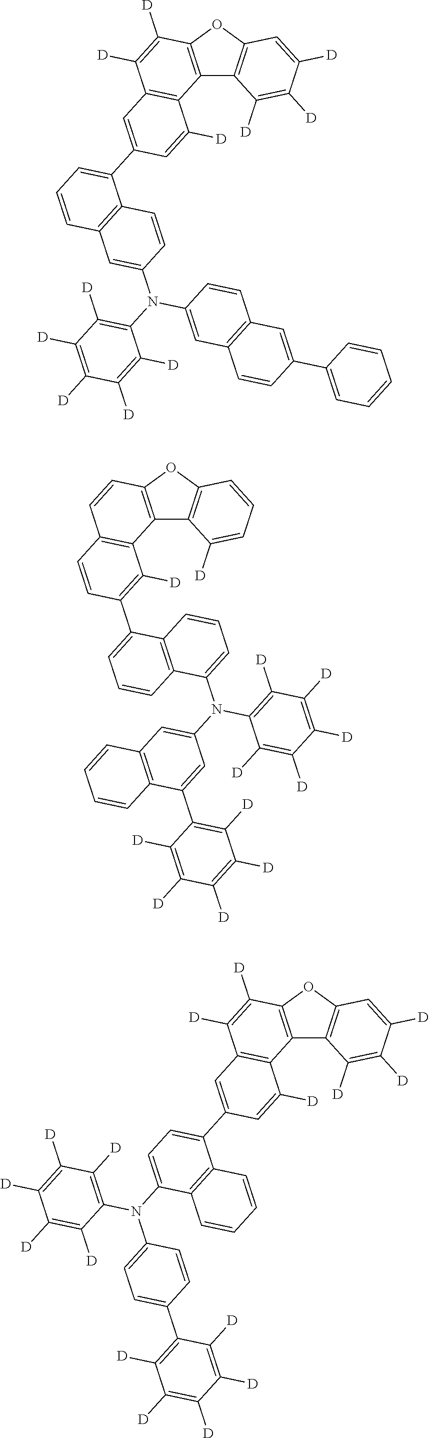 [SMILES: [2H]c1cc2oc3c([2H])c([2H])c4cc(-c5cccc6cc(N(c7ccc8cc(-c9ccccc9)ccc8c7)c7c([2H])c([2H])c([2H])c([2H])c7[2H])ccc56)cc([2H])c4c3c2c([2H])c1[2H] ; [2H]c1c([2H])c([2H])c(-c2cc(N(c3c([2H])c([2H])c([2H])c([2H])c3[2H])c3cccc4c(-c5ccc6ccc7oc8cccc([2H])c8c7c6c5[2H])cccc34)cc3ccccc23)c([2H])c1[2H] ; [2H]c1cc2oc3c([2H])c([2H])c4cc(-c5ccc(N(c6ccc(-c7c([2H])c([2H])c([2H])c([2H])c7[2H])cc6)c6c([2H])c([2H])c([2H])c([2H])c6[2H])c6ccccc56)cc([2H])c4c3c2c([2H])c1[2H]]

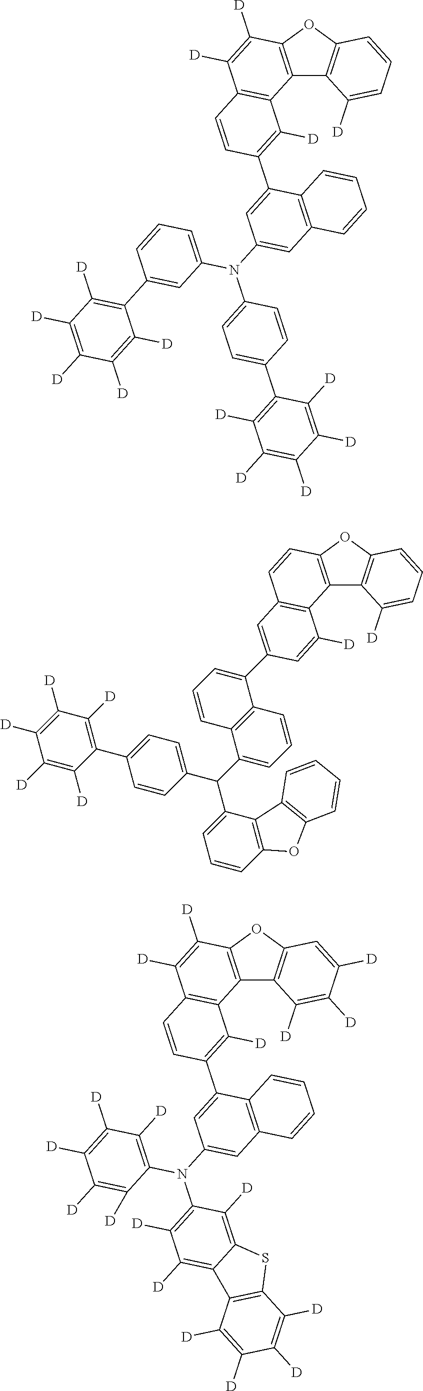 [SMILES: [2H]c1c([2H])c([2H])c(-c2ccc(N(c3cccc(-c4c([2H])c([2H])c([2H])c([2H])c4[2H])c3)c3cc(-c4ccc5c([2H])c([2H])c6oc7cccc([2H])c7c6c5c4[2H])c4ccccc4c3)cc2)c([2H])c1[2H] ; [2H]c1c([2H])c([2H])c(-c2ccc(C(c3cccc4c(-c5cc([2H])c6c(ccc7oc8cccc([2H])c8c76)c5)cccc34)c3cccc4oc5ccccc5c34)cc2)c([2H])c1[2H] ; [2H]c1cc2oc3c([2H])c([2H])c4ccc(-c5cc(N(c6c([2H])c([2H])c([2H])c([2H])c6[2H])c6c([2H])c([2H])c7c(sc8c([2H])c([2H])c([2H])c([2H])c87)c6[2H])cc6ccccc56)c([2H])c4c3c2c([2H])c1[2H]]

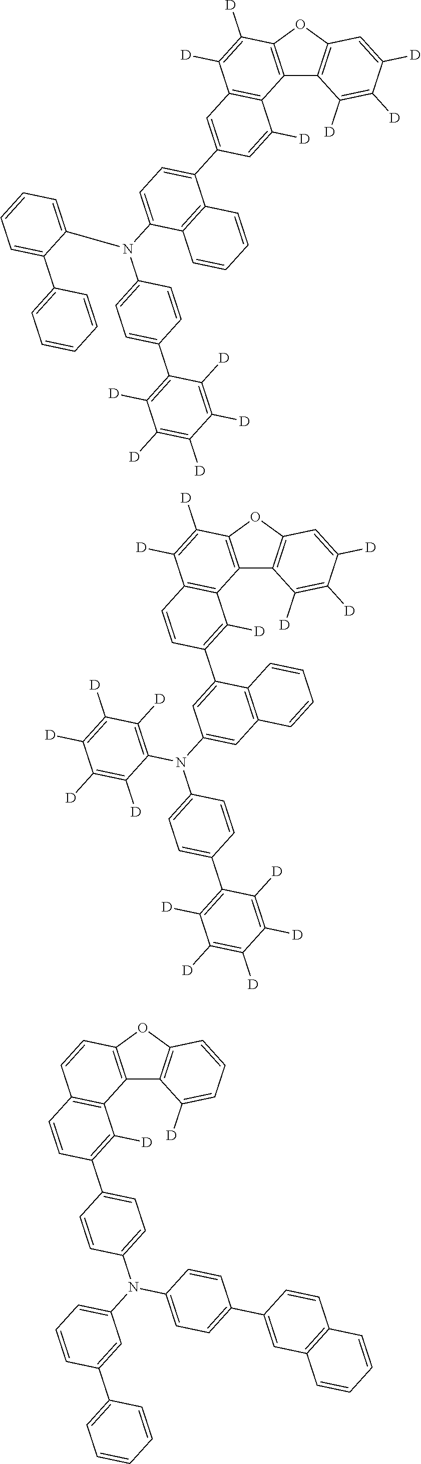

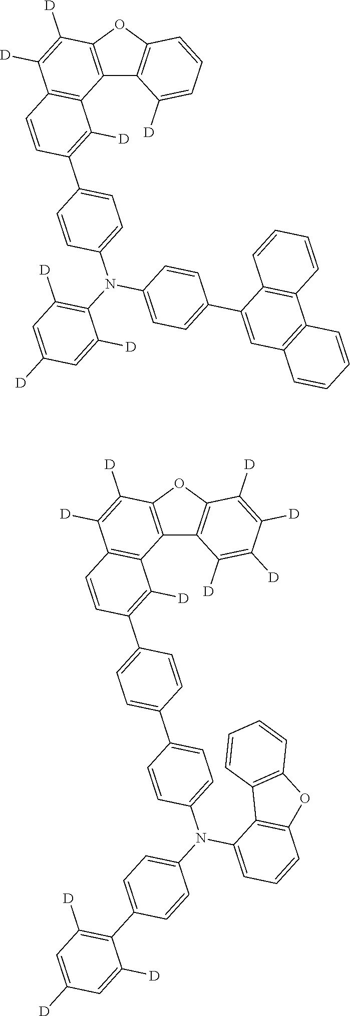 [SMILES: [2H]c1cc([2H])c(N(c2ccc(-c3ccc4c([2H])c([2H])c5oc6cccc([2H])c6c5c4c3[2H])cc2)c2ccc(-c3cc4ccccc4c4ccccc34)cc2)c([2H])c1 ; [2H]c1cc([2H])c(-c2ccc(N(c3ccc(-c4ccc(-c5ccc6c([2H])c([2H])c7oc8c([2H])c([2H])c([2H])c([2H])c8c7c6c5[2H])cc4)cc3)c3cccc4oc5ccccc5c34)cc2)c([2H])c1]

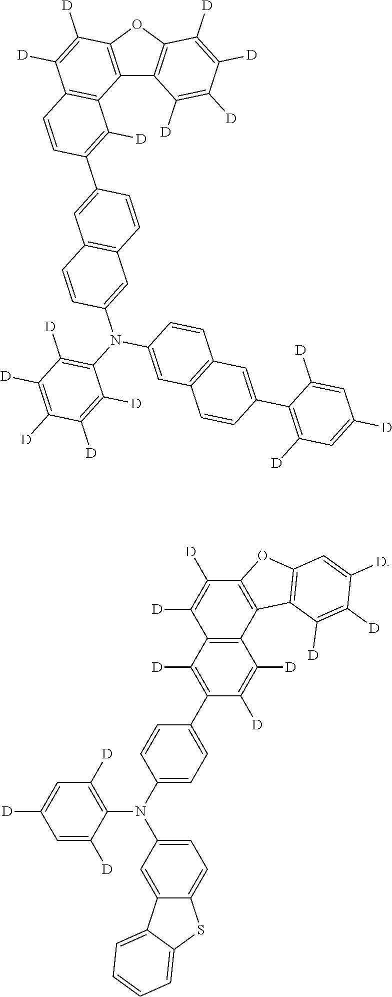

The compound of Chemical Formula 2, wherein R₇ is

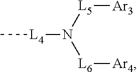

can be prepared by a preparation method as shown in the following Reaction Scheme 2 as an example, and the other remaining compounds cam be prepared in a similar manner.

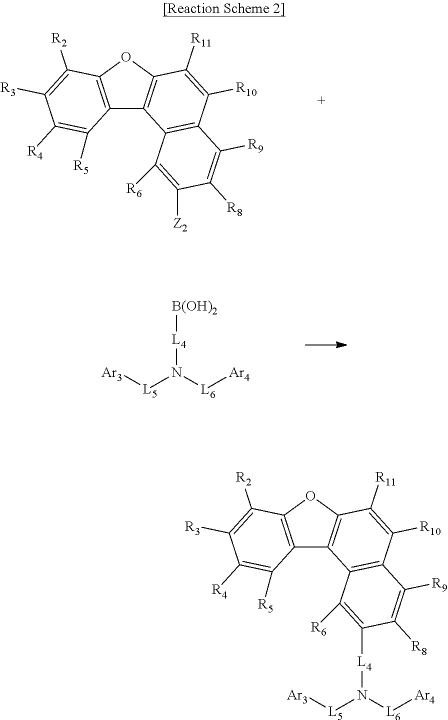

in Reaction Scheme 2, R₂ to R₁₁, Ar₃, Ar₄ and L₄ to L₆ are as defined in Chemical Formula 2, and Z₂ is halogen, preferably Z₂ is chloro or bromo.

Reaction Scheme 2 is a Suzuki coupling reaction, which is preferably carried out in the presence of a palladium catalyst and a base, and a reactive group for the Suzuki coupling reaction can be modified as known in the art. The above preparation method can be further embodied in Preparation Examples described hereinafter.

Preferably, in the light emitting layer, the weight ratio of the compound of Chemical Formula 1 and the compound of Chemical Formula 2 is 10:90 to 90:10, more preferably 20:80 to 80:20, 30:70 to 70:30 or 40:60 to 60:40.

Meanwhile, the light emitting layer can further include a dopant in addition to the host. The dopant material is not particularly limited as long as it is a material used for the organic light emitting device. As an example; an aromatic amine derivative, a styrylamine compound, a boron complex, a fluoranthene compound, a metal complex, and the like can be mentioned. Specific examples of the aromatic amine derivatives include substituted or unsubstituted fused aromatic ring derivatives having an arylamino group, examples thereof include pyrene, anthracene, chrysene, and periflanthene having the arylamino group, and the like. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, examples of the metal complex include an iridium complex, a platinum complex, and the like, but are not limited thereto.

In one example, the dopant material can be at least one selected from the group consisting of the following, without being limited thereto:

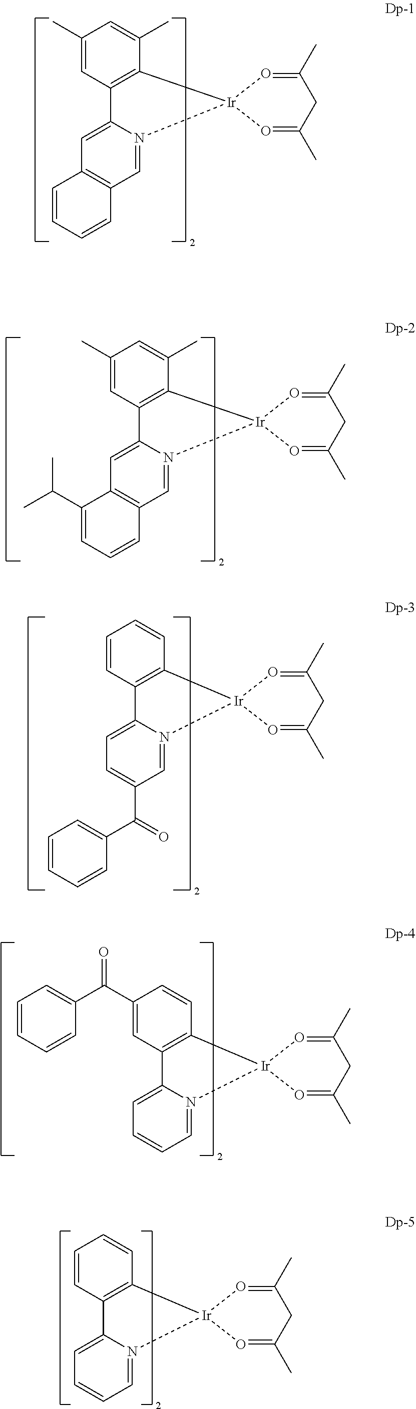

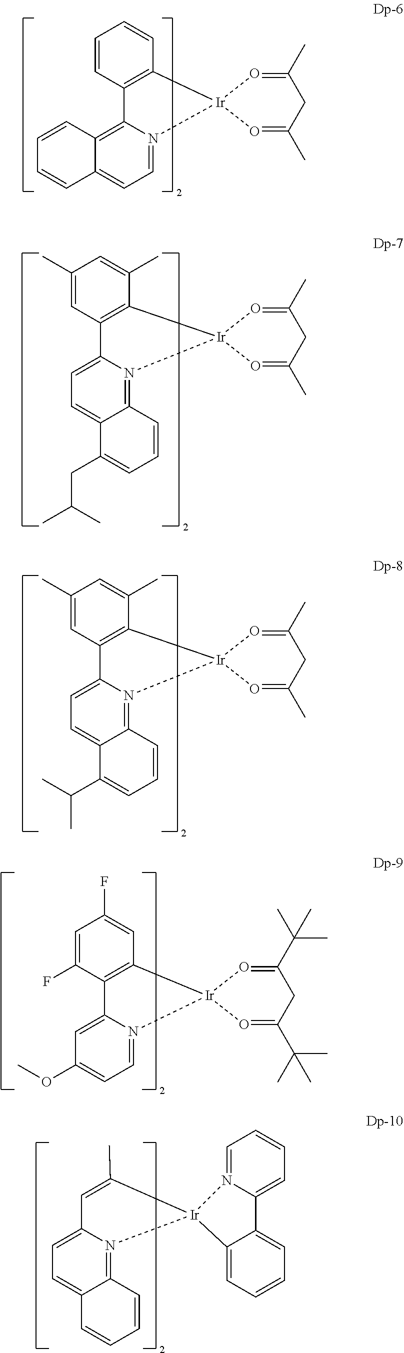

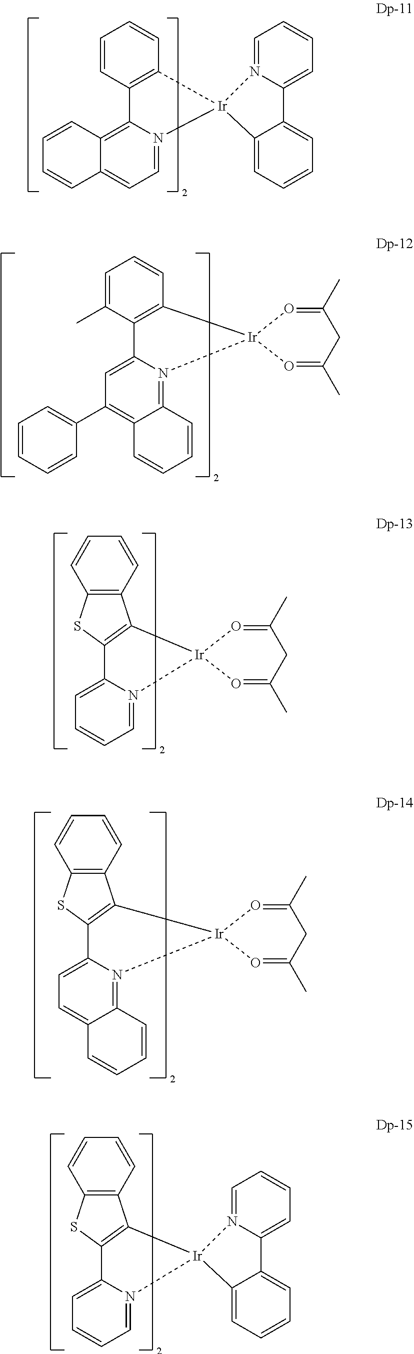

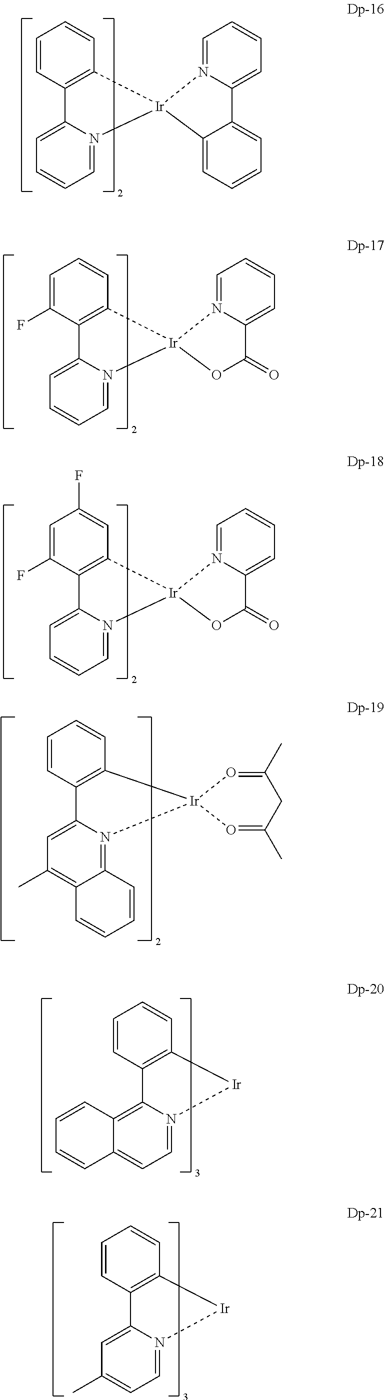

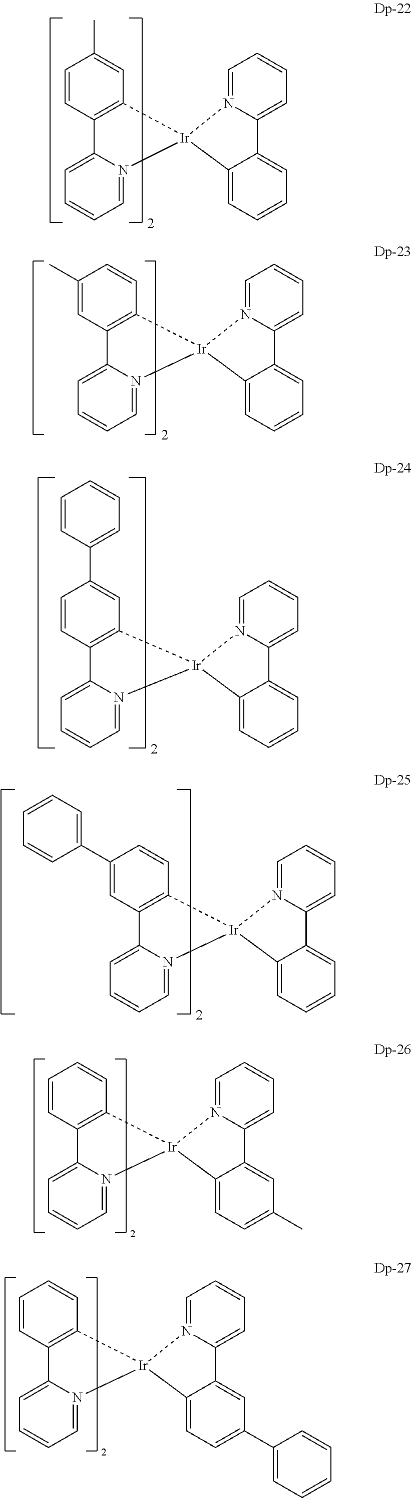

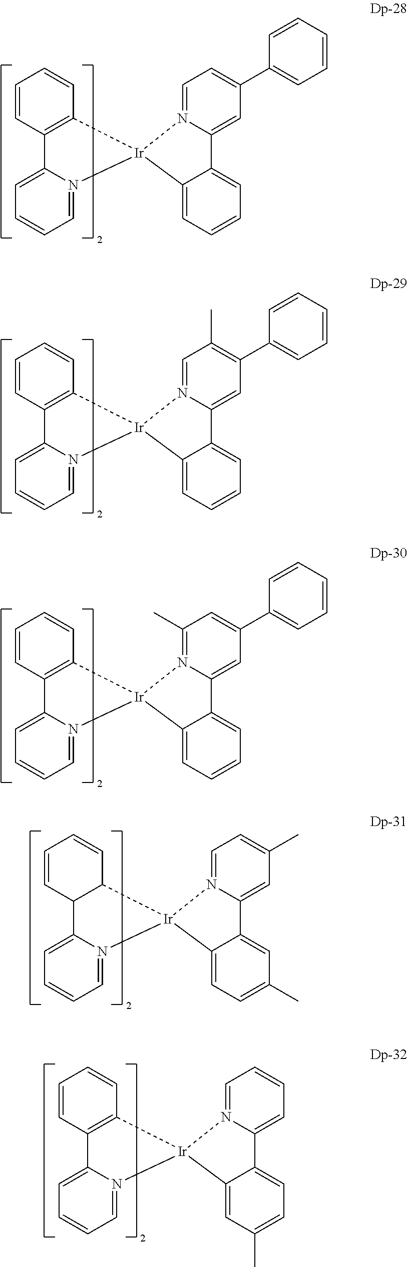

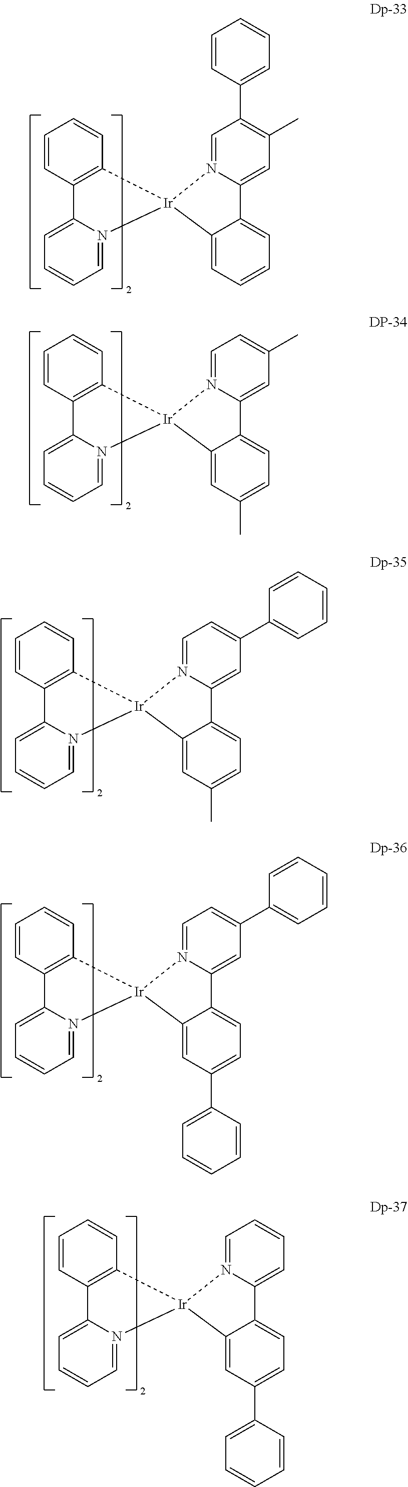

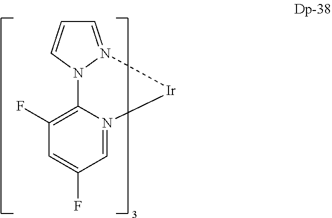

Hole Blocking Layer

The hole blocking layer is a layer provided between the electron transport layer and the light emitting layer in order to prevent the electrons injected in the anode from being transferred to the electron transport layer without being recombined in the light emitting layer, which can also be referred to as a hole inhibition layer. The hole blocking layer is preferably a material having high ionization energy.

Electron Transport Layer

The organic light emitting device according to the present disclosure can include an electron transport layer on the light emitting layer, if necessary.

The electron transport layer is a layer that receives the electrons from the electron injection layer formed on the cathode or the anode and transports the electrons to the light emitting layer, and that suppress the transfer of holes from the light emitting layer, and an electron transport material is suitably a material which can receive electrons well from a cathode and transfer the electrons to a light emitting layer, and has a large mobility for electrons.

Specific examples of the electron transport material include: an Al complex of 8-hydroxyquinoline, a complex including Alq₃, an organic radical compound, a hydroxyflavone-metal complex, and the like, but are not limited thereto. The electron transport layer can be used with any desired cathode material, as used according to a conventional technique. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

Electron Injection Layer

The organic light emitting device according to the present disclosure can further include an electron injection layer on the light emitting layer (or on the electron transport layer, if the electron transport layer exists).

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film.

Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxy-quinolinato)aluminum, tris(8-hydroxyquinolinato)gallium, bis(10-hydroxybenzo[h]quinolinato)beryllium, bis(10-hydroxybenzo[h]-quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

Meanwhile, in the present disclosure, the “electron injection and transport layer” is a layer that performs both the roles of the electron injection layer and the electron transport layer, and the materials that perform the roles of each layer can be used alone or in combination, without being limited thereto.

Organic Light Emitting Device

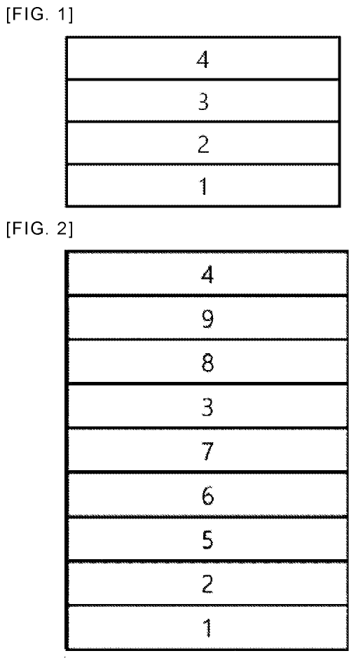

The structure of the organic light emitting device according to the present disclosure is illustrated in FIGS. 1 and 2. FIG. 1 shows an example of an organic light emitting device comprising a substrate 1, an anode 2, a light emitting layer 3, and a cathode 4. FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron injection and transport layer 9, and a cathode 4.

The organic light emitting device according to the present disclosure can be manufactured by sequentially stacking the above-described structures. In this case, the organic light emitting device can be manufactured by depositing a metal, metal oxides having conductivity, or an alloy thereof on the substrate by using a PVD (physical vapor deposition) method such as a sputtering method or an e-beam evaporation method to form the anode, forming the respective layers described above thereon, and then depositing a material that can be used as the cathode thereon. In addition to such a method, the organic light emitting device can be manufactured by sequentially depositing from the cathode material to the anode material on a substrate in the reverse order of the above-mentioned configuration (WO 2003/012890). Further, the light emitting layer can be formed by subjecting hosts and dopants to a vacuum deposition method and a solution coating method. Herein, the solution coating method means a spin coating, a dip coating, a doctor blading, an inkjet printing, a screen printing, a spray method, a roll coating, or the like, but is not limited thereto.

Meanwhile, the organic light emitting device according to the present disclosure can be a bottom emission device, a top emission device, or a double-sided light emitting device, and particularly, can be a bottom emission device that requires relatively high luminous efficiency.

Hereinafter, preferred examples are presented to assist in the understanding of the present disclosure. However, the following examples are only provided for a better understanding of the present disclosure, and is not intended to limit the content of the present disclosure.

### Synthesis Example 1-1

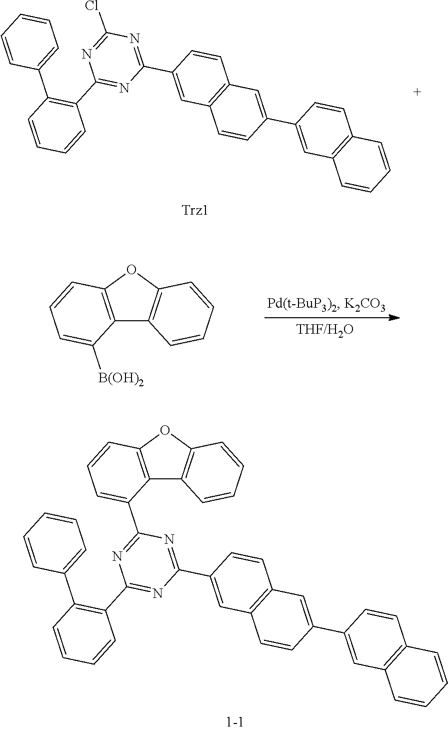 [SMILES: Clc1nc(-c2ccc3cc(-c4ccc5ccccc5c4)ccc3c2)nc(-c2ccccc2-c2ccccc2)n1 ; OB(O)c1cccc2oc3ccccc3c12 ; c1ccc(-c2ccccc2-c2nc(-c3ccc4cc(-c5ccc6ccccc6c5)ccc4c3)nc(-c3cccc4oc5ccccc5c34)n2)cc1]

Compound Trz1 (15 g, 28.8 mmol) and dibenzo[b,d]furan-1-ylboronic acid (6.4 g, 30.3 mmol) were added to 300 ml of THF, and the mixture was stirred and refluxed. Then, potassium carbonate (12 g, 86.5 mmol) was dissolved in 36 ml of water and added thereto, and the mixture was sufficiently stirred and then bis(tri-tert-butylphosphine)palladium(0) (0.1 g, 0.3 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.2 g of Compound 1-1. (Yield: 65%, MS: [M+H]⁺=652)

### Synthesis Example 1-2

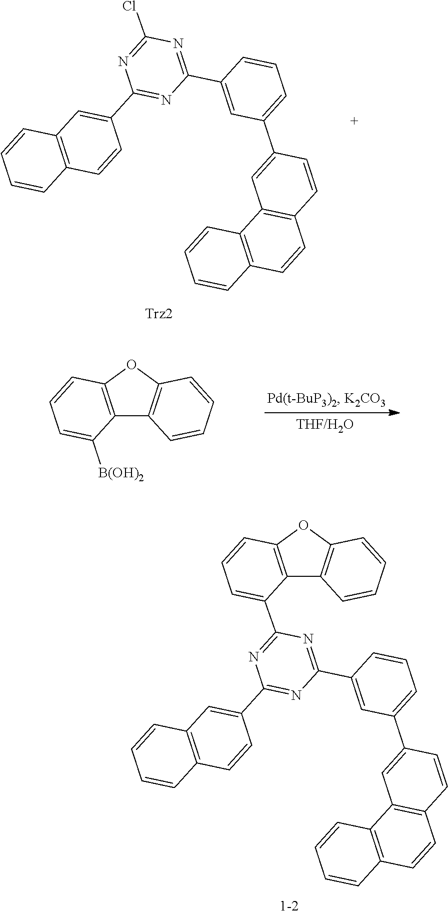 [SMILES: Clc1nc(-c2cccc(-c3ccc4ccc5ccccc5c4c3)c2)nc(-c2ccc3ccccc3c2)n1 ; OB(O)c1cccc2oc3ccccc3c12 ; c1cc(-c2ccc3ccc4ccccc4c3c2)cc(-c2nc(-c3ccc4ccccc4c3)nc(-c3cccc4oc5ccccc5c34)n2)c1]

Compound Trz2 (15 g, 30.4 mmol) and dibenzo[b,d]furan-1-ylboronic acid (6.8 g, 31.9 mmol) were added to 300 ml of THF, and the mixture was stirred and refluxed. Then, potassium carbonate (12.6 g, 91.1 mmol) was dissolved in 36 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14 g of Compound 1-2. (Yield: 74%, MS: [M+H]⁺=626)

### Synthesis Example 1-3

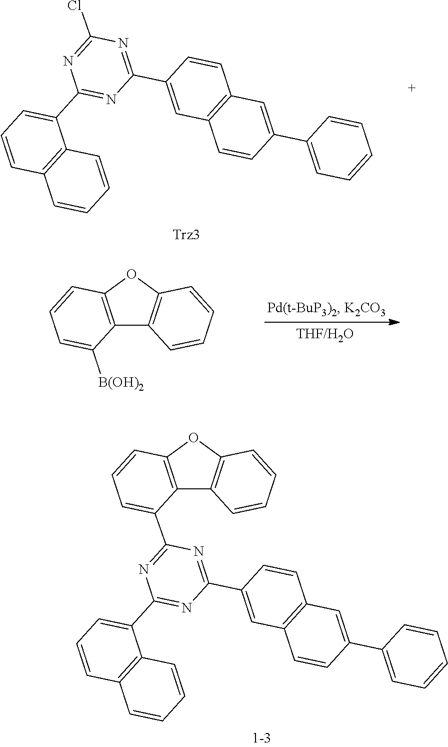 [SMILES: Clc1nc(-c2ccc3cc(-c4ccccc4)ccc3c2)nc(-c2cccc3ccccc23)n1 ; OB(O)c1cccc2oc3ccccc3c12 ; c1ccc(-c2ccc3cc(-c4nc(-c5cccc6ccccc56)nc(-c5cccc6oc7ccccc7c56)n4)ccc3c2)cc1]

Compound Trz3 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.4 g of Compound 1-3. (Yield: 69%, MS: [M+H]⁺=576)

### Synthesis Example 1-4

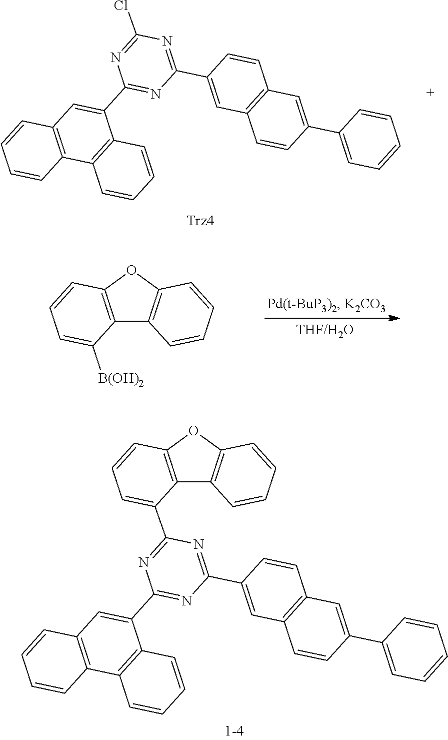 [SMILES: Clc1nc(-c2ccc3cc(-c4ccccc4)ccc3c2)nc(-c2cc3ccccc3c3ccccc23)n1 ; OB(O)c1cccc2oc3ccccc3c12 ; c1ccc(-c2ccc3cc(-c4nc(-c5cc6ccccc6c6ccccc56)nc(-c5cccc6oc7ccccc7c56)n4)ccc3c2)cc1]

Compound Trz4 (15 g, 30.4 mmol) and dibenzo[b,d]furan-1-ylboronic acid (6.8 g, 31.9 mmol) were added to 300 ml of THF, and the mixture was stirred and refluxed. Then, potassium carbonate (12.6 g, 91.1 mmol) was dissolved in 38 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.3 g of Compound 1-4. (Yield: 70%, MS: [M+H]⁺=626)

### Synthesis Example 1-5

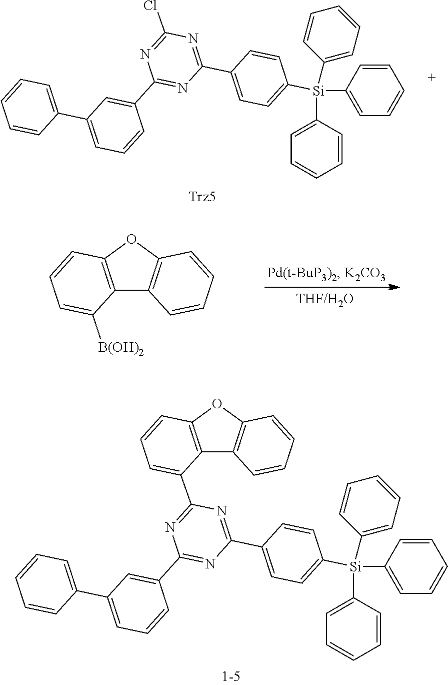 [SMILES: Clc1nc(-c2ccc([Si](c3ccccc3)(c3ccccc3)c3ccccc3)cc2)nc(-c2cccc(-c3ccccc3)c2)n1 ; OB(O)c1cccc2oc3ccccc3c12 ; c1ccc(-c2cccc(-c3nc(-c4ccc([Si](c5ccccc5)(c5ccccc5)c5ccccc5)cc4)nc(-c4cccc5oc6ccccc6c45)n3)c2)cc1]

Compound Trz5 (15 g, 24.9 mmol) and dibenzo[b,d]furan-1-ylboronic acid (5.5 g, 26.2 mmol) were added to 300 ml of THF, and the mixture was stirred and refluxed. Then, potassium carbonate (10.3 g, 74.7 mmol) was dissolved in 31 ml of water and added thereto, and the mixture was sufficiently stirred and then bis(tri-tert-butylphosphine)palladium(0) (0.1 g, 0.2 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.6 g of Compound 1-5. (Yield: 69%, MS: [M+H]⁺=734)

### Synthesis Example 1-6

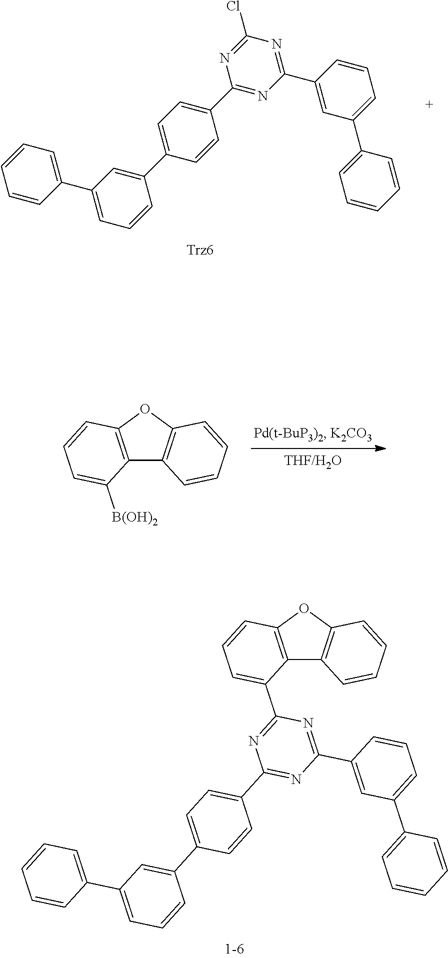 [SMILES: Clc1nc(-c2ccc(-c3cccc(-c4ccccc4)c3)cc2)nc(-c2cccc(-c3ccccc3)c2)n1 ; OB(O)c1cccc2oc3ccccc3c12 ; c1ccc(-c2cccc(-c3ccc(-c4nc(-c5cccc(-c6ccccc6)c5)nc(-c5cccc6oc7ccccc7c56)n4)cc3)c2)cc1]

Compound Trz6 (15 g, 30.2 mmol) and dibenzo[b,d]furan-1-ylboronic acid (6.7 g, 31.8 mmol) were added to 300 ml of THF, and the mixture was stirred and refluxed. Then, potassium carbonate (12.5 g, 90.7 mmol) was dissolved in 38 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.5 g of Compound 1-6. (Yield: 66%, MS: [M+H]⁺=629)

### Synthesis Example 1-7

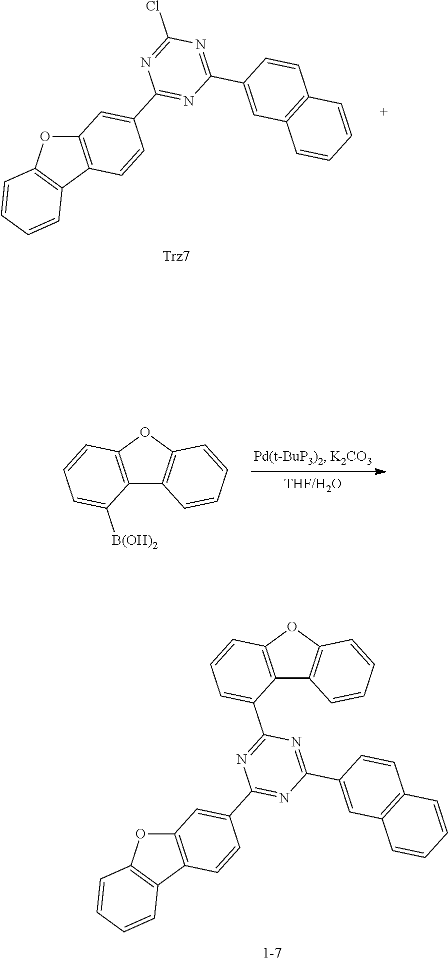 [SMILES: Clc1nc(-c2ccc3ccccc3c2)nc(-c2ccc3c(c2)oc2ccccc23)n1 ; OB(O)c1cccc2oc3ccccc3c12 ; c1ccc2cc(-c3nc(-c4ccc5c(c4)oc4ccccc45)nc(-c4cccc5oc6ccccc6c45)n3)ccc2c1]

Compound Trz7 (15 g, 36.8 mmol) and dibenzo[b,d]furan-1-ylboronic acid (8.2 g, 38.6 mmol) were added to 300 ml of THF, and the mixture was stirred and refluxed. Then, potassium carbonate (15.2 g, 110.3 mmol) was dissolved in 46 ml of water and added thereto, and the mixture was sufficiently stirred and then bis(tri-tert-butylphosphine)palladium(0) (0.2 g, 0.4 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.9 g of Compound 1-7. (Yield: 75%, MS: [M+H]⁺=540)

### Synthesis Example 1-8

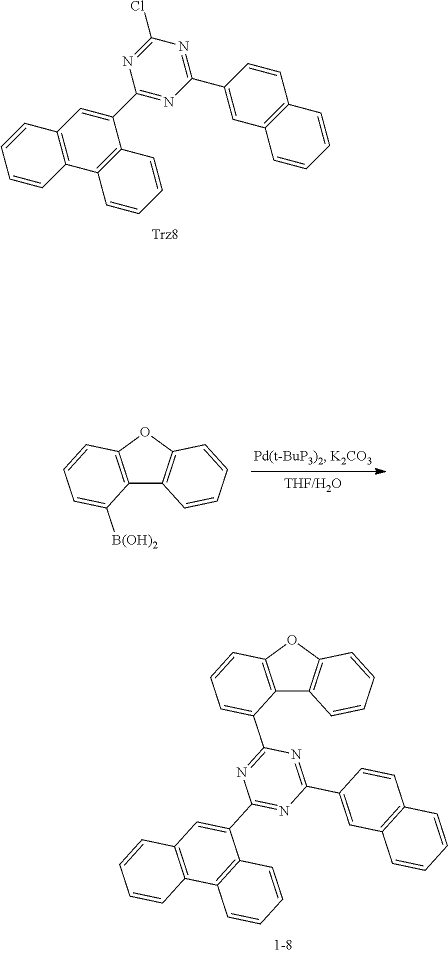 [SMILES: Clc1nc(-c2ccc3ccccc3c2)nc(-c2cc3ccccc3c3ccccc23)n1 ; OB(O)c1cccc2oc3ccccc3c12 ; c1ccc2cc(-c3nc(-c4cc5ccccc5c5ccccc45)nc(-c4cccc5oc6ccccc6c45)n3)ccc2c1]

Compound Trz8 (15 g, 35.9 mmol) and dibenzo[b,d]furan-1-ylboronic acid (8 g, 37.7 mmol) were added to 300 ml of THF, and the mixture was stirred and refluxed. Then, potassium carbonate (14.9 g, 107.7 mmol) was dissolved in 45 ml of water and added thereto, and the mixture was sufficiently stirred and then bis(tri-tert-butylphosphine)palladium(0) (0.2 g, 0.4 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.8 g of Compound 1-8. (Yield: 70%, MS: [M+H]⁺=550)

### Synthesis Example 1-9

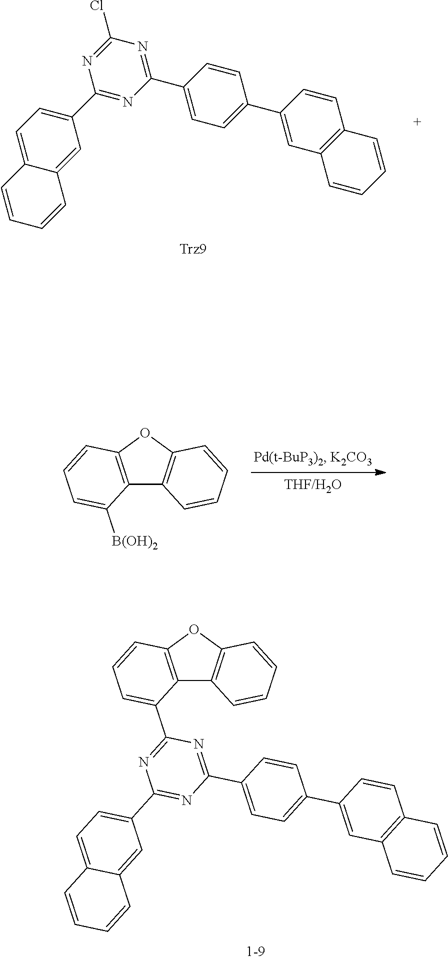 [SMILES: Clc1nc(-c2ccc(-c3ccc4ccccc4c3)cc2)nc(-c2ccc3ccccc3c2)n1 ; OB(O)c1cccc2oc3ccccc3c12 ; c1ccc2cc(-c3ccc(-c4nc(-c5ccc6ccccc6c5)nc(-c5cccc6oc7ccccc7c56)n4)cc3)ccc2c1]

Compound Trz9 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.6 g of Compound 1-9. (Yield: 70%, MS: [M+H]⁺=576)

### Synthesis Example 1-10

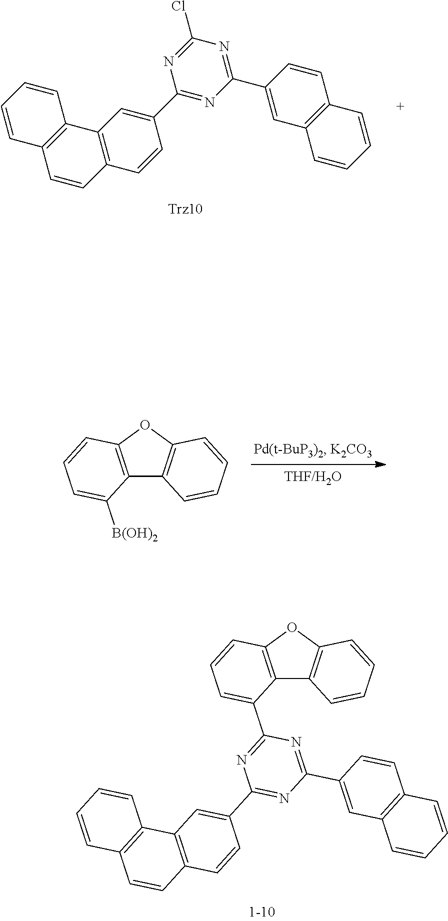 [SMILES: Clc1nc(-c2ccc3ccccc3c2)nc(-c2ccc3ccc4ccccc4c3c2)n1 ; OB(O)c1cccc2oc3ccccc3c12 ; c1ccc2cc(-c3nc(-c4ccc5ccc6ccccc6c5c4)nc(-c4cccc5oc6ccccc6c45)n3)ccc2c1]

Compound Trz10 (15 g, 35.9 mmol) and dibenzo[b,d]furan-1-ylboronic acid (8 g, 37.7 mmol) were added to 300 ml of THF, and the mixture was stirred and refluxed. Then, potassium carbonate (14.9 g, 107.7 mmol) was dissolved in 45 ml of water and added thereto, and the mixture was sufficiently stirred and then bis(tri-tert-butylphosphine)palladium(0) (0.2 g, 0.4 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.8 g of Compound 1-10. (Yield: 70%, MS: [M+H]⁺=550)

### Synthesis Example 1-11

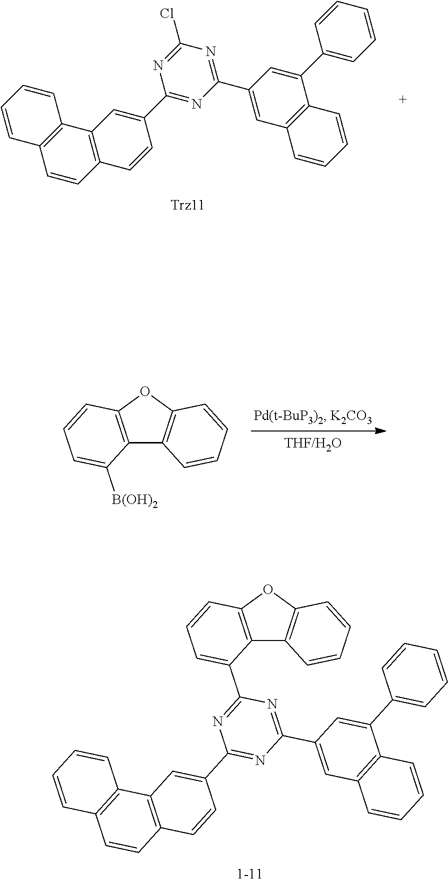 [SMILES: Clc1nc(-c2cc(-c3ccccc3)c3ccccc3c2)nc(-c2ccc3ccc4ccccc4c3c2)n1 ; OB(O)c1cccc2oc3ccccc3c12 ; c1ccc(-c2cc(-c3nc(-c4ccc5ccc6ccccc6c5c4)nc(-c4cccc5oc6ccccc6c45)n3)cc3ccccc23)cc1]

Compound Trz11 (15 g, 30.4 mmol) and dibenzo[b,d]furan-1-ylboronic acid (6.8 g, 31.9 mmol) were added to 300 ml of THF, and the mixture was stirred and refluxed. Then, potassium carbonate (12.6 g, 91.1 mmol) was dissolved in 38 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.7 g of Compound 1-11. (Yield: 72%, MS: [M+H]⁺=626)

### Synthesis Example 1-12

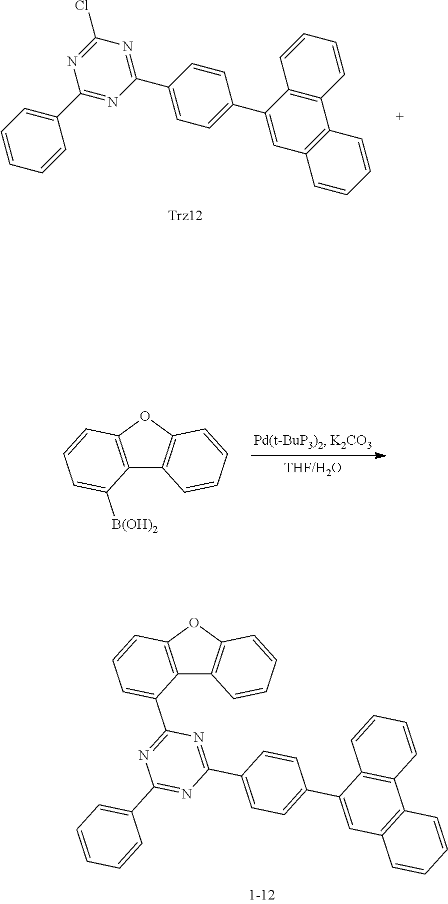 [SMILES: Clc1nc(-c2ccccc2)nc(-c2ccc(-c3cc4ccccc4c4ccccc34)cc2)n1 ; OB(O)c1cccc2oc3ccccc3c12 ; c1ccc(-c2nc(-c3ccc(-c4cc5ccccc5c5ccccc45)cc3)nc(-c3cccc4oc5ccccc5c34)n2)cc1]

Compound Trz12 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.2 g of Compound 1-12. (Yield: 73%, MS: [M+H]⁺=576)

### Synthesis Example 1-13

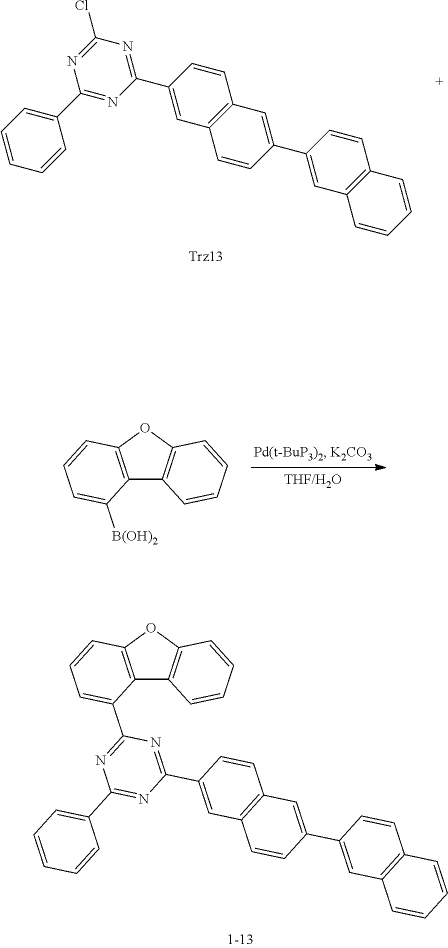 [SMILES: Clc1nc(-c2ccccc2)nc(-c2ccc3cc(-c4ccc5ccccc5c4)ccc3c2)n1 ; OB(O)c1cccc2oc3ccccc3c12 ; c1ccc(-c2nc(-c3ccc4cc(-c5ccc6ccccc6c5)ccc4c3)nc(-c3cccc4oc5ccccc5c34)n2)cc1]

Compound Trz13 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.4 g of Compound 1-13. (Yield: 69%, MS: [M+H]⁺=576)

### Synthesis Example 1-14

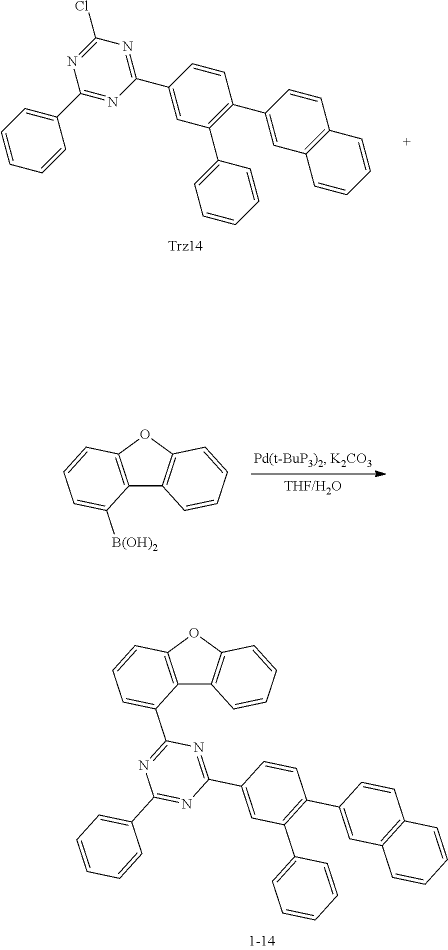 [SMILES: Clc1nc(-c2ccccc2)nc(-c2ccc(-c3ccc4ccccc4c3)c(-c3ccccc3)c2)n1 ; OB(O)c1cccc2oc3ccccc3c12 ; c1ccc(-c2nc(-c3ccc(-c4ccc5ccccc5c4)c(-c4ccccc4)c3)nc(-c3cccc4oc5ccccc5c34)n2)cc1]

Compound Trz14 (15 g, 31.9 mmol) and dibenzo[b,d]furan-1-ylboronic acid (7.1 g, 33.5 mmol) were added to 300 ml of THF, and the mixture was stirred and refluxed. Then, potassium carbonate (13.2 g, 95.8 mmol) was dissolved in 40 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.2 g of Compound 1-14. (Yield: 74%, MS: [M+H]⁺=602)

### Synthesis Example 1-15

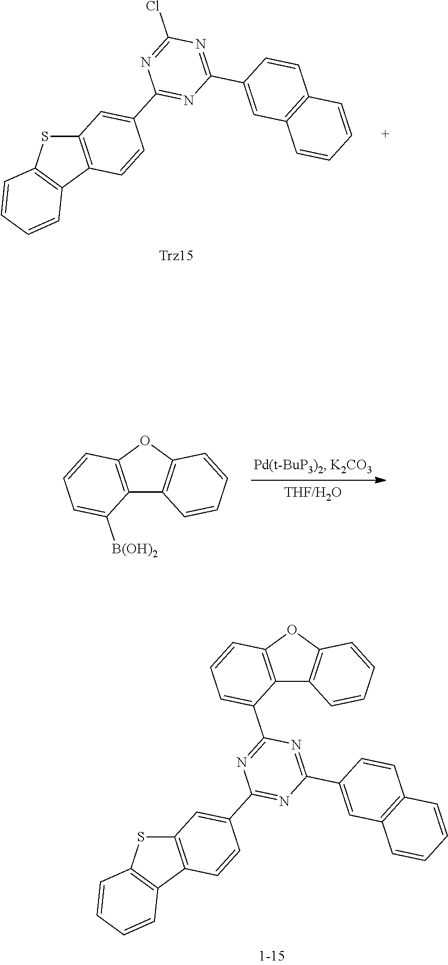 [SMILES: Clc1nc(-c2ccc3ccccc3c2)nc(-c2ccc3c(c2)sc2ccccc23)n1 ; OB(O)c1cccc2oc3ccccc3c12 ; c1ccc2cc(-c3nc(-c4ccc5c(c4)sc4ccccc45)nc(-c4cccc5oc6ccccc6c45)n3)ccc2c1]

Compound Trz15 (15 g, 35.4 mmol) and dibenzo[b,d]furan-1-ylboronic acid (7.9 g, 37.2 mmol) were added to 300 ml of THF, and the mixture was stirred and refluxed. Then, potassium carbonate (14.7 g, 106.2 mmol) was dissolved in 44 ml of water and added thereto, and the mixture was sufficiently stirred and then bis(tri-tert-butylphosphine)palladium(0) (0.2 g, 0.4 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.3 g of Compound 1-15. (Yield: 73%, MS: [M+H]⁺=556)

### Synthesis Example 1-16

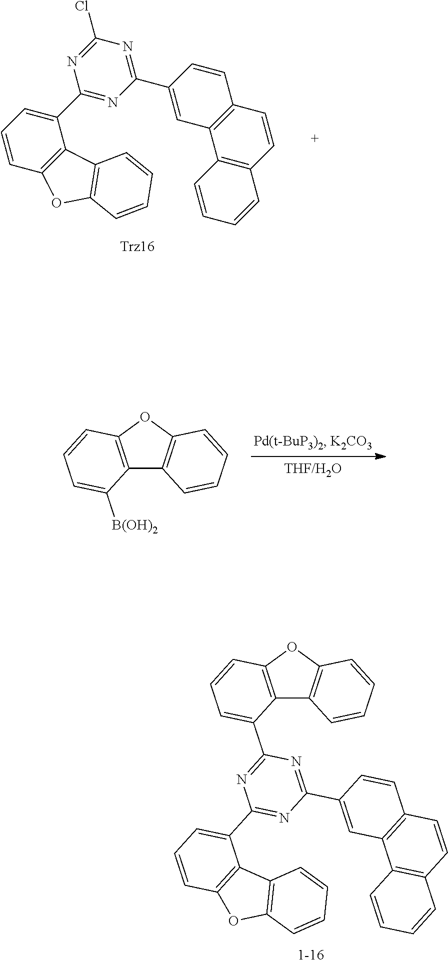 [SMILES: Clc1nc(-c2ccc3ccc4ccccc4c3c2)nc(-c2cccc3oc4ccccc4c23)n1 ; OB(O)c1cccc2oc3ccccc3c12 ; c1ccc2c(c1)ccc1ccc(-c3nc(-c4cccc5oc6ccccc6c45)nc(-c4cccc5oc6ccccc6c45)n3)cc12]

Compound Trz16 (15 g, 32.8 mmol) and dibenzo[b,d]furan-1-ylboronic acid (7.3 g, 34.4 mmol) were added to 300 ml of THF, and the mixture was stirred and refluxed. Then, potassium carbonate (13.6 g, 98.3 mmol) was dissolved in 41 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.3 g of Compound 1-16. (Yield: 74%, MS: [M+H]⁺=590)

### Synthesis Example 1-17

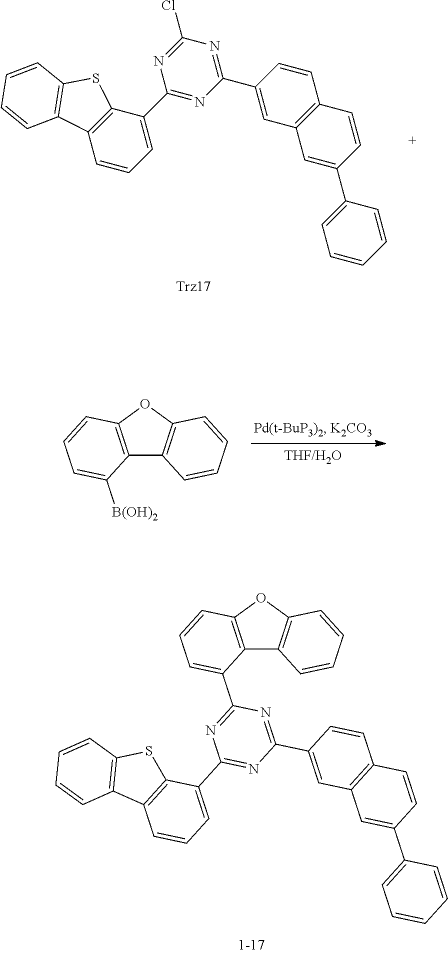 [SMILES: Clc1nc(-c2ccc3ccc(-c4ccccc4)cc3c2)nc(-c2cccc3c2sc2ccccc23)n1 ; OB(O)c1cccc2oc3ccccc3c12 ; c1ccc(-c2ccc3ccc(-c4nc(-c5cccc6c5sc5ccccc56)nc(-c5cccc6oc7ccccc7c56)n4)cc3c2)cc1]

Compound Trz17 (15 g, 30 mmol) and dibenzo[b,d]furan-1-ylboronic acid (6.7 g, 31.5 mmol) were added to 300 ml of THF, and the mixture was stirred and refluxed. Then, potassium carbonate (12.4 g, 90 mmol) was dissolved in 37 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14 g of Compound 1-17. (Yield: 74%, MS: [M+H]⁺=632)

### Synthesis Example 1-18

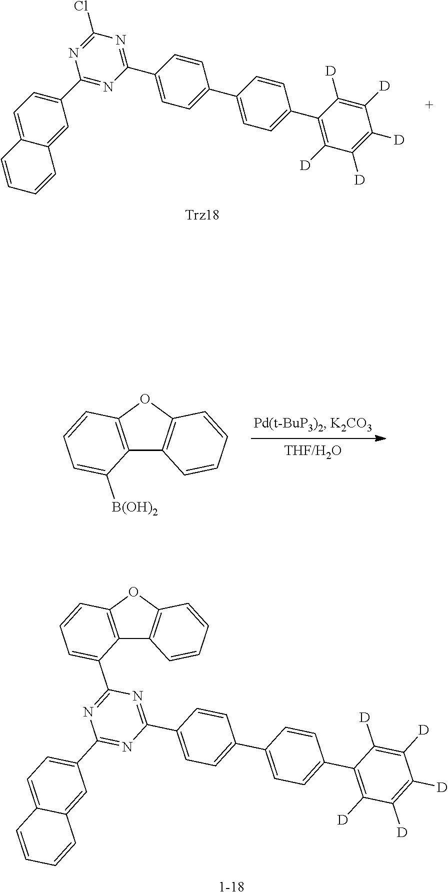 [SMILES: [2H]c1c([2H])c([2H])c(-c2ccc(-c3ccc(-c4nc(Cl)nc(-c5ccc6ccccc6c5)n4)cc3)cc2)c([2H])c1[2H] ; OB(O)c1cccc2oc3ccccc3c12 ; [2H]c1c([2H])c([2H])c(-c2ccc(-c3ccc(-c4nc(-c5ccc6ccccc6c5)nc(-c5cccc6oc7ccccc7c56)n4)cc3)cc2)c([2H])c1[2H]]

Compound Trz17 (15 g, 31.6 mmol) and dibenzo[b,d]furan-1-ylboronic acid (7 g, 33.2 mmol) were added to 300 ml of THF, and the mixture was stirred and refluxed. Then, potassium carbonate (13.1 g, 94.7 mmol) was dissolved in 39 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.2 g of Compound 1-18. (Yield: 74%, MS: [M+H]⁺=607)

### Synthesis Example 1-19

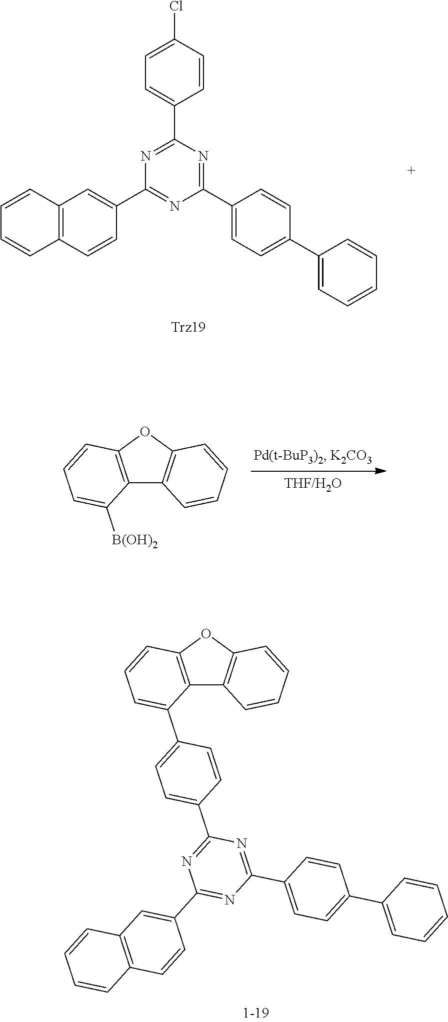 [SMILES: Clc1ccc(-c2nc(-c3ccc(-c4ccccc4)cc3)nc(-c3ccc4ccccc4c3)n2)cc1 ; OB(O)c1cccc2oc3ccccc3c12 ; c1ccc(-c2ccc(-c3nc(-c4ccc(-c5cccc6oc7ccccc7c56)cc4)nc(-c4ccc5ccccc5c4)n3)cc2)cc1]

Compound Trz19 (15 g, 31.9 mmol) and dibenzo[b,d]furan-1-ylboronic acid (7.1 g, 33.5 mmol) were added to 300 ml of THF, and the mixture was stirred and refluxed. Then, potassium carbonate (13.2 g, 95.8 mmol) was dissolved in 40 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.7 g of Compound 1-19. (Yield: 66%, MS: [M+H]⁺=602)

### Synthesis Example 1-20

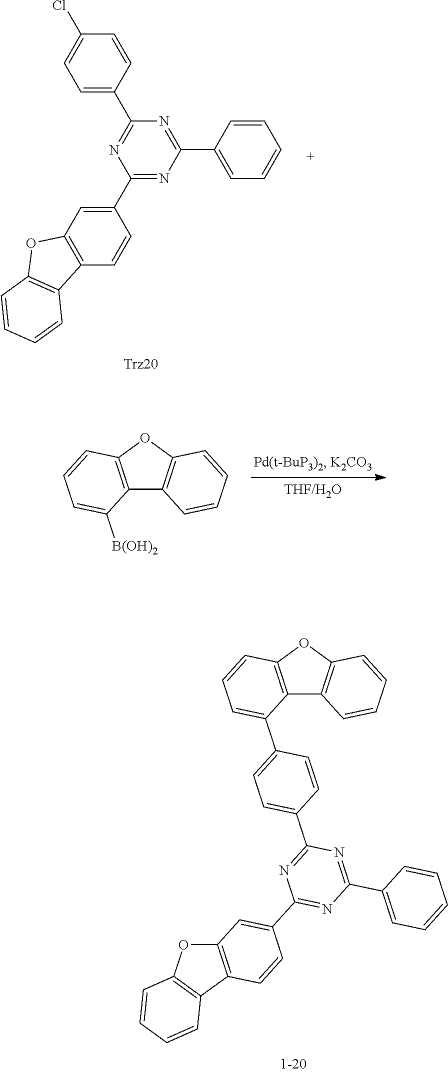 [SMILES: Clc1ccc(-c2nc(-c3ccccc3)nc(-c3ccc4c(c3)oc3ccccc34)n2)cc1 ; OB(O)c1cccc2oc3ccccc3c12 ; c1ccc(-c2nc(-c3ccc(-c4cccc5oc6ccccc6c45)cc3)nc(-c3ccc4c(c3)oc3ccccc34)n2)cc1]

Compound Trz20 (15 g, 34.6 mmol) and dibenzo[b,d]furan-1-ylboronic acid (7.7 g, 36.3 mmol) were added to 300 ml of THF, and the mixture was stirred and refluxed. Then, potassium carbonate (14.3 g, 103.7 mmol) was dissolved in 43 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.9 g of Compound 1-20. (Yield: 71%, MS: [M+H]⁺=566)

### Synthesis Example 1-21

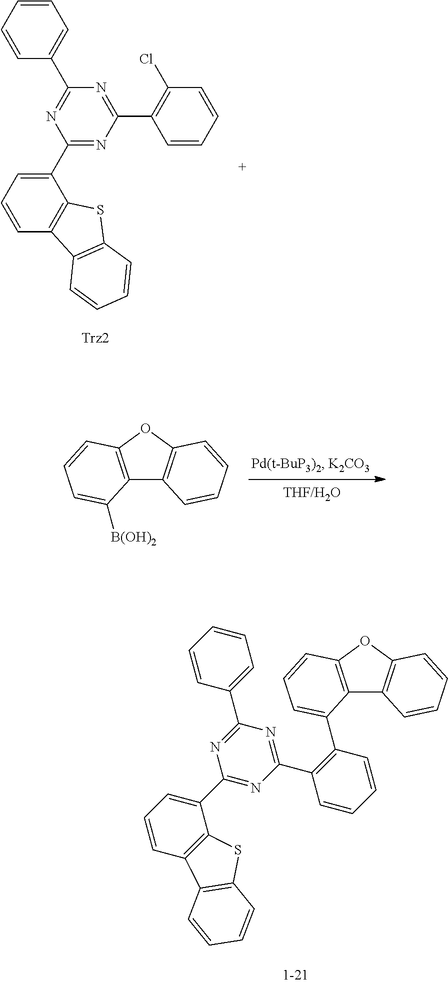 [SMILES: Clc1ccccc1-c1nc(-c2ccccc2)nc(-c2cccc3c2sc2ccccc23)n1 ; OB(O)c1cccc2oc3ccccc3c12 ; c1ccc(-c2nc(-c3ccccc3-c3cccc4oc5ccccc5c34)nc(-c3cccc4c3sc3ccccc34)n2)cc1]

Compound Trz21 (15 g, 33.3 mmol) and dibenzo[b,d]furan-1-ylboronic acid (7.4 g, 35 mmol) were added to 300 ml of THF, and the mixture was stirred and refluxed. Then, potassium carbonate (13.8 g, 100 mmol) was dissolved in 41 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.9 g of Compound 1-21. (Yield: 72%, MS: [M+H]⁺=582)

### Synthesis Example 1-22

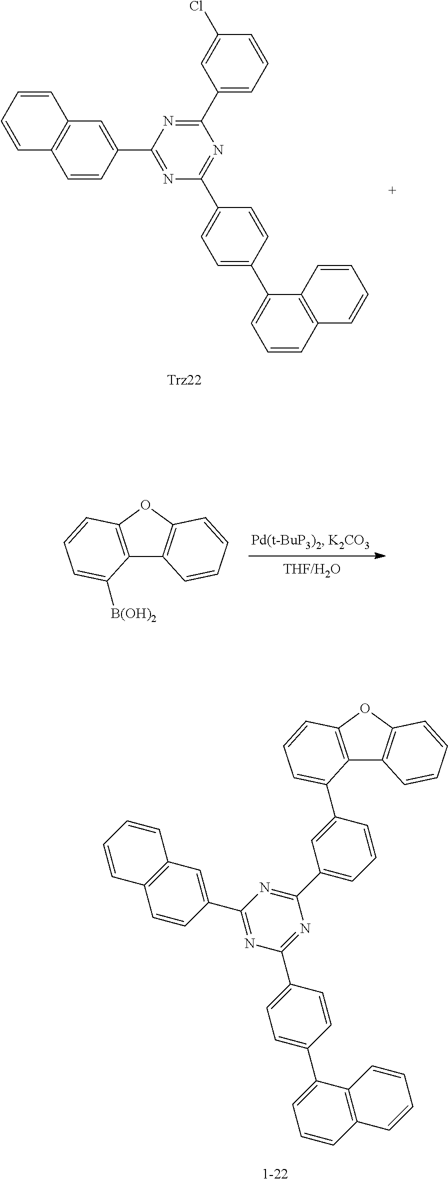 [SMILES: Clc1cccc(-c2nc(-c3ccc(-c4cccc5ccccc45)cc3)nc(-c3ccc4ccccc4c3)n2)c1 ; OB(O)c1cccc2oc3ccccc3c12 ; c1cc(-c2nc(-c3ccc(-c4cccc5ccccc45)cc3)nc(-c3ccc4ccccc4c3)n2)cc(-c2cccc3oc4ccccc4c23)c1]

Compound Trz22 (15 g, 28.8 mmol) and dibenzo[b,d]furan-1-ylboronic acid (6.4 g, 30.3 mmol) were added to 300 ml of THF, and the mixture was stirred and refluxed. Then, potassium carbonate (12 g, 86.5 mmol) was dissolved in 36 ml of water and added thereto, and the mixture was sufficiently stirred and then bis(tri-tert-butylphosphine)palladium(0) (0.1 g, 0.3 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.3 g of Compound 1-22. (Yield: 71%, MS: [M+H]⁺=652)

### Synthesis Example 1-23

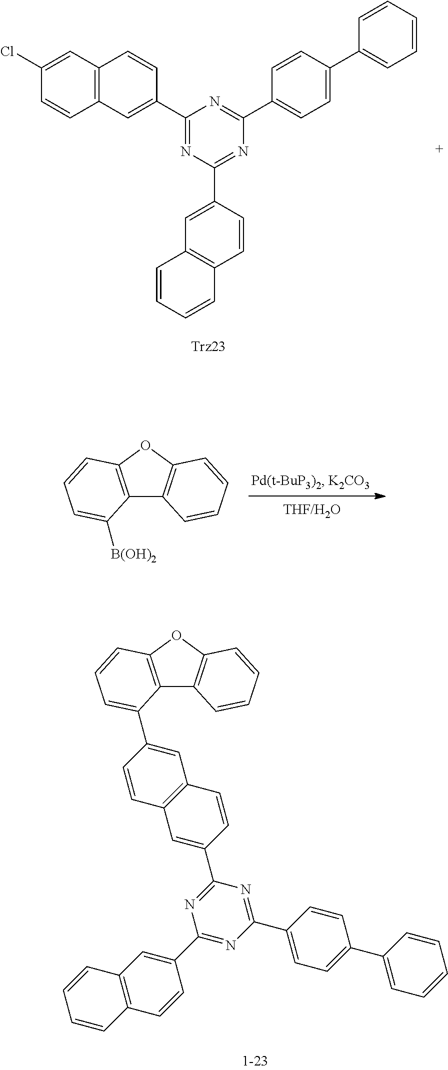 [SMILES: Clc1ccc2cc(-c3nc(-c4ccc(-c5ccccc5)cc4)nc(-c4ccc5ccccc5c4)n3)ccc2c1 ; OB(O)c1cccc2oc3ccccc3c12 ; c1ccc(-c2ccc(-c3nc(-c4ccc5ccccc5c4)nc(-c4ccc5cc(-c6cccc7oc8ccccc8c67)ccc5c4)n3)cc2)cc1]

Compound Trz23 (15 g, 28.8 mmol) and dibenzo[b,d]furan-1-ylboronic acid (6.4 g, 30.3 mmol) were added to 300 ml of THF, and the mixture was stirred and refluxed. Then, potassium carbonate (12 g, 86.5 mmol) was dissolved in 36 ml of water and added thereto, and the mixture was sufficiently stirred and then bis(tri-tert-butylphosphine)palladium(0) (0.1 g, 0.3 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.7 g of Compound 1-23. (Yield: 73%, MS: [M+H]⁺=652)

### Synthesis Example 1-24

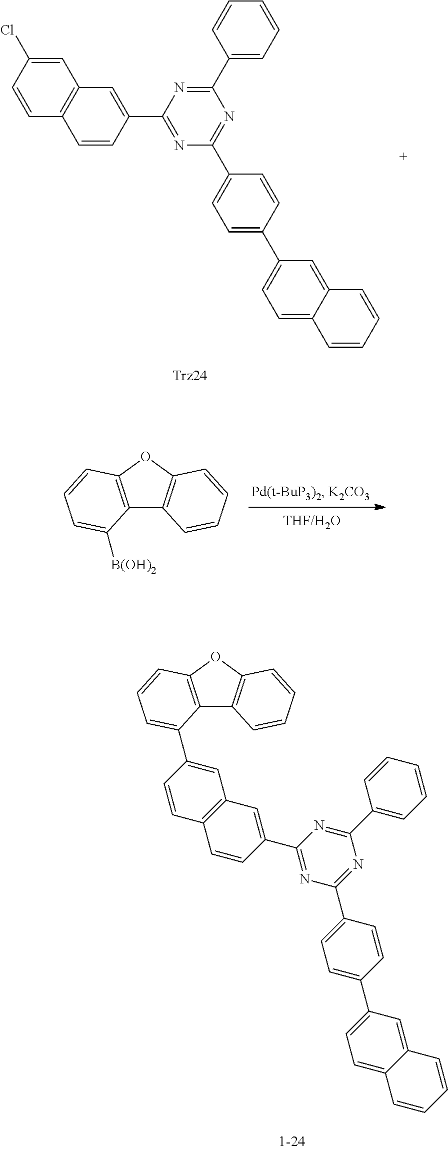 [SMILES: Clc1ccc2ccc(-c3nc(-c4ccccc4)nc(-c4ccc(-c5ccc6ccccc6c5)cc4)n3)cc2c1 ; OB(O)c1cccc2oc3ccccc3c12 ; c1ccc(-c2nc(-c3ccc(-c4ccc5ccccc5c4)cc3)nc(-c3ccc4ccc(-c5cccc6oc7ccccc7c56)cc4c3)n2)cc1]

Compound Trz24 (15 g, 28.8 mmol) and dibenzo[b,d]furan-1-ylboronic acid (6.4 g, 30.3 mmol) were added to 300 ml of THF, and the mixture was stirred and refluxed. Then, potassium carbonate (12 g, 86.5 mmol) was dissolved in 36 ml of water and added thereto, and the mixture was sufficiently stirred and then bis(tri-tert-butylphosphine)palladium(0) (0.1 g, 0.3 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.6 g of Compound 1-24. (Yield: 67%, MS: [M+H]⁺=652)

### Synthesis Example 1-25

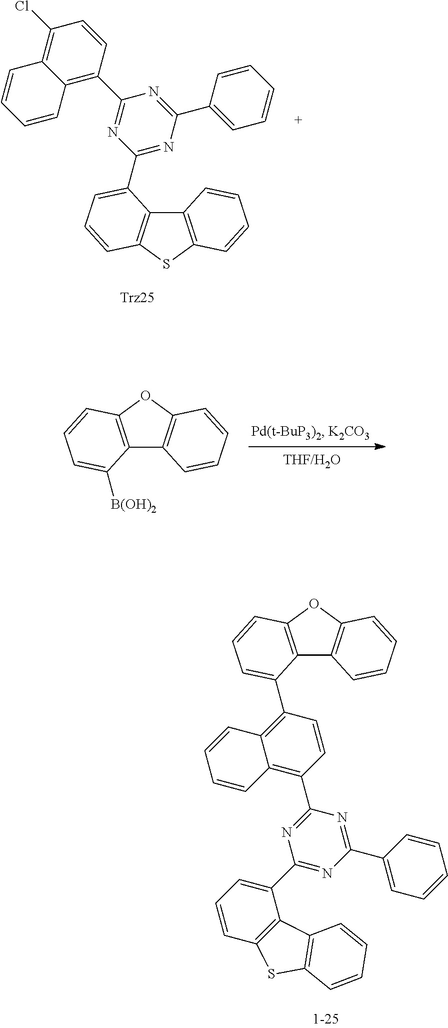 [SMILES: Clc1ccc(-c2nc(-c3ccccc3)nc(-c3cccc4sc5ccccc5c34)n2)c2ccccc12 ; OB(O)c1cccc2oc3ccccc3c12 ; c1ccc(-c2nc(-c3ccc(-c4cccc5oc6ccccc6c45)c4ccccc34)nc(-c3cccc4sc5ccccc5c34)n2)cc1]

Compound Trz25 (15 g, 30 mmol) and dibenzo[b,d]furan-1-ylboronic acid (6.7 g, 31.5 mmol) were added to 300 ml of THF, and the mixture was stirred and refluxed. Then, potassium carbonate (12.4 g, 90 mmol) was dissolved in 37 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.2 g of Compound 1-25. (Yield: 75%, MS: [M+H]⁺=632)

### Synthesis Example 1-26

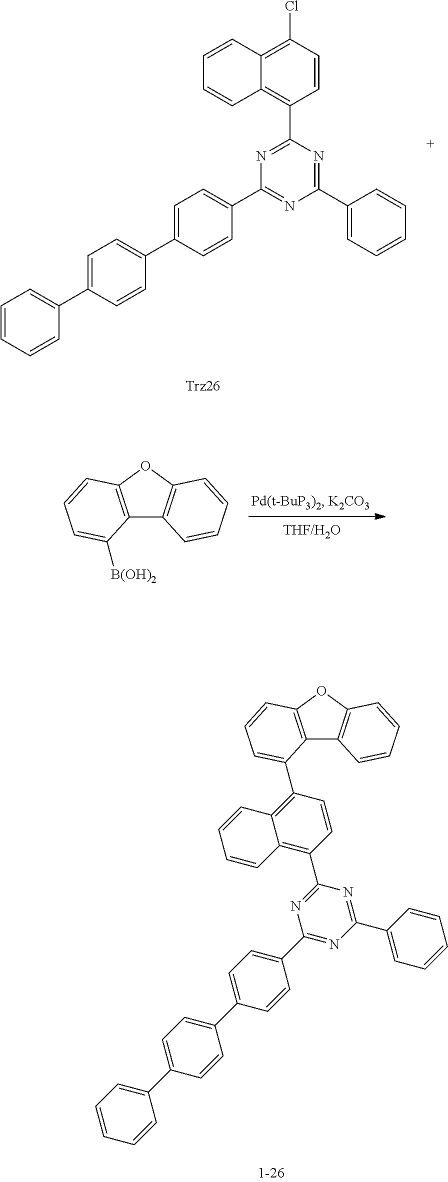 [SMILES: Clc1ccc(-c2nc(-c3ccccc3)nc(-c3ccc(-c4ccc(-c5ccccc5)cc4)cc3)n2)c2ccccc12 ; OB(O)c1cccc2oc3ccccc3c12 ; c1ccc(-c2ccc(-c3ccc(-c4nc(-c5ccccc5)nc(-c5ccc(-c6cccc7oc8ccccc8c67)c6ccccc56)n4)cc3)cc2)cc1]

Compound Trz26 (15 g, 27.5 mmol) and dibenzo[b,d]furan-1-ylboronic acid (6.1 g, 28.8 mmol) were added to 300 ml of THF, and the mixture was stirred and refluxed. Then, potassium carbonate (11.4 g, 82.4 mmol) was dissolved in 34 ml of water and added thereto, and the mixture was sufficiently stirred and then bis(tri-tert-butylphosphine)palladium(0) (0.1 g, 0.3 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14 g of Compound 1-26. (Yield: 75%, MS: [M+H]⁺=678)

### Synthesis Example 1-27

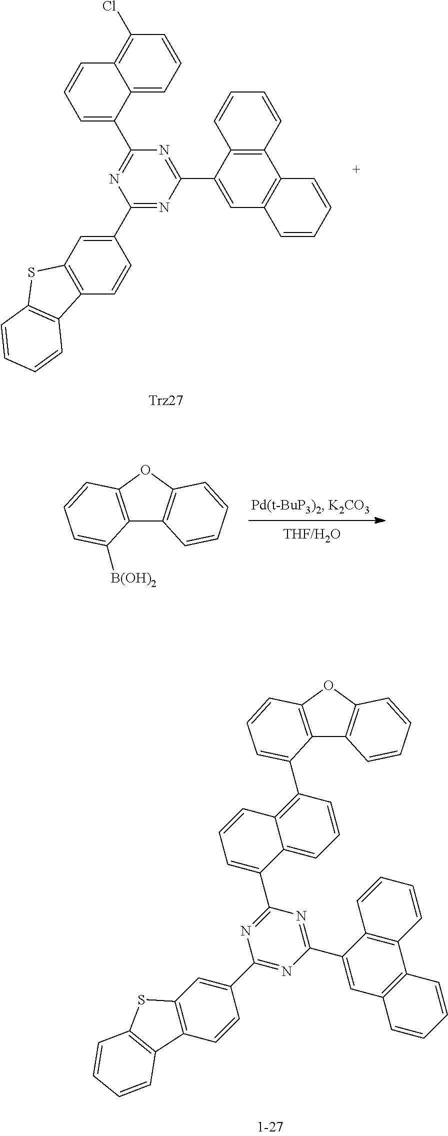 [SMILES: Clc1cccc2c(-c3nc(-c4ccc5c(c4)sc4ccccc45)nc(-c4cc5ccccc5c5ccccc45)n3)cccc12 ; OB(O)c1cccc2oc3ccccc3c12 ; c1ccc2c(c1)cc(-c1nc(-c3ccc4c(c3)sc3ccccc34)nc(-c3cccc4c(-c5cccc6oc7ccccc7c56)cccc34)n1)c1ccccc12]

Compound Trz27 (15 g, 25 mmol) and dibenzo[b,d]furan-1-ylboronic acid (5.6 g, 26.2 mmol) were added to 300 ml of THF, and the mixture was stirred and refluxed. Then, potassium carbonate (10.4 g, 75 mmol) was dissolved in 31 ml of water and added thereto, and the mixture was sufficiently stirred and then bis(tri-tert-butylphosphine)palladium(0) (0.1 g, 0.2 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.6 g of Compound 1-27. (Yield: 69%, MS: [M+H]⁺=732)

### Synthesis Example 1-28

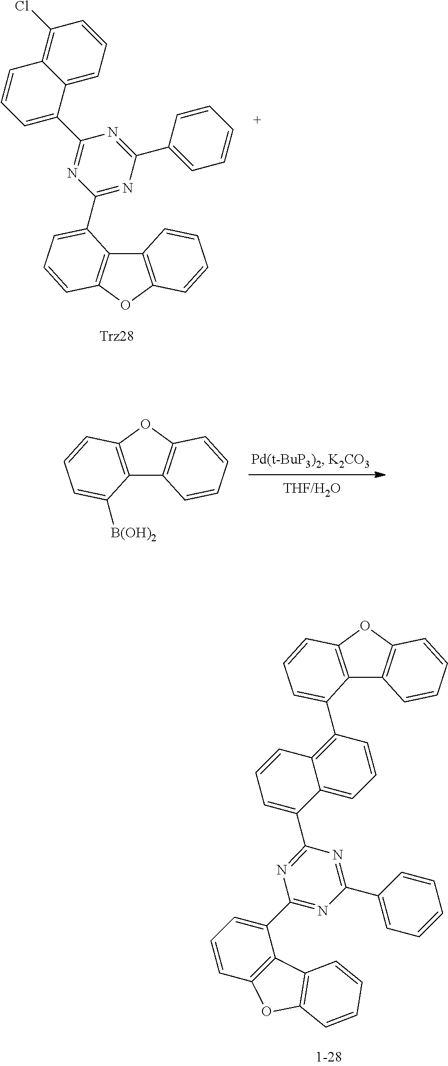

Compound Trz28 (15 g, 31 mmol) and dibenzo[b,d]furan-1-ylboronic acid (6.9 g, 32.5 mmol) were added to 300 ml of THF, and the mixture was stirred and refluxed. Then, potassium carbonate (12.9 g, 93 mmol) was dissolved in 39 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13 g of Compound 1-28. (Yield: 68%, MS: [M+H]⁺=616)

### Synthesis Example 1-29

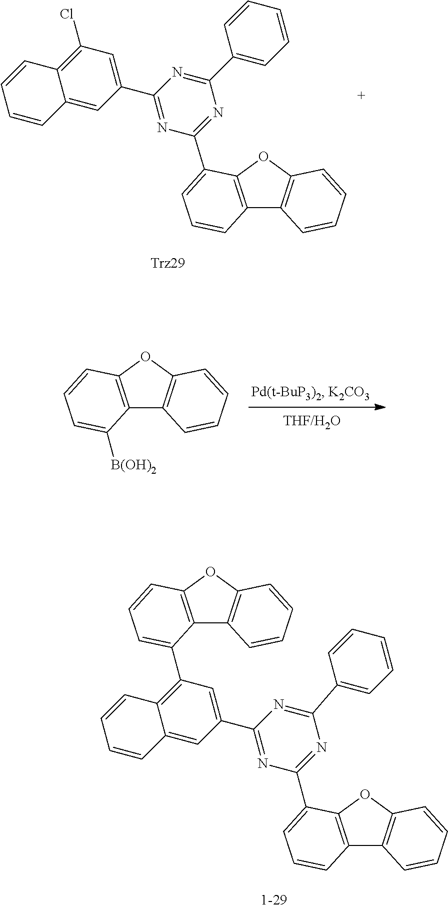 [SMILES: Clc1cc(-c2nc(-c3ccccc3)nc(-c3cccc4c3oc3ccccc34)n2)cc2ccccc12 ; OB(O)c1cccc2oc3ccccc3c12 ; c1ccc(-c2nc(-c3cc(-c4cccc5oc6ccccc6c45)c4ccccc4c3)nc(-c3cccc4c3oc3ccccc34)n2)cc1]

Compound Trz29 (15 g, 31 mmol) and dibenzo[b,d]furan-1-ylboronic acid (6.9 g, 32.5 mmol) were added to 300 ml of THF, and the mixture was stirred and refluxed. Then, potassium carbonate (12.9 g, 93 mmol) was dissolved in 39 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.3 g of Compound 1-29. (Yield: 70%, MS: [M+H]⁺=616)

### Synthesis Example 1-30

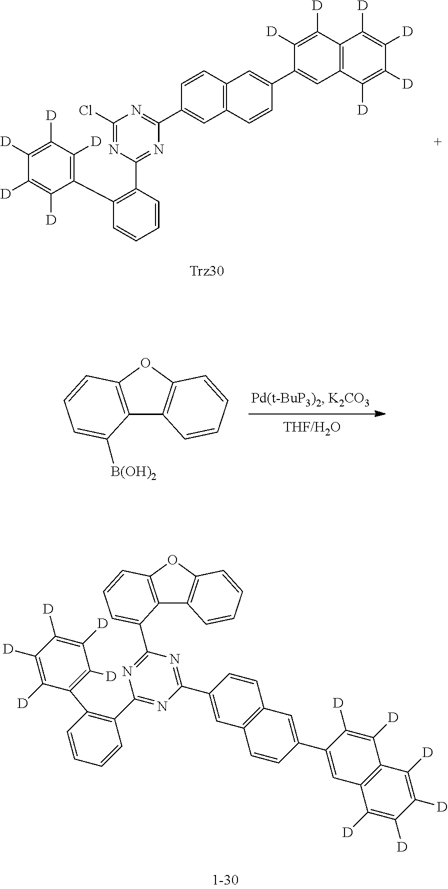 [SMILES: [2H]c1c([2H])c([2H])c(-c2ccccc2-c2nc(Cl)nc(-c3ccc4cc(-c5cc6c([2H])c([2H])c([2H])c([2H])c6c([2H])c5[2H])ccc4c3)n2)c([2H])c1[2H] ; OB(O)c1cccc2oc3ccccc3c12 ; [2H]c1c([2H])c([2H])c(-c2ccccc2-c2nc(-c3ccc4cc(-c5cc6c([2H])c([2H])c([2H])c([2H])c6c([2H])c5[2H])ccc4c3)nc(-c3cccc4oc5ccccc5c34)n2)c([2H])c1[2H]]

Compound Trz30 (15 g, 28.2 mmol) and dibenzo[b,d]furan-1-ylboronic acid (6.3 g, 29.7 mmol) were added to 300 ml of THF, and the mixture was stirred and refluxed. Then, potassium carbonate (11.7 g, 84.7 mmol) was dissolved in 35 ml of water and added thereto, and the mixture was sufficiently stirred and then bis(tri-tert-butylphosphine)palladium(0) (0.1 g, 0.3 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.9 g of Compound 1-30. (Yield: 69%, MS: [M+H]⁺=663)

### Synthesis Example 1-31

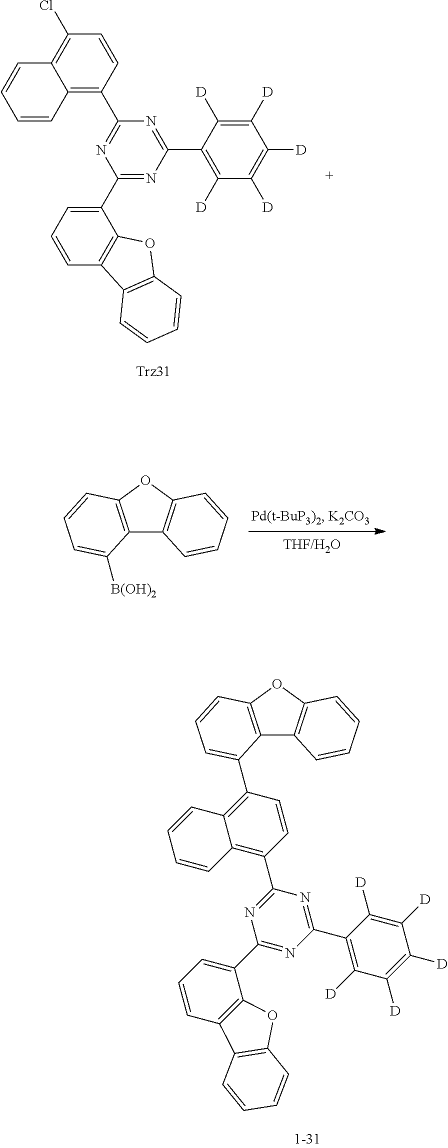 [SMILES: [2H]c1c([2H])c([2H])c(-c2nc(-c3ccc(Cl)c4ccccc34)nc(-c3cccc4c3oc3ccccc34)n2)c([2H])c1[2H] ; OB(O)c1cccc2oc3ccccc3c12 ; [2H]c1c([2H])c([2H])c(-c2nc(-c3ccc(-c4cccc5oc6ccccc6c45)c4ccccc34)nc(-c3cccc4c3oc3ccccc34)n2)c([2H])c1[2H]]

Compound Trz31 (15 g, 30.7 mmol) and dibenzo[b,d]furan-1-ylboronic acid (6.8 g, 32.2 mmol) were added to 300 ml of THF, and the mixture was stirred and refluxed. Then, potassium carbonate (12.7 g, 92 mmol) was dissolved in 38 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.3 g of Compound 1-31. (Yield: 75%, MS: [M+H]⁺=621)

### Synthesis Example 1-32

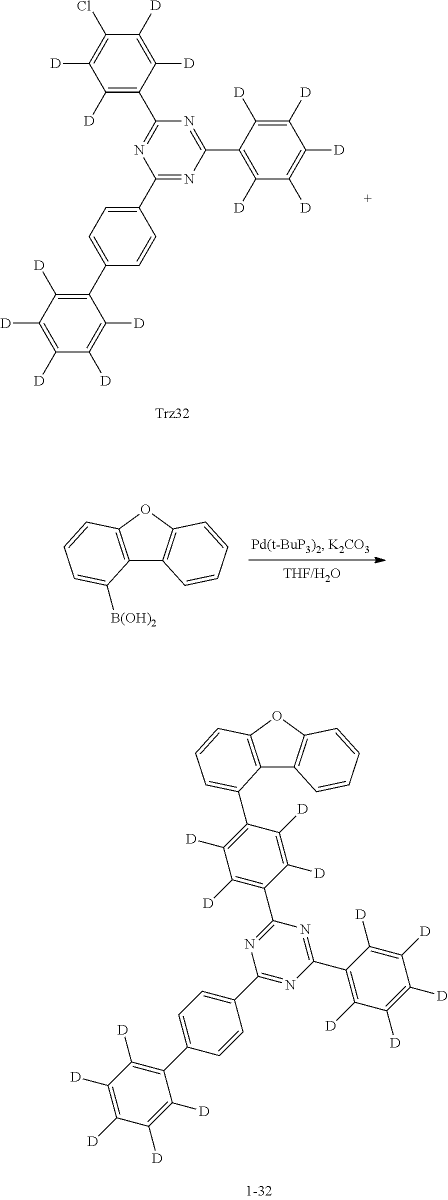 [SMILES: [2H]c1c([2H])c([2H])c(-c2ccc(-c3nc(-c4c([2H])c([2H])c([2H])c([2H])c4[2H])nc(-c4c([2H])c([2H])c(Cl)c([2H])c4[2H])n3)cc2)c([2H])c1[2H] ; OB(O)c1cccc2oc3ccccc3c12 ; [2H]c1c([2H])c([2H])c(-c2ccc(-c3nc(-c4c([2H])c([2H])c([2H])c([2H])c4[2H])nc(-c4c([2H])c([2H])c(-c5cccc6oc7ccccc7c56)c([2H])c4[2H])n3)cc2)c([2H])c1[2H]]

Compound Trz32 (15 g, 34.6 mmol) and dibenzo[b,d]furan-1-ylboronic acid (7.7 g, 36.3 mmol) were added to 300 ml of THF, and the mixture was stirred and refluxed. Then, potassium carbonate (14.3 g, 103.7 mmol) was dissolved in 43 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.9 g of Compound 1-32. (Yield: 71%, MS: [M+H]⁺=566)

### Synthesis Example 1-33

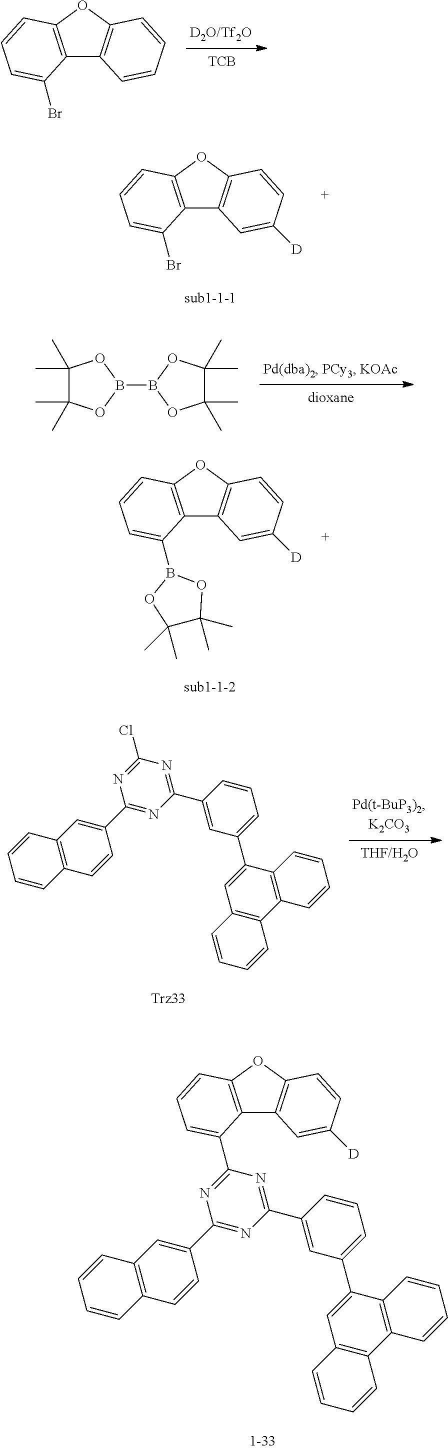

Trifluoromethanesulfonic anhydride (24 g, 85 mmol) and deuterium oxide (8.5 g, 424.9 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After reacting for 5 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.7 g of compound sub1-1-1. (Yield: 38%, MS: [M+H]⁺=248)

Compound sub1-1-1 (15 g, 60.5 mmol) and bis(pinacolato)diboron (16.9 g, 66.5 mmol) were added to 300 ml of 1,4-dioxane, and the mixture was stirred under reflux. Then, potassium acetate (8.9 g, 90.7 mmol) was added thereto, sufficiently stirred, and then bis(dibenzylideneacetone)palladium(0) (1 g, 1.8 mmol) and tricyclohexylphosphine (1 g, 3.6 mmol) were added. After reacting for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to give 13.4 g of sub1-1-2. (Yield: 75%, MS: [M+H]⁺=296)

Compound sub1-2-2 (15 g, 50.8 mmol) and Trz33 (26.4 g, 53.4 mmol) were added to 300 ml of THF, and the mixture was stirred and refluxed. Then, potassium carbonate (21.1 g, 152.5 mmol) was dissolved in 63 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21 g of Compound 1-33. (Yield: 66%, MS: [M+H]⁺=627)

### Synthesis Example 1-34

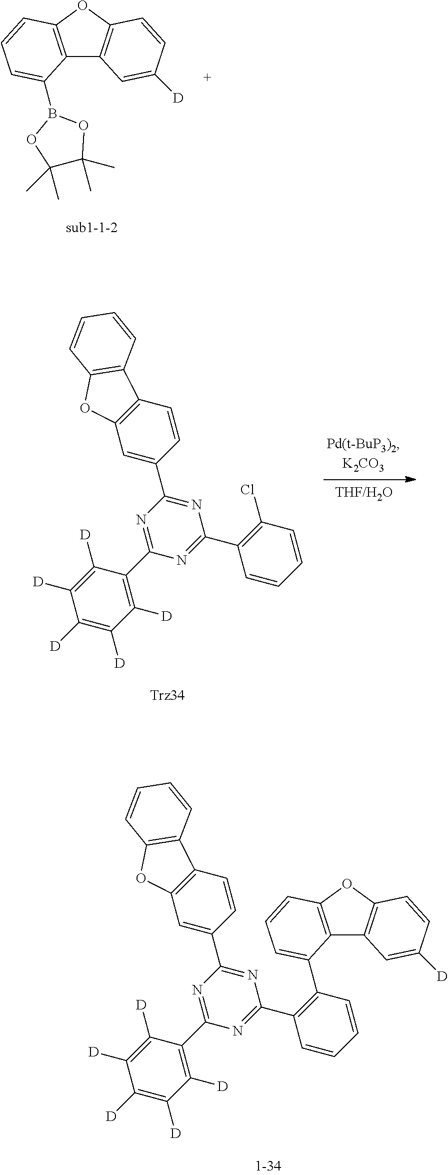 [SMILES: [2H]c1ccc2oc3cccc(B4OC(C)(C)C(C)(C)O4)c3c2c1 ; [2H]c1c([2H])c([2H])c(-c2nc(-c3ccc4c(c3)oc3ccccc34)nc(-c3ccccc3Cl)n2)c([2H])c1[2H] ; [2H]c1ccc2oc3cccc(-c4ccccc4-c4nc(-c5ccc6c(c5)oc5ccccc56)nc(-c5c([2H])c([2H])c([2H])c([2H])c5[2H])n4)c3c2c1]

Compound sub1-2-2 (15 g, 50.8 mmol) and Trz34 (23.4 g, 53.4 mmol) were added to 300 ml of THF, and the mixture was stirred and refluxed. Then, potassium carbonate (21.1 g, 152.5 mmol) was dissolved in 63 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 19.4 g of Compound 1-34. (Yield: 67%, MS: [M+H]⁺=572)

### Synthesis Example 1-35

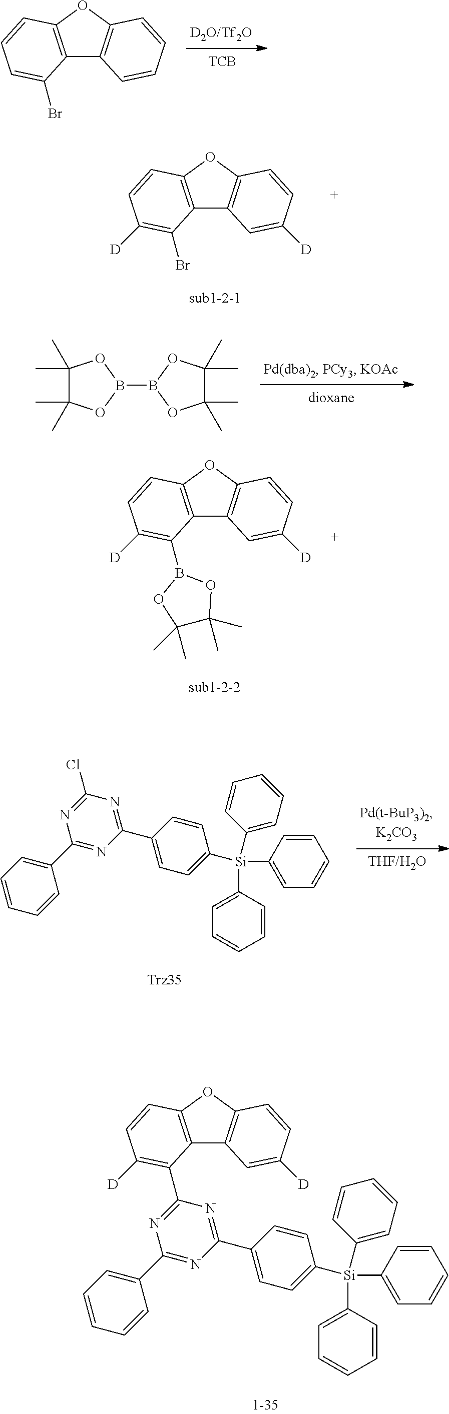

Trifluoromethanesulfonic anhydride (48 g, 170 mmol) and deuterium oxide (17 g, 849.9 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After reacting for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6 g of Compound sub1-2-1. (Yield: 40%, MS: [M+H]⁺=249)

Compound sub1-2-1 (15 g, 60.2 mmol) and bis(pinacolato)diboron (16.8 g, 66.2 mmol) were added to 300 ml of 1,4-dioxane under a nitrogen atmosphere, and the mixture was stirred under reflux. Then, potassium acetate (8.9 g, 90.3 mmol) was added thereto, sufficiently stirred, and then bis(dibenzylideneacetone)palladium(0) (1 g, 1.8 mmol) and tricyclohexylphosphine (1 g, 3.6 mmol) were added. After reacting for 4 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to give 12.5 g of sub1-2-2. (Yield: 70%, MS: [M+H]⁺=297)

Compound sub1-2-2 (15 g, 50.6 mmol) and Trz35 (28 g, 53.2 mmol) were added to 300 ml of THF, and the mixture was stirred and refluxed. Then, potassium carbonate (21 g, 151.9 mmol) was dissolved in 63 ml of water and added thereto, and the mixture was sufficiently stirred and then bis(tri-tert-butylphosphine)palladium(0) (0.3 g, 0.5 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.4 g of Compound 1-35. (Yield: 70%, MS: [M+H]⁺=660)

### Synthesis Example 1-36

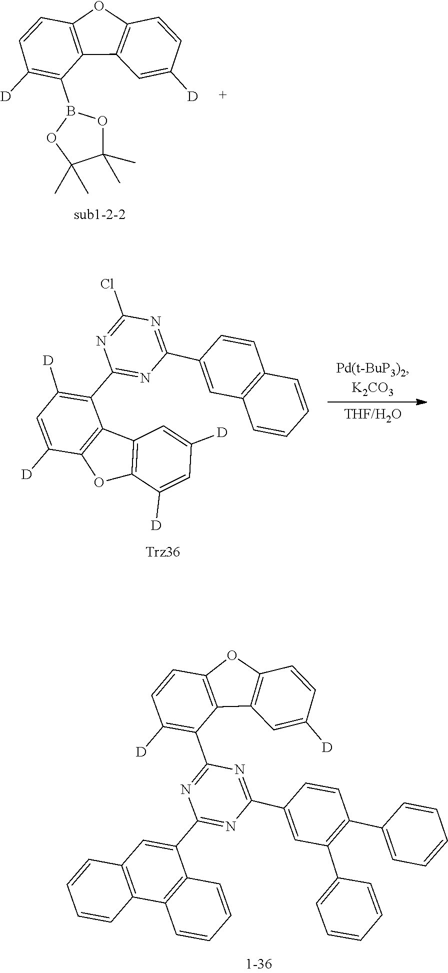 [SMILES: [2H]c1ccc2oc3ccc([2H])c(B4OC(C)(C)C(C)(C)O4)c3c2c1 ; [2H]c1cc([2H])c2oc3c([2H])cc([2H])c(-c4nc(Cl)nc(-c5ccc6ccccc6c5)n4)c3c2c1 ; [2H]c1ccc2oc3ccc([2H])c(-c4nc(-c5ccc(-c6ccccc6)c(-c6ccccc6)c5)nc(-c5cc6ccccc6c6ccccc56)n4)c3c2c1]

Compound sub1-2-2 (15 g, 50.6 mmol) and Trz36 (21.9 g, 53.2 mmol) were added to 300 ml of THF, and the mixture was stirred and refluxed. Then, potassium carbonate (21 g, 151.9 mmol) was dissolved in 63 ml of water and added thereto, and the mixture was sufficiently stirred and then bis(tri-tert-butylphosphine)palladium(0) (0.3 g, 0.5 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.5 g of Compound 1-36. (Yield: 68%, MS: [M+H]⁺=654)

### Synthesis Example 1-37

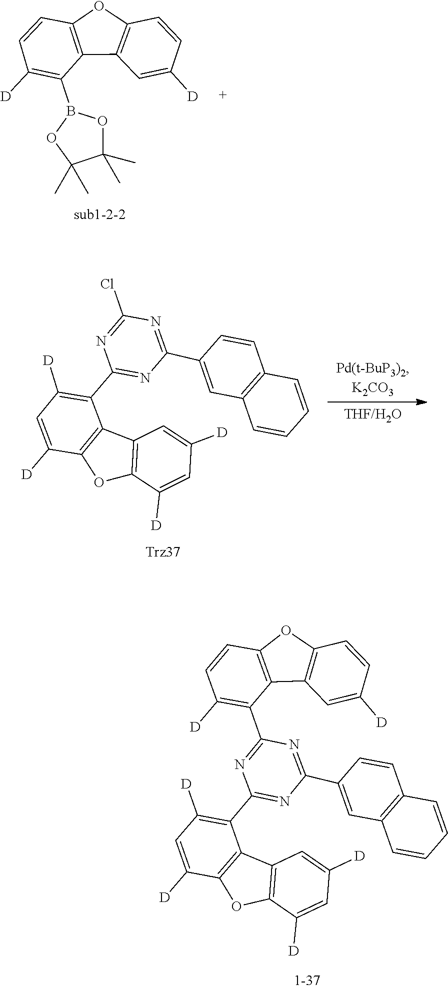 [SMILES: [2H]c1ccc2oc3ccc([2H])c(B4OC(C)(C)C(C)(C)O4)c3c2c1 ; [2H]c1cc([2H])c2oc3c([2H])cc([2H])c(-c4nc(Cl)nc(-c5ccc6ccccc6c5)n4)c3c2c1 ; [2H]c1ccc2oc3ccc([2H])c(-c4nc(-c5ccc6ccccc6c5)nc(-c5c([2H])cc([2H])c6oc7c([2H])cc([2H])cc7c56)n4)c3c2c1]

Compound sub1-2-2 (15 g, 50.6 mmol) and Trz37 (21.9 g, 53.2 mmol) were added to 300 ml of THF, and the mixture was stirred and refluxed. Then, potassium carbonate (21 g, 151.9 mmol) was dissolved in 63 ml of water and added thereto, and the mixture was sufficiently stirred and then bis(tri-tert-butylphosphine)palladium(0) (0.3 g, 0.5 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 17.9 g of Compound 1-37. (Yield: 65%, MS: [M+H]⁺=546)

### Synthesis Example 1-38

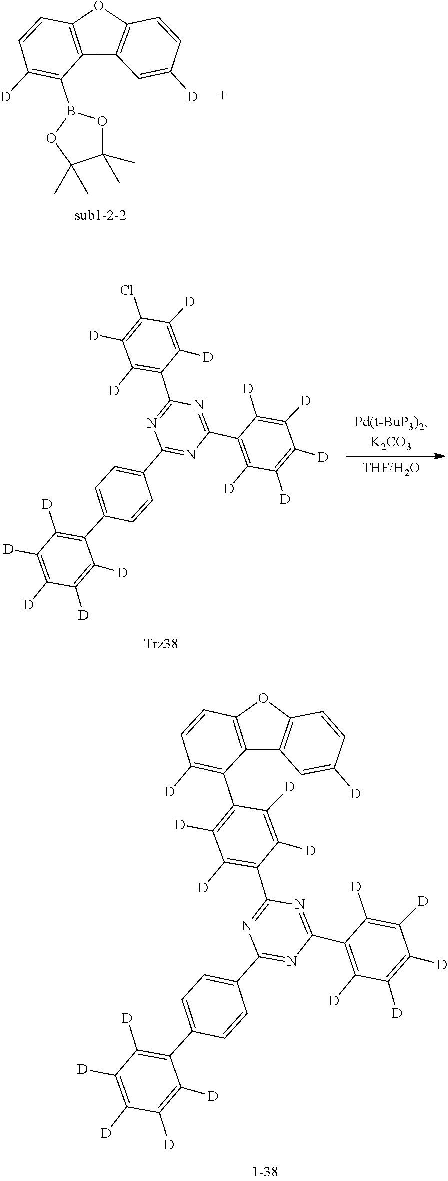 [SMILES: [2H]c1ccc2oc3ccc([2H])c(B4OC(C)(C)C(C)(C)O4)c3c2c1 ; [2H]c1c([2H])c([2H])c(-c2ccc(-c3nc(-c4c([2H])c([2H])c([2H])c([2H])c4[2H])nc(-c4c([2H])c([2H])c(Cl)c([2H])c4[2H])n3)cc2)c([2H])c1[2H] ; [2H]c1ccc2oc3ccc([2H])c(-c4c([2H])c([2H])c(-c5nc(-c6ccc(-c7c([2H])c([2H])c([2H])c([2H])c7[2H])cc6)nc(-c6c([2H])c([2H])c([2H])c([2H])c6[2H])n5)c([2H])c4[2H])c3c2c1]

Compound sub1-2-2 (15 g, 50.6 mmol) and Trz38 (23.1 g, 53.2 mmol) were added to 300 ml of THF, and the mixture was stirred and refluxed. Then, potassium carbonate (21 g, 151.9 mmol) was dissolved in 63 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 19 g of Compound 1-38. (Yield: 66%, MS: [M+H]⁺=568)

### Synthesis Example 1-39

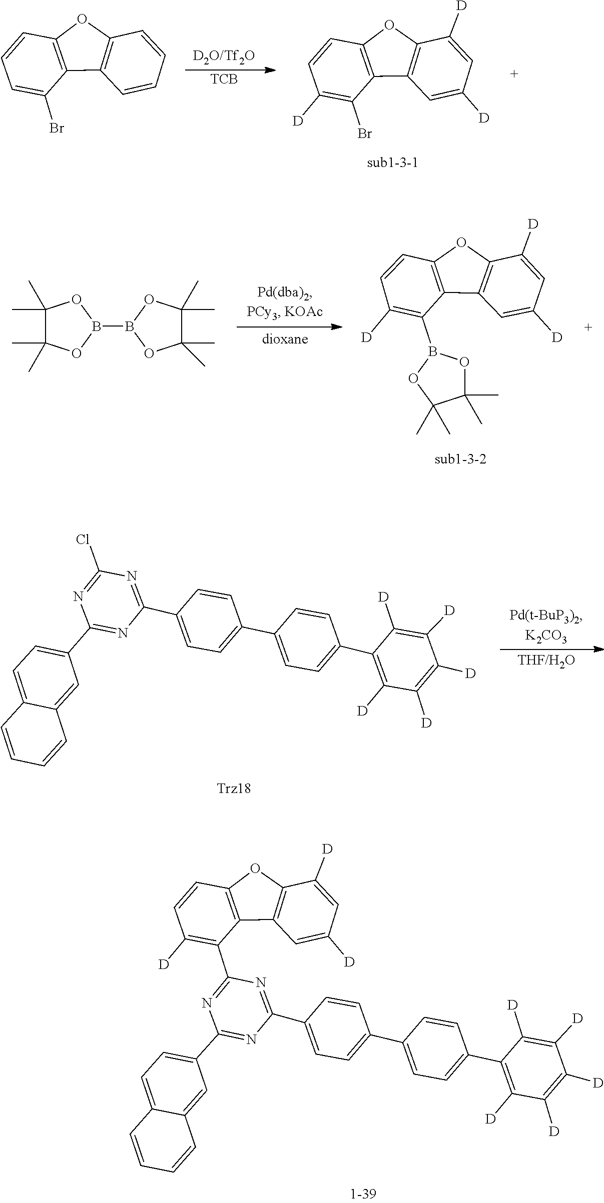

Trifluoromethanesulfonic anhydride (71.9 g, 255 mmol) and deuterium oxide (25.5 g, 1274.8 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After reacting for 14 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.3 g of compound sub1-3-1. (Yield: 42%, MS: [M+H]⁺=250)

Compound sub1-3-1 (15 g, 60 mmol) and bis(pinacolato)diboron (16.8 g, 66 mmol) were added to 300 ml of 1,4-dioxane, and the mixture was stirred under reflux. Then, potassium acetate (8.8 g, 90 mmol) was added thereto, sufficiently stirred, and then bis(dibenzylideneacetone)palladium(0) (1 g, 1.8 mmol) and tricyclohexylphosphine (1 g, 3.6 mmol) were added. After reacting for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to give 11.4 g of sub1-3-2. (Yield: 64%, MS: [M+H]⁺=298) Compound sub1-3-2 (15 g, 50.5 mmol) and Trz18 (25.2 g, 53 mmol) were added to 300 ml of THF, and the mixture was stirred and refluxed. Then, potassium carbonate (20.9 g, 151.4 mmol) was dissolved in 63 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.1 g of Compound 1-39. (Yield: 75%, MS: [M+H]⁺=610)

### Synthesis Example 1-40

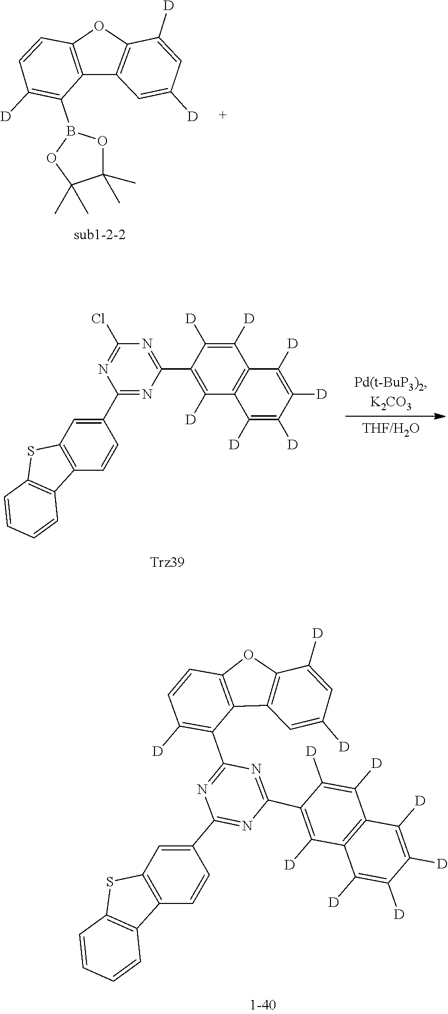 [SMILES: [2H]c1cc([2H])c2oc3ccc([2H])c(B4OC(C)(C)C(C)(C)O4)c3c2c1 ; [2H]c1c([2H])c([2H])c2c([2H])c(-c3nc(Cl)nc(-c4ccc5c(c4)sc4ccccc45)n3)c([2H])c([2H])c2c1[2H] ; [2H]c1cc([2H])c2oc3ccc([2H])c(-c4nc(-c5ccc6c(c5)sc5ccccc56)nc(-c5c([2H])c([2H])c6c([2H])c([2H])c([2H])c([2H])c6c5[2H])n4)c3c2c1]

Compound sub1-3-2 (15 g, 50.5 mmol) and Trz39 (22.8 g, 53 mmol) were added to 300 ml of THF, and the mixture was stirred and refluxed. Then, potassium carbonate (20.9 g, 151.4 mmol) was dissolved in 63 ml of water and added thereto, and the mixture was sufficiently stirred and then bis(tri-tert-butylphosphine)palladium(0) (0.3 g, 0.5 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 18.5 g of Compound 1-40. (Yield: 65%, MS: [M+H]⁺=565)

### Synthesis Example 1-41

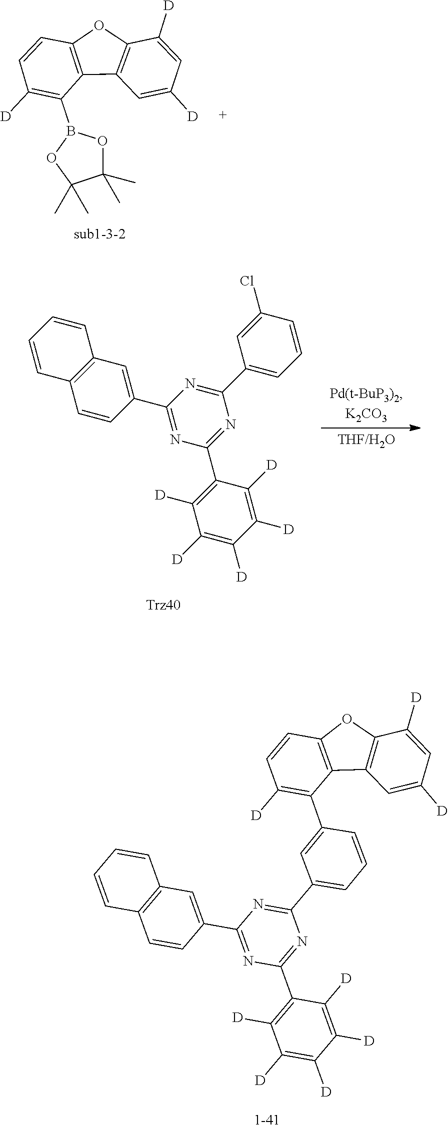 [SMILES: [2H]c1cc([2H])c2oc3ccc([2H])c(B4OC(C)(C)C(C)(C)O4)c3c2c1 ; [2H]c1c([2H])c([2H])c(-c2nc(-c3cccc(Cl)c3)nc(-c3ccc4ccccc4c3)n2)c([2H])c1[2H] ; [2H]c1cc([2H])c2oc3ccc([2H])c(-c4cccc(-c5nc(-c6ccc7ccccc7c6)nc(-c6c([2H])c([2H])c([2H])c([2H])c6[2H])n5)c4)c3c2c1]

Compound sub1-3-2 (15 g, 50.5 mmol) and Trz40 (21.1 g, 53 mmol) were added to 300 ml of THF, and the mixture was stirred and refluxed. Then, potassium carbonate (20.9 g, 151.4 mmol) was dissolved in 63 ml of water and added thereto, and the mixture was sufficiently stirred and then bis(tri-tert-butylphosphine)balladium(0) (0.3 g, 0.5 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 17.8 g of Compound 1-41. (Yield: 66%, MS: [M+H]⁺=534)

### Synthesis Example 1-42

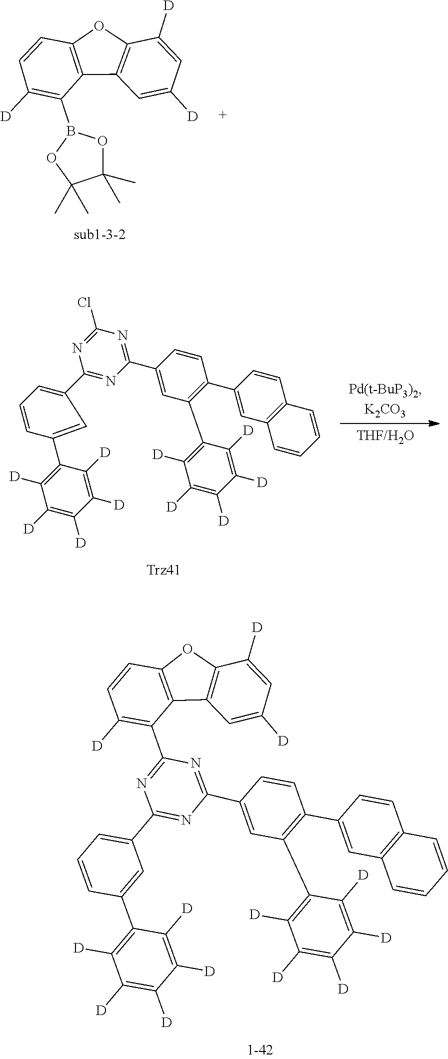 [SMILES: [2H]c1cc([2H])c2oc3ccc([2H])c(B4OC(C)(C)C(C)(C)O4)c3c2c1 ; [2H]c1c([2H])c([2H])c(-c2cccc(-c3nc(Cl)nc(-c4ccc(-c5ccc6ccccc6c5)c(-c5c([2H])c([2H])c([2H])c([2H])c5[2H])c4)n3)c2)c([2H])c1[2H] ; [2H]c1cc([2H])c2oc3ccc([2H])c(-c4nc(-c5cccc(-c6c([2H])c([2H])c([2H])c([2H])c6[2H])c5)nc(-c5ccc(-c6ccc7ccccc7c6)c(-c6c([2H])c([2H])c([2H])c([2H])c6[2H])c5)n4)c3c2c1]

Compound sub1-3-2 (15 g, 50.5 mmol) and Trz41 (29.5 g, 53 mmol) were added to 300 ml of THF, and the mixture was stirred and refluxed. Then, potassium carbonate (20.9 g, 151.4 mmol) was dissolved in 63 ml of water and added thereto, and the mixture was sufficiently stirred and then bis(tri-tert-butylphosphine)palladium(0) (0.3 g, 0.5 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.4 g of Compound 1-42. (Yield: 70%, MS: [M+H]⁺=691)

### Synthesis Example 1-43

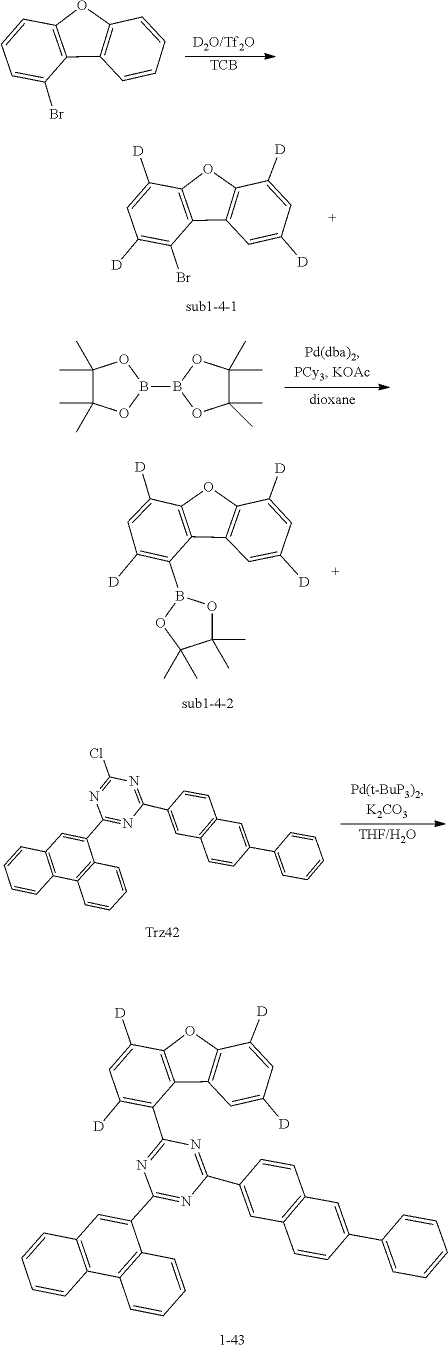

Trifluoromethanesulfonic anhydride (95.9 g, 340 mmol) and deuterium oxide (34 g, 1699.8 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After reacting for 20 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.6 g of compound sub1-4-1. (Yield: 37%, MS: [M+H]⁺=251)

Compound sub1-4-1 (15 g, 59.7 mmol) and bis(pinacolato)diboron (16.7 g, 65.7 mmol) were added to 300 ml of 1,4-dioxane under a nitrogen atmosphere, and the mixture was stirred under reflux. Then, potassium acetate (8.8 g, 89.6 mmol) was added thereto, sufficiently stirred, and then bis(dibenzylideneacetone)palladium(0) (1 g, 1.8 mmol) and tricyclohexylphosphine (1 g, 3.6 mmol) were added. After reacting for 5 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to give 12.5 g of sub1-4-2. (Yield: 70%, MS: [M+H]⁺=299)

Compound sub1-4-2 (15 g, 50.3 mmol) and Trz42 (26.1 g, 52.8 mmol) were added to 300 ml of THF, and the mixture was stirred and refluxed. Then, potassium carbonate (20.9 g, 150.9 mmol) was dissolved in 63 ml of water and added thereto, and the mixture was sufficiently stirred and then bis(tri-tert-butylphosphine)palladium(0) (0.3 g, 0.5 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.5 g of Compound 1-43. (Yield: 68%, MS: [M+H]⁺=631)

### Synthesis Example 1-44

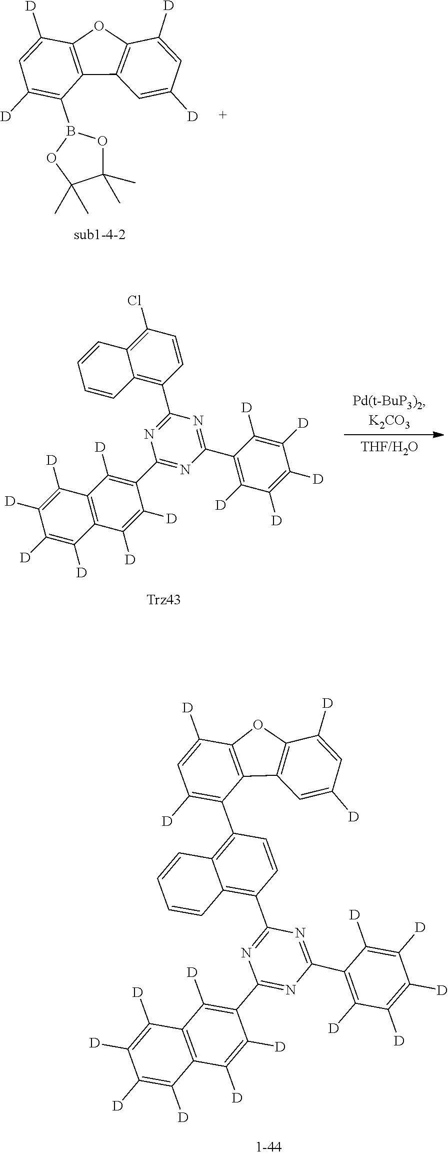 [SMILES: [2H]c1cc([2H])c2oc3c([2H])cc([2H])c(B4OC(C)(C)C(C)(C)O4)c3c2c1 ; [2H]c1c([2H])c([2H])c(-c2nc(-c3c([2H])c([2H])c4c([2H])c([2H])c([2H])c([2H])c4c3[2H])nc(-c3ccc(Cl)c4ccccc34)n2)c([2H])c1[2H] ; [2H]c1cc([2H])c2oc3c([2H])cc([2H])c(-c4ccc(-c5nc(-c6c([2H])c([2H])c([2H])c([2H])c6[2H])nc(-c6c([2H])c([2H])c7c([2H])c([2H])c([2H])c([2H])c7c6[2H])n5)c5ccccc45)c3c2c1]

Compound sub1-4-2 (15 g, 50.3 mmol) and Trz43 (24.1 g, 52.8 mmol) were added to 300 ml of THF, and the mixture was stirred and refluxed. Then, potassium carbonate (20.9 g, 150.9 mmol) was dissolved in 63 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.2 g of Compound 1-44. (Yield: 68%, MS: [M+H]⁺=592)

### Synthesis Example 1-45

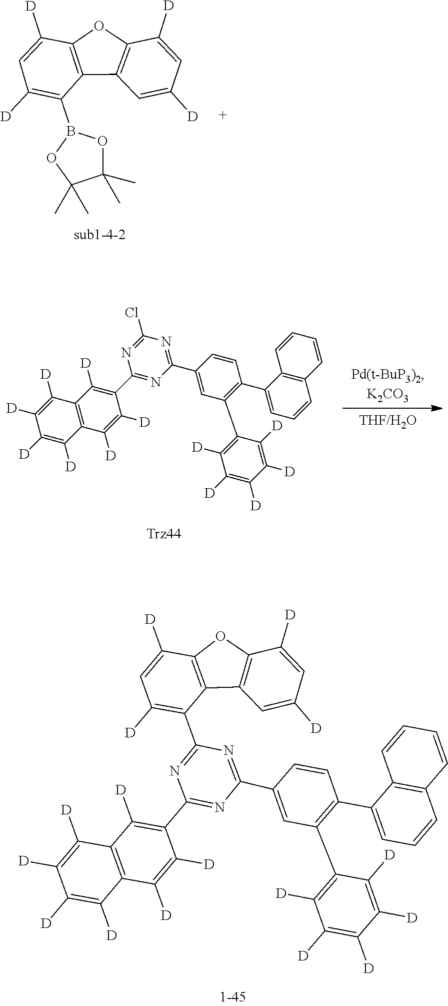 [SMILES: [2H]c1cc([2H])c2oc3c([2H])cc([2H])c(B4OC(C)(C)C(C)(C)O4)c3c2c1 ; [2H]c1c([2H])c([2H])c(-c2cc(-c3nc(Cl)nc(-c4c([2H])c([2H])c5c([2H])c([2H])c([2H])c([2H])c5c4[2H])n3)ccc2-c2cccc3ccccc23)c([2H])c1[2H] ; [2H]c1cc([2H])c2oc3c([2H])cc([2H])c(-c4nc(-c5ccc(-c6cccc7ccccc67)c(-c6c([2H])c([2H])c([2H])c([2H])c6[2H])c5)nc(-c5c([2H])c([2H])c6c([2H])c([2H])c([2H])c([2H])c6c5[2H])n4)c3c2c1]

Compound sub1-4-2 (15 g, 50.3 mmol) and Trz44 (28.1 g, 52.8 mmol) were added to 300 ml of THE, and the mixture was stirred and refluxed. Then, potassium carbonate (20.9 g, 150.9 mmol) was dissolved in 63 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.2 g of Compound 1-45. (Yield: 72%, MS: [M+H]⁺=668)

### Synthesis Example 1-46

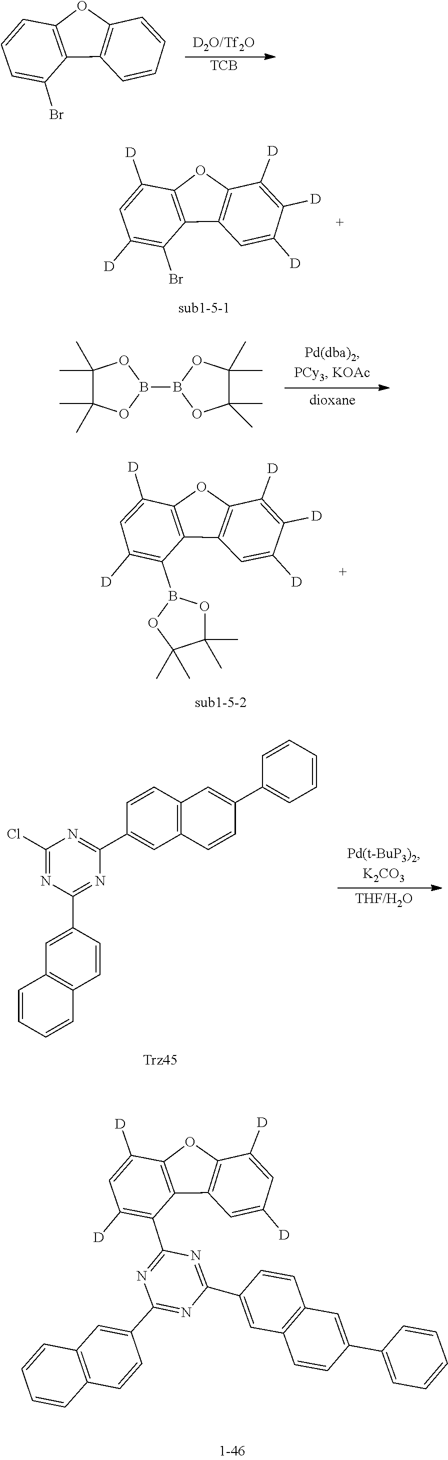

Trifluoromethanesulfonic anhydride (119.9 g, 424.9 mmol) and deuterium oxide (42.6 g, 2124.7 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After reacting for 24 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.9 g of compound sub1-5-1. (Yield: 39%, MS: [M+H]⁺=252)

Compound sub1-5-1 (15 g, 59.5 mmol) and bis(pinacolato)diboron (16.6 g, 65.4 mmol) were added to 300 ml of 1,4-dioxane, and the mixture was stirred under reflux. Then, potassium acetate (8.8 g, 89.2 mmol) was added thereto, sufficiently stirred, and then bis(dibenzylideneacetone)palladium(0) (1 g, 1.8 mmol) and tricyclohexylphosphine (1 g, 3.6 mmol) were added. After reacting for 4 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to give 11.2 g of sub1-(Yield: 63%, MS: [M+H]⁺=300)

Compound sub1-5-2 (15 g, 50.1 mmol) and Trz45 (23.4 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred and then bis(tri-tert-butylphosphine)palladium(0) (0.3 g, 0.5 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.1 g of Compound 1-46. (Yield: 69%, MS: [M+H]⁺=581)

### Synthesis Example 1-47

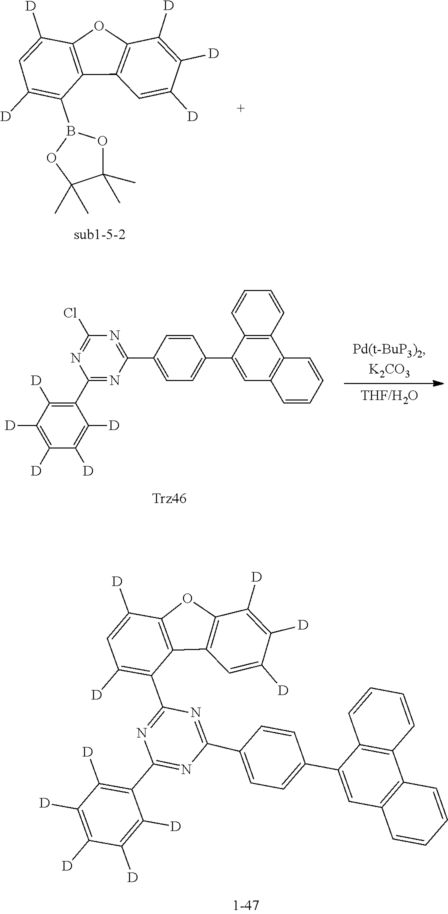 [SMILES: [2H]c1cc2c(oc3c([2H])cc([2H])c(B4OC(C)(C)C(C)(C)O4)c32)c([2H])c1[2H] ; [2H]c1c([2H])c([2H])c(-c2nc(Cl)nc(-c3ccc(-c4cc5ccccc5c5ccccc45)cc3)n2)c([2H])c1[2H] ; [2H]c1cc2c(oc3c([2H])cc([2H])c(-c4nc(-c5ccc(-c6cc7ccccc7c7ccccc67)cc5)nc(-c5c([2H])c([2H])c([2H])c([2H])c5[2H])n4)c32)c([2H])c1[2H]]

Compound sub1-5-2 (15 g, 50.1 mmol) and Compound Trz46 (23.6 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred and then bis(tri-tert-butylphosphine)palladium(0) (0.3 g, 0.5 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.2 g of Compound 1-47. (Yield: 69%, MS: [M+H]⁺=586)

### Synthesis Example 1-48

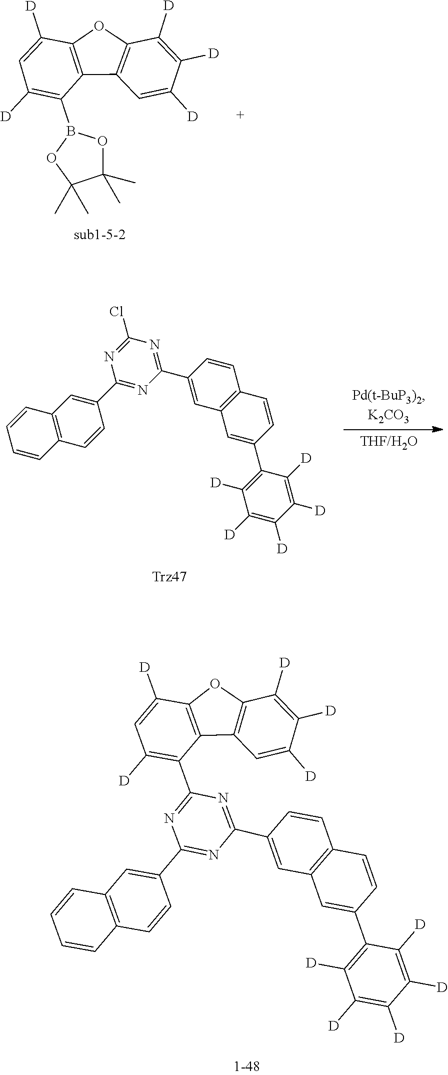 [SMILES: [2H]c1cc2c(oc3c([2H])cc([2H])c(B4OC(C)(C)C(C)(C)O4)c32)c([2H])c1[2H] ; [2H]c1c([2H])c([2H])c(-c2ccc3ccc(-c4nc(Cl)nc(-c5ccc6ccccc6c5)n4)cc3c2)c([2H])c1[2H] ; [2H]c1cc2c(oc3c([2H])cc([2H])c(-c4nc(-c5ccc6ccccc6c5)nc(-c5ccc6ccc(-c7c([2H])c([2H])c([2H])c([2H])c7[2H])cc6c5)n4)c32)c([2H])c1[2H]]

Compound sub1-5-2 (15 g, 50.1 mmol) and Compound Trz47 (23.6 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred and then bis(tri-tert-butylphosphine)palladium(0) (0.3 g, 0.5 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.7 g of Compound 1-48. (Yield: 74%, MS: [M+H]⁺=586)

### Synthesis Example 1-49

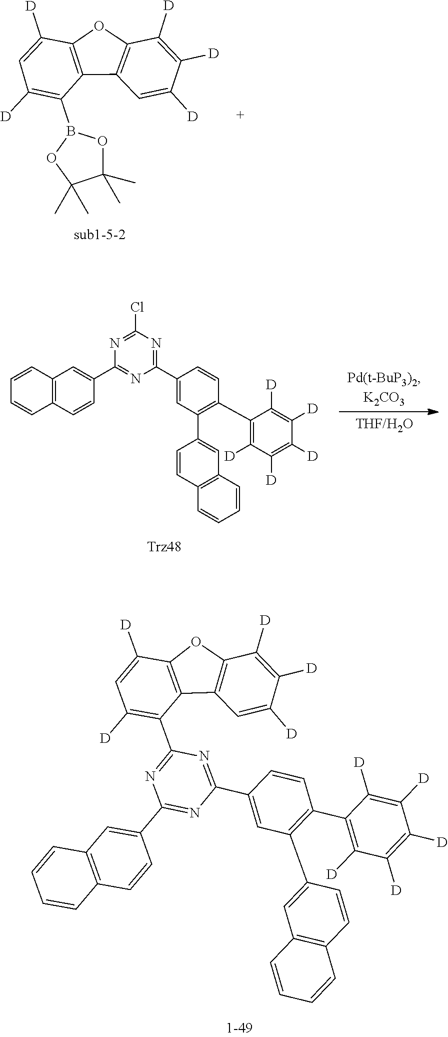 [SMILES: [2H]c1cc2c(oc3c([2H])cc([2H])c(B4OC(C)(C)C(C)(C)O4)c32)c([2H])c1[2H] ; [2H]c1c([2H])c([2H])c(-c2ccc(-c3nc(Cl)nc(-c4ccc5ccccc5c4)n3)cc2-c2ccc3ccccc3c2)c([2H])c1[2H] ; [2H]c1cc2c(oc3c([2H])cc([2H])c(-c4nc(-c5ccc(-c6c([2H])c([2H])c([2H])c([2H])c6[2H])c(-c6ccc7ccccc7c6)c5)nc(-c5ccc6ccccc6c5)n4)c32)c([2H])c1[2H]]

Compound sub1-5-2 (15 g, 50.1 mmol) and Compound Trz48 (27.6 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred and then bis(tri-tert-butylphosphine)palladium(0) (0.3 g, 0.5 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.5 g of Compound 1-49. (Yield: 68%, MS: [M+H]⁺=662)

### Synthesis Example 1-50

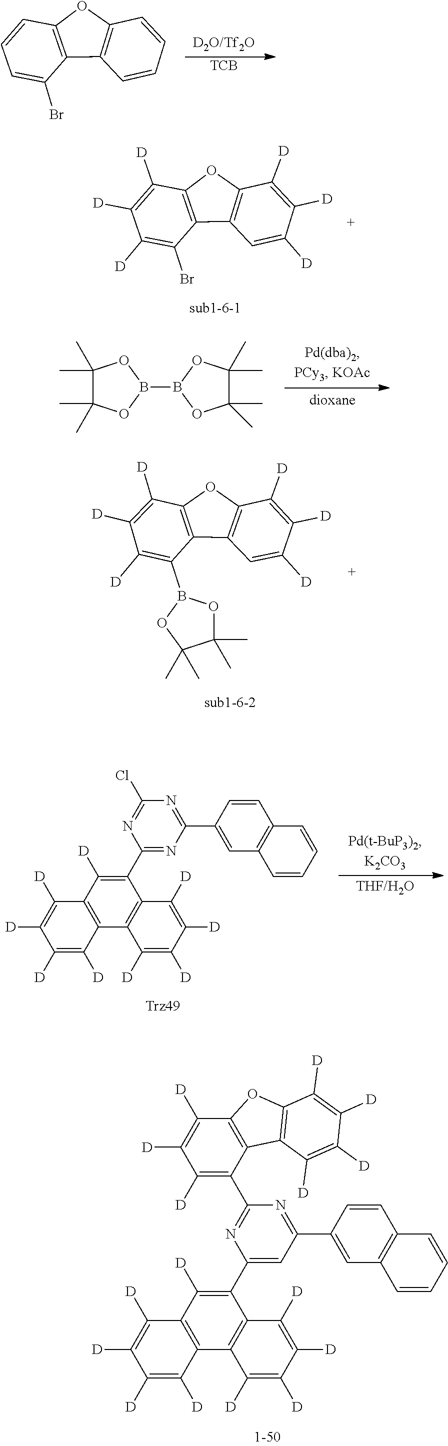

Trifluoromethanesulfonic anhydride (167.8 g, 594.9 mmol) and deuterium oxide (59.6 g, 2974.6 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After reacting for 36 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.1 g of Compound sub1-6-1. (Yield: 40%, MS: [M+H]⁺=254)

Compound sub1-6-1 (15 g, 59 mmol) and bis(pinacolato)diboron (16.5 g, 64.9 mmol) were added to 300 ml of 1,4-dioxane under a nitrogen atmosphere, and the mixture was stirred under reflux. Then, potassium acetate (8.7 g, 88.5 mmol) was added thereto, sufficiently stirred, and then bis(dibenzylideneacetone)palladium(0) (1 g, 1.8 mmol) and tricyclohexylphosphine (1 g, 3.5 mmol) were added. After reacting for 4 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to give 11.6 g of Compound sub1-6-2. (Yield: 65%, MS: [M+H]⁺=302)

Compound sub1-6-2 (15 g, 49.8 mmol) and Compound Trz49 (22.3 g, 52.3 mmol) were added to 300 ml of THF, and the mixture was stirred and refluxed. Then, potassium carbonate (20.6 g, 149.4 mmol) was dissolved in 62 ml of water and added thereto, and the mixture was sufficiently stirred and then bis(tri-tert-butylphosphine)palladium(0) (0.3 g, 0.5 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.3 g of Compound 1-50. (Yield: 72%, MS: [M+H]⁺=566)

### Synthesis Example 1-51

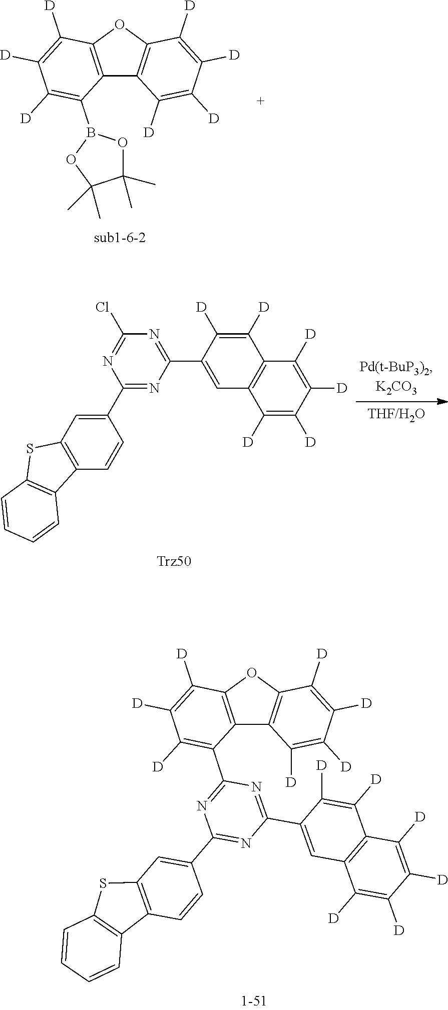 [SMILES: [2H]c1c([2H])c([2H])c2c(oc3c([2H])c([2H])c([2H])c(B4OC(C)(C)C(C)(C)O4)c32)c1[2H] ; [2H]c1c([2H])c([2H])c2c([2H])c([2H])c(-c3nc(Cl)nc(-c4ccc5c(c4)sc4ccccc45)n3)cc2c1[2H] ; [2H]c1c([2H])c([2H])c2c([2H])c([2H])c(-c3nc(-c4ccc5c(c4)sc4ccccc45)nc(-c4c([2H])c([2H])c([2H])c5oc6c([2H])c([2H])c([2H])c([2H])c6c45)n3)cc2c1[2H]]

Compound sub1-6-2 (15 g, 49.8 mmol) and Compound Trz50 (22.5 g, 52.3 mmol) were added to 300 ml of THF, and the mixture was stirred and refluxed. Then, potassium carbonate (20.6 g, 149.4 mmol) was dissolved in 62 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.4 g of Compound 1-51. (Yield: 72%, MS: [M+H]⁺=569)

### Synthesis Example 1-52

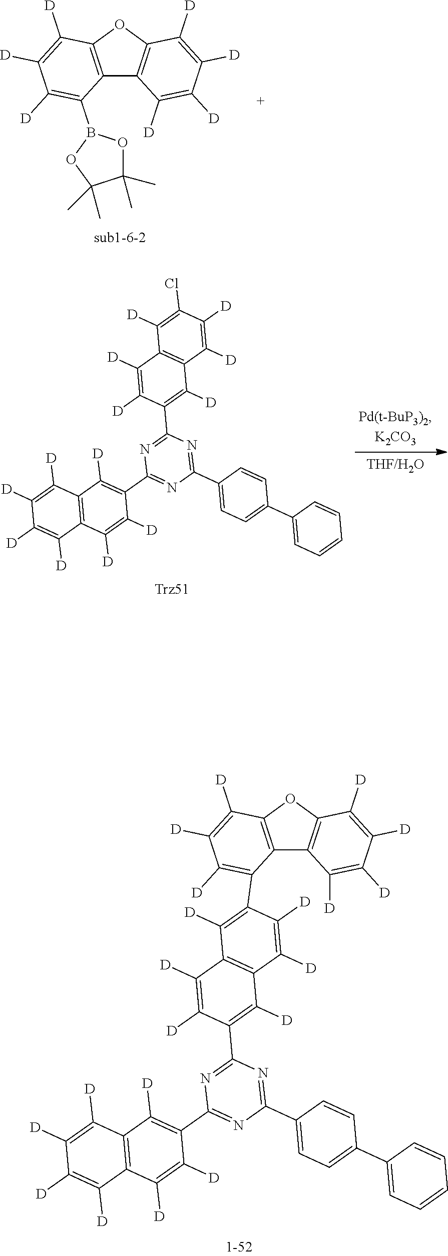 [SMILES: [2H]c1c([2H])c([2H])c2c(oc3c([2H])c([2H])c([2H])c(B4OC(C)(C)C(C)(C)O4)c32)c1[2H] ; [2H]c1c([2H])c([2H])c2c([2H])c(-c3nc(-c4ccc(-c5ccccc5)cc4)nc(-c4c([2H])c([2H])c5c([2H])c(Cl)c([2H])c([2H])c5c4[2H])n3)c([2H])c([2H])c2c1[2H] ; [2H]c1c([2H])c([2H])c2c(oc3c([2H])c([2H])c([2H])c(-c4c([2H])c([2H])c5c([2H])c(-c6nc(-c7ccc(-c8ccccc8)cc7)nc(-c7c([2H])c([2H])c8c([2H])c([2H])c([2H])c([2H])c8c7[2H])n6)c([2H])c([2H])c5c4[2H])c32)c1[2H]]

Compound sub1-6-2 (15 g, 49.8 mmol) and Compound Trz51 (27.9 g, 52.3 mmol) were added to 300 ml of THF, and the mixture was stirred and refluxed. Then, potassium carbonate (20.6 g, 149.4 mmol) was dissolved in 62 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.7 g of Compound 1-52. (Yield: 74%, MS: [M+H]⁺=672)

### Synthesis Example 1-53

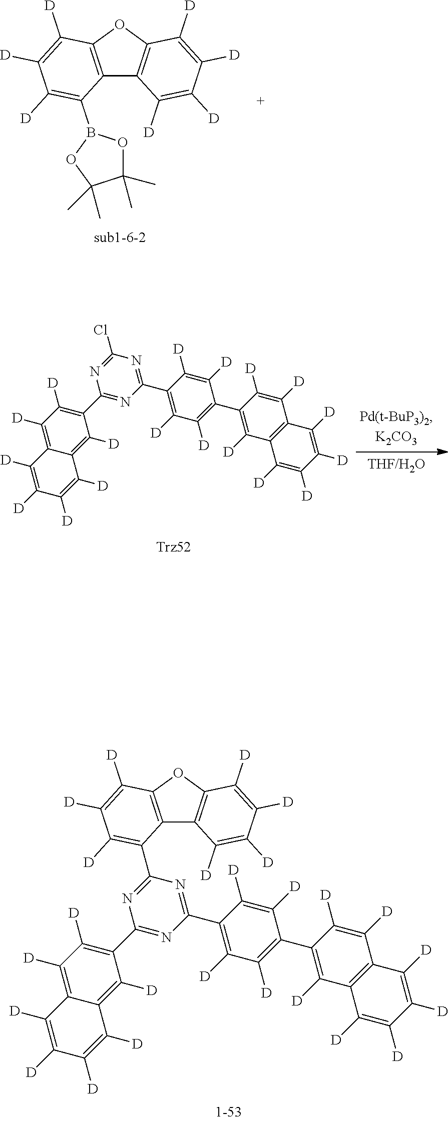 [SMILES: [2H]c1c([2H])c([2H])c2c(oc3c([2H])c([2H])c([2H])c(B4OC(C)(C)C(C)(C)O4)c32)c1[2H] ; [2H]c1c([2H])c(-c2c([2H])c([2H])c3c([2H])c([2H])c([2H])c([2H])c3c2[2H])c([2H])c([2H])c1-c1nc(Cl)nc(-c2c([2H])c([2H])c3c([2H])c([2H])c([2H])c([2H])c3c2[2H])n1 ; [2H]c1c([2H])c(-c2c([2H])c([2H])c3c([2H])c([2H])c([2H])c([2H])c3c2[2H])c([2H])c([2H])c1-c1nc(-c2c([2H])c([2H])c3c([2H])c([2H])c([2H])c([2H])c3c2[2H])nc(-c2c([2H])c([2H])c([2H])c3oc4c([2H])c([2H])c([2H])c([2H])c4c23)n1]

Compound sub1-6-2 (15 g, 49.8 mmol) and Compound Trz52 (24.2 g, 52.3 mmol) were added to 300 ml of THF, and the mixture was stirred and refluxed. Then, potassium carbonate (20.6 g, 149.4 mmol) was dissolved in 62 ml of water and added thereto, and the mixture was sufficiently stirred and then bis(tri-tert-butylphosphine)palladium(0) (0.3 g, 0.5 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.4 g of Compound 1-53. (Yield: 75%, MS: [M+H]⁺=601)

### Synthesis Example 1-54

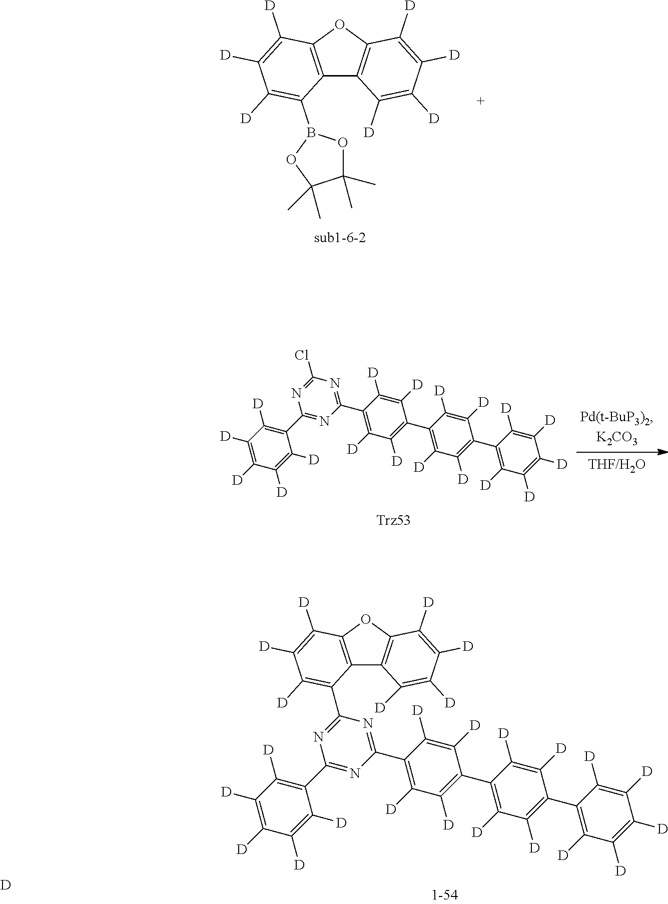

Compound sub1-6-2 (15 g, 49.8 mmol) and Compound Trz53 (22.9 g, 52.3 mmol) were added to 300 ml of THF, and the mixture was stirred and refluxed. Then, potassium carbonate (20.6 g, 149.4 mmol) was dissolved in 62 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 18.7 g of Compound 1-54. (Yield: 65%, MS: [M+H]⁺=577)

### Synthesis Example 1-55

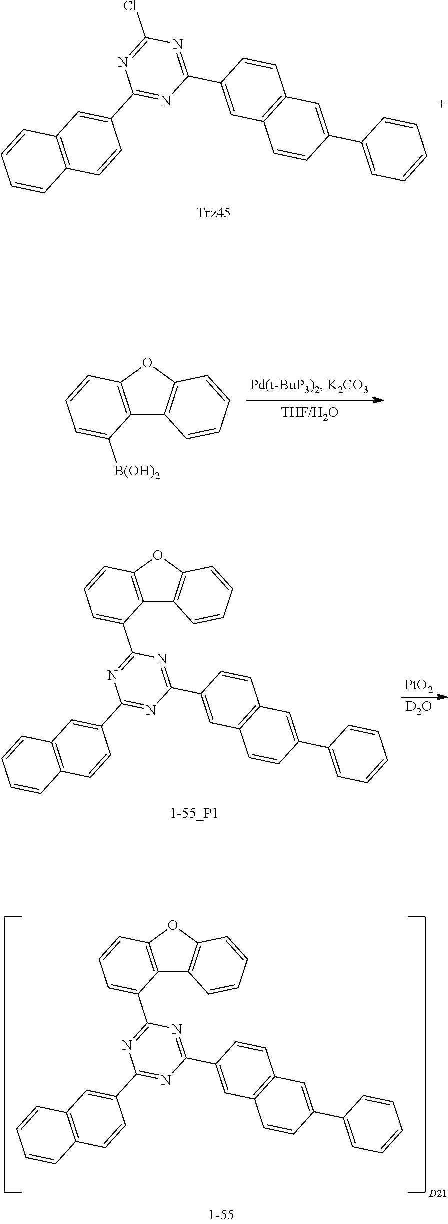

Compound Trz45 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.8 g of Compound 1-55_P1. (Yield: 66%, MS: [M+H]⁺=576)

Compound 1-55_P1 (10 g, 17.4 mmol), PtO₂ (1.2 g, 5.2 mmol), and D₂O (87 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 4.1 g of Compound 1-55. (Yield: 40%, MS[M+H]⁺=598)

### Synthesis Example 1-56

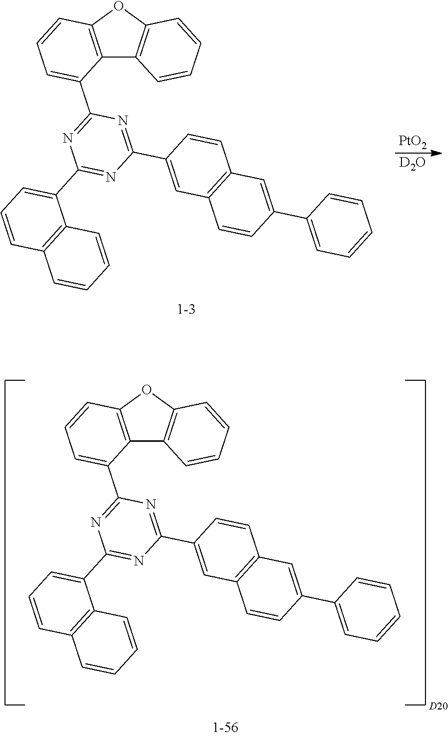

Compound 1-3 (10 g, 17.4 mmol), PtO₂ (1.2 g, 5.2 mmol), and D₂O (87 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 4.4 g of Compound 1-56. (Yield: 43%, MS: [M+H]⁺=597)

### Synthesis Example 1-57

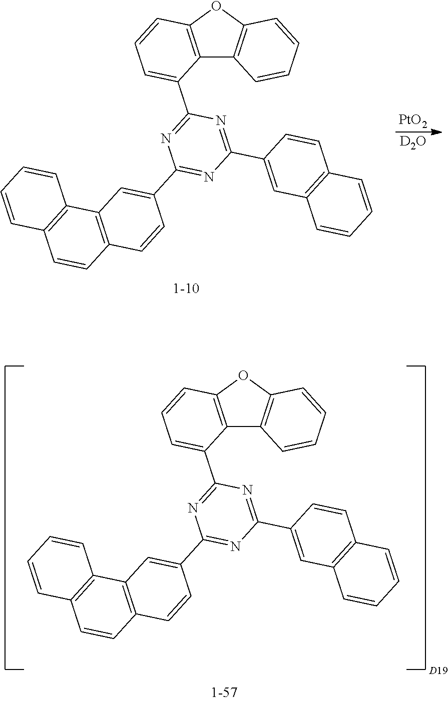

Compound 1-10 (10 g, 18.2 mmol), PtO₂ (1.2 g, 5.5 mmol), and D₂O (91 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 4.1 g of Compound 1-57. (Yield: 40%, MS: [M+H]⁺=570)

### Synthesis Example 1-58

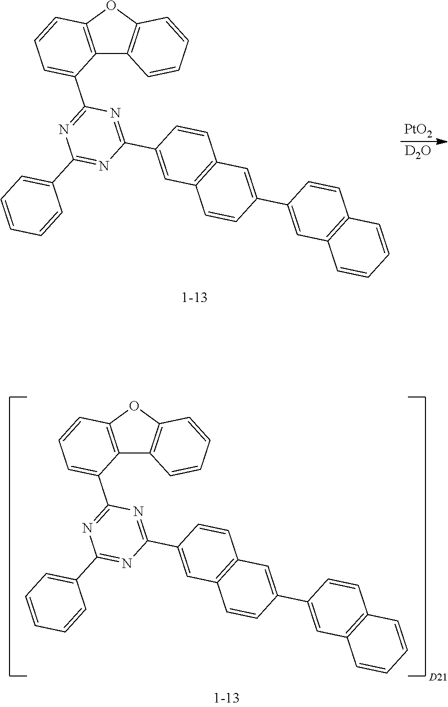

Compound 1-13 (10 g, 17.4 mmol), PtO₂ (1.2 g, 5.2 mmol), and D₂O (87 mi) were added to a shaker tube; and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 4.5 g of Compound 1-58. (Yield: 43%, MS: [M+H]⁺=598)

### Synthesis Example 1-59

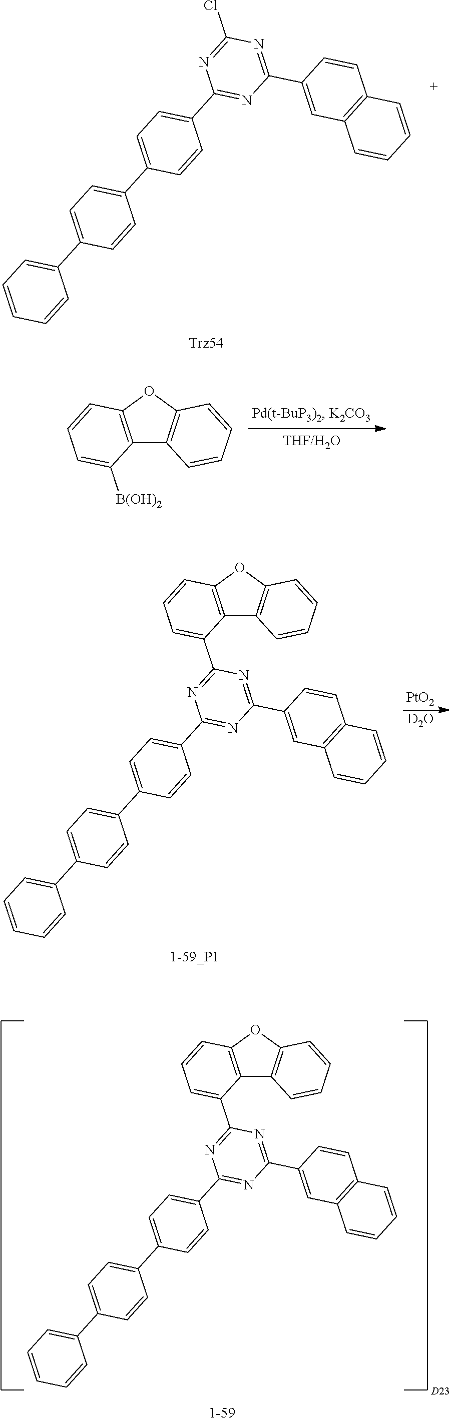

Compound Trz54 (15 g, 31.9 mmol) and dibenzo[b,d]furan-1-ylboronic acid (7.1 g, 33.5 mmol) were added to 300 ml of THF, and the mixture was stirred and refluxed. Then, potassium carbonate (13.2 g, 95.8 mmol) was dissolved in 40 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.2 g of Compound 1-59_P1. (Yield: 74%, MS: [M+H]⁺=602) Compound 1-59_P1 (10 g, 16.6 mmol), PtO₂ (1.1 g, 5 mmol), and D₂O (83 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 4.5 g of Compound 1-59. (Yield: 43%, MS: [M+H]⁺=626)

### Synthesis Example 1-60

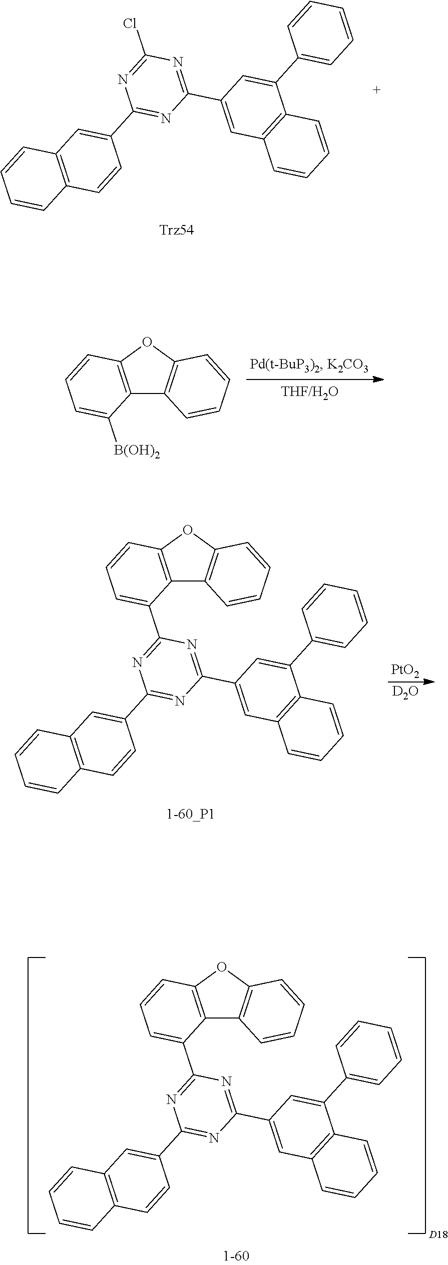

Compound Trz55 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.2 g of Compound 1-60_P1. (Yield: 68%, MS: [M+H]⁺=576)

Compound 1-60_P1 (10 g, 17.4 mmol), PtO₂ (1.2 g, 5.2 mmol), and D₂O (87 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 5.2 g of Compound 1-60. (Yield: 50%, MS: [M+H]⁺=595)

### Synthesis Example 1-61

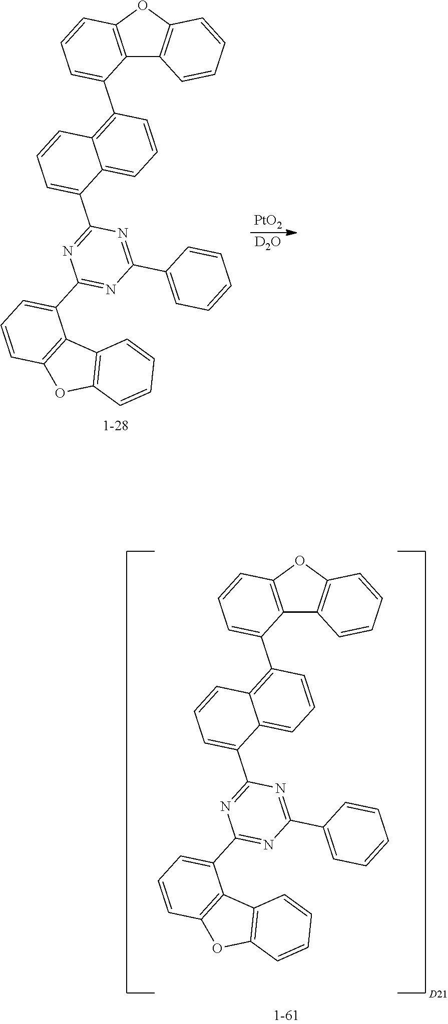

Compound 1-28 (10 g, 16.2 mmol), PtO₂ (1.1 g, 4.9 mmol), and D₂O (81 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 5 g of Compound 1-61. (Yield: 48%, MS: [M+H]⁺=638)

### Synthesis Example 2-1

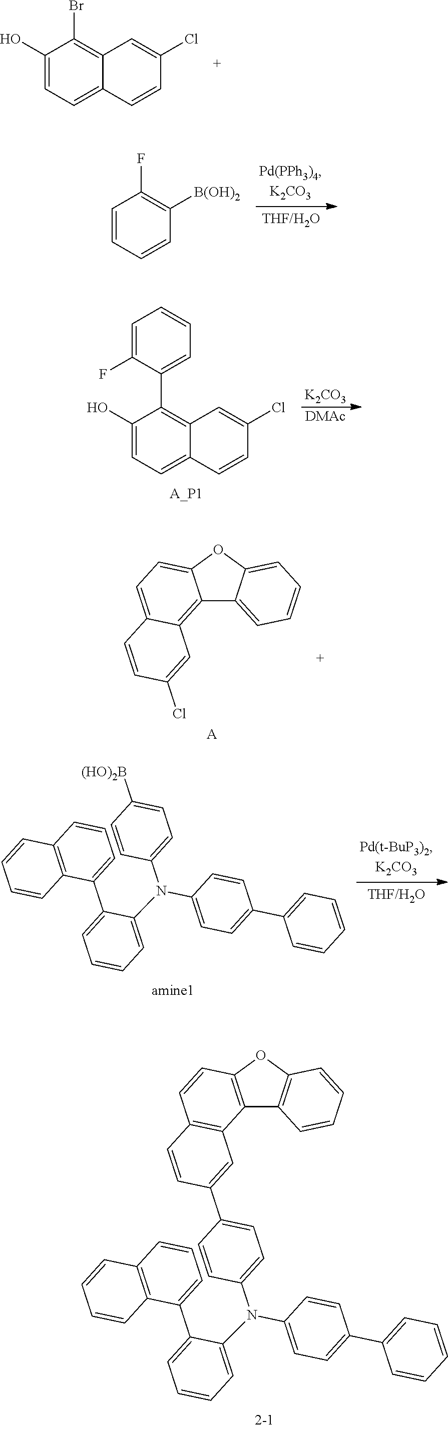

1-Bromo-7-chloronaphthalen-2-ol (15 g, 58.3 mmol) and (2-fluorophenyl)boronic acid (8.6 g, 61.2 mmol) were added to 300 ml of THF, and the mixture was stirred and refluxed. Then, potassium carbonate (24.2 g, 174.8 mmol) was dissolved in water (72 mL), added thereto, stirred sufficiently, and then tetrakis(triphenylphosphine)palladium(0) (0.7 g, 0.6 mmol) was added. After reacting for 6 hours, the reaction mixture was cooled to room temperature was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.4 g of Compound A_P1. (Yield: 78%, MS: [M+H]⁺=273)

Compound A_P1 (15 g, 55 mmol) and potassium carbonate (22.8 g, 165 mmol) were added to 150 ml of DMAc, and the mixture was stirred and refluxed. After reacting for 5 hours, the reaction mixture was cooled to room temperature, poured into 300 ml of water, solidified and filtered to obtain a solid. This was again dissolved in chloroform, washed twice with water, the organic layer was separated, anhydrous magnesium sulfate was added, stirred, filtered, and the filtrate was distilled under reduced pressure. The concentrated compound was purified by silica gel column chromatography to prepare 8.5 g of Compound A. (Yield 61%, MS: [M+H]⁺=253)

Compound A (15 g, 59.4 mmol) and Compound amine1 (30.6 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.6 g of Compound 2-1. (Yield: 70%, MS: [M+H]⁺=664)

### Synthesis Example 2-2

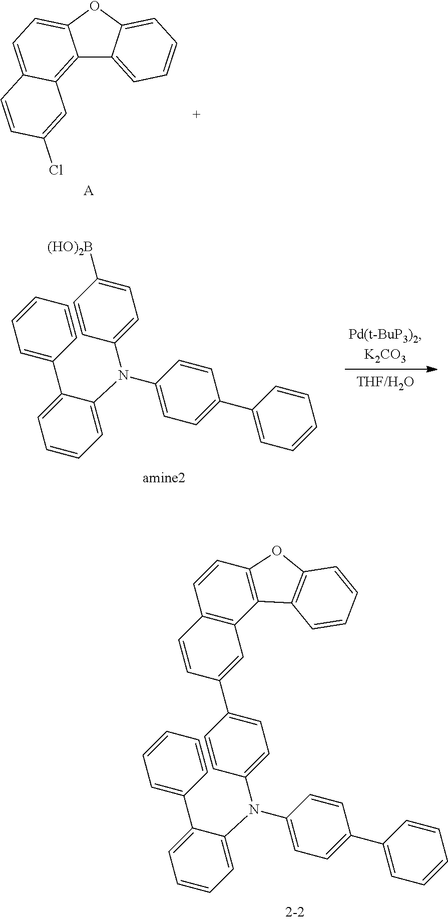

Compound A (15 g, 59.4 mmol) and Compound amine2 (27.5 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.2 g of Compound 2-2. (Yield: 72%, MS: [M+H]⁺=614)

### Synthesis Example 2-3

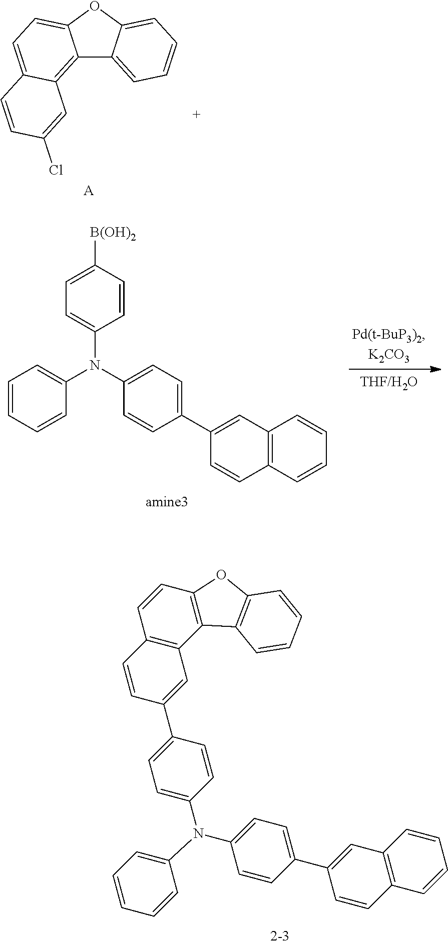

Compound A (15 g, 59.4 mmol) and Compound amine3 (25.9 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.4 g of Compound 2-3. (Yield: 67%, MS: [M+H]⁺=588)

### Synthesis Example 2-4

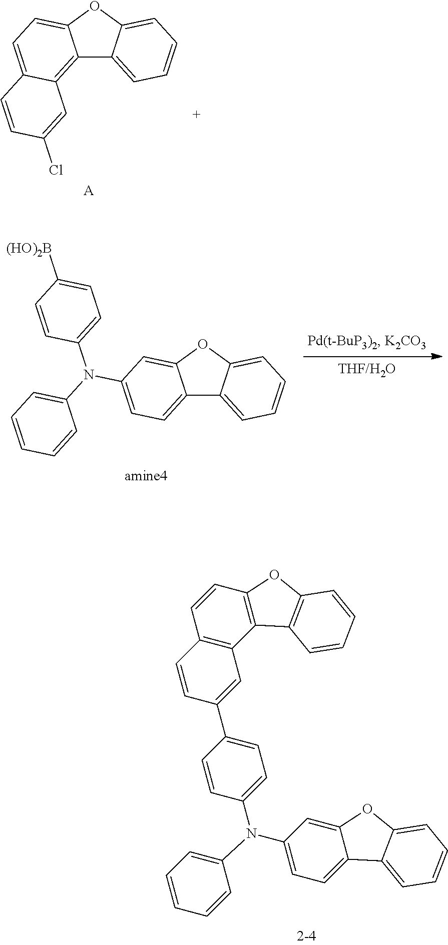

Compound A (15 g, 59.4 mmol) and Compound amine4 (23.6 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.2 g of Compound 2-4. (Yield: 74%, MS: [M+H]⁺=552)

### Synthesis Example 2-5

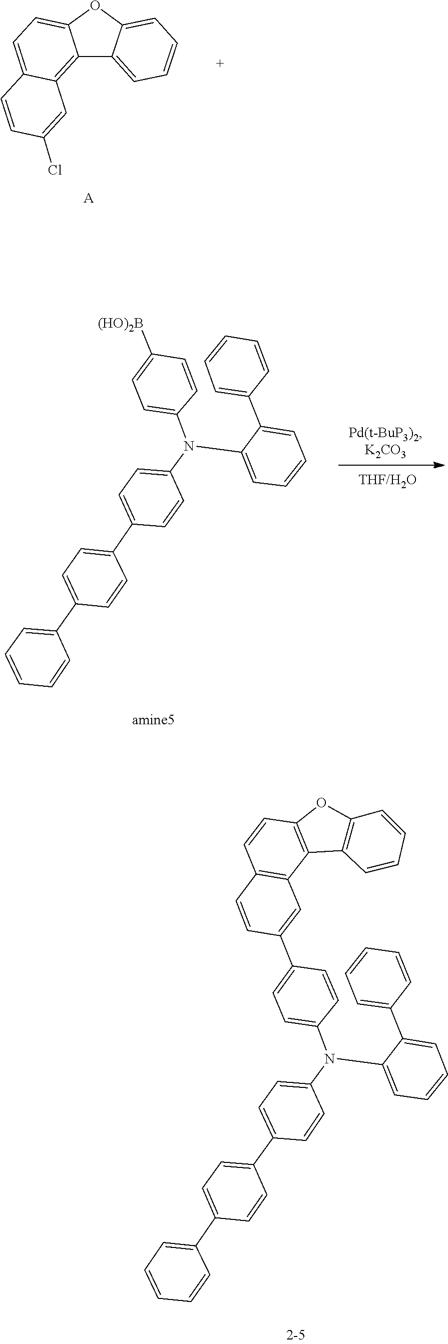

Compound A (15 g, 59.4 mmol) and Compound amine5 (32.3 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.6 g of Compound 2-5. (Yield: 65%, MS: [M+H]⁺=690)

### Synthesis Example 2-6

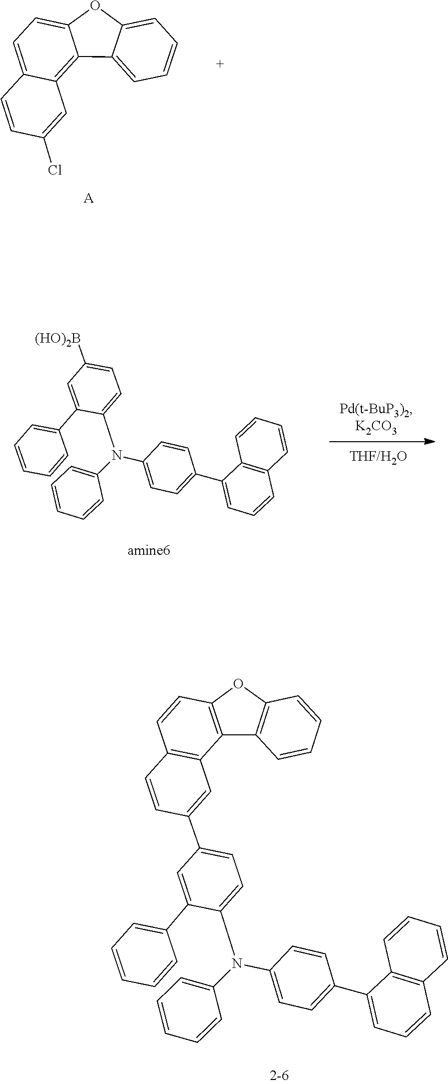

Compound A (15 g, 59.4 mmol) and Compound amine6 (30.6 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 29.1 g of Compound 2-6. (Yield: 74%, MS: [M+H]⁺=664)

### Synthesis Example 2-7

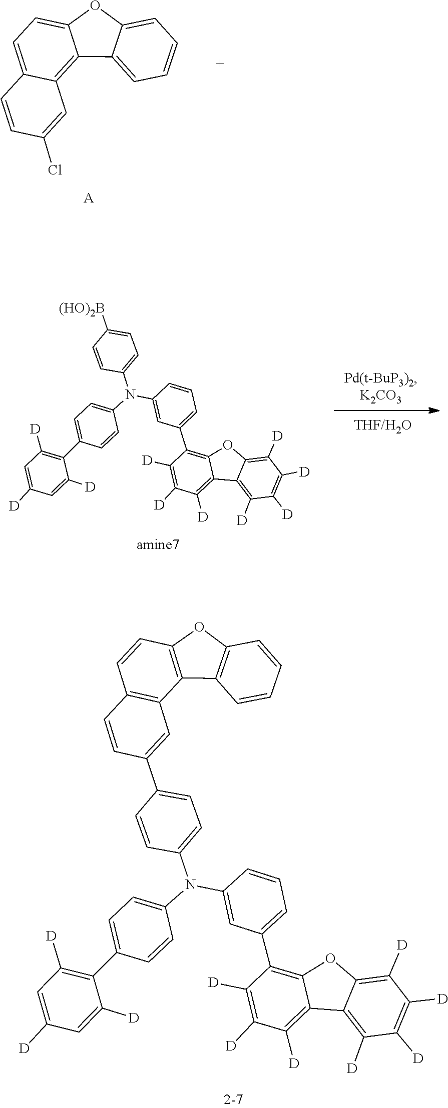

Compound A (15 g, 59.4 mmol) and Compound amine7 (33.7 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.9 g of Compound 2-7. (Yield: 66%, MS: [M+H]⁺=714)

### Synthesis Example 2-8

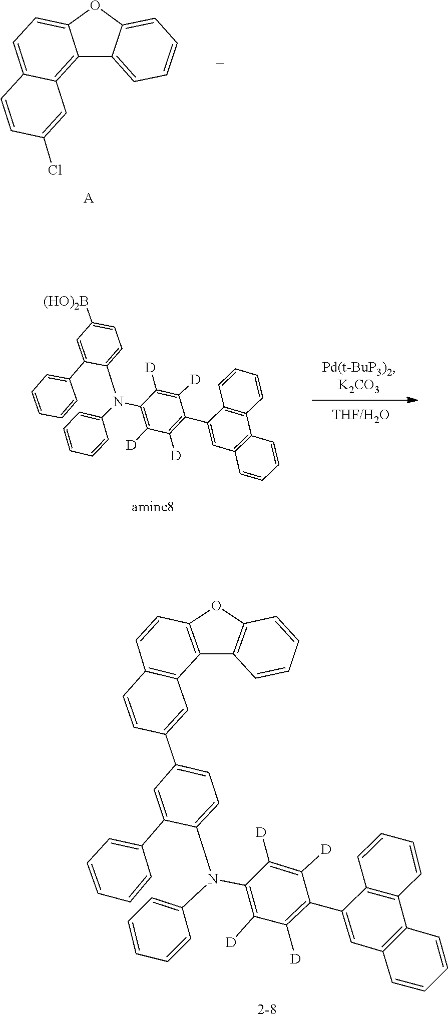

Compound A (15 g, 59.4 mmol) and Compound amine8 (34 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 30.7 g of Compound 2-8. (Yield: 72%, MS: [M+H]⁺=718)

### Synthesis Example 2-9

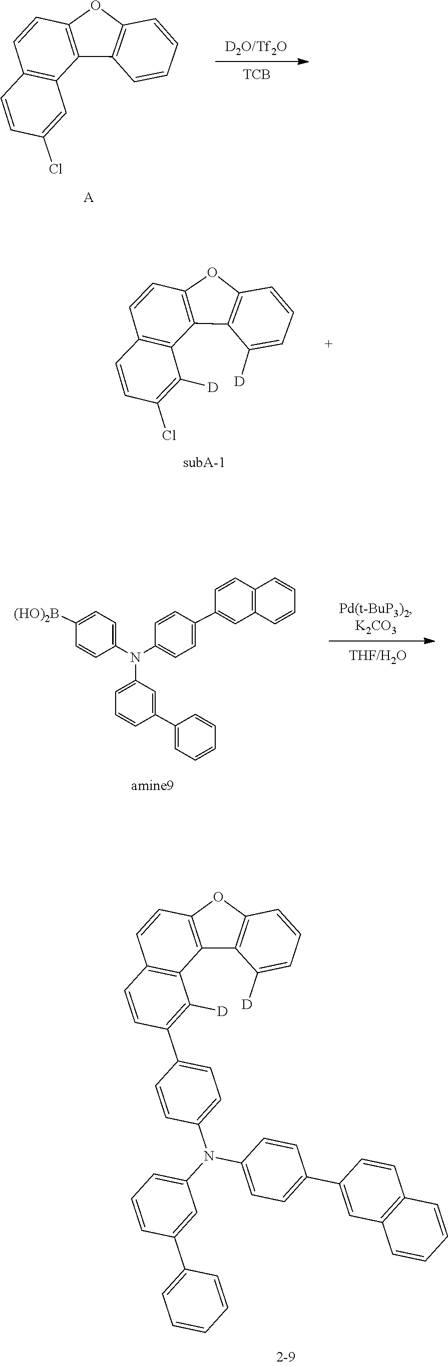

Trifluoromethanesulfonic anhydride (33.5 g, 118.7 mmol) and deuterium oxide (11.9 g, 593.6 mmol) were added at 0° C. and stirred for 5 hours to prepare a solution. Compound A (15 g, 59.4 mmol)) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of Compound A and 1,2,4-trichlorobenzene, and the mixture was stirred while heating to 140° C. and then keeping that temperature. After the reaction for 3 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.4 g of compound subA-1. (Yield: 36%, MS: [M+H]⁺=255)

Compound subA-1 (15 g, 59.6 mmol) and Compound amine9 (30.7 g, 62.6 mmol) were added to 300 ml of THF, and the mixture was stirred and refluxed. Then, potassium carbonate (24.7 g, 178.8 mmol) was dissolved in 74 ml of water and added thereto, and the mixture was sufficiently stirred and then bis(tri-tert-butylphosphine)palladium(0) (0.3 g, 0.6 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 28.9 g of Compound 2-9. (Yield: 73%, MS: [M+H]⁺=666)

### Synthesis Example 2-10

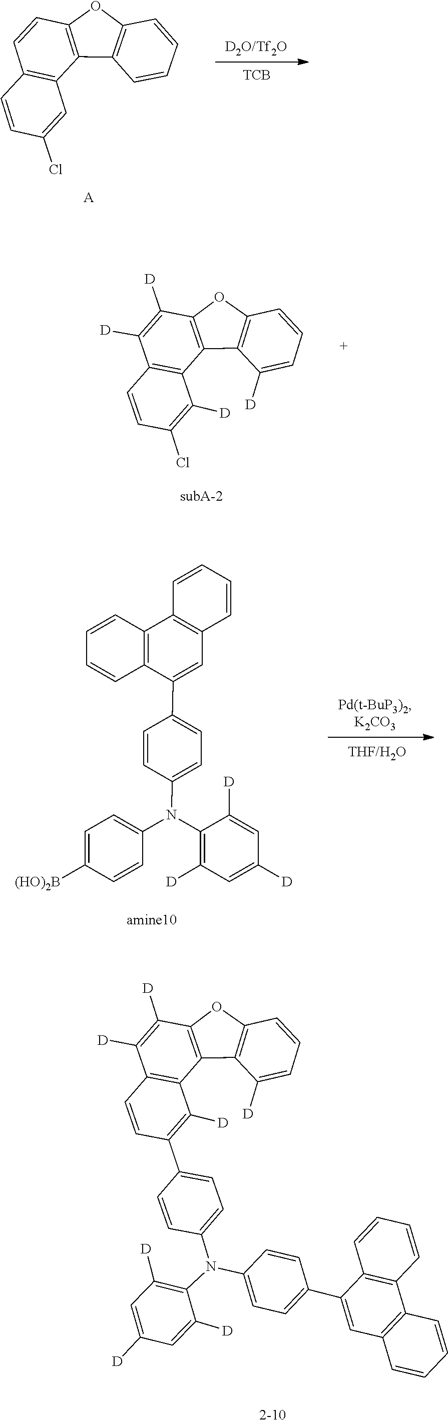

Trifluoromethanesulfonic anhydride (67 g, 237.4 mmol) and deuterium oxide (23.8 g, 1187.2 mmol) were added at 0° C. and stirred for 6 hours to prepare a solution. Compound A (15 g, 59.4 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of Compound A and 1,2,4-trichlorobenzene, and the mixture was stirred while heating to 140° C. and then keeping that temperature. After reacting for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.2 g of compound subA-2. (Yield: 41%, MS: [M+H]⁺=258)

Compound subA-2 (15 g, 58.9 mmol) and Compound amine10 (29 g, 61.8 mmol) were added to 300 ml of THF, and the mixture was stirred and refluxed. Then, potassium carbonate (24.4 g, 176.7 mmol) was dissolved in 73 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.4 g of Compound 2-10. (Yield: 67%, MS: [M+H]⁺=645)

### Synthesis Example 2-11

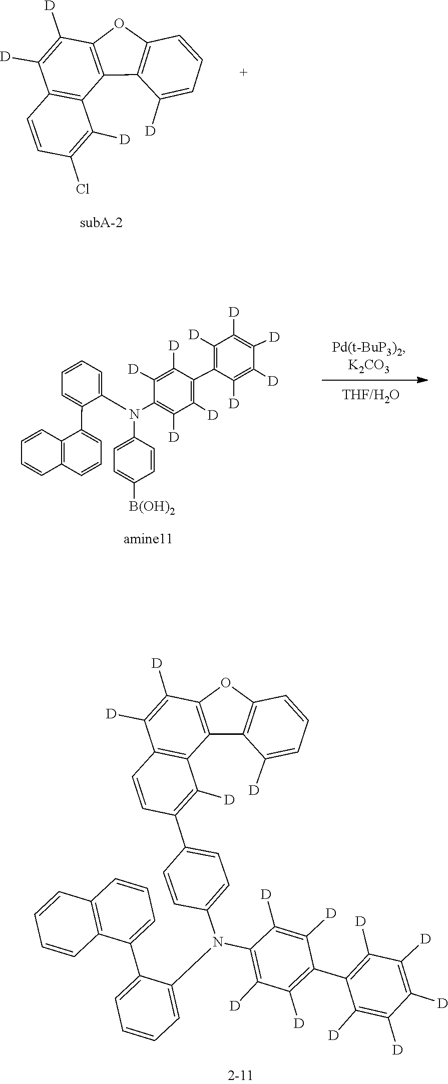 [SMILES: [2H]c1c([2H])c2oc3cccc([2H])c3c2c2c([2H])c(Cl)ccc12 ; [2H]c1c([2H])c([2H])c(-c2c([2H])c([2H])c(N(c3ccc(B(O)O)cc3)c3ccccc3-c3cccc4ccccc34)c([2H])c2[2H])c([2H])c1[2H] ; [2H]c1c([2H])c([2H])c(-c2c([2H])c([2H])c(N(c3ccc(-c4ccc5c([2H])c([2H])c6oc7cccc([2H])c7c6c5c4[2H])cc3)c3ccccc3-c3cccc4ccccc34)c([2H])c2[2H])c([2H])c1[2H]]

Compound subA-2 (15 g, 58.9 mmol) and Compound amine11 (30.9 g, 61.8 mmol) were added to 300 ml of THE, and the mixture was stirred and refluxed. Then, potassium carbonate (24.4 g, 176.7 mmol) was dissolved in 73 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.3 g of Compound 2-11. (Yield: 66%, MS: [M+H]⁺=677)

### Synthesis Example 2-12

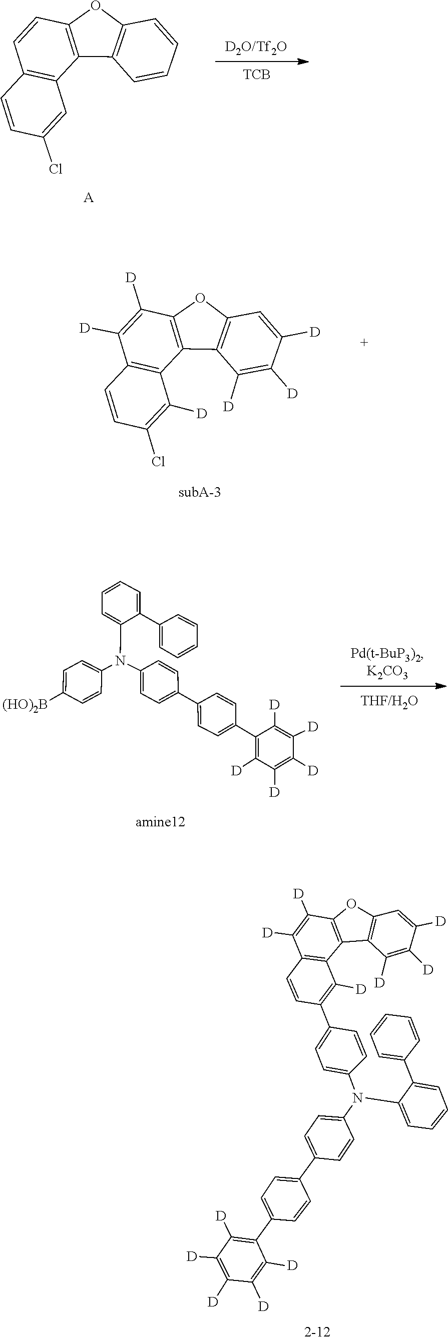

Trifluoromethanesulfonic anhydride (83.7 g, 296.8 mmol) and deuterium oxide (29.7 g, 1484 mmol) were added at 0° C. and stirred for 6 hours to prepare a solution. Compound A (15 g, 59.4 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of Compound A and 1,2,4-trichlorobenzene, and the mixture was stirred while heating to 140° C. and then keeping that temperature. After reacting for 14 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.9 g of compound subA-3. (Yield: 45%, MS: [M+H]⁺=259)

Compound subA-3 (15 g, 58 mmol) and Compound amine12 (31.8 g, 60.9 mmol) were added to 300 ml of THF, and the mixture was stirred and refluxed. Then, potassium carbonate (24 g, 173.9 mmol) was dissolved in 72 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.4 g of Compound 2-12. (Yield: 65%, MS: [M+H]⁺=701)

### Synthesis Example 2-13

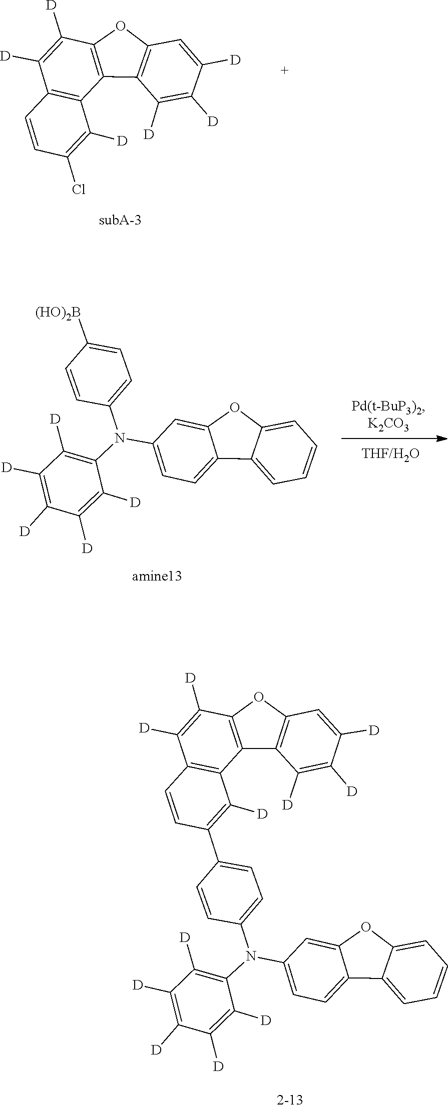 [SMILES: [2H]c1cc2oc3c([2H])c([2H])c4ccc(Cl)c([2H])c4c3c2c([2H])c1[2H] ; [2H]c1c([2H])c([2H])c(N(c2ccc(B(O)O)cc2)c2ccc3c(c2)oc2ccccc23)c([2H])c1[2H] ; [2H]c1cc2oc3c([2H])c([2H])c4ccc(-c5ccc(N(c6ccc7c(c6)oc6ccccc67)c6c([2H])c([2H])c([2H])c([2H])c6[2H])cc5)c([2H])c4c3c2c([2H])c1[2H]]

Compound subA-3 (15 g, 58 mmol) and Compound amine13 (23.4 g, 60.9 mmol) were added to 300 ml of THF, and the mixture was stirred and refluxed. Then, potassium carbonate (24 g, 173.9 mmol) was dissolved in 72 ml of water and added thereto, and the mixture was sufficiently stirred and then bis(tri-tert-butylphosphine)palladium(0) (0.3 g, 0.6 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.5 g of Compound 2-13. (Yield: 66%, MS: [M+H]⁺=563)

### Synthesis Example 2-14

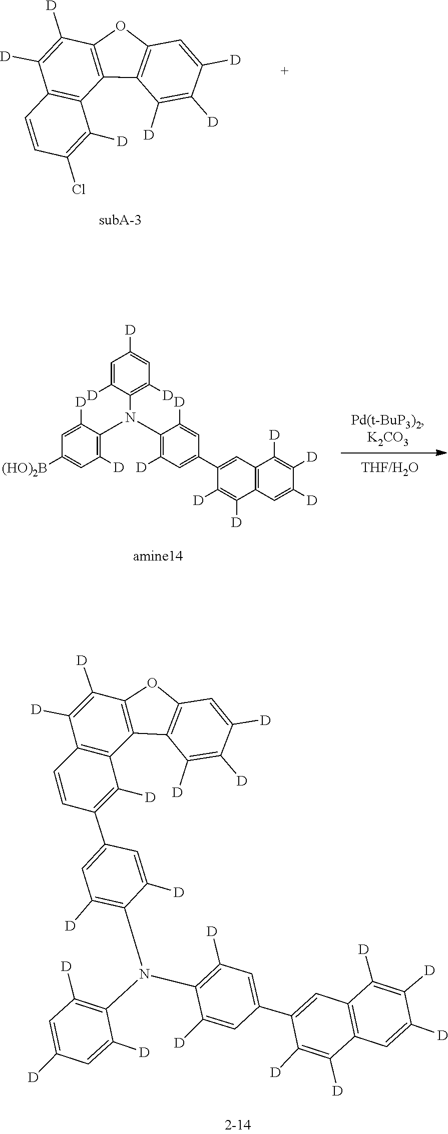 [SMILES: [2H]c1cc2oc3c([2H])c([2H])c4ccc(Cl)c([2H])c4c3c2c([2H])c1[2H] ; [2H]c1cc([2H])c(N(c2c([2H])cc(B(O)O)cc2[2H])c2c([2H])cc(-c3cc4c([2H])c([2H])c([2H])cc4c([2H])c3[2H])cc2[2H])c([2H])c1 ; [2H]c1cc([2H])c(N(c2c([2H])cc(-c3cc4c([2H])c([2H])c([2H])cc4c([2H])c3[2H])cc2[2H])c2c([2H])cc(-c3ccc4c([2H])c([2H])c5oc6cc([2H])c([2H])c([2H])c6c5c4c3[2H])cc2[2H])c([2H])c1]

Compound subA-3 (15 g, 58 mmol) and Compound amine14 (26 g, 60.9 mmol) were added to 300 ml of THF, and the mixture was stirred and refluxed. Then, potassium carbonate (24 g, 173.9 mmol) was dissolved in 72 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.3 g of Compound 2-14. (Yield: 72%, MS: [M+H]⁺=606)

### Synthesis Example 2-15

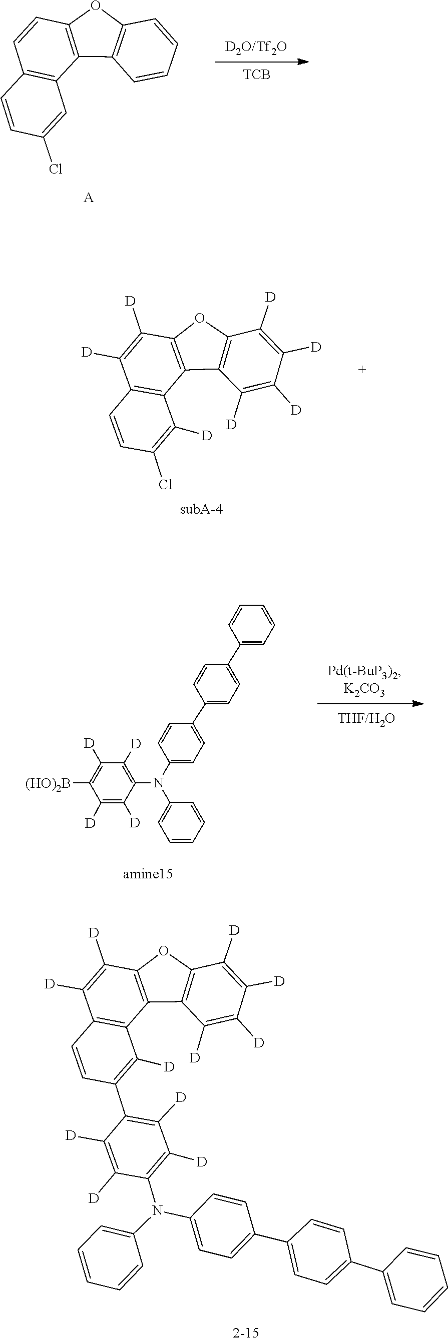

Trifluoromethanesulfonic anhydride (117.2 g, 415.5 mmol) and deuterium oxide (41.6 g, 2077.6 mmol) were added at 0° C. and stirred for 6 hours to prepare a solution. Compound A (15 g, 59.4 mmol)) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of Compound A and 1,2,4-trichlorobenzene, and the mixture was stirred while heating to 140° C. and then keeping that temperature. After reacting for 20 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.8 g of compound subA-4. (Yield: 38%, MS: [M+H]⁺=260)

Compound subA-4 (15 g, 57.8 mmol) and Compound amine15 (27 g, 60.6 mmol) were added to 300 ml of THF, and the mixture was stirred and refluxed. Then, potassium carbonate (23.9 g, 173.3 mmol) was dissolved in 72 ml of water and added thereto, and the mixture was sufficiently stirred and then bis(tri-tert-butylphosphine)palladium(0) (0.3 g, 0.6 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.8 g of Compound 2-15. (Yield: 66%, MS: [M+H]⁺=625)

### Synthesis Example 2-16

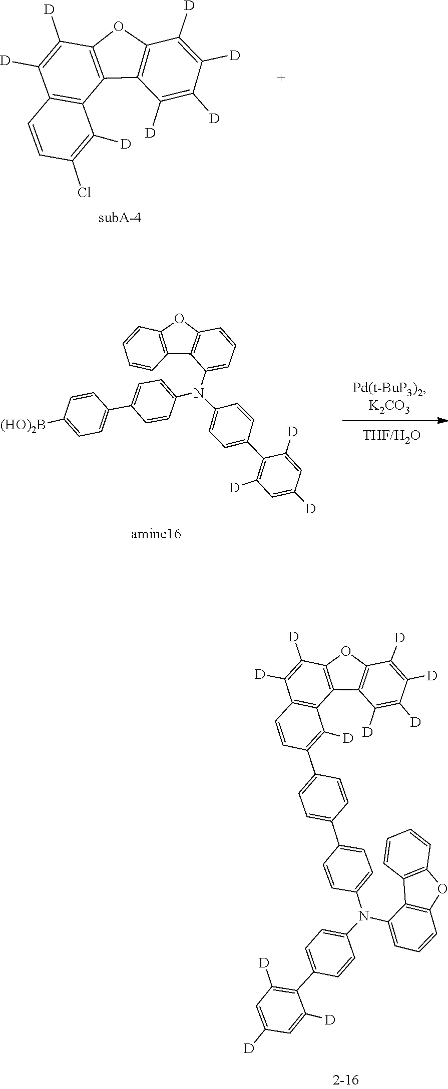 [SMILES: [2H]c1c([2H])c([2H])c2c(oc3c([2H])c([2H])c4ccc(Cl)c([2H])c4c32)c1[2H] ; [2H]c1cc([2H])c(-c2ccc(N(c3ccc(-c4ccc(B(O)O)cc4)cc3)c3cccc4oc5ccccc5c34)cc2)c([2H])c1 ; [2H]c1cc([2H])c(-c2ccc(N(c3ccc(-c4ccc(-c5ccc6c([2H])c([2H])c7oc8c([2H])c([2H])c([2H])c([2H])c8c7c6c5[2H])cc4)cc3)c3cccc4oc5ccccc5c34)cc2)c([2H])c1]

Compound subA-4 (15 g, 57.8 mmol) and Compound amine16 (32.4 g, 60.6 mmol) were added to 300 ml of THF, and the mixture was stirred and refluxed. Then, potassium carbonate (23.9 g, 173.3 mmol) was dissolved in 72 ml of water and added thereto, and the mixture was sufficiently stirred and then bis(tri-tert-butylphosphine)palladium(0) (0.3 g, 0.6 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.8 g of Compound 2-16. (Yield: 65%, MS: [M+H]⁺=714)

### Synthesis Example 2-17

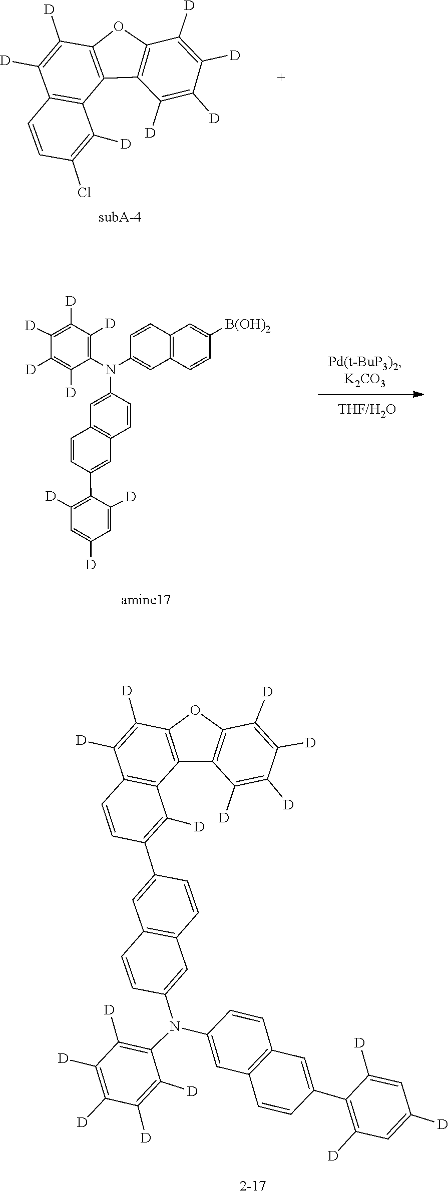 [SMILES: [2H]c1c([2H])c([2H])c2c(oc3c([2H])c([2H])c4ccc(Cl)c([2H])c4c32)c1[2H] ; [2H]c1cc([2H])c(-c2ccc3cc(N(c4ccc5cc(B(O)O)ccc5c4)c4c([2H])c([2H])c([2H])c([2H])c4[2H])ccc3c2)c([2H])c1 ; [2H]c1cc([2H])c(-c2ccc3cc(N(c4ccc5cc(-c6ccc7c([2H])c([2H])c8oc9c([2H])c([2H])c([2H])c([2H])c9c8c7c6[2H])ccc5c4)c4c([2H])c([2H])c([2H])c([2H])c4[2H])ccc3c2)c([2H])c1]

Compound subA-4 (15 g, 57.8 mmol) and Compound amine17 (28.7 g, 60.6 mmol) were added to 300 ml of THF, and the mixture was stirred and refluxed. Then, potassium carbonate (23.9 g, 173.3 mmol) was dissolved in 72 ml of water and added thereto, and the mixture was sufficiently stirred and then bis(tri-tert-butylphosphine)palladium(0) (0.3 g, 0.6 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.9 g of Compound 2-17. (Yield: 66%, MS: [M+H]⁺=653)

### Synthesis Example 2-18

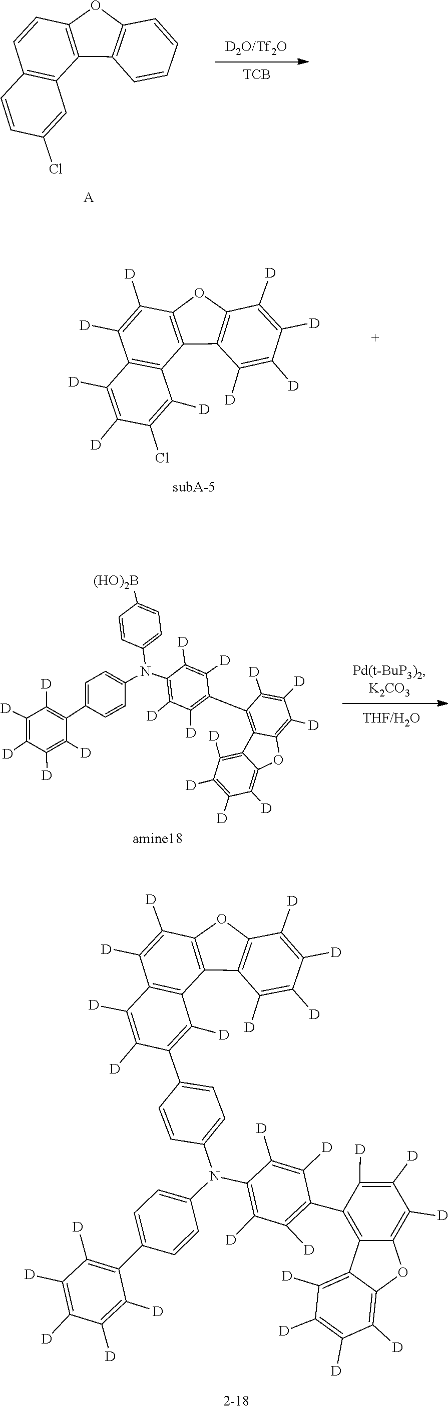

Trifluoromethanesulfonic anhydride (150.7 g, 534.2 mmol) and deuterium oxide (53.5 g, 2671.2 mmol) were added at 0° C. and stirred for 6 hours to prepare a solution. Compound A (15 g, 59.4 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of Compound A and 1,2,4-trichlorobenzene, and the mixture was stirred while heating to 140° C. and then keeping that temperature. After reacting for 28 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.5 g of Compound subA-5. (Yield: 42%, MS: [M+H]⁺=262)

Compound subA-5 (15 g, 57.3 mmol) and Compound amine18 (32.9 g, 60.2 mmol) were added to 300 ml of THF, and the mixture was stirred and refluxed. Then, potassium carbonate (23.8 g, 171.9 mmol) was dissolved in 71 ml of water and added thereto, and the mixture was sufficiently stirred and then bis(tri-tert-butylphosphine)palladium(0) (0.3 g, 0.6 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 31.3 g of Compound 2-18. (Yield: 75%, MS: [M+H]⁺=729)

### Synthesis Example 2-19

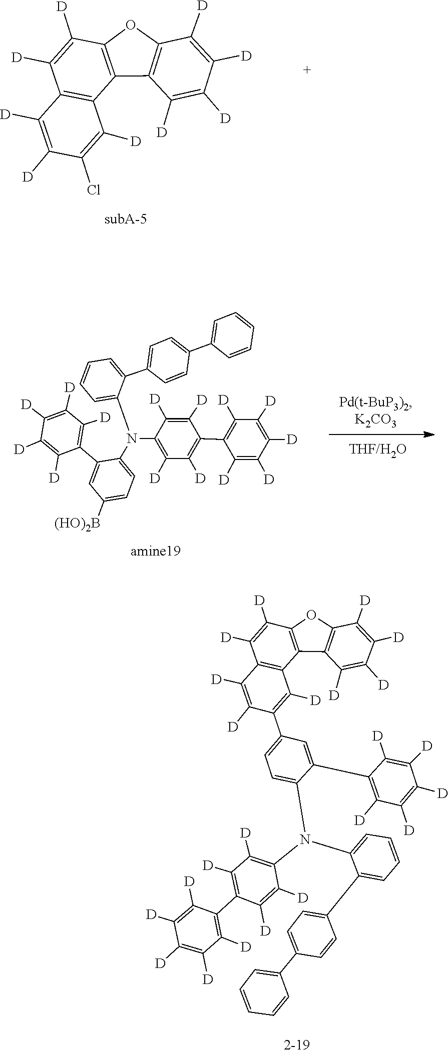 [SMILES: [2H]c1c([2H])c([2H])c2c(oc3c([2H])c([2H])c4c([2H])c([2H])c(Cl)c([2H])c4c32)c1[2H] ; [2H]c1c([2H])c([2H])c(-c2cc(B(O)O)ccc2N(c2ccccc2-c2ccc(-c3ccccc3)cc2)c2c([2H])c([2H])c(-c3c([2H])c([2H])c([2H])c([2H])c3[2H])c([2H])c2[2H])c([2H])c1[2H] ; [2H]c1c([2H])c([2H])c(-c2cc(-c3c([2H])c([2H])c4c([2H])c([2H])c5oc6c([2H])c([2H])c([2H])c([2H])c6c5c4c3[2H])ccc2N(c2ccccc2-c2ccc(-c3ccccc3)cc2)c2c([2H])c([2H])c(-c3c([2H])c([2H])c([2H])c([2H])c3[2H])c([2H])c2[2H])c([2H])c1[2H]]

Compound subA-5 (15 g, 57.3 mmol) and Compound amine19 (36.6 g, 60.2 mmol) were added to 300 ml of THF, and the mixture was stirred and refluxed. Then, potassium carbonate (23.8 g, 171.9 mmol) was dissolved in 71 ml of water and added thereto, and the mixture was sufficiently stirred and then bis(tri-tert-butylphosphine)palladium(0) (0.3 g, 0.6 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 30.3 g of Compound 2-19. (Yield: 67%, MS: [M+H]⁺=789)

### Synthesis Example 2-20

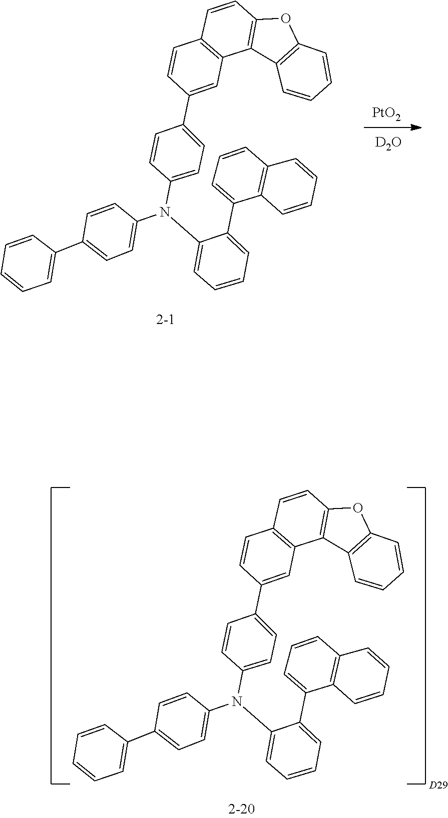

Compound 2-1 (10 g, 15.1 mmol), PtO₂ (1 g, 4.5 mmol) and D₂O (75 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 3.2 g of Compound 2-20. (Yield: 31%, MS: [M+H]⁺=694)

### Synthesis Example 2-21

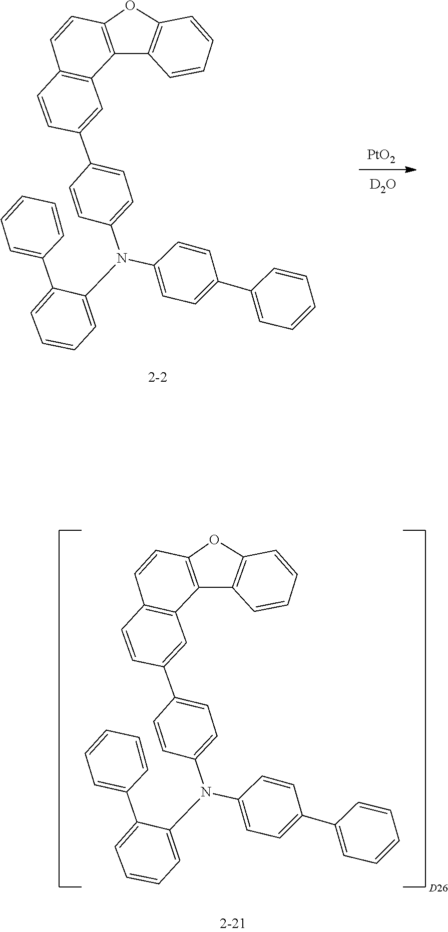

Compound 2-2 (10 g, 16.3 mmol), PtO₂ (1.1 g, 4.9 mmol), and D₂O (81 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 4.7 g of Compound 2-21. (Yield: 45%, MS: [M+H]⁺=641)

### Synthesis Example 2-22

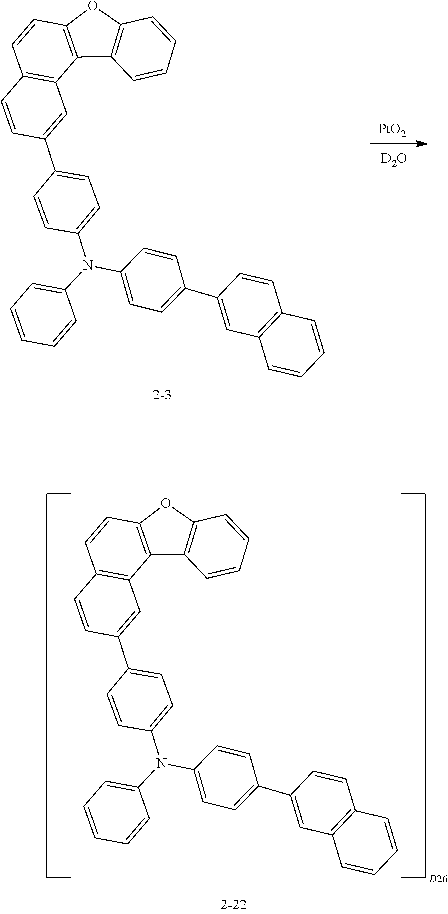

Compound 2-3 (10 g, 17 mmol), PtO₂ (1.2 g, 5.1 mmol) and D₂O (85 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 4.4 g of Compound 2-22. (Yield: 42%, MS: [M+H]⁺=615)

### Synthesis Example 2-23

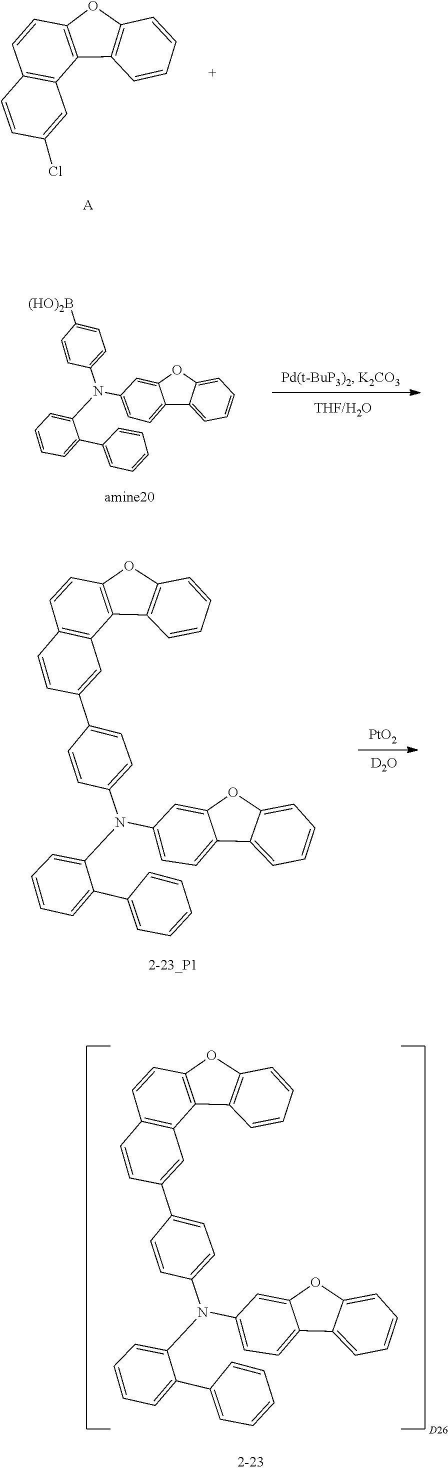

Compound A (15 g, 59.4 mmol) and Compound amine20 (28.4 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.6 g of Compound 2-23_P1. (Yield: 66%, MS: [M+H]⁺=628)

Compound 2-23_P1 (10 g, 15.9 mmol), PtO₂ (1.1 g, 4.8 mmol) and D₂O (80 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 4.4 g of Compound 2-23. (Yield: 42%, MS: [M+H]⁺=655)

### Synthesis Example 2-24

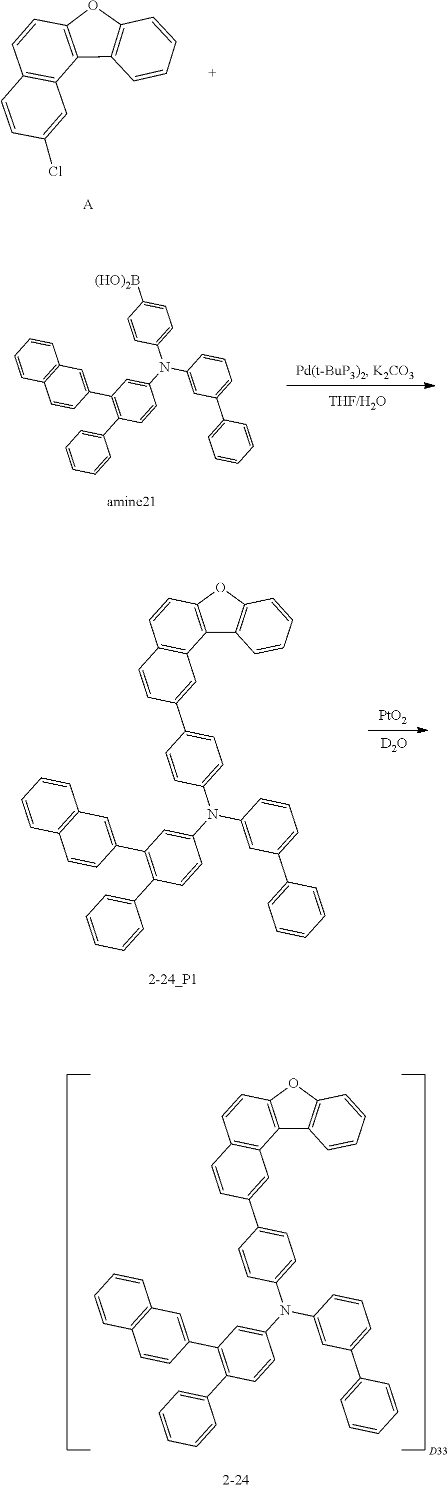

Compound A (15 g, 59.4 mmol) and Compound amine21 (35.4 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 28.5 g of Compound 2-24_P1. (Yield: 65%, MS: [M+H]⁺=740)

Compound 2-24_P1 (10 g, 13.5 mmol), PtO₂ (0.9 g, 4.1 mmol) and D₂O (68 mi) were added to a shaker tube; and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 4.4 g of Compound 2-24. (Yield: 42%, MS: [M+H]⁺=774)

### Synthesis Example 2-25

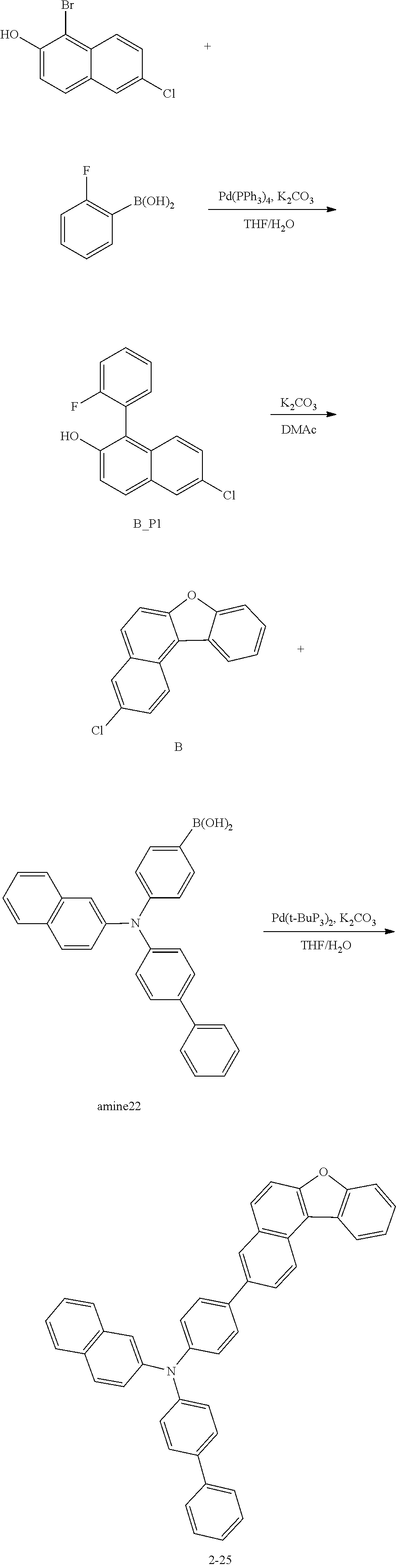

1-Bromo-6-chloronaphthalen-2-ol (15 g, 58.3 mmol) and (2-fluorophenyl)boronic acid (8.6 g, 61.2 mmol) were added to 300 ml of THF, and the mixture was stirred and refluxed. Then, potassium carbonate (24.2 g, 174.8 mmol) was dissolved in 72 ml of water and added thereto, and the mixture was sufficiently stirred and then tetrakis(triphenylphosphine)palladium(0) (0.7 g, 0.6 mmol) was added. After reacting for 6 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 10.5 g of Compound B_P1. (Yield: 66%, MS: [M+H]⁺=273)

Compound B_P1 (15 g, 55 mmol) and potassium carbonate (22.8 g, 165 mmol) were added to 150 ml of DMAc, and the mixture was stirred and refluxed. After reacting for 5 hours, the reaction mixture was cooled to room temperature, poured into 300 ml of water, solidified and filtered to obtain a solid. This was again dissolved in chloroform, washed twice with water, the organic layer was separated, anhydrous magnesium sulfate was added, stirred, filtered, and the filtrate was distilled under reduced pressure. The concentrated compound was purified by silica gel column chromatography to prepare 8.5 g of Compound B. (Yield 65%, MS: [M+H]⁺=253)

Compound B (15 g, 59.4 mmol) and Compound amine22 (25.9 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.7 g of Compound 2-25. (Yield: 68%, MS: [M+H]⁺=588)

### Synthesis Example 2-26

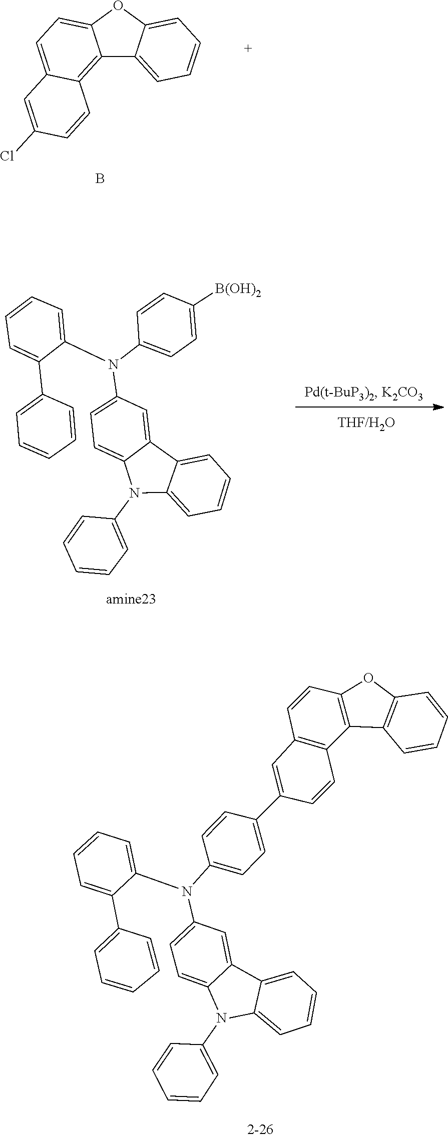

Compound B (15 g, 59.4 mmol) and Compound amine23 (33.1 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.9 g of Compound 2-26. (Yield: 67%, MS: [M+H]⁺=703)

### Synthesis Example 2-27

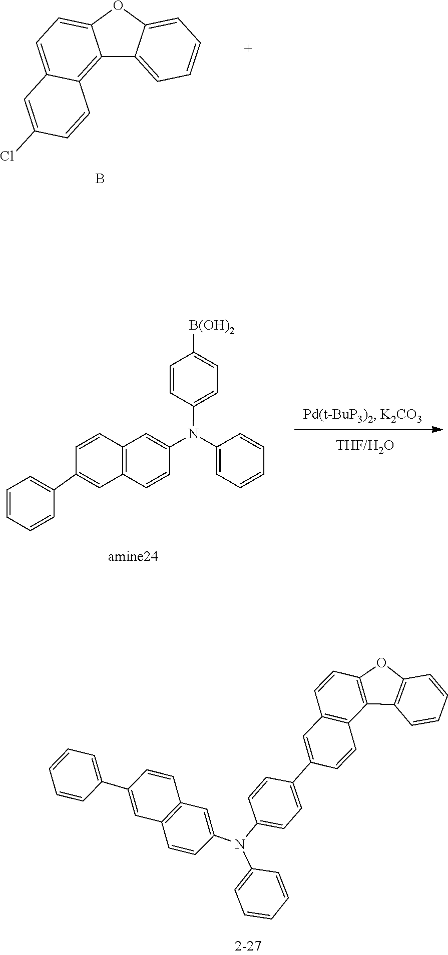

Compound B (15 g, 59.4 mmol) and Compound amine24 (25.9 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.4 g of Compound 2-27. (Yield: 73%, MS: [M+H]⁺=588)

### Synthesis Example 2-28

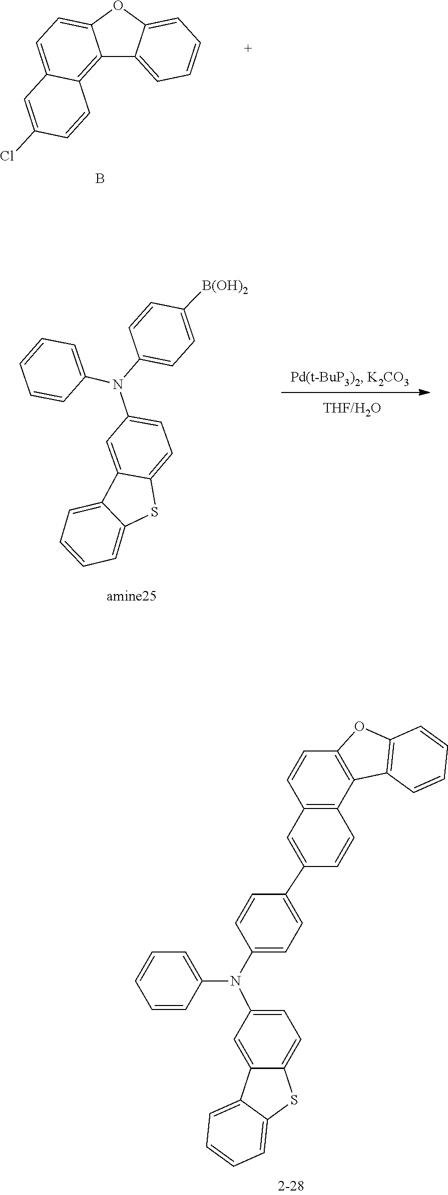

Compound B (15 g, 59.4 mmol) and Compound amine25 (24.6 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.9 g of Compound 2-28. (Yield: 68%, MS: [M+H]⁺=568)

### Synthesis Example 2-29

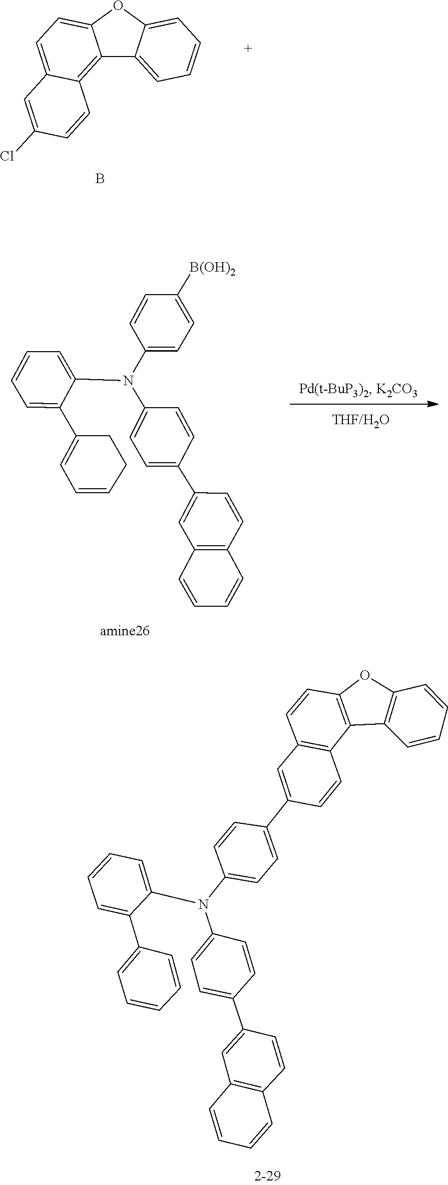

Compound B (15 g, 59.4 mmol) and Compound amine26 (30.6 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.4 g of Compound 2-29. (Yield: 67%, MS: [M+H]⁺=664)

### Synthesis Example 2-30

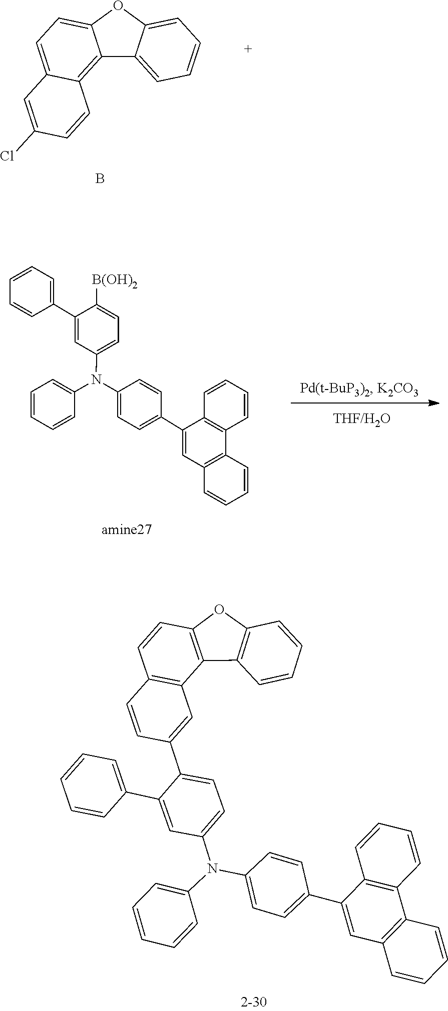

Compound B (15 g, 59.4 mmol) and Compound amine27 (33.7 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 29.6 g of Compound 2-30. (Yield: 70%, MS: [M+H]⁺=714)

### Synthesis Example 2-31

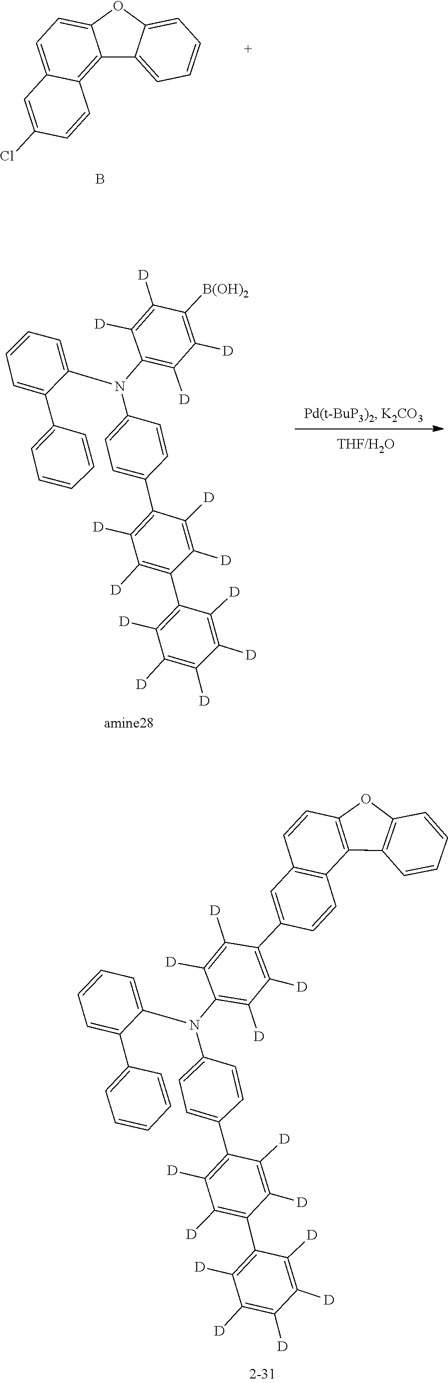

Compound B (15 g, 59.4 mmol) and Compound amine28 (33.1 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.5 g of Compound 2-31. (Yield: 66%, MS: [M+H]⁺=703)

### Synthesis Example 2-32

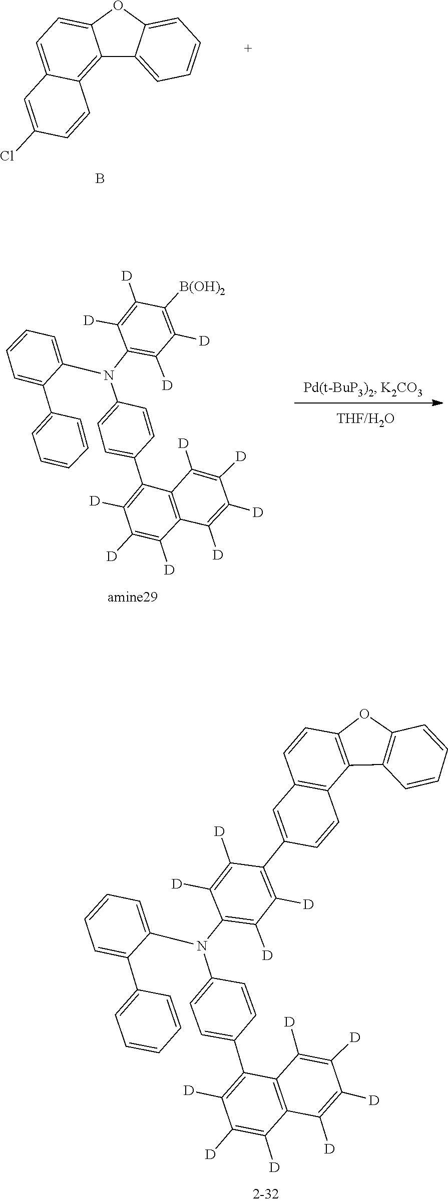

Compound B (15 g, 59.4 mmol) and Compound amine29 (31.3 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26 g of Compound 2-32. (Yield: 65%, MS: [M+H]⁺=675)

### Synthesis Example 2-33

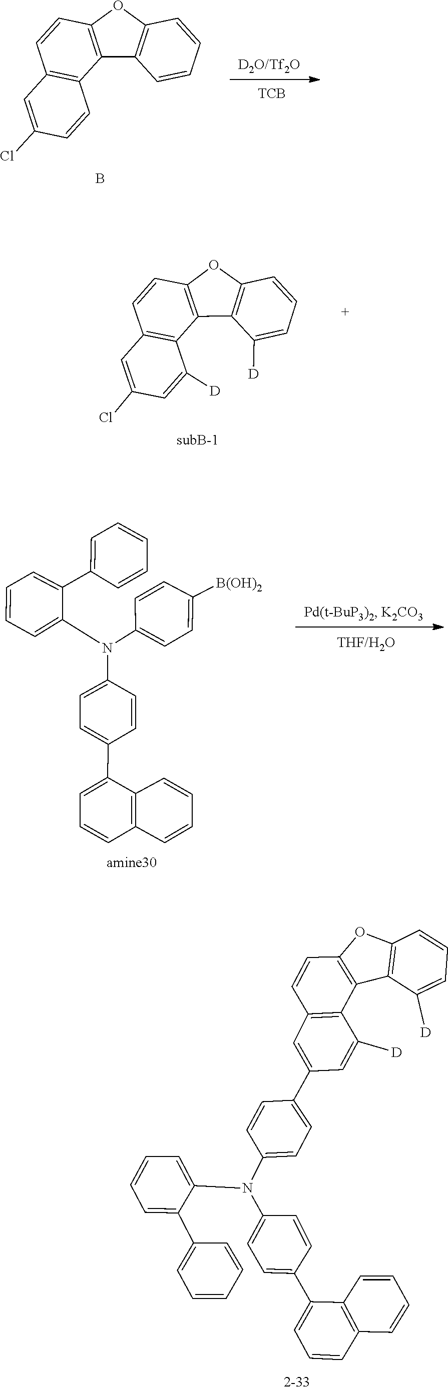

Trifluoromethanesulfonic anhydride (33.5 g, 118.7 mmol) and deuterium oxide (11.9 g, 593.6 mmol) were added at 0° C. and stirred for 5 hours to prepare a solution. Compound B (15 g, 59.4 mmol)) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of Compound A and 1,2,4-trichlorobenzene, and the mixture was stirred while heating to 140° C. and then keeping that temperature. After reacting for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.5 g of compound subB-1. (Yield: 43%, MS: [M+H]⁺=255)

Compound subB-1 (15 g, 58.9 mmol) and Compound amine30 (30.4 g, 61.8 mmol) were added to 300 ml of THF, and the mixture was stirred and refluxed. Then, potassium carbonate (24.4 g, 176.7 mmol) was dissolved in 73 ml of water and added thereto, and the mixture was sufficiently stirred and then bis(tri-tert-butylphosphine)palladium(0) (0.3 g, 0.6 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.8 g of Compound 2-33. (Yield: 71%, MS: [M+H]⁺=666)

### Synthesis Example 2-34

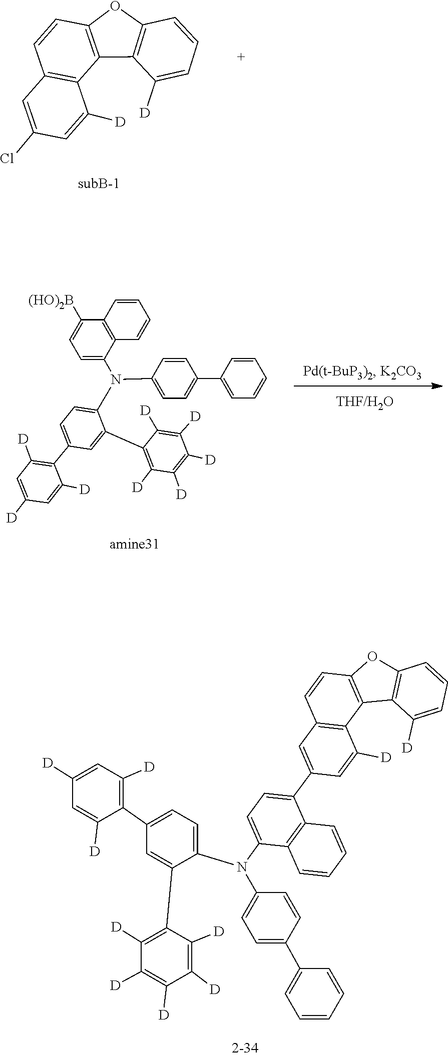 [SMILES: [2H]c1cc(Cl)cc2ccc3oc4cccc([2H])c4c3c12 ; [2H]c1cc([2H])c(-c2ccc(N(c3ccc(-c4ccccc4)cc3)c3ccc(B(O)O)c4ccccc34)c(-c3c([2H])c([2H])c([2H])c([2H])c3[2H])c2)c([2H])c1 ; [2H]c1cc([2H])c(-c2ccc(N(c3ccc(-c4ccccc4)cc3)c3ccc(-c4cc([2H])c5c(ccc6oc7cccc([2H])c7c65)c4)c4ccccc34)c(-c3c([2H])c([2H])c([2H])c([2H])c3[2H])c2)c([2H])c1]

Compound subB-1 (15 g, 58.9 mmol) and Compound amine31 (35.6 g, 61.8 mmol) were added to 300 ml of THF, and the mixture was stirred and refluxed. Then, potassium carbonate (24.4 g, 176.7 mmol) was dissolved in 73 ml of water and added thereto, and the mixture was sufficiently stirred and then bis(tri-tert-butylphosphine)palladium(0) (0.3 g, 0.6 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 33.1 g of Compound 2-34. (Yield: 75%, MS: [M+H]⁺=750)

### Synthesis Example 2-35

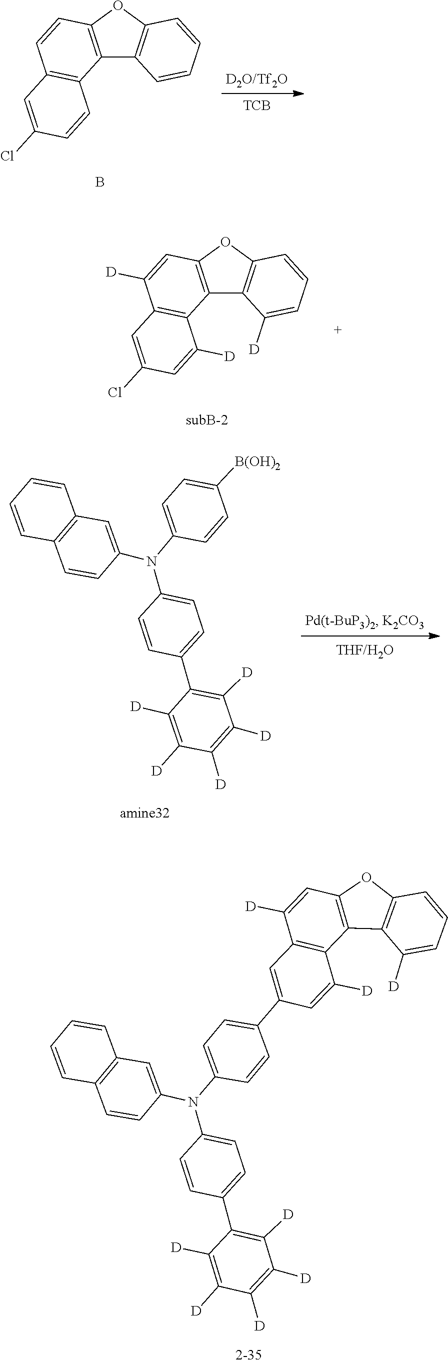

Trifluoromethanesulfonic anhydride (50.2 g, 178.1 mmol) and deuterium oxide (17.8 g, 890.4 mmol) were added at 0° C. and stirred for 6 hours to prepare a solution. Compound B (15 g, 59.4 mmol)) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of Compound A and 1,2,4-trichlorobenzene, and the mixture was stirred while heating to 140° C. and then keeping that temperature. After reacting for 7 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.7 g of compound subB-2. (Yield: 44%, MS: [M+H]⁺=256)

Compound subB-2 (15 g, 58.7 mmol) and Compound amine32 (25.9 g, 61.6 mmol) were added to 300 ml of THF, and the mixture was stirred and refluxed. Then, potassium carbonate (24.3 g, 176 mmol) was dissolved in 73 ml of water and added thereto, and the mixture was sufficiently stirred and then bis(tri-tert-butylphosphine)palladium(0) (0.3 g, 0.6 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.2 g of Compound 2-35. (Yield: 75%, MS: [M+H]⁺=596)

### Synthesis Example 2-36

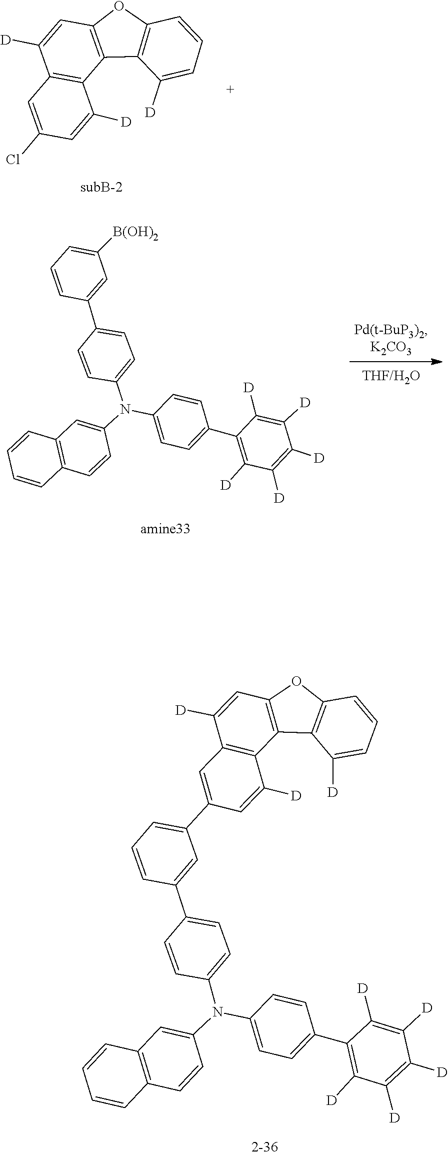 [SMILES: [2H]c1cc2oc3cccc([2H])c3c2c2c([2H])cc(Cl)cc12 ; [2H]c1c([2H])c([2H])c(-c2ccc(N(c3ccc(-c4cccc(B(O)O)c4)cc3)c3ccc4ccccc4c3)cc2)c([2H])c1[2H] ; [2H]c1c([2H])c([2H])c(-c2ccc(N(c3ccc(-c4cccc(-c5cc([2H])c6c(c5)c([2H])cc5oc7cccc([2H])c7c56)c4)cc3)c3ccc4ccccc4c3)cc2)c([2H])c1[2H]]

Compound subB-2 (15 g, 58.7 mmol) and Compound amine33 (30.6 g, 61.6 mmol) were added to 300 ml of THF, and the mixture was stirred and refluxed. Then, potassium carbonate (24.3 g, 176 mmol) was dissolved in 73 ml of water and added thereto, and the mixture was sufficiently stirred and then bis(tri-tert-butylphosphine)palladium(0) (0.3 g, 0.6 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.6 g of Compound 2-36. (Yield: 70%, MS: [M+H]⁺=672)

### Synthesis Example 2-37

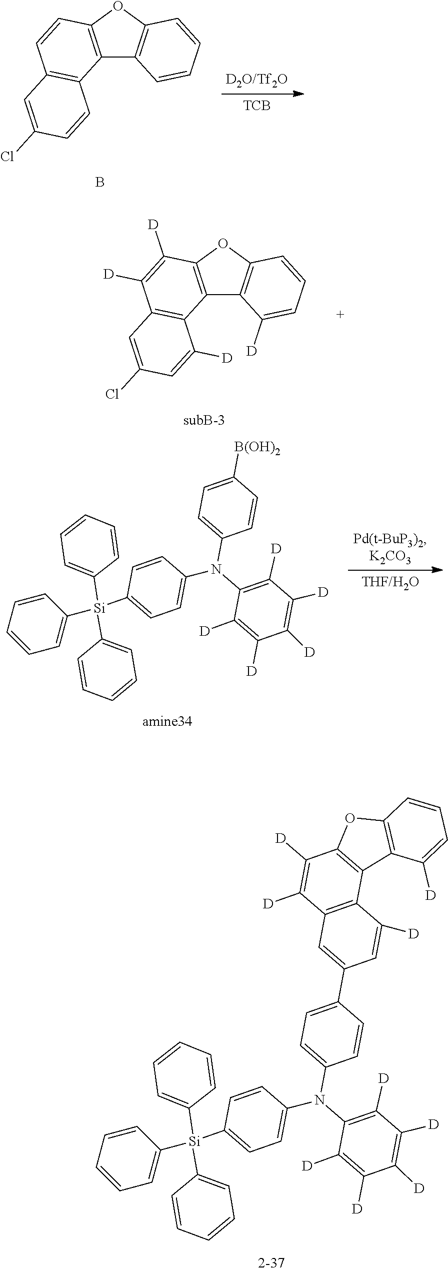

Trifluoromethanesulfonic anhydride (67 g, 237.4 mmol) and deuterium oxide (23.8 g, 1187.2 mmol) were added at 0° C. and stirred for 6 hours to prepare a solution. Compound B (15 g, 59.4 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of Compound B and 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.9 g of compound subB-3. (Yield: 39%, MS: [M+H]⁺=258)

Compound subB-3 (15 g, 58.4 mmol) and Compound amine34 (33.9 g, 61.4 mmol) were added to 300 ml of THF, and the mixture was stirred and refluxed. Then, potassium carbonate (24.2 g, 175.3 mmol) was dissolved in 73 ml of water and added thereto, and the mixture was sufficiently stirred and then bis(tri-tert-butylphosphine)palladium(0) (0.3 g, 0.6 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 30.6 g of Compound 2-37. (Yield: 72%, MS: [M+H]⁺=729)

### Synthesis Example 2-38

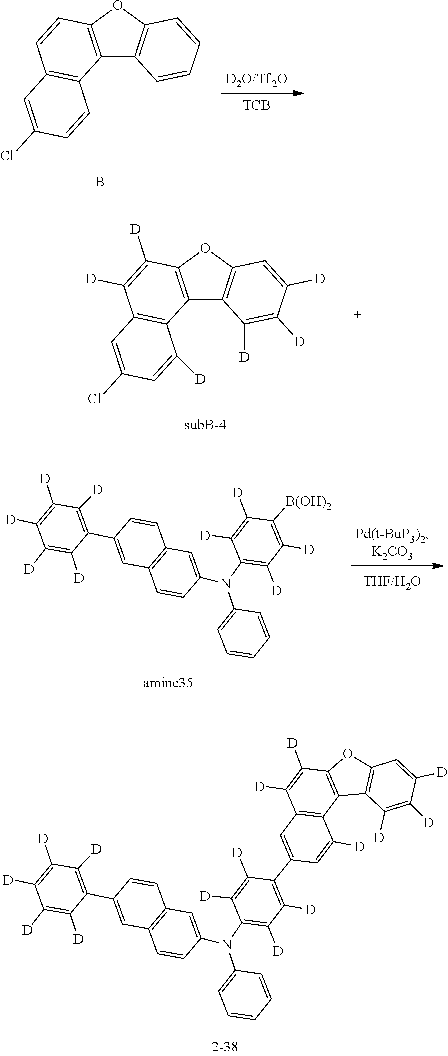

Trifluoromethanesulfonic anhydride (100.5 g, 356.2 mmol) and deuterium oxide (35.7 g, 1780.8 mmol) were added at 0° C. and stirred for 6 hours to prepare a solution. Compound B (15 g, 59.4 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of Compound B and 1,2,4-trichlorobenzene, and the mixture was stirred while heating to 140° C. and then keeping that temperature. After reacting for 17 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.4 g of compound subB-4. (Yield: 35%, MS: [M+H]⁺=259)

Compound subB-4 (15 g, 58 mmol) and Compound amine35 (25.8 g, 60.9 mmol) were added to 300 ml of THF, and the mixture was stirred and refluxed. Then, potassium carbonate (24 g, 173.9 mmol) was dissolved in 72 ml of water and added thereto, and the mixture was sufficiently stirred and then bis(tri-tert-butylphosphine)palladium(0) (0.3 g, 0.6 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.7 g of Compound 2-38. (Yield: 65%, MS: [M+H]⁺=603)

### Synthesis Example 2-39

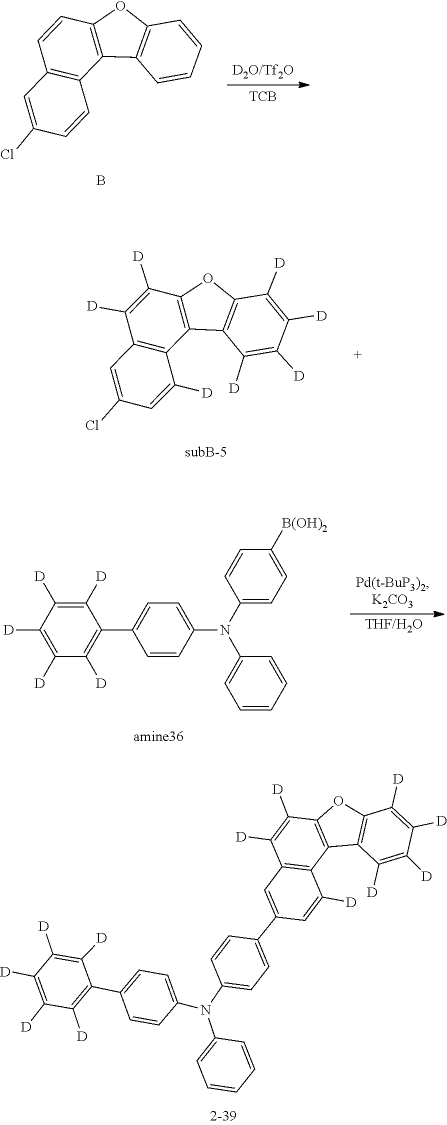

Trifluoromethanesulfonic anhydride (117.2 g, 415.5 mmol) and deuterium oxide (41.6 g, 2077.6 mmol) were added at 0° C. and stirred for 6 hours to prepare a solution. Compound B (15 g, 59.4 mmol)) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of Compound B and 1,2,4-trichlorobenzene, and the mixture was stirred while heating to 140° C. and then keeping that temperature. After reacting for 21 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.7 g of compound subB-5. (Yield: 37%, MS: [M+H]⁺=260)

Compound subB-5 (15 g, 57.8 mmol) and Compound amine36 (22.5 g, 60.6 mmol) were added to 300 ml of THF, and the mixture was stirred and refluxed. Then, potassium carbonate (23.9 g, 173.3 mmol) was dissolved in 72 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.2 g of Compound 2-39. (Yield: 70%, MS: [M+H]⁺=550)

### Synthesis Example 2-40

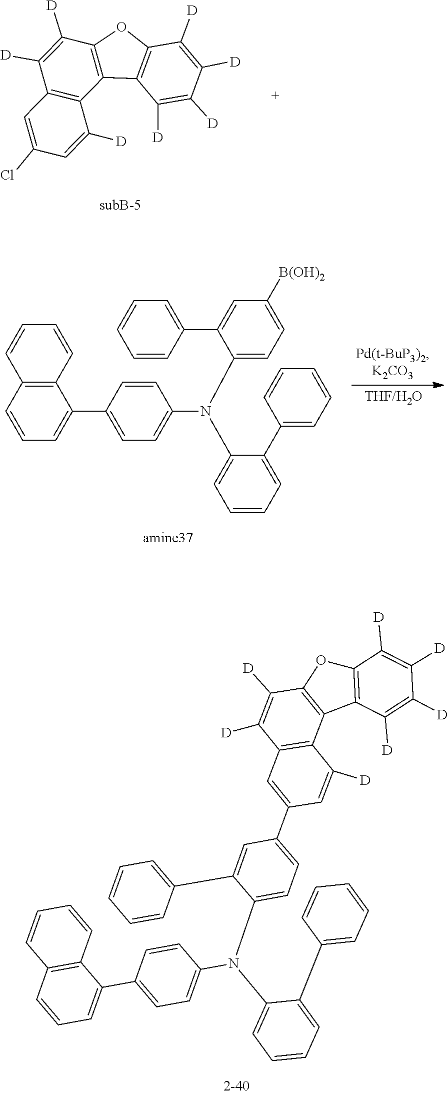 [SMILES: [2H]c1c([2H])c([2H])c2c(oc3c([2H])c([2H])c4cc(Cl)cc([2H])c4c32)c1[2H] ; OB(O)c1ccc(N(c2ccc(-c3cccc4ccccc34)cc2)c2ccccc2-c2ccccc2)c(-c2ccccc2)c1 ; [2H]c1c([2H])c([2H])c2c(oc3c([2H])c([2H])c4cc(-c5ccc(N(c6ccc(-c7cccc8ccccc78)cc6)c6ccccc6-c6ccccc6)c(-c6ccccc6)c5)cc([2H])c4c32)c1[2H]]

Compound subB-5 (15 g, 57.8 mmol) and Compound amine37 (34.4 g, 60.6 mmol) were added to 300 ml of THF, and the mixture was stirred and refluxed. Then, potassium carbonate (23.9 g, 173.3 mmol) was dissolved in 72 ml of water and added thereto, and the mixture was sufficiently stirred and then bis(tri-tert-butylphosphine)palladium(0) (0.3 g, 0.6 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 30.2 g of Compound 2-40. (Yield: 70%, MS: [M+H]⁺=747)

### Synthesis Example 2-41

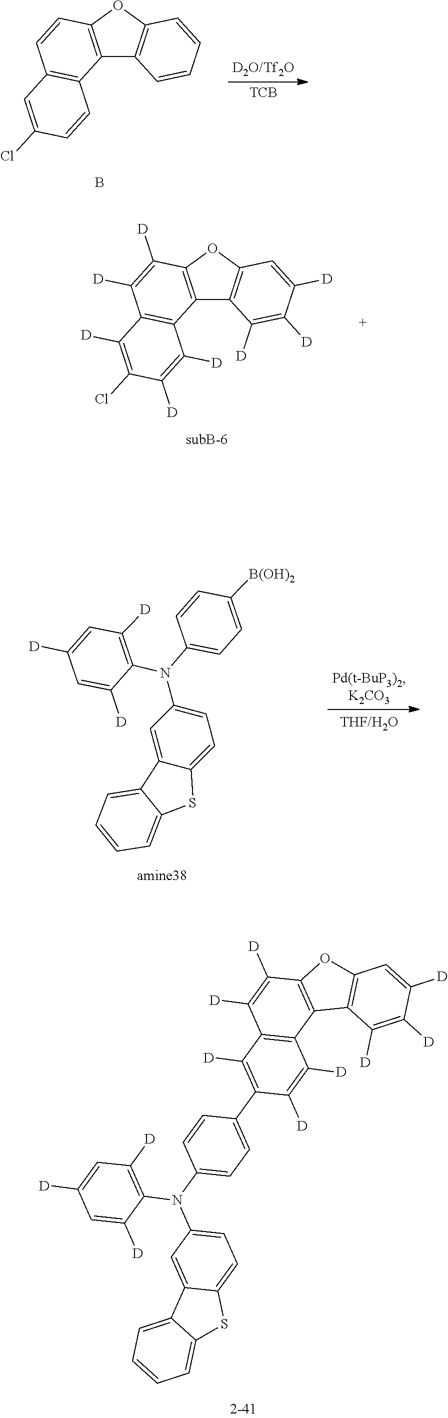

Trifluoromethanesulfonic anhydride (134 g, 474.9 mmol) and deuterium oxide (47.6 g, 2374.4 mmol) were added at 0° C. and stirred for 6 hours to prepare a solution. Compound B (15 g, 59.4 mmol)) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of Compound B and 1,2,4-trichlorobenzene, and the mixture was stirred while heating to 140° C. and then keeping that temperature. After reacting for 25 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.6 g of compound subB-6. (Yield: 43%, MS: [M+H]⁺=261)

Compound subB-6 (15 g, 57.5 mmol) and Compound amine38 (24.1 g, 60.4 mmol) were added to 300 ml of THF, and the mixture was stirred and refluxed. Then, potassium carbonate (23.9 g, 172.6 mmol) was dissolved in 72 ml of water and added thereto, and the mixture was sufficiently stirred and then bis(tri-tert-butylphosphine)palladium(0) (0.3 g, 0.6 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.3 g of Compound 2-41. (Yield: 67%, MS: [M+H]⁺=579)

### Synthesis Example 2-42

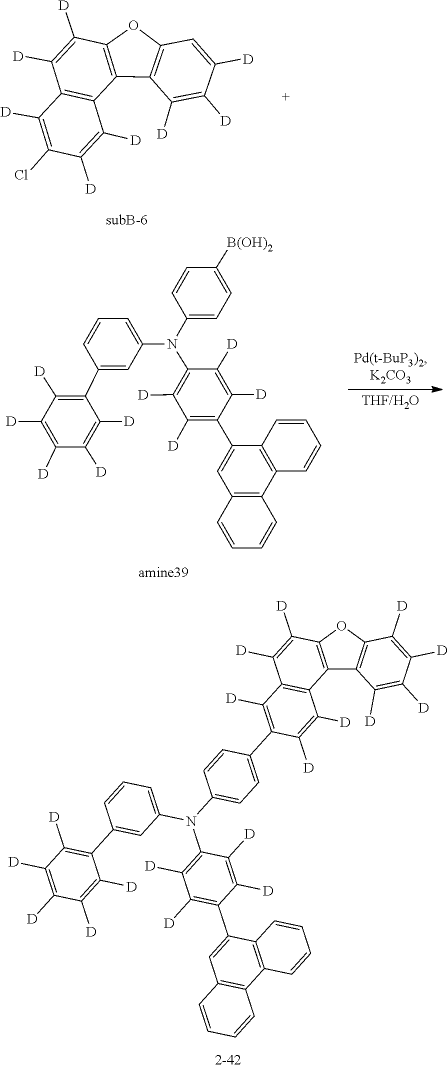 [SMILES: [2H]c1cc2oc3c([2H])c([2H])c4c([2H])c(Cl)c([2H])c([2H])c4c3c2c([2H])c1[2H] ; [2H]c1c([2H])c([2H])c(-c2cccc(N(c3ccc(B(O)O)cc3)c3c([2H])c([2H])c(-c4cc5ccccc5c5ccccc45)c([2H])c3[2H])c2)c([2H])c1[2H] ; [2H]c1c([2H])c([2H])c(-c2cccc(N(c3ccc(-c4c([2H])c([2H])c5c(c4[2H])c([2H])c([2H])c4oc6c([2H])c([2H])c([2H])c([2H])c6c45)cc3)c3c([2H])c([2H])c(-c4cc5ccccc5c5ccccc45)c([2H])c3[2H])c2)c([2H])c1[2H]]

Compound subB-6 (15 g, 57.5 mmol) and Compound amine39 (33.3 g, 60.4 mmol) were added to 300 ml of THF, and the mixture was stirred and refluxed. Then, potassium carbonate (23.9 g, 172.6 mmol) was dissolved in 72 ml of water and added thereto, and the mixture was sufficiently stirred and then bis(tri-tert-butylphosphine)palladium(0) (0.3 g, 0.6 mmol) was added. After reacting for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 30.3 g of Compound 2-42. (Yield: 72%, MS: [M+H]⁺=732)

### Synthesis Example 2-43

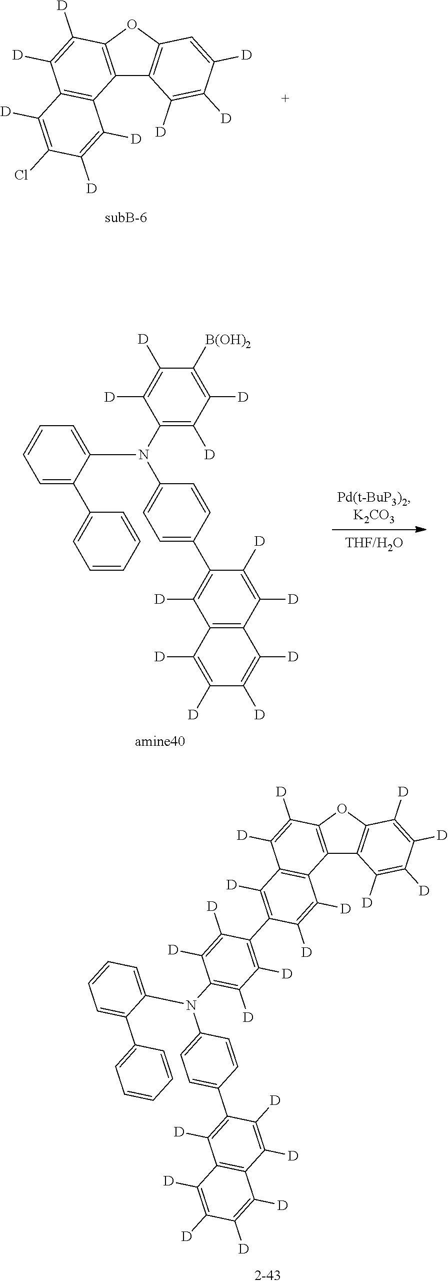 [SMILES: [2H]c1cc2oc3c([2H])c([2H])c4c([2H])c(Cl)c([2H])c([2H])c4c3c2c([2H])c1[2H] ; [2H]c1c([2H])c(N(c2ccc(-c3c([2H])c([2H])c4c([2H])c([2H])c([2H])c([2H])c4c3[2H])cc2)c2ccccc2-c2ccccc2)c([2H])c([2H])c1B(O)O ; [2H]c1c([2H])c(N(c2ccc(-c3c([2H])c([2H])c4c([2H])c([2H])c([2H])c([2H])c4c3[2H])cc2)c2ccccc2-c2ccccc2)c([2H])c([2H])c1-c1c([2H])c([2H])c2c(c1[2H])c([2H])c([2H])c1oc3c([2H])c([2H])c([2H])c([2H])c3c12]

Compound subB-6 (15 g, 57.5 mmol) and Compound amine40 (30.3 g, 60.4 mmol) were added to 300 ml of THF, and the mixture was stirred and refluxed. Then, potassium carbonate (23.9 g, 172.6 mmol) was dissolved in 72 ml of water and added thereto, and the mixture was sufficiently stirred and then bis(tri-tert-butylphosphine)palladium(0) (0.3 g, 0.6 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.7 g of Compound 2-43. (Yield: 68%, MS: [M+H]⁺=684)

### Synthesis Example 2-44

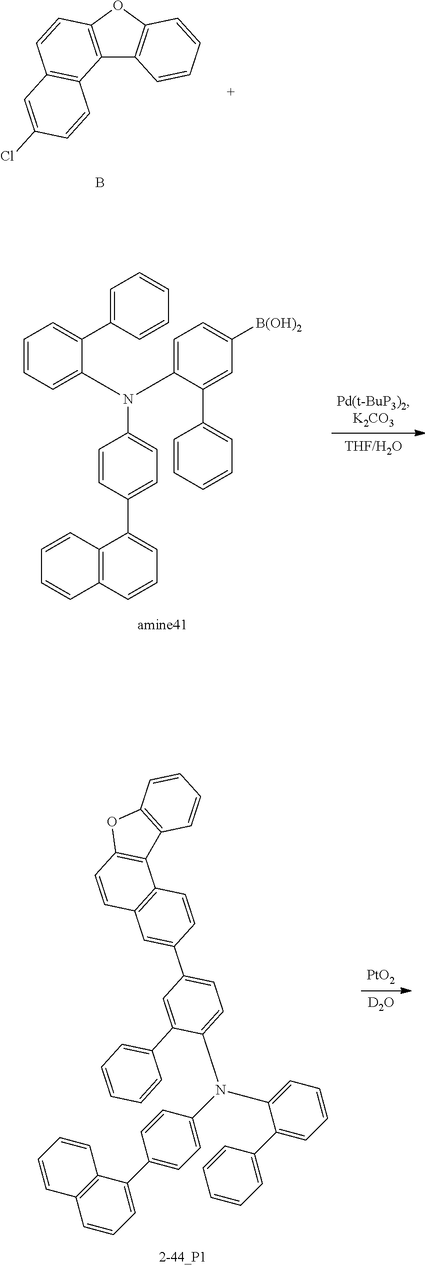

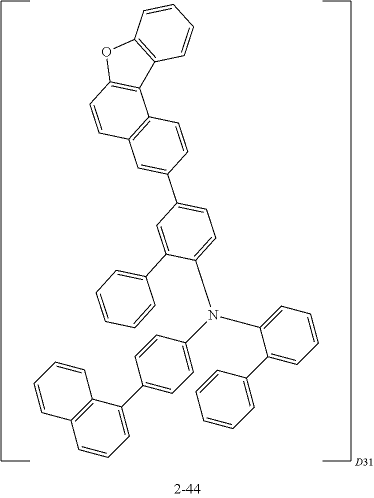

Compound B (15 g, 59.4 mmol) and Compound amine41 (35.4 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 32.5 g of Compound 2-44_P1. (Yield: 74%, MS: [M+H]⁺=740)

Compound 2-44_P1 (10 g, 13.5 mmol), PtO₂ (0.9 g, 4.1 mmol) and D₂O (68 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 4.6 g of Compound 2-44. (Yield: 44%, MS: [M+H]⁺=772)

### Synthesis Example 2-45

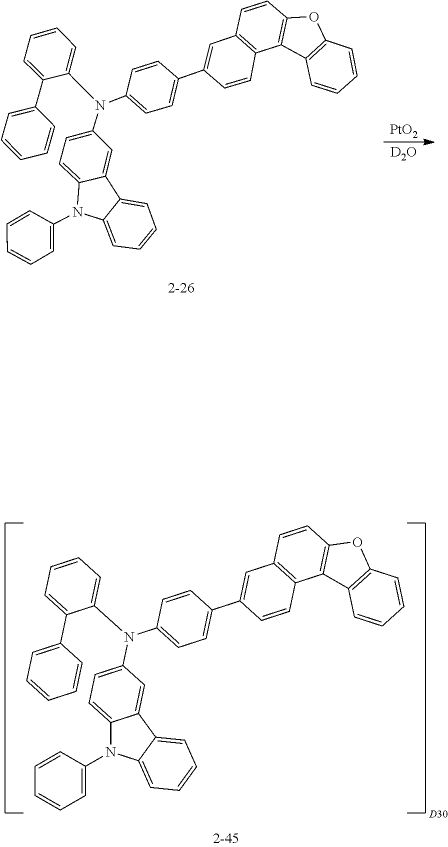

Compound 2-26 (10 g, 14.2 mmol), PtO₂ (1 g, 4.3 mmol) and D₂O (71 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 3.4 g of Compound 2-45. (Yield: 33%, MS: [M+H]⁺=734)

### Synthesis Example 2-46

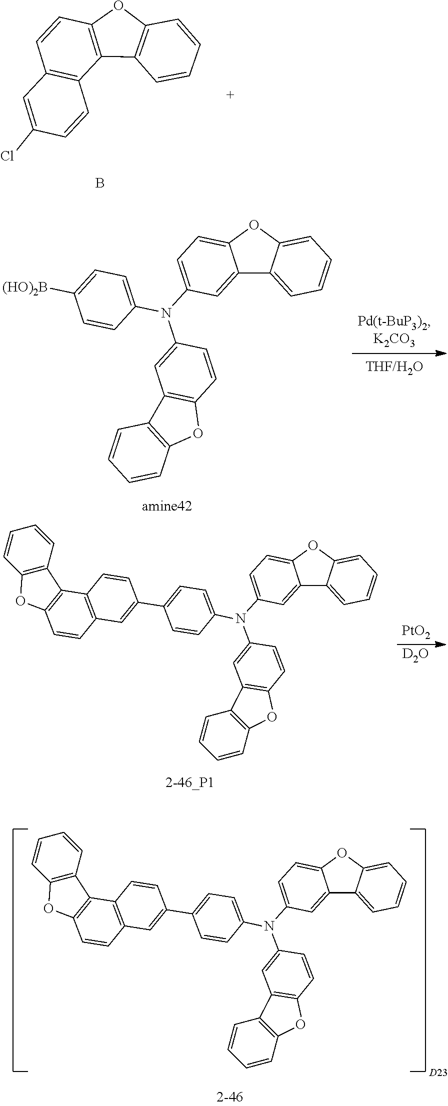

Compound B (15 g, 59.4 mmol) and Compound amine42 (29.3 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.8 g of Compound 2-46_P1. (Yield: 73%, MS: [M+H]⁺=642)

Compound 2-46_P1 (10 g, 15.6 mmol), PtO₂ (1.1 g, 4.7 mmol) and D₂O (78 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 3.2 g of Compound 2-46. (Yield: 31%, MS: [M+H]⁺=666)

### Synthesis Example 2-47

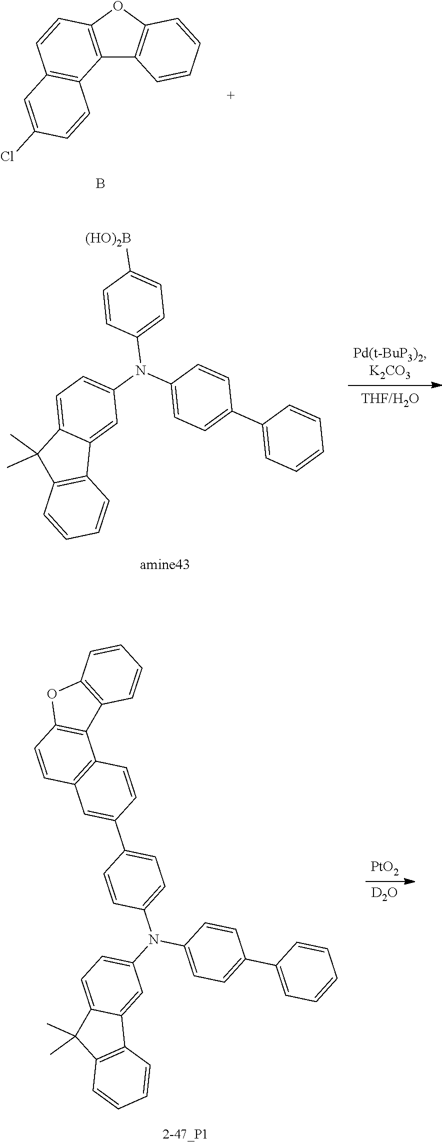

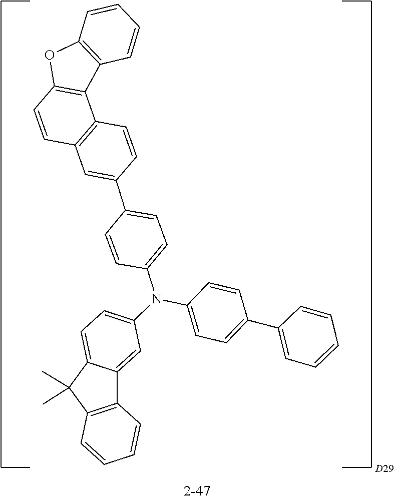

Compound B (15 g, 59.4 mmol) and Compound amine43 (30 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After reacting for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.4 g of Compound 2-47_P1. (Yield: 68%, MS: [M+H]⁺=654)

Compound 2-47_P1 (10 g, 15.3 mmol), PtO₂ (1 g, 4.6 mmol) and D₂O (76 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to give 4.6 g of Compound 2-47. (Yield: 44%, MS: [M+H]⁺=684)

### Example 1

A glass substrate on which a thin film of ITO (indium tin oxide) was coated in a thickness of 1000 Å was put into distilled water containing a detergent dissolved therein and ultrasonically washed. In this case, the detergent used was a product commercially available from Fischer Co. and the distilled water was one which had been twice filtered by using a filter commercially available from Millipore Co. The ITO was washed for 30 minutes, and ultrasonic washing was then repeated twice for 10 minutes by using distilled water. After the washing with distilled water was completed, the substrate was ultrasonically washed with isopropyl alcohol, acetone, and methanol solvent, and dried, after which it was transported to a plasma cleaner. Then, the substrate was cleaned with oxygen plasma for 5 minutes, and then transferred to a vacuum evaporator.

On the ITO transparent electrode thus prepared, the following compound HI-1 was formed in a thickness of 1150 Å as a hole injection layer, but the following compound A-1 was p-doped at a concentration of 1.5 wt. %. The following compound HT-1 was vacuum deposited on the hole injection layer to form a hole transport layer with a film thickness of 800 Å. Then, the following compound EB-1 was vacuum deposited on the hole transport layer to a film thickness of 150 Å to form an electron blocking layer. Then, the previously prepared Compound 1-1, Compound 2-2 and Compound Dp-7 were vacuum deposited in a weight ratio of 49:49:2 on the EB-1 deposited film to form a red light emitting layer with a film thickness of 400 Å. The following compound HB-1 was vacuum deposited on the light emitting layer to a film thickness of 30 Å to form a hole blocking layer. The following compound ET-1 and the following compound LiQ were vacuum deposited in a weight ratio of 2:1 on the hole blocking layer to form an electron injection and transport layer with a film thickness of 300 Å. Lithium fluoride (LiF) and aluminum were sequentially deposited to have a thickness of 12 Å and 1,000 Å, respectively, on the electron injection and transport layer, thereby forming a cathode.

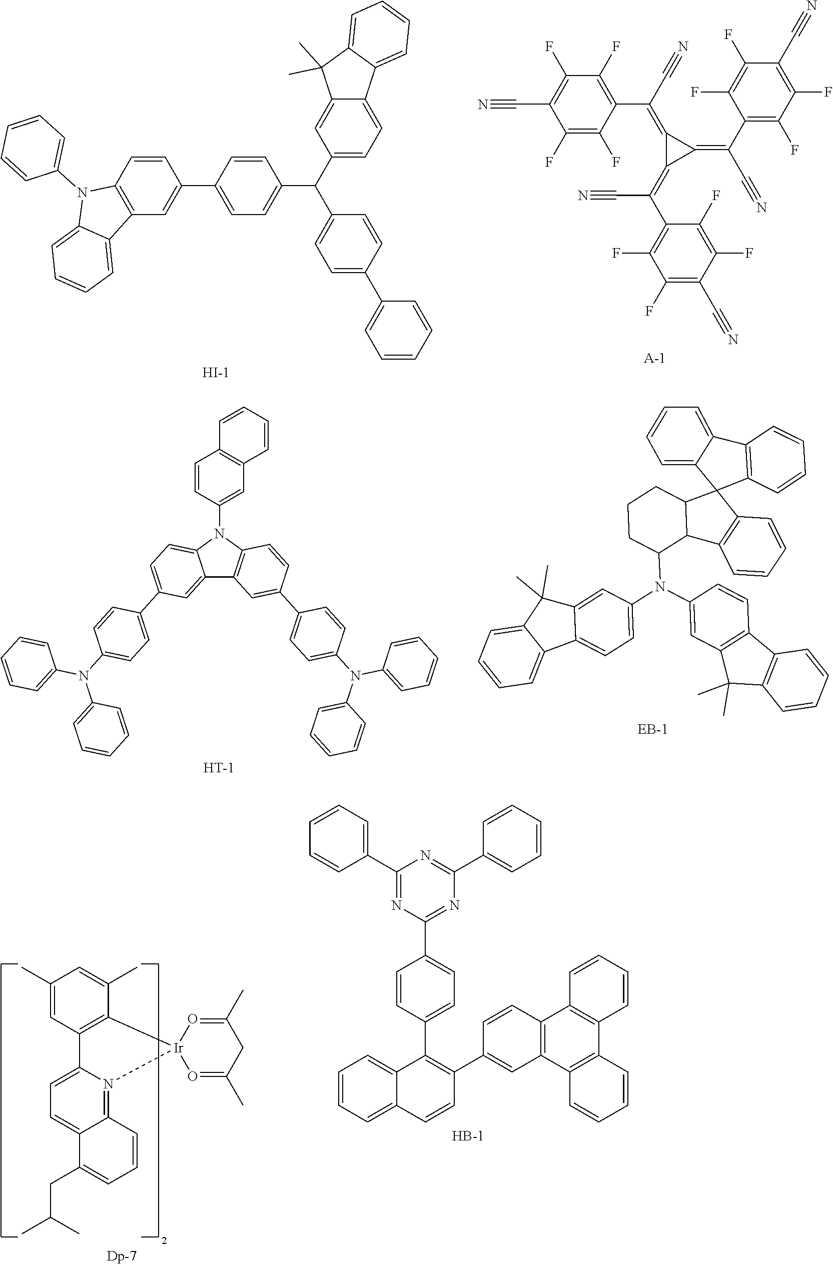

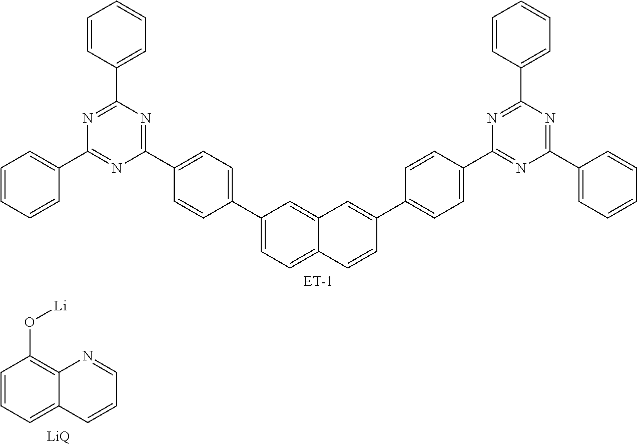

In the above-mentioned processes, the deposition rates of the organic materials were maintained at 0.4 to 0.7 Å/sec, the deposition rates of lithium fluoride and the aluminum of the cathode were maintained at 0.3 Å/sec and 2 Å/sec, respectively, and the degree of vacuum during the deposition was maintained at 2×10⁻⁷ to 5×10⁻⁶ torr, thereby manufacturing an organic light emitting device.

### Examples 2 to 190

The organic light emitting devices were manufactured in the same manner as in Example 1, except that in the organic light emitting device of Example 1, the compound of Chemical Formula 1 and the compound of Chemical Formula 2 shown in the following Tables 1 to 5 were co-deposited and used in a weight ratio of 1:1 instead of Compound 1-1 and Compound 2-2 as the first host and the second host.

### Comparative Examples 1 to 60

The organic light emitting devices were manufactured in the same manner as in Example 1, except that the following Comparative Compounds A-1 to A-12 1 was used instead of Compound 1-1 as the first host, and the compound of Chemical Formula 2 shown in Tables 6 and 7 below 1 was used instead of Compound 2-2 as the second host, which were co-deposited and used in a weight ratio of 1:1. Specific structures of Compounds A-1 to A-12 are as follows.

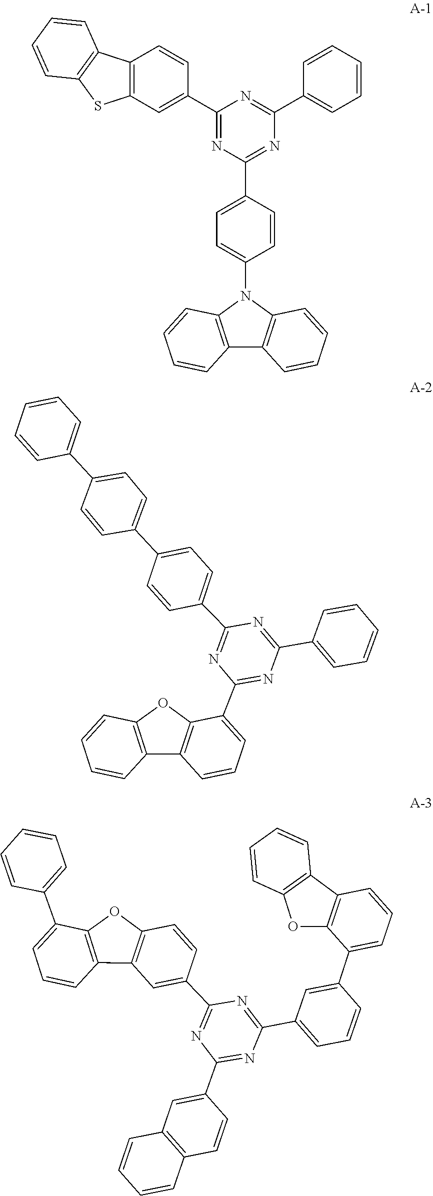 [SMILES: c1ccc(-c2nc(-c3ccc(-n4c5ccccc5c5ccccc54)cc3)nc(-c3ccc4c(c3)sc3ccccc34)n2)cc1 ; c1ccc(-c2ccc(-c3ccc(-c4nc(-c5ccccc5)nc(-c5cccc6c5oc5ccccc56)n4)cc3)cc2)cc1 ; c1ccc(-c2cccc3c2oc2ccc(-c4nc(-c5cccc(-c6cccc7c6oc6ccccc67)c5)nc(-c5ccc6ccccc6c5)n4)cc23)cc1]

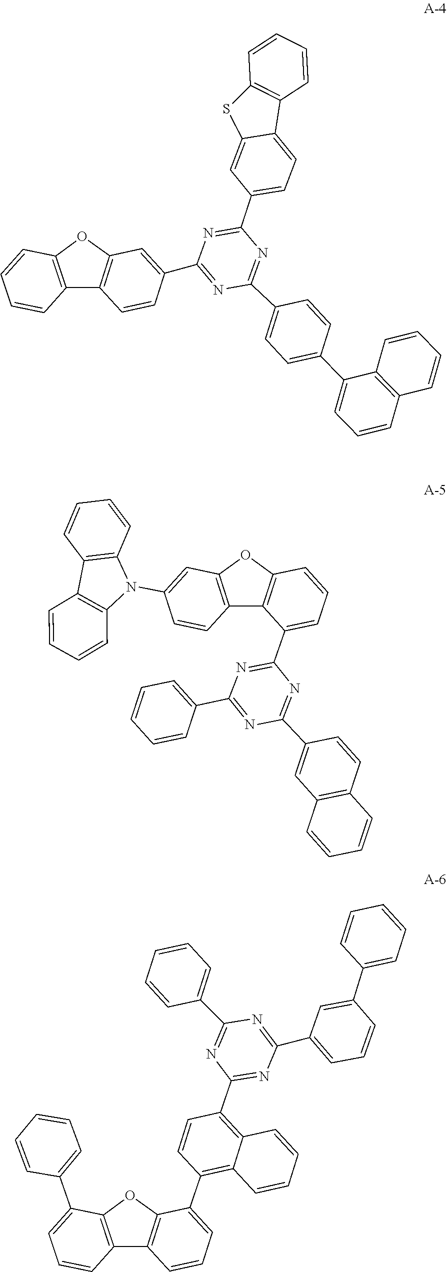 [SMILES: c1ccc2c(-c3ccc(-c4nc(-c5ccc6c(c5)oc5ccccc56)nc(-c5ccc6c(c5)sc5ccccc56)n4)cc3)cccc2c1 ; c1ccc(-c2nc(-c3ccc4ccccc4c3)nc(-c3cccc4oc5cc(-n6c7ccccc7c7ccccc76)ccc5c34)n2)cc1 ; c1ccc(-c2cccc(-c3nc(-c4ccccc4)nc(-c4ccc(-c5cccc6c5oc5c(-c7ccccc7)cccc56)c5ccccc45)n3)c2)cc1]

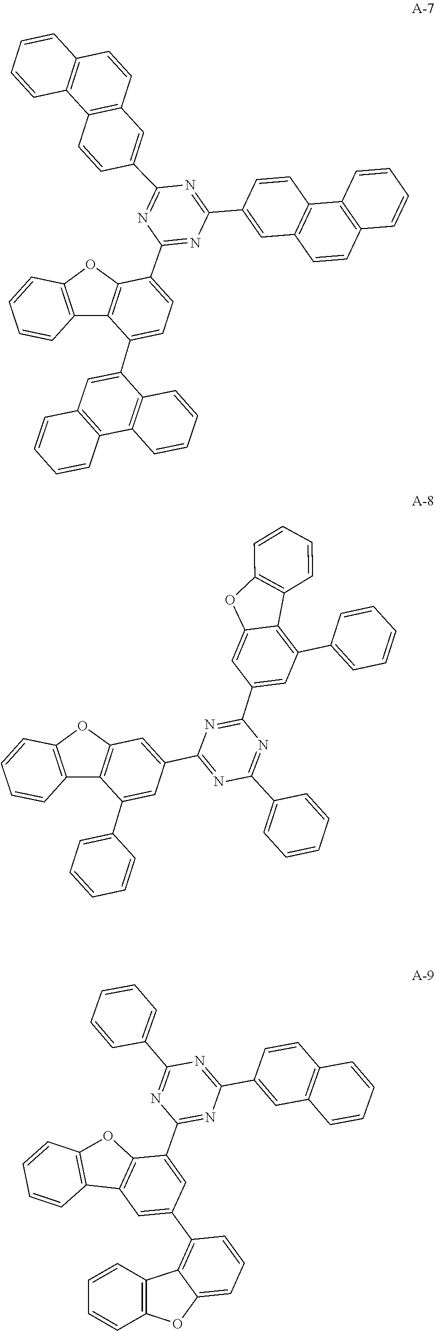 [SMILES: c1ccc2c(c1)ccc1cc(-c3nc(-c4ccc5c(ccc6ccccc65)c4)nc(-c4ccc(-c5cc6ccccc6c6ccccc56)c5c4oc4ccccc45)n3)ccc12 ; c1ccc(-c2nc(-c3cc(-c4ccccc4)c4c(c3)oc3ccccc34)nc(-c3cc(-c4ccccc4)c4c(c3)oc3ccccc34)n2)cc1 ; c1ccc(-c2nc(-c3ccc4ccccc4c3)nc(-c3cc(-c4cccc5oc6ccccc6c45)cc4c3oc3ccccc34)n2)cc1]

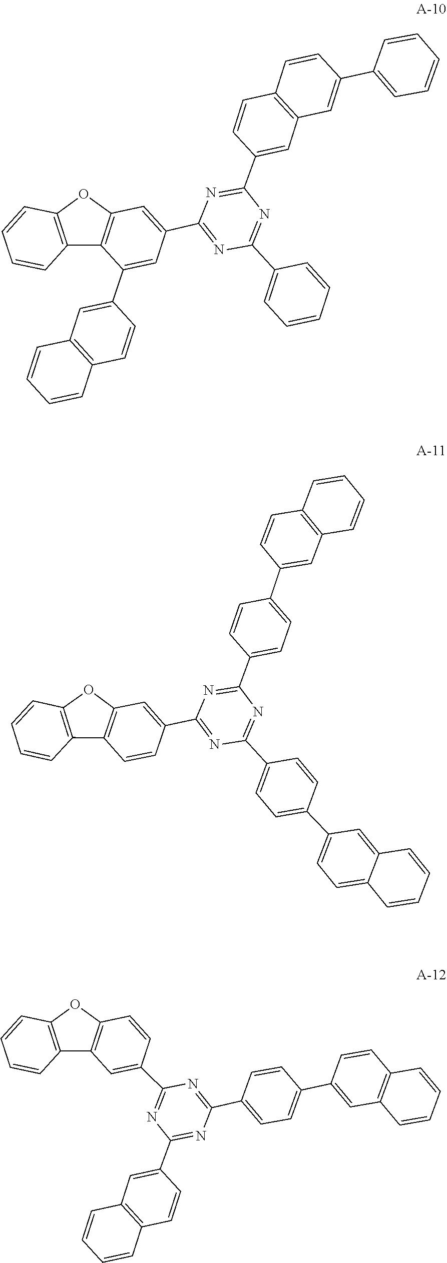 [SMILES: c1ccc(-c2ccc3ccc(-c4nc(-c5ccccc5)nc(-c5cc(-c6ccc7ccccc7c6)c6c(c5)oc5ccccc56)n4)cc3c2)cc1 ; c1ccc2cc(-c3ccc(-c4nc(-c5ccc(-c6ccc7ccccc7c6)cc5)nc(-c5ccc6c(c5)oc5ccccc56)n4)cc3)ccc2c1 ; c1ccc2cc(-c3ccc(-c4nc(-c5ccc6ccccc6c5)nc(-c5ccc6oc7ccccc7c6c5)n4)cc3)ccc2c1]

### Comparative Examples 61 to 172

The organic light emitting devices were manufactured in the same manner as in Example 1, except that the compound of Chemical Formula 1 shown in Tables 8 to 10 below was used instead of Compound 1-1 as the first host, and the following Comparative Compounds B-1 to B-14 were used instead of Compound 2-2 as the second host, which were co-deposited and used in a weight ratio of 1:1. Specific structures of Compounds B-1 to B-14 are as follows.

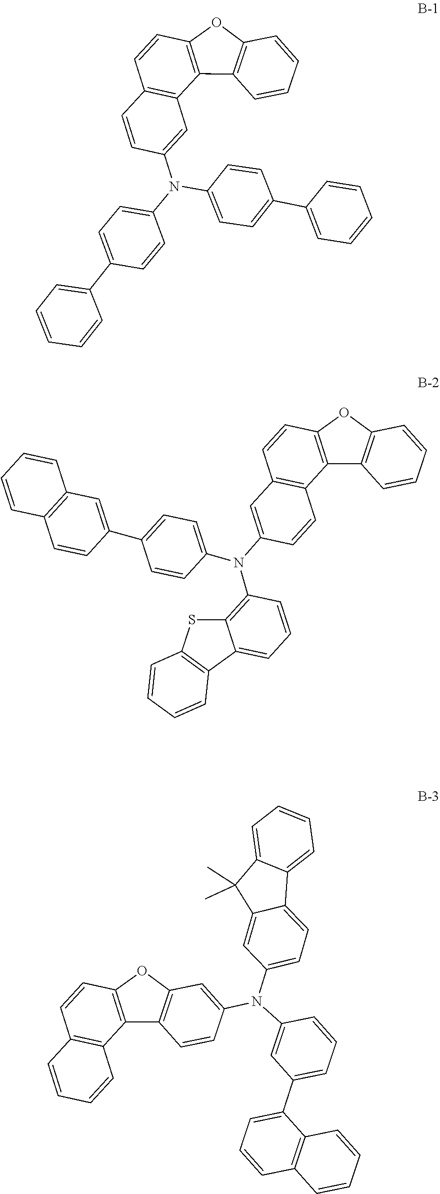 [SMILES: c1ccc(-c2ccc(N(c3ccc(-c4ccccc4)cc3)c3ccc4ccc5oc6ccccc6c5c4c3)cc2)cc1 ; c1ccc2cc(-c3ccc(N(c4ccc5c(ccc6oc7ccccc7c65)c4)c4cccc5c4sc4ccccc45)cc3)ccc2c1 ; CC1(C)c2ccccc2-c2ccc(N(c3cccc(-c4cccc5ccccc45)c3)c3ccc4c(c3)oc3ccc5ccccc5c34)cc21]

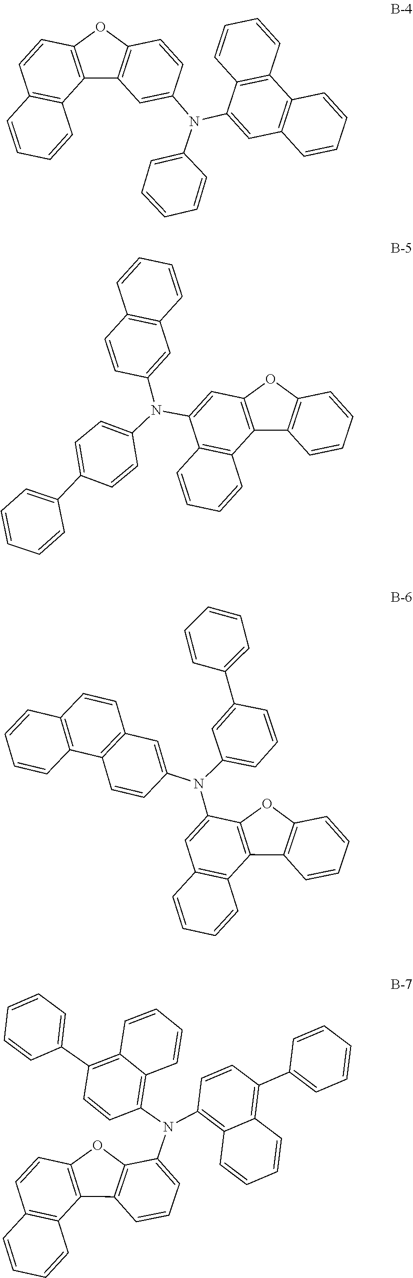 [SMILES: c1ccc(N(c2ccc3oc4ccc5ccccc5c4c3c2)c2cc3ccccc3c3ccccc23)cc1 ; c1ccc(-c2ccc(N(c3ccc4ccccc4c3)c3cc4oc5ccccc5c4c4ccccc34)cc2)cc1 ; c1ccc(-c2cccc(N(c3ccc4c(ccc5ccccc54)c3)c3cc4ccccc4c4c3oc3ccccc34)c2)cc1 ; c1ccc(-c2ccc(N(c3ccc(-c4ccccc4)c4ccccc34)c3cccc4c3oc3ccc5ccccc5c34)c3ccccc23)cc1]

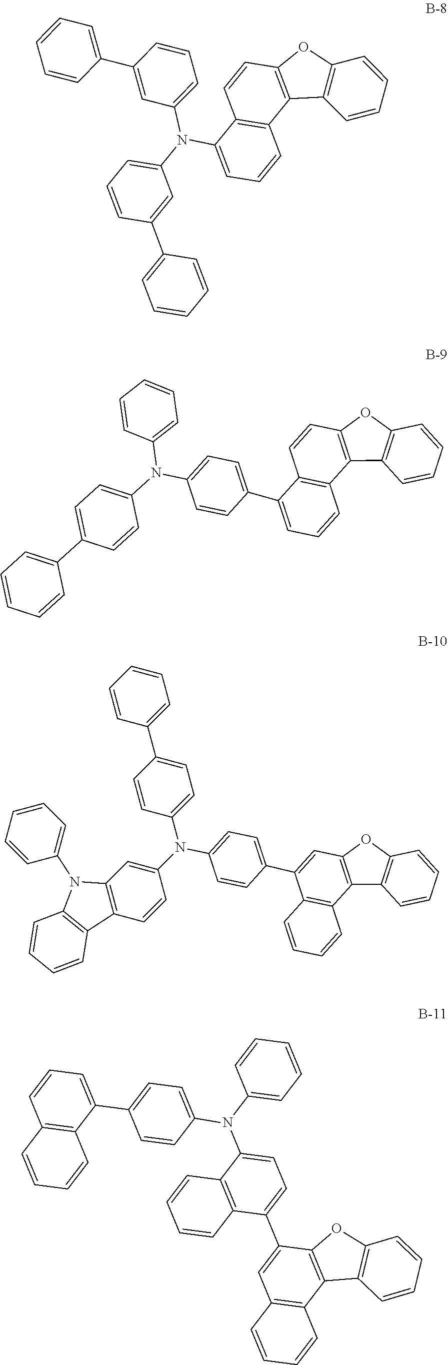 [SMILES: c1ccc(-c2cccc(N(c3cccc(-c4ccccc4)c3)c3cccc4c3ccc3oc5ccccc5c34)c2)cc1 ; c1ccc(-c2ccc(N(c3ccccc3)c3ccc(-c4cccc5c4ccc4oc6ccccc6c45)cc3)cc2)cc1 ; c1ccc(-c2ccc(N(c3ccc(-c4cc5oc6ccccc6c5c5ccccc45)cc3)c3ccc4c5ccccc5n(-c5ccccc5)c4c3)cc2)cc1 ; c1ccc(N(c2ccc(-c3cccc4ccccc34)cc2)c2ccc(-c3cc4ccccc4c4c3oc3ccccc34)c3ccccc23)cc1]

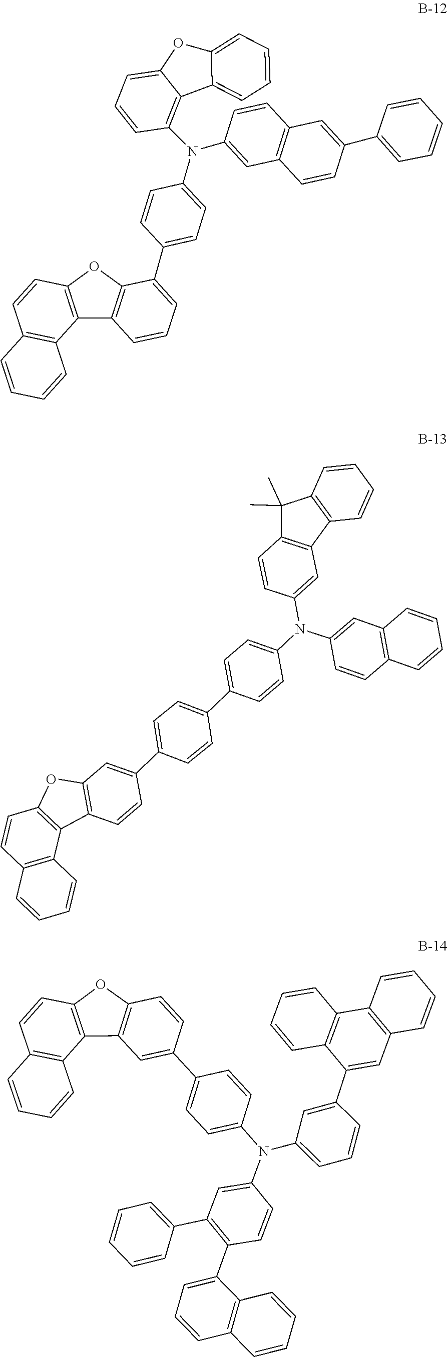 [SMILES: c1ccc(-c2ccc3cc(N(c4ccc(-c5cccc6c5oc5ccc7ccccc7c56)cc4)c4cccc5oc6ccccc6c45)ccc3c2)cc1 ; CC1(C)c2ccccc2-c2cc(N(c3ccc(-c4ccc(-c5ccc6c(c5)oc5ccc7ccccc7c56)cc4)cc3)c3ccc4ccccc4c3)ccc21 ; c1ccc(-c2cc(N(c3ccc(-c4ccc5oc6ccc7ccccc7c6c5c4)cc3)c3cccc(-c4cc5ccccc5c5ccccc45)c3)ccc2-c2cccc3ccccc23)cc1]

### Experimental Example

The voltage and efficiency were measured (15 mA/cm 2) by applying a current to the organic light emitting devices manufactured in Examples 1 to 190 and Comparative Examples 1 to 172, and the results are shown in Tables 1 to 10 below. Lifetime T95 was measured based on 7000 nits, and T95 means the time required for the lifetime to be reduced to 95% of the initial lifetime.

TABLE 1

| | | | | | Life- | Lumi- |
|---|---|---|---|---|---|---|
| | | | Driving | Effi- | time | nes- |
| | | Second | voltage | ciency | T95 | cent |
| Category | First host | host | (V) | (cd/A) | (hr) | color |
| Example | Compound | Compound | 3.62 | 22.57 | 281 | Red |
| 1 | 1-1 | 2-2 | | | | |
| Example | | Compound | 3.57 | 22.61 | 286 | Red |
| 2 | | 2-11 | | | | |
| Example | | Compound | 3.61 | 22.58 | 308 | Red |
| 3 | | 2-21 | | | | |
| Example | | Compound | 3.53 | 22.61 | 291 | Red |
| 4 | | 2-30 | | | | |
| Example | | Compound | 3.63 | 22.39 | 276 | Red |
| 5 | | 2-39 | | | | |
| Example | Compound | Compound | 3.56 | 21.21 | 216 | Red |
| 6 | 1-2 | 2-2 | | | | |
| Example | | Compound | 3.58 | 21.43 | 198 | Red |
| 7 | | 2-10 | | | | |
| Example | | Compound | 3.57 | 21.45 | 234 | Red |
| 8 | | 2-22 | | | | |
| Example | | Compound | 3.61 | 21.58 | 218 | Red |
| 9 | | 2-31 | | | | |
| Example | | Compound | 3.63 | 21.62 | 217 | Red |
| 10 | | 2-40 | | | | |
| Example | Compound | Compound | 3.53 | 21.03 | 215 | Red |
| 11 | 1-5 | 2-3 | | | | |
| Example | | Compound | 3.57 | 21.87 | 225 | Red |
| 12 | | 2-13 | | | | |
| Example | | Compound | 3.64 | 21.46 | 214 | Red |
| 13 | | 2-23 | | | | |
| Example | | Compound | 3.57 | 22.05 | 216 | Red |
| 14 | | 2-32 | | | | |
| Example | | Compound | 3.58 | 22.09 | 213 | Red |
| 15 | | 2-41 | | | | |
| Example | Compound | Compound | 3.77 | 20.62 | 216 | Red |
| 16 | 1-6 | 2-4 | | | | |
| Example | | Compound | 3.71 | 20.54 | 208 | Red |
| 17 | | 2-14 | | | | |
| Example | | Compound | 3.67 | 20.11 | 222 | Red |
| 18 | | 2-25 | | | | |
| Example | | Compound | 3.64 | 20.80 | 218 | Red |
| 19 | | 2-33 | | | | |
| Example | | Compound | 3.62 | 21.00 | 217 | Red |
| 20 | | 2-42 | | | | |
| Example | Compound | Compound | 3.72 | 20.41 | 215 | Red |
| 21 | 1-7 | 2-5 | | | | |
| Example | | Compound | 3.73 | 20.77 | 225 | Red |
| 22 | | 2-13 | | | | |
| Example | | Compound | 3.71 | 20.23 | 214 | Red |
| 23 | | 2-25 | | | | |
| Example | | Compound | 3.79 | 21.05 | 216 | Red |
| 24 | | 2-34 | | | | |
| Example | | Compound | 3.76 | 20.85 | 213 | Red |
| 25 | | 2-43 | | | | |
| Example | Compound | Compound | 3.42 | 23.69 | 322 | Red |
| 26 | 1-9 | 2-3 | | | | |
| Example | | Compound | 3.41 | 23.05 | 324 | Red |
| 27 | | 2-16 | | | | |
| Example | | Compound | 3.49 | 22.96 | 296 | Red |
| 28 | | 2-28 | | | | |
| Example | | Compound | 3.43 | 22.66 | 293 | Red |
| 29 | | 2-36 | | | | |
| Example | | Compound | 3.49 | 22.88 | 305 | Red |
| 30 | | 2-44 | | | | |
| Example | Compound | Compound | 3.51 | 23.10 | 303 | Red |
| 31 | 1-10 | 2-2 | | | | |
| Example | | Compound | 3.46 | 22.85 | 301 | Red |
| 32 | | 2-14 | | | | |
| Example | | Compound | 3.47 | 22.77 | 336 | Red |
| 33 | | 2-27 | | | | |
| Example | | Compound | 3.48 | 23.00 | 309 | Red |
| 34 | | 2-34 | | | | |
| Example | | Compound | 3.48 | 23.09 | 317 | Red |
| 35 | | 2-45 | | | | |
| Example | Compound | Compound | 3.51 | 22.81 | 290 | Red |
| 36 | 1-12 | 2-4 | | | | |
| Example | | Compound | 3.49 | 22.76 | 311 | Red |
| 37 | | 2-16 | | | | |
| Example | | Compound | 3.40 | 22.85 | 329 | Red |
| 38 | | 2-22 | | | | |
| Example | | Compound | 3.49 | 22.72 | 307 | Red |
| 39 | | 2-35 | | | | |
| Example | | Compound | 3.49 | 22.64 | 318 | Red |
| 40 | | 2-42 | | | | |
| Example | Compound | Compound | 3.68 | 20.38 | 227 | Red |
| 41 | 1-14 | 2-9 | | | | |
| Example | | Compound | 3.73 | 20.46 | 221 | Red |
| 42 | | 2-19 | | | | |
| Example | | Compound | 3.70 | 20.79 | 221 | Red |
| 43 | | 2-29 | | | | |
| Example | | Compound | 3.68 | 21.02 | 229 | Red |
| 44 | | 2-39 | | | | |
| Example | | Compound | 3.62 | 20.87 | 208 | Red |
| 45 | | 2-45 | | | | |

TABLE 2

| | | | | | Life- | Lumi- |
|---|---|---|---|---|---|---|
| | | | Driving | Effi- | time | nes- |
| | | Second | voltage | ciency | T95 | cent |
| Category | First host | host | (V) | (cd/A) | (hr) | color |
| Example | Compound | Compound | 3.62 | 20.40 | 225 | Red |
| 46 | 1-16 | 2-10 | | | | |
| Example | | Compound | 3.71 | 21.05 | 222 | Red |
| 47 | | 2-20 | | | | |
| Example | | Compound | 3.72 | 20.69 | 227 | Red |
| 48 | | 2-30 | | | | |
| Example | | Compound | 3.74 | 20.40 | 224 | Red |
| 49 | | 2-40 | | | | |
| Example | | Compound | 3.79 | 20.32 | 222 | Red |
| 50 | | 2-47 | | | | |
| Example | Compound | Compound | 3.53 | 22.37 | 275 | Red |
| 51 | 1-20 | 2-1 | | | | |
| Example | | Compound | 3.54 | 22.38 | 307 | Red |
| 52 | | 2-11 | | | | |
| Example | | Compound | 3.53 | 22.56 | 280 | Red |
| 53 | | 2-21 | | | | |
| Example | | Compound | 3.64 | 22.65 | 291 | Red |
| 54 | | 2-30 | | | | |
| Example | | Compound | 3.63 | 22.59 | 274 | Red |
| 55 | | 2-39 | | | | |
| Example | Compound | Compound | 3.58 | 22.20 | 272 | Red |
| 56 | 1-21 | 2-2 | | | | |
| Example | | Compound | 3.53 | 22.26 | 275 | Red |
| 57 | | 2-12 | | | | |
| Example | | Compound | 3.61 | 22.04 | 277 | Red |
| 58 | | 2-22 | | | | |
| Example | | Compound | 3.64 | 22.27 | 284 | Red |
| 59 | | 2-31 | | | | |
| Example | | Compound | 3.54 | 22.34 | 294 | Red |
| 60 | | 2-40 | | | | |
| Example | Compound | Compound | 3.60 | 22.05 | 208 | Red |
| 61 | 1-22 | 2-3 | | | | |
| Example | | Compound | 3.59 | 21.54 | 229 | Red |
| 62 | | 2-13 | | | | |
| Example | | Compound | 3.53 | 21.33 | 218 | Red |
| 63 | | 2-23 | | | | |
| Example | | Compound | 3.62 | 21.08 | 209 | Red |
| 64 | | 2-32 | | | | |
| Example | | Compound | 3.61 | 21.91 | 210 | Red |
| 65 | | 2-41 | | | | |
| Example | Compound | Compound | 3.61 | 21.23 | 217 | Red |
| 66 | 1-24 | 2-4 | | | | |
| Example | | Compound | 3.64 | 21.89 | 214 | Red |
| 67 | | 2-14 | | | | |
| Example | | Compound | 3.55 | 21.47 | 226 | Red |
| 68 | | 2-24 | | | | |
| Example | | Compound | 3.56 | 21.62 | 219 | Red |
| 69 | | 2-33 | | | | |
| Example | | Compound | 3.56 | 21.12 | 230 | Red |
| 70 | | 2-42 | | | | |
| Example | Compound | Compound | 3.77 | 20.85 | 225 | Red |
| 71 | 1-26 | 2-5 | | | | |
| Example | | Compound | 3.65 | 20.58 | 221 | Red |
| 72 | | 2-15 | | | | |
| Example | | Compound | 3.72 | 20.43 | 208 | Red |
| 73 | | 2-25 | | | | |
| Example | | Compound | 3.67 | 20.79 | 228 | Red |
| 74 | | 2-34 | | | | |
| Example | | Compound | 3.68 | 20.88 | 227 | Red |
| 75 | | 2-43 | | | | |
| Example | Compound | Compound | 3.68 | 20.10 | 222 | Red |
| 76 | 1-27 | 2-6 | | | | |
| Example | | Compound | 3.75 | 20.95 | 226 | Red |
| 77 | | 2-16 | | | | |
| Example | | Compound | 3.66 | 20.54 | 210 | Red |
| 78 | | 2-26 | | | | |
| Example | | Compound | 3.69 | 20.14 | 230 | Red |
| 79 | | 2-36 | | | | |
| Example | | Compound | 3.64 | 20.26 | 226 | Red |
| 80 | | 2-44 | | | | |
| Example | Compound | Compound | 3.54 | 21.20 | 236 | Red |
| 81 | 1-29 | 2-7 | | | | |
| Example | | Compound | 3.52 | 21.02 | 242 | Red |
| 82 | | 2-17 | | | | |
| Example | | Compound | 3.50 | 21.90 | 229 | Red |
| 83 | | 2-27 | | | | |
| Example | | Compound | 3.54 | 21.84 | 273 | Red |
| 84 | | 2-37 | | | | |
| Example | | Compound | 3.53 | 21.06 | 252 | Red |
| 85 | | 2-45 | | | | |
| Example | Compound | Compound | 3.50 | 23.00 | 318 | Red |
| 86 | 1-30 | 2-3 | | | | |
| Example | | Compound | 3.46 | 22.83 | 305 | Red |
| 87 | | 2-18 | | | | |
| Example | | Compound | 3.50 | 22.62 | 319 | Red |
| 88 | | 2-28 | | | | |
| Example | | Compound | 3.40 | 22.74 | 315 | Red |
| 89 | | 2-38 | | | | |
| Example | | Compound | 3.48 | 22.66 | 326 | Red |
| 90 | | 2-42 | | | | |

TABLE 3

| | | | | | Life- | Lumi- |
|---|---|---|---|---|---|---|
| | | | Driving | Effi- | time | nes- |
| | | Second | voltage | ciency | T95 | cent |
| Category | First host | host | (V) | (cd/A) | (hr) | color |
| Example | Compound | Compound | 3.44 | 22.97 | 318 | Red |
| 91 | 1-33 | 2-9 | | | | |
| Example | | Compound | 3.40 | 22.98 | 335 | Red |
| 92 | | 2-19 | | | | |
| Example | | Compound | 3.48 | 22.70 | 312 | Red |
| 93 | | 2-29 | | | | |
| Example | | Compound | 3.47 | 22.85 | 316 | Red |
| 94 | | 2-39 | | | | |
| Example | | Compound | 3.51 | 22.93 | 330 | Red |
| 95 | | 2-45 | | | | |
| Example | Compound | Compound | 3.60 | 22.13 | 273 | Red |
| 96 | 1-36 | 2-10 | | | | |
| Example | | Compound | 3.57 | 22.07 | 272 | Red |
| 97 | | 2-20 | | | | |
| Example | | Compound | 3.64 | 22.68 | 271 | Red |
| 98 | | 2-30 | | | | |
| Example | | Compound | 3.62 | 22.54 | 294 | Red |
| 99 | | 2-40 | | | | |
| Example | | Compound | 3.64 | 22.07 | 287 | Red |
| 100 | | 2-47 | | | | |
| Example | Compound | Compound | 3.62 | 22.72 | 253 | Red |
| 101 | 1-37 | 2-1 | | | | |
| Example | | Compound | 3.61 | 22.70 | 297 | Red |
| 102 | | 2-11 | | | | |
| Example | | Compound | 3.57 | 22.68 | 276 | Red |
| 103 | | 2-21 | | | | |
| Example | | Compound | 3.58 | 22.61 | 288 | Red |
| 104 | | 2-30 | | | | |
| Example | | Compound | 3.59 | 22.13 | 292 | Red |
| 105 | | 2-39 | | | | |
| Example | Compound | Compound | 3.45 | 22.62 | 331 | Red |
| 106 | 1-39 | 2-2 | | | | |
| Example | | Compound | 3.49 | 22.70 | 300 | Red |
| 107 | | 2-14 | | | | |
| Example | | Compound | 3.51 | 22.77 | 312 | Red |
| 108 | | 2-22 | | | | |
| Example | | Compound | 3.44 | 22.75 | 301 | Red |
| 109 | | 2-31 | | | | |
| Example | | Compound | 3.43 | 22.82 | 337 | Red |
| 110 | | 2-40 | | | | |
| Example | Compound | Compound | 3.57 | 21.88 | 209 | Red |
| 111 | 1-42 | 2-3 | | | | |
| Example | | Compound | 3.60 | 21.31 | 228 | Red |
| 112 | | 2-13 | | | | |
| Example | | Compound | 3.55 | 21.61 | 222 | Red |
| 113 | | 2-23 | | | | |
| Example | | Compound | 3.56 | 22.09 | 208 | Red |
| 114 | | 2-32 | | | | |
| Example | | Compound | 3.61 | 21.97 | 208 | Red |
| 115 | | 2-41 | | | | |
| Example | Compound | Compound | 3.63 | 22.08 | 212 | Red |
| 116 | 1-43 | 2-4 | | | | |
| Example | | Compound | 3.62 | 21.39 | 214 | Red |
| 117 | | 2-14 | | | | |
| Example | | Compound | 3.57 | 21.99 | 210 | Red |
| 118 | | 2-24 | | | | |
| Example | | Compound | 3.57 | 21.95 | 217 | Red |
| 119 | | 2-33 | | | | |
| Example | | Compound | 3.61 | 21.94 | 224 | Red |
| 120 | | 2-42 | | | | |
| Example | Compound | Compound | 3.61 | 20.59 | 297 | Red |
| 121 | 1-44 | 2-5 | | | | |
| Example | | Compound | 3.45 | 20.43 | 295 | Red |
| 122 | | 2-15 | | | | |
| Example | | Compound | 3.62 | 20.18 | 285 | Red |
| 123 | | 2-25 | | | | |
| Example | | Compound | 3.48 | 20.64 | 286 | Red |
| 124 | | 2-34 | | | | |
| Example | | Compound | 3.55 | 20.38 | 275 | Red |
| 125 | | 2-43 | | | | |
| Example | Compound | Compound | 3.53 | 21.88 | 235 | Red |
| 126 | 1-45 | 2-6 | | | | |
| Example | | Compound | 3.55 | 21.31 | 237 | Red |
| 127 | | 2-16 | | | | |
| Example | | Compound | 3.53 | 21.61 | 235 | Red |
| 128 | | 2-26 | | | | |
| Example | | Compound | 3.56 | 22.09 | 272 | Red |
| 129 | | 2-36 | | | | |
| Example | | Compound | 3.56 | 21.97 | 244 | Red |
| 130 | | 2-44 | | | | |
| Example | Compound | Compound | 3.45 | 22.77 | 310 | Red |
| 131 | 1-46 | 2-2 | | | | |
| Example | | Compound | 3.44 | 23.32 | 355 | Red |
| 132 | | 2-14 | | | | |
| Example | | Compound | 3.50 | 23.22 | 337 | Red |
| 133 | | 2-27 | | | | |
| Example | | Compound | 3.42 | 23.12 | 343 | Red |
| 134 | | 2-37 | | | | |
| Example | | Compound | 3.40 | 23.11 | 313 | Red |
| 135 | | 2-45 | | | | |

TABLE 4

| | | | | | Life- | Lumi- |
|---|---|---|---|---|---|---|
| | | | Driving | Effi- | time | nes- |
| | | Second | voltage | ciency | T95 | cent |
| Category | First host | host | (V) | (cd/A) | (hr) | color |
| Example | Compound | Compound | 3.49 | 22.82 | 323 | Red |
| 136 | 1-47 | 2-8 | | | | |
| Example | | Compound | 3.41 | 23.14 | 311 | Red |
| 137 | | 2-18 | | | | |
| Example | | Compound | 3.42 | 22.94 | 299 | Red |
| 138 | | 2-28 | | | | |
| Example | | Compound | 3.47 | 22.95 | 306 | Red |
| 139 | | 2-38 | | | | |
| Example | | Compound | 3.48 | 22.66 | 352 | Red |
| 140 | | 2-42 | | | | |
| Example | Compound | Compound | 3.50 | 22.04 | 231 | Red |
| 141 | 1-49 | 2-9 | | | | |
| Example | | Compound | 3.50 | 21.18 | 262 | Red |
| 142 | | 2-19 | | | | |
| Example | | Compound | 3.49 | 21.44 | 240 | Red |
| 143 | | 2-29 | | | | |
| Example | | Compound | 3.49 | 21.83 | 267 | Red |
| 144 | | 2-39 | | | | |
| Example | | Compound | 3.51 | 22.05 | 243 | Red |
| 145 | | 2-45 | | | | |
| Example | Compound | Compound | 3.53 | 21.34 | 230 | Red |
| 146 | 1-50 | 2-10 | | | | |
| Example | | Compound | 3.50 | 21.55 | 252 | Red |
| 147 | | 2-20 | | | | |
| Example | | Compound | 3.51 | 21.21 | 253 | Red |
| 148 | | 2-30 | | | | |
| Example | | Compound | 3.52 | 21.83 | 242 | Red |
| 149 | | 2-40 | | | | |
| Example | | Compound | 3.52 | 21.34 | 246 | Red |
| 150 | | 2-47 | | | | |
| Example | Compound | Compound | 3.58 | 22.04 | 212 | Red |
| 151 | 1-51 | 2-1 | | | | |
| Example | | Compound | 3.57 | 22.06 | 253 | Red |
| 152 | | 2-11 | | | | |
| Example | | Compound | 3.63 | 21.44 | 228 | Red |
| 153 | | 2-21 | | | | |
| Example | | Compound | 3.64 | 21.83 | 230 | Red |
| 154 | | 2-30 | | | | |
| Example | | Compound | 3.55 | 22.05 | 219 | Red |
| 155 | | 2-39 | | | | |
| Example | Compound | Compound | 3.53 | 21.34 | 217 | Red |
| 156 | 1-52 | 2-2 | | | | |
| Example | | Compound | 3.64 | 21.55 | 224 | Red |
| 157 | | 2-12 | | | | |
| Example | | Compound | 3.59 | 21.21 | 217 | Red |
| 158 | | 2-22 | | | | |
| Example | | Compound | 3.56 | 21.83 | 215 | Red |
| 159 | | 2-31 | | | | |
| Example | | Compound | 3.57 | 21.34 | 214 | Red |
| 160 | | 2-40 | | | | |
| Example | Compound | Compound | 3.41 | 23.67 | 351 | Red |
| 161 | 1-53 | 2-3 | | | | |
| Example | | Compound | 3.44 | 22.93 | 291 | Red |
| 162 | | 2-13 | | | | |
| Example | | Compound | 3.49 | 23.43 | 314 | Red |
| 163 | | 2-23 | | | | |
| Example | | Compound | 3.47 | 23.60 | 305 | Red |
| 164 | | 2-32 | | | | |
| Example | | Compound | 3.45 | 22.96 | 312 | Red |
| 165 | | 2-41 | | | | |
| Example | Compound | Compound | 3.49 | 22.98 | 336 | Red |
| 166 | 1-55 | 2-4 | | | | |
| Example | | Compound | 3.42 | 23.35 | 365 | Red |
| 167 | | 2-14 | | | | |
| Example | | Compound | 3.51 | 22.80 | 295 | Red |
| 168 | | 2-22 | | | | |
| Example | | Compound | 3.50 | 22.83 | 300 | Red |
| 169 | | 2-33 | | | | |
| Example | | Compound | 3.41 | 22.96 | 314 | Red |
| 170 | | 2-42 | | | | |
| Example | Compound | Compound | 3.47 | 22.69 | 306 | Red |
| 171 | 1-57 | 2-4 | | | | |
| Example | | Compound | 3.47 | 22.79 | 303 | Red |
| 172 | | 2-15 | | | | |
| Example | | Compound | 3.43 | 22.73 | 329 | Red |
| 173 | | 2-25 | | | | |
| Example | | Compound | 3.45 | 22.64 | 296 | Red |
| 174 | | 2-34 | | | | |
| Example | | Compound | 3.47 | 22.74 | 301 | Red |
| 175 | | 2-43 | | | | |
| Example | Compound | Compound | 3.55 | 21.90 | 290 | Red |
| 176 | 1-58 | 2-6 | | | | |
| Example | | Compound | 3.59 | 22.14 | 287 | Red |
| 177 | | 2-16 | | | | |
| Example | | Compound | 3.64 | 22.08 | 276 | Red |
| 178 | | 2-26 | | | | |
| Example | | Compound | 3.60 | 22.07 | 292 | Red |
| 179 | | 2-36 | | | | |
| Example | | Compound | 3.62 | 22.34 | 289 | Red |
| 180 | | 2-44 | | | | |

TABLE 5

| | | | | | Life- | Lumi- |
|---|---|---|---|---|---|---|
| | | | Driving | Effi- | time | nes- |
| | | Second | voltage | ciency | T95 | cent |
| Category | First host | host | (V) | (cd/A) | (hr) | color |
| Example | Compound | Compound | 3.50 | 23.07 | 331 | Red |
| 181 | 1-59 | 2-7 | | | | |
| Example | | Compound | 3.44 | 22.76 | 296 | Red |
| 182 | | 2-17 | | | | |
| Example | | Compound | 3.40 | 22.98 | 308 | Red |
| 183 | | 2-23 | | | | |
| Example | | Compound | 3.42 | 22.80 | 306 | Red |
| 184 | | 2-37 | | | | |
| Example | | Compound | 3.46 | 22.86 | 304 | Red |
| 185 | | 2-45 | | | | |
| Example | Compound | Compound | 3.53 | 21.61 | 215 | Red |
| 186 | 1-61 | 2-8 | | | | |
| Example | | Compound | 3.54 | 21.84 | 217 | Red |
| 187 | | 2-18 | | | | |
| Example | | Compound | 3.59 | 21.80 | 230 | Red |
| 188 | | 2-28 | | | | |
| Example | | Compound | 3.55 | 21.35 | 219 | Red |
| 189 | | 2-38 | | | | |
| Example | | Compound | 3.55 | 21.31 | 211 | Red |
| 190 | | 2-42 | | | | |

TABLE 6

| | | | | | Life- | Lumi- |
|---|---|---|---|---|---|---|
| | | | Driving | Effi- | time | nes- |
| | | Second | voltage | ciency | T95 | cent |
| Category | First host | host | (V) | (cd/A) | (hr) | color |
| Comparative | Compound | Compound | 3.96 | 18.36 | 146 | Red |
| Example 1 | A-1 | 2-2 | | | | |
| Comparative | | Compound | 3.95 | 18.15 | 174 | Red |
| Example 2 | | 2-11 | | | | |
| Comparative | | Compound | 3.95 | 18.37 | 181 | Red |
| Example 3 | | 2-21 | | | | |
| Comparative | | Compound | 3.92 | 17.83 | 148 | Red |
| Example 4 | | 2-30 | | | | |
| Comparative | | Compound | 3.88 | 18.18 | 176 | Red |
| Example 5 | | 2-39 | | | | |
| Comparative | Compound | Compound | 3.89 | 17.39 | 154 | Red |
| Example 6 | A-2 | 2-2 | | | | |
| Comparative | | Compound | 3.93 | 17.73 | 162 | Red |
| Example 7 | | 2-10 | | | | |
| Comparative | | Compound | 3.94 | 17.74 | 198 | Red |
| Example 8 | | 2-22 | | | | |
| Comparative | | Compound | 3.92 | 17.66 | 177 | Red |
| Example 9 | | 2-31 | | | | |
| Comparative | | Compound | 3.93 | 17.93 | 179 | Red |
| Example 10 | | 2-40 | | | | |
| Comparative | Compound | Compound | 4.11 | 16.20 | 98 | Red |
| Example 11 | A-3 | 2-3 | | | | |
| Comparative | | Compound | 4.14 | 15.92 | 128 | Red |
| Example 12 | | 2-13 | | | | |
| Comparative | | Compound | 4.23 | 14.82 | 141 | Red |
| Example 13 | | 2-23 | | | | |
| Comparative | | Compound | 4.19 | 14.62 | 175 | Red |
| Example 14 | | 2-32 | | | | |
| Comparative | | Compound | 4.14 | 15.44 | 169 | Red |
| Example 15 | | 2-41 | | | | |
| Comparative | Compound | Compound | 4.14 | 14.57 | 133 | Red |
| Example 16 | A-4 | 2-4 | | | | |
| Comparative | | Compound | 4.07 | 15.79 | 179 | Red |
| Example 17 | | 2-14 | | | | |
| Comparative | | Compound | 4.07 | 16.34 | 130 | Red |
| Example 18 | | 2-25 | | | | |
| Comparative | | Compound | 4.11 | 15.45 | 187 | Red |
| Example 19 | | 2-33 | | | | |
| Comparative | | Compound | 4.15 | 15.86 | 174 | Red |
| Example 20 | | 2-42 | | | | |
| Comparative | Compound | Compound | 3.93 | 17.93 | 159 | Red |
| Example 21 | A-5 | 2-5 | | | | |
| Comparative | | Compound | 3.93 | 18.02 | 173 | Red |
| Example 22 | | 2-13 | | | | |
| Comparative | | Compound | 3.89 | 17.40 | 158 | Red |
| Example 23 | | 2-25 | | | | |
| Comparative | | Compound | 3.92 | 17.87 | 171 | Red |
| Example 24 | | 2-34 | | | | |
| Comparative | | Compound | 3.94 | 17.54 | 185 | Red |
| Example 25 | | 2-43 | | | | |
| Comparative | Compound | Compound | 3.92 | 17.80 | 134 | Red |
| Example 26 | A-6 | 2-3 | | | | |
| Comparative | | Compound | 3.95 | 17.84 | 182 | Red |
| Example 27 | | 2-16 | | | | |
| Comparative | | Compound | 3.90 | 18.03 | 143 | Red |
| Example 28 | | 2-28 | | | | |
| Comparative | | Compound | 3.90 | 18.16 | 171 | Red |
| Example 29 | | 2-36 | | | | |
| Comparative | | Compound | 3.89 | 17.86 | 189 | Red |
| Example 30 | | 2-44 | | | | |

TABLE 7

| | | | | | Life- | Lumi- |
|---|---|---|---|---|---|---|
| | | | Driving | Effi- | time | nes- |
| | | Second | voltage | ciency | T95 | cent |
| Category | First host | host | (V) | (cd/A) | (hr) | color |
| Comparative | Compound | Compound | 3.91 | 17.41 | 139 | Red |
| Example 31 | A-7 | 2-2 | | | | |
| Comparative | | Compound | 3.88 | 17.46 | 179 | Red |
| Example 32 | | 2-14 | | | | |
| Comparative | | Compound | 3.94 | 17.88 | 140 | Red |
| Example 33 | | 2-27 | | | | |
| Comparative | | Compound | 3.89 | 17.89 | 186 | Red |
| Example 34 | | 2-34 | | | | |
| Comparative | | Compound | 3.95 | 17.44 | 172 | Red |
| Example 35 | | 2-45 | | | | |
| Comparative | Compound | Compound | 3.91 | 17.20 | 141 | Red |
| Example 36 | A-8 | 2-4 | | | | |
| Comparative | | Compound | 3.89 | 16.54 | 179 | Red |
| Example 37 | | 2-16 | | | | |
| Comparative | | Compound | 3.93 | 16.63 | 186 | Red |
| Example 38 | | 2-22 | | | | |
| Comparative | | Compound | 3.88 | 17.43 | 172 | Red |
| Example 39 | | 2-35 | | | | |
| Comparative | | Compound | 3.89 | 17.45 | 187 | Red |
| Example 40 | | 2-42 | | | | |
| Comparative | Compound | Compound | 3.88 | 18.08 | 151 | Red |
| Example 41 | A-9 | 2-9 | | | | |
| Comparative | | Compound | 3.88 | 18.10 | 175 | Red |
| Example 42 | | 2-19 | | | | |
| Comparative | | Compound | 3.93 | 17.35 | 144 | Red |
| Example 43 | | 2-29 | | | | |
| Comparative | | Compound | 3.92 | 17.37 | 172 | Red |
| Example 44 | | 2-39 | | | | |
| Comparative | | Compound | 3.89 | 17.56 | 188 | Red |
| Example 45 | | 2-45 | | | | |
| Comparative | Compound | Compound | 3.91 | 17.49 | 179 | Red |
| Example 46 | A-10 | 2-10 | | | | |
| Comparative | | Compound | 3.94 | 17.51 | 173 | Red |
| Example 47 | | 2-20 | | | | |
| Comparative | | Compound | 3.92 | 17.75 | 147 | Red |
| Example 48 | | 2-30 | | | | |
| Comparative | | Compound | 3.91 | 17.61 | 182 | Red |
| Example 49 | | 2-40 | | | | |
| Comparative | | Compound | 3.89 | 18.01 | 177 | Red |
| Example 50 | | 2-47 | | | | |
| Comparative | Compound | Compound | 3.95 | 16.55 | 127 | Red |
| Example 51 | A-11 | 2-1 | | | | |
| Comparative | | Compound | 3.97 | 16.53 | 169 | Red |
| Example 52 | | 2-11 | | | | |
| Comparative | | Compound | 3.92 | 16.47 | 174 | Red |
| Example 53 | | 2-21 | | | | |
| Comparative | | Compound | 3.88 | 16.84 | 121 | Red |
| Example 54 | | 2-30 | | | | |
| Comparative | | Compound | 3.94 | 16.52 | 180 | Red |
| Example 55 | | 2-39 | | | | |
| Comparative | Compound | Compound | 4.15 | 16.06 | 86 | Red |
| Example 56 | A-12 | 2-2 | | | | |
| Comparative | | Compound | 4.19 | 14.82 | 147 | Red |
| Example 57 | | 2-12 | | | | |
| Comparative | | Compound | 4.21 | 14.56 | 125 | Red |
| Example 58 | | 2-22 | | | | |
| Comparative | | Compound | 4.12 | 16.26 | 146 | Red |
| Example 59 | | 2-31 | | | | |
| Comparative | | Compound | 4.22 | 14.94 | 155 | Red |
| Example 60 | | 2-40 | | | | |

TABLE 8

| | | | | | Life- | Lumi- |
|---|---|---|---|---|---|---|
| | | | Driving | Effi- | time | nes- |
| | | Second | voltage | ciency | T95 | cent |
| Category | First host | host | (V) | (cd/A) | (hr) | color |
| Comparative | Compound | Compound | 3.92 | 16.50 | 138 | Red |
| Example 61 | 1-11 | B-1 | | | | |
| Comparative | Compound | | 3.94 | 16.57 | 121 | Red |
| Example 62 | 1-14 | | | | | |
| Comparative | Compound | | 3.95 | 16.80 | 112 | Red |
| Example 63 | 1-29 | | | | | |
| Comparative | Compound | | 3.88 | 17.44 | 127 | Red |
| Example 64 | 1-44 | | | | | |
| Comparative | Compound | | 3.90 | 17.45 | 152 | Red |
| Example 65 | 1-53 | | | | | |
| Comparative | Compound | | 3.93 | 16.37 | 122 | Red |
| Example 66 | 1-2 | | | | | |
| Comparative | Compound | | 3.88 | 16.40 | 118 | Red |
| Example 67 | 1-16 | | | | | |
| Comparative | Compound | | 3.93 | 17.26 | 142 | Red |
| Example 68 | 1-30 | | | | | |
| Comparative | Compound | Compound | 4.12 | 16.10 | 124 | Red |
| Example 69 | 1-45 | B-2 | | | | |
| Comparative | Compound | | 4.04 | 15.86 | 194 | Red |
| Example 70 | 1-55 | | | | | |
| Comparative | Compound | | 4.08 | 16.20 | 113 | Red |
| Example 71 | 1-5 | | | | | |
| Comparative | Compound | | 4.06 | 16.35 | 128 | Red |
| Example 72 | 1-20 | | | | | |
| Comparative | Compound | | 4.08 | 16.06 | 121 | Red |
| Example 73 | 1-33 | | | | | |
| Comparative | Compound | | 4.06 | 15.85 | 164 | Red |
| Example 74 | 1-46 | | | | | |
| Comparative | Compound | | 4.13 | 16.28 | 155 | Red |
| Example 75 | 1-57 | | | | | |
| Comparative | Compound | | 4.17 | 16.10 | 123 | Red |
| Example 76 | 1-6 | | | | | |
| Comparative | Compound | Compound | 3.92 | 16.50 | 138 | Red |
| Example 77 | 1-36 | B-3 | | | | |
| Comparative | Compound | | 3.94 | 16.57 | 161 | Red |
| Example 78 | 1-47 | | | | | |
| Comparative | Compound | | 3.95 | 16.80 | 142 | Red |
| Example 79 | 1-58 | | | | | |
| Comparative | Compound | | 3.88 | 17.44 | 137 | Red |
| Example 80 | 1-7 | | | | | |
| Comparative | Compound | | 3.90 | 17.45 | 142 | Red |
| Example 81 | 1-22 | | | | | |
| Comparative | Compound | | 3.93 | 16.37 | 132 | Red |
| Example 82 | 1-37 | | | | | |
| Comparative | Compound | | 3.88 | 16.40 | 138 | Red |
| Example 83 | 1-49 | | | | | |
| Comparative | Compound | | 3.93 | 17.26 | 172 | Red |
| Example 84 | 1-59 | | | | | |
| Comparative | Compound | Compound | 4.16 | 16.10 | 108 | Red |
| Example 85 | 1-4 | B-4 | | | | |
| Comparative | Compound | | 4.15 | 15.30 | 88 | Red |
| Example 86 | 1-42 | | | | | |
| Comparative | Compound | | 4.02 | 17.39 | 104 | Red |
| Example 87 | 1-9 | | | | | |
| Comparative | Compound | | 4.16 | 16.35 | 83 | Red |
| Example 88 | 1-31 | | | | | |
| Comparative | Compound | | 4.11 | 16.06 | 85 | Red |
| Example 89 | 1-13 | | | | | |
| Comparative | Compound | | 4.17 | 16.08 | 121 | Red |
| Example 90 | 1-43 | | | | | |
| Comparative | Compound | | 4.15 | 16.28 | 90 | Red |
| Example 91 | 1-26 | | | | | |
| Comparative | Compound | | 4.03 | 17.40 | 13 | Red |
| Example 92 | 1-53 | | | | | |
| Comparative | Compound | Compound | 3.96 | 17.37 | 184 | Red |
| Example 93 | 1-9 | B-5 | | | | |
| Comparative | Compound | | 3.90 | 16.98 | 187 | Red |
| Example 94 | 1-24 | | | | | |
| Comparative | Compound | | 3.89 | 16.97 | 136 | Red |
| Example 95 | 1-39 | | | | | |
| Comparative | Compound | | 3.94 | 17.41 | 135 | Red |
| Example 96 | 1-50 | | | | | |
| Comparative | Compound | | 3.89 | 17.41 | 171 | Red |
| Example 97 | 1-61 | | | | | |
| Comparative | Compound | | 3.88 | 16.41 | 172 | Red |
| Example 98 | 1-10 | | | | | |
| Comparative | Compound | | 3.89 | 16.55 | 134 | Red |
| Example 99 | 1-26 | | | | | |
| Comparative | Compound | | 3.91 | 16.48 | 144 | Red |
| Example | 1-42 | | | | | |
| 100 | | | | | | |

TABLE 9

| | | | | | Life- | Lumi- |
|---|---|---|---|---|---|---|
| | | | Driving | Effi- | time | nes- |
| | | Second | voltage | ciency | T95 | cent |
| Category | First host | host | (V) | (cd/A) | (hr) | color |
| Comparative | Compound | Compound | 3.92 | 17.95 | 163 | Red |
| Example | 1-51 | B-6 | | | | |
| 101 | | | | | | |
| Comparative | Compound | | 3.91 | 18.11 | 183 | Red |
| Example | 1-12 | | | | | |
| 102 | | | | | | |
| Comparative | Compound | | 3.88 | 17.35 | 147 | Red |
| Example | 1-27 | | | | | |
| 103 | | | | | | |
| Comparative | Compound | | 3.90 | 17.55 | 178 | Red |
| Example | 1-43 | | | | | |
| 104 | | | | | | |
| Comparative | Compound | | 3.93 | 18.04 | 161 | Red |
| Example | 1-52 | | | | | |
| 105 | | | | | | |
| Comparative | Compound | | 3.94 | 17.46 | 181 | Red |
| Example | 1-1 | | | | | |
| 106 | | | | | | |
| Comparative | Compound | | 3.89 | 17.73 | 151 | Red |
| Example | 1-14 | | | | | |
| 107 | | | | | | |
| Comparative | Compound | | 3.88 | 17.30 | 162 | Red |
| Example | 1-29 | | | | | |
| 108 | | | | | | |
| Comparative | Compound | Compound | 3.96 | 17.37 | 164 | Red |
| Example | 1-1 | B-7 | | | | |
| 109 | | | | | | |
| Comparative | Compound | | 3.90 | 16.98 | 147 | Red |
| Example | 1-14 | | | | | |
| 110 | | | | | | |
| Comparative | Compound | | 3.89 | 16.97 | 136 | Red |
| Example | 1-29 | | | | | |
| 111 | | | | | | |
| Comparative | Compound | | 3.94 | 17.41 | 135 | Red |
| Example | 1-44 | | | | | |
| 112 | | | | | | |
| Comparative | Compound | | 3.89 | 17.41 | 161 | Red |
| Example | 1-53 | | | | | |
| 113 | | | | | | |
| Comparative | Compound | | 3.88 | 16.41 | 132 | Red |
| Example | 1-2 | | | | | |
| 114 | | | | | | |
| Comparative | Compound | | 3.89 | 16.55 | 134 | Red |
| Example | 1-16 | | | | | |
| 115 | | | | | | |
| Comparative | Compound | | 3.91 | 16.48 | 164 | Red |
| Example | 1-30 | | | | | |
| 116 | | | | | | |
| Comparative | Compound | Compound | 3.92 | 17.95 | 163 | Red |
| Example | 1-45 | B-8 | | | | |
| 117 | | | | | | |
| Comparative | Compound | | 3.91 | 18.11 | 183 | Red |
| Example | 1-55 | | | | | |
| 118 | | | | | | |
| Comparative | Compound | | 3.88 | 17.35 | 147 | Red |
| Example | 1-5 | | | | | |
| 119 | | | | | | |
| Comparative | Compound | | 3.90 | 17.55 | 178 | Red |
| Example | 1-20 | | | | | |
| 120 | | | | | | |
| Comparative | Compound | | 3.93 | 18.04 | 161 | Red |
| Example | 1-33 | | | | | |
| 121 | | | | | | |
| Comparative | Compound | | 3.94 | 17.46 | 186 | Red |
| Example | 1-46 | | | | | |
| 122 | | | | | | |
| Comparative | Compound | | 3.89 | 17.73 | 178 | Red |
| Example | 1-57 | | | | | |
| 123 | | | | | | |
| Comparative | Compound | | 3.88 | 17.30 | 162 | Red |
| Example | 1-6 | | | | | |
| 124 | | | | | | |
| Comparative | Compound | Compound | 4.05 | 15.03 | 113 | Red |
| Example | 1-36 | B-9 | | | | |
| 125 | | | | | | |
| Comparative | Compound | | 4.09 | 16.02 | 187 | Red |
| Example | 1-47 | | | | | |
| 126 | | | | | | |
| Comparative | Compound | | 4.09 | 16.00 | 129 | Red |
| Example | 1-58 | | | | | |
| 127 | | | | | | |
| Comparative | Compound | | 4.08 | 15.18 | 117 | Red |
| Example | 1-7 | | | | | |
| 128 | | | | | | |
| Comparative | Compound | | 4.16 | 15.36 | 132 | Red |
| Example | 1-22 | | | | | |
| 129 | | | | | | |
| Comparative | Compound | | 4.13 | 15.61 | 135 | Red |
| Example | 1-37 | | | | | |
| 130 | | | | | | |
| Comparative | Compound | | 4.11 | 14.96 | 123 | Red |
| Example | 1-49 | | | | | |
| 131 | | | | | | |
| Comparative | Compound | | 4.17 | 14.69 | 184 | Red |
| Example | 1-59 | | | | | |
| 132 | | | | | | |
| Comparative | Compound | Compound | 3.92 | 18.95 | 153 | Red |
| Example | 1-4 | B-10 | | | | |
| 133 | | | | | | |
| Comparative | Compound | | 3.91 | 18.11 | 153 | Red |
| Example | 1-42 | | | | | |
| 134 | | | | | | |
| Comparative | Compound | | 3.88 | 19.35 | 167 | Red |
| Example | 1-9 | | | | | |
| 135 | | | | | | |
| Comparative | Compound | | 3.90 | 17.55 | 148 | Red |
| Example | 1-31 | | | | | |
| 136 | | | | | | |
| Comparative | Compound | | 3.93 | 18.04 | 151 | Red |
| Example | 1-13 | | | | | |
| 137 | | | | | | |
| Comparative | Compound | | 3.91 | 18.97 | 183 | Red |
| Example | 1-43 | | | | | |
| 138 | | | | | | |
| Comparative | Compound | | 3.89 | 17.73 | 150 | Red |
| Example | 1-26 | | | | | |
| 139 | | | | | | |
| Comparative | Compound | | 3.86 | 19.37 | 194 | Red |
| Example | 1-53 | | | | | |
| 140 | | | | | | |

TABLE 10

| | | | | | Life- | Lumi- |
|---|---|---|---|---|---|---|
| | | | Driving | Effi- | time | nes- |
| | | Second | voltage | ciency | T95 | cent |
| Category | First host | host | (V) | (cd/A) | (hr) | color |
| Comparative | Compound | Compound | 3.96 | 17.37 | 184 | Red |
| Example | 1-9 | B-11 | | | | |
| 141 | | | | | | |
| Comparative | Compound | | 3.90 | 16.98 | 147 | Red |
| Example | 1-24 | | | | | |
| 142 | | | | | | |
| Comparative | Compound | | 3.89 | 16.97 | 176 | Red |
| Example | 1-39 | | | | | |
| 143 | | | | | | |
| Comparative | Compound | | 3.94 | 17.41 | 135 | Red |
| Example | 1-50 | | | | | |
| 144 | | | | | | |
| Comparative | Compound | | 3.89 | 17.41 | 141 | Red |
| Example | 1-61 | | | | | |
| 145 | | | | | | |
| Comparative | Compound | | 3.88 | 16.41 | 182 | Red |
| Example | 1-10 | | | | | |
| 146 | | | | | | |
| Comparative | Compound | | 3.89 | 16.55 | 137 | Red |
| Example | 1-26 | | | | | |
| 147 | | | | | | |
| Comparative | Compound | | 3.91 | 16.48 | 144 | Red |
| Example | 1-42 | | | | | |
| 148 | | | | | | |
| Comparative | Compound | Compound | 3.92 | 17.95 | 193 | Red |
| Example | 1-1 | B-12 | | | | |
| 149 | | | | | | |
| Comparative | Compound | | 3.91 | 18.11 | 153 | Red |
| Example | 1-14 | | | | | |
| 150 | | | | | | |
| Comparative | Compound | | 3.88 | 17.35 | 147 | Red |
| Example | 1-29 | | | | | |
| 151 | | | | | | |
| Comparative | Compound | | 3.90 | 17.55 | 138 | Red |
| Example | 1-44 | | | | | |
| 152 | | | | | | |
| Comparative | Compound | | 3.93 | 18.04 | 179 | Red |
| Example | 1-53 | | | | | |
| 153 | | | | | | |
| Comparative | Compound | | 3.94 | 17.46 | 171 | Red |
| Example | 1-2 | | | | | |
| 154 | | | | | | |
| Comparative | Compound | | 3.89 | 17.73 | 151 | Red |
| Example | 1-16 | | | | | |
| 155 | | | | | | |
| Comparative | Compound | | 3.88 | 17.30 | 186 | Red |
| Example | 1-30 | | | | | |
| 156 | | | | | | |
| Comparative | Compound | Compound | 3.97 | 17.20 | 144 | Red |
| Example | 1-45 | B-13 | | | | |
| 157 | | | | | | |
| Comparative | Compound | | 3.89 | 17.50 | 188 | Red |
| Example | 1-55 | | | | | |
| 158 | | | | | | |
| Comparative | Compound | | 3.90 | 16.55 | 137 | Red |
| Example | 1-5 | | | | | |
| 159 | | | | | | |
| Comparative | Compound | | 3.88 | 16.90 | 147 | Red |
| Example | 1-20 | | | | | |
| 160 | | | | | | |
| Comparative | Compound | | 3.95 | 17.27 | 143 | Red |
| Example | 1-33 | | | | | |
| 161 | | | | | | |
| Comparative | Compound | | 3.95 | 17.43 | 182 | Red |
| Example | 1-46 | | | | | |
| 162 | | | | | | |
| Comparative | Compound | | 3.91 | 16.44 | 182 | Red |
| Example | 1-57 | | | | | |
| 163 | | | | | | |
| Comparative | Compound | | 3.90 | 16.53 | 148 | Red |
| Example | 1-6 | | | | | |
| 164 | | | | | | |
| Comparative | Compound | Compound | 4.13 | 15.31 | 134 | Red |
| Example | 1-36 | B-14 | | | | |
| 165 | | | | | | |
| Comparative | Compound | | 4.13 | 15.40 | 176 | Red |
| Example | 1-47 | | | | | |
| 166 | | | | | | |
| Comparative | Compound | | 4.16 | 15.10 | 132 | Red |
| Example | 1-58 | | | | | |
| 167 | | | | | | |
| Comparative | Compound | | 4.10 | 14.96 | 120 | Red |
| Example | 1-7 | | | | | |
| 168 | | | | | | |
| Comparative | Compound | | 4.09 | 16.13 | 122 | Red |
| Example | 1-22 | | | | | |
| 169 | | | | | | |
| Comparative | Compound | | 4.05 | 16.07 | 112 | Red |
| Example | 1-37 | | | | | |
| 170 | | | | | | |
| Comparative | Compound | | 4.15 | 16.39 | 128 | Red |
| Example | 1-49 | | | | | |
| 171 | | | | | | |
| Comparative | Compound | | 4.17 | 14.91 | 176 | Red |
| Example | 1-59 | | | | | |
| 172 | | | | | | |

When a current was applied to the organic light emitting devices manufactured in Examples 1 to 190 and Comparative Examples 1 to 172, the results shown in Table 1 Table 10 were obtained.

When Comparative Example Compounds A-1 to A-12 and the compound of Chemical Formula 2 of the present disclosure were co-deposited together and used as a red light emitting layer as shown in Table 6 and Table 7, the result showed that generally, the driving voltage increased and the efficiency and lifetime decreased as compared with one embodiment of the present disclosure. Even when Comparative Example Compounds B-1 to B-20 and the compound of Chemical Formula 1 of the present disclosure were co-deposited together and used as a red light emitting layer as shown in Table 8 to Table 10, the result showed that the driving voltage increased and the efficiency and lifetime decreased. From the above results, it can be inferred that the reason why

the driving voltage is improved and the efficiency and lifetime are increased is that when the compound of Chemical Formula 1 which is the first host of the present disclosure, and the Compound of Chemical Formula 2 which is the second host of the present disclosure, were used in combination, energy transfer to the red dopant in the red light emitting layer is made more favorable.

Therefore, it can be confirmed that since the combination of the compound of Chemical Formula 1 and the compound of Chemical Formula 2 of the present disclosure achieves a more stable balance in the light emitting layer than the combination with the comparative compound, electrons and holes combine to form excitons, which greatly increases efficiency and lifetime. From this, it was confirmed that when the compound of Chemical Formula 1 and the compound of Chemical Formula 2 of the present disclosure were co-deposited and used as a host for the red light emitting layer, the driving voltage, luminous efficiency, and lifetime characteristics of the organic light emitting device could be improved.

<Description of Symbols>

| | |
|---|---|
| 1: substrate | 2: anode |
| 3: light emitting layer | 4: cathode |
| 5: hole injection layer | 6: hole transport layer |
| 7: electron blocking layer | 8: hole blocking layer |
| 9: electron injection and transport layer | |

1. An organic light emitting device, comprising
an anode;
a cathode; and
a light emitting layer interposed between the anode and the cathode,
wherein the light emitting layer includes a compound of the following Chemical Formula 1 and a compound of the following Chemical Formula 2: 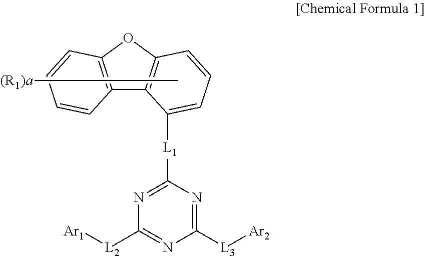
wherein in the Chemical Formula 1:
Ar₁ and Ar₂ are each independently a substituted or unsubstituted C₆₋₆₀ aryl or a substituted or unsubstituted C₂₋₆₀ heteroaryl containing at least one heteroatom selected from the group consisting of N, O and S;
L₁ to L₃ are each independently a single bond or a substituted or unsubstituted C₆₋₆₀ arylene;
R₁ is each independently hydrogen or deuterium; and
a is an integer of 0 to 7; 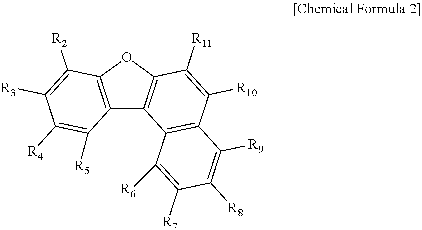
wherein in the Chemical Formula 2:
R₂ to R₆ and R₉ to R₁₁ are each independently hydrogen or deuterium;
any one of R₇ and R₈ is 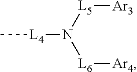
and the other is hydrogen or deuterium;
Ar₃ and Ar₄ are each independently a substituted or unsubstituted C₆₋₆₀ aryl or a substituted or unsubstituted C₂₋₆₀ heteroaryl containing at least one heteroatom selected from the group consisting of N, O and S;
L₄ is a substituted or unsubstituted phenylene, a substituted or unsubstituted biphenyldiyl, or a substituted or unsubstituted naphthalenediyl; and
L₅ and L₆ are each independently a single bond, a substituted or unsubstituted C₆₋₆₀ arylene, or a substituted or unsubstituted C₂₋₆₀ heteroarylene containing at least one heteroatom selected from the group consisting of N, O and S.

2. The organic light emitting device according to claim 1, wherein:
the compound of Chemical Formula 1 comprises at least one deuterium substituent.

3. The organic light emitting device according to claim 1, wherein:
Ar₁ and Ar₂ are each independently phenyl, triphenylsilyl phenyl, biphenylyl, terphenylyl, naphthyl, phenanthrenyl, dibenzofuranyl, or dibenzothiophenyl; and
one or more of the hydrogens of Ar₁ and Ar₂ each independently can be replaced with deuterium.

4. The organic light emitting device according to claim 1, wherein
L₁ to L₃ are each independently a single bond, phenylene, biphenyldiyl, or naphthalenediyl; and
one or more of the hydrogens of L₁ to L₃ are each independently replaced with deuterium.

5. The organic light emitting device according to claim 1, wherein:
the compound of Chemical Formula 1 is any one compound selected from the group consisting of:

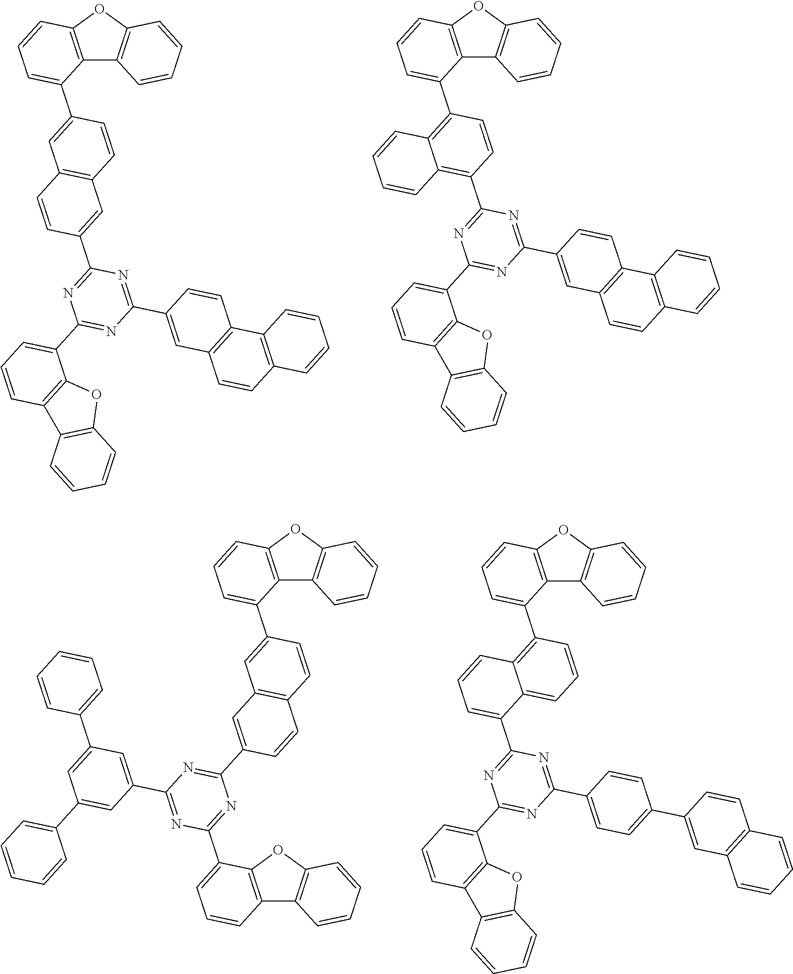 [SMILES: c1ccc2c(c1)ccc1cc(-c3nc(-c4ccc5cc(-c6cccc7oc8ccccc8c67)ccc5c4)nc(-c4cccc5c4oc4ccccc45)n3)ccc12 ; c1ccc2c(c1)ccc1cc(-c3nc(-c4ccc(-c5cccc6oc7ccccc7c56)c5ccccc45)nc(-c4cccc5c4oc4ccccc45)n3)ccc12 ; c1ccc(-c2cc(-c3ccccc3)cc(-c3nc(-c4ccc5ccc(-c6cccc7oc8ccccc8c67)cc5c4)nc(-c4cccc5c4oc4ccccc45)n3)c2)cc1 ; c1ccc2cc(-c3ccc(-c4nc(-c5cccc6c(-c7cccc8oc9ccccc9c78)cccc56)nc(-c5cccc6c5oc5ccccc56)n4)cc3)ccc2c1]

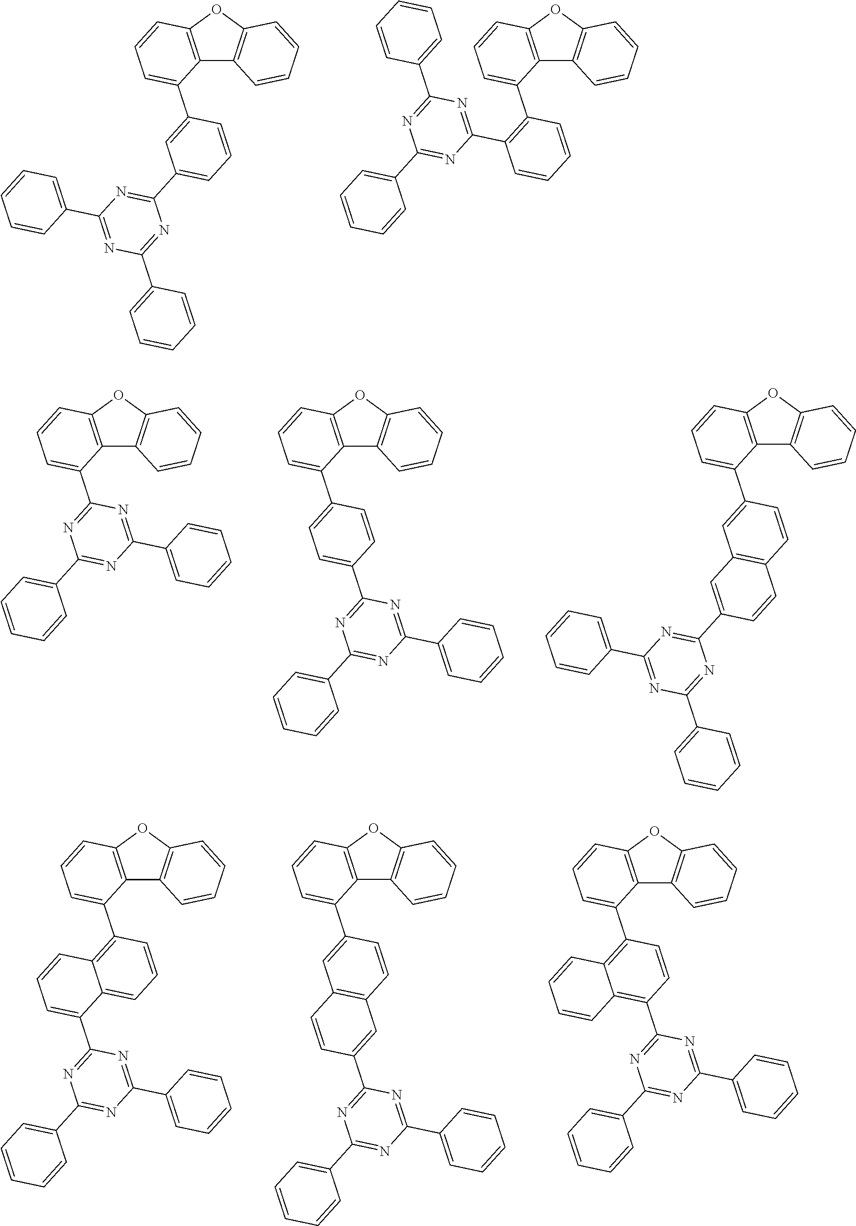

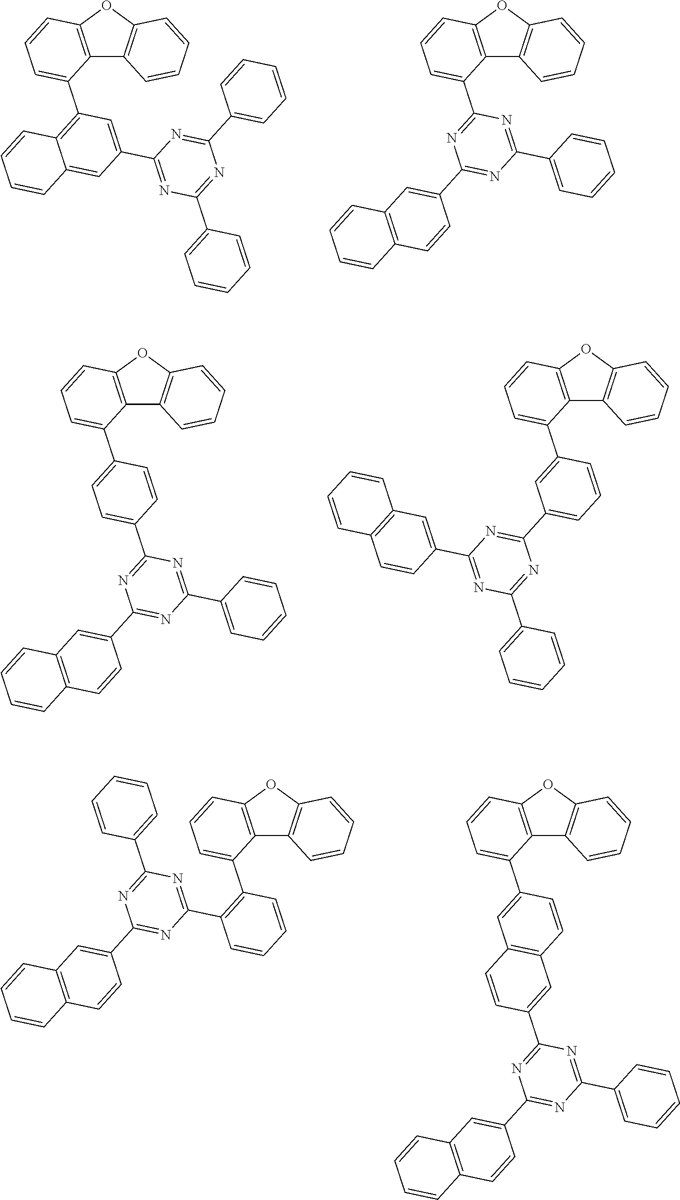 [SMILES: c1ccc(-c2nc(-c3ccccc3)nc(-c3cc(-c4cccc5oc6ccccc6c45)c4ccccc4c3)n2)cc1 ; c1ccc(-c2nc(-c3ccc4ccccc4c3)nc(-c3cccc4oc5ccccc5c34)n2)cc1 ; c1ccc(-c2nc(-c3ccc(-c4cccc5oc6ccccc6c45)cc3)nc(-c3ccc4ccccc4c3)n2)cc1 ; c1ccc(-c2nc(-c3cccc(-c4cccc5oc6ccccc6c45)c3)nc(-c3ccc4ccccc4c3)n2)cc1 ; c1ccc(-c2nc(-c3ccc4ccccc4c3)nc(-c3ccccc3-c3cccc4oc5ccccc5c34)n2)cc1 ; c1ccc(-c2nc(-c3ccc4ccccc4c3)nc(-c3ccc4cc(-c5cccc6oc7ccccc7c56)ccc4c3)n2)cc1]

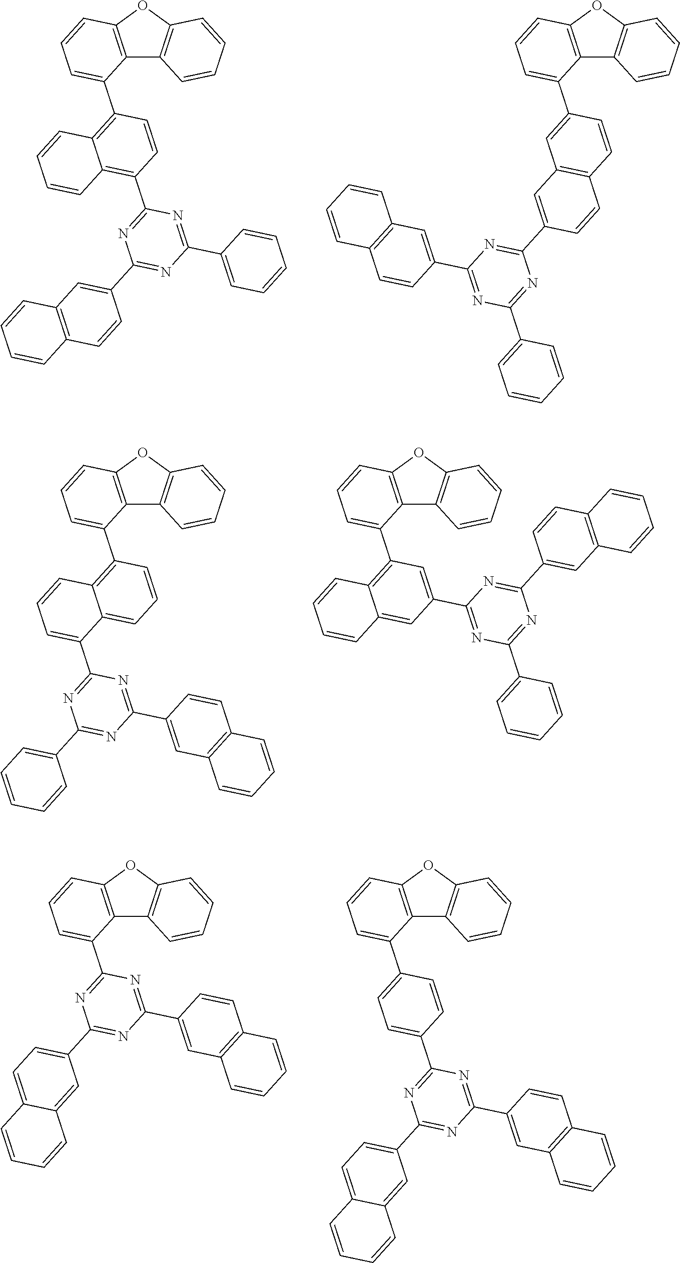 [SMILES: c1ccc(-c2nc(-c3ccc4ccccc4c3)nc(-c3ccc(-c4cccc5oc6ccccc6c45)c4ccccc34)n2)cc1 ; c1ccc(-c2nc(-c3ccc4ccccc4c3)nc(-c3ccc4ccc(-c5cccc6oc7ccccc7c56)cc4c3)n2)cc1 ; c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1 ; c1ccc2cc(-c3nc(-c4ccc5ccccc5c4)nc(-c4cccc5oc6ccccc6c45)n3)ccc2c1 ; c1ccc2cc(-c3nc(-c4ccc(-c5cccc6oc7ccccc7c56)cc4)nc(-c4ccc5ccccc5c4)n3)ccc2c1]

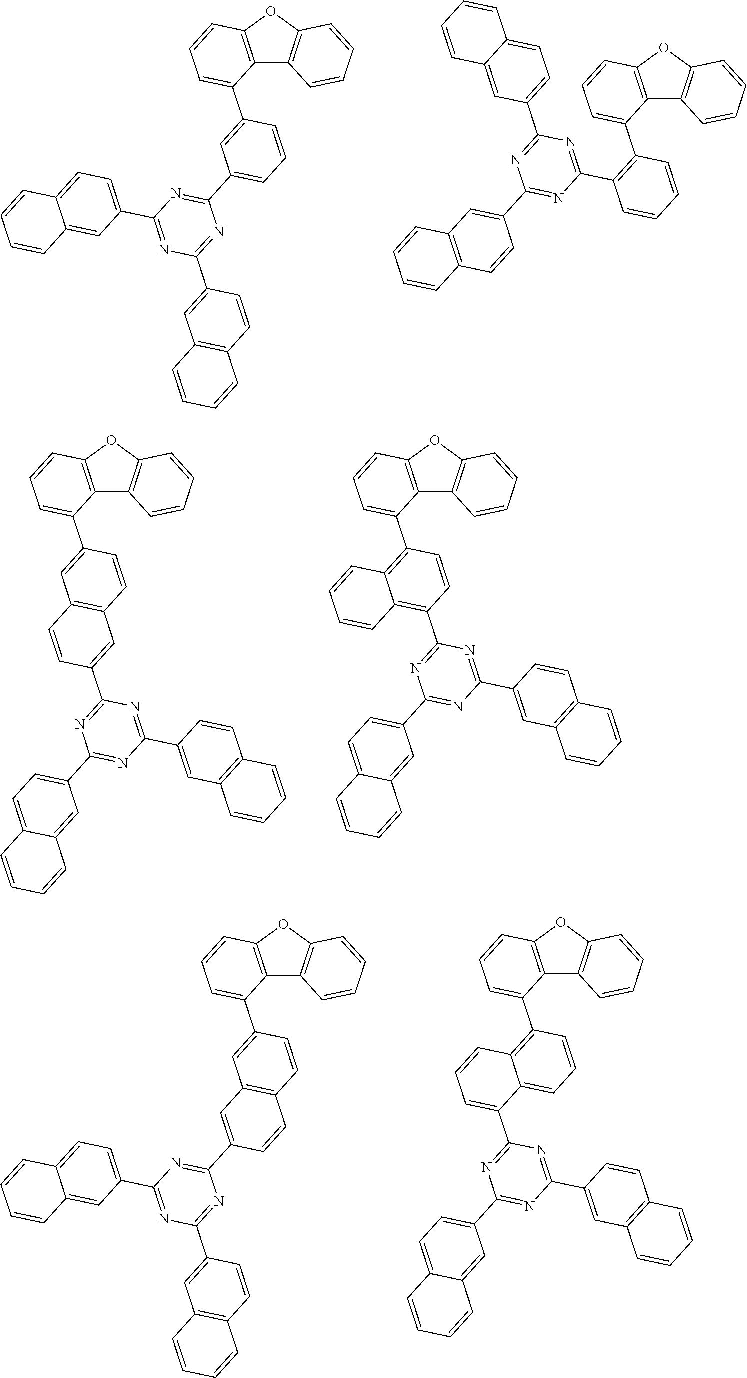 [SMILES: c1cc(-c2nc(-c3ccc4ccccc4c3)nc(-c3ccc4ccccc4c3)n2)cc(-c2cccc3oc4ccccc4c23)c1 ; c1ccc(-c2cccc3oc4ccccc4c23)c(-c2nc(-c3ccc4ccccc4c3)nc(-c3ccc4ccccc4c3)n2)c1 ; c1ccc2cc(-c3nc(-c4ccc5ccccc5c4)nc(-c4ccc5cc(-c6cccc7oc8ccccc8c67)ccc5c4)n3)ccc2c1 ; c1ccc2cc(-c3nc(-c4ccc5ccccc5c4)nc(-c4ccc(-c5cccc6oc7ccccc7c56)c5ccccc45)n3)ccc2c1 ; c1ccc2cc(-c3nc(-c4ccc5ccccc5c4)nc(-c4ccc5ccc(-c6cccc7oc8ccccc8c67)cc5c4)n3)ccc2c1 ; c1ccc2cc(-c3nc(-c4ccc5ccccc5c4)nc(-c4cccc5c(-c6cccc7oc8ccccc8c67)cccc45)n3)ccc2c1]

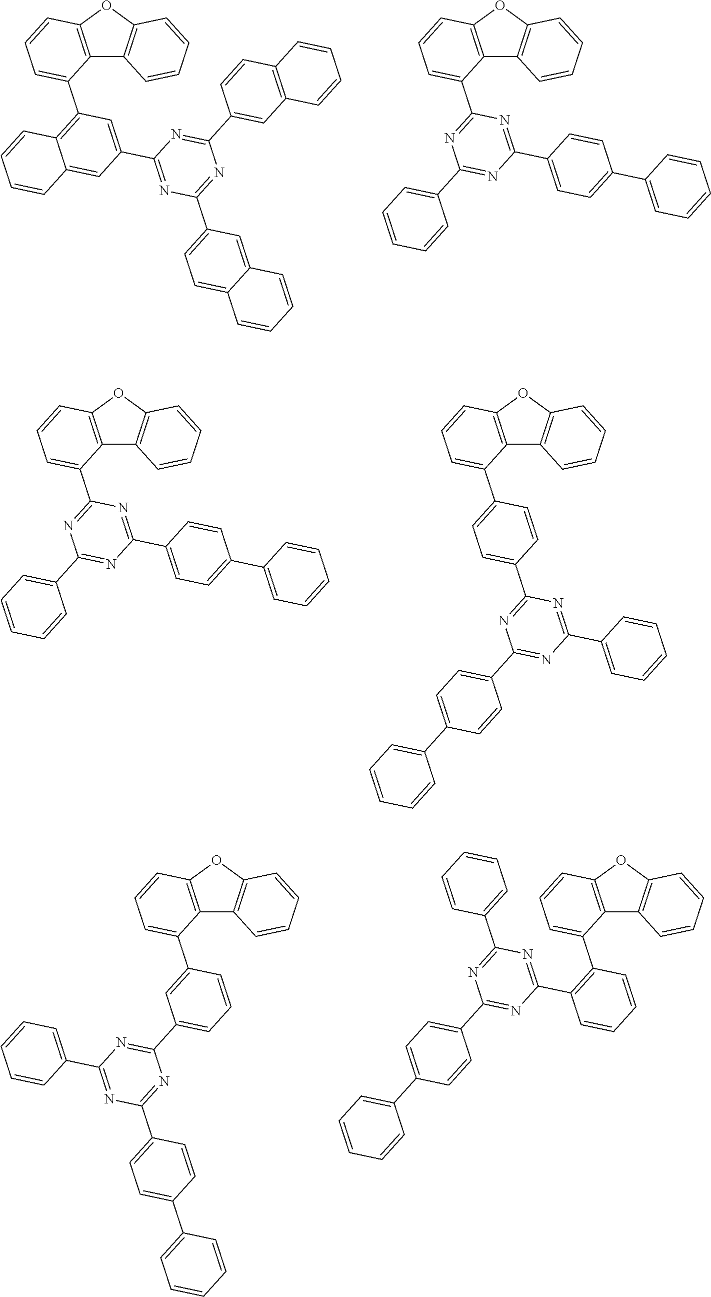 [SMILES: c1ccc2cc(-c3nc(-c4ccc5ccccc5c4)nc(-c4cc(-c5cccc6oc7ccccc7c56)c5ccccc5c4)n3)ccc2c1 ; c1ccc(-c2ccc(-c3nc(-c4ccccc4)nc(-c4cccc5oc6ccccc6c45)n3)cc2)cc1 ; c1ccc(-c2ccc(-c3nc(-c4ccccc4)nc(-c4cccc5oc6ccccc6c45)n3)cc2)cc1 ; c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4cccc(-c5cccc6oc7ccccc7c56)c4)n3)cc2)cc1 ; c1ccc(-c2ccc(-c3nc(-c4ccccc4)nc(-c4ccccc4-c4cccc5oc6ccccc6c45)n3)cc2)cc1]

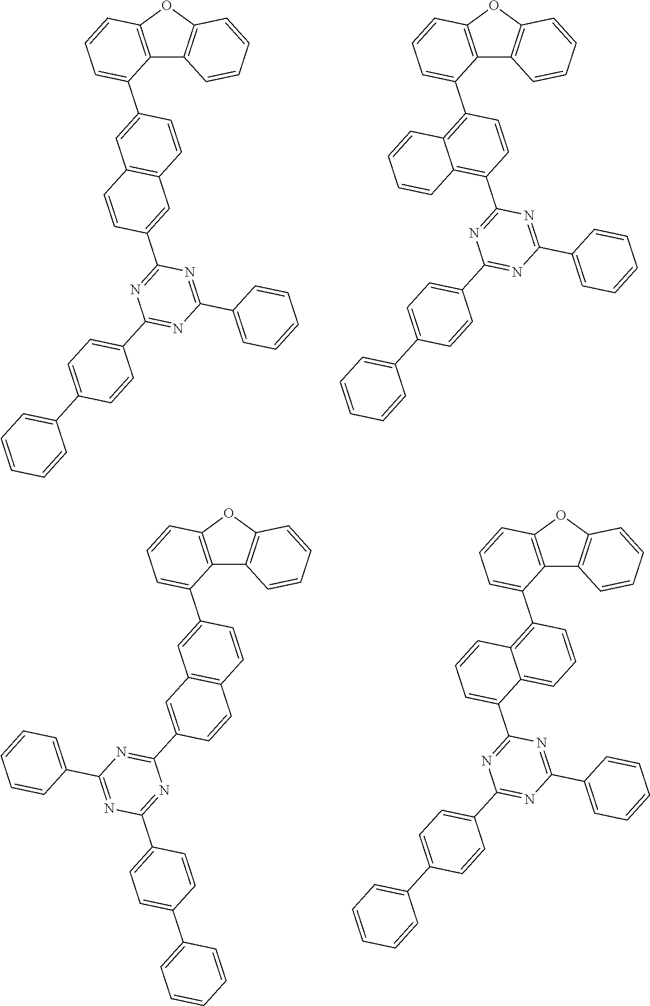 [SMILES: c1ccc(-c2ccc(-c3nc(-c4ccccc4)nc(-c4ccc5cc(-c6cccc7oc8ccccc8c67)ccc5c4)n3)cc2)cc1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2ccc(-c3nc(-c4ccccc4)nc(-c4ccc5ccc(-c6cccc7oc8ccccc8c67)cc5c4)n3)cc2)cc1 ; c1ccc(-c2ccc(-c3nc(-c4ccccc4)nc(-c4cccc5c(-c6cccc7oc8ccccc8c67)cccc45)n3)cc2)cc1]

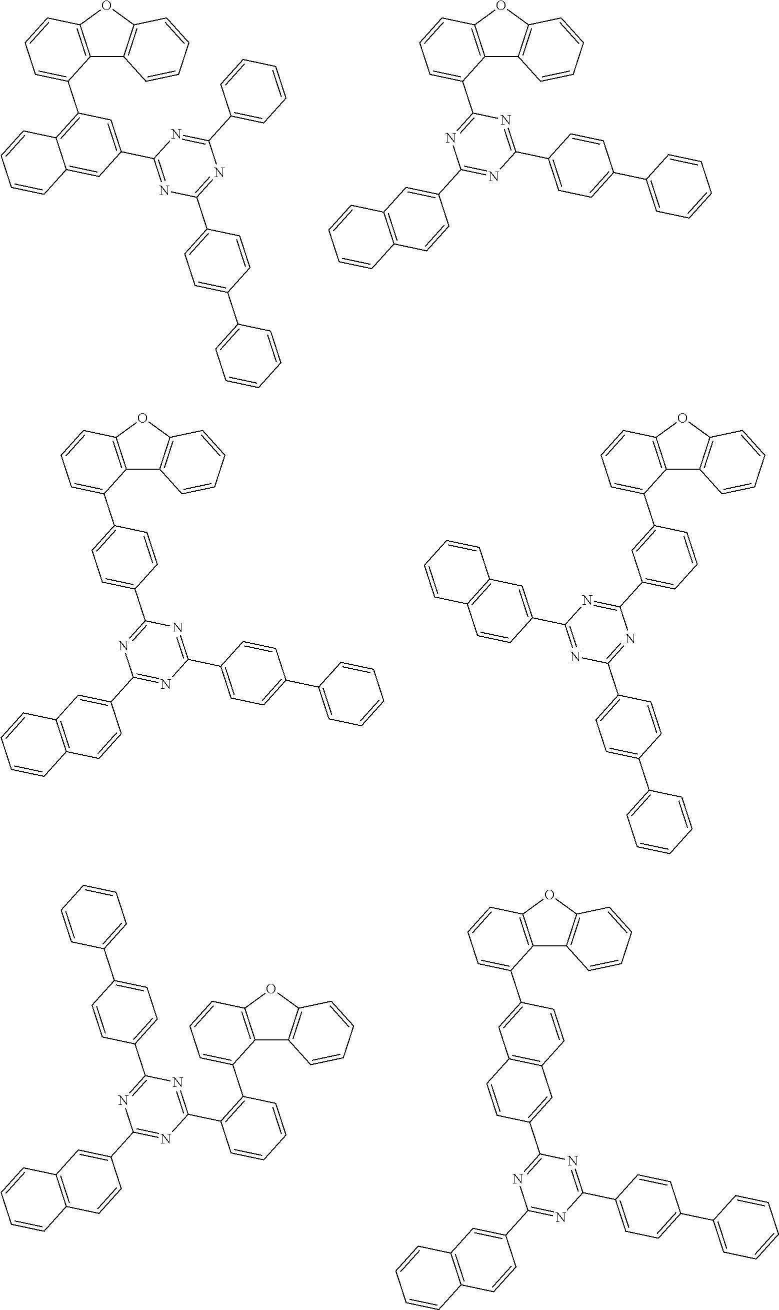 [SMILES: c1ccc(-c2ccc(-c3nc(-c4ccccc4)nc(-c4cc(-c5cccc6oc7ccccc7c56)c5ccccc5c4)n3)cc2)cc1 ; c1ccc(-c2ccc(-c3nc(-c4ccc5ccccc5c4)nc(-c4cccc5oc6ccccc6c45)n3)cc2)cc1 ; c1ccc(-c2ccc(-c3nc(-c4ccc(-c5cccc6oc7ccccc7c56)cc4)nc(-c4ccc5ccccc5c4)n3)cc2)cc1 ; c1ccc(-c2ccc(-c3nc(-c4cccc(-c5cccc6oc7ccccc7c56)c4)nc(-c4ccc5ccccc5c4)n3)cc2)cc1 ; c1ccc(-c2ccc(-c3nc(-c4ccc5ccccc5c4)nc(-c4ccccc4-c4cccc5oc6ccccc6c45)n3)cc2)cc1 ; c1ccc(-c2ccc(-c3nc(-c4ccc5ccccc5c4)nc(-c4ccc5cc(-c6cccc7oc8ccccc8c67)ccc5c4)n3)cc2)cc1]

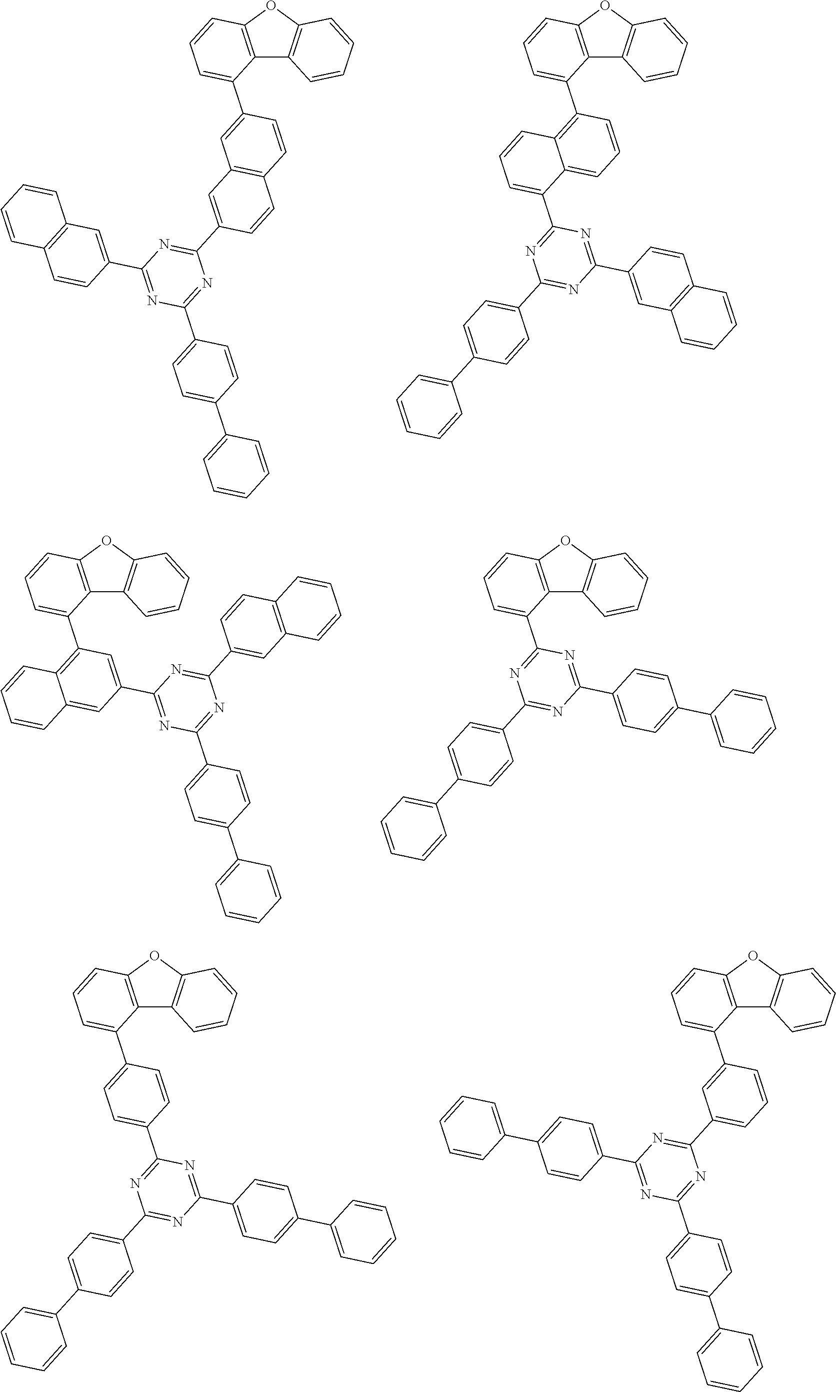

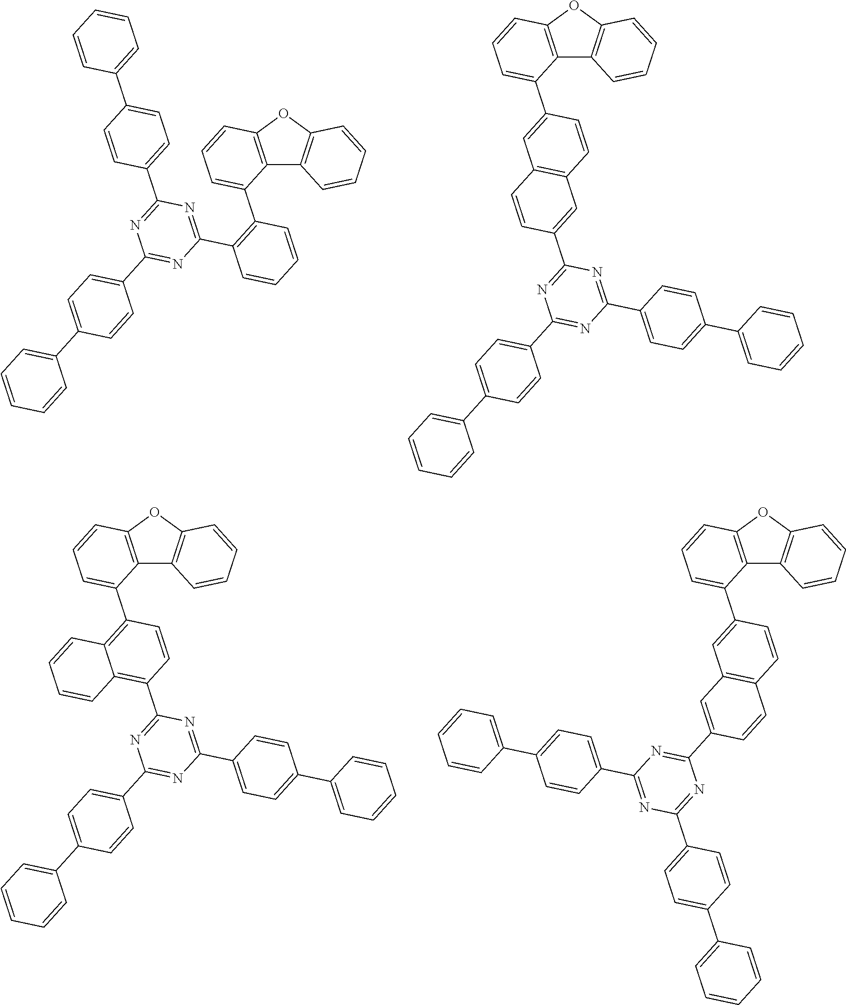 [SMILES: c1ccc(-c2ccc(-c3nc(-c4ccc(-c5ccccc5)cc4)nc(-c4ccccc4-c4cccc5oc6ccccc6c45)n3)cc2)cc1 ; c1ccc(-c2ccc(-c3nc(-c4ccc(-c5ccccc5)cc4)nc(-c4ccc5cc(-c6cccc7oc8ccccc8c67)ccc5c4)n3)cc2)cc1 ; c1ccc(-c2ccc(-c3nc(-c4ccc(-c5ccccc5)cc4)nc(-c4ccc(-c5cccc6oc7ccccc7c56)c5ccccc45)n3)cc2)cc1 ; c1ccc(-c2ccc(-c3nc(-c4ccc(-c5ccccc5)cc4)nc(-c4ccc5ccc(-c6cccc7oc8ccccc8c67)cc5c4)n3)cc2)cc1]

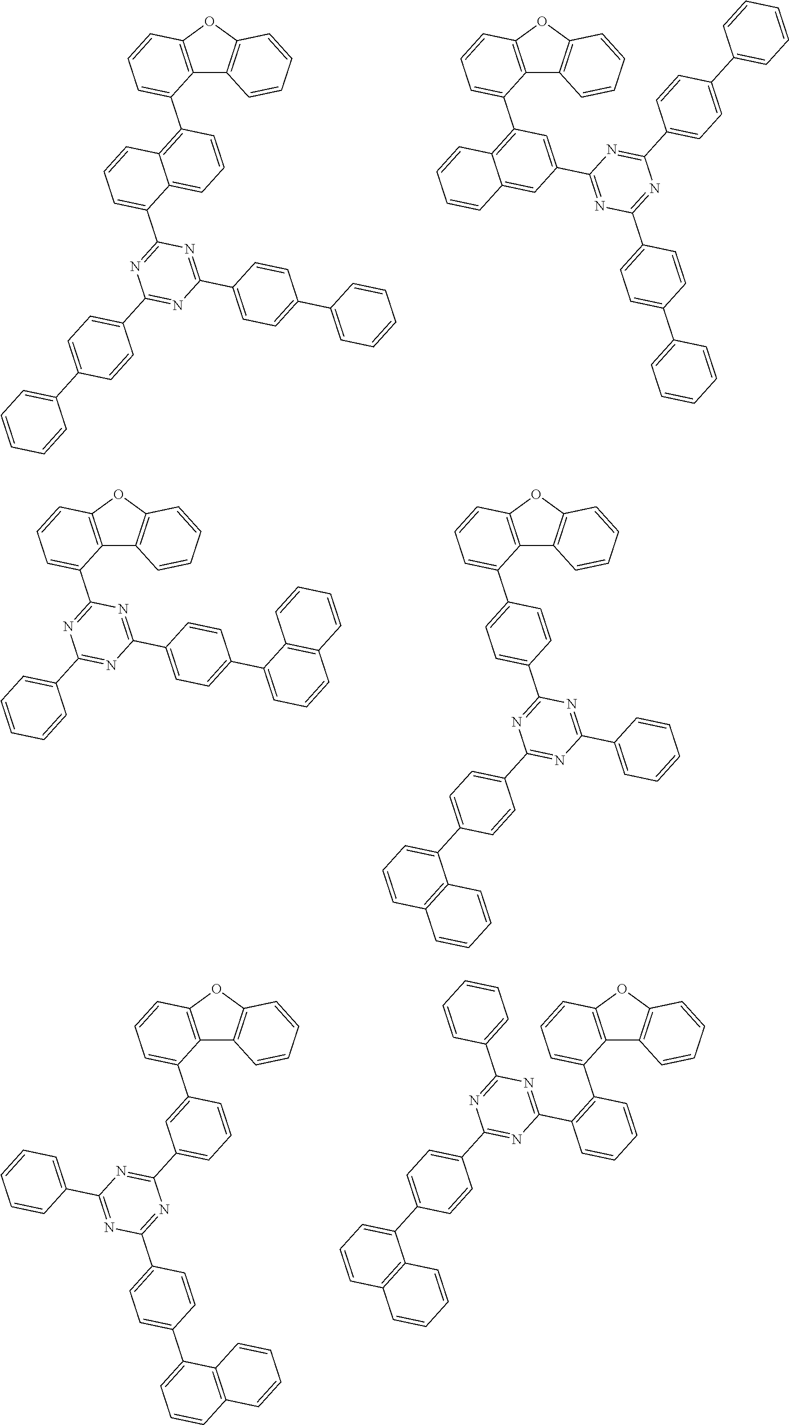

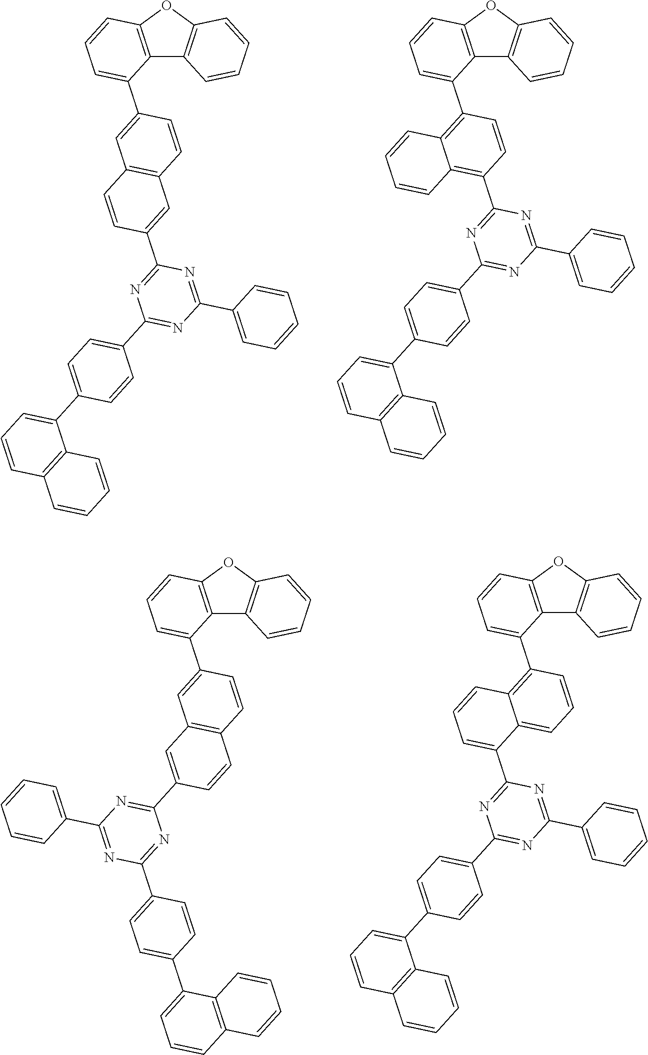 [SMILES: c1ccc(-c2nc(-c3ccc(-c4cccc5ccccc45)cc3)nc(-c3ccc4cc(-c5cccc6oc7ccccc7c56)ccc4c3)n2)cc1 ; c1ccc(-c2nc(-c3ccc(-c4cccc5ccccc45)cc3)nc(-c3ccc(-c4cccc5oc6ccccc6c45)c4ccccc34)n2)cc1 ; c1ccc(-c2nc(-c3ccc(-c4cccc5ccccc45)cc3)nc(-c3ccc4ccc(-c5cccc6oc7ccccc7c56)cc4c3)n2)cc1 ; c1ccc(-c2nc(-c3ccc(-c4cccc5ccccc45)cc3)nc(-c3cccc4c(-c5cccc6oc7ccccc7c56)cccc34)n2)cc1]

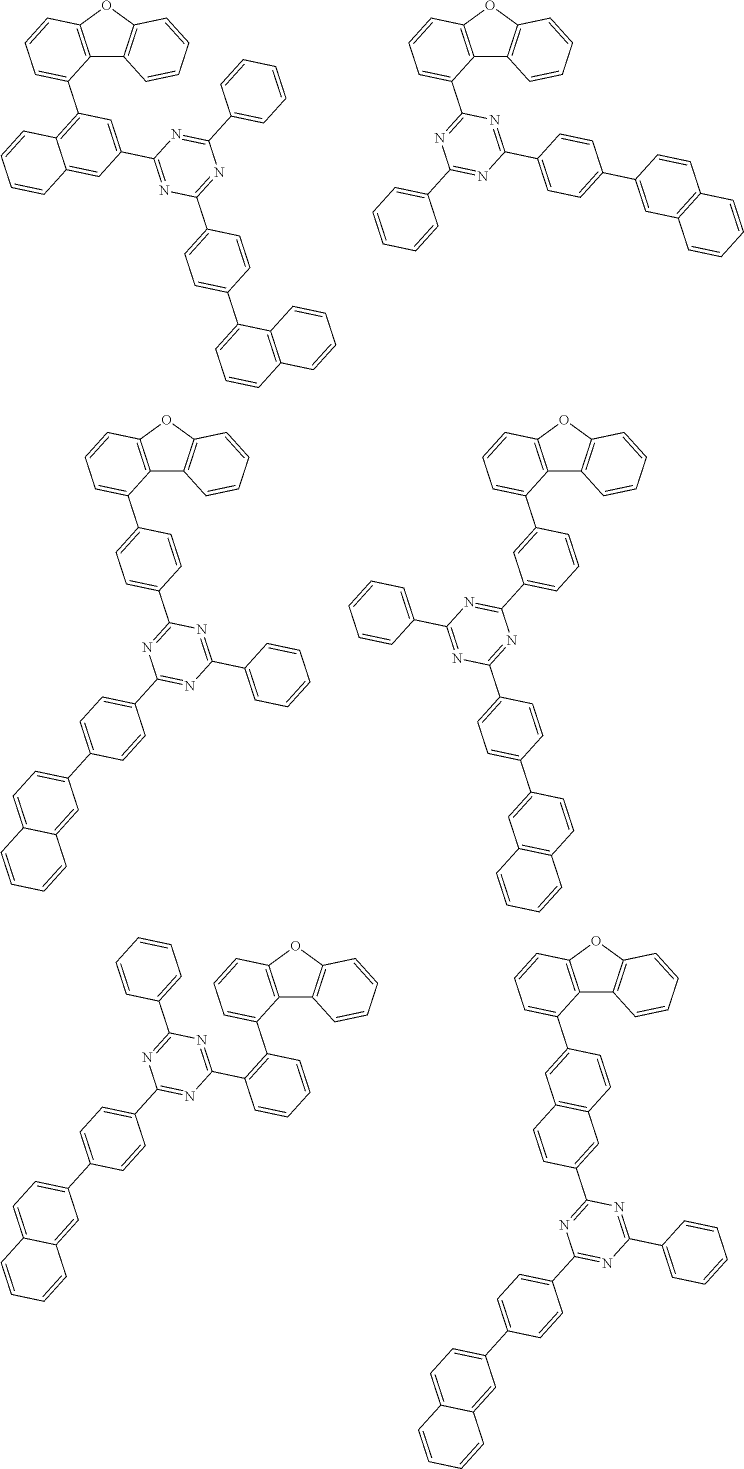

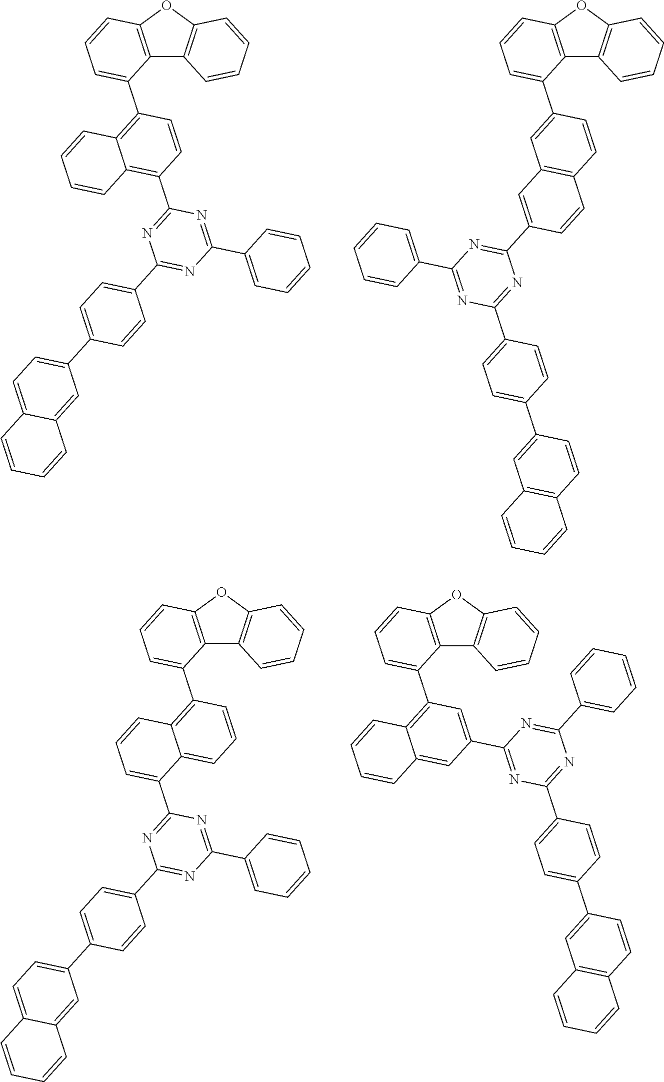 [SMILES: c1ccc(-c2nc(-c3ccc(-c4ccc5ccccc5c4)cc3)nc(-c3ccc(-c4cccc5oc6ccccc6c45)c4ccccc34)n2)cc1 ; c1ccc(-c2nc(-c3ccc(-c4ccc5ccccc5c4)cc3)nc(-c3ccc4ccc(-c5cccc6oc7ccccc7c56)cc4c3)n2)cc1 ; c1ccc(-c2nc(-c3ccc(-c4ccc5ccccc5c4)cc3)nc(-c3cccc4c(-c5cccc6oc7ccccc7c56)cccc34)n2)cc1 ; c1ccc(-c2nc(-c3ccc(-c4ccc5ccccc5c4)cc3)nc(-c3cc(-c4cccc5oc6ccccc6c45)c4ccccc4c3)n2)cc1]

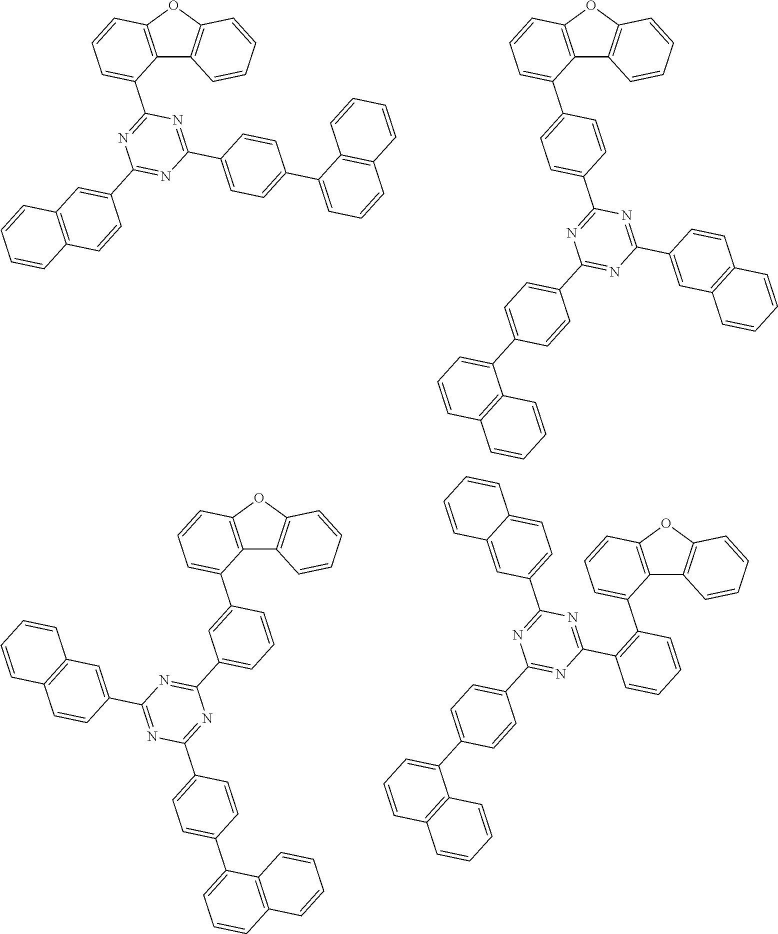

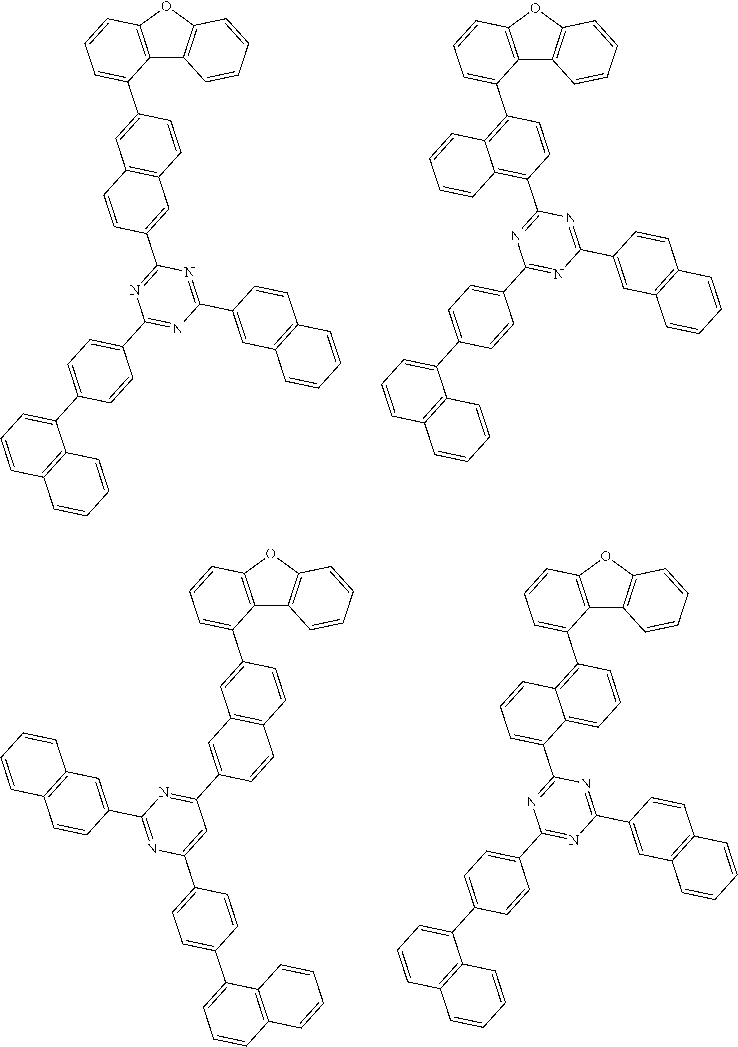

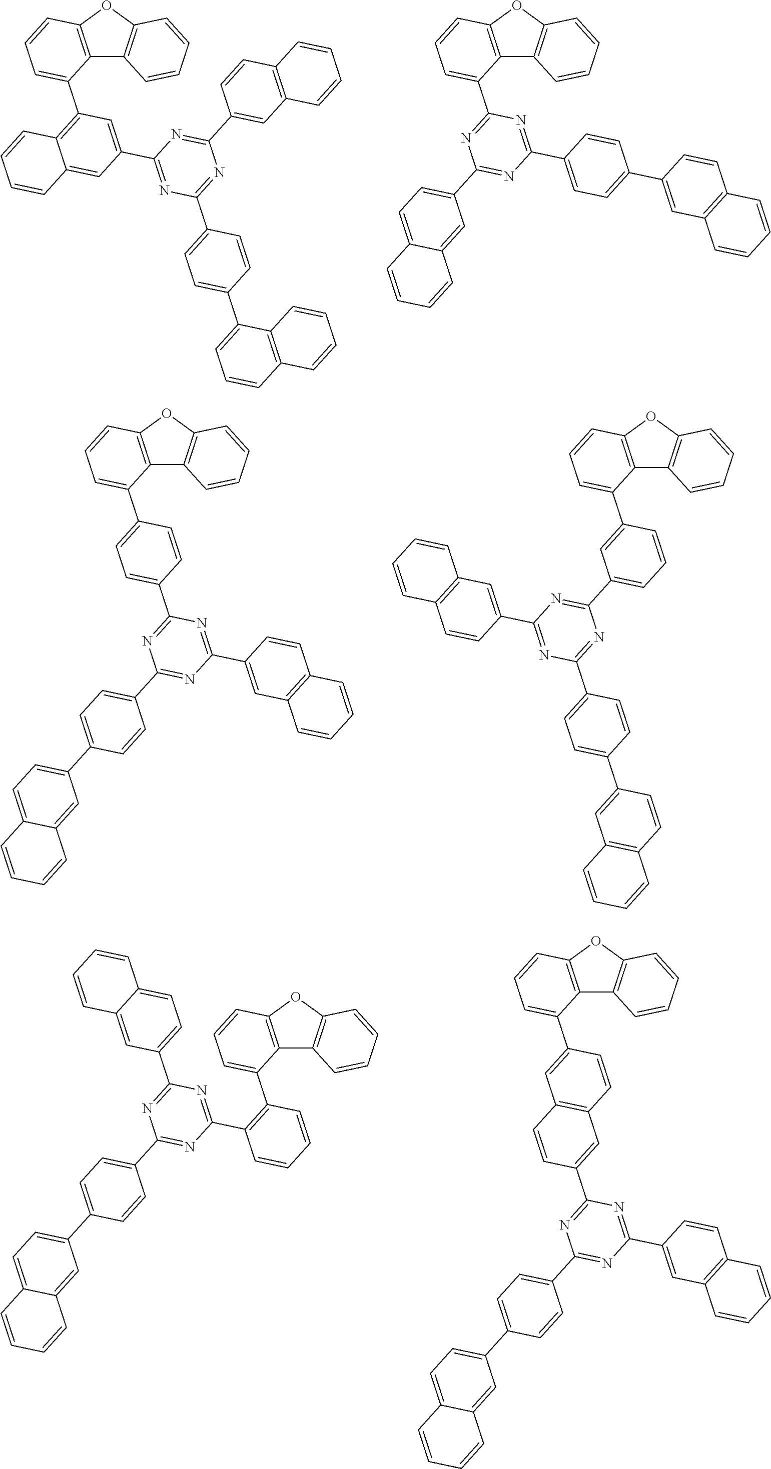

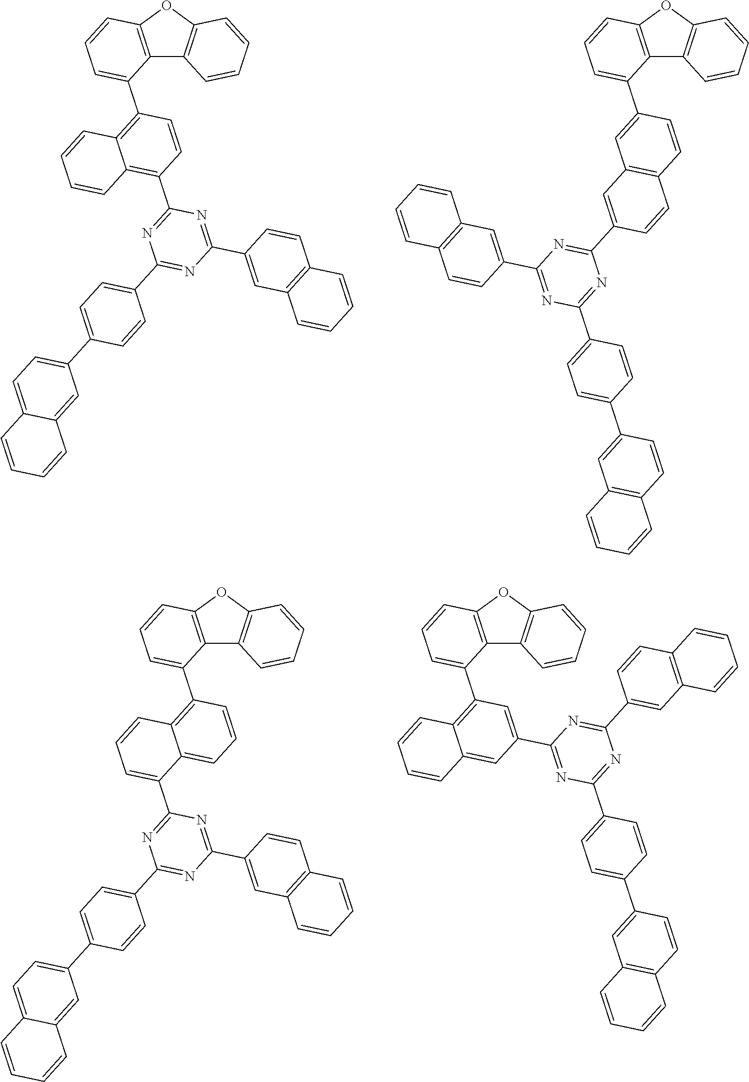 [SMILES: c1ccc2cc(-c3ccc(-c4nc(-c5ccc6ccccc6c5)nc(-c5ccc(-c6cccc7oc8ccccc8c67)c6ccccc56)n4)cc3)ccc2c1 ; c1ccc2cc(-c3ccc(-c4nc(-c5ccc6ccccc6c5)nc(-c5ccc6ccc(-c7cccc8oc9ccccc9c78)cc6c5)n4)cc3)ccc2c1 ; c1ccc2cc(-c3ccc(-c4nc(-c5ccc6ccccc6c5)nc(-c5cccc6c(-c7cccc8oc9ccccc9c78)cccc56)n4)cc3)ccc2c1 ; c1ccc2cc(-c3ccc(-c4nc(-c5ccc6ccccc6c5)nc(-c5cc(-c6cccc7oc8ccccc8c67)c6ccccc6c5)n4)cc3)ccc2c1]

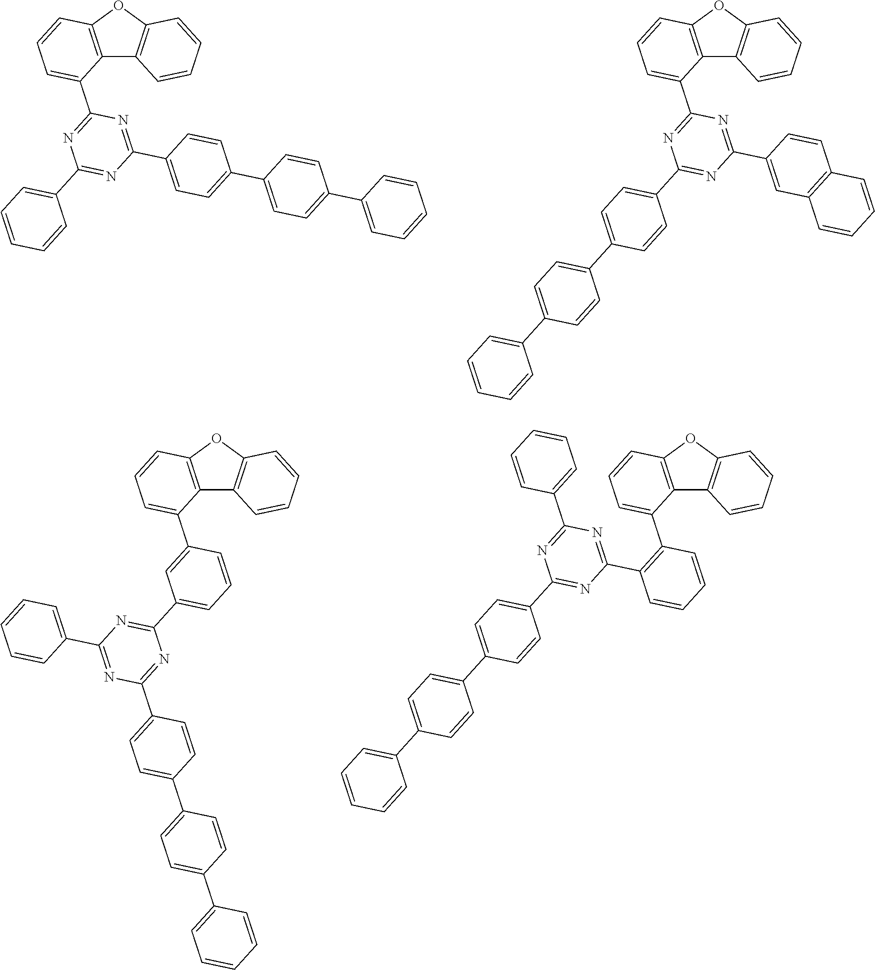

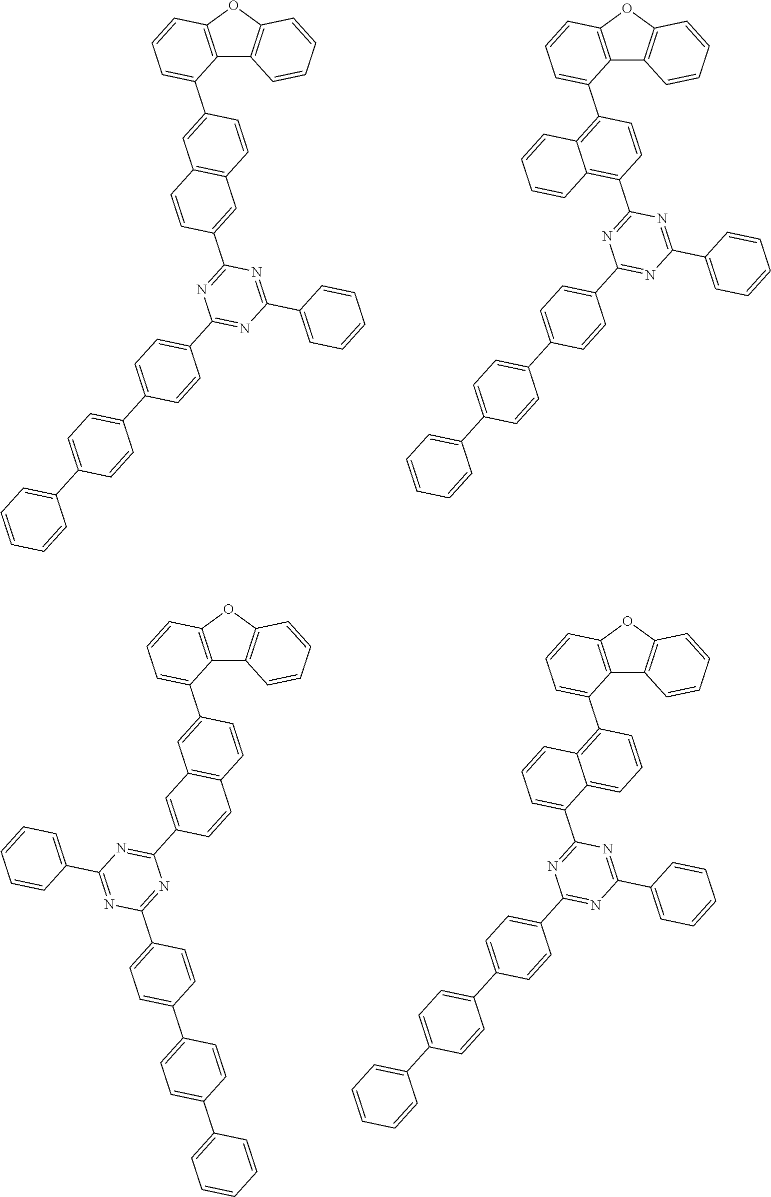 [SMILES: c1ccc(-c2ccc(-c3ccc(-c4nc(-c5ccccc5)nc(-c5ccc6cc(-c7cccc8oc9ccccc9c78)ccc6c5)n4)cc3)cc2)cc1 ; c1ccc(-c2ccc(-c3ccc(-c4nc(-c5ccccc5)nc(-c5ccc(-c6cccc7oc8ccccc8c67)c6ccccc56)n4)cc3)cc2)cc1 ; c1ccc(-c2ccc(-c3ccc(-c4nc(-c5ccccc5)nc(-c5ccc6ccc(-c7cccc8oc9ccccc9c78)cc6c5)n4)cc3)cc2)cc1 ; c1ccc(-c2ccc(-c3ccc(-c4nc(-c5ccccc5)nc(-c5cccc6c(-c7cccc8oc9ccccc9c78)cccc56)n4)cc3)cc2)cc1]

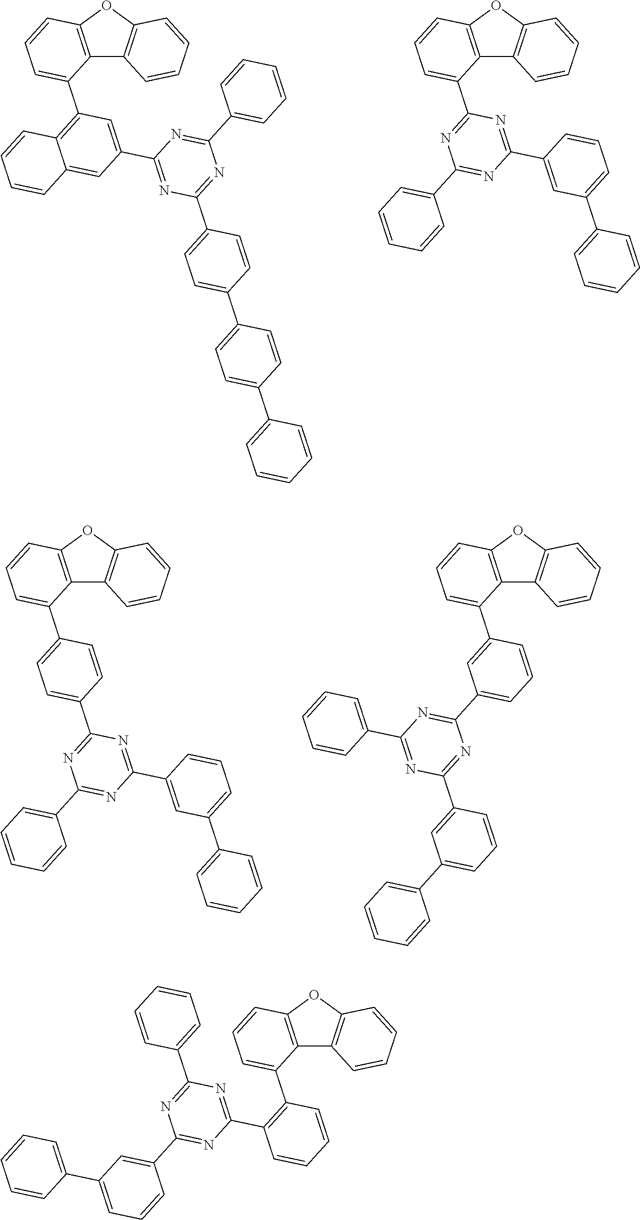 [SMILES: c1ccc(-c2ccc(-c3ccc(-c4nc(-c5ccccc5)nc(-c5cc(-c6cccc7oc8ccccc8c67)c6ccccc6c5)n4)cc3)cc2)cc1 ; c1ccc(-c2cccc(-c3nc(-c4ccccc4)nc(-c4cccc5oc6ccccc6c45)n3)c2)cc1 ; c1ccc(-c2cccc(-c3nc(-c4ccccc4)nc(-c4ccc(-c5cccc6oc7ccccc7c56)cc4)n3)c2)cc1 ; c1ccc(-c2cccc(-c3nc(-c4ccccc4)nc(-c4cccc(-c5cccc6oc7ccccc7c56)c4)n3)c2)cc1 ; c1ccc(-c2cccc(-c3nc(-c4ccccc4)nc(-c4ccccc4-c4cccc5oc6ccccc6c45)n3)c2)cc1]

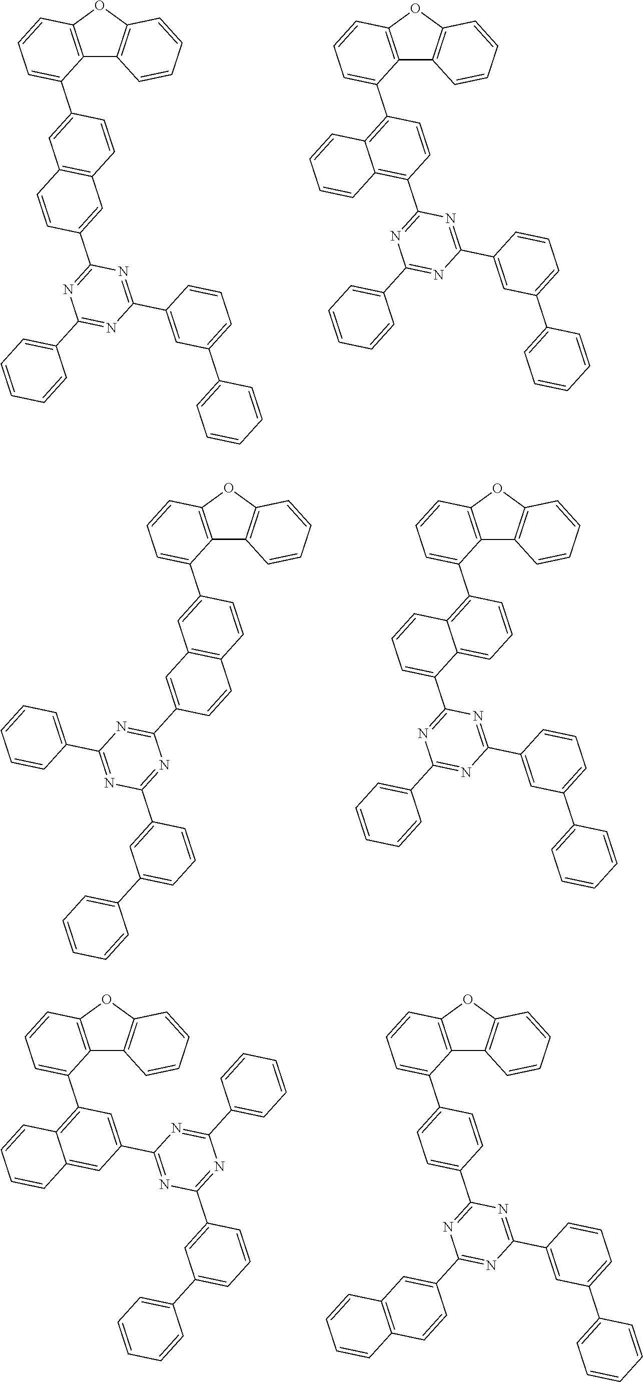

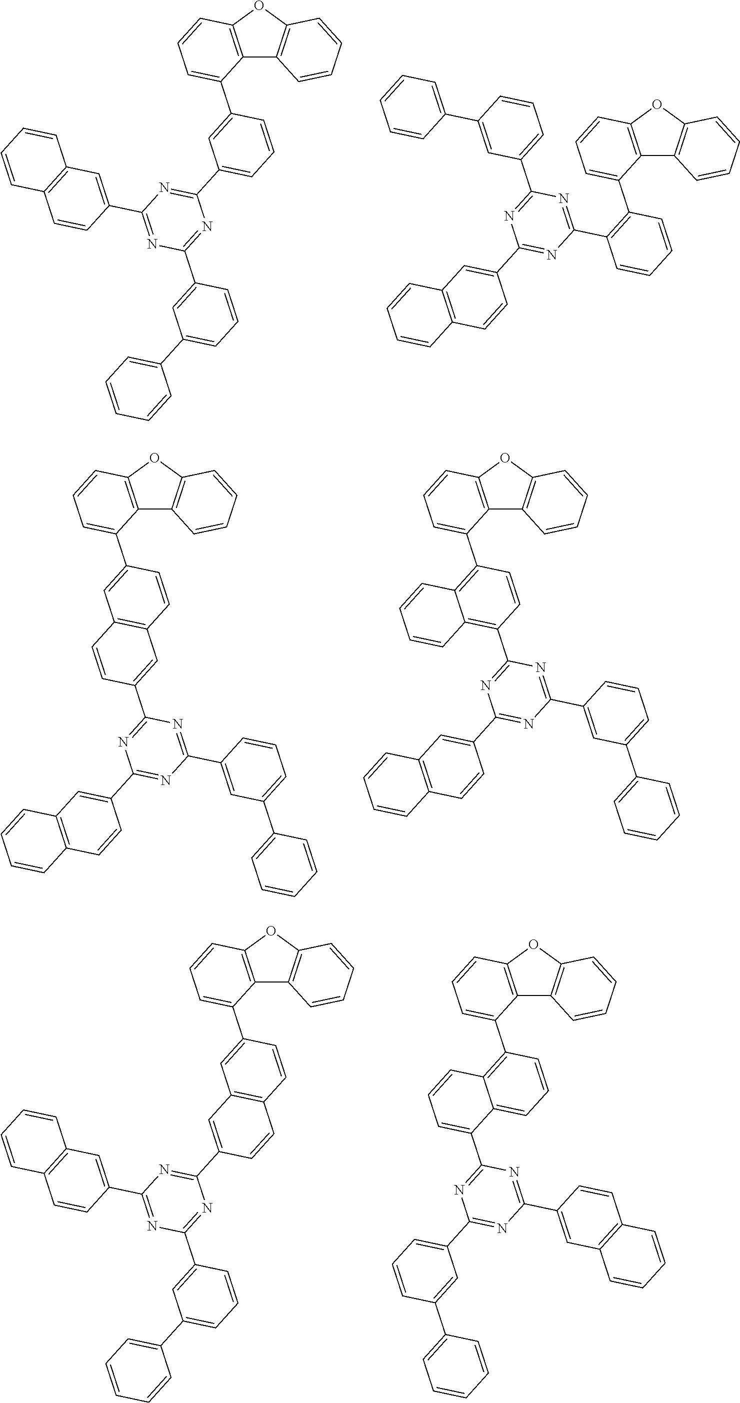

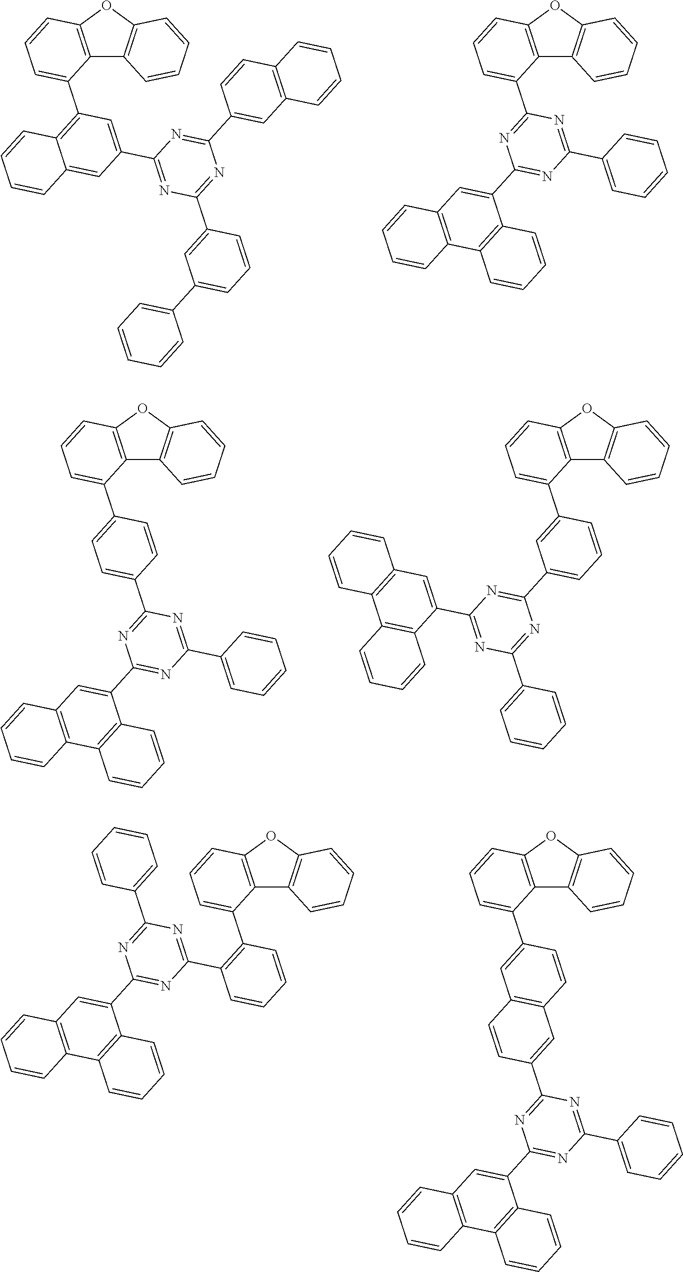

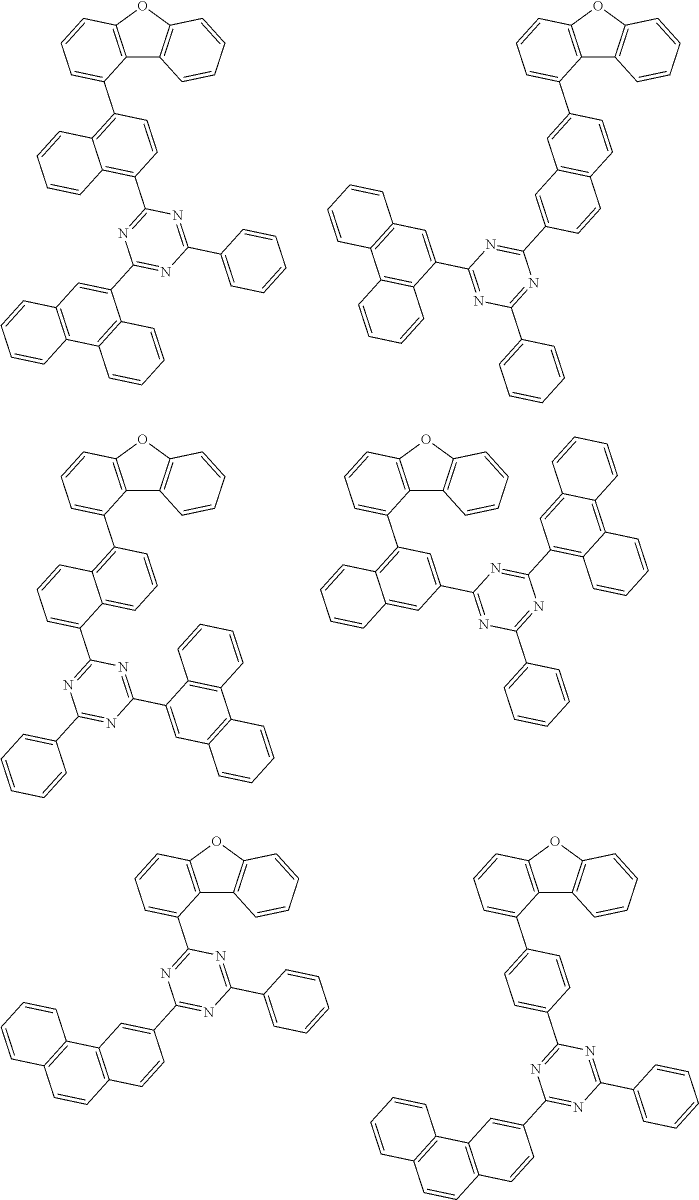 [SMILES: c1ccc(-c2nc(-c3ccc(-c4cccc5oc6ccccc6c45)c4ccccc34)nc(-c3cc4ccccc4c4ccccc34)n2)cc1 ; c1ccc(-c2nc(-c3ccc4ccc(-c5cccc6oc7ccccc7c56)cc4c3)nc(-c3cc4ccccc4c4ccccc34)n2)cc1 ; c1ccc(-c2nc(-c3cccc4c(-c5cccc6oc7ccccc7c56)cccc34)nc(-c3cc4ccccc4c4ccccc34)n2)cc1 ; c1ccc(-c2nc(-c3cc(-c4cccc5oc6ccccc6c45)c4ccccc4c3)nc(-c3cc4ccccc4c4ccccc34)n2)cc1 ; c1ccc(-c2nc(-c3ccc4ccc5ccccc5c4c3)nc(-c3cccc4oc5ccccc5c34)n2)cc1 ; c1ccc(-c2nc(-c3ccc(-c4cccc5oc6ccccc6c45)cc3)nc(-c3ccc4ccc5ccccc5c4c3)n2)cc1]

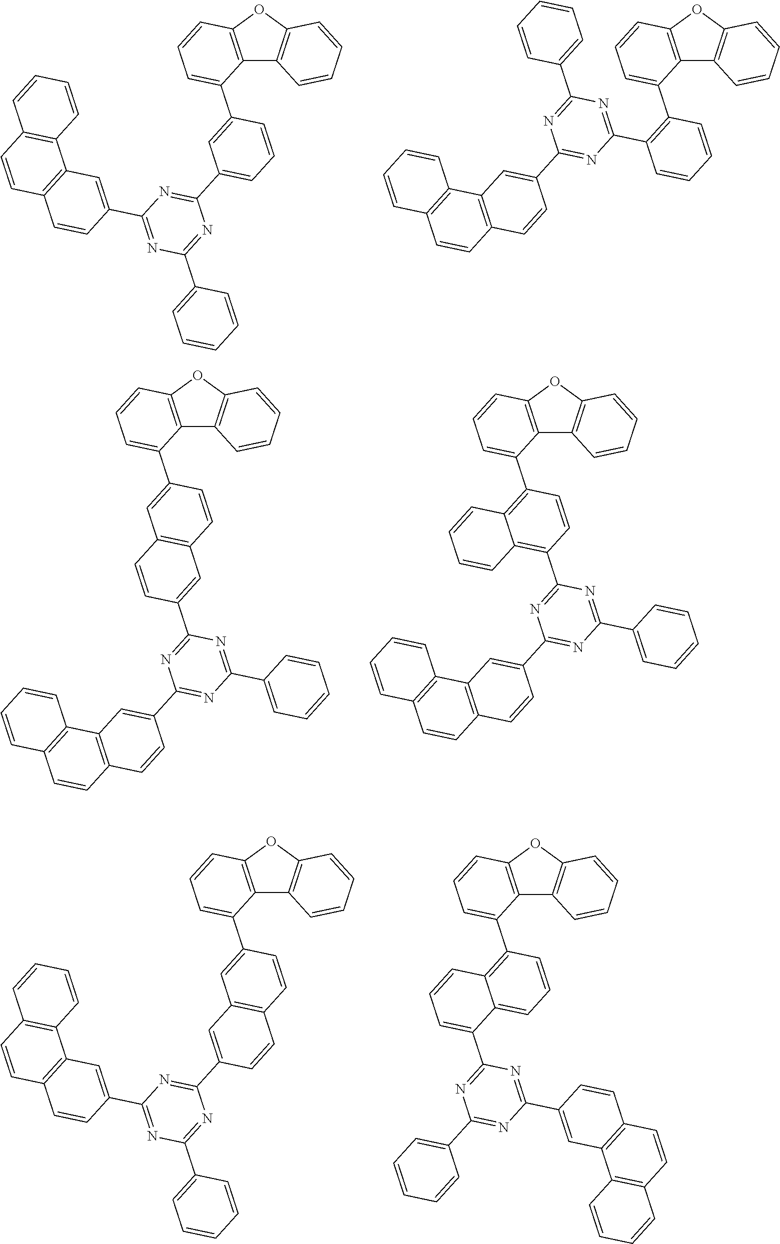

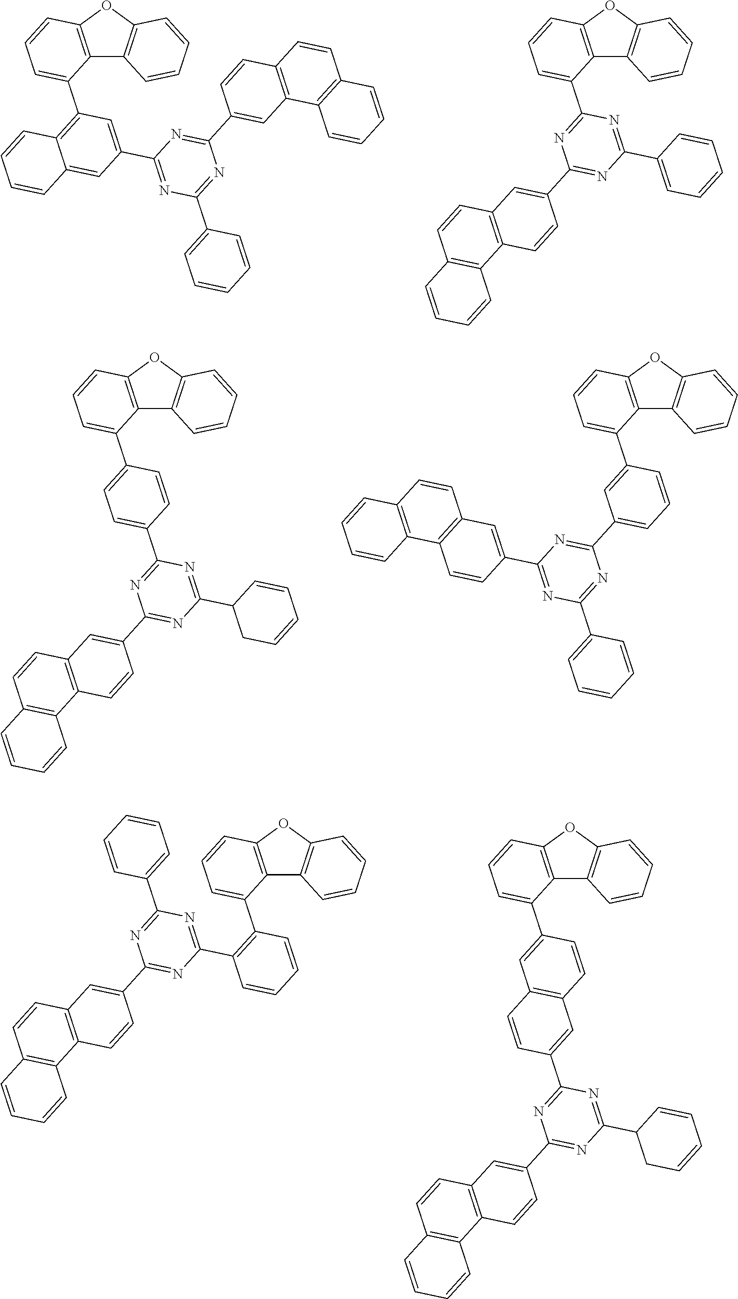 [SMILES: c1ccc(-c2nc(-c3cc(-c4cccc5oc6ccccc6c45)c4ccccc4c3)nc(-c3ccc4ccc5ccccc5c4c3)n2)cc1 ; c1ccc(-c2nc(-c3ccc4c(ccc5ccccc54)c3)nc(-c3cccc4oc5ccccc5c34)n2)cc1 ; C1=CCC(c2nc(-c3ccc(-c4cccc5oc6ccccc6c45)cc3)nc(-c3ccc4c(ccc5ccccc54)c3)n2)C=C1 ; c1ccc(-c2nc(-c3cccc(-c4cccc5oc6ccccc6c45)c3)nc(-c3ccc4c(ccc5ccccc54)c3)n2)cc1 ; c1ccc(-c2nc(-c3ccc4c(ccc5ccccc54)c3)nc(-c3ccccc3-c3cccc4oc5ccccc5c34)n2)cc1 ; C1=CCC(c2nc(-c3ccc4cc(-c5cccc6oc7ccccc7c56)ccc4c3)nc(-c3ccc4c(ccc5ccccc54)c3)n2)C=C1]

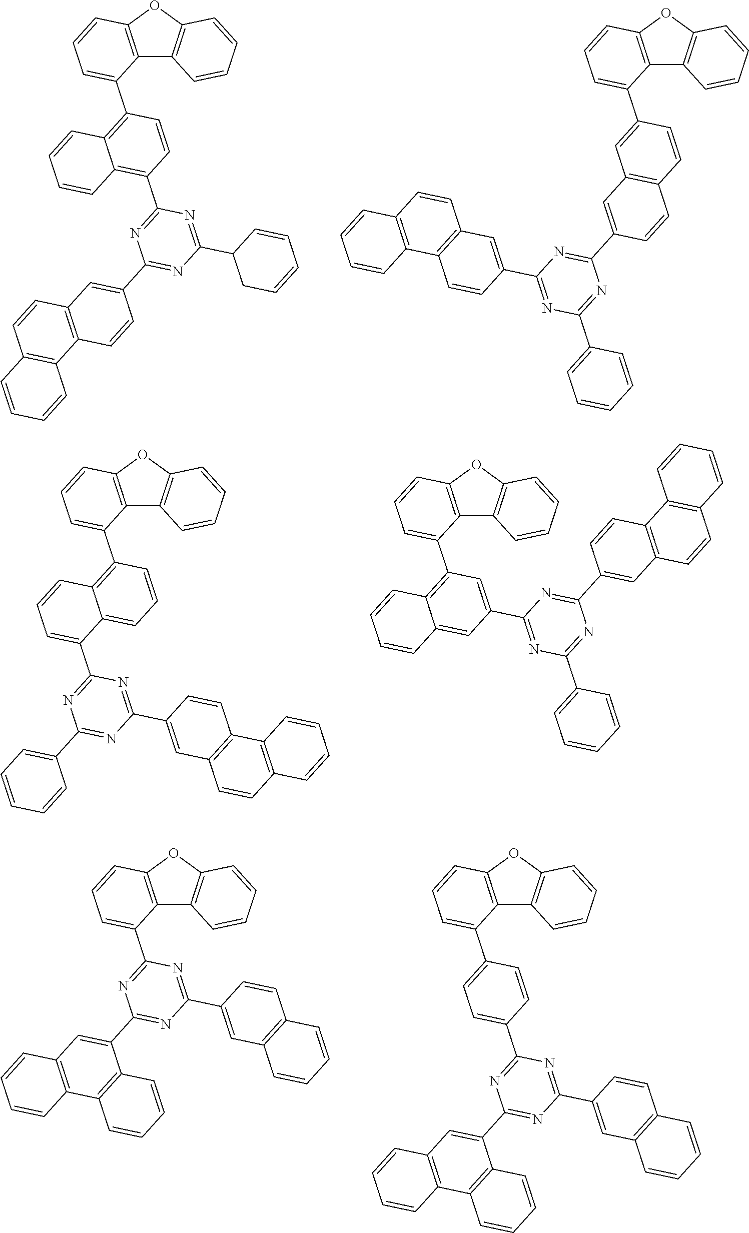 [SMILES: C1=CCC(c2nc(-c3ccc4c(ccc5ccccc54)c3)nc(-c3ccc(-c4cccc5oc6ccccc6c45)c4ccccc34)n2)C=C1 ; c1ccc(-c2nc(-c3ccc4ccc(-c5cccc6oc7ccccc7c56)cc4c3)nc(-c3ccc4c(ccc5ccccc54)c3)n2)cc1 ; c1ccc(-c2nc(-c3ccc4c(ccc5ccccc54)c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4c(ccc5ccccc54)c3)n2)cc1 ; c1ccc2cc(-c3nc(-c4cc5ccccc5c5ccccc45)nc(-c4cccc5oc6ccccc6c45)n3)ccc2c1 ; c1ccc2cc(-c3nc(-c4ccc(-c5cccc6oc7ccccc7c56)cc4)nc(-c4cc5ccccc5c5ccccc45)n3)ccc2c1]

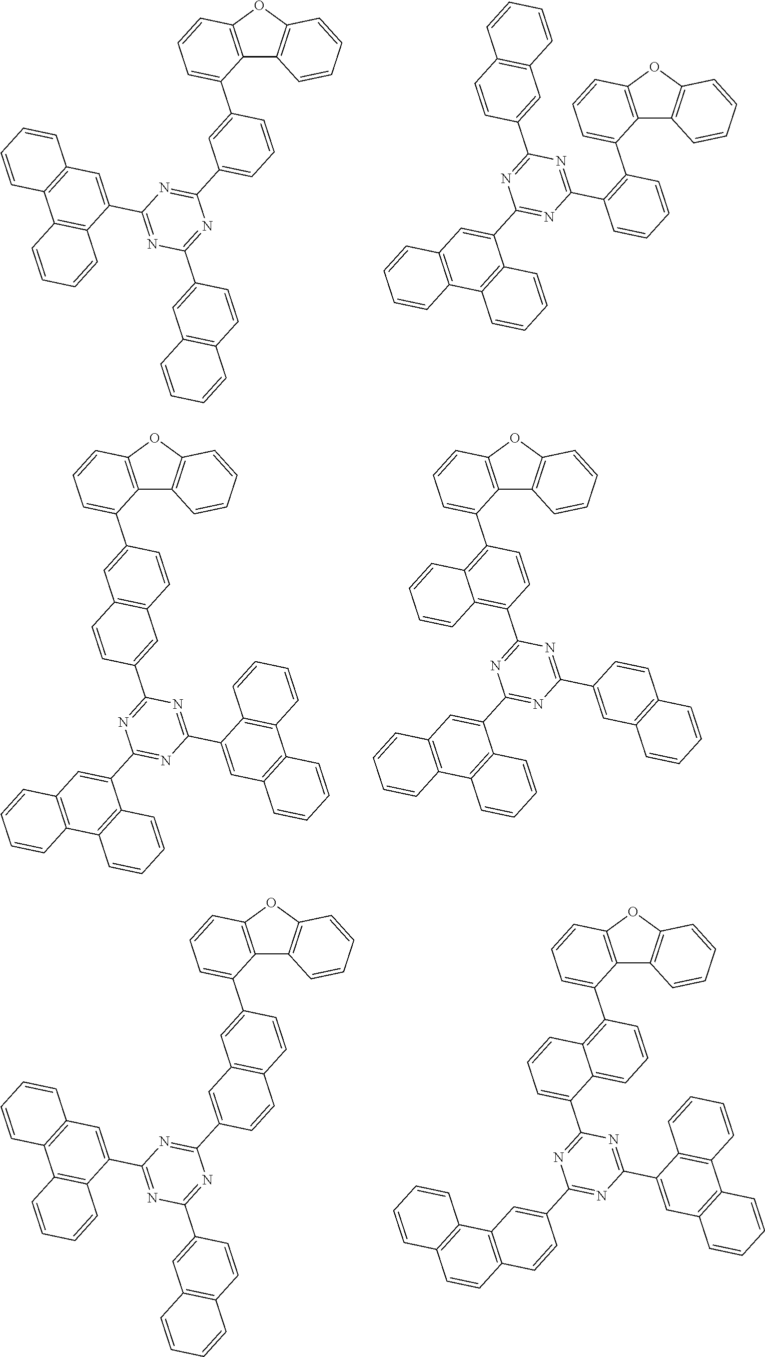

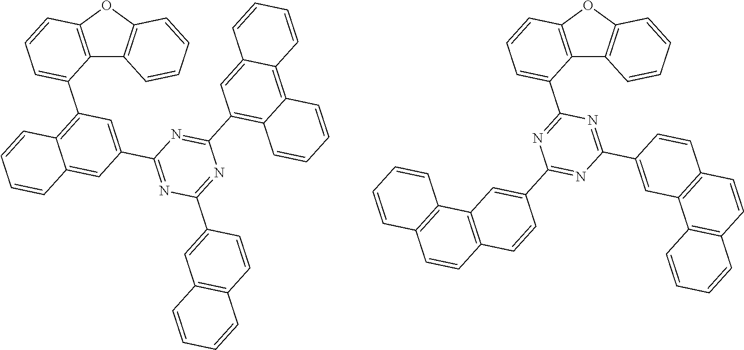 [SMILES: c1ccc2cc(-c3nc(-c4cc(-c5cccc6oc7ccccc7c56)c5ccccc5c4)nc(-c4cc5ccccc5c5ccccc45)n3)ccc2c1 ; c1ccc2c(c1)ccc1ccc(-c3nc(-c4ccc5ccc6ccccc6c5c4)nc(-c4cccc5oc6ccccc6c45)n3)cc12]

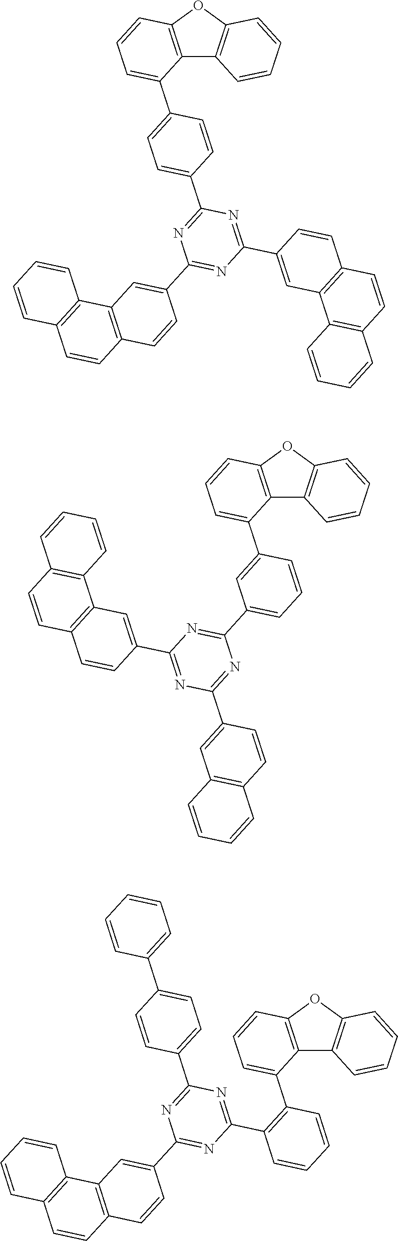 [SMILES: c1ccc2c(c1)ccc1ccc(-c3nc(-c4ccc(-c5cccc6oc7ccccc7c56)cc4)nc(-c4ccc5ccc6ccccc6c5c4)n3)cc12 ; c1cc(-c2nc(-c3ccc4ccccc4c3)nc(-c3ccc4ccc5ccccc5c4c3)n2)cc(-c2cccc3oc4ccccc4c23)c1 ; c1ccc(-c2ccc(-c3nc(-c4ccc5ccc6ccccc6c5c4)nc(-c4ccccc4-c4cccc5oc6ccccc6c45)n3)cc2)cc1]

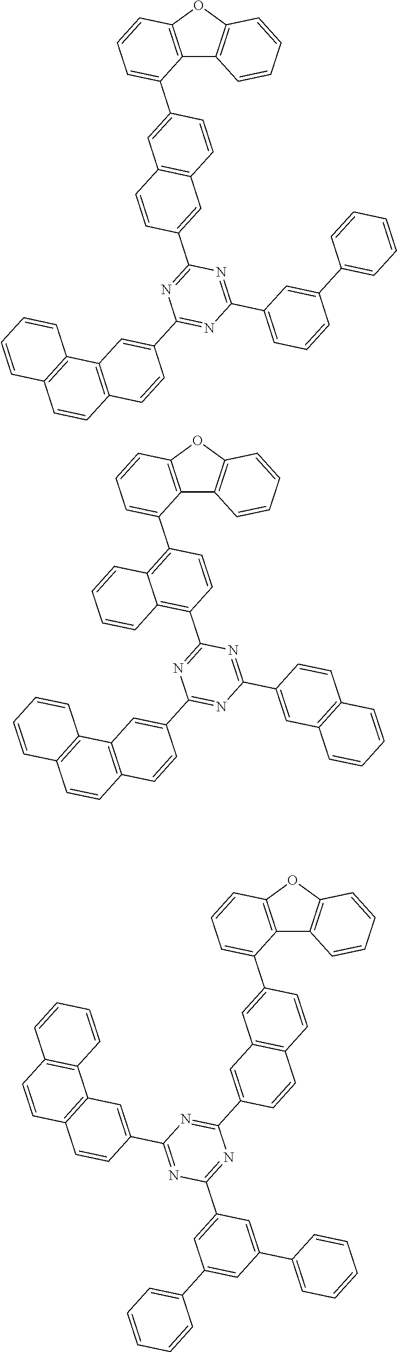 [SMILES: c1ccc(-c2cccc(-c3nc(-c4ccc5cc(-c6cccc7oc8ccccc8c67)ccc5c4)nc(-c4ccc5ccc6ccccc6c5c4)n3)c2)cc1 ; c1ccc2cc(-c3nc(-c4ccc5ccc6ccccc6c5c4)nc(-c4ccc(-c5cccc6oc7ccccc7c56)c5ccccc45)n3)ccc2c1 ; c1ccc(-c2cc(-c3ccccc3)cc(-c3nc(-c4ccc5ccc(-c6cccc7oc8ccccc8c67)cc5c4)nc(-c4ccc5ccc6ccccc6c5c4)n3)c2)cc1]

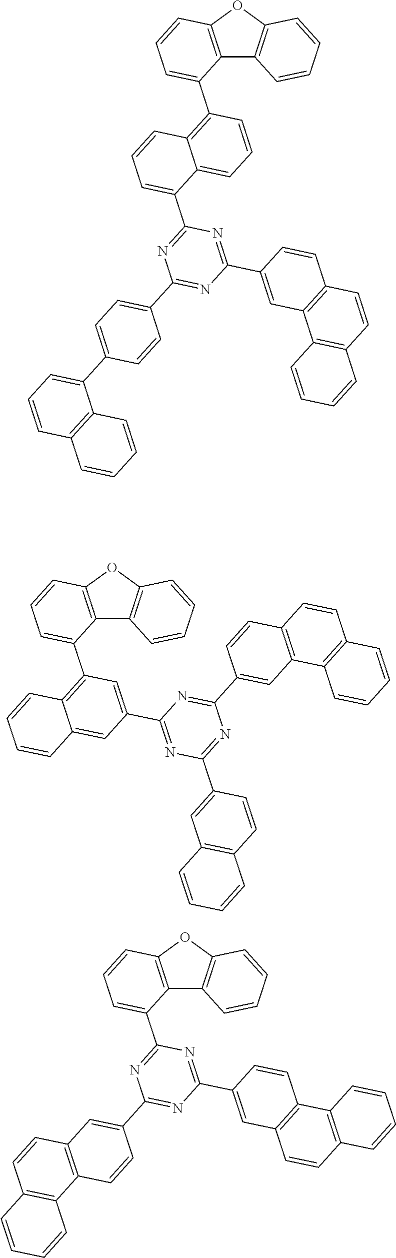

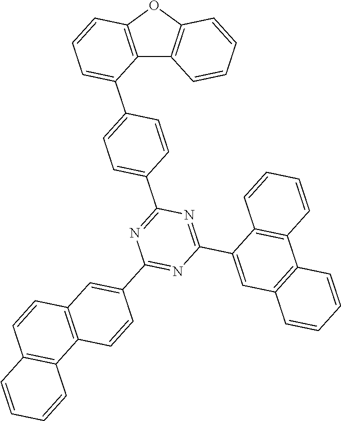 [SMILES: c1ccc2c(c1)ccc1cc(-c3nc(-c4ccc(-c5cccc6oc7ccccc7c56)cc4)nc(-c4cc5ccccc5c5ccccc45)n3)ccc12]

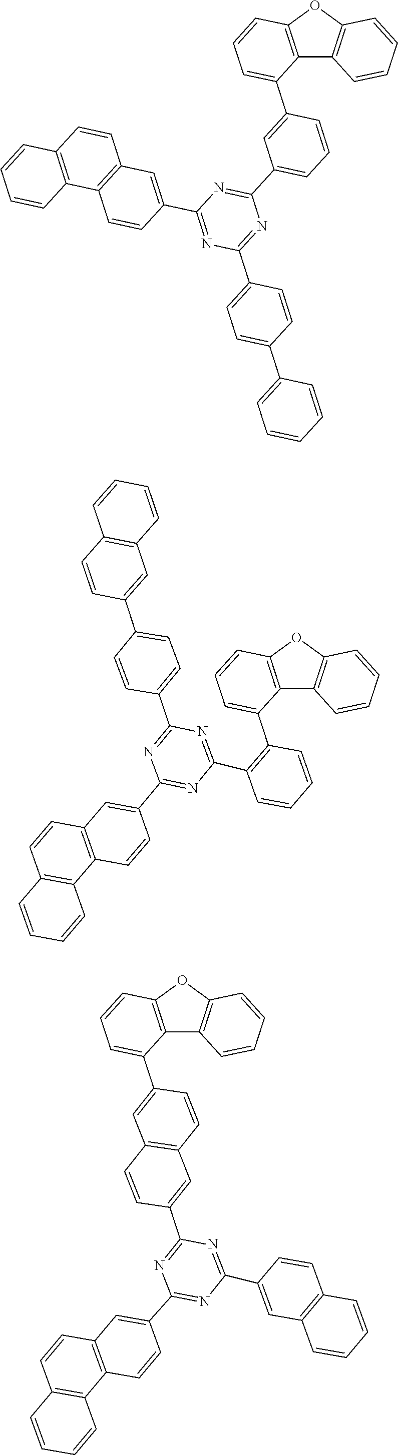 [SMILES: c1ccc(-c2ccc(-c3nc(-c4cccc(-c5cccc6oc7ccccc7c56)c4)nc(-c4ccc5c(ccc6ccccc65)c4)n3)cc2)cc1 ; c1ccc(-c2cccc3oc4ccccc4c23)c(-c2nc(-c3ccc(-c4ccc5ccccc5c4)cc3)nc(-c3ccc4c(ccc5ccccc54)c3)n2)c1 ; c1ccc2cc(-c3nc(-c4ccc5cc(-c6cccc7oc8ccccc8c67)ccc5c4)nc(-c4ccc5c(ccc6ccccc65)c4)n3)ccc2c1]

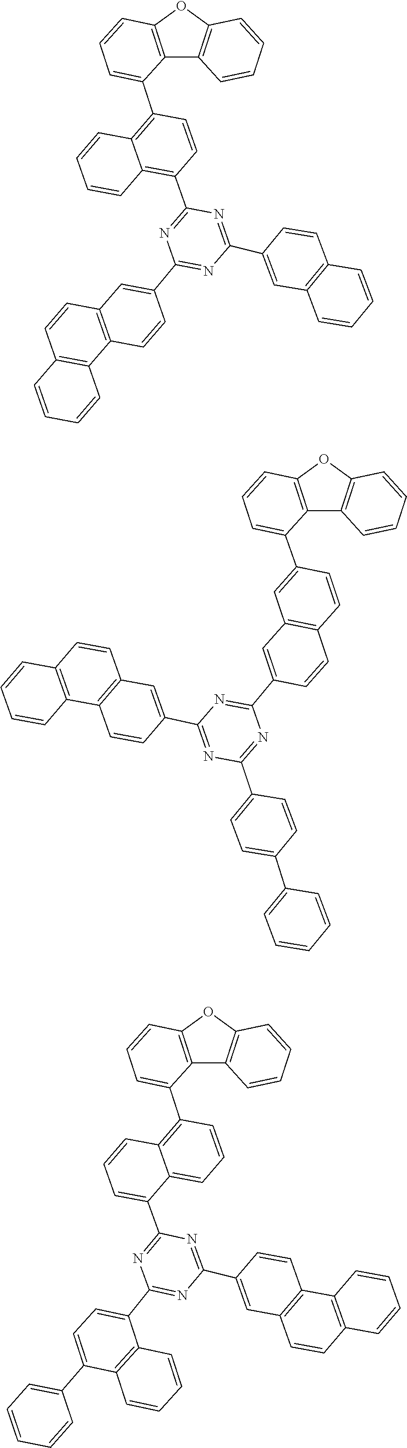 [SMILES: c1ccc2cc(-c3nc(-c4ccc5c(ccc6ccccc65)c4)nc(-c4ccc(-c5cccc6oc7ccccc7c56)c5ccccc45)n3)ccc2c1 ; c1ccc(-c2ccc(-c3nc(-c4ccc5ccc(-c6cccc7oc8ccccc8c67)cc5c4)nc(-c4ccc5c(ccc6ccccc65)c4)n3)cc2)cc1 ; c1ccc(-c2ccc(-c3nc(-c4ccc5c(ccc6ccccc65)c4)nc(-c4cccc5c(-c6cccc7oc8ccccc8c67)cccc45)n3)c3ccccc23)cc1]

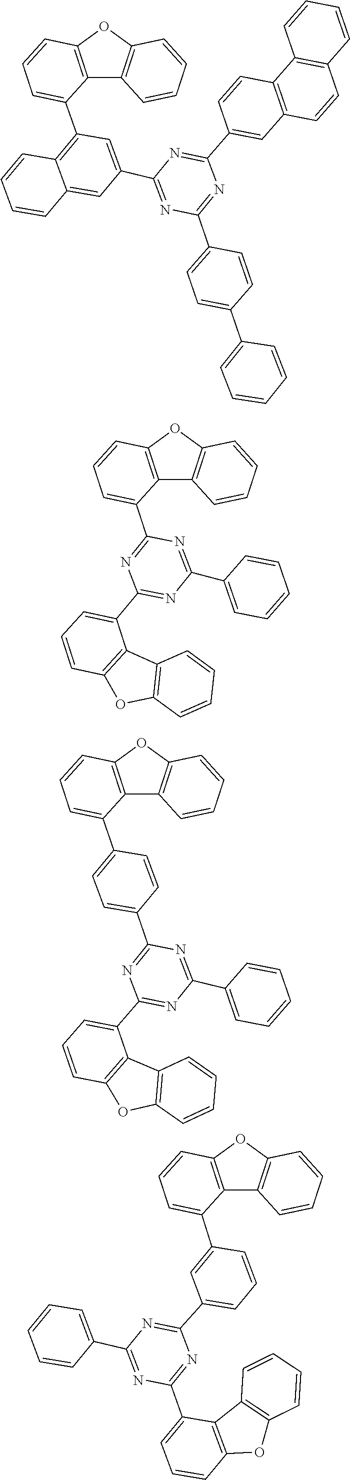

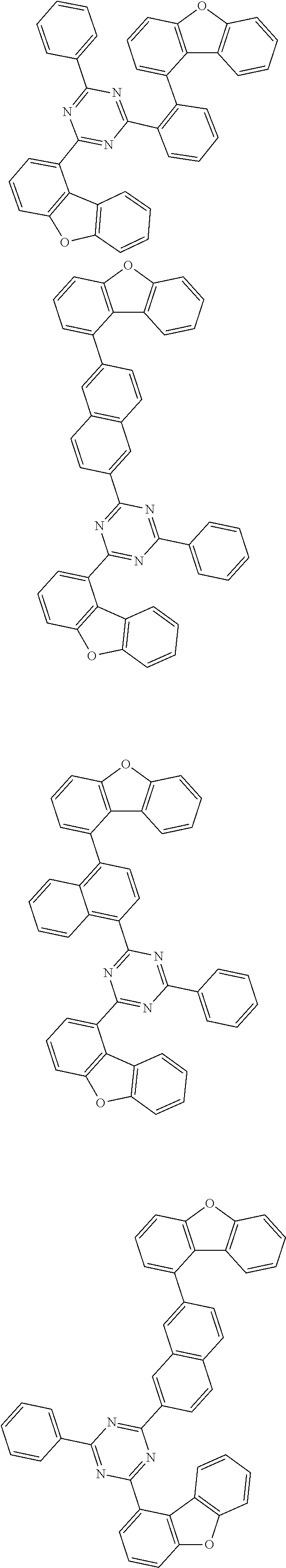

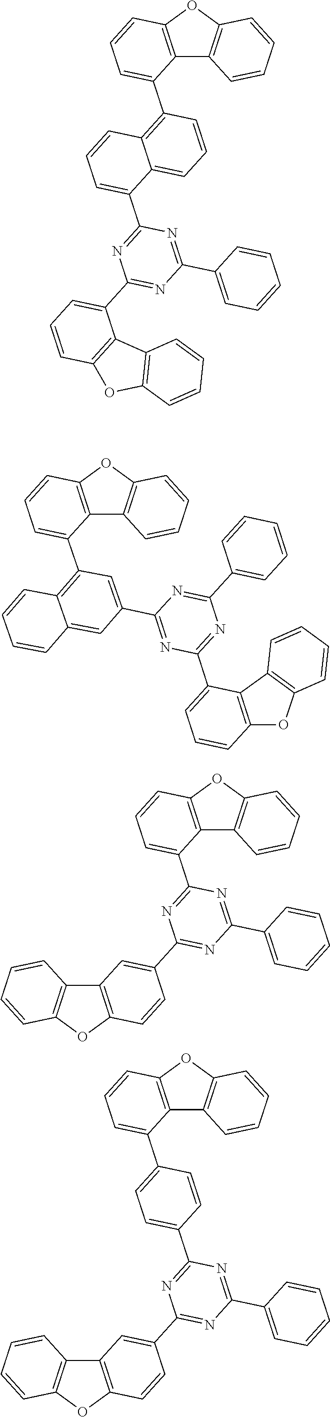

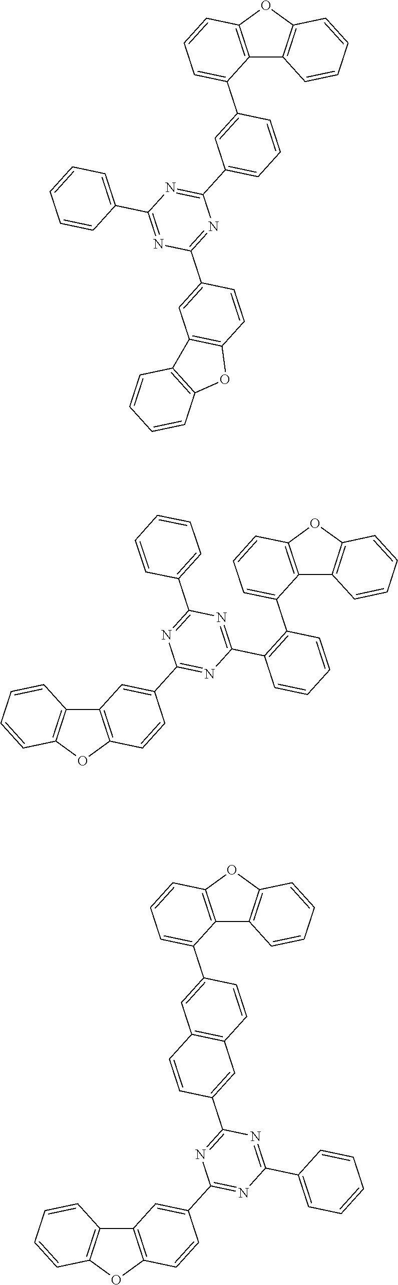 [SMILES: c1ccc(-c2nc(-c3cccc(-c4cccc5oc6ccccc6c45)c3)nc(-c3ccc4oc5ccccc5c4c3)n2)cc1 ; c1ccc(-c2nc(-c3ccc4oc5ccccc5c4c3)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4oc5ccccc5c4c3)n2)cc1]

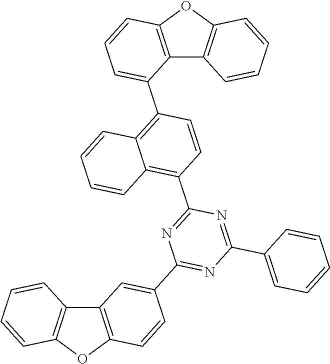 [SMILES: c1ccc(-c2nc(-c3ccc4oc5ccccc5c4c3)nc(-c3ccc(-c4cccc5oc6ccccc6c45)c4ccccc34)n2)cc1]

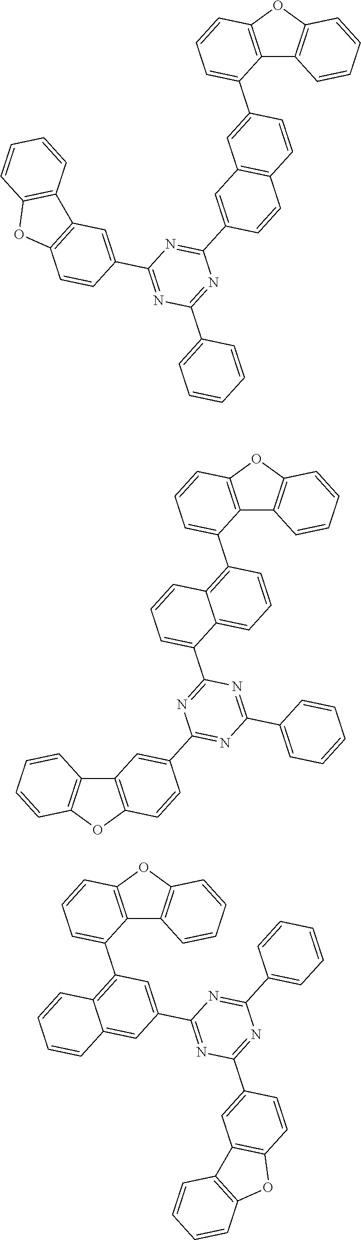 [SMILES: c1ccc(-c2nc(-c3ccc4ccc(-c5cccc6oc7ccccc7c56)cc4c3)nc(-c3ccc4oc5ccccc5c4c3)n2)cc1 ; c1ccc(-c2nc(-c3ccc4oc5ccccc5c4c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4oc5ccccc5c4c3)n2)cc1]

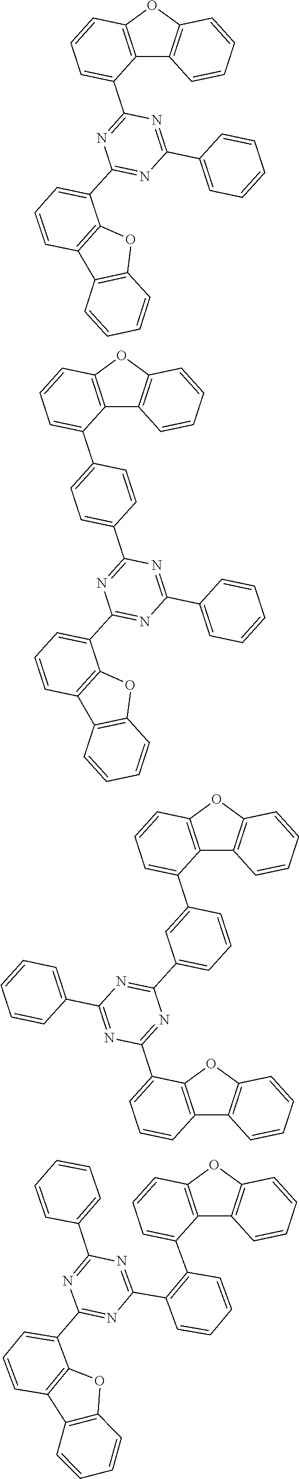

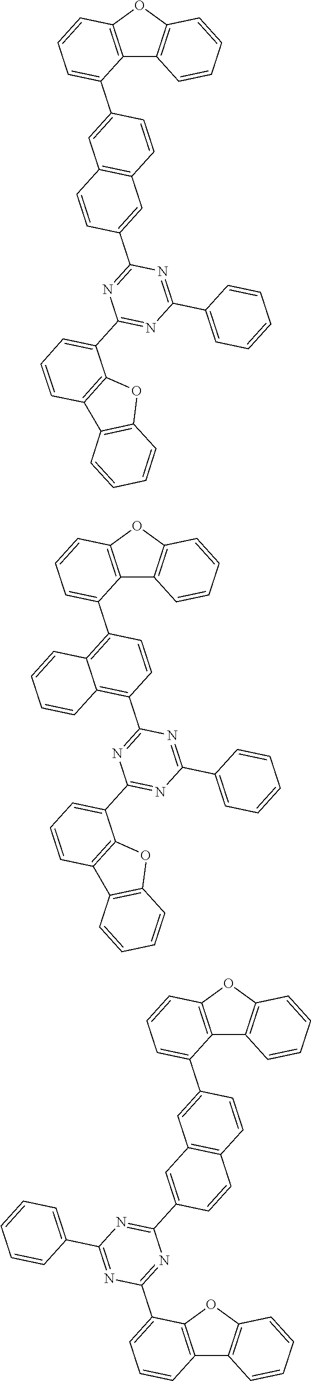

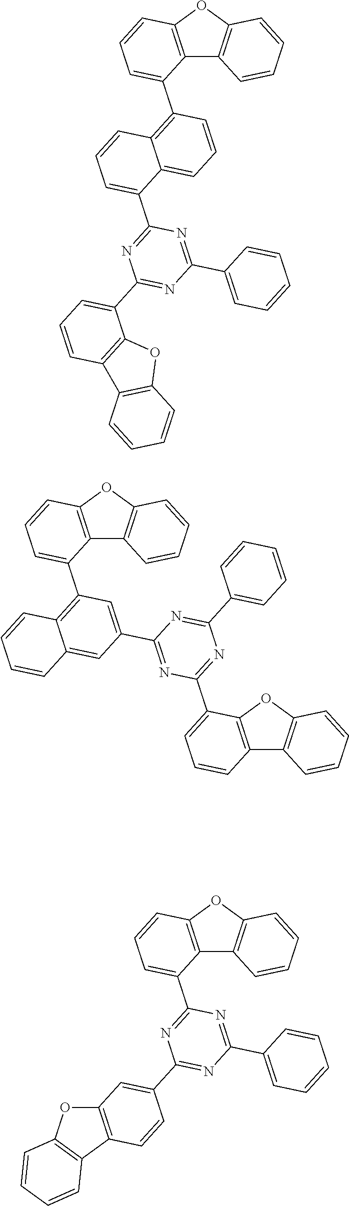

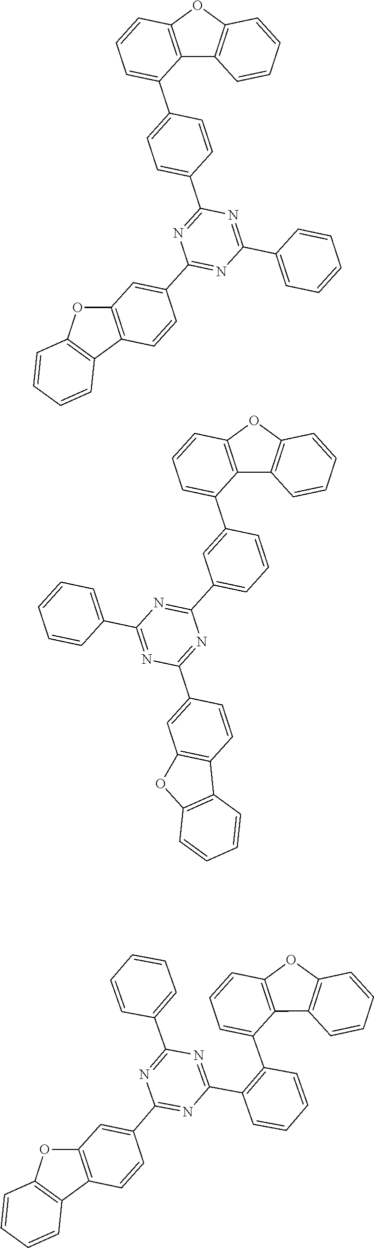 [SMILES: c1ccc(-c2nc(-c3ccc(-c4cccc5oc6ccccc6c45)cc3)nc(-c3ccc4c(c3)oc3ccccc34)n2)cc1 ; c1ccc(-c2nc(-c3cccc(-c4cccc5oc6ccccc6c45)c3)nc(-c3ccc4c(c3)oc3ccccc34)n2)cc1 ; c1ccc(-c2nc(-c3ccc4c(c3)oc3ccccc34)nc(-c3ccccc3-c3cccc4oc5ccccc5c34)n2)cc1]

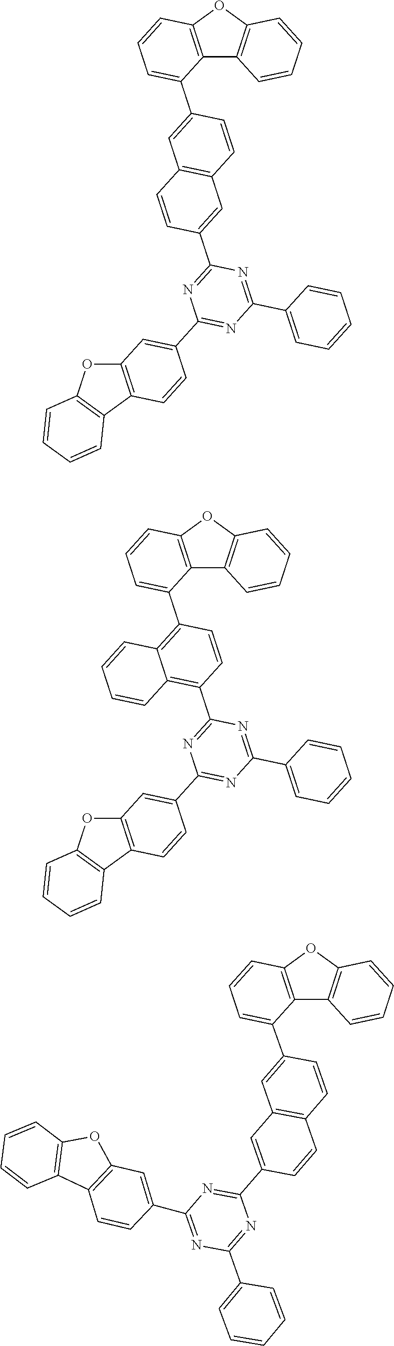 [SMILES: c1ccc(-c2nc(-c3ccc4cc(-c5cccc6oc7ccccc7c56)ccc4c3)nc(-c3ccc4c(c3)oc3ccccc34)n2)cc1 ; c1ccc(-c2nc(-c3ccc4c(c3)oc3ccccc34)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(c3)oc3ccccc34)n2)cc1]

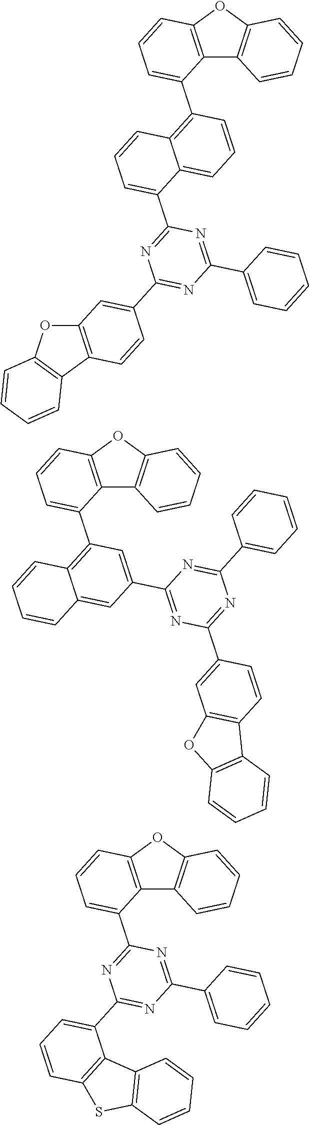

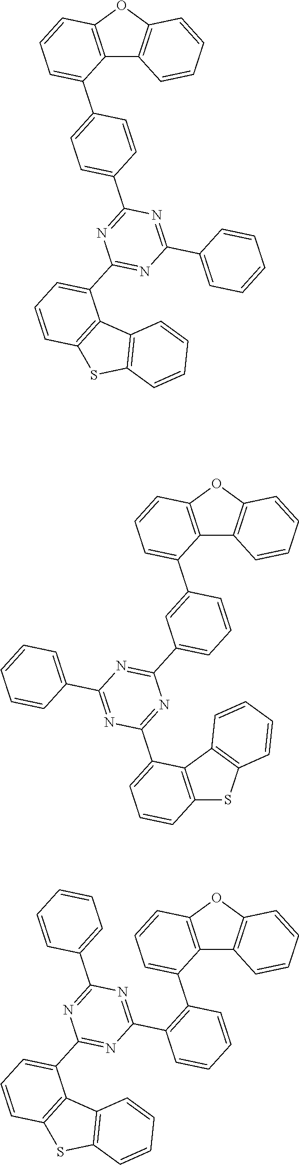 [SMILES: c1ccc(-c2nc(-c3ccc(-c4cccc5oc6ccccc6c45)cc3)nc(-c3cccc4sc5ccccc5c34)n2)cc1 ; c1ccc(-c2nc(-c3cccc(-c4cccc5oc6ccccc6c45)c3)nc(-c3cccc4sc5ccccc5c34)n2)cc1 ; c1ccc(-c2nc(-c3ccccc3-c3cccc4oc5ccccc5c34)nc(-c3cccc4sc5ccccc5c34)n2)cc1]

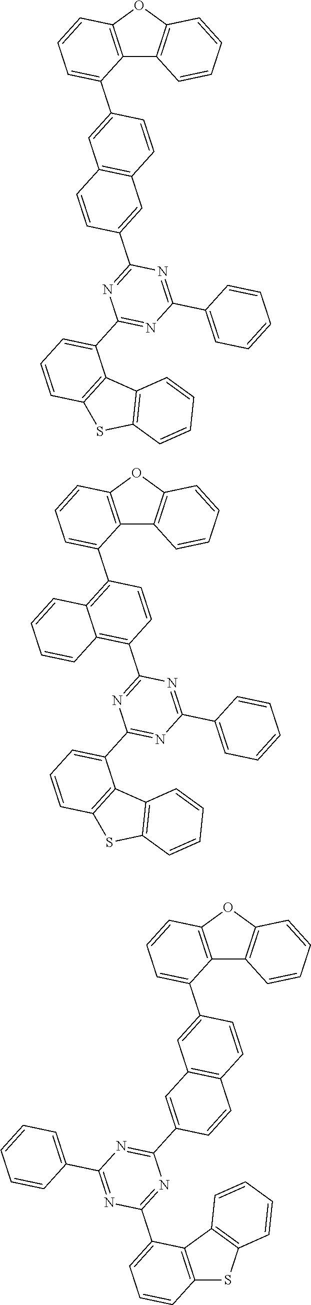

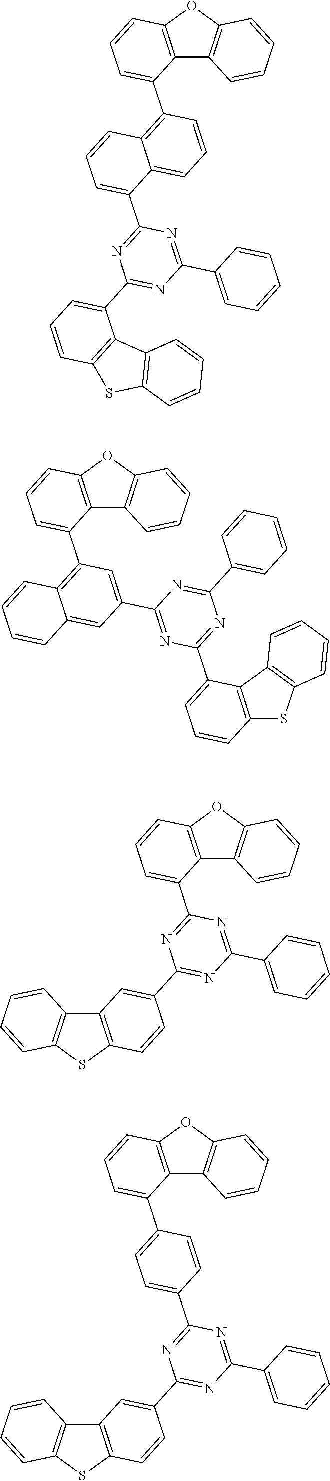 [SMILES: c1ccc(-c2nc(-c3cccc4c(-c5cccc6oc7ccccc7c56)cccc34)nc(-c3cccc4sc5ccccc5c34)n2)cc1 ; c1ccc(-c2nc(-c3cc(-c4cccc5oc6ccccc6c45)c4ccccc4c3)nc(-c3cccc4sc5ccccc5c34)n2)cc1 ; c1ccc(-c2nc(-c3ccc4sc5ccccc5c4c3)nc(-c3cccc4oc5ccccc5c34)n2)cc1 ; c1ccc(-c2nc(-c3ccc(-c4cccc5oc6ccccc6c45)cc3)nc(-c3ccc4sc5ccccc5c4c3)n2)cc1]

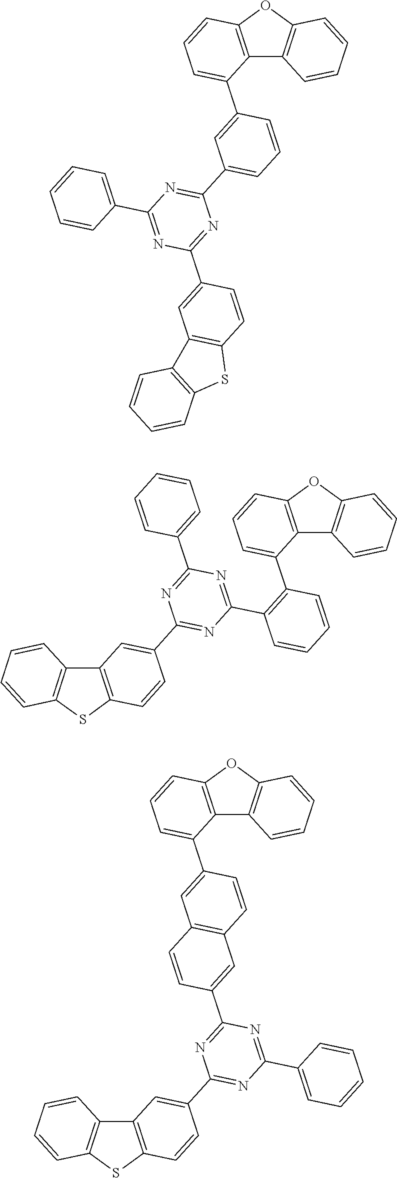

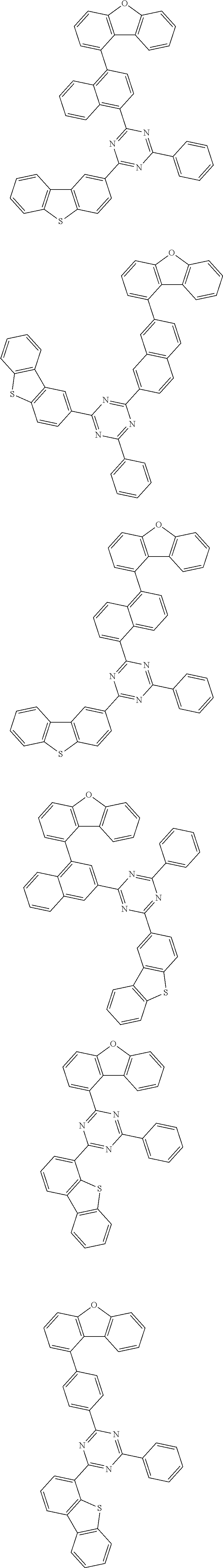

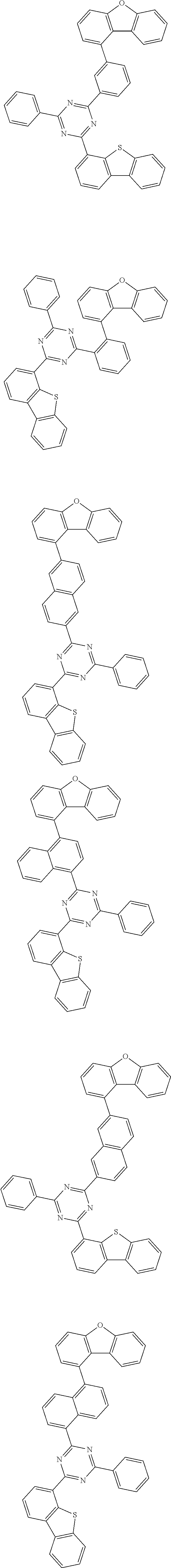

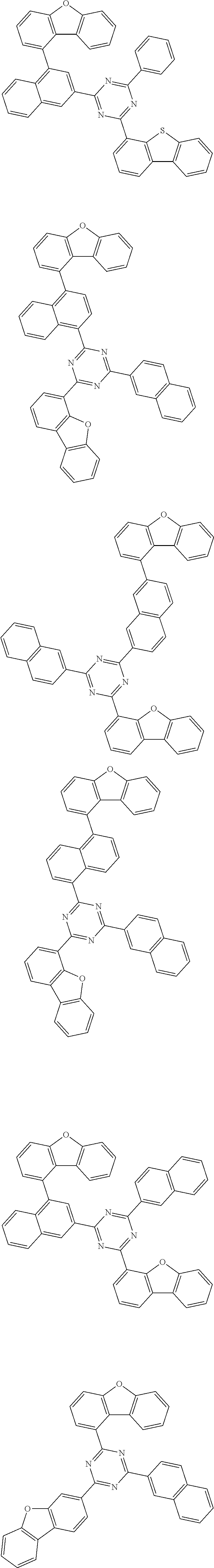

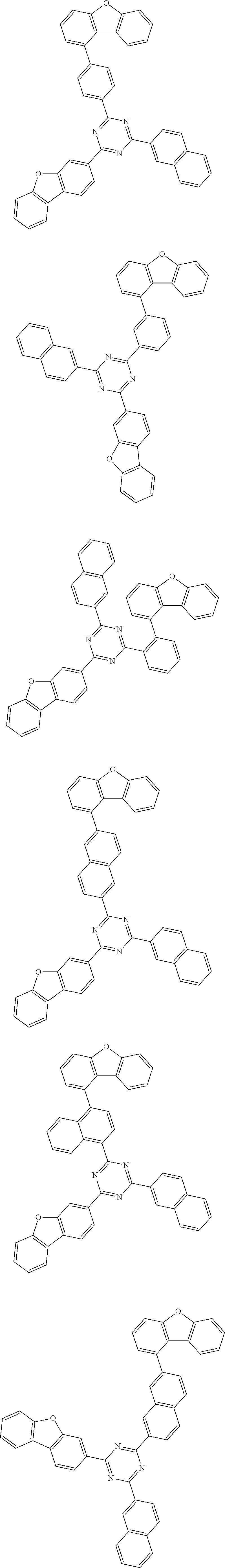

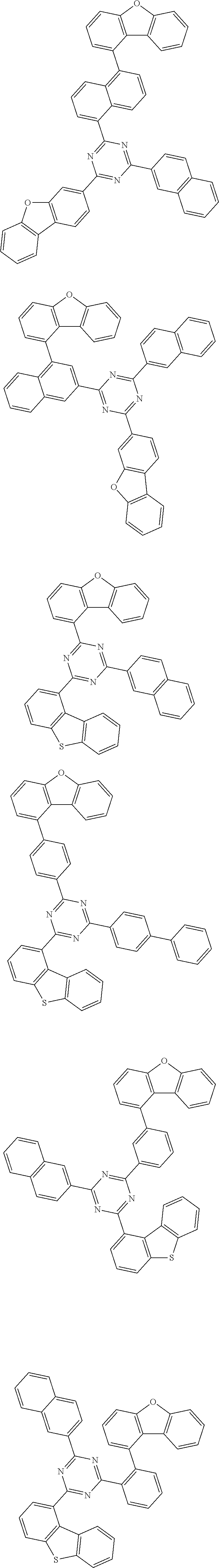

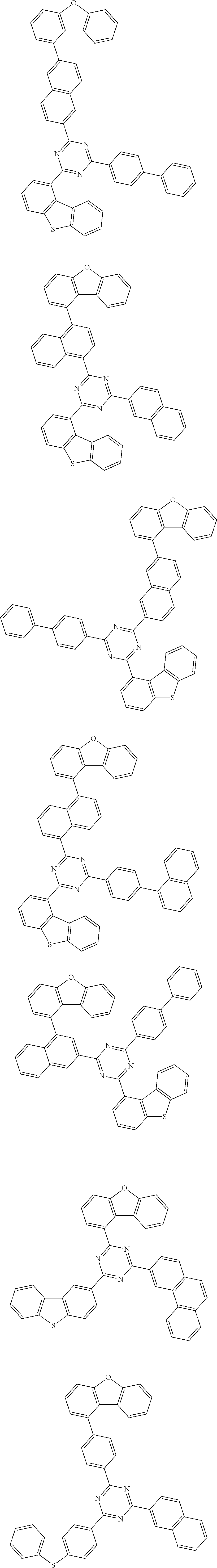

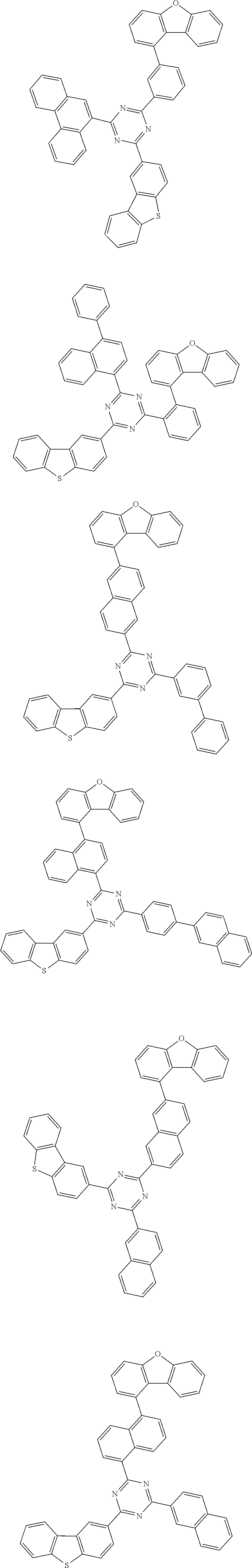

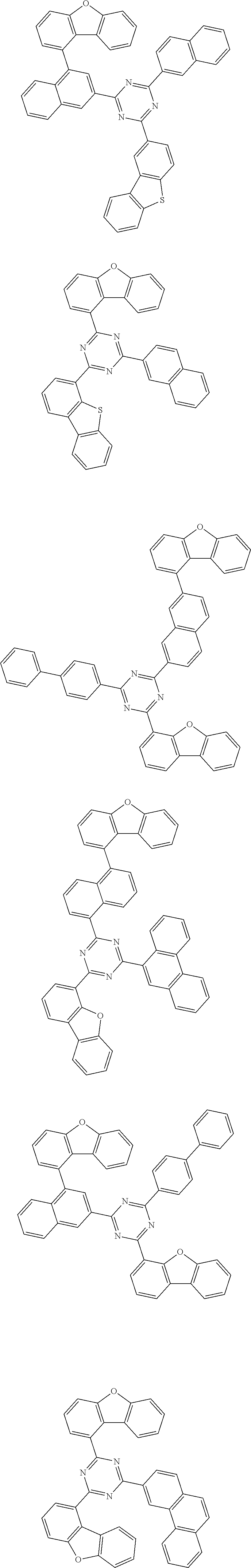

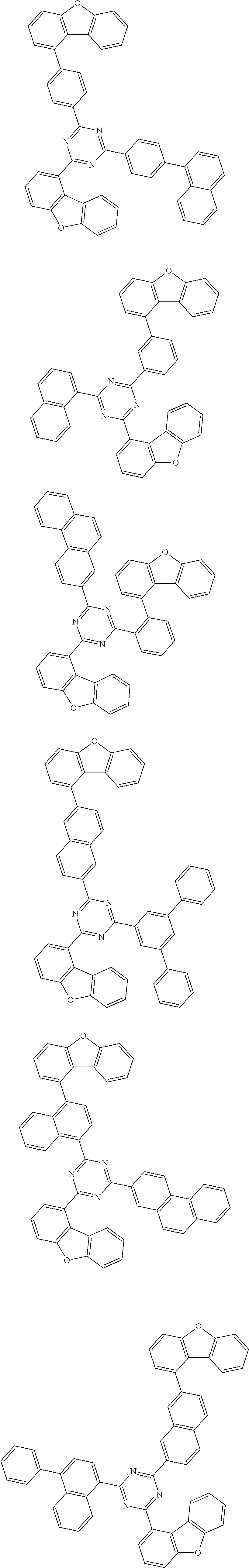

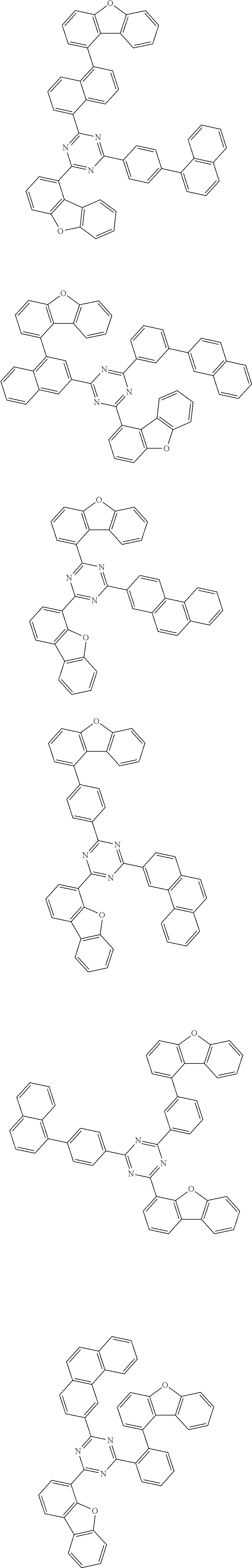

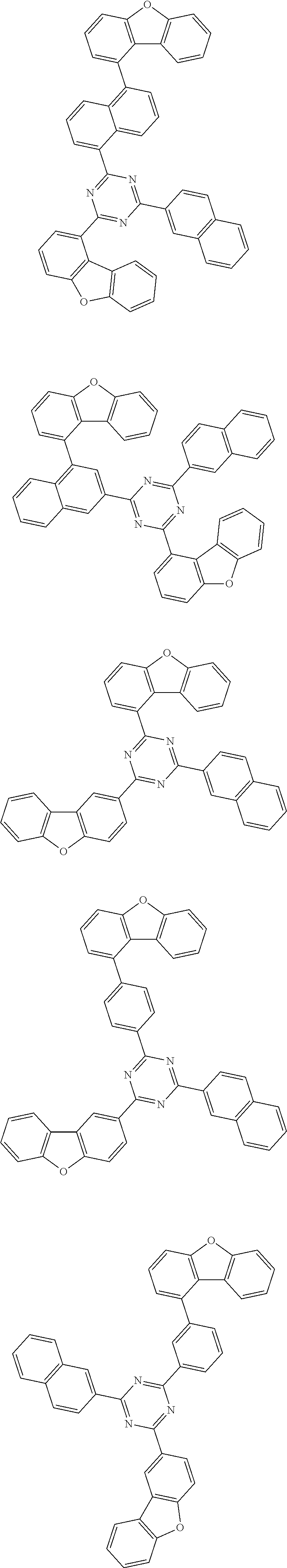 [SMILES: c1ccc2cc(-c3nc(-c4cccc5c(-c6cccc7oc8ccccc8c67)cccc45)nc(-c4cccc5oc6ccccc6c45)n3)ccc2c1 ; c1ccc2cc(-c3nc(-c4cc(-c5cccc6oc7ccccc7c56)c5ccccc5c4)nc(-c4cccc5oc6ccccc6c45)n3)ccc2c1 ; c1ccc2cc(-c3nc(-c4ccc5oc6ccccc6c5c4)nc(-c4cccc5oc6ccccc6c45)n3)ccc2c1 ; c1ccc2cc(-c3nc(-c4ccc(-c5cccc6oc7ccccc7c56)cc4)nc(-c4ccc5oc6ccccc6c5c4)n3)ccc2c1 ; c1cc(-c2nc(-c3ccc4ccccc4c3)nc(-c3ccc4oc5ccccc5c4c3)n2)cc(-c2cccc3oc4ccccc4c23)c1]

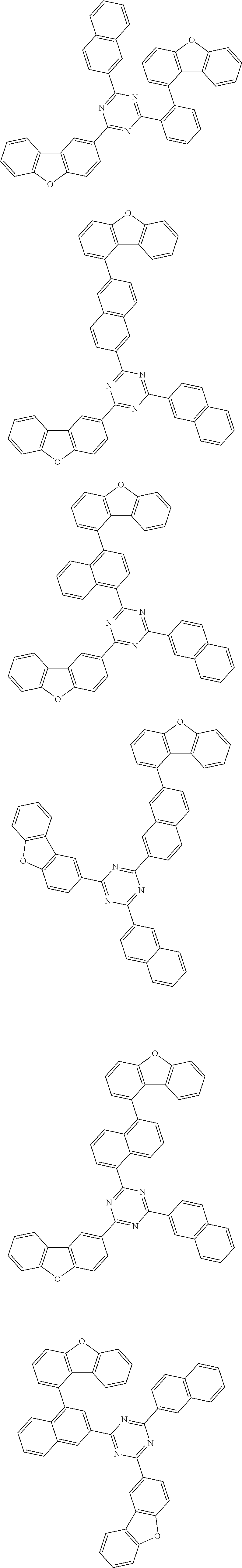

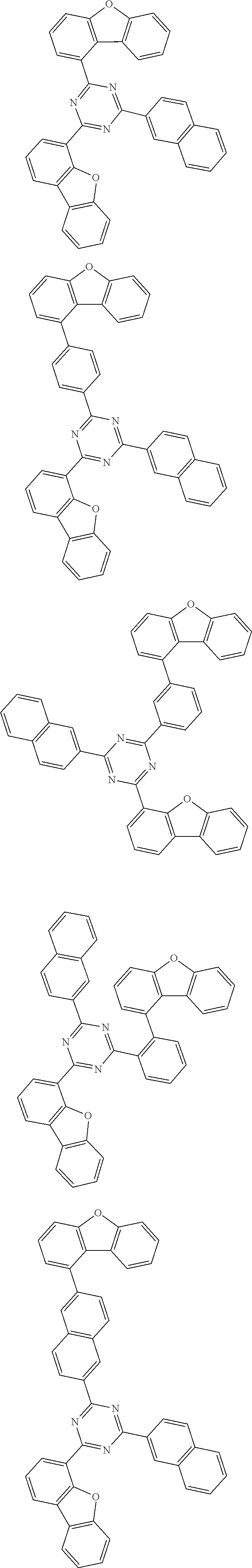

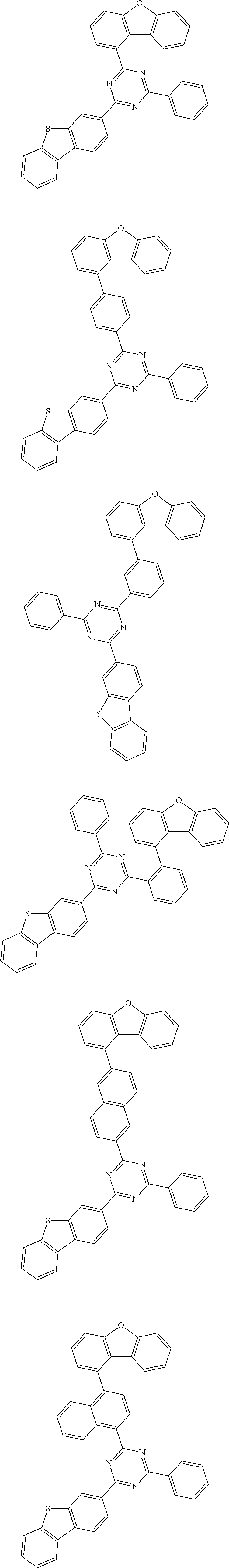 [SMILES: c1ccc(-c2nc(-c3ccc4c(c3)sc3ccccc34)nc(-c3cccc4oc5ccccc5c34)n2)cc1 ; c1ccc(-c2nc(-c3ccc(-c4cccc5oc6ccccc6c45)cc3)nc(-c3ccc4c(c3)sc3ccccc34)n2)cc1 ; c1ccc(-c2nc(-c3cccc(-c4cccc5oc6ccccc6c45)c3)nc(-c3ccc4c(c3)sc3ccccc34)n2)cc1 ; c1ccc(-c2nc(-c3ccc4c(c3)sc3ccccc34)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(c3)sc3ccccc34)n2)cc1 ; c1ccc(-c2nc(-c3ccc4c(c3)sc3ccccc34)nc(-c3ccc(-c4cccc5oc6ccccc6c45)c4ccccc34)n2)cc1]

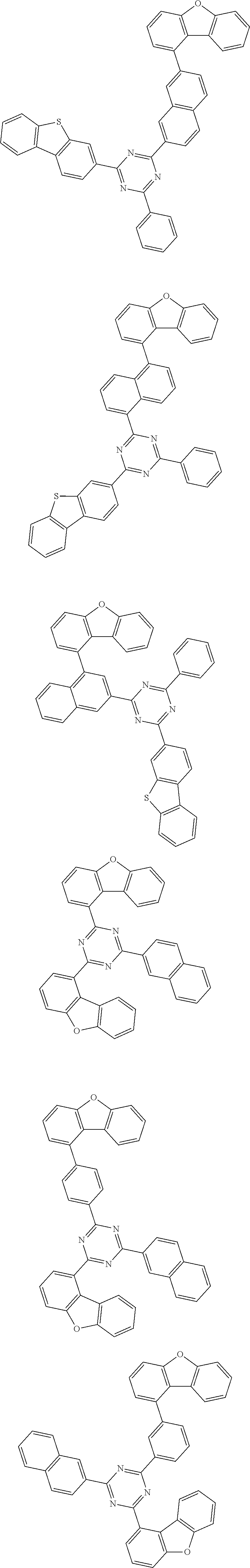 [SMILES: c1ccc(-c2nc(-c3ccc4ccc(-c5cccc6oc7ccccc7c56)cc4c3)nc(-c3ccc4c(c3)sc3ccccc34)n2)cc1 ; c1ccc(-c2nc(-c3ccc4c(c3)sc3ccccc34)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4c(c3)sc3ccccc34)n2)cc1 ; c1ccc2cc(-c3nc(-c4cccc5oc6ccccc6c45)nc(-c4cccc5oc6ccccc6c45)n3)ccc2c1 ; c1ccc2cc(-c3nc(-c4ccc(-c5cccc6oc7ccccc7c56)cc4)nc(-c4cccc5oc6ccccc6c45)n3)ccc2c1 ; c1cc(-c2nc(-c3ccc4ccccc4c3)nc(-c3cccc4oc5ccccc5c34)n2)cc(-c2cccc3oc4ccccc4c23)c1]

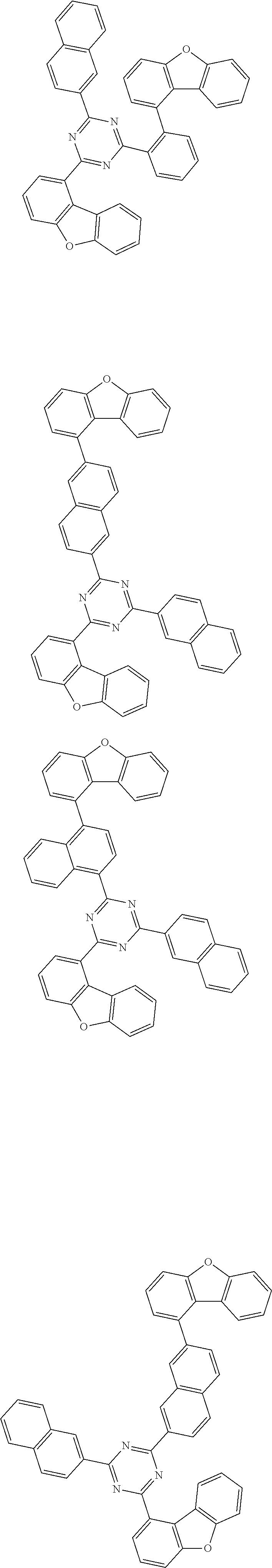

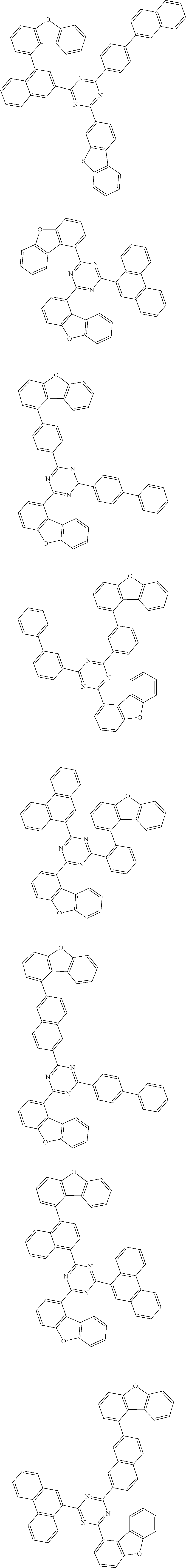

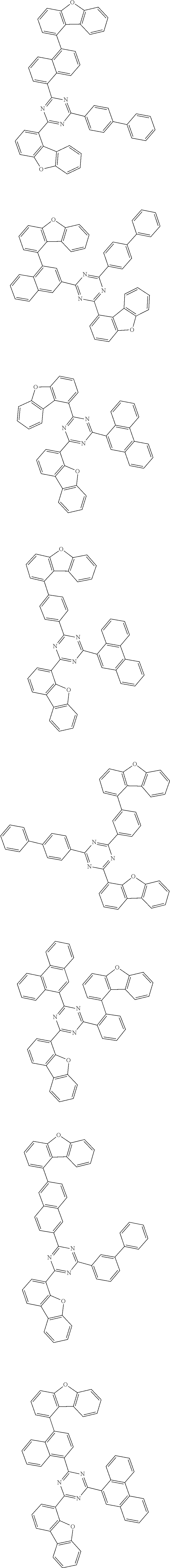

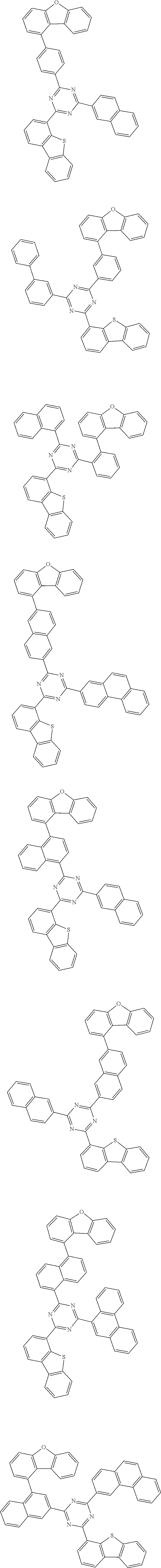

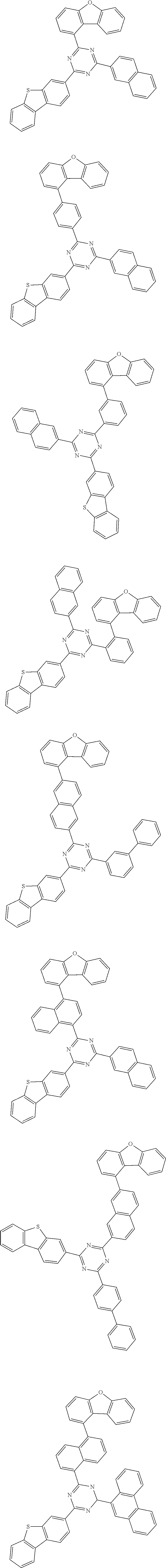

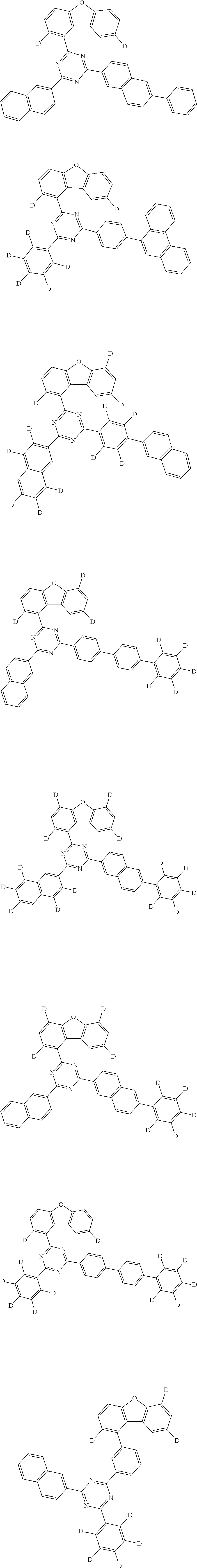

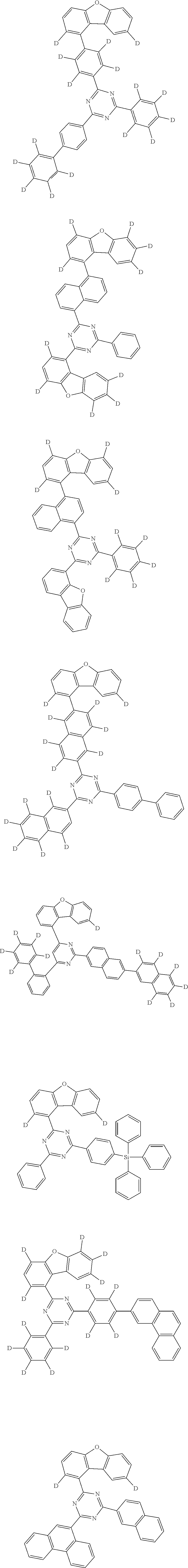

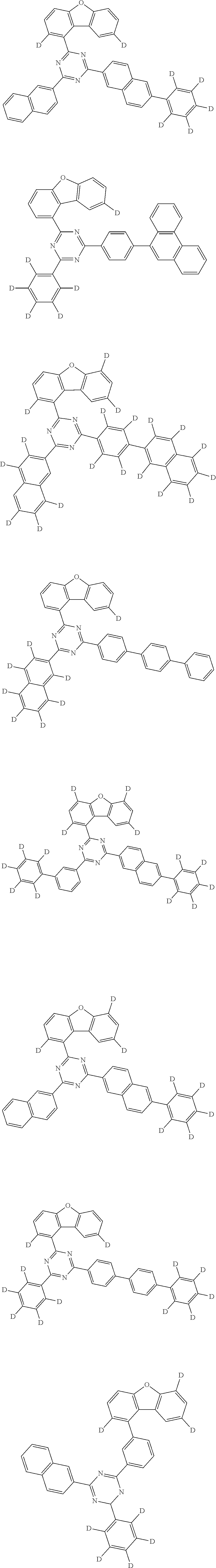

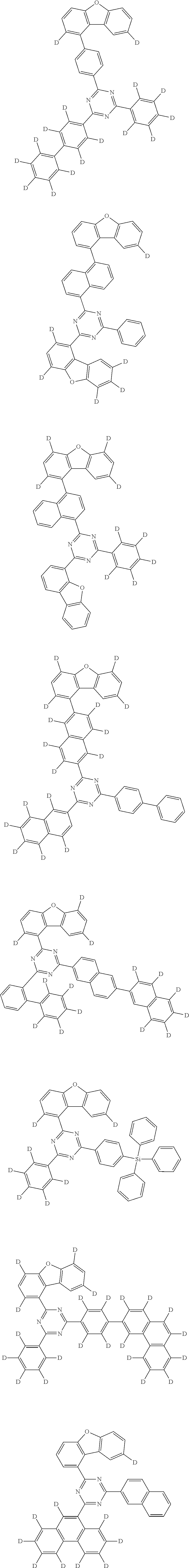

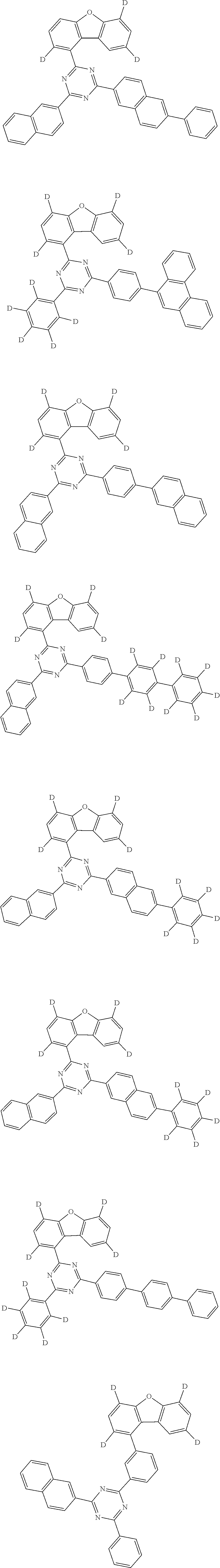

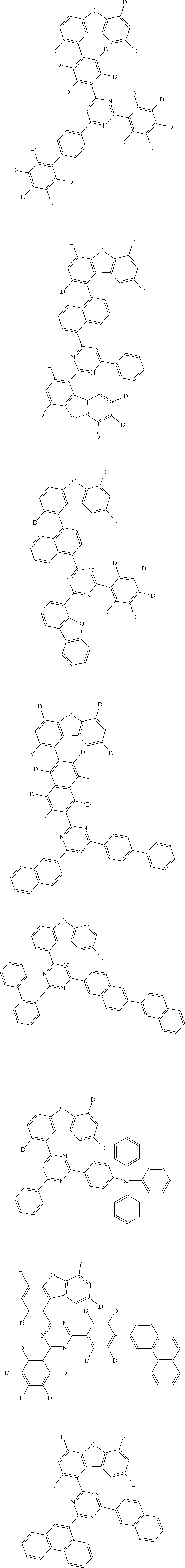

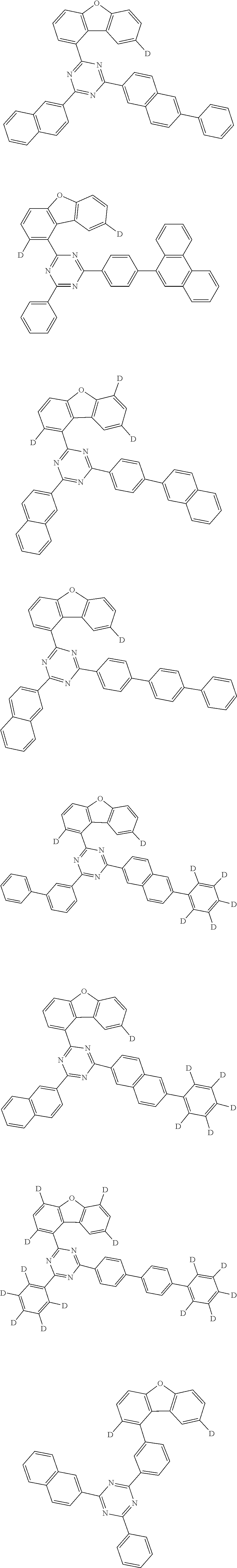

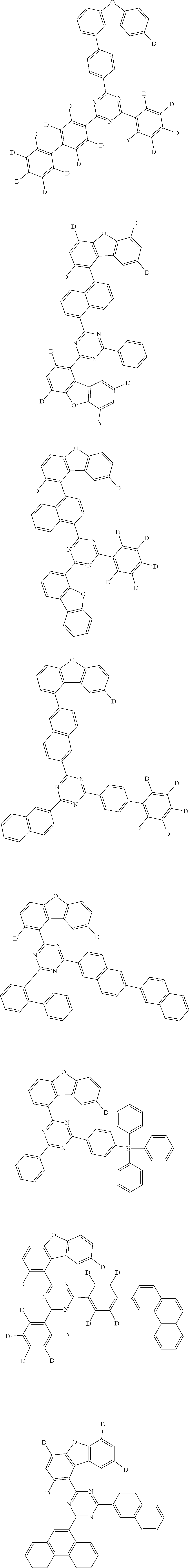

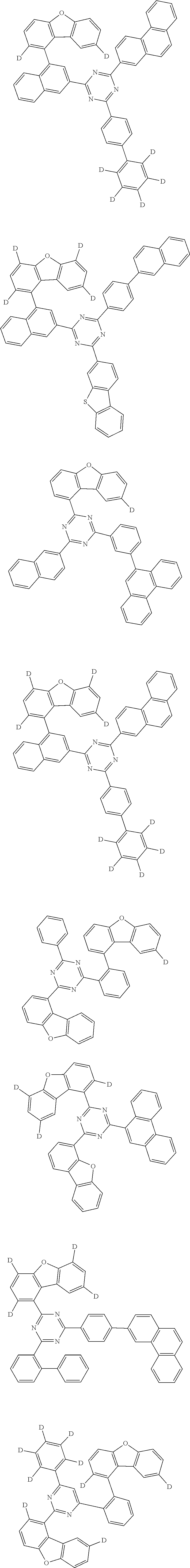

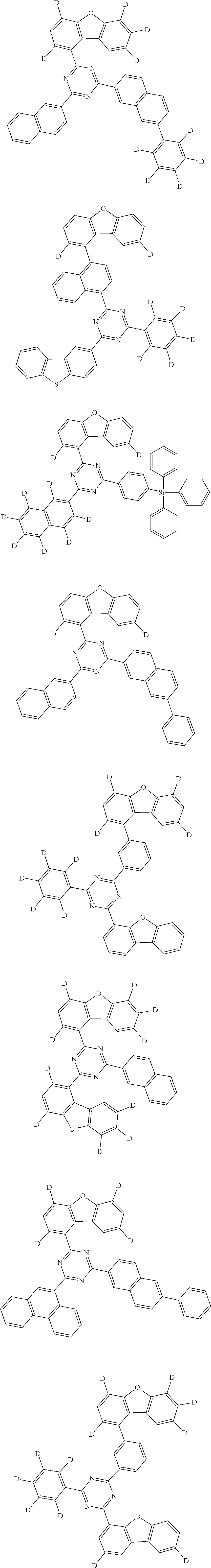

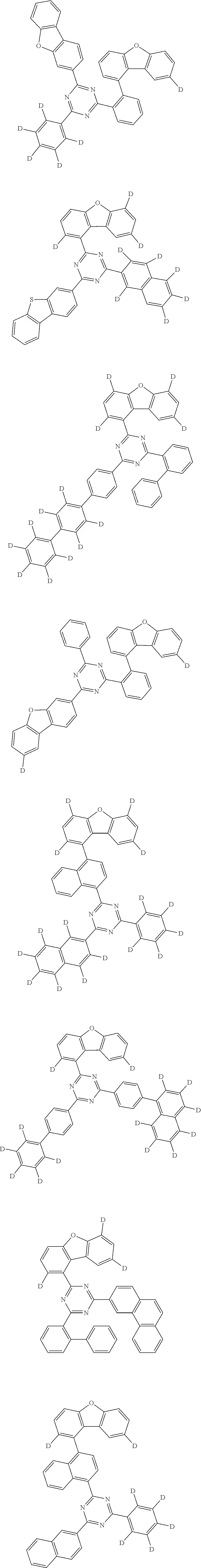

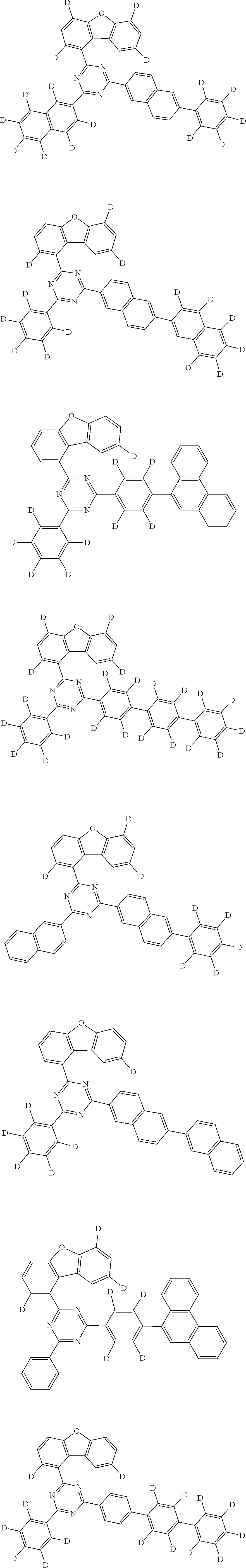

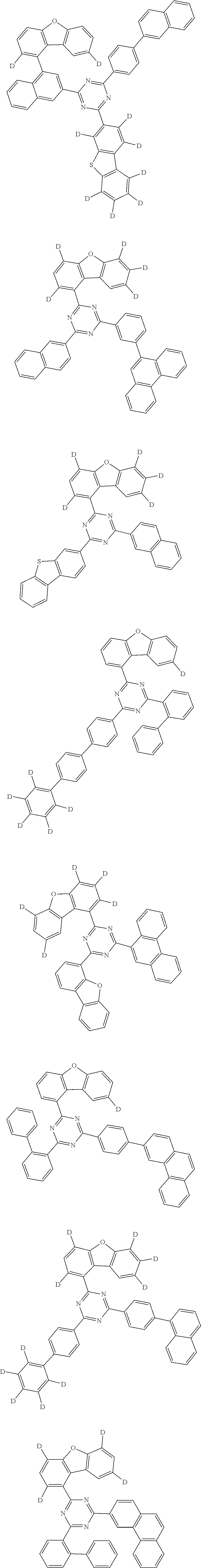

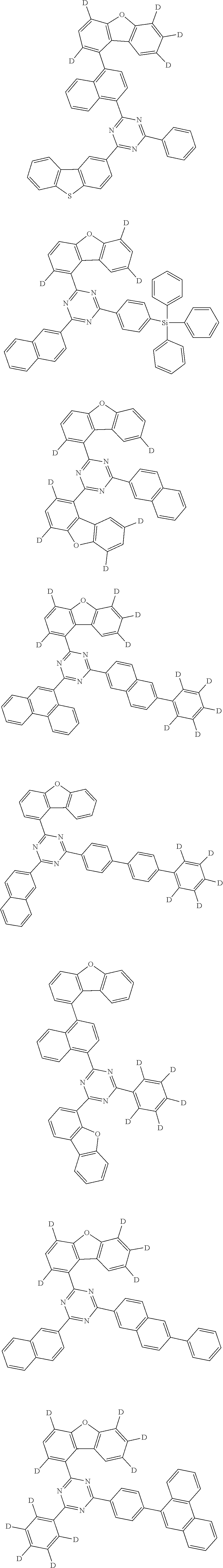

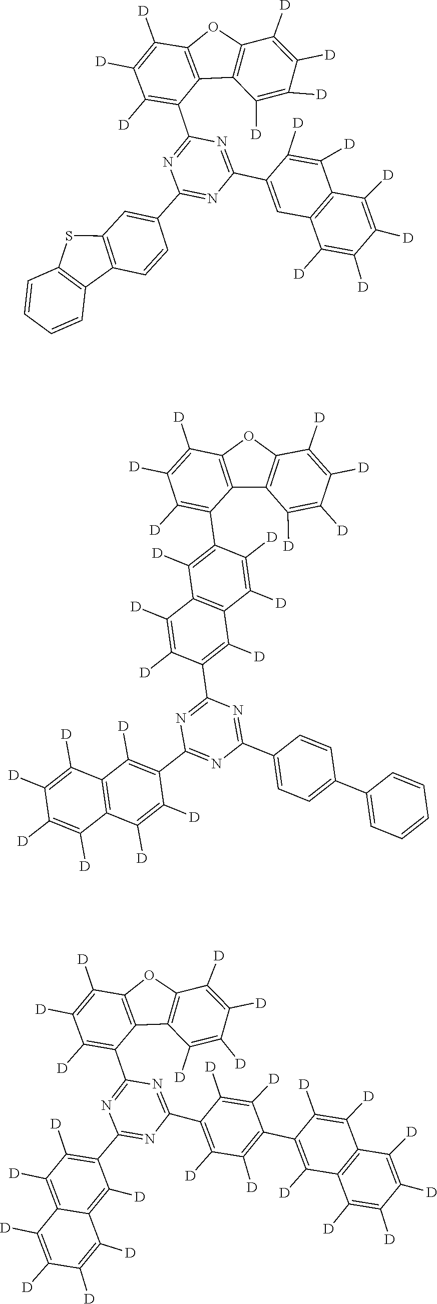

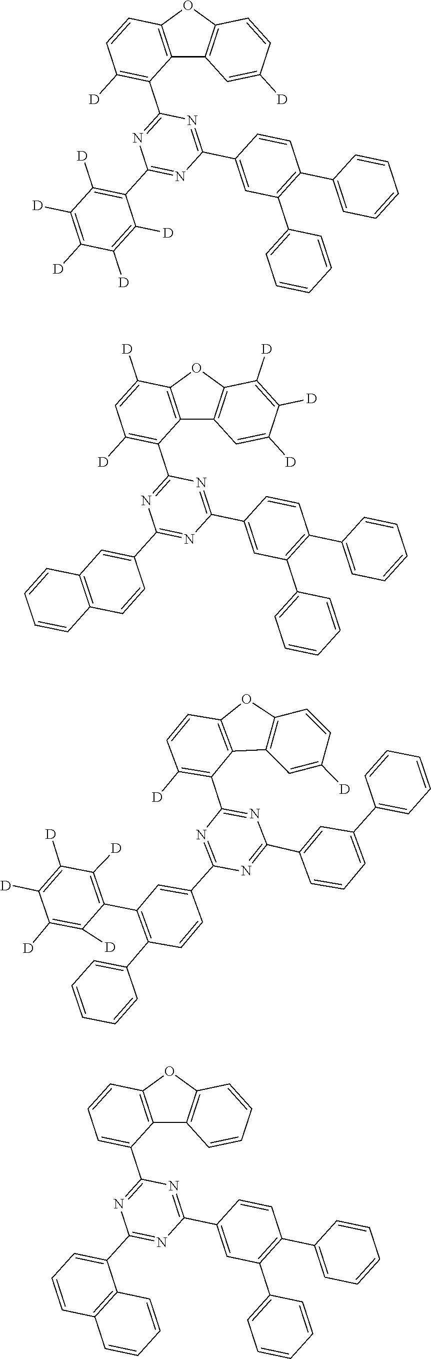 [SMILES: [2H]c1ccc2oc3ccc([2H])c(-c4nc(-c5ccc(-c6ccccc6)c(-c6ccccc6)c5)nc(-c5c([2H])c([2H])c([2H])c([2H])c5[2H])n4)c3c2c1 ; [2H]c1cc2c(oc3c([2H])cc([2H])c(-c4nc(-c5ccc(-c6ccccc6)c(-c6ccccc6)c5)nc(-c5ccc6ccccc6c5)n4)c32)c([2H])c1[2H] ; [2H]c1ccc2oc3ccc([2H])c(-c4nc(-c5cccc(-c6ccccc6)c5)nc(-c5ccc(-c6ccccc6)c(-c6c([2H])c([2H])c([2H])c([2H])c6[2H])c5)n4)c3c2c1 ; c1ccc(-c2ccc(-c3nc(-c4cccc5ccccc45)nc(-c4cccc5oc6ccccc6c45)n3)cc2-c2ccccc2)cc1]

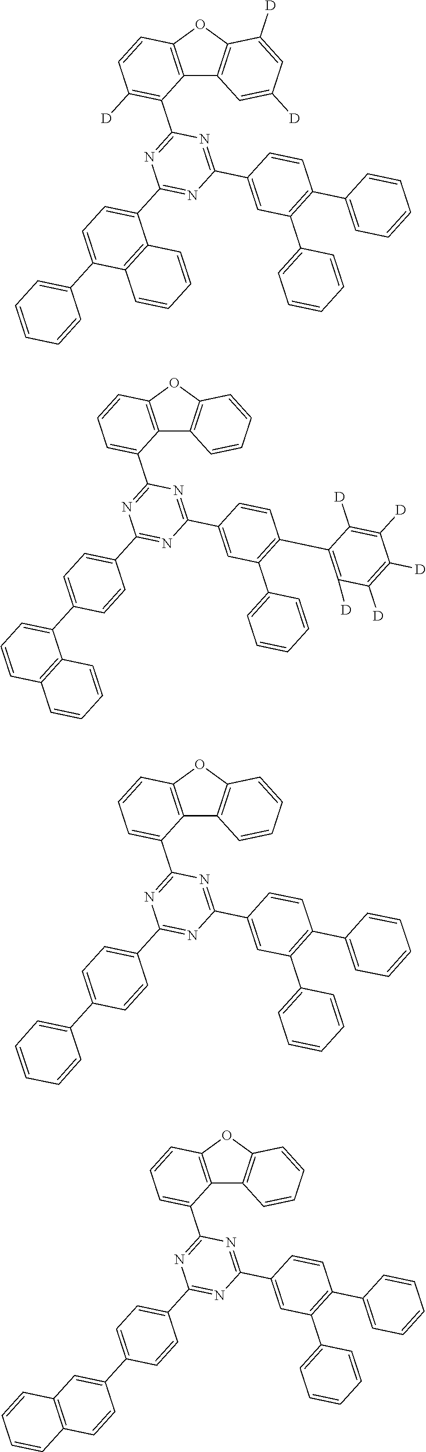 [SMILES: [2H]c1cc([2H])c2oc3ccc([2H])c(-c4nc(-c5ccc(-c6ccccc6)c(-c6ccccc6)c5)nc(-c5ccc(-c6ccccc6)c6ccccc56)n4)c3c2c1 ; [2H]c1c([2H])c([2H])c(-c2ccc(-c3nc(-c4ccc(-c5cccc6ccccc56)cc4)nc(-c4cccc5oc6ccccc6c45)n3)cc2-c2ccccc2)c([2H])c1[2H] ; c1ccc(-c2ccc(-c3nc(-c4ccc(-c5ccccc5)c(-c5ccccc5)c4)nc(-c4cccc5oc6ccccc6c45)n3)cc2)cc1 ; c1ccc(-c2ccc(-c3nc(-c4ccc(-c5ccc6ccccc6c5)cc4)nc(-c4cccc5oc6ccccc6c45)n3)cc2-c2ccccc2)cc1]

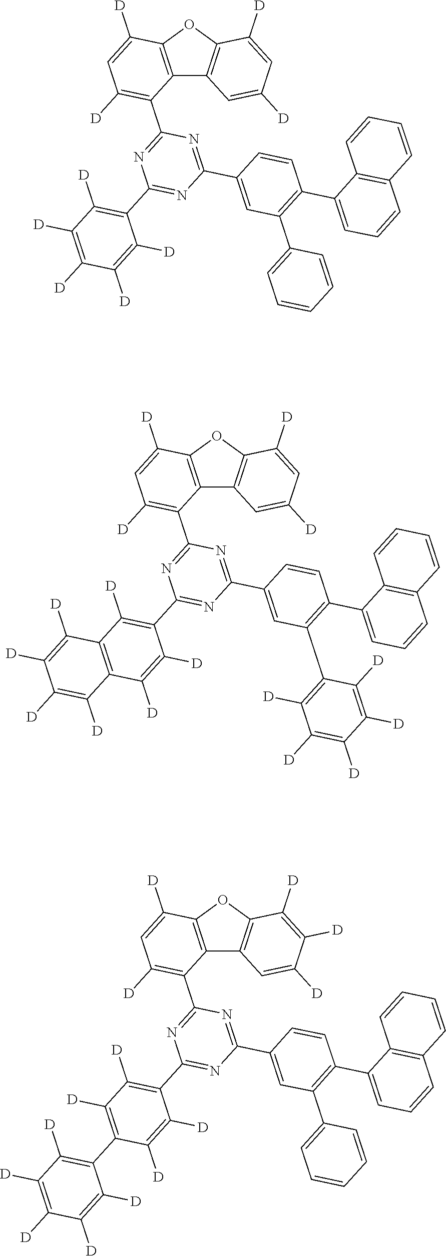 [SMILES: [2H]c1cc([2H])c2oc3c([2H])cc([2H])c(-c4nc(-c5ccc(-c6cccc7ccccc67)c(-c6ccccc6)c5)nc(-c5c([2H])c([2H])c([2H])c([2H])c5[2H])n4)c3c2c1 ; [2H]c1cc([2H])c2oc3c([2H])cc([2H])c(-c4nc(-c5ccc(-c6cccc7ccccc67)c(-c6c([2H])c([2H])c([2H])c([2H])c6[2H])c5)nc(-c5c([2H])c([2H])c6c([2H])c([2H])c([2H])c([2H])c6c5[2H])n4)c3c2c1 ; [2H]c1cc2c(oc3c([2H])cc([2H])c(-c4nc(-c5ccc(-c6cccc7ccccc67)c(-c6ccccc6)c5)nc(-c5c([2H])c([2H])c(-c6c([2H])c([2H])c([2H])c([2H])c6[2H])c([2H])c5[2H])n4)c32)c([2H])c1[2H]]

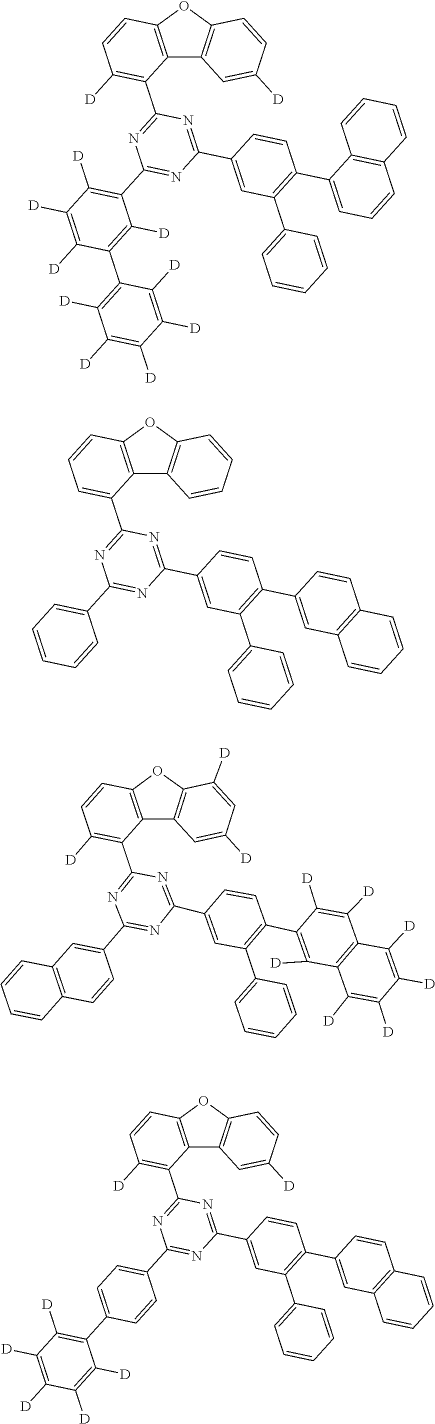

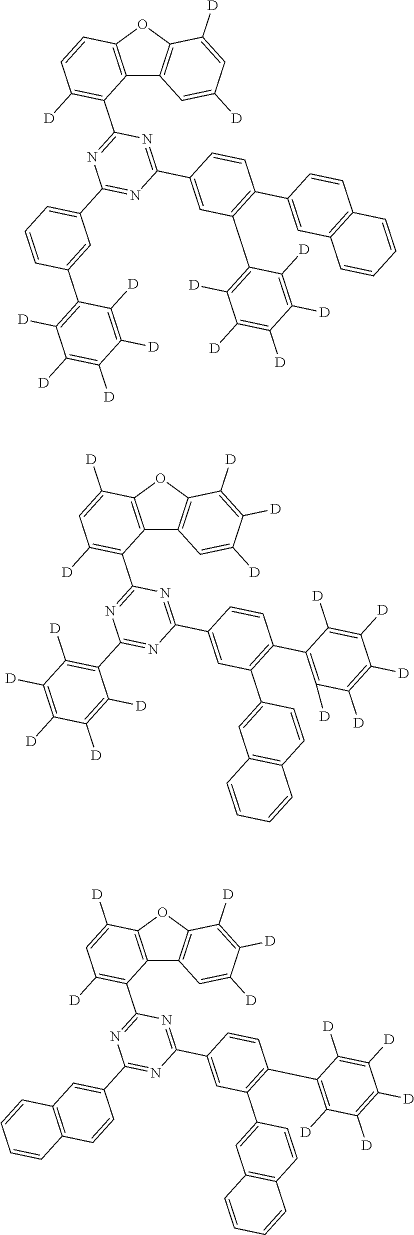 [SMILES: [2H]c1cc([2H])c2oc3ccc([2H])c(-c4nc(-c5cccc(-c6c([2H])c([2H])c([2H])c([2H])c6[2H])c5)nc(-c5ccc(-c6ccc7ccccc7c6)c(-c6c([2H])c([2H])c([2H])c([2H])c6[2H])c5)n4)c3c2c1 ; [2H]c1cc2c(oc3c([2H])cc([2H])c(-c4nc(-c5ccc(-c6c([2H])c([2H])c([2H])c([2H])c6[2H])c(-c6ccc7ccccc7c6)c5)nc(-c5c([2H])c([2H])c([2H])c([2H])c5[2H])n4)c32)c([2H])c1[2H] ; [2H]c1cc2c(oc3c([2H])cc([2H])c(-c4nc(-c5ccc(-c6c([2H])c([2H])c([2H])c([2H])c6[2H])c(-c6ccc7ccccc7c6)c5)nc(-c5ccc6ccccc6c5)n4)c32)c([2H])c1[2H]]

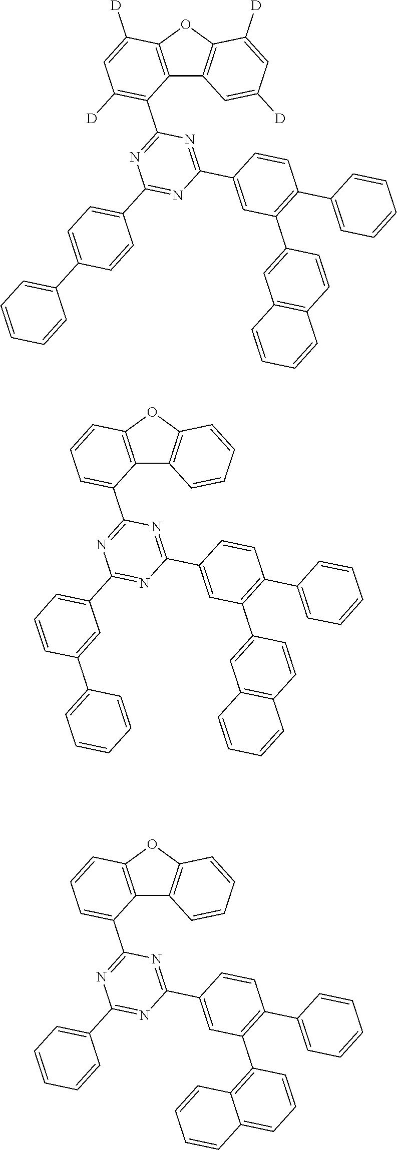 [SMILES: [2H]c1cc([2H])c2oc3c([2H])cc([2H])c(-c4nc(-c5ccc(-c6ccccc6)cc5)nc(-c5ccc(-c6ccccc6)c(-c6ccc7ccccc7c6)c5)n4)c3c2c1 ; c1ccc(-c2cccc(-c3nc(-c4ccc(-c5ccccc5)c(-c5ccc6ccccc6c5)c4)nc(-c4cccc5oc6ccccc6c45)n3)c2)cc1 ; c1ccc(-c2nc(-c3ccc(-c4ccccc4)c(-c4cccc5ccccc45)c3)nc(-c3cccc4oc5ccccc5c34)n2)cc1]

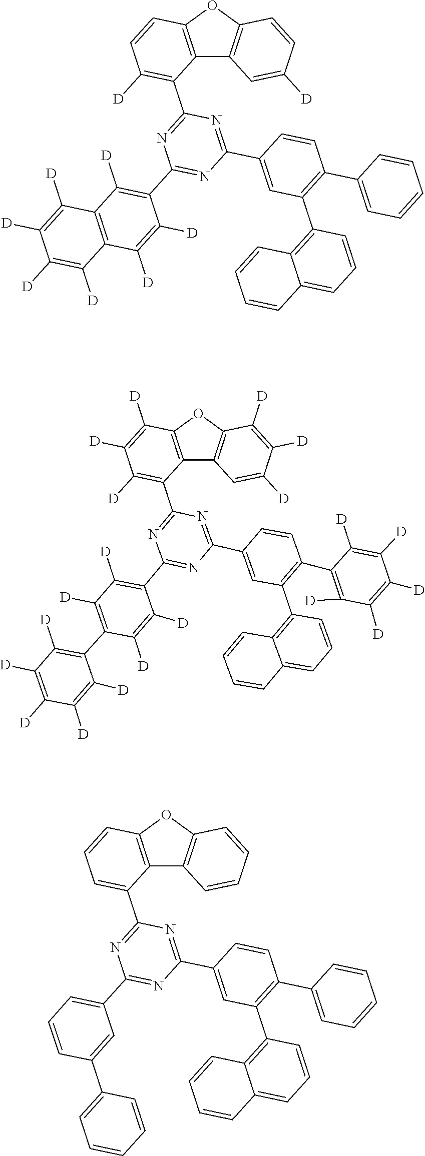 [SMILES: [2H]c1ccc2oc3ccc([2H])c(-c4nc(-c5ccc(-c6ccccc6)c(-c6cccc7ccccc67)c5)nc(-c5c([2H])c([2H])c6c([2H])c([2H])c([2H])c([2H])c6c5[2H])n4)c3c2c1 ; [2H]c1cc2c(oc3c([2H])c([2H])c([2H])c(-c4nc(-c5ccc(-c6c([2H])c([2H])c([2H])c([2H])c6[2H])c(-c6cccc7ccccc67)c5)nc(-c5c([2H])c([2H])c(-c6c([2H])c([2H])c([2H])c([2H])c6[2H])c([2H])c5[2H])n4)c32)c([2H])c1[2H] ; c1ccc(-c2cccc(-c3nc(-c4ccc(-c5ccccc5)c(-c5cccc6ccccc56)c4)nc(-c4cccc5oc6ccccc6c45)n3)c2)cc1]

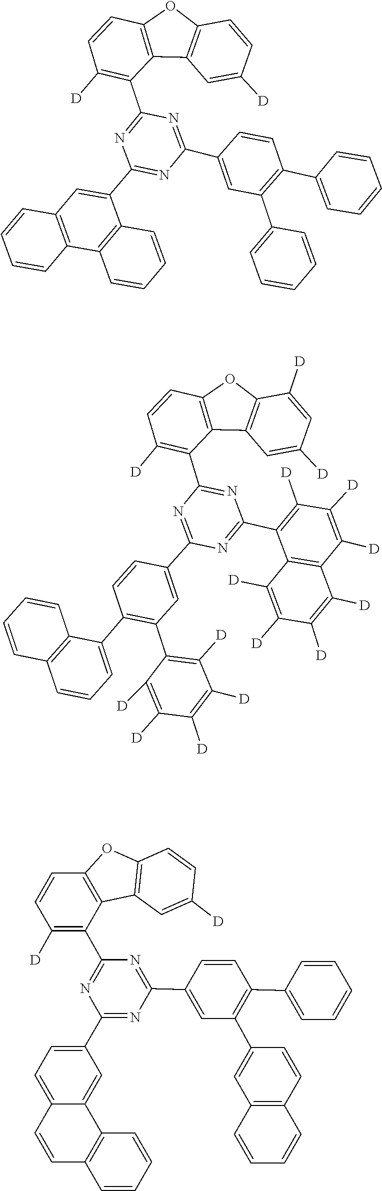 [SMILES: [2H]c1ccc2oc3ccc([2H])c(-c4nc(-c5ccc(-c6ccccc6)c(-c6ccccc6)c5)nc(-c5cc6ccccc6c6ccccc56)n4)c3c2c1 ; [2H]c1cc([2H])c2oc3ccc([2H])c(-c4nc(-c5ccc(-c6cccc7ccccc67)c(-c6c([2H])c([2H])c([2H])c([2H])c6[2H])c5)nc(-c5c([2H])c([2H])c([2H])c6c([2H])c([2H])c([2H])c([2H])c56)n4)c3c2c1 ; [2H]c1ccc2oc3ccc([2H])c(-c4nc(-c5ccc(-c6ccccc6)c(-c6ccc7ccccc7c6)c5)nc(-c5ccc6ccc7ccccc7c6c5)n4)c3c2c1]

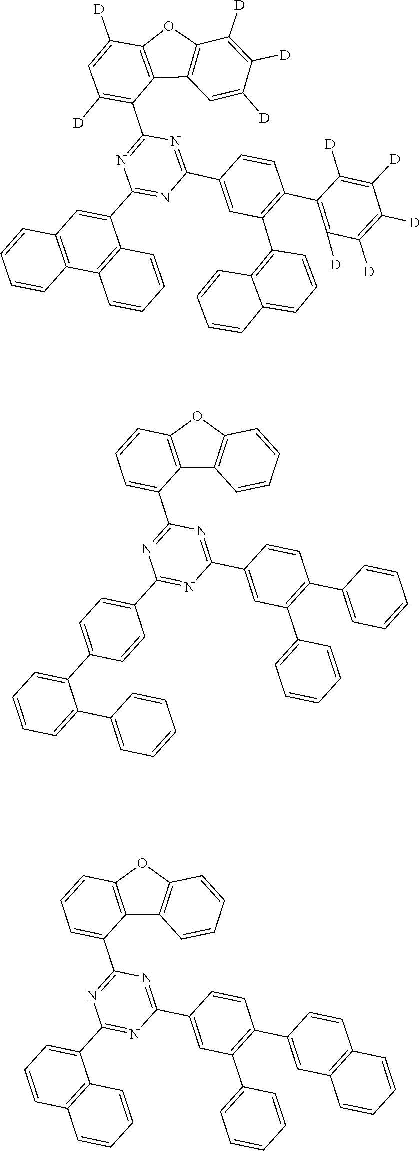 [SMILES: [2H]c1cc2c(oc3c([2H])cc([2H])c(-c4nc(-c5ccc(-c6c([2H])c([2H])c([2H])c([2H])c6[2H])c(-c6cccc7ccccc67)c5)nc(-c5cc6ccccc6c6ccccc56)n4)c32)c([2H])c1[2H] ; c1ccc(-c2ccccc2-c2ccc(-c3nc(-c4ccc(-c5ccccc5)c(-c5ccccc5)c4)nc(-c4cccc5oc6ccccc6c45)n3)cc2)cc1 ; c1ccc(-c2cc(-c3nc(-c4cccc5ccccc45)nc(-c4cccc5oc6ccccc6c45)n3)ccc2-c2ccc3ccccc3c2)cc1]

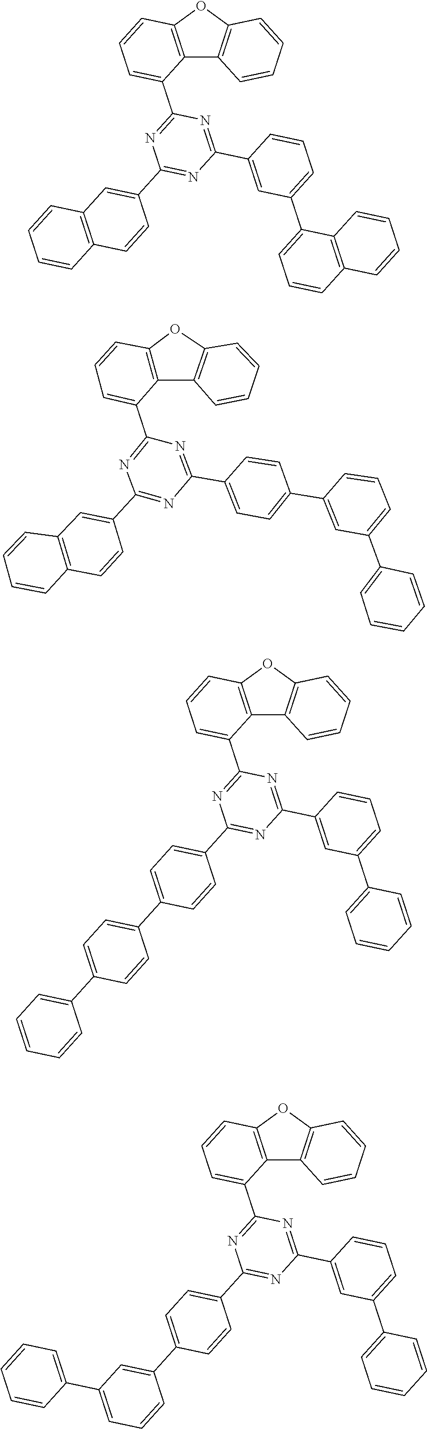 [SMILES: c1cc(-c2nc(-c3ccc4ccccc4c3)nc(-c3cccc4oc5ccccc5c34)n2)cc(-c2cccc3ccccc23)c1 ; c1ccc(-c2cccc(-c3ccc(-c4nc(-c5ccc6ccccc6c5)nc(-c5cccc6oc7ccccc7c56)n4)cc3)c2)cc1 ; c1ccc(-c2ccc(-c3ccc(-c4nc(-c5cccc(-c6ccccc6)c5)nc(-c5cccc6oc7ccccc7c56)n4)cc3)cc2)cc1 ; c1ccc(-c2cccc(-c3ccc(-c4nc(-c5cccc(-c6ccccc6)c5)nc(-c5cccc6oc7ccccc7c56)n4)cc3)c2)cc1]

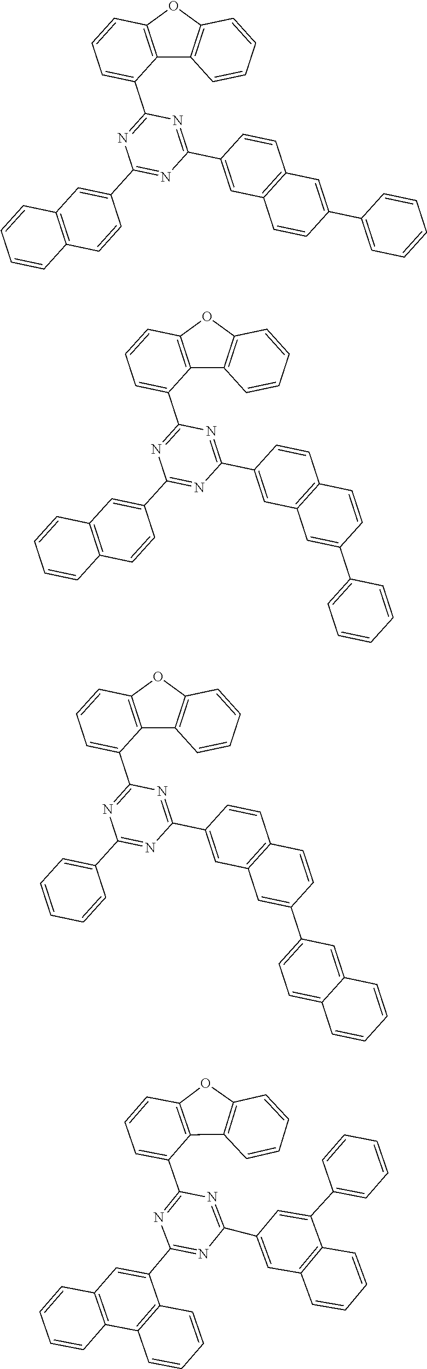 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc3ccc(-c4nc(-c5ccc6ccccc6c5)nc(-c5cccc6oc7ccccc7c56)n4)cc3c2)cc1 ; c1ccc(-c2nc(-c3ccc4ccc(-c5ccc6ccccc6c5)cc4c3)nc(-c3cccc4oc5ccccc5c34)n2)cc1 ; c1ccc(-c2cc(-c3nc(-c4cc5ccccc5c5ccccc45)nc(-c4cccc5oc6ccccc6c45)n3)cc3ccccc23)cc1]

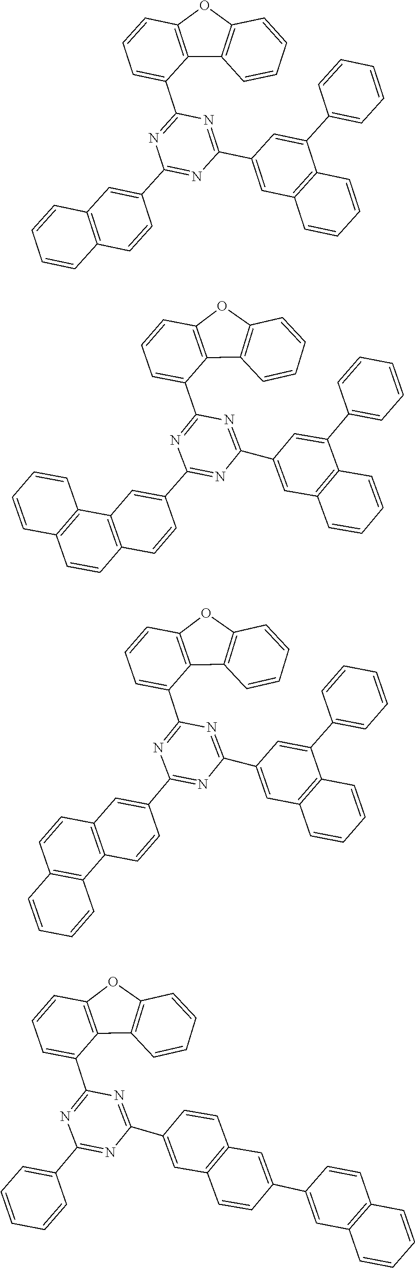 [SMILES: c1ccc(-c2cc(-c3nc(-c4ccc5ccccc5c4)nc(-c4cccc5oc6ccccc6c45)n3)cc3ccccc23)cc1 ; c1ccc(-c2cc(-c3nc(-c4ccc5ccc6ccccc6c5c4)nc(-c4cccc5oc6ccccc6c45)n3)cc3ccccc23)cc1 ; c1ccc(-c2cc(-c3nc(-c4ccc5c(ccc6ccccc65)c4)nc(-c4cccc5oc6ccccc6c45)n3)cc3ccccc23)cc1 ; c1ccc(-c2nc(-c3ccc4cc(-c5ccc6ccccc6c5)ccc4c3)nc(-c3cccc4oc5ccccc5c34)n2)cc1]

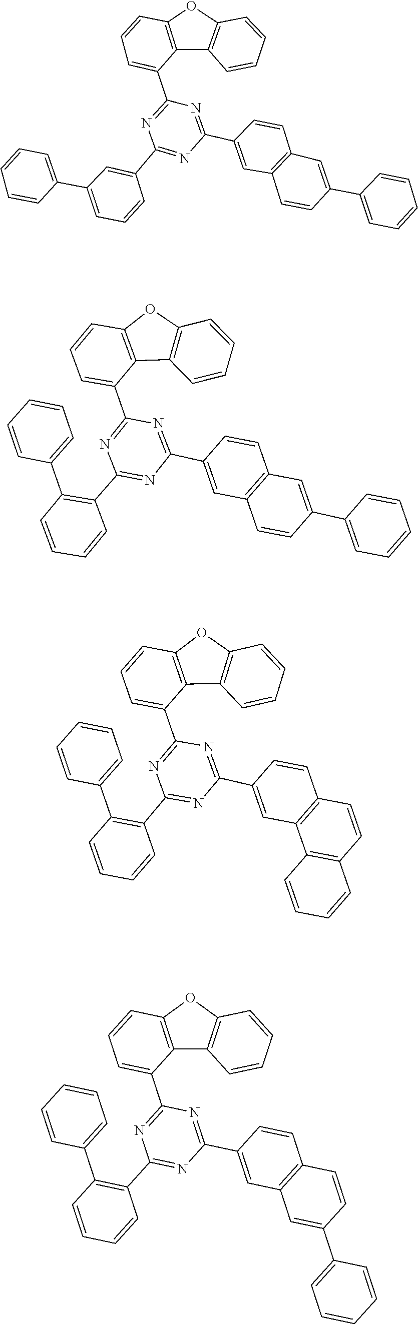 [SMILES: c1ccc(-c2cccc(-c3nc(-c4ccc5cc(-c6ccccc6)ccc5c4)nc(-c4cccc5oc6ccccc6c45)n3)c2)cc1 ; c1ccc(-c2ccc3cc(-c4nc(-c5ccccc5-c5ccccc5)nc(-c5cccc6oc7ccccc7c56)n4)ccc3c2)cc1 ; c1ccc(-c2ccccc2-c2nc(-c3ccc4ccc5ccccc5c4c3)nc(-c3cccc4oc5ccccc5c34)n2)cc1 ; c1ccc(-c2ccc3ccc(-c4nc(-c5ccccc5-c5ccccc5)nc(-c5cccc6oc7ccccc7c56)n4)cc3c2)cc1]

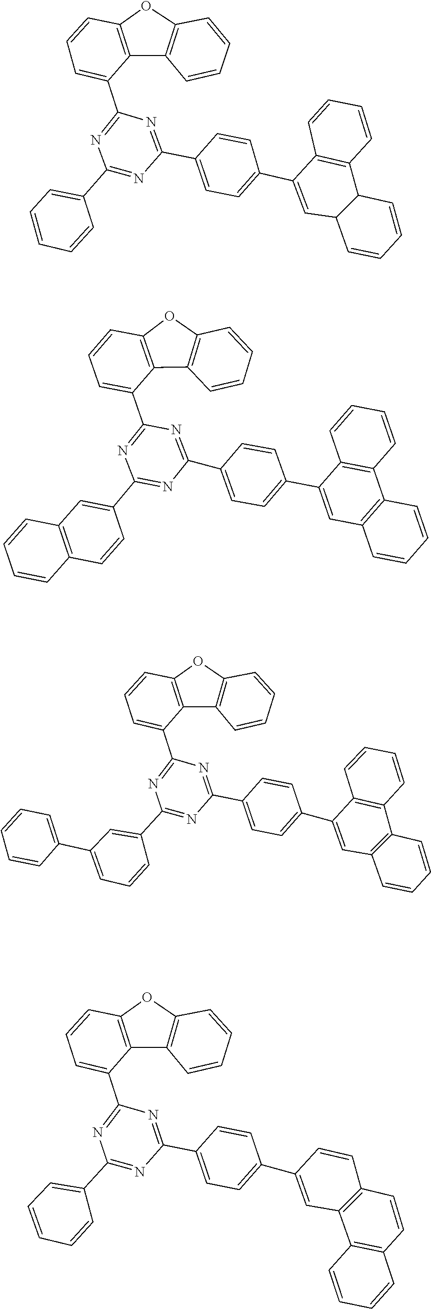 [SMILES: C1=CC2C=C(c3ccc(-c4nc(-c5ccccc5)nc(-c5cccc6oc7ccccc7c56)n4)cc3)c3ccccc3C2C=C1 ; c1ccc2cc(-c3nc(-c4ccc(-c5cc6ccccc6c6ccccc56)cc4)nc(-c4cccc5oc6ccccc6c45)n3)ccc2c1 ; c1ccc(-c2cccc(-c3nc(-c4ccc(-c5cc6ccccc6c6ccccc56)cc4)nc(-c4cccc5oc6ccccc6c45)n3)c2)cc1 ; c1ccc(-c2nc(-c3ccc(-c4ccc5ccc6ccccc6c5c4)cc3)nc(-c3cccc4oc5ccccc5c34)n2)cc1]

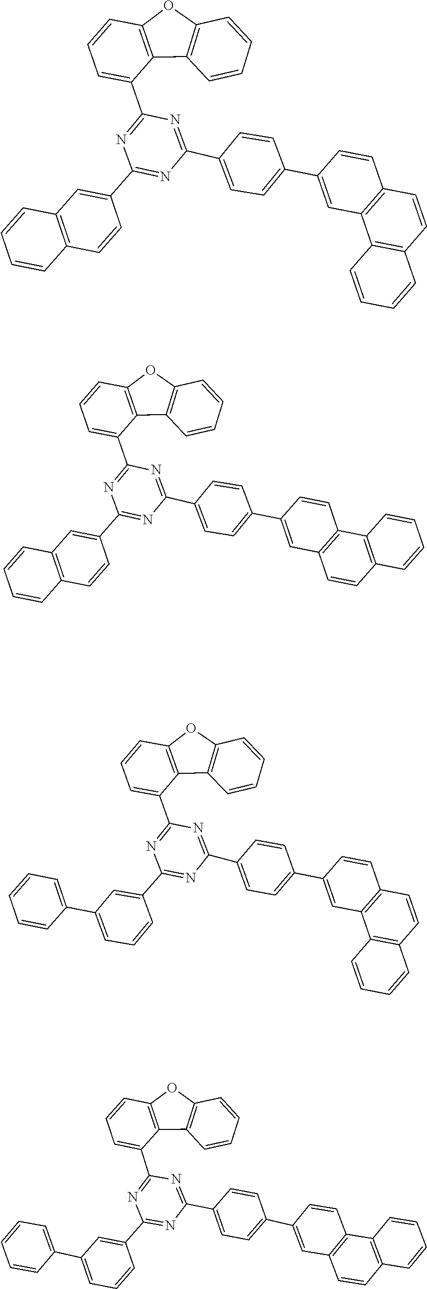 [SMILES: c1ccc2cc(-c3nc(-c4ccc(-c5ccc6ccc7ccccc7c6c5)cc4)nc(-c4cccc5oc6ccccc6c45)n3)ccc2c1 ; c1ccc2cc(-c3nc(-c4ccc(-c5ccc6c(ccc7ccccc76)c5)cc4)nc(-c4cccc5oc6ccccc6c45)n3)ccc2c1 ; c1ccc(-c2cccc(-c3nc(-c4ccc(-c5ccc6ccc7ccccc7c6c5)cc4)nc(-c4cccc5oc6ccccc6c45)n3)c2)cc1 ; c1ccc(-c2cccc(-c3nc(-c4ccc(-c5ccc6c(ccc7ccccc76)c5)cc4)nc(-c4cccc5oc6ccccc6c45)n3)c2)cc1]

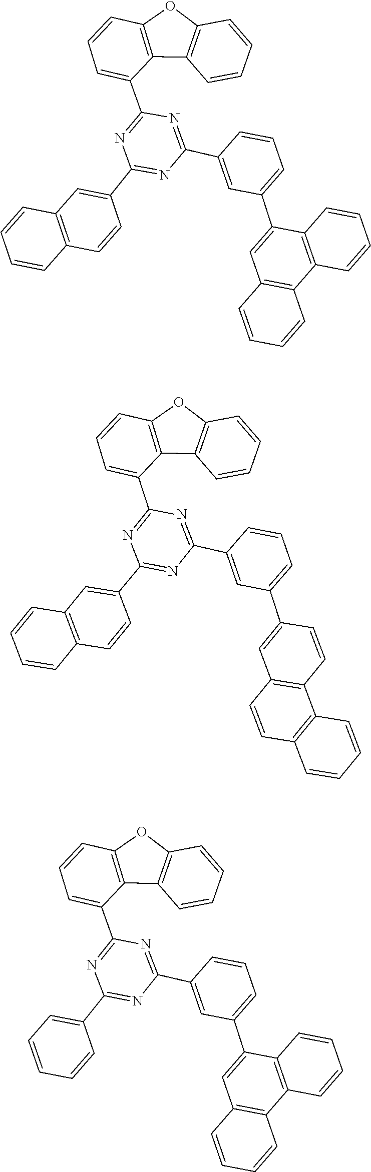 [SMILES: c1cc(-c2nc(-c3ccc4ccccc4c3)nc(-c3cccc4oc5ccccc5c34)n2)cc(-c2cc3ccccc3c3ccccc23)c1 ; c1cc(-c2ccc3c(ccc4ccccc43)c2)cc(-c2nc(-c3ccc4ccccc4c3)nc(-c3cccc4oc5ccccc5c34)n2)c1 ; c1ccc(-c2nc(-c3cccc(-c4cc5ccccc5c5ccccc45)c3)nc(-c3cccc4oc5ccccc5c34)n2)cc1]

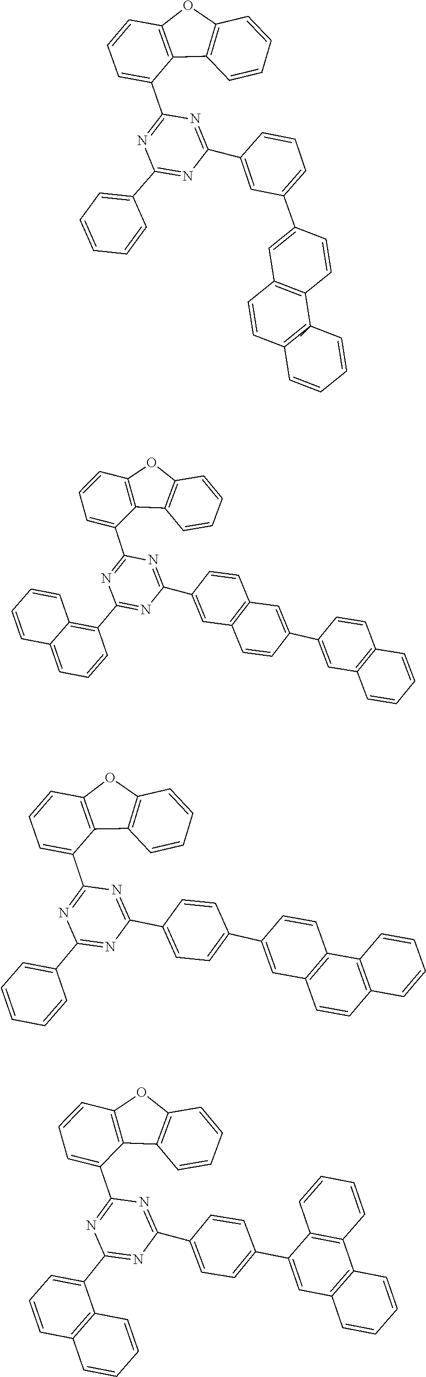

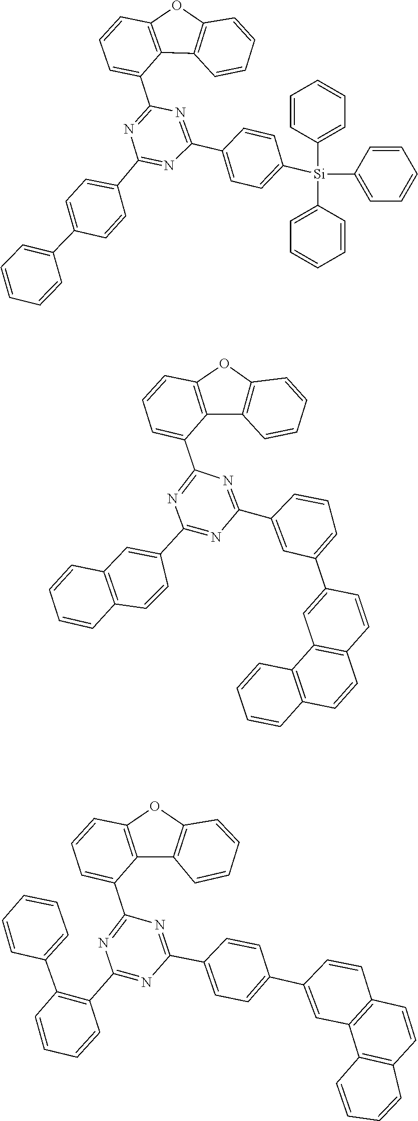 [SMILES: c1ccc(-c2ccc(-c3nc(-c4ccc([Si](c5ccccc5)(c5ccccc5)c5ccccc5)cc4)nc(-c4cccc5oc6ccccc6c45)n3)cc2)cc1 ; c1cc(-c2ccc3ccc4ccccc4c3c2)cc(-c2nc(-c3ccc4ccccc4c3)nc(-c3cccc4oc5ccccc5c34)n2)c1 ; c1ccc(-c2ccccc2-c2nc(-c3ccc(-c4ccc5ccc6ccccc6c5c4)cc3)nc(-c3cccc4oc5ccccc5c34)n2)cc1]

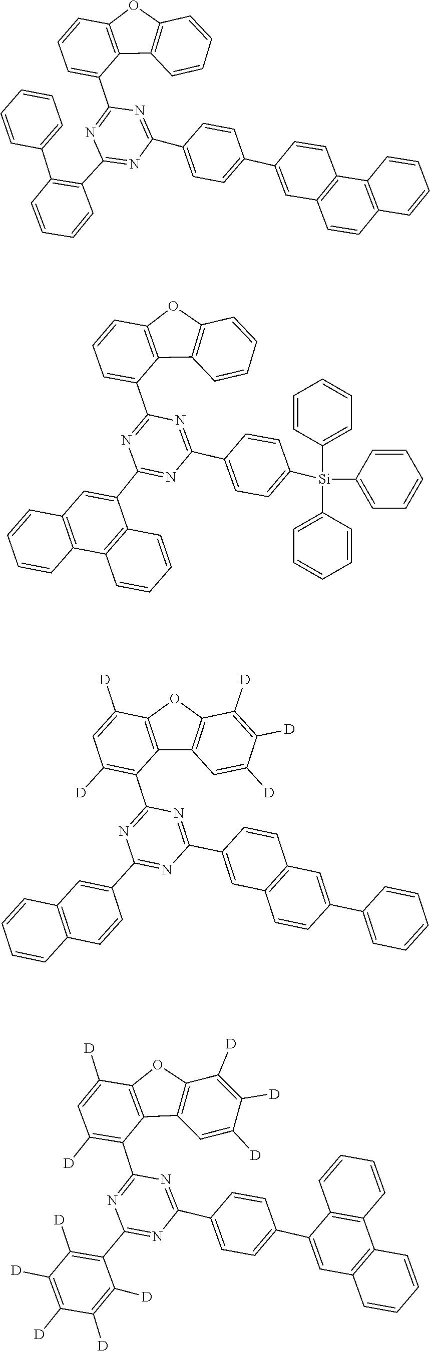 [SMILES: c1ccc(-c2ccccc2-c2nc(-c3ccc(-c4ccc5c(ccc6ccccc65)c4)cc3)nc(-c3cccc4oc5ccccc5c34)n2)cc1 ; c1ccc([Si](c2ccccc2)(c2ccccc2)c2ccc(-c3nc(-c4cc5ccccc5c5ccccc45)nc(-c4cccc5oc6ccccc6c45)n3)cc2)cc1 ; [2H]c1cc2c(oc3c([2H])cc([2H])c(-c4nc(-c5ccc6ccccc6c5)nc(-c5ccc6cc(-c7ccccc7)ccc6c5)n4)c32)c([2H])c1[2H] ; [2H]c1cc2c(oc3c([2H])cc([2H])c(-c4nc(-c5ccc(-c6cc7ccccc7c7ccccc67)cc5)nc(-c5c([2H])c([2H])c([2H])c([2H])c5[2H])n4)c32)c([2H])c1[2H]]

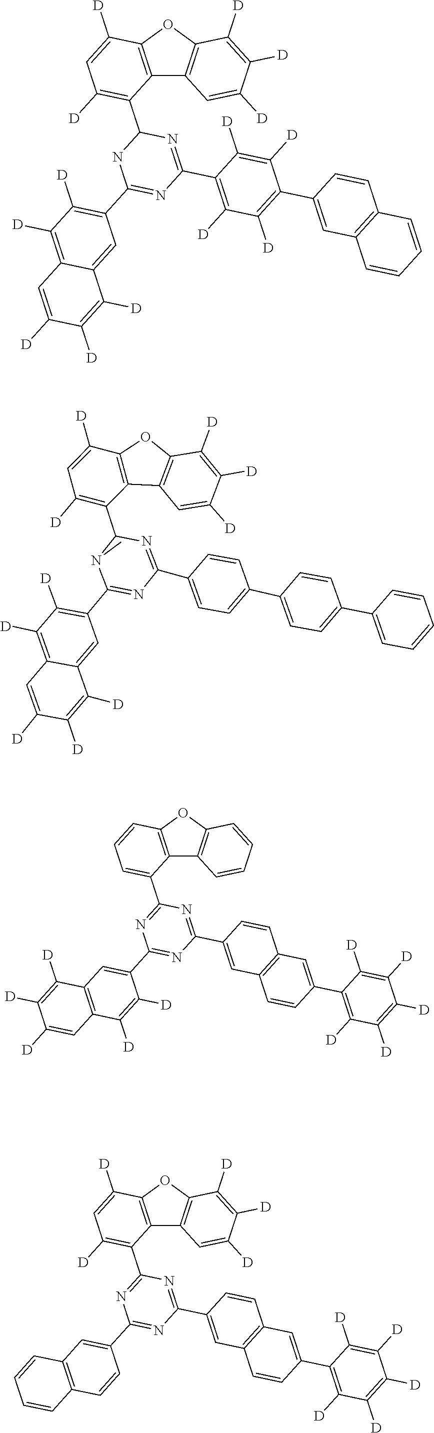

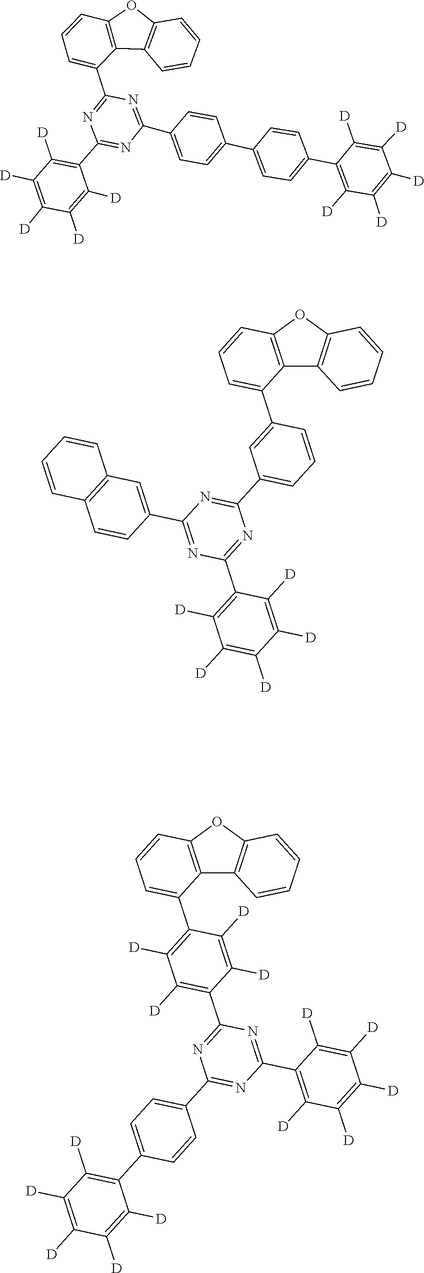 [SMILES: [2H]c1c([2H])c([2H])c(-c2ccc(-c3ccc(-c4nc(-c5c([2H])c([2H])c([2H])c([2H])c5[2H])nc(-c5cccc6oc7ccccc7c56)n4)cc3)cc2)c([2H])c1[2H] ; [2H]c1c([2H])c([2H])c(-c2nc(-c3cccc(-c4cccc5oc6ccccc6c45)c3)nc(-c3ccc4ccccc4c3)n2)c([2H])c1[2H] ; [2H]c1c([2H])c([2H])c(-c2ccc(-c3nc(-c4c([2H])c([2H])c([2H])c([2H])c4[2H])nc(-c4c([2H])c([2H])c(-c5cccc6oc7ccccc7c56)c([2H])c4[2H])n3)cc2)c([2H])c1[2H]]

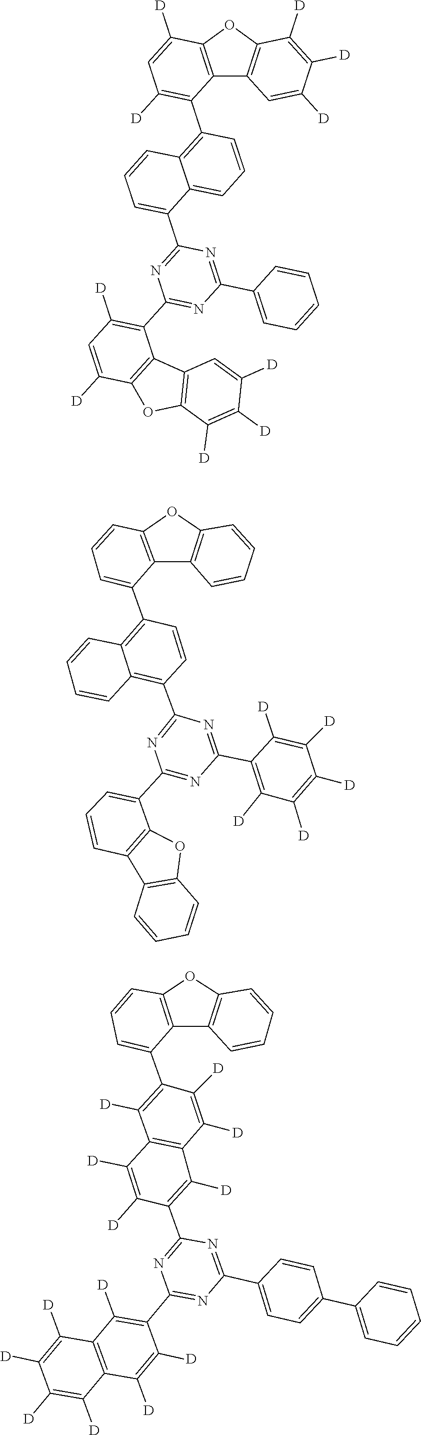

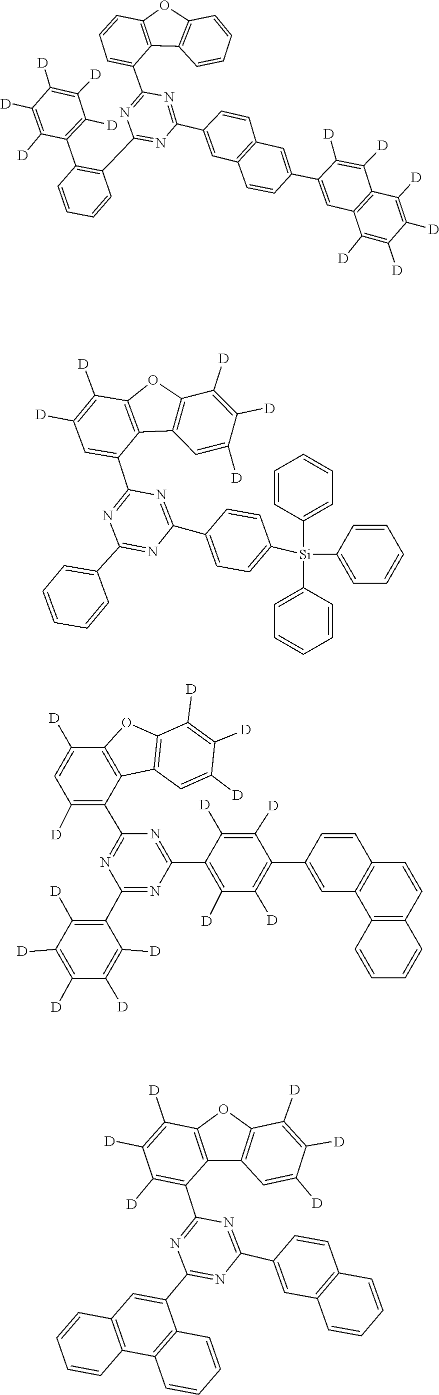

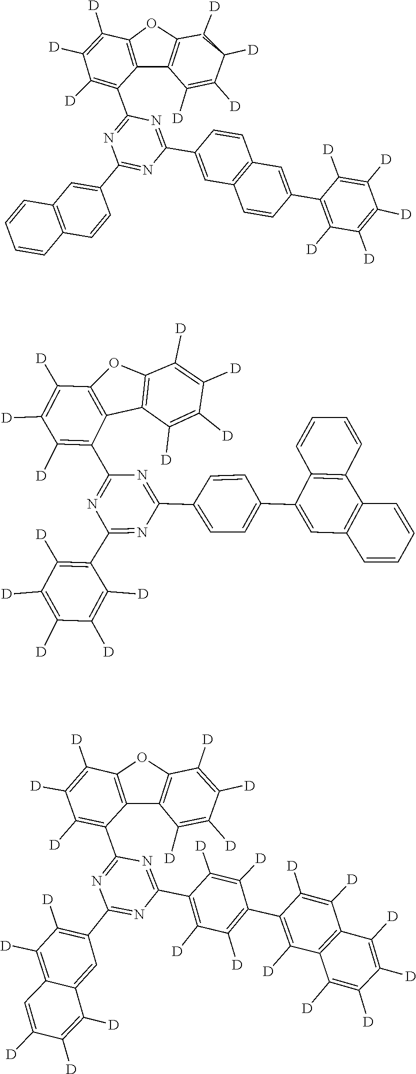

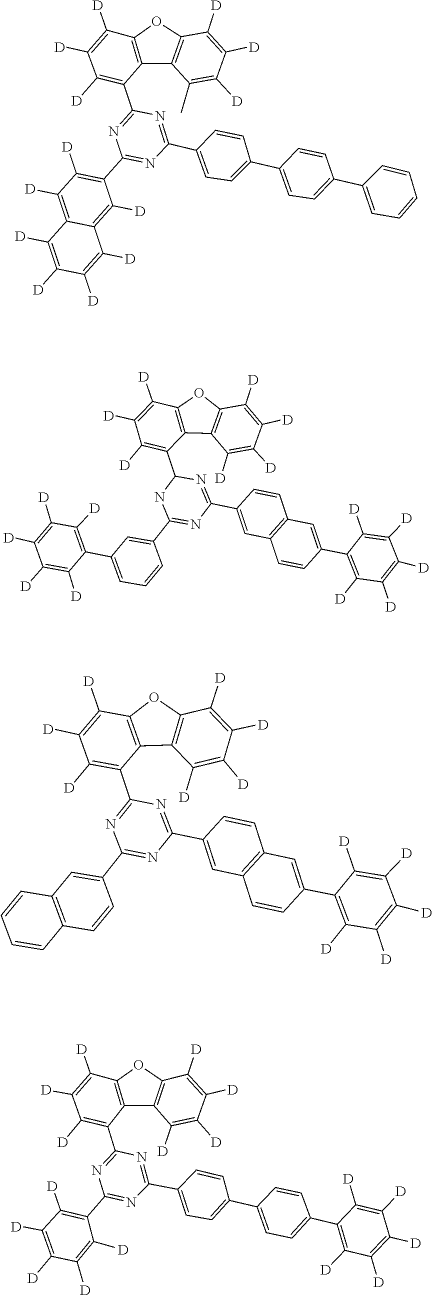

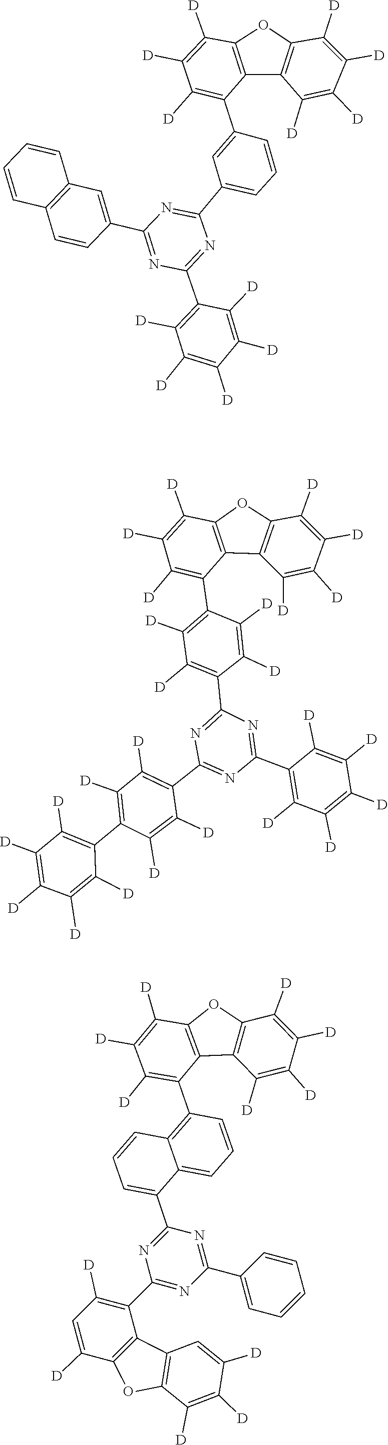

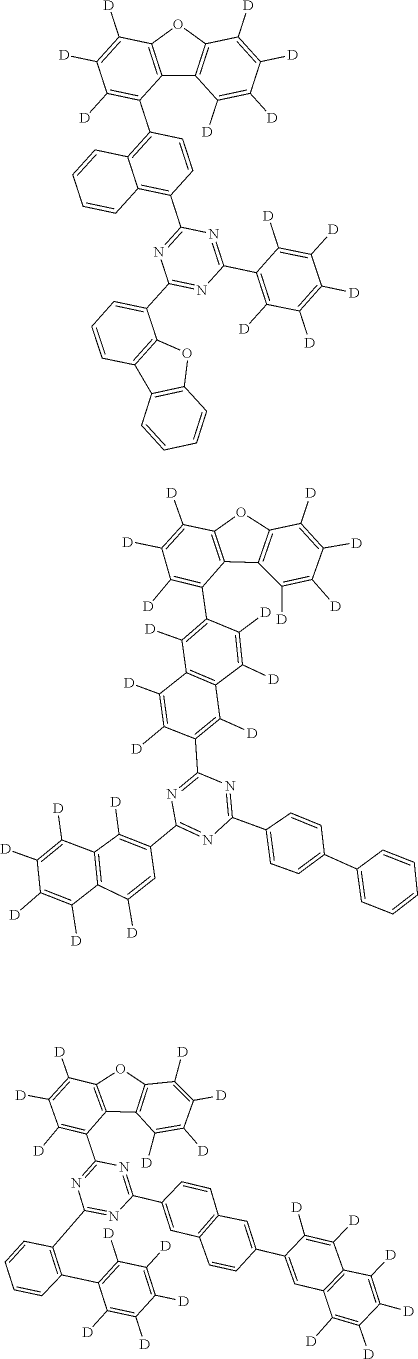

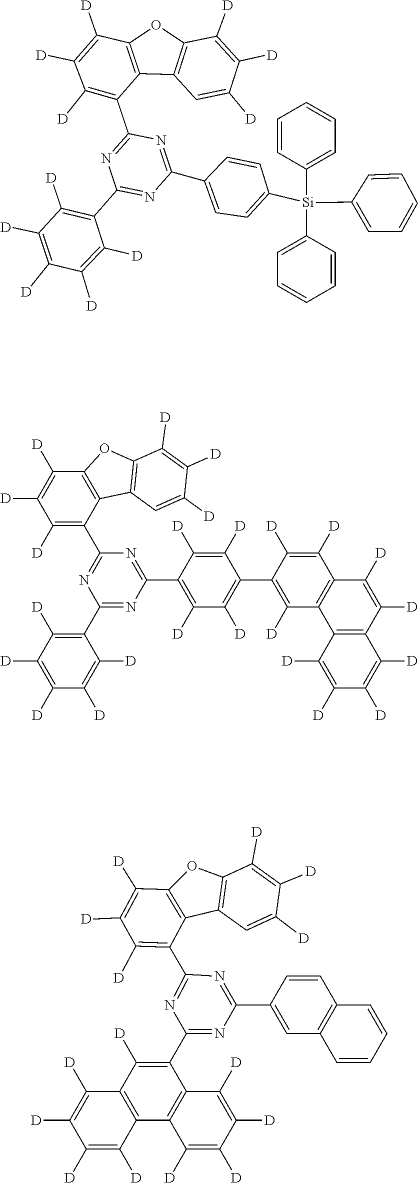

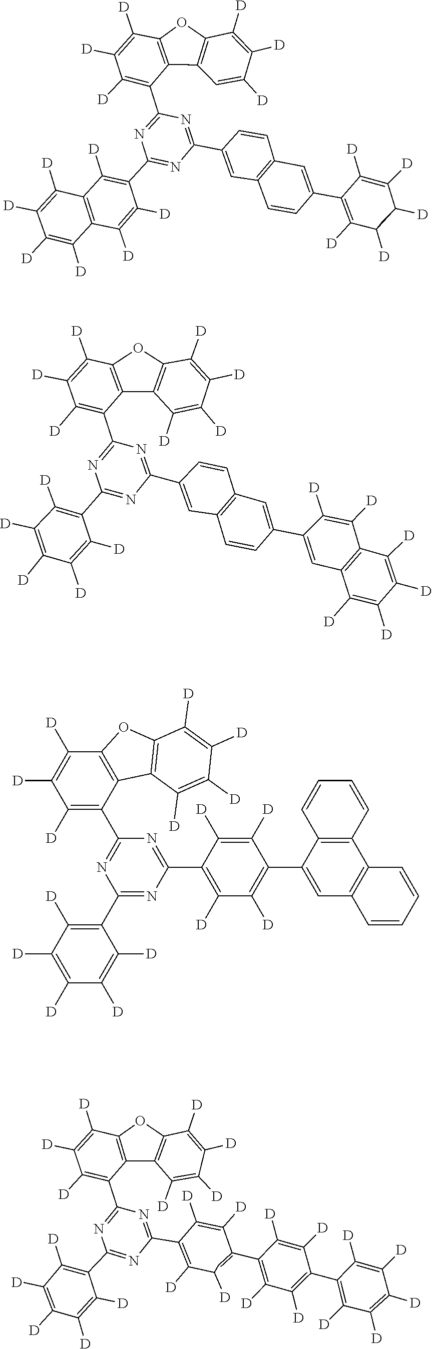

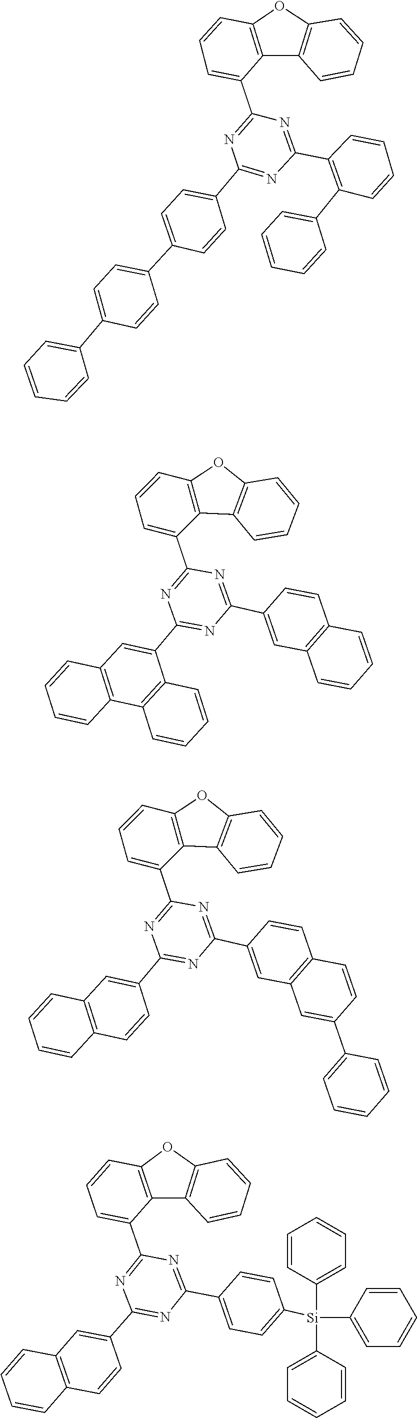 [SMILES: c1ccc(-c2ccc(-c3ccc(-c4nc(-c5ccccc5-c5ccccc5)nc(-c5cccc6oc7ccccc7c56)n4)cc3)cc2)cc1 ; c1ccc2cc(-c3nc(-c4cc5ccccc5c5ccccc45)nc(-c4cccc5oc6ccccc6c45)n3)ccc2c1 ; c1ccc(-c2ccc3ccc(-c4nc(-c5ccc6ccccc6c5)nc(-c5cccc6oc7ccccc7c56)n4)cc3c2)cc1 ; c1ccc([Si](c2ccccc2)(c2ccccc2)c2ccc(-c3nc(-c4ccc5ccccc5c4)nc(-c4cccc5oc6ccccc6c45)n3)cc2)cc1]

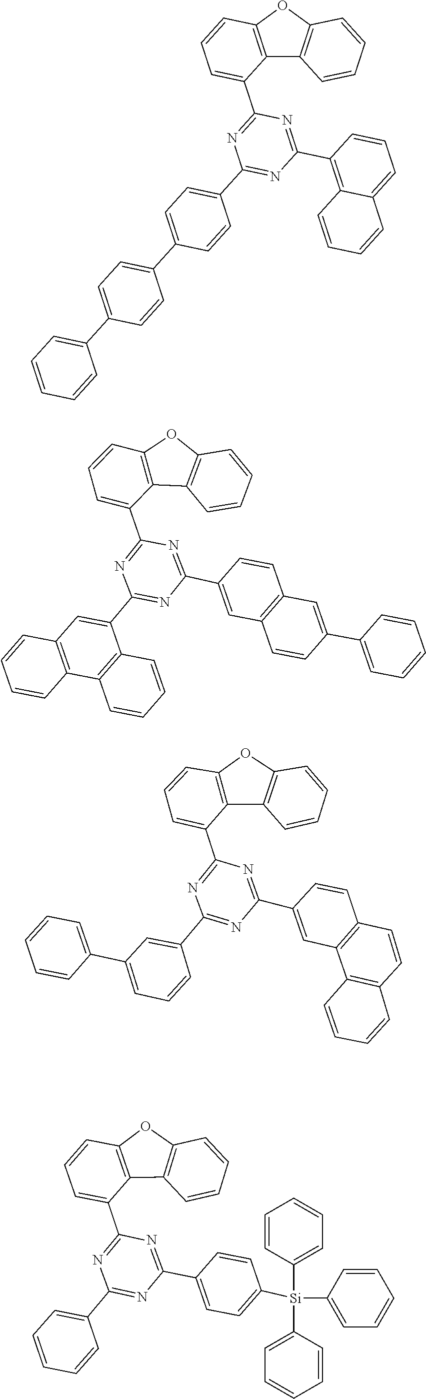 [SMILES: c1ccc(-c2ccc(-c3ccc(-c4nc(-c5cccc6ccccc56)nc(-c5cccc6oc7ccccc7c56)n4)cc3)cc2)cc1 ; c1ccc(-c2ccc3cc(-c4nc(-c5cc6ccccc6c6ccccc56)nc(-c5cccc6oc7ccccc7c56)n4)ccc3c2)cc1 ; c1ccc(-c2cccc(-c3nc(-c4ccc5ccc6ccccc6c5c4)nc(-c4cccc5oc6ccccc6c45)n3)c2)cc1 ; c1ccc(-c2nc(-c3ccc([Si](c4ccccc4)(c4ccccc4)c4ccccc4)cc3)nc(-c3cccc4oc5ccccc5c34)n2)cc1]

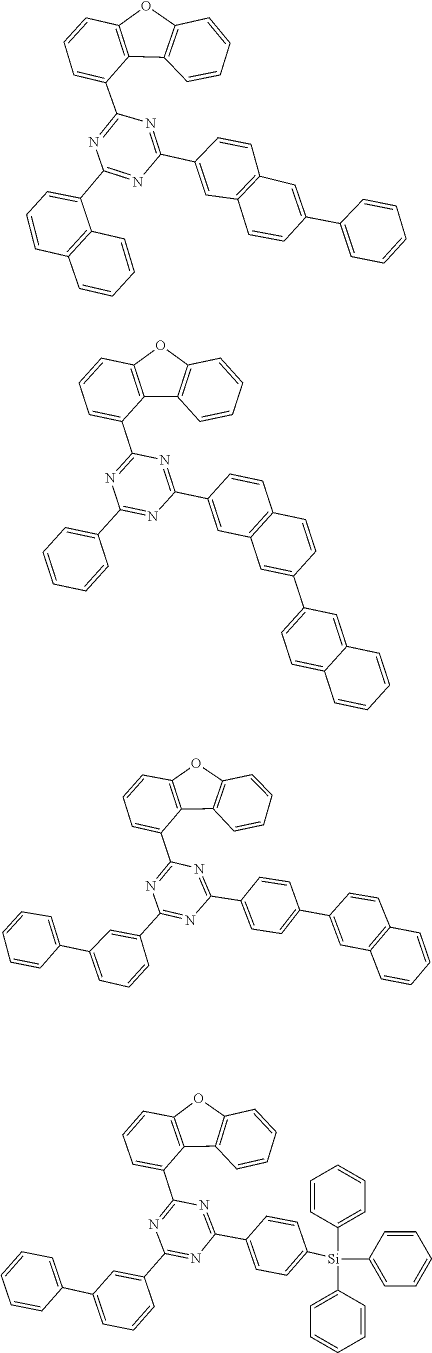 [SMILES: c1ccc(-c2ccc3cc(-c4nc(-c5cccc6ccccc56)nc(-c5cccc6oc7ccccc7c56)n4)ccc3c2)cc1 ; c1ccc(-c2nc(-c3ccc4ccc(-c5ccc6ccccc6c5)cc4c3)nc(-c3cccc4oc5ccccc5c34)n2)cc1 ; c1ccc(-c2cccc(-c3nc(-c4ccc(-c5ccc6ccccc6c5)cc4)nc(-c4cccc5oc6ccccc6c45)n3)c2)cc1 ; c1ccc(-c2cccc(-c3nc(-c4ccc([Si](c5ccccc5)(c5ccccc5)c5ccccc5)cc4)nc(-c4cccc5oc6ccccc6c45)n3)c2)cc1]

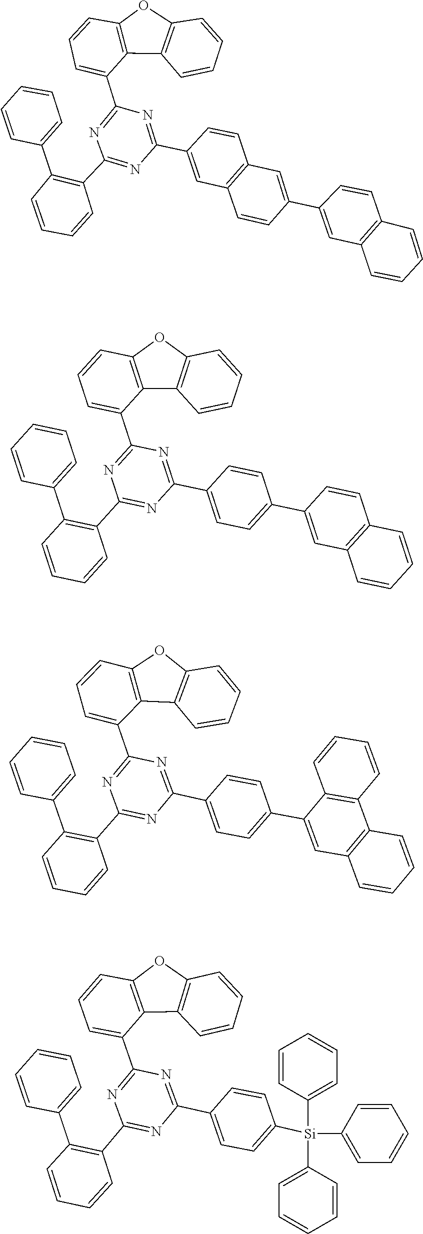 [SMILES: c1ccc(-c2ccccc2-c2nc(-c3ccc4cc(-c5ccc6ccccc6c5)ccc4c3)nc(-c3cccc4oc5ccccc5c34)n2)cc1 ; c1ccc(-c2ccccc2-c2nc(-c3ccc(-c4ccc5ccccc5c4)cc3)nc(-c3cccc4oc5ccccc5c34)n2)cc1 ; c1ccc(-c2ccccc2-c2nc(-c3ccc(-c4cc5ccccc5c5ccccc45)cc3)nc(-c3cccc4oc5ccccc5c34)n2)cc1 ; c1ccc(-c2ccccc2-c2nc(-c3ccc([Si](c4ccccc4)(c4ccccc4)c4ccccc4)cc3)nc(-c3cccc4oc5ccccc5c34)n2)cc1]

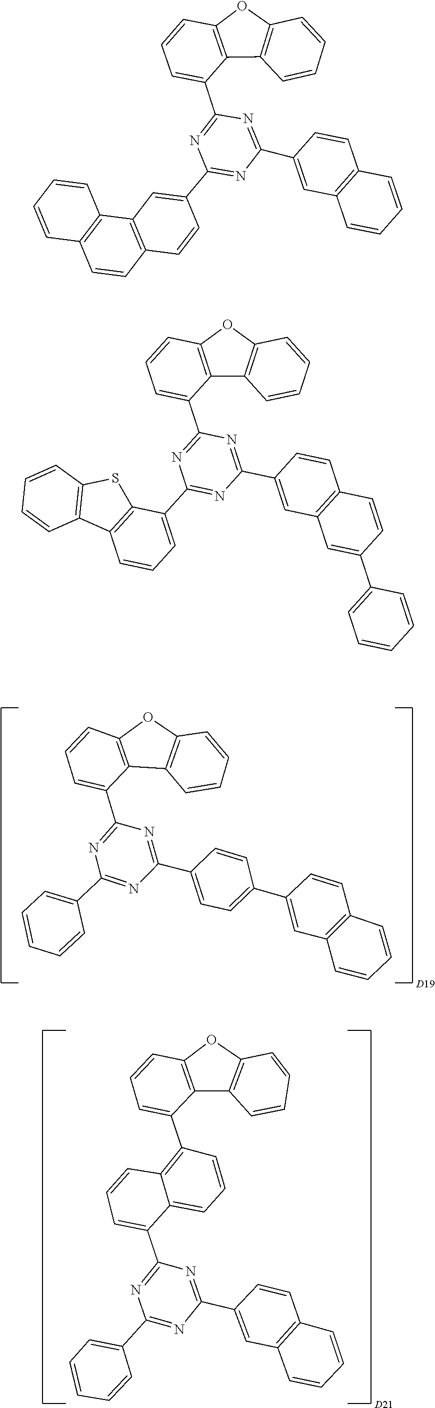

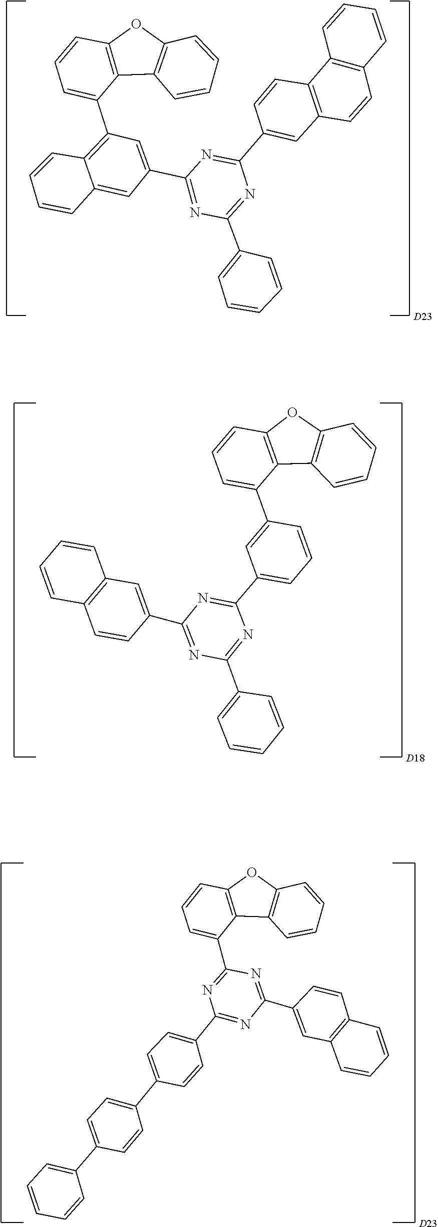

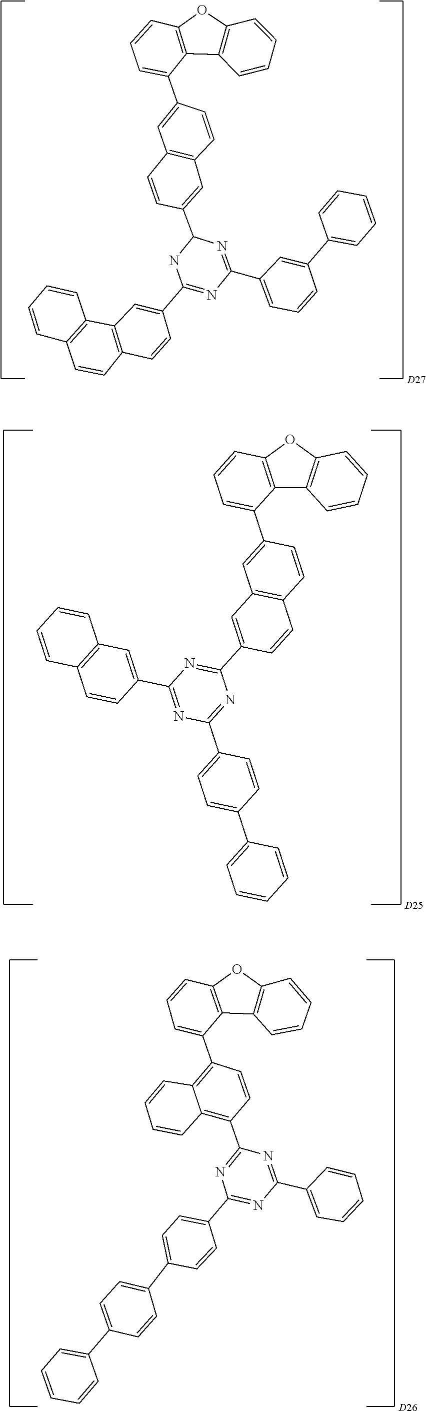

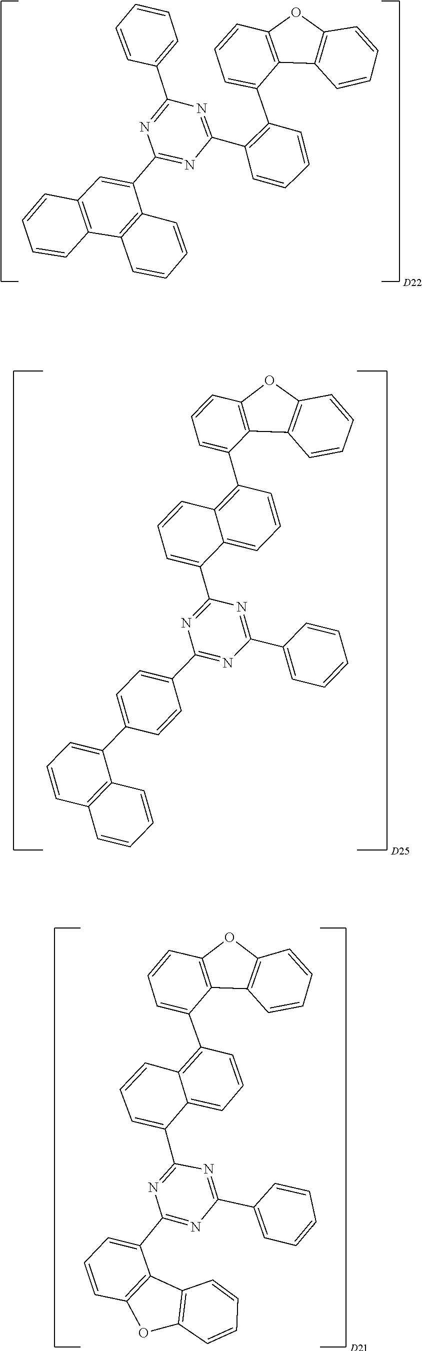

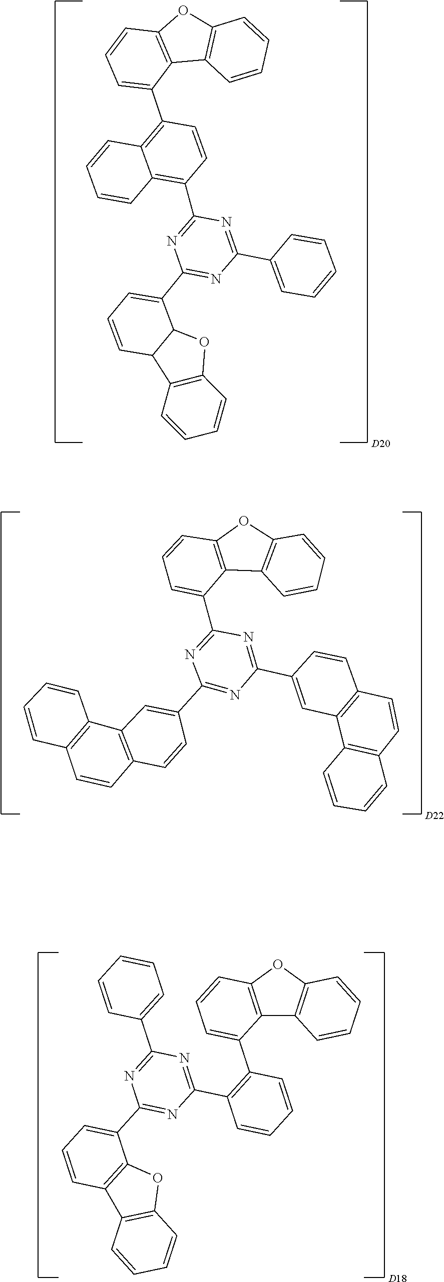

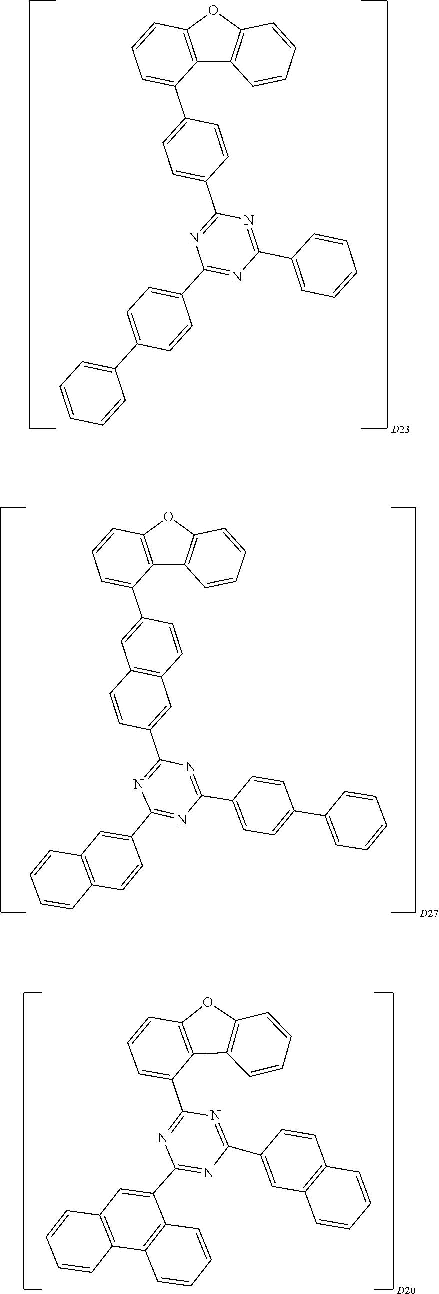

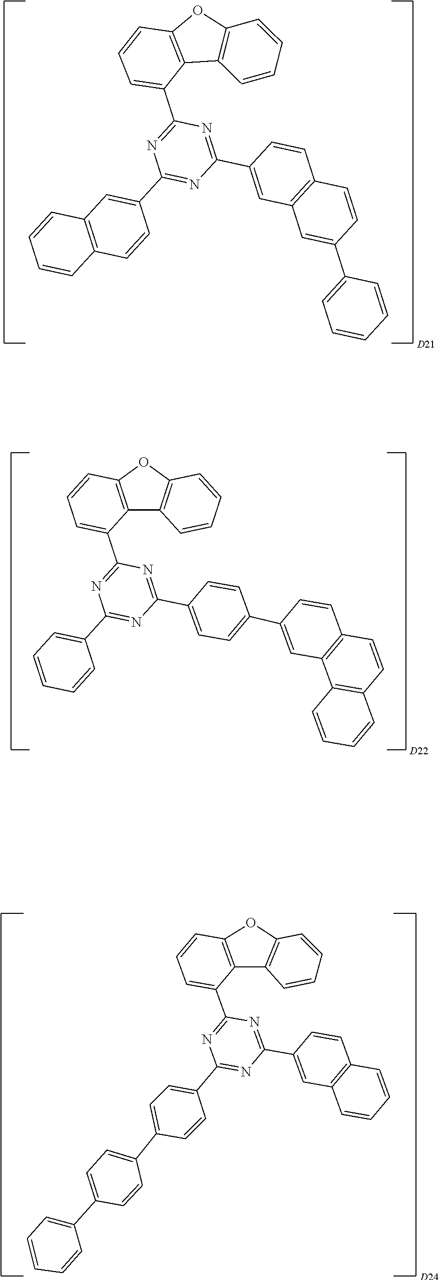

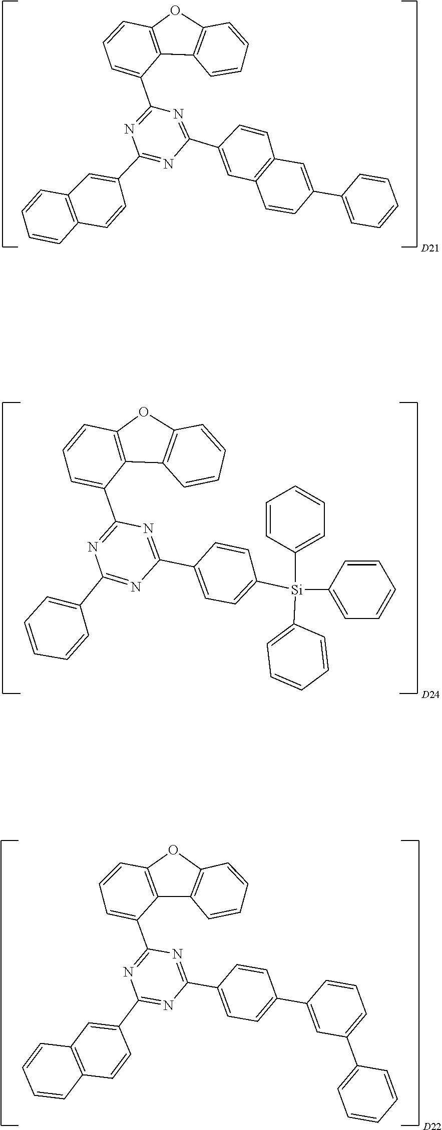

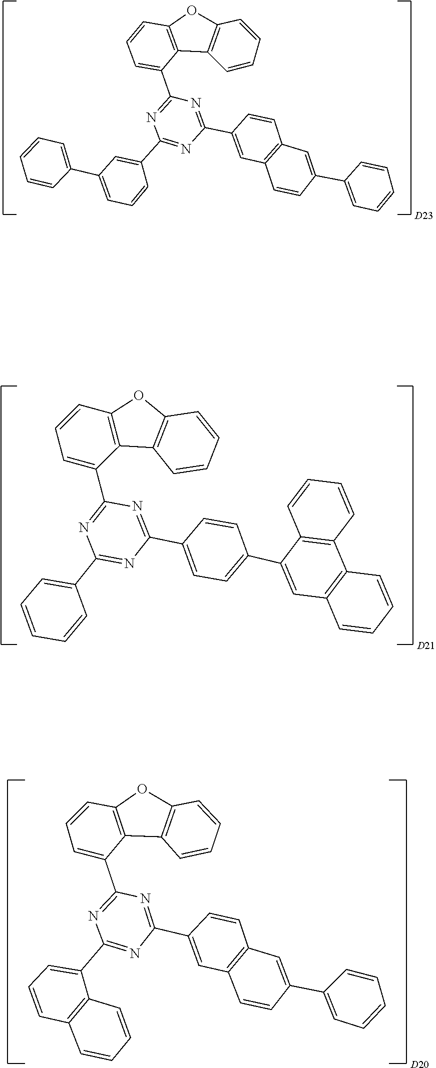

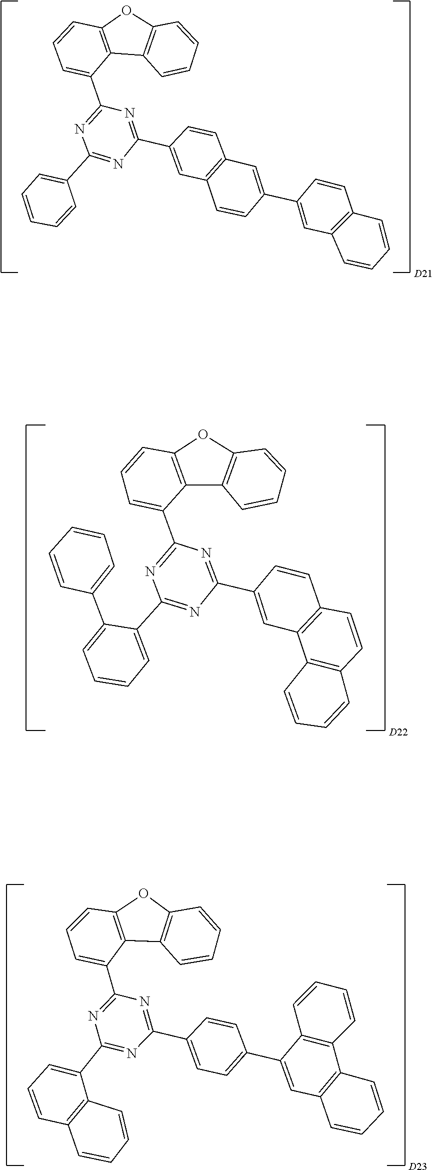

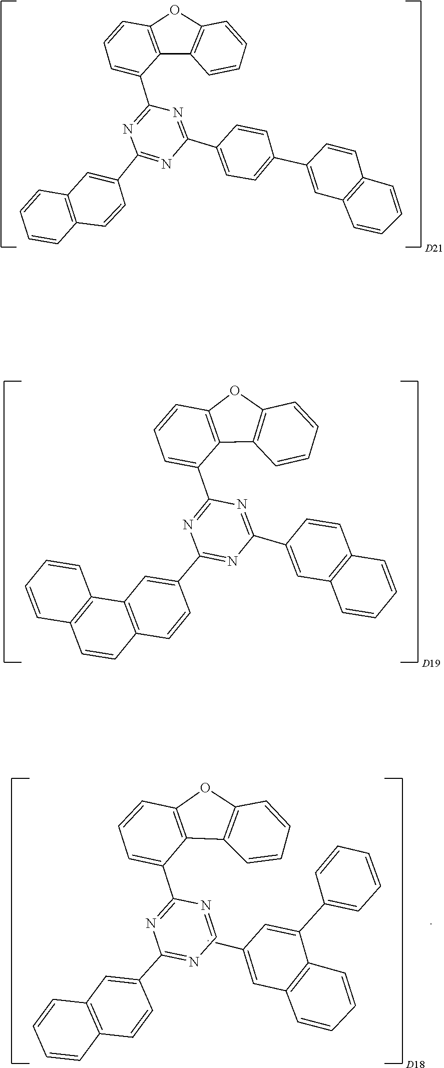

6. The organic light emitting device according to claim 1, wherein:
Ar₃ and Ar₄ are each independently phenyl, triphenylsilyl phenyl, biphenylyl, terphenylyl, naphthyl, phenyl naphthyl, phenanthrenyl, dibenzofuranyl, dibenzothiophenyl, phenyl carbazolyl, or dimethylfluorenyl; and
one or more hydrogens of Ar₃ and Ar₄ are each independently replaced with deuterium.

7. The organic light emitting device according to claim 1, wherein:
L₄ is phenylene, biphenyldiyl, biphenyldiyl substituted with phenyl, or naphthalenediyl; and
one or more of the hydrogens of L₄ are each independently replaced with deuterium.

8. The organic light emitting device according to claim 1, wherein:
L₅ and L₆ are each independently a single bond, phenylene, biphenyldiyl, naphthalenediyl, or carbazolediyl, and
one or more of the hydrogens of L₅ and L₆ each independently replaced with deuterium.

9. The organic light emitting device according to claim 1, wherein:
the compound of Chemical Formula 2 is any one compound selected from the group consisting of: 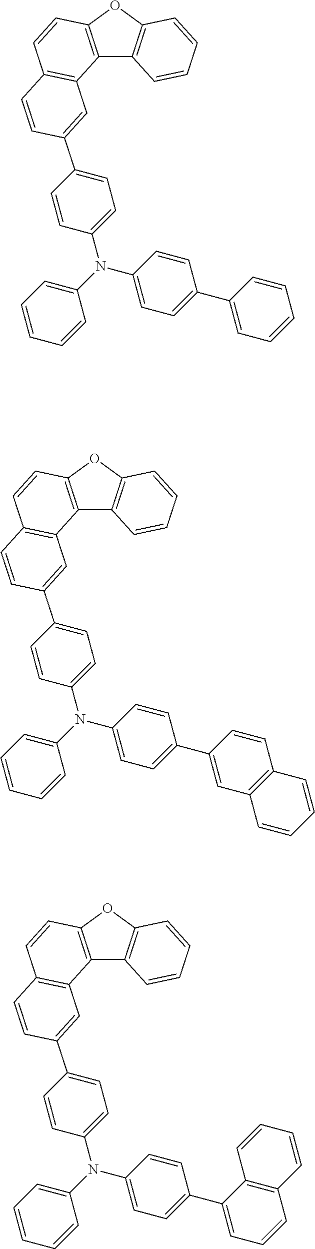 [SMILES: c1ccc(-c2ccc(N(c3ccccc3)c3ccc(-c4ccc5ccc6oc7ccccc7c6c5c4)cc3)cc2)cc1 ; c1ccc(N(c2ccc(-c3ccc4ccccc4c3)cc2)c2ccc(-c3ccc4ccc5oc6ccccc6c5c4c3)cc2)cc1 ; c1ccc(N(c2ccc(-c3ccc4ccc5oc6ccccc6c5c4c3)cc2)c2ccc(-c3cccc4ccccc34)cc2)cc1] 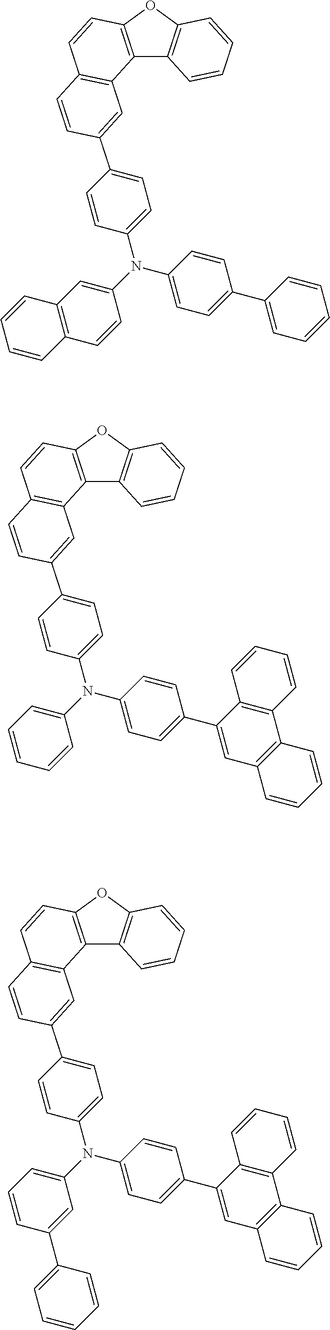 [SMILES: c1ccc(-c2ccc(N(c3ccc(-c4ccc5ccc6oc7ccccc7c6c5c4)cc3)c3ccc4ccccc4c3)cc2)cc1 ; c1ccc(N(c2ccc(-c3ccc4ccc5oc6ccccc6c5c4c3)cc2)c2ccc(-c3cc4ccccc4c4ccccc34)cc2)cc1 ; c1ccc(-c2cccc(N(c3ccc(-c4ccc5ccc6oc7ccccc7c6c5c4)cc3)c3ccc(-c4cc5ccccc5c5ccccc45)cc3)c2)cc1] 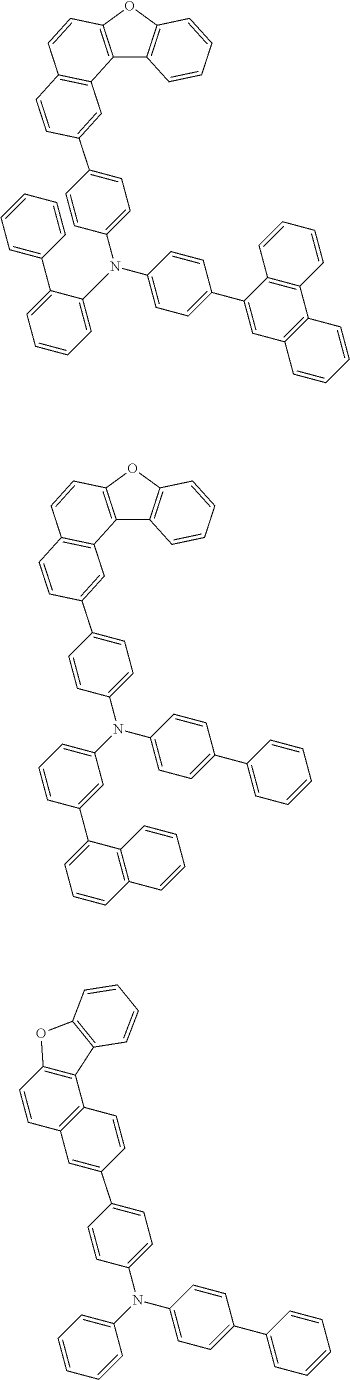 [SMILES: c1ccc(-c2ccccc2N(c2ccc(-c3ccc4ccc5oc6ccccc6c5c4c3)cc2)c2ccc(-c3cc4ccccc4c4ccccc34)cc2)cc1 ; c1ccc(-c2ccc(N(c3ccc(-c4ccc5ccc6oc7ccccc7c6c5c4)cc3)c3cccc(-c4cccc5ccccc45)c3)cc2)cc1 ; c1ccc(-c2ccc(N(c3ccccc3)c3ccc(-c4ccc5c(ccc6oc7ccccc7c65)c4)cc3)cc2)cc1] 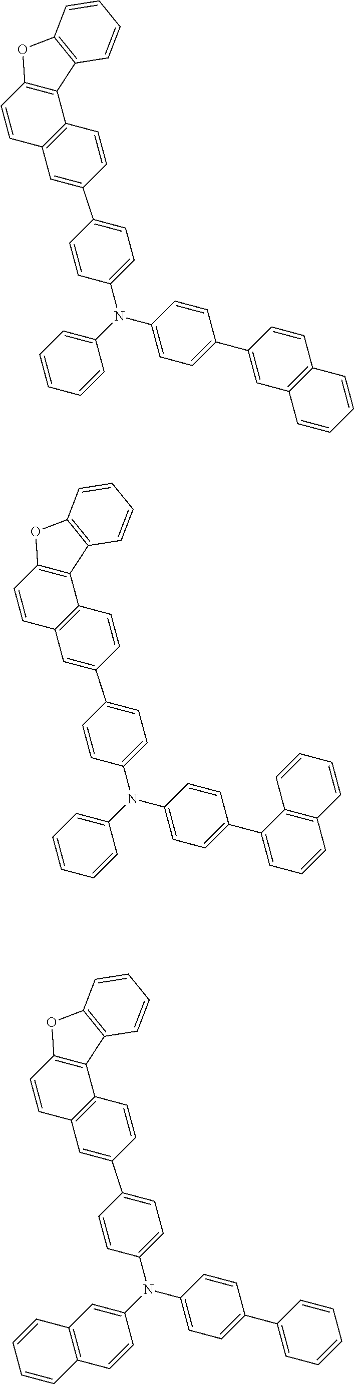 [SMILES: c1ccc(N(c2ccc(-c3ccc4ccccc4c3)cc2)c2ccc(-c3ccc4c(ccc5oc6ccccc6c54)c3)cc2)cc1 ; c1ccc(N(c2ccc(-c3ccc4c(ccc5oc6ccccc6c54)c3)cc2)c2ccc(-c3cccc4ccccc34)cc2)cc1 ; c1ccc(-c2ccc(N(c3ccc(-c4ccc5c(ccc6oc7ccccc7c65)c4)cc3)c3ccc4ccccc4c3)cc2)cc1] 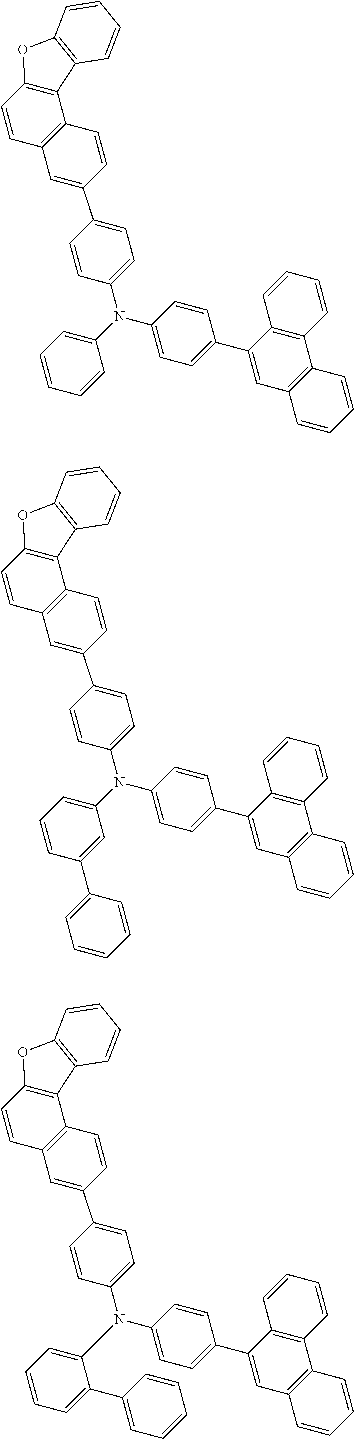 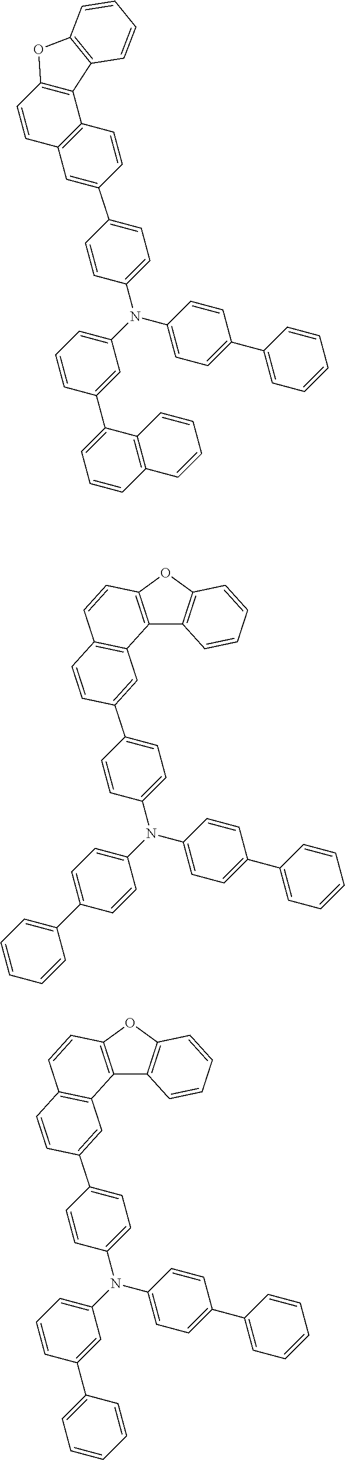 [SMILES: c1ccc(-c2ccc(N(c3ccc(-c4ccc5c(ccc6oc7ccccc7c65)c4)cc3)c3cccc(-c4cccc5ccccc45)c3)cc2)cc1 ; c1ccc(-c2ccc(N(c3ccc(-c4ccccc4)cc3)c3ccc(-c4ccc5ccc6oc7ccccc7c6c5c4)cc3)cc2)cc1 ; c1ccc(-c2ccc(N(c3ccc(-c4ccc5ccc6oc7ccccc7c6c5c4)cc3)c3cccc(-c4ccccc4)c3)cc2)cc1] 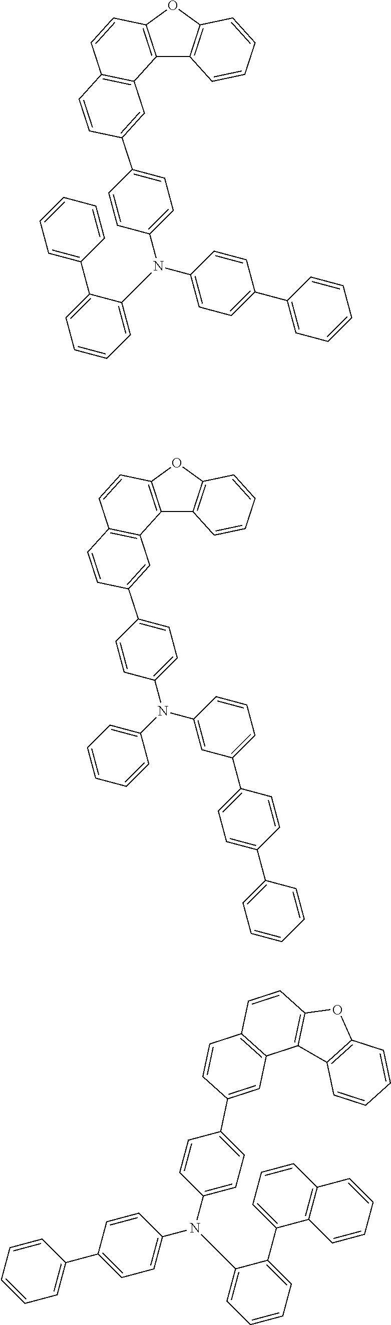 [SMILES: c1ccc(-c2ccc(N(c3ccc(-c4ccc5ccc6oc7ccccc7c6c5c4)cc3)c3ccccc3-c3ccccc3)cc2)cc1 ; c1ccc(-c2ccc(-c3cccc(N(c4ccccc4)c4ccc(-c5ccc6ccc7oc8ccccc8c7c6c5)cc4)c3)cc2)cc1 ; c1ccc(-c2ccc(N(c3ccc(-c4ccc5ccc6oc7ccccc7c6c5c4)cc3)c3ccccc3-c3cccc4ccccc34)cc2)cc1] 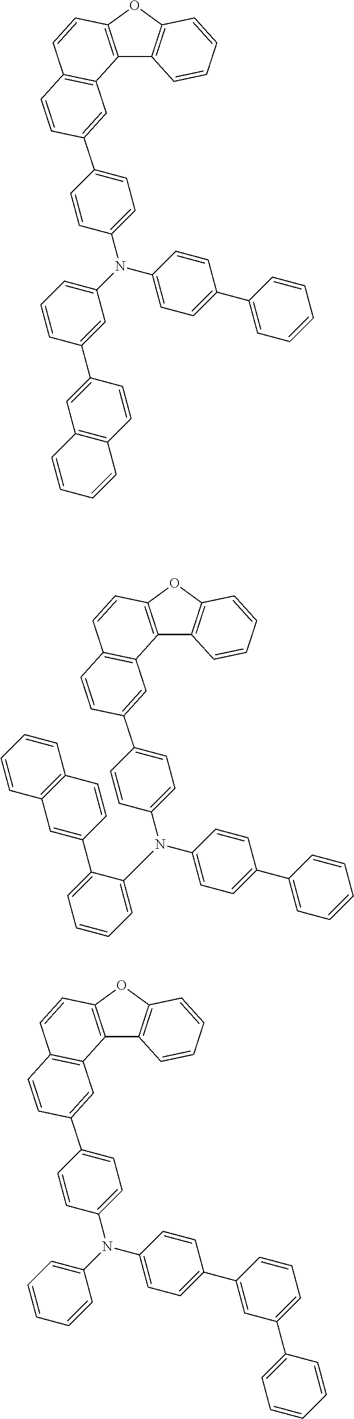 [SMILES: c1ccc(-c2ccc(N(c3ccc(-c4ccc5ccc6oc7ccccc7c6c5c4)cc3)c3cccc(-c4ccc5ccccc5c4)c3)cc2)cc1 ; c1ccc(-c2ccc(N(c3ccc(-c4ccc5ccc6oc7ccccc7c6c5c4)cc3)c3ccccc3-c3ccc4ccccc4c3)cc2)cc1 ; c1ccc(-c2cccc(-c3ccc(N(c4ccccc4)c4ccc(-c5ccc6ccc7oc8ccccc8c7c6c5)cc4)cc3)c2)cc1] 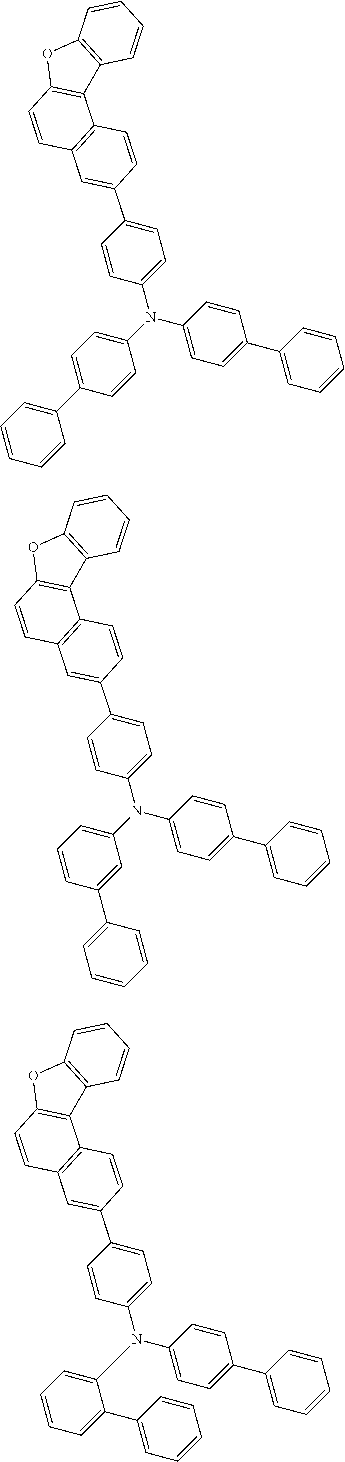 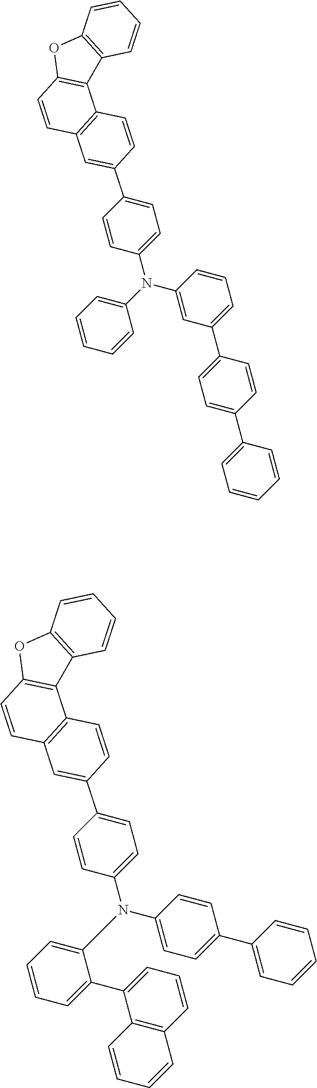 [SMILES: c1ccc(-c2ccc(-c3cccc(N(c4ccccc4)c4ccc(-c5ccc6c(ccc7oc8ccccc8c76)c5)cc4)c3)cc2)cc1 ; c1ccc(-c2ccc(N(c3ccc(-c4ccc5c(ccc6oc7ccccc7c65)c4)cc3)c3ccccc3-c3cccc4ccccc34)cc2)cc1] 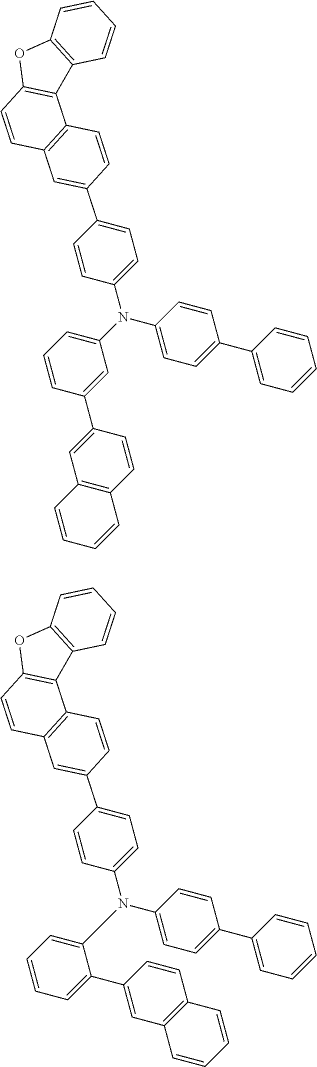 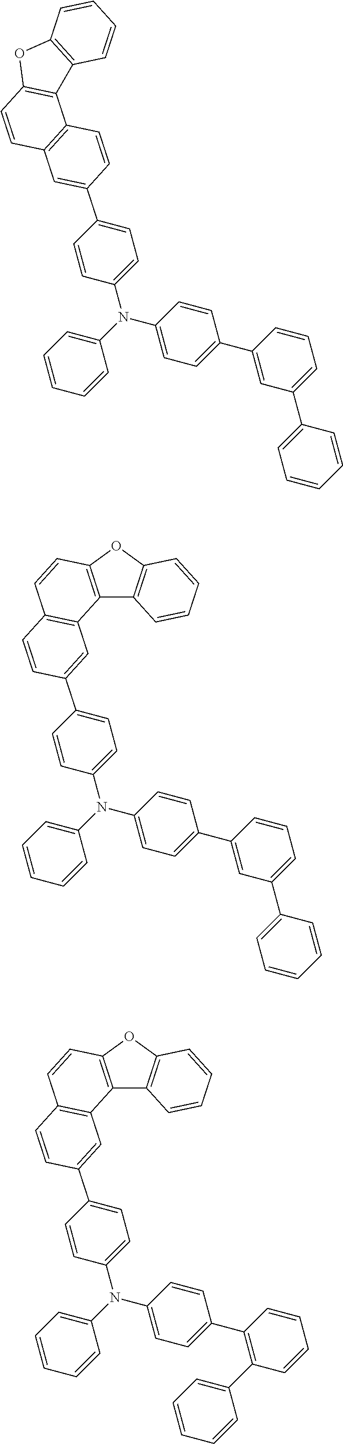 [SMILES: c1ccc(-c2cccc(-c3ccc(N(c4ccccc4)c4ccc(-c5ccc6c(ccc7oc8ccccc8c76)c5)cc4)cc3)c2)cc1 ; c1ccc(-c2cccc(-c3ccc(N(c4ccccc4)c4ccc(-c5ccc6ccc7oc8ccccc8c7c6c5)cc4)cc3)c2)cc1 ; c1ccc(-c2ccccc2-c2ccc(N(c3ccccc3)c3ccc(-c4ccc5ccc6oc7ccccc7c6c5c4)cc3)cc2)cc1] 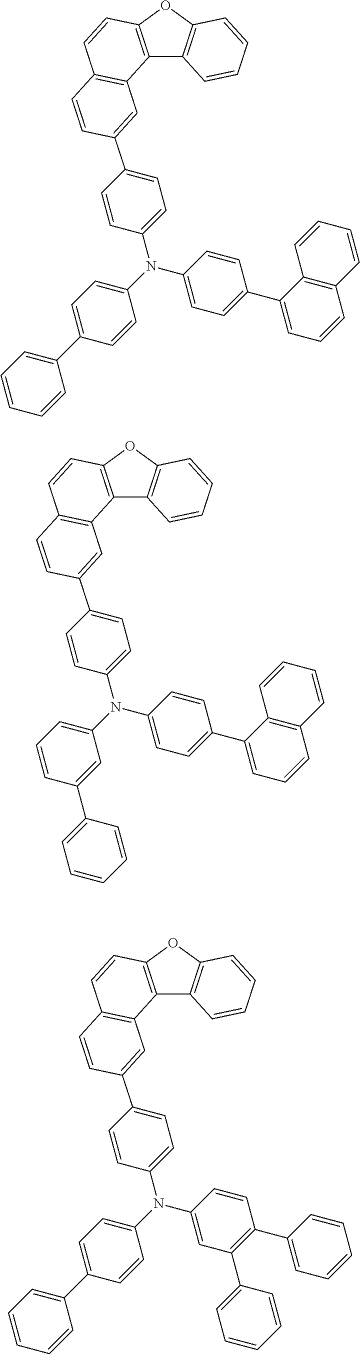 [SMILES: c1ccc(-c2ccc(N(c3ccc(-c4ccc5ccc6oc7ccccc7c6c5c4)cc3)c3ccc(-c4cccc5ccccc45)cc3)cc2)cc1 ; c1ccc(-c2cccc(N(c3ccc(-c4ccc5ccc6oc7ccccc7c6c5c4)cc3)c3ccc(-c4cccc5ccccc45)cc3)c2)cc1 ; c1ccc(-c2ccc(N(c3ccc(-c4ccc5ccc6oc7ccccc7c6c5c4)cc3)c3ccc(-c4ccccc4)c(-c4ccccc4)c3)cc2)cc1] 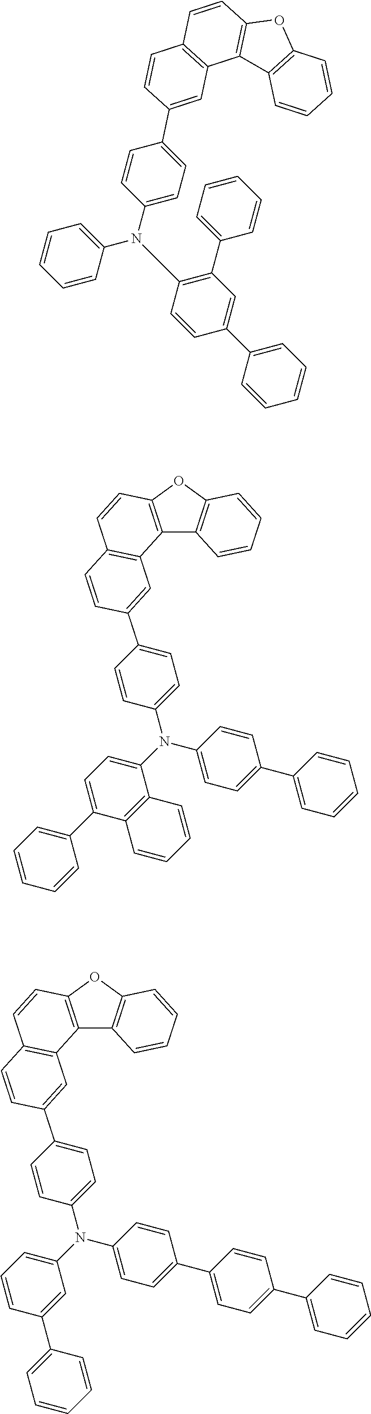 [SMILES: c1ccc(-c2ccc(N(c3ccccc3)c3ccc(-c4ccc5ccc6oc7ccccc7c6c5c4)cc3)c(-c3ccccc3)c2)cc1 ; c1ccc(-c2ccc(N(c3ccc(-c4ccc5ccc6oc7ccccc7c6c5c4)cc3)c3ccc(-c4ccccc4)c4ccccc34)cc2)cc1 ; c1ccc(-c2ccc(-c3ccc(N(c4ccc(-c5ccc6ccc7oc8ccccc8c7c6c5)cc4)c4cccc(-c5ccccc5)c4)cc3)cc2)cc1] 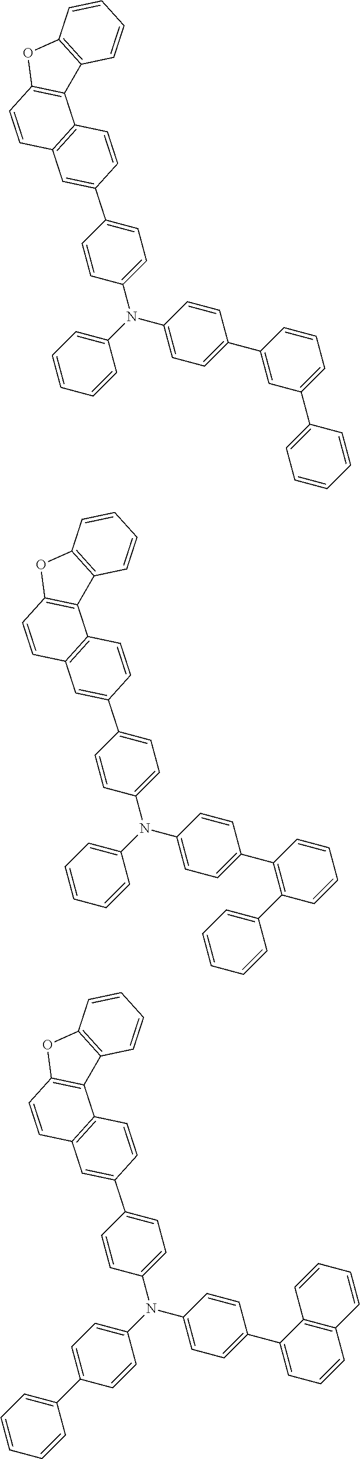 [SMILES: c1ccc(-c2cccc(-c3ccc(N(c4ccccc4)c4ccc(-c5ccc6c(ccc7oc8ccccc8c76)c5)cc4)cc3)c2)cc1 ; c1ccc(-c2ccccc2-c2ccc(N(c3ccccc3)c3ccc(-c4ccc5c(ccc6oc7ccccc7c65)c4)cc3)cc2)cc1 ; c1ccc(-c2ccc(N(c3ccc(-c4ccc5c(ccc6oc7ccccc7c65)c4)cc3)c3ccc(-c4cccc5ccccc45)cc3)cc2)cc1] 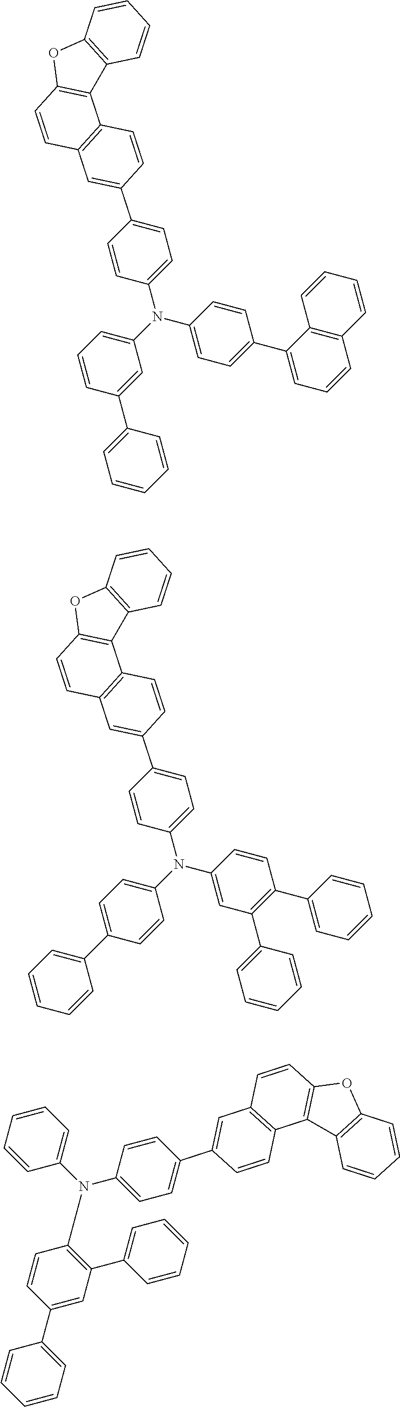 [SMILES: c1ccc(-c2cccc(N(c3ccc(-c4ccc5c(ccc6oc7ccccc7c65)c4)cc3)c3ccc(-c4cccc5ccccc45)cc3)c2)cc1 ; c1ccc(-c2ccc(N(c3ccc(-c4ccc5c(ccc6oc7ccccc7c65)c4)cc3)c3ccc(-c4ccccc4)c(-c4ccccc4)c3)cc2)cc1 ; c1ccc(-c2ccc(N(c3ccccc3)c3ccc(-c4ccc5c(ccc6oc7ccccc7c65)c4)cc3)c(-c3ccccc3)c2)cc1] 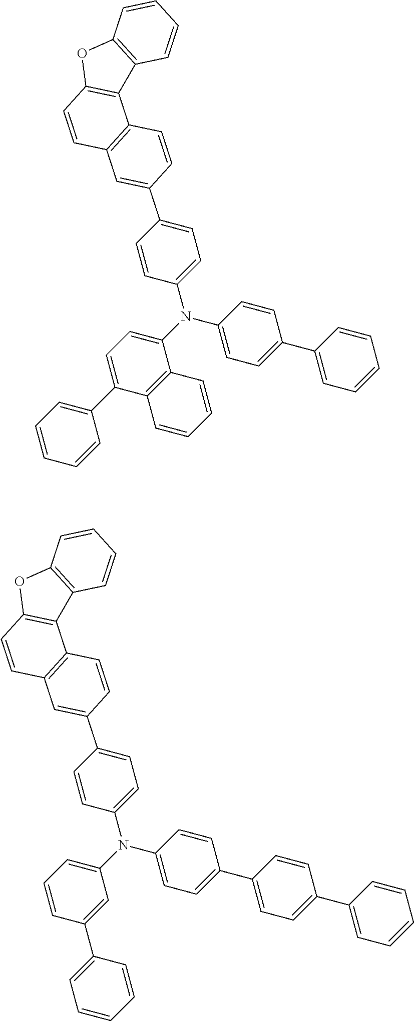 [SMILES: c1ccc(-c2ccc(N(c3ccc(-c4ccc5c(ccc6oc7ccccc7c65)c4)cc3)c3ccc(-c4ccccc4)c4ccccc34)cc2)cc1 ; c1ccc(-c2ccc(-c3ccc(N(c4ccc(-c5ccc6c(ccc7oc8ccccc8c76)c5)cc4)c4cccc(-c5ccccc5)c4)cc3)cc2)cc1] 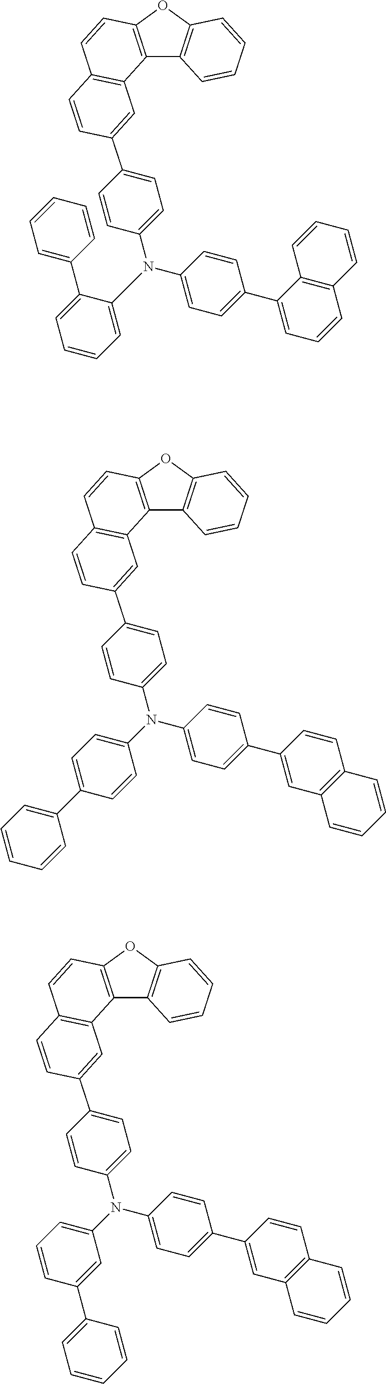 [SMILES: c1ccc(-c2ccccc2N(c2ccc(-c3ccc4ccc5oc6ccccc6c5c4c3)cc2)c2ccc(-c3cccc4ccccc34)cc2)cc1 ; c1ccc(-c2ccc(N(c3ccc(-c4ccc5ccccc5c4)cc3)c3ccc(-c4ccc5ccc6oc7ccccc7c6c5c4)cc3)cc2)cc1 ; c1ccc(-c2cccc(N(c3ccc(-c4ccc5ccccc5c4)cc3)c3ccc(-c4ccc5ccc6oc7ccccc7c6c5c4)cc3)c2)cc1] 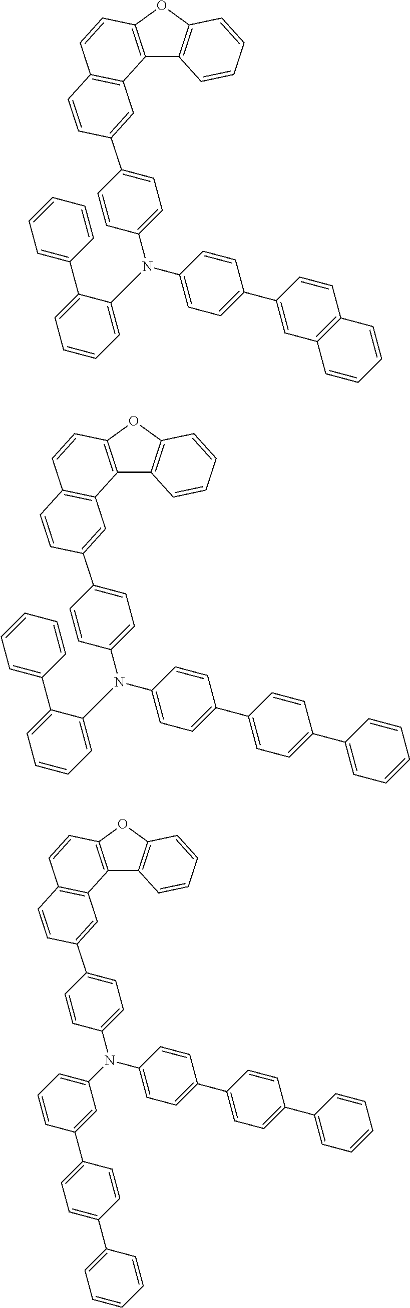 [SMILES: c1ccc(-c2ccccc2N(c2ccc(-c3ccc4ccccc4c3)cc2)c2ccc(-c3ccc4ccc5oc6ccccc6c5c4c3)cc2)cc1 ; c1ccc(-c2ccc(-c3ccc(N(c4ccc(-c5ccc6ccc7oc8ccccc8c7c6c5)cc4)c4ccccc4-c4ccccc4)cc3)cc2)cc1 ; c1ccc(-c2ccc(-c3ccc(N(c4ccc(-c5ccc6ccc7oc8ccccc8c7c6c5)cc4)c4cccc(-c5ccc(-c6ccccc6)cc5)c4)cc3)cc2)cc1] 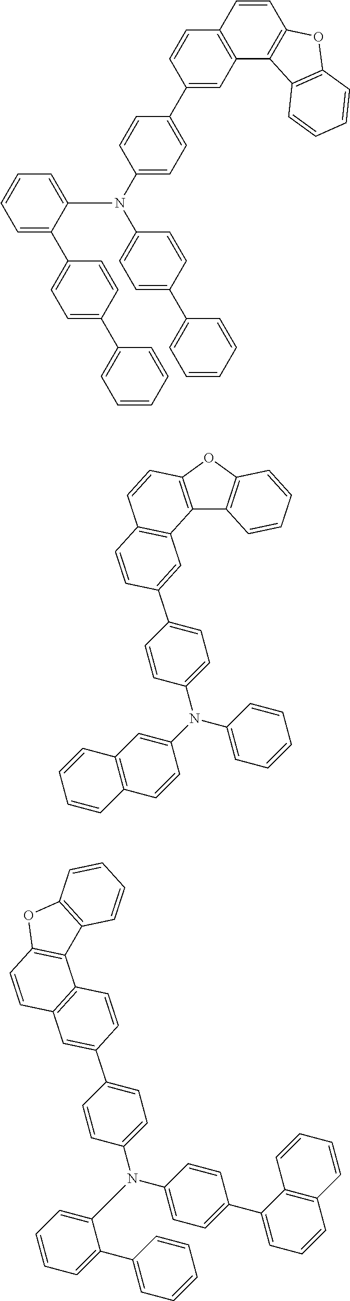 [SMILES: c1ccc(-c2ccc(-c3ccccc3N(c3ccc(-c4ccccc4)cc3)c3ccc(-c4ccc5ccc6oc7ccccc7c6c5c4)cc3)cc2)cc1 ; c1ccc(N(c2ccc(-c3ccc4ccc5oc6ccccc6c5c4c3)cc2)c2ccc3ccccc3c2)cc1 ; c1ccc(-c2ccccc2N(c2ccc(-c3ccc4c(ccc5oc6ccccc6c54)c3)cc2)c2ccc(-c3cccc4ccccc34)cc2)cc1] 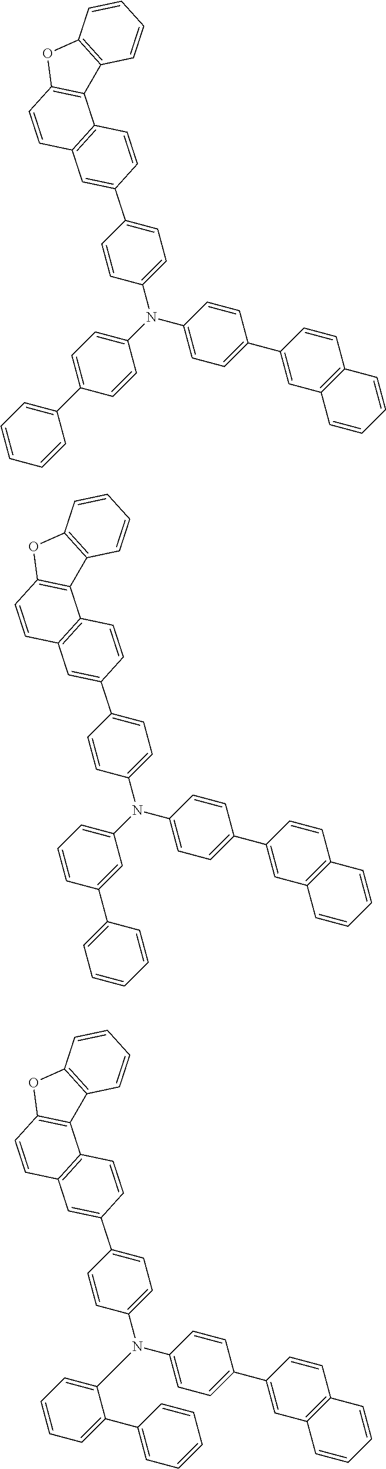 [SMILES: c1ccc(-c2ccc(N(c3ccc(-c4ccc5ccccc5c4)cc3)c3ccc(-c4ccc5c(ccc6oc7ccccc7c65)c4)cc3)cc2)cc1 ; c1ccc(-c2cccc(N(c3ccc(-c4ccc5ccccc5c4)cc3)c3ccc(-c4ccc5c(ccc6oc7ccccc7c65)c4)cc3)c2)cc1 ; c1ccc(-c2ccccc2N(c2ccc(-c3ccc4ccccc4c3)cc2)c2ccc(-c3ccc4c(ccc5oc6ccccc6c54)c3)cc2)cc1] 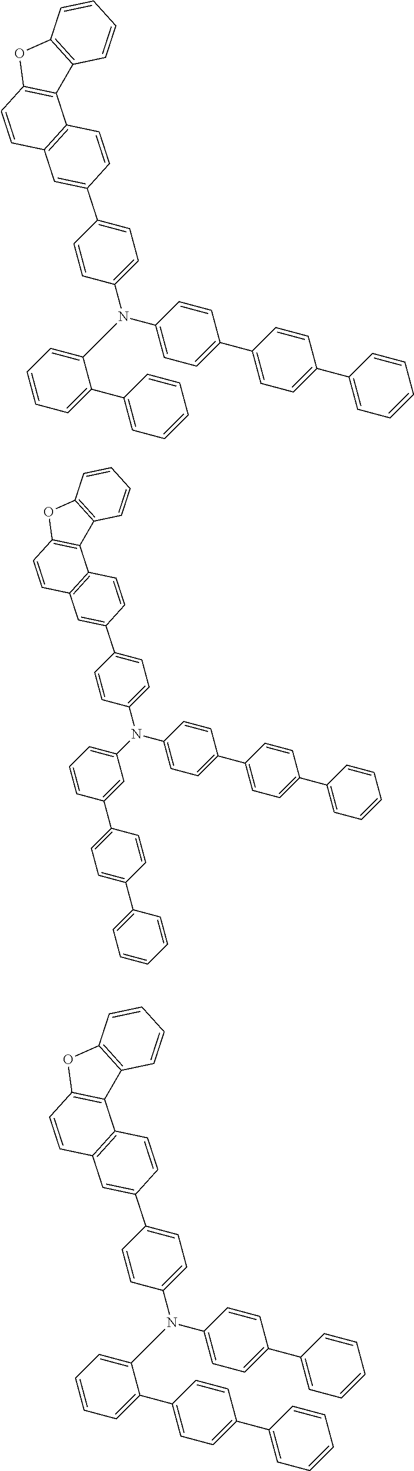 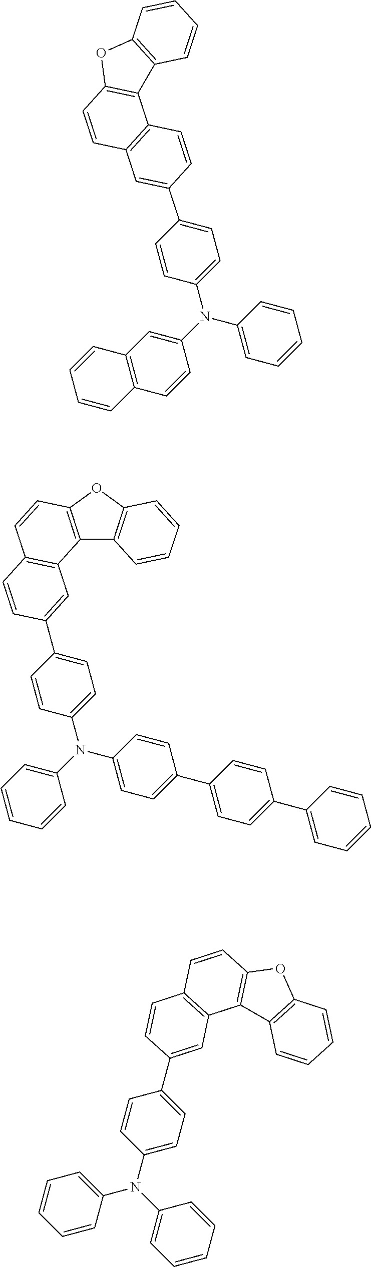 [SMILES: c1ccc(N(c2ccc(-c3ccc4c(ccc5oc6ccccc6c54)c3)cc2)c2ccc3ccccc3c2)cc1 ; c1ccc(-c2ccc(-c3ccc(N(c4ccccc4)c4ccc(-c5ccc6ccc7oc8ccccc8c7c6c5)cc4)cc3)cc2)cc1 ; c1ccc(N(c2ccccc2)c2ccc(-c3ccc4ccc5oc6ccccc6c5c4c3)cc2)cc1] 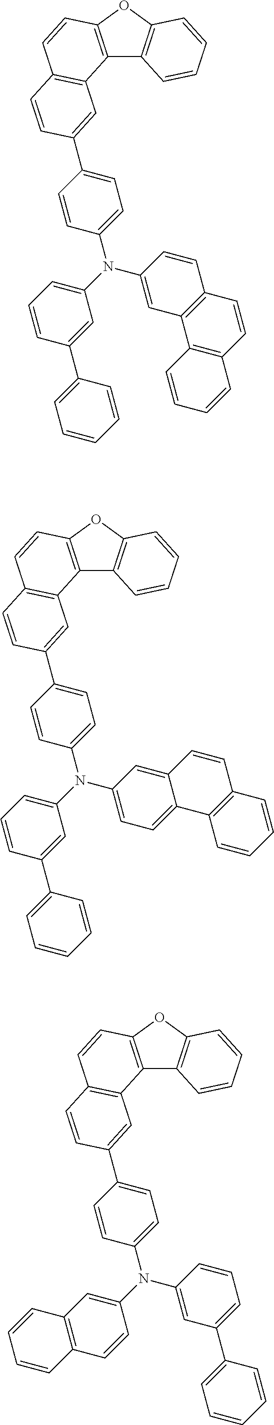 [SMILES: c1ccc(-c2cccc(N(c3ccc(-c4ccc5ccc6oc7ccccc7c6c5c4)cc3)c3ccc4ccc5ccccc5c4c3)c2)cc1 ; c1ccc(-c2cccc(N(c3ccc(-c4ccc5ccc6oc7ccccc7c6c5c4)cc3)c3ccc4c(ccc5ccccc54)c3)c2)cc1 ; c1ccc(-c2cccc(N(c3ccc(-c4ccc5ccc6oc7ccccc7c6c5c4)cc3)c3ccc4ccccc4c3)c2)cc1] 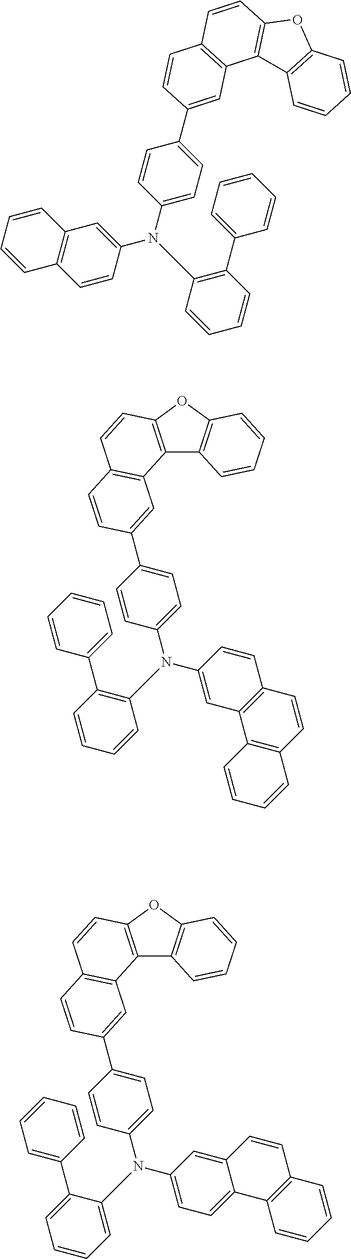 [SMILES: c1ccc(-c2ccccc2N(c2ccc(-c3ccc4ccc5oc6ccccc6c5c4c3)cc2)c2ccc3ccccc3c2)cc1 ; c1ccc(-c2ccccc2N(c2ccc(-c3ccc4ccc5oc6ccccc6c5c4c3)cc2)c2ccc3ccc4ccccc4c3c2)cc1 ; c1ccc(-c2ccccc2N(c2ccc(-c3ccc4ccc5oc6ccccc6c5c4c3)cc2)c2ccc3c(ccc4ccccc43)c2)cc1] 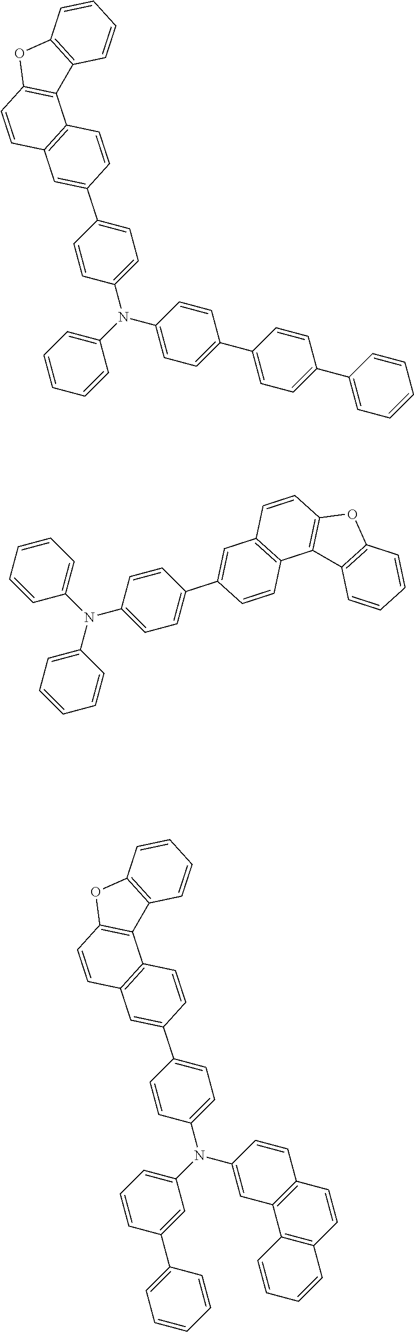 [SMILES: c1ccc(-c2ccc(-c3ccc(N(c4ccccc4)c4ccc(-c5ccc6c(ccc7oc8ccccc8c76)c5)cc4)cc3)cc2)cc1 ; c1ccc(N(c2ccccc2)c2ccc(-c3ccc4c(ccc5oc6ccccc6c54)c3)cc2)cc1 ; c1ccc(-c2cccc(N(c3ccc(-c4ccc5c(ccc6oc7ccccc7c65)c4)cc3)c3ccc4ccc5ccccc5c4c3)c2)cc1] 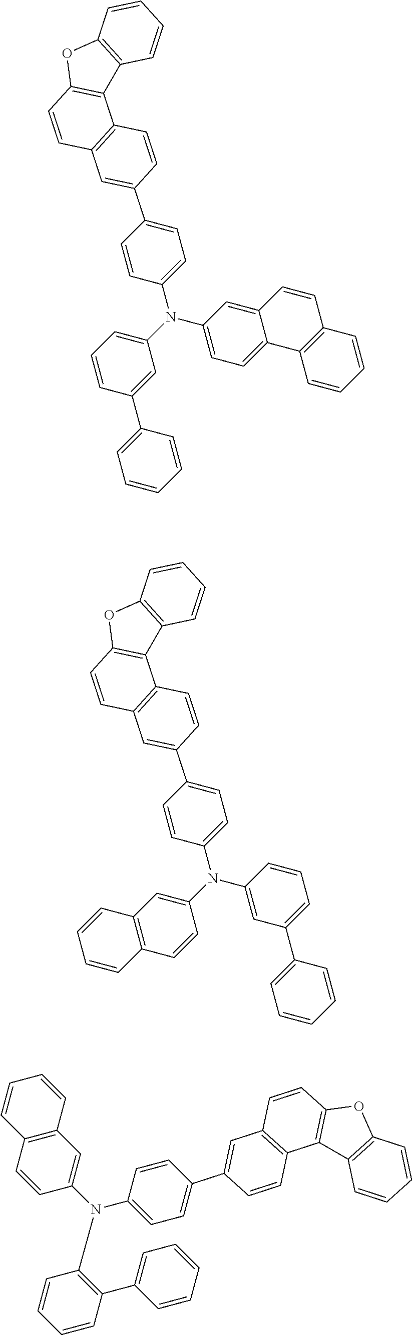 [SMILES: c1ccc(-c2cccc(N(c3ccc(-c4ccc5c(ccc6oc7ccccc7c65)c4)cc3)c3ccc4c(ccc5ccccc54)c3)c2)cc1 ; c1ccc(-c2cccc(N(c3ccc(-c4ccc5c(ccc6oc7ccccc7c65)c4)cc3)c3ccc4ccccc4c3)c2)cc1 ; c1ccc(-c2ccccc2N(c2ccc(-c3ccc4c(ccc5oc6ccccc6c54)c3)cc2)c2ccc3ccccc3c2)cc1] 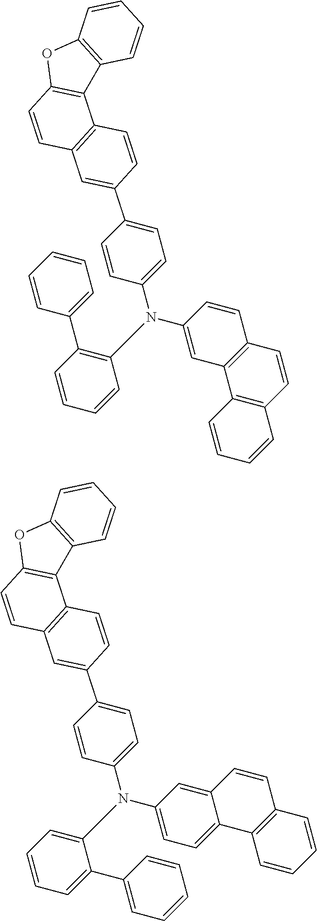 [SMILES: c1ccc(-c2ccccc2N(c2ccc(-c3ccc4c(ccc5oc6ccccc6c54)c3)cc2)c2ccc3ccc4ccccc4c3c2)cc1 ; c1ccc(-c2ccccc2N(c2ccc(-c3ccc4c(ccc5oc6ccccc6c54)c3)cc2)c2ccc3c(ccc4ccccc43)c2)cc1] 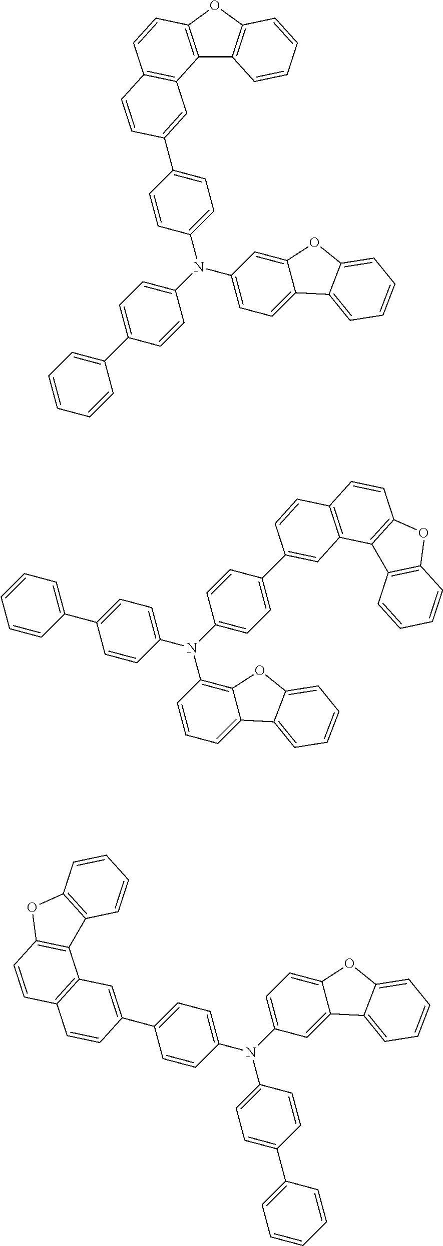 [SMILES: c1ccc(-c2ccc(N(c3ccc(-c4ccc5ccc6oc7ccccc7c6c5c4)cc3)c3ccc4c(c3)oc3ccccc34)cc2)cc1 ; c1ccc(-c2ccc(N(c3ccc(-c4ccc5ccc6oc7ccccc7c6c5c4)cc3)c3cccc4c3oc3ccccc34)cc2)cc1 ; c1ccc(-c2ccc(N(c3ccc(-c4ccc5ccc6oc7ccccc7c6c5c4)cc3)c3ccc4oc5ccccc5c4c3)cc2)cc1] 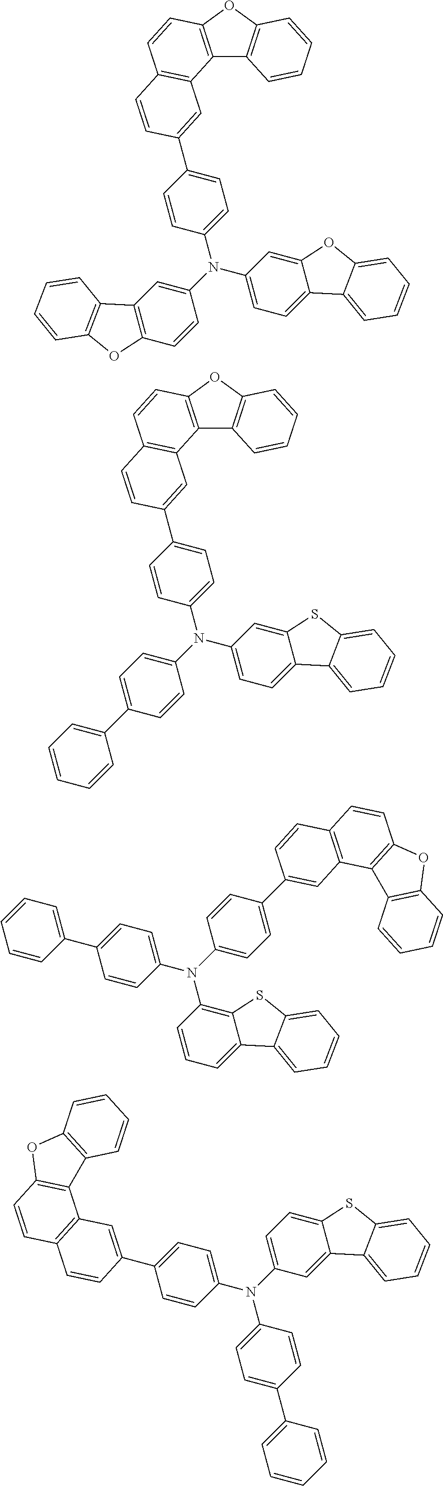 [SMILES: c1ccc2c(c1)oc1cc(N(c3ccc(-c4ccc5ccc6oc7ccccc7c6c5c4)cc3)c3ccc4oc5ccccc5c4c3)ccc12 ; c1ccc(-c2ccc(N(c3ccc(-c4ccc5ccc6oc7ccccc7c6c5c4)cc3)c3ccc4c(c3)sc3ccccc34)cc2)cc1 ; c1ccc(-c2ccc(N(c3ccc(-c4ccc5ccc6oc7ccccc7c6c5c4)cc3)c3cccc4c3sc3ccccc34)cc2)cc1 ; c1ccc(-c2ccc(N(c3ccc(-c4ccc5ccc6oc7ccccc7c6c5c4)cc3)c3ccc4sc5ccccc5c4c3)cc2)cc1] 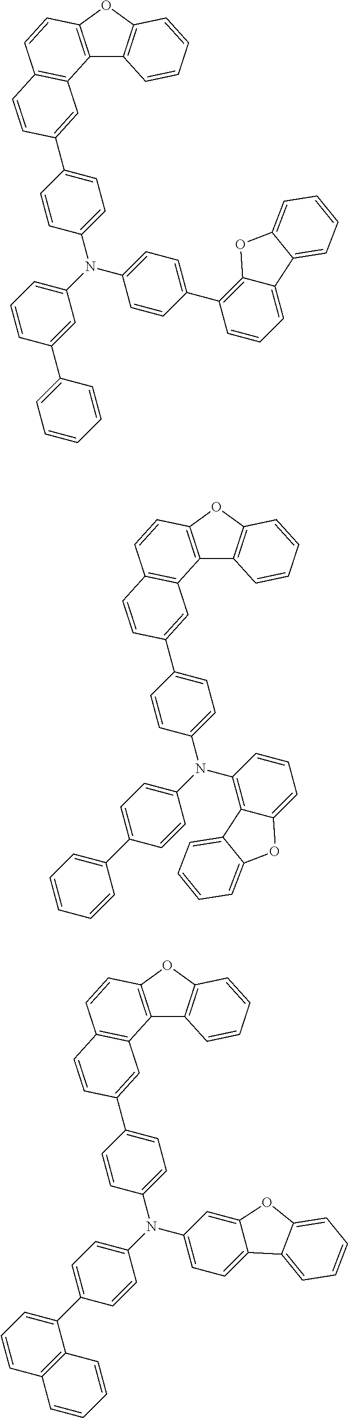 [SMILES: c1ccc(-c2cccc(N(c3ccc(-c4ccc5ccc6oc7ccccc7c6c5c4)cc3)c3ccc(-c4cccc5c4oc4ccccc45)cc3)c2)cc1 ; c1ccc(-c2ccc(N(c3ccc(-c4ccc5ccc6oc7ccccc7c6c5c4)cc3)c3cccc4oc5ccccc5c34)cc2)cc1 ; c1ccc2c(-c3ccc(N(c4ccc(-c5ccc6ccc7oc8ccccc8c7c6c5)cc4)c4ccc5c(c4)oc4ccccc45)cc3)cccc2c1] 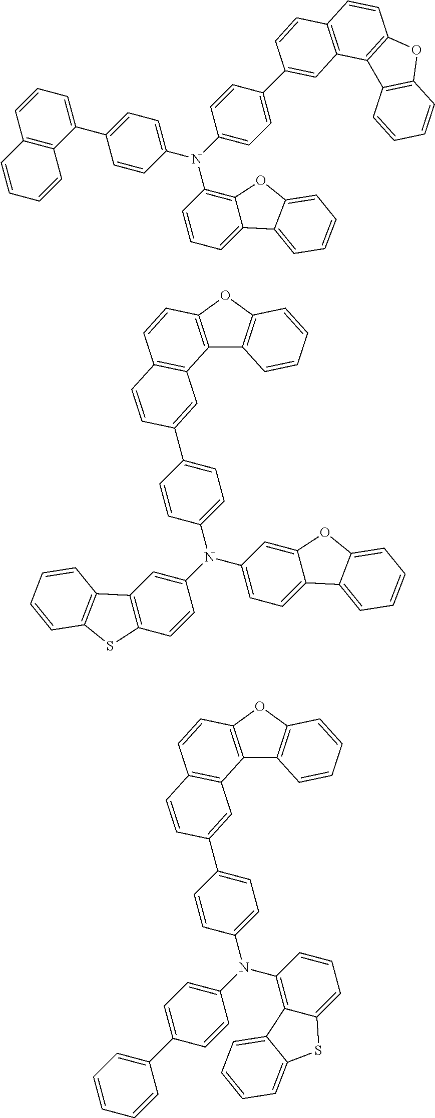 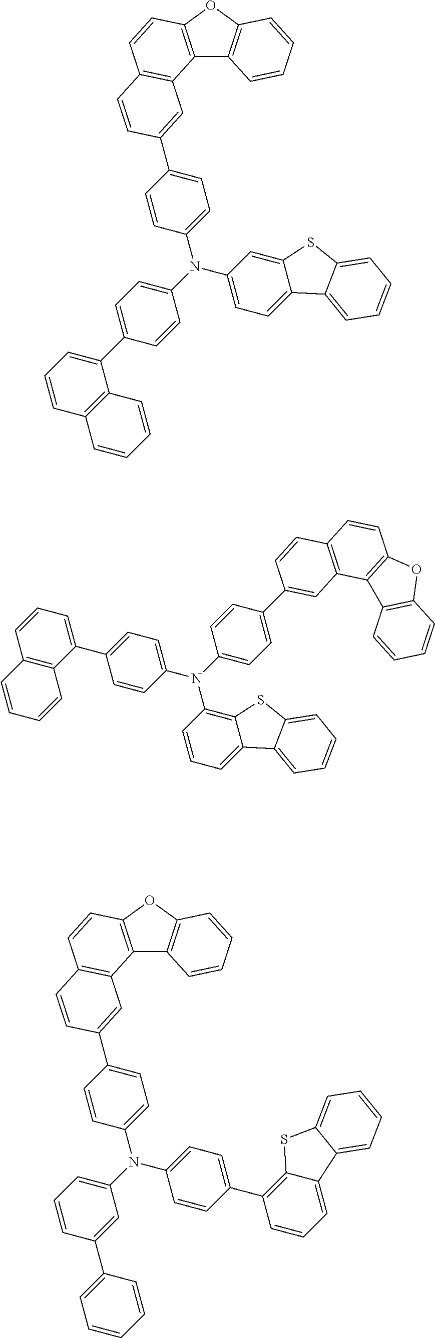 [SMILES: c1ccc2c(-c3ccc(N(c4ccc(-c5ccc6ccc7oc8ccccc8c7c6c5)cc4)c4ccc5c(c4)sc4ccccc45)cc3)cccc2c1 ; c1ccc2c(-c3ccc(N(c4ccc(-c5ccc6ccc7oc8ccccc8c7c6c5)cc4)c4cccc5c4sc4ccccc45)cc3)cccc2c1 ; c1ccc(-c2cccc(N(c3ccc(-c4ccc5ccc6oc7ccccc7c6c5c4)cc3)c3ccc(-c4cccc5c4sc4ccccc45)cc3)c2)cc1] 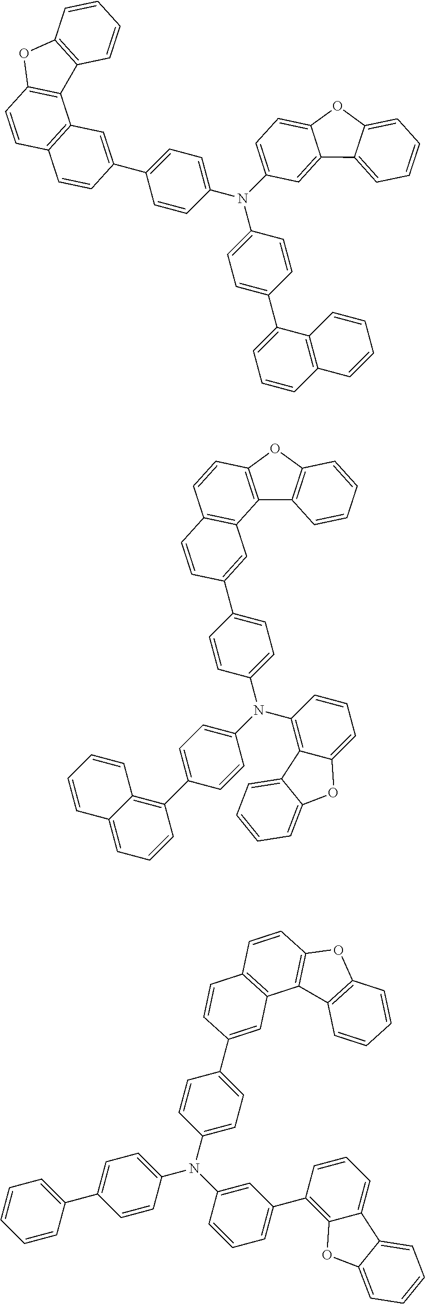 [SMILES: c1ccc2c(-c3ccc(N(c4ccc(-c5ccc6ccc7oc8ccccc8c7c6c5)cc4)c4ccc5oc6ccccc6c5c4)cc3)cccc2c1 ; c1ccc2c(-c3ccc(N(c4ccc(-c5ccc6ccc7oc8ccccc8c7c6c5)cc4)c4cccc5oc6ccccc6c45)cc3)cccc2c1 ; c1ccc(-c2ccc(N(c3ccc(-c4ccc5ccc6oc7ccccc7c6c5c4)cc3)c3cccc(-c4cccc5c4oc4ccccc45)c3)cc2)cc1] 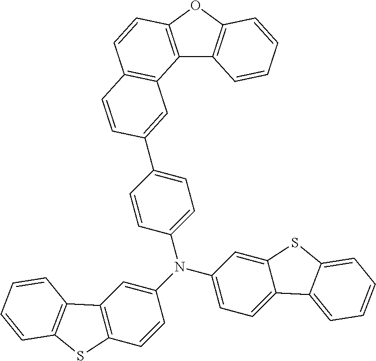 [SMILES: c1ccc2c(c1)oc1ccc3ccc(-c4ccc(N(c5ccc6c(c5)sc5ccccc56)c5ccc6sc7ccccc7c6c5)cc4)cc3c12] 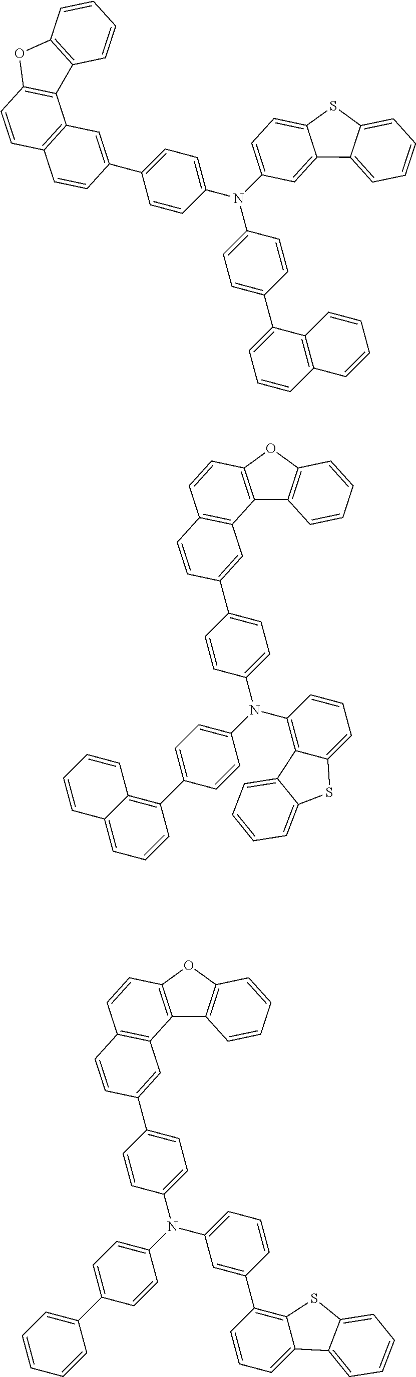 [SMILES: c1ccc2c(-c3ccc(N(c4ccc(-c5ccc6ccc7oc8ccccc8c7c6c5)cc4)c4ccc5sc6ccccc6c5c4)cc3)cccc2c1 ; c1ccc2c(-c3ccc(N(c4ccc(-c5ccc6ccc7oc8ccccc8c7c6c5)cc4)c4cccc5sc6ccccc6c45)cc3)cccc2c1 ; c1ccc(-c2ccc(N(c3ccc(-c4ccc5ccc6oc7ccccc7c6c5c4)cc3)c3cccc(-c4cccc5c4sc4ccccc45)c3)cc2)cc1] 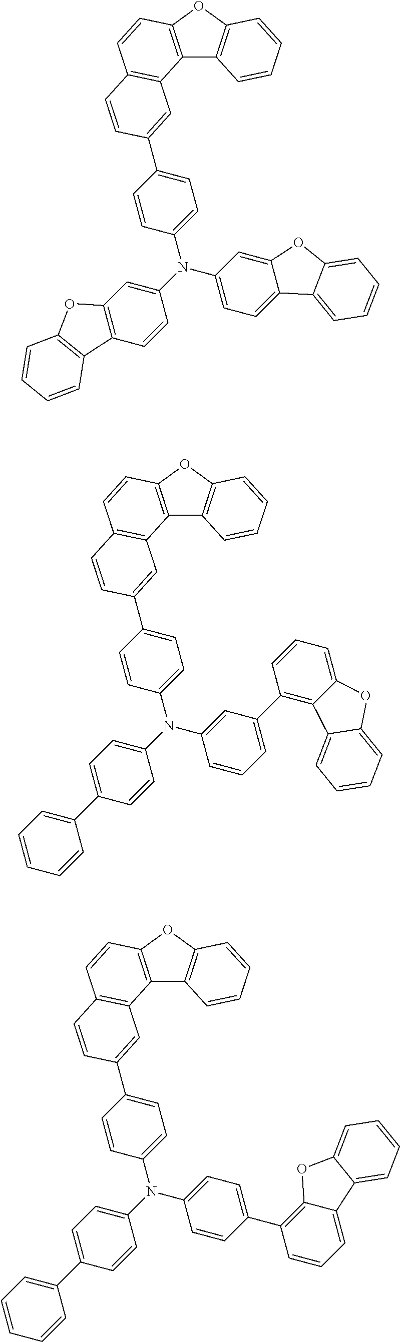 [SMILES: c1ccc2c(c1)oc1cc(N(c3ccc(-c4ccc5ccc6oc7ccccc7c6c5c4)cc3)c3ccc4c(c3)oc3ccccc34)ccc12 ; c1ccc(-c2ccc(N(c3ccc(-c4ccc5ccc6oc7ccccc7c6c5c4)cc3)c3cccc(-c4cccc5oc6ccccc6c45)c3)cc2)cc1 ; c1ccc(-c2ccc(N(c3ccc(-c4ccc5ccc6oc7ccccc7c6c5c4)cc3)c3ccc(-c4cccc5c4oc4ccccc45)cc3)cc2)cc1] 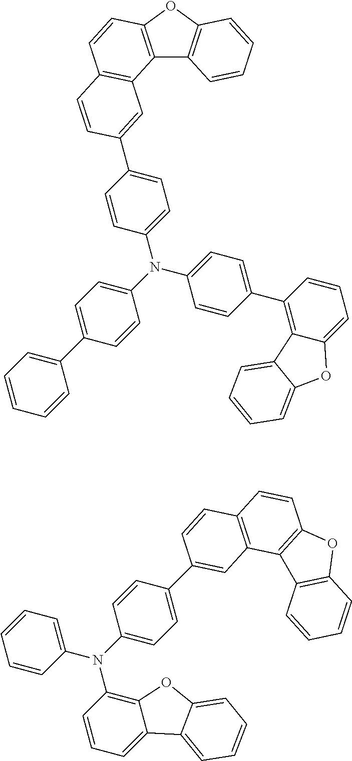 [SMILES: c1ccc(-c2ccc(N(c3ccc(-c4ccc5ccc6oc7ccccc7c6c5c4)cc3)c3ccc(-c4cccc5oc6ccccc6c45)cc3)cc2)cc1 ; c1ccc(N(c2ccc(-c3ccc4ccc5oc6ccccc6c5c4c3)cc2)c2cccc3c2oc2ccccc23)cc1] 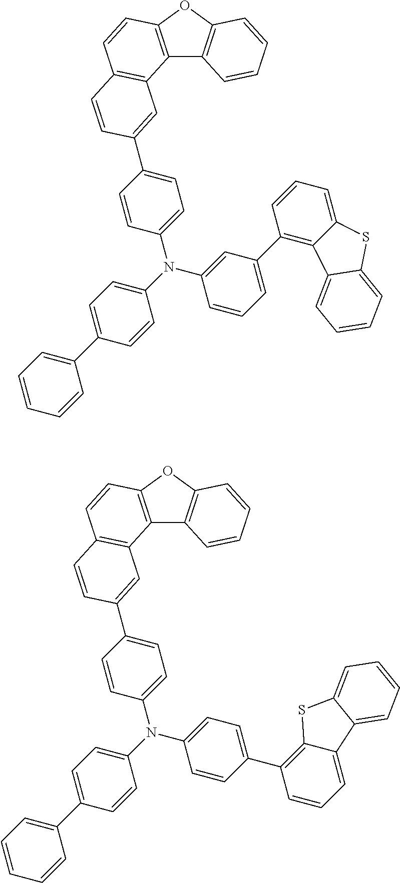 [SMILES: c1ccc(-c2ccc(N(c3ccc(-c4ccc5ccc6oc7ccccc7c6c5c4)cc3)c3cccc(-c4cccc5sc6ccccc6c45)c3)cc2)cc1 ; c1ccc(-c2ccc(N(c3ccc(-c4ccc5ccc6oc7ccccc7c6c5c4)cc3)c3ccc(-c4cccc5c4sc4ccccc45)cc3)cc2)cc1] 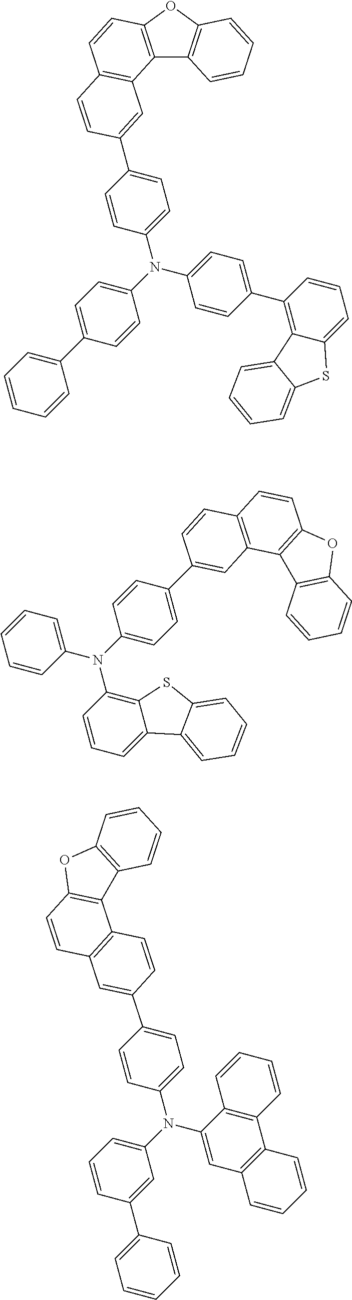 [SMILES: c1ccc(-c2ccc(N(c3ccc(-c4ccc5ccc6oc7ccccc7c6c5c4)cc3)c3ccc(-c4cccc5sc6ccccc6c45)cc3)cc2)cc1 ; c1ccc(N(c2ccc(-c3ccc4ccc5oc6ccccc6c5c4c3)cc2)c2cccc3c2sc2ccccc23)cc1 ; c1ccc(-c2cccc(N(c3ccc(-c4ccc5c(ccc6oc7ccccc7c65)c4)cc3)c3cc4ccccc4c4ccccc34)c2)cc1] 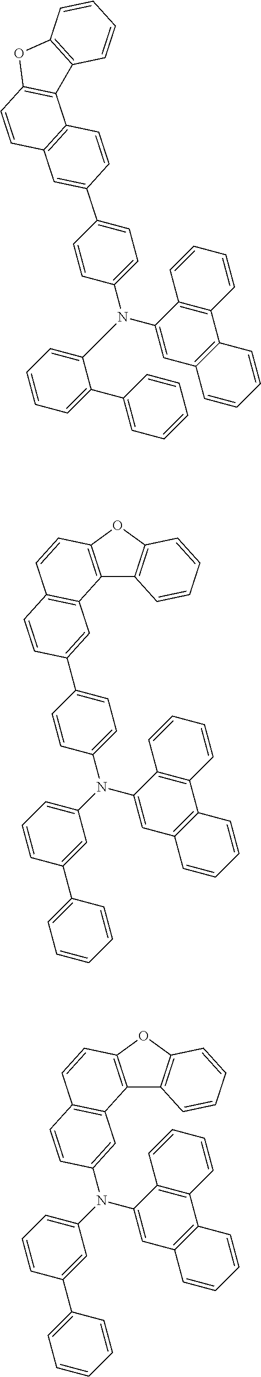 [SMILES: c1ccc(-c2ccccc2N(c2ccc(-c3ccc4c(ccc5oc6ccccc6c54)c3)cc2)c2cc3ccccc3c3ccccc23)cc1 ; c1ccc(-c2cccc(N(c3ccc(-c4ccc5ccc6oc7ccccc7c6c5c4)cc3)c3cc4ccccc4c4ccccc34)c2)cc1 ; c1ccc(-c2cccc(N(c3ccc4ccc5oc6ccccc6c5c4c3)c3cc4ccccc4c4ccccc34)c2)cc1] 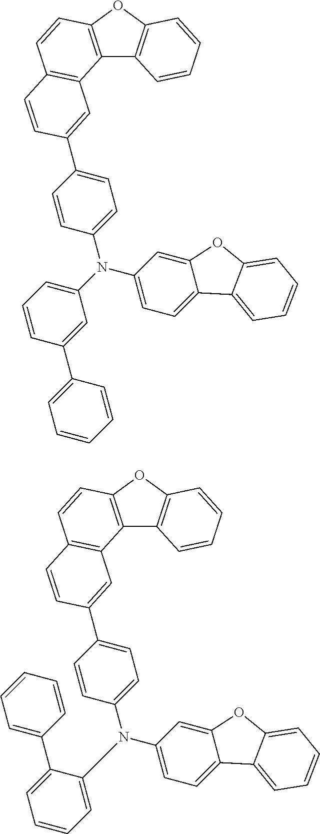 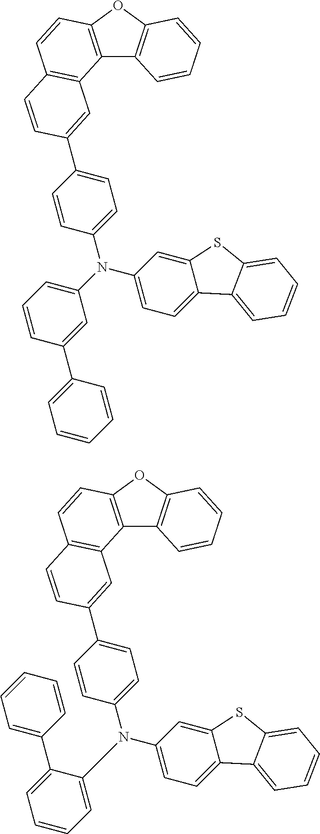 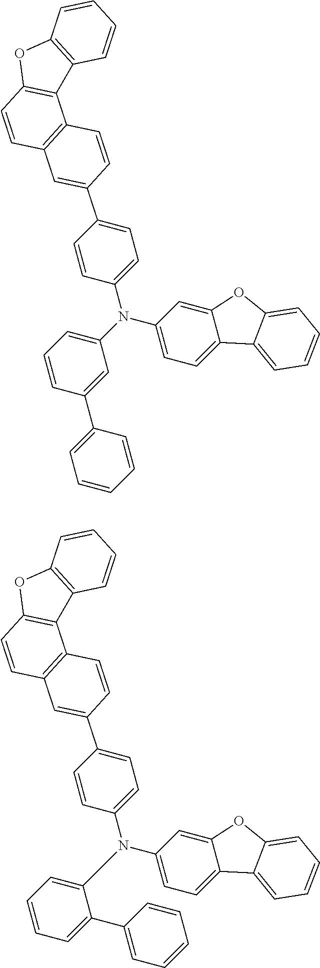 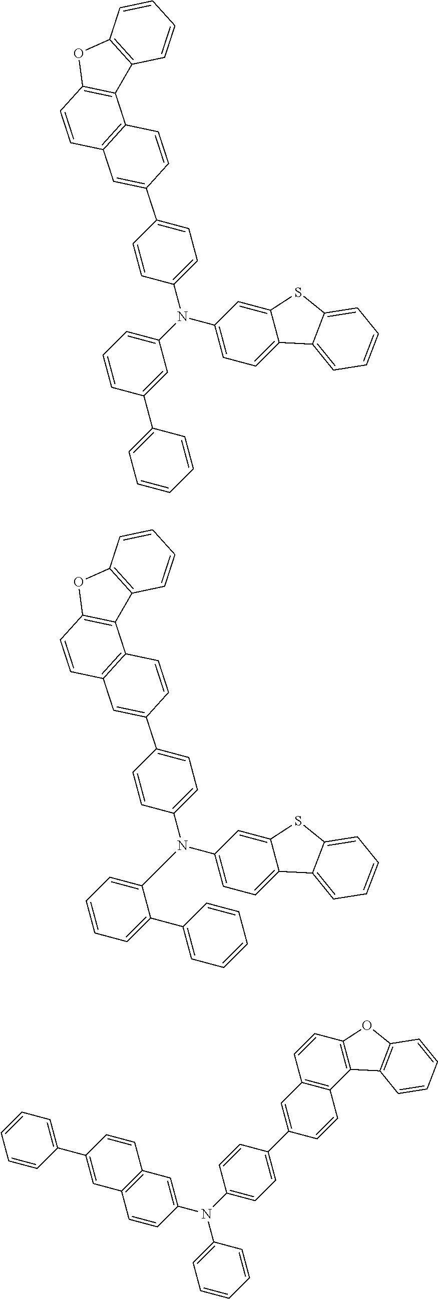 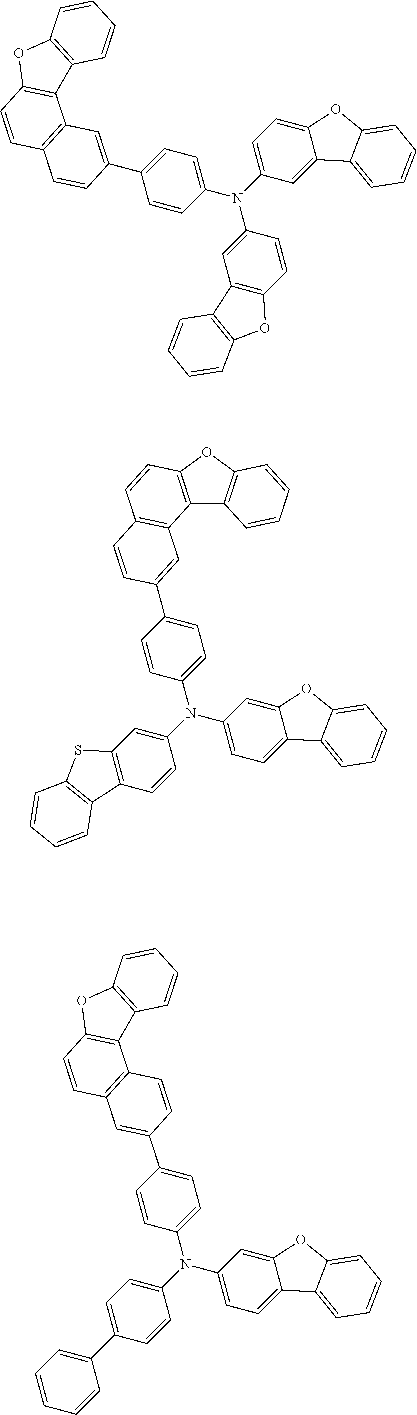 [SMILES: c1ccc2c(c1)oc1ccc(N(c3ccc(-c4ccc5ccc6oc7ccccc7c6c5c4)cc3)c3ccc4oc5ccccc5c4c3)cc12 ; c1ccc2c(c1)oc1cc(N(c3ccc(-c4ccc5ccc6oc7ccccc7c6c5c4)cc3)c3ccc4c(c3)sc3ccccc34)ccc12 ; c1ccc(-c2ccc(N(c3ccc(-c4ccc5c(ccc6oc7ccccc7c65)c4)cc3)c3ccc4c(c3)oc3ccccc34)cc2)cc1] 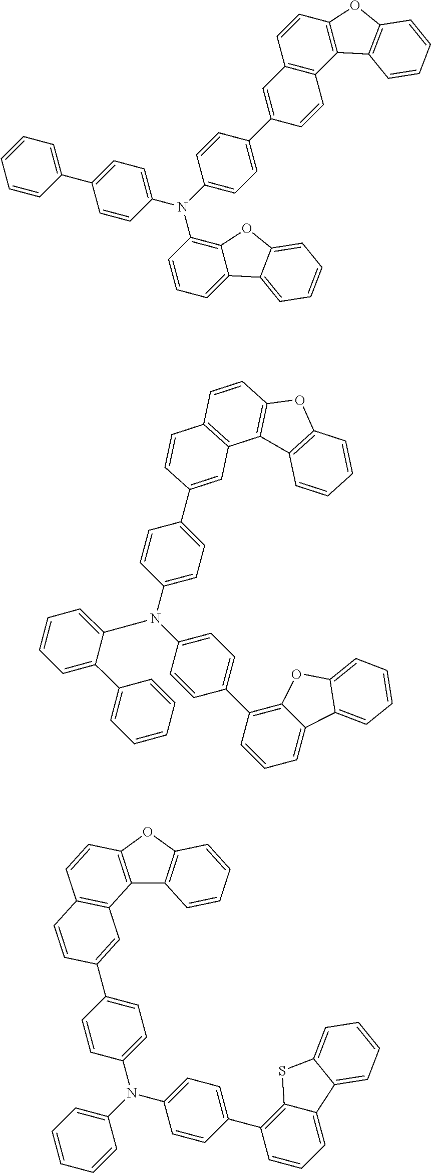 [SMILES: c1ccc(-c2ccc(N(c3ccc(-c4ccc5c(ccc6oc7ccccc7c65)c4)cc3)c3cccc4c3oc3ccccc34)cc2)cc1 ; c1ccc(-c2ccccc2N(c2ccc(-c3ccc4ccc5oc6ccccc6c5c4c3)cc2)c2ccc(-c3cccc4c3oc3ccccc34)cc2)cc1 ; c1ccc(N(c2ccc(-c3ccc4ccc5oc6ccccc6c5c4c3)cc2)c2ccc(-c3cccc4c3sc3ccccc34)cc2)cc1] 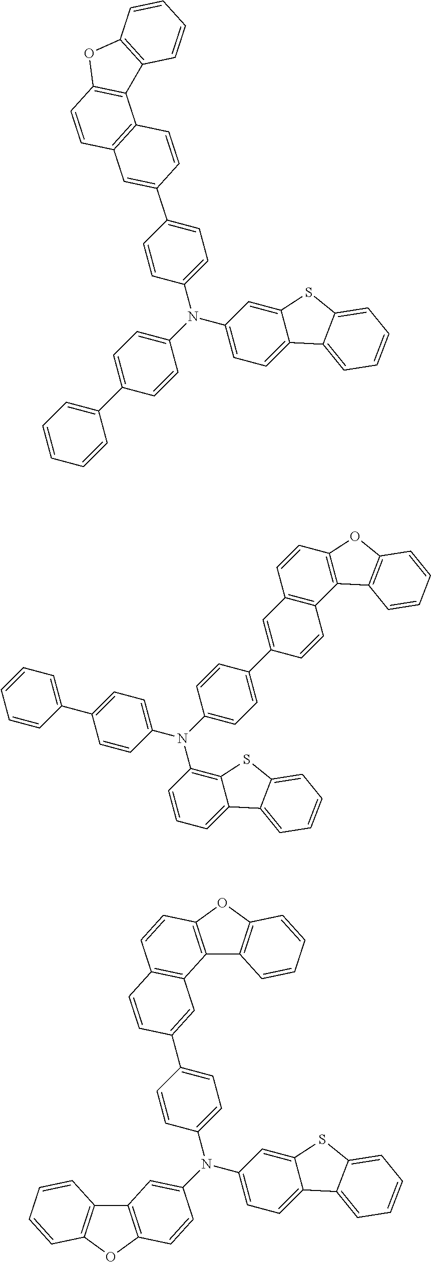 [SMILES: c1ccc(-c2ccc(N(c3ccc(-c4ccc5c(ccc6oc7ccccc7c65)c4)cc3)c3ccc4c(c3)sc3ccccc34)cc2)cc1 ; c1ccc(-c2ccc(N(c3ccc(-c4ccc5c(ccc6oc7ccccc7c65)c4)cc3)c3cccc4c3sc3ccccc34)cc2)cc1 ; c1ccc2c(c1)oc1ccc(N(c3ccc(-c4ccc5ccc6oc7ccccc7c6c5c4)cc3)c3ccc4c(c3)sc3ccccc34)cc12] 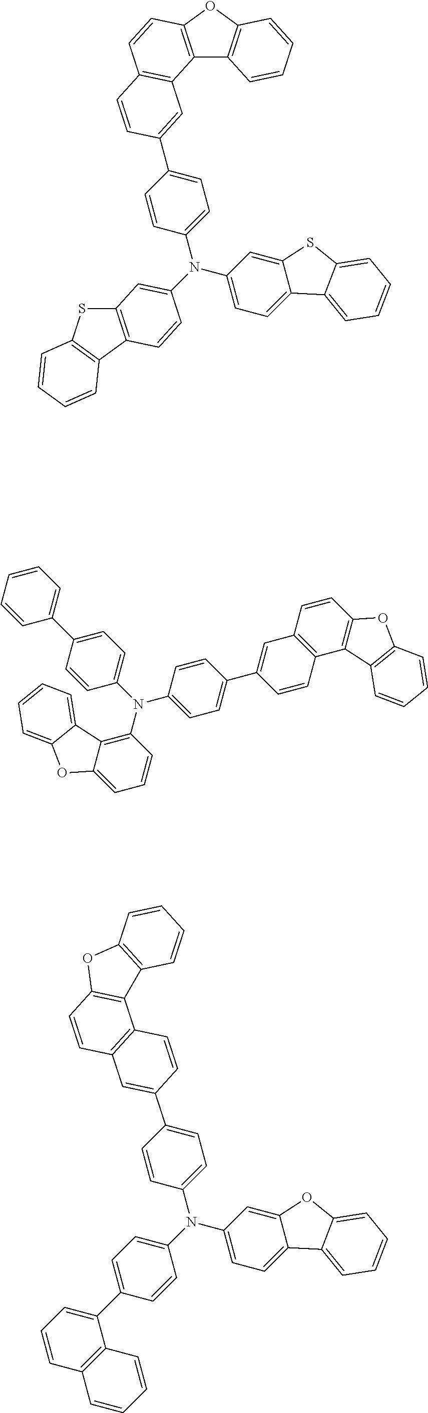 [SMILES: c1ccc2c(c1)oc1ccc3ccc(-c4ccc(N(c5ccc6c(c5)sc5ccccc56)c5ccc6c(c5)sc5ccccc56)cc4)cc3c12 ; c1ccc(-c2ccc(N(c3ccc(-c4ccc5c(ccc6oc7ccccc7c65)c4)cc3)c3cccc4oc5ccccc5c34)cc2)cc1 ; c1ccc2c(-c3ccc(N(c4ccc(-c5ccc6c(ccc7oc8ccccc8c76)c5)cc4)c4ccc5c(c4)oc4ccccc45)cc3)cccc2c1] 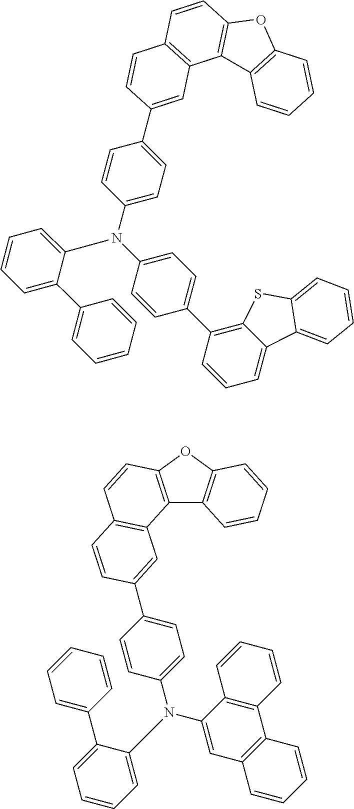 [SMILES: c1ccc(-c2ccccc2N(c2ccc(-c3ccc4ccc5oc6ccccc6c5c4c3)cc2)c2ccc(-c3cccc4c3sc3ccccc34)cc2)cc1 ; c1ccc(-c2ccccc2N(c2ccc(-c3ccc4ccc5oc6ccccc6c5c4c3)cc2)c2cc3ccccc3c3ccccc23)cc1] 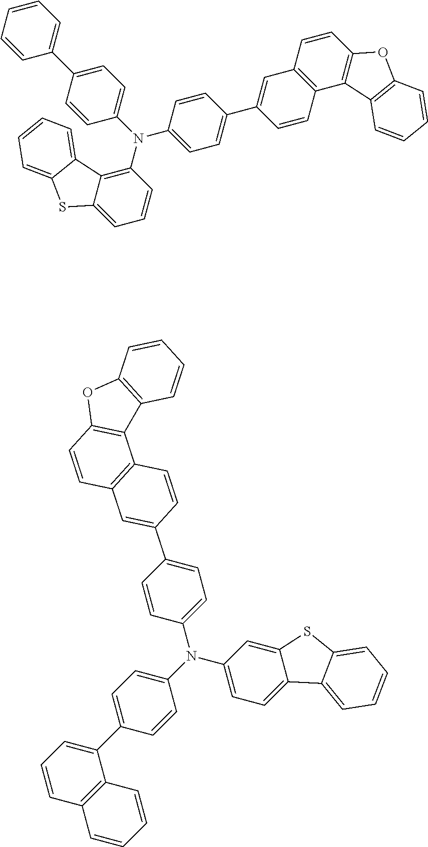 [SMILES: c1ccc(-c2ccc(N(c3ccc(-c4ccc5c(ccc6oc7ccccc7c65)c4)cc3)c3cccc4sc5ccccc5c34)cc2)cc1 ; c1ccc2c(-c3ccc(N(c4ccc(-c5ccc6c(ccc7oc8ccccc8c76)c5)cc4)c4ccc5c(c4)sc4ccccc45)cc3)cccc2c1] 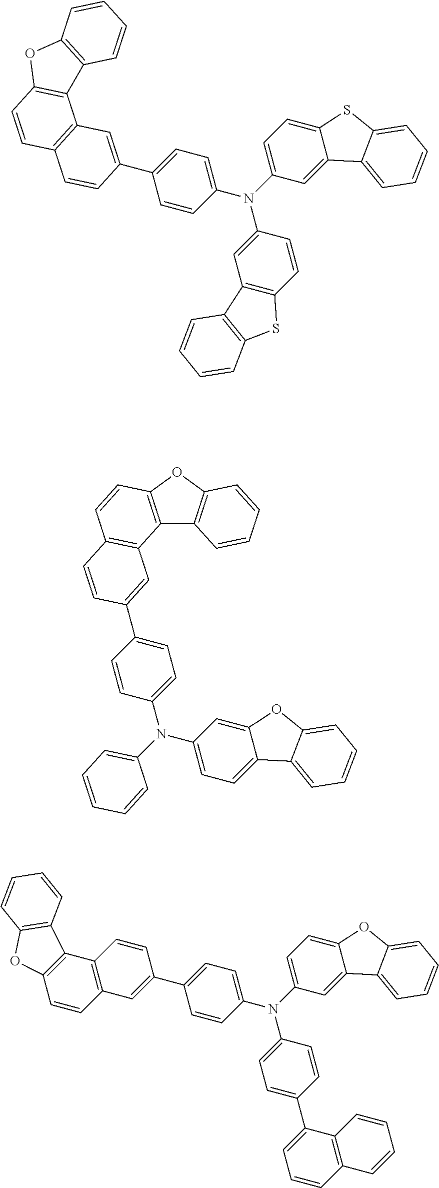 [SMILES: c1ccc2c(c1)oc1ccc3ccc(-c4ccc(N(c5ccc6sc7ccccc7c6c5)c5ccc6sc7ccccc7c6c5)cc4)cc3c12 ; c1ccc(N(c2ccc(-c3ccc4ccc5oc6ccccc6c5c4c3)cc2)c2ccc3c(c2)oc2ccccc23)cc1 ; c1ccc2c(-c3ccc(N(c4ccc(-c5ccc6c(ccc7oc8ccccc8c76)c5)cc4)c4ccc5oc6ccccc6c5c4)cc3)cccc2c1] 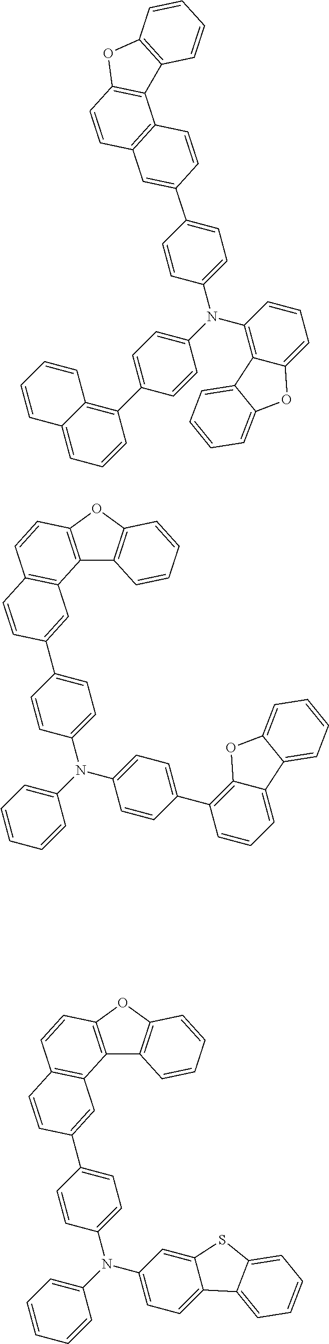 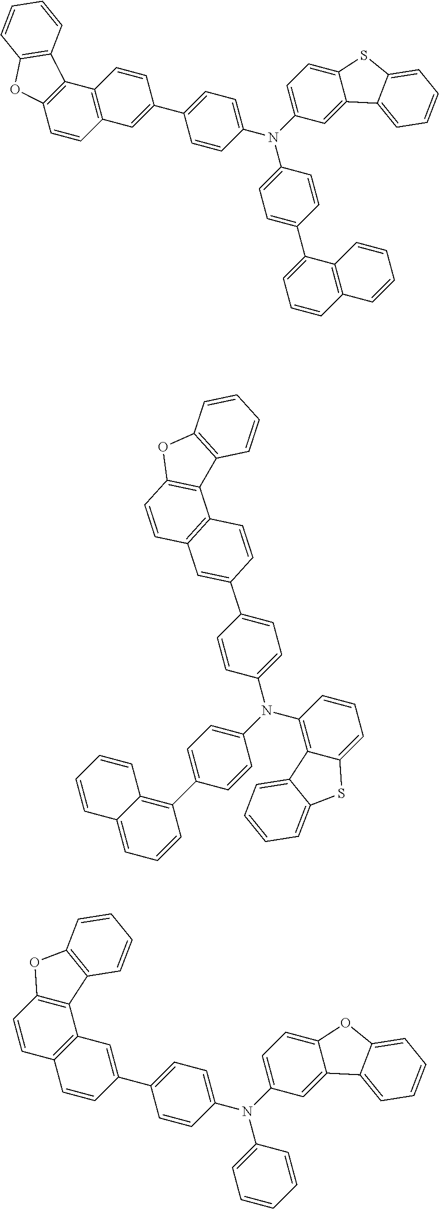 [SMILES: c1ccc2c(-c3ccc(N(c4ccc(-c5ccc6c(ccc7oc8ccccc8c76)c5)cc4)c4ccc5sc6ccccc6c5c4)cc3)cccc2c1 ; c1ccc2c(-c3ccc(N(c4ccc(-c5ccc6c(ccc7oc8ccccc8c76)c5)cc4)c4cccc5sc6ccccc6c45)cc3)cccc2c1 ; c1ccc(N(c2ccc(-c3ccc4ccc5oc6ccccc6c5c4c3)cc2)c2ccc3oc4ccccc4c3c2)cc1] 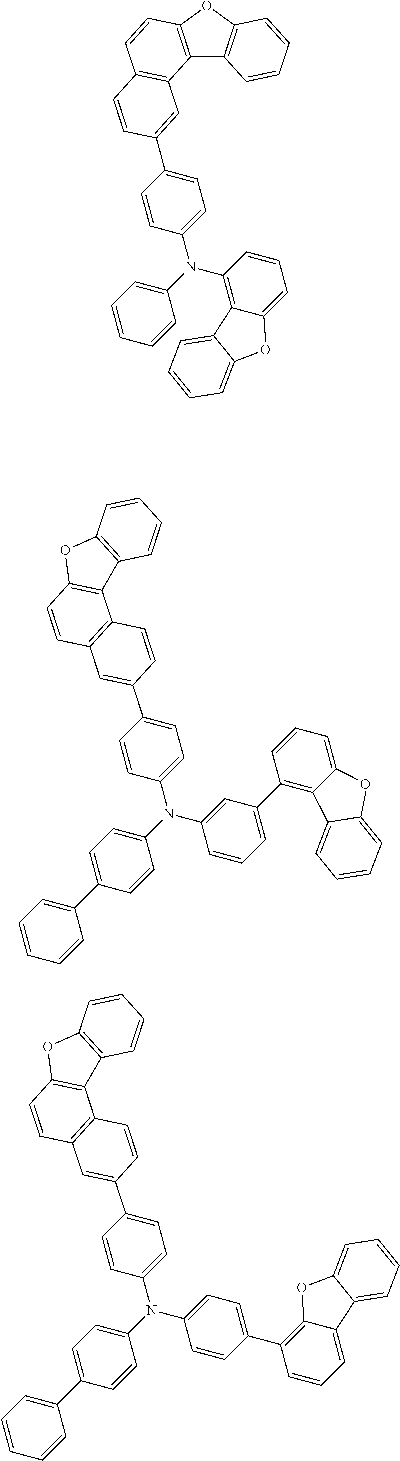 [SMILES: c1ccc(N(c2ccc(-c3ccc4ccc5oc6ccccc6c5c4c3)cc2)c2cccc3oc4ccccc4c23)cc1 ; c1ccc(-c2ccc(N(c3ccc(-c4ccc5c(ccc6oc7ccccc7c65)c4)cc3)c3cccc(-c4cccc5oc6ccccc6c45)c3)cc2)cc1 ; c1ccc(-c2ccc(N(c3ccc(-c4ccc5c(ccc6oc7ccccc7c65)c4)cc3)c3ccc(-c4cccc5c4oc4ccccc45)cc3)cc2)cc1] 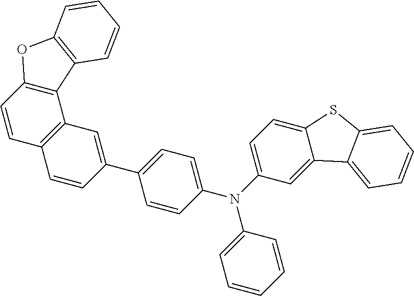 [SMILES: c1ccc(N(c2ccc(-c3ccc4ccc5oc6ccccc6c5c4c3)cc2)c2ccc3sc4ccccc4c3c2)cc1] 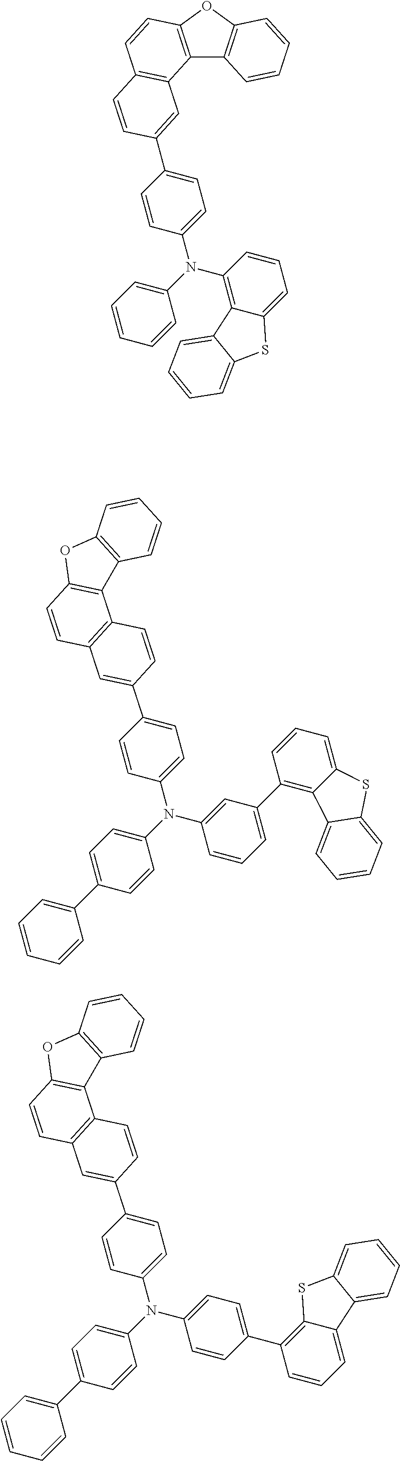 [SMILES: c1ccc(N(c2ccc(-c3ccc4ccc5oc6ccccc6c5c4c3)cc2)c2cccc3sc4ccccc4c23)cc1 ; c1ccc(-c2ccc(N(c3ccc(-c4ccc5c(ccc6oc7ccccc7c65)c4)cc3)c3cccc(-c4cccc5sc6ccccc6c45)c3)cc2)cc1 ; c1ccc(-c2ccc(N(c3ccc(-c4ccc5c(ccc6oc7ccccc7c65)c4)cc3)c3ccc(-c4cccc5c4sc4ccccc45)cc3)cc2)cc1] 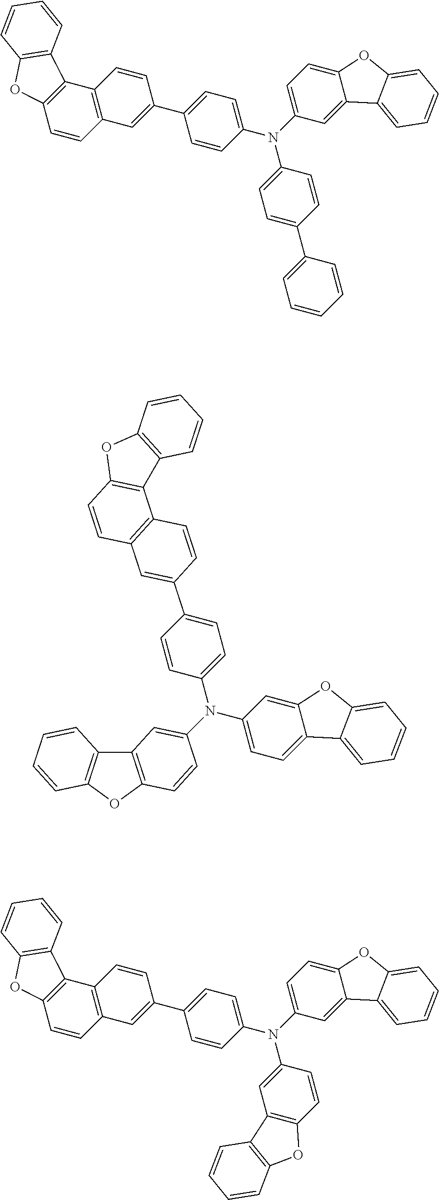 [SMILES: c1ccc(-c2ccc(N(c3ccc(-c4ccc5c(ccc6oc7ccccc7c65)c4)cc3)c3ccc4oc5ccccc5c4c3)cc2)cc1 ; c1ccc2c(c1)oc1cc(N(c3ccc(-c4ccc5c(ccc6oc7ccccc7c65)c4)cc3)c3ccc4oc5ccccc5c4c3)ccc12 ; c1ccc2c(c1)oc1ccc(N(c3ccc(-c4ccc5c(ccc6oc7ccccc7c65)c4)cc3)c3ccc4oc5ccccc5c4c3)cc12] 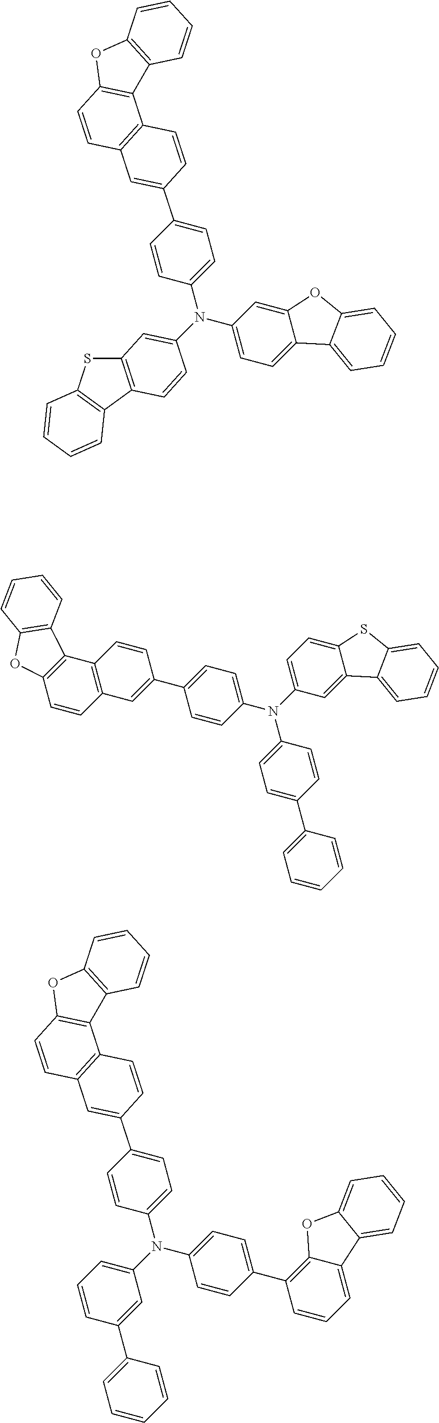 [SMILES: c1ccc2c(c1)oc1cc(N(c3ccc(-c4ccc5c(ccc6oc7ccccc7c65)c4)cc3)c3ccc4c(c3)sc3ccccc34)ccc12 ; c1ccc(-c2ccc(N(c3ccc(-c4ccc5c(ccc6oc7ccccc7c65)c4)cc3)c3ccc4sc5ccccc5c4c3)cc2)cc1 ; c1ccc(-c2cccc(N(c3ccc(-c4ccc5c(ccc6oc7ccccc7c65)c4)cc3)c3ccc(-c4cccc5c4oc4ccccc45)cc3)c2)cc1] 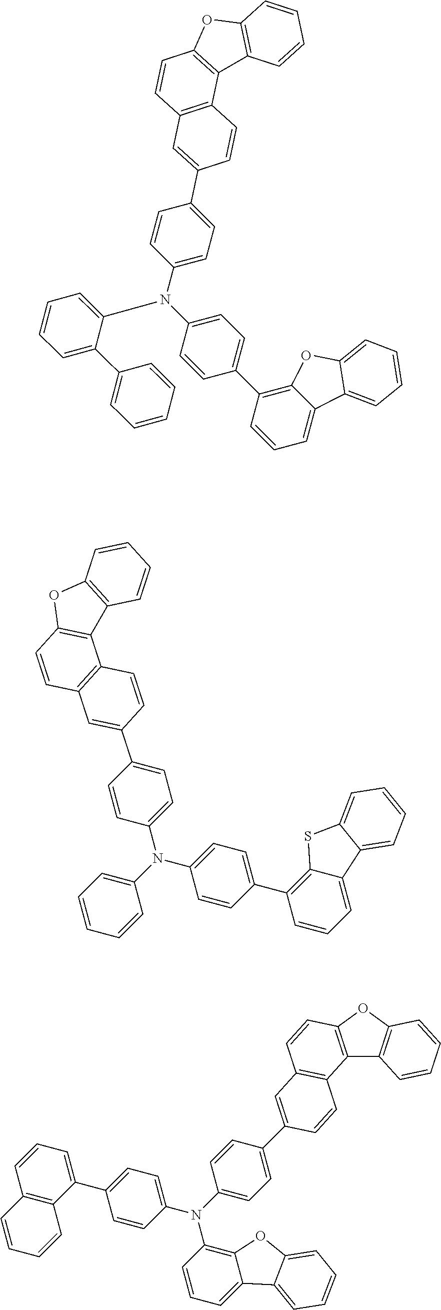 [SMILES: c1ccc(-c2ccccc2N(c2ccc(-c3ccc4c(ccc5oc6ccccc6c54)c3)cc2)c2ccc(-c3cccc4c3oc3ccccc34)cc2)cc1 ; c1ccc(N(c2ccc(-c3ccc4c(ccc5oc6ccccc6c54)c3)cc2)c2ccc(-c3cccc4c3sc3ccccc34)cc2)cc1 ; c1ccc2c(-c3ccc(N(c4ccc(-c5ccc6c(ccc7oc8ccccc8c76)c5)cc4)c4cccc5c4oc4ccccc45)cc3)cccc2c1] 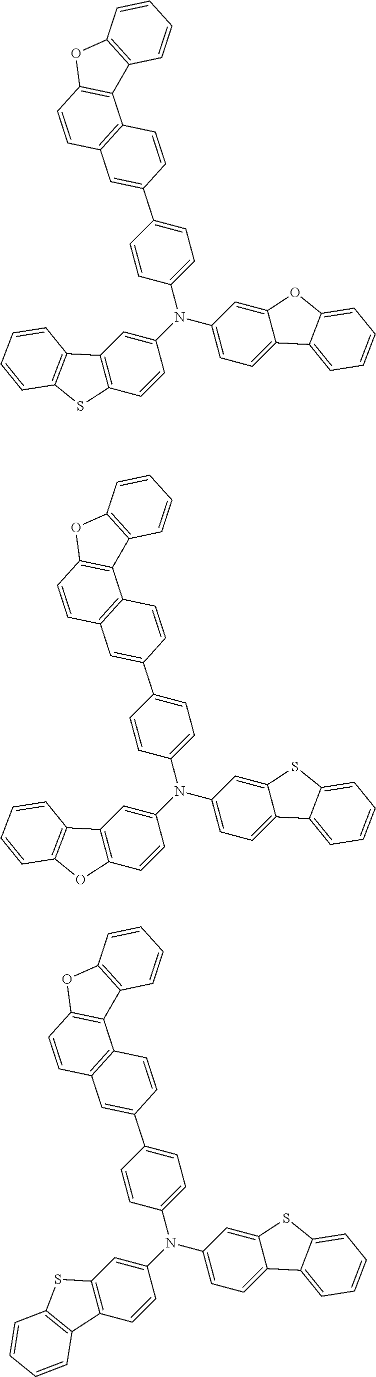 [SMILES: c1ccc2c(c1)oc1cc(N(c3ccc(-c4ccc5c(ccc6oc7ccccc7c65)c4)cc3)c3ccc4sc5ccccc5c4c3)ccc12 ; c1ccc2c(c1)oc1ccc(N(c3ccc(-c4ccc5c(ccc6oc7ccccc7c65)c4)cc3)c3ccc4c(c3)sc3ccccc34)cc12 ; c1ccc2c(c1)oc1ccc3cc(-c4ccc(N(c5ccc6c(c5)sc5ccccc56)c5ccc6c(c5)sc5ccccc56)cc4)ccc3c12] 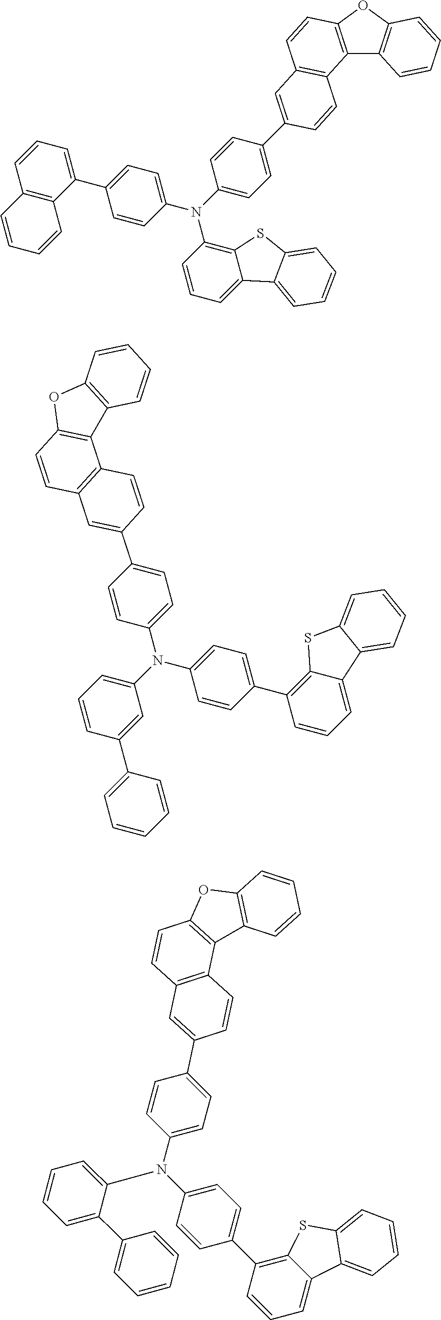 [SMILES: c1ccc2c(-c3ccc(N(c4ccc(-c5ccc6c(ccc7oc8ccccc8c76)c5)cc4)c4cccc5c4sc4ccccc45)cc3)cccc2c1 ; c1ccc(-c2cccc(N(c3ccc(-c4ccc5c(ccc6oc7ccccc7c65)c4)cc3)c3ccc(-c4cccc5c4sc4ccccc45)cc3)c2)cc1 ; c1ccc(-c2ccccc2N(c2ccc(-c3ccc4c(ccc5oc6ccccc6c54)c3)cc2)c2ccc(-c3cccc4c3sc3ccccc34)cc2)cc1] 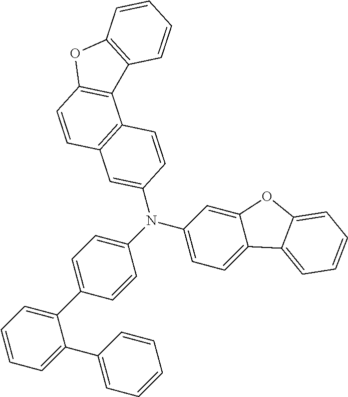 [SMILES: c1ccc(-c2ccccc2-c2ccc(N(c3ccc4c(ccc5oc6ccccc6c54)c3)c3ccc4c(c3)oc3ccccc34)cc2)cc1] 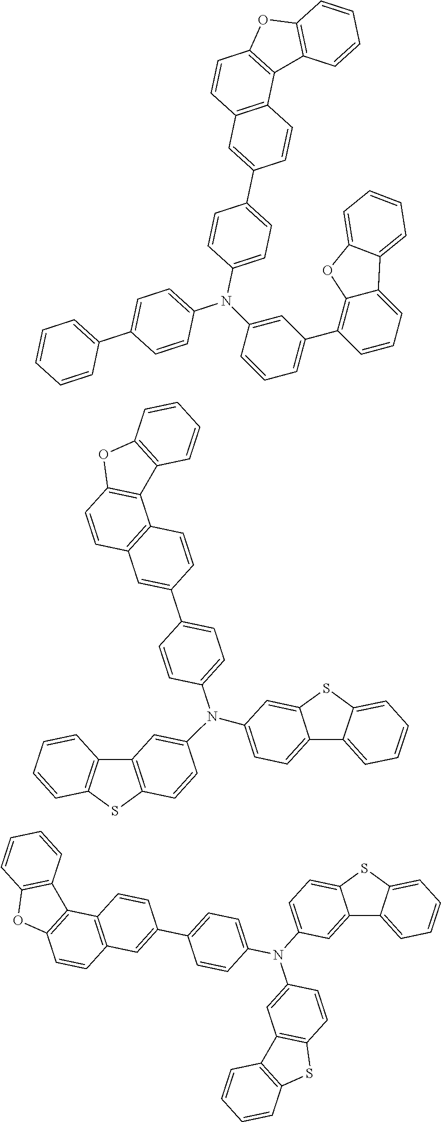 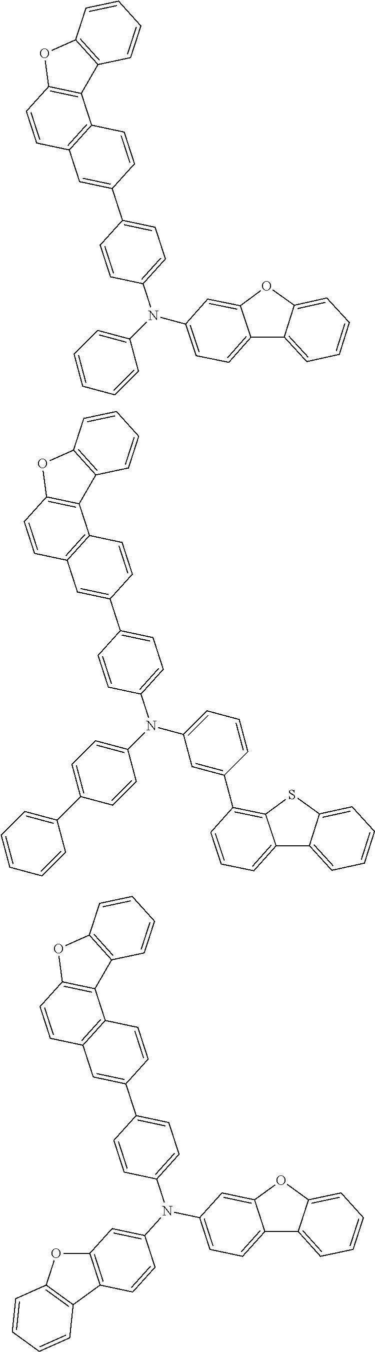 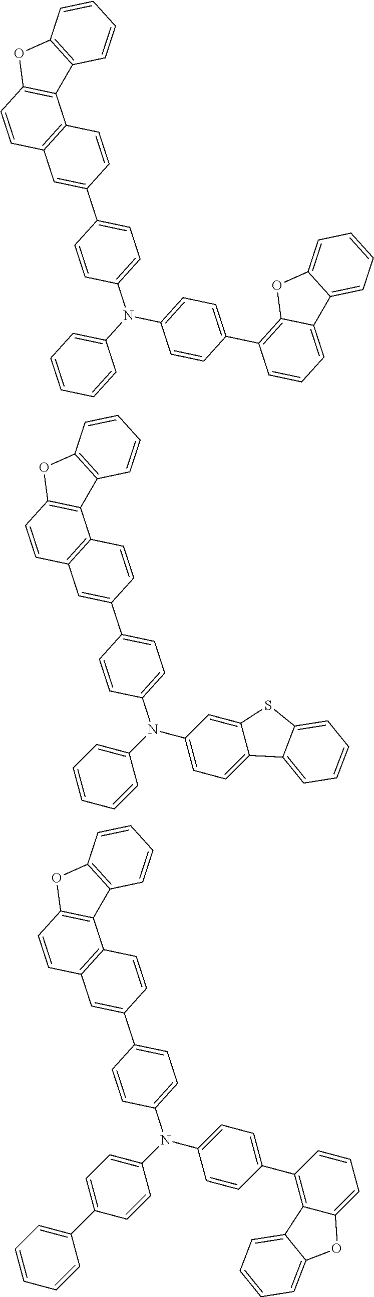 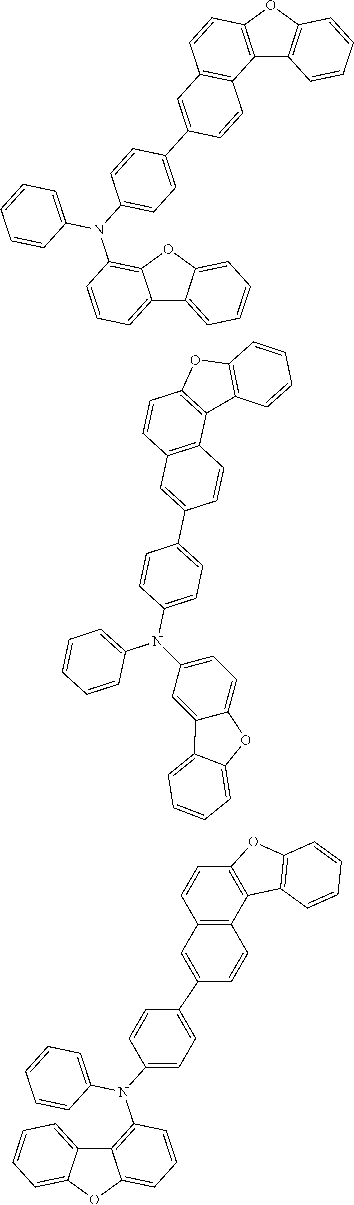 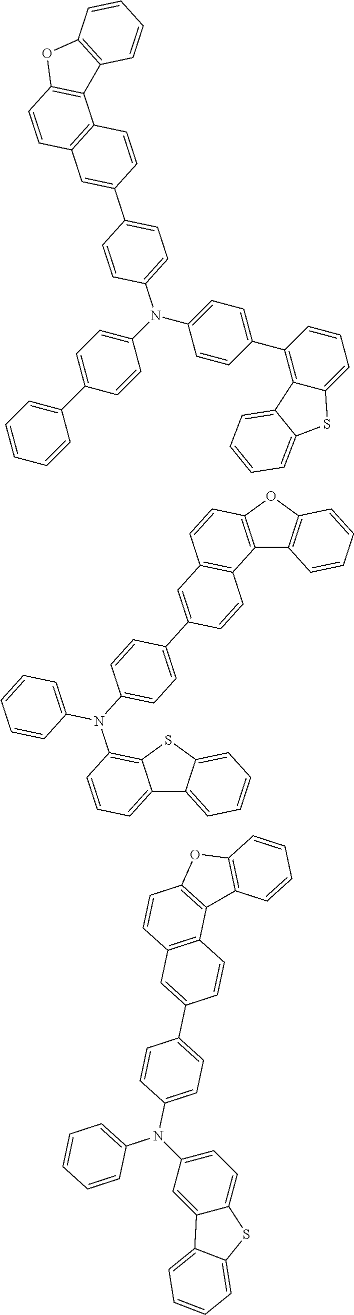 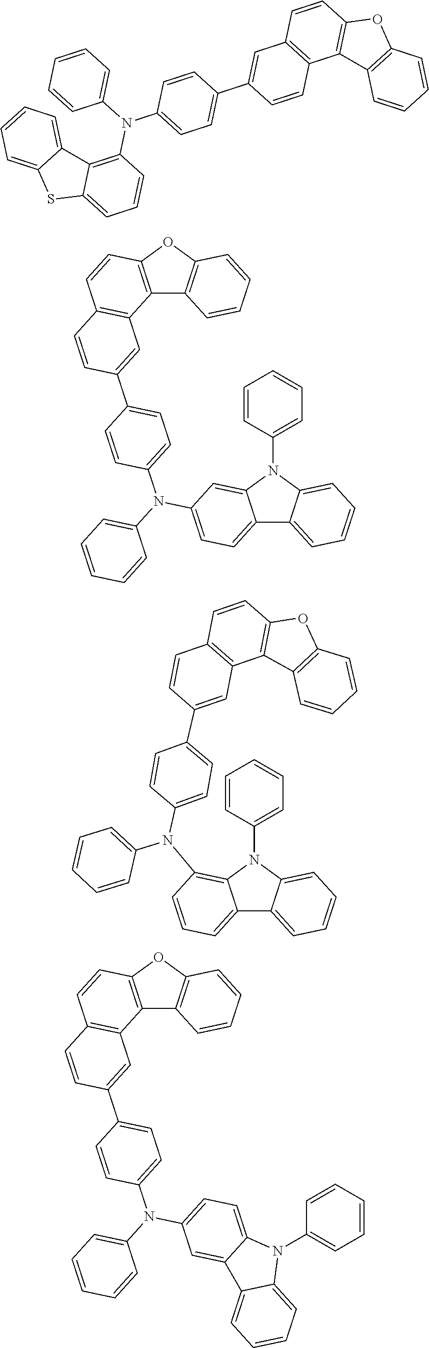 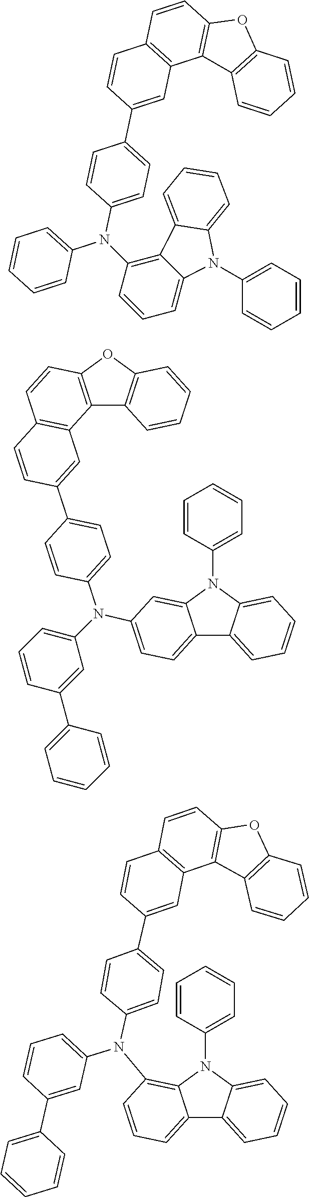 [SMILES: c1ccc(N(c2ccc(-c3ccc4ccc5oc6ccccc6c5c4c3)cc2)c2cccc3c2c2ccccc2n3-c2ccccc2)cc1 ; c1ccc(-c2cccc(N(c3ccc(-c4ccc5ccc6oc7ccccc7c6c5c4)cc3)c3ccc4c5ccccc5n(-c5ccccc5)c4c3)c2)cc1 ; c1ccc(-c2cccc(N(c3ccc(-c4ccc5ccc6oc7ccccc7c6c5c4)cc3)c3cccc4c5ccccc5n(-c5ccccc5)c34)c2)cc1] 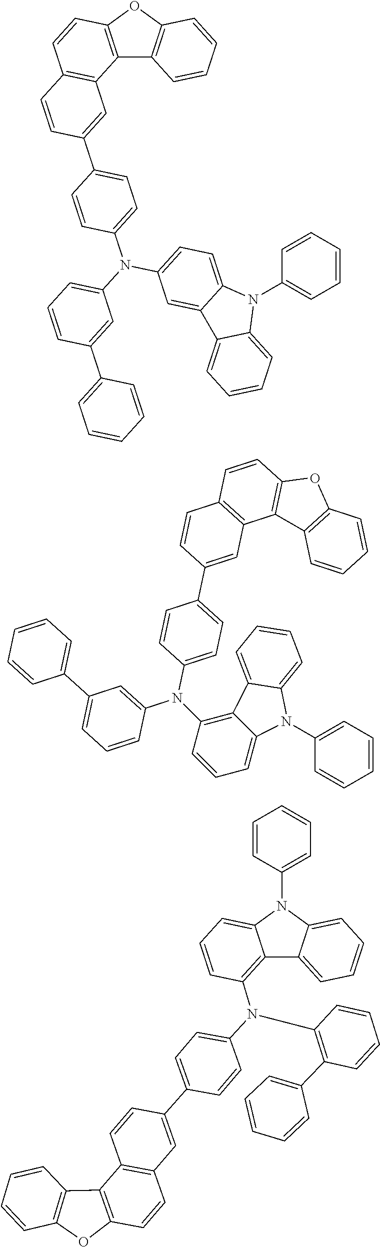 [SMILES: c1ccc(-c2cccc(N(c3ccc(-c4ccc5ccc6oc7ccccc7c6c5c4)cc3)c3ccc4c(c3)c3ccccc3n4-c3ccccc3)c2)cc1 ; c1ccc(-c2cccc(N(c3ccc(-c4ccc5ccc6oc7ccccc7c6c5c4)cc3)c3cccc4c3c3ccccc3n4-c3ccccc3)c2)cc1 ; c1ccc(-c2ccccc2N(c2ccc(-c3ccc4c(ccc5oc6ccccc6c54)c3)cc2)c2cccc3c2c2ccccc2n3-c2ccccc2)cc1] 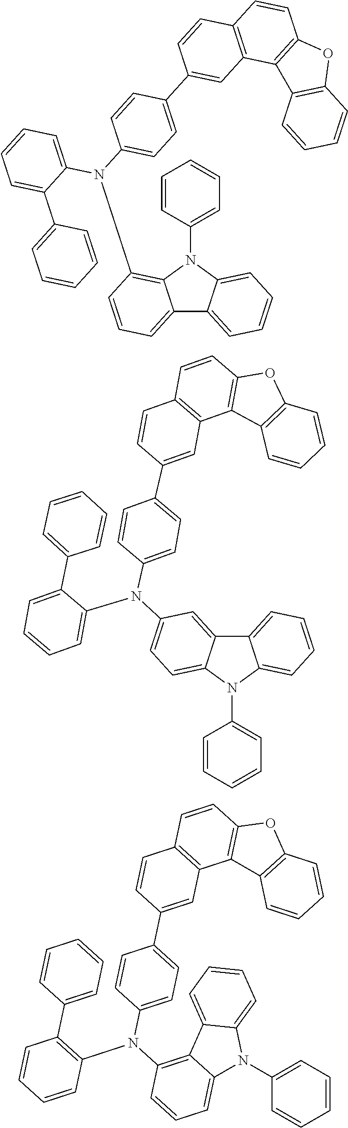 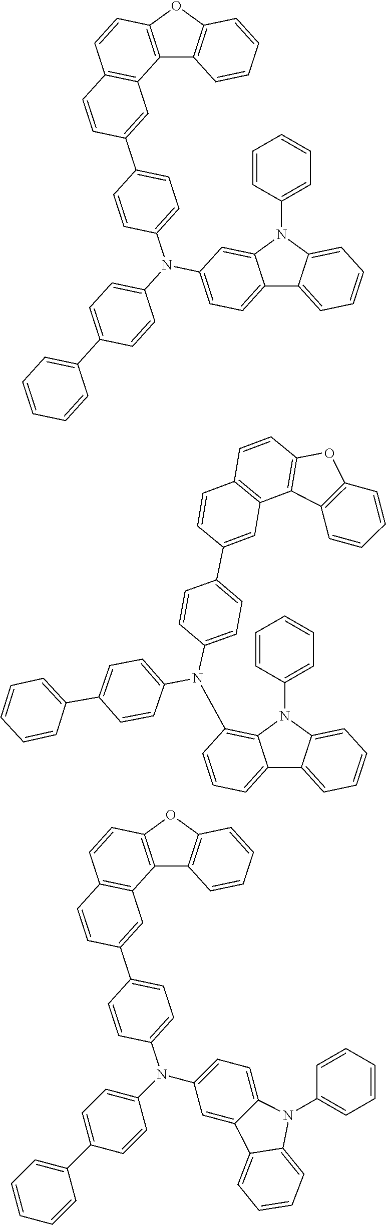 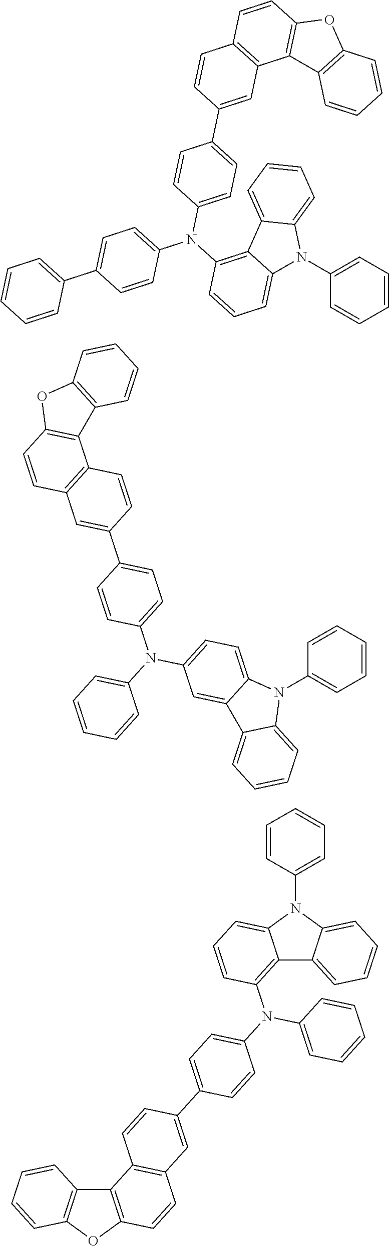 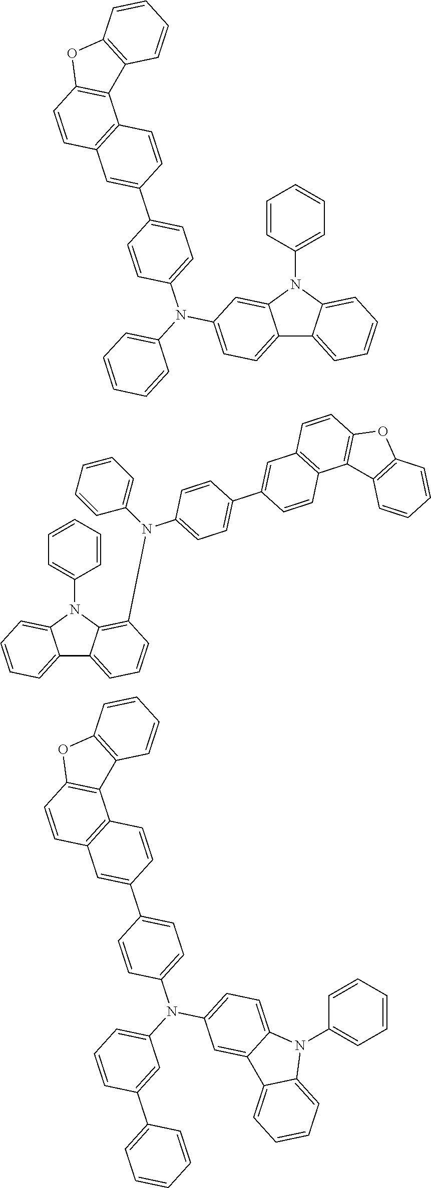 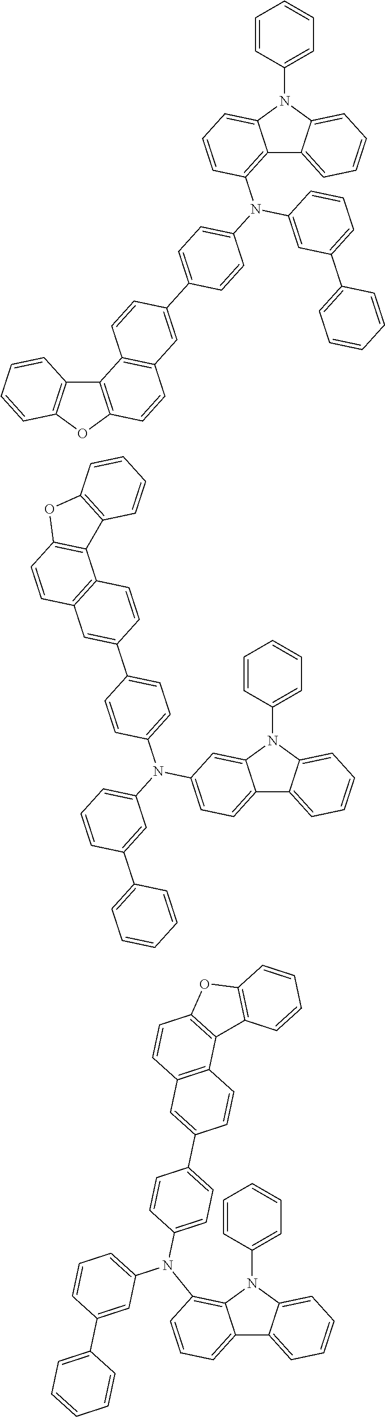 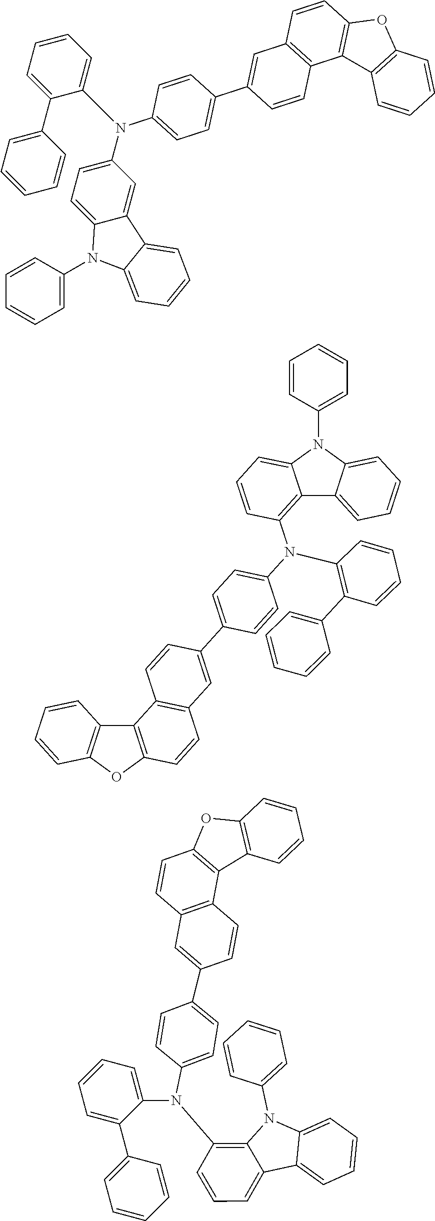 [SMILES: c1ccc(-c2ccccc2N(c2ccc(-c3ccc4c(ccc5oc6ccccc6c54)c3)cc2)c2ccc3c(c2)c2ccccc2n3-c2ccccc2)cc1 ; c1ccc(-c2ccccc2N(c2ccc(-c3ccc4c(ccc5oc6ccccc6c54)c3)cc2)c2cccc3c2c2ccccc2n3-c2ccccc2)cc1 ; c1ccc(-c2ccccc2N(c2ccc(-c3ccc4c(ccc5oc6ccccc6c54)c3)cc2)c2cccc3c4ccccc4n(-c4ccccc4)c23)cc1] 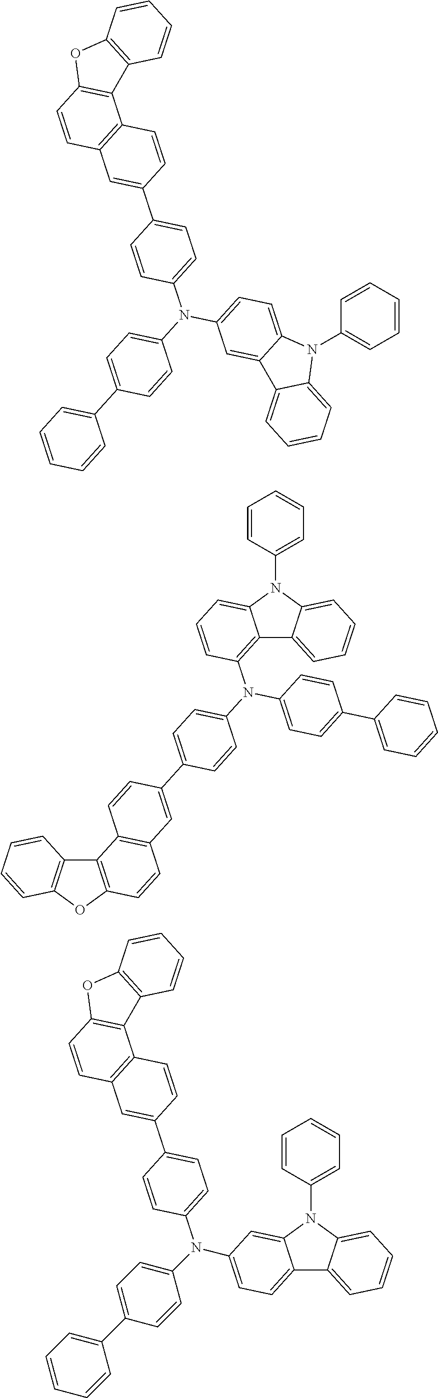 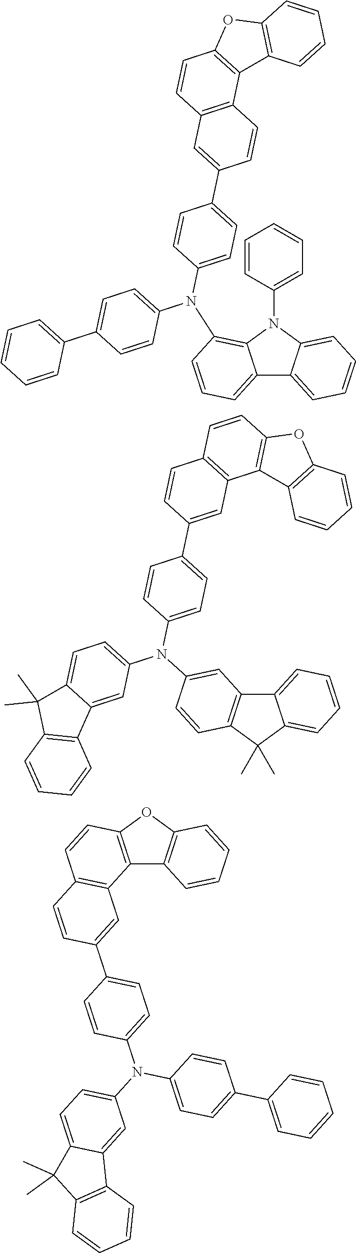 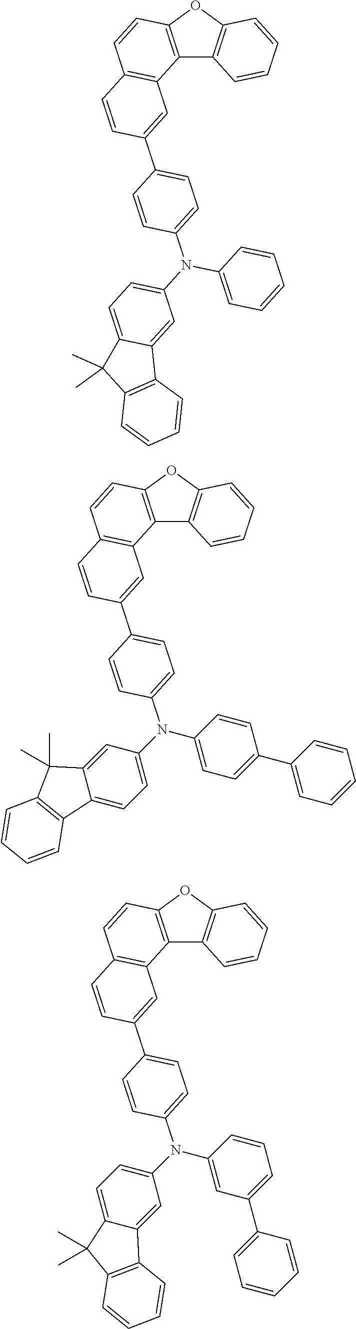 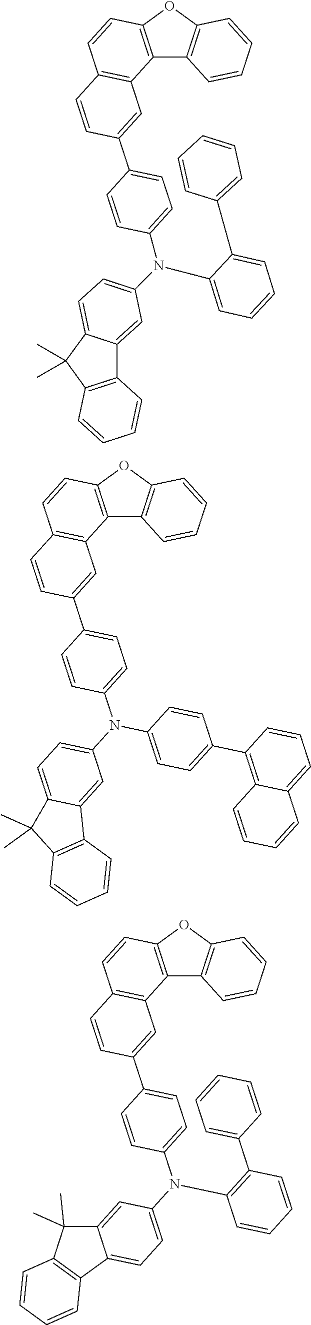 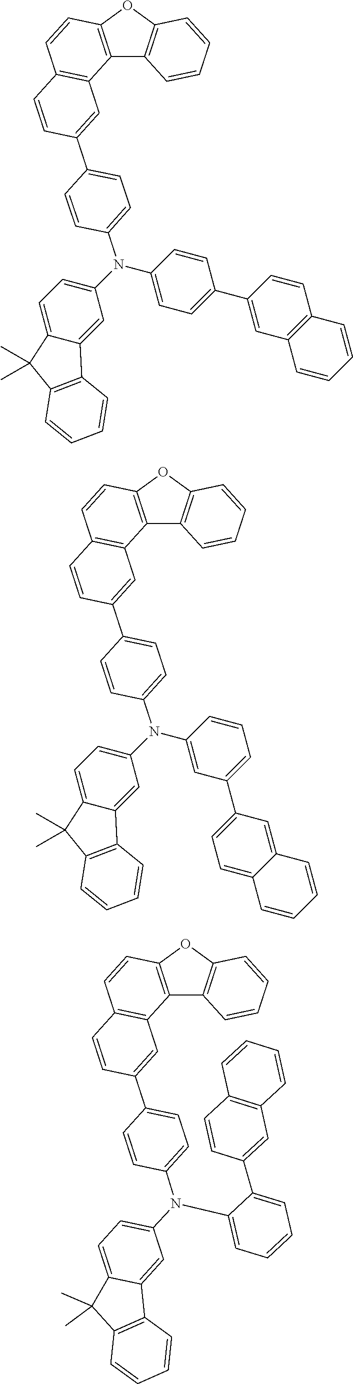 [SMILES: CC1(C)c2ccccc2-c2cc(N(c3ccc(-c4ccc5ccccc5c4)cc3)c3ccc(-c4ccc5ccc6oc7ccccc7c6c5c4)cc3)ccc21 ; CC1(C)c2ccccc2-c2cc(N(c3ccc(-c4ccc5ccc6oc7ccccc7c6c5c4)cc3)c3cccc(-c4ccc5ccccc5c4)c3)ccc21 ; CC1(C)c2ccccc2-c2cc(N(c3ccc(-c4ccc5ccc6oc7ccccc7c6c5c4)cc3)c3ccccc3-c3ccc4ccccc4c3)ccc21] 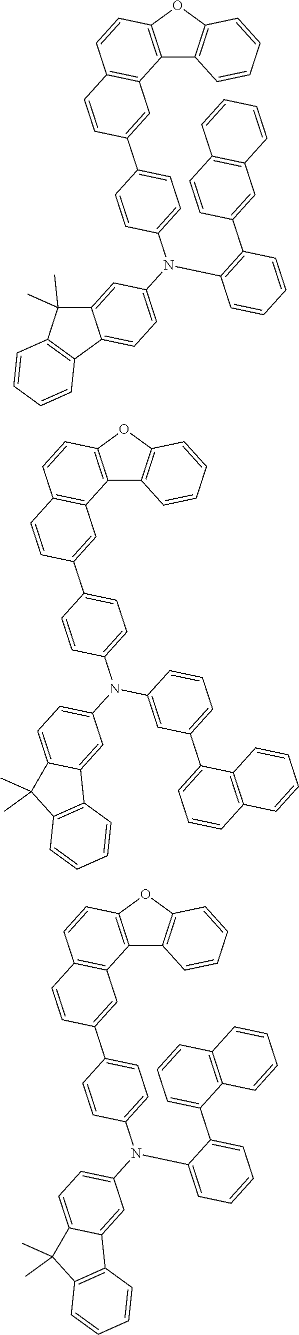 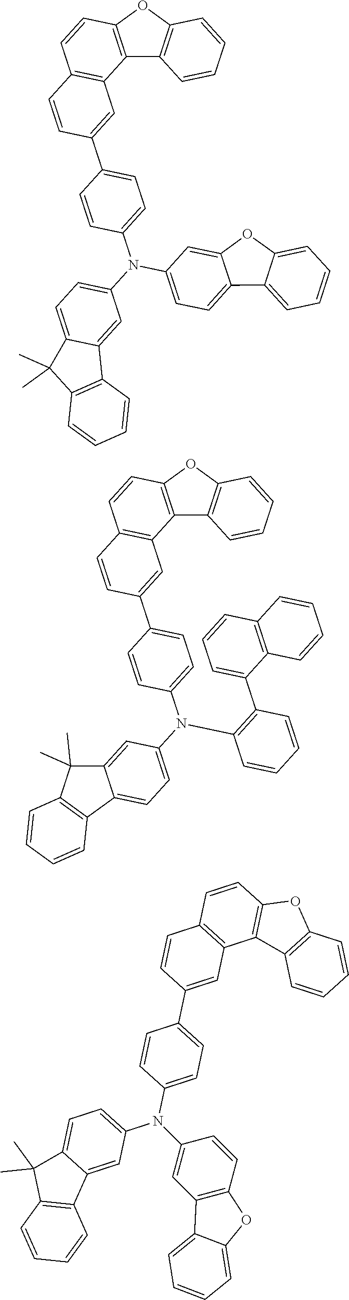 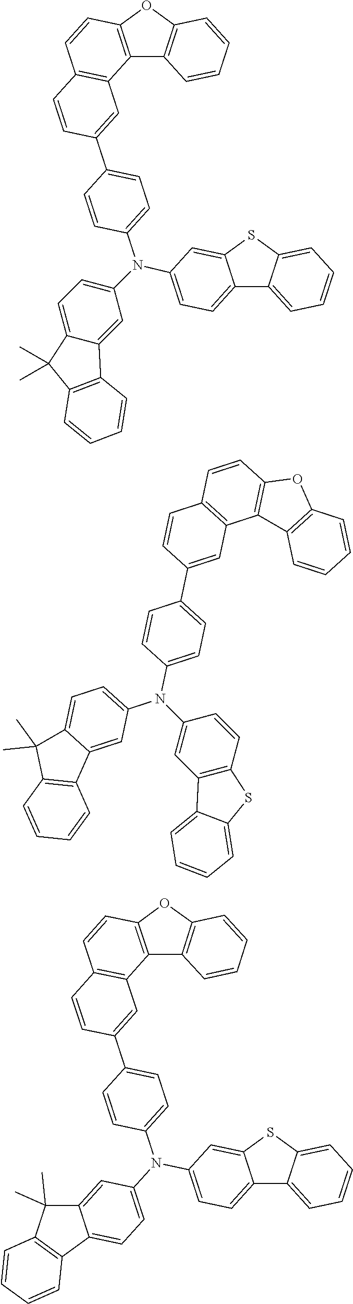 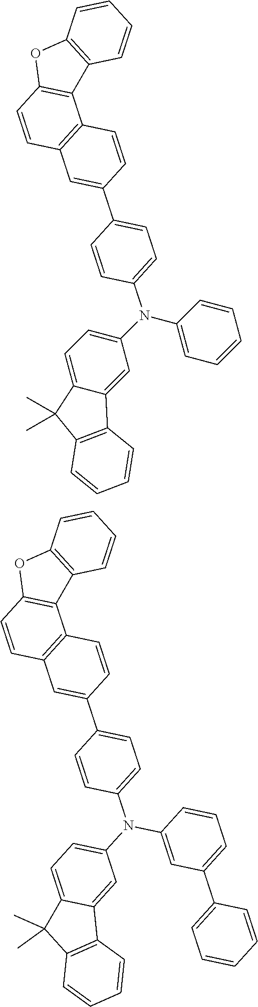 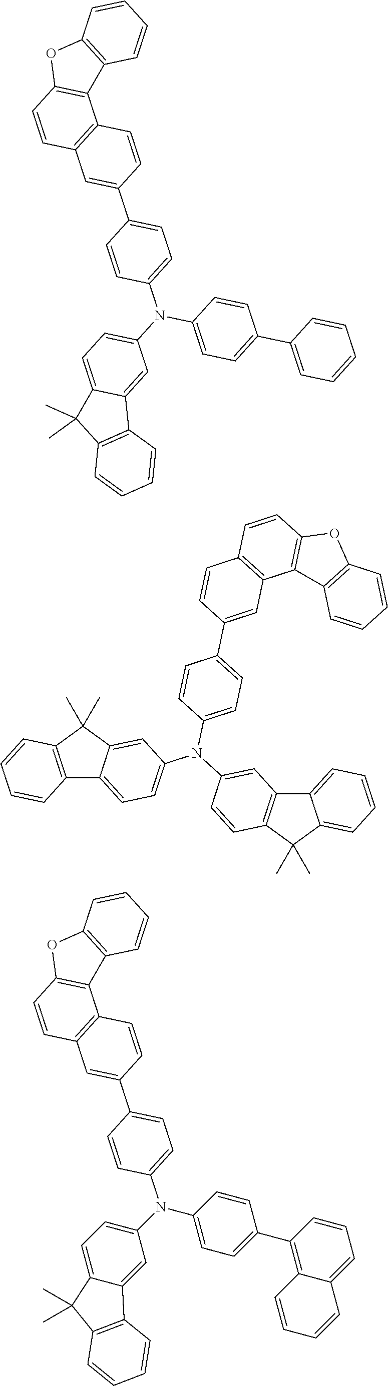 [SMILES: CC1(C)c2ccccc2-c2cc(N(c3ccc(-c4ccccc4)cc3)c3ccc(-c4ccc5c(ccc6oc7ccccc7c65)c4)cc3)ccc21 ; CC1(C)c2ccccc2-c2cc(N(c3ccc(-c4ccc5ccc6oc7ccccc7c6c5c4)cc3)c3ccc4c(c3)C(C)(C)c3ccccc3-4)ccc21 ; CC1(C)c2ccccc2-c2cc(N(c3ccc(-c4ccc5c(ccc6oc7ccccc7c65)c4)cc3)c3ccc(-c4cccc5ccccc45)cc3)ccc21] 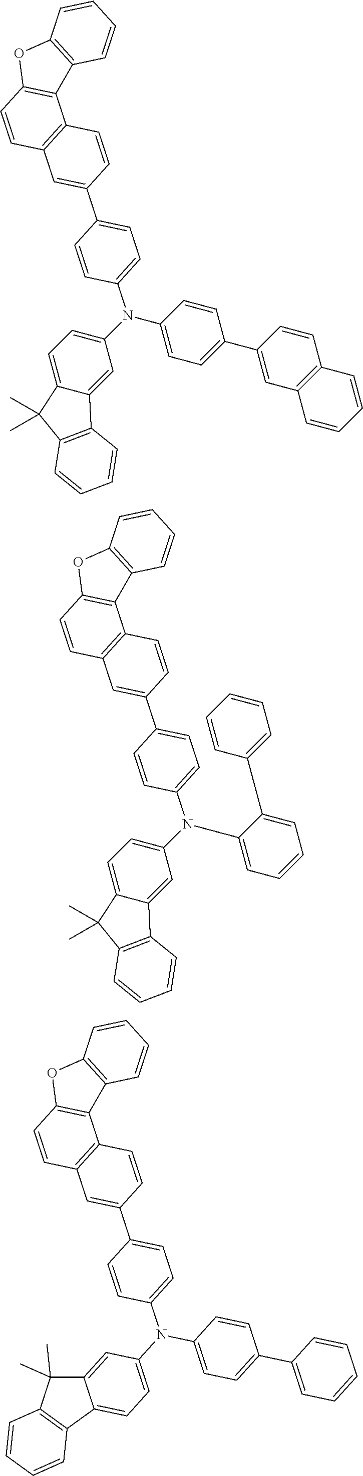 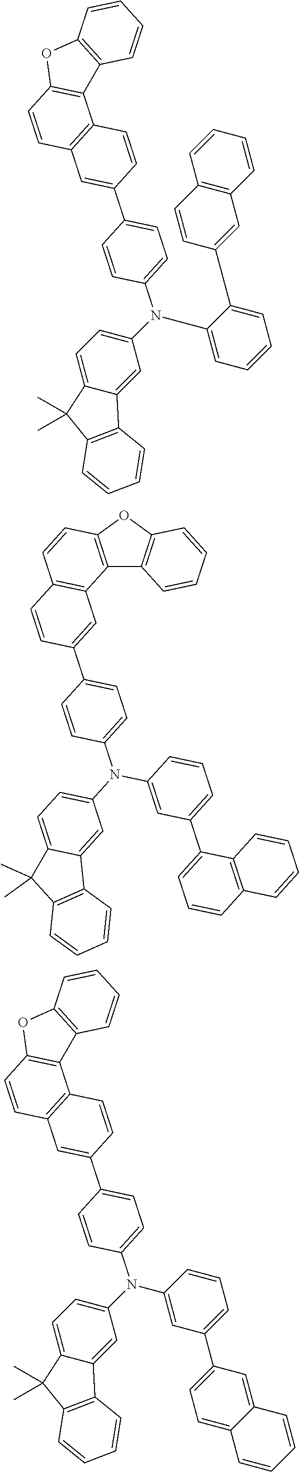 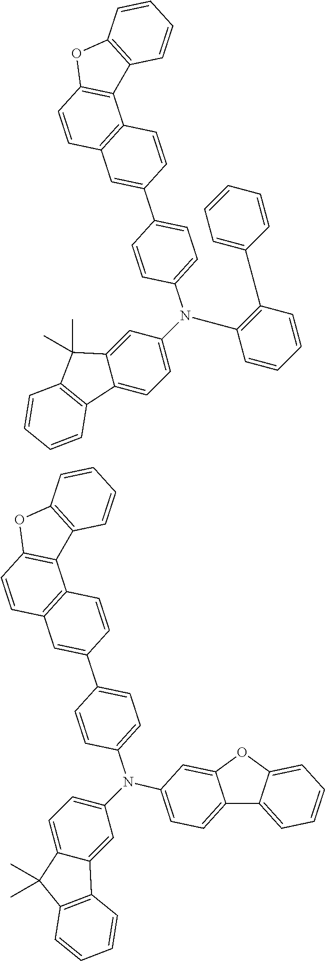 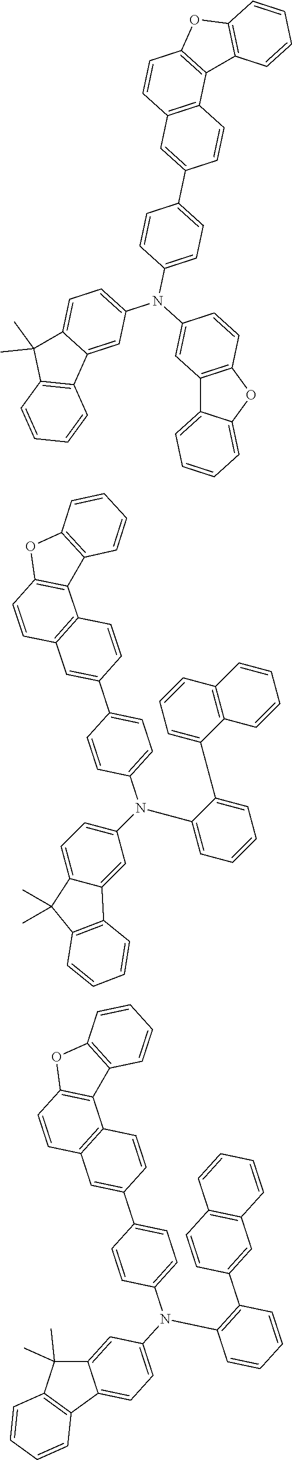 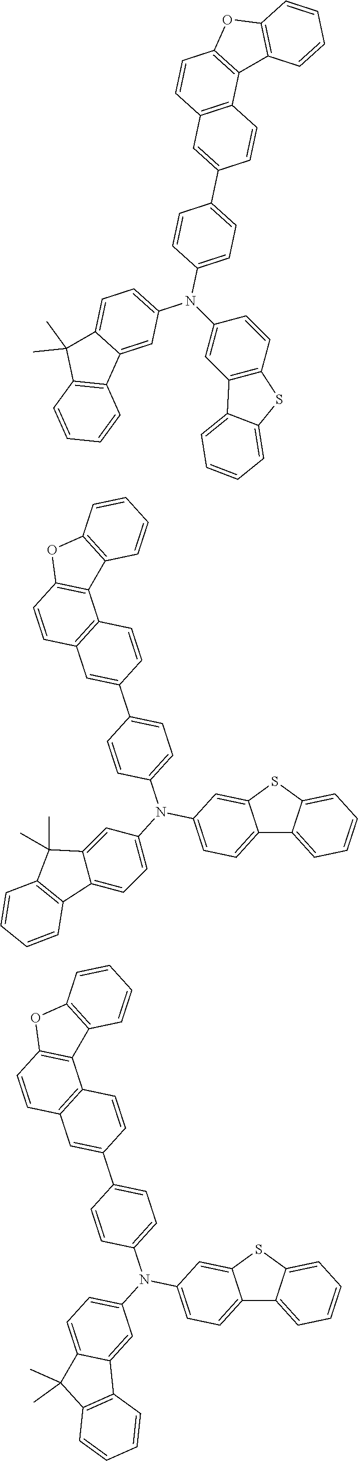 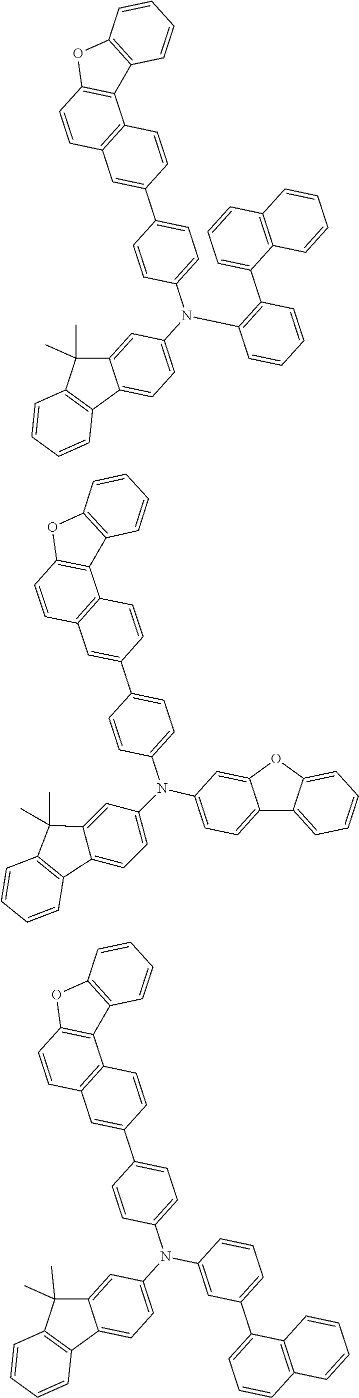 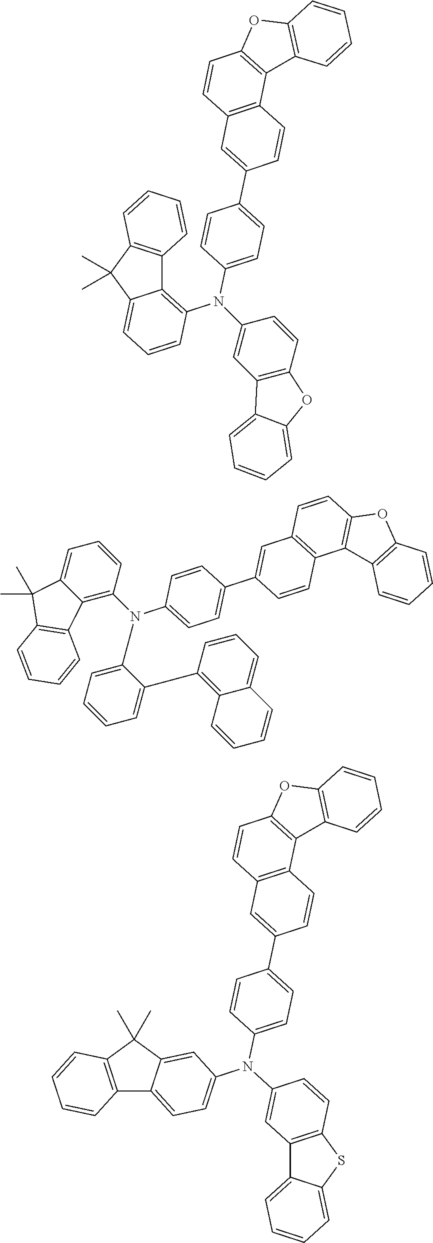 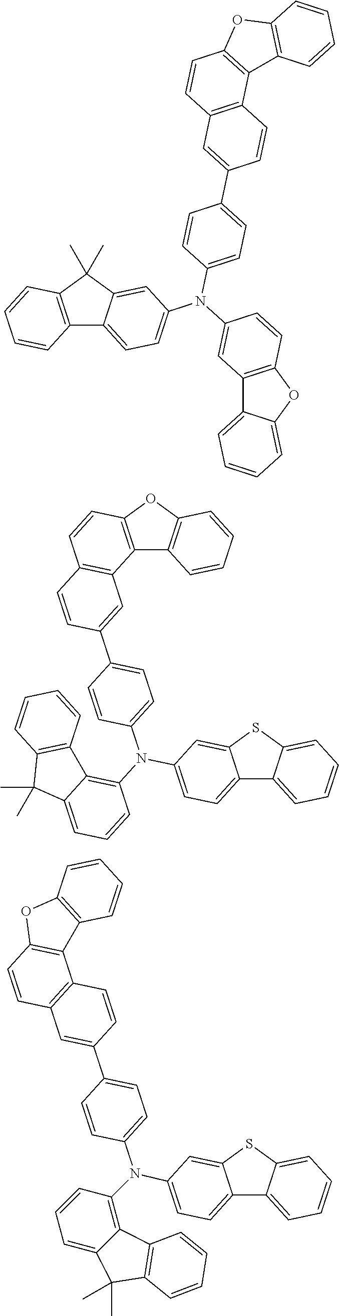 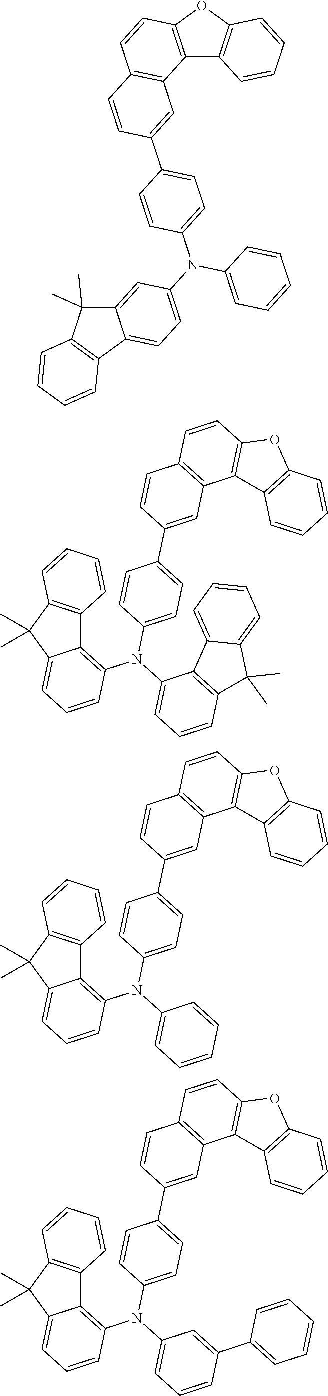 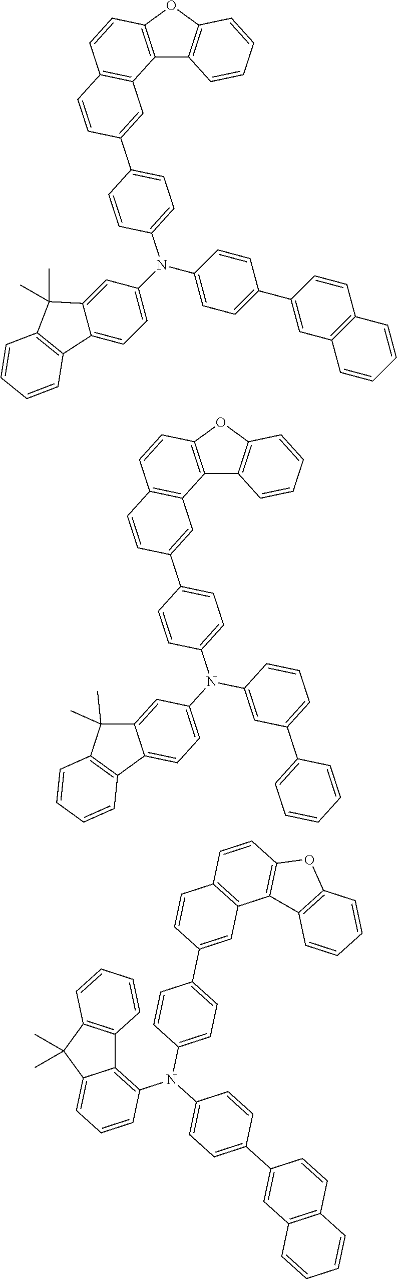 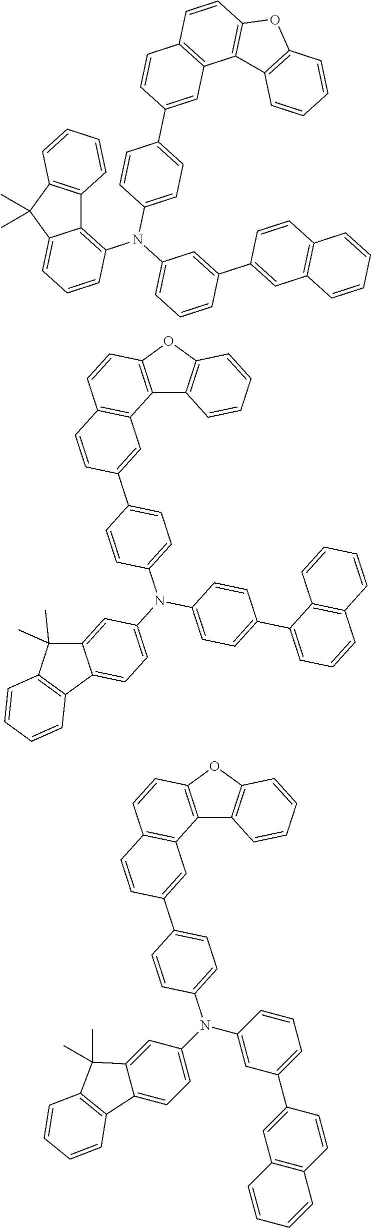 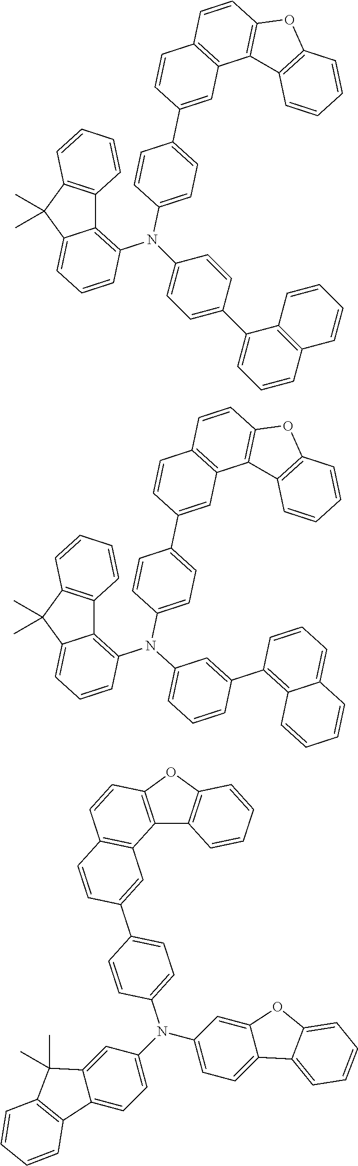 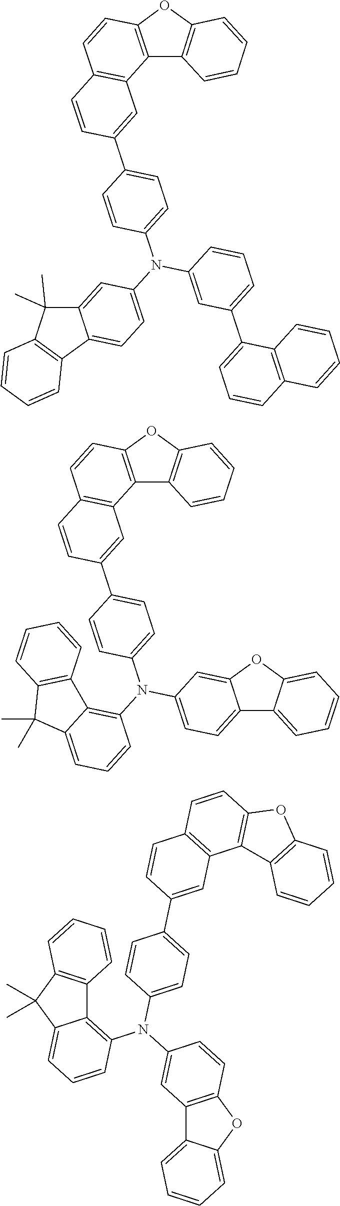 [SMILES: CC1(C)c2ccccc2-c2ccc(N(c3ccc(-c4ccc5ccc6oc7ccccc7c6c5c4)cc3)c3cccc(-c4cccc5ccccc45)c3)cc21 ; CC1(C)c2ccccc2-c2c(N(c3ccc(-c4ccc5ccc6oc7ccccc7c6c5c4)cc3)c3ccc4c(c3)oc3ccccc34)cccc21 ; CC1(C)c2ccccc2-c2c(N(c3ccc(-c4ccc5ccc6oc7ccccc7c6c5c4)cc3)c3ccc4oc5ccccc5c4c3)cccc21] 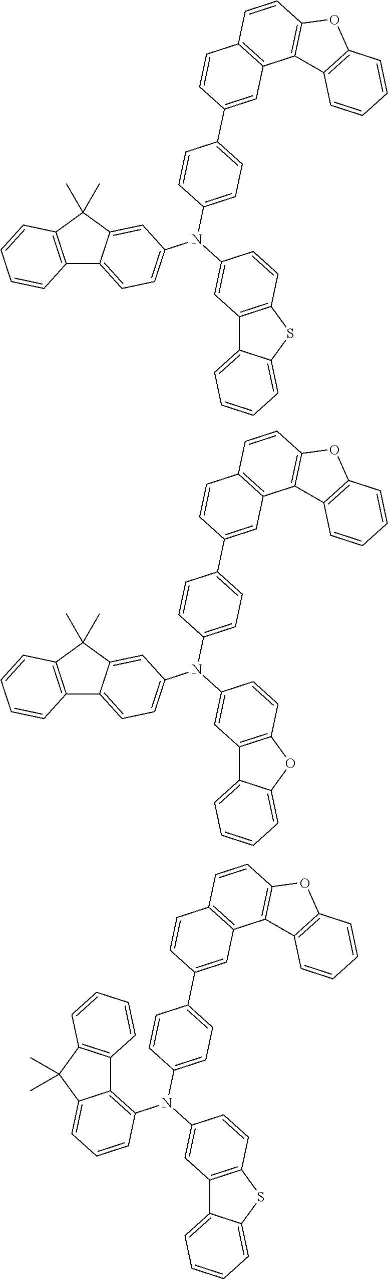 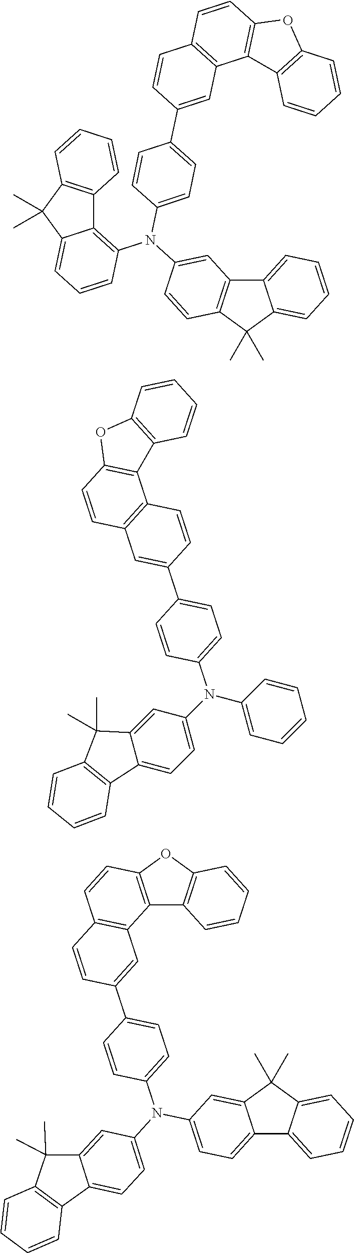 [SMILES: CC1(C)c2ccccc2-c2cc(N(c3ccc(-c4ccc5ccc6oc7ccccc7c6c5c4)cc3)c3cccc4c3-c3ccccc3C4(C)C)ccc21 ; CC1(C)c2ccccc2-c2ccc(N(c3ccccc3)c3ccc(-c4ccc5c(ccc6oc7ccccc7c65)c4)cc3)cc21 ; CC1(C)c2ccccc2-c2ccc(N(c3ccc(-c4ccc5ccc6oc7ccccc7c6c5c4)cc3)c3ccc4c(c3)C(C)(C)c3ccccc3-4)cc21] 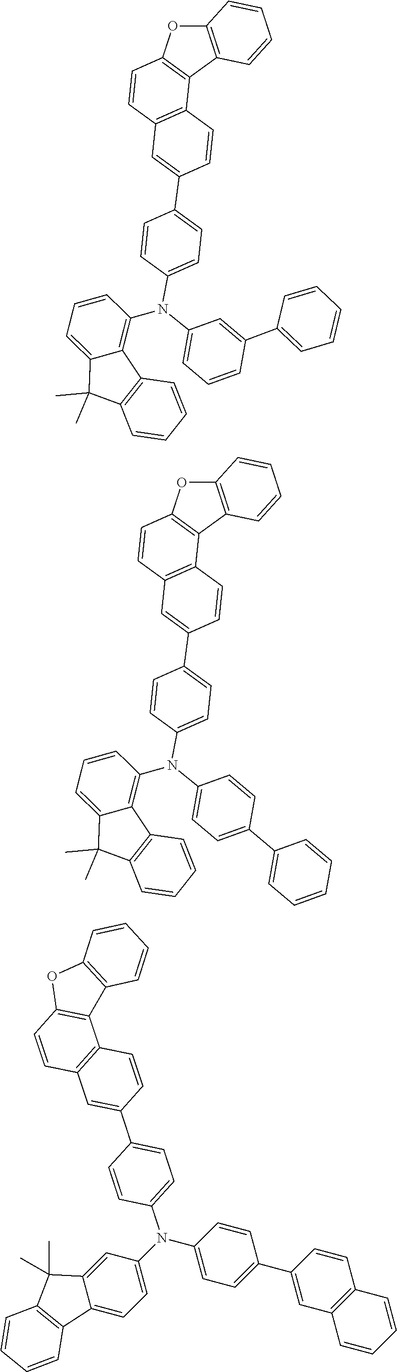 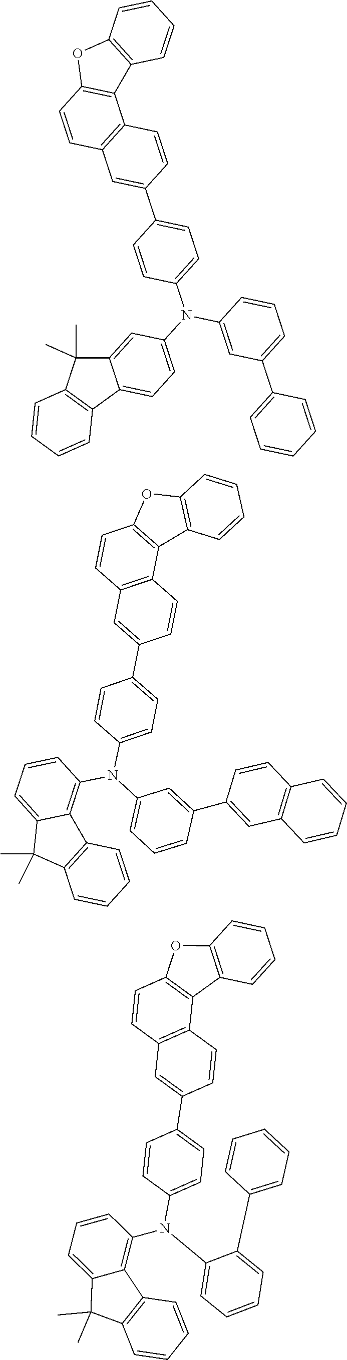 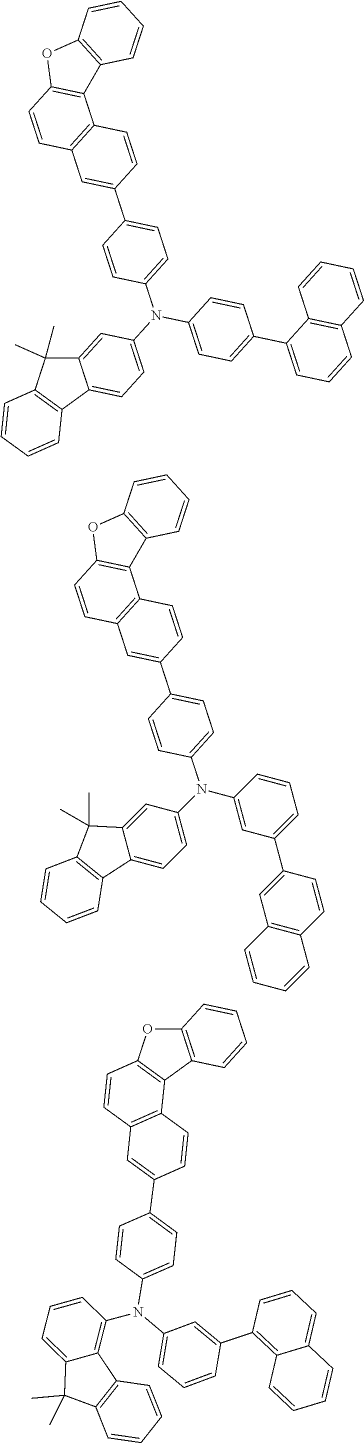 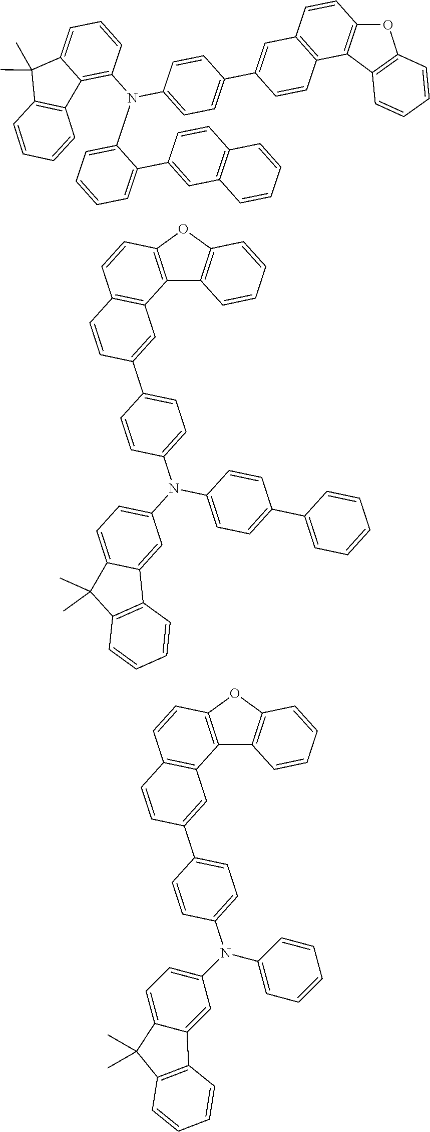 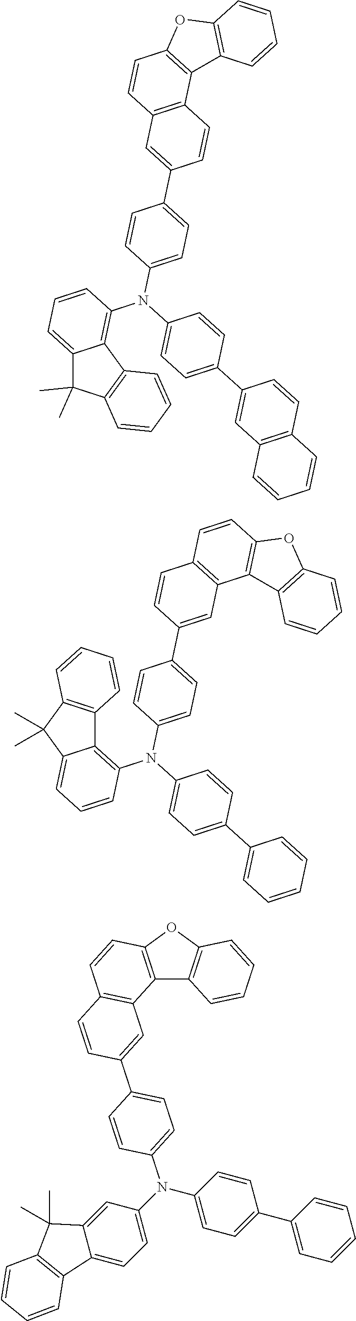 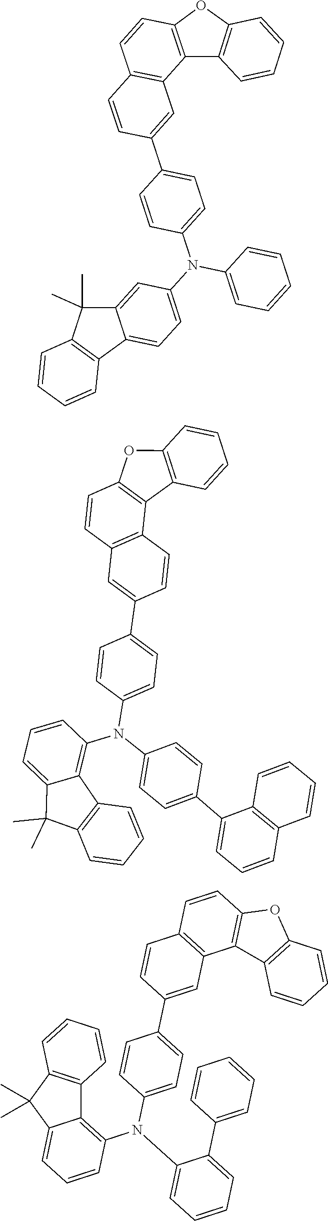 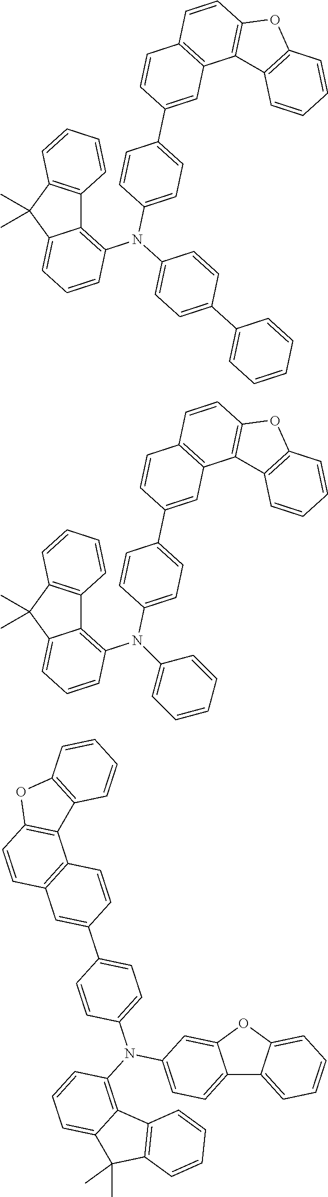 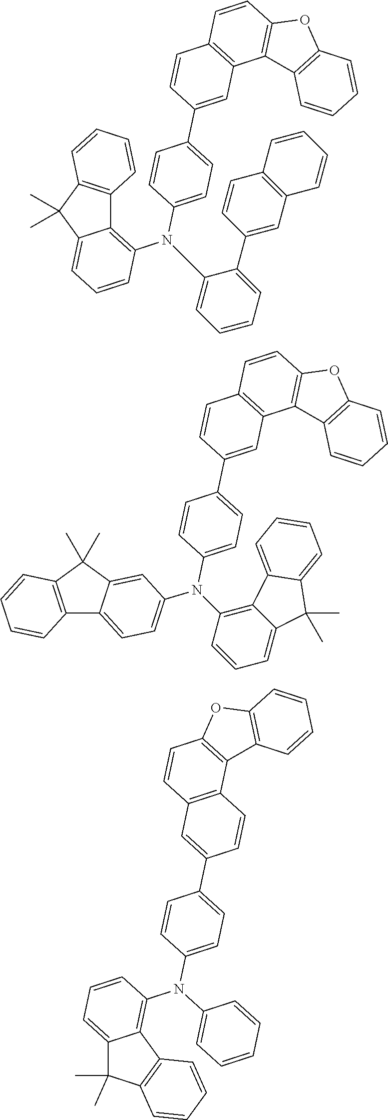 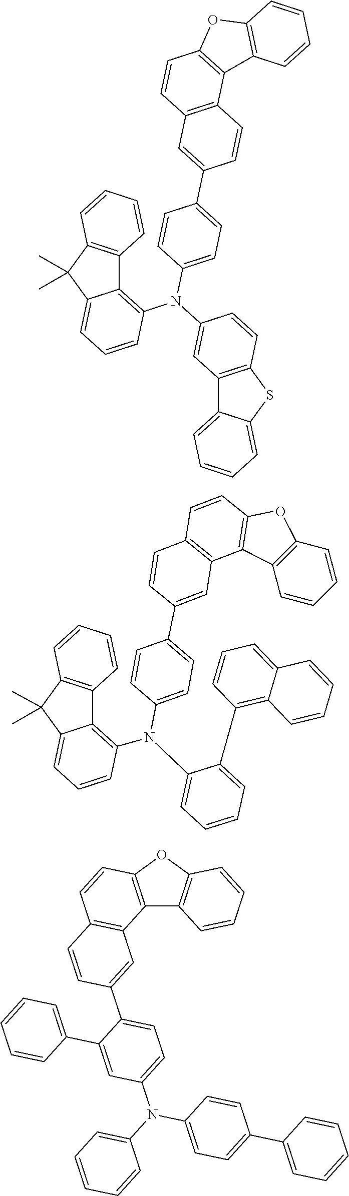 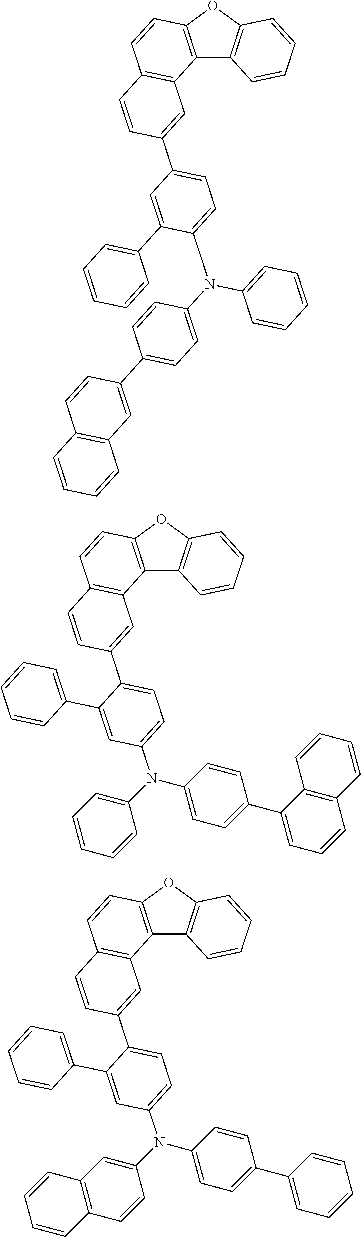 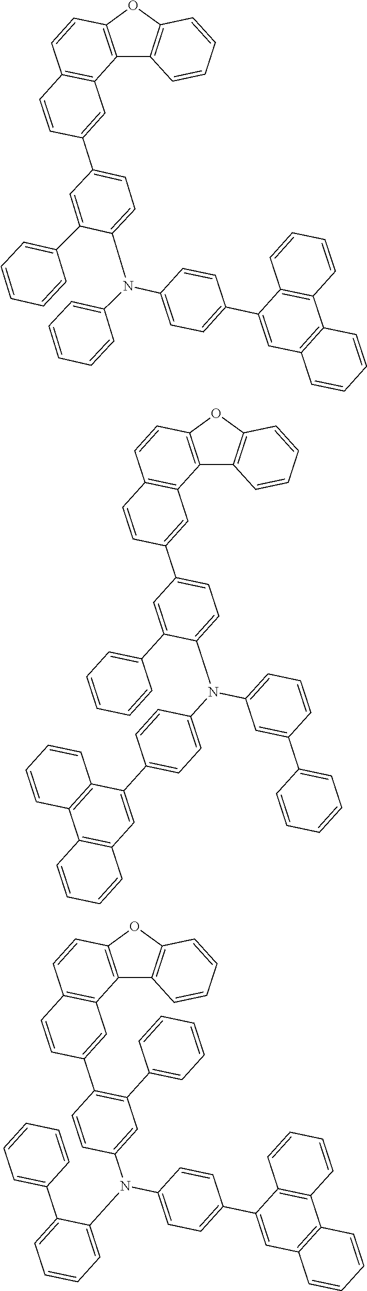 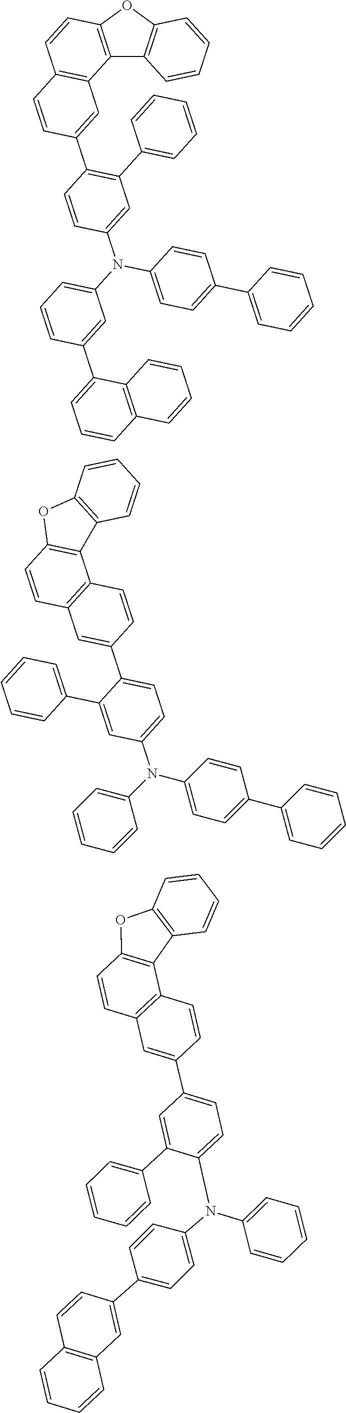 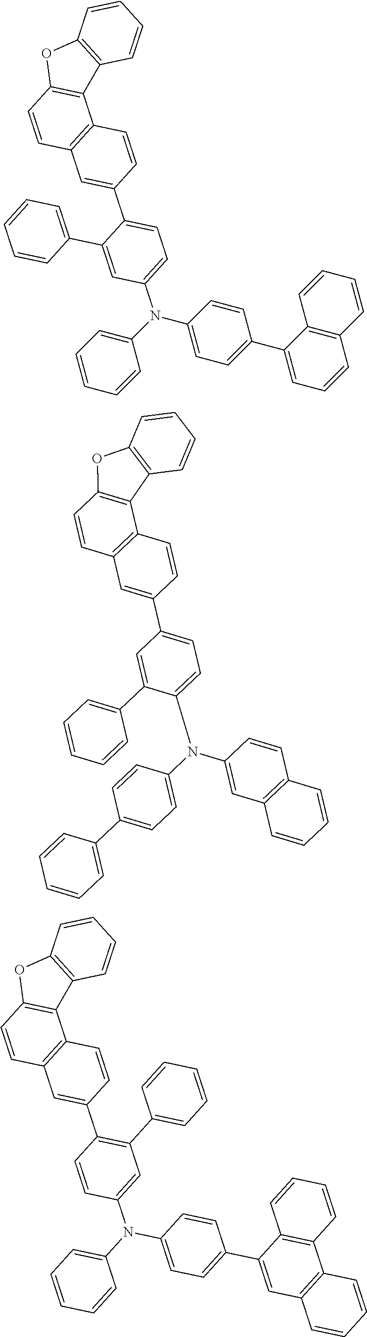 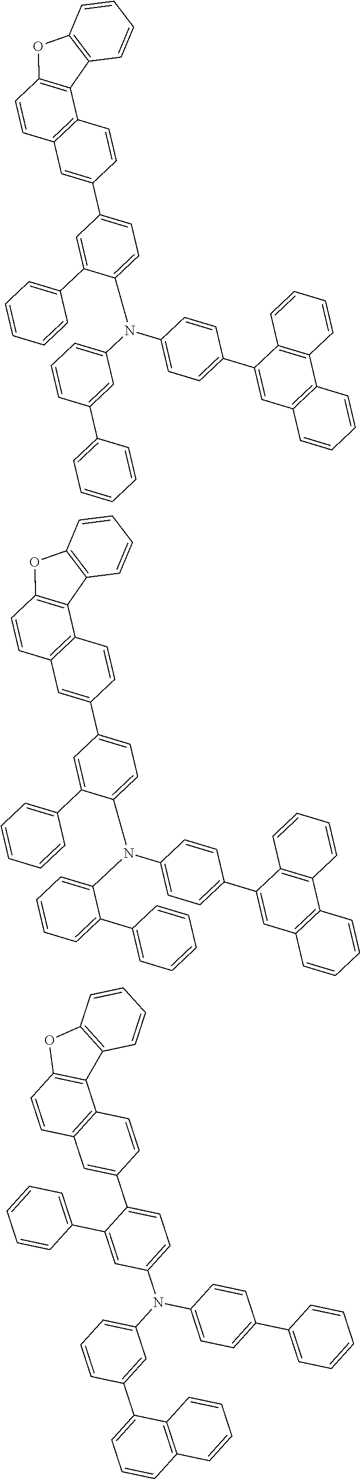 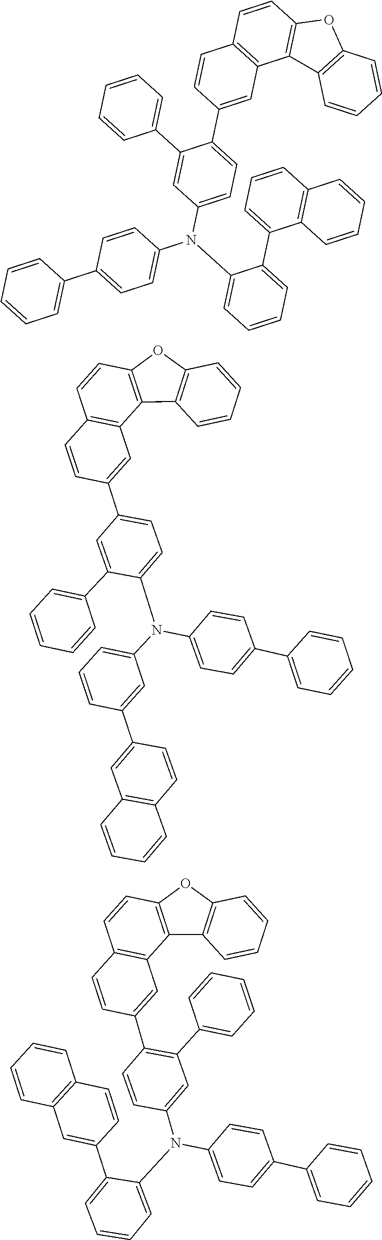 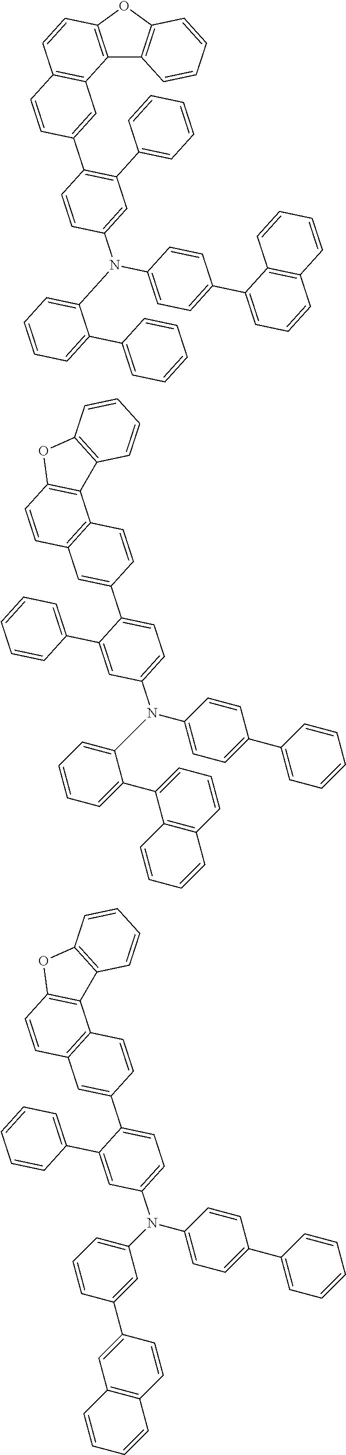 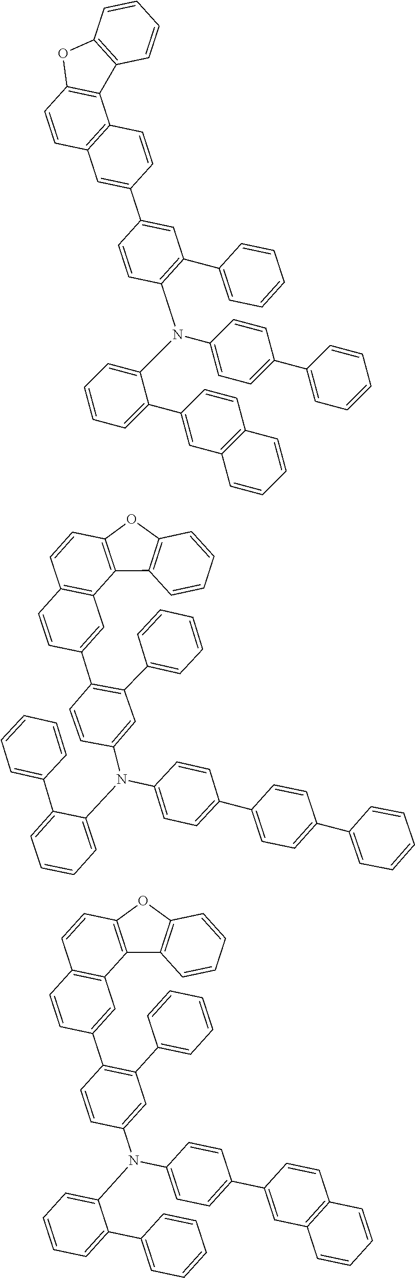 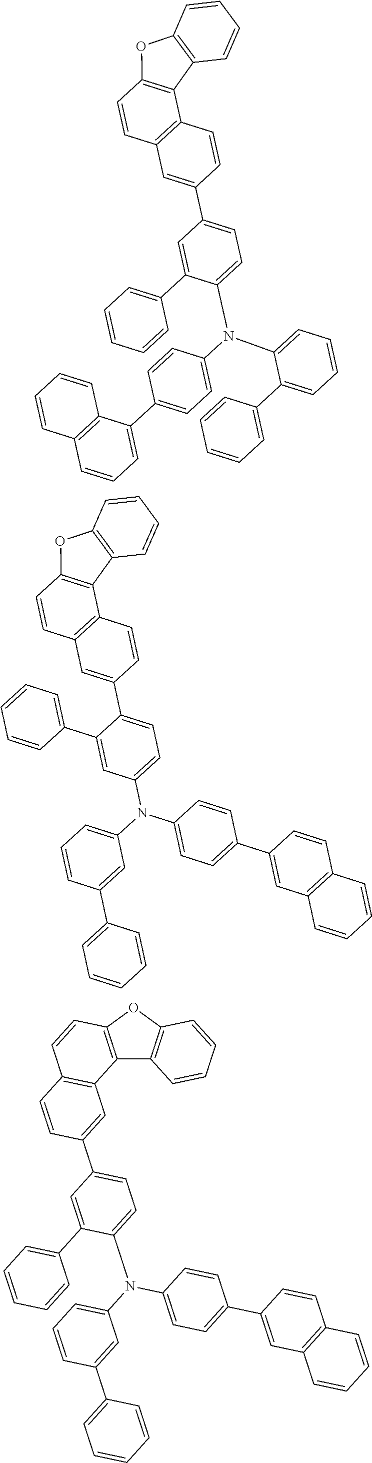 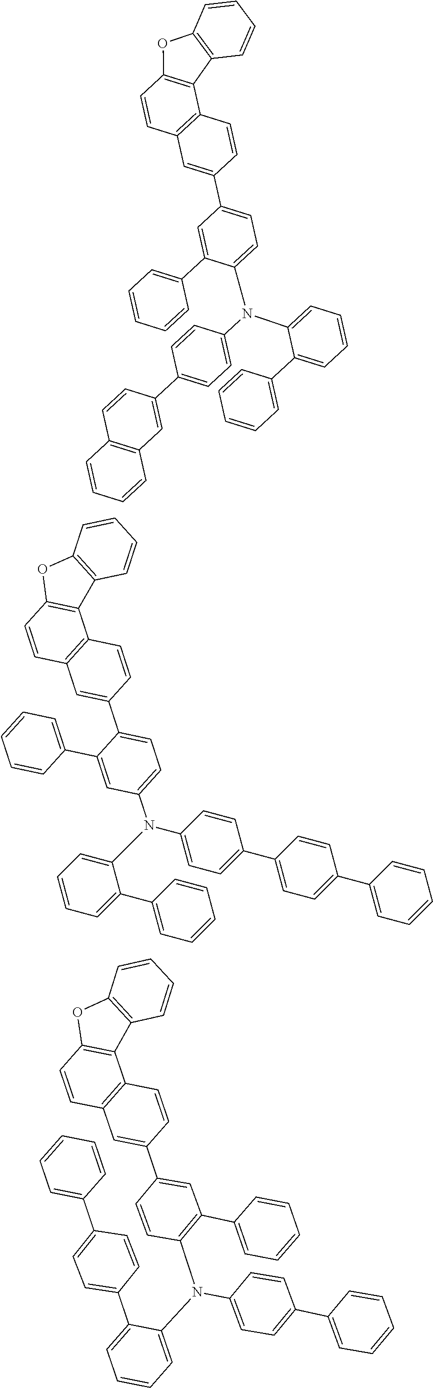 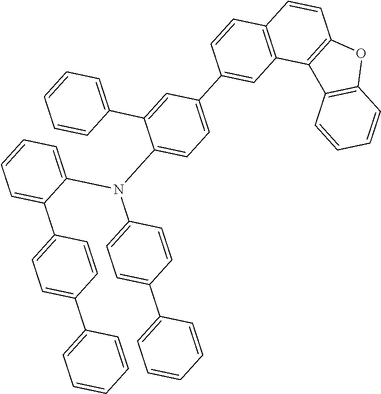 [SMILES: c1ccc(-c2ccc(-c3ccccc3N(c3ccc(-c4ccccc4)cc3)c3ccc(-c4ccc5ccc6oc7ccccc7c6c5c4)cc3-c3ccccc3)cc2)cc1] 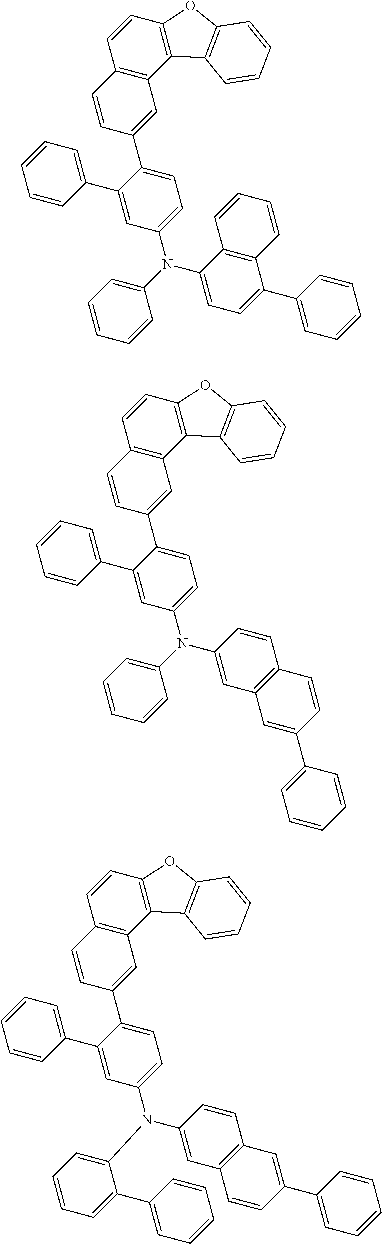 [SMILES: c1ccc(-c2cc(N(c3ccccc3)c3ccc(-c4ccccc4)c4ccccc34)ccc2-c2ccc3ccc4oc5ccccc5c4c3c2)cc1 ; c1ccc(-c2ccc3ccc(N(c4ccccc4)c4ccc(-c5ccc6ccc7oc8ccccc8c7c6c5)c(-c5ccccc5)c4)cc3c2)cc1 ; c1ccc(-c2ccc3cc(N(c4ccc(-c5ccc6ccc7oc8ccccc8c7c6c5)c(-c5ccccc5)c4)c4ccccc4-c4ccccc4)ccc3c2)cc1] 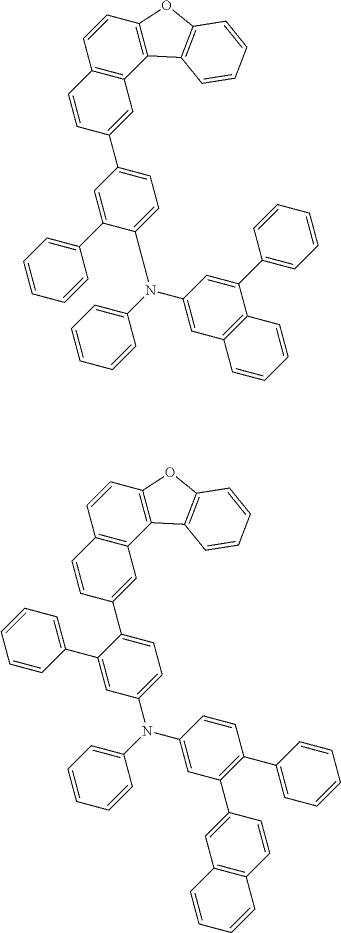 [SMILES: c1ccc(-c2cc(-c3ccc4ccc5oc6ccccc6c5c4c3)ccc2N(c2ccccc2)c2cc(-c3ccccc3)c3ccccc3c2)cc1 ; c1ccc(-c2ccc(N(c3ccccc3)c3ccc(-c4ccc5ccc6oc7ccccc7c6c5c4)c(-c4ccccc4)c3)cc2-c2ccc3ccccc3c2)cc1] 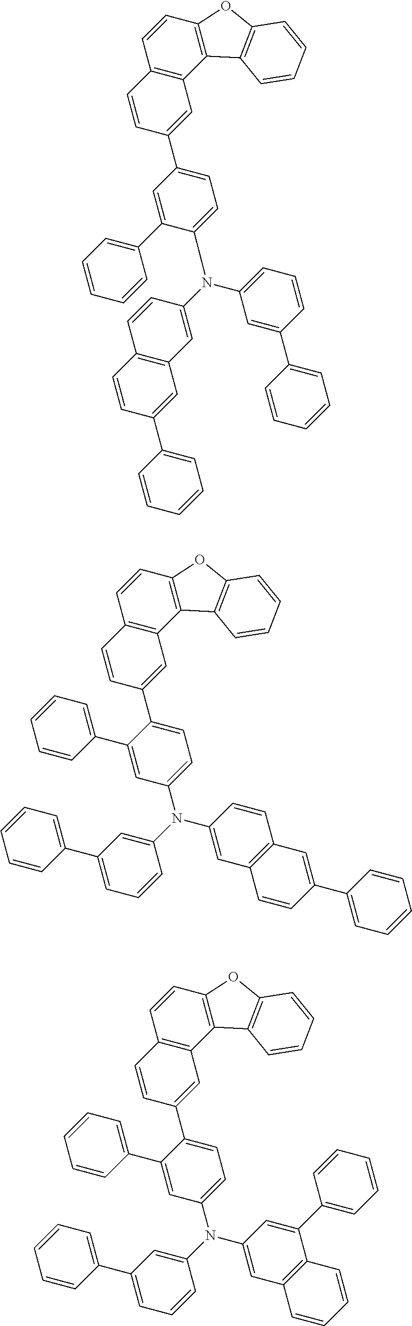 [SMILES: c1ccc(-c2cccc(N(c3ccc4ccc(-c5ccccc5)cc4c3)c3ccc(-c4ccc5ccc6oc7ccccc7c6c5c4)cc3-c3ccccc3)c2)cc1 ; c1ccc(-c2cccc(N(c3ccc(-c4ccc5ccc6oc7ccccc7c6c5c4)c(-c4ccccc4)c3)c3ccc4cc(-c5ccccc5)ccc4c3)c2)cc1 ; c1ccc(-c2cccc(N(c3ccc(-c4ccc5ccc6oc7ccccc7c6c5c4)c(-c4ccccc4)c3)c3cc(-c4ccccc4)c4ccccc4c3)c2)cc1] 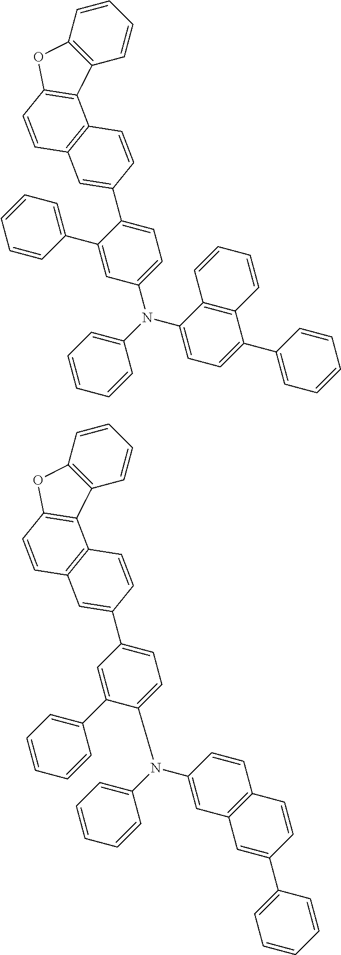 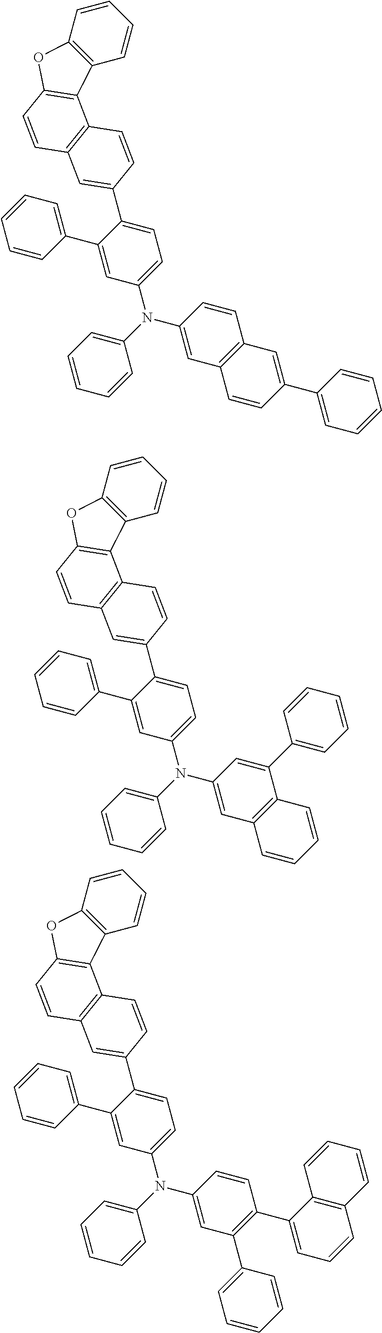 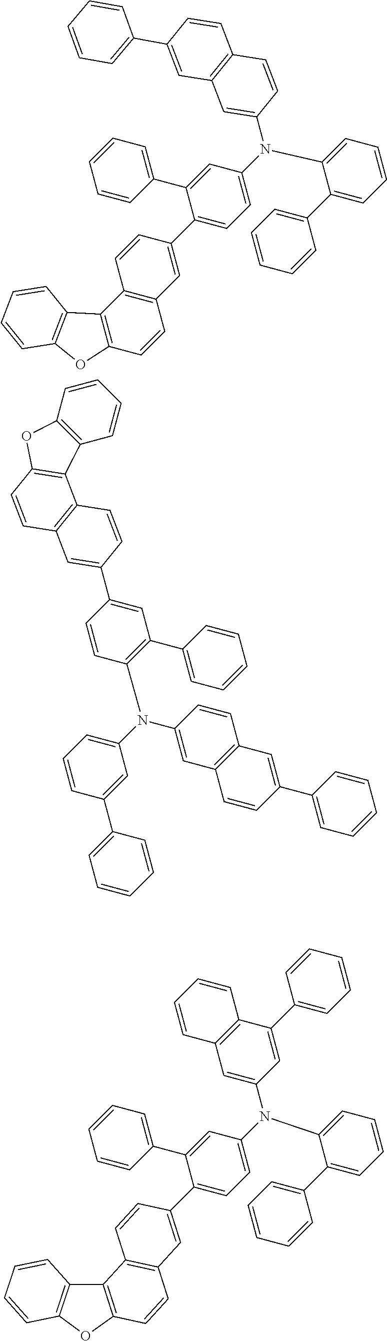 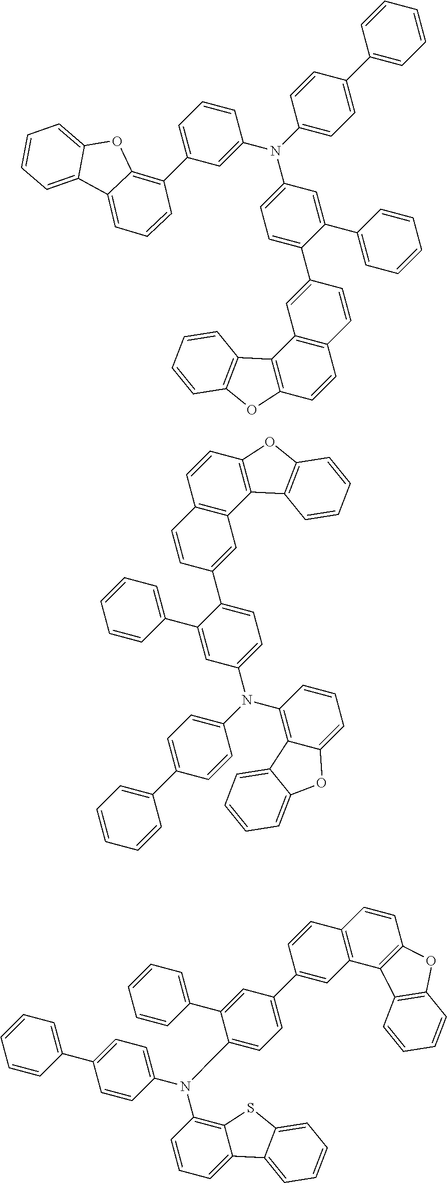 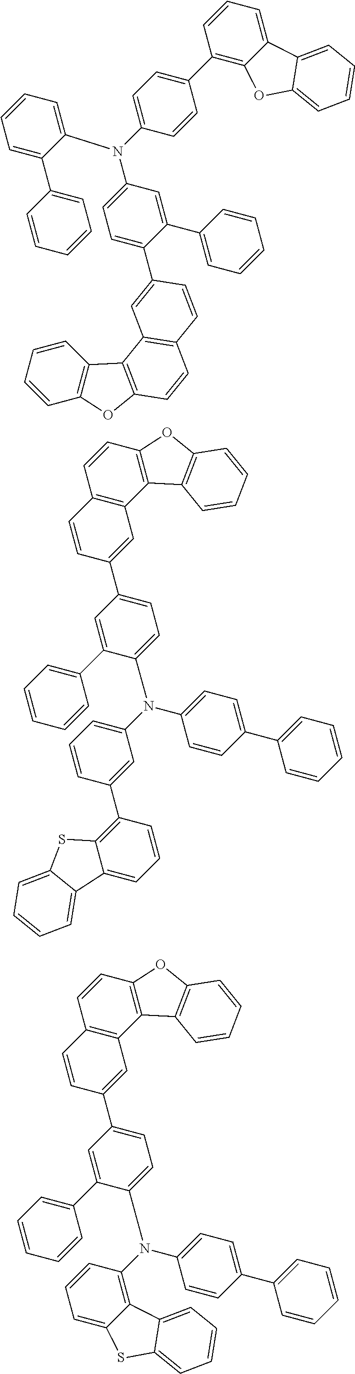 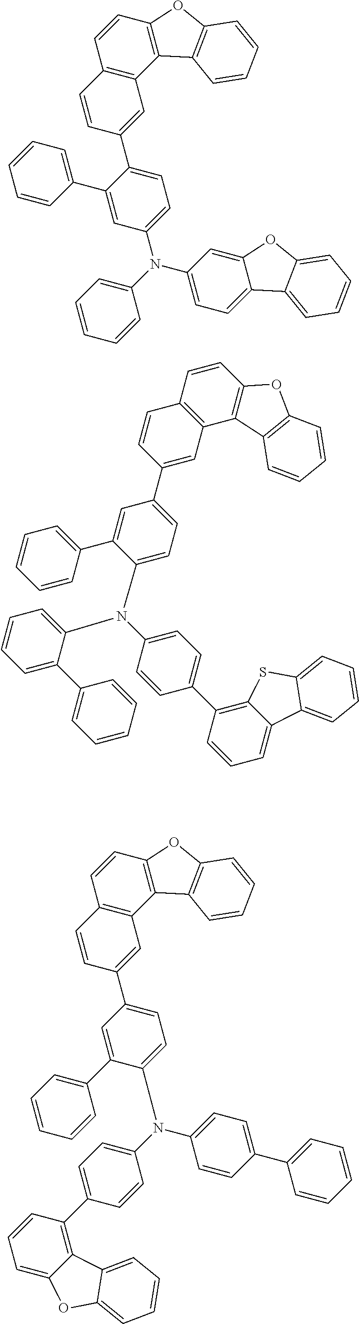 [SMILES: c1ccc(-c2cc(N(c3ccccc3)c3ccc4c(c3)oc3ccccc34)ccc2-c2ccc3ccc4oc5ccccc5c4c3c2)cc1 ; c1ccc(-c2ccccc2N(c2ccc(-c3cccc4c3sc3ccccc34)cc2)c2ccc(-c3ccc4ccc5oc6ccccc6c5c4c3)cc2-c2ccccc2)cc1 ; c1ccc(-c2ccc(N(c3ccc(-c4cccc5oc6ccccc6c45)cc3)c3ccc(-c4ccc5ccc6oc7ccccc7c6c5c4)cc3-c3ccccc3)cc2)cc1] 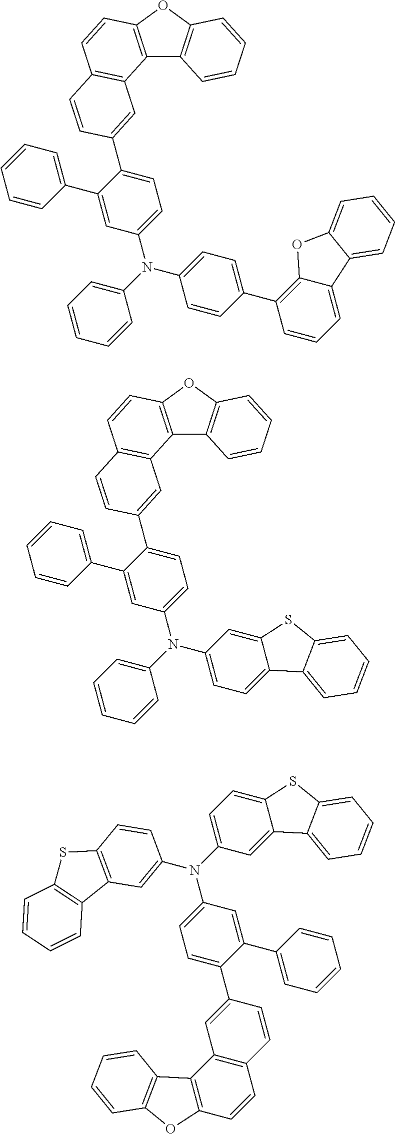 [SMILES: c1ccc(-c2cc(N(c3ccccc3)c3ccc(-c4cccc5c4oc4ccccc45)cc3)ccc2-c2ccc3ccc4oc5ccccc5c4c3c2)cc1 ; c1ccc(-c2cc(N(c3ccccc3)c3ccc4c(c3)sc3ccccc34)ccc2-c2ccc3ccc4oc5ccccc5c4c3c2)cc1 ; c1ccc(-c2cc(N(c3ccc4sc5ccccc5c4c3)c3ccc4sc5ccccc5c4c3)ccc2-c2ccc3ccc4oc5ccccc5c4c3c2)cc1] 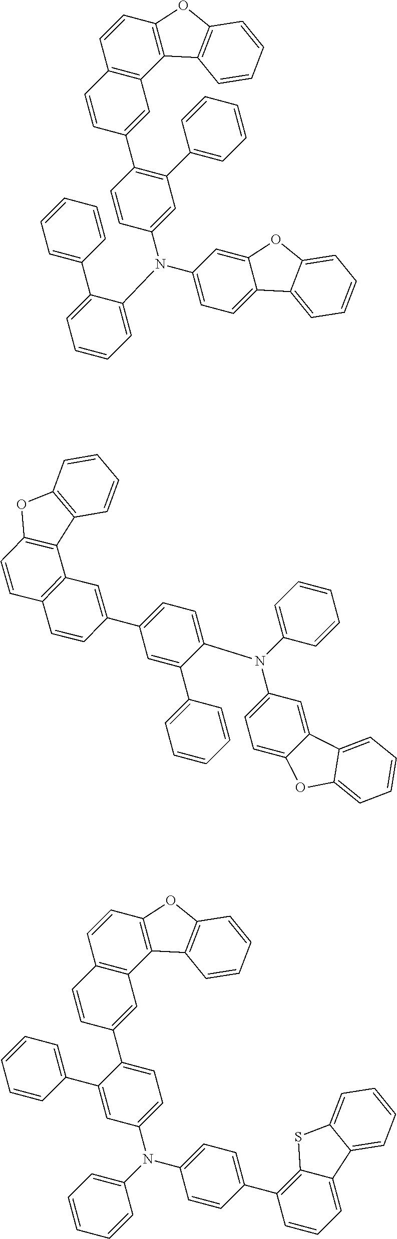 [SMILES: c1ccc(-c2cc(N(c3ccc4c(c3)oc3ccccc34)c3ccccc3-c3ccccc3)ccc2-c2ccc3ccc4oc5ccccc5c4c3c2)cc1 ; c1ccc(-c2cc(-c3ccc4ccc5oc6ccccc6c5c4c3)ccc2N(c2ccccc2)c2ccc3oc4ccccc4c3c2)cc1 ; c1ccc(-c2cc(N(c3ccccc3)c3ccc(-c4cccc5c4sc4ccccc45)cc3)ccc2-c2ccc3ccc4oc5ccccc5c4c3c2)cc1] 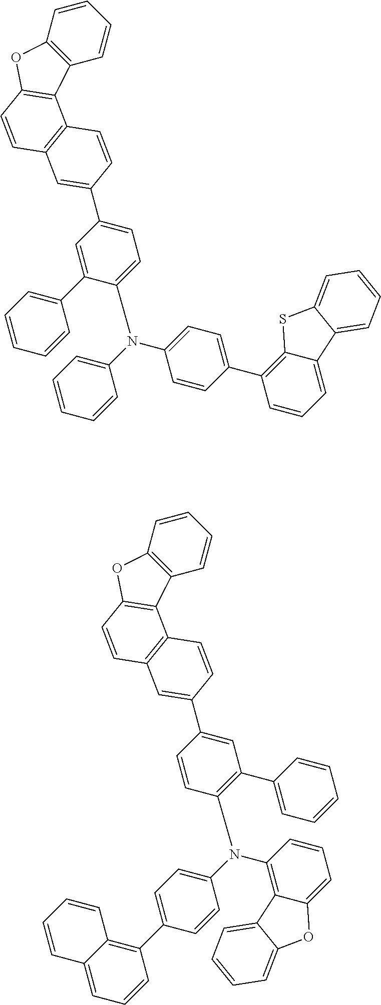 [SMILES: c1ccc(-c2cc(-c3ccc4c(ccc5oc6ccccc6c54)c3)ccc2N(c2ccccc2)c2ccc(-c3cccc4c3sc3ccccc34)cc2)cc1 ; c1ccc(-c2cc(-c3ccc4c(ccc5oc6ccccc6c54)c3)ccc2N(c2ccc(-c3cccc4ccccc34)cc2)c2cccc3oc4ccccc4c23)cc1] 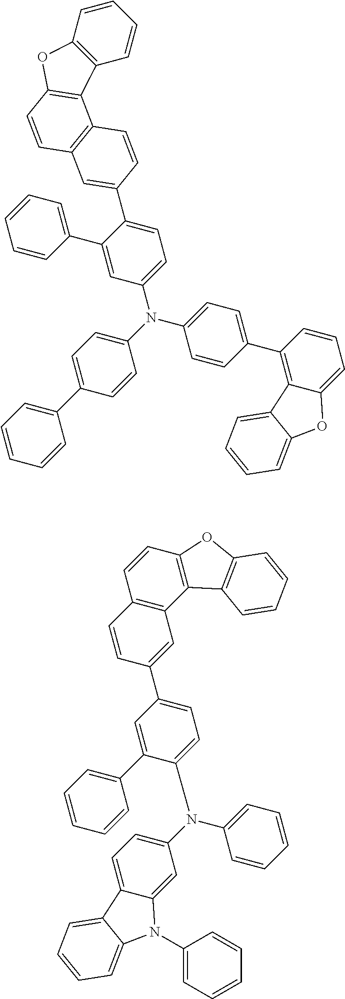 [SMILES: c1ccc(-c2ccc(N(c3ccc(-c4cccc5oc6ccccc6c45)cc3)c3ccc(-c4ccc5c(ccc6oc7ccccc7c65)c4)c(-c4ccccc4)c3)cc2)cc1 ; c1ccc(-c2cc(-c3ccc4ccc5oc6ccccc6c5c4c3)ccc2N(c2ccccc2)c2ccc3c4ccccc4n(-c4ccccc4)c3c2)cc1] 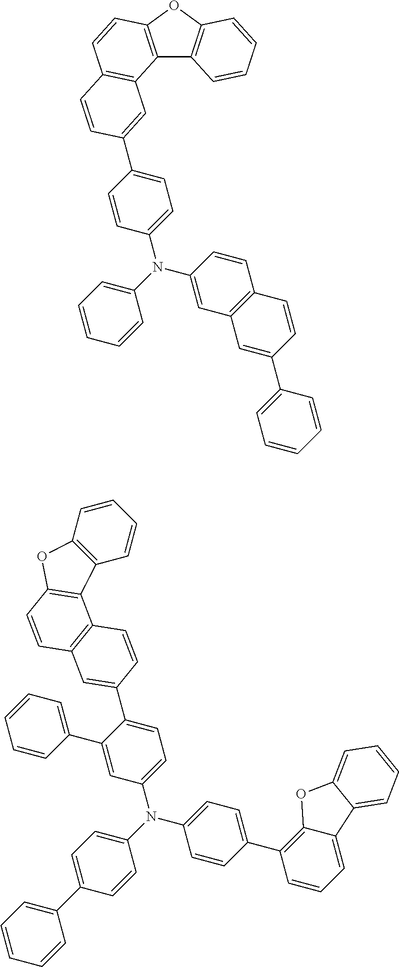 [SMILES: c1ccc(-c2ccc3ccc(N(c4ccccc4)c4ccc(-c5ccc6ccc7oc8ccccc8c7c6c5)cc4)cc3c2)cc1 ; c1ccc(-c2ccc(N(c3ccc(-c4cccc5c4oc4ccccc45)cc3)c3ccc(-c4ccc5c(ccc6oc7ccccc7c65)c4)c(-c4ccccc4)c3)cc2)cc1] 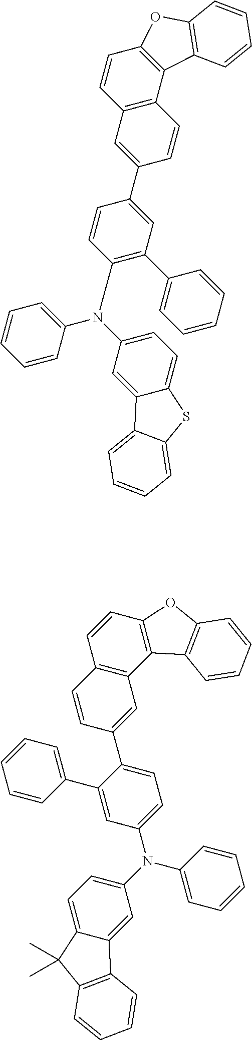 [SMILES: c1ccc(-c2cc(-c3ccc4c(ccc5oc6ccccc6c54)c3)ccc2N(c2ccccc2)c2ccc3sc4ccccc4c3c2)cc1 ; CC1(C)c2ccccc2-c2cc(N(c3ccccc3)c3ccc(-c4ccc5ccc6oc7ccccc7c6c5c4)c(-c4ccccc4)c3)ccc21] 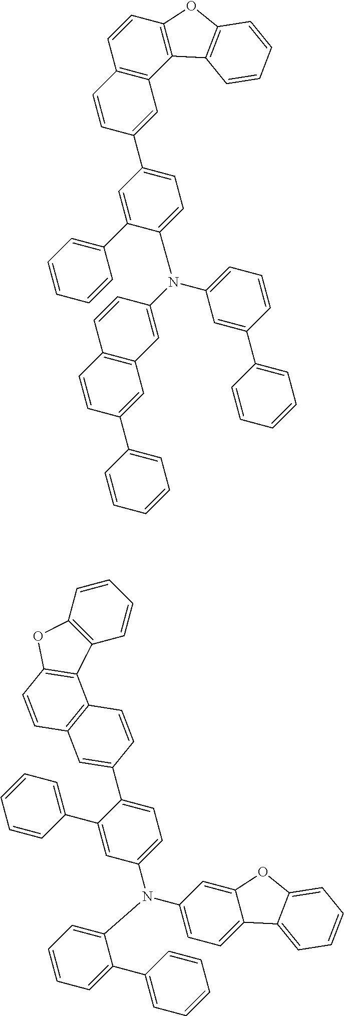 [SMILES: c1ccc(-c2cccc(N(c3ccc4ccc(-c5ccccc5)cc4c3)c3ccc(-c4ccc5ccc6oc7ccccc7c6c5c4)cc3-c3ccccc3)c2)cc1 ; c1ccc(-c2cc(N(c3ccc4c(c3)oc3ccccc34)c3ccccc3-c3ccccc3)ccc2-c2ccc3c(ccc4oc5ccccc5c43)c2)cc1] 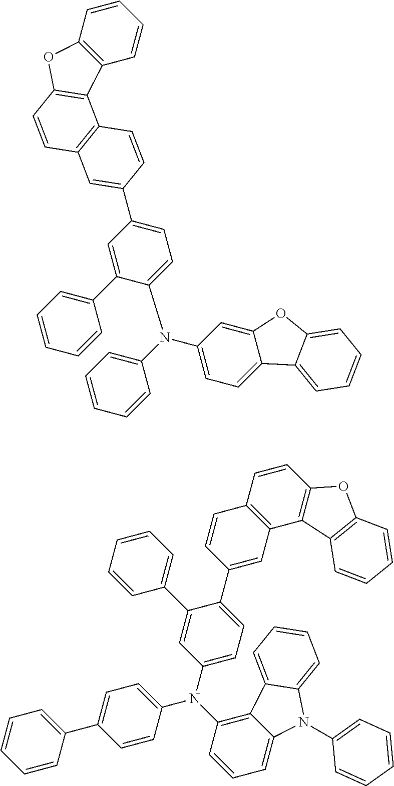 [SMILES: c1ccc(-c2cc(-c3ccc4c(ccc5oc6ccccc6c54)c3)ccc2N(c2ccccc2)c2ccc3c(c2)oc2ccccc23)cc1 ; c1ccc(-c2ccc(N(c3ccc(-c4ccc5ccc6oc7ccccc7c6c5c4)c(-c4ccccc4)c3)c3cccc4c3c3ccccc3n4-c3ccccc3)cc2)cc1] 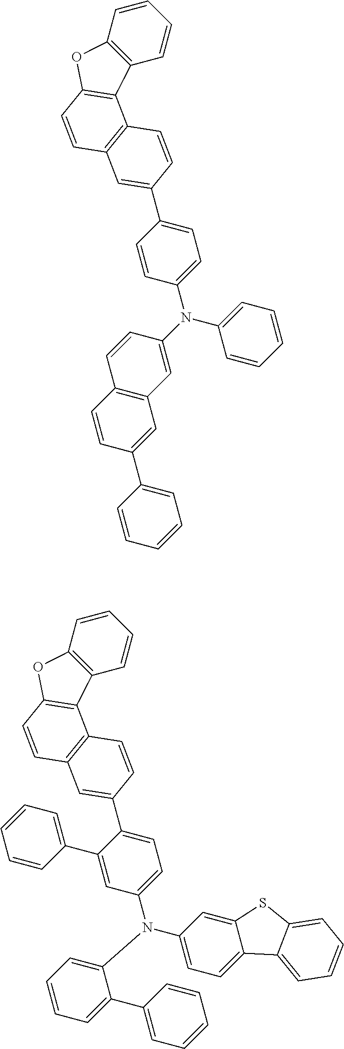 [SMILES: c1ccc(-c2ccc3ccc(N(c4ccccc4)c4ccc(-c5ccc6c(ccc7oc8ccccc8c76)c5)cc4)cc3c2)cc1 ; c1ccc(-c2cc(N(c3ccc4c(c3)sc3ccccc34)c3ccccc3-c3ccccc3)ccc2-c2ccc3c(ccc4oc5ccccc5c43)c2)cc1] 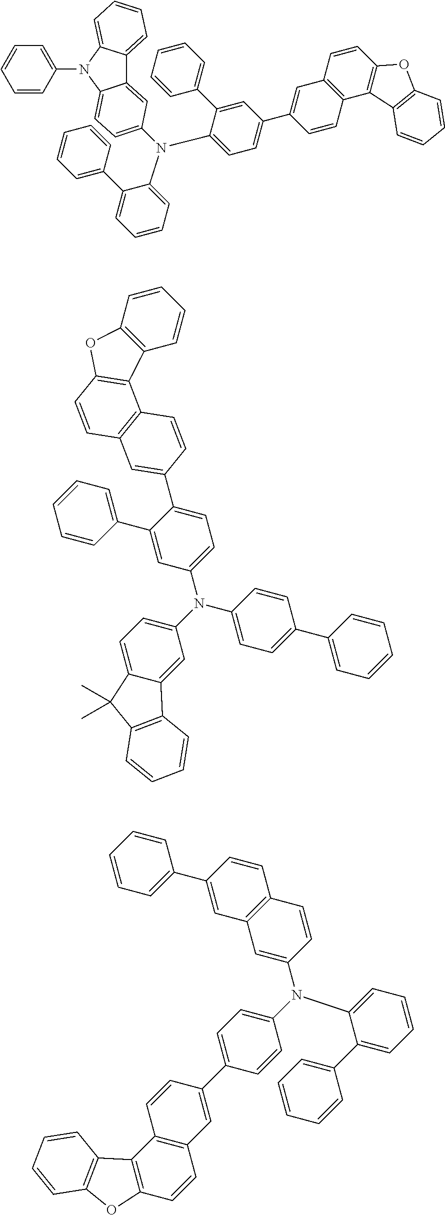 [SMILES: c1ccc(-c2ccccc2N(c2ccc3c(c2)c2ccccc2n3-c2ccccc2)c2ccc(-c3ccc4c(ccc5oc6ccccc6c54)c3)cc2-c2ccccc2)cc1 ; CC1(C)c2ccccc2-c2cc(N(c3ccc(-c4ccccc4)cc3)c3ccc(-c4ccc5c(ccc6oc7ccccc7c65)c4)c(-c4ccccc4)c3)ccc21 ; c1ccc(-c2ccc3ccc(N(c4ccc(-c5ccc6c(ccc7oc8ccccc8c76)c5)cc4)c4ccccc4-c4ccccc4)cc3c2)cc1] 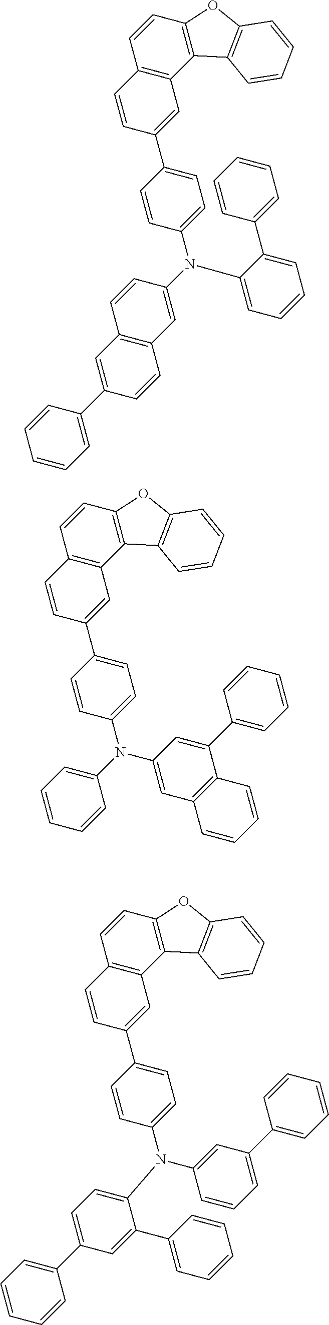 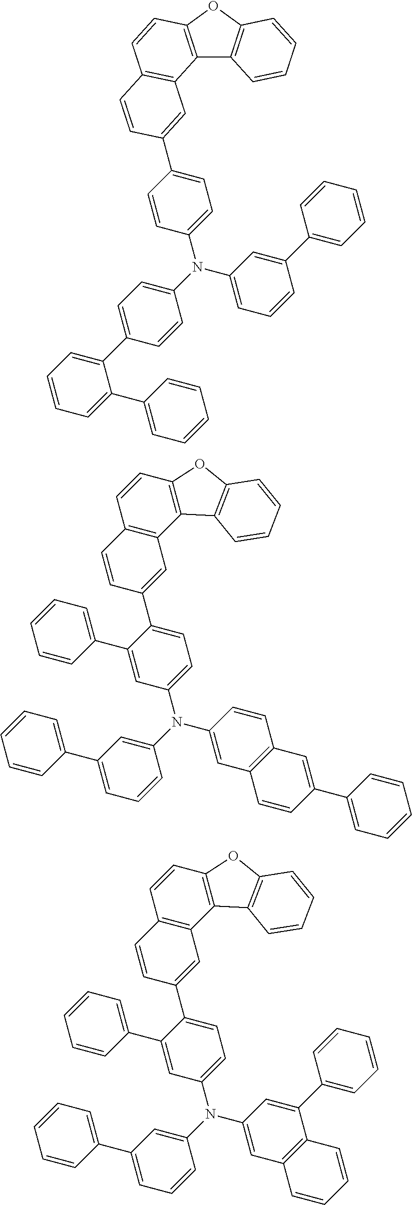 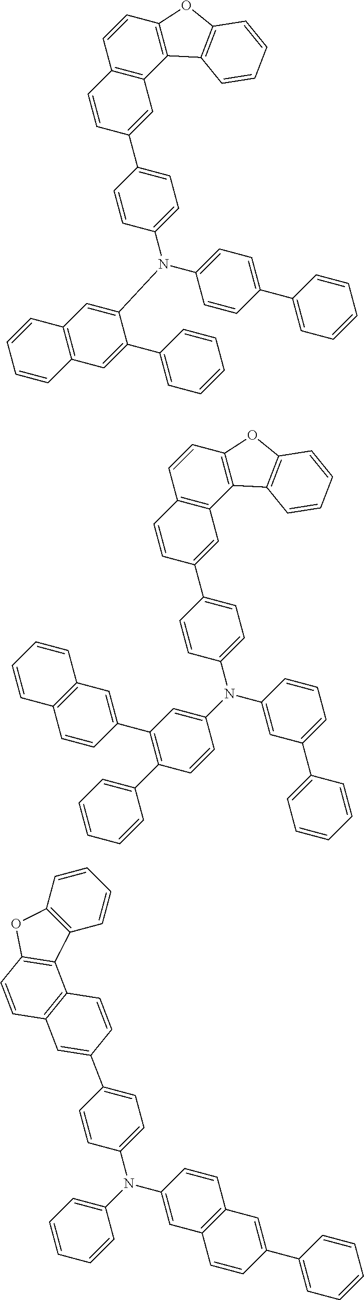 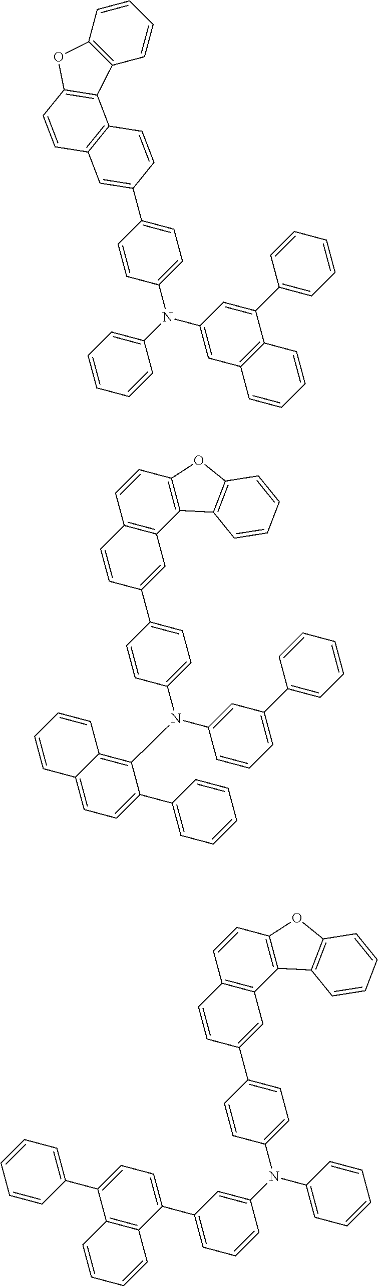 [SMILES: c1ccc(-c2cc(N(c3ccccc3)c3ccc(-c4ccc5c(ccc6oc7ccccc7c65)c4)cc3)cc3ccccc23)cc1 ; c1ccc(-c2cccc(N(c3ccc(-c4ccc5ccc6oc7ccccc7c6c5c4)cc3)c3c(-c4ccccc4)ccc4ccccc34)c2)cc1 ; c1ccc(-c2ccc(-c3cccc(N(c4ccccc4)c4ccc(-c5ccc6ccc7oc8ccccc8c7c6c5)cc4)c3)c3ccccc23)cc1] 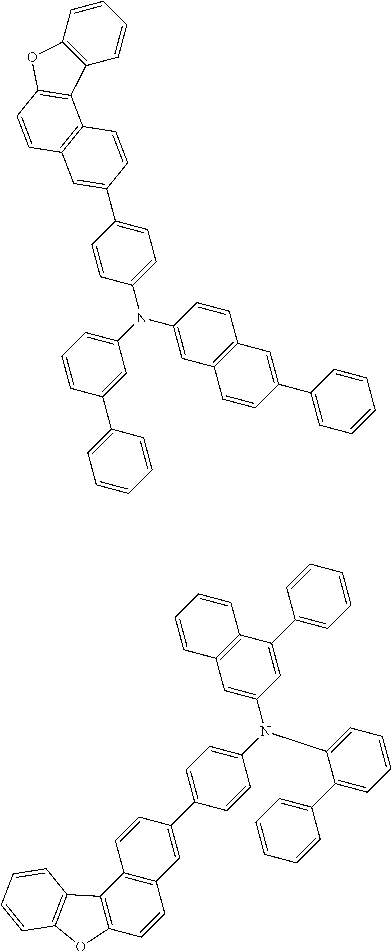 [SMILES: c1ccc(-c2cccc(N(c3ccc(-c4ccc5c(ccc6oc7ccccc7c65)c4)cc3)c3ccc4cc(-c5ccccc5)ccc4c3)c2)cc1 ; c1ccc(-c2ccccc2N(c2ccc(-c3ccc4c(ccc5oc6ccccc6c54)c3)cc2)c2cc(-c3ccccc3)c3ccccc3c2)cc1] 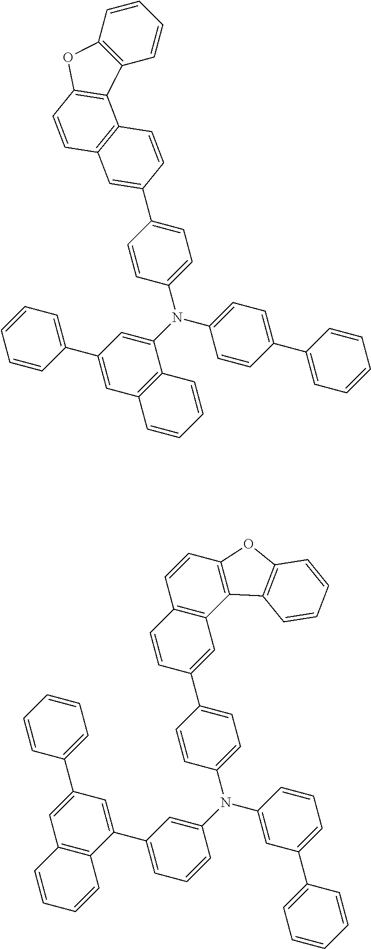 [SMILES: c1ccc(-c2ccc(N(c3ccc(-c4ccc5c(ccc6oc7ccccc7c65)c4)cc3)c3cc(-c4ccccc4)cc4ccccc34)cc2)cc1 ; c1ccc(-c2cccc(N(c3ccc(-c4ccc5ccc6oc7ccccc7c6c5c4)cc3)c3cccc(-c4cc(-c5ccccc5)cc5ccccc45)c3)c2)cc1] 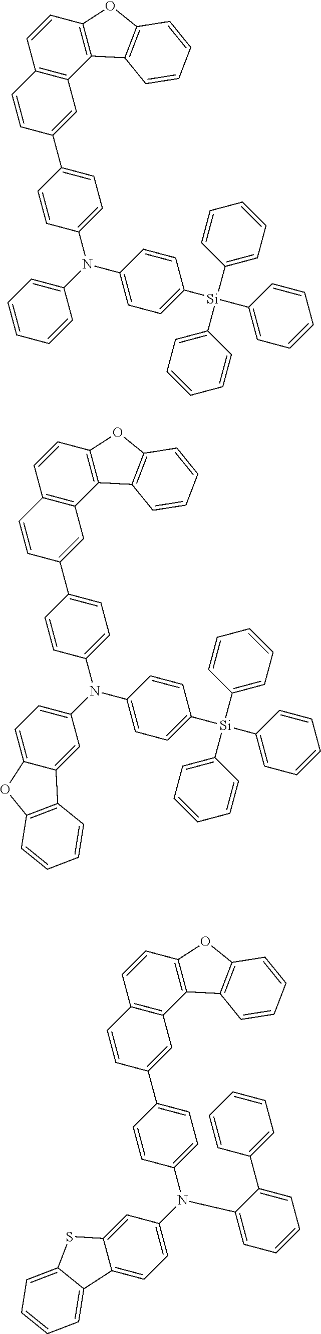 [SMILES: c1ccc(N(c2ccc(-c3ccc4ccc5oc6ccccc6c5c4c3)cc2)c2ccc([Si](c3ccccc3)(c3ccccc3)c3ccccc3)cc2)cc1 ; c1ccc([Si](c2ccccc2)(c2ccccc2)c2ccc(N(c3ccc(-c4ccc5ccc6oc7ccccc7c6c5c4)cc3)c3ccc4oc5ccccc5c4c3)cc2)cc1 ; c1ccc(-c2ccccc2N(c2ccc(-c3ccc4ccc5oc6ccccc6c5c4c3)cc2)c2ccc3c(c2)sc2ccccc23)cc1] 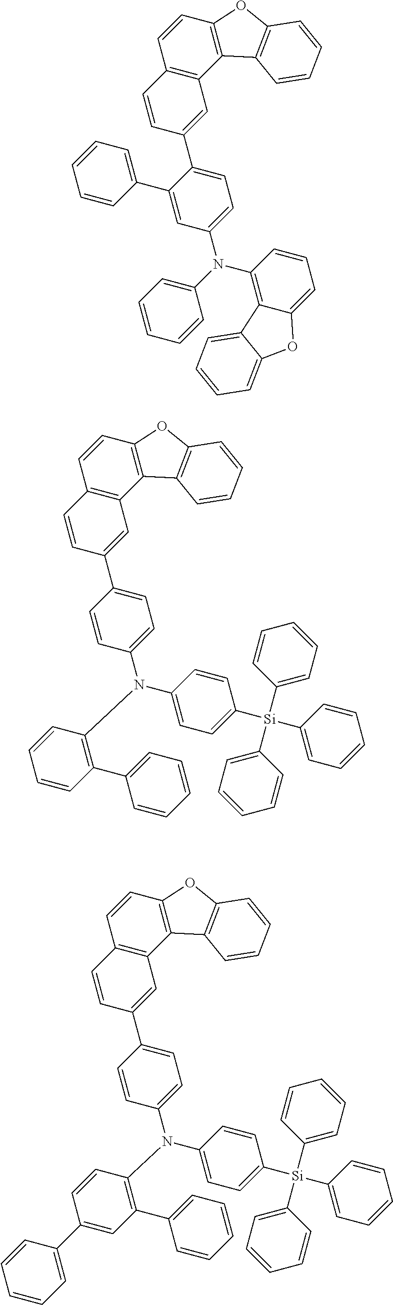 [SMILES: c1ccc(-c2cc(N(c3ccccc3)c3cccc4oc5ccccc5c34)ccc2-c2ccc3ccc4oc5ccccc5c4c3c2)cc1 ; c1ccc(-c2ccccc2N(c2ccc(-c3ccc4ccc5oc6ccccc6c5c4c3)cc2)c2ccc([Si](c3ccccc3)(c3ccccc3)c3ccccc3)cc2)cc1 ; c1ccc(-c2ccc(N(c3ccc(-c4ccc5ccc6oc7ccccc7c6c5c4)cc3)c3ccc([Si](c4ccccc4)(c4ccccc4)c4ccccc4)cc3)c(-c3ccccc3)c2)cc1] 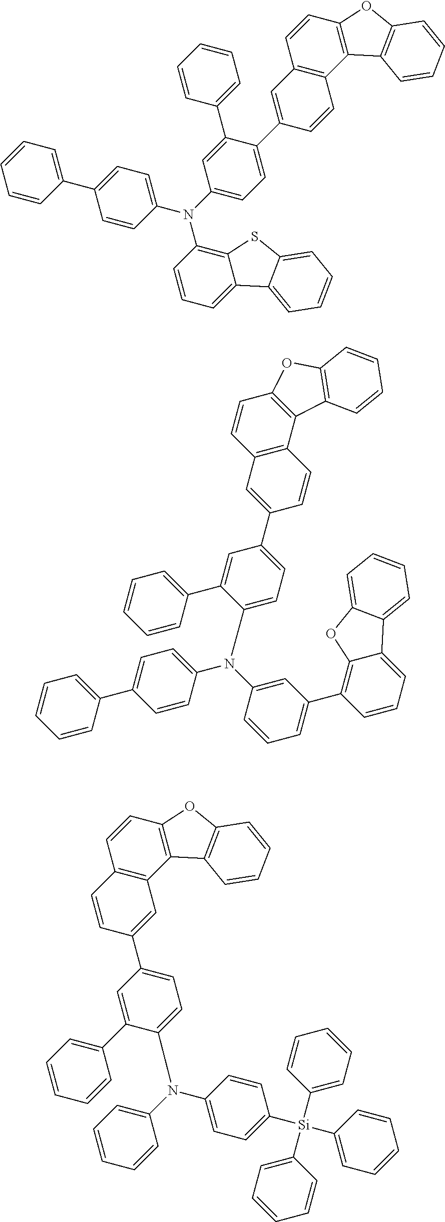 [SMILES: c1ccc(-c2ccc(N(c3ccc(-c4ccc5c(ccc6oc7ccccc7c65)c4)c(-c4ccccc4)c3)c3cccc4c3sc3ccccc34)cc2)cc1 ; c1ccc(-c2ccc(N(c3cccc(-c4cccc5c4oc4ccccc45)c3)c3ccc(-c4ccc5c(ccc6oc7ccccc7c65)c4)cc3-c3ccccc3)cc2)cc1 ; c1ccc(-c2cc(-c3ccc4ccc5oc6ccccc6c5c4c3)ccc2N(c2ccccc2)c2ccc([Si](c3ccccc3)(c3ccccc3)c3ccccc3)cc2)cc1] 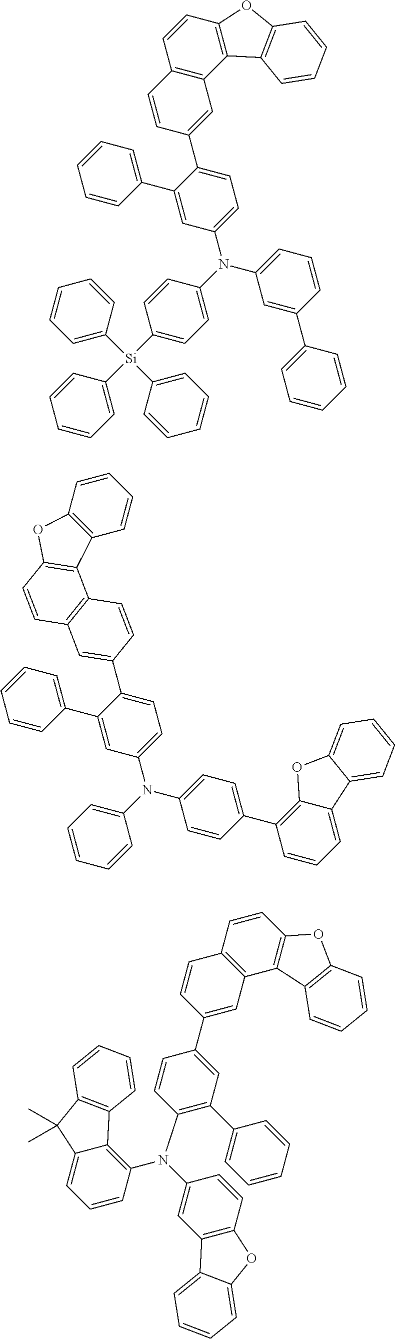 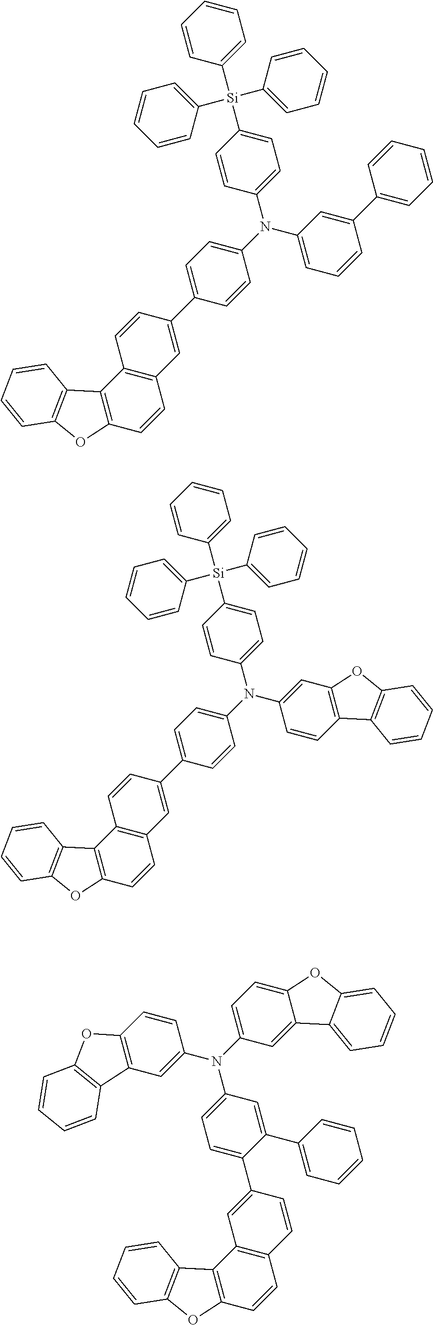 [SMILES: c1ccc(-c2cccc(N(c3ccc(-c4ccc5c(ccc6oc7ccccc7c65)c4)cc3)c3ccc([Si](c4ccccc4)(c4ccccc4)c4ccccc4)cc3)c2)cc1 ; c1ccc([Si](c2ccccc2)(c2ccccc2)c2ccc(N(c3ccc(-c4ccc5c(ccc6oc7ccccc7c65)c4)cc3)c3ccc4c(c3)oc3ccccc34)cc2)cc1 ; c1ccc(-c2cc(N(c3ccc4oc5ccccc5c4c3)c3ccc4oc5ccccc5c4c3)ccc2-c2ccc3ccc4oc5ccccc5c4c3c2)cc1] 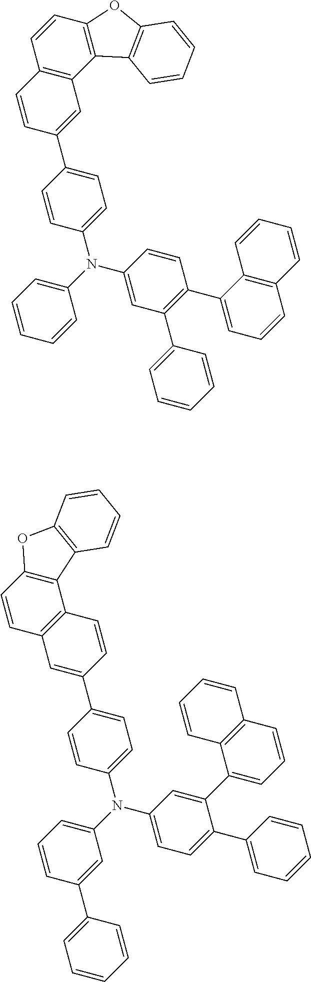 [SMILES: c1ccc(-c2cc(N(c3ccccc3)c3ccc(-c4ccc5ccc6oc7ccccc7c6c5c4)cc3)ccc2-c2cccc3ccccc23)cc1 ; c1ccc(-c2cccc(N(c3ccc(-c4ccc5c(ccc6oc7ccccc7c65)c4)cc3)c3ccc(-c4ccccc4)c(-c4cccc5ccccc45)c3)c2)cc1] 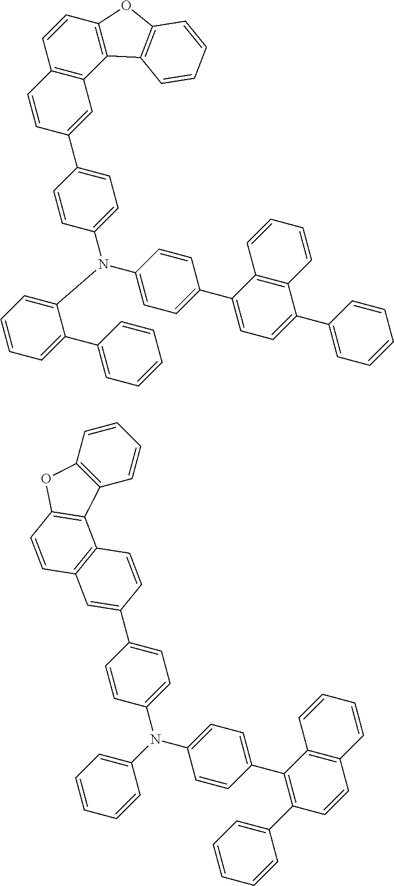 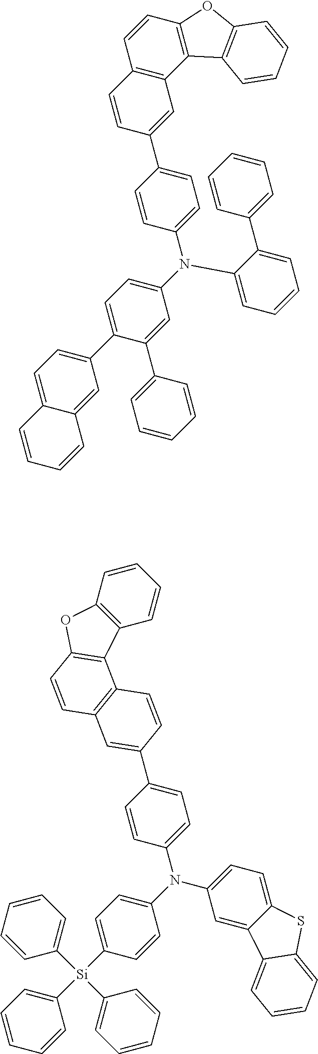 [SMILES: c1ccc(-c2cc(N(c3ccc(-c4ccc5ccc6oc7ccccc7c6c5c4)cc3)c3ccccc3-c3ccccc3)ccc2-c2ccc3ccccc3c2)cc1 ; c1ccc([Si](c2ccccc2)(c2ccccc2)c2ccc(N(c3ccc(-c4ccc5c(ccc6oc7ccccc7c65)c4)cc3)c3ccc4sc5ccccc5c4c3)cc2)cc1] 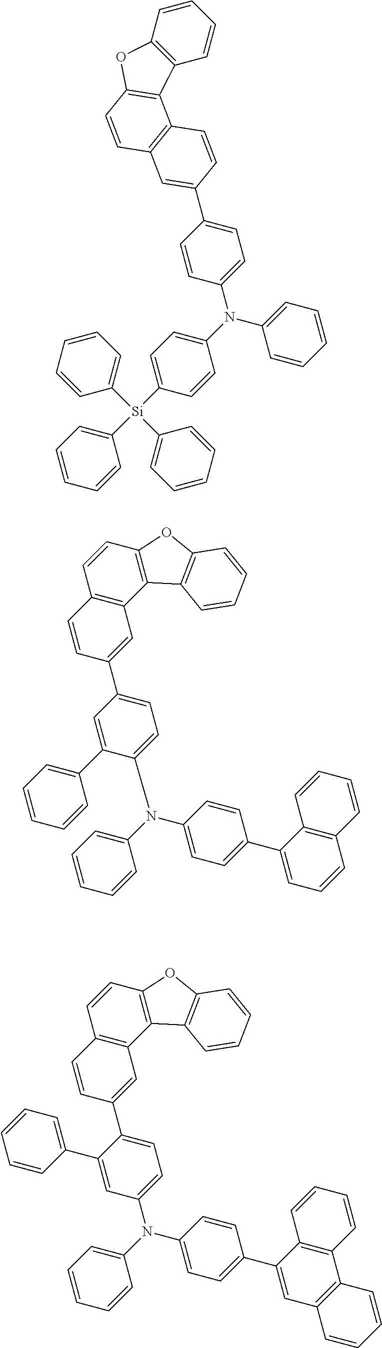 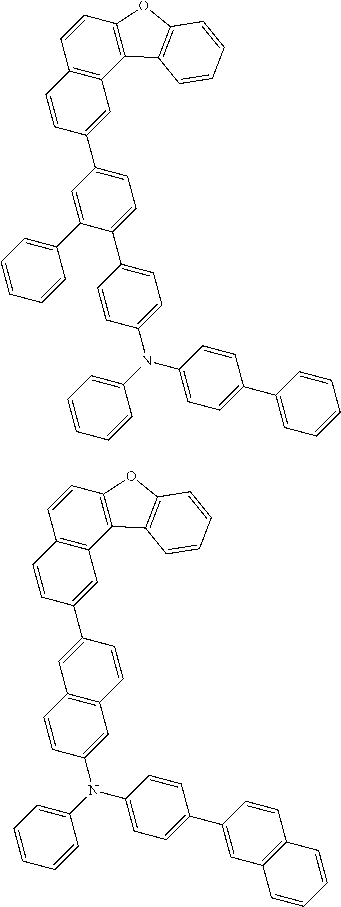 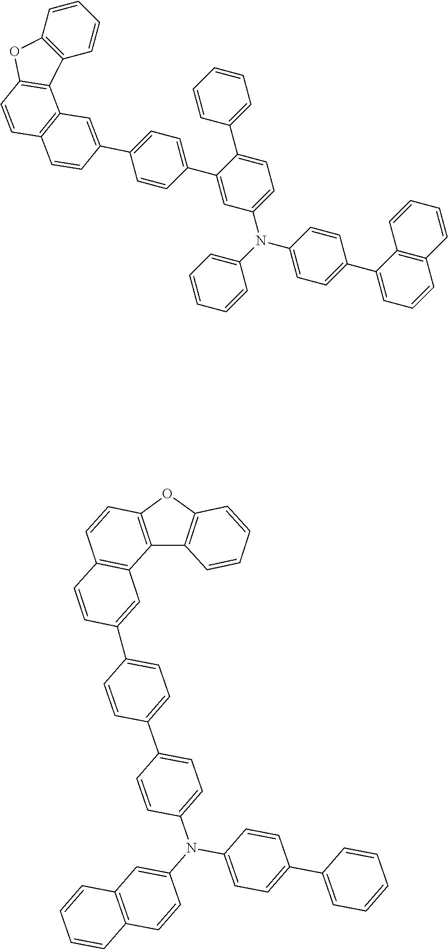 [SMILES: c1ccc(-c2ccc(N(c3ccccc3)c3ccc(-c4cccc5ccccc45)cc3)cc2-c2ccc(-c3ccc4ccc5oc6ccccc6c5c4c3)cc2)cc1 ; c1ccc(-c2ccc(N(c3ccc(-c4ccc(-c5ccc6ccc7oc8ccccc8c7c6c5)cc4)cc3)c3ccc4ccccc4c3)cc2)cc1] 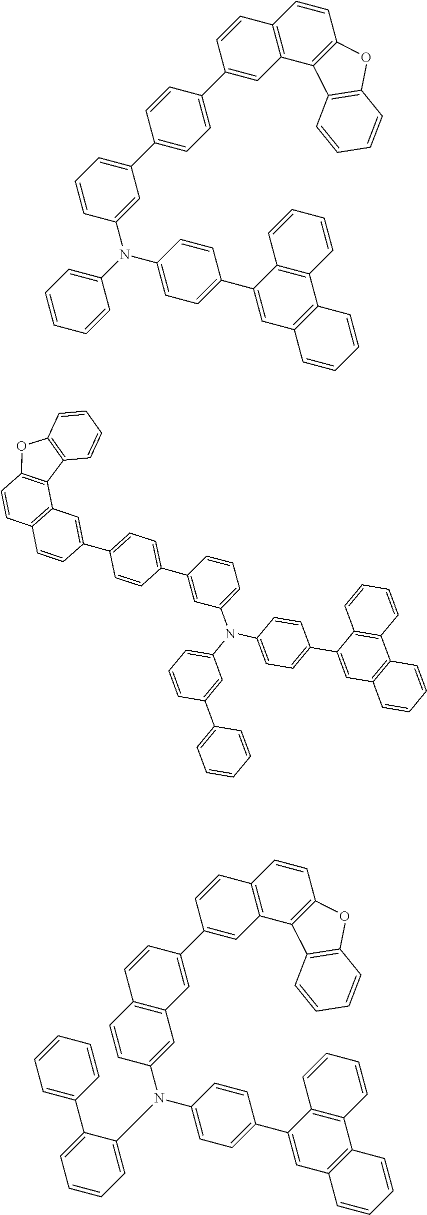 [SMILES: c1ccc(N(c2ccc(-c3cc4ccccc4c4ccccc34)cc2)c2cccc(-c3ccc(-c4ccc5ccc6oc7ccccc7c6c5c4)cc3)c2)cc1 ; c1ccc(-c2cccc(N(c3ccc(-c4cc5ccccc5c5ccccc45)cc3)c3cccc(-c4ccc(-c5ccc6ccc7oc8ccccc8c7c6c5)cc4)c3)c2)cc1 ; c1ccc(-c2ccccc2N(c2ccc(-c3cc4ccccc4c4ccccc34)cc2)c2ccc3ccc(-c4ccc5ccc6oc7ccccc7c6c5c4)cc3c2)cc1] 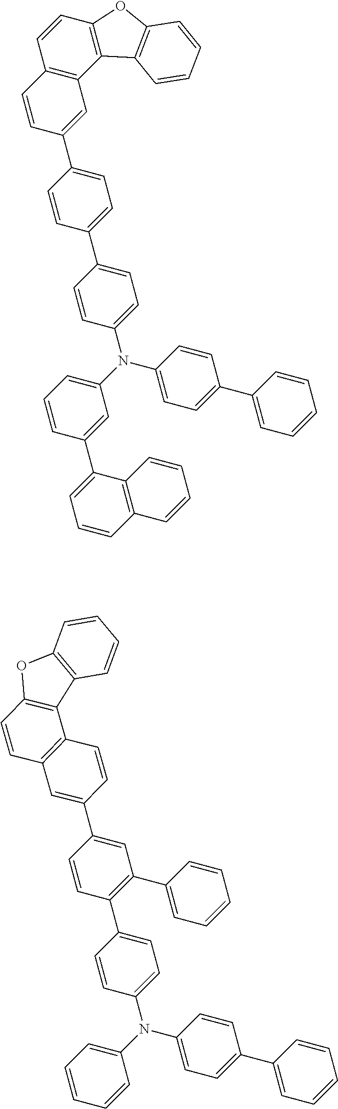 [SMILES: c1ccc(-c2ccc(N(c3ccc(-c4ccc(-c5ccc6ccc7oc8ccccc8c7c6c5)cc4)cc3)c3cccc(-c4cccc5ccccc45)c3)cc2)cc1 ; c1ccc(-c2ccc(N(c3ccccc3)c3ccc(-c4ccc(-c5ccc6c(ccc7oc8ccccc8c76)c5)cc4-c4ccccc4)cc3)cc2)cc1] 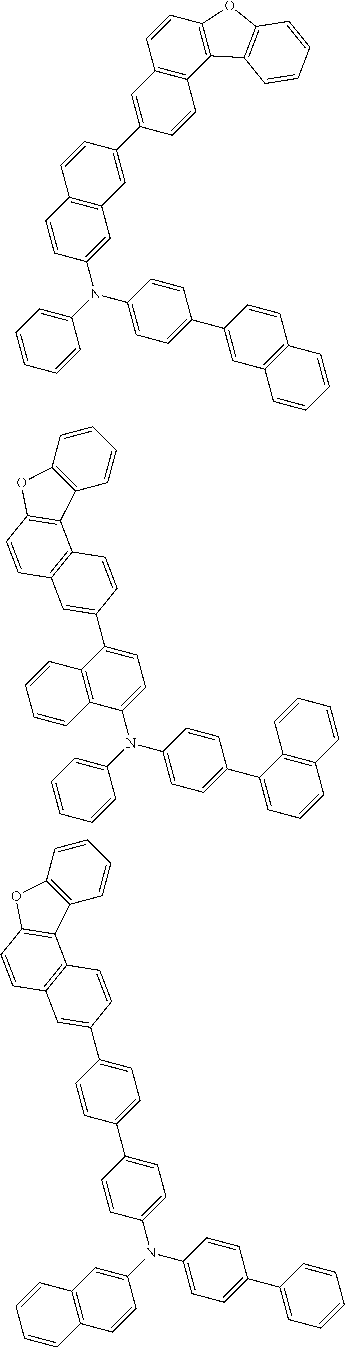 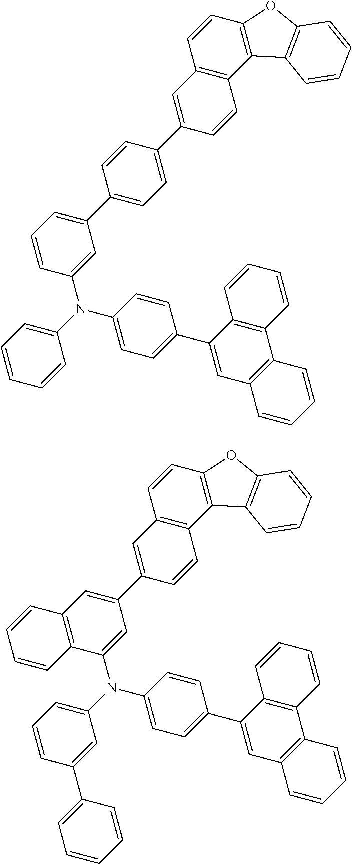 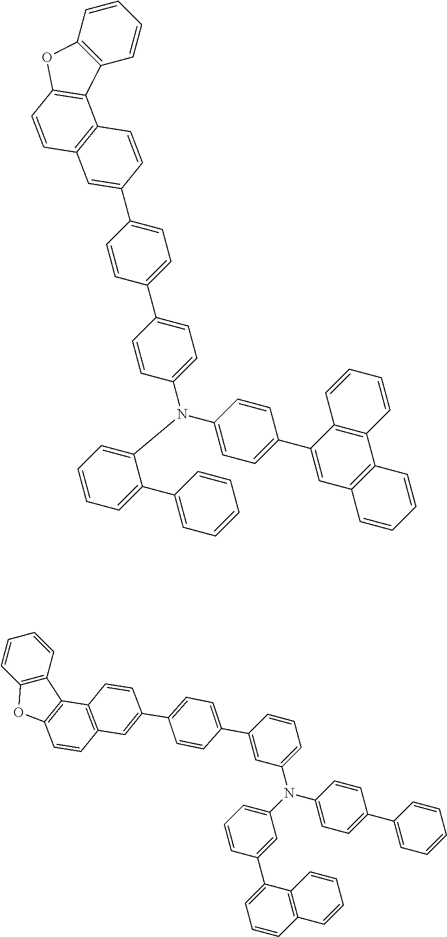 [SMILES: c1ccc(-c2ccccc2N(c2ccc(-c3ccc(-c4ccc5c(ccc6oc7ccccc7c65)c4)cc3)cc2)c2ccc(-c3cc4ccccc4c4ccccc34)cc2)cc1 ; c1ccc(-c2ccc(N(c3cccc(-c4ccc(-c5ccc6c(ccc7oc8ccccc8c76)c5)cc4)c3)c3cccc(-c4cccc5ccccc45)c3)cc2)cc1] 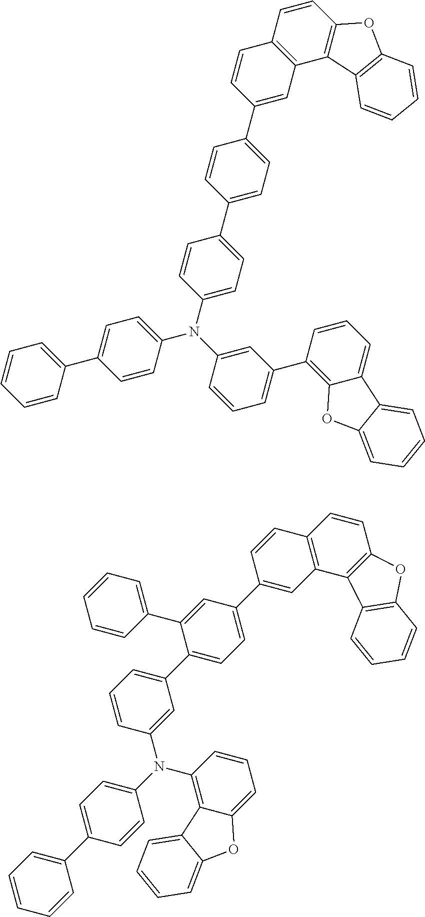 [SMILES: c1ccc(-c2ccc(N(c3ccc(-c4ccc(-c5ccc6ccc7oc8ccccc8c7c6c5)cc4)cc3)c3cccc(-c4cccc5c4oc4ccccc45)c3)cc2)cc1 ; c1ccc(-c2ccc(N(c3cccc(-c4ccc(-c5ccc6ccc7oc8ccccc8c7c6c5)cc4-c4ccccc4)c3)c3cccc4oc5ccccc5c34)cc2)cc1] 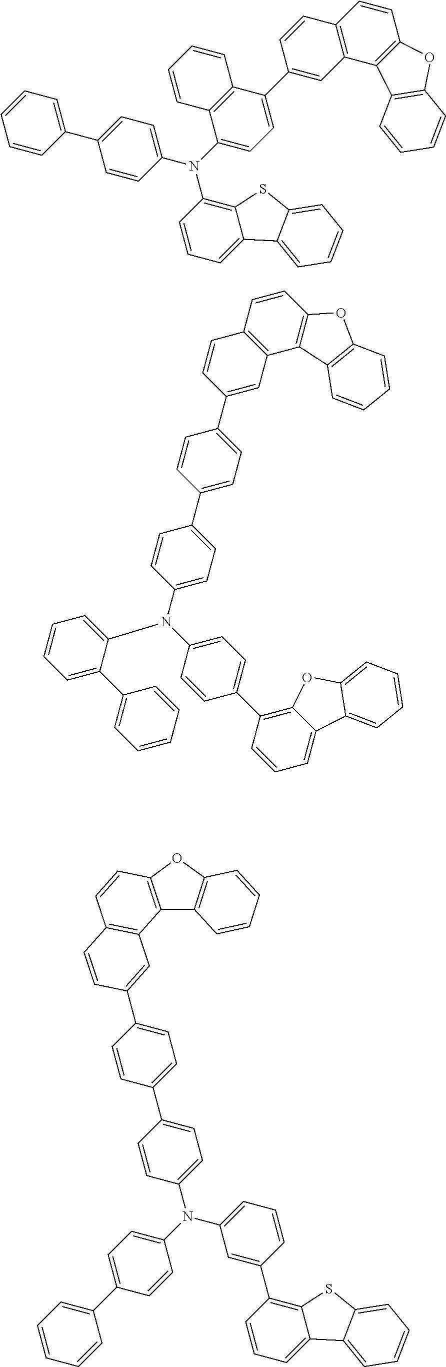 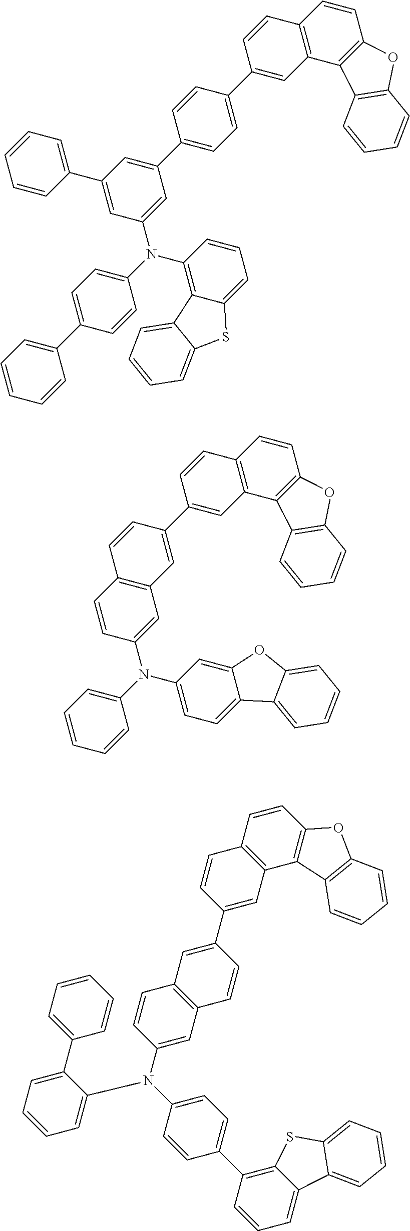 [SMILES: c1ccc(-c2ccc(N(c3cc(-c4ccccc4)cc(-c4ccc(-c5ccc6ccc7oc8ccccc8c7c6c5)cc4)c3)c3cccc4sc5ccccc5c34)cc2)cc1 ; c1ccc(N(c2ccc3ccc(-c4ccc5ccc6oc7ccccc7c6c5c4)cc3c2)c2ccc3c(c2)oc2ccccc23)cc1 ; c1ccc(-c2ccccc2N(c2ccc(-c3cccc4c3sc3ccccc34)cc2)c2ccc3cc(-c4ccc5ccc6oc7ccccc7c6c5c4)ccc3c2)cc1] 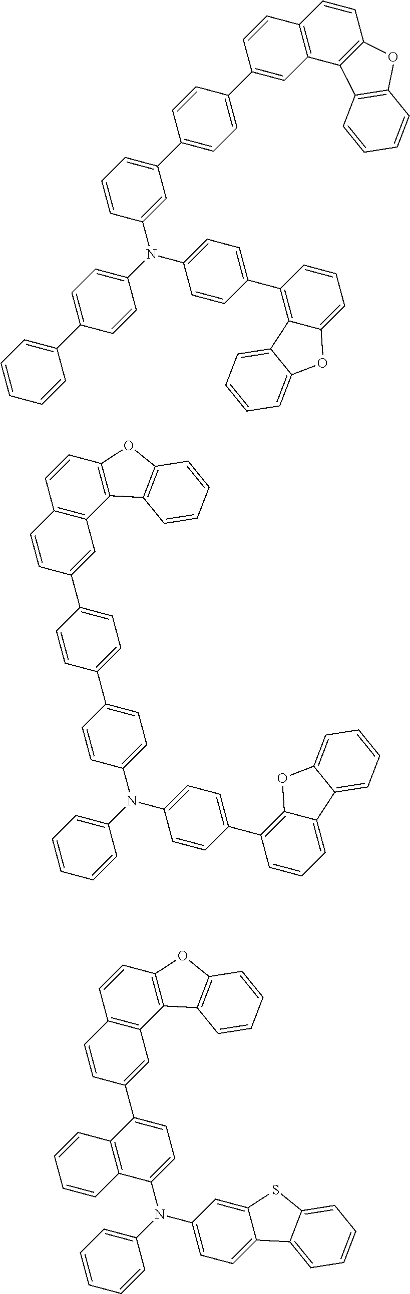 [SMILES: c1ccc(-c2ccc(N(c3ccc(-c4cccc5oc6ccccc6c45)cc3)c3cccc(-c4ccc(-c5ccc6ccc7oc8ccccc8c7c6c5)cc4)c3)cc2)cc1 ; c1ccc(N(c2ccc(-c3ccc(-c4ccc5ccc6oc7ccccc7c6c5c4)cc3)cc2)c2ccc(-c3cccc4c3oc3ccccc34)cc2)cc1 ; c1ccc(N(c2ccc3c(c2)sc2ccccc23)c2ccc(-c3ccc4ccc5oc6ccccc6c5c4c3)c3ccccc23)cc1] 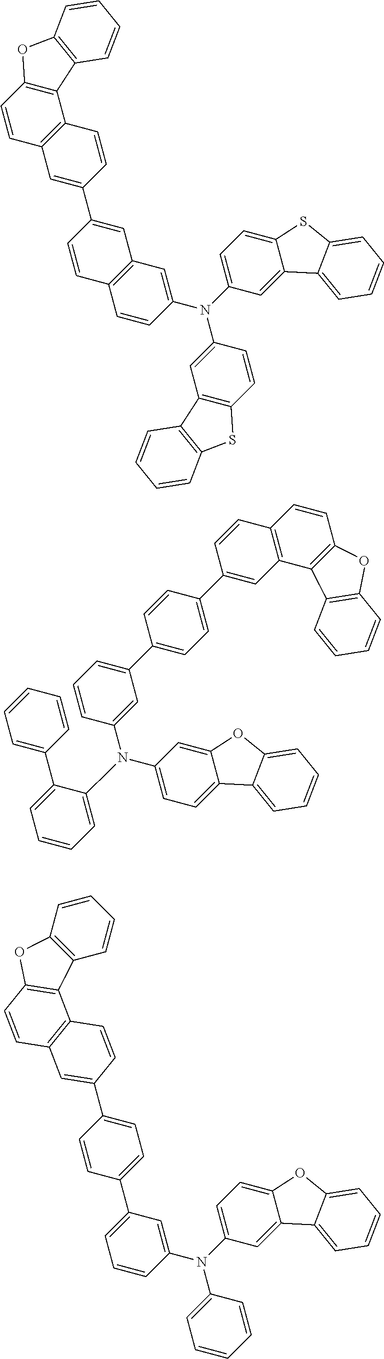 [SMILES: c1ccc2c(c1)oc1ccc3cc(-c4ccc5ccc(N(c6ccc7sc8ccccc8c7c6)c6ccc7sc8ccccc8c7c6)cc5c4)ccc3c12 ; c1ccc(-c2ccccc2N(c2cccc(-c3ccc(-c4ccc5ccc6oc7ccccc7c6c5c4)cc3)c2)c2ccc3c(c2)oc2ccccc23)cc1 ; c1ccc(N(c2cccc(-c3ccc(-c4ccc5c(ccc6oc7ccccc7c65)c4)cc3)c2)c2ccc3oc4ccccc4c3c2)cc1] 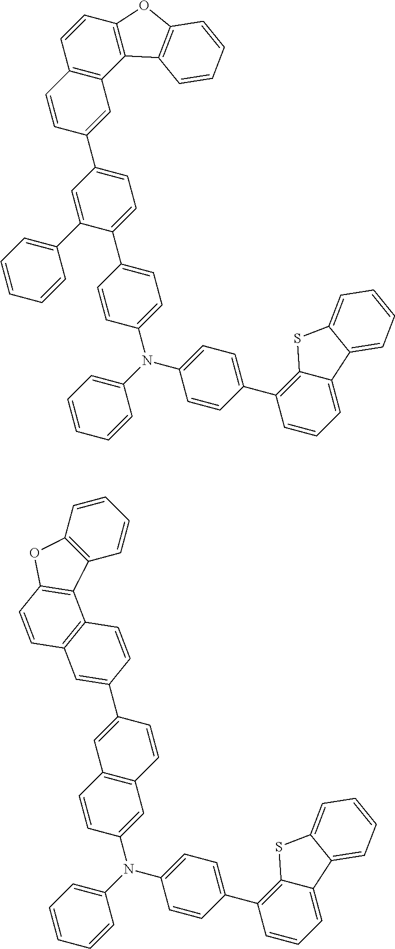 [SMILES: c1ccc(-c2cc(-c3ccc4ccc5oc6ccccc6c5c4c3)ccc2-c2ccc(N(c3ccccc3)c3ccc(-c4cccc5c4sc4ccccc45)cc3)cc2)cc1 ; c1ccc(N(c2ccc(-c3cccc4c3sc3ccccc34)cc2)c2ccc3cc(-c4ccc5c(ccc6oc7ccccc7c65)c4)ccc3c2)cc1] 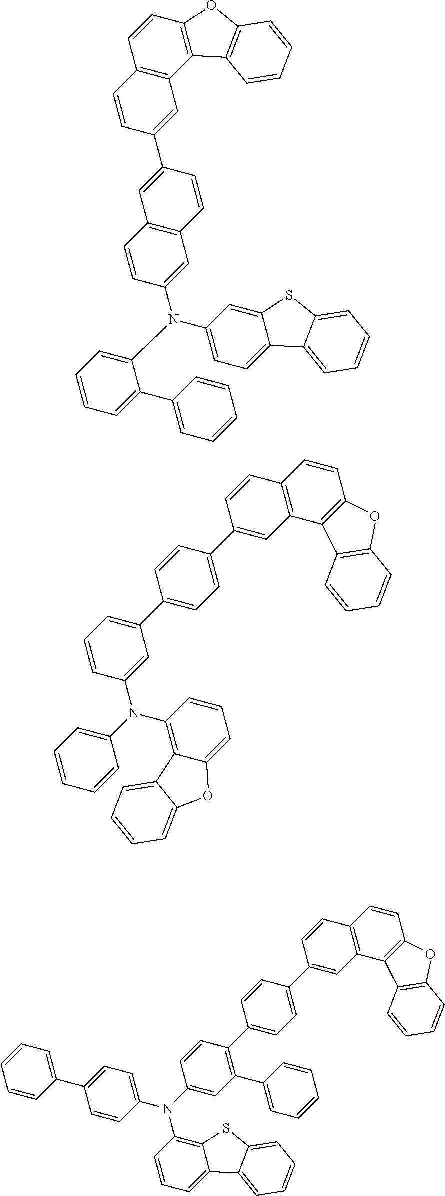 [SMILES: c1ccc(-c2ccccc2N(c2ccc3cc(-c4ccc5ccc6oc7ccccc7c6c5c4)ccc3c2)c2ccc3c(c2)sc2ccccc23)cc1 ; c1ccc(N(c2cccc(-c3ccc(-c4ccc5ccc6oc7ccccc7c6c5c4)cc3)c2)c2cccc3oc4ccccc4c23)cc1 ; c1ccc(-c2ccc(N(c3ccc(-c4ccc(-c5ccc6ccc7oc8ccccc8c7c6c5)cc4)c(-c4ccccc4)c3)c3cccc4c3sc3ccccc34)cc2)cc1] 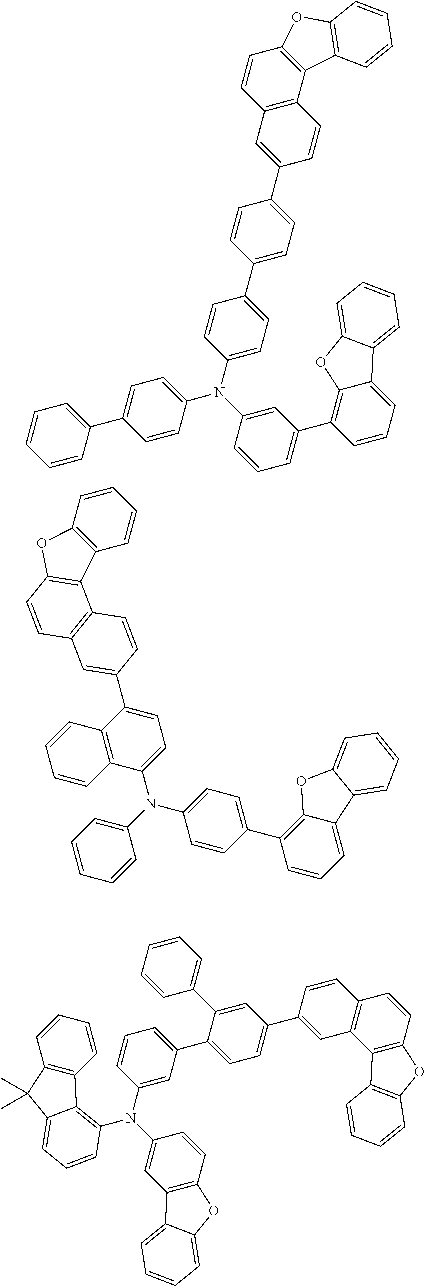 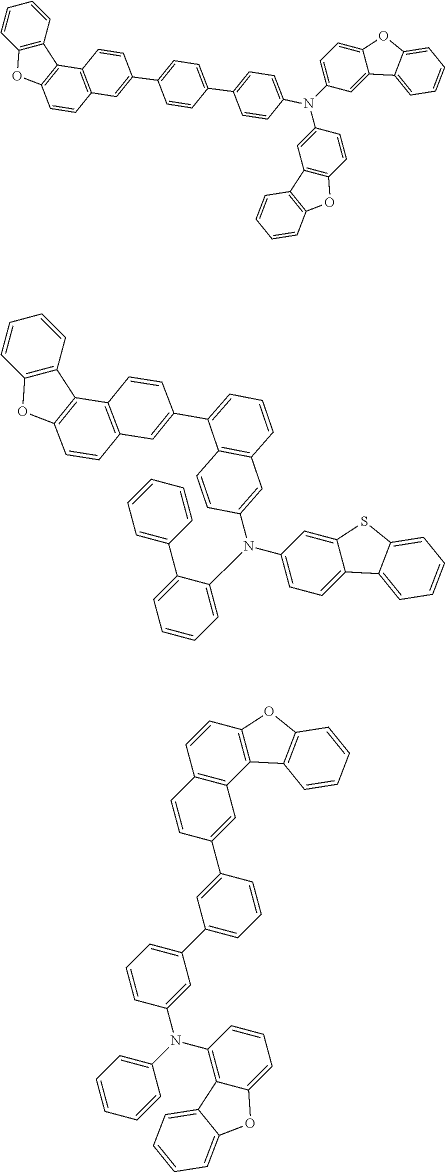 [SMILES: c1ccc2c(c1)oc1ccc(N(c3ccc(-c4ccc(-c5ccc6c(ccc7oc8ccccc8c76)c5)cc4)cc3)c3ccc4oc5ccccc5c4c3)cc12 ; c1ccc(-c2ccccc2N(c2ccc3c(-c4ccc5c(ccc6oc7ccccc7c65)c4)cccc3c2)c2ccc3c(c2)sc2ccccc23)cc1 ; c1ccc(N(c2cccc(-c3cccc(-c4ccc5ccc6oc7ccccc7c6c5c4)c3)c2)c2cccc3oc4ccccc4c23)cc1] 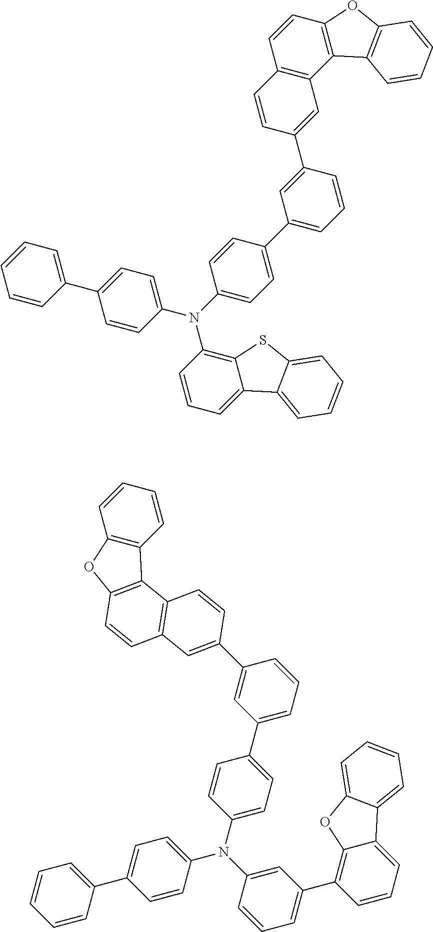 [SMILES: c1ccc(-c2ccc(N(c3ccc(-c4cccc(-c5ccc6ccc7oc8ccccc8c7c6c5)c4)cc3)c3cccc4c3sc3ccccc34)cc2)cc1 ; c1ccc(-c2ccc(N(c3ccc(-c4cccc(-c5ccc6c(ccc7oc8ccccc8c76)c5)c4)cc3)c3cccc(-c4cccc5c4oc4ccccc45)c3)cc2)cc1] 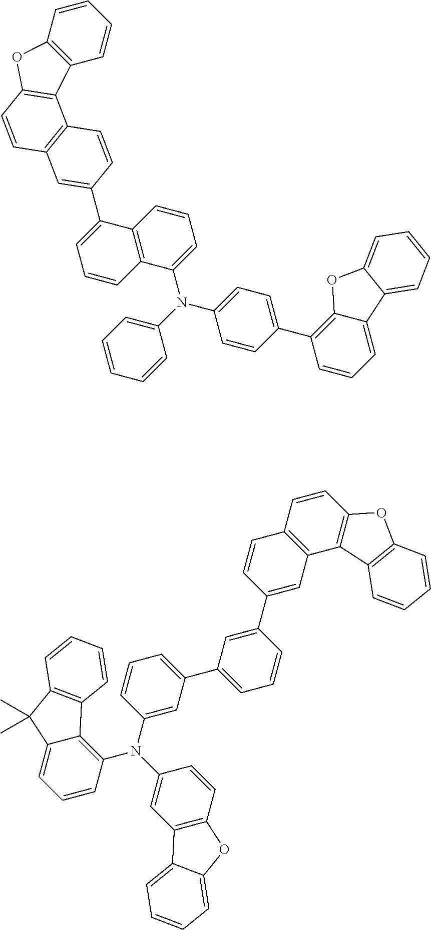 [SMILES: c1ccc(N(c2ccc(-c3cccc4c3oc3ccccc34)cc2)c2cccc3c(-c4ccc5c(ccc6oc7ccccc7c65)c4)cccc23)cc1 ; CC1(C)c2ccccc2-c2c(N(c3cccc(-c4cccc(-c5ccc6ccc7oc8ccccc8c7c6c5)c4)c3)c3ccc4oc5ccccc5c4c3)cccc21] 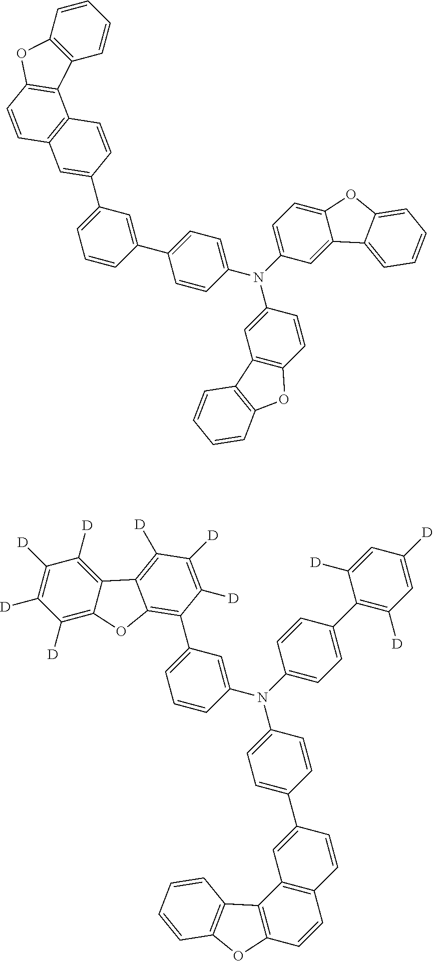 [SMILES: c1cc(-c2ccc(N(c3ccc4oc5ccccc5c4c3)c3ccc4oc5ccccc5c4c3)cc2)cc(-c2ccc3c(ccc4oc5ccccc5c43)c2)c1 ; [2H]c1cc([2H])c(-c2ccc(N(c3ccc(-c4ccc5ccc6oc7ccccc7c6c5c4)cc3)c3cccc(-c4c([2H])c([2H])c([2H])c5c4oc4c([2H])c([2H])c([2H])c([2H])c45)c3)cc2)c([2H])c1] 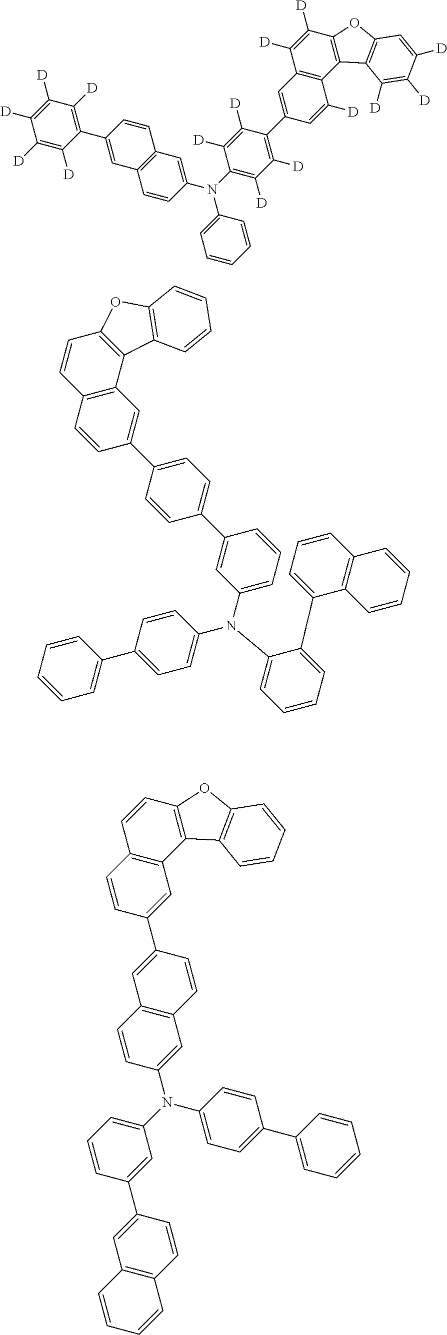 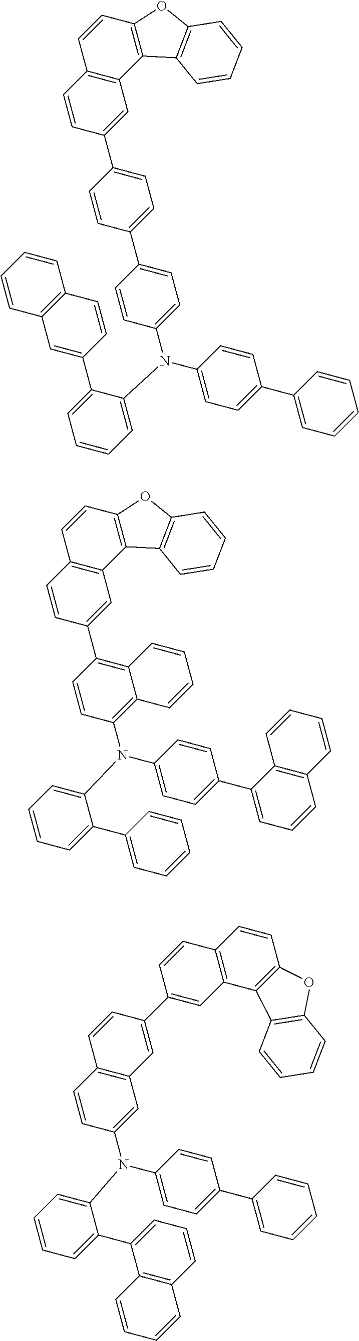 [SMILES: c1ccc(-c2ccc(N(c3ccc(-c4ccc(-c5ccc6ccc7oc8ccccc8c7c6c5)cc4)cc3)c3ccccc3-c3ccc4ccccc4c3)cc2)cc1 ; c1ccc(-c2ccccc2N(c2ccc(-c3cccc4ccccc34)cc2)c2ccc(-c3ccc4ccc5oc6ccccc6c5c4c3)c3ccccc23)cc1 ; c1ccc(-c2ccc(N(c3ccc4ccc(-c5ccc6ccc7oc8ccccc8c7c6c5)cc4c3)c3ccccc3-c3cccc4ccccc34)cc2)cc1] 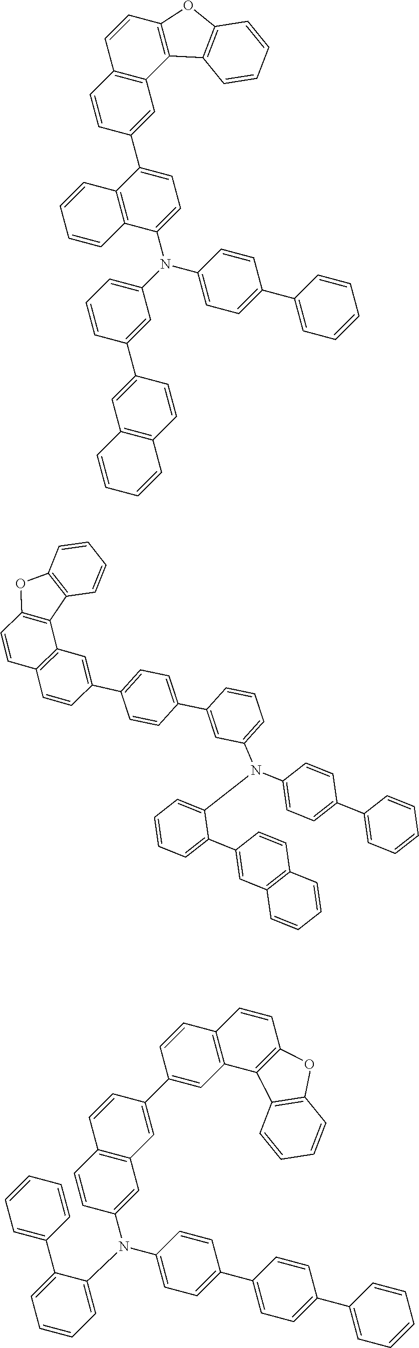 [SMILES: c1ccc(-c2ccc(N(c3cccc(-c4ccc5ccccc5c4)c3)c3ccc(-c4ccc5ccc6oc7ccccc7c6c5c4)c4ccccc34)cc2)cc1 ; c1ccc(-c2ccc(N(c3cccc(-c4ccc(-c5ccc6ccc7oc8ccccc8c7c6c5)cc4)c3)c3ccccc3-c3ccc4ccccc4c3)cc2)cc1 ; c1ccc(-c2ccc(-c3ccc(N(c4ccc5ccc(-c6ccc7ccc8oc9ccccc9c8c7c6)cc5c4)c4ccccc4-c4ccccc4)cc3)cc2)cc1] 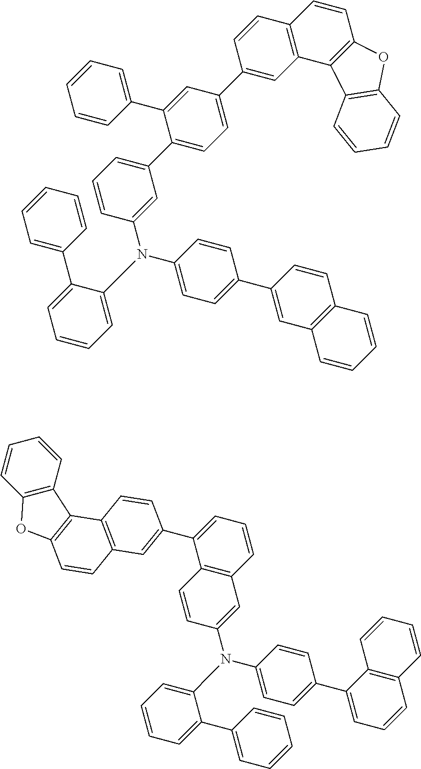 [SMILES: c1ccc(-c2cc(-c3ccc4ccc5oc6ccccc6c5c4c3)ccc2-c2cccc(N(c3ccc(-c4ccc5ccccc5c4)cc3)c3ccccc3-c3ccccc3)c2)cc1 ; c1ccc(-c2ccccc2N(c2ccc(-c3cccc4ccccc34)cc2)c2ccc3c(-c4ccc5c(ccc6oc7ccccc7c65)c4)cccc3c2)cc1] 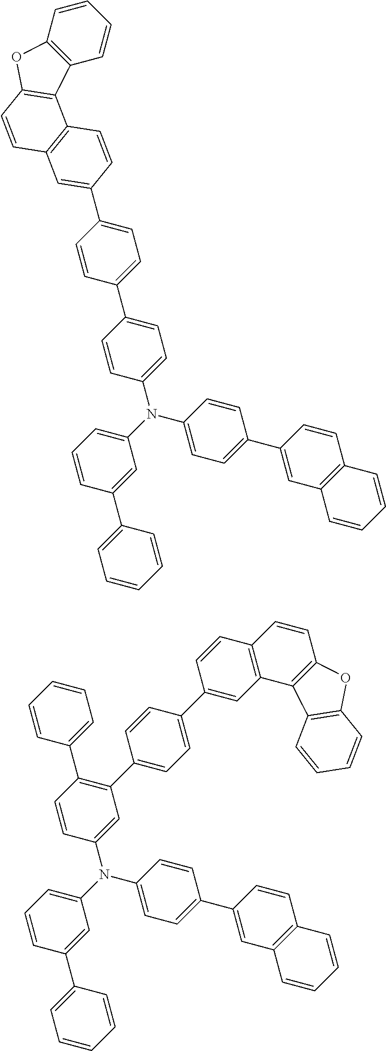 [SMILES: c1ccc(-c2cccc(N(c3ccc(-c4ccc(-c5ccc6c(ccc7oc8ccccc8c76)c5)cc4)cc3)c3ccc(-c4ccc5ccccc5c4)cc3)c2)cc1 ; c1ccc(-c2cccc(N(c3ccc(-c4ccc5ccccc5c4)cc3)c3ccc(-c4ccccc4)c(-c4ccc(-c5ccc6ccc7oc8ccccc8c7c6c5)cc4)c3)c2)cc1] 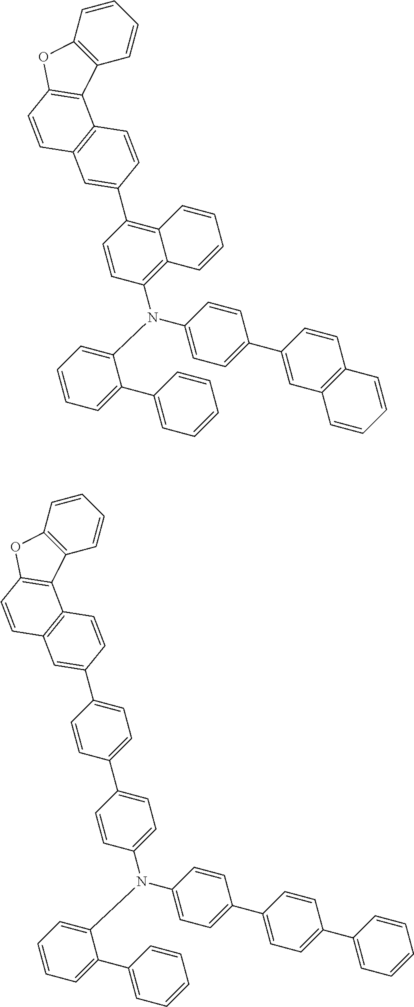 [SMILES: c1ccc(-c2ccccc2N(c2ccc(-c3ccc4ccccc4c3)cc2)c2ccc(-c3ccc4c(ccc5oc6ccccc6c54)c3)c3ccccc23)cc1 ; c1ccc(-c2ccc(-c3ccc(N(c4ccc(-c5ccc(-c6ccc7c(ccc8oc9ccccc9c87)c6)cc5)cc4)c4ccccc4-c4ccccc4)cc3)cc2)cc1] 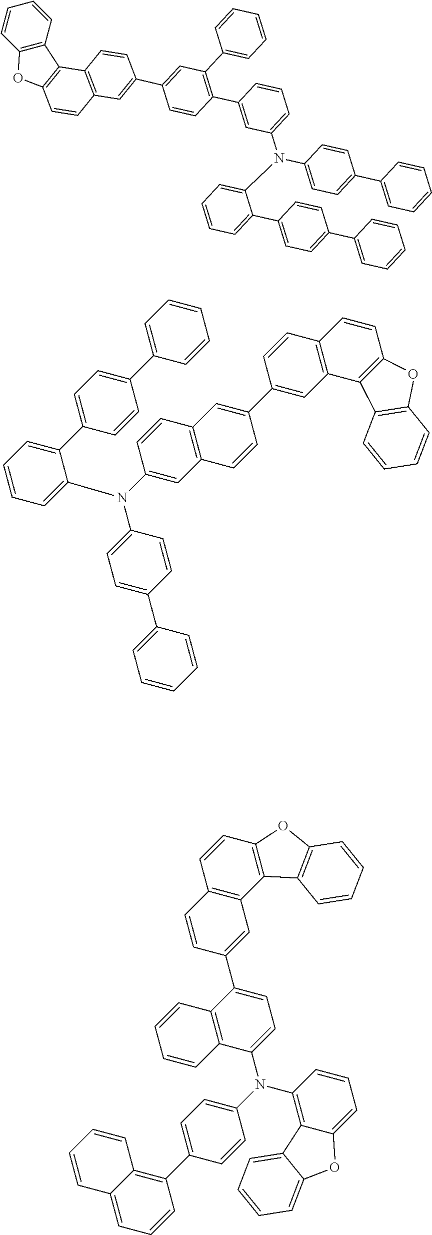 [SMILES: c1ccc(-c2ccc(-c3ccccc3N(c3ccc(-c4ccccc4)cc3)c3cccc(-c4ccc(-c5ccc6c(ccc7oc8ccccc8c76)c5)cc4-c4ccccc4)c3)cc2)cc1 ; c1ccc(-c2ccc(-c3ccccc3N(c3ccc(-c4ccccc4)cc3)c3ccc4cc(-c5ccc6ccc7oc8ccccc8c7c6c5)ccc4c3)cc2)cc1 ; c1ccc2c(-c3ccc(N(c4ccc(-c5ccc6ccc7oc8ccccc8c7c6c5)c5ccccc45)c4cccc5oc6ccccc6c45)cc3)cccc2c1] 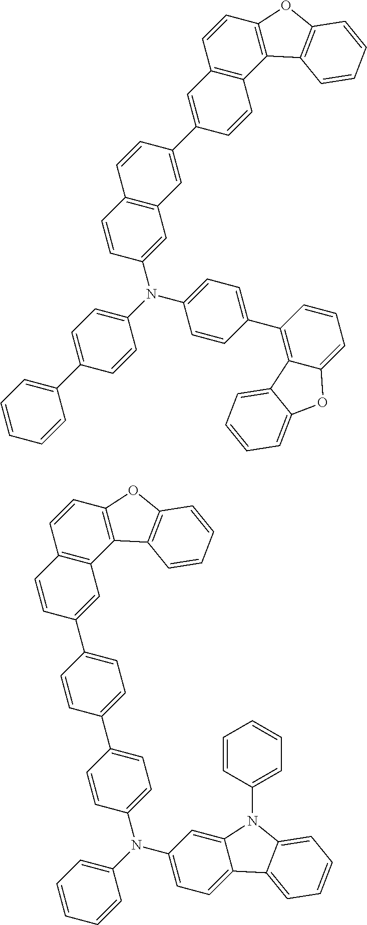 [SMILES: c1ccc(-c2ccc(N(c3ccc(-c4cccc5oc6ccccc6c45)cc3)c3ccc4ccc(-c5ccc6c(ccc7oc8ccccc8c76)c5)cc4c3)cc2)cc1 ; c1ccc(N(c2ccc(-c3ccc(-c4ccc5ccc6oc7ccccc7c6c5c4)cc3)cc2)c2ccc3c4ccccc4n(-c4ccccc4)c3c2)cc1] 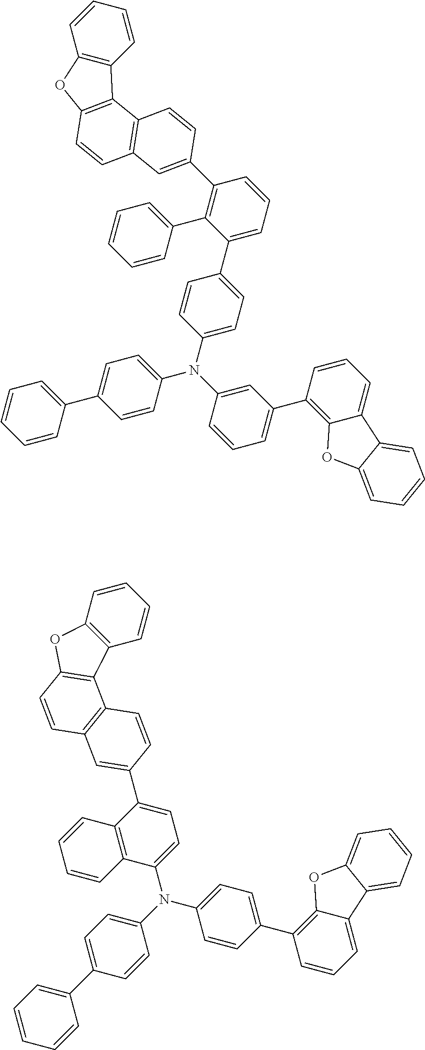 [SMILES: c1ccc(-c2ccc(N(c3ccc(-c4cccc(-c5ccc6c(ccc7oc8ccccc8c76)c5)c4-c4ccccc4)cc3)c3cccc(-c4cccc5c4oc4ccccc45)c3)cc2)cc1 ; c1ccc(-c2ccc(N(c3ccc(-c4cccc5c4oc4ccccc45)cc3)c3ccc(-c4ccc5c(ccc6oc7ccccc7c65)c4)c4ccccc34)cc2)cc1] 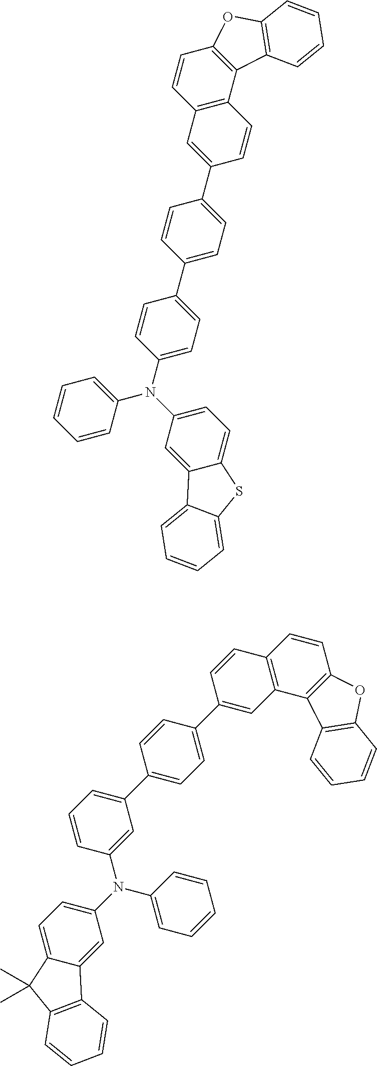 [SMILES: c1ccc(N(c2ccc(-c3ccc(-c4ccc5c(ccc6oc7ccccc7c65)c4)cc3)cc2)c2ccc3sc4ccccc4c3c2)cc1 ; CC1(C)c2ccccc2-c2cc(N(c3ccccc3)c3cccc(-c4ccc(-c5ccc6ccc7oc8ccccc8c7c6c5)cc4)c3)ccc21] 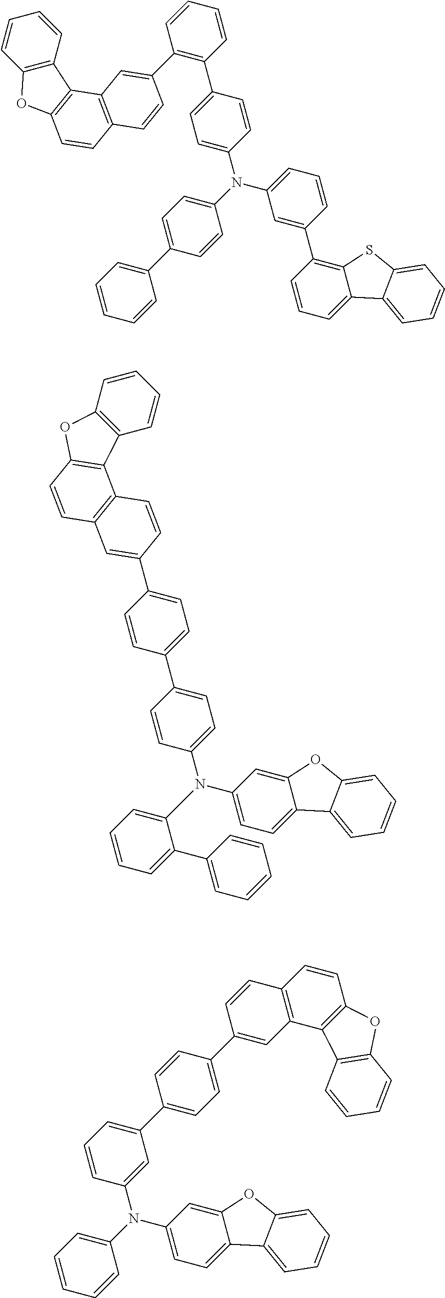 [SMILES: c1ccc(-c2ccc(N(c3ccc(-c4ccccc4-c4ccc5ccc6oc7ccccc7c6c5c4)cc3)c3cccc(-c4cccc5c4sc4ccccc45)c3)cc2)cc1 ; c1ccc(-c2ccccc2N(c2ccc(-c3ccc(-c4ccc5c(ccc6oc7ccccc7c65)c4)cc3)cc2)c2ccc3c(c2)oc2ccccc23)cc1 ; c1ccc(N(c2cccc(-c3ccc(-c4ccc5ccc6oc7ccccc7c6c5c4)cc3)c2)c2ccc3c(c2)oc2ccccc23)cc1] 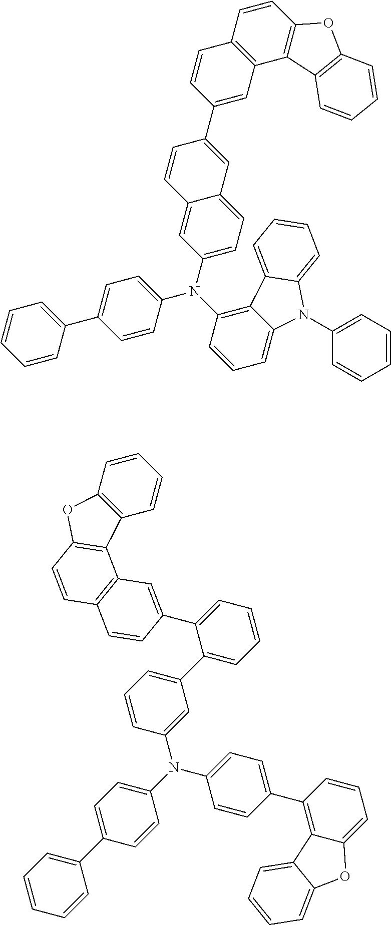 [SMILES: c1ccc(-c2ccc(N(c3ccc4cc(-c5ccc6ccc7oc8ccccc8c7c6c5)ccc4c3)c3cccc4c3c3ccccc3n4-c3ccccc3)cc2)cc1 ; c1ccc(-c2ccc(N(c3ccc(-c4cccc5oc6ccccc6c45)cc3)c3cccc(-c4ccccc4-c4ccc5ccc6oc7ccccc7c6c5c4)c3)cc2)cc1] 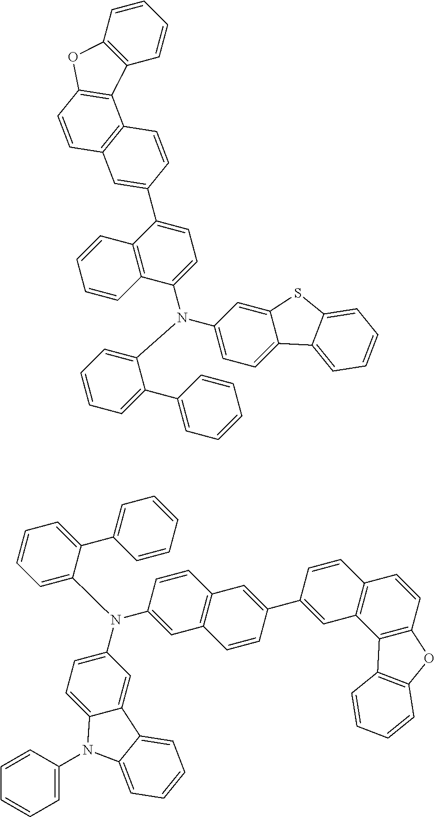 [SMILES: c1ccc(-c2ccccc2N(c2ccc3c(c2)sc2ccccc23)c2ccc(-c3ccc4c(ccc5oc6ccccc6c54)c3)c3ccccc23)cc1 ; c1ccc(-c2ccccc2N(c2ccc3cc(-c4ccc5ccc6oc7ccccc7c6c5c4)ccc3c2)c2ccc3c(c2)c2ccccc2n3-c2ccccc2)cc1] 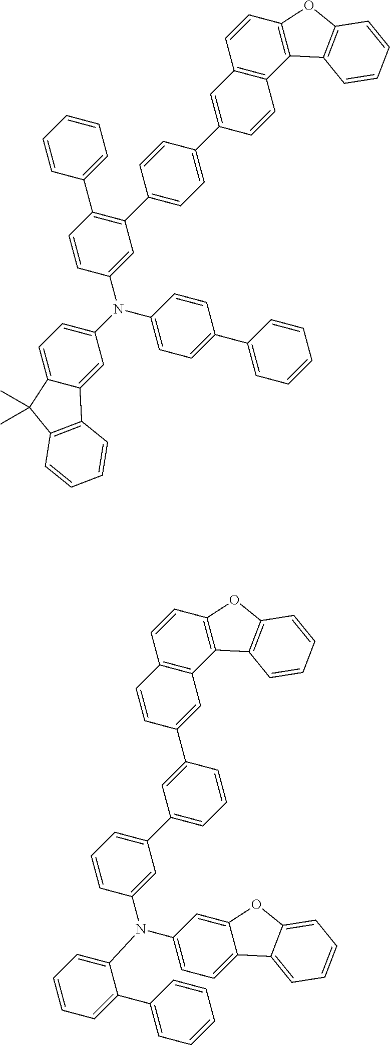 [SMILES: CC1(C)c2ccccc2-c2cc(N(c3ccc(-c4ccccc4)cc3)c3ccc(-c4ccccc4)c(-c4ccc(-c5ccc6c(ccc7oc8ccccc8c76)c5)cc4)c3)ccc21 ; c1ccc(-c2ccccc2N(c2cccc(-c3cccc(-c4ccc5ccc6oc7ccccc7c6c5c4)c3)c2)c2ccc3c(c2)oc2ccccc23)cc1] 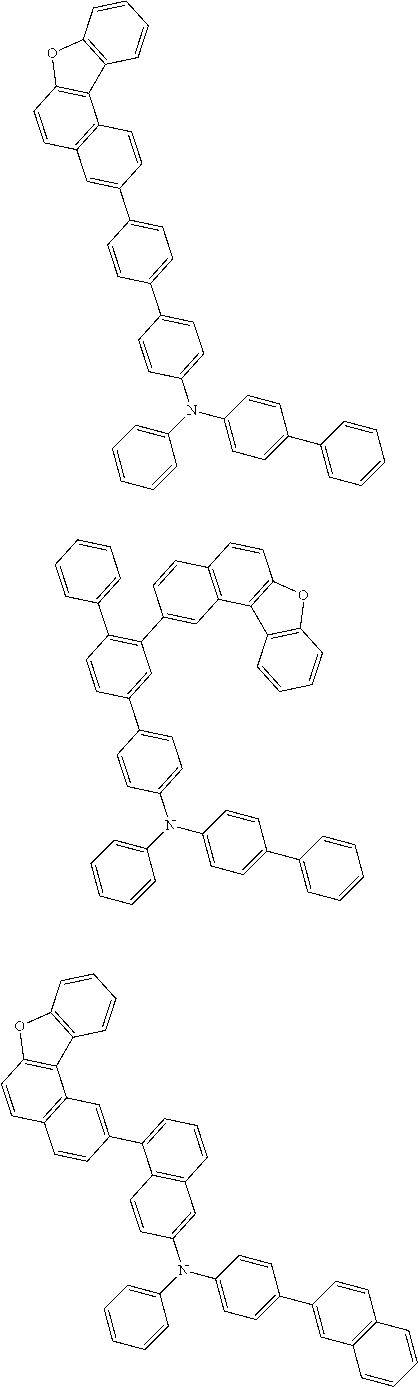 [SMILES: c1ccc(-c2ccc(N(c3ccccc3)c3ccc(-c4ccc(-c5ccc6c(ccc7oc8ccccc8c76)c5)cc4)cc3)cc2)cc1 ; c1ccc(-c2ccc(N(c3ccccc3)c3ccc(-c4ccc(-c5ccccc5)c(-c5ccc6ccc7oc8ccccc8c7c6c5)c4)cc3)cc2)cc1 ; c1ccc(N(c2ccc(-c3ccc4ccccc4c3)cc2)c2ccc3c(-c4ccc5ccc6oc7ccccc7c6c5c4)cccc3c2)cc1] 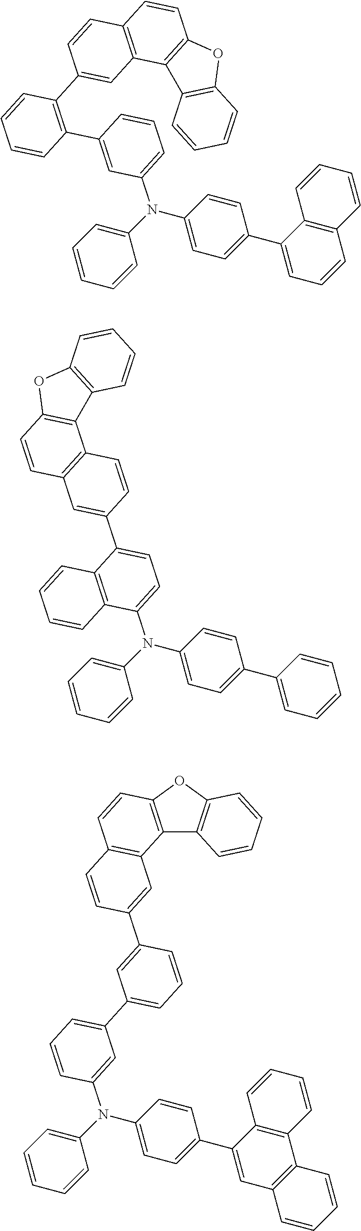 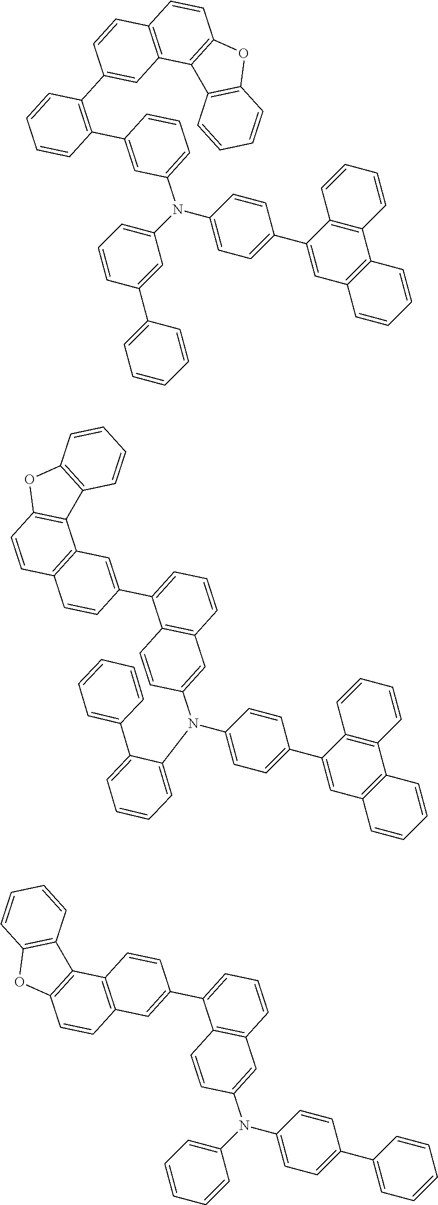 [SMILES: c1ccc(-c2cccc(N(c3ccc(-c4cc5ccccc5c5ccccc45)cc3)c3cccc(-c4ccccc4-c4ccc5ccc6oc7ccccc7c6c5c4)c3)c2)cc1 ; c1ccc(-c2ccccc2N(c2ccc(-c3cc4ccccc4c4ccccc34)cc2)c2ccc3c(-c4ccc5ccc6oc7ccccc7c6c5c4)cccc3c2)cc1 ; c1ccc(-c2ccc(N(c3ccccc3)c3ccc4c(-c5ccc6c(ccc7oc8ccccc8c76)c5)cccc4c3)cc2)cc1] 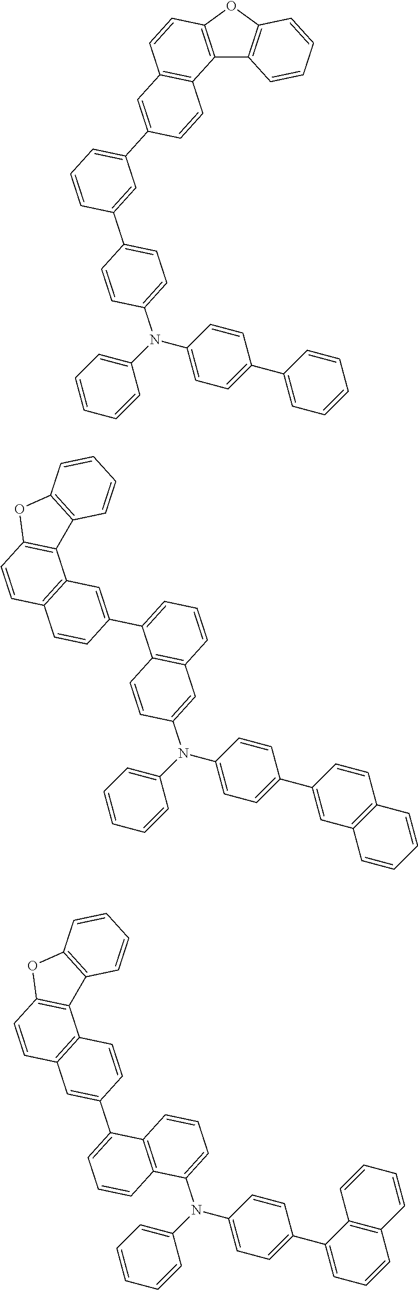 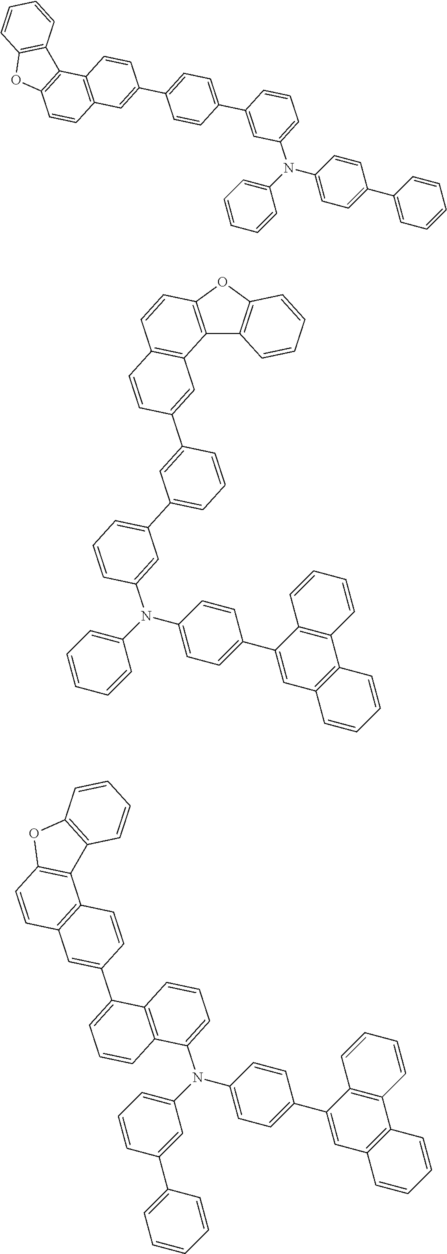 [SMILES: c1ccc(-c2ccc(N(c3ccccc3)c3cccc(-c4ccc(-c5ccc6c(ccc7oc8ccccc8c76)c5)cc4)c3)cc2)cc1 ; c1ccc(N(c2ccc(-c3cc4ccccc4c4ccccc34)cc2)c2cccc(-c3cccc(-c4ccc5ccc6oc7ccccc7c6c5c4)c3)c2)cc1 ; c1ccc(-c2cccc(N(c3ccc(-c4cc5ccccc5c5ccccc45)cc3)c3cccc4c(-c5ccc6c(ccc7oc8ccccc8c76)c5)cccc34)c2)cc1] 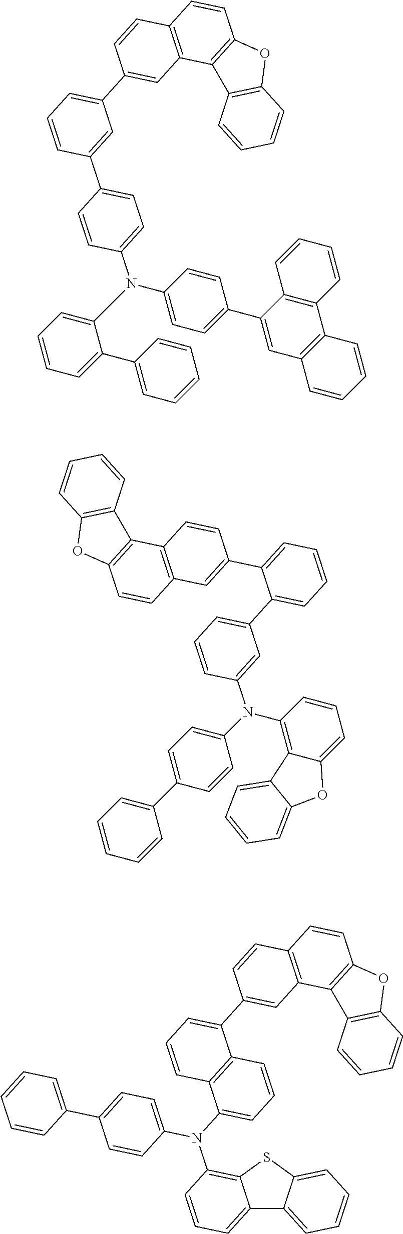 [SMILES: c1ccc(-c2ccccc2N(c2ccc(-c3cccc(-c4ccc5ccc6oc7ccccc7c6c5c4)c3)cc2)c2ccc(-c3cc4ccccc4c4ccccc34)cc2)cc1 ; c1ccc(-c2ccc(N(c3cccc(-c4ccccc4-c4ccc5c(ccc6oc7ccccc7c65)c4)c3)c3cccc4oc5ccccc5c34)cc2)cc1 ; c1ccc(-c2ccc(N(c3cccc4c(-c5ccc6ccc7oc8ccccc8c7c6c5)cccc34)c3cccc4c3sc3ccccc34)cc2)cc1] 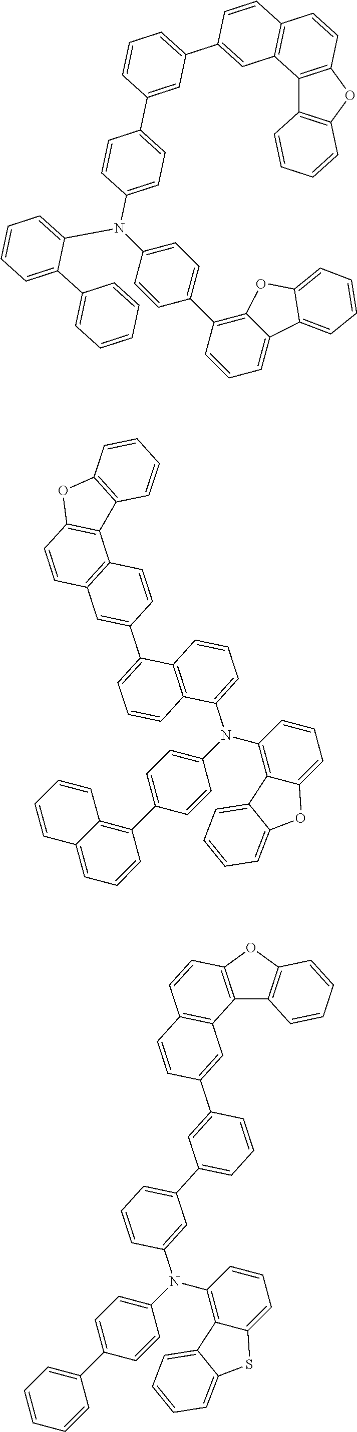 [SMILES: c1ccc(-c2ccccc2N(c2ccc(-c3cccc(-c4ccc5ccc6oc7ccccc7c6c5c4)c3)cc2)c2ccc(-c3cccc4c3oc3ccccc34)cc2)cc1 ; c1ccc2c(-c3ccc(N(c4cccc5c(-c6ccc7c(ccc8oc9ccccc9c87)c6)cccc45)c4cccc5oc6ccccc6c45)cc3)cccc2c1 ; c1ccc(-c2ccc(N(c3cccc(-c4cccc(-c5ccc6ccc7oc8ccccc8c7c6c5)c4)c3)c3cccc4sc5ccccc5c34)cc2)cc1] 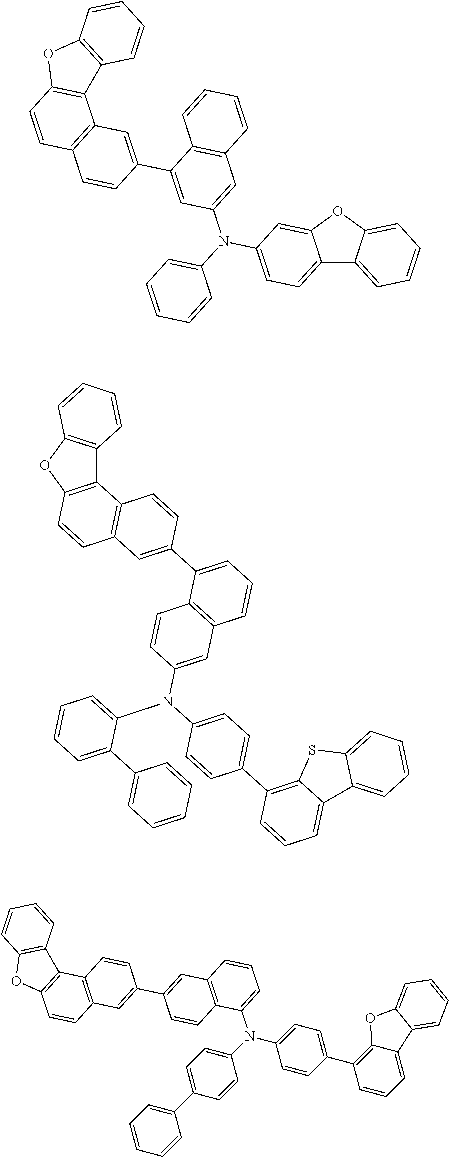 [SMILES: c1ccc(N(c2cc(-c3ccc4ccc5oc6ccccc6c5c4c3)c3ccccc3c2)c2ccc3c(c2)oc2ccccc23)cc1 ; c1ccc(-c2ccccc2N(c2ccc(-c3cccc4c3sc3ccccc34)cc2)c2ccc3c(-c4ccc5c(ccc6oc7ccccc7c65)c4)cccc3c2)cc1 ; c1ccc(-c2ccc(N(c3ccc(-c4cccc5c4oc4ccccc45)cc3)c3cccc4cc(-c5ccc6c(ccc7oc8ccccc8c76)c5)ccc34)cc2)cc1] 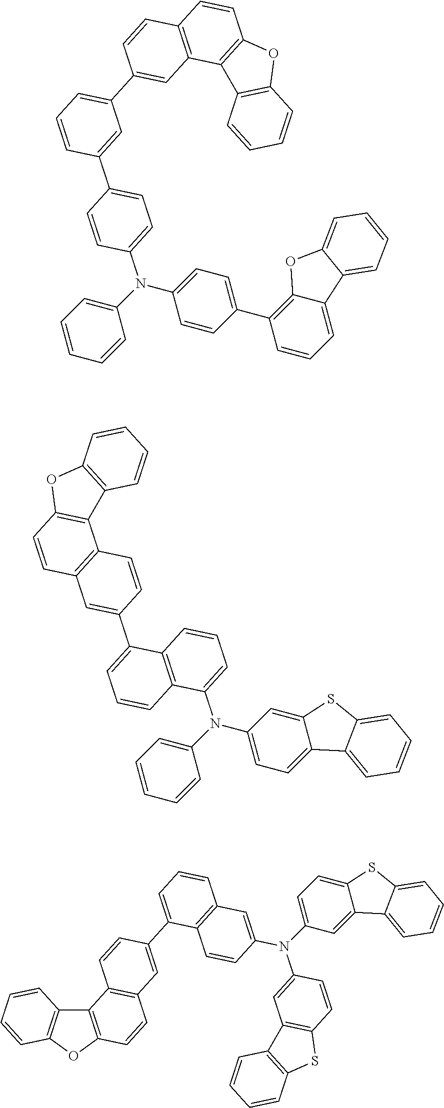 [SMILES: c1ccc(N(c2ccc(-c3cccc(-c4ccc5ccc6oc7ccccc7c6c5c4)c3)cc2)c2ccc(-c3cccc4c3oc3ccccc34)cc2)cc1 ; c1ccc(N(c2ccc3c(c2)sc2ccccc23)c2cccc3c(-c4ccc5c(ccc6oc7ccccc7c65)c4)cccc23)cc1 ; c1cc(-c2ccc3c(ccc4oc5ccccc5c43)c2)c2ccc(N(c3ccc4sc5ccccc5c4c3)c3ccc4sc5ccccc5c4c3)cc2c1] 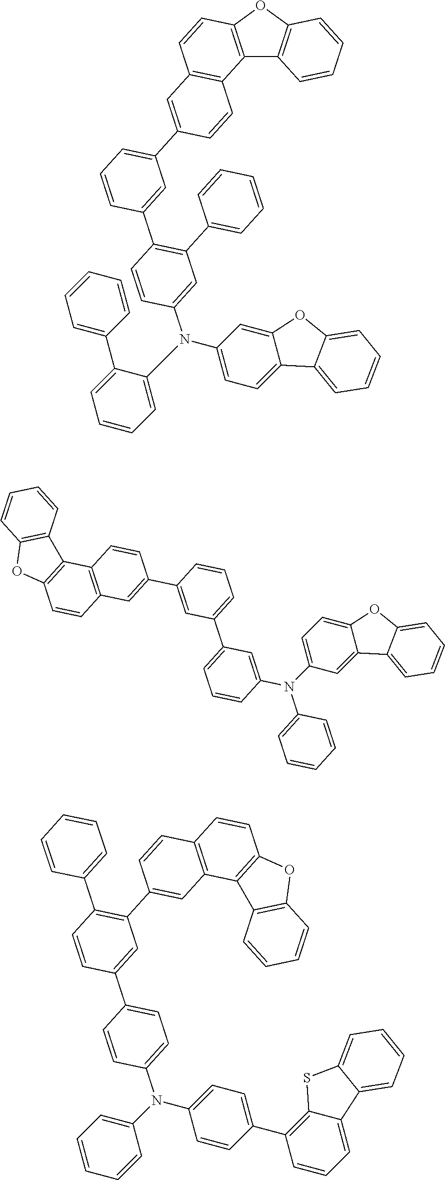 [SMILES: c1ccc(-c2cc(N(c3ccc4c(c3)oc3ccccc34)c3ccccc3-c3ccccc3)ccc2-c2cccc(-c3ccc4c(ccc5oc6ccccc6c54)c3)c2)cc1 ; c1ccc(N(c2cccc(-c3cccc(-c4ccc5c(ccc6oc7ccccc7c65)c4)c3)c2)c2ccc3oc4ccccc4c3c2)cc1 ; c1ccc(-c2ccc(-c3ccc(N(c4ccccc4)c4ccc(-c5cccc6c5sc5ccccc56)cc4)cc3)cc2-c2ccc3ccc4oc5ccccc5c4c3c2)cc1] 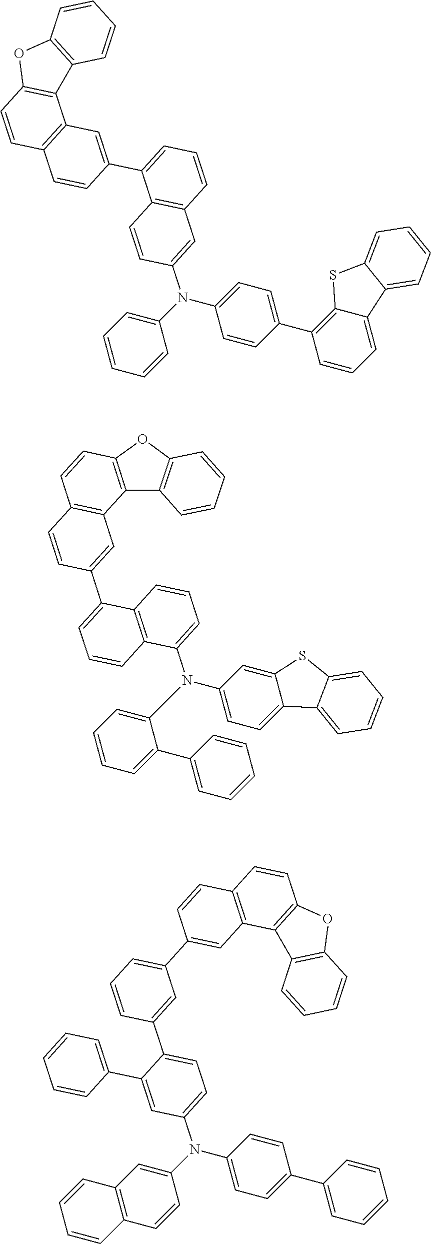 [SMILES: c1ccc(N(c2ccc(-c3cccc4c3sc3ccccc34)cc2)c2ccc3c(-c4ccc5ccc6oc7ccccc7c6c5c4)cccc3c2)cc1 ; c1ccc(-c2ccccc2N(c2ccc3c(c2)sc2ccccc23)c2cccc3c(-c4ccc5ccc6oc7ccccc7c6c5c4)cccc23)cc1 ; c1ccc(-c2ccc(N(c3ccc(-c4cccc(-c5ccc6ccc7oc8ccccc8c7c6c5)c4)c(-c4ccccc4)c3)c3ccc4ccccc4c3)cc2)cc1] 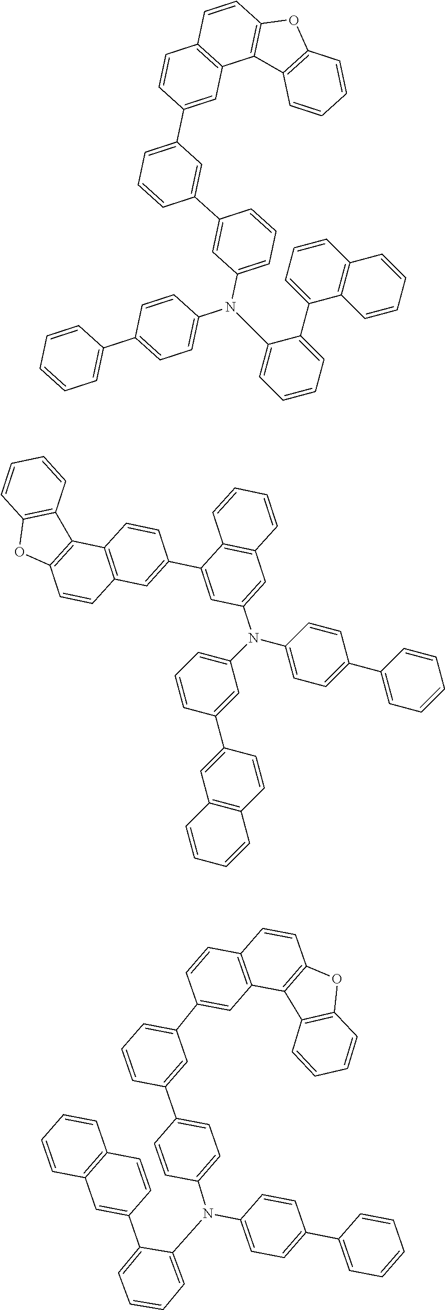 [SMILES: c1ccc(-c2ccc(N(c3cccc(-c4cccc(-c5ccc6ccc7oc8ccccc8c7c6c5)c4)c3)c3ccccc3-c3cccc4ccccc34)cc2)cc1 ; c1ccc(-c2ccc(N(c3cccc(-c4ccc5ccccc5c4)c3)c3cc(-c4ccc5c(ccc6oc7ccccc7c65)c4)c4ccccc4c3)cc2)cc1 ; c1ccc(-c2ccc(N(c3ccc(-c4cccc(-c5ccc6ccc7oc8ccccc8c7c6c5)c4)cc3)c3ccccc3-c3ccc4ccccc4c3)cc2)cc1] 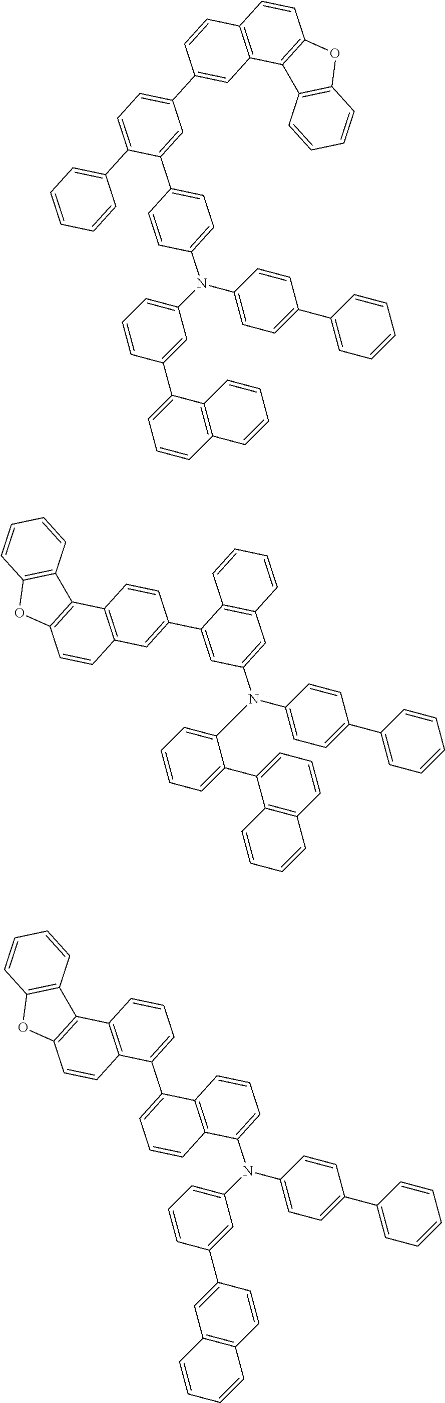 [SMILES: c1ccc(-c2ccc(N(c3ccc(-c4cc(-c5ccc6ccc7oc8ccccc8c7c6c5)ccc4-c4ccccc4)cc3)c3cccc(-c4cccc5ccccc45)c3)cc2)cc1 ; c1ccc(-c2ccc(N(c3cc(-c4ccc5c(ccc6oc7ccccc7c65)c4)c4ccccc4c3)c3ccccc3-c3cccc4ccccc34)cc2)cc1 ; c1ccc(-c2ccc(N(c3cccc(-c4ccc5ccccc5c4)c3)c3cccc4c(-c5cccc6c5ccc5oc7ccccc7c56)cccc34)cc2)cc1] 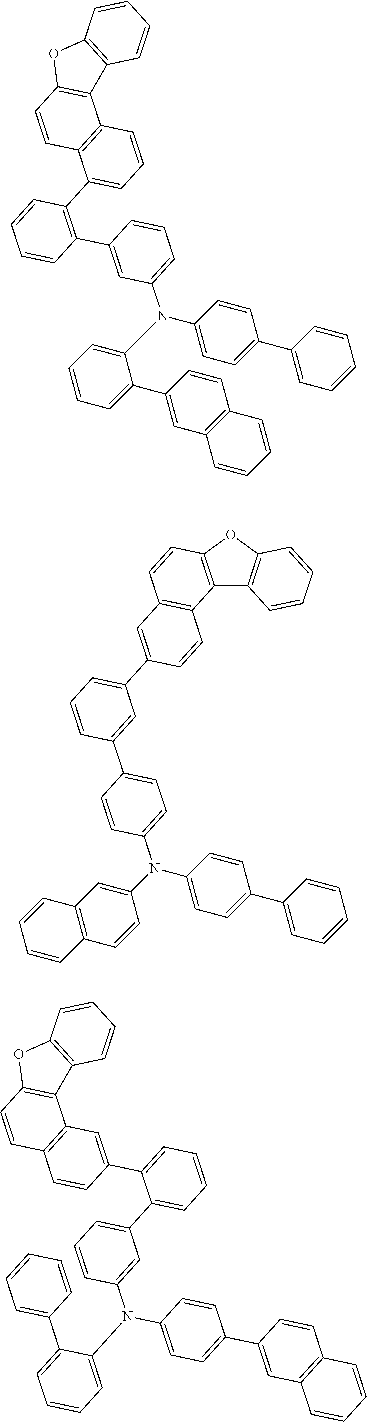 [SMILES: c1ccc(-c2ccc(N(c3cccc(-c4ccccc4-c4cccc5c4ccc4oc6ccccc6c45)c3)c3ccccc3-c3ccc4ccccc4c3)cc2)cc1 ; c1ccc(-c2ccc(N(c3ccc(-c4cccc(-c5ccc6c(ccc7oc8ccccc8c76)c5)c4)cc3)c3ccc4ccccc4c3)cc2)cc1 ; c1ccc(-c2ccccc2N(c2ccc(-c3ccc4ccccc4c3)cc2)c2cccc(-c3ccccc3-c3ccc4ccc5oc6ccccc6c5c4c3)c2)cc1] 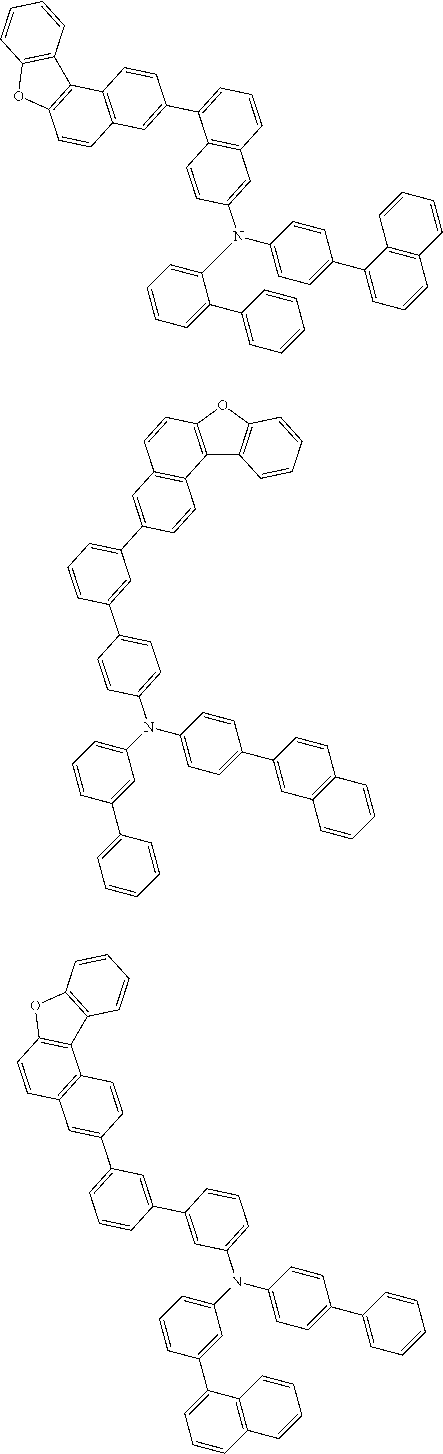 [SMILES: c1ccc(-c2ccccc2N(c2ccc(-c3cccc4ccccc34)cc2)c2ccc3c(-c4ccc5c(ccc6oc7ccccc7c65)c4)cccc3c2)cc1 ; c1ccc(-c2cccc(N(c3ccc(-c4cccc(-c5ccc6c(ccc7oc8ccccc8c76)c5)c4)cc3)c3ccc(-c4ccc5ccccc5c4)cc3)c2)cc1 ; c1ccc(-c2ccc(N(c3cccc(-c4cccc(-c5ccc6c(ccc7oc8ccccc8c76)c5)c4)c3)c3cccc(-c4cccc5ccccc45)c3)cc2)cc1] 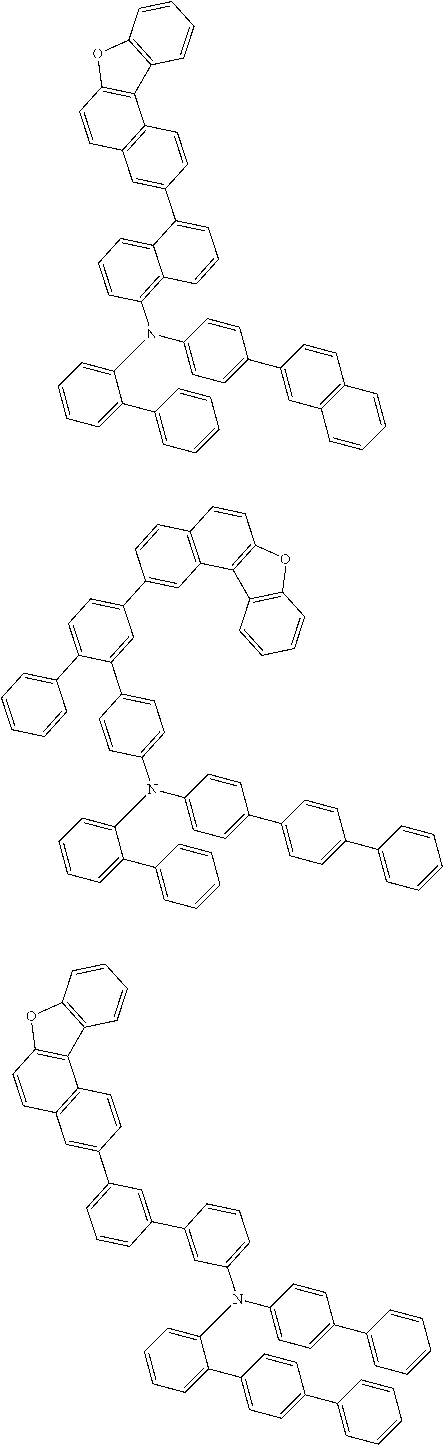 [SMILES: c1ccc(-c2ccccc2N(c2ccc(-c3ccc4ccccc4c3)cc2)c2cccc3c(-c4ccc5c(ccc6oc7ccccc7c65)c4)cccc23)cc1 ; c1ccc(-c2ccc(-c3ccc(N(c4ccc(-c5cc(-c6ccc7ccc8oc9ccccc9c8c7c6)ccc5-c5ccccc5)cc4)c4ccccc4-c4ccccc4)cc3)cc2)cc1 ; c1ccc(-c2ccc(-c3ccccc3N(c3ccc(-c4ccccc4)cc3)c3cccc(-c4cccc(-c5ccc6c(ccc7oc8ccccc8c76)c5)c4)c3)cc2)cc1] 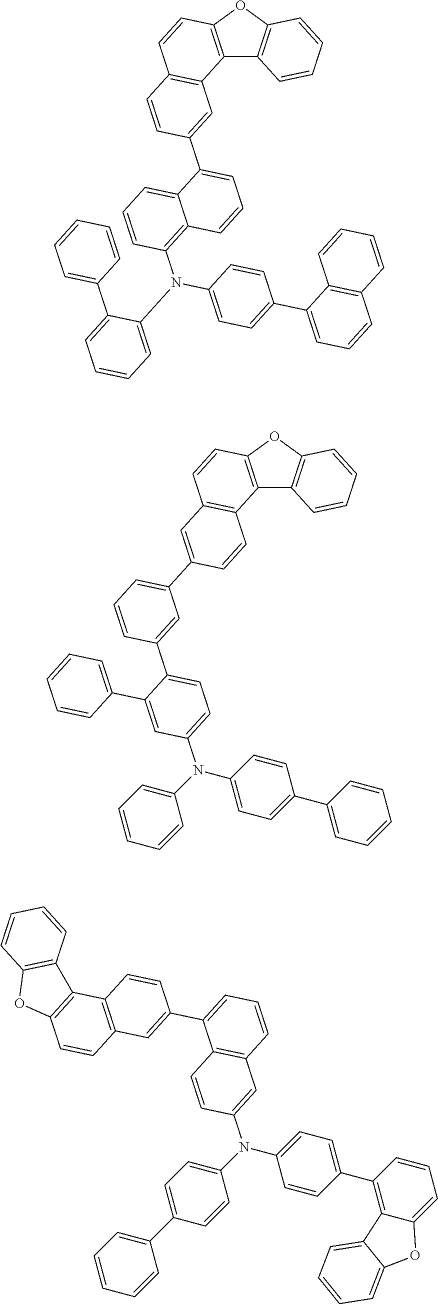 [SMILES: c1ccc(-c2ccccc2N(c2ccc(-c3cccc4ccccc34)cc2)c2cccc3c(-c4ccc5ccc6oc7ccccc7c6c5c4)cccc23)cc1 ; c1ccc(-c2ccc(N(c3ccccc3)c3ccc(-c4cccc(-c5ccc6c(ccc7oc8ccccc8c76)c5)c4)c(-c4ccccc4)c3)cc2)cc1 ; c1ccc(-c2ccc(N(c3ccc(-c4cccc5oc6ccccc6c45)cc3)c3ccc4c(-c5ccc6c(ccc7oc8ccccc8c76)c5)cccc4c3)cc2)cc1] 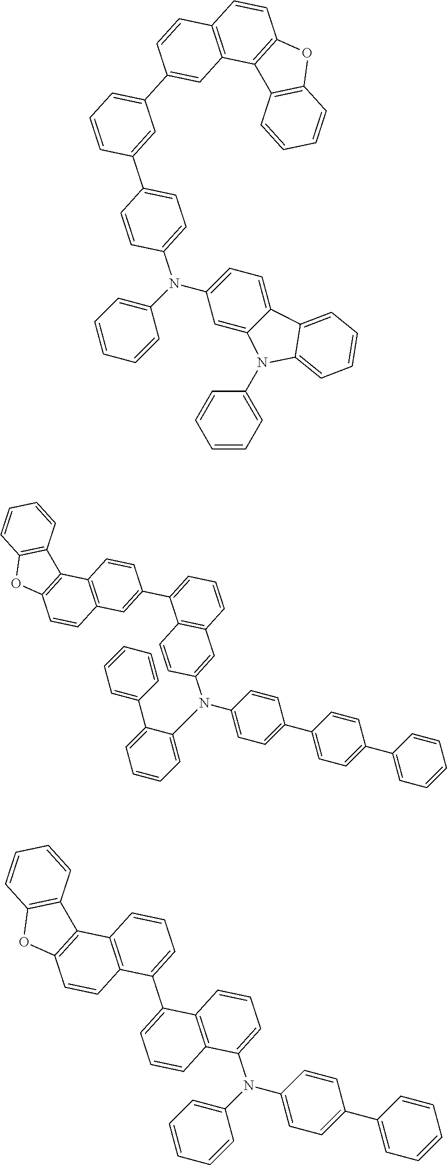 [SMILES: c1ccc(N(c2ccc(-c3cccc(-c4ccc5ccc6oc7ccccc7c6c5c4)c3)cc2)c2ccc3c4ccccc4n(-c4ccccc4)c3c2)cc1 ; c1ccc(-c2ccc(-c3ccc(N(c4ccc5c(-c6ccc7c(ccc8oc9ccccc9c87)c6)cccc5c4)c4ccccc4-c4ccccc4)cc3)cc2)cc1 ; c1ccc(-c2ccc(N(c3ccccc3)c3cccc4c(-c5cccc6c5ccc5oc7ccccc7c56)cccc34)cc2)cc1] 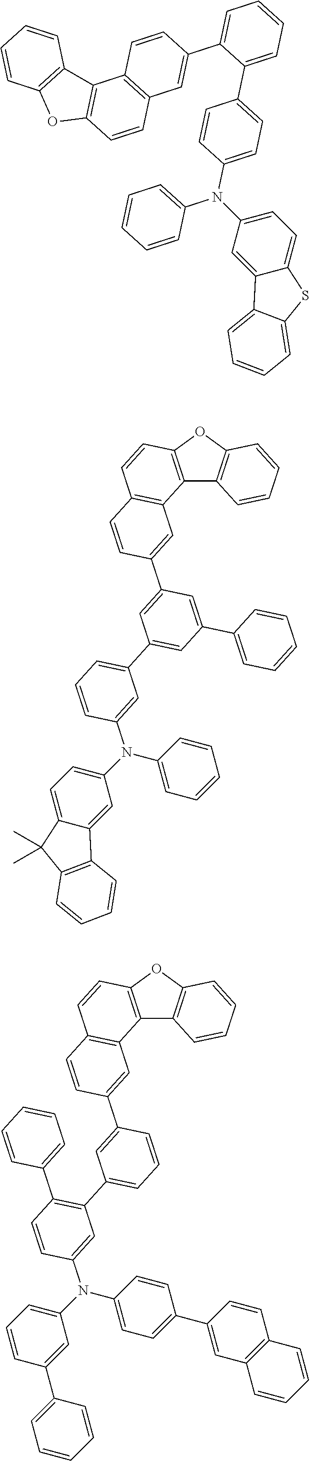 [SMILES: c1ccc(N(c2ccc(-c3ccccc3-c3ccc4c(ccc5oc6ccccc6c54)c3)cc2)c2ccc3sc4ccccc4c3c2)cc1 ; CC1(C)c2ccccc2-c2cc(N(c3ccccc3)c3cccc(-c4cc(-c5ccccc5)cc(-c5ccc6ccc7oc8ccccc8c7c6c5)c4)c3)ccc21 ; c1ccc(-c2cccc(N(c3ccc(-c4ccc5ccccc5c4)cc3)c3ccc(-c4ccccc4)c(-c4cccc(-c5ccc6ccc7oc8ccccc8c7c6c5)c4)c3)c2)cc1] 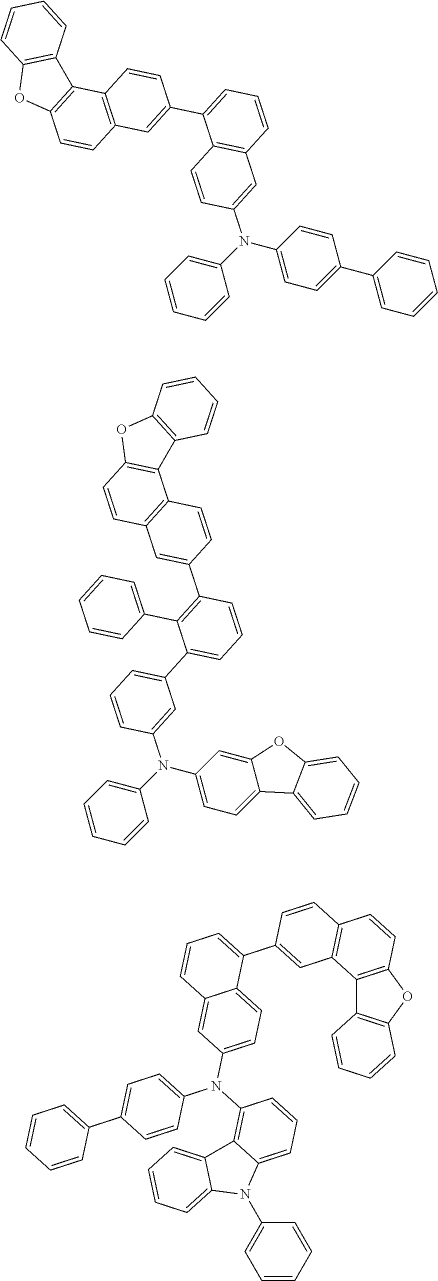 [SMILES: c1ccc(-c2ccc(N(c3ccccc3)c3ccc4c(-c5ccc6c(ccc7oc8ccccc8c76)c5)cccc4c3)cc2)cc1 ; c1ccc(-c2c(-c3cccc(N(c4ccccc4)c4ccc5c(c4)oc4ccccc45)c3)cccc2-c2ccc3c(ccc4oc5ccccc5c43)c2)cc1 ; c1ccc(-c2ccc(N(c3ccc4c(-c5ccc6ccc7oc8ccccc8c7c6c5)cccc4c3)c3cccc4c3c3ccccc3n4-c3ccccc3)cc2)cc1] 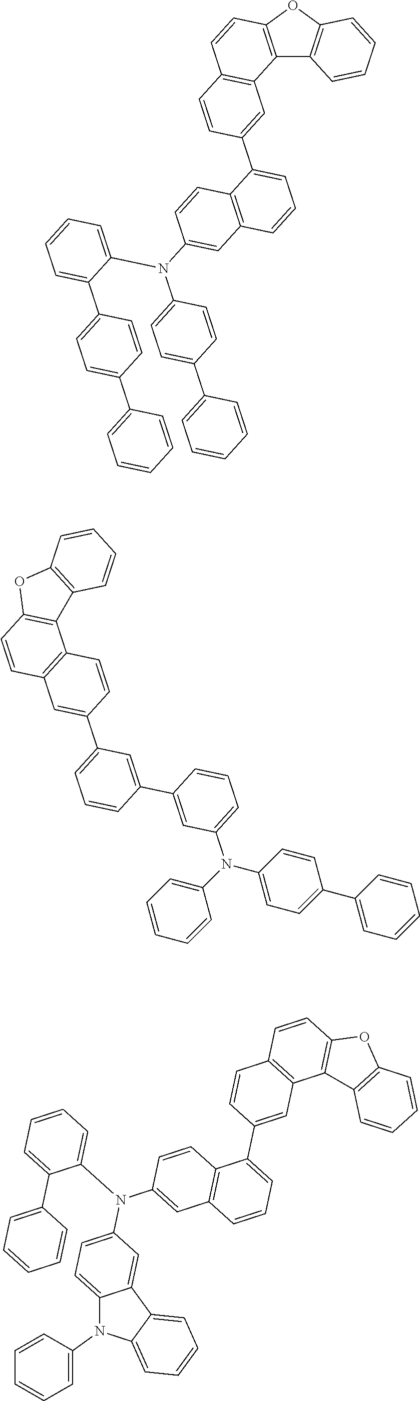 [SMILES: c1ccc(-c2ccc(-c3ccccc3N(c3ccc(-c4ccccc4)cc3)c3ccc4c(-c5ccc6ccc7oc8ccccc8c7c6c5)cccc4c3)cc2)cc1 ; c1ccc(-c2ccc(N(c3ccccc3)c3cccc(-c4cccc(-c5ccc6c(ccc7oc8ccccc8c76)c5)c4)c3)cc2)cc1 ; c1ccc(-c2ccccc2N(c2ccc3c(-c4ccc5ccc6oc7ccccc7c6c5c4)cccc3c2)c2ccc3c(c2)c2ccccc2n3-c2ccccc2)cc1] 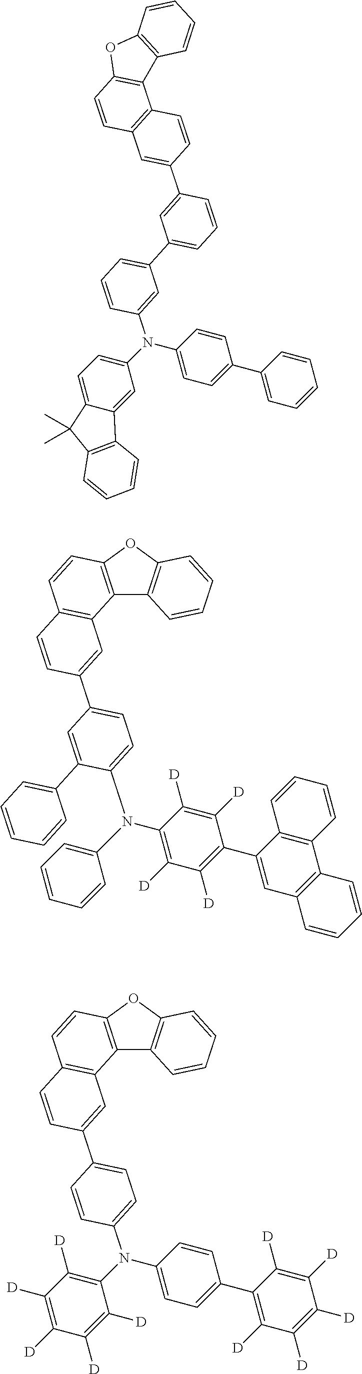 [SMILES: CC1(C)c2ccccc2-c2cc(N(c3ccc(-c4ccccc4)cc3)c3cccc(-c4cccc(-c5ccc6c(ccc7oc8ccccc8c76)c5)c4)c3)ccc21 ; [2H]c1c([2H])c(N(c2ccccc2)c2ccc(-c3ccc4ccc5oc6ccccc6c5c4c3)cc2-c2ccccc2)c([2H])c([2H])c1-c1cc2ccccc2c2ccccc12 ; [2H]c1c([2H])c([2H])c(-c2ccc(N(c3ccc(-c4ccc5ccc6oc7ccccc7c6c5c4)cc3)c3c([2H])c([2H])c([2H])c([2H])c3[2H])cc2)c([2H])c1[2H]] 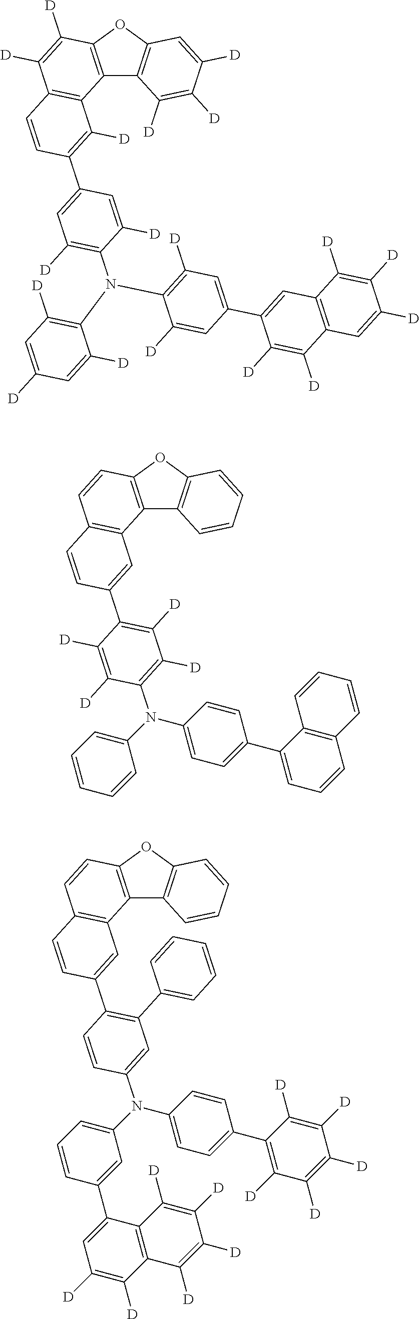 [SMILES: [2H]c1cc([2H])c(N(c2c([2H])cc(-c3cc4c([2H])c([2H])c([2H])cc4c([2H])c3[2H])cc2[2H])c2c([2H])cc(-c3ccc4c([2H])c([2H])c5oc6cc([2H])c([2H])c([2H])c6c5c4c3[2H])cc2[2H])c([2H])c1 ; [2H]c1c([2H])c(N(c2ccccc2)c2ccc(-c3cccc4ccccc34)cc2)c([2H])c([2H])c1-c1ccc2ccc3oc4ccccc4c3c2c1 ; [2H]c1cc(-c2cccc(N(c3ccc(-c4c([2H])c([2H])c([2H])c([2H])c4[2H])cc3)c3ccc(-c4ccc5ccc6oc7ccccc7c6c5c4)c(-c4ccccc4)c3)c2)c2c([2H])c([2H])c([2H])c([2H])c2c1[2H]] 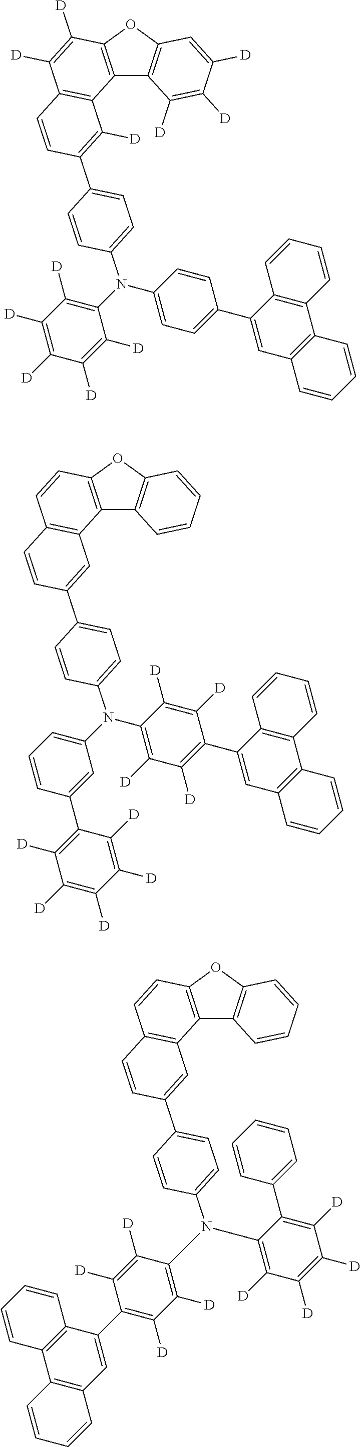 [SMILES: [2H]c1cc2oc3c([2H])c([2H])c4ccc(-c5ccc(N(c6ccc(-c7cc8ccccc8c8ccccc78)cc6)c6c([2H])c([2H])c([2H])c([2H])c6[2H])cc5)c([2H])c4c3c2c([2H])c1[2H] ; [2H]c1c([2H])c([2H])c(-c2cccc(N(c3ccc(-c4ccc5ccc6oc7ccccc7c6c5c4)cc3)c3c([2H])c([2H])c(-c4cc5ccccc5c5ccccc45)c([2H])c3[2H])c2)c([2H])c1[2H] ; [2H]c1c([2H])c([2H])c(N(c2ccc(-c3ccc4ccc5oc6ccccc6c5c4c3)cc2)c2c([2H])c([2H])c(-c3cc4ccccc4c4ccccc34)c([2H])c2[2H])c(-c2ccccc2)c1[2H]] 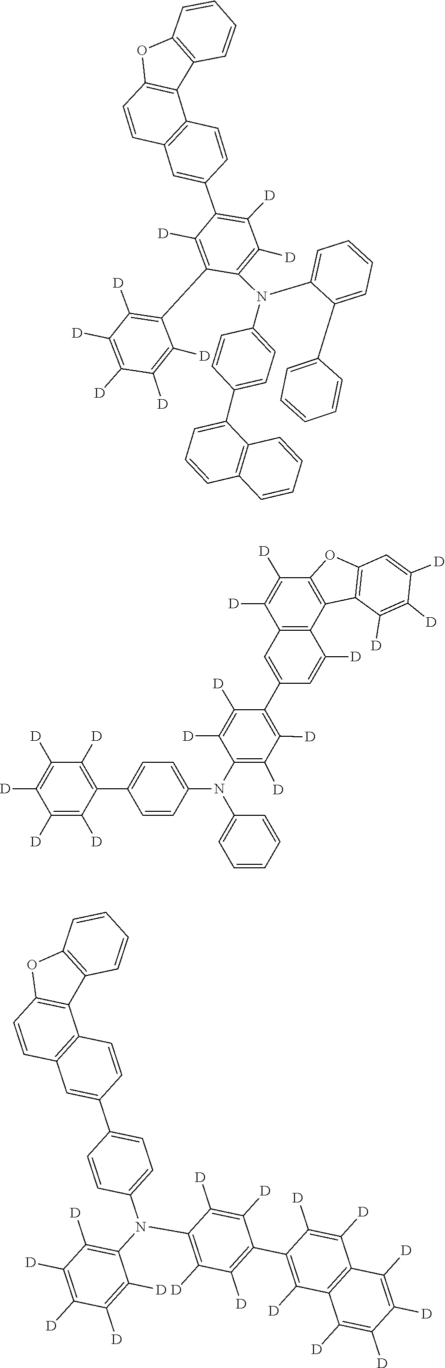 [SMILES: [2H]c1c([2H])c([2H])c(-c2c([2H])c(-c3ccc4c(ccc5oc6ccccc6c54)c3)c([2H])c([2H])c2N(c2ccc(-c3cccc4ccccc34)cc2)c2ccccc2-c2ccccc2)c([2H])c1[2H] ; [2H]c1cc2oc3c([2H])c([2H])c4cc(-c5c([2H])c([2H])c(N(c6ccccc6)c6ccc(-c7c([2H])c([2H])c([2H])c([2H])c7[2H])cc6)c([2H])c5[2H])cc([2H])c4c3c2c([2H])c1[2H] ; [2H]c1c([2H])c([2H])c(N(c2ccc(-c3ccc4c(ccc5oc6ccccc6c54)c3)cc2)c2c([2H])c([2H])c(-c3c([2H])c([2H])c4c([2H])c([2H])c([2H])c([2H])c4c3[2H])c([2H])c2[2H])c([2H])c1[2H]] 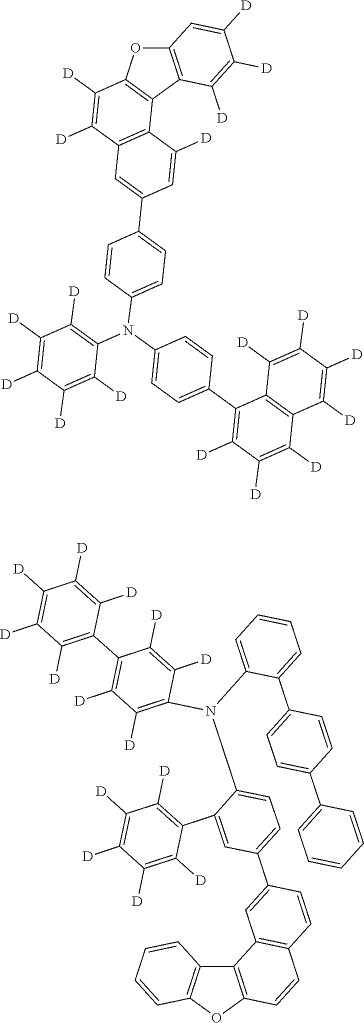 [SMILES: [2H]c1cc2oc3c([2H])c([2H])c4cc(-c5ccc(N(c6ccc(-c7c([2H])c([2H])c([2H])c8c([2H])c([2H])c([2H])c([2H])c78)cc6)c6c([2H])c([2H])c([2H])c([2H])c6[2H])cc5)cc([2H])c4c3c2c([2H])c1[2H] ; [2H]c1c([2H])c([2H])c(-c2cc(-c3ccc4ccc5oc6ccccc6c5c4c3)ccc2N(c2ccccc2-c2ccc(-c3ccccc3)cc2)c2c([2H])c([2H])c(-c3c([2H])c([2H])c([2H])c([2H])c3[2H])c([2H])c2[2H])c([2H])c1[2H]] 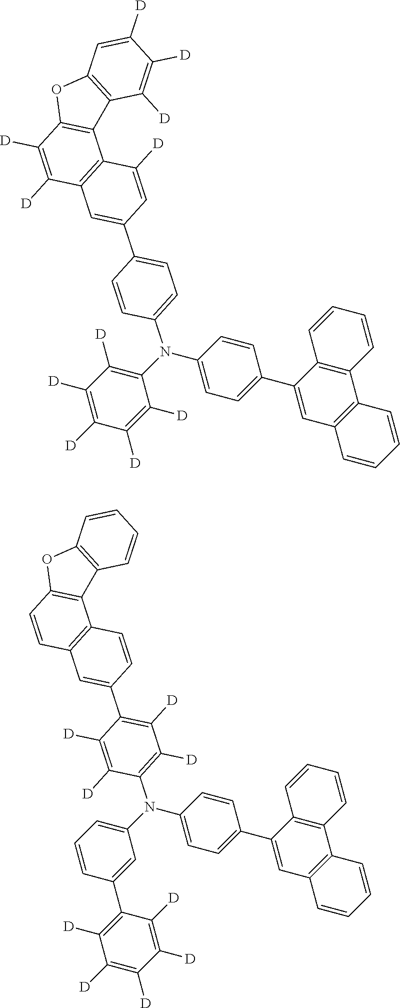 [SMILES: [2H]c1cc2oc3c([2H])c([2H])c4cc(-c5ccc(N(c6ccc(-c7cc8ccccc8c8ccccc78)cc6)c6c([2H])c([2H])c([2H])c([2H])c6[2H])cc5)cc([2H])c4c3c2c([2H])c1[2H] ; [2H]c1c([2H])c([2H])c(-c2cccc(N(c3ccc(-c4cc5ccccc5c5ccccc45)cc3)c3c([2H])c([2H])c(-c4ccc5c(ccc6oc7ccccc7c65)c4)c([2H])c3[2H])c2)c([2H])c1[2H]] 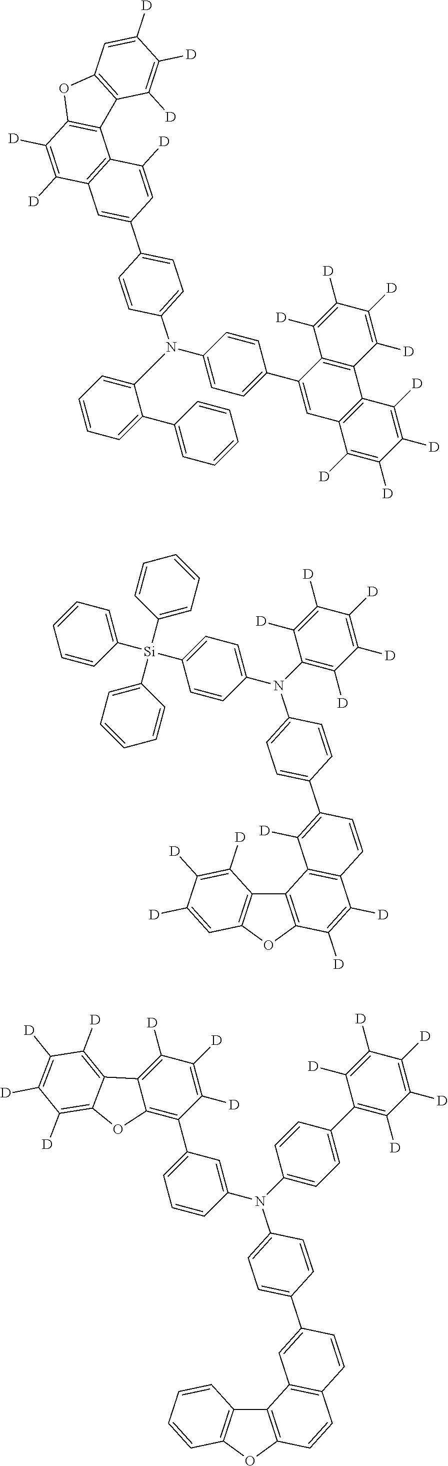 [SMILES: [2H]c1cc2oc3c([2H])c([2H])c4cc(-c5ccc(N(c6ccc(-c7cc8c([2H])c([2H])c([2H])c([2H])c8c8c([2H])c([2H])c([2H])c([2H])c78)cc6)c6ccccc6-c6ccccc6)cc5)cc([2H])c4c3c2c([2H])c1[2H] ; [2H]c1cc2oc3c([2H])c([2H])c4ccc(-c5ccc(N(c6ccc([Si](c7ccccc7)(c7ccccc7)c7ccccc7)cc6)c6c([2H])c([2H])c([2H])c([2H])c6[2H])cc5)c([2H])c4c3c2c([2H])c1[2H] ; [2H]c1c([2H])c([2H])c(-c2ccc(N(c3ccc(-c4ccc5ccc6oc7ccccc7c6c5c4)cc3)c3cccc(-c4c([2H])c([2H])c([2H])c5c4oc4c([2H])c([2H])c([2H])c([2H])c45)c3)cc2)c([2H])c1[2H]] 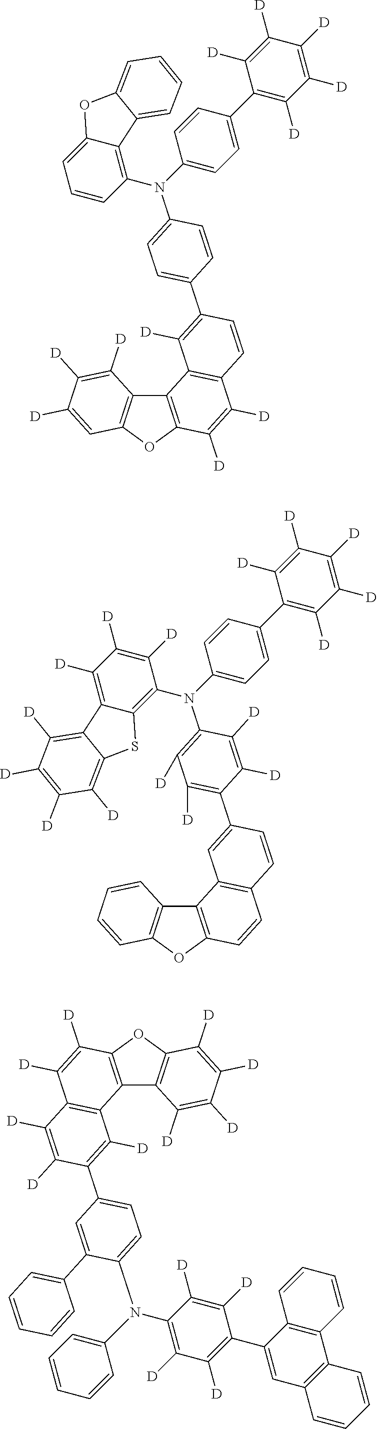 [SMILES: [2H]c1cc2oc3c([2H])c([2H])c4ccc(-c5ccc(N(c6ccc(-c7c([2H])c([2H])c([2H])c([2H])c7[2H])cc6)c6cccc7oc8ccccc8c67)cc5)c([2H])c4c3c2c([2H])c1[2H] ; [2H]c1c([2H])c([2H])c(-c2ccc(N(c3c([2H])c([2H])c(-c4ccc5ccc6oc7ccccc7c6c5c4)c([2H])c3[2H])c3c([2H])c([2H])c([2H])c4c3sc3c([2H])c([2H])c([2H])c([2H])c34)cc2)c([2H])c1[2H] ; [2H]c1c([2H])c(N(c2ccccc2)c2ccc(-c3c([2H])c([2H])c4c([2H])c([2H])c5oc6c([2H])c([2H])c([2H])c([2H])c6c5c4c3[2H])cc2-c2ccccc2)c([2H])c([2H])c1-c1cc2ccccc2c2ccccc12] 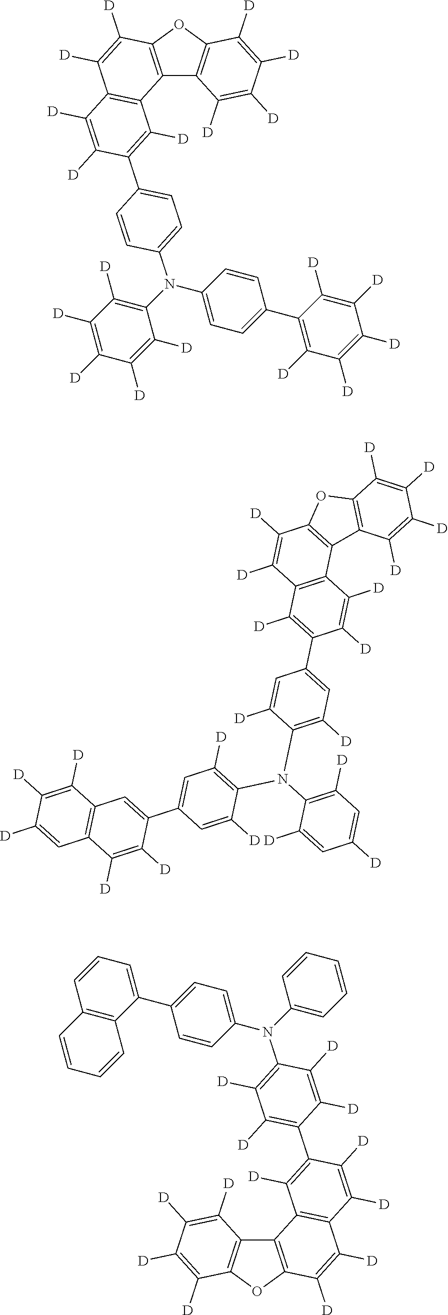 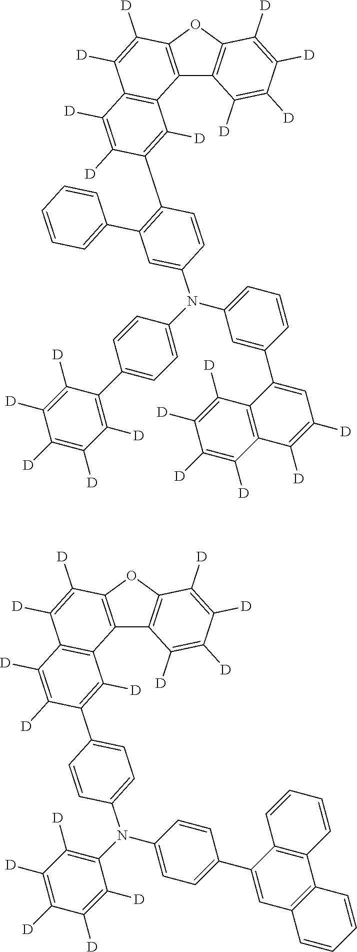 [SMILES: [2H]c1cc(-c2cccc(N(c3ccc(-c4c([2H])c([2H])c([2H])c([2H])c4[2H])cc3)c3ccc(-c4c([2H])c([2H])c5c([2H])c([2H])c6oc7c([2H])c([2H])c([2H])c([2H])c7c6c5c4[2H])c(-c4ccccc4)c3)c2)c2c([2H])c([2H])c([2H])c([2H])c2c1[2H] ; [2H]c1c([2H])c([2H])c(N(c2ccc(-c3c([2H])c([2H])c4c([2H])c([2H])c5oc6c([2H])c([2H])c([2H])c([2H])c6c5c4c3[2H])cc2)c2ccc(-c3cc4ccccc4c4ccccc34)cc2)c([2H])c1[2H]] 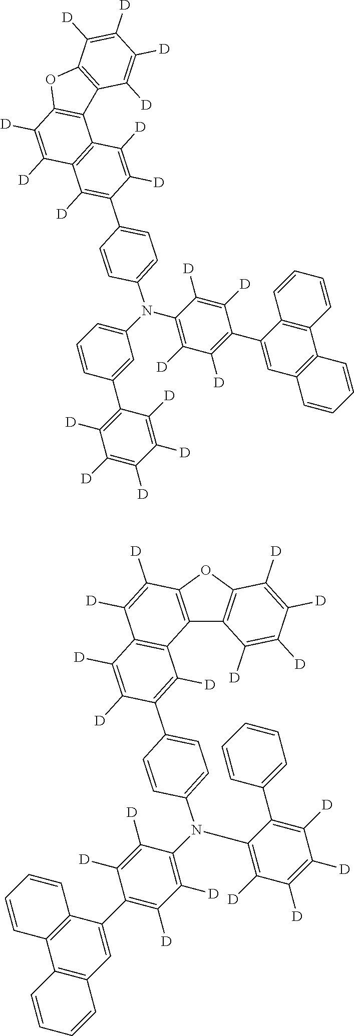 [SMILES: [2H]c1c([2H])c([2H])c(-c2cccc(N(c3ccc(-c4c([2H])c([2H])c5c(c4[2H])c([2H])c([2H])c4oc6c([2H])c([2H])c([2H])c([2H])c6c45)cc3)c3c([2H])c([2H])c(-c4cc5ccccc5c5ccccc45)c([2H])c3[2H])c2)c([2H])c1[2H] ; [2H]c1c([2H])c([2H])c(N(c2ccc(-c3c([2H])c([2H])c4c([2H])c([2H])c5oc6c([2H])c([2H])c([2H])c([2H])c6c5c4c3[2H])cc2)c2c([2H])c([2H])c(-c3cc4ccccc4c4ccccc34)c([2H])c2[2H])c(-c2ccccc2)c1[2H]] 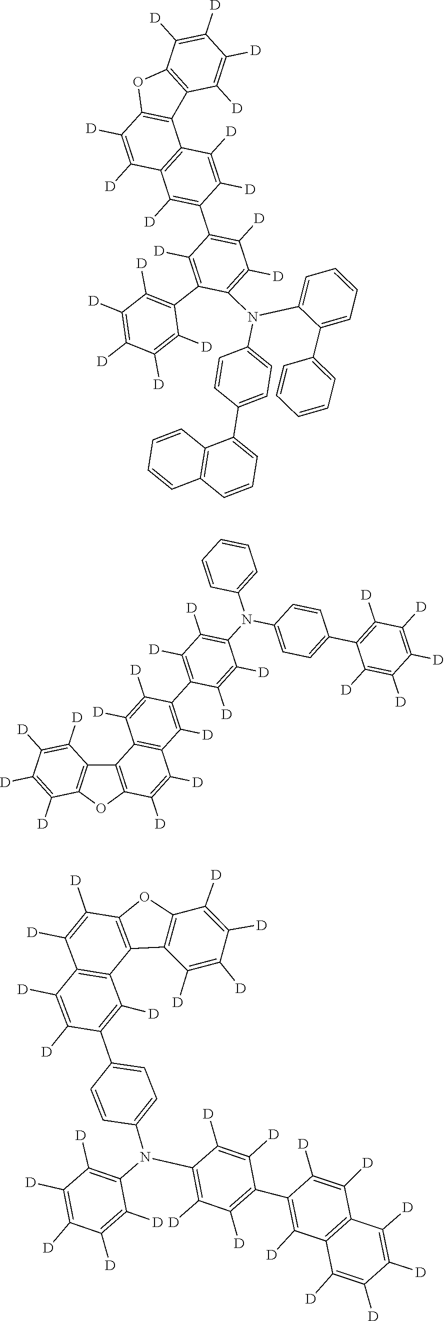 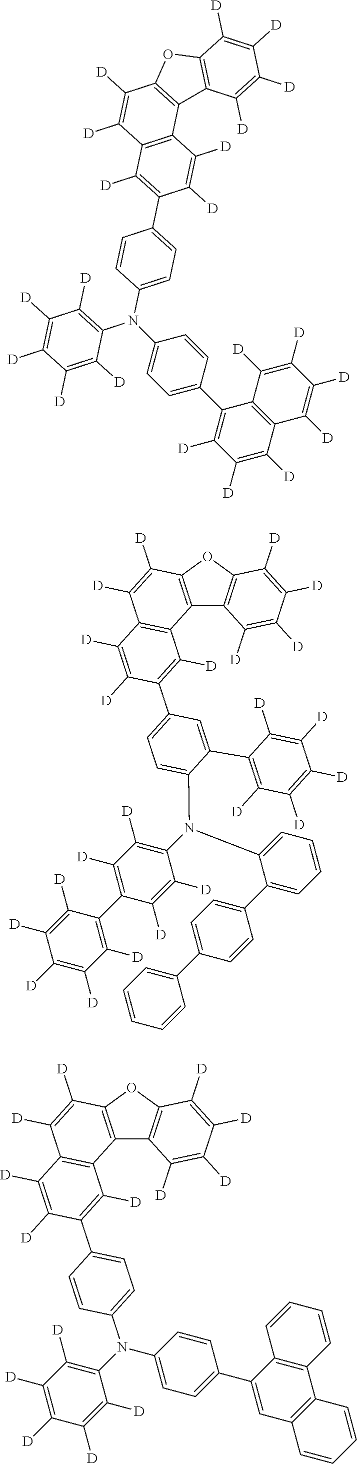 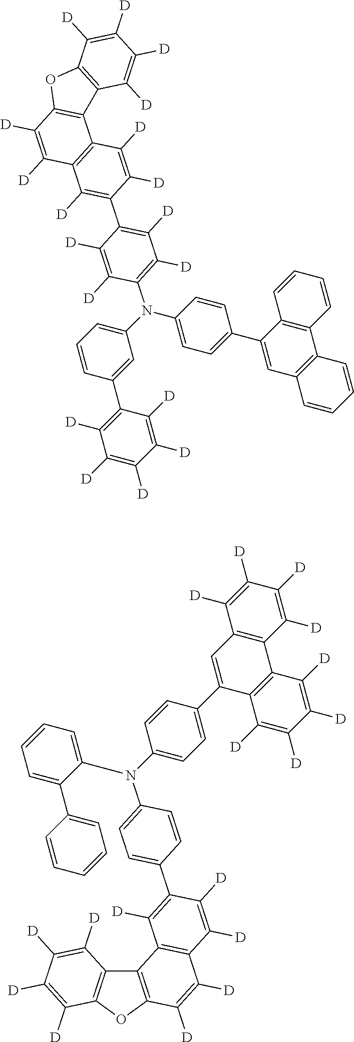 [SMILES: [2H]c1c([2H])c([2H])c(-c2cccc(N(c3ccc(-c4cc5ccccc5c5ccccc45)cc3)c3c([2H])c([2H])c(-c4c([2H])c([2H])c5c(c4[2H])c([2H])c([2H])c4oc6c([2H])c([2H])c([2H])c([2H])c6c45)c([2H])c3[2H])c2)c([2H])c1[2H] ; [2H]c1c([2H])c([2H])c2c(cc(-c3ccc(N(c4ccc(-c5c([2H])c([2H])c6c([2H])c([2H])c7oc8c([2H])c([2H])c([2H])c([2H])c8c7c6c5[2H])cc4)c4ccccc4-c4ccccc4)cc3)c3c([2H])c([2H])c([2H])c([2H])c32)c1[2H]] 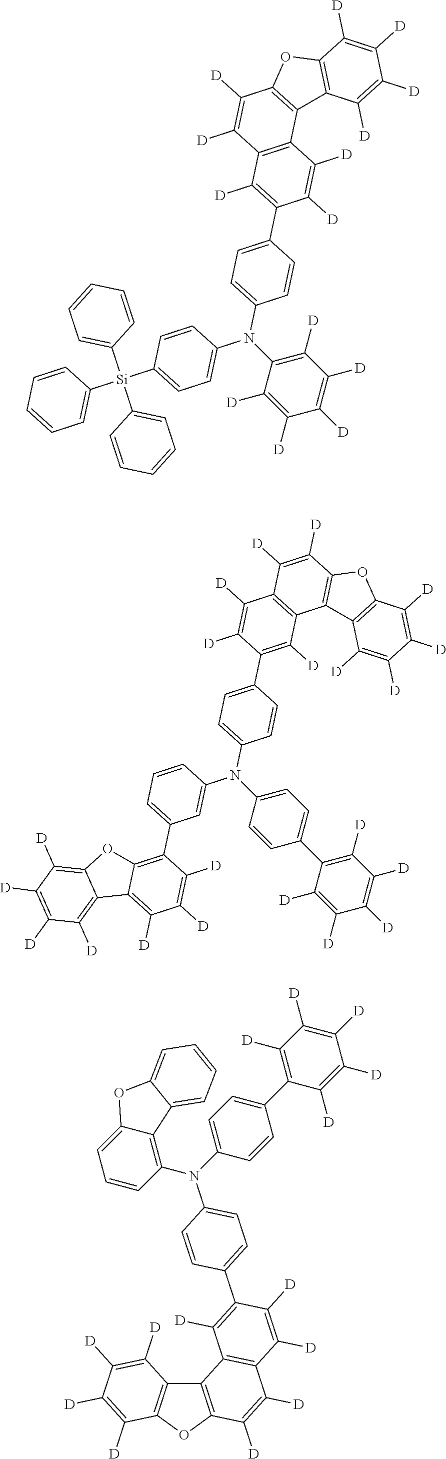 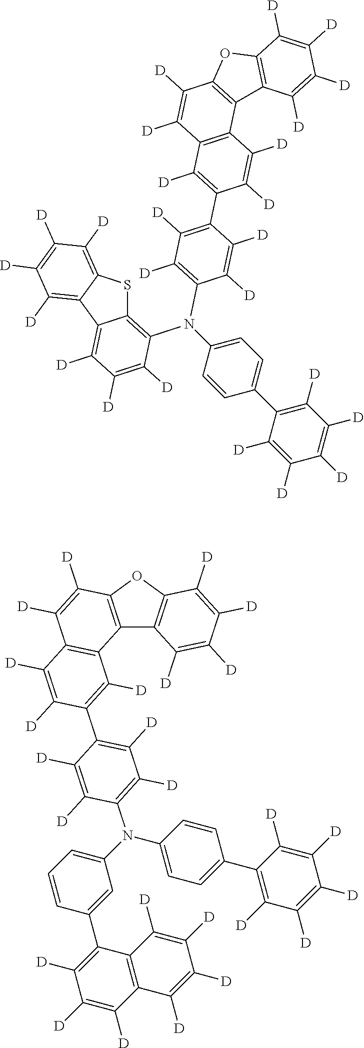 [SMILES: [2H]c1c([2H])c([2H])c(-c2ccc(N(c3c([2H])c([2H])c(-c4c([2H])c([2H])c5c(c4[2H])c([2H])c([2H])c4oc6c([2H])c([2H])c([2H])c([2H])c6c45)c([2H])c3[2H])c3c([2H])c([2H])c([2H])c4c3sc3c([2H])c([2H])c([2H])c([2H])c34)cc2)c([2H])c1[2H] ; [2H]c1c([2H])c([2H])c(-c2ccc(N(c3cccc(-c4c([2H])c([2H])c([2H])c5c([2H])c([2H])c([2H])c([2H])c45)c3)c3c([2H])c([2H])c(-c4c([2H])c([2H])c5c([2H])c([2H])c6oc7c([2H])c([2H])c([2H])c([2H])c7c6c5c4[2H])c([2H])c3[2H])cc2)c([2H])c1[2H]] 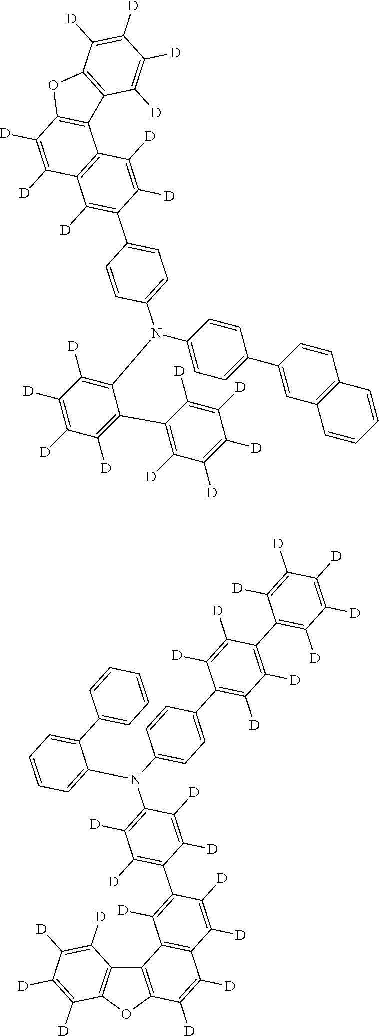 [SMILES: [2H]c1c([2H])c([2H])c(-c2c([2H])c([2H])c([2H])c([2H])c2N(c2ccc(-c3ccc4ccccc4c3)cc2)c2ccc(-c3c([2H])c([2H])c4c(c3[2H])c([2H])c([2H])c3oc5c([2H])c([2H])c([2H])c([2H])c5c34)cc2)c([2H])c1[2H] ; [2H]c1c([2H])c([2H])c(-c2c([2H])c([2H])c(-c3ccc(N(c4ccccc4-c4ccccc4)c4c([2H])c([2H])c(-c5c([2H])c([2H])c6c([2H])c([2H])c7oc8c([2H])c([2H])c([2H])c([2H])c8c7c6c5[2H])c([2H])c4[2H])cc3)c([2H])c2[2H])c([2H])c1[2H]] 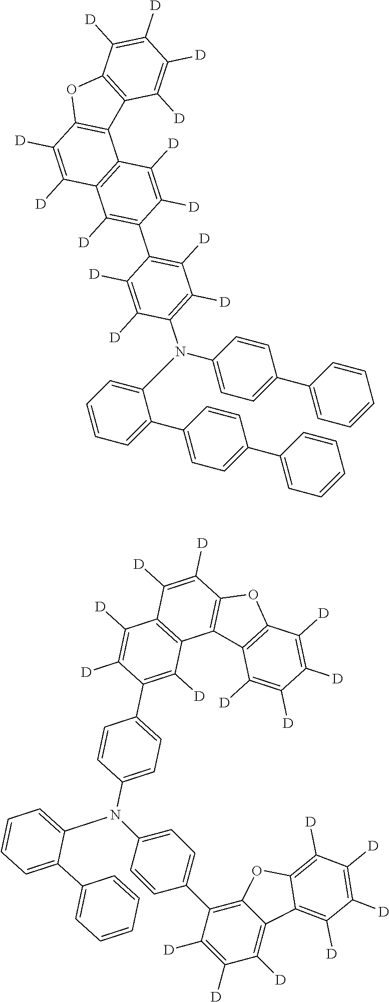 [SMILES: [2H]c1c([2H])c(N(c2ccc(-c3ccccc3)cc2)c2ccccc2-c2ccc(-c3ccccc3)cc2)c([2H])c([2H])c1-c1c([2H])c([2H])c2c(c1[2H])c([2H])c([2H])c1oc3c([2H])c([2H])c([2H])c([2H])c3c12 ; [2H]c1c([2H])c([2H])c2c(oc3c(-c4ccc(N(c5ccc(-c6c([2H])c([2H])c7c([2H])c([2H])c8oc9c([2H])c([2H])c([2H])c([2H])c9c8c7c6[2H])cc5)c5ccccc5-c5ccccc5)cc4)c([2H])c([2H])c([2H])c32)c1[2H]] 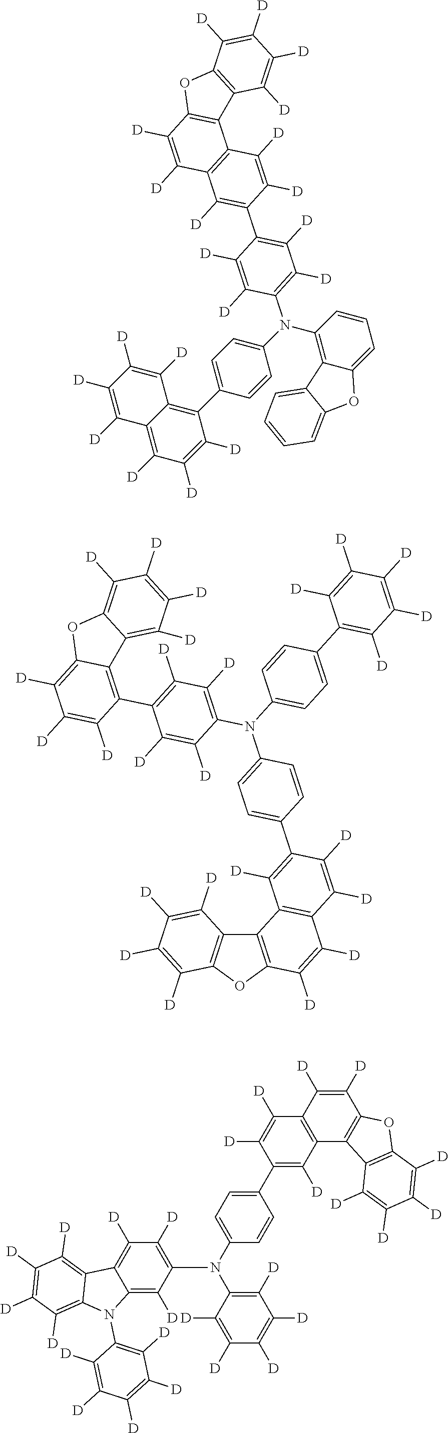 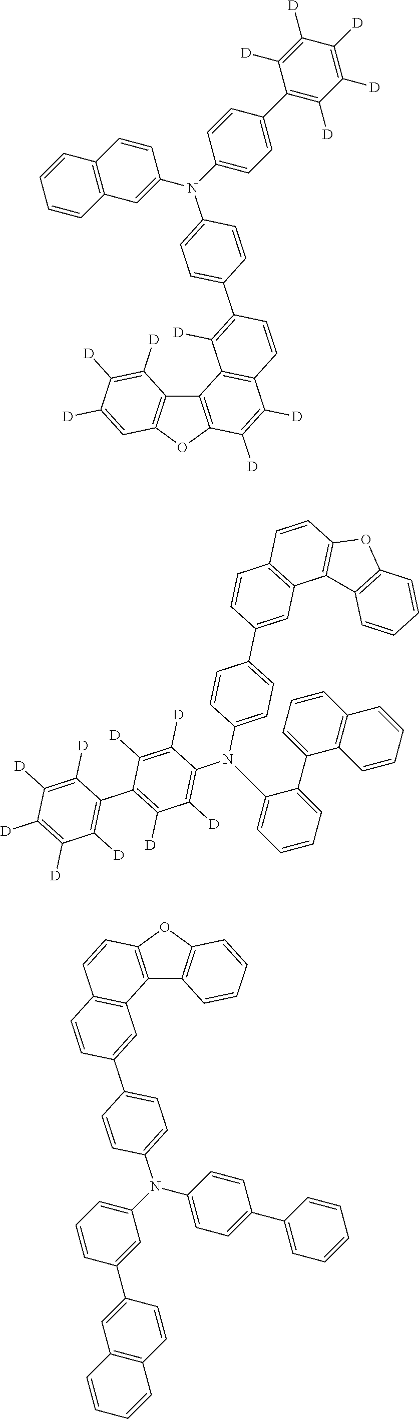 [SMILES: [2H]c1cc2oc3c([2H])c([2H])c4ccc(-c5ccc(N(c6ccc(-c7c([2H])c([2H])c([2H])c([2H])c7[2H])cc6)c6ccc7ccccc7c6)cc5)c([2H])c4c3c2c([2H])c1[2H] ; [2H]c1c([2H])c([2H])c(-c2c([2H])c([2H])c(N(c3ccc(-c4ccc5ccc6oc7ccccc7c6c5c4)cc3)c3ccccc3-c3cccc4ccccc34)c([2H])c2[2H])c([2H])c1[2H] ; c1ccc(-c2ccc(N(c3ccc(-c4ccc5ccc6oc7ccccc7c6c5c4)cc3)c3cccc(-c4ccc5ccccc5c4)c3)cc2)cc1] 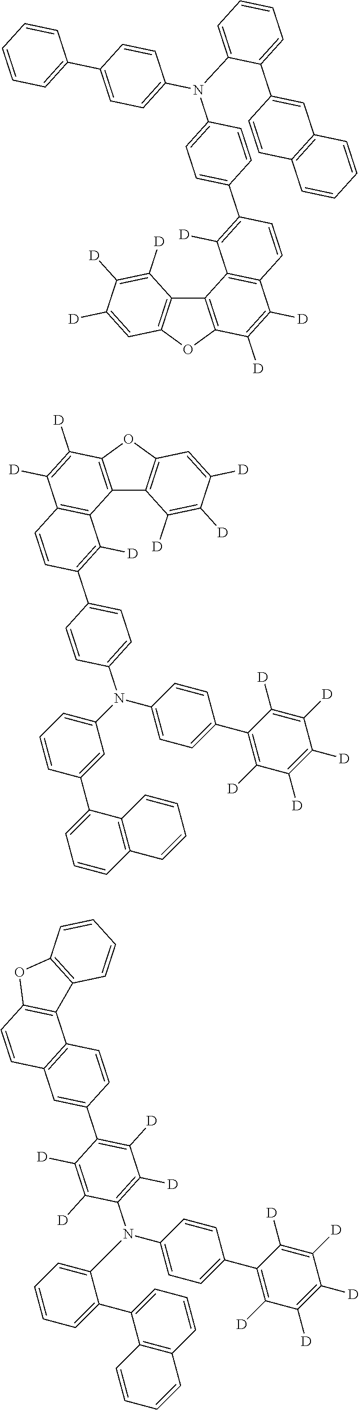 [SMILES: [2H]c1cc2oc3c([2H])c([2H])c4ccc(-c5ccc(N(c6ccc(-c7ccccc7)cc6)c6ccccc6-c6ccc7ccccc7c6)cc5)c([2H])c4c3c2c([2H])c1[2H] ; [2H]c1cc2oc3c([2H])c([2H])c4ccc(-c5ccc(N(c6ccc(-c7c([2H])c([2H])c([2H])c([2H])c7[2H])cc6)c6cccc(-c7cccc8ccccc78)c6)cc5)c([2H])c4c3c2c([2H])c1[2H] ; [2H]c1c([2H])c([2H])c(-c2ccc(N(c3ccccc3-c3cccc4ccccc34)c3c([2H])c([2H])c(-c4ccc5c(ccc6oc7ccccc7c65)c4)c([2H])c3[2H])cc2)c([2H])c1[2H]] 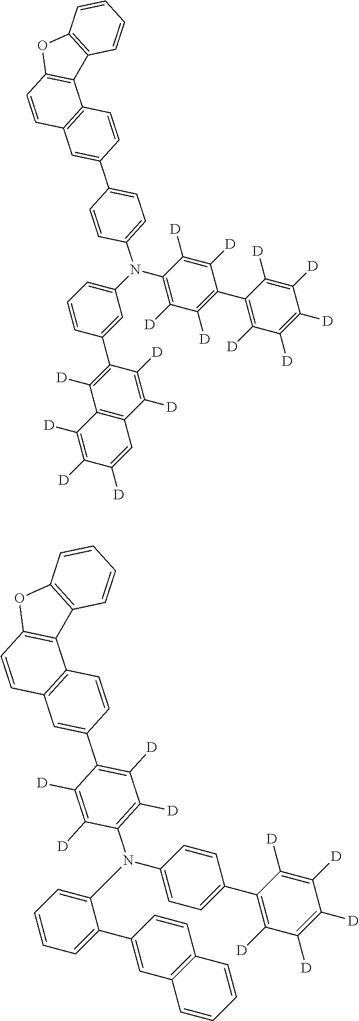 [SMILES: [2H]c1cc2c([2H])c([2H])c(-c3cccc(N(c4ccc(-c5ccc6c(ccc7oc8ccccc8c76)c5)cc4)c4c([2H])c([2H])c(-c5c([2H])c([2H])c([2H])c([2H])c5[2H])c([2H])c4[2H])c3)c([2H])c2c([2H])c1[2H] ; [2H]c1c([2H])c([2H])c(-c2ccc(N(c3ccccc3-c3ccc4ccccc4c3)c3c([2H])c([2H])c(-c4ccc5c(ccc6oc7ccccc7c65)c4)c([2H])c3[2H])cc2)c([2H])c1[2H]] 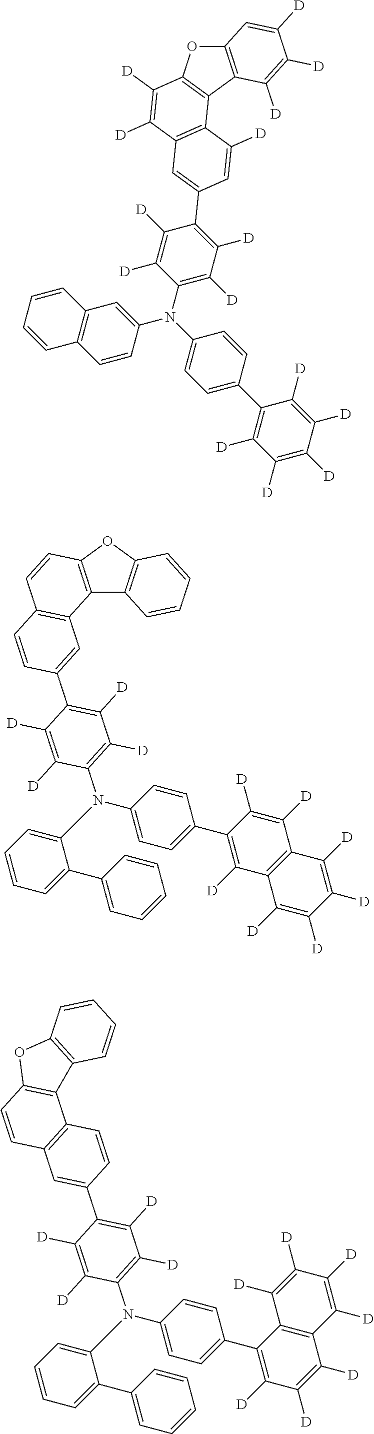 [SMILES: [2H]c1cc2oc3c([2H])c([2H])c4cc(-c5c([2H])c([2H])c(N(c6ccc(-c7c([2H])c([2H])c([2H])c([2H])c7[2H])cc6)c6ccc7ccccc7c6)c([2H])c5[2H])cc([2H])c4c3c2c([2H])c1[2H] ; [2H]c1c([2H])c(N(c2ccc(-c3c([2H])c([2H])c4c([2H])c([2H])c([2H])c([2H])c4c3[2H])cc2)c2ccccc2-c2ccccc2)c([2H])c([2H])c1-c1ccc2ccc3oc4ccccc4c3c2c1 ; [2H]c1c([2H])c(N(c2ccc(-c3c([2H])c([2H])c([2H])c4c([2H])c([2H])c([2H])c([2H])c34)cc2)c2ccccc2-c2ccccc2)c([2H])c([2H])c1-c1ccc2c(ccc3oc4ccccc4c32)c1] 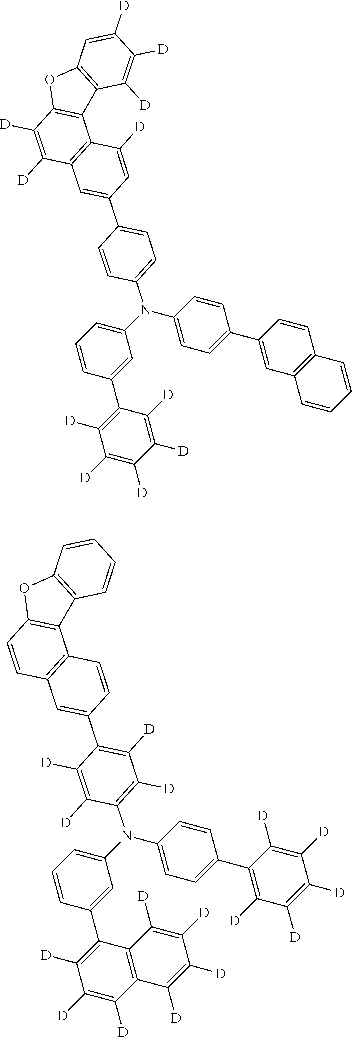 [SMILES: [2H]c1cc2oc3c([2H])c([2H])c4cc(-c5ccc(N(c6ccc(-c7ccc8ccccc8c7)cc6)c6cccc(-c7c([2H])c([2H])c([2H])c([2H])c7[2H])c6)cc5)cc([2H])c4c3c2c([2H])c1[2H] ; [2H]c1c([2H])c([2H])c(-c2ccc(N(c3cccc(-c4c([2H])c([2H])c([2H])c5c([2H])c([2H])c([2H])c([2H])c45)c3)c3c([2H])c([2H])c(-c4ccc5c(ccc6oc7ccccc7c65)c4)c([2H])c3[2H])cc2)c([2H])c1[2H]] 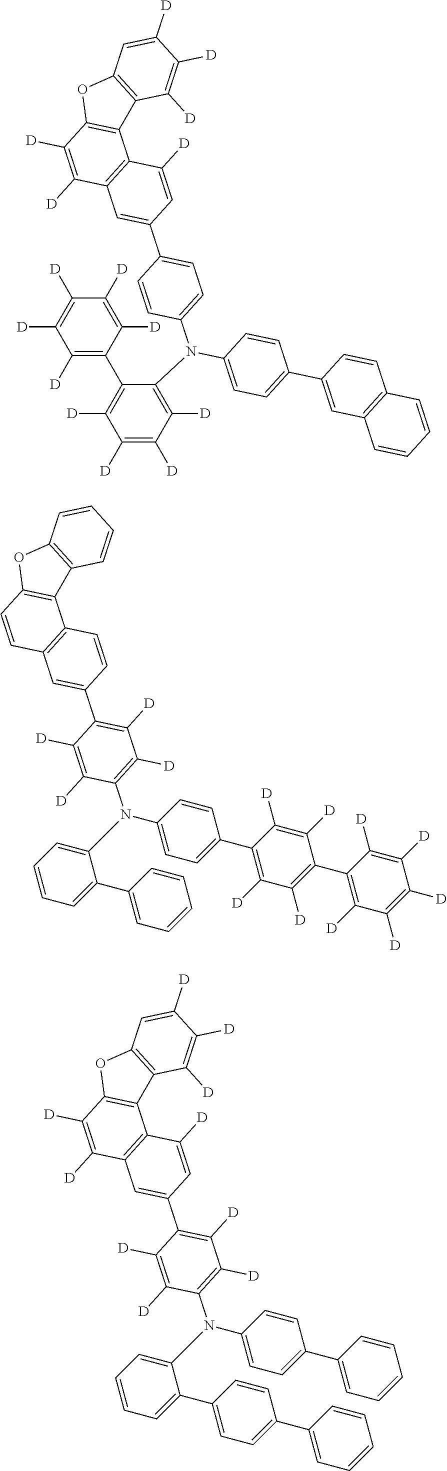 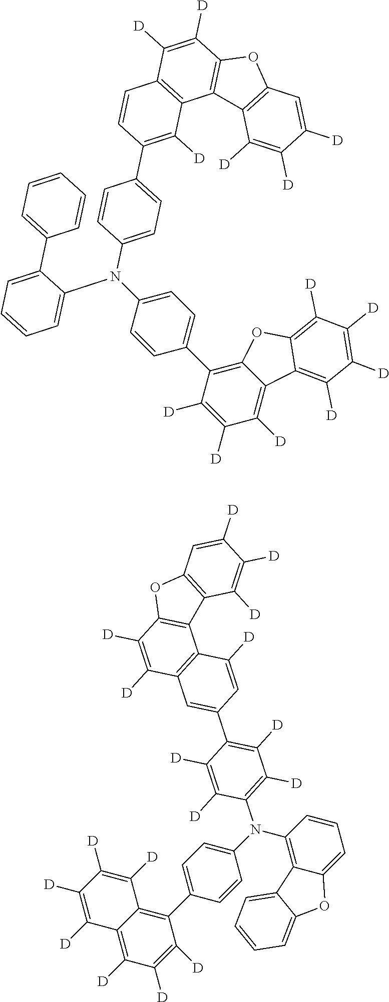 [SMILES: [2H]c1cc2oc3c([2H])c([2H])c4ccc(-c5ccc(N(c6ccc(-c7c([2H])c([2H])c([2H])c8c7oc7c([2H])c([2H])c([2H])c([2H])c78)cc6)c6ccccc6-c6ccccc6)cc5)c([2H])c4c3c2c([2H])c1[2H] ; [2H]c1cc2oc3c([2H])c([2H])c4cc(-c5c([2H])c([2H])c(N(c6ccc(-c7c([2H])c([2H])c([2H])c8c([2H])c([2H])c([2H])c([2H])c78)cc6)c6cccc7oc8ccccc8c67)c([2H])c5[2H])cc([2H])c4c3c2c([2H])c1[2H]] 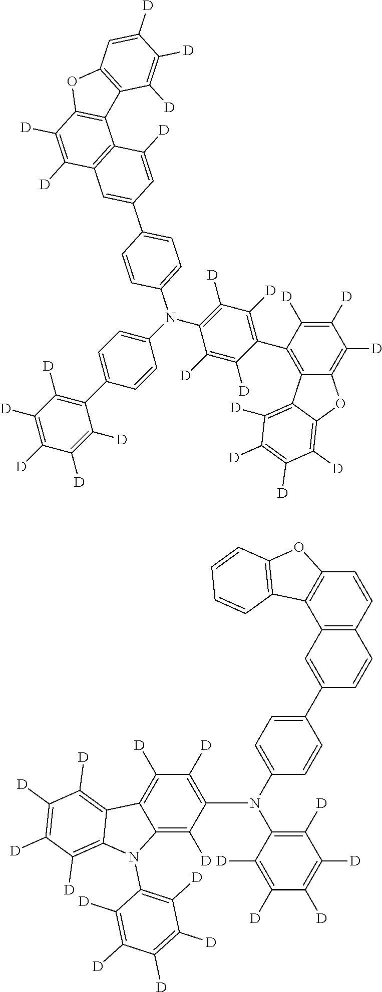 [SMILES: [2H]c1cc2oc3c([2H])c([2H])c4cc(-c5ccc(N(c6ccc(-c7c([2H])c([2H])c([2H])c([2H])c7[2H])cc6)c6c([2H])c([2H])c(-c7c([2H])c([2H])c([2H])c8oc9c([2H])c([2H])c([2H])c([2H])c9c78)c([2H])c6[2H])cc5)cc([2H])c4c3c2c([2H])c1[2H] ; [2H]c1c([2H])c([2H])c(N(c2ccc(-c3ccc4ccc5oc6ccccc6c5c4c3)cc2)c2c([2H])c([2H])c3c4c([2H])c([2H])c([2H])c([2H])c4n(-c4c([2H])c([2H])c([2H])c([2H])c4[2H])c3c2[2H])c([2H])c1[2H]] 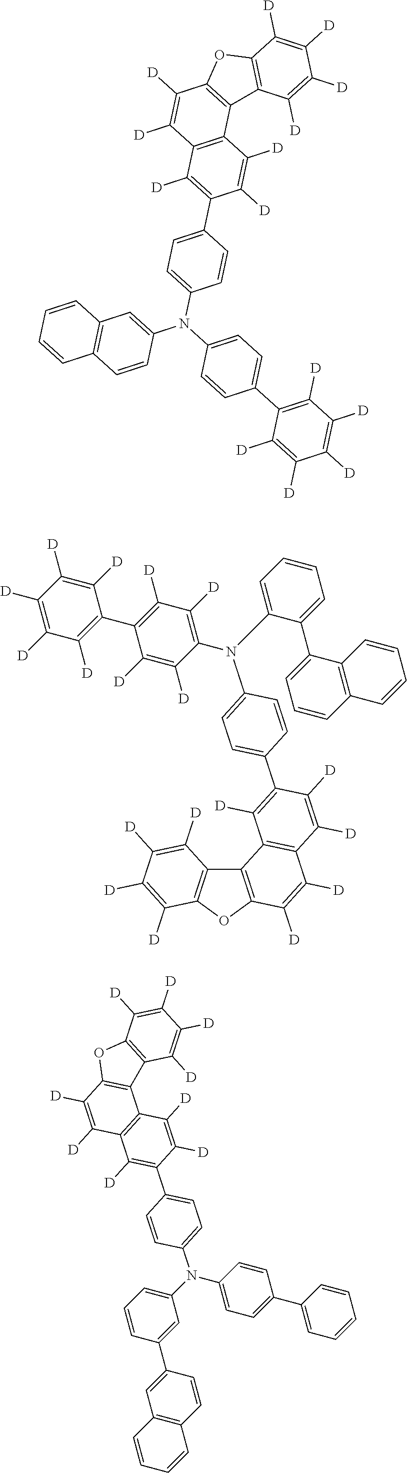 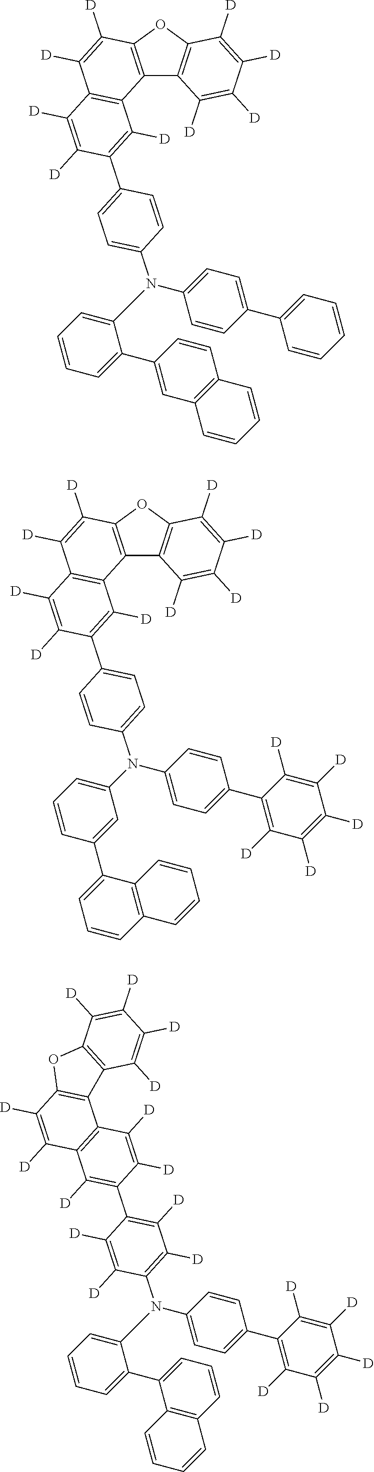 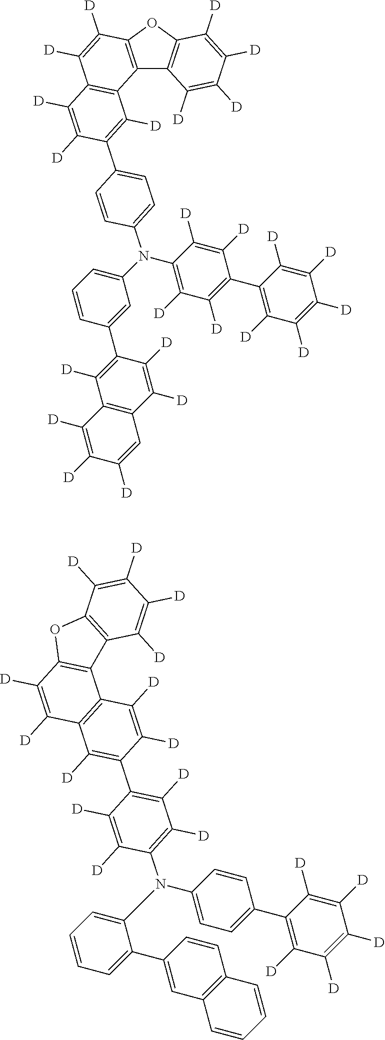 [SMILES: [2H]c1cc2c([2H])c([2H])c(-c3cccc(N(c4ccc(-c5c([2H])c([2H])c6c([2H])c([2H])c7oc8c([2H])c([2H])c([2H])c([2H])c8c7c6c5[2H])cc4)c4c([2H])c([2H])c(-c5c([2H])c([2H])c([2H])c([2H])c5[2H])c([2H])c4[2H])c3)c([2H])c2c([2H])c1[2H] ; [2H]c1c([2H])c([2H])c(-c2ccc(N(c3ccccc3-c3ccc4ccccc4c3)c3c([2H])c([2H])c(-c4c([2H])c([2H])c5c(c4[2H])c([2H])c([2H])c4oc6c([2H])c([2H])c([2H])c([2H])c6c45)c([2H])c3[2H])cc2)c([2H])c1[2H]] 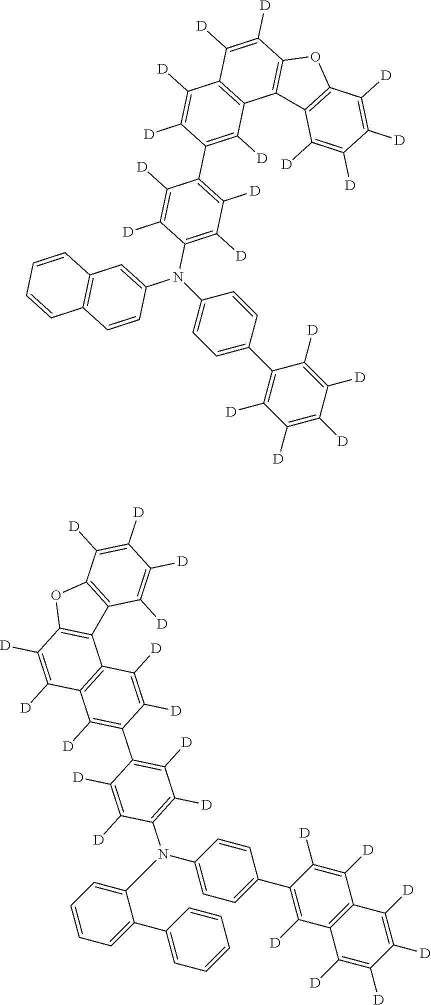 [SMILES: [2H]c1c([2H])c([2H])c(-c2ccc(N(c3ccc4ccccc4c3)c3c([2H])c([2H])c(-c4c([2H])c([2H])c5c([2H])c([2H])c6oc7c([2H])c([2H])c([2H])c([2H])c7c6c5c4[2H])c([2H])c3[2H])cc2)c([2H])c1[2H] ; [2H]c1c([2H])c(N(c2ccc(-c3c([2H])c([2H])c4c([2H])c([2H])c([2H])c([2H])c4c3[2H])cc2)c2ccccc2-c2ccccc2)c([2H])c([2H])c1-c1c([2H])c([2H])c2c(c1[2H])c([2H])c([2H])c1oc3c([2H])c([2H])c([2H])c([2H])c3c12] 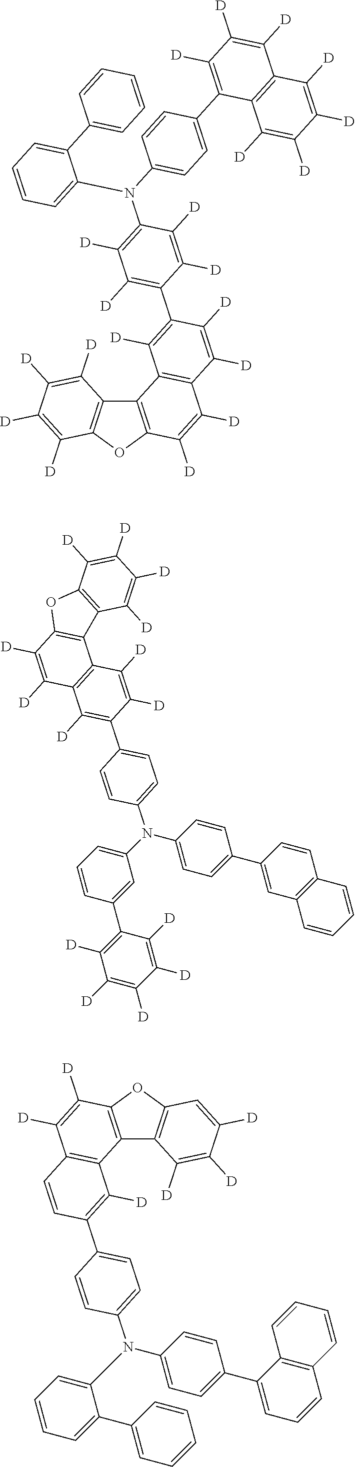 [SMILES: [2H]c1c([2H])c(N(c2ccc(-c3c([2H])c([2H])c([2H])c4c([2H])c([2H])c([2H])c([2H])c34)cc2)c2ccccc2-c2ccccc2)c([2H])c([2H])c1-c1c([2H])c([2H])c2c([2H])c([2H])c3oc4c([2H])c([2H])c([2H])c([2H])c4c3c2c1[2H] ; [2H]c1c([2H])c([2H])c(-c2cccc(N(c3ccc(-c4ccc5ccccc5c4)cc3)c3ccc(-c4c([2H])c([2H])c5c(c4[2H])c([2H])c([2H])c4oc6c([2H])c([2H])c([2H])c([2H])c6c45)cc3)c2)c([2H])c1[2H] ; [2H]c1cc2oc3c([2H])c([2H])c4ccc(-c5ccc(N(c6ccc(-c7cccc8ccccc78)cc6)c6ccccc6-c6ccccc6)cc5)c([2H])c4c3c2c([2H])c1[2H]] 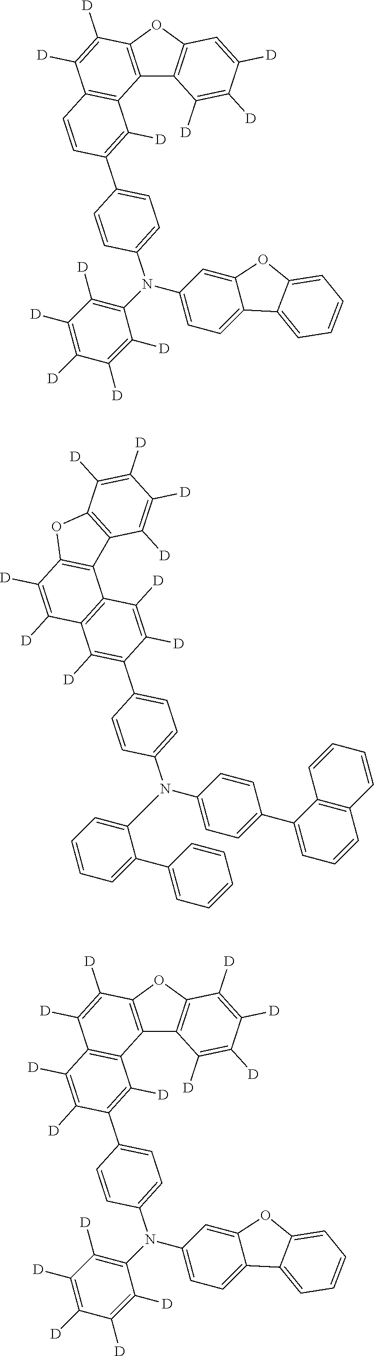 [SMILES: [2H]c1cc2oc3c([2H])c([2H])c4ccc(-c5ccc(N(c6ccc7c(c6)oc6ccccc67)c6c([2H])c([2H])c([2H])c([2H])c6[2H])cc5)c([2H])c4c3c2c([2H])c1[2H] ; [2H]c1c([2H])c([2H])c2c(oc3c([2H])c([2H])c4c([2H])c(-c5ccc(N(c6ccc(-c7cccc8ccccc78)cc6)c6ccccc6-c6ccccc6)cc5)c([2H])c([2H])c4c32)c1[2H] ; [2H]c1c([2H])c([2H])c(N(c2ccc(-c3c([2H])c([2H])c4c([2H])c([2H])c5oc6c([2H])c([2H])c([2H])c([2H])c6c5c4c3[2H])cc2)c2ccc3c(c2)oc2ccccc23)c([2H])c1[2H]] 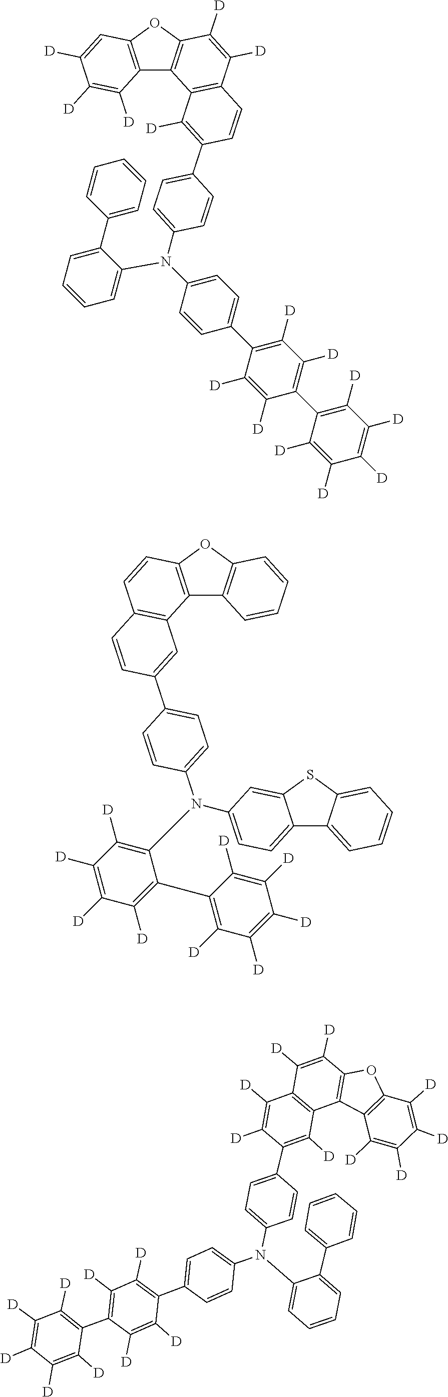 [SMILES: [2H]c1cc2oc3c([2H])c([2H])c4ccc(-c5ccc(N(c6ccc(-c7c([2H])c([2H])c(-c8c([2H])c([2H])c([2H])c([2H])c8[2H])c([2H])c7[2H])cc6)c6ccccc6-c6ccccc6)cc5)c([2H])c4c3c2c([2H])c1[2H] ; [2H]c1c([2H])c([2H])c(-c2c([2H])c([2H])c([2H])c([2H])c2N(c2ccc(-c3ccc4ccc5oc6ccccc6c5c4c3)cc2)c2ccc3c(c2)sc2ccccc23)c([2H])c1[2H] ; [2H]c1c([2H])c([2H])c(-c2c([2H])c([2H])c(-c3ccc(N(c4ccc(-c5c([2H])c([2H])c6c([2H])c([2H])c7oc8c([2H])c([2H])c([2H])c([2H])c8c7c6c5[2H])cc4)c4ccccc4-c4ccccc4)cc3)c([2H])c2[2H])c([2H])c1[2H]] 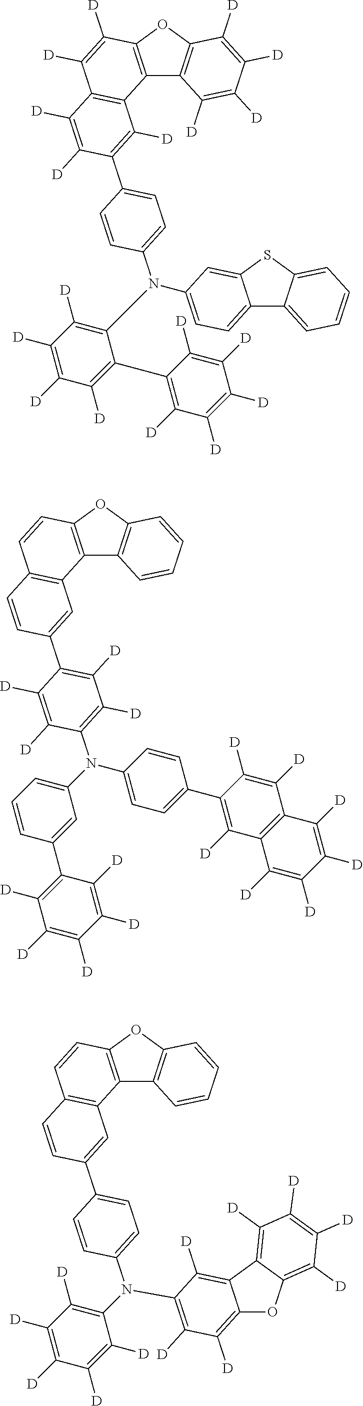 [SMILES: [2H]c1c([2H])c([2H])c(-c2c([2H])c([2H])c([2H])c([2H])c2N(c2ccc(-c3c([2H])c([2H])c4c([2H])c([2H])c5oc6c([2H])c([2H])c([2H])c([2H])c6c5c4c3[2H])cc2)c2ccc3c(c2)sc2ccccc23)c([2H])c1[2H] ; [2H]c1c([2H])c([2H])c(-c2cccc(N(c3ccc(-c4c([2H])c([2H])c5c([2H])c([2H])c([2H])c([2H])c5c4[2H])cc3)c3c([2H])c([2H])c(-c4ccc5ccc6oc7ccccc7c6c5c4)c([2H])c3[2H])c2)c([2H])c1[2H] ; [2H]c1c([2H])c([2H])c(N(c2ccc(-c3ccc4ccc5oc6ccccc6c5c4c3)cc2)c2c([2H])c([2H])c3oc4c([2H])c([2H])c([2H])c([2H])c4c3c2[2H])c([2H])c1[2H]] 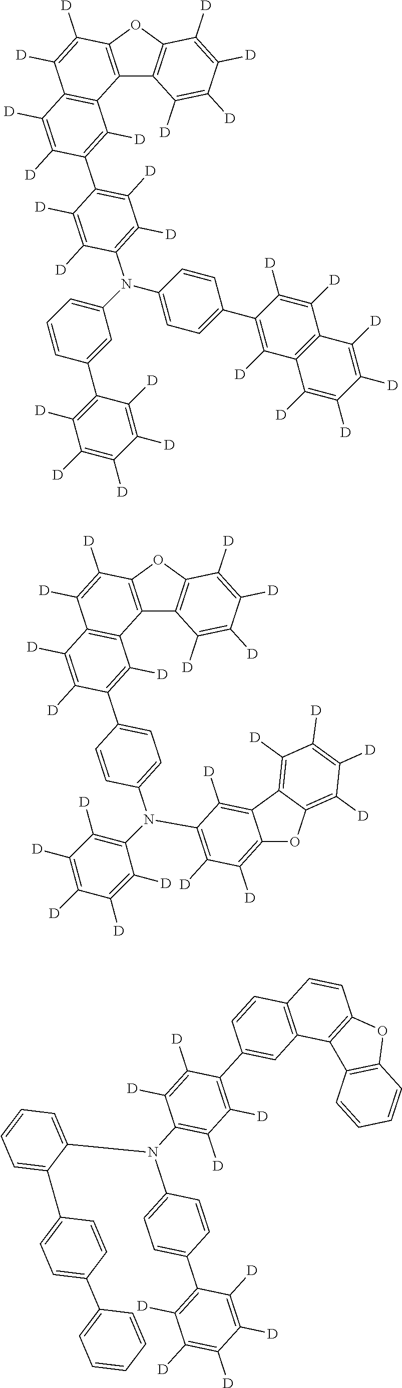 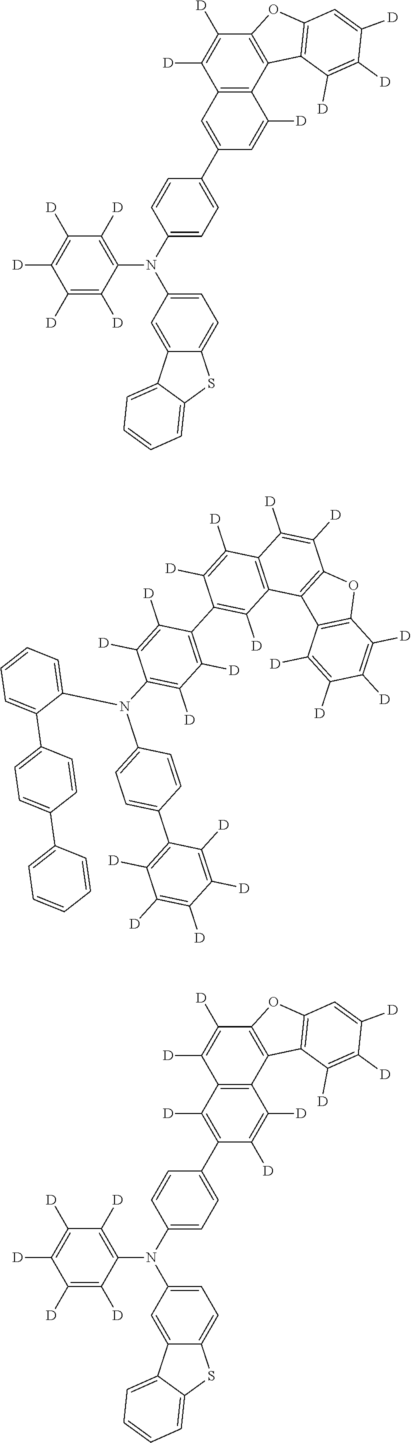 [SMILES: [2H]c1cc2oc3c([2H])c([2H])c4cc(-c5ccc(N(c6ccc7sc8ccccc8c7c6)c6c([2H])c([2H])c([2H])c([2H])c6[2H])cc5)cc([2H])c4c3c2c([2H])c1[2H] ; [2H]c1c([2H])c([2H])c(-c2ccc(N(c3ccccc3-c3ccc(-c4ccccc4)cc3)c3c([2H])c([2H])c(-c4c([2H])c([2H])c5c([2H])c([2H])c6oc7c([2H])c([2H])c([2H])c([2H])c7c6c5c4[2H])c([2H])c3[2H])cc2)c([2H])c1[2H] ; [2H]c1cc2oc3c([2H])c([2H])c4c([2H])c(-c5ccc(N(c6ccc7sc8ccccc8c7c6)c6c([2H])c([2H])c([2H])c([2H])c6[2H])cc5)c([2H])c([2H])c4c3c2c([2H])c1[2H]] 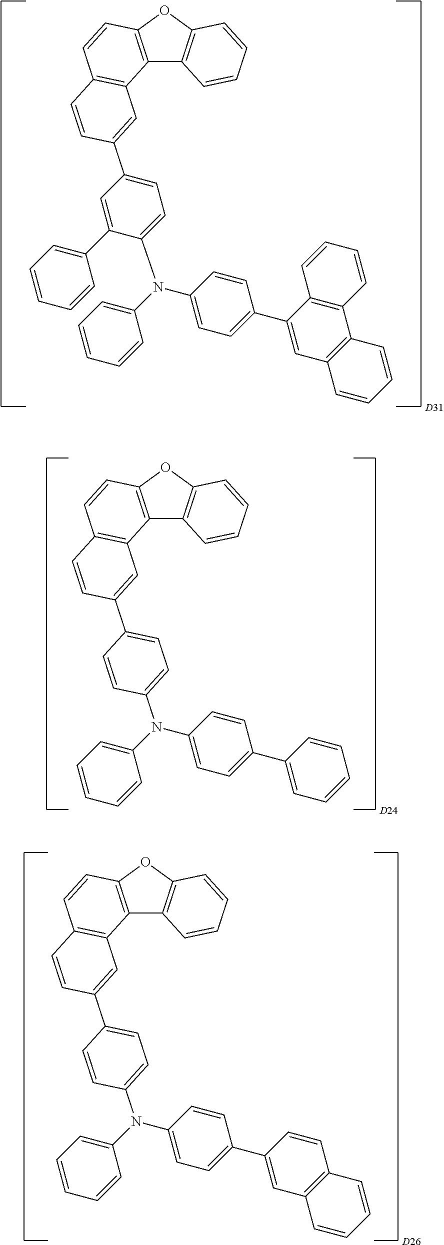 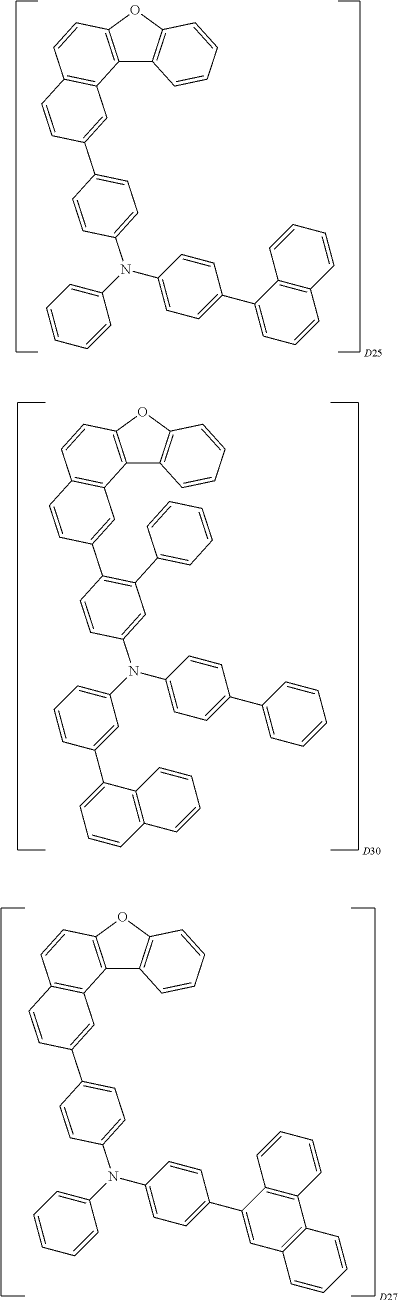 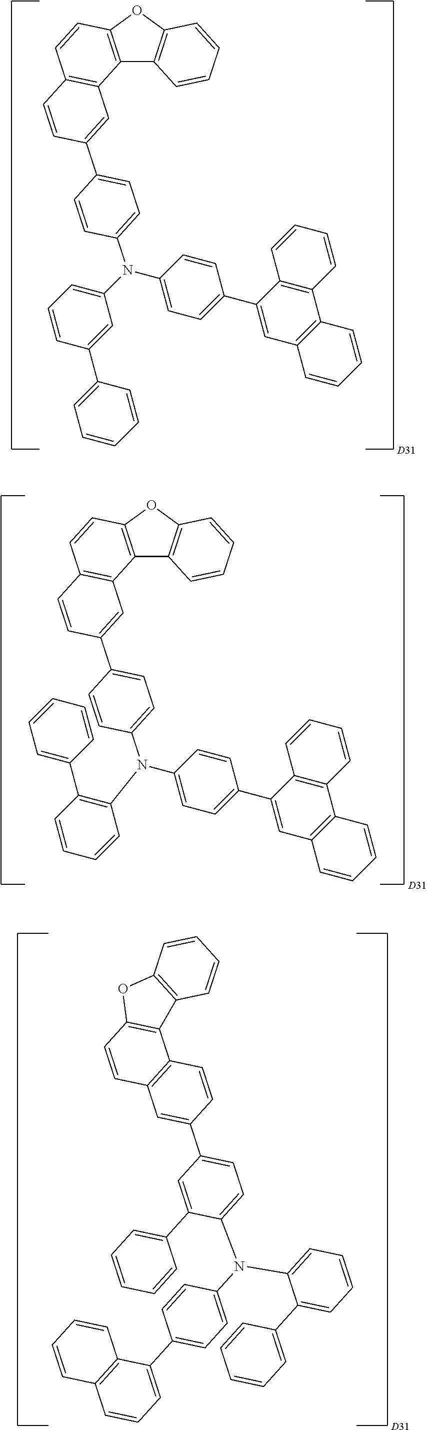 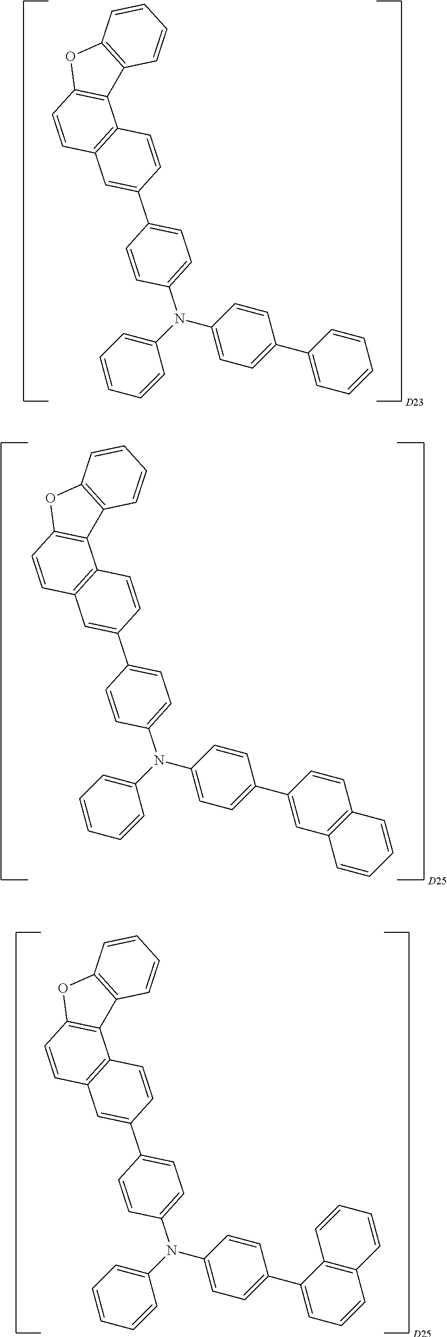 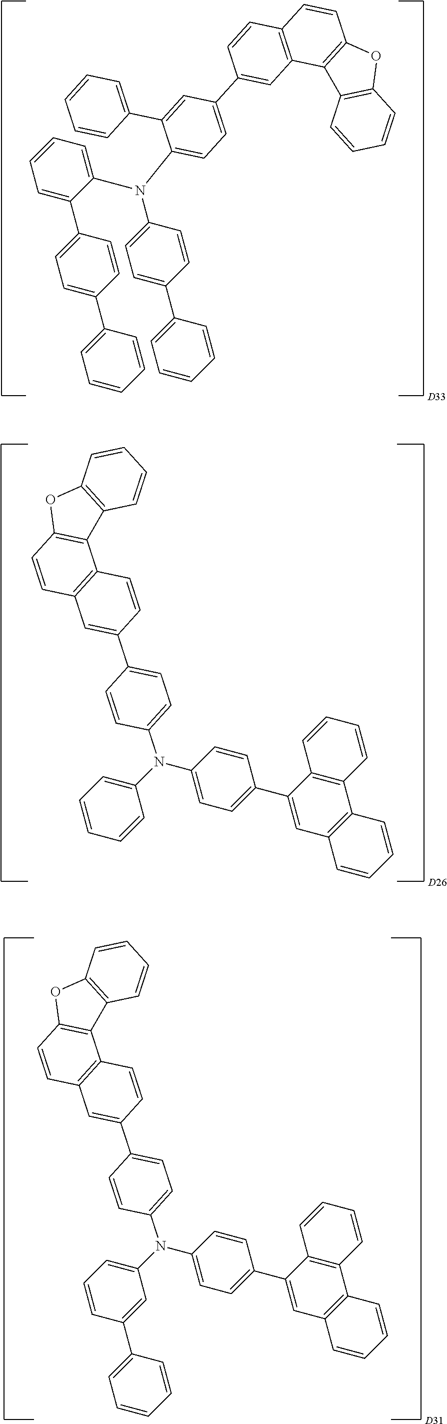 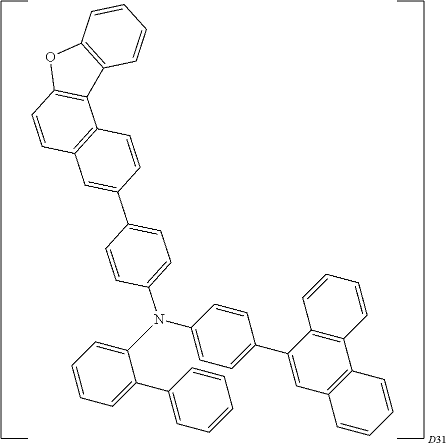 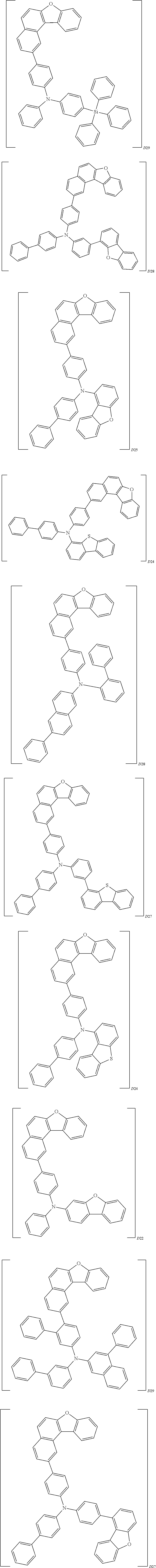 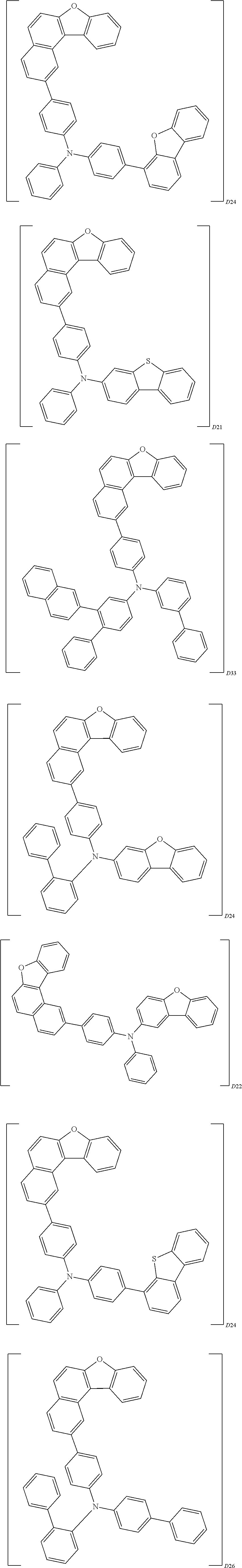 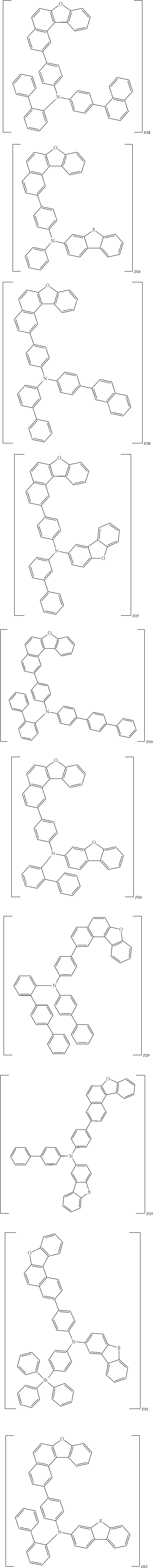 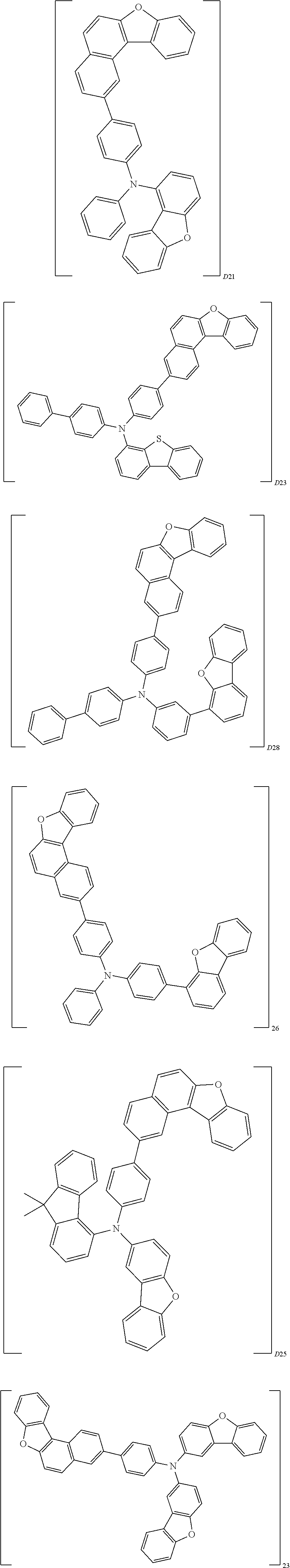 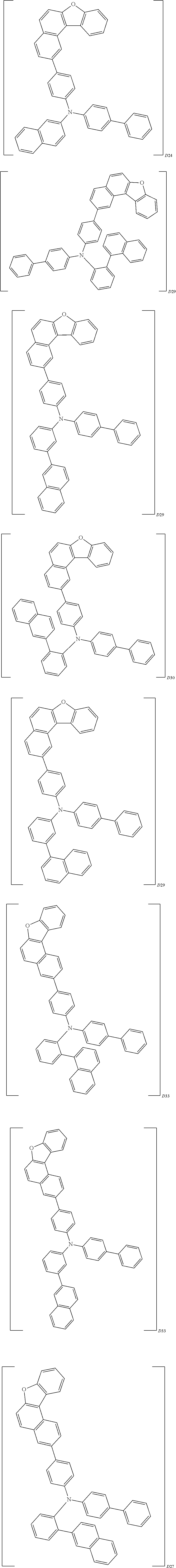 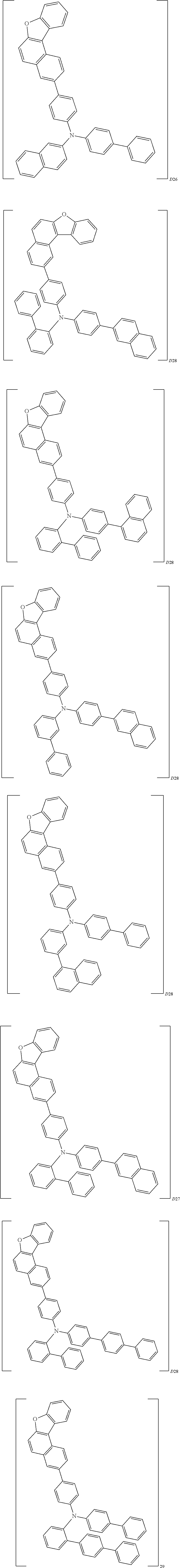 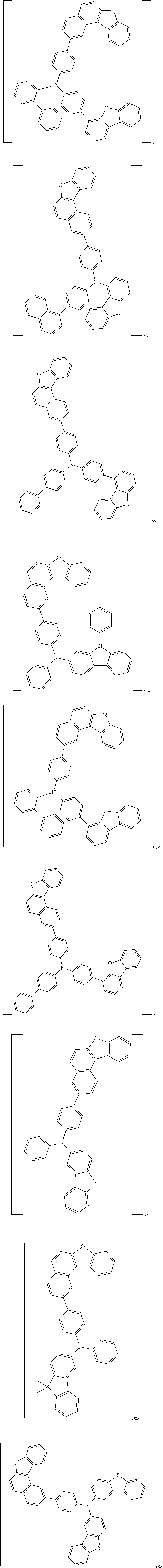 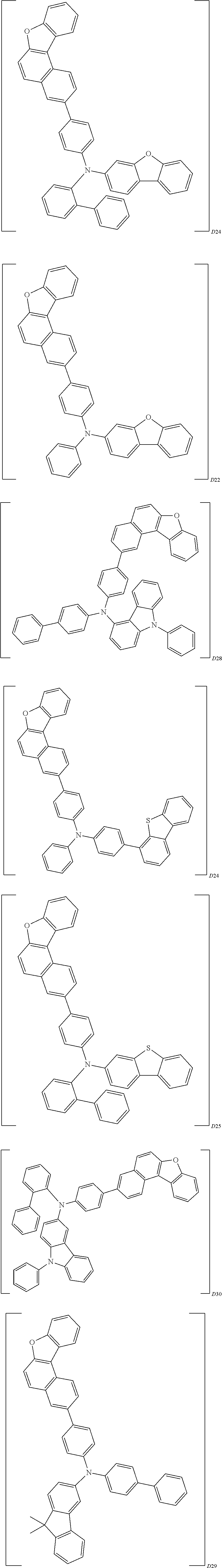 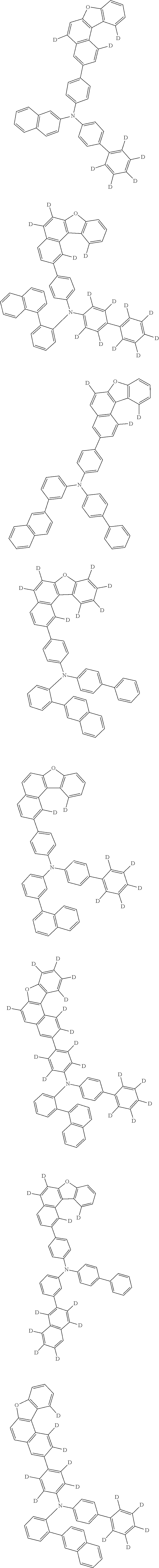 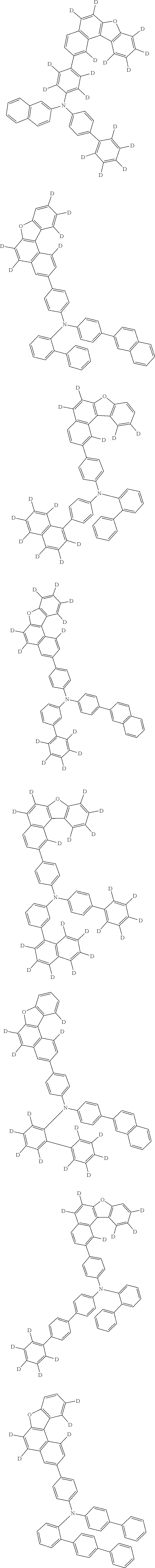 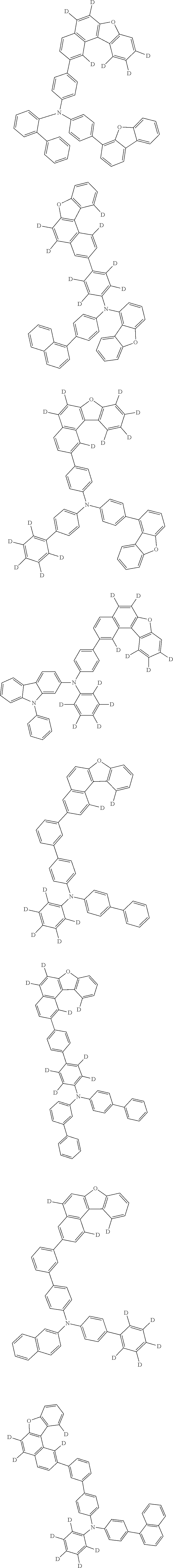 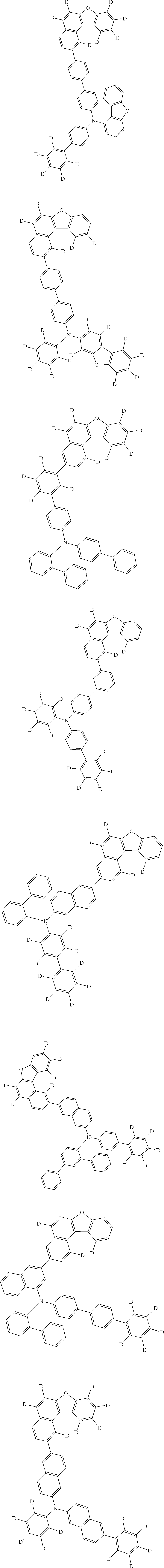 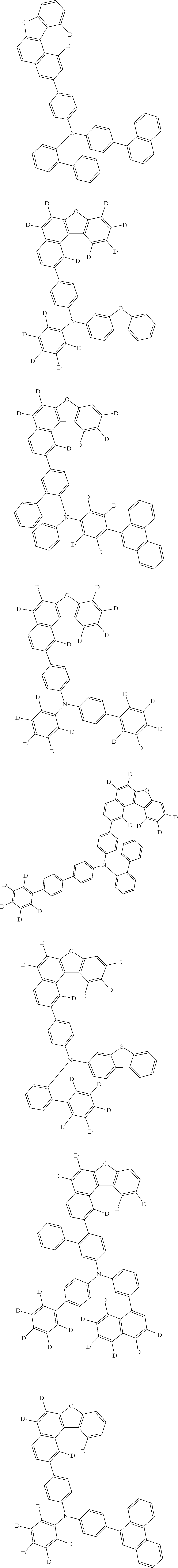 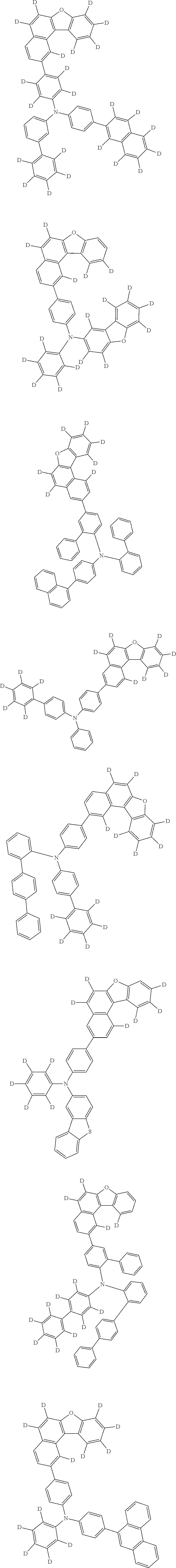 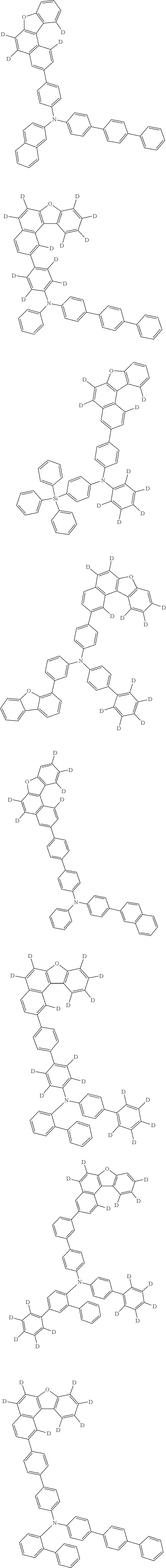 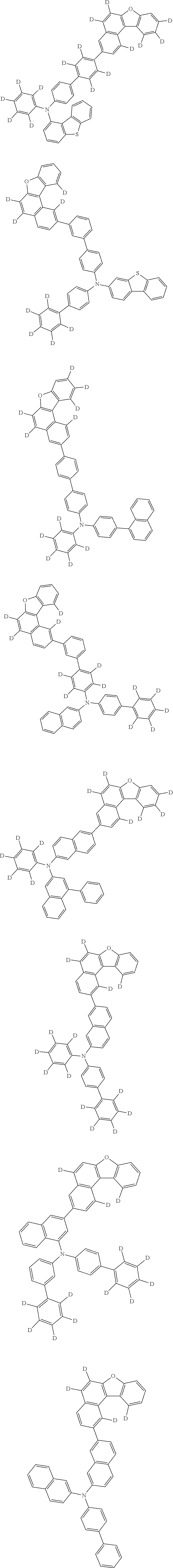 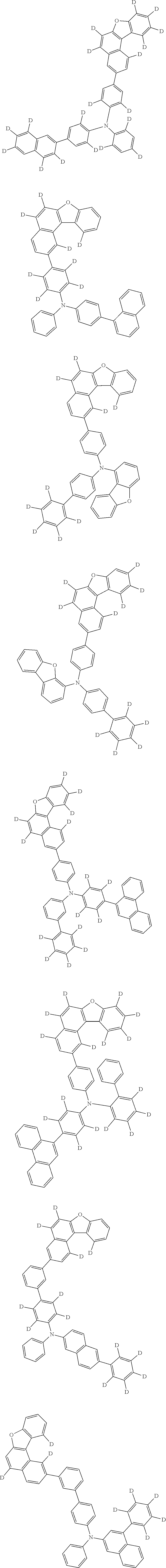 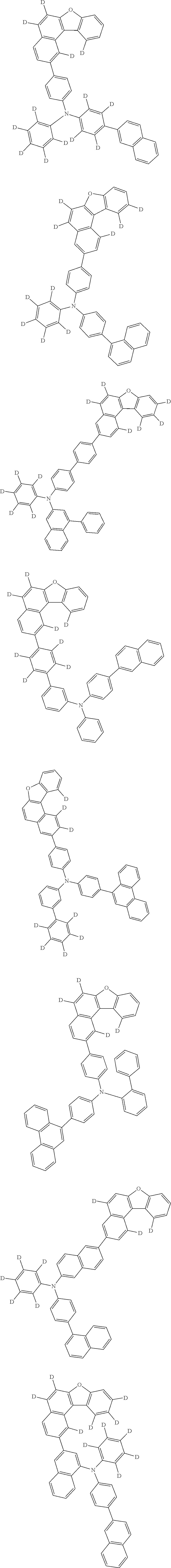 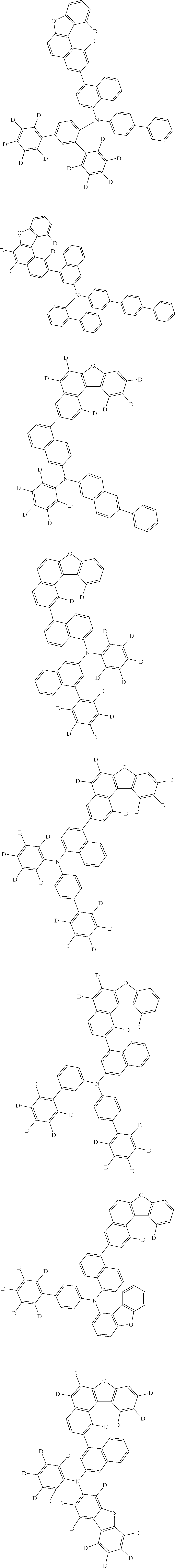 [chemical structure]
